(19) United States
(12) Patent Application Publication
(10) Pub. No.: US 2024/0059691 A1
(43) Pub. Date: Feb. 22, 2024

(54) ENZYME INHIBITORS

(71) Applicant: KALVISTA PHARMACEUTICALS LIMITED, Wiltshire (GB)

(72) Inventors: Mitchell Lewis CHILDS, Wiltshire (GB); Rebecca Louise DAVIE, Wiltshire (GB); Hannah Joy EDWARDS, Wiltshire (GB); David Michael EVANS, Wiltshire (GB); William Jack GREVES, Wiltshire (GB); Simon Teanby HODGSON, Ampthill (GB); Alessandro MAZZACANI, Hove (GB); Carl Leslie NORTH, Wiltshire (GB); Alicja Stela OBARA, Wiltshire (GB); Michael Bryan ROE, Wiltshire (GB); David Philip ROOKER, Wiltshire (GB); Michael John STOCKS, Leicestershire (GB); Louise Michelle BIRCH, Nottingham (GB); Mark PICHOWICZ, Nottingham (GB); Rachael PITTAWAY, Nottingham (GB); Alun John SMITH, Nottingham (GB); Adam Eric THROUP, Nottingham (GB); Joseph William WRIGGLESWORTH, Nottingham (GB); Xuezheng YANG, Nottingham (GB); David Edward CLARK, Harlow Essex (GB)

(21) Appl. No.: 18/254,952

(22) PCT Filed: Dec. 1, 2021
(86) PCT No.: PCT/GB2021/053137

§ 371 Date: May 30, 2023

Related U.S. Application Data
(60) Provisional application No. 63/120,074, filed on Dec. 1, 2020.

(30) Foreign Application Priority Data
Dec. 1, 2020 (GB) 2018970.0

(51) Int. Cl. C07D 487/04; C07D 471/04; C07D 401/12; C07D 401/14; C07D 487/08; C07D 471/08

(52) U.S. Cl. CPC C07D 487/04; C07D 471/04; C07D 401/12; C07D 401/14; C07D 487/08; C07D 471/08

(57) ABSTRACT

The present invention provides compounds of formula (I): compositions comprising such compounds; the use of such compounds in medicine; and methods of treating patients with such compounds; wherein A, W, R5, n, Z, X, Y and B are as defined herein. The present invention also relates to compounds useful as synthetic intermediates of compounds of formula (I).

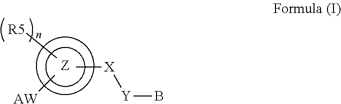

This invention relates to enzyme inhibitors that are inhibitors of Factor XIIa (FXIIa), and to pharmaceutical compositions comprising, and uses of, such inhibitors.

### BACKGROUND TO THE INVENTION

The compounds of the present invention are inhibitors of factor XIIa (FXIIa) and thus have a number of possible therapeutic applications, particularly in the treatment of diseases or conditions in which factor XIIa inhibition is implicated.

FXIIa is a serine protease (EC 3.4.21.38) derived from its zymogen precursor, factor XII (FXII), which is expressed by the F12 gene. Single chain FXII has a low level of amidolytic activity that is increased upon interaction with negatively charged surfaces and has been implicated in its activation (see Invanov et al., Blood. 2017 Mar. 16; 129(11):1527-1537. doi: 10.1182/blood-2016-10-744110). Proteolytic cleavage of FXII to heavy and light chains of FXIIa dramatically increases catalytic activity. FXIIa that retains its full heavy chain is αFXIIa. FXIIa that retains a small fragment of its heavy chain is βFXIIa. The separate catalytic activities of αFXIIa and βFXIIa contribute to the activation and biochemical functions of FXIIa. Mutations and polymorphisms in the F12 gene can alter the cleavage of FXII and FXIIa. FXIIa has a unique and specific structure that is different from many other serine proteases. For instance, the Tyr99 in FXIIa points towards the active site, partially blocking the S2 pocket and giving it a closed characteristic. Other serine proteases containing a Tyr99 residue (e.g. FXa, tPA and FIXa) have a more open S2 pocket. Moreover, in several trypsin-like serine proteases the P4 pocket is lined by an “aromatic box” which is responsible for the P4-driven activity and selectivity of the corresponding inhibitors. However, FXIIa has an incomplete “aromatic box” resulting in more open P4 pocket. See e.g. “Crystal structures of the recombinant β-factor XIIa protease with bound Thr-Arg and Pro-Arg substrate mimetics” M. Pathak et al., Acta. Cryst.2019, D75, 1-14; “Structures of human plasma β-factor XIIa cocrystallized with potent inhibitors” A Dementiev et al., Blood Advances 2018, 2(5), 549-558; “Design of Small-Molecule Active-Site Inhibitors of the S1A Family Proteases as Procoagulant and Anticoagulant Drugs” P. M. Fischer, J. Med. Chem., 2018, 61(9), 3799-3822; “Assessment of the protein interaction between coagulation factor XII and corn trypsin inhibitor by molecular docking and biochemical validation” B. K. Hamad et al. Journal of Thrombosis and Haemostasis, 15: 1818-1828.

FXIIa converts plasma prekallikrein (PK) to plasma kallikrein (PKa), which provides positive feedback activation of FXII to FXIIa. FXII, PK, and high molecular weight kininogen (HK) together represent the contact system. FXIIa mediated conversion of plasma prekallikrein to plasma kallikrein can cause subsequent cleavage of HK to generate bradykinin, a potent inflammatory hormone that can also increase vascular permeability, which has been implicated in disorders such as hereditary angioedema (HAE). The contact system is activated via a number of mechanisms, including interactions with negatively charged surfaces, negatively charged molecules, unfolded proteins, artificial surfaces, foreign tissue (e.g. biological transplants, that include bio-prosthetic heart valves, and organ/tissue transplants), bacteria, and biological surfaces (including endothelium and extracellular matrix) that mediate assembly of contact system components. In addition, the contact system is activated by plasmin, and cleavage of FXII by other enzymes can facilitate its activation.

Activation of the contact system leads to activation of the kallikrein kinin system (KKS), complement system, and intrinsic coagulation pathway (see https://www.genome.jp/kegg-bin/show pathway?map04610). In addition, FXIIa has additional substrates both directly, and indirectly via PKa, including Proteinase-activated receptors (PARs), plasminogen, and neuropeptide Y (NPY) which can contribute to the biological activity of FXIIa. Inhibition of FXIIa could provide clinical benefits by treating diseases and conditions associated with these systems, pathways, receptors, and hormones.

PKa activation of PAR2 mediates neuroinflammation and may contribute to neuroinflammatory disorders including multiple sclerosis (see Göbel et al., Proc Natl Acad Sci USA. 2019 Jan. 2; 116(1):271-276. doi: 10.1073/pnas.1810020116). PKa activation of PAR1 and PAR2 on vascular smooth muscle cells has been implicated in vascular hypertrophy and atherosclerosis (see Abdallah et al., J Biol Chem. 2010 Nov. 5; 285(45):35206-15. doi: 10.1074/jbc.M110.171769). FXIIa activation of plasminogen to plasmin contributes to fibrinolysis (see Konings et al., Thromb Res. 2015 August; 136(2):474-80. doi: 10.1016/j.thromres.2015.06.028). PKa proteolytically cleaves NPY and thereby alters its binding to NPY receptors (Abid et al., J Biol Chem. 2009 Sep. 11; 284(37):24715-24. doi: 10.1074/jbc.M109.035253). Inhibition of FXIIa could provide clinical benefits by treating diseases and conditions caused by PAR signaling, NPY metabolism, and plasminogen activation.

FXIIa-mediated activation of the KKS results in the production of bradykinin (BK), which can mediate, for example, angioedema, pain, inflammation, vascular hyperpermeability, and vasodilatation (see Kaplan et al., Adv Immunol. 2014; 121:41-89. doi: 10.1016/B978-0-12-800100-4.00002-7; and Hopp et al., J Neuroinflammation. 2017 Feb. 20; 14(1):39. doi: 10.1186/s12974-017-0815-8). Garadacimab (CSL-312), a monoclonal antibody inhibitory against FXIIa, recently completed a phase 2 study where monthly prophylactic subcutaneous treatment was reported to be well tolerated and effective in preventing attacks in patients with type I/II hereditary angioedema (HAE), which results in intermittent swelling of face, hands, throat, gastro-intestinal tract and genitals (see https://www.clinicaltrials.gov/ct2/show/NCT03712228 and Craig et al., 1451, Allergy. 2020; 75(Suppl. 109):5-99. doi: 10.1111/a11.14504). Mutations in FXII that facilitate its activation to FXIIa have been identified as a cause of HAE (see Björkqvist et al., J Clin Invest. 2015 Aug. 3; 125(8):3132-46. doi: 10.1172/JC177139; and de Maat et al., J Allergy Clin Immunol. 2016 November; 138(5):1414-1423.e9. doi: 10.1016/j.jaci.2016.02.021). Since FXIIa mediates the generation of PK to PKa, inhibitors of FXIIa could provide protective effects of all form of BK-mediated angioedema, including HAE and non-hereditary bradykinin-mediated angioedema (BK-AEnH).

“Hereditary angioedema” can be defined as any disorder characterised by recurrent episodes of bradykinin-mediated angioedema (e.g. severe swelling) caused by an inherited genetic dysfunction/fault/mutation. There are currently three known categories of HAE: (i) HAE type 1, (ii) HAE type 2, and (iii) normal C1 inhibitor HAE (normal C1-Inh HAE). However, work on characterizing the etiologies of HAE is ongoing so it is expected that further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor. For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al. 2014 The Association for the Publication of the Journal of Internal Medicine Journal of Internal Medicine, 2015, 277; 585-593; or Maat et al. J Thromb Haemost. 2019 January; 17(1):183-194. doi: 10.1111/jth.14325).

However, angioedemas are not necessarily inherited. Indeed, another class of angioedema is bradykinin mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which, without being bound by theory, is thought to be on account of the shared bradykinin mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non hereditary angioedema with normal C1 Inhibitor (AE-nC1 Inh), which can be environmental, hormonal, or drug induced; acquired angioedema; anaphylaxis associated angioedema; angiotensin converting enzyme (ACE) inhibitor induced angioedema; dipeptidyl peptidase 4 inhibitor induced angioedema; and tPA induced angioedema (tissue plasminogen activator induced angioedema). However, reasons why these factors and conditions cause angioedema in only a relatively small proportion of individuals are unknown.

Environmental factors that can induce AE-nC1 Inh include air pollution (Kedarisetty et al, Otolaryngol Head Neck Surg. 2019 April 30:194599819846446. doi: 10.1177/0194599819846446) and silver nanoparticles such as those used as antibacterial components in healthcare, biomedical and consumer products (Long et al., Nanotoxicology. 2016; 10(4):501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015; Leibfried and Kovary. J Pharm Pract 2017); van den Elzen et al. (Clinic Rev Allerg Immunol 2018); Han et al (JCI 2002).

For instance, BK-medicated AE can be caused by thrombolytic therapy. For example, tPA induced angioedema is discussed in various publications as being a potentially life threatening complication following thrombolytic therapy in acute stroke victims (see e.g. Simso et al., Blood. 2017 Apr. 20; 129(16):2280-2290. doi: 10.1182/blood-2016-09-740670; Fröhlich et al., Stroke. 2019 Jun. 11:STROKEAHA119025260. doi: 10.1161/STROKEAHA.119.025260; Rathbun, Oxf Med Case Reports. 2019 Jan. 24; 2019(1):omy112. doi: 10.1093/omcr/omy112; Lekoubou et al., Neurol Res. 2014 July; 36(7):687-94. doi: 10.1179/1743132813Y.0000000302; Hill et al., Neurology. 2003 May 13; 60(9):1525-7).

Stone et al. (Immunol Allergy Clin North Am. 2017 August; 37(3):483-495.) reports that certain drugs can cause angioedema.

Scott et al. (Curr Diabetes Rev. 2018; 14(4):327-333. doi: 10.2174/1573399813666170214113856) reports cases of dipeptidyl Peptidase-4 Inhibitor induced angioedema.

Hermanrud et al., (BMJ Case Rep. 2017 Jan. 10; 2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 January; 124(1):115-122. doi: 10.1111/bcpt.13097) reports angiotensin II receptor blocker (ARB)-related angioedema. Reichman et al., (Pharmacoepidemiol Drug Saf. 2017 October; 26(10):1190-1196. doi: 10.1002/pds.4260) also reports angioedema risk for patients taking ACE inhibitors, ARB inhibitors and beta blockers. Diestro et al. (J Stroke Cerebrovasc Dis. 2019 May; 28(5):e44-e45. doi: 10.1016/j.jstrokecerebrovasdis.2019.01.030) also reports a possible association between certain angioedemas and ARBs.

Giard et al. (Dermatology. 2012; 225(1):62-9. doi: 10.1159/000340029) reports that bradykinin mediated angioedema can be precipitated by estrogen contraception, so called “oestrogen associated angioedema”.

Contact system mediated activation of the KKS has also been implicated in retinal edema and diabetic retinopathy (see Liu et al., Biol Chem. 2013 March; 394(3):319-28. doi: 10.1515/hsz-2012-0316). FXIIa concentrations are increased in the vitreous fluid from patients with advance diabetic retinopathy and in Diabetic Macular Edema (DME) (see Gao et al., Nat Med. 2007 February; 13(2):181-8. Epub 2007 January 28 and Gao et al., J Proteome Res. 2008 June; 7(6):2516-25. doi: 10.1021/pr800112 g). FXIIa has been implicated in mediating both vascular endothelial growth factor (VEGF) independent DME (see Kita et al., Diabetes. 2015 October; 64(10):3588-99. doi: 10.2337/db15-0317) and VEGF mediated DME (see Clermont et al., Invest Ophthalmol Vis Sci. 2016 May 1; 57(6):2390-9. doi: 10.1167/iovs.15-18272). FXII deficiency is protective against VEGF induced retinal edema in mice (Clermont et al., ARVO talk 2019). Therefore it has been proposed that FXIIa inhibition will provide therapeutic effects for diabetic retinopathy and retinal edema caused by retinal vascular hyperpermeability, including DME, retinal vein occlusion, age-related macular degeneration (AMD).

As noted above, the contact system can be activated by interaction with bacteria, and therefore FXIIa has been implicated in the treatment of sepsis and bacterial sepsis (see Morrison et al., J Exp Med. 1974 Sep. 1; 140(3):797-811). Therefore, FXIIa inhibitors could provide therapeutic benefits in treating sepsis, bacterial sepsis and disseminated intravascular coagulation (DIC).

FXIIa mediated activation of the KKS and production of BK have been implicated in neurodegenerative diseases including Alzheimer's disease, multiple sclerosis, epilepsy and migraine (see Zamolodchikov et al., Proc Natl Acad Sci USA. 2015 Mar. 31; 112(13):4068-73. doi: 10.1073/pnas.1423764112; Simões et al., J Neurochem. 2019 August; 150(3):296-311. doi: 10.1111/jnc.14793; Göbel et al., Nat Commun. 2016 May 18; 7:11626. doi: 10.1038/ncomms11626; and https://clinicaltrials.gov/ct2/show/NCT03108469). Therefore, FXIIa inhibitors could provide therapeutic benefits in reducing the progression and clinical symptoms of these neurodegenerative diseases.

FXIIa has also been implicated in anaphylaxis (see Bender et al., Front Immunol. 2017 Sep. 15; 8:1115. doi: 10.3389/fimmu.2017.01115; and Sala-Cunill et al., J Allergy Clin Immunol. 2015 April; 135(4):1031-43.e6. doi: 10.1016/j.jaci.2014.07.057). Therefore, FXIIa inhibitors could provide therapeutic benefits in reducing the clinical severity and incidence of anaphylactic reactions.

The role of FXIIa in coagulation was identified over 50 years ago, and has been extensively documented in publications using biochemical, pharmacological, genetic and molecular studies (see Davie et al., Science. 1964 Sep. 18; 145(3638):1310-2). FXIIa mediated activation of factor XI (FXI) triggers the intrinsic coagulation pathway. In addition, FXIIa can increase coagulation in a FXI independent manner (see Radcliffe et al., Blood. 1977 October; 50(4):611-7; and Puy et al., J Thromb Haemost. 2013 July; 11(7):1341-52. doi: 10.1111/jth.12295). Studies on both humans and experimental animal models have demonstrated that FXII deficiency prolongs activated partial prothrombin time (APTT) without adversely affecting hemostasis (see Renne et al., J Exp Med. 2005 Jul. 18; 202(2):271-81; and Simão et al., Front Med (Lausanne). 2017 Jul. 31; 4:121. doi: 10.3389/fmed.2017.00121). Pharmacological inhibition of FXIIa also prolongs APTT without increasing bleeding (see Worm et al., Ann Transl Med. 2015 October; 3(17):247. doi: 10.3978/j.issn.2305-5839.2015.09.07). These data suggest that inhibition of FXIIa could provide therapeutic effects against thrombosis without inhibiting bleeding. Therefore, FXIIa inhibitors could be used to treat a spectrum of prothrombotic conditions including venous thromboembolism (VTE); cancer associated thrombosis; complications caused by mechanical and bioprosthetic heart valves, catheters, extracorporeal membrane oxygenation (ECMO), left ventricular assisted devices (LVAD), dialysis, cardiopulmonary bypass (CPB); sickle cell disease, joint arthroplasty, thrombosis induced by tPA, Paget-Schroetter syndrome and Budd-Chari syndrome. FXIIa inhibitor could be used for the treatment and/or prevention of thrombosis, edema, and inflammation associated with these conditions.

Surfaces of medical devices that come into contact with blood can cause thrombosis. FXIIa inhibitors may also be useful for treating or preventing thromboembolism by lowering the propensity of devices that come into contact with blood to clot blood. Examples of devices that come into contact with blood include vascular grafts, stents, in-dwelling catheters, external catheters, orthopedic prosthesis, cardiac prosthesis, and extracorporeal circulation systems.

Preclinical studies have shown that FXIIa has been shown to contribute to stroke and its complications following both ischemic stroke, and hemorrhagic accidents (see Barbieri et al., J Pharmacol Exp Ther. 2017 March; 360(3):466-475. doi: 10.1124/jpet.116.238493; Krupka et al., PLoS One. 2016 Jan. 27; 11(1):e0146783. doi: 10.1371/journal.pone.0146783; Leung et al., Transl Stroke Res. 2012 September; 3(3):381-9. doi: 10.1007/s12975-012-0186-5; Simso et al., Blood. 2017 Apr. 20; 129(16):2280-2290. doi: 10.1182/blood-2016-09-740670; and Liu et al., Nat Med. 2011 February; 17(2):206-10. doi: 10.1038/nm.2295). Therefore, FXIIa inhibition may improve clinical neurological outcomes in the treatment of patients with stroke.

FXII deficiency has been shown to reduce the formation of atherosclerotic lesions in Apoe^{-/-} mice (Didiasova et al., Cell Signal. 2018 November; 51:257-265. doi: 10.1016/j.cellsig.2018.08.006). Therefore, FXIIa inhibitors could be used in the treatment of atherosclerosis.

FXIIa, either directly, or indirectly via PKa, has been shown to activate the complement system (Ghebrehiwet et al., Immunol Rev. 2016 November; 274(1):281-289. doi: 10.1111/imr.12469). BK increases complement C3 in the retina, and an in vitreous increase in complement C3 is associated with DME (Murugesan et al., Exp Eye Res. 2019 Jul. 24; 186:107744. doi: 10.1016/j.exer.2019.107744). Both FXIIa and PKa activate the complement system (see Irmscher et al., J Innate Immun. 2018; 10(2):94-105. doi: 10.1159/000484257; and Ghebrehiwet et al., J Exp Med. 1981 Mar. 1; 153(3):665-76).

A phase 2 study to assess the safety and efficacy of CSL312, a FXIIa inhibitor, in the treatment of COVID-19 has been assigned clinicaltrials.gov identifier NCT04409509. Shatzel et al. (Res Pract Thromb Haemost, 2020 May 15; 4(4):500-505. doi: 10.1002/rth2.12349) also relates to investigating the contact system's role in COVID-19.

Wygrecka et al. (“Coagulation factor XII regulates inflammatory responses in human lungs”, European Respiratory Journal 2017 50: PA339; DOI: 10.1183/1393003.congress-2017.PA339) relates to the effect of an accumulation of FXII in acute respiratory distress syndrome (ARDS) lungs.

Wong et al. (“CSL312, a Novel Anti-FXII Antibody, Blocks FXII-Induced IL-6 Production from Primary Non-Diseased and Idiopathic Pulmonary Fibrosis Fibroblasts”, American Journal of Respiratory and Critical Care Medicine 2020; 201:A6363) reports that activated FXII may contribute to lung fibrosis (e.g. idiopathic Pulmonary Fibrosis) through direct stimulation of fibroblasts to produce pro-fibrotic cytokine IL-6.

Göbel et al. (The Coagulation Factors Fibrinogen, Thrombin, and Factor XII in Inflammatory Disorders-A Systematic Review, Front. Immunol., 26 Jul. 2018|https://doi.org/10.3389/fimmu.2018.01731) relates to FXII's role in the rheumatoid arthritis (RA).

Scheffel et al. (Cold-induced urticarial autoinflammatory syndrome related to factor XII activation, Nature Communications volume 11, Article number: 179 (2020)) reports that there is a link between contact system activation and cytokine-mediated inflammation, such as cold-induced urticarial autoinflammatory syndrome.

Compounds that are said to be FXIIa inhibitors have been described by Rao et al. (“Factor XIIa Inhibitors” WO2018/093695), Hicks et al. (“Factor XIIa Inhibitors” WO2018/093716), Breslow et al. (“Aminotriazole immunomodulators for treating autoimmune diseases” WO2017/123518) and Ponda et al. (“Aminacylindazole immunomodulators for treatment of autoimmune diseases” WO2017/205296 and “Pyranopyrazole and pyrazolopyridine immunomodulators for treatment of autoimmune diseases” WO2019/108565). FXII/FXIIa inhibitors are said to have been described by Nolte et al. (“Factor XII inhibitors for the administration with medical procedures comprising contact with artificial surfaces” WO2012/120128).

Compounds that are said to be modulators of FXIIa have been described by Philippou et al. (“Factor XIIa Inhibitors” WO 2019/211585 and WO 2019/186164). Macrocylic peptides that are said to be inhibitors of FXIIa have been described by Wilbs et al. (Nat Commun 11, 3890 (2020). Doi: 10.1038/s41467-020-17648-w).

To date, no FXIIa inhibitors have been approved for medical use, and there are no small molecule FXIIa inhibitors in clinical development. Although certain known compounds are said to be modulators or inhibitors of FXIIa, these compounds can suffer from limitations such as being non-reversible or covalent binders, being poorly selective for FXIIa over other related enzymes, or not having demonstrated pharmacokinetic properties suitable for oral therapy. For example, compounds with acylating reactivity e.g. acylated aminotriazoles, are typically non-reversible covalent binders, and can sometimes also be unstable in water and/or blood plasma due to their inherent reactivity. Poor selectivity for FXIIa over other serine proteases (such as thrombin, FXa, FXIa, KLK1, plasmin, trypsin) increases the risk of off-target effects, which can be made even worse (i.e. there is typically a higher likelihood of poor selectivity and off-target effects) if the inhibitor is a covalent binder. Therefore, there remains a need to develop new FXIIa inhibitors that are not covalent inhibitors and/or are highly selective for FXIIa in order to e.g. mitigate the risks of non-selectivity and cytotoxicity. There is a particular need to develop small molecule FXIIa inhibitors as an oral therapy.

In view of the above, there also remains a need to develop new FXIIa inhibitors that will have utility to treat a wide range of disorders, in particular angioedema; HAE, including: (i) HAE type 1, (ii) HAE type 2, and (iii) normal C1 inhibitor HAE (normal C1-Inh HAE); BK-AEnH, including AE-nC11 nh, ACE and tPA induced angioedema; vascular hyperpermeability; stroke including ischemic stroke and haemorrhagic accidents; retinal edema; diabetic retinopathy; DME; retinal vein occlusion; AMD; neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; anaphylaxis; thrombosis; thromboembolism caused by increased propensity of medical devices that come into contact with blood to clot blood; prothrombotic conditions including disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; acute respiratory distress syndrome (ARDS); idiopathic pulmonary fibrosis (IPF); rheumatoid arthritis (RA); and cold-induced urticarial autoinflammatory syndrome. In particular, there remains a need to develop new FXIIa inhibitors.

### DESCRIPTION OF THE INVENTION

The present invention relates to a series of inhibitors of Factor XIIa (FXIIa). The compounds of the invention are potentially useful in the treatment of diseases or conditions in which factor XIIa inhibition is implicated. The invention further relates to pharmaceutical compositions of the inhibitors, to the use of the compositions as therapeutic agents, and to methods of treatment using these compositions. The invention also relates to compounds useful as intermediates in the synthesis of the inhibitors of FXIIa of the invention described herein.

A first aspect of the invention provides compounds of formula (I)

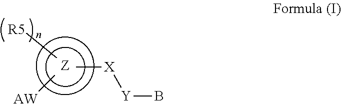

wherein:
Z is a 6- or 5-membered heteroaromatic ring containing 1, 2 or 3 ring members independently selected from N, S and O; or phenyl; or,
Z is 2-pyridone or 4-pyridone,
X is selected from SO₂ and CR1R2;
R1 is selected from H, alkyl, alkoxy, OH, halo and NR13R14; and
R2 is selected from H and small alkyl; or
R1 and R2, together with the carbon atom to which they are attached, are linked by alkylene to form a 3-, 4-, or 5-membered saturated ring;
Y is selected from NR12, 0, and CR3R4;
R3 and R4 are independently selected from H and alkyl; or
X is CR1R2 and Y is CR3R4, and R1 and R3, together with the carbon atom to which R1 is attached and the carbon atom to which R3 is attached, are linked by alkylene to form a 3-, 4-, or 5-membered saturated ring; or
X is CR1R2 and Y is NR12, and R1 and R12, together with the carbon atom to which R1 is attached and the nitrogen atom to which R12 is attached, are linked by alkylene to form a 3-, 4-, or 5-membered saturated heterocycle;
B is selected from:
   (i) heteroaryl^{a};
   (ii) aryl;
   (iii) a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃; and
   (iv) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring;
n is 0, 1 or 2;
when present, each R5 is independently selected from alkyl, cyclopropyl, alkoxy, halo, OH, CN, (CH₂)₀₋₆COOH, and CF₃;
AW- is selected from:
—(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—NR12-(CH₂)₁₋₆—C(═O)-A, —(CH₂)₀₋₆—NH—C(═O)—(CH₂)₀₋₆-A, —C(═O)NR12-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A and —SO₂—NH-A;
A is a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro; wherein when A is a tricyclic ring system, each of the three rings in the tricyclic ring system is either fused, bridged or spiro to at least one of the other rings in the tricyclic ring system; alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from (C₁-C₆)alkoxy, OH, —NR13R14, —C(═O)OR13, —C(═O)NR13R14, CN, CF₃, halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from (C₁-C₆)alkoxy, OH, CN, CF₃, halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from (C1-C6)alkoxy, OH, NR13R14, C(═O)OR13, C(═O)NR13R14, CN, CF3, halo;
small alkyl^{b} is linear saturated hydrocarbon having up to 4 carbon atoms (C₁-C₄) or a branched saturated hydrocarbon of between 3 and 4 carbon atoms (C₃-C₄); small alkyl^{b} may optionally be substituted with 1 or 2 substituents independently selected from (C₁-C₆)alkoxy, OH, CN, CF₃, halo;
alkylene is a bivalent linear saturated hydrocarbon having 1 to 5 carbon atoms (C₁-C₅); alkylene may optionally be substituted with 1 or 2 substituents independently selected from alkyl^{b}, (C₁-C₆)alkoxy, OH, CN, CF₃, halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ and CF₃;
aryl^{b} is phenyl, biphenyl or naphthyl; aryl^{b} may be optionally substituted with 1, 2 or 3 substituents independently selected from alkyl^{b}, alkoxy, OH, halo, CN, and CF₃; cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, halo;
alkoxy is a linear O-linked hydrocarbon of between 1 and 6 carbon atoms (C1-C₆) or a branched O-linked hydrocarbon of between 3 and 6 carbon atoms (C₃-C₆); alkoxy may optionally be substituted with 1 or 2 substituents independently selected from OH, CN, CF₃, and fluoro;
halo is F, Cl, Br, or I;
heteroaryl is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃;
heteroaryl^{a} is a 5, 6, 9 or 10 membered mono- or bi-cyclic aromatic ring, containing, where possible, 1, 2, 3 or 4 ring members independently selected from N, NR12, S and O; heteroaryl^{a} may be optionally substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃;
heteroaryl^{b} is a 5, 6, 9 or 10 membered mono- or bi-cyclic aromatic ring, containing, where possible, 1, 2 or 3 ring members independently selected from N, NR12, S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃;
heterocycloalkyl is a non-aromatic carbon-containing monocyclic ring containing 5, 6, or 7 ring members, wherein one or two ring members are independently selected from N, NR8, S, SO, SO₂, and O; wherein heterocycloalkyl may be optionally substituted with 1, 2, or 3 substituents independently selected from alkyl, alkoxy, OH, OCF₃, halo, oxo and CN;
R8 is independently selected from H, alkyl, cycloalkyl, or heterocycloalkyl^{a};
heterocycloalkyl^{a} is a non-aromatic carbon-containing monocyclic ring containing 3, 4, 5, or 6, ring members, wherein at least one ring member is independently selected from N, NR12, S, and O; heterocycloalkyl^{a} may be optionally be substituted with 1 or 2 substituents independently selected from alkyl, (C₁-C₆)alkoxy, OH, CN, CF₃, halo;
R12 is independently selected from H, alkyl, or cycloalkyl;
R13 and R14 are independently selected from H, alkyl^{b}, aryl^{b} and heteroaryl^{b} or R13 and R14 together with the nitrogen atom to which they are attached form a carbon-containing 4-, 5-, 6- or 7-membered heterocyclic ring, optionally containing an additional heteroatom selected from N, NR12, S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
and tautomers, isomers, stereoisomers (including enantiomers, diastereoisomers and racemic and scalemic mixtures thereof), deuterated isotopes, and pharmaceutically acceptable salts and/or solvates thereof.

The compounds of the formula (I) have been developed to be inhibitors of FXIIa, which as noted above, has a unique and specific binding site and there is a need for small molecule FXIIa inhibitors. Furthermore, the compounds of formula (I) have been carefully developed to (i) show selectivity for FXIIa over other serine proteases, thus reducing the risk of off-target effects and cytotoxicity, and (ii) to possess characteristics that can be considered suitable for oral delivery e.g. a suitable oral availability profile. The compounds of formula (I) can also avoid including groups associated with covalent binding properties e.g. groups with acylating reactivity such as acylated aminotriazoles, and thus can provide compounds that are reversible inhibitors, to further reduce the risk of off-target effects and cytotoxicity.

The present invention also provides a prodrug of a compound as herein defined, or a pharmaceutically acceptable salt and/or solvate thereof.

The present invention also provides an N-oxide of a compound as herein defined, or a prodrug or pharmaceutically acceptable salt and/or solvate thereof.

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

The compounds of the present invention can be provided as mixtures of more than one stereoisomer. When provided as a mixture of stereoisomers, one stereoisomer can be present at a purity >90% relative to the remaining stereoisomers. More specifically, when provided as a mixture of stereoisomers, one stereoisomer can be present at a purity >95% relative to the remaining stereoisomers.

It will be understood that substituents may be named as its free unbonded structure (e.g. piperidine) or by its bonded structure (e.g. piperidinyl). No difference is intended.

It will be understood that the compounds of the invention comprise several substituents. When any of these substituents is defined more specifically herein, the substituents/optional substituents to these groups described above also apply, unless stated otherwise. For example, B can be heteroaryl^{a}, which more specifically can be isoquinolinyl. In this case, isoquinolinyl can be optionally substituted in the same manner as “heteroaryl^{a}”.

It will be understood that “alkylene” has two free valencies i.e. it is bivalent, meaning that it is capable of being bonded to twice. For example, when R1 and R2, together with the carbon atom to which they are attached, are linked by alkylene to form a 4-membered saturated ring, the alkylene can be —CH₂CH₂CH₂—.

It will be understood that lines drawn into the ring systems from substituents represent that the indicated bond can be attached to any of the ring atoms capable of being substituted. For example, in formula (I), AW-, X, and R5 (when present) can be attached to any of the ring atoms on Z capable of being substituted.

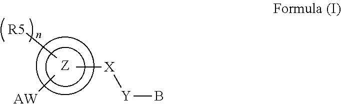

It will be understood that when n is 0, there are no R5 substituents on Z, and only AW- and X substituents are attached to Z.

It will be understood that when Z is 2-pyridone or 4-pyridone, the pyridone can be in any orientation, and substituted at any substitutable ring atoms as allowed by formula (I).

It will be understood that a fused ring system refers to a ring system where two rings in the ring system share two adjacent atoms (i.e one common covalent bond). For example,

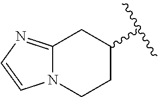

is a fused ring system (specifically a fused bicyclic ring system) which can be considered as an imidazole ring and a piperidine ring sharing a common bond.

It will be understood that a bridged ring system refers to a ring system having two rings sharing three or more atoms. For example,

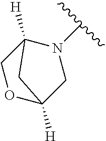 [SMILES: *N1C[C@@H]2C[C@H]1CO2]

is a bridged ring system (specifically a bridged bicyclic ring system) which can be considered as a tetrahydrofuran ring and a pyrrolidine ring joined at a bridge and sharing three common atoms.

It will be understood that a spiro ring system refers to a ring system where two rings in the ring system share one common atom. For example,

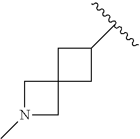 [SMILES: *C1CC2(C1)CN(C)C2]

is a spiro ring system (specifically a spiro bicyclic ring system) which can be considered as a cyclobutane ring and an azetidine ring sharing a common carbon atom.

It will be understood that the ring system A, as defined in formula (I), can be fully saturated, or have any degree of unsaturation. For example, the ring system can be fully saturated, partially unsaturated, aromatic, non-aromatic, or have an aromatic ring bridged, fused or spiro to a non-aromatic ring.

It will be understood that ring system A can contain non-carbon ring members, and that these non-carbon ring members can, where possible, be optionally substituted themselves (as well, or as opposed to the carbon ring members), with the optional substituents included in the definition of A.

It will be understood that, in the instance when Y is attached to B at a carbon atom on the heteroaryl^{a} ring, the attachment of Y to B can be at any carbon on the heteroaryl^{a} ring, so long as the remainder of the ring is still a heteroaryl ring. For example, if B is 7-azaindole, the attachment to Y can be at any of the following ring atoms:

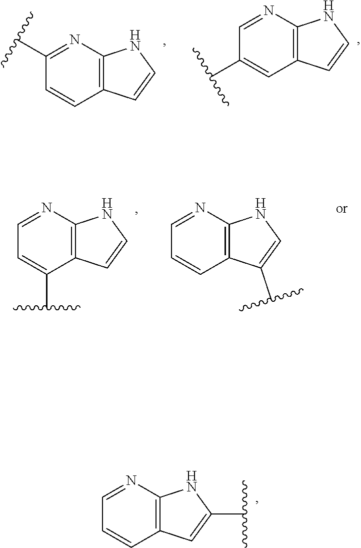 [SMILES: *c1ccc2cc[nH]c2n1 ; *c1cnc2[nH]ccc2c1 ; *c1ccnc2[nH]ccc12 ; *c1c[nH]c2ncccc12 ; *c1cc2cccnc2[nH]1]

but not at a nitrogen ring atom:

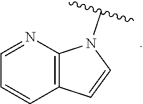 [SMILES: *n1ccc2cccnc21]

It will be understood that, in the instance when Y is attached to B at a carbon atom on the heteroaryl^{a} ring, and the two ring atoms adjacent to the carbon atom on the heteroaryl^{a} ring to which Y attaches are both carbon, these adjacent ring atoms can be, where possible, substituted or unsubstituted as defined in the embodiment or claim. Further, for example, if B is 7-azaindole, the attachment to Y can be at any of the following ring atoms:

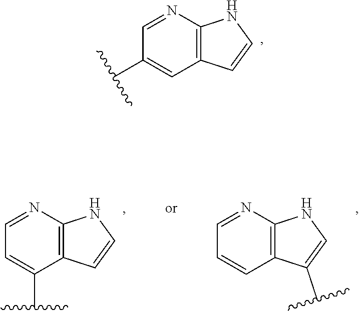 [SMILES: *c1cnc2[nH]ccc2c1 ; *c1ccnc2[nH]ccc12 ; *c1c[nH]c2ncccc12]

but not at the following ring atoms:

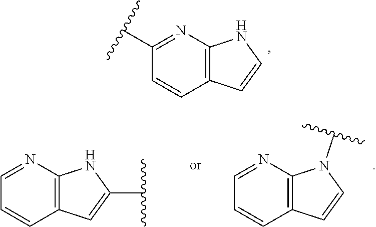 [SMILES: *c1ccc2cc[nH]c2n1 ; *c1cc2cccnc2[nH]1 ; *n1ccc2cccnc21]

It will be understood that when any variable (e.g. alkyl) occurs more than once, its definition on each occurrence is independent of every other occurrence.

It will be understood that combinations of substituents and variables are permissible only if such combinations result in stable compounds.

As used herein the term “bradykinin-mediated angioedema” means hereditary angioedema, and any non-hereditary bradykinin-mediated angioedema. For example, “bradykinin-mediated angioedema” encompasses hereditary angioedema and acute bradykinin-mediated angioedema of unknown origin.

As used herein, the term “hereditary angioedema” means any bradykinin-mediated angioedema caused by an inherited genetic dysfunction, fault, or mutation. As a result, the term “HAE” includes at least HAE type 1, HAE type 2, and normal C1 inhibitor HAE (normal C1-Inh HAE).

Certain preferred sub-formulae of the compounds of formula (I) include compounds of formula (Ia), formula (Ib), formula (Ic), formula (Id), and formula (Ie), as indicated below:

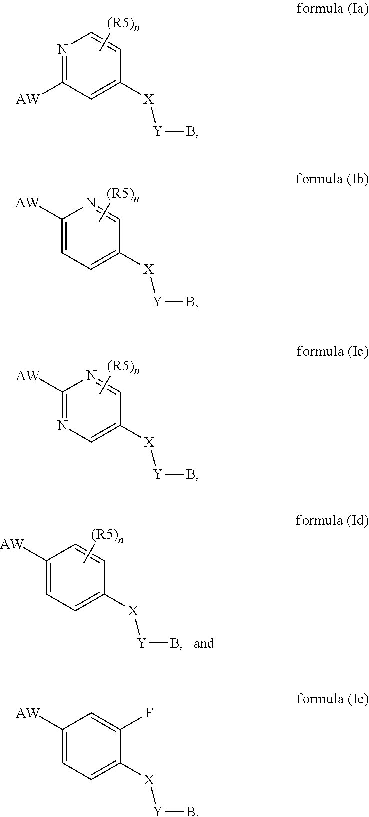

Z can be a 6- or 5-membered heteroaromatic ring containing 1, 2 or 3 ring members independently selected from N, S and O; or phenyl; or Z can be 2-pyridone or 4-pyridone. More specifically, Z can be selected from phenyl, thiophene, furan, pyrrole, pyrazole, imidazole, oxazole, isoxazole, thiazole, isothiazole, triazole, oxadiazole, thiadiazole, pyridine, pyridazine, pyrimidine, pyrazine, triazine, 2-pyridone and 4-pyridone.

Z can be 2-pyridone or 4-pyridone. Z can be 2-pyridone. Z can be 4-pyridone.

Z is a 6- or 5-membered heteroaromatic ring containing 1, 2 or 3 ring members independently selected from N, S and O; or phenyl. More specifically, Z can be selected from phenyl, thiophene, furan, pyrrole, pyrazole, imidazole, oxazole, isoxazole, thiazole, isothiazole, triazole, oxadiazole, thiadiazole, pyridine, pyridazine, pyrimidine, pyrazine and triazine.

Z can be a 6- or 5-membered heteroaromatic ring containing 1 or 2 ring members independently selected from N and S; or phenyl. More specifically, Z can be selected from phenyl, thiophene, pyrrole, pyrazole, imidazole, thiazole, isothiazole, pyridine, pyridazine, pyrimidine and pyrazine.

Z can be a 6-membered heteroaromatic ring containing 1, 2 or 3 ring members independently selected from N; or phenyl; or Z can be 2-pyridone or 4-pyridone. More specifically, Z can be selected from phenyl, pyridine, pyridazine, pyrimidine, pyrazine, triazine, 2-pyridone and 4-pyridone.

Z can be a 6-membered heteroaromatic ring containing 1, 2 or 3 ring members independently selected from N. More specifically, Z can be selected from pyridine, pyridazine, pyrimidine, pyrazine, and triazine.

Z can be a 6- or 5-membered heteroaromatic ring containing 1 or 2 ring members that are N; or phenyl. More specifically, Z can be selected from phenyl, pyrrole, pyrazole, imidazole, pyridine, pyridazine, pyrimidine and pyrazine. Preferably, Z can be selected from pyrazole, phenyl, pyrimidine, pyridine, pyrazine, pyridazine, oxazole, thiophene, and thiazole. Preferably, Z is selected from pyrazole, phenyl, pyrimidine, pyridine and thiazole. More preferably, Z is selected from phenyl, pyrimidine, and pyridine.

Z can be phenyl.

Z can be a 5-membered heteroaromatic ring containing 1, 2 or 3 ring members independently selected from N, S and O. More specifically, Z can be selected from thiophene, furan, pyrrole, pyrazole, imidazole, oxazole, isoxazole, thiazole, isothiazole, triazole, oxadiazole, and thiadiazole.

X can be selected from SO₂ and CR1R2. X can be SO₂. When X is SO₂, Y can be NH. Preferably, X is CR1R2.

R1 can be selected from H, alkyl, alkoxy, OH, halo and NR13R14. R1 can be selected from H and alkyl. R1 can be selected from H, methyl and CH(CH₂F). Preferably, R1 is H.

R2 can be selected from H and small alkyl. R2 can be selected from H and methyl. Preferably, R2 is H.

Alternatively, R1 and R2, together with the carbon atom to which they are attached, can be linked by alkylene to form a 3-, 4-, or 5-membered saturated ring. Preferably, R1 and R2, together with the carbon atom to which they are attached, are linked by alkylene to form a 3- or 4-membered saturated ring.

Y can be selected from NR12, O, and CR3R4. Y can be selected from NH, N(alkyl), N(cycloalkyl), O, CH₂, CH(alkyl) and C(alkyl)(alkyl). Y can be selected from NH, N(CH₃), O, and CH₂. Y can be selected from NH and N(CH₃). Preferably Y is NH.

Alternatively, X can be CR1R2 and Y can be CR3R4, and R1 and R3, together with the carbon atom to which R1 is attached and the carbon atom to which R3 is attached, can be linked by alkylene to form a 3-, 4-, or 5-membered saturated ring. For example, X can be CR1R2 and Y can be CR3R4, and R1 and R3, together with the carbon atom to which R1 is attached and the carbon atom to which R3 is attached, can be linked by alkylene to form a 3-membered saturated ring. For example, X can be CR1R2 and Y can be CR3R4, and R1 and R3, together with the carbon atom to which R1 is attached and the carbon atom to which R3 is attached, can be linked by alkylene to form a 4-membered saturated ring. For example, X can be CR1R2 and Y can be CR3R4, and R1 and R3, together with the carbon atom to which R1 is attached and the carbon atom to which R3 is attached, can be linked by alkylene to form a 5-membered saturated ring.

R3 and R4 can be independently selected from H and alkyl. Preferably at least one of R3 and R4 is H. More preferably, both R3 and R4 are H.

Alternatively, X can be CR1R2 and Y can be NR12, and R1 and R12, together with the carbon atom to which R1 is attached and the nitrogen atom to which R12 is attached, can be linked by alkylene to form a 3-, 4-, or 5-membered saturated heterocycle. For example, X can be CR1R2 and Y can be NR12, and R1 and R12, together with the carbon atom to which R1 is attached and the nitrogen atom to which R12 is attached, can be linked by alkylene to form a 3-membered saturated heterocycle. For example, X can be CR1R2 and Y can be NR12, and R1 and R12, together with the carbon atom to which R1 is attached and the nitrogen atom to which R12 is attached, can be linked by alkylene to form a 4-membered saturated heterocycle. For example, X can be CR1R2 and Y can be NR12, and R1 and R12, together with the carbon atom to which R1 is attached and the nitrogen atom to which R12 is attached, can be linked by alkylene to form a 5-membered saturated heterocycle.

Preferably, Z is selected from pyrazole, phenyl, pyrimidine, pyridine, pyrazine, pyridazine, oxazole, thiophene, and thiazole; X is CR1R2; R1 is H; R2 is H and Y is NH. More specifically, Z is selected from phenyl, pyrimidine, and pyridine; X is CR1R2; R1 is H; R2 is H and Y is NH.

Preferably, Z is selected from pyrazole, phenyl, pyrimidine, pyridine and thiazole; X is CR1R2; R1 is H; R2 is H and Y is NH. More specifically, Z is selected from phenyl, pyrimidine, and pyridine; X is CR1R2; R1 is H; R2 is H and Y is NH.

Preferably, the compound of formula (I) is a compound of formula (Ia), formula (Ib), formula (Ic), formula (Id), or formula (Ie); X is CR1R2; R1 is H; R2 is H and Y is NH.

B can be selected from:
(i) heteroaryl^{a};
(ii) aryl;
(iii) a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃; and
(iv) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring.

B can be selected from:
(i) heteroaryl^{a};
(ii) a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃; and
(iii) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring.

B can be selected from:
(i) heteroaryl^{a};
(ii) aryl; and
(iii) a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃.

Specifically, B is selected from:
(i) heteroaryl^{a}; and
(ii) a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃.

Preferably, B is heteroaryl^{a}. Preferably, when B is heteroaryl^{a}, B is preferably substituted with NH₂, and optionally substituted with 1 or 2 further substituents as for heteroaryl^{a}

When B is heteroaryl^{a}, B can be a 5, 6, 9 or 10 membered mono- or bi-cyclic aromatic ring, containing, where possible, 1, 2, 3 or 4 ring members independently selected from N, NR12, S and O; wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be a 5, 6, 9 or 10 membered mono- or bi-cyclic aromatic ring, containing, where possible, 1, 2, 3 or 4 ring members independently selected from N, NR12, S and O; wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be a 9 or 10 membered bi-cyclic aromatic ring, containing, where possible, 1, 2, 3 or 4 ring members independently selected from N, NR12, S and O, optionally substituted as for heteroaryl^{a}. B can be a 9 or 10 membered bi-cyclic aromatic ring, containing, where possible, 1, 2, 3 or 4 ring members independently selected from N, NR12, S and O, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be a 9 or 10 membered bi-cyclic aromatic ring, containing, where possible, 1, 2, 3 or 4 ring members independently selected from N, NR12, S and O, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be a 9 or 10 membered bi-cyclic aromatic ring, containing, where possible, 1 or 2 ring members independently selected from N, NR12, S and O, optionally substituted as for heteroaryl^{a}. B can be a 9 or 10 membered bi-cyclic aromatic ring, containing, where possible, 1 or 2 ring members independently selected from N, NR12, S and O, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be a 9 or 10 membered bi-cyclic aromatic ring, containing, where possible, 1 or 2 ring members independently selected from N, NR12, S and O, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be a 9 or 10 membered bi-cyclic aromatic ring, containing, where possible, 1 or 2 ring members independently selected from N and NR12, optionally substituted as for heteroaryl^{a}. B can be a 9 or 10 membered bi-cyclic aromatic ring, containing, where possible, 1 or 2 ring members independently selected from N and NR12, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be a 9 or 10 membered bi-cyclic aromatic ring, containing, where possible, 1 or 2 ring members independently selected from N and NR12, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, Y is preferably attached to B at a carbon atom on the heteroaryl^{a} ring. Specifically, when B is heteroaryl^{a}, Y is preferably attached to B at a carbon atom on the heteroaryl^{a} ring, and the two ring atoms adjacent to the carbon atom on the heteroaryl^{a} ring to which Y attaches are both carbon. When B is heteroaryl^{a}, B is preferably selected from isoquinolinyl

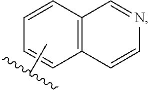

optionally substituted as for heteroaryl^{a}; 6-azaindolyl

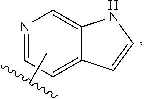

optionally substituted as for heteroaryl^{a}; 7-azaindolyl

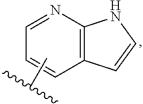

optionally substituted as for heteroaryl^{a}; and pyridyl

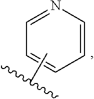

optionally substituted as for heteroaryl^{a}. B can be isoquinolinyl

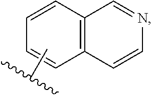

optionally substituted as for heteroaryl^{a}. B can be 6-azaindolyl

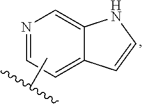

optionally substituted as for heteroaryl^{a}. B can be 7-azaindolyl

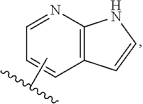

optionally substituted as for heteroaryl^{a}. B can be pyridyl

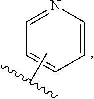

optionally substituted as for heteroaryl^{a}.

More specifically, B is selected from isoquinolinyl, selected from

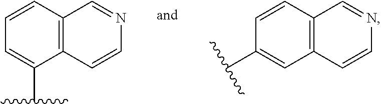 [SMILES: *c1cccc2cnccc12 ; *c1ccc2cnccc2c1]

optionally substituted as for heteroaryl^{a}; 6-azaindolyl

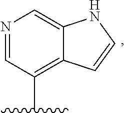 [SMILES: *c1cncc2[nH]ccc12]

optionally substituted as for heteroaryl^{a}; 7-azaindolyl

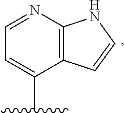 [SMILES: *c1ccnc2[nH]ccc12]

optionally substituted as for heteroaryl^{a}; and pyridyl

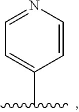 [SMILES: *c1ccncc1]

optionally substituted as for heteroaryl^{a}. B can be isoquinolinyl, selected from

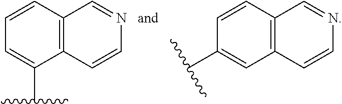 [SMILES: *c1cccc2cnccc12 ; "and" ; *c1ccc2cnccc2c1]

B can be 6-azaindolyl

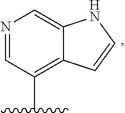 [SMILES: *c1cncc2[nH]ccc12]

optionally substituted as for heteroaryl^{a}. B can be 7-azaindolyl

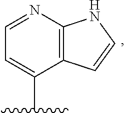 [SMILES: *c1ccnc2[nH]ccc12]

optionally substituted as for heteroaryl^{a}. B can be pyridyl

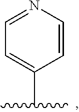 [SMILES: *c1ccncc1]

optionally substituted as for heteroaryl^{a}.

More specifically, B is selected from: isoquinolinyl

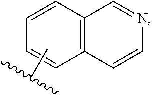

substituted with NH₂, optionally further substituted with 1 or 2 substituents as for heteroaryl^{a}; 6-azaindolyl

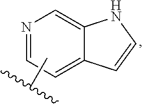

optionally substituted as for heteroaryl^{a}; 7-azaindolyl

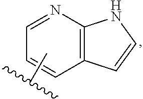

optionally substituted as for heteroaryl^{a}; and pyridyl

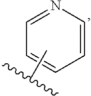

optionally substituted as for heteroaryl^{a}. B can be isoquinolinyl

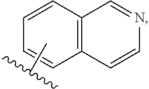

substituted with NH₂, optionally further substituted with 1 or 2 substituents as for heteroaryl^{a}. B can be 6-azaindolyl,

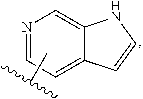

optionally substituted as for heteroaryl^{a}. B can be 7-azaindolyl

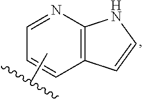

optionally substituted as for heteroaryl^{a}. B can be pyridyl

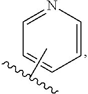

optionally substituted as for heteroaryl^{a}.

More specifically, B is selected from isoquinolinyl, selected from

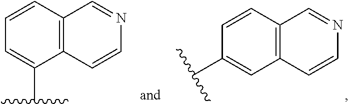 [SMILES: *c1cccc2cnccc12 ; *c1ccc2cnccc2c1]

substituted with NH₂, optionally further substituted with 1 or 2 substituents as for heteroaryl^{a}; 6-azaindolyl

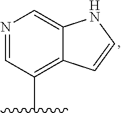 [SMILES: *c1cncc2[nH]ccc12]

optionally substituted as for heteroaryl^{a}; 7-azaindolyl

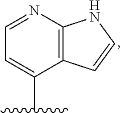 [SMILES: *c1ccnc2[nH]ccc12]

optionally substituted as for heteroaryl^{a}; and pyridyl

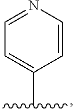 [SMILES: *c1ccncc1]

optionally substituted as for heteroaryl^{a}. B can be isoquinolinyl, selected from

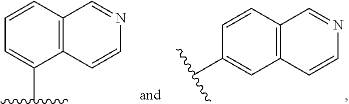 [SMILES: *c1cccc2cnccc12 ; *c1ccc2cnccc2c1]

and substituted with NH₂, optionally further substituted with 1 or 2 substituents as for heteroaryl^{a}. B can be 6-azaindolyl

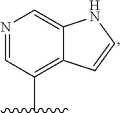 [SMILES: *c1cncc2[nH]ccc12]

optionally substituted as for heteroaryl^{a}. B can be 7-azaindolyl

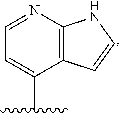 [SMILES: *c1ccnc2[nH]ccc12]

optionally substituted as for heteroaryl^{a}. B can be pyridyl

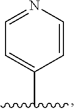 [SMILES: *c1ccncc1]

optionally substituted as for heteroaryl^{a}.

Yet more specifically, B is selected from: isoquinolinyl, substituted with NH₂ at the 1-position

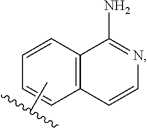

optionally further substituted with 1 or 2 substituents as for heteroaryl^{a}; 6-azaindolyl

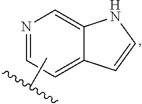

optionally substituted as for heteroaryl^{a}; 7-azaindolyl

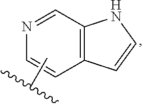

optionally substituted as for heteroaryl^{a}; and pyridyl

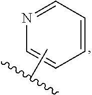

optionally substituted as for heteroaryl^{a}. B can be isoquinolinyl, substituted with NH₂ at the 1-position

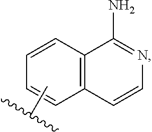

optionally further substituted with 1 or 2 substituents as for heteroaryl^{a}. B can be 6-azaindolyl

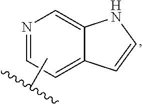

optionally substituted as for heteroaryl^{a}. B can be 7-azaindolyl

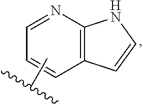

optionally substituted as for heteroaryl^{a}. B can be pyridyl

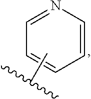

optionally substituted as for heteroaryl^{a}.

Preferably, when B is heteroaryl^{a}, B is selected from: isoquinolinyl, substituted with NH₂ at the 1-position, selected from

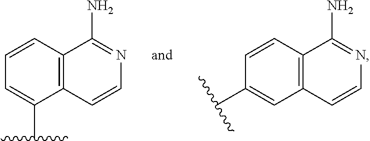 [SMILES: *c1cccc2c(N)nccc12 ; *c1ccc2c(N)nccc2c1]

optionally further substituted with 1 or 2 substituents as for heteroaryl^{a}; 6-azaindolyl

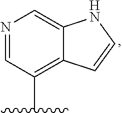 [SMILES: *c1cncc2[nH]ccc12]

optionally substituted as for heteroaryl^{a}; 7-azaindolyl

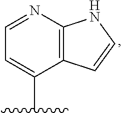 [SMILES: *c1ccnc2[nH]ccc12]

optionally substituted as for heteroaryl^{a}; and pyridyl

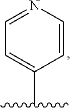 [SMILES: *c1ccncc1]

optionally substituted as for heteroaryl^{a}.

Specifically, B can be isoquinolinyl, substituted with NH₂ at the 1-position, selected from

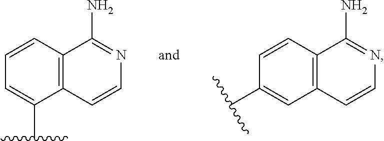 [SMILES: *c1cccc2c(N)nccc12 ; *c1ccc2c(N)nccc2c1]

optionally further substituted with 1 or 2 substituents as for heteroaryl^{a}. B can be isoquinolinyl, substituted with NH₂ at the 1-position

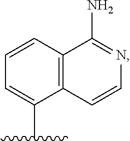 [SMILES: *c1cccc2c(N)nccc12]

optionally further substituted with 1 or 2 substituents as for heteroaryl^{a}. B can be isoquinolinyl, substituted with NH₂ at the 1-position

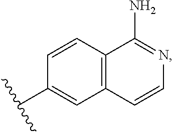 [SMILES: *c1ccc2c(N)nccc2c1]

optionally further substituted with 1 or 2 substituents as for heteroaryl^{a}. B can be 6-azaindolyl

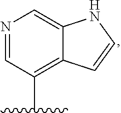 [SMILES: *c1cncc2[nH]ccc12]

optionally substituted as for heteroaryl^{a}. B can be 7-azaindolyl

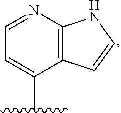 [SMILES: *c1ccnc2[nH]ccc12]

optionally substituted as for heteroaryl^{a}. B can be pyridyl

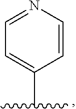 [SMILES: *c1ccncc1]

optionally substituted as for heteroaryl^{a}.

When B is heteroaryl^{a}, B is preferably isoquinolinyl, optionally substituted as for heteroaryl^{a}. B is preferably isoquinolinyl optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B is preferably isoquinolinyl optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is isoquinolinyl, B can be selected from

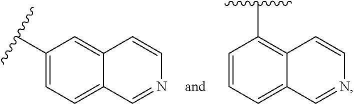 [SMILES: *c1ccc2cnccc2c1 ; *c1cccc2cnccc12]

and, optionally substituted as for heteroaryl^{a}. B can be selected from

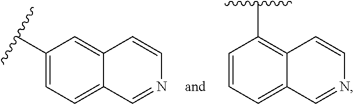 [SMILES: *c1ccc2cnccc2c1 ; *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be selected from and

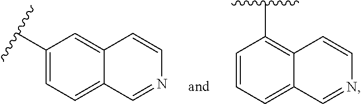 [SMILES: *c1ccc2cnccc2c1 ; *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is isoquinolinyl, B can be

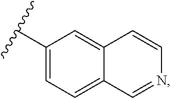 [SMILES: *c1ccc2cnccc2c1]

optionally substituted as for heteroaryl^{a}. B can be

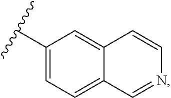 [SMILES: *c1ccc2cnccc2c1]

optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be

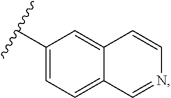 [SMILES: *c1ccc2cnccc2c1]

optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is isoquinolinyl, B can be

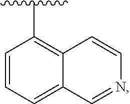 [SMILES: *c1cccc2cnccc12]

optionally substituted as for heteroaryl^{a}. B can be

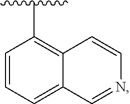 [SMILES: *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be

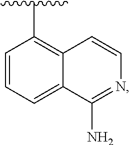 [SMILES: *c1cccc2c(N)nccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B is preferably isoquinolinyl, substituted with NH₂, and optionally substituted with 1 or 2 further substituents as for heteroaryl^{a}. B is preferably isoquinolinyl, substituted with NH₂, and optionally substituted with 1, or 2 further substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B is preferably isoquinolinyl, substituted with NH₂, and optionally substituted with 1, or 2 further substituents independently selected from alkyl, alkoxy, OH, OCF₃, halo, aryl^{b}, —(CH₂)₀₋₃—NR13R14, heteroaryl^{b} and CF₃.

When B is isoquinolinyl, substituted with NH₂, B can be selected from

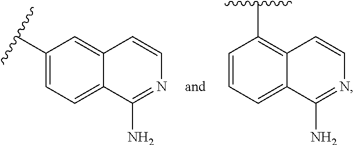 [SMILES: *c1ccc2c(N)nccc2c1 ; *c1cccc2c(N)nccc12]

optionally substituted with 1 or 2 further substituents as for heteroaryl^{a}. B can be selected from

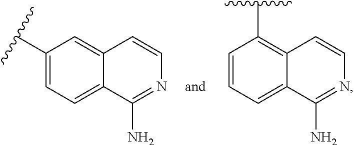 [SMILES: *c1ccc2c(N)nccc2c1 ; *c1cccc2c(N)nccc12]

optionally substituted with 1, or 2 further substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be selected from

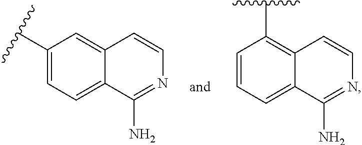 [SMILES: *c1ccc2c(N)nccc2c1 ; *c1cccc2c(N)nccc12]

optionally substituted with 1, or 2 further substituents independently selected from alkyl, alkoxy, OH, OCF₃, halo, aryl^{b}, —(CH₂)₀₋₃—NR13R14, heteroaryl^{b} and CF₃.

When B is isoquinolinyl, substituted with NH₂, B can be

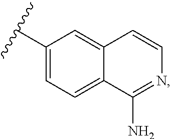 [SMILES: *c1ccc2c(N)nccc2c1]

optionally substituted with 1 or 2 further substituents as for heteroaryl^{a}. B can be

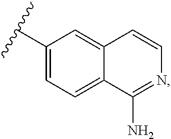 [SMILES: *c1ccc2c(N)nccc2c1]

optionally substituted with 1, or 2 further substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be

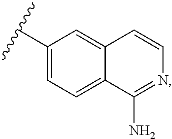 [SMILES: *c1ccc2c(N)nccc2c1]

optionally substituted with 1, or 2 further substituents independently selected from alkyl, alkoxy, OH, OCF₃, halo, aryl^{b}, —(CH₂)₀₋₃—NR13R14, heteroaryl^{b} and CF₃.

When B is isoquinolinyl, substituted with NH₂, B can be

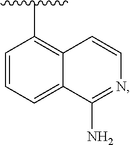 [SMILES: *c1cccc2c(N)nccc12]

optionally substituted with 1 or 2 further substituents as for heteroaryl^{a}. B can be

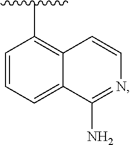 [SMILES: *c1cccc2c(N)nccc12]

optionally substituted with 1, or 2 further substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be

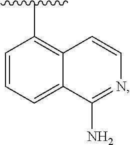 [SMILES: *c1cccc2c(N)nccc12]

optionally substituted with 1, or 2 further substituents independently selected from alkyl, alkoxy, OH, OCF₃, halo, aryl^{b}, —(CH₂)₀₋₃—NR13R14, heteroaryl^{b} and CF₃.

When B is isoquinolinyl, substituted with NH₂, B can be selected from

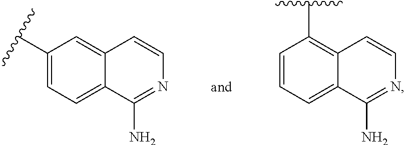 [SMILES: *c1ccc2c(N)nccc2c1 ; "and" ; *c1cccc2c(N)nccc12]

optionally substituted with a further substituent selected from halo.

When B is isoquinolinyl, substituted with NH₂, B can be

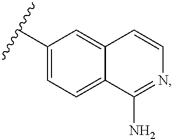 [SMILES: *c1ccc2c(N)nccc2c1]

optionally substituted with a further substituent selected from halo.

When B is isoquinolinyl, substituted with NH₂, B can be

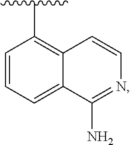 [SMILES: *c1cccc2c(N)nccc12]

optionally substituted with a further substituent selected from halo.

When B isoquinolinyl, substituted with NH₂, B can be selected from

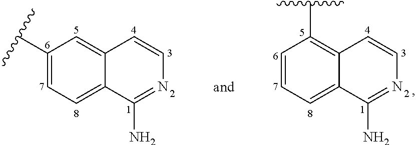 [SMILES: *c1ccc2c(N)nccc2c1 ; "and" ; *c1cccc2c(N)nccc12]

optionally substituted with a further substituent selected from halo at the carbon marked as 4.

When B is isoquinolinyl, substituted with NH₂, B can be

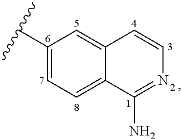 [SMILES: *c1ccc2c(N)nccc2c1]

optionally substituted with a further substituent selected from halo at the carbon marked as 4.

When B is isoquinolinyl, substituted with NH₂, B can be

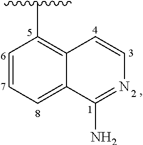 [SMILES: *c1cccc2c(N)nccc12]

optionally substituted with a further substituent selected from halo, at the carbon marked as 4.

Preferably, B is selected from:

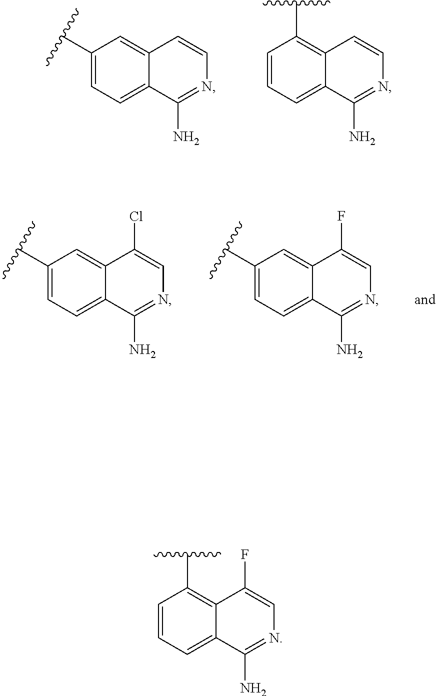 [SMILES: *c1ccc2c(N)nccc2c1 ; *c1cccc2c(N)nccc12 ; *c1ccc2c(N)ncc(Cl)c2c1 ; *c1ccc2c(N)ncc(F)c2c1 ; "and" ; *c1cccc2c(N)ncc(F)c12]

When B is heteroaryl^{a}, B can be a 5, 6, 9 or 10 membered mono- or bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃.

When B is heteroaryl^{a}, B can be a 9 or 10 membered bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃

When B is heteroaryl^{a}, B can be a 5, 6, 9 or 10 membered mono- or bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃.

When B is heteroaryl^{a}, B can be a 9 or 10 membered bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃.

When B is heteroaryl^{a}, B can be a 5, 6, 9 or 10 membered mono- or bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be a 9 or 10 membered bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be quinolinyl or isoquinolinyl which is substituted with 1, 2 or 3 substituents independently selected from alkyl, alkoxy, OH, OCF₃, CN, halo, aryl^{b}, —(CH₂)₀₋₃—NR13R14, heteroaryl^{b}, —C(═O)OR12, —C(═O)NR13R14 and CF₃. B can be quinolinyl or isoquinolinyl which is substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be quinolinyl or isoquinolinyl which is substituted with 1, 2 or 3 substituents independently selected from alkyl, alkoxy, OH, OCF₃, halo, aryl^{b}, —(CH₂)₀₋₃—NR13R14, heteroaryl^{b} and CF₃. When B is heteroaryl^{a}, B can be isoquinolinyl which is substituted with 1, 2 or 3 substituents independently selected from alkyl, alkoxy, OH, OCF₃, CN, halo, aryl^{b}, —(CH₂)₀₋₃—NR13R14, heteroaryl^{b}, —C(═O)OR12, —C(═O)NR13R14 and CF₃. B can be isoquinolinyl which is substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be isoquinolinyl which is substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be isoquinolinyl substituted with 1, 2 or 3 substituents independently selected from alkyl, alkoxy, OH, OCF₃, halo, CN, aryl^{b}, —(CH₂)₁₋₃—NR13R14, heteroaryl^{b}, —C(═O)OR12, —C(═O)NR13R14 and CF₃.

When B is heteroaryl^{a}, B can be isoquinolinyl substituted with 1, 2 or 3 substituents independently selected from alkoxy.

When B is heteroaryl^{a}, B can be isoquinolinyl substituted with 1, 2 or 3 substituents selected from —OMe.

When B is heteroaryl^{a} B can be isoquinolinyl substituted with —OMe. B can be selected from:

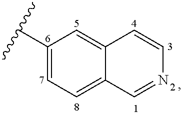 [SMILES: *c1ccc2cnccc2c1]

substituted with —OMe at one of the carbons marked as 3, 4, 5, 7 or 8; and

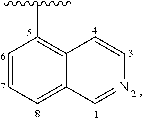 [SMILES: *c1cccc2cnccc12]

substituted with —OMe at one of the carbons marked as 3, 4, 6, 7 or 8. B can be selected from

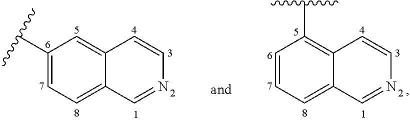 [SMILES: *c1ccc2cnccc2c1 ; *c1cccc2cnccc12]

substituted with —OMe at the carbon marked as 8. B can be

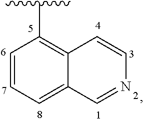 [SMILES: *c1cccc2cnccc12]

substituted with —OMe at one of the carbons marked as 3, 4, 6, 7 or 8. B can be

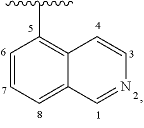 [SMILES: *c1cccc2cnccc12]

substituted with —OMe at the carbon marked as 8. B can be

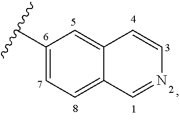 [SMILES: *c1ccc2cnccc2c1]

substituted with —OMe at one of the carbons marked as 3, 4, 5, 7 or 8. B can be

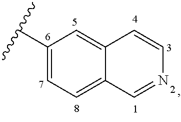 [SMILES: *c1ccc2cnccc2c1]

substituted with —OMe at the carbon marked as 8.

When B is heteroaryl^{a}, B can be isoquinolinyl substituted with -Me. B can be selected from:

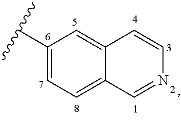 [SMILES: *c1ccc2cnccc2c1]

substituted with -Me at one of the carbons marked as 3, 4, 5, 7 or 8; and

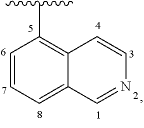 [SMILES: *c1cccc2cnccc12]

substituted with -Me at one of the carbons marked as 3, 4, 6, 7 or 8. B can be elected from

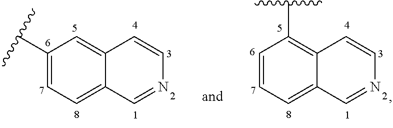 [SMILES: *c1ccc2cnccc2c1 ; *c1cccc2cnccc12]

substituted with -Me at the carbon marked as 8. B can be

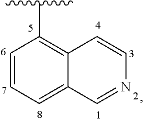 [SMILES: *c1cccc2cnccc12]

substituted with -Me at one of the carbons marked as 3, 4, 6, 7 or 8. B can be

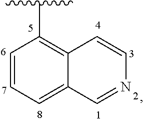 [SMILES: *c1cccc2cnccc12]

substituted with -Me at the carbon marked as 8. B can be

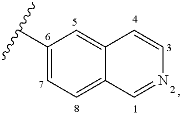 [SMILES: *c1ccc2cnccc2c1]

substituted with -Me at one of the carbons marked as 3, 4, 5, 7 or 8. B can be

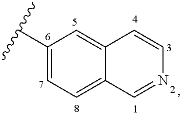 [SMILES: *c1ccc2cnccc2c1]

substituted with -Me at the carbon marked as 8.

When B is heteroaryl^{a}, B can be a 9-membered, bi-cyclic aromatic ring containing 1 or 2 ring members independently selected from N, NR12, S and O; wherein B may be optionally substituted as for heteroaryl^{a}.

When B is heteroaryl^{a}, B can be a 9-membered, bi-cyclic aromatic ring containing 1 or 2 ring members independently selected from N, NR12, S and O; wherein B is substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃; wherein the substituents on B are attached to carbon ring members only.

Preferably, when B is heteroaryl^{a}, the optional substituents on B are, where possible, independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃.

When B is heteroaryl^{a}, B can be selected from

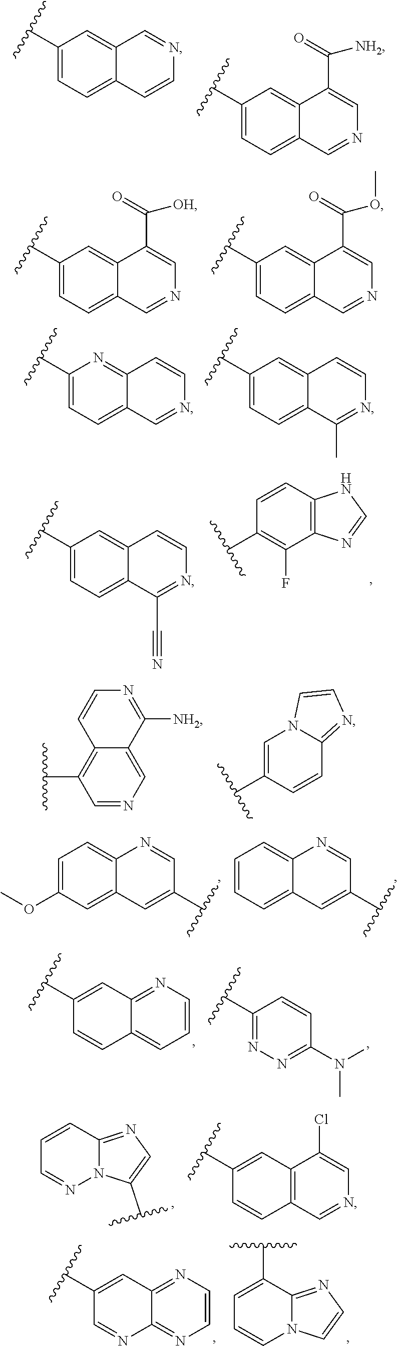

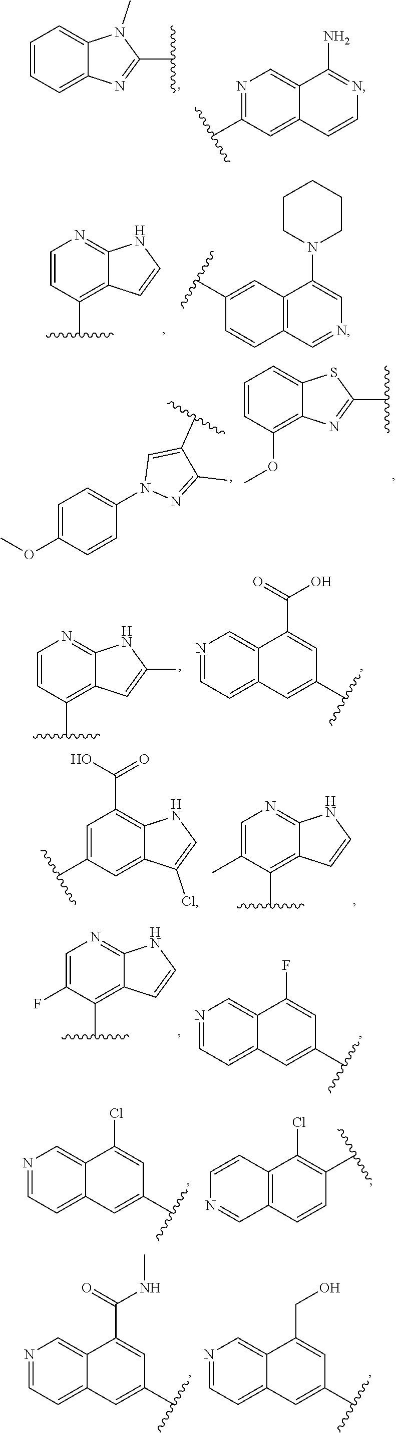

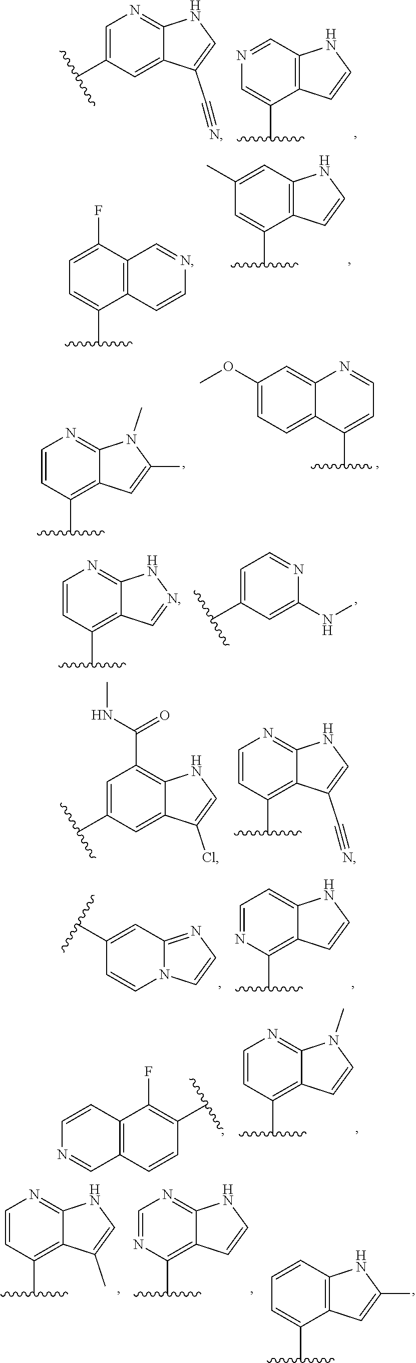

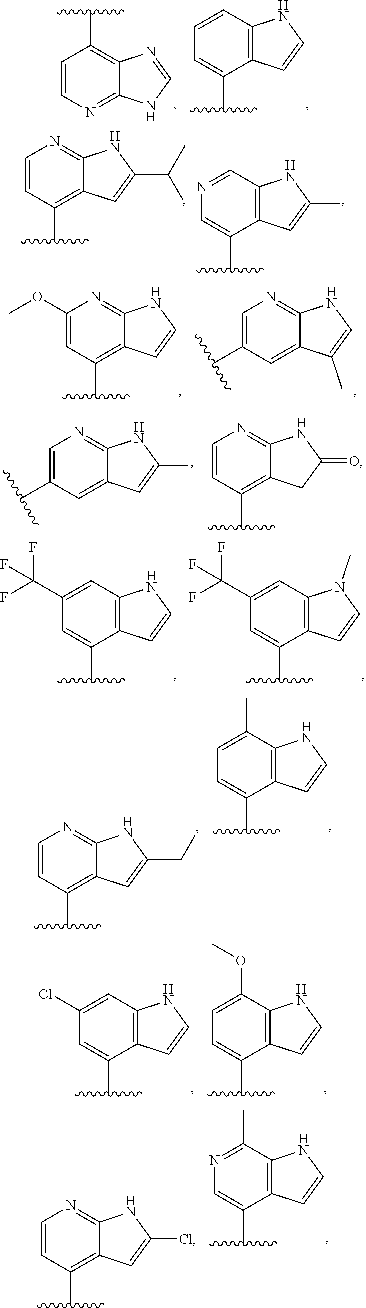

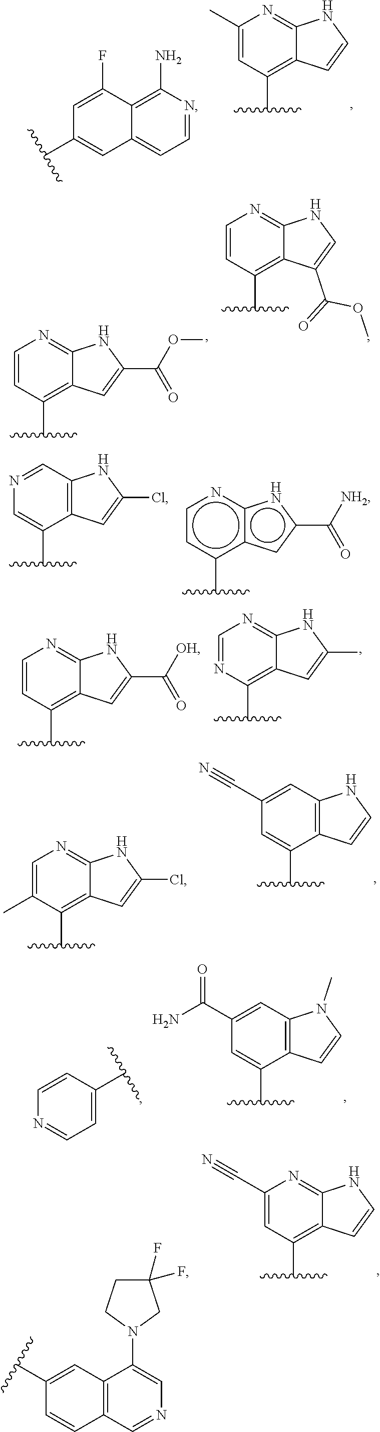

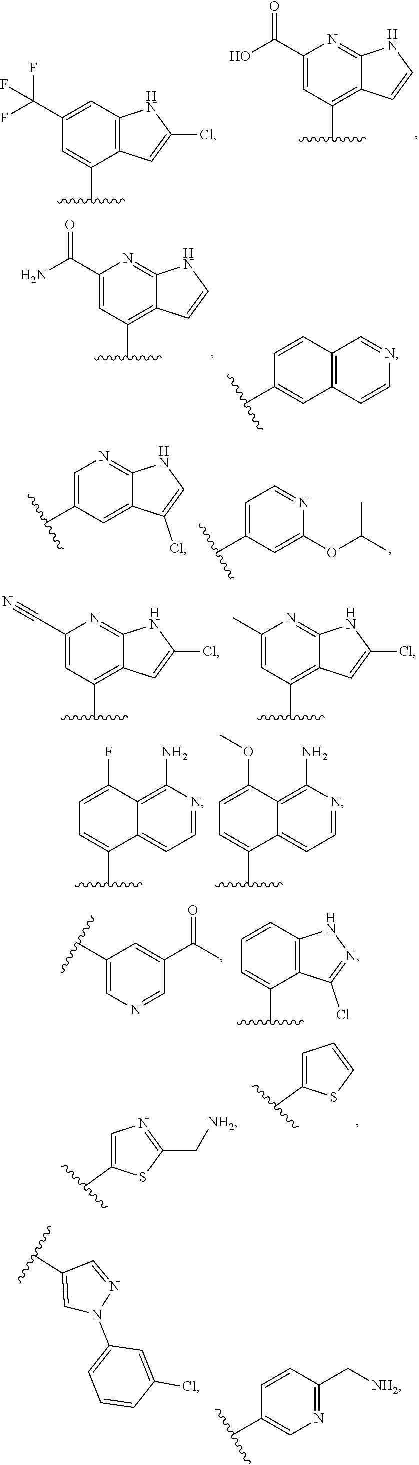

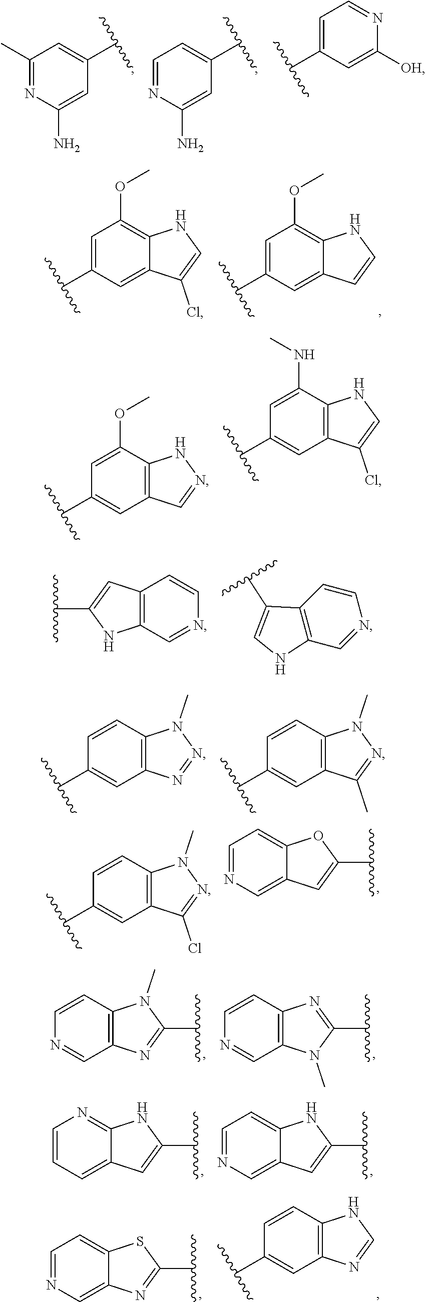

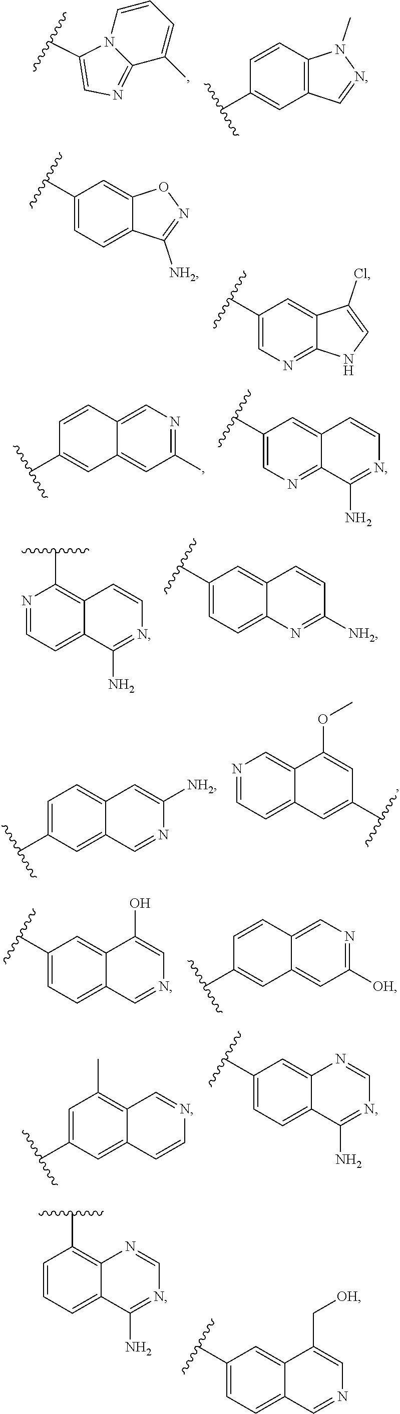

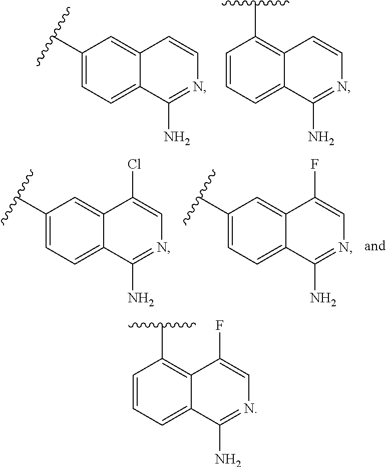

When B is heteroaryl^{a}, B can be selected from

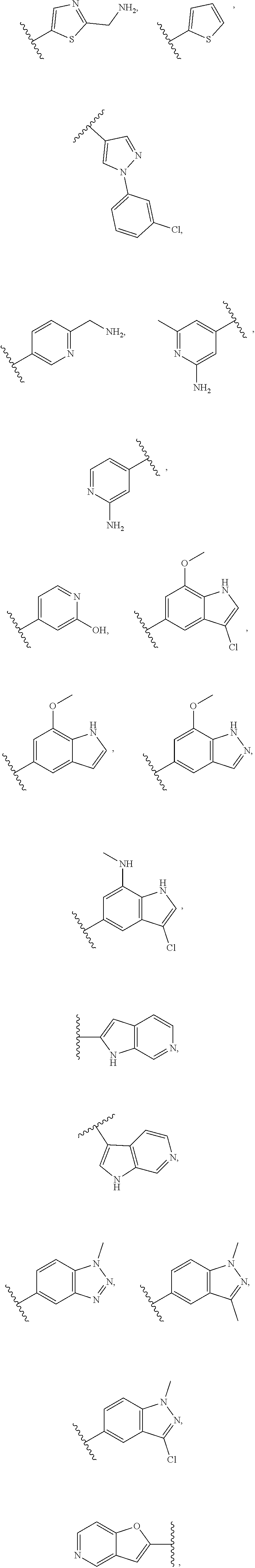

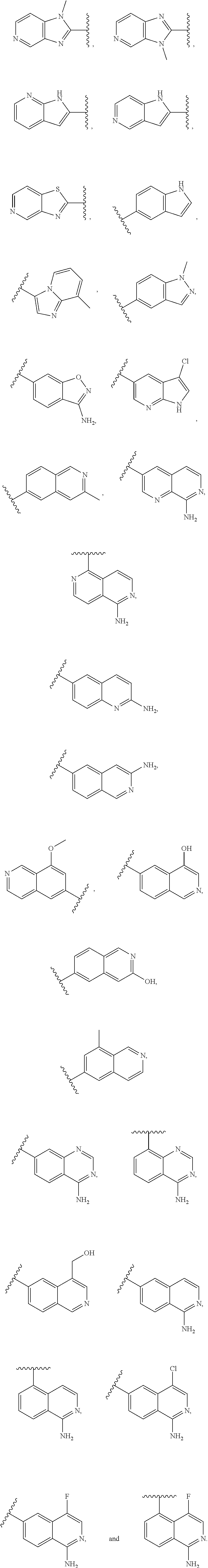

Preferably B is selected from:

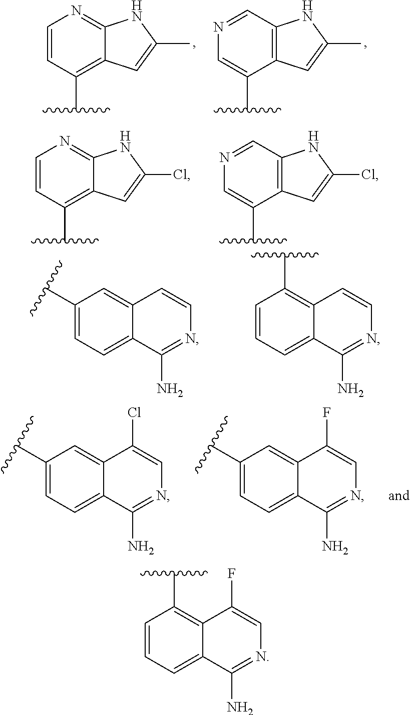

Preferably, B is selected from:

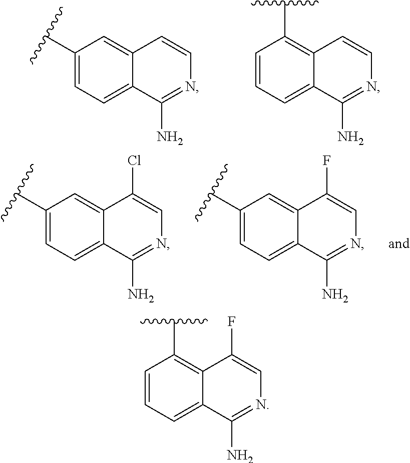

B can be aryl. B can be phenyl or naphthyl, wherein B may be optionally substituted as for aryl. When B is aryl, preferably B is phenyl, wherein B may be optionally substituted as for aryl.

B can be selected from:

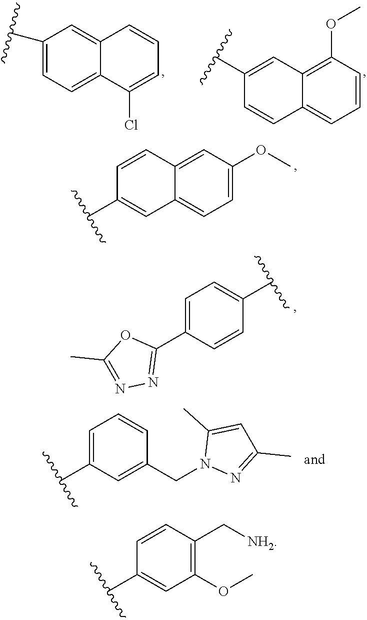

B can be selected from:

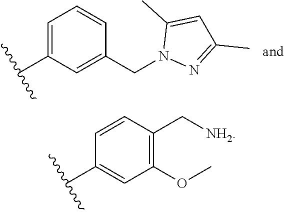

B can be a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃.

B can be pyrrolidine which may be optionally substituted by 1, 2 or 3 substituents independently selected from alkyl, alkoxy, aryl^{b}, OH, OCF₃, halo, oxo, CN, and CF₃.

B can be pyrrolidine which may be optionally substituted with 1 aryl^{b}.

B can be pyridone which is unsaturated with 2 double bonds, which may be optionally substituted by 1, 2 or 3 substituents independently selected from alkyl, alkoxy, aryl^{b}, OH, OCF₃, halo, oxo, CN, and CF₃.

B can be pyridone which is unsaturated with 2 double bonds, substituted by two alkyl groups.

B can be selected from:

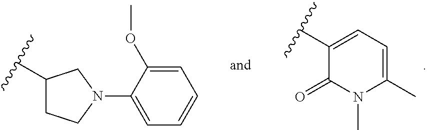

B can be a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring.

B can be selected from:

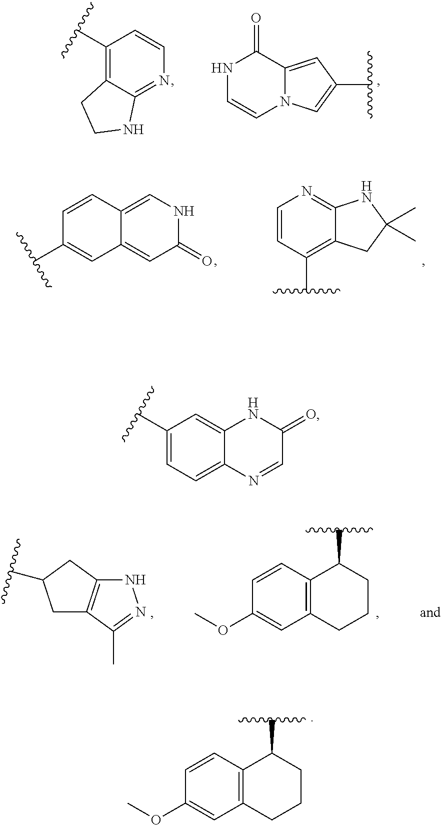

B can be selected from:

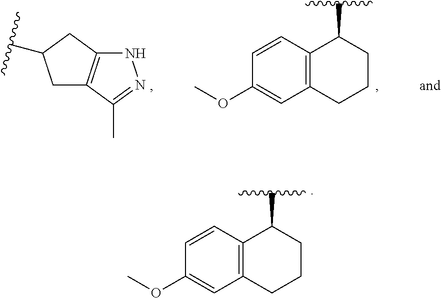

Preferably, Z is selected from pyrazole, phenyl, pyrimidine, pyridine, pyrazine, pyridazine, oxazole, thiophene, and thiazole; X is CR1R2; R1 is H; R2 is H; Y is NH; and B is heteroaryl^{a}.

Preferably, Z is selected from pyrazole, phenyl, pyrimidine, pyridine and thiazole; X is CR1R2; R1 is H; R2 is H; Y is NH; and B is heteroaryl^{a}. More specifically, Z is selected from phenyl, pyrimidine, and pyridine; X is CR1R2; R1 is H; R2 is H; Y is NH; and B is heteroaryl^{a}.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; and B is selected from: isoquinolinyl, substituted with NH₂ at the 1-position, selected from

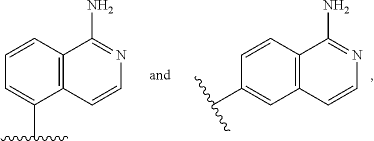 [SMILES: *c1cccc2c(N)nccc12 ; *c1ccc2c(N)nccc2c1]

optionally further substituted with 1 or 2 substituents as for heteroaryl^{a}; 6-azaindolyl

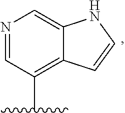 [SMILES: *c1cncc2[nH]ccc12]

optionally substituted as for heteroaryl^{a}; 7-azaindolyl

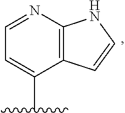 [SMILES: *c1ccnc2[nH]ccc12]

optionally substituted as for heteroaryl^{a}; and pyridyl

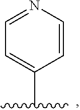 [SMILES: *c1ccncc1]

optionally substituted as for heteroaryl^{a}.

More preferably, Z is selected from pyrazole, phenyl, pyrimidine, pyridine and thiazole; X is CR1R2; R1 is H; R2 is H; Y is NH; and B is selected from: isoquinolinyl, substituted with NH₂ at the 1-position, selected from

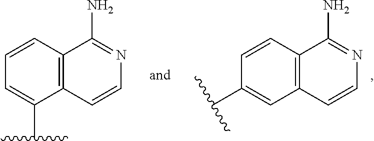 [SMILES: *c1cccc2c(N)nccc12 ; *c1ccc2c(N)nccc2c1]

optionally further substituted with 1 or 2 substituents as for heteroaryl^{a}; 6-azaindolyl

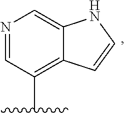 [SMILES: *c1cncc2[nH]ccc12]

optionally substituted as for heteroaryl^{a}; 7-azaindolyl

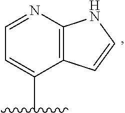 [SMILES: *c1ccnc2[nH]ccc12]

optionally substituted as for heteroaryl^{a}; and pyridyl

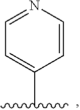 [SMILES: *c1ccncc1]

optionally substituted as for heteroaryl^{a}. More specifically, Z is selected from phenyl, pyrimidine, and pyridine; X is CR1R2; R1 is H; R2 is H; Y is NH; and B is selected from: isoquinolinyl, substituted with NH₂ at the 1-position, selected from

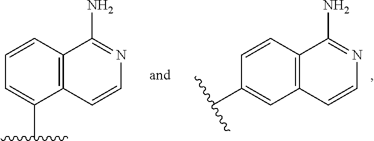 [SMILES: *c1cccc2c(N)nccc12 ; *c1ccc2c(N)nccc2c1]

optionally further substituted with 1 or 2 substituents as for heteroaryl^{a}; 6-azaindolyl

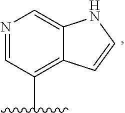 [SMILES: *c1cncc2[nH]ccc12]

optionally substituted as for heteroaryl^{a}; 7-azaindolyl

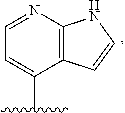 [SMILES: *c1ccnc2[nH]ccc12]

optionally substituted as for heteroaryl^{a}; and pyridyl

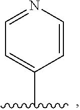 [SMILES: *c1ccncc1]

optionally substituted as for heteroaryl^{a}.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; and B is isoquinolinyl, optionally substituted as for heteroaryl^{a}.

More preferably, Z is selected from pyrazole, phenyl, pyrimidine, pyridine and thiazole; X is CR1R2; R1 is H; R2 is H; Y is NH; and B is isoquinolinyl, optionally substituted as for heteroaryl^{a}. More specifically, Z is selected from phenyl, pyrimidine, and pyridine; X is CR1R2; R1 is H; R2 is H; Y is NH; and B is isoquinolinyl, optionally substituted as for heteroaryl^{a}.

Yet more preferably Z is selected from pyrazole, phenyl, pyrimidine, pyridine, pyrazine, pyridazine, oxazole, thiophene, and thiazole; X is CR1R2; R1 is H; R2 is H; Y is NH; and B is isoquinolinyl, substituted with NH₂, and optionally substituted with 1 or 2 further substituents as for heteroaryl^{a}.

Yet more preferably Z is selected from pyrazole, phenyl, pyrimidine, pyridine and thiazole; X is CR1R2; R1 is H; R2 is H; Y is NH; and B is isoquinolinyl, substituted with NH₂, and optionally substituted with 1 or 2 further substituents as for heteroaryl^{a}. More specifically, Z is selected from phenyl, pyrimidine, and pyridine; X is CR1R2; R1 is H; R2 is H; Y is NH; and B is isoquinolinyl, substituted with NH₂, and optionally substituted with 1 or 2 further substituents as for heteroaryl^{a}.

Preferably, the compound of formula (I) is a compound of formula (Ia), formula (Ib), formula (Ic), formula (Id), or formula (Ie); X is CR1R2; R1 is H; R2 is H; Y is NH; and B is heteroaryl^{a}. More specifically, the compound of formula (I) is a compound of formula (Ia), formula (Ib), formula (Ic), formula (Id), or formula (Ie); X is CR1R2; R1 is H; R2 is H; Y is NH; and B is selected from: isoquinolinyl, substituted with NH₂ at the 1-position, selected from

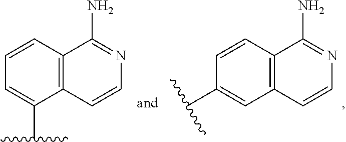 [SMILES: *c1cccc2c(N)nccc12 ; *c1ccc2c(N)nccc2c1]

optionally further substituted with 1 or 2 substituents as for heteroaryl^{a}; 6-azaindolyl

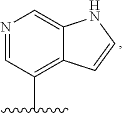 [SMILES: *c1cncc2[nH]ccc12]

optionally substituted as for heteroaryl^{a}; 7-azaindolyl

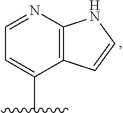 [SMILES: *c1ccnc2[nH]ccc12]

optionally substituted as for heteroaryl^{a}; and pyridyl

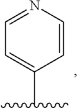 [SMILES: *c1ccncc1]

optionally substituted as for heteroaryl^{a}. More specifically, the compound of formula (I) is a compound of formula (Ia), formula (Ib), formula (Ic), formula (Id), or formula (Ie); X is CR1R2; R1 is H; R2 is H; Y is NH; and B is isoquinolinyl, optionally substituted as for heteroaryl^{a}. More specifically, the compound of formula (I) is a compound of formula (Ia), formula (Ib), formula (Ic), formula (Id), or formula (Ie); X is CR1R2; R1 is H; R2 is H; Y is NH; and B is isoquinolinyl, substituted with NH₂, and optionally substituted with 1 or 2 further substituents as for heteroaryl^{a}.

When B is heteroaryl^{a} and is a 9-membered bicyclic aromatic ring containing a 5-membered ring fused to a 6-membered ring and B is attached to Y via the 6-membered ring, the 9-membered bicyclic aromatic ring preferably contains 1 or 2 ring members independently selected from N, NR12, S and O; and is optionally substituted as for heteroaryl^{a}.

When B is heteroaryl^{a} and is selected from 6-azaindolyl

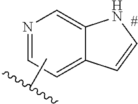

and 7-azaindolyl

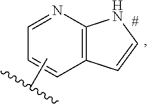

B is preferably optionally substituted as for heteroaryl^{a}, and any optional substituents are, where possible, at any ring member apart from the ring member marked #. It will be understood that the ring member marked # is the ring member shown as “NH”, i.e. the nitrogen as part of the fused, 5-membered, pyrrole ring.

n can be 0, 1 or 2. n can be 0. n can be 1. n can be 2. n can be 1 or 2. Preferably n is 0 or 1. When n is 0, R5 is absent.

When present, (i.e. when n is not 0), R5 can be independently selected from alkyl, cyclopropyl, alkoxy, halo, OH, CN, (CH₂)₀₋₆COOH, and CF₃.

R5 can be independently selected from alkyl, alkoxy, halo, OH, CN, (CH₂)₀₋₆COOH and CF₃.

R5 can be independently selected from CH₃, OH, CH₂OH, OCH₃, OiPr, CF₃, F, Cl, (CH₂)₀₋₆COOH, CN, CH₂F, CHF₂, CH₂OCH₃ and

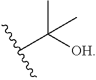 [SMILES: *C(C)(C)O]

R5 can be independently selected from alkyl, alkoxy, halo, CN and CF₃.

R5 can be independently selected from small alkyl, O-(small alkyl), halo, CN and CF₃.

Preferably, R5 is independently selected from CH₃, CH₂OH, OCH₃, OiPr, CF₃, F, CN, and Cl.

Preferably, R5 is independently selected from OCH₃, CF₃, F and Cl.

R5 can be CH₃. R5 can be CH₂OH. R5 can be OCH₃. R5 can be OiPr. R5 can be CF₃. R5 can be F. R5 can be CN. R5 can be Cl.

When Z is a 6-membered ring, R5 is preferably in the ortho or meta substitution with reference to the X substituent.

Preferably, n is 0 or 1; and R5 is independently selected from CH₃, CH₂OH, OCH₃, OiPr, CF₃, F, CN, and Cl.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; n is 0 or 1; and R5 is independently selected from CH₃, CH₂OH, OCH₃, OiPr, CF₃, F, CN, and Cl.

Preferably, Z is selected from pyrazole, phenyl, pyrimidine, pyridine and thiazole; X is CR1R2; R1 is H; R2 is H; Y is NH; n is 0 or 1; and R5 is independently selected from CH₃, CH₂OH, OCH₃, OiPr, CF₃, F, CN, and Cl. More specifically, Z is selected from phenyl, pyrimidine, and pyridine; X is CR1R2; R1 is H; R2 is H; Y is NH; n is 0 or 1; and R5 is independently selected from CH₃, CH₂OH, OCH₃, OiPr, CF₃, F, CN, and Cl.

Preferably, the compound of formula (I) is a compound of formula (Ia), formula (Ib), formula (Ic), formula (Id), or formula (Ie); X is CR1R2; R1 is H; R2 is H; Y is NH; n is 0 or 1; and R5 is independently selected from CH₃, CH₂OH, OCH₃, OiPr, CF₃, F, CN, and Cl.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; B is heteroaryl^{a} and n is 0 or 1; and R5 is independently selected from CH₃, CH₂OH, OCH₃, OiPr, CF₃, F, CN, and Cl.

Preferably, Z is selected from pyrazole, phenyl, pyrimidine, pyridine and thiazole; X is CR1R2; R1 is H; R2 is H; Y is NH; B is heteroaryl^{a} and n is 0 or 1; and R5 is independently selected from CH₃, CH₂OH, OCH₃, OiPr, CF₃, F, CN, and Cl. More specifically, Z is selected from phenyl, pyrimidine, and pyridine; X is CR1R2; R1 is H; R2 is H; Y is NH; and B is heteroaryl^{a}; n is 0 or 1; and R5 is independently selected from CH₃, CH₂OH, OCH₃, OiPr, CF₃, F, CN, and Cl.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is selected from: isoquinolinyl, substituted with NH₂ at the 1-position, selected from

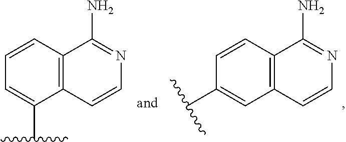 [SMILES: *c1cccc2c(N)nccc12 ; *c1ccc2c(N)nccc2c1]

optionally further substituted with 1 or 2 substituents as for heteroaryl^{a}; 6-azaindolyl

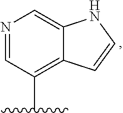 [SMILES: *c1cncc2[nH]ccc12]

optionally substituted as for heteroaryl^{a}; 7-azaindolyl

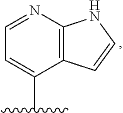 [SMILES: *c1ccnc2[nH]ccc12]

optionally substituted as for heteroaryl^{a}; and pyridyl

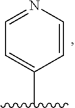 [SMILES: *c1ccncc1]

optionally substituted as for heteroaryl^{a}; n is 0 or 1; and R5 is independently selected from CH₃, CH₂OH, OCH₃, OiPr, CF₃, F, CN, and Cl.

More preferably, Z is selected from pyrazole, phenyl, pyrimidine, pyridine and thiazole; X is CR1R2; R1 is H; R2 is H; Y is NH; B is selected from: isoquinolinyl, substituted with NH₂ at the 1-position, selected from and

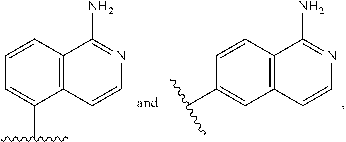 [SMILES: *c1cccc2c(N)nccc12 ; *c1ccc2c(N)nccc2c1]

optionally further substituted with 1 or 2 substituents as for heteroaryl^{a}; 6-azaindolyl

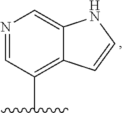 [SMILES: *c1cncc2[nH]ccc12]

optionally substituted as for heteroaryl^{a}; 7-azaindolyl

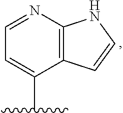 [SMILES: *c1ccnc2[nH]ccc12]

optionally substituted as for heteroaryl^{a}; and pyridyl

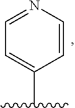 [SMILES: *c1ccncc1]

optionally substituted as for heteroaryl^{a}; n is 0 or 1; and R5 is independently selected from CH₃, CH₂OH, OCH₃, OiPr, CF₃, F, CN, and Cl. More specifically, Z is selected from phenyl, pyrimidine, and pyridine; X is CR1R2; R1 is H; R2 is H; Y is NH; B is selected from: isoquinolinyl, substituted with NH₂ at the 1-position, selected from

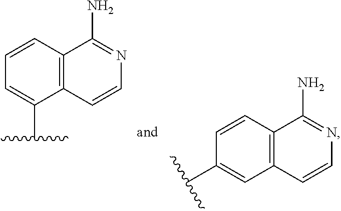 [SMILES: *c1cccc2c(N)nccc12 ; *c1ccc2c(N)nccc2c1]

optionally further substituted with 1 or 2 substituents as for heteroaryl^{a}; 6-azaindolyl

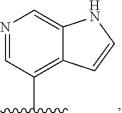 [SMILES: *c1cncc2[nH]ccc12]

optionally substituted as for heteroaryl^{a}; 7-azaindolyl

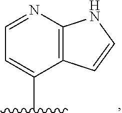 [SMILES: *c1ccnc2[nH]ccc12]

optionally substituted as for heteroaryl^{a}; and pyridyl

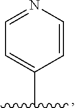 [SMILES: *c1ccncc1]

optionally substituted as for heteroaryl^{a}; n is 0 or 1; and R5 is independently selected from CH₃, CH₂OH, OCH₃, OiPr, CF₃, F, CN, and Cl.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, optionally substituted as for heteroaryl^{a}; n is 0 or 1; and R5 is independently selected from CH₃, CH₂OH, OCH₃, OiPr, CF₃, F, CN, and Cl.

More preferably, Z is selected from pyrazole, phenyl, pyrimidine, pyridine and thiazole; X is CR1R2; R1 is H; R2 is H; Y is NH; B is isoquinolinyl, optionally substituted as for heteroaryl^{a}; n is 0 or 1; and R5 is independently selected from CH₃, CH₂OH, OCH₃, OiPr, CF₃, F, CN, and Cl. More specifically, Z is selected from phenyl, pyrimidine, and pyridine; X is CR1R2; R1 is H; R2 is H; Y is NH; B is isoquinolinyl, optionally substituted as for heteroaryl^{a}; n is 0 or 1; and R5 is independently selected from CH₃, CH₂OH, OCH₃, OiPr, CF₃, F, CN, and Cl.

Yet more preferably Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, substituted with NH₂, and optionally substituted with 1 or 2 further substituents as for heteroaryl^{a}; n is 0 or 1; and R5 is independently selected from CH₃, CH₂OH, OCH₃, OiPr, CF₃, F, CN, and Cl.

Yet more preferably Z is selected from pyrazole, phenyl, pyrimidine, pyridine and thiazole; X is CR1R2; R1 is H; R2 is H; Y is NH; B is isoquinolinyl, substituted with NH₂, and optionally substituted with 1 or 2 further substituents as for heteroaryl^{a}; n is 0 or 1; and R5 is independently selected from CH₃, CH₂OH, OCH₃, OiPr, CF₃, F, CN, and Cl. More specifically, Z is selected from phenyl, pyrimidine, and pyridine; X is CR1R2; R1 is H; R2 is H; Y is NH; B is isoquinolinyl, substituted with NH₂, and optionally substituted with 1 or 2 further substituents as for heteroaryl^{a}; n is 0 or 1; and R5 is independently selected from CH₃, CH₂OH, OCH₃, OiPr, CF₃, F, CN, and Cl.

Preferably, the compound of formula (I) is a compound of formula (Ia), formula (Ib), formula (Ic), formula (Id), or formula (Ie); X is CR1R2; R1 is H; R2 is H; Y is NH; B is heteroaryl^{a}; n is 0 or 1; and R5 is independently selected from CH₃, CH₂OH, OCH₃, OiPr, CF₃, F, CN, and Cl. More specifically, the compound of formula (I) is a compound of formula (Ia), formula (Ib), formula (Ic), formula (Id), or formula (Ie); X is CR1R2; R1 is H; R2 is H; Y is NH; B is selected from: isoquinolinyl, substituted with NH₂ at the 1-position, selected from

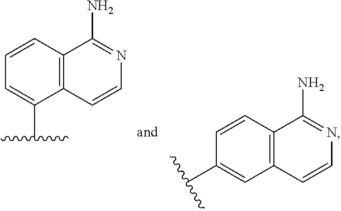 [SMILES: *c1cccc2c(N)nccc12 ; *c1ccc2c(N)nccc2c1]

optionally further substituted with 1 or 2 substituents as for heteroaryl^{a}; 6-azaindolyl

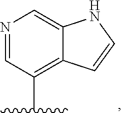 [SMILES: *c1cncc2[nH]ccc12]

optionally substituted as for heteroaryl^{a}; 7-azaindolyl

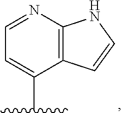 [SMILES: *c1ccnc2[nH]ccc12]

optionally substituted as for heteroaryl^{a}; and pyridyl

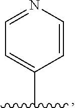 [SMILES: *c1ccncc1]

optionally substituted as for heteroaryl^{a}; n is 0 or 1; and R5 is independently selected from CH₃, CH₂OH, OCH₃, OiPr, CF₃, F, CN, and Cl. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, optionally substituted as for heteroaryl^{a}; n is 0 or 1; and R5 is independently selected from CH₃, CH₂OH, OCH₃, OiPr, CF₃, F, CN, and Cl. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, substituted with NH₂, and optionally substituted with 1 or 2 further substituents as for heteroaryl^{a}; n is 0 or 1; and R5 is independently selected from CH₃, CH₂OH, OCH₃, OiPr, CF₃, F, CN, and Cl.

AW- can be selected from:
—(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—NR12-(CH₂)₁₋₆—C(═O)-A, —(CH₂)₀₋₆—NH—C(═O)—(CH₂)₀₋₆-A, —C(═O)NR12-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A and —SO₂—NH-A.

When A- is —C(═O)NR12-(CH₂)₀₋₆-A, or —(CH₂)₀₋₆—C(═O)—(CH₂)₀₋₆-A, AW- is preferably bonded at a carbon ring member of Z.

AW- can be selected from:
—(CHR12)-A, —O—(CHR12)-A, —(CH₂)₀₋₅-A, —(CH₂)₀₋₅—O—(CH₂)₀₋₅-A, —(CH₂)₀₋₅—NH—(CH₂)₀₋₅-A, —(CH₂)₀₋₅—NR12-(CH₂)₁₋₅—C(═O)-A, —(CH₂)₀₋₅—NH—C(═O)—(CH₂)₀₋₅-A, —C(═O)NR12-(CH₂)₀₋₅-A, —(CH₂)₀₋₅—C(═O)—(CH₂)₀₋₅-A, —(CH₂)₀₋₆-(phenyl)-(CH₂)₀₋₆-A, —NH—SO₂-A and —SO₂—NH-A.

AW- can be selected from:
—(CHR12)-A, —O—(CHR12)-A, —(CH₂)₀₋₄-A, —(CH₂)₀₋₄—O—(CH₂)₀₋₄-A, —(CH₂)₀₋₄—NH—(CH₂)₀₋₄-A, —(CH₂)₀₋₄—NR12-(CH₂)₁₋₄—C(═O)-A, —(CH₂)₀₋₄—NH—C(═O)—(CH₂)₀₋₄-A, —C(═O)NR12-(CH₂)₀₋₄-A, —(CH₂)₀₋₄—C(═O)—(CH₂)₀₋₄-A, —(CH₂)₀₋₄-(phenyl)-(CH₂)₀₋₄-A, —NH—SO₂-A and —SO₂—NH-A.

AW- can be selected from:
—(CHR12)-A, —O—(CHR12)-A, —(CH₂)₀₋₃-A, —(CH₂)₀₋₃—O—(CH₂)₀₋₃-A, —(CH₂)₀₋₃—NH—(CH₂)₀₋₃-A, —(CH₂)₀₋₃—NR12-(CH₂)₁₋₃—C(═O)-A, —(CH₂)₀₋₃—NH—C(═O)—(CH₂)₀₋₃-A, —C(═O)NR12-(CH₂)₀₋₃-A, —(CH₂)₀₋₃—C(═O)—(CH₂)₀₋₃-A, —(CH₂)₀₋₃-(phenyl)-(CH₂)₀₋₃-A, —NH—SO₂-A and —SO₂—NH-A.

Preferably, AW- can be selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A.

More specifically, AW- can be selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A.

More preferably AW- is selected from —O—(CH(CH₃))-A, -A, —OCH₂-A, —CH₂O-A, —C(═O)—(CH₂)-A, —O-A, —(CH₂)₂-A, —NH—CH₂-A and —NH—(CH₂)₂—C(═O)-A.

More specifically AW- is selected from -A, —OCH₂-A, —CH₂O-A, —O-A, —(CH₂)₂-A, —NH—CH₂-A and —NH—(CH₂)₂—C(═O)-A.

A can be a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro; wherein when A is a tricyclic ring system, each of the three rings in the tricyclic ring system is either fused, bridged or spiro to at least one of the other rings in the tricyclic ring system.

A can be a 4- to 15-membered mono-, bi-, or tri-cyclic ring system, containing one N ring member and optionally one, two or three further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro; wherein when A is a tricyclic ring system, each of the three rings in the tricyclic ring system is either fused, bridged or spiro to at least one of the other rings in the tricyclic ring system.

A can be a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

A can be a 4- to 12-membered mono- or bi-cyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. A can be a 4- to 12-membered mono- or bi-cyclic ring system, containing one N ring member and optionally one or two further ring members independently selected from N, optionally wherein the ring system is substituted, where possible, with 1, 2, 3 or 4 substituents independently selected from alkyl and cycloalkyl; wherein when A is a bicyclic ring system, the bicyclic ring system is fused, bridged or spiro. A can be a 4- to 12-membered mono- or bi-cyclic ring system, containing one N ring member and optionally one further ring member independently selected from N, optionally wherein the ring system is substituted, where possible, with 1, 2, 3 or 4 substituents independently selected from alkyl and cycloalkyl; wherein when A is a bicyclic ring system, the bicyclic ring system is fused, bridged or spiro.

A can be a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

A is a 6-membered monocyclic ring system containing one N ring member, wherein the ring system is substituted with 1 substituent selected from alkyl and cycloalkyl. More preferably, A is a 6-membered monocyclic ring system containing one N ring member, wherein the ring system is substituted with 1 alkyl substituent selected from methyl, ethyl, iso-propyl and cyclopropyl. Preferably, the 6-membered monocyclic ring system containing one N ring member is joined to W at the carbon para to the nitrogen.

A can be a 4- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN;
wherein the bicyclic ring system is fused, bridged or spiro.

A can be a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN;
wherein the bicyclic ring system is fused, bridged or spiro.

A can be a fused 6- to 12-membered bicyclic ring system containing one N ring member and optionally one, two or three further ring members independently selected from N, O and S, wherein the fused ring system consists of an aromatic ring fused to a non-aromatic ring, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN. A can be a fused 6- to 12-membered bicyclic ring system containing one N ring member and optionally one, two or three further ring members independently selected from N, O and S, wherein the fused ring system consists of an aromatic ring fused to a non-aromatic ring, optionally wherein the ring system is substituted, where possible, with 1, 2, 3 or 4 substituents independently selected from alkyl and CF₃.

A can be a fused 6- to 12-membered bicyclic ring system containing one N ring member and optionally one, two or three further ring members independently selected from N, O and S, wherein the fused ring system consists of a 5-membered aromatic ring fused to a 6-membered non-aromatic ring, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN. A can be a fused 6- to 12-membered bicyclic ring system containing one N ring member and optionally one, two or three further ring members independently selected from N, O and S, wherein the fused ring system consists of a 5-membered aromatic ring fused to a 6-membered non-aromatic ring, optionally wherein the ring system is substituted, where possible, with 1, 2, 3 or 4 substituents independently selected from alkyl and CF₃.

A can be selected from:

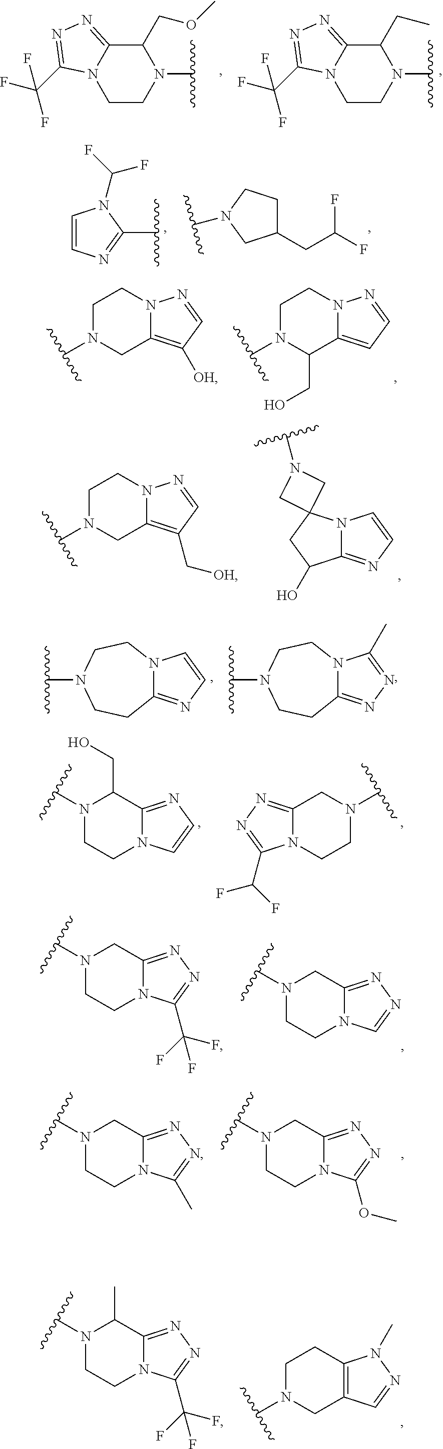

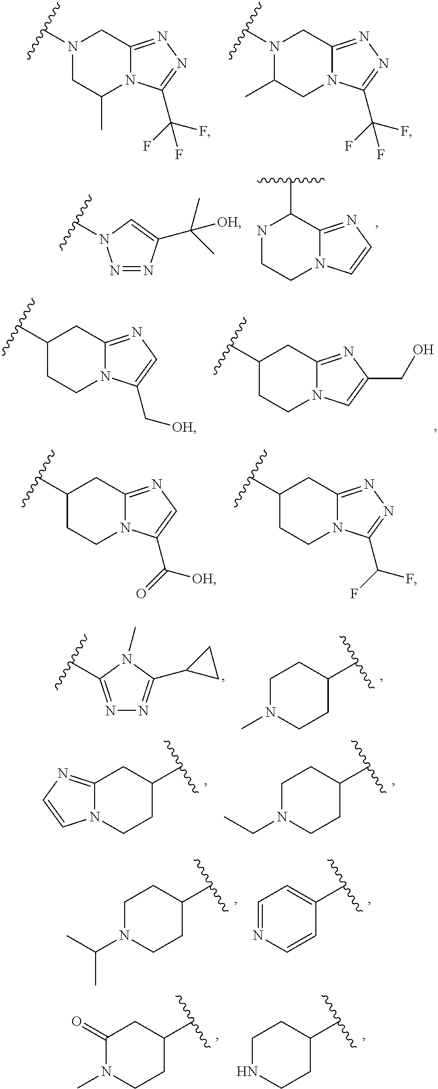

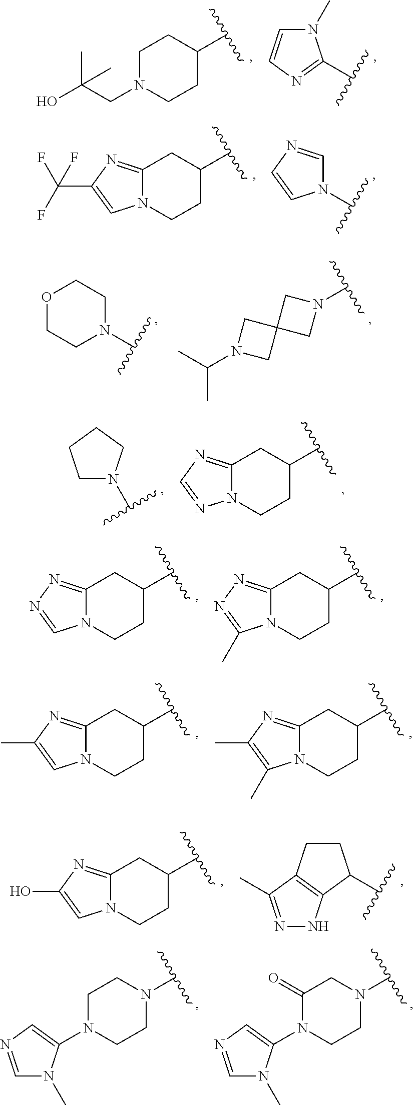

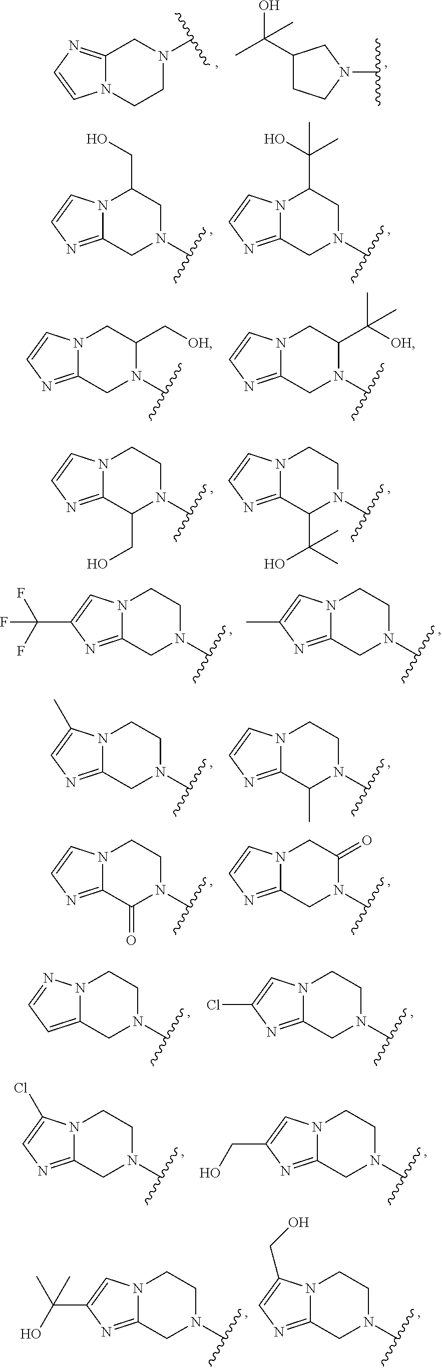

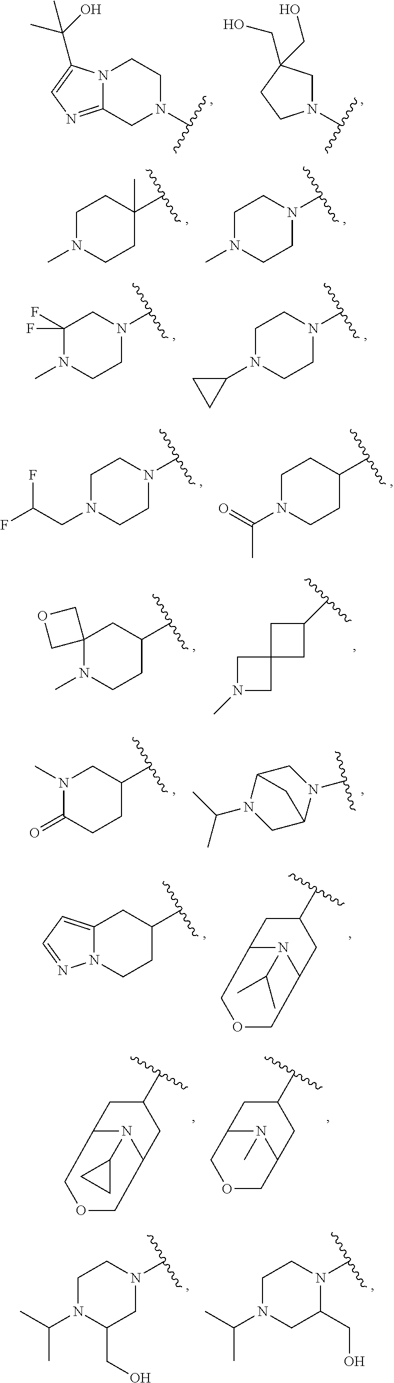

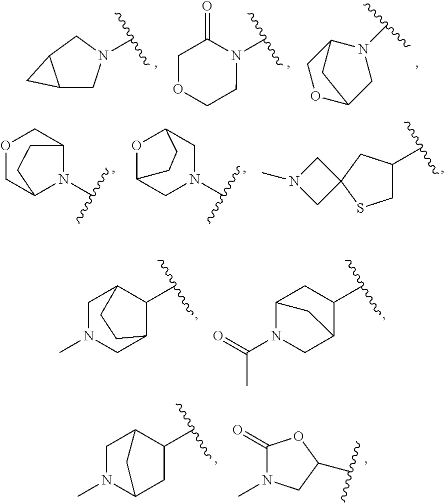

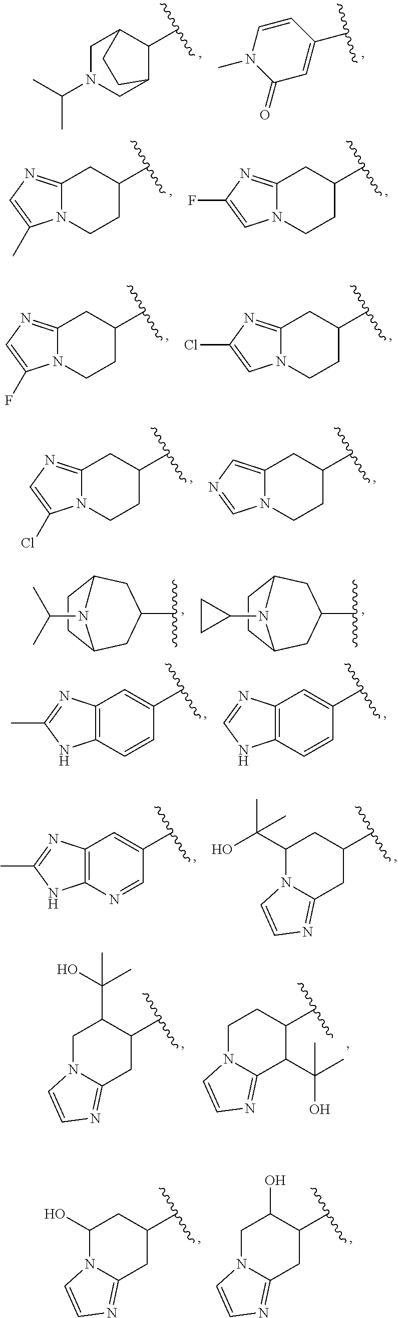

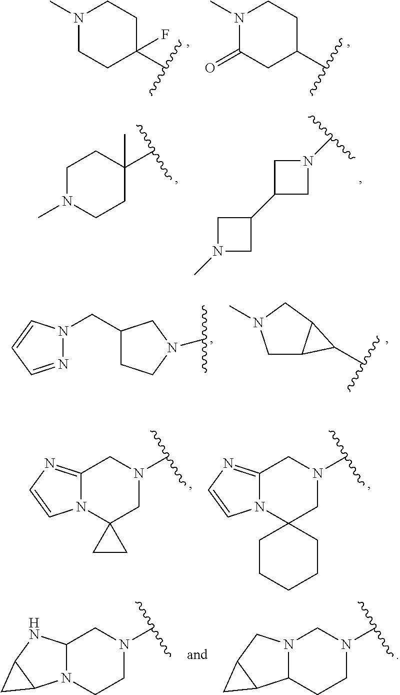

A can be selected from:

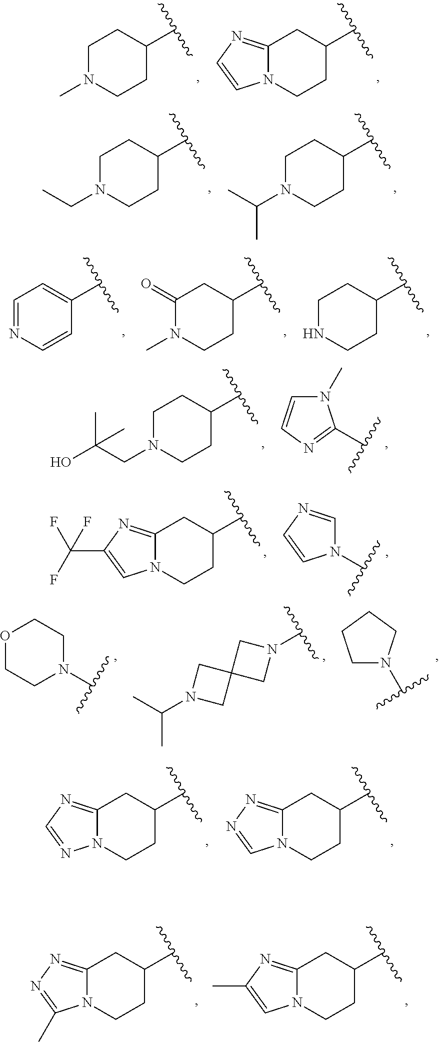

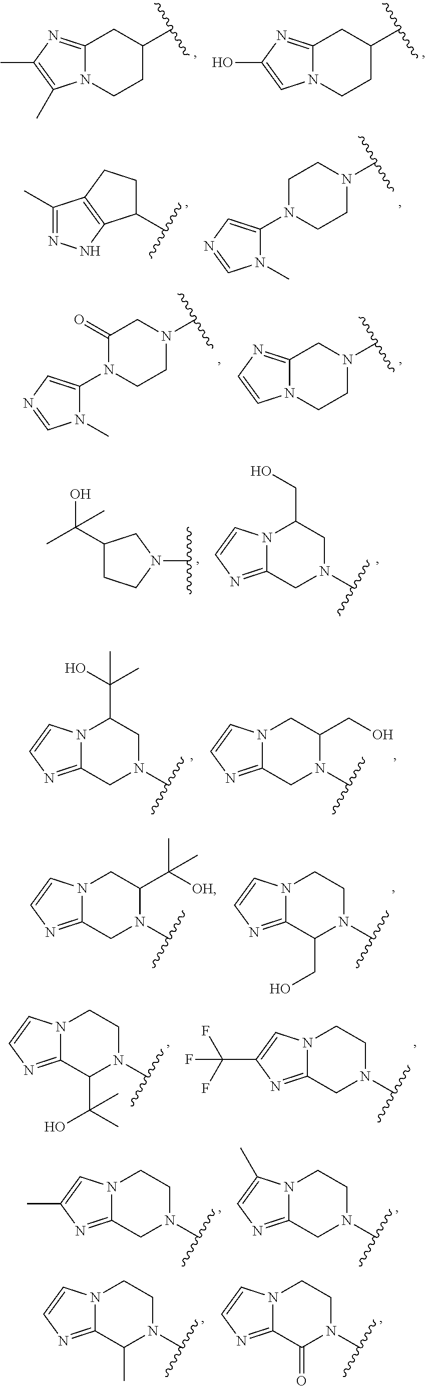

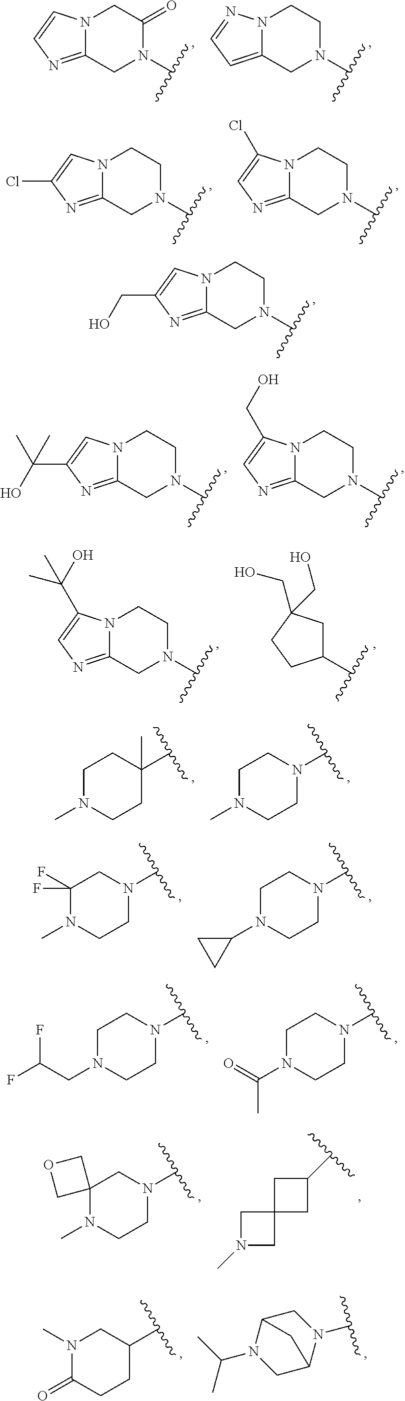

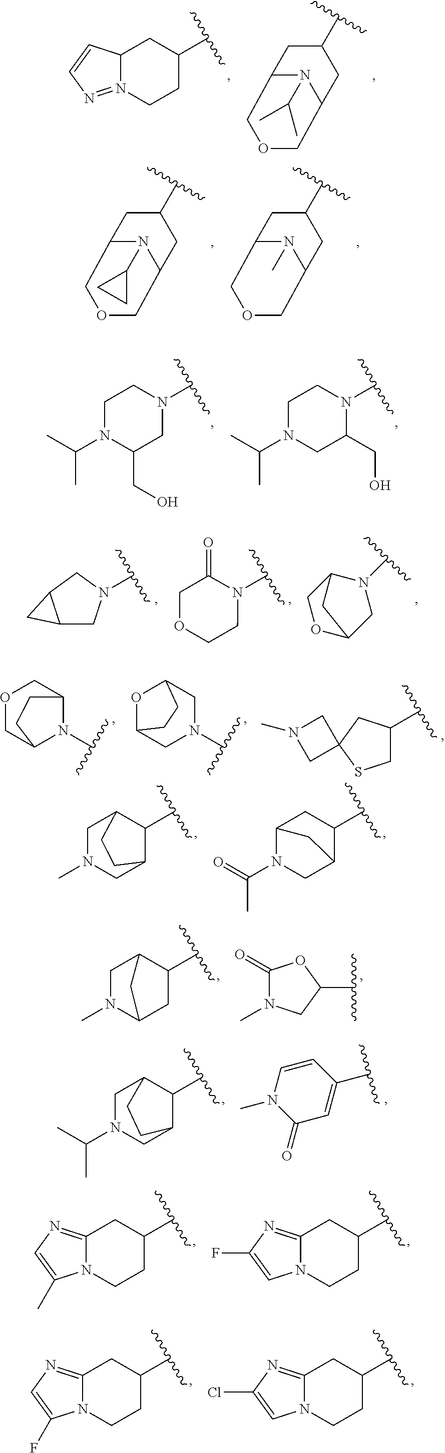

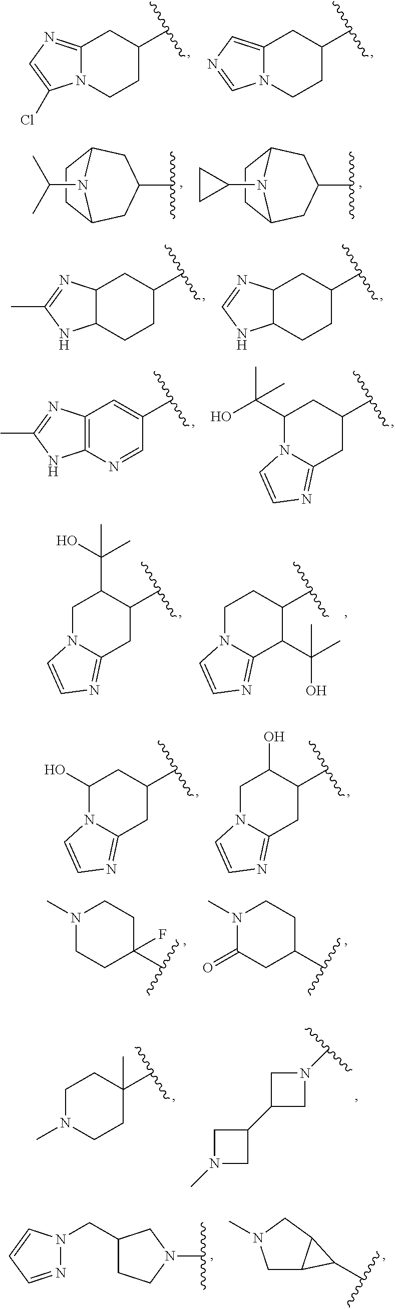

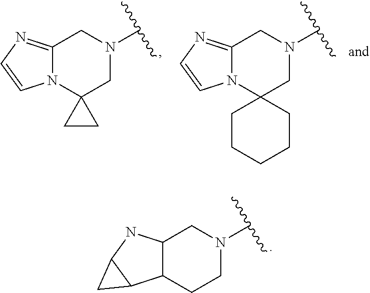 [SMILES: *N1Cc2nccn2C2(CC2)C1 ; *N1Cc2nccn2C2(CCCCC2)C1 ; *N1CCC2C(C1)NC1CC12]

A can be selected from:

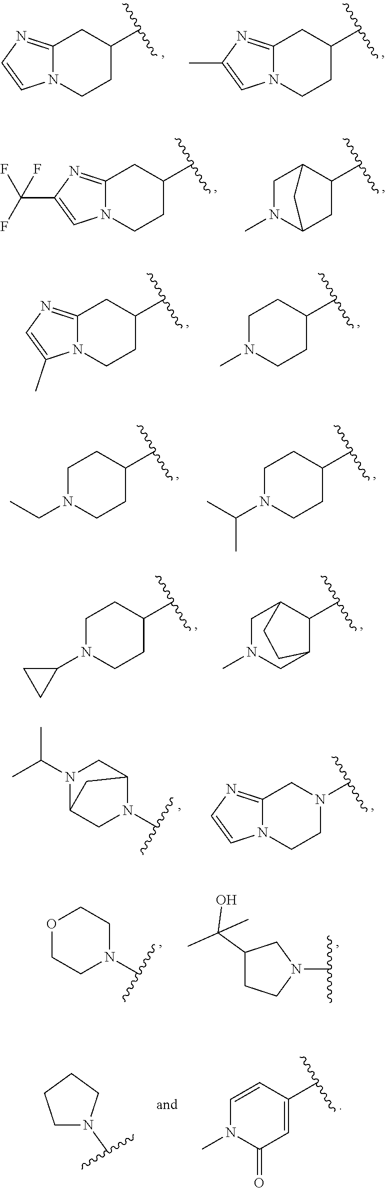

A can be selected from:

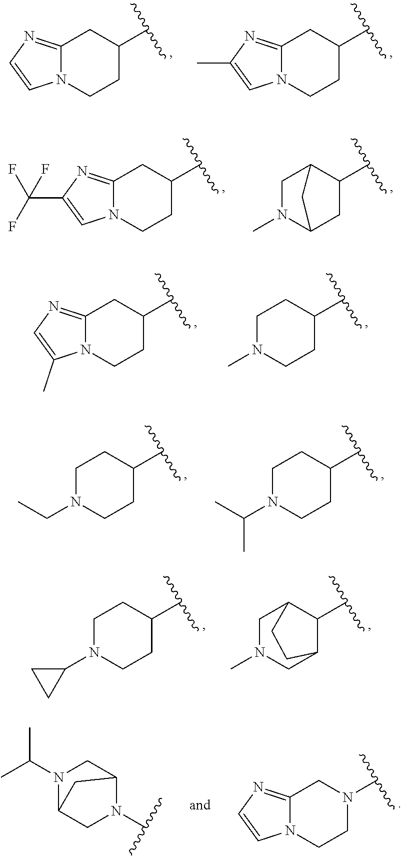

Preferably, A is selected from:

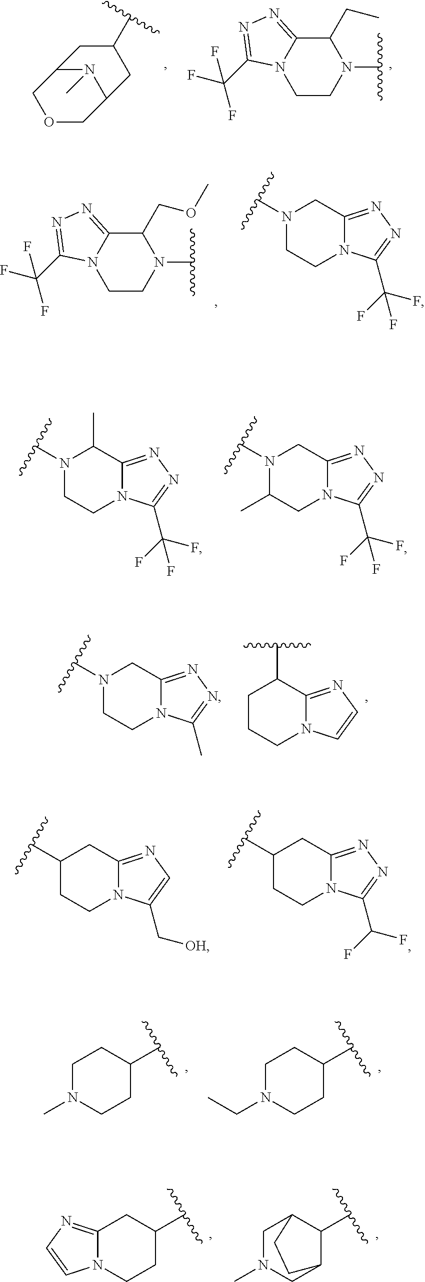

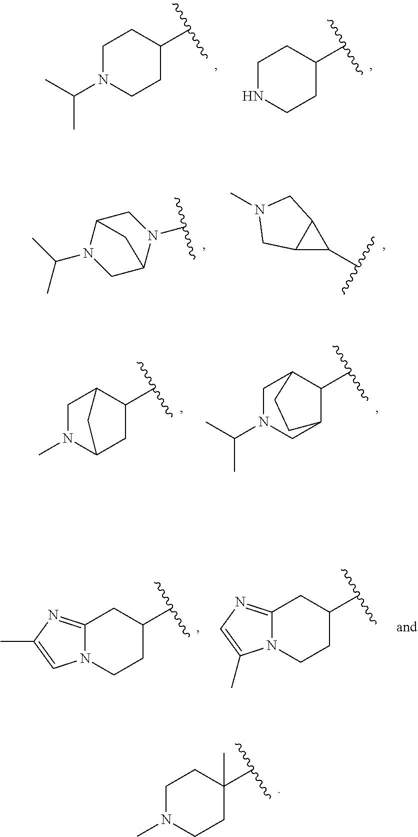

Preferably, A is selected from:

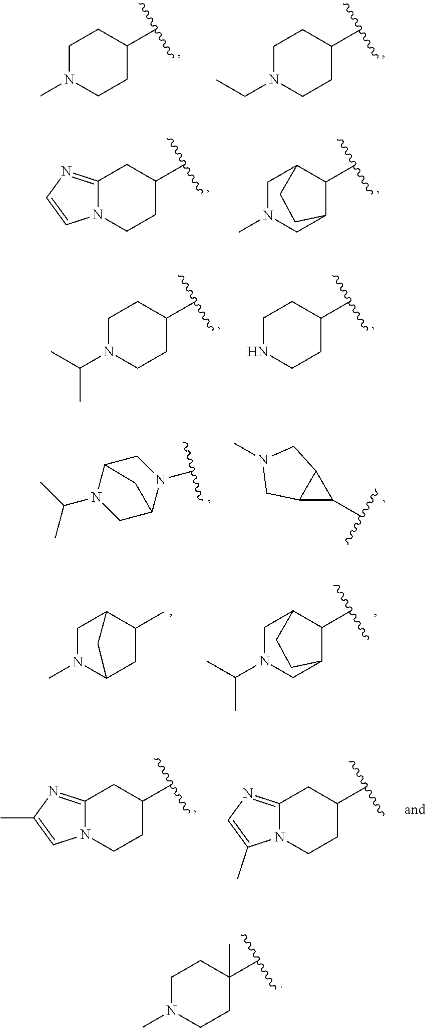

More preferably, A is selected from:

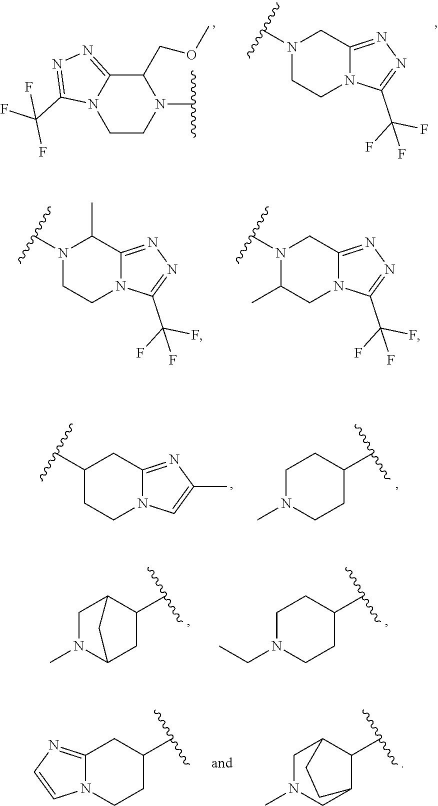

More preferably, A is selected from:

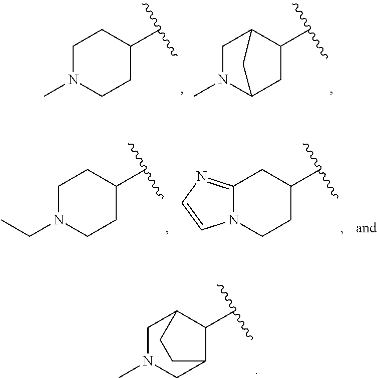

Preferably AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine and thiazole; X is CR1R2; R1 is H; R2 is H; Y is NH; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine and thiazole; X is CR1R2; R1 is H; R2 is H; Y is NH; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, the compound of formula (I) is a compound of formula (Ia), formula (Ib), formula (Ic), formula (Id), or formula (Ie); X is CR1R2; R1 is H; R2 is H; Y is NH; AW- is selected from: —O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, the compound of formula (I) is a compound of formula (Ia), formula (Ib), formula (Ic), formula (Id), or formula (Ie); X is CR1R2; R1 is H; R2 is H; Y is NH; AW- is selected from: —O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; B is heteroaryl^{a}; AW- is selected from: —O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine and thiazole; X is CR1R2; R1 is H; R2 is H; Y is NH; B is heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; B is heteroaryl^{a}; AW- is selected from: —O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine and thiazole; X is CR1R2; R1 is H; R2 is H; Y is NH; B is heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is selected from: isoquinolinyl, substituted with NH₂ at the 1-position, selected from

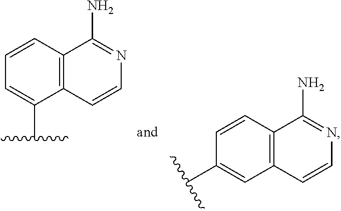 [SMILES: *c1cccc2c(N)nccc12 ; *c1ccc2c(N)nccc2c1]

optionally further substituted with 1 or 2 substituents as for heteroaryl^{a}; 6-azaindolyl

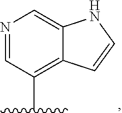 [SMILES: *c1cncc2[nH]ccc12]

optionally substituted as for heteroaryl^{a}; 7-azaindolyl

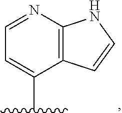 [SMILES: *c1ccnc2[nH]ccc12]

optionally substituted as for heteroaryl^{a}; and pyridyl

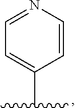 [SMILES: *c1ccncc1]

optionally substituted as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine and thiazole; X is CR1R2; R1 is H; R2 is H; Y is NH; B is selected from: isoquinolinyl, substituted with NH₂ at the 1-position, selected from

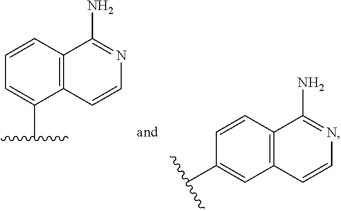 [SMILES: *c1cccc2c(N)nccc12 ; *c1ccc2c(N)nccc2c1]

optionally further substituted with 1 or 2 substituents as for heteroaryl^{a}; 6-azaindolyl

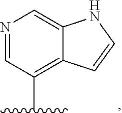 [SMILES: *c1cncc2[nH]ccc12]

optionally substituted as for heteroaryl^{a}; 7-azaindolyl

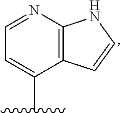 [SMILES: *c1ccnc2[nH]ccc12]

optionally substituted as for heteroaryl^{a}; and pyridyl

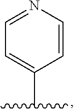 [SMILES: *c1ccncc1]

optionally substituted as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is selected from: isoquinolinyl, substituted with NH₂ at the 1-position, selected from

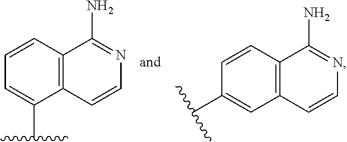 [SMILES: *c1cccc2c(N)nccc12 ; *c1ccc2c(N)nccc2c1]

optionally further substituted with 1 or 2 substituents as for heteroaryl^{a}; 6-azaindolyl

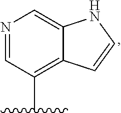 [SMILES: *c1cncc2[nH]ccc12]

optionally substituted as for heteroaryl^{a}; 7-azaindolyl,

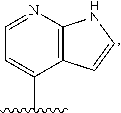 [SMILES: *c1ccnc2[nH]ccc12]

optionally substituted as for heteroaryl^{a}; and pyridyl

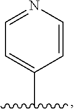 [SMILES: *c1ccncc1]

optionally substituted as for heteroaryl^{a}, optionally substituted as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, optionally substituted as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine and thiazole; X is CR1R2; R1 is H; R2 is H; Y is NH; B is isoquinolinyl, optionally substituted as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is isoquinolinyl, optionally substituted as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is selected from: isoquinolinyl, substituted with NH₂ at the 1-position, selected from

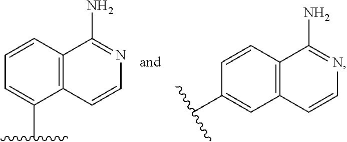 [SMILES: *c1cccc2c(N)nccc12 ; *c1ccc2c(N)nccc2c1]

optionally further substituted with 1 or 2 substituents as for heteroaryl^{a}; 6-azaindolyl

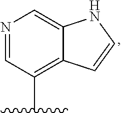 [SMILES: *c1cncc2[nH]ccc12]

optionally substituted as for heteroaryl^{a}; 7-azaindolyl

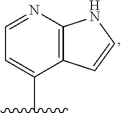 [SMILES: *c1ccnc2[nH]ccc12]

optionally substituted as for heteroaryl^{a}; and pyridyl

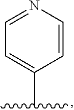 [SMILES: *c1ccncc1]

optionally substituted as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine and thiazole; X is CR1R2; R1 is H; R2 is H; Y is NH; B is selected from: isoquinolinyl, substituted with NH₂ at the 1-position, selected from

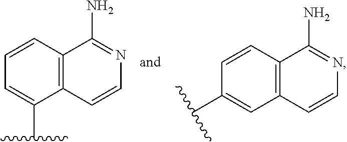 [SMILES: *c1cccc2c(N)nccc12 ; *c1ccc2c(N)nccc2c1]

optionally further substituted with 1 or 2 substituents as for heteroaryl^{a}; 6-azaindolyl

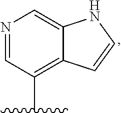 [SMILES: *c1cncc2[nH]ccc12]

optionally substituted as for heteroaryl^{a}; 7-azaindolyl

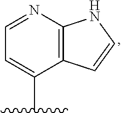 [SMILES: *c1ccnc2[nH]ccc12]

optionally substituted as for heteroaryl^{a}; and pyridyl

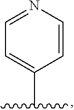 [SMILES: *c1ccncc1]

optionally substituted as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is selected from: isoquinolinyl, substituted with NH₂ at the 1-position, selected from

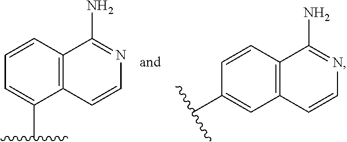 [SMILES: *c1cccc2c(N)nccc12 ; *c1ccc2c(N)nccc2c1]

optionally further substituted with 1 or 2 substituents as for heteroaryl^{a}; 6-azaindolyl

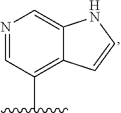 [SMILES: *c1cncc2[nH]ccc12]

optionally substituted as for heteroaryl^{a}; 7-azaindolyl

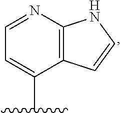 [SMILES: *c1ccnc2[nH]ccc12]

optionally substituted as for heteroaryl^{a}; and pyridyl

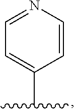 [SMILES: *c1ccncc1]

optionally substituted as for heteroaryl^{a}; AW- is selected from: —O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, optionally substituted as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine and thiazole; X is CR1R2; R1 is H; R2 is H; Y is NH; B is isoquinolinyl, optionally substituted as for heteroaryl^{a}; AW- is selected from: —O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is isoquinolinyl, optionally substituted as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Yet more preferably Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, substituted with NH₂, and optionally substituted with 1 or 2 further substituents as for heteroaryl^{a}; AW- is selected from: —O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Yet more preferably Z is selected from pyrazole, phenyl, pyrimidine, pyridine and thiazole; X is CR1R2; R1 is H; R2 is H; Y is NH; B is isoquinolinyl, substituted with NH₂, and optionally substituted with 1 or 2 further substituents as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is isoquinolinyl, substituted with NH₂, and optionally substituted with 1 or 2 further substituents as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Yet more preferably Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, substituted with NH₂, and optionally substituted with 1 or 2 further substituents as for heteroaryl^{a}; AW- is selected from: —O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Yet more preferably Z is selected from pyrazole, phenyl, pyrimidine, pyridine and thiazole; X is CR1R2; R1 is H; R2 is H; Y is NH; B is isoquinolinyl, substituted with NH₂, and optionally substituted with 1 or 2 further substituents as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is isoquinolinyl, substituted with NH₂, and optionally substituted with 1 or 2 further substituents as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, the compound of formula (I) is a compound of formula (Ia), formula (Ib), formula (Ic), formula (Id), or formula (Ie); X is CR1R2; R1 is H; R2 is H; Y is NH; B is heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is selected from: isoquinolinyl, substituted with NH₂ at the 1-position, selected from

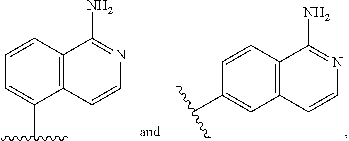 [SMILES: *c1cccc2c(N)nccc12 ; *c1ccc2c(N)nccc2c1]

optionally further substituted with 1 or 2 substituents as for heteroaryl^{a}; 6-azaindolyl

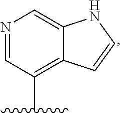 [SMILES: *c1cncc2[nH]ccc12]

optionally substituted as for heteroaryl^{a}; 7-azaindolyl

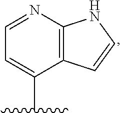 [SMILES: *c1ccnc2[nH]ccc12]

optionally substituted as for heteroaryl^{a}; and pyridyl

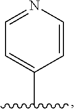 [SMILES: *c1ccncc1]

optionally substituted as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, optionally substituted as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, substituted with NH₂, and optionally substituted with 1 or 2 further substituents as for heteroaryl^{a}; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, the compound of formula (I) is a compound of formula (Ia), formula (Ib), formula (Ic), formula (Id), or formula (Ie); X is CR1R2; R1 is H; R2 is H; Y is NH; B is heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is selected from: isoquinolinyl, substituted with NH₂ at the 1-position, selected from

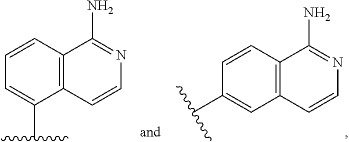 [SMILES: *c1cccc2c(N)nccc12 ; *c1ccc2c(N)nccc2c1]

optionally further substituted with 1 or 2 substituents as for heteroaryl^{a}; 6-azaindolyl

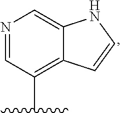 [SMILES: *c1cncc2[nH]ccc12]

optionally substituted as for heteroaryl^{a}; 7-azaindolyl

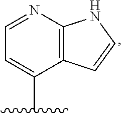 [SMILES: *c1ccnc2[nH]ccc12]

optionally substituted as for heteroaryl^{a}; and pyridyl

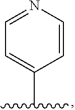 [SMILES: *c1ccncc1]

optionally substituted as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, optionally substituted as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, substituted with NH₂, and optionally substituted with 1 or 2 further substituents as for heteroaryl^{a}; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine and thiazole; X is CR1R2; R1 is H; R2 is H; Y is NH; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine and thiazole; X is CR1R2; R1 is H; R2 is H; Y is NH; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, the compound of formula (I) is a compound of formula (Ia), formula (Ib), formula (Ic), formula (Id), or formula (Ie); X is CR1R2; R1 is H; R2 is H; Y is NH; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, the compound of formula (I) is a compound of formula (Ia), formula (Ib), formula (Ic), formula (Id), or formula (Ie); X is CR1R2; R1 is H; R2 is H; Y is NH; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; B is heteroaryl^{a} and n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine and thiazole; X is CR1R2; R1 is H; R2 is H; Y is NH; B is heteroaryl^{a} and n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; and B is heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine, pyrazine, pyridazine, oxazole, thiophene, and thiazole; X is CR1R2; R1 is H; R2 is H; Y is NH; B is heteroaryl^{a} and n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, Z is selected from pyrazole, phenyl, pyrimidine, pyridine and thiazole; X is CR1R2; R1 is H; R2 is H; Y is NH; B is heteroaryl^{a} and n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is heteroaryl^{a}; n is 0 or 1; and R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is selected from: isoquinolinyl, substituted with NH₂ at the 1-position, selected from

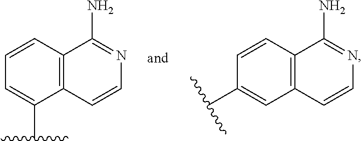 [SMILES: *c1cccc2c(N)nccc12 ; *c1ccc2c(N)nccc2c1]

optionally further substituted with 1 or 2 substituents as for heteroaryl^{a}; 6-azaindolyl

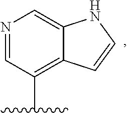 [SMILES: *c1cncc2[nH]ccc12]

optionally substituted as for heteroaryl^{a}; 7-azaindolyl

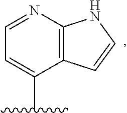 [SMILES: *c1ccnc2[nH]ccc12]

optionally substituted as for heteroaryl^{a}; and pyridyl

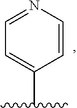 [SMILES: *c1ccncc1]

optionally substituted as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine and thiazole; X is CR1R2; R1 is H; R2 is H; Y is NH; B is selected from: isoquinolinyl, substituted with NH₂ at the 1-position, selected from

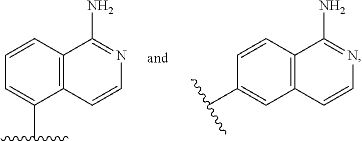 [SMILES: *c1cccc2c(N)nccc12 ; *c1ccc2c(N)nccc2c1]

optionally further substituted with 1 or 2 substituents as for heteroaryl^{a}; 6-azaindolyl

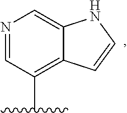 [SMILES: *c1cncc2[nH]ccc12]

optionally substituted as for heteroaryl^{a}; 7-azaindolyl

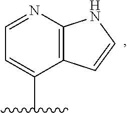 [SMILES: *c1ccnc2[nH]ccc12]

optionally substituted as for heteroaryl^{a}; and pyridyl

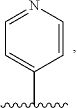 [SMILES: *c1ccncc1]

optionally substituted as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is selected from: isoquinolinyl, substituted with NH₂ at the 1-position, selected from

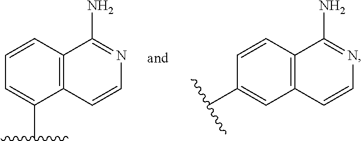 [SMILES: *c1cccc2c(N)nccc12 ; *c1ccc2c(N)nccc2c1]

optionally further substituted with 1 or 2 substituents as for heteroaryl^{a}; 6-azaindolyl

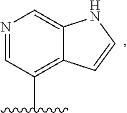 [SMILES: *c1cncc2[nH]ccc12]

optionally substituted as for heteroaryl^{a}; 7-azaindolyl

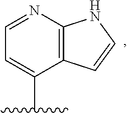 [SMILES: *c1ccnc2[nH]ccc12]

optionally substituted as for heteroaryl^{a}; and pyridyl

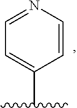 [SMILES: *c1ccncc1]

optionally substituted as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is selected from: isoquinolinyl, substituted with NH₂ at the 1-position, selected from

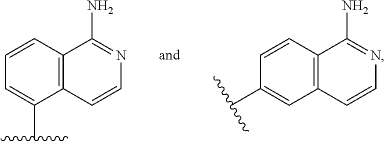 [SMILES: *c1cccc2c(N)nccc12 ; *c1ccc2c(N)nccc2c1]

optionally further substituted with 1 or 2 substituents as for heteroaryl^{a}; 6-azaindolyl

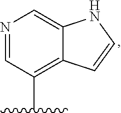 [SMILES: *c1cncc2[nH]ccc12]

optionally substituted as for heteroaryl^{a}; 7-azaindolyl

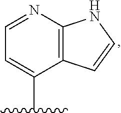 [SMILES: *c1ccnc2[nH]ccc12]

optionally substituted as for heteroaryl^{a}; and pyridyl

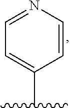 [SMILES: *c1ccncc1]

optionally substituted as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine and thiazole; X is CR1R2; R1 is H; R2 is H; Y is NH; B is selected from: isoquinolinyl, substituted with NH₂ at the 1-position, selected from

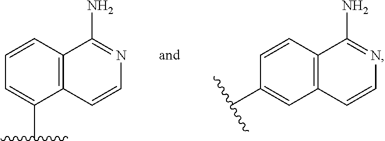 [SMILES: *c1cccc2c(N)nccc12 ; *c1ccc2c(N)nccc2c1]

optionally further substituted with 1 or 2 substituents as for heteroaryl^{a}; 6-azaindolyl

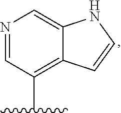 [SMILES: *c1cncc2[nH]ccc12]

optionally substituted as for heteroaryl^{a}; 7-azaindolyl

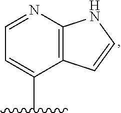 [SMILES: *c1ccnc2[nH]ccc12]

optionally substituted as for heteroaryl^{a}; and pyridyl

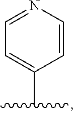 [SMILES: *c1ccncc1]

optionally substituted as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is selected from: isoquinolinyl, substituted with NH₂ at the 1-position, selected from

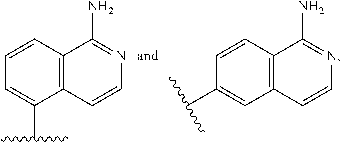 [SMILES: *c1cccc2c(N)nccc12 ; "and" ; *c1ccc2c(N)nccc2c1]

optionally further substituted with 1 or 2 substituents as for heteroaryl^{a}; 6-azaindolyl

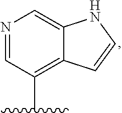 [SMILES: *c1cncc2[nH]ccc12]

optionally substituted as for heteroaryl^{a}; 7-azaindolyl

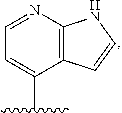 [SMILES: *c1ccnc2[nH]ccc12]

optionally substituted as for heteroaryl^{a}; and pyridyl

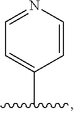 [SMILES: *c1ccncc1]

optionally substituted as for heteroaryl^{a}; n is 0 or 1; R5 is independently selected from CH₃, CH₂OH, OCH₃, OiPr, CF₃, F, CN, and Cl, AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, optionally substituted as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine and thiazole; X is CR1R2; R1 is H; R2 is H; Y is NH; B is isoquinolinyl, optionally substituted as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is isoquinolinyl, optionally substituted as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, optionally substituted as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

More preferably, Z is selected from pyrazole, phenyl, pyrimidine, pyridine and thiazole; X is CR1R2; R1 is H; R2 is H; Y is NH; B is isoquinolinyl, optionally substituted as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is isoquinolinyl, optionally substituted as for heteroaryl^{a}; n is 0 or 1; R5 is independently selected from CH₃, CH₂OH, OCH₃, OiPr, CF₃, F, CN, and Cl, AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Yet more preferably Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, substituted with NH₂, and optionally substituted with 1 or 2 further substituents as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Yet more preferably Z is selected from pyrazole, phenyl, pyrimidine, pyridine and thiazole; X is CR1R2;
R1 is H; R2 is H; Y is NH; B is isoquinolinyl, substituted with NH₂, and optionally substituted with 1 or 2 further substituents as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is isoquinolinyl, substituted with NH₂, and optionally substituted with 1 or 2 further substituents as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Yet more preferably Z is selected from pyrazole, phenyl, pyrimidine, pyridine, pyrazine, pyridazine, oxazole, thiophene, and thiazole; X is CR1R2; R1 is H; R2 is H; Y is NH; B is isoquinolinyl, substituted with NH₂, and optionally substituted with 1 or 2 further substituents as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Yet more preferably Z is selected from pyrazole, phenyl, pyrimidine, pyridine and thiazole; X is CR1R2; R1 is H; R2 is H; Y is NH; B is isoquinolinyl, substituted with NH₂, and optionally substituted with 1 or 2 further substituents as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, Z is selected from phenyl, pyrimidine, and pyridine; X is CR1R2; R1 is H; R2 is H; Y is NH; B is isoquinolinyl, substituted with NH₂, and optionally substituted with 1 or 2 further substituents as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, the compound of formula (I) is a compound of formula (Ia), formula (Ib), formula (Ic), formula (Id), or formula (Ie); X is CR1R2; R1 is H; R2 is H; Y is NH; B is heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is selected from: isoquinolinyl, substituted with NH₂ at the 1-position, selected from

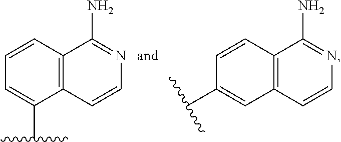 [SMILES: *c1cccc2c(N)nccc12 ; "and" ; *c1ccc2c(N)nccc2c1]

optionally further substituted with 1 or 2 substituents as for heteroaryl; 6-azaindolyl

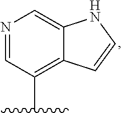 [SMILES: *c1cncc2[nH]ccc12]

optionally substituted as for heteroaryl; 7-azaindolyl

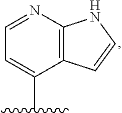 [SMILES: *c1ccnc2[nH]ccc12]

optionally substituted as for heteroaryl^{a} and pyridyl

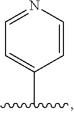 [SMILES: *c1ccncc1]

optionally substituted as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, optionally substituted as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, substituted with NH₂, and optionally substituted with 1 or 2 further substituents as for heteroaryl^{a}; n is 0 or 1; R5 is independently selected from CH₃, CH₂OH, OCH₃, OiPr, CF₃, F, CN, and Cl; AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

Preferably, the compound of formula (I) is a compound of formula (Ia), formula (Ib), formula (Ic), formula (Id), or formula (Ie); X is CR1R2; R1 is H; R2 is H; Y is NH; B is heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is selected from: isoquinolinyl, substituted with NH₂ at the 1-position, selected from

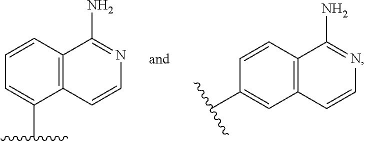 [SMILES: *c1cccc2c(N)nccc12 ; *c1ccc2c(N)nccc2c1]

optionally further substituted with 1 or 2 substituents as for heteroaryl^{a}; 6-azaindolyl

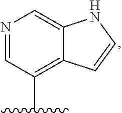 [SMILES: *c1cncc2[nH]ccc12]

optionally substituted as for heteroaryl^{a}; 7-azaindolyl,

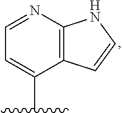 [SMILES: *c1ccnc2[nH]ccc12]

optionally substituted as for heteroaryl^{a}; and pyridyl,

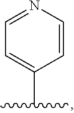 [SMILES: *c1ccncc1]

optionally substituted as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, optionally substituted as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro. More specifically, the compound of formula (I) is a compound of formula (Ia), formula (Ib), formula (Ic), formula (Id), or formula (Ie); X is CR1R2; R1 is H; R2 is H; Y is NH; B is isoquinolinyl, substituted with NH₂, and optionally substituted with 1 or 2 further substituents as for heteroaryl^{a}; n is 0 or 1; R5 is independently selected from CH₃, CH₂OH, OCH₃, OiPr, CF₃, F, CN, and Cl; AW- is selected from:
—O—(CH(CH₃))-A, -A, —OCH₂-A, —CH₂O-A, —C(═O)—(CH₂)-A-O-A, —(CH₂)₂-A, —NH—CH₂-A and —NH—(CH₂)₂—C(═O)-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

For the compounds provided in Tables 1a, 1b, 2a, 2b, 3, 4a, 4b, 5a, 5b, 6, 7, 8a, 8b, 8c, 9, and 10 below, where stereochemistry is indicated, the compound is intended to cover all possible stereoisomers thereof.

The present invention therefore provides the compounds below in Tables 1a, 1b, 2a, 2b, 3, 4a, 4b, 5a, 5b, 6, 7, 8a, 8b, 8c, 9, and 10, and pharmaceutically acceptable salts and/or solvates thereof. The present invention therefore also provides stereoisomers of the compounds below in Tables 1a, 1b, 2a, 2b, 3, 4a, 4b, 5a, 5b, 6, 7, 8a, 8b, 8c, 9, and 10, and pharmaceutically acceptable salts and/or solvates thereof.

The present invention therefore provides the compounds below in Tables 1a, 2a, 3, 4a, 5a, 6, 7, and 8a, and pharmaceutically acceptable salts and/or solvates thereof. The present invention therefore also provides stereoisomers of the compounds below in Tables 1a, 2a, 3, 4a, 5a, 6, 7, and 8a, and pharmaceutically acceptable salts and/or solvates thereof.

The present invention provides compounds selected from Table 1a, and pharmaceutically acceptable salts and/or solvates thereof. The present invention also provides stereoisomers of the compounds selected from Table 1a, and pharmaceutically acceptable salts and/or solvates thereof.

The present invention provides compounds selected from Table 1b, and pharmaceutically acceptable salts and/or solvates thereof. The present invention also provides stereoisomers of the compounds selected from Table 1b, and pharmaceutically acceptable salts and/or solvates thereof.

The present invention provides compounds selected from Table 2a, and pharmaceutically acceptable salts and/or solvates thereof. The present invention also provides stereoisomers of the compounds selected from Table 2a, and pharmaceutically acceptable salts and/or solvates thereof.

The present invention provides compounds selected from Table 2b, and pharmaceutically acceptable salts and/or solvates thereof. The present invention also provides stereoisomers of the compounds selected from Table 2b, and pharmaceutically acceptable salts and/or solvates thereof.

The present invention provides compounds selected from Table 3, and pharmaceutically acceptable salts and/or solvates thereof. The present invention also provides stereoisomers of the compounds selected from Table 3, and pharmaceutically acceptable salts and/or solvates thereof.

The present invention provides compounds selected from Table 4a, and pharmaceutically acceptable salts and/or solvates thereof. The present invention also provides stereoisomers of the compounds selected from Table 4a, and pharmaceutically acceptable salts and/or solvates thereof.

The present invention provides compounds selected from Table 4b, and pharmaceutically acceptable salts and/or solvates thereof. The present invention also provides stereoisomers of the compounds selected from Table 4b, and pharmaceutically acceptable salts and/or solvates thereof.

The present invention provides compounds selected from Table 5a, and pharmaceutically acceptable salts and/or solvates thereof. The present invention also provides stereoisomers of the compounds selected from Table 5a, and pharmaceutically acceptable salts and/or solvates thereof.

The present invention provides compounds selected from Table 5b, and pharmaceutically acceptable salts and/or solvates thereof. The present invention also provides stereoisomers of the compounds selected from Table 5b, and pharmaceutically acceptable salts and/or solvates thereof.

The present invention provides compounds selected from Table 6, and pharmaceutically acceptable salts and/or solvates thereof. The present invention also provides stereoisomers of the compounds selected from Table 6, and pharmaceutically acceptable salts and/or solvates thereof.

The present invention provides compounds selected from Table 7, and pharmaceutically acceptable salts and/or solvates thereof. The present invention also provides stereoisomers of the compounds selected from Table 7, and pharmaceutically acceptable salts and/or solvates thereof.

The present invention provides compounds selected from Table 8a, and pharmaceutically acceptable salts and/or solvates thereof. The present invention also provides stereoisomers of the compounds selected from Table 8a, and pharmaceutically acceptable salts and/or solvates thereof.

The present invention provides compounds selected from Table 8b, and pharmaceutically acceptable salts and/or solvates thereof. The present invention also provides stereoisomers of the compounds selected from Table 8b, and pharmaceutically acceptable salts and/or solvates thereof.

The present invention provides compounds selected from Table 8c, and pharmaceutically acceptable salts and/or solvates thereof. The present invention also provides stereoisomers of the compounds selected from Table 8c, and pharmaceutically acceptable salts and/or solvates thereof.

The present invention provides compounds selected from Table 9, and pharmaceutically acceptable salts and/or solvates thereof. The present invention also provides stereoisomers of the compounds selected from Table 9, and pharmaceutically acceptable salts and/or solvates thereof.

The present invention provides compounds selected from Table 10, and pharmaceutically acceptable salts and/or solvates thereof. The present invention also provides stereoisomers of the compounds selected from Table 10, and pharmaceutically acceptable salts and/or solvates thereof.

It will be understood that, when reading the compounds in Tables 1a, 1b, 2a, 2b, 3, 4a, 4b, 5a, 5b, 6, 7, 8a, 8b, 8c, 9, and 10 below, the substituents are to be read from left to right. For example, example compound 2185 in Table 2a has a Q₁ group:

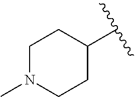 [SMILES: *C1CCN(C)CC1]

and a Q₂ group “OCH₂”. Therefore, the Q₁ group is attached to the “O” of the “OCH₂” of the Q₂ group, as follows

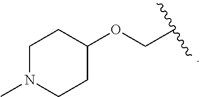 [SMILES: *COC1CCN(C)CC1]

TABLE 1a

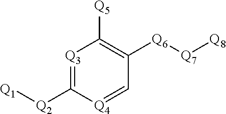

| Example | | | | | | | | |
|---|---|---|---|---|---|---|---|---|
| Number | Q₁ | Q₂ | Q₃ | Q₄ | Q₆ | Q₇ | Q₈ | Q₅ |
| 1001 | 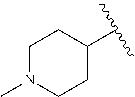 [SMILES: *C1CCN(C)CC1] | O | CH | N | CH₂ | NH | 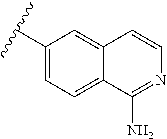 [SMILES: *c1ccc2c(N)nccc2c1] | H |
| 1002 | 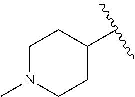 [SMILES: *C1CCN(C)CC1] | O | CH | CH | CH₂ | NH | 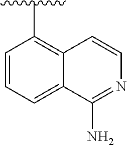 [SMILES: *c1cccc2c(N)nccc12] | H |
| 1003 | 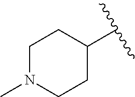 [SMILES: *C1CCN(C)CC1] | O | CH | CH | CH₂ | NH | 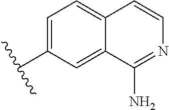 [SMILES: *c1ccc2ccnc(N)c2c1] | H |
| 1004 | 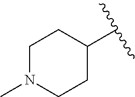 [SMILES: *C1CCN(C)CC1] | O | CH | CH | CH₂ | NH | 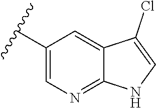 [SMILES: *c1cnc2[nH]cc(Cl)c2c1] | H |
| 1005 | 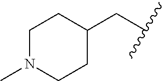 [SMILES: *CC1CCN(C)CC1] | O | CH | N | CH₂ | NH | 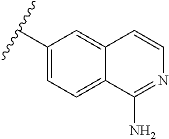 [SMILES: *c1ccc2c(N)nccc2c1] | H |
| 1006 | 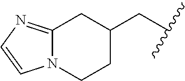 [SMILES: *CC1CCn2ccnc2C1] | O | CH | N | CH₂ | NH | 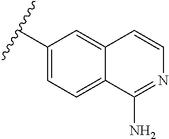 [SMILES: *c1ccc2c(N)nccc2c1] | H |
| 1007 | 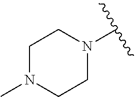 [SMILES: *N1CCN(C)CC1] | CH₂ | CH | CH | CH₂ | NH | 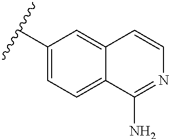 [SMILES: *c1ccc2c(N)nccc2c1] | H |
| 1008 | 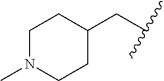 [SMILES: *CC1CCN(C)CC1] | O | CH | N | CH₂ | NH | 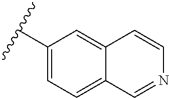 [SMILES: *c1ccc2cnccc2c1] | H |
| 1009 | 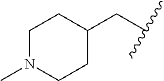 [SMILES: *CC1CCN(C)CC1] | O | CH | N | CH₂ | NH | 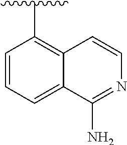 [SMILES: *c1cccc2c(N)nccc12] | H |
| 1010 | 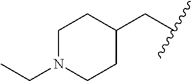 [SMILES: *CC1CCN(CC)CC1] | O | CH | N | CH₂ | NH | 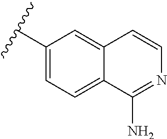 [SMILES: *c1ccc2c(N)nccc2c1] | H |
| 1011 | 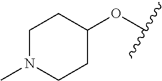 [SMILES: *OC1CCN(C)CC1] | CH₂ | CH | CH | CH₂ | O | 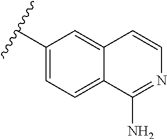 [SMILES: *c1ccc2c(N)nccc2c1] | H |
| 1012 | 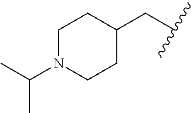 [SMILES: *CC1CCN(C(C)C)CC1] | O | CH | N | CH₂ | NH | 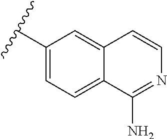 [SMILES: *c1ccc2c(N)nccc2c1] | H |
| 1013 | 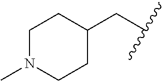 [SMILES: *CC1CCN(C)CC1] | O | N | N | CH₂ | NH | 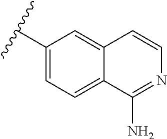 [SMILES: *c1ccc2c(N)nccc2c1] | H |
| 1014 | 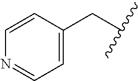 [SMILES: *Cc1ccncc1] | O | CH | N | CH₂ | NH | 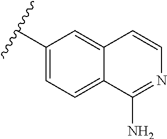 [SMILES: *c1ccc2c(N)nccc2c1] | H |
| 1015 | 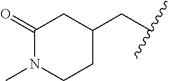 [SMILES: *CC1CCN(C)C(=O)C1] | O | CH | N | CH₂ | NH | 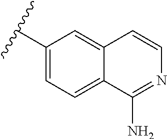 [SMILES: *c1ccc2c(N)nccc2c1] | H |
| 1016 | 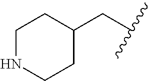 [SMILES: *CC1CCNCC1] | O | CH | N | CH₂ | NH | 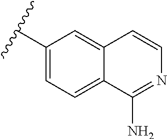 [SMILES: *c1ccc2c(N)nccc2c1] | H |
| 1017 | 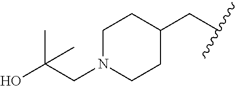 [SMILES: *CC1CCN(CC(C)(C)O)CC1] | O | CH | N | CH₂ | NH | 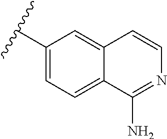 [SMILES: *c1ccc2c(N)nccc2c1] | H |
| 1018 | 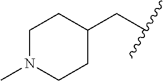 [SMILES: *CC1CCN(C)CC1] | O | N | CH | CH₂ | CH₂ | 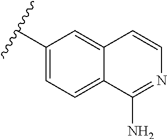 [SMILES: *c1ccc2c(N)nccc2c1] | H |
| 1019 | 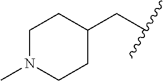 [SMILES: *CC1CCN(C)CC1] | O | N | CH | CH₂ | CH₂ | 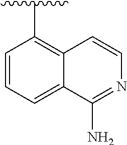 [SMILES: *c1cccc2c(N)nccc12] | H |
| 1020 | 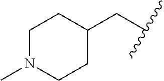 [SMILES: *CC1CCN(C)CC1] | O | CH | N | CH₂ | NH | 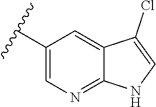 [SMILES: *c1cnc2[nH]cc(Cl)c2c1] | H |
| 1021 | 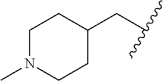 [SMILES: *CC1CCN(C)CC1] | O | CH | N | CH₂ | NH | 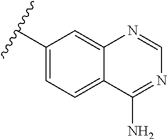 [SMILES: *c1ccc2c(N)ncnc2c1] | H |
| 1022 | 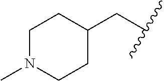 [SMILES: *CC1CCN(C)CC1] | O | CH | N | CH₂ | NH | 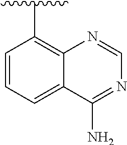 [SMILES: *c1cccc2c(N)ncnc12] | H |
| 1023 | 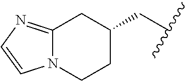 [SMILES: *C[C@@H]1CCn2ccnc2C1] | O | CH | N | CH₂ | NH | 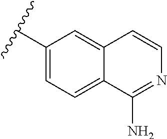 [SMILES: *c1ccc2c(N)nccc2c1] | H |
| 1024 | 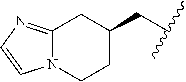 [SMILES: *C[C@H]1CCn2ccnc2C1] | O | CH | N | CH₂ | NH | 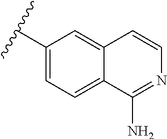 [SMILES: *c1ccc2c(N)nccc2c1] | H |
| 1025 | 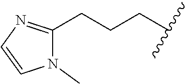 [SMILES: *CCCc1nccn1C] | O | CH | N | CH₂ | NH | 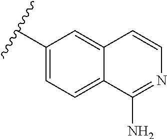 [SMILES: *c1ccc2c(N)nccc2c1] | H |
| 1026 | 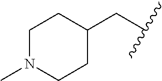 [SMILES: *CC1CCN(C)CC1] | O | CH | N | CH₂ | NH | 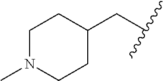 [SMILES: *CC1CCN(C)CC1] | H |
| 1027 | 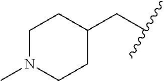 [SMILES: *CC1CCN(C)CC1] | O | N | CH | CH₂ | NH | 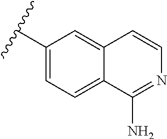 [SMILES: *c1ccc2c(N)nccc2c1] | OCH₃ |
| 1028 | 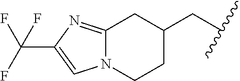 [SMILES: *CC1CCn2cc(C(F)(F)F)nc2C1] | O | CH | N | CH₂ | NH | 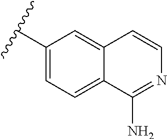 [SMILES: *c1ccc2c(N)nccc2c1] | H |
| 1029 | 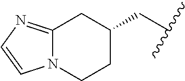 [SMILES: *C[C@@H]1CCn2ccnc2C1] | O | CH | N | CH₂ | NH | 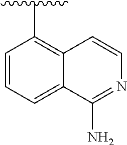 [SMILES: *c1cccc2c(N)nccc12] | H |
| 1030 | 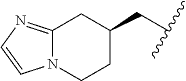 [SMILES: *C[C@H]1CCn2ccnc2C1] | O | CH | N | CH₂ | NH | 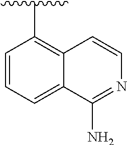 [SMILES: *c1cccc2c(N)nccc12] | H |
| 1031 | 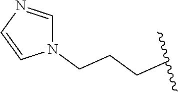 [SMILES: *CCCn1ccnc1] | O | CH | N | CH₂ | NH | 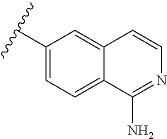 [SMILES: *c1ccc2c(N)nccc2c1] | H |
| 1032 | 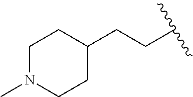 [SMILES: *CCC1CCN(C)CC1] | O | CH | N | CH₂ | NH | 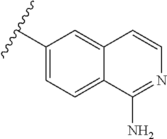 [SMILES: *c1ccc2c(N)nccc2c1] | H |
| 1033 | 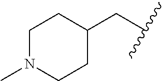 [SMILES: *CC1CCN(C)CC1] | NH | CH | N | CH₂ | NH | 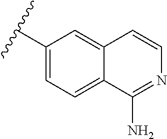 [SMILES: *c1ccc2c(N)nccc2c1] | H |
| 1034 | 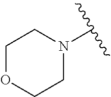 [SMILES: *N1CCOCC1] | CH | CH | CH | CH₂ | NH | 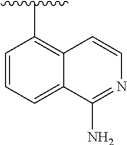 [SMILES: *c1cccc2c(N)nccc12] | F |
| 1035 | 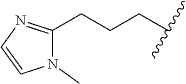 [SMILES: *CCCc1nccn1C] | O | N | N | CH₂ | NH | 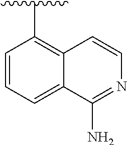 [SMILES: *c1cccc2c(N)nccc12] | H |
| 1036 | 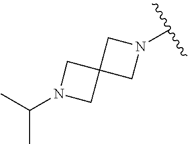 [SMILES: *N1CC2(C1)CN(C(C)C)C2] | CH₂ | CH | CH | CH₂ | NH | 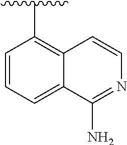 [SMILES: *c1cccc2c(N)nccc12] | F |
| 1037 | 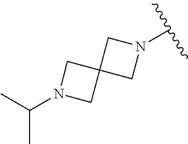 [SMILES: *N1CC2(C1)CN(C(C)C)C2] | CH₂ | CH | CH | CH₂ | NH | 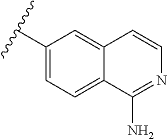 [SMILES: *c1ccc2c(N)nccc2c1] | F |
| 1038 | 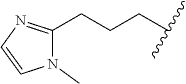 [SMILES: *CCCc1nccn1C] | NH | N | N | CH₂ | NH | 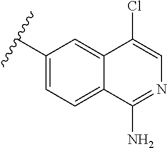 [SMILES: *c1ccc2c(N)ncc(Cl)c2c1] | H |
| 1039 | 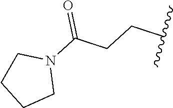 [SMILES: *CCC(=O)N1CCCC1] | NH | N | N | CH₂ | NH | 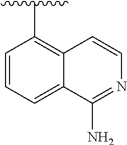 [SMILES: *c1cccc2c(N)nccc12] | H |
| 1040 | 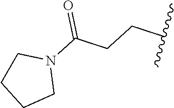 [SMILES: *CCC(=O)N1CCCC1] | NH | N | N | CH₂ | NH | 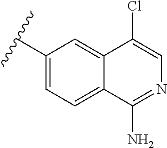 [SMILES: *c1ccc2c(N)ncc(Cl)c2c1] | H |
| 1041 | 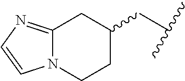 [SMILES: *CC1CCn2ccnc2C1] | O | N | CH | CH₂ | NH | 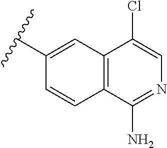 [SMILES: *c1ccc2c(N)ncc(Cl)c2c1] | H |
| 1042 | 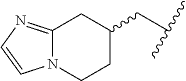 [SMILES: *CC1CCn2ccnc2C1] | O | N | N | CH₂ | NH | 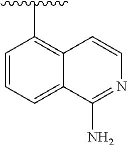 [SMILES: *c1cccc2c(N)nccc12] | H |
| 1043 | 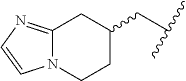 [SMILES: *CC1CCn2ccnc2C1] | NH | N | N | CH₂ | NH | 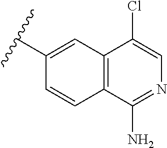 [SMILES: *c1ccc2c(N)ncc(Cl)c2c1] | H |
| 1044 | 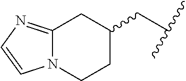 [SMILES: *CC1CCn2ccnc2C1] | NH | N | CH | CH₂ | NH | 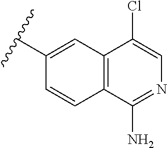 [SMILES: *c1ccc2c(N)ncc(Cl)c2c1] | H |
| 1045 | 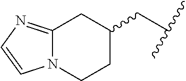 [SMILES: *CC1CCn2ccnc2C1] | NH | N | CH | C(CH₃)₂ | NH | 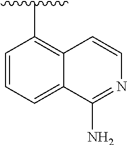 [SMILES: *c1cccc2c(N)nccc12] | H |
| 1046 | 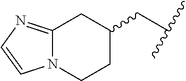 [SMILES: *CC1CCn2ccnc2C1] | NH | N | CH | CHCH₃ | NH | 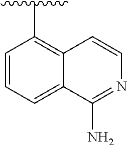 [SMILES: *c1cccc2c(N)nccc12] | H |
| 1047 | 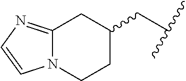 [SMILES: *CC1CCn2ccnc2C1] | NH | N | CH | 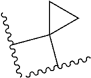 [SMILES: *C1(*)CC1] | NH | 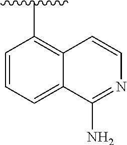 [SMILES: *c1cccc2c(N)nccc12] | H |
| 1048 | 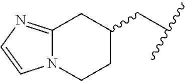 [SMILES: *CC1CCn2ccnc2C1] | NH | N | CH | 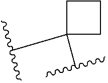 [SMILES: *C1(*)CCC1] | NH | 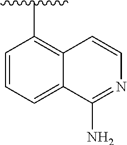 [SMILES: *c1cccc2c(N)nccc12] | H |
| 1049 | 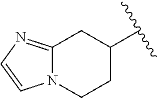 [SMILES: *C1CCn2ccnc2C1] | O | N | CH | CH₂ | NH | 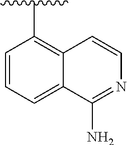 [SMILES: *c1cccc2c(N)nccc12] | H |
| 1050 | 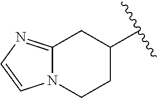 [SMILES: *C1CCn2ccnc2C1] | O | N | CH | CH₂ | NH | 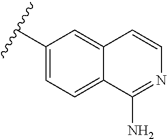 [SMILES: *c1ccc2c(N)nccc2c1] | H |
| 1051 | 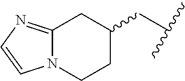 [SMILES: *CC1CCn2ccnc2C1] | NH | N | N | CH₂ | NH | 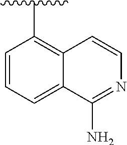 [SMILES: *c1cccc2c(N)nccc12] | H |
| 1052 | 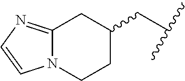 [SMILES: *CC1CCn2ccnc2C1] | O | N | N | CH₂ | NH | 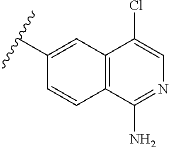 [SMILES: *c1ccc2c(N)ncc(Cl)c2c1] | H |
| 1053 | 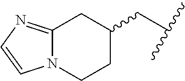 [SMILES: *CC1CCn2ccnc2C1] | NH | N | CH | CH₂ | NH | 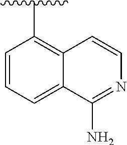 [SMILES: *c1cccc2c(N)nccc12] | H |
| 1054 | 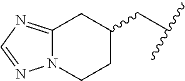 [SMILES: *CC1CCn2ncnc2C1] | O | N | N | CH₂ | NH | 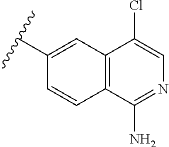 [SMILES: *c1ccc2c(N)ncc(Cl)c2c1] | H |
| 1055 | 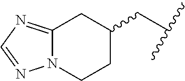 [SMILES: *CC1CCn2ncnc2C1] | O | N | N | CH₂ | NH | 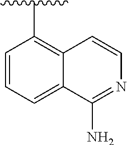 [SMILES: *c1cccc2c(N)nccc12] | H |
| 1056 | 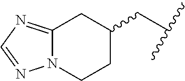 [SMILES: *CC1CCn2ncnc2C1] | NH | N | CH | CH₂ | NH | 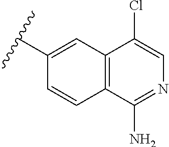 [SMILES: *c1ccc2c(N)ncc(Cl)c2c1] | H |
| 1057 | 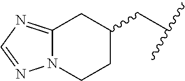 [SMILES: *CC1CCn2ncnc2C1] | O | N | N | CH₂ | NH | 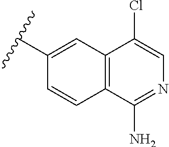 [SMILES: *c1ccc2c(N)ncc(Cl)c2c1] | H |
| 1058 | 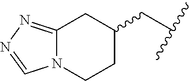 [SMILES: *CC1CCn2cnnc2C1] | NH | N | CH | CH₂ | NH | 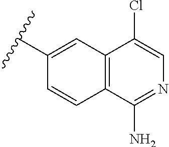 [SMILES: *c1ccc2c(N)ncc(Cl)c2c1] | H |
| 1059 | 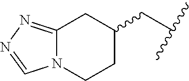 [SMILES: *CC1CCn2cnnc2C1] | O | N | N | CH₂ | NH | 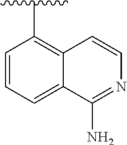 [SMILES: *c1cccc2c(N)nccc12] | H |
| 1060 | 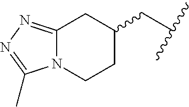 [SMILES: *CC1CCn2c(C)nnc2C1] | O | N | N | CH₂ | NH | 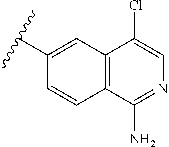 [SMILES: *c1ccc2c(N)ncc(Cl)c2c1] | H |
| 1061 | 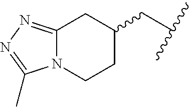 [SMILES: *CC1CCn2c(C)nnc2C1] | NH | N | CH | CH₂ | NH | 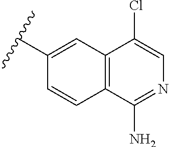 [SMILES: *c1ccc2c(N)ncc(Cl)c2c1] | H |
| 1062 | 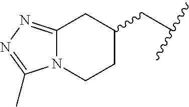 [SMILES: *CC1CCn2c(C)nnc2C1] | O | N | N | CH₂ | NH | 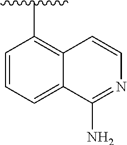 [SMILES: *c1cccc2c(N)nccc12] | H |
| 1063 | 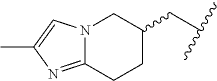 [SMILES: *CC1CCc2nc(C)cn2C1] | O | N | N | CH₂ | NH | 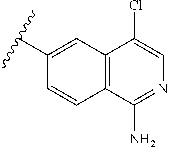 [SMILES: *c1ccc2c(N)ncc(Cl)c2c1] | H |
| 1064 | 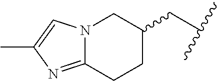 [SMILES: *CC1CCc2nc(C)cn2C1] | O | N | N | CH₂ | NH | 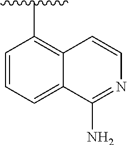 [SMILES: *c1cccc2c(N)nccc12] | H |
| 1065 | 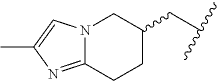 [SMILES: *CC1CCc2nc(C)cn2C1] | NH | N | CH | CH₂ | NH | 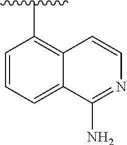 [SMILES: *c1cccc2c(N)nccc12] | H |
| 1068 | 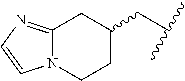 [SMILES: *CC1CCn2ccnc2C1] | NH | N | CH | CH₂ | NH | 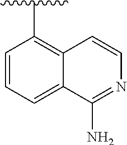 [SMILES: *c1cccc2c(N)nccc12] | H |
| 1069 | 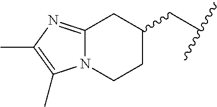 [SMILES: *CC1CCn2c(nc(C)c2C)C1] | O | N | CH | CH₂ | NH | 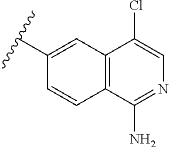 [SMILES: *c1ccc2c(N)ncc(Cl)c2c1] | H |
| 1070 | 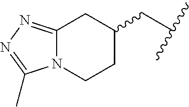 [SMILES: *CC1CCn2c(C)nnc2C1] | O | N | CH | CH₂ | NH | 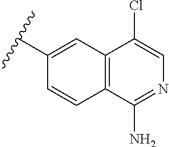 [SMILES: *c1ccc2c(N)ncc(Cl)c2c1] | H |
| 1071 | 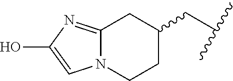 [SMILES: *CC1CCn2cc(O)nc2C1] | O | N | CH | CH₂ | NH | 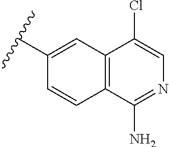 [SMILES: *c1ccc2c(N)ncc(Cl)c2c1] | H |
| 1072 | 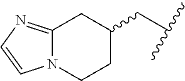 [SMILES: *CC1CCn2ccnc2C1] | NH | N | CH | SO₂ | NH | 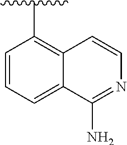 [SMILES: *c1cccc2c(N)nccc12] | H |
| 1073 | 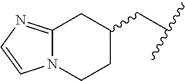 [SMILES: *CC1CCn2ccnc2C1] | NH | N | CH | CH₂ | NH | 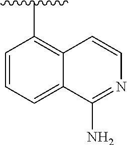 [SMILES: *c1cccc2c(N)nccc12] | H |
| 1074 | 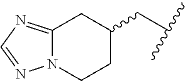 [SMILES: *CC1CCn2ncnc2C1] | O | N | N | CH₂ | NH | 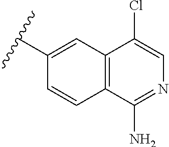 [SMILES: *c1ccc2c(N)ncc(Cl)c2c1] | H |
| 1075 | 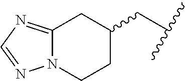 [SMILES: *CC1CCn2ncnc2C1] | O | N | N | CH₂ | NH | 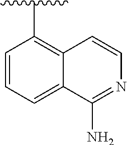 [SMILES: *c1cccc2c(N)nccc12] | H |
| 1076 | 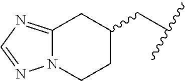 [SMILES: *CC1CCn2ncnc2C1] | NH | N | CH | CH₂ | NH | 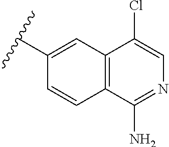 [SMILES: *c1ccc2c(N)ncc(Cl)c2c1] | H |
| 1077 | 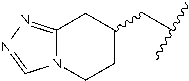 [SMILES: *CC1CCn2cnnc2C1] | O | N | N | CH₂ | NH | 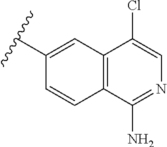 [SMILES: *c1ccc2c(N)ncc(Cl)c2c1] | H |
| 1078 | 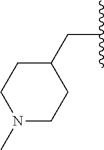 [SMILES: *CC1CCN(C)CC1] | NH | N | CH | CH₂ | NH | 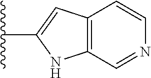 [SMILES: *c1cc2ccncc2[nH]1] | H |
| 1079 | 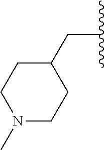 [SMILES: *CC1CCN(C)CC1] | NH | N | CH | CH₂ | NH | 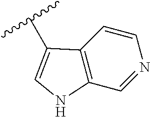 [SMILES: *c1c[nH]c2cnccc12] | H |
| 1080 | 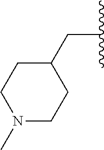 [SMILES: *CC1CCN(C)CC1] | NH | N | CH | CH₂ | NH | 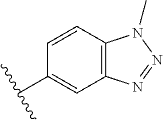 [SMILES: *c1ccc2c(c1)nnn2C] | H |
| 1081 | 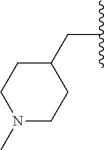 [SMILES: *CC1CCN(C)CC1] | NH | N | CH | CH₂ | NH | 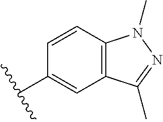 [SMILES: *c1ccc2c(c1)c(C)nn2C] | H |
| 1082 | 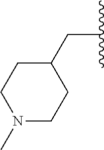 [SMILES: *CC1CCN(C)CC1] | NH | N | CH | CH₂ | NH | 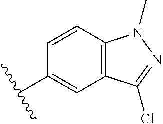 [SMILES: *c1ccc2c(c1)c(Cl)nn2C] | H |
| 1083 | 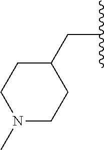 [SMILES: *CC1CCN(C)CC1] | NH | N | CH | CH₂ | NH | 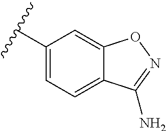 [SMILES: *c1ccc2c(N)noc2c1] | H |
| 1084 | 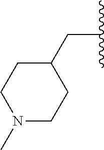 [SMILES: *CC1CCN(C)CC1] | NH | N | CH | CH₂ | NH | 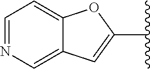 [SMILES: *c1cc2cnccc2o1] | H |
| 1085 | 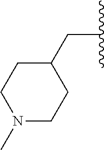 [SMILES: *CC1CCN(C)CC1] | NH | N | CH | CH₂ | NH | 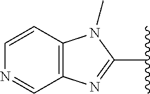 [SMILES: *c1nc2cnccc2n1C] | H |
| 1086 | 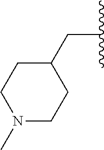 [SMILES: *CC1CCN(C)CC1] | NH | N | CH | CH₂ | NH | 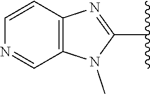 [SMILES: *c1nc2ccncc2n1C] | H |
| 1087 | 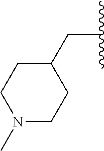 [SMILES: *CC1CCN(C)CC1] | NH | N | CH | CH₂ | NH | 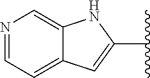 [SMILES: *c1cc2ccncc2[nH]1] | H |
| 1088 | 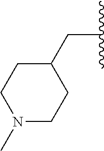 [SMILES: *CC1CCN(C)CC1] | NH | N | CH | CH₂ | NH | 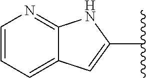 [SMILES: *c1cc2cccnc2[nH]1] | H |
| 1089 | 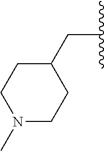 [SMILES: *CC1CCN(C)CC1] | NH | N | CH | CH₂ | NH | 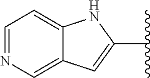 [SMILES: *c1cc2cnccc2[nH]1] | H |
| 1090 | 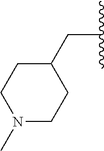 [SMILES: *CC1CCN(C)CC1] | NH | N | CH | CH₂ | NH | 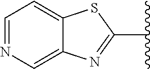 [SMILES: *c1nc2cnccc2s1] | H |
| 1091 | 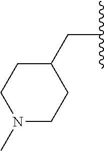 [SMILES: *CC1CCN(C)CC1] | NH | N | CH | CH₂ | NH | 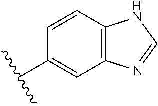 [SMILES: *c1ccc2[nH]cnc2c1] | H |
| 1092 | 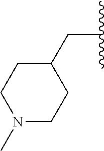 [SMILES: *CC1CCN(C)CC1] | NH | N | CH | CH₂ | NH | 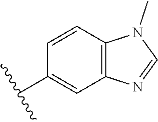 [SMILES: *c1ccc2c(c1)ncn2C] | H |
| 1093 | 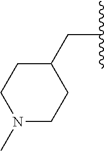 [SMILES: *CC1CCN(C)CC1] | NH | N | CH | CH₂ | NH | 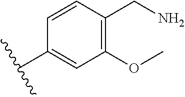 [SMILES: *c1ccc(CN)c(OC)c1] | H |
| 1094 | 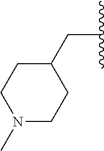 [SMILES: *CC1CCN(C)CC1] | NH | N | CH | CH₂ | NH | 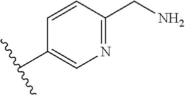 [SMILES: *c1ccc(CN)nc1] | H |
| 1095 | 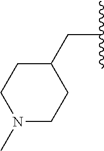 [SMILES: *CC1CCN(C)CC1] | NH | N | CH | CH₂ | NH | 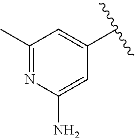 [SMILES: *c1cc(C)nc(N)c1] | H |
| 1096 | 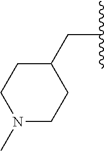 [SMILES: *CC1CCN(C)CC1] | NH | N | CH | CH₂ | NH | 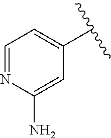 [SMILES: *c1ccnc(N)c1] | H |
| 1097 | 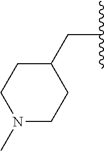 [SMILES: *CC1CCN(C)CC1] | NH | N | CH | CH₂ | NH | 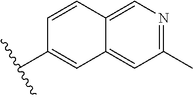 [SMILES: *c1ccc2cnc(C)cc2c1] | H |
| 1098 | 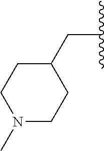 [SMILES: *CC1CCN(C)CC1] | NH | N | CH | CH₂ | NH | 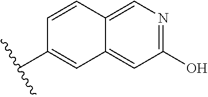 [SMILES: *c1ccc2cnc(O)cc2c1] | H |
| 1099 | 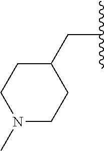 [SMILES: *CC1CCN(C)CC1] | NH | N | CH | CH₂ | NH | 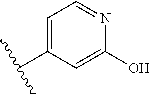 [SMILES: *c1ccnc(O)c1] | H |
| 1100 | 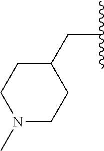 [SMILES: *CC1CCN(C)CC1] | NH | N | CH | CH₂ | NH | 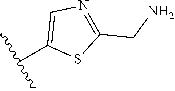 [SMILES: *c1cnc(CN)s1] | H |
| 1101 | 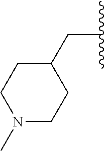 [SMILES: *CC1CCN(C)CC1] | NH | N | CH | CH₂ | NH | 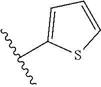 [SMILES: *c1cccs1] | H |
| 1102 | 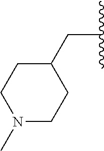 [SMILES: *CC1CCN(C)CC1] | NH | N | CH | CH₂ | NH | 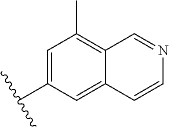 [SMILES: *c1cc(C)c2cnccc2c1] | H |
| 1103 | 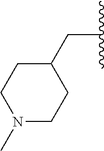 [SMILES: *CC1CCN(C)CC1] | NH | N | CH | CH₂ | NH | 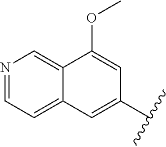 [SMILES: *c1cc(OC)c2cnccc2c1] | H |
| 1104 | 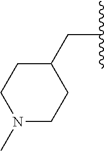 [SMILES: *CC1CCN(C)CC1] | NH | N | CH | CH₂ | NH | 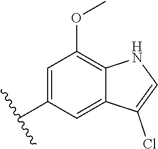 [SMILES: *c1cc(OC)c2[nH]cc(Cl)c2c1] | H |
| 1105 | 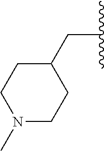 [SMILES: *CC1CCN(C)CC1] | NH | N | CH | CH₂ | NH | 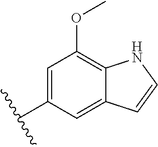 [SMILES: *c1cc(OC)c2[nH]ccc2c1] | H |
| 1106 | 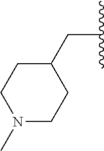 [SMILES: *CC1CCN(C)CC1] | NH | N | CH | CH₂ | NH | 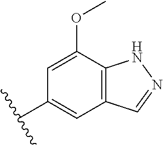 [SMILES: *c1cc(OC)c2[nH]ncc2c1] | H |
| 1107 | 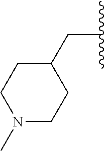 [SMILES: *CC1CCN(C)CC1] | NH | N | CH | CH₂ | NH | 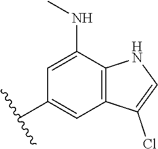 [SMILES: *c1cc(NC)c2[nH]cc(Cl)c2c1] | H |
| 1108 | 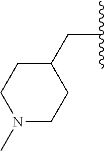 [SMILES: *CC1CCN(C)CC1] | NH | N | CH | CH₂ | NH | 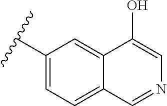 [SMILES: *c1ccc2cncc(O)c2c1] | H |
| 1109 | 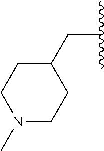 [SMILES: *CC1CCN(C)CC1] | NH | N | CH | CH₂ | NH | 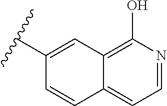 [SMILES: *c1ccc2ccnc(O)c2c1] | H |
| 1110 | 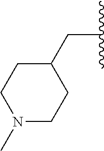 [SMILES: *CC1CCN(C)CC1] | NH | N | CH | CH₂ | NH | 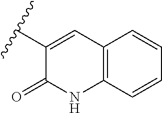 [SMILES: *c1cc2ccccc2[nH]c1=O] | H |
| 1111 | 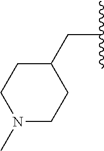 [SMILES: *CC1CCN(C)CC1] | NH | N | CH | CH₂ | NH | 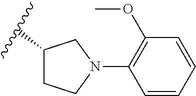 [SMILES: *[C@H]1CCN(c2ccccc2OC)C1] | H |
| 1112 | 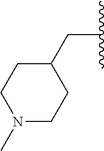 [SMILES: *CC1CCN(C)CC1] | NH | N | CH | CH₂ | NH | 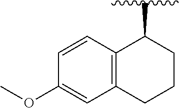 [SMILES: *[C@H]1CCCc2cc(OC)ccc21] | H |
| 1113 | 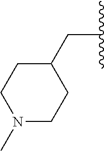 [SMILES: *CC1CCN(C)CC1] | NH | N | CH | CH₂ | NH | 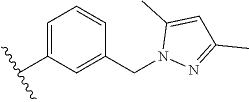 [SMILES: *c1cccc(Cn2nc(C)cc2C)c1] | H |
| 1114 | 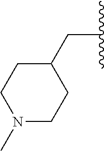 [SMILES: *CC1CCN(C)CC1] | NH | N | CH | CH₂ | NH | 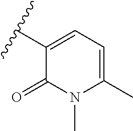 [SMILES: *c1ccc(C)n(C)c1=O] | H |
| 1115 | 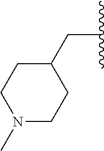 [SMILES: *CC1CCN(C)CC1] | NH | N | CH | CH₂ | NH | 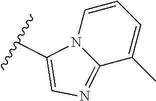 [SMILES: *c1cnc2c(C)cccn12] | H |
| 1116 | 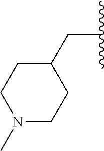 [SMILES: *CC1CCN(C)CC1] | NH | N | CH | CH₂ | NH | 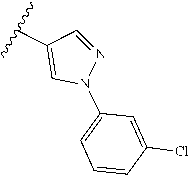 [SMILES: *c1cnn(-c2cccc(Cl)c2)c1] | H |
| 1117 | 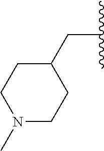 [SMILES: *CC1CCN(C)CC1] | NH | N | CH | CH₂ | NH | 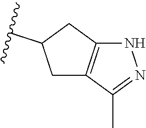 [SMILES: *C1Cc2[nH]nc(C)c2C1] | H |
| 1118 | 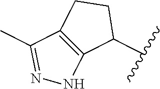 [SMILES: *C1CCc2c(C)n[nH]c21] | NH | N | CH | CH₂ | NH | 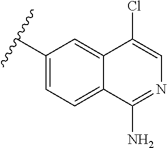 [SMILES: *c1ccc2c(N)ncc(Cl)c2c1] | H |
| 1119 | 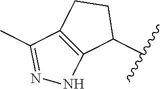 [SMILES: *C1CCc2c(C)n[nH]c21] | NH | N | CH | CH₂ | NH | 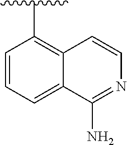 [SMILES: *c1cccc2c(N)nccc12] | H |
| 1120 | 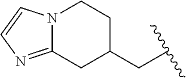 [SMILES: *CC1CCn2ccnc2C1] | NH | N | CH | CH₂ | NH | 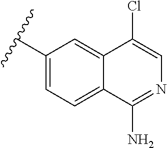 [SMILES: *c1ccc2c(N)ncc(Cl)c2c1] | OCH₃ |
| 1121 | 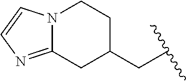 [SMILES: *CC1CCn2ccnc2C1] | NH | N | CH | CH₂ | NH | 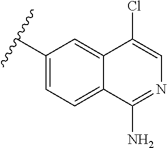 [SMILES: *c1ccc2c(N)ncc(Cl)c2c1] | OiPr |
| 1122 | 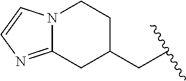 [SMILES: *CC1CCn2ccnc2C1] | O | N | CH | CH₂ | NH | 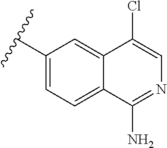 [SMILES: *c1ccc2c(N)ncc(Cl)c2c1] | OCH₃ |
| 1123 | 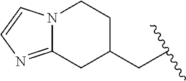 [SMILES: *CC1CCn2ccnc2C1] | O | N | CH | CH₂ | NH | 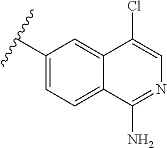 [SMILES: *c1ccc2c(N)ncc(Cl)c2c1] | CH₃ |
| 1124 | 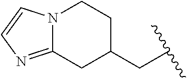 [SMILES: *CC1CCn2ccnc2C1] | NH | N | CH | CH₂ | NH | 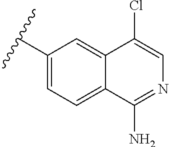 [SMILES: *c1ccc2c(N)ncc(Cl)c2c1] | CH₃ |
| 1125 | 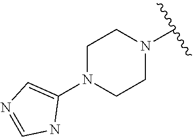 [SMILES: *N1CCN(c2cnc[nH]2)CC1] | absent | N | CH | CH₂ | NH | 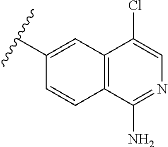 [SMILES: *c1ccc2c(N)ncc(Cl)c2c1] | H |
| 1126 | 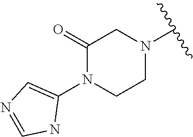 [SMILES: *N1CCN(c2cnc[nH]2)C(=O)C1] | absent | N | CH | CH₂ | NH | 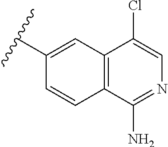 [SMILES: *c1ccc2c(N)ncc(Cl)c2c1] | H |
| 1127 | 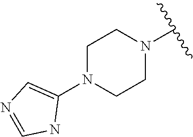 [SMILES: *N1CCN(c2cnc[nH]2)CC1] | absent | N | CH | CH₂ | NH | 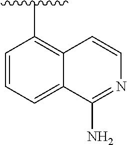 [SMILES: *c1cccc2c(N)nccc12] | H |
| 1128 | 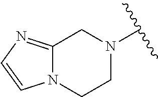 [SMILES: *N1CCn2ccnc2C1] | absent | N | CH | CH₂ | NH | 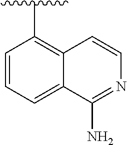 [SMILES: *c1cccc2c(N)nccc12] | H |
| 1129 | 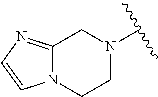 [SMILES: *N1CCn2ccnc2C1] | absent | N | CH | CH₂ | NH | 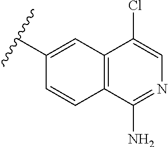 [SMILES: *c1ccc2c(N)ncc(Cl)c2c1] | H |
| 1130 | 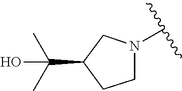 [SMILES: *N1CC[C@@H](C(C)(C)O)C1] | absent | N | CH | CH₂ | NH | 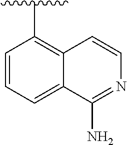 [SMILES: *c1cccc2c(N)nccc12] | H |
| 1131 | 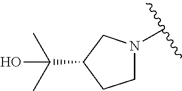 [SMILES: *N1CC[C@H](C(C)(C)O)C1] | absent | N | CH | CH₂ | NH | 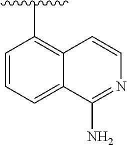 [SMILES: *c1cccc2c(N)nccc12] | H |
| 1132 | 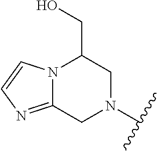 [SMILES: *N1Cc2nccn2C(CO)C1] | absent | N | CH | CH₂ | NH | 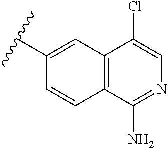 [SMILES: *c1ccc2c(N)ncc(Cl)c2c1] | H |
| 1133 | 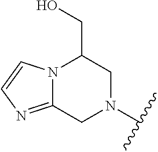 [SMILES: *N1Cc2nccn2C(CO)C1] | absent | N | CH | CH₂ | NH | 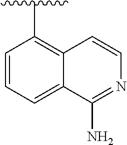 [SMILES: *c1cccc2c(N)nccc12] | H |
| 1134 | 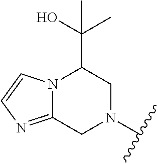 [SMILES: *N1Cc2nccn2C(C(C)(C)O)C1] | absent | N | CH | CH₂ | NH | 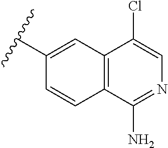 [SMILES: *c1ccc2c(N)ncc(Cl)c2c1] | H |
| 1135 | 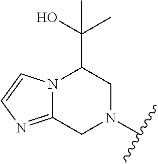 [SMILES: *N1Cc2nccn2C(C(C)(C)O)C1] | absent | N | CH | CH₂ | NH | 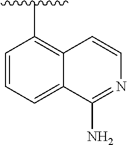 [SMILES: *c1cccc2c(N)nccc12] | H |
| 1136 | 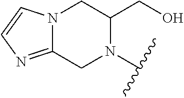 [SMILES: *N1Cc2nccn2CC1CO] | absent | N | CH | CH₂ | NH | 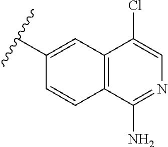 [SMILES: *c1ccc2c(N)ncc(Cl)c2c1] | H |
| 1137 | 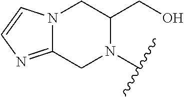 [SMILES: *N1Cc2nccn2CC1CO] | absent | N | CH | CH₂ | NH | 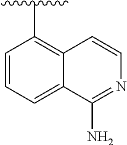 [SMILES: *c1cccc2c(N)nccc12] | H |
| 1138 | 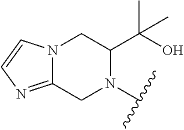 [SMILES: *N1Cc2nccn2CC1C(C)(C)O] | absent | N | CH | CH₂ | NH | 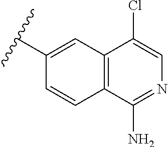 [SMILES: *c1ccc2c(N)ncc(Cl)c2c1] | H |
| 1139 | 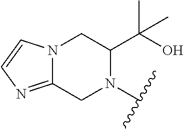 [SMILES: *N1Cc2nccn2CC1C(C)(C)O] | absent | N | CH | CH₂ | NH | 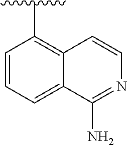 [SMILES: *c1cccc2c(N)nccc12] | H |
| 1140 | 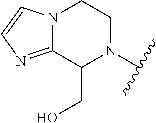 [SMILES: *N1CCn2ccnc2C1CO] | absent | N | CH | CH₂ | NH | 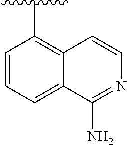 [SMILES: *c1cccc2c(N)nccc12] | H |
| 1141 | 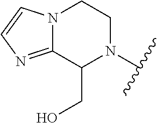 [SMILES: *N1CCn2ccnc2C1CO] | absent | N | CH | CH₂ | NH | 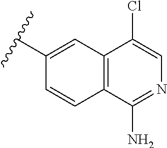 [SMILES: *c1ccc2c(N)ncc(Cl)c2c1] | H |
| 1142 | 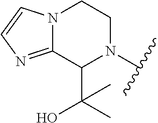 [SMILES: *N1CCn2ccnc2C1C(C)(C)O] | absent | N | CH | CH₂ | NH | 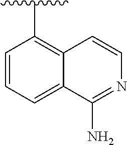 [SMILES: *c1cccc2c(N)nccc12] | H |
| 1143 | 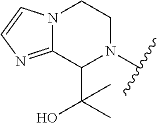 [SMILES: *N1CCn2ccnc2C1C(C)(C)O] | absent | N | CH | CH₂ | NH | 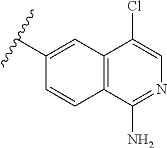 [SMILES: *c1ccc2c(N)ncc(Cl)c2c1] | H |
| 1144 | 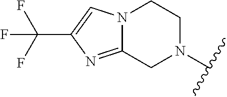 [SMILES: *N1CCn2cc(C(F)(F)F)nc2C1] | absent | N | CH | CH₂ | NH | 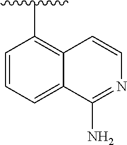 [SMILES: *c1cccc2c(N)nccc12] | H |
| 1145 | 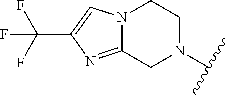 [SMILES: *N1CCn2cc(C(F)(F)F)nc2C1] | absent | N | CH | CH₂ | NH | 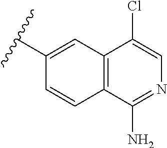 [SMILES: *c1ccc2c(N)ncc(Cl)c2c1] | H |
| 1146 | 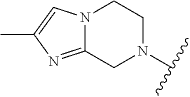 [SMILES: *N1CCn2cc(C)nc2C1] | absent | N | CH | CH₂ | NH | 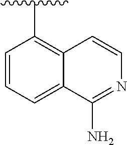 [SMILES: *c1cccc2c(N)nccc12] | H |
| 1147 | 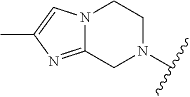 [SMILES: *N1CCn2cc(C)nc2C1] | absent | N | CH | CH₂ | NH | 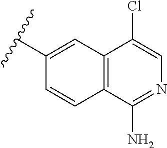 [SMILES: *c1ccc2c(N)ncc(Cl)c2c1] | H |
| 1148 | 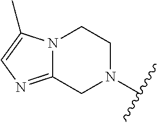 [SMILES: *N1CCn2c(C)cnc2C1] | absent | N | CH | CH₂ | NH | 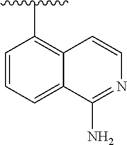 [SMILES: *c1cccc2c(N)nccc12] | H |
| 1149 | 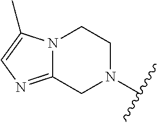 [SMILES: *N1CCn2c(C)cnc2C1] | absent | N | CH | CH₂ | NH | 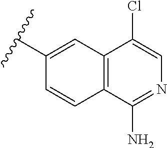 [SMILES: *c1ccc2c(N)ncc(Cl)c2c1] | H |
| 1150 | 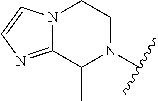 [SMILES: *N1CCn2ccnc2C1C] | absent | N | CH | CH₂ | NH | 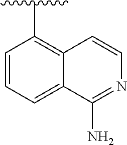 [SMILES: *c1cccc2c(N)nccc12] | H |
| 1151 | 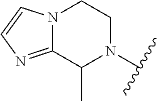 [SMILES: *N1CCn2ccnc2C1C] | absent | N | CH | CH₂ | NH | 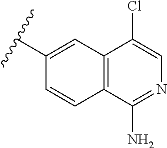 [SMILES: *c1ccc2c(N)ncc(Cl)c2c1] | H |
| 1152 | 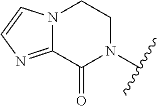 [SMILES: *N1CCn2ccnc2C1=O] | absent | N | CH | CH₂ | NH | 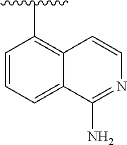 [SMILES: *c1cccc2c(N)nccc12] | H |
| 1153 | 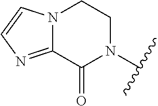 [SMILES: *N1CCn2ccnc2C1=O] | absent | N | CH | CH₂ | NH | 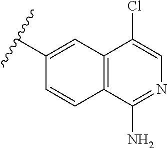 [SMILES: *c1ccc2c(N)ncc(Cl)c2c1] | H |
| 1154 | 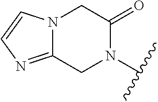 [SMILES: *N1Cc2nccn2CC1=O] | absent | N | CH | CH₂ | NH | 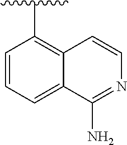 [SMILES: *c1cccc2c(N)nccc12] | H |
| 1155 | 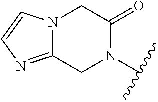 [SMILES: *N1Cc2nccn2CC1=O] | absent | N | CH | CH₂ | NH | 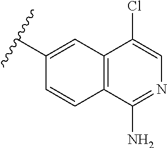 [SMILES: *c1ccc2c(N)ncc(Cl)c2c1] | H |
| 1156 | 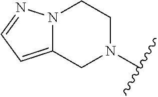 [SMILES: *N1CCn2nccc2C1] | absent | N | CH | CH₂ | NH | 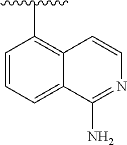 [SMILES: *c1cccc2c(N)nccc12] | H |
| 1157 | 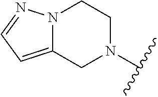 [SMILES: *N1CCn2nccc2C1] | absent | N | CH | CH₂ | NH | 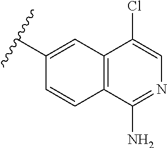 [SMILES: *c1ccc2c(N)ncc(Cl)c2c1] | H |
| 1158 | 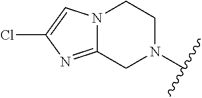 [SMILES: *N1CCn2cc(Cl)nc2C1] | absent | N | CH | CH₂ | NH | 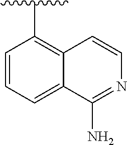 [SMILES: *c1cccc2c(N)nccc12] | H |
| 1159 | 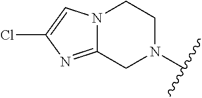 [SMILES: *N1CCn2cc(Cl)nc2C1] | absent | N | CH | CH₂ | NH | 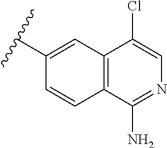 [SMILES: *c1ccc2c(N)ncc(Cl)c2c1] | H |
| 1160 | 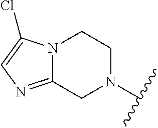 [SMILES: *N1CCn2c(Cl)cnc2C1] | absent | N | CH | CH₂ | NH | 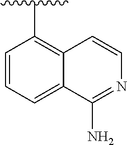 [SMILES: *c1cccc2c(N)nccc12] | H |
| 1161 | 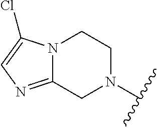 [SMILES: *N1CCn2c(Cl)cnc2C1] | absent | N | CH | CH₂ | NH | 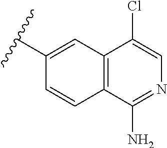 [SMILES: *c1ccc2c(N)ncc(Cl)c2c1] | H |
| 1162 | 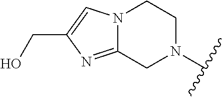 [SMILES: *N1CCn2cc(CO)nc2C1] | absent | N | CH | CH₂ | NH | 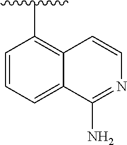 [SMILES: *c1cccc2c(N)nccc12] | H |
| 1163 | 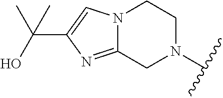 [SMILES: *N1CCn2cc(C(C)(C)O)nc2C1] | absent | N | CH | CH₂ | NH | 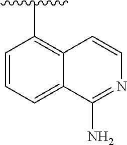 [SMILES: *c1cccc2c(N)nccc12] | H |
| 1164 | 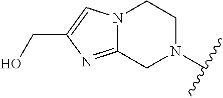 [SMILES: *N1CCn2cc(CO)nc2C1] | absent | N | CH | CH₂ | NH | 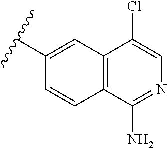 [SMILES: *c1ccc2c(N)ncc(Cl)c2c1] | H |
| 1165 | 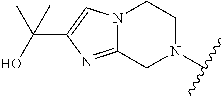 [SMILES: *N1CCn2cc(C(C)(C)O)nc2C1] | absent | N | CH | CH₂ | NH | 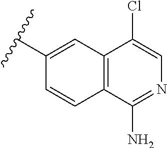 [SMILES: *c1ccc2c(N)ncc(Cl)c2c1] | H |
| 1166 | 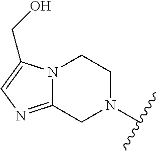 [SMILES: *N1CCn2c(CO)cnc2C1] | absent | N | CH | CH₂ | NH | 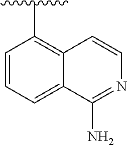 [SMILES: *c1cccc2c(N)nccc12] | H |
| 1167 | 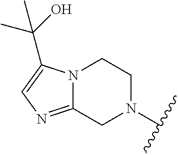 [SMILES: *N1CCn2c(C(C)(C)O)cnc2C1] | absent | N | CH | CH₂ | NH | 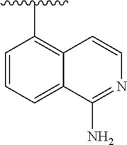 [SMILES: *c1cccc2c(N)nccc12] | H |
| 1168 | 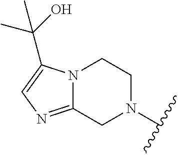 [SMILES: *N1CCn2c(C(C)(C)O)cnc2C1] | absent | N | CH | CH₂ | NH | 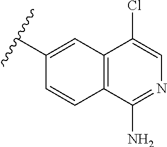 [SMILES: *c1ccc2c(N)ncc(Cl)c2c1] | H |
| 1169 | 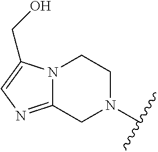 [SMILES: *N1CCn2c(CO)cnc2C1] | absent | N | CH | CH₂ | NH | 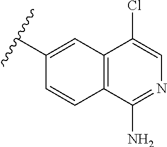 [SMILES: *c1ccc2c(N)ncc(Cl)c2c1] | H |
| 1170 | 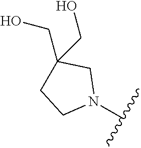 [SMILES: *N1CCC(CO)(CO)C1] | absent | N | CH | CH₂ | NH | 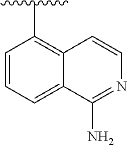 [SMILES: *c1cccc2c(N)nccc12] | H |
| 1171 | 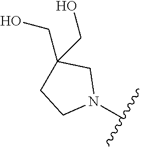 [SMILES: *N1CCC(CO)(CO)C1] | absent | N | CH | CH₂ | NH | 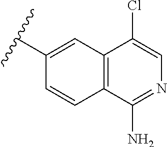 [SMILES: *c1ccc2c(N)ncc(Cl)c2c1] | H |
| 1172 | 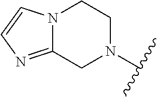 [SMILES: *N1CCn2ccnc2C1] | absent | N | CH | CH₂ | NH | 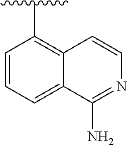 [SMILES: *c1cccc2c(N)nccc12] | OCH₃ |
| 1173 | 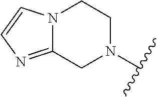 [SMILES: *N1CCn2ccnc2C1] | absent | N | CH | CH₂ | NH | 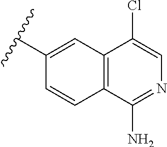 [SMILES: *c1ccc2c(N)ncc(Cl)c2c1] | OCH₃ |
| 1174 | 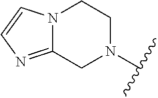 [SMILES: *N1CCn2ccnc2C1] | absent | N | CH | CH₂ | NH | 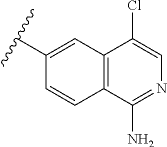 [SMILES: *c1ccc2c(N)ncc(Cl)c2c1] | OiPr |
| 1175 | 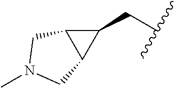 [SMILES: *C[C@@H]1[C@H]2CN(C)C[C@@H]12] | O | CH | N | CH₂ | NH | 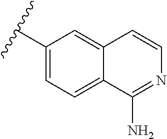 [SMILES: *c1ccc2c(N)nccc2c1] | H |
| 1176 | 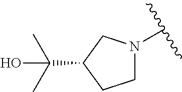 [SMILES: *N1CC[C@H](C(C)(C)O)C1] | absent | N | CH | CH₂ | NH | 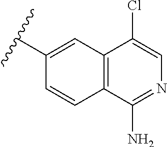 [SMILES: *c1ccc2c(N)ncc(Cl)c2c1] | H |

TABLE 1b

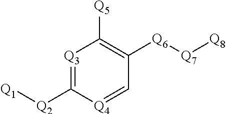

| Ex- | | | | | | | | |
|---|---|---|---|---|---|---|---|---|
| ample | | | | | | | | |
| Num- | | | | | | | | |
| ber | Q₁ | Q₂ | Q₃ | Q₄ | Q₆ | Q₇ | Q₈ | Q₅ |
| 1177 | 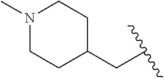 [SMILES: *CC1CCN(C)CC1] | NH | CH | N | CH₂ | NH | 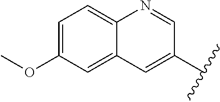 [SMILES: *c1cnc2ccc(OC)cc2c1] | H |
| 1178 | 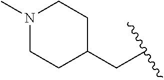 [SMILES: *CC1CCN(C)CC1] | NH | CH | N | CH₂ | NH | 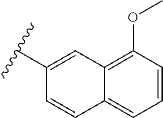 [SMILES: *c1ccc2cccc(OC)c2c1] | H |
| 1180 | 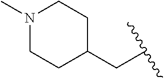 [SMILES: *CC1CCN(C)CC1] | NH | CH | N | CH₂ | NH | 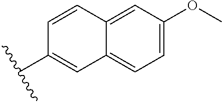 [SMILES: *c1ccc2cc(OC)ccc2c1] | H |
| 1181 | 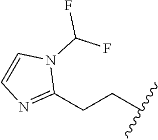 [SMILES: *CCc1nccn1C(F)F] | NH | CH | N | CH₂ | NH | 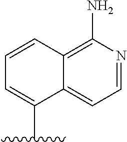 [SMILES: *c1cccc2c(N)nccc12] | H |
| 1182 | 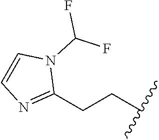 [SMILES: *CCc1nccn1C(F)F] | NH | CH | N | CH₂ | NH | 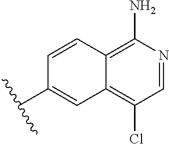 [SMILES: *c1ccc2c(N)ncc(Cl)c2c1] | H |
| 1183 | 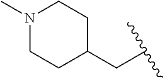 [SMILES: *CC1CCN(C)CC1] | NH | CH | N | CH₂ | NH | 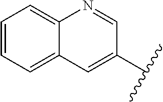 [SMILES: *c1cnc2ccccc2c1] | H |
| 1184 | 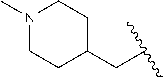 [SMILES: *CC1CCN(C)CC1] | NH | CH | N | CH₂ | NH | 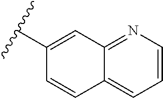 [SMILES: *c1ccc2cccnc2c1] | H |
| 1185 | 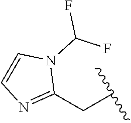 [SMILES: *Cc1nccn1C(F)F] | NH | CH | N | CH₂ | NH | 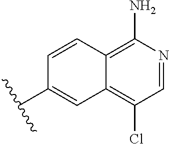 [SMILES: *c1ccc2c(N)ncc(Cl)c2c1] | H |
| 1186 | 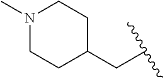 [SMILES: *CC1CCN(C)CC1] | NH | CH | N | CH₂ | NH | 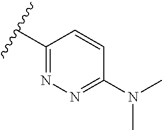 [SMILES: *c1ccc(N(C)C)nn1] | H |
| 1187 | 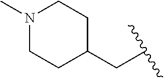 [SMILES: *CC1CCN(C)CC1] | NH | CH | N | CH₂ | NH | 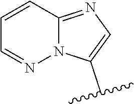 [SMILES: *c1cnc2cccnn12] | H |
| 1188 | 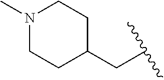 [SMILES: *CC1CCN(C)CC1] | NH | CH | N | CH₂ | NH | 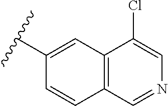 [SMILES: *c1ccc2cncc(Cl)c2c1] | H |
| 1189 | 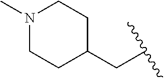 [SMILES: *CC1CCN(C)CC1] | NH | CH | N | CH₂ | NH | 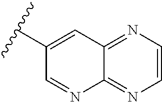 [SMILES: *c1cnc2nccnc2c1] | H |
| 1190 | 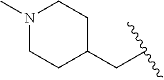 [SMILES: *CC1CCN(C)CC1] | NH | CH | N | CH₂ | NH | 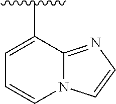 [SMILES: *c1cccn2ccnc12] | H |
| 1191 | 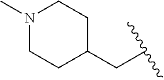 [SMILES: *CC1CCN(C)CC1] | NH | CH | N | CH₂ | NH | 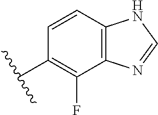 [SMILES: *c1ccc2[nH]cnc2c1F] | H |
| 1192 | 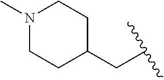 [SMILES: *CC1CCN(C)CC1] | NH | CH | N | CH₂ | NH | 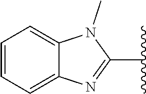 [SMILES: *c1nc2ccccc2n1C] | H |
| 1193 | 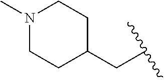 [SMILES: *CC1CCN(C)CC1] | NH | CH | N | CH₂ | NH | 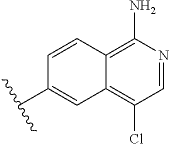 [SMILES: *c1ccc2c(N)ncc(Cl)c2c1] | H |
| 1194 | 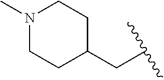 [SMILES: *CC1CCN(C)CC1] | NH | CH | N | CH₂ | NH | 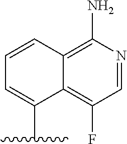 [SMILES: *c1cccc2c(N)ncc(F)c12] | H |
| 1195 | 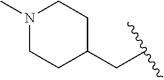 [SMILES: *CC1CCN(C)CC1] | NH | CH | N | CH₂ | NH | 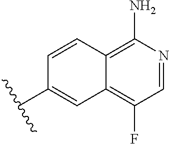 [SMILES: *c1ccc2c(N)ncc(F)c2c1] | H |
| 1196 | 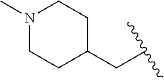 [SMILES: *CC1CCN(C)CC1] | NH | CH | N | CH₂ | NH | 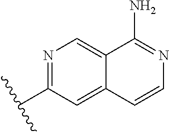 [SMILES: *c1cc2ccnc(N)c2cn1] | H |
| 1197 | 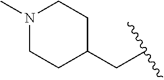 [SMILES: *CC1CCN(C)CC1] | NH | CH | N | CH₂ | NH | 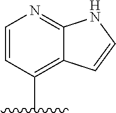 [SMILES: *c1ccnc2[nH]ccc12] | H |
| 1198 | 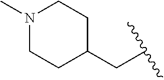 [SMILES: *CC1CCN(C)CC1] | NH | CH | N | CH₂ | NH | 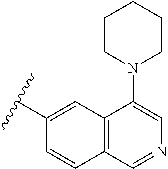 [SMILES: *c1ccc2cncc(N3CCCCC3)c2c1] | H |
| 1199 | 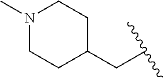 [SMILES: *CC1CCN(C)CC1] | NH | CH | N | CH₂ | NH | 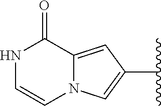 [SMILES: *c1cc2c(=O)[nH]ccn2c1] | H |
| 1200 | 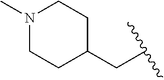 [SMILES: *CC1CCN(C)CC1] | NH | CH | N | CH₂ | NH | 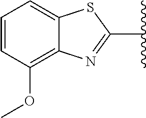 [SMILES: *c1nc2c(OC)cccc2s1] | H |
| 1201 | 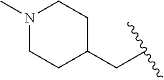 [SMILES: *CC1CCN(C)CC1] | NH | CH | N | CH₂ | NH | 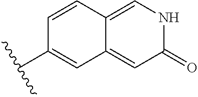 [SMILES: *c1ccc2c[nH]c(=O)cc2c1] | H |
| 1202 | 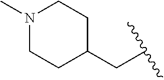 [SMILES: *CC1CCN(C)CC1] | NH | CH | N | CH₂ | NH | 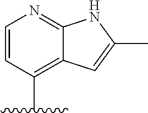 [SMILES: *c1ccnc2[nH]c(C)cc12] | H |
| 1203 | 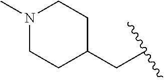 [SMILES: *CC1CCN(C)CC1] | NH | CH | N | CH₂ | NH | 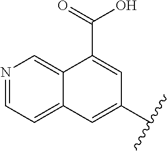 [SMILES: *c1cc(C(=O)O)c2cnccc2c1] | H |
| 1204 | 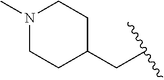 [SMILES: *CC1CCN(C)CC1] | NH | CH | N | CH₂ | NH | 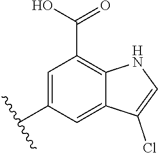 [SMILES: *c1cc(C(=O)O)c2[nH]cc(Cl)c2c1] | H |
| 1205 | 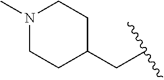 [SMILES: *CC1CCN(C)CC1] | NH | CH | N | CH₂ | NH | 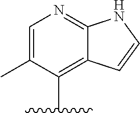 [SMILES: *c1c(C)cnc2[nH]ccc12] | H |
| 1206 | 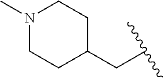 [SMILES: *CC1CCN(C)CC1] | NH | CH | N | CH₂ | NH | 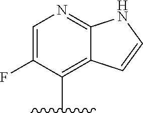 [SMILES: *c1c(F)cnc2[nH]ccc12] | H |
| 1207 | 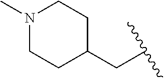 [SMILES: *CC1CCN(C)CC1] | NH | CH | N | CH₂ | NH | 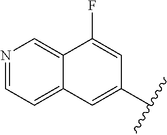 [SMILES: *c1cc(F)c2cnccc2c1] | H |
| 1208 | 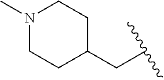 [SMILES: *CC1CCN(C)CC1] | NH | CH | N | CH₂ | NH | 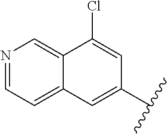 [SMILES: *c1cc(Cl)c2cnccc2c1] | H |
| 1209 | 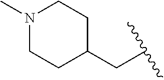 [SMILES: *CC1CCN(C)CC1] | NH | CH | N | CH₂ | NH | 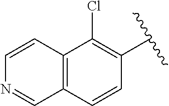 [SMILES: *c1ccc2cnccc2c1Cl] | H |
| 1210 | 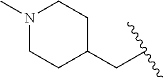 [SMILES: *CC1CCN(C)CC1] | NH | CH | N | CH₂ | NH | 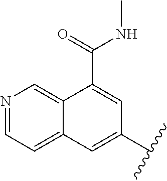 [SMILES: *c1cc(C(=O)NC)c2cnccc2c1] | H |
| 1211 | 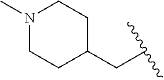 [SMILES: *CC1CCN(C)CC1] | NH | CH | N | CH₂ | NH | 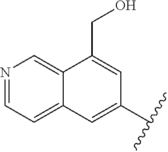 [SMILES: *c1cc(CO)c2cnccc2c1] | H |
| 1212 | 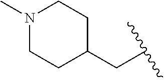 [SMILES: *CC1CCN(C)CC1] | NH | CH | N | CH₂ | NH | 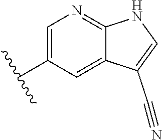 [SMILES: *c1cnc2[nH]cc(C#N)c2c1] | H |
| 1213 | 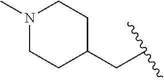 [SMILES: *CC1CCN(C)CC1] | NH | CH | N | CH₂ | NH | 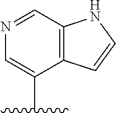 [SMILES: *c1cncc2[nH]ccc12] | H |
| 1214 | 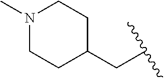 [SMILES: *CC1CCN(C)CC1] | NH | CH | N | CH₂ | NH | 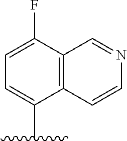 [SMILES: *c1ccc(F)c2cnccc12] | H |
| 1215 | 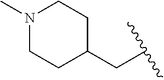 [SMILES: *CC1CCN(C)CC1] | NH | CH | N | CH₂ | NH | 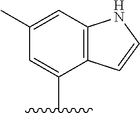 [SMILES: *c1cc(C)cc2[nH]ccc12] | H |
| 1216 | 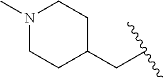 [SMILES: *CC1CCN(C)CC1] | NH | CH | N | CH₂ | NH | 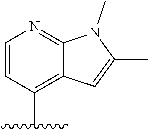 [SMILES: *c1ccnc2c1cc(C)n2C] | H |
| 1217 | 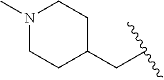 [SMILES: *CC1CCN(C)CC1] | NH | CH | N | CH₂ | NH | 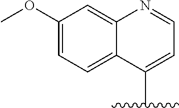 [SMILES: *c1ccnc2cc(OC)ccc12] | H |
| 1218 | 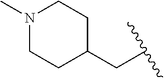 [SMILES: *CC1CCN(C)CC1] | NH | CH | N | CH₂ | NH | 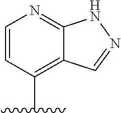 [SMILES: *c1ccnc2[nH]ncc12] | H |
| 1219 | 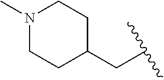 [SMILES: *CC1CCN(C)CC1] | NH | CH | N | CH₂ | NH | 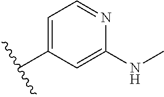 [SMILES: *c1ccnc(NC)c1] | H |
| 1220 | 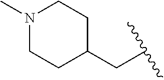 [SMILES: *CC1CCN(C)CC1] | NH | CH | N | CH₂ | NH | 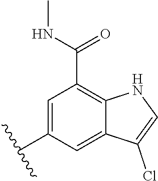 [SMILES: *c1cc(C(=O)NC)c2[nH]cc(Cl)c2c1] | H |
| 1221 | 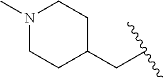 [SMILES: *CC1CCN(C)CC1] | NH | CH | N | CH₂ | NH | 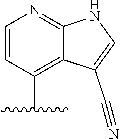 [SMILES: *c1ccnc2[nH]cc(C#N)c12] | H |
| 1222 | 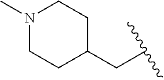 [SMILES: *CC1CCN(C)CC1] | NH | CH | N | CH₂ | NH | 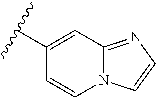 [SMILES: *c1ccn2ccnc2c1] | H |
| 1223 | 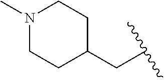 [SMILES: *CC1CCN(C)CC1] | NH | CH | N | CH₂ | NH | 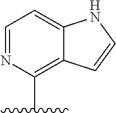 [SMILES: *c1nccc2[nH]ccc12] | H |
| 1224 | 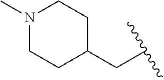 [SMILES: *CC1CCN(C)CC1] | NH | CH | N | CH₂ | NH | 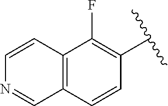 [SMILES: *c1ccc2cnccc2c1F] | H |
| 1225 | 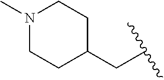 [SMILES: *CC1CCN(C)CC1] | NH | CH | N | CH₂ | NH | 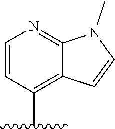 [SMILES: *c1ccnc2c1ccn2C] | H |
| 1226 | 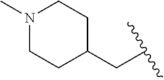 [SMILES: *CC1CCN(C)CC1] | NH | CH | N | CH₂ | NH | 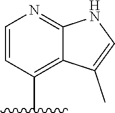 [SMILES: *c1ccnc2[nH]cc(C)c12] | H |
| 1227 | 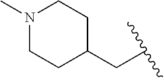 [SMILES: *CC1CCN(C)CC1] | NH | CH | N | CH₂ | NH | 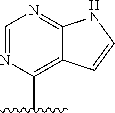 [SMILES: *c1ncnc2[nH]ccc12] | H |
| 1228 | 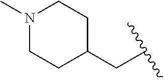 [SMILES: *CC1CCN(C)CC1] | NH | CH | N | CH₂ | NH | 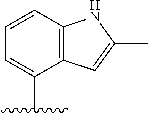 [SMILES: *c1cccc2[nH]c(C)cc12] | H |
| 1229 | 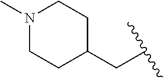 [SMILES: *CC1CCN(C)CC1] | NH | CH | N | CH₂ | NH | 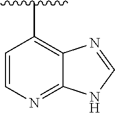 [SMILES: *c1ccnc2[nH]cnc12] | H |
| 1230 | 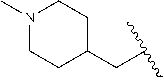 [SMILES: *CC1CCN(C)CC1] | NH | CH | N | CH₂ | NH | 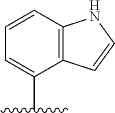 [SMILES: *c1cccc2[nH]ccc12] | H |
| 1231 | 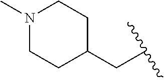 [SMILES: *CC1CCN(C)CC1] | NH | CH | N | CH₂ | NH | 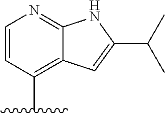 [SMILES: *c1ccnc2[nH]c(C(C)C)cc12] | H |
| 1232 | 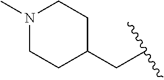 [SMILES: *CC1CCN(C)CC1] | NH | CH | N | CH₂ | NH | 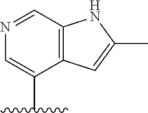 [SMILES: *c1cncc2[nH]c(C)cc12] | H |
| 1233 | 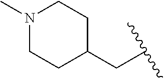 [SMILES: *CC1CCN(C)CC1] | NH | CH | N | CH₂ | NH | 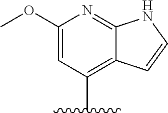 [SMILES: *c1cc(OC)nc2[nH]ccc12] | H |
| 1234 | 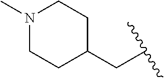 [SMILES: *CC1CCN(C)CC1] | NH | CH | N | CH₂ | NH | 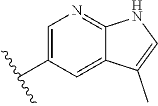 [SMILES: *c1cnc2[nH]cc(C)c2c1] | H |
| 1235 | 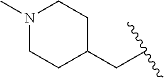 [SMILES: *CC1CCN(C)CC1] | NH | CH | N | CH₂ | NH | 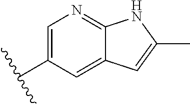 [SMILES: *c1cnc2[nH]c(C)cc2c1] | H |
| 1236 | 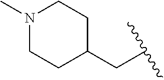 [SMILES: *CC1CCN(C)CC1] | NH | CH | N | CH₂ | NH | 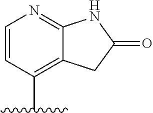 [SMILES: *c1ccnc2c1CC(=O)N2] | H |
| 1237 | 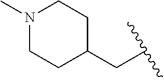 [SMILES: *CC1CCN(C)CC1] | NH | CH | N | CH₂ | NH | 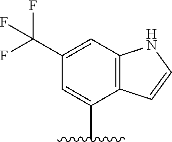 [SMILES: *c1cc(C(F)(F)F)cc2[nH]ccc12] | H |
| 1238 | 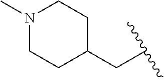 [SMILES: *CC1CCN(C)CC1] | NH | CH | N | CH₂ | NH | 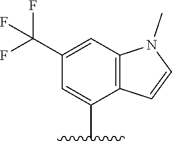 [SMILES: *c1cc(C(F)(F)F)cc2c1ccn2C] | H |
| 1239 | 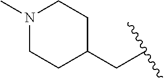 [SMILES: *CC1CCN(C)CC1] | NH | CH | N | CH₂ | NH | 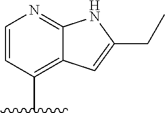 [SMILES: *c1ccnc2[nH]c(CC)cc12] | H |
| 1240 | 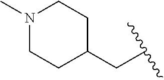 [SMILES: *CC1CCN(C)CC1] | NH | CH | N | CH₂ | NH | 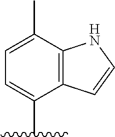 [SMILES: *c1ccc(C)c2[nH]ccc12] | H |
| 1241 | 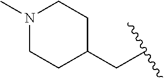 [SMILES: *CC1CCN(C)CC1] | NH | CH | N | CH₂ | NH | 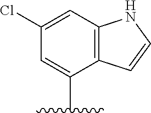 [SMILES: *c1cc(Cl)cc2[nH]ccc12] | H |
| 1242 | 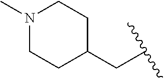 [SMILES: *CC1CCN(C)CC1] | NH | CH | N | CH₂ | NH | 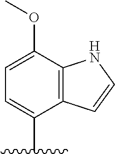 [SMILES: *c1ccc(OC)c2[nH]ccc12] | H |
| 1243 | 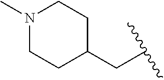 [SMILES: *CC1CCN(C)CC1] | NH | CH | N | CH₂ | NH | 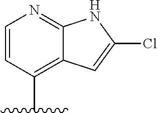 [SMILES: *c1ccnc2[nH]c(Cl)cc12] | H |
| 1244 | 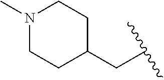 [SMILES: *CC1CCN(C)CC1] | NH | CH | N | CH₂ | NH | 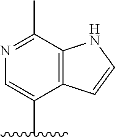 [SMILES: *c1cnc(C)c2[nH]ccc12] | H |
| 1245 | 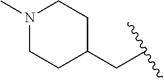 [SMILES: *CC1CCN(C)CC1] | NH | CH | N | CH₂ | NH | 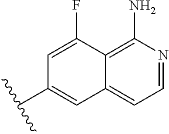 [SMILES: *c1cc(F)c2c(N)nccc2c1] | H |
| 1246 | 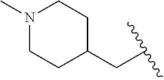 [SMILES: *CC1CCN(C)CC1] | NH | CH | N | CH₂ | NH | 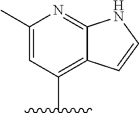 [SMILES: *c1cc(C)nc2[nH]ccc12] | H |
| 1247 | 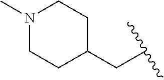 [SMILES: *CC1CCN(C)CC1] | NH | CH | N | CH₂ | NH | 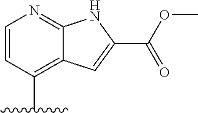 [SMILES: *c1ccnc2[nH]c(C(=O)OC)cc12] | H |
| 1248 | 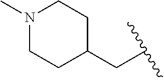 [SMILES: *CC1CCN(C)CC1] | NH | CH | N | CH₂ | NH | 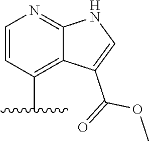 [SMILES: *c1ccnc2[nH]cc(C(=O)OC)c12] | H |
| 1249 | 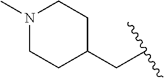 [SMILES: *CC1CCN(C)CC1] | O | CH | N | CH₂ | NH | 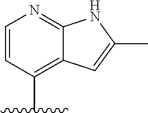 [SMILES: *c1ccnc2[nH]c(C)cc12] | H |
| 1250 | 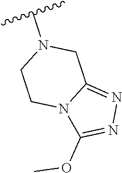 [SMILES: *N1CCn2c(nnc2OC)C1] | Absent | CH | N | CH₂ | NH | 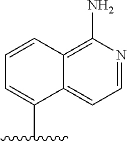 [SMILES: *c1cccc2c(N)nccc12] | H |
| 1251 | 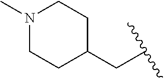 [SMILES: *CC1CCN(C)CC1] | NH | CH | N | CH₂ | NH | 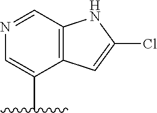 [SMILES: *c1cncc2[nH]c(Cl)cc12] | H |
| 1252 | 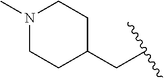 [SMILES: *CC1CCN(C)CC1] | NH | CH | N | CH₂ | NH | 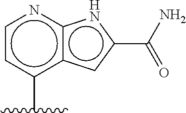 | H |
| 1253 | 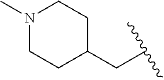 [SMILES: *CC1CCN(C)CC1] | NH | CH | N | CH₂ | NH | 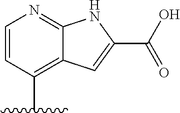 [SMILES: *c1ccnc2[nH]c(C(=O)O)cc12] | H |
| 1254 | 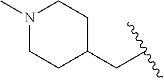 [SMILES: *CC1CCN(C)CC1] | NH | CH | N | CH₂ | NH | 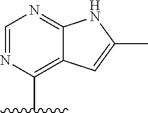 [SMILES: *c1ncnc2[nH]c(C)cc12] | H |
| 1255 | 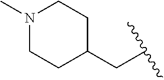 [SMILES: *CC1CCN(C)CC1] | NH | CH | N | CH₂ | NH | 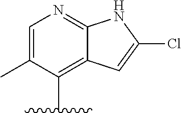 [SMILES: *c1c(C)cnc2[nH]c(Cl)cc12] | H |
| 1256 | 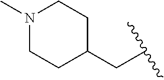 [SMILES: *CC1CCN(C)CC1] | NH | CH | N | CH₂ | NH | 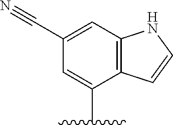 [SMILES: *c1cc(C#N)cc2[nH]ccc12] | H |
| 1257 | 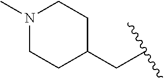 [SMILES: *CC1CCN(C)CC1] | NH | CH | N | CH₂ | NH | 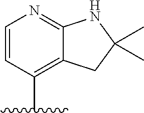 [SMILES: *c1ccnc2c1CC(C)(C)N2] | H |
| 1258 | 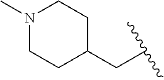 [SMILES: *CC1CCN(C)CC1] | NH | CH | N | CH₂ | NH | 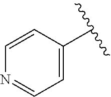 [SMILES: *c1ccncc1] | H |
| 1259 | 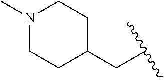 [SMILES: *CC1CCN(C)CC1] | NH | CH | N | CH₂ | NH | 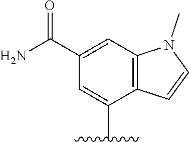 [SMILES: *c1cc(C(N)=O)cc2c1ccn2C] | H |
| 1260 | 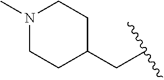 [SMILES: *CC1CCN(C)CC1] | NH | CH | N | CH₂ | NH | 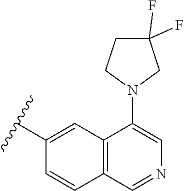 [SMILES: *c1ccc2cncc(N3CCC(F)(F)C3)c2c1] | H |
| 1261 | 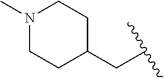 [SMILES: *CC1CCN(C)CC1] | NH | CH | N | CH₂ | NH | 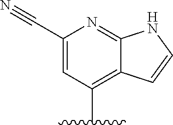 [SMILES: *c1cc(C#N)nc2[nH]ccc12] | H |
| 1262 | 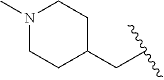 [SMILES: *CC1CCN(C)CC1] | NH | CH | N | CH₂ | NH | 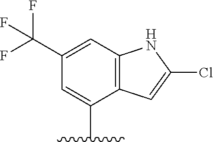 [SMILES: *c1cc(C(F)(F)F)cc2[nH]c(Cl)cc12] | H |
| 1263 | 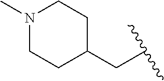 [SMILES: *CC1CCN(C)CC1] | NH | CH | N | CH₂ | NH | 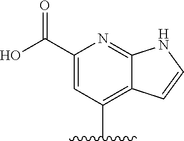 [SMILES: *c1cc(C(=O)O)nc2[nH]ccc12] | H |
| 1264 | 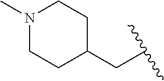 [SMILES: *CC1CCN(C)CC1] | NH | CH | N | CH₂ | NH | 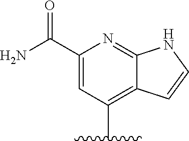 [SMILES: *c1cc(C(N)=O)nc2[nH]ccc12] | H |
| 1265 | 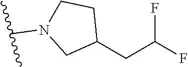 [SMILES: *N1CCC(CC(F)F)C1] | Absent | N | CH | CH₂ | NH | 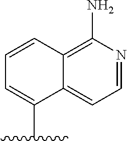 [SMILES: *c1cccc2c(N)nccc12] | H |
| 1266 | 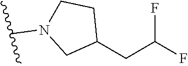 [SMILES: *N1CCC(CC(F)F)C1] | Absent | N | CH | CH₂ | NH | 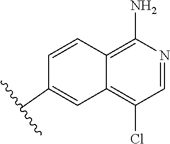 [SMILES: *c1ccc2c(N)ncc(Cl)c2c1] | H |
| 1267 | 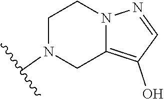 [SMILES: *N1CCn2ncc(O)c2C1] | Absent | N | CH | CH₂ | NH | 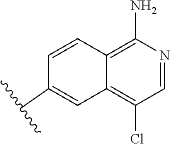 [SMILES: *c1ccc2c(N)ncc(Cl)c2c1] | H |
| 1268 | 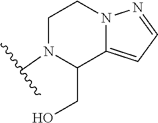 [SMILES: *N1CCn2nccc2C1CO] | Absent | N | CH | CH₂ | NH | 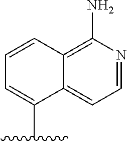 [SMILES: *c1cccc2c(N)nccc12] | H |
| 1269 | 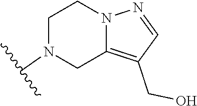 [SMILES: *N1CCn2ncc(CO)c2C1] | Absent | N | CH | CH₂ | NH | 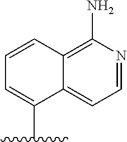 [SMILES: *c1cccc2c(N)nccc12] | H |
| 1270 | 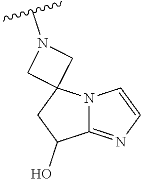 [SMILES: *N1CC2(CC(O)c3nccn32)C1] | Absent | N | CH | CH₂ | NH | 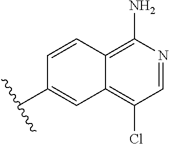 [SMILES: *c1ccc2c(N)ncc(Cl)c2c1] | H |
| 1271 | 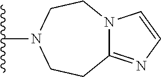 [SMILES: *N1CCc2nccn2CC1] | Absent | N | CH | CH₂ | NH | 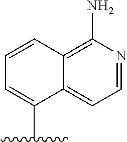 [SMILES: *c1cccc2c(N)nccc12] | H |
| 1272 | 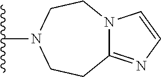 [SMILES: *N1CCc2nccn2CC1] | Absent | N | CH | CH₂ | NH | 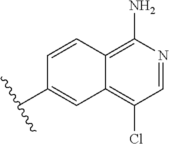 [SMILES: *c1ccc2c(N)ncc(Cl)c2c1] | H |
| 1273 | 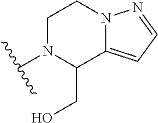 [SMILES: *N1CCn2nccc2C1CO] | Absent | N | CH | CH₂ | NH | 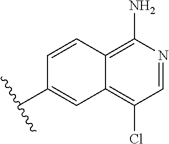 [SMILES: *c1ccc2c(N)ncc(Cl)c2c1] | H |
| 1274 | 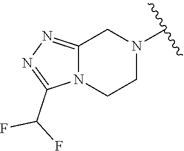 [SMILES: *N1CCn2c(nnc2C(F)F)C1] | Absent | N | CH | CH₂ | NH | 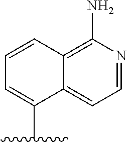 [SMILES: *c1cccc2c(N)nccc12] | H |
| 1275 | 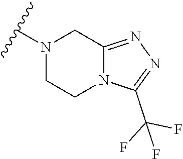 [SMILES: *N1CCn2c(nnc2C(F)(F)F)C1] | Absent | N | CH | CH₂ | NH | 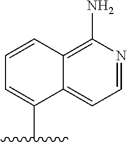 [SMILES: *c1cccc2c(N)nccc12] | H |
| 1276 | 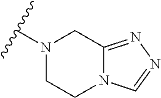 [SMILES: *N1CCn2cnnc2C1] | Absent | N | CH | CH₂ | NH | 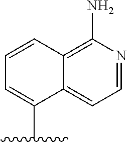 [SMILES: *c1cccc2c(N)nccc12] | H |
| 1277 | 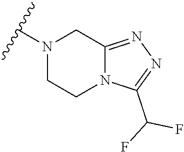 [SMILES: *N1CCn2c(nnc2C(F)F)C1] | Absent | N | CH | CH₂ | NH | 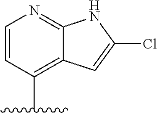 [SMILES: *c1ccnc2[nH]c(Cl)cc12] | H |
| 1278 | 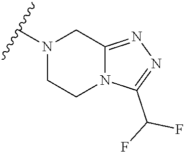 [SMILES: *N1CCn2c(nnc2C(F)F)C1] | Absent | N | CH | CH₂ | NH | 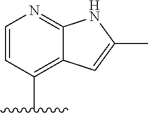 [SMILES: *c1ccnc2[nH]c(C)cc12] | H |
| 1279 | 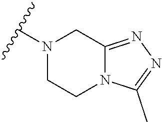 [SMILES: *N1CCn2c(C)nnc2C1] | Absent | N | CH | CH₂ | NH | 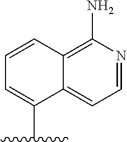 [SMILES: *c1cccc2c(N)nccc12] | H |
| 1280 | 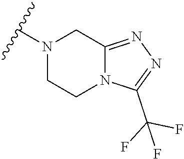 [SMILES: *N1CCn2c(nnc2C(F)(F)F)C1] | Absent | N | CH | CH₂ | NH | 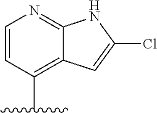 [SMILES: *c1ccnc2[nH]c(Cl)cc12] | H |
| 1281 | 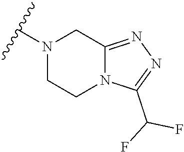 [SMILES: *N1CCn2c(nnc2C(F)F)C1] | Absent | N | CH | CH₂ | NH | 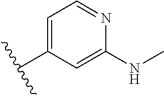 [SMILES: *c1ccnc(NC)c1] | H |
| 1282 | 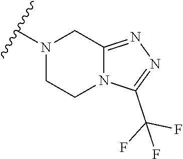 [SMILES: *N1CCn2c(nnc2C(F)(F)F)C1] | Absent | N | CH | CH₂ | NH | 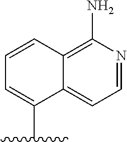 [SMILES: *c1cccc2c(N)nccc12] | CH₃ |
| 1283 | 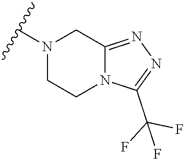 [SMILES: *N1CCn2c(nnc2C(F)(F)F)C1] | Absent | N | CH | CH₂ | NH | 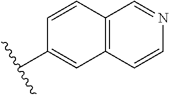 [SMILES: *c1ccc2cnccc2c1] | H |
| 1284 | 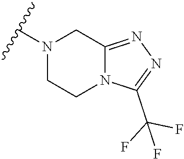 [SMILES: *N1CCn2c(nnc2C(F)(F)F)C1] | Absent | CH | N | CH₂ | NH | 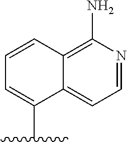 [SMILES: *c1cccc2c(N)nccc12] | CH₃ |
| 1285 | 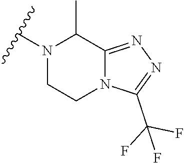 [SMILES: *N1CCn2c(nnc2C(F)(F)F)C1C] | Absent | N | CH | CH₂ | NH | 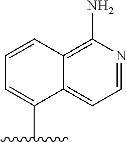 [SMILES: *c1cccc2c(N)nccc12] | H |
| 1286 | 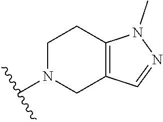 [SMILES: *N1CCc2c(cnn2C)C1] | Absent | N | CH | CH₂ | NH | 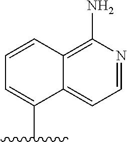 [SMILES: *c1cccc2c(N)nccc12] | H |
| 1287 | 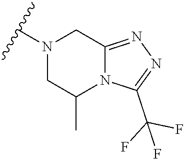 [SMILES: *N1Cc2nnc(C(F)(F)F)n2C(C)C1] | Absent | N | CH | CH₂ | NH | 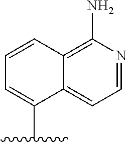 [SMILES: *c1cccc2c(N)nccc12] | H |
| 1288 | 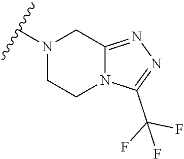 [SMILES: *N1CCn2c(nnc2C(F)(F)F)C1] | Absent | N | CH | CH₂ | NH | 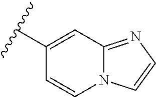 [SMILES: *c1ccn2ccnc2c1] | H |
| 1289 | 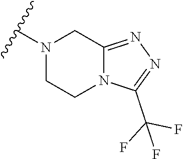 [SMILES: *N1CCn2c(nnc2C(F)(F)F)C1] | Absent | N | CH | CH₂ | NH | 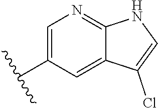 [SMILES: *c1cnc2[nH]cc(Cl)c2c1] | H |
| 1290 | 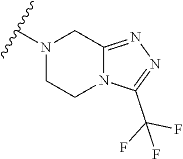 [SMILES: *N1CCn2c(nnc2C(F)(F)F)C1] | Absent | N | CH | CH₂ | NH | 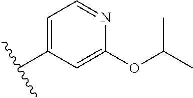 [SMILES: *c1ccnc(OC(C)C)c1] | H |
| 1291 | 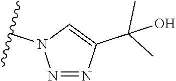 [SMILES: *n1cc(C(C)(C)O)nn1] | Absent | N | CH | CH₂ | NH | 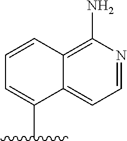 [SMILES: *c1cccc2c(N)nccc12] | H |
| 1292 | 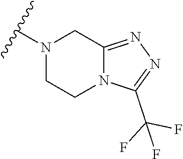 [SMILES: *N1CCn2c(nnc2C(F)(F)F)C1] | Absent | N | CH | CH₂ | NH | 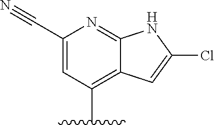 [SMILES: *c1cc(C#N)nc2[nH]c(Cl)cc12] | H |
| 1293 | 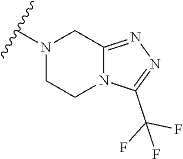 [SMILES: *N1CCn2c(nnc2C(F)(F)F)C1] | Absent | N | CH | CH₂ | NH | 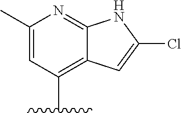 [SMILES: *c1cc(C)nc2[nH]c(Cl)cc12] | H |
| 1294 | 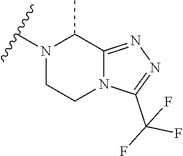 [SMILES: *N1CCn2c(nnc2C(F)(F)F)[C@H]1C] | Absent | N | CH | CH₂ | NH | 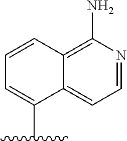 [SMILES: *c1cccc2c(N)nccc12] | H |
| 1295 | 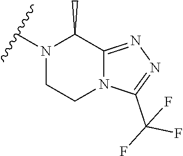 [SMILES: *N1CCn2c(nnc2C(F)(F)F)[C@@H]1C] | Absent | N | CH | CH₂ | NH | 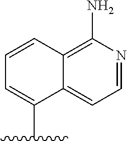 [SMILES: *c1cccc2c(N)nccc12] | H |
| 1296 | 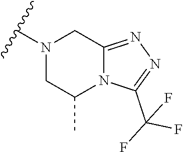 [SMILES: *N1Cc2nnc(C(F)(F)F)n2[C@H](C)C1] | Absent | N | CH | CH₂ | NH | 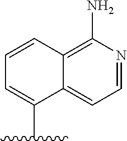 [SMILES: *c1cccc2c(N)nccc12] | H |
| 1297 | 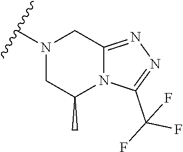 [SMILES: *N1Cc2nnc(C(F)(F)F)n2[C@@H](C)C1] | Absent | N | CH | CH₂ | NH | 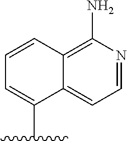 [SMILES: *c1cccc2c(N)nccc12] | H |
| 1298 | 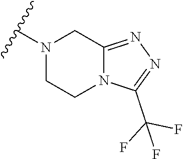 [SMILES: *N1CCn2c(nnc2C(F)(F)F)C1] | Absent | N | CH | CH₂ | NH | 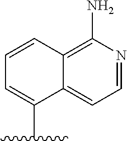 [SMILES: *c1cccc2c(N)nccc12] | CF₃ |
| 1299 | 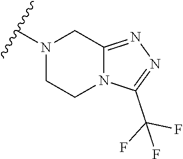 [SMILES: *N1CCn2c(nnc2C(F)(F)F)C1] | Absent | CH | N | CH₂ | NH | 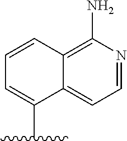 [SMILES: *c1cccc2c(N)nccc12] | Cl |
| 1300 | 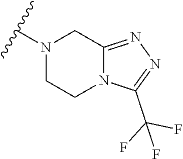 [SMILES: *N1CCn2c(nnc2C(F)(F)F)C1] | Absent | CH | CH | CH₂ | NH | 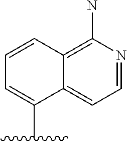 [SMILES: *c1cccc2c(N)nccc12] | H |
| 1301 | 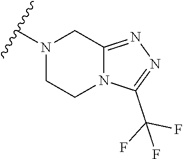 [SMILES: *N1CCn2c(nnc2C(F)(F)F)C1] | Absent | CH | N | CH₂ | NH | 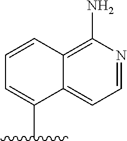 [SMILES: *c1cccc2c(N)nccc12] | CF₃ |
| 1302 | 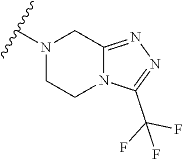 [SMILES: *N1CCn2c(nnc2C(F)(F)F)C1] | Absent | N | CCH3 | CH₂ | NH | 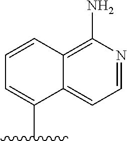 [SMILES: *c1cccc2c(N)nccc12] | H |
| 1303 | 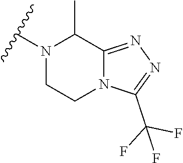 [SMILES: *N1CCn2c(nnc2C(F)(F)F)C1C] | Absent | CH | N | CH₂ | NH | 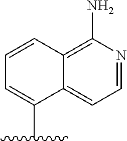 [SMILES: *c1cccc2c(N)nccc12] | CH₃ |
| 1304 | 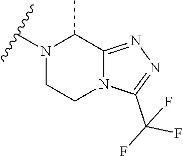 [SMILES: *N1CCn2c(nnc2C(F)(F)F)[C@H]1C] | Absent | CH | N | CH₂ | NH | 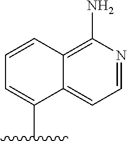 [SMILES: *c1cccc2c(N)nccc12] | CH₃ |
| 1305 | 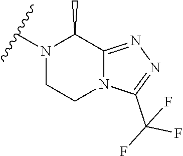 [SMILES: *N1CCn2c(nnc2C(F)(F)F)[C@@H]1C] | Absent | CH | N | CH₂ | NH | 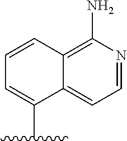 [SMILES: *c1cccc2c(N)nccc12] | CH₃ |
| 1306 | 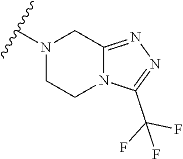 [SMILES: *N1CCn2c(nnc2C(F)(F)F)C1] | Absent | N | CCF₃ | CH₂ | NH | 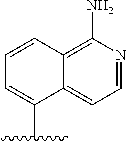 [SMILES: *c1cccc2c(N)nccc12] | H |
| 1307 | 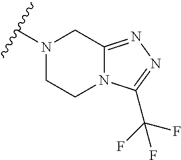 [SMILES: *N1CCn2c(nnc2C(F)(F)F)C1] | Absent | CH | N | CH₂ | NH | 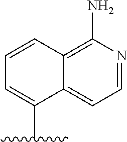 [SMILES: *c1cccc2c(N)nccc12] | 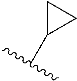 [SMILES: *C1CC1] |
| 1308 | 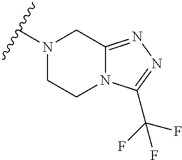 [SMILES: *N1CCn2c(nnc2C(F)(F)F)C1] | Absent | CH | N | CH₂ | NH | 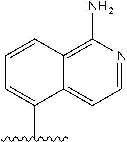 [SMILES: *c1cccc2c(N)nccc12] | iPr |
| 1309 | 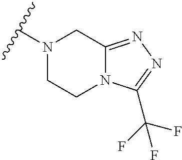 [SMILES: *N1CCn2c(nnc2C(F)(F)F)C1] | Absent | CH | N | CH₂ | NH | 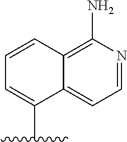 [SMILES: *c1cccc2c(N)nccc12] | Et |
| 1310 | 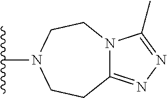 [SMILES: *N1CCc2nnc(C)n2CC1] | Absent | N | CH | CH₂ | NH | 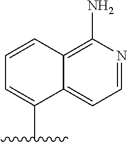 [SMILES: *c1cccc2c(N)nccc12] | H |
| 1311 | 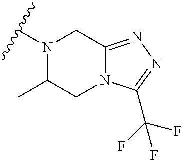 [SMILES: *N1Cc2nnc(C(F)(F)F)n2CC1C] | Absent | N | CH | CH₂ | NH | 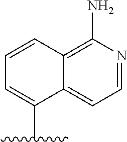 [SMILES: *c1cccc2c(N)nccc12] | H |
| 1312 | 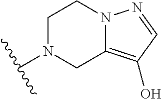 [SMILES: *N1CCn2ncc(O)c2C1] | Absent | N | CH | CH₂ | NH | 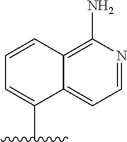 [SMILES: *c1cccc2c(N)nccc12] | H |
| 1313 | 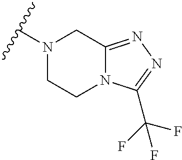 [SMILES: *N1CCn2c(nnc2C(F)(F)F)C1] | Absent | CH | N | CH₂ | NH | 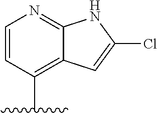 [SMILES: *c1ccnc2[nH]c(Cl)cc12] | CH₃ |
| 1314 | 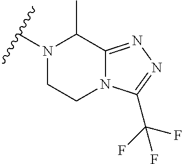 [SMILES: *N1CCn2c(nnc2C(F)(F)F)C1C] | Absent | CH | N | CH₂ | NH | 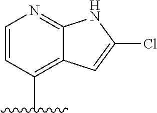 [SMILES: *c1ccnc2[nH]c(Cl)cc12] | CH₃ |
| 1315 | 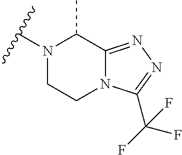 [SMILES: *N1CCn2c(nnc2C(F)(F)F)[C@H]1C] | Absent | CH | N | CH₂ | NH | 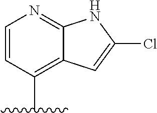 [SMILES: *c1ccnc2[nH]c(Cl)cc12] | CH₃ |
| 1316 | 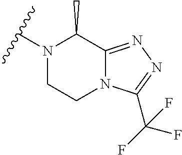 [SMILES: *N1CCn2c(nnc2C(F)(F)F)[C@@H]1C] | Absent | CH | N | CH₂ | NH | 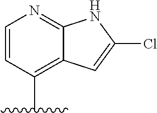 [SMILES: *c1ccnc2[nH]c(Cl)cc12] | CH₃ |
| 1317 | 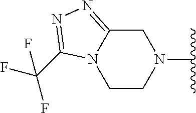 [SMILES: *N1CCn2c(nnc2C(F)(F)F)C1] | Absent | N | CH | CH₂ | NH | 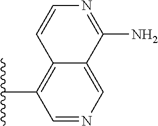 [SMILES: *c1cncc2c(N)nccc12] | H |
| 1318 | 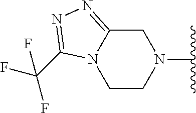 [SMILES: *N1CCn2c(nnc2C(F)(F)F)C1] | Absent | N | CH | CH₂ | NH | 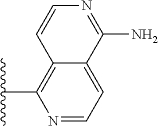 [SMILES: *c1nccc2c(N)nccc12] | H |
| 1319 | 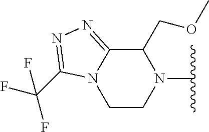 [SMILES: *N1CCn2c(nnc2C(F)(F)F)C1COC] | Absent | CH | N | CH₂ | NH | 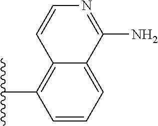 [SMILES: *c1cccc2c(N)nccc12] | CH₃ |
| 1320 | 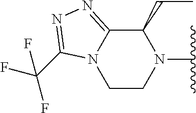 [SMILES: *N1CCn2c(nnc2C(F)(F)F)[C@H]1CC] | Absent | CH | N | CH₂ | NH | 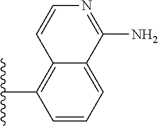 [SMILES: *c1cccc2c(N)nccc12] | CH₃ |
| 1321 | 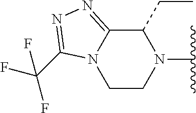 [SMILES: *N1CCn2c(nnc2C(F)(F)F)[C@@H]1CC] | Absent | CH | N | CH₂ | NH | 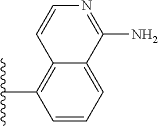 [SMILES: *c1cccc2c(N)nccc12] | CH₃ |
| 1322 | 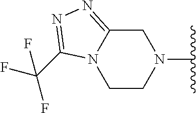 [SMILES: *N1CCn2c(nnc2C(F)(F)F)C1] | Absent | CH | N | CH₂ | NH | 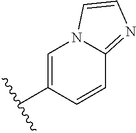 [SMILES: *c1ccc2nccn2c1] | CH₃ |
| 1323 | 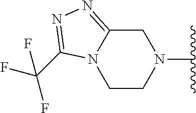 [SMILES: *N1CCn2c(nnc2C(F)(F)F)C1] | Absent | CH | N | CH₂ | NH | 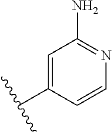 [SMILES: *c1ccnc(N)c1] | CH₃ |
| 1324 | 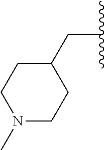 [SMILES: *CC1CCN(C)CC1] | O | CH | N | CH₂ | NH | 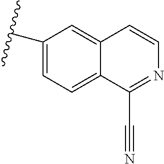 [SMILES: *c1ccc2c(C#N)nccc2c1] | H |
| 1325 | 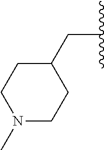 [SMILES: *CC1CCN(C)CC1] | O | CH | N | CH₂ | NH | 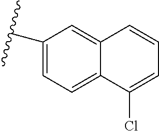 [SMILES: *c1ccc2c(Cl)cccc2c1] | H |
| 1326 | 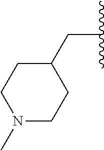 [SMILES: *CC1CCN(C)CC1] | O | CH | N | CH₂ | NH | 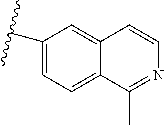 [SMILES: *c1ccc2c(C)nccc2c1] | H |
| 1327 | 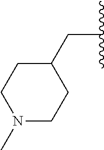 [SMILES: *CC1CCN(C)CC1] | O | CH | N | CH₂ | NH | 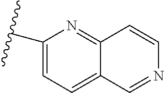 [SMILES: *c1ccc2cnccc2n1] | H |
| 1328 | 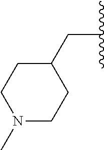 [SMILES: *CC1CCN(C)CC1] | O | CH | N | CH₂ | NH | 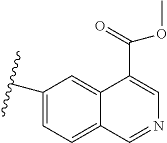 [SMILES: *c1ccc2cncc(C(=O)OC)c2c1] | H |
| 1329 | 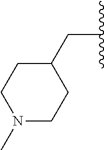 [SMILES: *CC1CCN(C)CC1] | O | CH | N | CH₂ | NH | 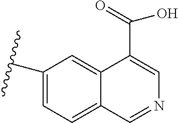 [SMILES: *c1ccc2cncc(C(=O)O)c2c1] | H |
| 1330 | 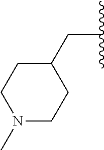 [SMILES: *CC1CCN(C)CC1] | O | CH | N | CH₂ | NH | 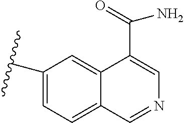 [SMILES: *c1ccc2cncc(C(N)=O)c2c1] | H |
| 1331 | 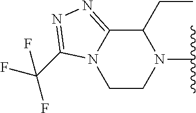 [SMILES: *N1CCn2c(nnc2C(F)(F)F)C1CC] | Absent | CH | N | CH₂ | NH | 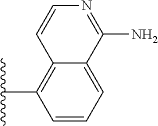 [SMILES: *c1cccc2c(N)nccc12] | CH₃ |
| 1332 | 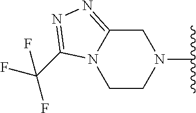 [SMILES: *N1CCn2c(nnc2C(F)(F)F)C1] | Absent | CH | N | CH₂ | NCH₃ | 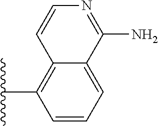 [SMILES: *c1cccc2c(N)nccc12] | H |
| 1333 | 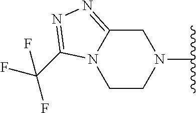 [SMILES: *N1CCn2c(nnc2C(F)(F)F)C1] | Absent | CH | N | CH₂ | NH | 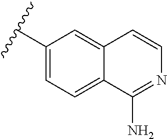 [SMILES: *c1ccc2c(N)nccc2c1] | CH₃ |
| 1334 | 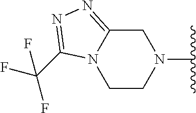 [SMILES: *N1CCn2c(nnc2C(F)(F)F)C1] | Absent | CH | N | CH₂ | NH | 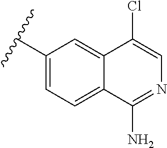 [SMILES: *c1ccc2c(N)ncc(Cl)c2c1] | CH₃ |
| 1335 | 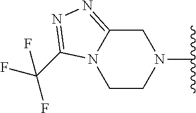 [SMILES: *N1CCn2c(nnc2C(F)(F)F)C1] | Absent | N | CCH₂OH | CH₂ | NH | 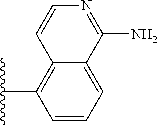 [SMILES: *c1cccc2c(N)nccc12] | H |
| 1336 | 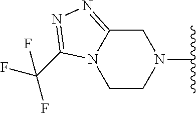 [SMILES: *N1CCn2c(nnc2C(F)(F)F)C1] | Absent | N | CH | CH₂ | NH | 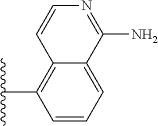 [SMILES: *c1cccc2c(N)nccc12] | CH₂CH₃ |
| 1337 | 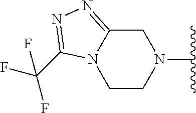 [SMILES: *N1CCn2c(nnc2C(F)(F)F)C1] | Absent | CH | N | SO₂ | NH | 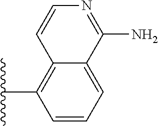 [SMILES: *c1cccc2c(N)nccc12] | H |
| 1338 | 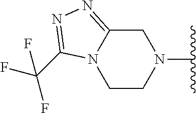 [SMILES: *N1CCn2c(nnc2C(F)(F)F)C1] | Absent | CH | N | CH₂ | NH | 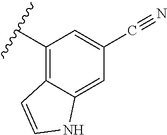 [SMILES: *c1cc(C#N)cc2[nH]ccc12] | H |
| 1339 | 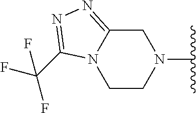 [SMILES: *N1CCn2c(nnc2C(F)(F)F)C1] | Absent | CH | N | CH₂ | NH | 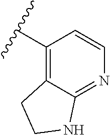 [SMILES: *c1ccnc2c1CCN2] | CH₃ |
| 1341 | 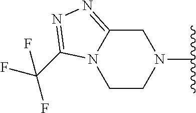 [SMILES: *N1CCn2c(nnc2C(F)(F)F)C1] | Absent | CH | N | CH₂ | NH | 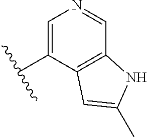 [SMILES: *c1cncc2[nH]c(C)cc12] | CH₃ |
| 1342 | 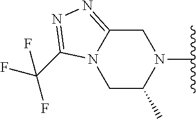 [SMILES: *N1Cc2nnc(C(F)(F)F)n2C[C@H]1C] | Absent | CH | N | CH₂ | NH | 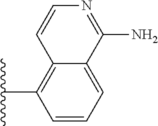 [SMILES: *c1cccc2c(N)nccc12] | CH₃ |
| 1343 | 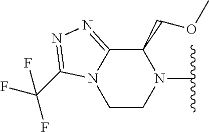 [SMILES: *N1CCn2c(nnc2C(F)(F)F)[C@H]1COC] | Absent | CH | N | CH₂ | NH | 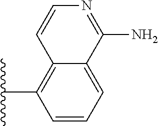 [SMILES: *c1cccc2c(N)nccc12] | CH₃ |
| 1344 | 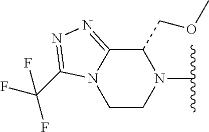 [SMILES: *N1CCn2c(nnc2C(F)(F)F)[C@@H]1COC] | Absent | CH | N | CH₂ | NH | 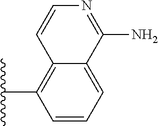 [SMILES: *c1cccc2c(N)nccc12] | CH₃ |

TABLE 2a

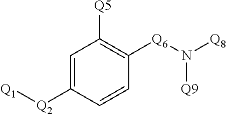

| Example | | | | | | |
|---|---|---|---|---|---|---|
| Number | Q₁ | Q₂ | Q₆ | Q₉ | Q₈ | Q₅ |
| 2177 | 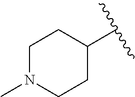 [SMILES: *C1CCN(C)CC1] | CH₂CH₂ | CH₂ | H | 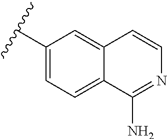 [SMILES: *c1ccc2c(N)nccc2c1] | H |
| 2178 | 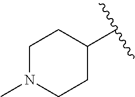 [SMILES: *C1CCN(C)CC1] | CH₂CH₂ | CH₂ | H | 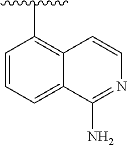 [SMILES: *c1cccc2c(N)nccc12] | H |
| 2179 | 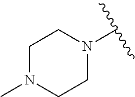 [SMILES: *N1CCN(C)CC1] | COCH₂ | CH₂ | H | 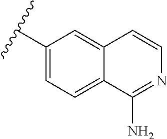 [SMILES: *c1ccc2c(N)nccc2c1] | H |
| 2180 | 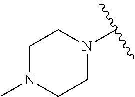 [SMILES: *N1CCN(C)CC1] | CH₂CH₂ | CH₂ | H | 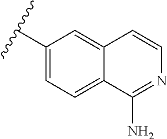 [SMILES: *c1ccc2c(N)nccc2c1] | H |
| 2181 | 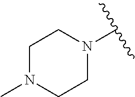 [SMILES: *N1CCN(C)CC1] | CH₂CH₂ | CH₂ | H | 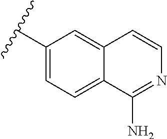 [SMILES: *c1ccc2c(N)nccc2c1] | F |
| 2182 | 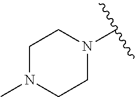 [SMILES: *N1CCN(C)CC1] | CH₂CH₂ | CH₂ | H | 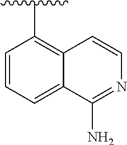 [SMILES: *c1cccc2c(N)nccc12] | F |
| 2183 | 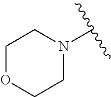 [SMILES: *N1CCOCC1] | CH₂CH₂ | CH₂ | H | 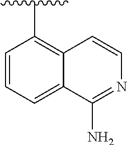 [SMILES: *c1cccc2c(N)nccc12] | F |
| 2184 | 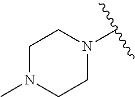 [SMILES: *N1CCN(C)CC1] | CH₂CH₂ | CH₂ | H | 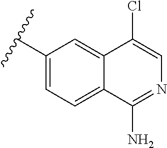 [SMILES: *c1ccc2c(N)ncc(Cl)c2c1] | F |
| 2185 | 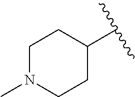 [SMILES: *C1CCN(C)CC1] | OCH₂ | CH₂ | H | 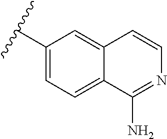 [SMILES: *c1ccc2c(N)nccc2c1] | H |
| 2186 | 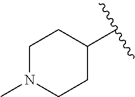 [SMILES: *C1CCN(C)CC1] | OCH₂ | CH₂ | H | 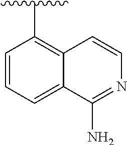 [SMILES: *c1cccc2c(N)nccc12] | H |
| 2187 | 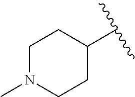 [SMILES: *C1CCN(C)CC1] | OCH₂ | CH₂ | H | 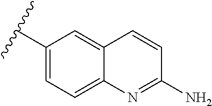 [SMILES: *c1ccc2nc(N)ccc2c1] | H |
| 2188 | 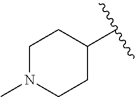 [SMILES: *C1CCN(C)CC1] | OCH₂ | CH₂ | H | 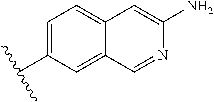 [SMILES: *c1ccc2cc(N)ncc2c1] | H |
| 2189 | 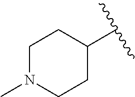 [SMILES: *C1CCN(C)CC1] | OCH₂ | CH₂ | H | 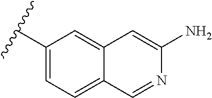 [SMILES: *c1ccc2cnc(N)cc2c1] | H |
| 2190 | 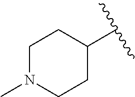 [SMILES: *C1CCN(C)CC1] | OCH₂ | CH₂ | CH₃ | 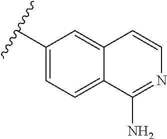 [SMILES: *c1ccc2c(N)nccc2c1] | H |
| 2191 | 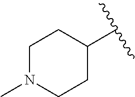 [SMILES: *C1CCN(C)CC1] | OCH₂ | CH₂ | H | 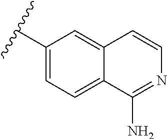 [SMILES: *c1ccc2c(N)nccc2c1] | F |
| 2192 | 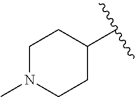 [SMILES: *C1CCN(C)CC1] | OCH₂ | CH₂ | H | 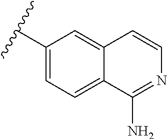 [SMILES: *c1ccc2c(N)nccc2c1] | Cl |
| 2193 | 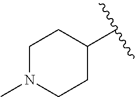 [SMILES: *C1CCN(C)CC1] | OCH₂ | CH₂ | H | 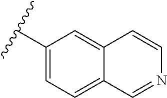 [SMILES: *c1ccc2cnccc2c1] | H |
| 2194 | 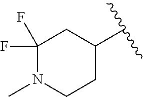 [SMILES: *C1CCN(C)C(F)(F)C1] | OCH₂ | CH₂ | H | 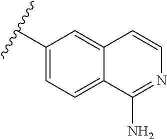 [SMILES: *c1ccc2c(N)nccc2c1] | H |
| 2195 | 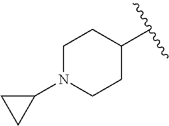 [SMILES: *C1CCN(C2CC2)CC1] | OCH₂ | CH₂ | H | 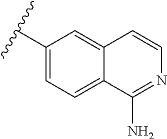 [SMILES: *c1ccc2c(N)nccc2c1] | H |
| 2196 | 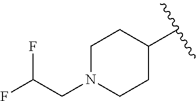 [SMILES: *C1CCN(CC(F)F)CC1] | OCH₂ | CH₂ | H | 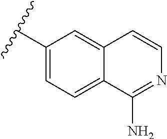 [SMILES: *c1ccc2c(N)nccc2c1] | H |
| 2197 | 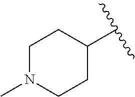 [SMILES: *C1CCN(C)CC1] | OCH₂ | CH₂ | H | 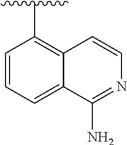 [SMILES: *c1cccc2c(N)nccc12] | F |
| 2198 | 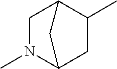 | OCH₂ | CH₂ | H | 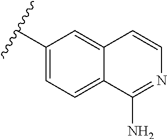 [SMILES: *c1ccc2c(N)nccc2c1] | F |
| 2199 | 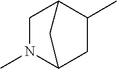 | OCH₂ | CH₂ | H | 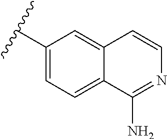 [SMILES: *c1ccc2c(N)nccc2c1] | F |
| 2200 | 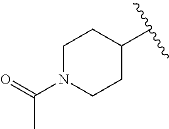 [SMILES: *C1CCN(C(C)=O)CC1] | OCH₂ | CH₂ | H | 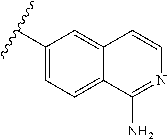 [SMILES: *c1ccc2c(N)nccc2c1] | F |
| 2201 | 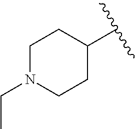 [SMILES: *C1CCN(CC)CC1] | OCH₂ | CH₂ | H | 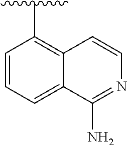 [SMILES: *c1cccc2c(N)nccc12] | F |
| 2202 | 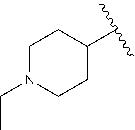 [SMILES: *C1CCN(CC)CC1] | OCH₂ | CH₂ | H | 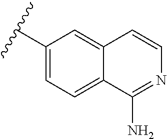 [SMILES: *c1ccc2c(N)nccc2c1] | F |
| 2203 | 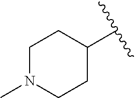 [SMILES: *C1CCN(C)CC1] | OCH₂ | CH₂ | CH₃ | 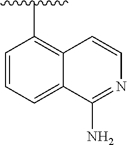 [SMILES: *c1cccc2c(N)nccc12] | Cl |
| 2204 | 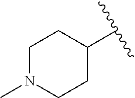 [SMILES: *C1CCN(C)CC1] | OCH₂ | CH₂ | CH₃ | 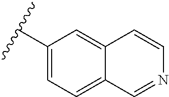 [SMILES: *c1ccc2cnccc2c1] | H |
| 2205 | 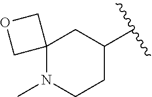 [SMILES: *C1CCN(C)C2(COC2)C1] | OCH₂ | CH₂ | H | 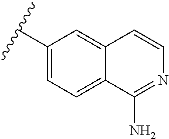 [SMILES: *c1ccc2c(N)nccc2c1] | F |
| 2206 | 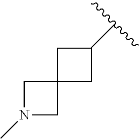 [SMILES: *C1CC2(C1)CN(C)C2] | OCH₂ | CH₂ | H | 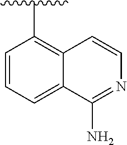 [SMILES: *c1cccc2c(N)nccc12] | F |
| 2207 | 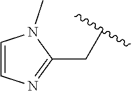 [SMILES: *Cc1nccn1C] | OCH₂ | CH₂ | H | 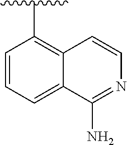 [SMILES: *c1cccc2c(N)nccc12] | F |
| 2208 | 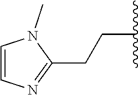 [SMILES: *CCc1nccn1C] | OCH₂ | CH₂ | H | 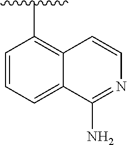 [SMILES: *c1cccc2c(N)nccc12] | F |
| 2209 | 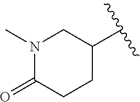 [SMILES: *C1CCC(=O)N(C)C1] | OCH₂ | CH₂ | H | 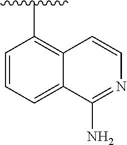 [SMILES: *c1cccc2c(N)nccc12] | F |
| 2210 | 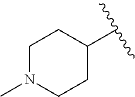 [SMILES: *C1CCN(C)CC1] | OCH₂ | SO₂ | H | 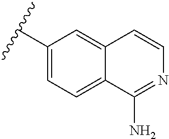 [SMILES: *c1ccc2c(N)nccc2c1] | H |
| 2211 | 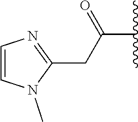 [SMILES: *C(=O)Cc1nccn1C] | NHCH₂ | CH₂ | H | 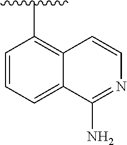 [SMILES: *c1cccc2c(N)nccc12] | F |
| 2212 | 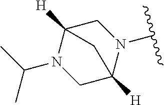 [SMILES: *N1C[C@@H]2C[C@H]1CN2C(C)C] | CH₂CH₂ | CH₂ | H | 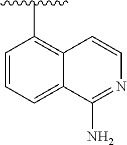 [SMILES: *c1cccc2c(N)nccc12] | F |
| 2213 | 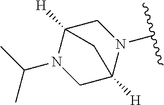 [SMILES: *N1C[C@H]2C[C@@H]1CN2C(C)C] | CH₂CH₂ | CH₂ | H | 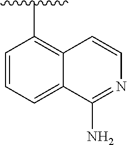 [SMILES: *c1cccc2c(N)nccc12] | F |
| 2214 | 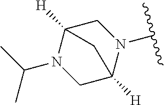 [SMILES: *N1C[C@H]2C[C@@H]1CN2C(C)C] | CH₂CH₂ | CH₂ | H | 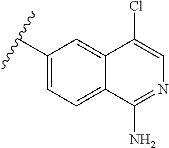 [SMILES: *c1ccc2c(N)ncc(Cl)c2c1] | F |
| 2215 | 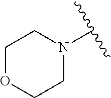 [SMILES: *N1CCOCC1] | CH₂CH₂ | CH₂ | H | 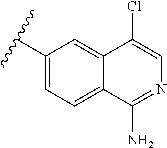 [SMILES: *c1ccc2c(N)ncc(Cl)c2c1] | F |
| 2216 | 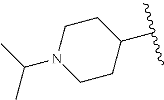 [SMILES: *C1CCN(C(C)C)CC1] | OCH₂ | CH₂ | H | 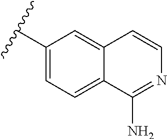 [SMILES: *c1ccc2c(N)nccc2c1] | F |
| 2217 | 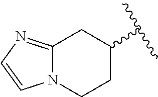 | OCH₂ | CH₂ | H | 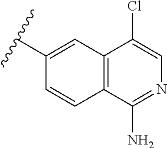 [SMILES: *c1ccc2c(N)ncc(Cl)c2c1] | CH₂OH |
| 2218 | 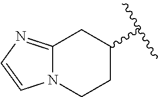 | OCH₂ | CH₂ | H | 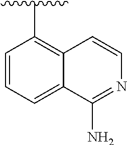 [SMILES: *c1cccc2c(N)nccc12] | CH₂OH |
| 2219 | 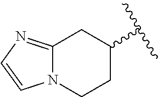 | OCH₂ | CH₂ | H | 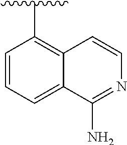 [SMILES: *c1cccc2c(N)nccc12] | F |
| 2220 | 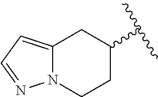 | OCH₂ | CH₂ | H | 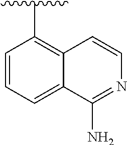 [SMILES: *c1cccc2c(N)nccc12] | F |
| 2221 | 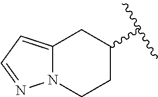 | OCH₂ | CH₂ | H | 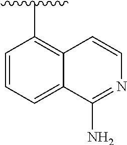 [SMILES: *c1cccc2c(N)nccc12] | CH₂OH |
| 2222 | 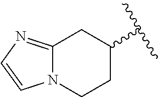 | OCH₂ | CH₂ | H | 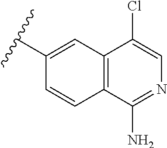 [SMILES: *c1ccc2c(N)ncc(Cl)c2c1] | F |
| 2223 | 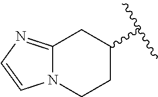 | OCH₂ | CH₂ | H | 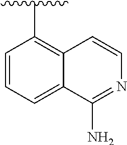 [SMILES: *c1cccc2c(N)nccc12] | F |
| 2224 | 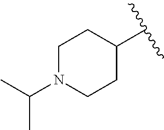 [SMILES: *C1CCN(C(C)C)CC1] | OCH₂ | CH₂ | H | 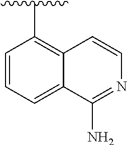 [SMILES: *c1cccc2c(N)nccc12] | Cl |
| 2225 | 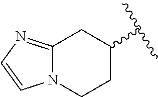 | OCH₂ | CH₂ | H | 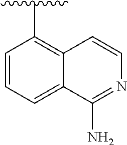 [SMILES: *c1cccc2c(N)nccc12] | Cl |
| 2226 | 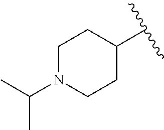 [SMILES: *C1CCN(C(C)C)CC1] | OCH₂ | CH₂ | H | 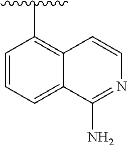 [SMILES: *c1cccc2c(N)nccc12] | F |
| 2227 | 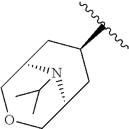 [SMILES: *[C@H]1C[C@H]2COC[C@@H](C1)N2C(C)C] | OCH₂ | CH₂ | H | 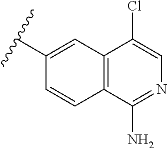 [SMILES: *c1ccc2c(N)ncc(Cl)c2c1] | F |
| 2228 | 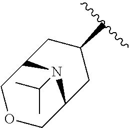 | OCH₂ | CH₂ | H | 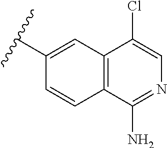 [SMILES: *c1ccc2c(N)ncc(Cl)c2c1] | F |
| 2229 | 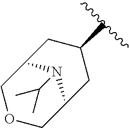 [SMILES: *[C@H]1C[C@H]2COC[C@@H](C1)N2C(C)C] | OCH₂ | CH₂ | H | 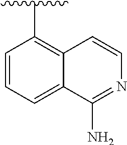 [SMILES: *c1cccc2c(N)nccc12] | F |
| 2230 | 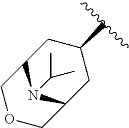 | OCH₂ | CH₂ | H | 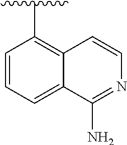 [SMILES: *c1cccc2c(N)nccc12] | F |
| 2231 | 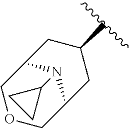 [SMILES: *[C@H]1C[C@H]2COC[C@@H](C1)N2C1CC1] | OCH₂ | CH₂ | H | 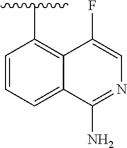 [SMILES: *c1cccc2c(N)ncc(F)c12] | F |
| 2232 | 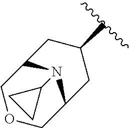 | OCH₂ | CH₂ | H | 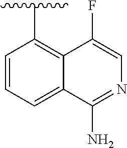 [SMILES: *c1cccc2c(N)ncc(F)c12] | F |
| 2233 | 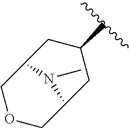 [SMILES: *[C@H]1C[C@H]2COC[C@@H](C1)N2C] | OCH₂ | CH₂ | H | 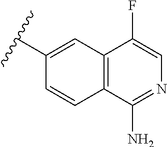 [SMILES: *c1ccc2c(N)ncc(F)c2c1] | F |
| 2234 | 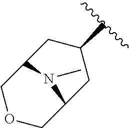 | OCH₂ | CH₂ | H | 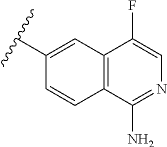 [SMILES: *c1ccc2c(N)ncc(F)c2c1] | F |
| 2235 | 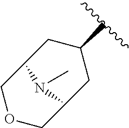 [SMILES: *[C@H]1C[C@H]2COC[C@@H](C1)N2C] | OCH₂ | CH₂ | H | 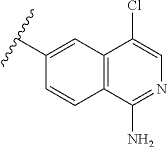 [SMILES: *c1ccc2c(N)ncc(Cl)c2c1] | F |
| 2236 | 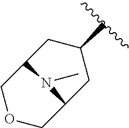 | OCH₂ | CH₂ | H | 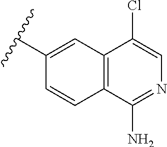 [SMILES: *c1ccc2c(N)ncc(Cl)c2c1] | F |
| 2237 | 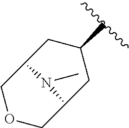 [SMILES: *[C@H]1C[C@H]2COC[C@@H](C1)N2C] | OCH₂ | CH₂ | H | 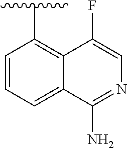 [SMILES: *c1cccc2c(N)ncc(F)c12] | F |
| 2238 | 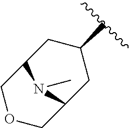 | OCH₂ | CH₂ | H | 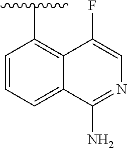 [SMILES: *c1cccc2c(N)ncc(F)c12] | F |
| 2239 | 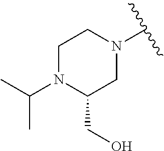 [SMILES: *N1CCN(C(C)C)[C@@H](CO)C1] | CH₂CH₂ | CH₂ | H | 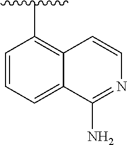 [SMILES: *c1cccc2c(N)nccc12] | F |
| 2240 | 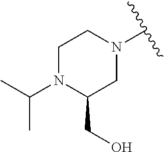 [SMILES: *N1CCN(C(C)C)[C@H](CO)C1] | CH₂CH₂ | CH₂ | H | 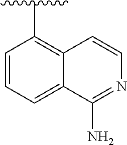 [SMILES: *c1cccc2c(N)nccc12] | F |
| 2241 | 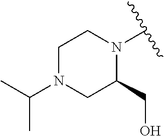 [SMILES: *N1CCN(C(C)C)C[C@@H]1CO] | CH₂CH₂ | CH₂ | H | 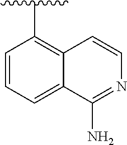 [SMILES: *c1cccc2c(N)nccc12] | F |
| 2242 | 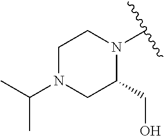 [SMILES: *N1CCN(C(C)C)C[C@H]1CO] | CH₂CH₂ | CH₂ | H | 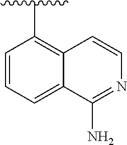 [SMILES: *c1cccc2c(N)nccc12] | F |
| 2243 | 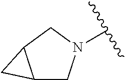 [SMILES: *N1CC2CC2C1] | CH₂CH₂ | CH₂ | H | 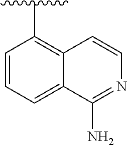 [SMILES: *c1cccc2c(N)nccc12] | F |
| 2244 | 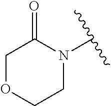 [SMILES: *N1CCOCC1=O] | CH₂CH₂ | CH₂ | H | 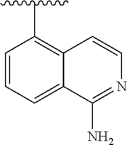 [SMILES: *c1cccc2c(N)nccc12] | F |
| 2245 | 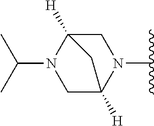 [SMILES: *N1C[C@H]2C[C@@H]1CN2C(C)C] | CH₂CH₂ | CH₂ | H | 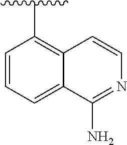 [SMILES: *c1cccc2c(N)nccc12] | CN |
| 2246 | 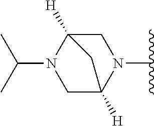 [SMILES: *N1C[C@H]2C[C@@H]1CN2C(C)C] | CH₂CH₂ | CH₂ | H | 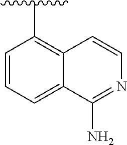 [SMILES: *c1cccc2c(N)nccc12] | CH₂OH |
| 2247 | 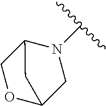 [SMILES: *N1CC2CC1CO2] | CH₂CH₂ | CH₂ | H | 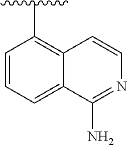 [SMILES: *c1cccc2c(N)nccc12] | CH₂OH |
| 2248 | 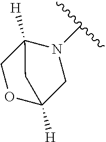 [SMILES: *N1C[C@@H]2C[C@H]1CO2] | CH₂CH₂ | CH₂ | H | 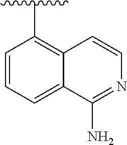 [SMILES: *c1cccc2c(N)nccc12] | F |
| 2249 | 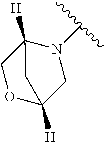 [SMILES: *N1C[C@H]2C[C@@H]1CO2] | CH₂CH₂ | CH₂ | H | 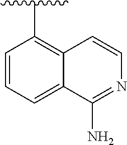 [SMILES: *c1cccc2c(N)nccc12] | F |
| 2250 | 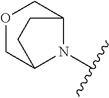 [SMILES: *N1C2CCC1COC2] | CH₂CH₂ | CH₂ | H | 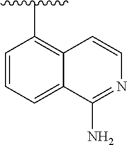 [SMILES: *c1cccc2c(N)nccc12] | F |
| 2251 | 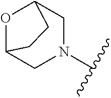 [SMILES: *N1CC2CCC(C1)O2] | CH₂CH₂ | CH₂ | H | 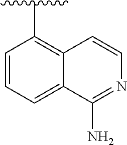 [SMILES: *c1cccc2c(N)nccc12] | F |
| 2252 | 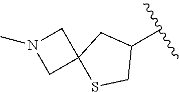 [SMILES: *C1CSC2(C1)CN(C)C2] | OCH₂ | CH₂ | H | 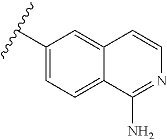 [SMILES: *c1ccc2c(N)nccc2c1] | F |

TABLE 2b

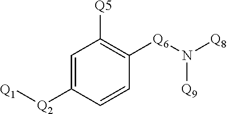

| Example | | | | | | |
|---|---|---|---|---|---|---|
| Number | Q₁ | Q₂ | Q₆ | Q₉ | Q₈ | Q₅ |
| 2253 | 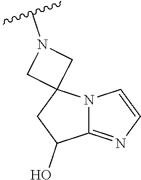 [SMILES: *N1CC2(CC(O)c3nccn32)C1] | Absent | CH₂ | H | 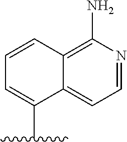 [SMILES: *c1cccc2c(N)nccc12] | H |
| 2254 | 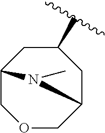 [SMILES: *[C@@H]1C[C@H]2COC[C@@H](C1)N2C] | OCH₂ | CH₂ | H | 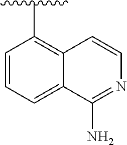 [SMILES: *c1cccc2c(N)nccc12] | F |
| 2255 | 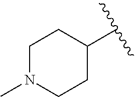 [SMILES: *C1CCN(C)CC1] | OCH₂ | CH₂ | H | 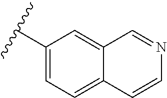 [SMILES: *c1ccc2ccncc2c1] | H |
| 2256 | 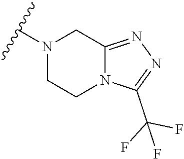 [SMILES: *N1CCn2c(nnc2C(F)(F)F)C1] | Absent | CH₂ | H | 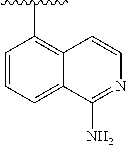 [SMILES: *c1cccc2c(N)nccc12] | F |
| 2257 | 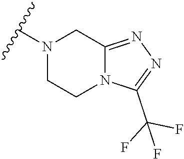 [SMILES: *N1CCn2c(nnc2C(F)(F)F)C1] | Absent | CH₂ | H | 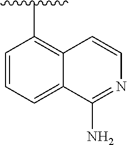 [SMILES: *c1cccc2c(N)nccc12] | CH₃ |

TABLE 3

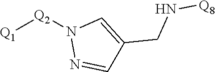

| Example Number | Q₁ | Q₂ | Q₈ |
|---|---|---|---|
| 3253 | 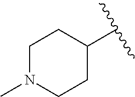 [SMILES: *C1CCN(C)CC1] | CH₂ | 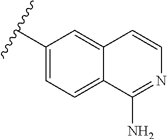 [SMILES: *c1ccc2c(N)nccc2c1] |
| 3254 | 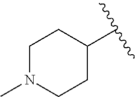 [SMILES: *C1CCN(C)CC1] | CH₂CH₂ | 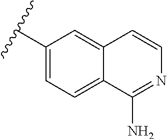 [SMILES: *c1ccc2c(N)nccc2c1] |
| 3255 | 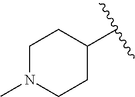 [SMILES: *C1CCN(C)CC1] | CH₂CH₂ | 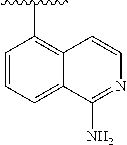 [SMILES: *c1cccc2c(N)nccc12] |
| 3256 | 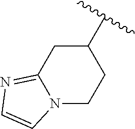 [SMILES: *C1CCn2ccnc2C1] | CH₂CH₂ | 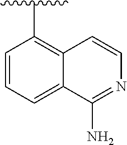 [SMILES: *c1cccc2c(N)nccc12] |
| 3257 | 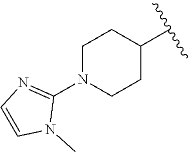 [SMILES: *C1CCN(c2nccn2C)CC1] | absent | 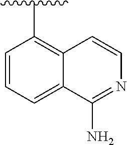 [SMILES: *c1cccc2c(N)nccc12] |
| 3258 | 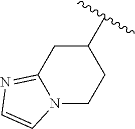 [SMILES: *C1CCn2ccnc2C1] | CH₂ | 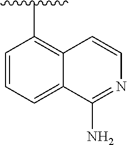 [SMILES: *c1cccc2c(N)nccc12] |

TABLE 4a

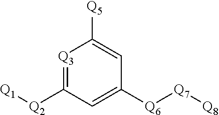

| Example | | | | | | | |
|---|---|---|---|---|---|---|---|
| Number | Q₁ | Q₂ | Q₃ | Q₆ | Q₇ | Q₈ | Q₅ |
| 4259 | 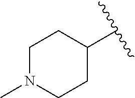 [SMILES: *C1CCN(C)CC1] | O | N | CH₂ | NH | 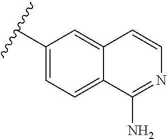 [SMILES: *c1ccc2c(N)nccc2c1] | H |
| 4260 | 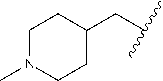 [SMILES: *CC1CCN(C)CC1] | O | N | CH₂ | NH | 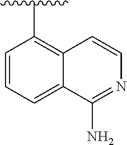 [SMILES: *c1cccc2c(N)nccc12] | H |
| 4261 | 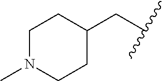 [SMILES: *CC1CCN(C)CC1] | O | N | CH₂ | NH | 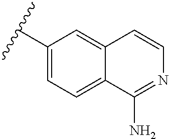 [SMILES: *c1ccc2c(N)nccc2c1] | H |
| 4262 | 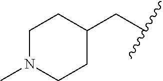 [SMILES: *CC1CCN(C)CC1] | O | N | CH₂ | NH | 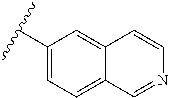 [SMILES: *c1ccc2cnccc2c1] | H |
| 4263 | 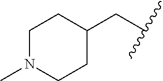 [SMILES: *CC1CCN(C)CC1] | O | N | CH₂ | NH | 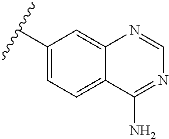 [SMILES: *c1ccc2c(N)ncnc2c1] | H |
| 4264 | 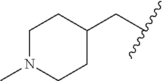 [SMILES: *CC1CCN(C)CC1] | O | N | CH₂ | NH | 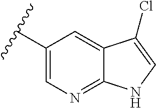 [SMILES: *c1cnc2[nH]cc(Cl)c2c1] | H |
| 4265 | 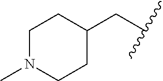 [SMILES: *CC1CCN(C)CC1] | O | N | CH₂ | NH | 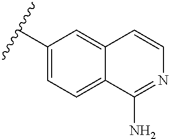 [SMILES: *c1ccc2c(N)nccc2c1] | CF₃ |
| 4266 | 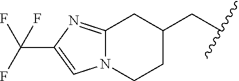 [SMILES: *CC1CCn2cc(C(F)(F)F)nc2C1] | O | N | CH₂ | NH | 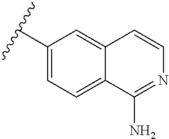 [SMILES: *c1ccc2c(N)nccc2c1] | H |
| 4267 | 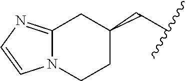 [SMILES: *C[C@H]1CCn2ccnc2C1] | O | N | CH₂ | NH | 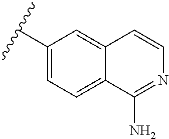 [SMILES: *c1ccc2c(N)nccc2c1] | H |
| 4268 | 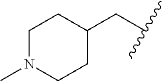 [SMILES: *CC1CCN(C)CC1] | O | N | CH₂ | NH | 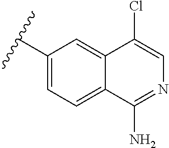 [SMILES: *c1ccc2c(N)ncc(Cl)c2c1] | H |
| 4269 | 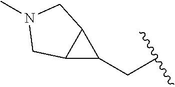 [SMILES: *CC1C2CN(C)CC12] | O | N | CH₂ | NH | 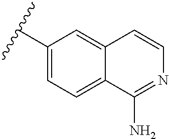 [SMILES: *c1ccc2c(N)nccc2c1] | H |
| 4270 | 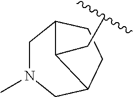 [SMILES: *CC1C2CCC1CN(C)C2] | O | N | CH₂ | NH | 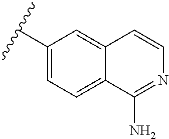 [SMILES: *c1ccc2c(N)nccc2c1] | H |
| 4271 | 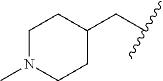 [SMILES: *CC1CCN(C)CC1] | O | N | CH₂ | NH | 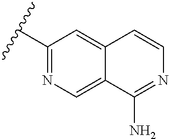 [SMILES: *c1cc2ccnc(N)c2cn1] | H |
| 4272 | 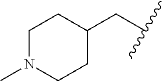 [SMILES: *CC1CCN(C)CC1] | O | N | CH₂ | NH | 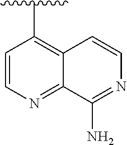 [SMILES: *c1ccnc2c(N)nccc12] | H |
| 4273 | 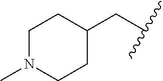 [SMILES: *CC1CCN(C)CC1] | O | N | CH₂ | NH | 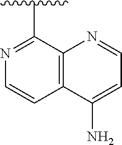 [SMILES: *c1nccc2c(N)ccnc12] | H |
| 4274 | 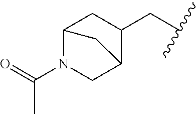 [SMILES: *CC1CC2CC1CN2C(C)=O] | O | N | CH₂ | NH | 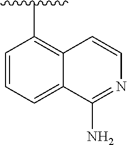 [SMILES: *c1cccc2c(N)nccc12] | H |
| 4275 | 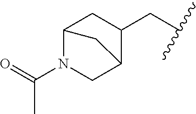 [SMILES: *CC1CC2CC1CN2C(C)=O] | O | N | CH₂ | NH | 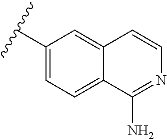 [SMILES: *c1ccc2c(N)nccc2c1] | H |
| 4276 | 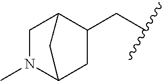 [SMILES: *CC1CC2CC1CN2C] | O | N | CH₂ | NH | 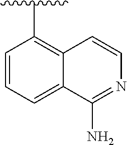 [SMILES: *c1cccc2c(N)nccc12] | H |
| 4277 | 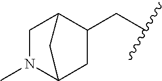 [SMILES: *CC1CC2CC1CN2C] | O | N | CH₂ | NH | 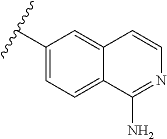 [SMILES: *c1ccc2c(N)nccc2c1] | H |
| 4278 | 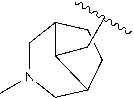 [SMILES: *CC1C2CCC1CN(C)C2] | O | N | CH₂ | NH | 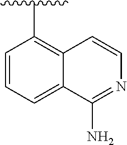 [SMILES: *c1cccc2c(N)nccc12] | H |
| 4279 | 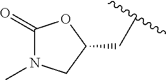 [SMILES: *C[C@@H]1CN(C)C(=O)O1] | O | N | CH₂ | NH | 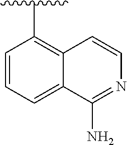 [SMILES: *c1cccc2c(N)nccc12] | H |
| 4280 | 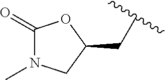 [SMILES: *C[C@H]1CN(C)C(=O)O1] | O | N | CH₂ | NH | 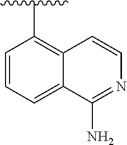 [SMILES: *c1cccc2c(N)nccc12] | H |
| 4281 | 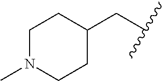 [SMILES: *CC1CCN(C)CC1] | O | N | CH₂ | NH | 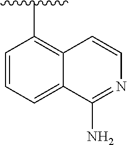 [SMILES: *c1cccc2c(N)nccc12] | CF₃ |
| 4282 | 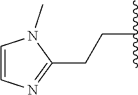 [SMILES: *CCc1nccn1C] | O | N | CH₂ | NH | 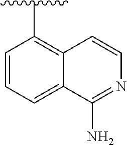 [SMILES: *c1cccc2c(N)nccc12] | H |
| 4283 | 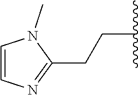 [SMILES: *CCc1nccn1C] | O | N | CH₂ | NH | 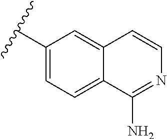 [SMILES: *c1ccc2c(N)nccc2c1] | H |
| 4284 | 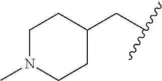 [SMILES: *CC1CCN(C)CC1] | NH | N | CH₂ | NH | 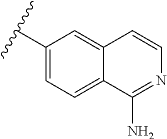 [SMILES: *c1ccc2c(N)nccc2c1] | H |
| 4285 | 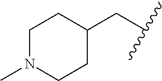 [SMILES: *CC1CCN(C)CC1] | NH | N | CH₂ | NH | 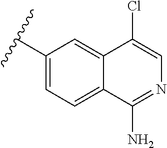 [SMILES: *c1ccc2c(N)ncc(Cl)c2c1] | H |
| 4286 | 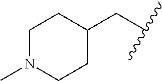 [SMILES: *CC1CCN(C)CC1] | NH | N | CH₂ | NH | 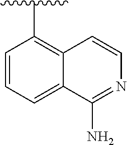 [SMILES: *c1cccc2c(N)nccc12] | H |
| 4287 | 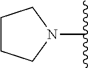 [SMILES: *N1CCCC1] | CH₂ | CH | CH₂ | NH | 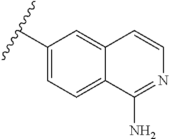 [SMILES: *c1ccc2c(N)nccc2c1] | H |
| 4288 | 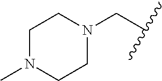 [SMILES: *CN1CCN(C)CC1] | CH₂ | CH | CH₂ | NH | 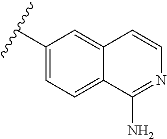 [SMILES: *c1ccc2c(N)nccc2c1] | H |
| 4289 | 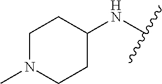 [SMILES: *NC1CCN(C)CC1] | C═O | N | CH₂ | NH | 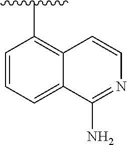 [SMILES: *c1cccc2c(N)nccc12] | H |
| 4290 | 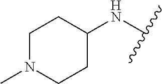 [SMILES: *NC1CCN(C)CC1] | SO₂ | N | CH₂ | NH | 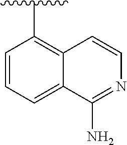 [SMILES: *c1cccc2c(N)nccc12] | H |
| 4291 | 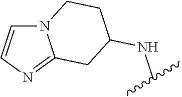 [SMILES: *NC1CCn2ccnc2C1] | SO₂ | N | CH₂ | NH | 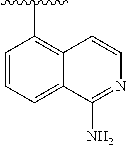 [SMILES: *c1cccc2c(N)nccc12] | H |
| 4292 | 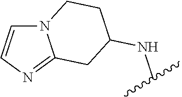 [SMILES: *NC1CCn2ccnc2C1] | SO₂ | N | CH₂ | NH | 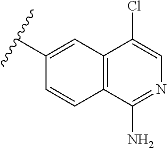 [SMILES: *c1ccc2c(N)ncc(Cl)c2c1] | H |
| 4293 | 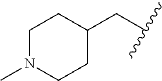 [SMILES: *CC1CCN(C)CC1] | O | N | CH₂ | NH | 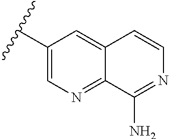 [SMILES: *c1cnc2c(N)nccc2c1] | H |
| 4294 | 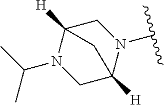 [SMILES: *N1C[C@@H]2C[C@H]1CN2C(C)C] | CH₂ | N | CH₂ | NH | 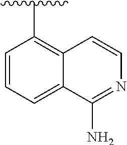 [SMILES: *c1cccc2c(N)nccc12] | H |
| 4295 | 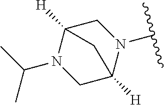 [SMILES: *N1C[C@H]2C[C@@H]1CN2C(C)C] | CH₂ | N | CH₂ | NH | 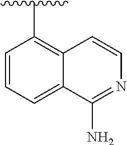 [SMILES: *c1cccc2c(N)nccc12] | H |
| 4296 | 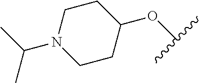 [SMILES: *OC1CCN(C(C)C)CC1] | CH₂ | N | CH₂ | NH | 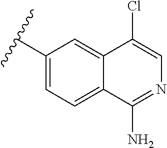 [SMILES: *c1ccc2c(N)ncc(Cl)c2c1] | H |
| 4297 | 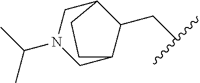 [SMILES: *CC1C2CCC1CN(C(C)C)C2] | CO | N | CH₂ | NH | 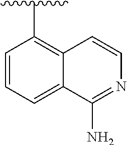 [SMILES: *c1cccc2c(N)nccc12] | H |
| 4298 | 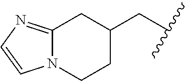 [SMILES: *CC1CCn2ccnc2C1] | O | N | CH₂ | NH | 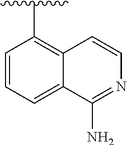 [SMILES: *c1cccc2c(N)nccc12] | H |
| 4299 | 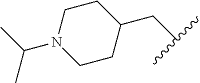 [SMILES: *CC1CCN(C(C)C)CC1] | O | N | CH₂ | NH | 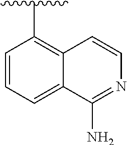 [SMILES: *c1cccc2c(N)nccc12] | H |
| 4300 | 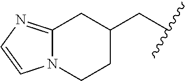 [SMILES: *CC1CCn2ccnc2C1] | O | N | CH₂ | NH | 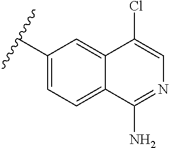 [SMILES: *c1ccc2c(N)ncc(Cl)c2c1] | H |
| 4301 | 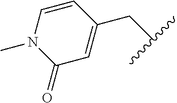 [SMILES: *Cc1ccn(C)c(=O)c1] | O | N | CH₂ | NH | 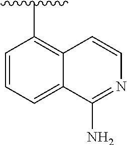 [SMILES: *c1cccc2c(N)nccc12] | H |
| 4302 | 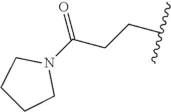 [SMILES: *CCC(=O)N1CCCC1] | NH | N | CH₂ | NH | 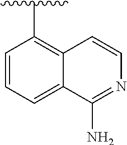 [SMILES: *c1cccc2c(N)nccc12] | H |
| 4303 | 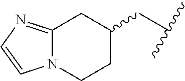 [SMILES: *CC1CCn2ccnc2C1] | NH | N | CH₂ | NH | 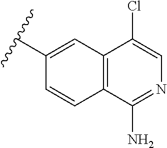 [SMILES: *c1ccc2c(N)ncc(Cl)c2c1] | H |
| 4304 | 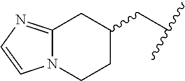 [SMILES: *CC1CCn2ccnc2C1] | NH | N | CH₂ | NH | 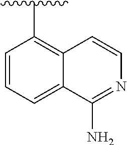 [SMILES: *c1cccc2c(N)nccc12] | H |
| 4306 | 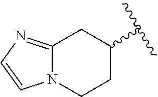 | O | N | CH₂ | NH | 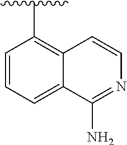 [SMILES: *c1cccc2c(N)nccc12] | H |
| 4307 | 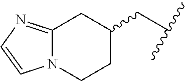 [SMILES: *CC1CCn2ccnc2C1] | O | N | CH₂ | NH | 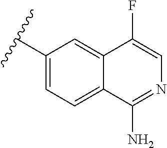 [SMILES: *c1ccc2c(N)ncc(F)c2c1] | H |
| 4308 | 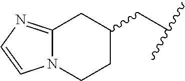 [SMILES: *CC1CCn2ccnc2C1] | O | N | CH₂ | NH | 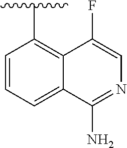 [SMILES: *c1cccc2c(N)ncc(F)c12] | H |
| 4309 | 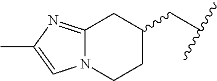 [SMILES: *CC1CCn2cc(C)nc2C1] | O | N | CH₂ | NH | 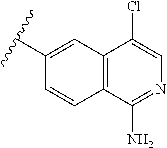 [SMILES: *c1ccc2c(N)ncc(Cl)c2c1] | H |
| 4310 | 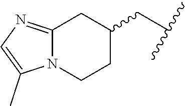 [SMILES: *CC1CCn2c(C)cnc2C1] | O | N | CH₂ | NH | 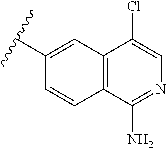 [SMILES: *c1ccc2c(N)ncc(Cl)c2c1] | H |
| 4311 | 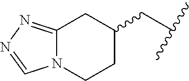 [SMILES: *CC1CCn2cnnc2C1] | O | N | CH₂ | NH | 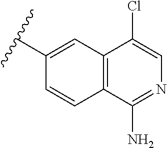 [SMILES: *c1ccc2c(N)ncc(Cl)c2c1] | H |
| 4312 | 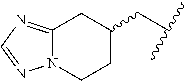 [SMILES: *CC1CCn2ncnc2C1] | O | N | CH₂ | NH | 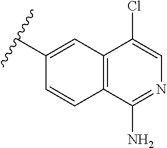 [SMILES: *c1ccc2c(N)ncc(Cl)c2c1] | H |
| 4313 | 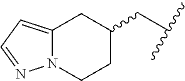 [SMILES: *CC1CCn2nccc2C1] | O | N | CH₂ | NH | 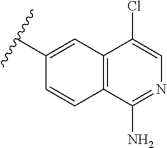 [SMILES: *c1ccc2c(N)ncc(Cl)c2c1] | H |
| 4314 | 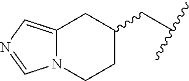 [SMILES: *CC1CCn2cncc2C1] | O | N | CH₂ | NH | 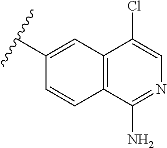 [SMILES: *c1ccc2c(N)ncc(Cl)c2c1] | H |
| 4315 | 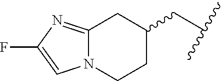 [SMILES: *CC1CCn2cc(F)nc2C1] | O | N | CH₂ | NH | 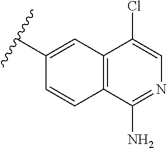 [SMILES: *c1ccc2c(N)ncc(Cl)c2c1] | H |
| 4316 | 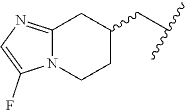 [SMILES: *CC1CCn2c(F)cnc2C1] | O | N | CH₂ | NH | 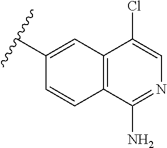 [SMILES: *c1ccc2c(N)ncc(Cl)c2c1] | H |
| 4317 | 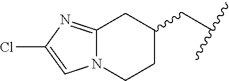 [SMILES: *CC1CCn2cc(Cl)nc2C1] | O | N | CH₂ | NH | 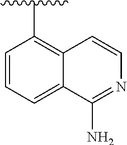 [SMILES: *c1cccc2c(N)nccc12] | H |
| 4318 | 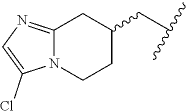 [SMILES: *CC1CCn2c(Cl)cnc2C1] | O | N | CH₂ | NH | 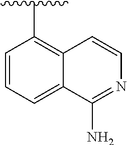 [SMILES: *c1cccc2c(N)nccc12] | H |
| 4319 | 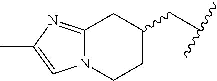 [SMILES: *CC1CCn2cc(C)nc2C1] | O | N | CH₂ | NH | 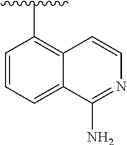 [SMILES: *c1cccc2c(N)nccc12] | H |
| 4320 | 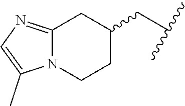 [SMILES: *CC1CCn2c(C)cnc2C1] | O | N | CH₂ | NH | 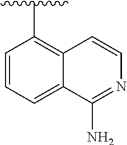 [SMILES: *c1cccc2c(N)nccc12] | H |
| 4321 | 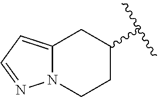 | O | N | CH₂ | NH | 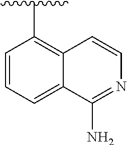 [SMILES: *c1cccc2c(N)nccc12] | H |
| 4322 | 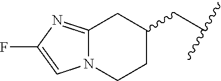 [SMILES: *CC1CCn2cc(F)nc2C1] | O | N | CH₂ | NH | 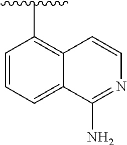 [SMILES: *c1cccc2c(N)nccc12] | H |
| 4323 | 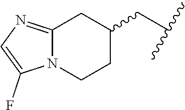 [SMILES: *CC1CCn2c(F)cnc2C1] | O | N | CH₂ | NH | 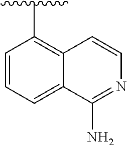 [SMILES: *c1cccc2c(N)nccc12] | H |
| 4324 | 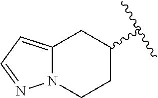 | O | N | CH₂ | NH | 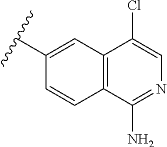 [SMILES: *c1ccc2c(N)ncc(Cl)c2c1] | H |
| 4325 | 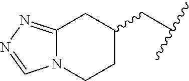 [SMILES: *CC1CCn2cnnc2C1] | O | N | CH₂ | NH | 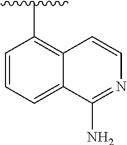 [SMILES: *c1cccc2c(N)nccc12] | H |
| 4326 | 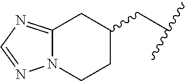 [SMILES: *CC1CCn2ncnc2C1] | O | N | CH₂ | NH | 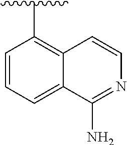 [SMILES: *c1cccc2c(N)nccc12] | H |
| 4327 | 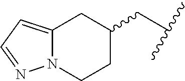 [SMILES: *CC1CCn2nccc2C1] | O | N | CH₂ | NH | 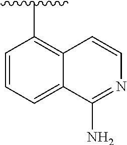 [SMILES: *c1cccc2c(N)nccc12] | H |
| 4328 | 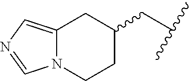 [SMILES: *CC1CCn2cncc2C1] | O | N | CH₂ | NH | 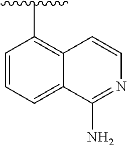 [SMILES: *c1cccc2c(N)nccc12] | H |
| 4331 | 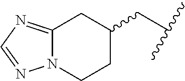 [SMILES: *CC1CCn2ncnc2C1] | O | N | CH₂ | NH | 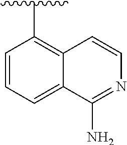 [SMILES: *c1cccc2c(N)nccc12] | H |
| 4332 | 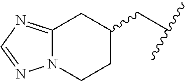 [SMILES: *CC1CCn2ncnc2C1] | O | N | CH₂ | NH | 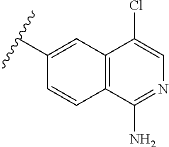 [SMILES: *c1ccc2c(N)ncc(Cl)c2c1] | H |
| 4334 | 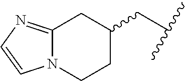 [SMILES: *CC1CCn2ccnc2C1] | NH | N | CH₂ | NH | 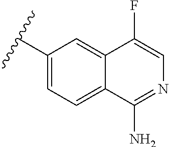 [SMILES: *c1ccc2c(N)ncc(F)c2c1] | H |
| 4335 | 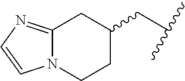 [SMILES: *CC1CCn2ccnc2C1] | NH | N | CH₂ | NH | 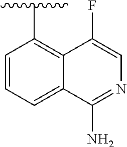 [SMILES: *c1cccc2c(N)ncc(F)c12] | H |
| 4336 | 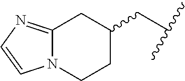 [SMILES: *CC1CCn2ccnc2C1] | NH | N | CH₂ | NH | 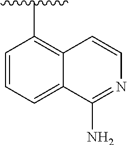 [SMILES: *c1cccc2c(N)nccc12] | H |
| 4337 | 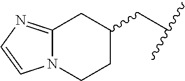 [SMILES: *CC1CCn2ccnc2C1] | O | N | CH₂ | NH | 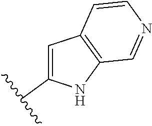 [SMILES: *c1cc2ccncc2[nH]1] | H |
| 4338 | 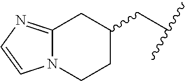 [SMILES: *CC1CCn2ccnc2C1] | O | N | CH₂ | NH | 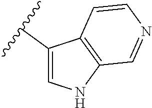 [SMILES: *c1c[nH]c2cnccc12] | H |
| 4339 | 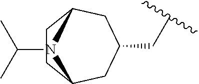 [SMILES: *C[C@H]1C[C@H]2CC[C@@H](C1)N2C(C)C] | O | N | CH₂ | NH | 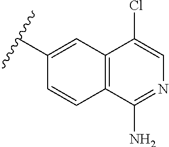 [SMILES: *c1ccc2c(N)ncc(Cl)c2c1] | H |
| 4340 | 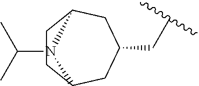 | O | N | CH₂ | NH | 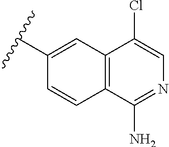 [SMILES: *c1ccc2c(N)ncc(Cl)c2c1] | H |
| 4341 | 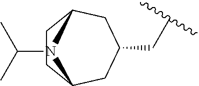 [SMILES: *C[C@H]1C[C@H]2CC[C@@H](C1)N2C(C)C] | O | N | CH₂ | NH | 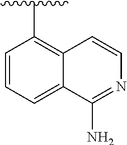 [SMILES: *c1cccc2c(N)nccc12] | H |
| 4342 | 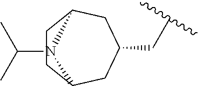 | O | N | CH₂ | NH | 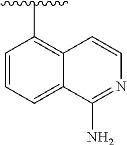 [SMILES: *c1cccc2c(N)nccc12] | H |
| 4343 | 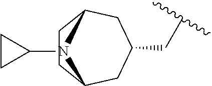 [SMILES: *C[C@H]1C[C@H]2CC[C@@H](C1)N2C1CC1] | O | N | CH₂ | NH | 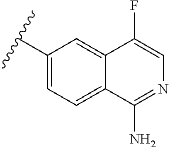 [SMILES: *c1ccc2c(N)ncc(F)c2c1] | H |
| 4344 | 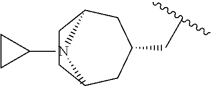 | O | N | CH₂ | NH | 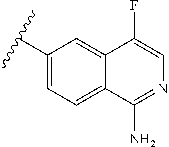 [SMILES: *c1ccc2c(N)ncc(F)c2c1] | H |
| 4345 | 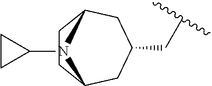 [SMILES: *C[C@H]1C[C@H]2CC[C@@H](C1)N2C1CC1] | O | N | CH₂ | NH | 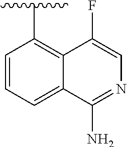 [SMILES: *c1cccc2c(N)ncc(F)c12] | H |
| 4346 | 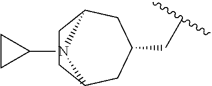 | O | N | CH₂ | NH | 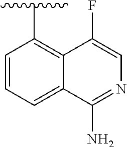 [SMILES: *c1cccc2c(N)ncc(F)c12] | H |
| 4347 | 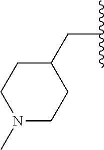 [SMILES: *CC1CCN(C)CC1] | O | N | CH₂ | NH | 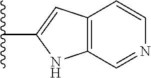 [SMILES: *c1cc2ccncc2[nH]1] | H |
| 4348 | 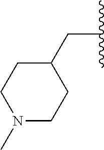 [SMILES: *CC1CCN(C)CC1] | O | N | CH₂ | NH | 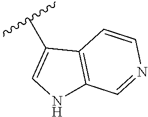 [SMILES: *c1c[nH]c2cnccc12] | H |
| 4349 | 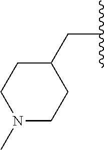 [SMILES: *CC1CCN(C)CC1] | O | N | CH₂ | NH | 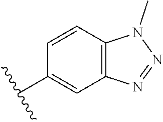 [SMILES: *c1ccc2c(c1)nnn2C] | H |
| 4350 | 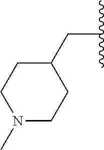 [SMILES: *CC1CCN(C)CC1] | O | N | CH₂ | NH | 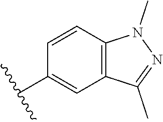 [SMILES: *c1ccc2c(c1)c(C)nn2C] | H |
| 4351 | 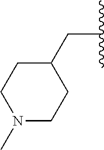 [SMILES: *CC1CCN(C)CC1] | O | N | CH₂ | NH | 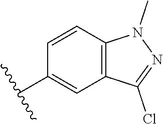 [SMILES: *c1ccc2c(c1)c(Cl)nn2C] | H |
| 4352 | 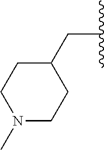 [SMILES: *CC1CCN(C)CC1] | O | N | CH₂ | NH | 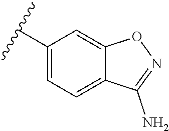 [SMILES: *c1ccc2c(N)noc2c1] | H |
| 4353 | 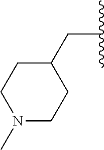 [SMILES: *CC1CCN(C)CC1] | O | N | CH₂ | NH | 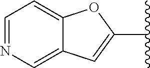 [SMILES: *c1cc2cnccc2o1] | H |
| 4354 | 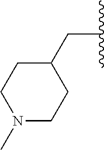 [SMILES: *CC1CCN(C)CC1] | O | N | CH₂ | NH | 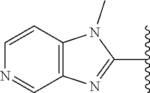 [SMILES: *c1nc2cnccc2n1C] | H |
| 4355 | 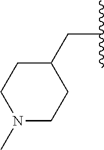 [SMILES: *CC1CCN(C)CC1] | O | N | CH₂ | NH | 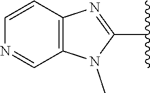 [SMILES: *c1nc2ccncc2n1C] | H |
| 4356 | 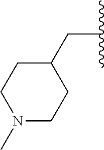 [SMILES: *CC1CCN(C)CC1] | O | N | CH₂ | NH | 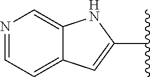 [SMILES: *c1cc2ccncc2[nH]1] | H |
| 4357 | 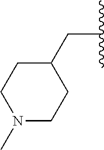 [SMILES: *CC1CCN(C)CC1] | O | N | CH₂ | NH | 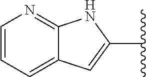 [SMILES: *c1cc2cccnc2[nH]1] | H |
| 4358 | 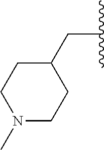 [SMILES: *CC1CCN(C)CC1] | O | N | CH₂ | NH | 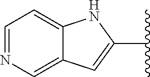 [SMILES: *c1cc2cnccc2[nH]1] | H |
| 4359 | 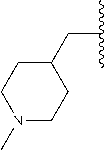 [SMILES: *CC1CCN(C)CC1] | O | N | CH₂ | NH | 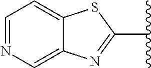 [SMILES: *c1nc2cnccc2s1] | H |
| 4360 | 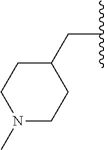 [SMILES: *CC1CCN(C)CC1] | O | N | CH₂ | NH | 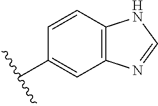 [SMILES: *c1ccc2[nH]cnc2c1] | H |
| 4361 | 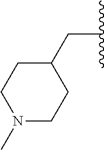 [SMILES: *CC1CCN(C)CC1] | O | N | CH₂ | NH | 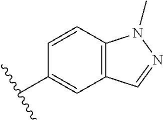 [SMILES: *c1ccc2c(cnn2C)c1] | H |
| 4362 | 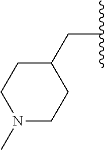 [SMILES: *CC1CCN(C)CC1] | O | N | CH₂ | NH | 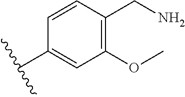 [SMILES: *c1ccc(CN)c(OC)c1] | H |
| 4363 | 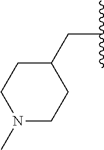 [SMILES: *CC1CCN(C)CC1] | O | N | CH₂ | NH | 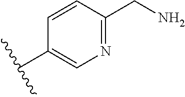 [SMILES: *c1ccc(CN)nc1] | H |
| 4364 | 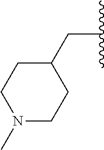 [SMILES: *CC1CCN(C)CC1] | O | N | CH₂ | NH | 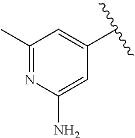 [SMILES: *c1cc(C)nc(N)c1] | H |
| 4365 | 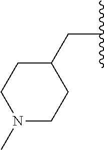 [SMILES: *CC1CCN(C)CC1] | O | N | CH₂ | NH | 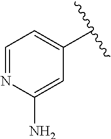 [SMILES: *c1ccnc(N)c1] | H |
| 4366 | 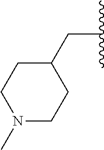 [SMILES: *CC1CCN(C)CC1] | O | N | CH₂ | NH | 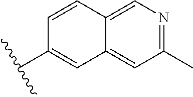 [SMILES: *c1ccc2cnc(C)cc2c1] | H |
| 4367 | 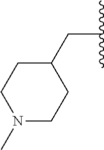 [SMILES: *CC1CCN(C)CC1] | O | N | CH₂ | NH | 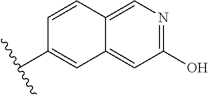 [SMILES: *c1ccc2cnc(O)cc2c1] | H |
| 4368 | 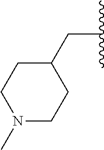 [SMILES: *CC1CCN(C)CC1] | O | N | CH₂ | NH | 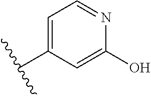 [SMILES: *c1ccnc(O)c1] | H |
| 4369 | 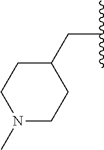 [SMILES: *CC1CCN(C)CC1] | O | N | CH₂ | NH | 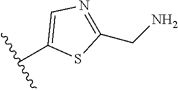 [SMILES: *c1cnc(CN)s1] | H |
| 4370 | 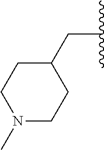 [SMILES: *CC1CCN(C)CC1] | O | N | CH₂ | NH | 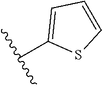 [SMILES: *c1cccs1] | H |
| 4371 | 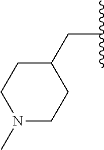 [SMILES: *CC1CCN(C)CC1] | O | N | CH₂ | NH | 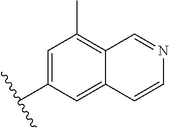 [SMILES: *c1cc(C)c2cnccc2c1] | H |
| 4372 | 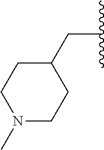 [SMILES: *CC1CCN(C)CC1] | O | N | CH₂ | NH | 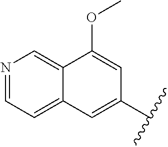 [SMILES: *c1cc(OC)c2cnccc2c1] | H |
| 4373 | 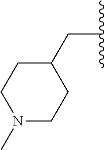 [SMILES: *CC1CCN(C)CC1] | O | N | CH₂ | NH | 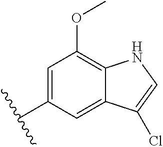 [SMILES: *c1cc(OC)c2[nH]cc(Cl)c2c1] | H |
| 4374 | 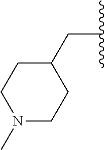 [SMILES: *CC1CCN(C)CC1] | O | N | CH₂ | NH | 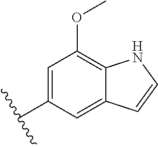 [SMILES: *c1cc(OC)c2[nH]ccc2c1] | H |
| 4375 | 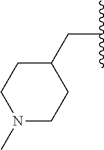 [SMILES: *CC1CCN(C)CC1] | O | N | CH₂ | NH | 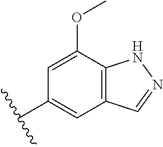 [SMILES: *c1cc(OC)c2[nH]ncc2c1] | H |
| 4376 | 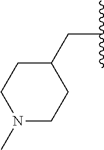 [SMILES: *CC1CCN(C)CC1] | O | N | CH₂ | NH | 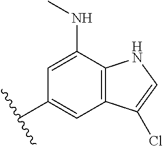 [SMILES: *c1cc(NC)c2[nH]cc(Cl)c2c1] | H |
| 4377 | 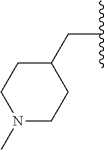 [SMILES: *CC1CCN(C)CC1] | O | N | CH₂ | NH | 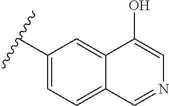 [SMILES: *c1ccc2cncc(O)c2c1] | H |
| 4378 | 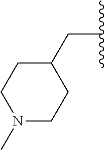 [SMILES: *CC1CCN(C)CC1] | O | N | CH₂ | NH | 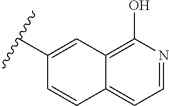 [SMILES: *c1ccc2ccnc(O)c2c1] | H |
| 4379 | 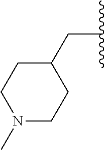 [SMILES: *CC1CCN(C)CC1] | O | N | CH₂ | NH | 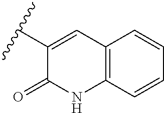 [SMILES: *c1cc2ccccc2[nH]c1=O] | H |
| 4380 | 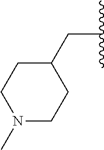 [SMILES: *CC1CCN(C)CC1] | O | N | CH₂ | NH | 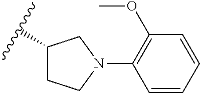 [SMILES: *[C@H]1CCN(c2ccccc2OC)C1] | H |
| 4381 | 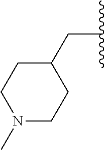 [SMILES: *CC1CCN(C)CC1] | O | N | CH₂ | NH | 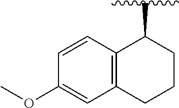 [SMILES: *[C@H]1CCCc2cc(OC)ccc21] | H |
| 4382 | 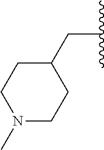 [SMILES: *CC1CCN(C)CC1] | O | N | CH₂ | NH | 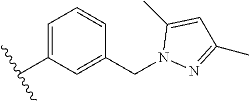 [SMILES: *c1cccc(Cn2nc(C)cc2C)c1] | H |
| 4383 | 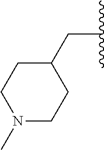 [SMILES: *CC1CCN(C)CC1] | O | N | CH₂ | NH | 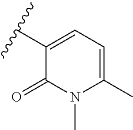 [SMILES: *c1ccc(C)n(C)c1=O] | H |
| 4384 | 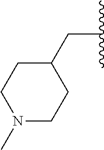 [SMILES: *CC1CCN(C)CC1] | O | N | CH₂ | NH | 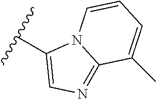 [SMILES: *c1cnc2c(C)cccn12] | H |
| 4385 | 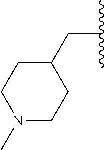 [SMILES: *CC1CCN(C)CC1] | O | N | CH₂ | NH | 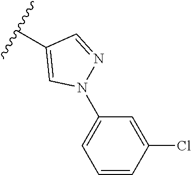 [SMILES: *c1cnn(-c2cccc(Cl)c2)c1] | H |
| 4386 | 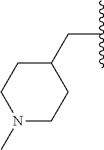 [SMILES: *CC1CCN(C)CC1] | O | N | CH₂ | NH | 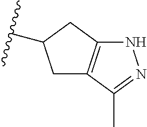 [SMILES: *C1Cc2[nH]nc(C)c2C1] | H |
| 4387 | 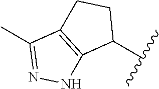 [SMILES: *C1CCc2c(C)n[nH]c21] | NH | N | CH₂ | NH | 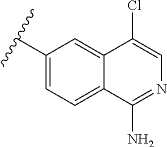 [SMILES: *c1ccc2c(N)ncc(Cl)c2c1] | H |
| 4388 | 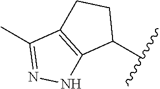 [SMILES: *C1CCc2c(C)n[nH]c21] | NH | N | CH₂ | NH | 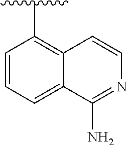 [SMILES: *c1cccc2c(N)nccc12] | H |
| 4389 | 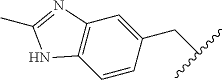 [SMILES: *Cc1ccc2[nH]c(C)nc2c1] | NH | N | CH₂ | NH | 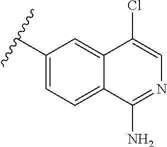 [SMILES: *c1ccc2c(N)ncc(Cl)c2c1] | H |
| 4390 | 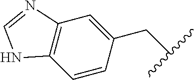 [SMILES: *Cc1ccc2[nH]cnc2c1] | NH | N | CH₂ | NH | 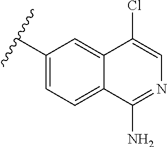 [SMILES: *c1ccc2c(N)ncc(Cl)c2c1] | H |
| 4391 | 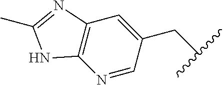 [SMILES: *Cc1cnc2[nH]c(C)nc2c1] | NH | N | CH₂ | NH | 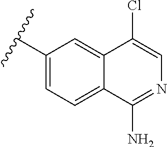 [SMILES: *c1ccc2c(N)ncc(Cl)c2c1] | H |
| 4392 | 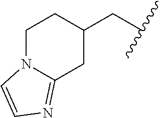 [SMILES: *CC1CCn2ccnc2C1] | O | N | CH₂ | NH | 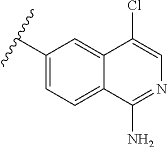 [SMILES: *c1ccc2c(N)ncc(Cl)c2c1] | CH₃ |
| 4393 | 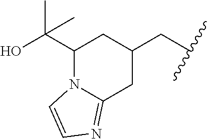 [SMILES: *CC1Cc2nccn2C(C(C)(C)O)C1] | O | N | CH₂ | NH | 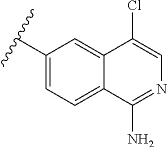 [SMILES: *c1ccc2c(N)ncc(Cl)c2c1] | H |
| 4394 | 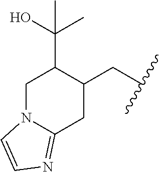 [SMILES: *CC1Cc2nccn2CC1C(C)(C)O] | O | N | CH₂ | NH | 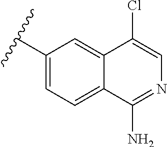 [SMILES: *c1ccc2c(N)ncc(Cl)c2c1] | H |
| 4395 | 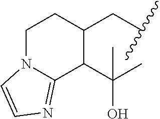 [SMILES: *CC1CCn2ccnc2C1C(C)(C)O] | O | N | CH₂ | NH | 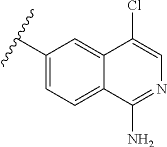 [SMILES: *c1ccc2c(N)ncc(Cl)c2c1] | H |
| 4396 | 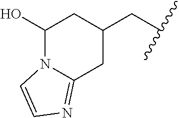 [SMILES: *CC1Cc2nccn2C(O)C1] | O | N | CH₂ | NH | 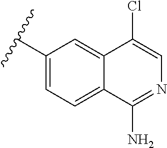 [SMILES: *c1ccc2c(N)ncc(Cl)c2c1] | H |
| 4397 | 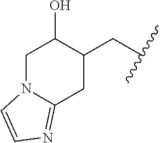 [SMILES: *CC1Cc2nccn2CC1O] | O | N | CH₂ | NH | 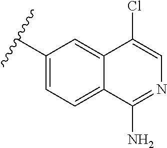 [SMILES: *c1ccc2c(N)ncc(Cl)c2c1] | H |
| 4398 | 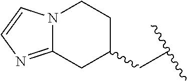 [SMILES: *CC1CCn2ccnc2C1] | NH | N | SO₂ | NH | 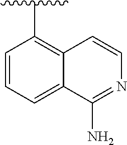 [SMILES: *c1cccc2c(N)nccc12] | H |
| 4399 | 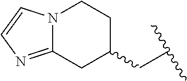 [SMILES: *CC1CCn2ccnc2C1] | O | N | SO₂ | NH | 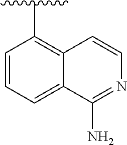 [SMILES: *c1cccc2c(N)nccc12] | H |
| 4400 | 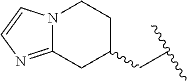 [SMILES: *CC1CCn2ccnc2C1] | O | N | CH₂ | CH₂ | 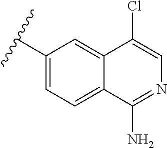 [SMILES: *c1ccc2c(N)ncc(Cl)c2c1] | H |
| 4401 | 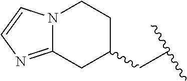 [SMILES: *CC1CCn2ccnc2C1] | O | N | CH₂ | CH₂ | 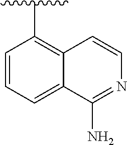 [SMILES: *c1cccc2c(N)nccc12] | H |
| 4402 | 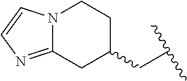 [SMILES: *CC1CCn2ccnc2C1] | O | N | CH₂ | O | 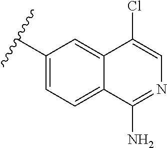 [SMILES: *c1ccc2c(N)ncc(Cl)c2c1] | H |
| 4403 | 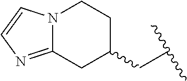 [SMILES: *CC1CCn2ccnc2C1] | O | N | CH₂ | O | 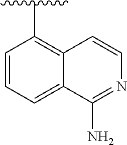 [SMILES: *c1cccc2c(N)nccc12] | H |
| 4404 | 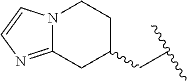 [SMILES: *CC1CCn2ccnc2C1] | O | N | CH(CH₂F) | NH | 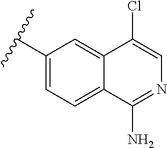 [SMILES: *c1ccc2c(N)ncc(Cl)c2c1] | H |
| 4405 | 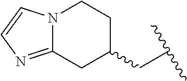 [SMILES: *CC1CCn2ccnc2C1] | O | N | CH(CH₂F) | CH₂ | 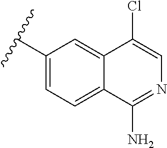 [SMILES: *c1ccc2c(N)ncc(Cl)c2c1] | H |
| 4406 | 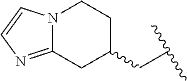 [SMILES: *CC1CCn2ccnc2C1] | O | N | CH(CH₂F) | NH | 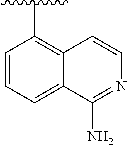 [SMILES: *c1cccc2c(N)nccc12] | H |
| 4407 | 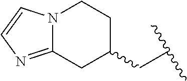 [SMILES: *CC1CCn2ccnc2C1] | O | N | CH(CH₂F) | CH₂ | 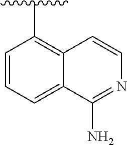 [SMILES: *c1cccc2c(N)nccc12] | H |
| 4408 | 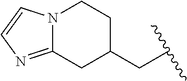 [SMILES: *CC1CCn2ccnc2C1] | O | N | CH₂ | NH | 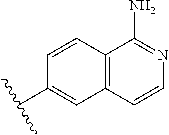 [SMILES: *c1ccc2c(N)nccc2c1] | H |
| 4409 | 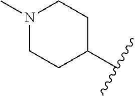 [SMILES: *C1CCN(C)CC1] | CH₂CH₂ | N | CH₂ | NH | 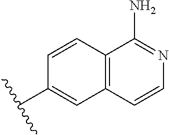 [SMILES: *c1ccc2c(N)nccc2c1] | H |
| 4410 | 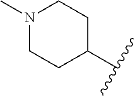 [SMILES: *C1CCN(C)CC1] | CH₂CH₂ | CH | CH₂ | NH | 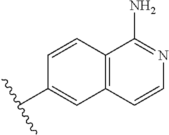 [SMILES: *c1ccc2c(N)nccc2c1] | H |
| 4411 | 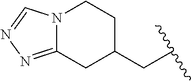 [SMILES: *CC1CCn2cnnc2C1] | O | N | CH₂ | NH | 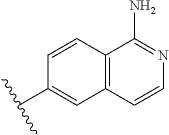 [SMILES: *c1ccc2c(N)nccc2c1] | H |
| 4412 | 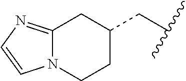 [SMILES: *C[C@@H]1CCn2ccnc2C1] | O | N | CH₂ | NH | 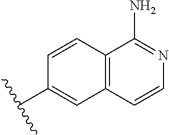 [SMILES: *c1ccc2c(N)nccc2c1] | H |
| 4413 | 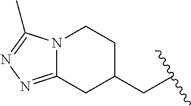 [SMILES: *CC1CCn2c(C)nnc2C1] | O | N | CH₂ | NH | 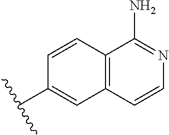 [SMILES: *c1ccc2c(N)nccc2c1] | H |
| 4414 | 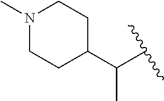 [SMILES: *C(C)C1CCN(C)CC1] | O | N | CH₂ | NH | 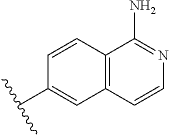 [SMILES: *c1ccc2c(N)nccc2c1] | H |
| 4415 | 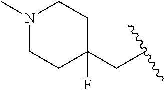 [SMILES: *CC1(F)CCN(C)CC1] | O | N | CH₂ | NH | 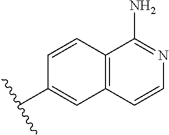 [SMILES: *c1ccc2c(N)nccc2c1] | H |
| 4416 | 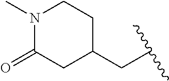 [SMILES: *CC1CCN(C)C(=O)C1] | O | N | CH₂ | NH | 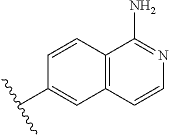 [SMILES: *c1ccc2c(N)nccc2c1] | H |
| 4417 | 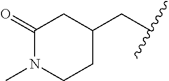 [SMILES: *CC1CCN(C)C(=O)C1] | O | N | CH₂ | NH | 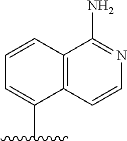 [SMILES: *c1cccc2c(N)nccc12] | H |
| 4418 | 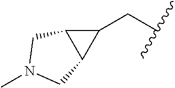 [SMILES: *CC1[C@H]2CN(C)C[C@@H]12] | O | N | CH₂ | NH | 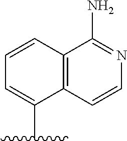 [SMILES: *c1cccc2c(N)nccc12] | H |
| 4419 | 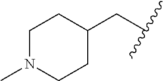 [SMILES: *CC1CCN(C)CC1] | O | N | CH₂ | NCH3 | 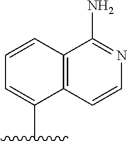 [SMILES: *c1cccc2c(N)nccc12] | H |
| 4420 | 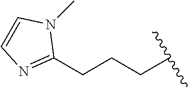 [SMILES: *CCCc1nccn1C] | O | N | CH₂ | NH | 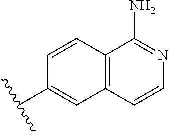 [SMILES: *c1ccc2c(N)nccc2c1] | H |
| 4421 | 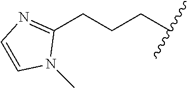 [SMILES: *CCCc1nccn1C] | O | N | CH₂ | NH | 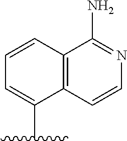 [SMILES: *c1cccc2c(N)nccc12] | H |
| 4422 | 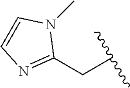 [SMILES: *Cc1nccn1C] | O | N | CH₂ | NH | 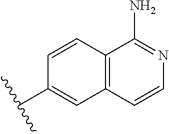 [SMILES: *c1ccc2c(N)nccc2c1] | H |
| 4423 | 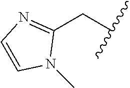 [SMILES: *Cc1nccn1C] | O | N | CH₂ | NH | 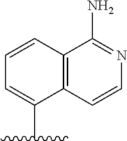 [SMILES: *c1cccc2c(N)nccc12] | H |
| 4424 | 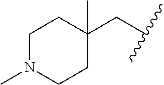 [SMILES: *CC1(C)CCN(C)CC1] | O | N | CH₂ | NH | 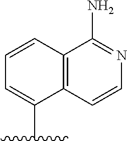 [SMILES: *c1cccc2c(N)nccc12] | H |
| 4425 | 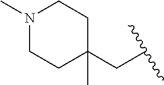 [SMILES: *CC1(C)CCN(C)CC1] | O | N | CH₂ | NH | 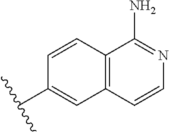 [SMILES: *c1ccc2c(N)nccc2c1] | H |
| 4426 | 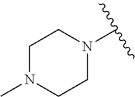 [SMILES: *N1CCN(C)CC1] | absent | N | CH₂ | NH | 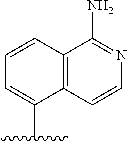 [SMILES: *c1cccc2c(N)nccc12] | H |
| 4427 | 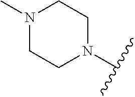 [SMILES: *N1CCN(C)CC1] | absent | N | CH₂ | NH | 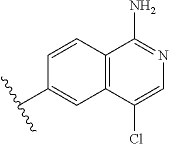 [SMILES: *c1ccc2c(N)ncc(Cl)c2c1] | H |
| 4428 | 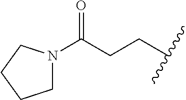 [SMILES: *CCC(=O)N1CCCC1] | NH | N | CH₂ | NH | 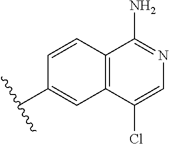 [SMILES: *c1ccc2c(N)ncc(Cl)c2c1] | H |
| 4429 | 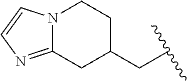 [SMILES: *CC1CCn2ccnc2C1] | O | N | CH₂ | NH | 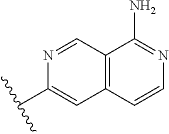 [SMILES: *c1cc2ccnc(N)c2cn1] | H |
| 4430 | 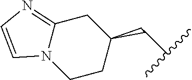 [SMILES: *C[C@H]1CCn2ccnc2C1] | O | N | CH₂ | NH | 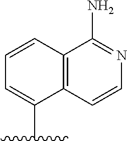 [SMILES: *c1cccc2c(N)nccc12] | H |
| 4431 | 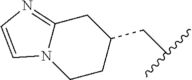 [SMILES: *C[C@@H]1CCn2ccnc2C1] | O | N | CH₂ | NH | 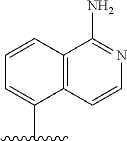 [SMILES: *c1cccc2c(N)nccc12] | H |
| 4432 | 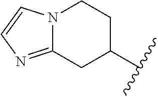 [SMILES: *C1CCn2ccnc2C1] | O | N | CH₂ | NH | 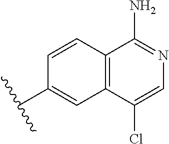 [SMILES: *c1ccc2c(N)ncc(Cl)c2c1] | H |
| 4433 | 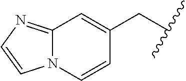 [SMILES: *Cc1ccn2ccnc2c1] | O | N | CH₂ | NH | 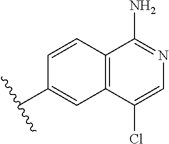 [SMILES: *c1ccc2c(N)ncc(Cl)c2c1] | H |

TABLE 4b

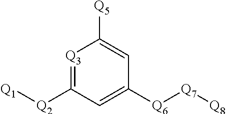

| Example | | | | | | | |
|---|---|---|---|---|---|---|---|
| Number | Q₁ | Q₂ | Q₃ | Q₆ | Q₇ | Q₈ | Q₅ |
| 4434 | 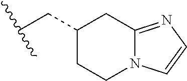 [SMILES: *C[C@H]1CCn2ccnc2C1] | NH | N | CH₂ | NH | 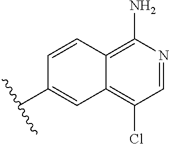 [SMILES: *c1ccc2c(N)ncc(Cl)c2c1] | H |
| 4435 | 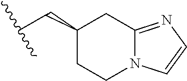 [SMILES: *C[C@@H]1CCn2ccnc2C1] | NH | N | CH₂ | NH | 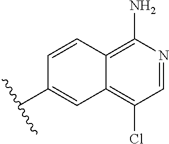 [SMILES: *c1ccc2c(N)ncc(Cl)c2c1] | H |
| 4436 | 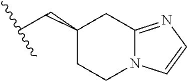 [SMILES: *C[C@@H]1CCn2ccnc2C1] | O | N | CH₂ | NH | 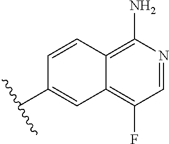 [SMILES: *c1ccc2c(N)ncc(F)c2c1] | H |
| 4437 | 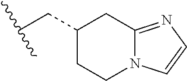 [SMILES: *C[C@H]1CCn2ccnc2C1] | O | N | CH₂ | NH | 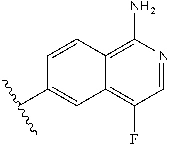 [SMILES: *c1ccc2c(N)ncc(F)c2c1] | H |
| 4438 | 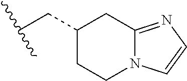 [SMILES: *C[C@H]1CCn2ccnc2C1] | O | N | CH₂ | NH | 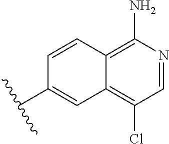 [SMILES: *c1ccc2c(N)ncc(Cl)c2c1] | H |
| 4439 | 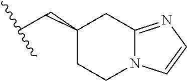 [SMILES: *C[C@@H]1CCn2ccnc2C1] | O | N | CH₂ | NH | 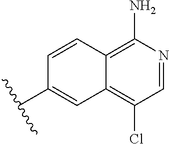 [SMILES: *c1ccc2c(N)ncc(Cl)c2c1] | H |
| 4440 | 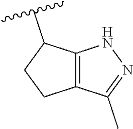 [SMILES: *C1CCc2c(C)n[nH]c21] | NH | N | CH₂ | NH | 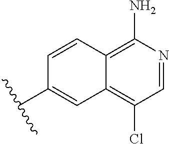 [SMILES: *c1ccc2c(N)ncc(Cl)c2c1] | H |
| 4441 | 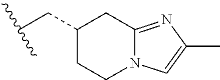 [SMILES: *C[C@H]1CCn2cc(C)nc2C1] | O | N | CH₂ | NH | 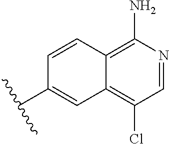 [SMILES: *c1ccc2c(N)ncc(Cl)c2c1] | H |
| 4442 | 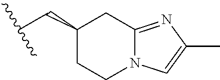 [SMILES: *C[C@@H]1CCn2cc(C)nc2C1] | O | N | CH₂ | NH | 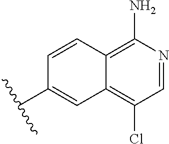 [SMILES: *c1ccc2c(N)ncc(Cl)c2c1] | H |
| 4443 | 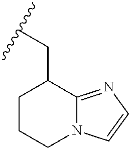 [SMILES: *CC1CCCn2ccnc21] | O | N | CH₂ | NH | 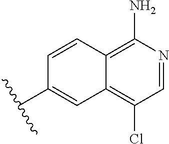 [SMILES: *c1ccc2c(N)ncc(Cl)c2c1] | H |
| 4444 | 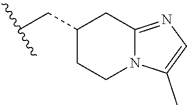 [SMILES: *C[C@H]1CCn2c(C)cnc2C1] | O | N | CH₂ | NH | 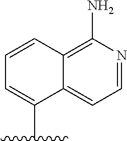 [SMILES: *c1cccc2c(N)nccc12] | H |
| 4445 | 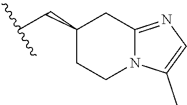 [SMILES: *C[C@@H]1CCn2c(C)cnc2C1] | O | N | CH₂ | NH | 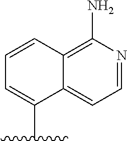 [SMILES: *c1cccc2c(N)nccc12] | H |
| 4446 | 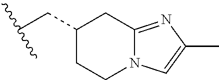 [SMILES: *C[C@H]1CCn2cc(C)nc2C1] | O | N | CH₂ | NH | 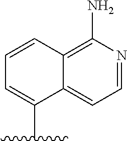 [SMILES: *c1cccc2c(N)nccc12] | H |
| 4447 | 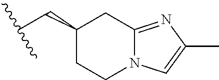 [SMILES: *C[C@@H]1CCn2cc(C)nc2C1] | O | N | CH₂ | NH | 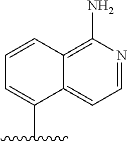 [SMILES: *c1cccc2c(N)nccc12] | H |
| 4448 | 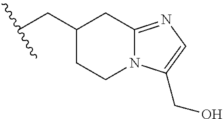 [SMILES: *CC1CCn2c(CO)cnc2C1] | O | N | CH₂ | NH | 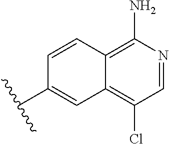 [SMILES: *c1ccc2c(N)ncc(Cl)c2c1] | H |
| 4449 | 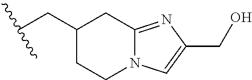 [SMILES: *CC1CCn2cc(CO)nc2C1] | O | N | CH₂ | NH | 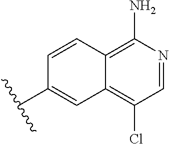 [SMILES: *c1ccc2c(N)ncc(Cl)c2c1] | H |
| 4450 | 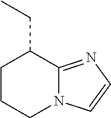 | O | N | CH₂ | NH | 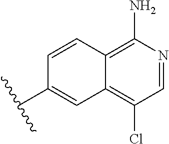 [SMILES: *c1ccc2c(N)ncc(Cl)c2c1] | H |
| 4451 | 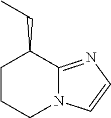 | O | N | CH₂ | NH | 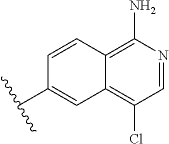 [SMILES: *c1ccc2c(N)ncc(Cl)c2c1] | H |
| 4452 | 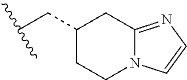 [SMILES: *C[C@H]1CCn2ccnc2C1] | O | N | CH₂ | NH | 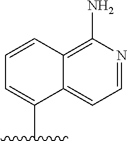 [SMILES: *c1cccc2c(N)nccc12] | H |
| 4453 | 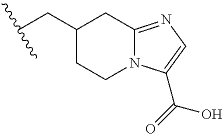 [SMILES: *CC1CCn2c(C(=O)O)cnc2C1] | O | N | CH₂ | NH | 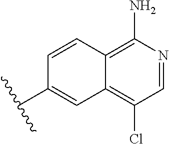 [SMILES: *c1ccc2c(N)ncc(Cl)c2c1] | H |
| 4454 | 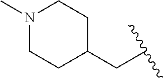 [SMILES: *CC1CCN(C)CC1] | O | N | CH₂ | NH | 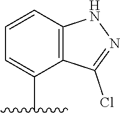 [SMILES: *c1cccc2[nH]nc(Cl)c12] | H |
| 4455 | 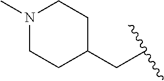 [SMILES: *CC1CCN(C)CC1] | O | N | CH₂ | NH | 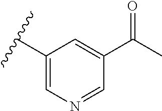 [SMILES: *c1cncc(C(C)=O)c1] | H |
| 4456 | 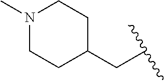 [SMILES: *CC1CCN(C)CC1] | O | N | CH₂ | NH | 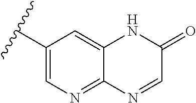 [SMILES: *c1cnc2ncc(=O)[nH]c2c1] | H |
| 4457 | 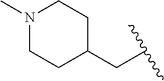 [SMILES: *CC1CCN(C)CC1] | O | N | CH₂ | NH | 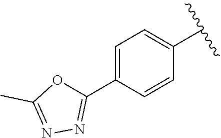 [SMILES: *c1ccc(-c2nnc(C)o2)cc1] | H |
| 4458 | 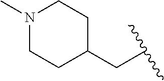 [SMILES: *CC1CCN(C)CC1] | O | N | CH₂ | NH | 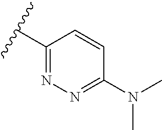 [SMILES: *c1ccc(N(C)C)nn1] | H |
| 4459 | 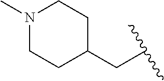 [SMILES: *CC1CCN(C)CC1] | O | N | CH₂ | NH | 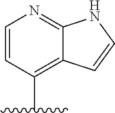 [SMILES: *c1ccnc2[nH]ccc12] | H |
| 4460 | 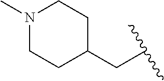 [SMILES: *CC1CCN(C)CC1] | O | N | CH₂ | NH | 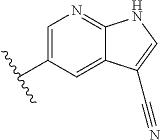 [SMILES: *c1cnc2[nH]cc(C#N)c2c1] | H |
| 4461 | 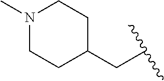 [SMILES: *CC1CCN(C)CC1] | O | N | CH₂ | NH | 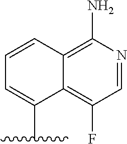 [SMILES: *c1cccc2c(N)ncc(F)c12] | H |
| 4462 | 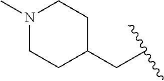 [SMILES: *CC1CCN(C)CC1] | O | N | CH₂ | NH | 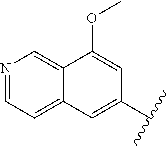 [SMILES: *c1cc(OC)c2cnccc2c1] | H |
| 4463 | 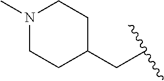 [SMILES: *CC1CCN(C)CC1] | O | N | CH₂ | NH | 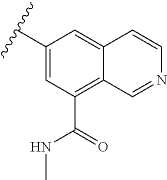 [SMILES: *c1cc(C(=O)NC)c2cnccc2c1] | H |
| 4464 | 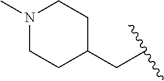 [SMILES: *CC1CCN(C)CC1] | O | N | CH₂ | NH | 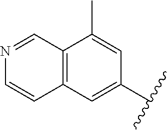 [SMILES: *c1cc(C)c2cnccc2c1] | H |
| 4465 | 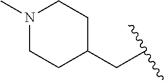 [SMILES: *CC1CCN(C)CC1] | O | N | CH₂ | NH | 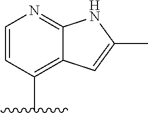 [SMILES: *c1ccnc2[nH]c(C)cc12] | H |
| 4466 | 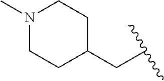 [SMILES: *CC1CCN(C)CC1] | O | N | CH₂ | NH | 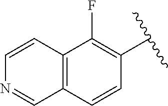 [SMILES: *c1ccc2cnccc2c1F] | H |
| 4467 | 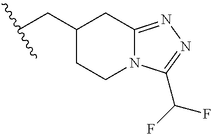 [SMILES: *CC1CCn2c(nnc2C(F)F)C1] | O | N | CH₂ | NH | 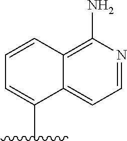 [SMILES: *c1cccc2c(N)nccc12] | H |
| 4468 | 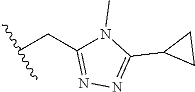 [SMILES: *Cc1nnc(C2CC2)n1C] | O | N | CH₂ | NH | 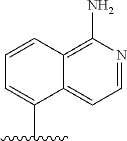 [SMILES: *c1cccc2c(N)nccc12] | H |
| 4469 | 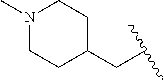 [SMILES: *CC1CCN(C)CC1] | O | N | CH₂ | NH | 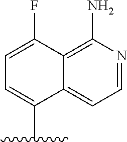 [SMILES: *c1ccc(F)c2c(N)nccc12] | H |
| 4470 | 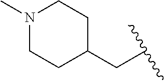 [SMILES: *CC1CCN(C)CC1] | O | N | CH₂ | NH | 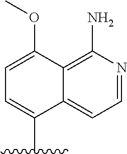 [SMILES: *c1ccc(OC)c2c(N)nccc12] | H |
| 4471 | 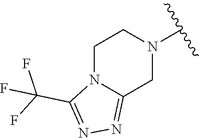 [SMILES: *N1CCn2c(nnc2C(F)(F)F)C1] | Absent | N | CH₂ | NH | 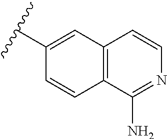 [SMILES: *c1ccc2c(N)nccc2c1] | H |
| 4472 | 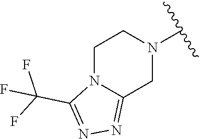 [SMILES: *N1CCn2c(nnc2C(F)(F)F)C1] | Absent | N | CH₂ | NH | 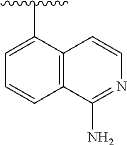 [SMILES: *c1cccc2c(N)nccc12] | H |

TABLE 5a

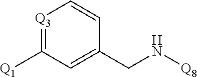

| Example | | | |
|---|---|---|---|
| Number | Q₁ | Q₃ | Q₈ |
| 5434 | 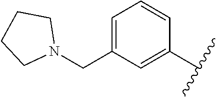 [SMILES: *c1cccc(CN2CCCC2)c1] | N | 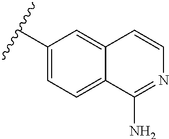 [SMILES: *c1ccc2c(N)nccc2c1] |
| 5435 | 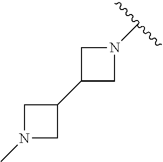 [SMILES: *N1CC(C2CN(C)C2)C1] | N | 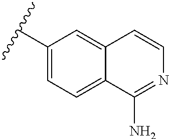 [SMILES: *c1ccc2c(N)nccc2c1] |
| 5436 | 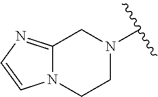 [SMILES: *N1CCn2ccnc2C1] | N | 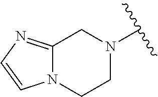 [SMILES: *N1CCn2ccnc2C1] |
| 5437 | 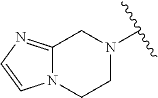 [SMILES: *N1CCn2ccnc2C1] | N | 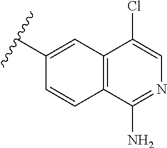 [SMILES: *c1ccc2c(N)ncc(Cl)c2c1] |
| 5438 | 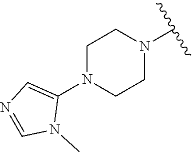 [SMILES: *N1CCN(c2cncn2C)CC1] | N | 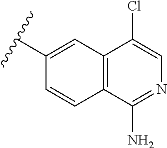 [SMILES: *c1ccc2c(N)ncc(Cl)c2c1] |
| 5439 | 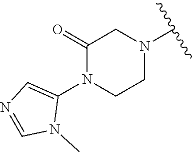 [SMILES: *N1CCN(c2cncn2C)C(=O)C1] | N | 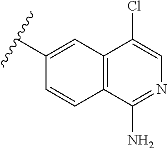 [SMILES: *c1ccc2c(N)ncc(Cl)c2c1] |
| 5440 | 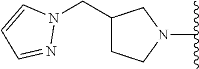 [SMILES: *N1CCC(Cn2cccn2)C1] | N | 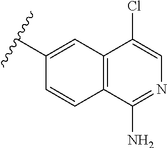 [SMILES: *c1ccc2c(N)ncc(Cl)c2c1] |
| 5441 | 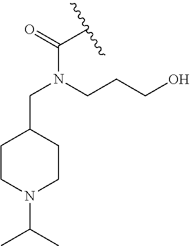 [SMILES: *C(=O)N(CCCO)CC1CCN(C(C)C)CC1] | CH | 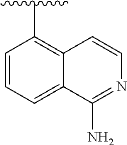 [SMILES: *c1cccc2c(N)nccc12] |

TABLE 5b

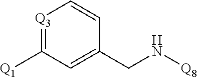

| Example | | | |
|---|---|---|---|
| Number | Q₁ | Q₃ | Q₈ |
| 5442 | 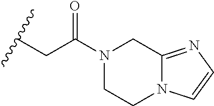 [SMILES: *CC(=O)N1CCn2ccnc2C1] | CH | 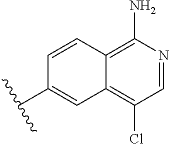 [SMILES: *c1ccc2c(N)ncc(Cl)c2c1] |
| 5443 | 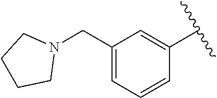 [SMILES: *c1cccc(CN2CCCC2)c1] | N | 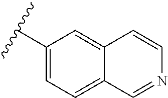 [SMILES: *c1ccc2cnccc2c1] |

TABLE 6

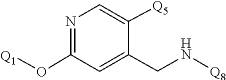

| Example | | | |
|---|---|---|---|
| Number | Q₁ | Q₈ | Q₅ |
| 6442 | 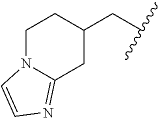 [SMILES: *CC1CCn2ccnc2C1] | 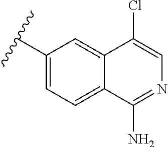 [SMILES: *c1ccc2c(N)ncc(Cl)c2c1] | OiPr |
| 6443 | 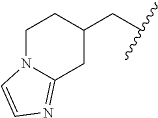 [SMILES: *CC1CCn2ccnc2C1] | 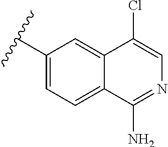 [SMILES: *c1ccc2c(N)ncc(Cl)c2c1] | OMe |
| 6444 | 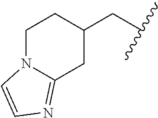 [SMILES: *CC1CCn2ccnc2C1] | 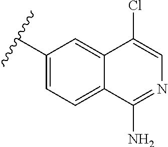 [SMILES: *c1ccc2c(N)ncc(Cl)c2c1] | F |
| 6445 | 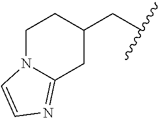 [SMILES: *CC1CCn2ccnc2C1] | 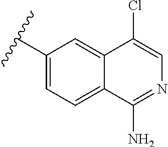 [SMILES: *c1ccc2c(N)ncc(Cl)c2c1] | CF₃ |

TABLE 7

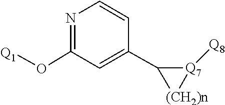

| Example | | | | |
|---|---|---|---|---|
| Number | Q₁ | Q₇ | n | Q₈ |
| 7446 | 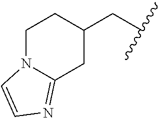 [SMILES: *CC1CCn2ccnc2C1] | CH | 1 | 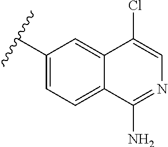 [SMILES: *c1ccc2c(N)ncc(Cl)c2c1] |
| 7447 | 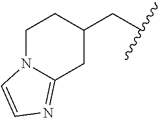 [SMILES: *CC1CCn2ccnc2C1] | CH | 1 | 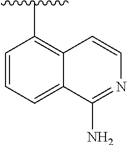 [SMILES: *c1cccc2c(N)nccc12] |
| 7448 | 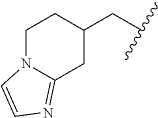 [SMILES: *CC1CCn2ccnc2C1] | CH | 2 | 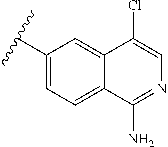 [SMILES: *c1ccc2c(N)ncc(Cl)c2c1] |
| 7449 | 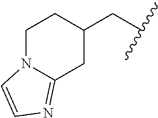 [SMILES: *CC1CCn2ccnc2C1] | CH | 2 | 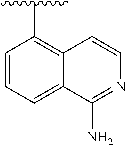 [SMILES: *c1cccc2c(N)nccc12] |
| 7450 | 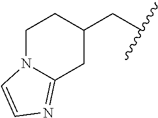 [SMILES: *CC1CCn2ccnc2C1] | N | 2 | 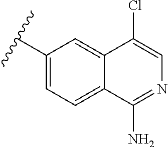 [SMILES: *c1ccc2c(N)ncc(Cl)c2c1] |
| 7451 | 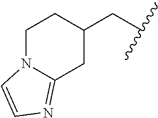 [SMILES: *CC1CCn2ccnc2C1] | N | 2 | 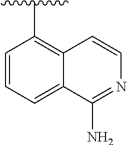 [SMILES: *c1cccc2c(N)nccc12] |
| 7452 | 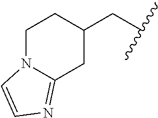 [SMILES: *CC1CCn2ccnc2C1] | N | 3 | 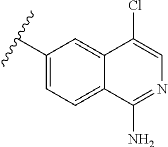 [SMILES: *c1ccc2c(N)ncc(Cl)c2c1] |
| 7453 | 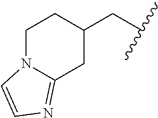 [SMILES: *CC1CCn2ccnc2C1] | N | 3 | 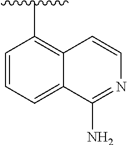 [SMILES: *c1cccc2c(N)nccc12] |

TABLE 8a

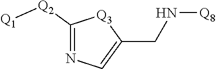

| Example | | | | |
|---|---|---|---|---|
| Number | Q₁ | Q₂ | Q₃ | Q₈ |
| 8454 | 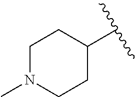 [SMILES: *C1CCN(C)CC1] | O | S | 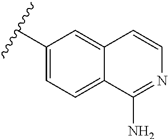 [SMILES: *c1ccc2c(N)nccc2c1] |
| 8455 | 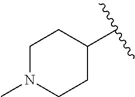 [SMILES: *C1CCN(C)CC1] | OCH₂ | S | 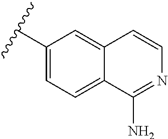 [SMILES: *c1ccc2c(N)nccc2c1] |
| 8456 | 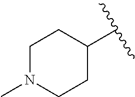 [SMILES: *C1CCN(C)CC1] | O | S | 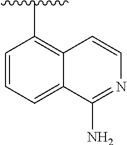 [SMILES: *c1cccc2c(N)nccc12] |
| 8457 | 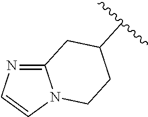 [SMILES: *C1CCn2ccnc2C1] | O | S | 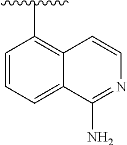 [SMILES: *c1cccc2c(N)nccc12] |

TABLE 8b

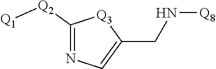

| Example | | | | |
|---|---|---|---|---|
| Number | Q₁ | Q₂ | Q₃ | Q₈ |
| 8458 | 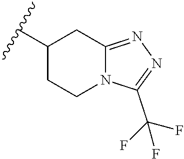 [SMILES: *C1CCn2c(nnc2C(F)(F)F)C1] | Absent | O | 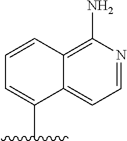 [SMILES: *c1cccc2c(N)nccc12] |

TABLE 8c

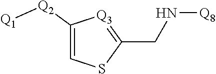

| Example | | | | |
|---|---|---|---|---|
| Number | Q₁ | Q₂ | Q₃ | Q₈ |
| 8459 | 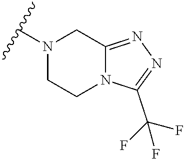 [SMILES: *N1CCn2c(nnc2C(F)(F)F)C1] | Absent | CH | 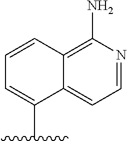 [SMILES: *c1cccc2c(N)nccc12] |

TABLE 9

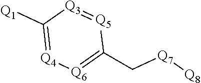

| Example | | | | | | | |
|---|---|---|---|---|---|---|---|
| Number | Q₁ | Q₃ | Q₄ | Q₅ | Q₆ | Q₇ | Q₈ |
| 9001 | 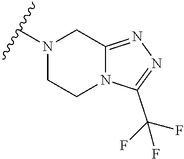 [SMILES: *N1CCn2c(nnc2C(F)(F)F)C1] | CH | CH | CH | N | NH | 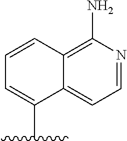 [SMILES: *c1cccc2c(N)nccc12] |
| 9002 | 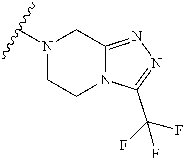 [SMILES: *N1CCn2c(nnc2C(F)(F)F)C1] | CH | N | N | CH | NH | 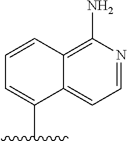 [SMILES: *c1cccc2c(N)nccc12] |
| 9003 | 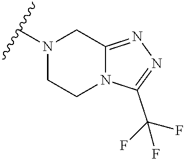 [SMILES: *N1CCn2c(nnc2C(F)(F)F)C1] | N | N | CH | CH | NH | 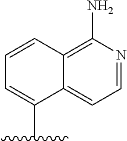 [SMILES: *c1cccc2c(N)nccc12] |
| 9004 | 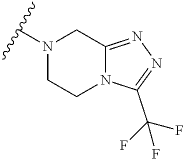 [SMILES: *N1CCn2c(nnc2C(F)(F)F)C1] | N | CH | N | CH | NH | 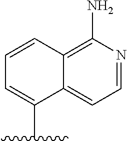 [SMILES: *c1cccc2c(N)nccc12] |
| 9005 | 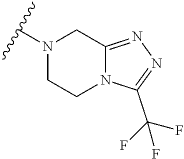 [SMILES: *N1CCn2c(nnc2C(F)(F)F)C1] | CH | CH | CCH₃ | N | NH | 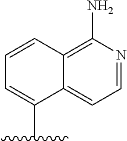 [SMILES: *c1cccc2c(N)nccc12] |
| 9006 | 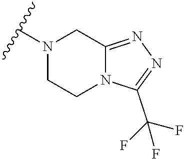 [SMILES: *N1CCn2c(nnc2C(F)(F)F)C1] | N | CH | CCH₃ | CCH₃ | NH | 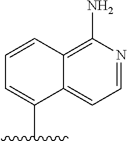 [SMILES: *c1cccc2c(N)nccc12] |
| 9007 | 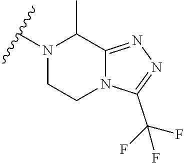 [SMILES: *N1CCn2c(nnc2C(F)(F)F)C1C] | CH | CH | N | CCH₃ | NH | 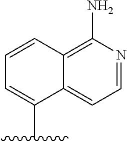 [SMILES: *c1cccc2c(N)nccc12] |
| 9008 | 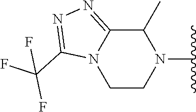 [SMILES: *N1CCn2c(nnc2C(F)(F)F)C1C] | CH | CH | N | CF | NH | 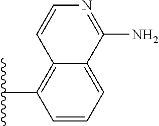 [SMILES: *c1cccc2c(N)nccc12] |
| 1345 | 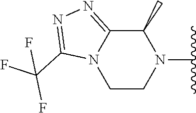 [SMILES: *N1CCn2c(nnc2C(F)(F)F)[C@H]1C] | CH | CH | N | CCH₃ | NH | 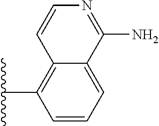 [SMILES: *c1cccc2c(N)nccc12] |
| 1346 | 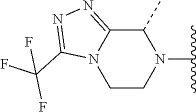 [SMILES: *N1CCn2c(nnc2C(F)(F)F)[C@@H]1C] | CH | CH | N | CCH₃ | NH | 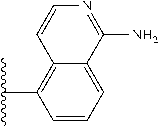 [SMILES: *c1cccc2c(N)nccc12] |

TABLE 10

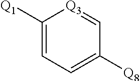

| Example | | | |
|---|---|---|---|
| Number | Q₁ | Q₃ | Q₈ |
| 10001 | 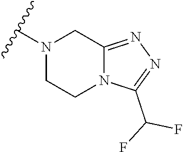 [SMILES: *N1CCn2c(nnc2C(F)F)C1] | N | 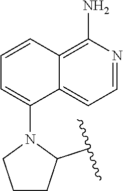 [SMILES: *C1CCCN1c1cccc2c(N)nccc12] |
| 10002 | 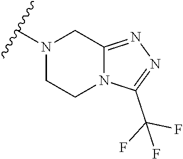 [SMILES: *N1CCn2c(nnc2C(F)(F)F)C1] | N | 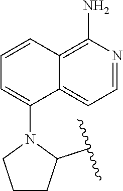 [SMILES: *C1CCCN1c1cccc2c(N)nccc12] |
| 10003 | 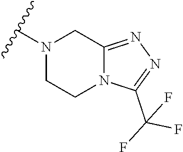 [SMILES: *N1CCn2c(nnc2C(F)(F)F)C1] | N | 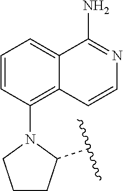 [SMILES: *[C@@H]1CCCN1c1cccc2c(N)nccc12] |
| 10004 | 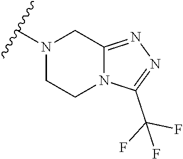 [SMILES: *N1CCn2c(nnc2C(F)(F)F)C1] | N | 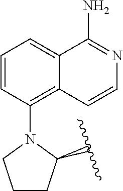 [SMILES: *[C@H]1CCCN1c1cccc2c(N)nccc12] |

Preferably, the compound of formula (I) is a compound selected from example numbers: 1033, 1243, 1251, 1282, 1295, 1299, 1303, 1305, 1309, 1311, 1314, 1316, 1319, 1342, 1344, 1345, 2178, 2197, 2199, 2201, 2256, 4261, 4267, 4268, 4270, 4285, 4298, 4430, 4446, 9005, 9007, 9008, 1002, 1005, 1006, 1009, 1010, 1012, 1013, 1016, 1023, 1024, 1027, 1029, 1042, 1044, 1193, 1195, 1202, 1279, 1300, 1301, 1313, 1321, 1331, 1333, 2177, 2185, 2186, 2191, 2192, 2198, 2202, 2212, 2213, 2216, 2254, 2257, 4260, 4265, 4269, 4277, 4278, 4284, 4297, 4299, 4300, 4303, 4309, 4319, 4320, 4408, 4412, 4414, 4424, 4431, 4434, 4437, 4438, 4439, 4441, 4443, 4444, 4445, 4450, 4467, 8459, 9001, and 9006,
and pharmaceutically acceptable salts and/or solvates thereof.

Preferably, the compound of formula (I) is a compound selected from example numbers: 1033, 2178, 2197, 2199, 2201, 4261, 4267, 4268, 4270, 4285, 4298, 4430, 1002, 1005, 1006, 1009, 1010, 1012, 1013, 1016, 1023, 1024, 1027, 1029, 1042, 1044, 2177, 2185, 2186, 2191, 2192, 2198, 2202, 2212, 2213, 2216, 4260, 4265, 4269, 4277, 4278, 4284, 4297, 4299, 4300, 4303, 4309, 4319, 4320, 4408, 4412, 4414, 4424 and 4431,
and pharmaceutically acceptable salts and/or solvates thereof.

More preferably, the compound of formula (I) is a compound selected from example numbers: 1202, 1096, 1274, 1219, 1278, 1251, 1282, 1299, 1305, 1309, 9005, 1311, 1314, 2256, 4265, 2185, 2186, 2191, 2192, 2177, 1010, 1013, 2197, 4260, 4261, 2199, 2198, 1027, 1029, 4267, 2212, 4298, 4300, 4320, 4319, 4430, 4307 and 4309,
and pharmaceutically acceptable salts and/or solvates thereof.

More preferably, the compound of formula (I) is a compound selected from example numbers: 4265, 2185, 2186, 2191, 2192, 2177, 1010, 1013, 2197, 4260, 4261, 2199, 2198, 1027, 1029, 4267, 2212, 4298, 4300, 4320, 4319, 4430, 4307 and 4309,
and pharmaceutically acceptable salts and/or solvates thereof.

More preferably, the compound of formula (I) is a compound selected from example numbers: 1202, 1096, 1274, 1219, 1278, 1251, 1282, 1299, 1305, 1309, 9005, 1311, 1314, and 2256,
and pharmaceutically acceptable salts and/or solvates thereof.

Even more preferably, the compound of formula (I) is a compound selected from example numbers: 1033, 1243, 1251, 1282, 1295, 1299, 1303, 1305, 1309, 1311, 1314, 1316, 1319, 1342, 1344, 1345, 2178, 2197, 2199, 2201, 2256, 4261, 4267, 4268, 4270, 4285, 4298, 4430, 4446, 9005, 9007, and 9008,
and pharmaceutically acceptable salts and/or solvates thereof.

Even more preferably, the compound of formula (I) is a compound selected from example numbers: 1033, 2178, 2197, 2199, 2201, 4261, 4267, 4268, 4270, 4285, 4298, and 4430,
and pharmaceutically acceptable salts and/or solvates thereof.

Yet more preferably, the compound of formula (I) is a compound selected from example numbers: 1029, 1243, 1274, 1277, 1282, 1305, 2186, 2191, 2197, 2212, 4260, 4268, 4299, and 4301,
and pharmaceutically acceptable salts and/or solvates thereof.

Yet more preferably, the compound of formula (I) is a compound selected from example numbers: 4292, 2186, 2191, 2197, 4260 and 4268,
and pharmaceutically acceptable salts and/or solvates thereof.

Yet more preferably, the compound of formula (I) is a compound selected from example numbers: 1029, 2186, 2191, 2197, 4260 and 4268,
and pharmaceutically acceptable salts and/or solvates thereof.

Therapeutic Applications

As noted above, the compounds (or pharmaceutically acceptable salts and/or solvates thereof), and pharmaceutical compositions comprising the compounds (or pharmaceutically acceptable salts and/or solvates thereof) of the present invention are inhibitors of FXIIa. They are therefore useful in the treatment of disease conditions for which FXIIa is a causative factor.

Accordingly, the present invention provides a compound of the invention (or a pharmaceutically acceptable salt and/or solvate thereof), or a pharmaceutical composition comprising a compound of the invention (or a pharmaceutically acceptable salt and/or solvate thereof), for use in medicine.

The present invention also provides for the use of a compound of the invention (or a pharmaceutically acceptable salt and/or solvate thereof), or a pharmaceutical composition comprising the compound of the invention (or a pharmaceutically acceptable salt and/or solvate thereof), in the manufacture of a medicament for the treatment or prevention of a disease or condition in which FXIIa activity is implicated.

The present invention also provides a method of treatment of a disease or condition in which FXIIa activity is implicated comprising administration to a subject in need thereof a therapeutically effective amount of a compound of the invention (or a pharmaceutically acceptable salt and/or solvate thereof), or a pharmaceutical composition comprising the compound of the invention (or a pharmaceutically acceptable salt and/or solvate thereof).

As discussed above, FXIIa can mediate the conversion of plasma kallikrein from plasma prekallikrein. Plasma kallikrein can then cause the cleavage of high molecular weight kininogen to generate bradykinin, which is a potent inflammatory hormone. Inhibiting FXIIa has the potential to inhibit (or even prevent) plasma kallikrein production. Thus, the disease or condition in which FXIIa activity is implicated can be a bradykinin-mediated angioedema.

The bradykinin-mediated angioedema can be non-hereditary. For example, the non-hereditary bradykinin-mediated angioedema can be selected from non-hereditary angioedema with normal C1 Inhibitor (AE-nC11 nh), which can be environmental, hormonal, or drug-induced; acquired angioedema; anaphylaxis associated angioedema; angiotensin converting enzyme (ACE or ace) inhibitor-induced angioedema; dipeptidyl peptidase-4 inhibitor-induced angioedema; and tPA-induced angioedema (tissue plasminogen activator-induced angioedema).

Alternatively, and preferably, the bradykinin-mediated angioedema can be hereditary angioedema (HAE), which is angioedema caused by an inherited dysfunction/fault/mutation. Types of HAE that can be treated with compounds according to the invention include HAE type 1, HAE type 2, and normal C1 inhibitor HAE (normal C1 nh HAE).

The disease or condition in which FXIIa activity is implicated can be selected from vascular hyperpermeability, stroke including ischemic stroke and haemorrhagic accidents; retinal edema; diabetic retinopathy; DME; retinal vein occlusion; and AMD. These conditions can also be bradykinin-mediated.

As discussed above, FXIIa can activate FXIa to cause a coagulation cascade. Thrombotic disorders are linked to this cascade. Thus, the disease or condition in which FXIIa activity is implicated can be a thrombotic disorder. More specifically, the thrombotic disorder can be thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; acute respiratory distress syndrome (ARDS); idiopathic pulmonary fibrosis (IPF); rheumatoid arthritis (RA); and cold-induced urticarial autoinflammatory syndrome.

Surfaces of medical devices that come into contact with blood can cause thrombosis. The compounds (or pharmaceutically acceptable salts and/or solvates thereof) and pharmaceutical compositions of the present invention can be coated on the surfaces of devices that come into contact with blood to mitigate the risk of the device causing thrombosis. For instance, they can lower the propensity these devices to clot blood and therefore cause thrombosis. Examples of devices that come into contact with blood include vascular grafts, stents, in dwelling catheters, external catheters, orthopedic prosthesis, cardiac prosthesis, and extracorporeal circulation systems.

Other disease conditions for which FXIIa is a causative factor include: neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

Combination Therapy

The compounds of the present invention (or pharmaceutically acceptable salts and/or solvates thereof) may be administered in combination with other therapeutic agents. Suitable combination therapies include any compound of the present invention (or a pharmaceutically acceptable salt and/or solvate thereof) combined with one or more agents selected from agents that inhibit platelet-derived growth factor (PDGF), endothelial growth factor (VEGF), integrin alpha5beta1, steroids, other agents that inhibit FXIIa and other inhibitors of inflammation.

Some specific examples of therapeutic agents that may be combined with the compounds of the present invention include those disclosed in EP2281885A1 and by S. Patel in Retina, 2009 June; 29(6 Suppl):S45-8.

Other suitable combination therapies include a compound of the invention (or a pharmaceutically acceptable salt and/or solvate thereof) combined with one or more agents selected from agents that treat HAE (as defined generally herein), for example bradykinin B2 antagonists such icatibant (Firazyr®); plasma kallikrein inhibitors such as ecallantide (Kalbitor®), lanadelumab (Takhzyro®) and berotralstat (ORLADEYO™); or C1 esterase inhibitor such as Cinryze® and Haegarda® and Berinert® and Ruconest®.

Other suitable combination therapies include a compound of the invention (or a pharmaceutically acceptable salt and/or solvate thereof) combined with one or more agents selected from agents that are antithrombotics (as outlined above), for example other Factor XIIa inhibitors, thrombin receptor antagonists, thrombin inhibitors, factor Vila inhibitors, factor Xa inhibitors, factor XIa inhibitors, factor IXa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis) and aspirin) and platelet aggregation inhibitors.

When combination therapy is employed, the compounds of the present invention and said combination agents may exist in the same or different pharmaceutical compositions, and may be administered separately, sequentially or simultaneously.

The compounds of the present invention can be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema” Ophthalmology. 27 Apr. 2010).

Intermediates

Another aspect of the invention provides a compound of formula (II), which are intermediates in the synthesis of the compounds of formula (I):

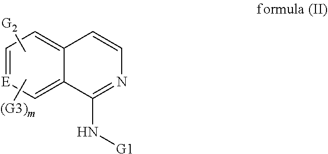

wherein:
E is selected from CH and N;
G1 is either:

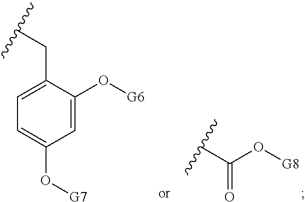

G2 is F, Cl, or Br;
m is 0, 1 or 2;
G3, when present, is independently selected from alkyl, OH, OCF₃, aryl^{b}, heteroaryl^{b}, alkoxy, CF₃, CN, —(CH₂)₀₋₃—N(G4)(G5), —C(═O)OR12, —C(═O)NR13R14 and halo; provided that when m is 1, G3 is not methyl;
G4 and G5 are independently selected from alkyl^{b}, aryl^{b} and heteroaryl^{b} or G4 and G5 together with the nitrogen atom to which they are attached form a carbon-containing 4-, 5-, 6- or 7-membered heterocyclic ring, optionally containing an additional heteroatom selected from N, NR12, S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
G6 and G7 are independently selected from methyl, ethyl, n-propyl and i-propyl;
G8 is selected from methyl, ethyl, n-propyl, i-propyl, n-butyl and i-butyl;
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from (C₁-C₆)alkoxy, OH, —NR13R14, —C(═O)OR13, —C(═O)NR13R14, CN, CF₃, halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from (C₁-C₆)alkoxy, OH, CN, CF₃, halo;
aryl^{b} is phenyl, biphenyl or naphthyl; aryl^{b} may be optionally substituted with 1, 2 or 3 substituents independently selected from alkyl^{b}, alkoxy, OH, halo, CN, and CF₃;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, halo;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, and fluoro;
halo is F, Cl, Br, or I;
heteroaryl is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃;
heteroaryl^{a} is a 5, 6, 9 or 10 membered mono- or bi-cyclic aromatic ring, containing, where possible, 1, 2, 3 or 4 ring members independently selected from N, NR12, S and O; heteroaryl^{a} may be optionally substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃;
heteroaryl^{b} is a 5, 6, 9 or 10 membered mono- or bi-cyclic aromatic ring, containing, where possible, 1, 2 or 3 ring members independently selected from N, NR12, S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃;
heterocycloalkyl is a non-aromatic carbon-containing monocyclic ring containing 5, 6, or 7 ring members, wherein one or two ring members are independently selected from N, NR8, S, SO, SO₂, and O; wherein heterocycloalkyl may be optionally substituted with 1, 2, or 3 substituents independently selected from alkyl, alkoxy, OH, OCF₃, halo, oxo and CN;
R8 is independently selected from H, alkyl, cycloalkyl, or heterocycloalkyl^{a};
heterocycloalkyl^{a} is a non-aromatic carbon-containing monocyclic ring containing 3, 4, 5, or 6, ring members, wherein at least one ring member is independently selected from N, NR12, S, and O; heterocycloalkyl^{a} may be optionally be substituted with 1 or 2 substituents independently selected from alkyl, (C₁-Cᵣ)alkoxy, OH, CN, CF₃, halo;
R12 is independently selected from H, alkyl, or cycloalkyl;
R13 and R14 are independently selected from H, alkyl^{b}, aryl^{b} and heteroaryl^{b} or R13 and R14 together with the nitrogen atom to which they are attached form a carbon-containing 4-, 5-, 6- or 7-membered heterocyclic ring, optionally containing an additional heteroatom selected from N, NR12, S, SO, SO₂, and O, which may be saturated or unsaturated with 1 or 2 double bonds and which may be optionally mono- or di-substituted with substituents selected from oxo, alkyl^{b}, alkoxy, OH, halo and CF₃; and tautomers, isomers, stereoisomers (including enantiomers, diastereoisomers and racemic and scalemic mixtures thereof), deuterated isotopes, and salts and/or solvates thereof.

It will be understood that “salts and/or solvates thereof” means “salts thereof”, “solvates thereof”, and “solvates of salts thereof”.

Preferably, when m is 0; G2 is substituted at any ring member apart from the ring member marked **

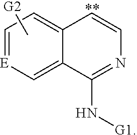

In this instance, it will be understood that, when m is 0; G2 is substituted at any ring member apart from the ring member marked **

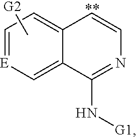

i.e. G2 may be substituted, where possible, at any of the following ring members:

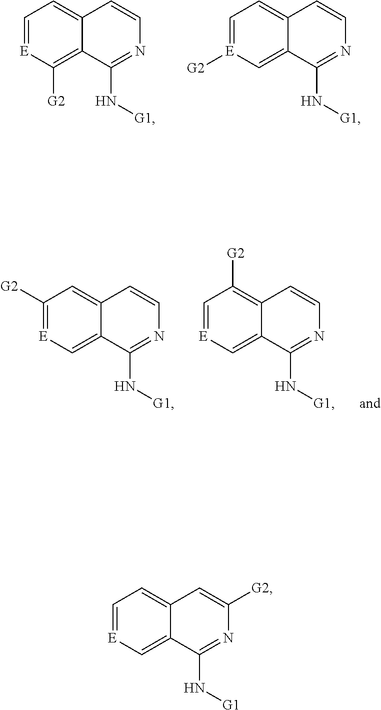

but not at the following ring member:

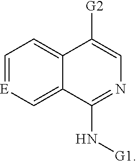

Preferably, G8 is selected from methyl, n-propyl, i-propyl, n-butyl and i-butyl.
G2 can be selected from C1 and Br. G2 can be C1. G2 can be Br.
m can be 0 or 1. m can be 1. m can be 0.
G3 can be selected from alkyl, alkoxy, OH, OCF₃, halo, CN, and CF₃. Preferably G3 is halo. When G3 is halo,
G3 can be selected from Cl and F. G3 can be C1. G3 can be F.
E can be CH. E can be N.

Preferably G1 is selected from

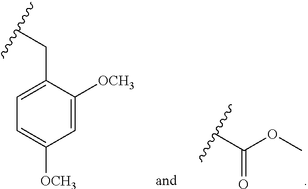

### G1 can be

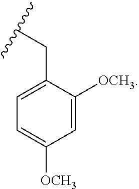 [SMILES: *Cc1ccc(OC)cc1OC]

### G1 can be

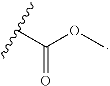 [SMILES: *C(=O)OC]

Preferably, the compound of formula (II) is selected from

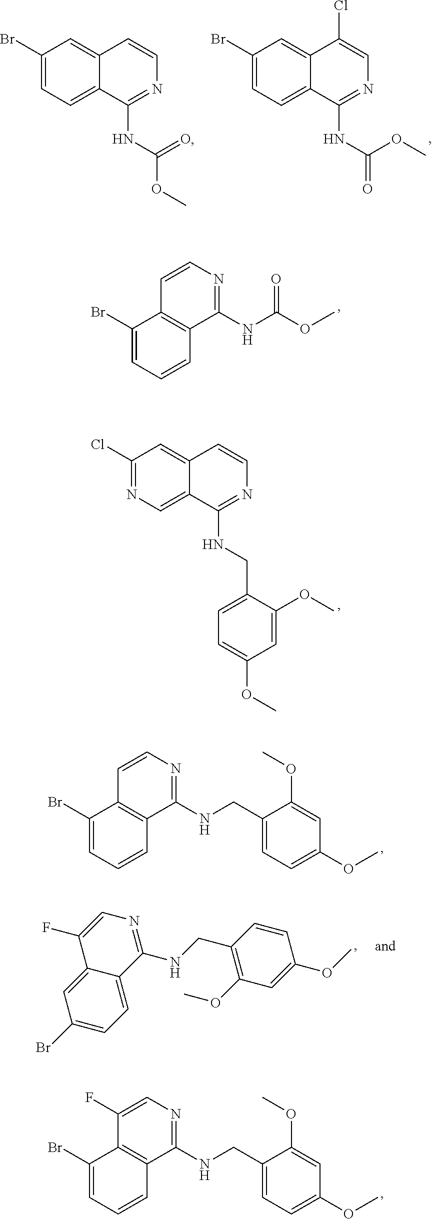

or a salt, solvate, or a solvate of a salt thereof.

### Definitions

As noted above, the term “alkyl” is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from (C₁-C₆)alkoxy, OH, —NR13R14, —C(═O)OR13, —C(═O)NR13R14, CN, CF₃, halo. As noted above “alkyl^{b}”, is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from (C₁-C₆)alkoxy, OH, CN, CF₃, halo. Examples of such alkyl or alkyl^{b} groups include, but are not limited, to C1-methyl, C₂-ethyl, C₃-propyl and C₄-n-butyl, C₃-iso-propyl, C₄-sec-butyl, C₄-iso-butyl, C₄-tert-butyl and C₅-neo-pentyl, optionally substituted as noted above. More specifically, “alkyl” or “alkyl^{b}” can be a linear saturated hydrocarbon having up to 6 carbon atoms (C₁-C₆) or a branched saturated hydrocarbon of between 3 and 6 carbon atoms (C₃-C₅), optionally substituted as noted above. Even more specifically, “alkyl” or “alkyl^{b}”, can be a linear saturated hydrocarbon having up to 4 carbon atoms (C₁-C₄) or a branched saturated hydrocarbon of between 3 and 4 carbon atoms (C₃-C₄), optionally substituted as noted above, which is herein called “small alkyl” or “small alkyl^{b},”, respectively. Preferably, “alkyl” or “alkyl^{b}”, can be defined as a “small alkyl” or “small alkyl^{b}”.

As noted above, the term “alkylene” is a bivalent linear saturated hydrocarbon having 1 to 5 carbon atoms (C₁-C₅); alkylene may optionally be substituted with 1 or 2 substituents independently selected from alkyl^{b}, (C₁-C₆)alkoxy, OH, CN, CF₃, halo. More specifically, “alkylene” can be a bivalent linear saturated hydrocarbon having 2 to 4 carbon atoms (C₂-C₄), more specifically having 2 to 3 carbon atoms (C₂-C₃), optionally substituted as noted above.

“Aryl” and “aryl^{b}” are as defined above. Typically, “aryl” or “aryl^{b}”, will be optionally substituted with 1, 2 or 3 substituents. Optional substituents are selected from those stated above. Examples of suitable aryl or aryl^{b} groups include phenyl, biphenyl and naphthyl (each optionally substituted as stated above). Preferably “aryl” is selected from phenyl, substituted phenyl (wherein said substituents are selected from those stated above) and naphthyl. Most preferably “aryl” is selected from phenyl and substituted phenyl (wherein said substituents are selected from those stated above).

As noted above, the term “cycloalkyl” is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, halo. Examples of suitable monocyclic cycloalkyl groups include cyclopropyl, cyclobutyl, cyclopentyl, and cyclohexyl, optionally substituted as noted above. More specifically, “cycloalkyl” can be a monocyclic saturated hydrocarbon ring of between 3 and 5 carbon atoms, more specifically, between 3 and 4 carbon atoms, optionally substituted as noted above.

As noted above, the term “alkoxy” is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, and fluoro. Examples of such alkoxy groups include, but are not limited to, C1-methoxy, C₂-ethoxy, C₃-n-propoxy and C₄-n-butoxy for linear alkoxy, and C₃-iso-propoxy, and C₄-sec-butoxy and tert-butoxy for branched alkoxy, optionally substituted as noted aboves. More specifically, “alkoxy” can be linear groups of between 1 and 4 carbon atoms (C₁-C₄), more specifically, between 1 and 3 carbon atoms (C₁-C₃). More specifically, “alkoxy” can be branched groups of between 3 and 4 carbon atoms (C₃-C₄), optionally substituted as noted above.

“Halo” can be selected from Cl, F, Br and I. More specifically, halo can be selected from Cl and F.

As noted above, “heteroaryl” is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃. For example, heteroaryl can be selected from thiophene, furan, pyrrole, pyrazole, imidazole, oxazole, isoxazole, thiazole, isothiazole, triazole, oxadiazole, thiadiazole, pyridine, pyridazine, pyrimidine, and pyrazine, optionally substituted as noted above.

“Heteroaryl^{a}” and “heteroaryl^{b}” are as defined above. Typically, “heteroaryl^{a}” or “heteroaryl^{b}” will be optionally substituted with 1, 2 or 3 substituents. Optional substituents are selected from those stated above. Examples of suitable heteroaryl^{a} or heteroaryl^{b} groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl, isoquinolinyl, 5-azathianaphthenyl, indolizinyl, isoindolyl, azaindolyl, indazolyl, benzothiazolyl, cinnolinyl, quinazolinyl, quinoxalinyl, 1,8-napthyridinyl and phthalazinyl (optionally substituted as stated above). Examples of suitable heteroaryl^{a} or heteroaryl^{b} groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl, isoquinolinyl, 5-azathianaphthenyl, indolizinyl, isoindolyl, indazolyl, benzothiazolyl, cinnolinyl, quinazolinyl, quinoxalinyl, 1,8-napthyridinyl and phthalazinyl (optionally substituted as stated above). More specifically, “heteroaryl^{a}” or “heteroaryl^{b}” can be a 9- or 10-membered bi-cyclic ring as defined, and optionally substituted as stated above. Examples of suitable 9- or 10-membered heteroaryl^{a} or heteroaryl^{b} groups include indolyl, benzimidazolyl, benzotriazolyl, quinolinyl, isoquinolinyl, 5-azathianaphthenyl, indolizinyl, isoindolyl, azaindolyl, indazolyl, benzothiazolyl, cinnolinyl, quinazolinyl, quinoxalinyl, 1,8-napthyridinyl and phthalazinyl. Examples of suitable 9- or 10-membered heteroaryl^{a} or heteroaryl^{b} groups include indolyl, benzimidazolyl, benzotriazolyl, quinolinyl, isoquinolinyl, 5-azathianaphthenyl, indolizinyl, isoindolyl, indazolyl, benzothiazolyl, cinnolinyl, quinazolinyl, quinoxalinyl, 1,8-napthyridinyl and phthalazinyl.

Preferably, heteroaryl^{b} is heteroaryl^{c}. Heteroaryl^{c} is a 5, 6, 9 or 10 membered mono- or bi-cyclic aromatic ring, containing, where possible, 1 or 2 ring members independently selected from N, NR12, S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃.

As noted above, “heterocycloalkyl” is a non-aromatic carbon-containing monocyclic ring containing 5, 6, or 7 ring members, wherein one or two ring members are independently selected from N, NR8, S, SO, SO₂, and O; wherein heterocycloalkyl may be optionally substituted with 1, 2, or 3 substituents independently selected from alkyl, alkoxy, OH, OCF₃, halo, oxo and CN. More specifically, “heterocycloalkyl” can be a non-aromatic carbon-containing monocyclic ring containing 5, 6, or 7 ring members, wherein one or two ring members are independently selected from N, NR8, and O, optionally substituted as noted above. More specifically, “heterocycloalkyl” can be a non-aromatic carbon-containing monocyclic ring containing 5, 6, or 7 ring members, wherein one or two ring members are independently selected from N or NR8.

As noted above, “heterocycloalkyl^{a}” is a non-aromatic carbon-containing monocyclic ring containing 3, 4, 5, or 6, ring members, wherein at least one ring member is independently selected from N, NR12, S, and O; heterocycloalkyl^{a} may be optionally be substituted with 1 or 2 substituents independently selected from alkyl, (C₁-C₆)alkoxy, OH, CN, CF₃, halo. More specifically, “heterocycloalkyl^{a}” can be a non-aromatic carbon-containing monocyclic ring containing 3, 4, 5, or 6, ring members, wherein at least one ring member is independently selected from NR12, and O; heterocycloalkyl^{a} may be optionally substituted with 1 or 2 substituents independently selected from alkyl (C₁-C₆)alkoxy, OH, CN, CF₃, halo.

The term “O-linked”, such as in “O-linked hydrocarbon residue”, means that the hydrocarbon residue is joined to the remainder of the molecule via an oxygen atom.

The term “N-linked”, such as in “N-linked pyrrolidinyl”, means that the heterocycloalkyl group is joined to the remainder of the molecule via a ring nitrogen atom.

In groups such as —(CH₂)₀₋₆-A, “-” denotes the point of attachment of the substituent group to the remainder of the molecule.

As is clear from the definitions above, and for the avoidance of any doubt, it will be understood that “Y” is defined above, and does not encompass Yttrium.

As is clear from the definitions above, and for the avoidance of any doubt, it will be understood that “B” is defined above, and does not encompass Boron.

As is clear from the definitions above, and for the avoidance of any doubt, it will be understood that “W” is defined above, and does not encompass Tungsten.

“Salt”, as used herein (including “pharmaceutically acceptable salt”) means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, base addition salts (including pharmaceutically acceptable base addition salts) that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, acid addition salts (including pharmaceutically acceptable acid addition salts) that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates, trifluoroacetates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002). “Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2^{nd} Ed. pp 561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometric, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis). For example, where compounds of the invention exist as a mixture of stereoisomers, one stereoisomer can be present at a purity of >90% relative to the remaining stereoisomers, or more specifically at a purity of >95% relative to the remaining stereoisomers, or yet more specifically at a purity of >99% relative to the remaining stereoisomers. For example, where compounds of the invention exists in enantiomeric forms, the compound can be >90% enantiomeric excess (ee), or more specifically >95% enantiomeric excess (ee), or yet more specifically, >99% ee.

Unless otherwise stated, the compounds of the invention include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention. Such compounds are useful, for example, as analytical tools or probes in biological assays.

In the context of the present invention, references herein to “treatment” include references to curative, palliative and prophylactic treatment. For instance, treatment includes preventing the symptoms of the disease conditions for which FXIIa is a causative factor.

Methods

The compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of the invention and a pharmaceutically acceptable carrier, diluent or excipient.

For the treatment of conditions such as retinal vascular permeability associated with diabetic retinopathy and diabetic macular edema, the compounds of the invention may be administered in a form suitable for injection into the ocular region of a patient, in particular, in a form suitable for intra-vitreal injection. It is envisaged that formulations suitable for such use will take the form of sterile solutions of a compound of the invention in a suitable aqueous vehicle. The compositions may be administered to the patient under the supervision of the attending physician.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

Preferably, the compounds of the invention are administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solids and liquids (including multiple phases or dispersed systems). Exemplary formulations suitable for oral administration include tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

For administration to human patients, the total daily dose of the compounds of the invention is typically in the range 0.1 mg and 10,000 mg, or between 1 mg and 5000 mg, or between 10 mg and 1000 mg depending, of course, on the mode of administration.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

### Numbered Embodiments

The invention is also described by the following numbered embodiments:

1. A compound of formula (I),

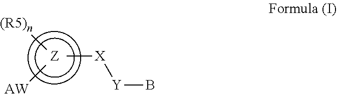

wherein:
Z is a 6- or 5-membered heteroaromatic ring containing 1, 2 or 3 ring members independently selected from N, S and O; or phenyl; or,
Z is 2-pyridone or 4-pyridone,
X is selected from SO₂ and CR1R2;
R1 is selected from H, alkyl, alkoxy, OH, halo and NR13R14; and
R2 is selected from H and small alkyl; or
R1 and R2, together with the carbon atom to which they are attached, are linked by alkylene to form a 3-, 4-, or 5-membered saturated ring;
Y is selected from NR12, 0, and CR3R4;
R3 and R4 are independently selected from H and alkyl; or
X is CR1R2 and Y is CR3R4, and R1 and R3, together with the carbon atom to which R1 is attached and the carbon atom to which R3 is attached, are linked by alkylene to form a 3-, 4-, or 5-membered saturated ring; or
X is CR1R2 and Y is NR12, and R1 and R12, together with the carbon atom to which R1 is attached and the nitrogen atom to which R12 is attached, are linked by alkylene to form a 3-, 4-, or 5-membered saturated heterocycle;
B is selected from:
(i) heteroaryl^{a};
(ii) aryl;
(iii) a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃; and
(iv) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring;
n is 0, 1 or 2;
when present, each R5 is independently selected from alkyl, cyclopropyl, alkoxy, halo, OH, CN, (CH₂)₀₋₆COOH, and CF₃;
AW- is selected from:
—(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—NR12-(CH₂)₁₋₆—C(═O)-A, —(CH₂)₀₋₆—NH—C(═O)—(CH₂)₀₋₆-A, —C(═O)NR12-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A and —SO₂—NH-A;
A is a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN;
wherein when A is a bicyclic ring system, the bicyclic ring system is fused, bridged or spiro; wherein when A is a tricyclic ring system, each of the three rings in the tricyclic ring system is either fused, bridged or spiro to at least one of the other rings in the tricyclic ring system;
alkyl is a linear saturated hydrocarbon having up to 10 carbon atoms (C₁-C₁₀) or a branched saturated hydrocarbon of between 3 and 10 carbon atoms (C₃-C₂₀); alkyl may optionally be substituted with 1, 2 or 3 substituents independently selected from (C₁-C₆)alkoxy, OH, —NR13R14, —C(═O)OR13, —C(═O)NR13R14, CN, CF₃, halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from (C₁-C₆)alkoxy, OH, CN, CF₃, halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from (C1-C6)alkoxy, OH, NR13R14, C(═O)OR13, C(═O)NR13R14, CN, CF3, halo;
small alkyl^{b} is linear saturated hydrocarbon having up to 4 carbon atoms (C₁-C₄) or a branched saturated hydrocarbon of between 3 and 4 carbon atoms (C₃-C₄); small alkyl^{b} may optionally be substituted with 1 or 2 substituents independently selected from (C₁-C₆)alkoxy, OH, CN, CF₃, halo;
alkylene is a bivalent linear saturated hydrocarbon having 1 to 5 carbon atoms (C₁-C₅); alkylene may optionally be substituted with 1 or 2 substituents independently selected from alkyl^{b}, (C₁-C₆)alkoxy, OH, CN, CF₃, halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ and CF₃;
aryl^{b} is phenyl, biphenyl or naphthyl; aryl^{b} may be optionally substituted with 1, 2 or 3 substituents independently selected from alkyl^{b}, alkoxy, OH, halo, CN, and CF₃;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, halo;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, and fluoro;
halo is F, Cl, Br, or I; heteroaryl is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃;
heteroaryl^{a} is a 5, 6, 9 or 10 membered mono- or bi-cyclic aromatic ring, containing, where possible, 1, 2, 3 or 4 ring members independently selected from N, NR12, S and O; heteroaryl^{a} may be optionally substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃;
heteroaryl^{b} is a 5, 6, 9 or 10 membered mono- or bi-cyclic aromatic ring, containing, where possible, 1, 2 or 3 ring members independently selected from N, NR12, S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃;
heterocycloalkyl is a non-aromatic carbon-containing monocyclic ring containing 5, 6, or 7 ring members, wherein one or two ring members are independently selected from N, NR8, S, SO, SO₂, and O; wherein heterocycloalkyl may be optionally substituted with 1, 2, or 3 substituents independently selected from alkyl, alkoxy, OH, OCF₃, halo, oxo and CN;
R8 is independently selected from H, alkyl, cycloalkyl, or heterocycloalkyl^{a}; heterocycloalkyl^{a} is a non-aromatic carbon-containing monocyclic ring containing 3, 4, 5, or 6, ring members, wherein at least one ring member is independently selected from N, NR12, S, and O; heterocycloalkyl^{a} may be optionally be substituted with 1 or 2 substituents independently selected from alkyl, (C₁-C₆)alkoxy, OH, CN, CF₃, halo;
R12 is independently selected from H, alkyl, or cycloalkyl;
R13 and R14 are independently selected from H, alkyl^{b}, aryl_{b} and heteroaryl^{b} or R13 and R14 together with the nitrogen atom to which they are attached form a carbon-containing 4-, 5-, 6- or 7-membered heterocyclic ring, optionally containing an additional heteroatom selected from N, NR12, S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
and tautomers, isomers, stereoisomers (including enantiomers, diastereoisomers and racemic and scalemic mixtures thereof), deuterated isotopes, and pharmaceutically acceptable salts and/or solvates thereof.

2. A compound of formula (I) according to numbered embodiment 1 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein when AW- is —C(═O)NR12-(CH₂)₀₋₆-A, or —(CH₂)₀₋₆—C(═O)—(CH₂)₀₋₆-A, AW- is bonded at a carbon ring member of Z.

3. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is a 6- or 5-membered heteroaromatic ring containing 1, 2 or 3 ring members independently selected from N, S and O; or phenyl.

4. A compound of formula (I) according to numbered embodiment 3 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is a 6- or 56-membered heteroaromatic ring containing 1 or 2 ring members independently selected from N and S; or phenyl.

5. A compound of formula (I) according to any of numbered embodiments 1 to 2 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is a 6-membered heteroaromatic ring containing 1, 2, or 3 ring members independently selected from N; or phenyl; or, Z is 2-pyridone or 4-pyridone.

6. A compound of formula (I) according to any of numbered embodiments 1 to 3, or 5 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is a 6-membered heteroaromatic ring containing 1, 2, or 3 ring members independently selected from N.

7. A compound of formula (I) according to any of numbered embodiments 1 to 3 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is selected from pyrazole, phenyl, pyrimidine, pyridine, pyrazine, pyridazine, oxazole, thiophene, and thiazole.

8. A compound of formula (I) according to numbered embodiment 7 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is selected from pyrazole, phenyl, pyrimidine, pyridine and thiazole.

9. A compound of formula (I) according to numbered embodiment 8 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is selected from phenyl, pyrimidine and pyridine.

10. A compound of formula (I) according to any of numbered embodiments 1 to 4, or 7 to 9 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is phenyl.

11. A compound of formula (I) according to any of numbered embodiments 1 to 3 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is a 5-membered heteroaromatic ring containing 1, 2 or 3 ring members independently selected from N, S and O.

12. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the compound is selected from:

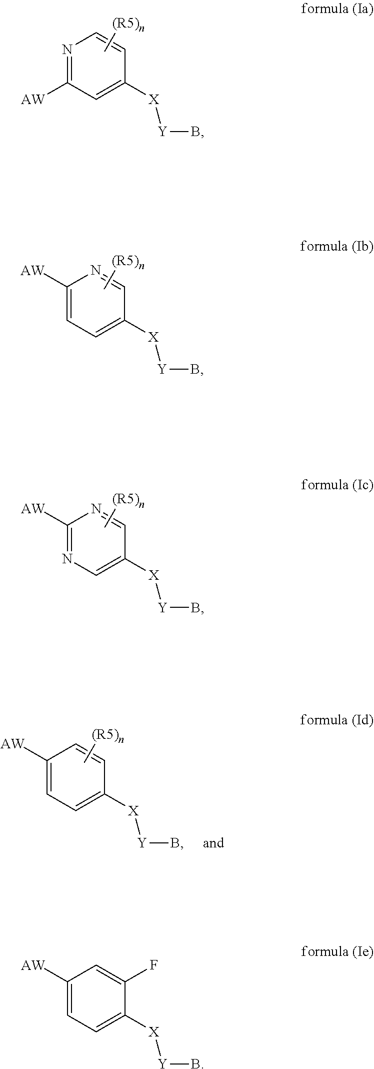

13. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is CR1R2.

14. A compound of formula (I) according to numbered embodiment 13 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is CH₂.

15. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is NR12.

16. A compound of formula (I) according to numbered embodiment 15 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is NH.

17. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
(i) heteroaryl^{a};
(ii) a 5- to 6-membered non-aromatic heterocyclic ring containing one N ring member, which, where possible, may be saturated or unsaturated with 1 or 2 double bonds,
wherein the non-aromatic heterocyclic ring is optionally substituted by 1, 2 or 3 substituents independently selected from alkyl, alkoxy, aryl^{b}, OH, OCF₃, halo, oxo, CN, and CF₃; and
(iii) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring.

18. A compound of formula (I) according to any of numbered embodiments 1-16 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
(i) heteroaryl^{a};
(ii) aryl; and
(iii) a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃.

19. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
(i) heteroaryl^{a}; and
(ii) a 5- to 6-membered non-aromatic heterocyclic ring containing one N ring member, which, where possible, may be saturated or unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃.

20. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is heteroaryl^{a}.

21. A compound of formula (I) according to numbered embodiment 20 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is attached to B at a carbon atom on the heteroaryl^{a} ring.

22. A compound of formula (I) according to numbered embodiment 21 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is attached to B at a carbon atom on the heteroaryl^{a} ring, and the two ring atoms adjacent to the carbon atom on the heteroaryl^{a} ring to which Y attaches are both carbon.

23. A compound of formula (I) according to any of numbered embodiments 20 to 22 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from: isoquinolinyl

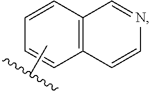

optionally substituted as for heteroaryl^{a}; 6-azaindolyl

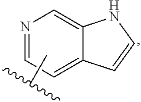

optionally substituted as for heteroaryl^{a}; 7-azaindolyl

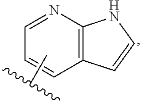

optionally substituted as for heteroaryl^{a}; and pyridyl

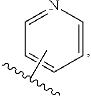

optionally substituted as for heteroaryl^{a}.

24. A compound of formula (I) according to numbered embodiment 23 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, selected from

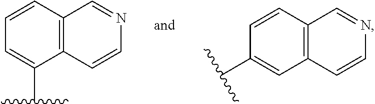 [SMILES: *c1cccc2cnccc12 ; "and" ; *c1ccc2cnccc2c1]

optionally substituted as for heteroaryl^{a};
6-azaindolyl

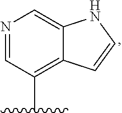 [SMILES: *c1cncc2[nH]ccc12]

optionally substituted as for heteroaryl^{a};
7-azaindolyl

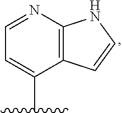 [SMILES: *c1ccnc2[nH]ccc12]

, optionally substituted as for heteroaryl^{a}; and pyridyl

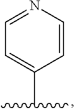 [SMILES: *c1ccncc1]

optionally substituted as for heteroaryl^{a}.

25. A compound of formula (I) according to any of numbered embodiments 20 to 23 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from: isoquinolinyl

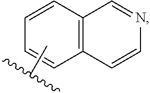

substituted with NH₂, optionally further substituted with 1 or 2 substituents as for heteroaryl^{a};
6-azaindolyl

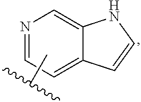

optionally substituted as for heteroaryl^{a};
7-azaindolyl

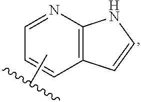

optionally substituted as for heteroaryl^{a}; and
pyridyl

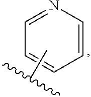

optionally substituted as for heteroaryl^{a}.

26. A compound of formula (I) according to numbered embodiments 25 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from: isoquinolinyl, selected from

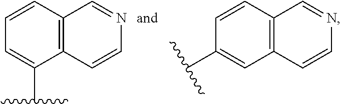 [SMILES: *c1cccc2cnccc12 ; *c1ccc2cnccc2c1]

substituted with NH₂, optionally further substituted with 1 or 2 substituents as for heteroaryl^{a};
6-azaindolyl

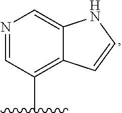 [SMILES: *c1cncc2[nH]ccc12]

optionally substituted as for heteroaryl^{a};
7-azaindolyl

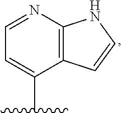 [SMILES: *c1ccnc2[nH]ccc12]

optionally substituted as for heteroaryl^{a}; and
pyridyl

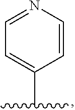 [SMILES: *c1ccncc1]

optionally substituted as for heteroaryl^{a}.

27. A compound of formula (I) according to any of numbered embodiments 20 to 23, or 25 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, substituted with NH₂ at the 1-position

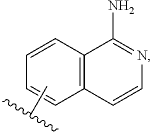

optionally further substituted with 1 or 2 substituents as for heteroaryl^{a};
6-azaindolyl

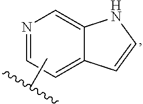

optionally substituted as for heteroaryl^{a};
7-azaindolyl

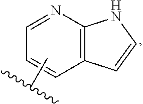

optionally substituted as for heteroaryl^{a}; and
pyridyl

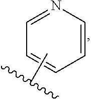

optionally substituted as for heteroaryl^{a}.

28. A compound of formula (I) according to numbered embodiment 27 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, substituted with NH₂ at the 1-position, selected from

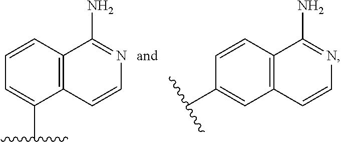 [SMILES: *c1cccc2c(N)nccc12 ; *c1ccc2c(N)nccc2c1]

optionally further substituted with 1 or 2 substituents as for heteroaryl^{a};
6-azaindolyl

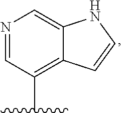 [SMILES: *c1cncc2[nH]ccc12]

optionally substituted as for heteroaryl^{a};
7-azaindolyl

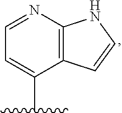 [SMILES: *c1ccnc2[nH]ccc12]

optionally substituted as for heteroaryl^{a}; and
pyridyl

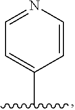 [SMILES: *c1ccncc1]

optionally substituted as for heteroaryl^{a}.

29. A compound of formula (I) according to numbered embodiment 28 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:
isoquinolinyl, substituted with NH₂ at the 1-position, selected from and

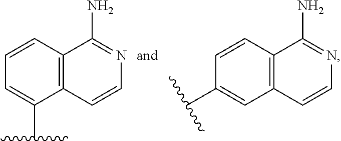 [SMILES: *c1cccc2c(N)nccc12 ; *c1ccc2c(N)nccc2c1]

optionally further substituted with 1 or 2 substituents as for heteroaryl^{a}.

30. A compound of formula (I) according to numbered embodiment 29 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:
isoquinolinyl, substituted with NH₂ at the 1-position

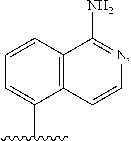 [SMILES: *c1cccc2c(N)nccc12]

optionally further substituted with 1 or 2 substituents as for heteroaryl^{a}.

31. A compound of formula (I) according to numbered embodiment 29 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:
isoquinolinyl, substituted with NH₂ at the 1-position

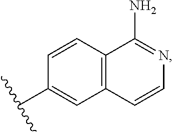 [SMILES: *c1ccc2c(N)nccc2c1]

optionally further substituted with 1 or 2 substituents as for heteroaryl^{a}.

32. A compound of formula (I) according to numbered embodiment 28 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:
6-azaindolyl

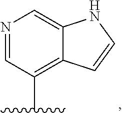 [SMILES: *c1cncc2[nH]ccc12]

optionally substituted as for heteroaryl^{a}.

33. A compound of formula (I) according to numbered embodiment 28 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:
7-azaindolyl

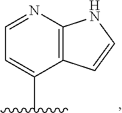 [SMILES: *c1ccnc2[nH]ccc12]

optionally substituted as for heteroaryl^{a}.

34. A compound of formula (I) according to numbered embodiment 28 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:
pyridyl

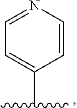 [SMILES: *c1ccncc1]

optionally substituted as for heteroaryl^{a}.

35. A compound of formula (I) according to any of numbered embodiments 20 to 22 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, optionally substituted as for heteroaryl^{a}.

36. A compound of formula (I) according to numbered embodiment 35 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, substituted with 1, 2 or 3 substituents independently selected from alkyl, alkoxy, OH, OCF₃, halo, CN, aryl^{b}, —(CH₂)₁₋₃—NR13R14, heteroaryl^{b}, —C(═O)OR12, —C(═O)NR13R14 and CF₃.

37. A compound of formula (I) according to numbered embodiment 36 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, substituted with 1, 2 or 3 substituents independently selected from alkoxy.

38. A compound of formula (I) according to numbered embodiment 37 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, substituted with 1, 2 or 3 substituents selected from —OMe.

39. A compound of formula (I) according to numbered embodiment 38 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, substituted with NH₂, and optionally substituted with 1 or 2 further substituents as for heteroaryl^{a}.

40. A compound of formula (I) according to any of numbered embodiments 20 to 22 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is a 5, 6, 9 or 10 membered mono- or bi-cyclic aromatic ring, containing, where possible, 1, 2, 3 or 4 ring members independently selected from N, NR12, S and O; which may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃.

41. A compound of formula (I) according to any of numbered embodiments 20 to 40 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the optional substituents on B are, where possible, independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃.

42. A compound of formula (I) according to any of numbered embodiments 20 to 41 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:

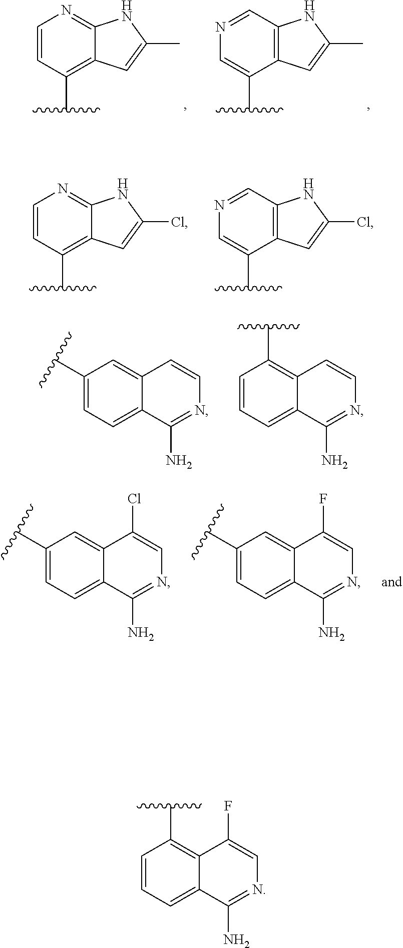

43. A compound of formula (I) according to numbered embodiment 42 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:

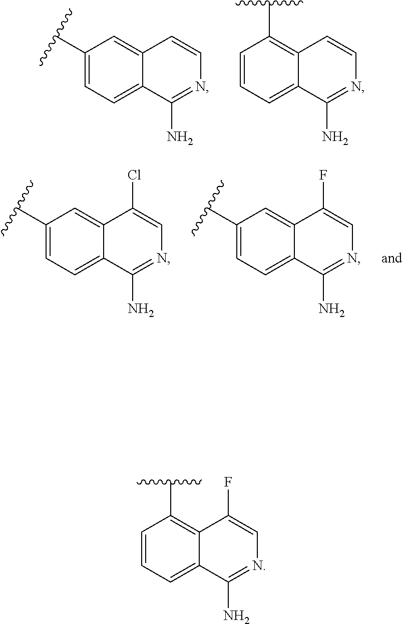

44. A compound of formula (I) according to any of numbered embodiments 20 to 28, 32 to 33, or 40 to 42 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein when B is heteroaryl^{a} and is a 9-membered bicyclic aromatic ring containing a 5-membered ring fused to a 6-membered ring and B is attached to Y via the 6-membered ring, the 9-membered bicyclic aromatic ring contains 1 or 2 ring members independently selected from N, NR12, S and O; and is optionally substituted as for heteroaryl^{a}.

45. A compound of formula (I) according to any of numbered embodiments 20 to 28, 32 to 33, or 40 to 42 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein when B is heteroaryl^{a} and is selected from 6-azaindolyl

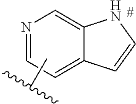

and 7-azaindolyl

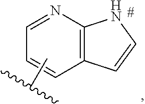

B is optionally substituted as for heteroaryl^{a}, and any optional substituents are, where possible, at any ring member apart from the ring member marked #.

46. A compound of formula (I) according to any of numbered embodiments 1 to 45 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 0 or 1.

47. A compound of formula (I) according to any of numbered embodiments 1 to 45 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 1 or 2.

48. A compound of formula (I) according to numbered embodiment 46 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 0.

49. A compound of formula (I) according to any of numbered embodiments 46 or 47 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 1.

50. A compound of formula (I) according to any of numbered embodiments 1 to 49 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is independently selected from CH₃, OH, CH₂OH, OCH₃, OiPr, CF₃, F, Cl, (CH₂)₀₋₆COOH, CN, CH₂F, CHF₂, CH₂OCH₃ and

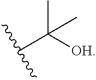 [SMILES: *C(C)(C)O]

51. A compound of formula (I) according to numbered embodiment 50 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is independently selected from F, CH₂OH and OH

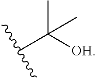 [SMILES: *C(C)(C)O]

52. A compound of formula (I) according to numbered embodiment 51 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is F.

53. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW is selected from -A, —OCH₂-A, —CH₂O-A, —O-A, —(CH₂)₂-A, —NH—CH₂-A and —NH—(CH₂)₂—C(═O)-A.

54. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN;
wherein when A is a bicyclic ring system, the bicyclic ring system is fused, bridged or spiro.

55. A compound of formula (I) according to numbered embodiment 54 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 4- to 12-membered mono- or bi-cyclic ring system, containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

56. A compound of formula (I) according to numbered embodiment 55 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 4- to 12-membered mono- or bi-cyclic ring system, containing one N ring member and optionally one or two further N ring members, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, CN; wherein when A is a bicyclic ring system, the bicyclic ring system is fused, bridged or spiro.

57. A compound of formula (I) according to numbered embodiment 54 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

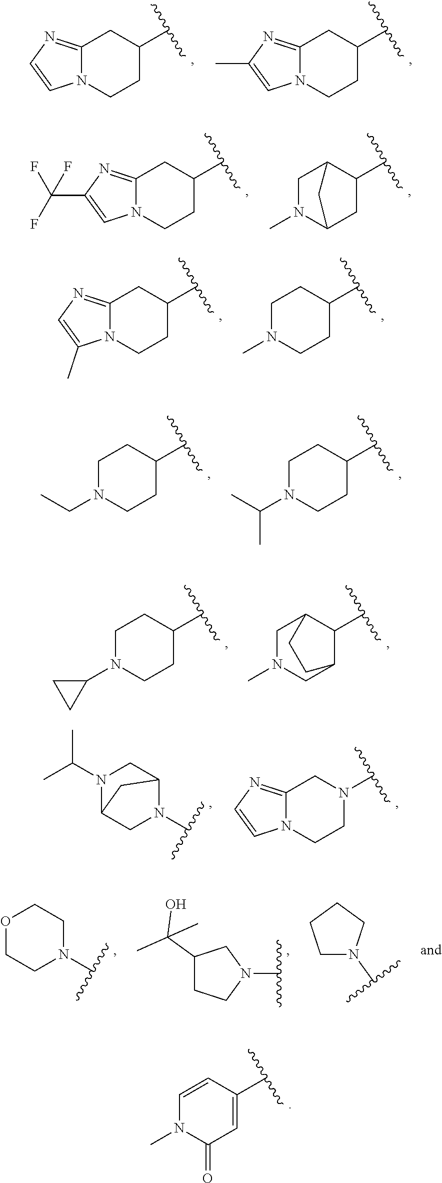

58. A compound of formula (I) according to numbered embodiment 57 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

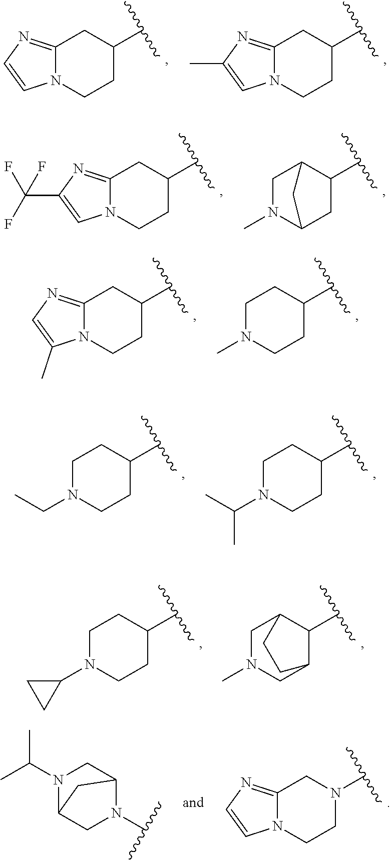

59. A compound of formula (I) according to numbered embodiment 56 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

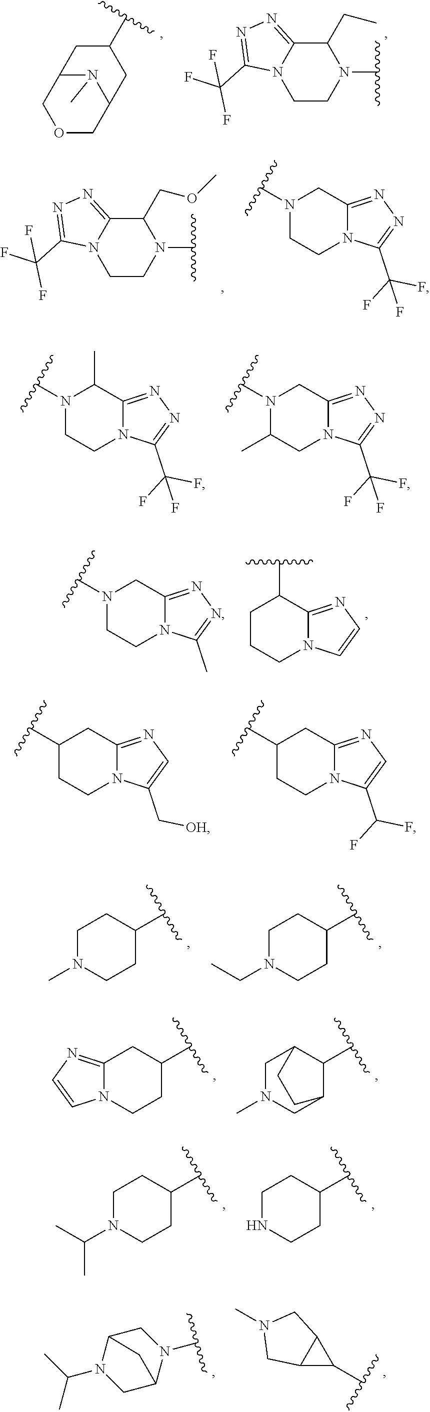

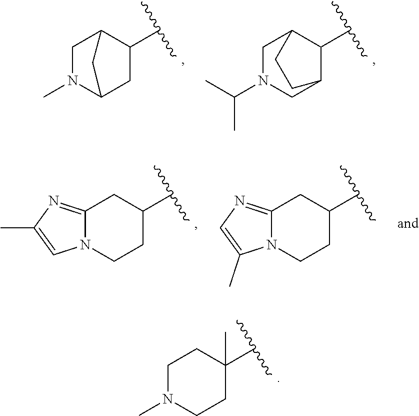

60. A compound of formula (I) according to numbered embodiment 59 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

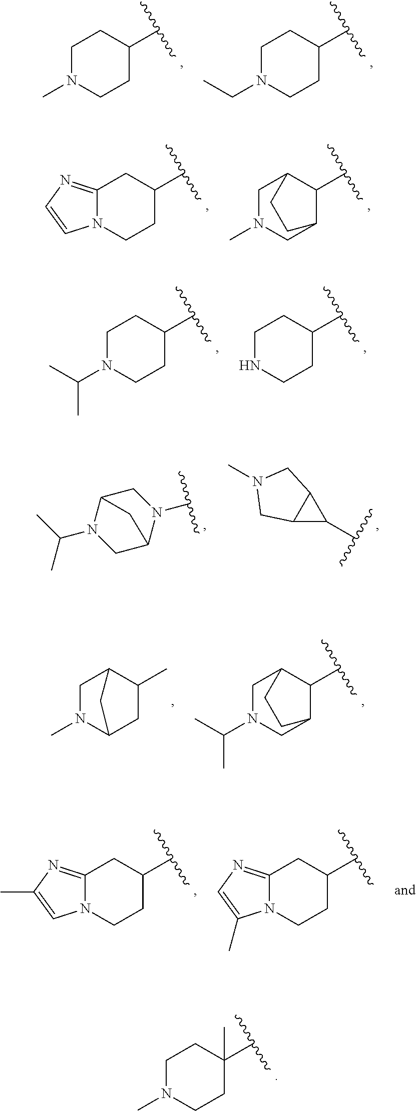

61. A compound of formula (I) according to numbered embodiment 59 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

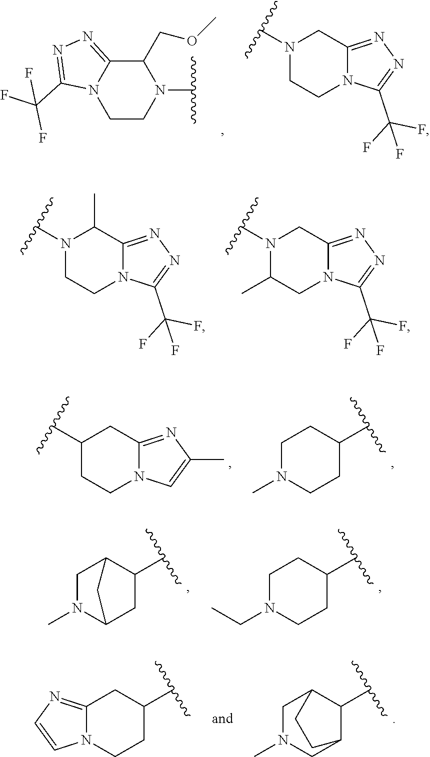

62. A compound of formula (I) according to numbered embodiment 61 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

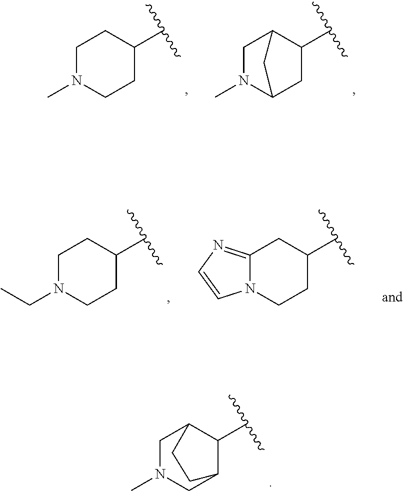

63. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein heteroaryl^{b} is heteroaryl^{c}; and
heteroaryl_{c} is a 5, 6, 9 or 10 membered mono- or bi-cyclic aromatic ring, containing, where possible, 1 or 2 ring members independently selected from N, NR12, S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃.

64. A compound selected from Table 1a, 1b, 2a, 2b, 3, 4a, 4b, 5a, 5b, 6, 7, 8a, 8b, 8c, 9, and 10, or a pharmaceutically acceptable salt, solvate, or solvate of a salt thereof.

65. A compound selected from Table 1a, 2a, 3, 4a, 5a, 6, 7, and 8a, or a pharmaceutically acceptable salt, solvate, or solvate of a salt thereof.

66. A pharmaceutical composition comprising: a compound, or a pharmaceutically acceptable salt and/or solvate thereof, according to any of numbered embodiments 1 to 65, and at least one pharmaceutically acceptable excipient.

67. A compound, or a pharmaceutically acceptable salt and/or solvate thereof, as defined in any of numbered embodiments 1 to 65, or the pharmaceutical composition according to numbered embodiment 66, for use in medicine.

68. The use of a compound, or a pharmaceutically acceptable salt and/or solvate thereof, as defined in any of numbered embodiments 1 to 65, or the pharmaceutical composition according to numbered embodiment 66, in the manufacture of a medicament for the treatment or prevention of a disease or condition in which Factor XIIa activity is implicated.

69. A method of treatment of a disease or condition in which Factor XIIa activity is implicated comprising administration to a subject in need thereof a therapeutically effective amount of a compound, or a pharmaceutically acceptable salt and/or solvate thereof, as defined in any of numbered embodiments 1 to 65, or the pharmaceutical composition according to numbered embodiment 66.

70. A compound, or a pharmaceutically acceptable salt and/or solvate thereof, as defined in any of numbered embodiments 1 to 65, or the pharmaceutical composition according to numbered embodiment 66, for use in a method of treatment of a disease or condition in which Factor XIIa activity is implicated.

71. The use of numbered embodiment 68, the method of numbered embodiment 69, or a compound, a pharmaceutically acceptable salt and/or solvate thereof, or a pharmaceutical composition for use as defined in numbered embodiment 70, wherein the disease or condition in which Factor XIIa activity is implicated is a bradykinin-mediated angioedema.

72. The use of numbered embodiment 71, the method of numbered embodiment 71, or a compound, a pharmaceutically acceptable salt and/or solvate thereof, or a pharmaceutical composition for use as defined in numbered embodiment 71, wherein the bradykinin-mediated angioedema is hereditary angioedema.

73. The use of numbered embodiment 71, the method of numbered embodiment 71, or a compound, a pharmaceutically acceptable salt and/or solvate thereof, or a pharmaceutical composition for use as defined in claim 71, wherein the bradykinin-mediated angioedema is non hereditary.

74. The use of numbered embodiment 68, the method of numbered embodiment 69, or a compound, a pharmaceutically acceptable salt and/or solvate thereof, or a pharmaceutical composition for use as defined in numbered embodiment 70, wherein the disease or condition in which Factor XIIa activity is implicated is selected from vascular hyperpermeability; stroke including ischemic stroke and haemorrhagic accidents; retinal edema; diabetic retinopathy; DME; retinal vein occlusion; and AMD.

75. The use of numbered embodiment 68, the method of numbered embodiment 69, or a compound, a pharmaceutically acceptable salt and/or solvate thereof, or a pharmaceutical composition for use as defined in numbered embodiment 70, wherein, the disease or condition in which Factor XIIa activity is implicated is a thrombotic disorder.

76. The use of numbered embodiment 75, the method of numbered embodiment 75, or a compound, a pharmaceutically acceptable salt and/or solvate thereof, or a pharmaceutical composition for use as defined in numbered embodiment 75, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; atherosclerosis; COVID-19; acute respiratory distress syndrome (ARDS); idiopathic pulmonary fibrosis (IPF); rheumatoid arthritis (RA); and cold-induced urticarial autoinflammatory syndrome.

77. The use of numbered embodiment 68, the method of numbered embodiment 69, or a compound, a pharmaceutically acceptable salt and/or solvate thereof, or a pharmaceutical composition for use as defined in numbered embodiment 70, wherein, the disease or condition in which Factor XIIa activity is implicated is selected from neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

78. The use of any of numbered embodiments 68 or 71 to 77, the method of any of numbered embodiments 69 or 71 to 77, or a compound, a pharmaceutically acceptable salt and/or solvate thereof, or a pharmaceutical composition for use as defined in any of numbered embodiments 70 or 71 to 77, wherein the compound targets FXIIa.

79. A compound of formula (II),

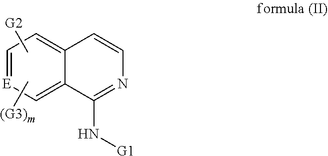

wherein:
   E is selected from CH and N;
   G1 is either:

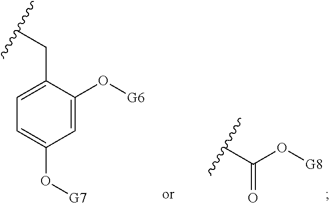

G2 is F, Cl, or Br;
m is 0, 1 or 2;
G3, when present, is independently selected from alkyl, OH, OCF₃, aryl^{b}, heteroaryl^{b}, alkoxy, CF₃, CN, —(CH₂)₀₋₃—N(G4)(G5), —C(═O)OR12, —C(═O)NR13R14 and halo; provided that when m is 1, G3 is not methyl;
G4 and G5 are independently selected from alkyl^{b}, aryl^{b} and heteroaryl^{b} or G4 and G5 together with the nitrogen atom to which they are attached form a carbon-containing 4-, 5-, 6- or 7-membered heterocyclic ring, optionally containing an additional heteroatom selected from N, NR12, S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
G6 and G7 are independently selected from methyl, ethyl, n-propyl and i-propyl;
G8 is selected from methyl, ethyl, n-propyl, i-propyl, n-butyl and i-butyl;
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from (C₁-C₆)alkoxy, OH, —NR13R14, —C(═O)OR13, —C(═O)NR13R14, CN, CF₃, halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from (C₁-C₆)alkoxy, OH, CN, CF₃, halo;
aryl^{b} is phenyl, biphenyl or naphthyl; aryl^{b} may be optionally substituted with 1, 2 or 3 substituents independently selected from alkyl^{b}, alkoxy, OH, halo, CN, and CF₃;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, halo;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, and fluoro;
halo is F, Cl, Br, or I;
heteroaryl is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃;
heteroaryl^{a} is a 5, 6, 9 or 10 membered mono- or bi-cyclic aromatic ring, containing, where possible, 1, 2, 3 or 4 ring members independently selected from N, NR12, S and O;
heteroaryl^{a} may be optionally substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃;
heteroaryl^{b} is a 5, 6, 9 or 10 membered mono- or bi-cyclic aromatic ring, containing, where possible, 1, 2 or 3 ring members independently selected from N, NR12, S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃;
heterocycloalkyl is a non-aromatic carbon-containing monocyclic ring containing 5, 6, or 7 ring members, wherein one or two ring members are independently selected from N, NR8, S, SO, SO₂, and O; wherein heterocycloalkyl may be optionally substituted with 1, 2, or 3 substituents independently selected from alkyl, alkoxy, OH, OCF₃, halo, oxo and CN;
R8 is independently selected from H, alkyl, cycloalkyl, or heterocycloalkyl^{a}; heterocycloalkyl^{a} is a non-aromatic carbon-containing monocyclic ring containing 3, 4, 5, or 6, ring members, wherein at least one ring member is independently selected from N, NR12, S, and O; heterocycloalkyl^{a} may be optionally be substituted with 1 or 2 substituents independently selected from alkyl, (C₁-C₆)alkoxy, OH, CN, CF₃, halo;
R12 is independently selected from H, alkyl, or cycloalkyl;
R13 and R14 are independently selected from H, alkyl^{b}, aryl^{b} and heteroaryl^{b} or R13 and R14 together with the nitrogen atom to which they are attached form a carbon-containing 4-, 5-, 6- or 7-membered heterocyclic ring, optionally containing an additional heteroatom selected from N, NR12, S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
and tautomers, isomers, stereoisomers (including enantiomers, diastereoisomers and racemic and scalemic mixtures thereof), deuterated isotopes, and salts and/or solvates thereof.

80. A compound of formula (II) according to numbered embodiment 79 or a tautomer, isomer, stereoisomer (including an enantiomer, a diastereoisomer and a racemic and scalemic mixture thereof), a deuterated isotope, and a salt and/or solvate thereof,
wherein when m is 0; G2 is substituted at any ring member apart from the ring member marked **

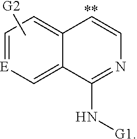

81. A compound of formula (II) according to any of numbered embodiments 79 or 80 or a tautomer, isomer, stereoisomer (including an enantiomer, a diastereoisomer and a racemic and scalemic mixture thereof), a deuterated isotope, and a salt and/or solvate thereof,
wherein G8 is selected from methyl, n-propyl, i-propyl, n-butyl and i-butyl.

82. A compound of formula (II) according to any of numbered embodiments 79-81 or a tautomer, isomer, stereoisomer (including an enantiomer, a diastereoisomer and a racemic and scalemic mixture thereof), a deuterated isotope, and a salt and/or solvate thereof,
wherein G2 is selected from Cl and Br.

83. A compound of formula (II) according to numbered embodiment 82 or a tautomer, isomer, stereoisomer (including an enantiomer, a diastereoisomer and a racemic and scalemic mixture thereof), a deuterated isotope, and a salt and/or solvate thereof,
wherein G2 is Cl.

84. A compound of formula (II) according to numbered embodiment 82 or a tautomer, isomer, stereoisomer (including an enantiomer, a diastereoisomer and a racemic and scalemic mixture thereof), a deuterated isotope, and a salt and/or solvate thereof,
wherein G2 is Br.

85. A compound of formula (II) according to any of numbered embodiments 79 to 84 or a tautomer, isomer, stereoisomer (including an enantiomer, a diastereoisomer and a racemic and scalemic mixture thereof), a deuterated isotope, and a salt and/or solvate thereof,
wherein m is 0 or 1.

86. A compound of formula (II) according to any of numbered embodiments 79 to 85 or a tautomer, isomer, stereoisomer (including an enantiomer, a diastereoisomer and a racemic and scalemic mixture thereof), a deuterated isotope, and a salt and/or solvate thereof,
wherein m is 1.

87. A compound of formula (II) according to any of numbered embodiments 79 to 86 or a tautomer, isomer, stereoisomer (including an enantiomer, a diastereoisomer and a racemic and scalemic mixture thereof), a deuterated isotope, and a salt and/or solvate thereof,
wherein G3 is selected from alkyl, alkoxy, OH, OCF₃, halo, CN, and CF₃.

88. A compound of formula (II) according to numbered embodiment 87 or a tautomer, isomer, stereoisomer (including an enantiomer, a diastereoisomer and a racemic and scalemic mixture thereof), a deuterated isotope, and a salt and/or solvate thereof,
wherein G3 is halo.

89. A compound of formula (II) according to numbered embodiment 88 or a tautomer, isomer, stereoisomer (including an enantiomer, a diastereoisomer and a racemic and scalemic mixture thereof), a deuterated isotope, and a salt and/or solvate thereof,
wherein G3 is selected from Cl and F.

90. A compound of formula (II) according to numbered embodiment 89 or a tautomer, isomer, stereoisomer (including an enantiomer, a diastereoisomer and a racemic and scalemic mixture thereof), a deuterated isotope, and a salt and/or solvate thereof,
wherein G3 is Cl.

91. A compound of formula (II) according to numbered embodiment 89 or a tautomer, isomer, stereoisomer (including an enantiomer, a diastereoisomer and a racemic and scalemic mixture thereof), a deuterated isotope, and a salt and/or solvate thereof,
wherein G3 is F.

92. A compound of formula (II) according to any of numbered embodiments 79 to 85 or a tautomer, isomer, stereoisomer (including an enantiomer, a diastereoisomer and a racemic and scalemic mixture thereof), a deuterated isotope, and a salt and/or solvate thereof,
wherein m is 0.

93. A compound of formula (II) according to any of numbered embodiments 79 to 92 or a tautomer, isomer, stereoisomer (including an enantiomer, a diastereoisomer and a racemic and scalemic mixture thereof), a deuterated isotope, and a salt and/or solvate thereof,
wherein E is CH.

94. A compound of formula (II) according to any of numbered embodiments 79 to 92 or a tautomer, isomer, stereoisomer (including an enantiomer, a diastereoisomer and a racemic and scalemic mixture thereof), a deuterated isotope, and a salt and/or solvate thereof,
wherein E is N.

95. A compound of formula (II) according to any of numbered embodiments 79 to 94 or a tautomer, isomer, stereoisomer (including an enantiomer, a diastereoisomer and a racemic and scalemic mixture thereof), a deuterated isotope, and a salt and/or solvate thereof,
wherein G1 is selected from 3

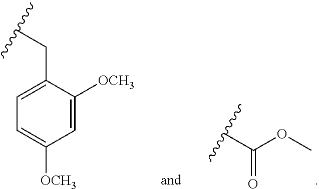

96. A compound of formula (II) according to numbered embodiment 95 or a tautomer, isomer, stereoisomer (including an enantiomer, a diastereoisomer and a racemic and scalemic mixture thereof), a deuterated isotope, and a salt and/or solvate thereof, OCH₃
wherein G1 is

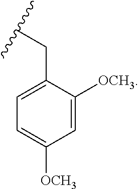 [SMILES: *Cc1ccc(OC)cc1OC]

97. A compound of formula (II) according to numbered embodiments 95 or a tautomer, isomer, stereoisomer (including an enantiomer, a diastereoisomer and a racemic and scalemic mixture thereof), a deuterated isotope, and a salt and/or solvate thereof,
wherein G1 is

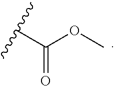 [SMILES: *C(=O)OC]

98. A compound selected from

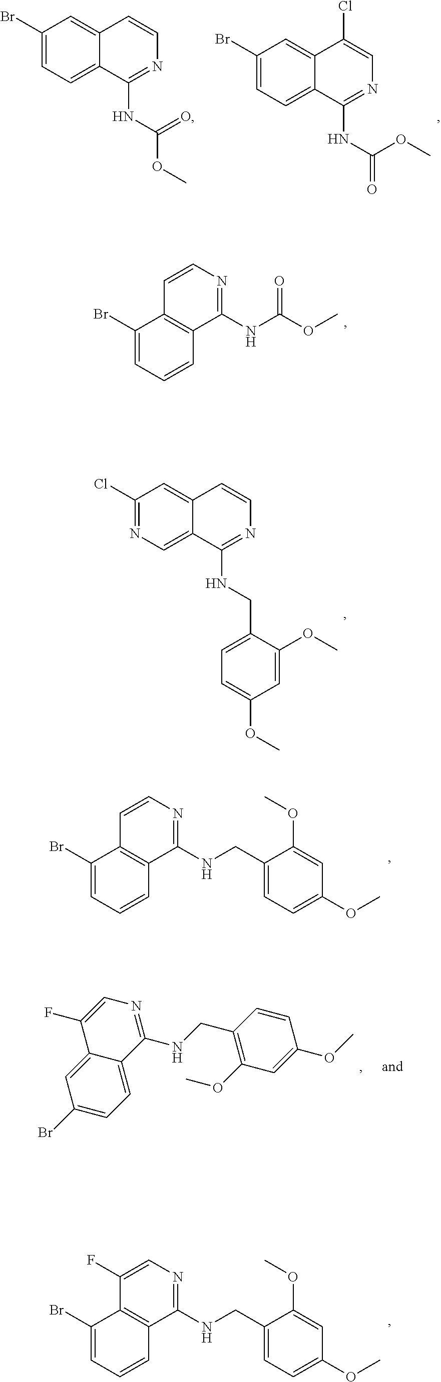

or a salt, solvate, or solvate of a salt thereof.

Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. Those skilled in the art will readily understand that known variations of the conditions, processes and order in which the synthetic steps are performed in the following preparative procedures can be used to prepare these compounds.

The compounds and intermediates of the invention may be isolated in the form of their pharmaceutically acceptable salts, such as those described previously herein above. The interconversion between free form and salt form would be readily known to those skilled in the art.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (boc), which is readily removed by treatment with an acid such as trifluoroacetic acid or hydrogen chloride in an organic solvent such as dichloromethane. Alternatively the amino protecting group may be a benzyloxycarbonyl (Cbz or Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether, deprotection conditions comprise refluxing in 48% aqueous HBr, or by stirring with borane tribromide in an organic solvent such as DCM. Alternatively where a hydroxy group is protected as a benzyl ether, deprotection conditions comprise hydrogenation with a palladium catalyst under a hydrogen atmosphere.

The graphic representations of racemic, ambiscalemic and scalemic or enantiomerically pure compounds used herein are taken from Maehr J. Chem. Ed. 62, 114-120 (1985): solid wedges ( [symbol] ) and broken wedges ( [symbol] ) are used to denote the absolute configuration of a chiral element; wavy lines ( [symbol] ) indicate disavowal of any stereochemical implication which the bond it represents could generate; solid bold lines ( [symbol] ) and broken bold lines ( [symbol] ) are geometric descriptors indicating the relative configuration shown, but denoting racemic character; and wedge outlines ( [symbol] ) and broken lines ( [symbol] ) denote enantiomerically pure compounds of indeterminate absolute configuration. For nomenclature in the text corresponding to wedge outlines ( [symbol] ) and broken lines ( [symbol] ), we define R* and S* as indicating single enantiomers of uncertain absolute configuration.

Thus, for example, in examples 4267 and 4412 below, the synthesis of 6-N-({2-[(7S*)-5H,6H,7H,8H-imidazo[1,2-a]pyridin-7-ylmethoxy]pyridin-4-yl}methyl)isoquinoline-1,6-diamine and 6-N-({2-[(7R*)-5H,6H,7H,8H-imidazo[1,2-a]pyridin-7-ylmethoxy]pyridin-4-yl}methyl)isoquinoline-1,6-diamine are described. The (R*) and (S*) are intended to indicate that the product is a single enantiomer possessing the characteristics described (eq. NMR, HPLC, retention time etc), in which each of the chiral centres is believed on the basis of circumstantial evidence to be of the configuration shown, but the absolute configuration has not been confirmed. Thus, for example compound 4267, the depiction:

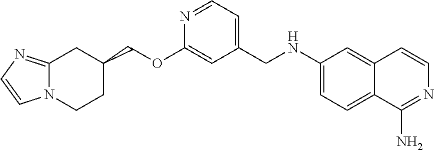 [SMILES: Nc1nccc2cc(NCc3ccnc(OC[C@H]4CCn5ccnc5C4)c3)ccc12]

means that the compound is a single one of the following two stereoisomers, and probably the first:

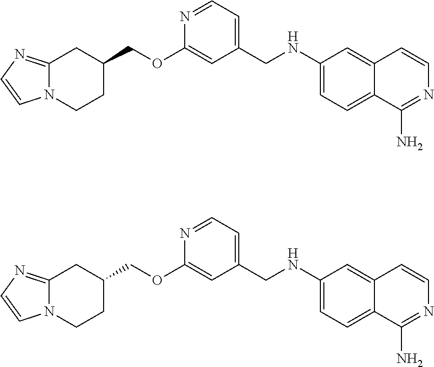 [SMILES: Nc1nccc2cc(NCc3ccnc(OC[C@H]4CCn5ccnc5C4)c3)ccc12 ; Nc1nccc2cc(NCc3ccnc(OC[C@@H]4CCn5ccnc5C4)c3)ccc12]

As used herein, a depiction including wedges or broken lines (eg.

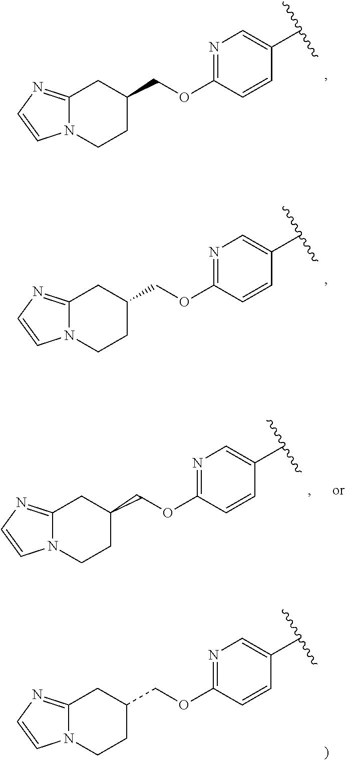 [SMILES: *c1ccc(OC[C@H]2CCn3ccnc3C2)nc1 ; *c1ccc(OC[C@@H]2CCn3ccnc3C2)nc1 ; *c1ccc(OC[C@H]2CCn3ccnc3C2)nc1 ; *c1ccc(OC[C@@H]2CCn3ccnc3C2)nc1]

indicates that the structure encompasses purity of that relative or absolute configuration of at least 80% ee, preferably >90% ee.

As used herein, when a compound possesses a centre of asymmetry, its depiction with simple lines (eg.

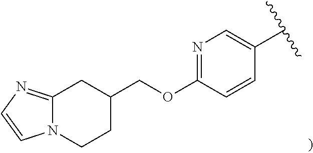 [SMILES: *c1ccc(OCC2CCn3ccnc3C2)nc1]

indicates that the structure includes any and all stereoisomers, without regard to enantiomeric purity.

The invention is illustrated by the following non-limiting examples in which the following abbreviations and definitions are used:

| | |
|---|---|
| AcOH | acetic acid |
| aq | aqueous solution |
| AIBN | azobisisobutyronitrile |
| boc | tert-butoxy carbonyl |
| Boc₂O | di-tert-butyl dicarbonate |
| BrettPhos Pd G3 | [(2-di-cyclohexylphosphino-3,6-dimethoxy-2′,4′,6′- triisopropyl-1,1′- |
| | biphenyl)-2-(2′-amino-1,1′-biphenyl)]palladium(II) methanesulfonate |
| BrettPhos Pd G4 | dicyclohexyl-[3,6-dimethoxy-2-[2,4,6-tri(propan-2- |
| | yl)phenyl]phenyl]phosphane; methanesulfonic acid; N-methyl-2- |
| | phenylaniline; palladium |
| tBu | tert-butyl |
| tBuBrettPhos Pd G3 | [(2-di-tert-butylphosphino-3,6-dimethoxy-2′,4′,6′-triisopropyl-1,1′- |
| | biphenyl)-2-(2′-amino-1,1′-biphenyl)]palladium(II) methanesulfonate |
| Cbz | benzyl carbamate |
| CDI | 1,1′-carbonyldiimidazole |
| Celite ® | Filter agent (diatomaceous earth) |
| DCM | dichloromethane |
| DIAD | diisopropyl azodicarboxylate |
| DIPEA | N,N-diisopropylethylamine |
| DMF | N,N-dimethylformamide |
| DMSO | dimethyl sulfoxide |
| EDC | 1-ethyl-3-(3-dimethylaminopropyl)carbodiimide hydrochloride |
| eq | equivalent |
| Et₂O | diethyl ether |
| Et | ethyl |
| EtOH | ethanol |
| EtOAc | ethyl acetate |
| HATU | 2-(3H-[1,2,3]triazolo[4,5-b]pyridin-3-yl)-1,1,3,3-tetramethylisouronium |
| | hexafluorophosphate(V) |
| h | Hours |
| HOBt | 1-hydroxybenzotriazole hydrate |
| LCMS | Liquid chromatography mass spectrometry |
| Me | methyl |
| MeCN | acetonitrile |
| MeOH | methanol |
| min | minutes |
| MS | mass spectrum |
| Ms | methanesulfonyl |
| MsCl | methanesulfonyl chloride |
| NBS | N-bromosuccinimide |
| NCS | N-chlorosuccinimide |
| NMR | nuclear magnetic resonance spectrum |
| NMP | N-methyl-2-pyrrolidone |
| OAc | acetate |
| Pd₂(dba)₃ | tris(dibenzylideneacetone)dipalladium(0) |
| Pet. Ether | petroleum ether fraction boiling at 60-80° C. |
| Ph | phenyl |
| iPr | iso-propyl |
| nPr | n-propyl |
| RuPhos | 2-dicyclohexylphosphino-2′,6′-diisopropoxybiphenyl |
| RuPhos Pd G3 | (2-dicyclohexylphosphino-2′,6′-diisopropoxy-1,1′-biphenyl)[2-(2′-amino- |
| | 1,1′-biphenyl)]palladium(II) methanesulfonate |
| sat. | saturated |
| SCX | strong cation exchange cartridge |
| SWFI | sterile water for injection |
| rt | room temperature |
| TBAB | tetra-n-butylammonium bromide |
| TBAF | tetra-n-butylammonium fluoride |
| TBDMS | tert-butyldimethylsilyl |
| TBME | tert-butyl methyl ether |
| THF | tetrahydrofuran |
| TEA | triethylamine |
| TFA | trifluoroacetic acid |
| Z | benzyl carbamate |

All reactions were carried out under an atmosphere of nitrogen unless specified otherwise.

Hydrogenations were typically carried out using an H-Cube® reactor (manufactured by Thalesnano, Inc, Hungary).

References to the use of microwave, a microwave reactor, microwave heating and microwave irradiation all refer to the use of a CEM Discover Microwave Reactor.

References to the use of a phase separator refer to columns fitted with a selectively permeable, optimized frit material that separates aqueous phase from an organic phase under gravity.

¹H NMR spectra were recorded on a Bruker (500 MHz or 400M Hz) spectrometer and reported as chemical shift (ppm).

Molecular ions were obtained using LCMS with appropriate conditions selected from
Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min;
Agilent, X-Select, acidic, 5-95% MeCN/water over 4 min. Data was collected using a Thermofinnigan Surveyor MSQ mass spectrometer with electrospray ionisation in conjunction with a Thermofinnigan Surveyor LC system;
LCMS (Waters Acquity UPLC, C18, Waters X-Bridge UPLC C18, 1.7 μm, 2.1×30 mm, Basic (0.1% Ammonium Bicarbonate) 3 min method;
LCMS (Agilent, X-Select, Waters X-Select C18, 2.5 μm, 4.6×30 mm, Acidic 4 min method, 95-5 MeCN/water);
LCMS (Agilent, Basic, Waters X-Bridge C18, 2.5 μm, 4.6×30 mm, Basic 4 min method, 5-95 MeCN/water;
Acquity UPLC BEH C18 1.7 μM column, 50×2.1 mm, with a linear gradient 10% to 90% 0.1% HCO2H/MeCN into 0.1% HCO2H/H2O over 3 min, flow rate 1 mL/min. Data was collected using a Waters Acquity UPLC mass spectrometer with quadropole dalton, photodiode array and electrospray ionisation detectors.

Flash chromatography was typically carried out over ‘silica’ (silica gel for chromatography, 0.035 to 0.070 mm (220 to 440 mesh) (e.g. Merck silica gel 60)), and an applied pressure of nitrogen up to 10 p.s.i accelerated column elution. Alternatively, pre-prepared cartridges of silica gel were used.

The term “prep HPLC” refers to reverse phase preparative HPLC purifications.

The procedure of lyophilisation (or freeze drying) is generally well known in the art. Typically the substance is taken up in water, if necessary with the addition of a minimum amount of MeCN to aid dissolution, and frozen, typically by rapid cooling in a cold bath at −78° C. The resulting frozen solid mixture is evaporated to dryness in vacuo.

The term “concentrated” refers to evaporation of solvent under reduced pressure using a rotary evaporator, heating where necessary.

All solvents and commercial reagents were used as received.

IUPAC chemical names were generated using automated software such as Lexichem's automatic chemical naming from OpenEye Scientific Software, Inc, provided as a component of Dotmatics Studies Notebook.

Other automated software used for naming include ChemDraw (PerkinElmer) or the Chemaxon software provided as a component of MarvinSketch or as a component of the IDBS E-WorkBook. The example compounds described herein can be prepared using conventional synthetic methods for example, but not limited to, the routes outlined in the General Schemes below, using, for example, the General Methods below.

General Methods

1. General Method 1 (GM1): S_{N}Ar Alkylation (O and N)

a. General Method 1a (GM1a): S_{N}Ar O-Alkylation Using NaH

To a suspension of NaH (60% wt. on mineral oil) (1.04 eq) in DMF in an ice/water bath was added a solution of alcohol (1.02 eq) in DMF dropwise over 2 min. The mixture was allowed to warm to rt for 5 min before cooling again in an ice/water bath and treating with pyridyl halide (1.0 eq). The reaction mixture was maintained in an ice/water bath for 1 h then warmed to rt for 18 h. The reaction mixture was cooled in an ice/water bath and sat. Na₂CO₃ (aq) was added followed by water. This was extracted with EtOAc (×3) and the organic phases were combined, washed with 1:1 water/brine and brine. The organic phase was dried (MgSO₄), filtered and concentrated. The crude product was purified by flash chromatography.

b. General Method 1b (GM1b): S_{N}Ar O-Alkylation Using Cs₂CO₃

To a solution of alcohol (1.0 eq) and pyridyl halide (1.0 eq) in MeCN was added Cs₂CO₃ (2.0 eq) and the mixture was stirred in a sealed vial at 50° C. for 18-72 h. The product was isolated and purified using one of the following methods
i) The reaction mixture was cooled to rt and diluted with water (10 mL). The crude product was extracted into DCM, dried (MgSO₄), filtered and concentrated. The residue was purified by flash chromatography
ii) The reaction mixture was filtered through Celite® and the filtrate was concentrated to yield the crude product which was either used without further purification or purified by flash chromatography
iii) The crude reaction mixture was passed directly through an SCX in MeOH. The SCX was washed with MeOH and the product was eluted with 7M NH₃ in MeOH. The crude product was purified by flash chromatography.

c. General Method 1c (GM1c): S_{N}Ar O-Alkylation Using NaOtBu

A solution of alcohol (1.0 eq), aryl bromide (1.0 eq) and NaOtBu (3.0 eq) in NMP was stirred in the microwave at 140° C. for 4 h. The crude reaction mixture was loaded onto an SCX in MeOH and washed with MeOH and the product was eluted with 7M NH₃ in MeOH (50 mL). The product was concentrated and purified by flash chromatography or prep HPLC.

d. General Method 1d (GM1d): S_{N}Ar N-Alkylation

Amine (1.0 eq) (106 mg, 0.82 mmol) and halopyridine (1.0 eq) (100 mg, 0.82 mmol) were dissolved in MeCN (3 mL). K₂CO₃ (3.0 eq) (340 mg, 2.46 mmol) was added and the reaction was stirred at 60-120° C. for 60-90 min under thermal heating or microwave irradiation. The reaction was diluted with water and extracted with iso-propanol/CHCl₃ (1:10) (×3). The combined organics were washed with brine, dried (MgSO₄) and concentrated. The product was isolated and used directly or purified by flash chromatography.

2. General Method 2 (GM2): Cyanation

The aryl bromide (1.0 eq) and Zn(CN)₂ (1.5 eq) and were suspended in NMP. The mixture was degassed with nitrogen for 10 min before Pd(PPh₃)₄(0.15 eq) was added and the mixture was further degassed via 3 vacuum nitrogen cycles. The reaction was heated to 80° C. under N₂ for 16-90 h. The reaction was diluted with EtOAc. The organic phase was washed with sat. NaHCO₃ (aq) (×2) and brine (×3), dried (Na₂SO₄), filtered and concentrated. The product was purified by flash chromatography.

3. General Method 3 (GM3): Reduction

a. General Method 3a (GM3a): nitrile reduction; H-Cube® with Pd/C or Raney Ni cartridge

The nitrile was dissolved in a 0.5M NH₃/MeOH solution passed through an H-Cube® reactor (Pd/C or Raney Ni cartridge), typical conditions: 50° C., ‘full’ hydrogen delivery mode (50 bar), flow rate: 1 mL/min. The reaction was concentrated to afford the product which was used without further purification.

b. General Method 3b (GM3b): Nitrile, Amide and Ester Reduction; LiAlH₄ in THF To a solution of amide, nitrile, or ester (1.0 eq) in THF in an ice/water bath was added LiAlH₄ (2M in THF) (2.0 eq) dropwise and the reaction mixture was allowed to warm to rt then stirred for 4-18 h. The reaction mixture was cooled in an ice/water bath, treated portionwise with Na₂SO₄·10H₂O (3.5 eq) and stirred for 30 min before being dried (MgSO₄),filtering and washing with THF (10 mL). The filtrate was concentrated to afford the crude product which was used without purification or purified by flash chromatography.

c. General Method 3c: Borane-THF

A solution of nitrile (1.0 eq) in THF was cooled in an ice/water bath before borane (1M in THF, 2.0 eq) was added dropwise. The reaction was allowed to warm to rt then heated to 60° C. for 16-96 h. MeOH was added and heating continued at 60° C. for 24 h before cooling to rt and concentrating. The product was isolated and purified using one of the following methods:
i) The crude product was loaded onto an SCX in MeOH and washed with MeOH. The product was eluted with 7M NH₃ in MeOH and the eluent concentrated.
ii) The crude product was purified by flash chromatography
iii) Boc₂O (1.2 eq) was added to the crude reaction mixture and stirred overnight. The solvent was evaporated in vacuo. The product was taken up in DCM, washed with water and brine, dried (Na₂SO₄), filtered and concentrated. The boc-protected amine was either used without further purification or purified by flash chromatography

d. General Method 3d: NiCl₂

A solution of nitrile (1.0 eq), NiCl₂·6H₂O (1.0 eq) and Boc₂O (3.0 eq) in MeOH was cooled in an ice/water bath and NaBH₄ (5.0 eq) added portionwise. The reaction was allowed to warm to rt and stirred for 18 h. Water was added and the reaction mixture filtered, washed with THF and concentrated. The crude product was purified by flash chromatography.

e. General Method 3e: Hydrogenation; Pd/C

To a solution of nitrile (1.0 eq) in MeOH or EtOH under an inert atmosphere was added 10% Pd/C (0.1-0.2 eq). Additives such as HCl, sulfuric acid, or Boc₂O may optionally be added. The reaction was stirred under an atmosphere of H₂ (g) for 2-72 h. The catalyst was removed by filtration over Celite®, which was washed with EtOH. The product was isolated following concentration of the filtrate and used directly or purified by flash chromatography.

f. General Method 3f: Ring Saturation Reduction

A biaryl ring (1.0 eq) was dissolved in EtOH and subjected to hydrogenation in the H-Cube® at 70° C., 50 bar, 1 mL/min using a 10% Pd/C CatCart, recirculating when necessary. The solvent was removed in vacuo to afford the product which was used without purification.

4. General Method 4 (GM4): Buchwald

A suspension of benzylamine or heteroarylamine (1.0 eq), aryl halide (1.1 eq) and a base such as Cs₂CO₃ or NaOtBu (2.0 eq) in a degassed solvent such as THF or 1,4-dioxane was purged with N₂ (g). BrettPhos Pd G3 (0.11 eq) was added (or otherwise Ruphos Pd G3 where indicated) and the mixture degassed and purged with N₂ (g) for 5 min. The reaction was heated in a sealed vial at rt −80° C. for 30 min-3 days as required. The product was isolated and purified using one of the following methods:
i) The reaction was quenched with AcOH (2.0 eq) and concentrated. The crude was purified by an SCX eluting with NH₃ in MeOH followed by purification by flash chromatography or prep HPLC.
ii) The reaction was quenched with AcOH (2.0 eq), filtered through Celite®, washing with EtOAc and the filtrate concentrated. The crude product was purified by flash chromatography
iii) The reaction mixture was acidified with AcOH (2.0 eq) and stirred for 5 min, 1M NH₃ in MeOH was added and the reaction mixture was concentrated on to silica and purified by flash chromatography.
iv) The reaction mixture was dry loaded on to silica and purified by flash chromatography.

5. General Method 5 (GM5): S_{N}2 Alkylation (0 and N)

a. General Method 5a: S_{N}2 Alkylation: NaH

To a suspension of NaH (60% wt. on mineral oil) (1.1 eq) in DMF in an ice/water bath was added a solution of alcohol (1.0 eq) in DMF dropwise over 2 min. The mixture was allowed to warm to rt for 5 min before cooling again in an ice/water bath and treating with a solution of the alkylhalide (1.0 eq) in DMF over 2 min. The mixture was maintained in an ice/water bath for 1 h before being allowed to warm to rt and stirred for 2-18 h. Sat. NH₄Cl (aq) (50 mL) or sat. NaHCO₃ (aq) was added and extracted with EtOAc (×3). The organic phases were combined, dried (MgSO₄), filtered and concentrated. The crude product was purified by flash chromatography.

b. General Method 5b: S_{N}2 alkylation; Cs₂CO₃ or K₂CO₃

A solution of alkylhalide (1-2 eq) (1.20 g, 4.30 mmol), pyrazole (1.0 eq) and base such as K₂CO₃, or Cs₂CO₃ (2.5 eq) in a solvent such as NMP was stirred in the microwave at 130° C. for 2 h. The reaction was quenched with MeOH (5 mL) and diluted with water (50 mL). The product was extracted into TBME (2×50 mL) and washed with brine (50 mL). The organic layer was dried (Na₂SO₄), filtered and concentrated. The product was either used directly or purified by flash chromatography

6. General Method 6: (GM6): Chlorination

a. General Method 6a (GM6a): Chlorination Via a Mesylate Methane sulfonyl chloride (2.5 eq) (0.6 mL, 8.32 mmol) was added to a solution of TEA (2.8 eq) and alcohol (1.0 eq) in DCM (20 mL) while cooling in an ice/water bath. The reaction was stirred at rt for 18 h. The reaction was diluted with DCM and washed with sat. NaHCO₃ (aq). The aqueous layer was extracted with DCM (3×25 mL) and the combined organics were washed with brine, dried (Na₂SO₄), filtered and concentrated. The crude product was purified by flash chromatography.

b. General Method 6b (GM6b): Chlorination Via NCS

A solution of indole or azaindole (1.0 eq) in dichloroethane was protected from light and treated with NCS (3.75 eq) at rt for 12-48 h. The mixture was treated with 1M HCl (aq) and the phases separated. The organic phase was washed with brine, dried (Na₂SO₄), filtered, concentrated and purified by flash chromatography.

7. General Method 7 (GM7): Boc Deprotection; HCl or TFA

a. General Method 7a: Boc Deprotection; HCl/Dioxane

A suspension of boc protected amine (1.0 eq) in 1,4-dioxane was treated with 4M HCl in dioxane (10.0 eq) was added and the reaction stirred at rt for 2-24 h. The product was isolated and purified using one of the following methods:
I. The reaction mixture was concentrated, optionally azeotroping with Et₂O or toluene to afford the product as a hydrochloride salt.
II. The reaction mixture was concentrated and the product was converted to free base using a bicarbonate cartridge, loading in MeOH. The filtrate was concentrated and triturated with Et₂O to afford the product.

b. General Method 7b: Boc Deprotection; TFA

A mixture of boc protected amine (1.0 eq) in DCM was treated with TFA (10.0 eq) and stirred at rt for 2 h. The mixture was passed directly through an SCX and washed with MeOH. The product was eluted with a solution of 7M NH₃ in MeOH and concentrated. The crude product was purified by flash chromatography or prep HPLC.

8. General Method 8 (GM8): Amide Coupling

To a solution of carboxylic acid (1.03 mmol) in DCM (10 mL) in an ice/water bath was added HOBt (1.1 eq), EDC (1.3 eq) and TEA (5.0 eq). After 10 min, amine (1.0 eq) was added and the mixture stirred at rt for 15 h. The reaction was diluted with DCM and washed with sat. NaHCO₃ (aq) (10 mL), water and brine. The organic layer was dried (MgSO₄), filtered and concentrated. The residue was purified by flash chromatography.

9. General Method 9 (GM9): Reductive Alkylation

To a suspension of amine (1.0 eq) in a solvent such as THF, DCM or DMF was added the aldehyde or ketone (5.0 eq.) and AcOH (2 eq). The reaction was stirred for 15 min before the addition of sodium triacetoxyborohydride (3.0 eq). The mixture was stirred at rt for 20 h then partitioned between EtOAc or DCM and sat. NaHCO₃ (aq). The organic layer was dried (MgSO₄), filtered and concentrated. The residue was purified by flash chromatography.

10. General Method 10 (GM10): Tandem Boc Deprotection and Eschweiler-Clarke Methylation

A solution of boc-protected amine (1.0 eq) in formic acid (10.0 eq) was stirred at 50° C. for 30 min before formaldehyde (37% in water) (2.5 eq) was added and the reaction mixture heated to 90° C. for 1-3 h. The reaction mixture was concentrated. The crude product was dissolved in MeOH and passed directly through an SCX and washed with MeOH (20 mL). The product was eluted with a solution of 7M NH₃ in MeOH (50 mL) and concentrated. The crude product was either used without further purification or purified by flash chromatography.

11. General Method 11 (GM11): Pyridone Chlorination

Pyridone (1.0 eq) was suspended in phosphorus oxychloride (large excess) and heated at reflux for 4 h. The reaction mixture was evaporated then azeotroped with toluene (×2). The residue was used immediately in the next step, taking care to exclude moisture.

12. General Method 12 (GM12): 2,4-Dimethoxybenzyl Deprotection

A solution of 2,4-dimethyoxybenzyl protected amine (1.0 eq) in TFA (10 eq.) was stirred at rt-50° C. for 1 h. The reaction mixture was concentrated. The resulting residue was suspended in MeOH (2 mL) and loaded on to an SCX, which was flushed with MeOH (4×5 mL). The product was eluted with a solution of 1N NH₃ in MeOH (4×5 mL). The solvent was removed in vacuo. The crude product was either used without further purification or purified by flash chromatography or prep HPLC.

13. General Method 13 (GM13): Carbamate Protection

To a solution of aminopyridine (1.0 eq) and TEA (2.0 eq) in DCM (12 mL) in an ice/water bath was added methylchloroformate (3.0 eq) and the reaction was stirred at rt for 48 h. The reaction mixture was diluted with DCM and washed with water (20 mL). The aqueous was extracted with DCM (3×80 mL) and the combined organics dried (Na₂SO₄), filtered and concentrated. The crude product was triturated with EtOAc.

14. General Method 14 (GM14): Carbamate Deprotection

a. General Method 14a: KOH

A mixture of methyl carbamate (1 eq) and KOH (6 eq) in MeOH was stirred at 60° C. for 12-48 h. The product was isolated and purified using one of the following methods:
i) The reaction was quenched with AcOH (6.0 eq) and the mixture was left to stir for 5 min before being concentrated. The residue was passed directly through an SCX in MeOH. The SCX was washed with MeOH and the product was eluted with 7M NH₃ in MeOH and lyophilised.
ii) The reaction was quenched with AcOH (6.0 eq) and the mixture was left to stir for 5 min before being concentrated. The residue was passed directly through an SCX in MeOH. The SCX was washed with MeOH and the product was eluted with 7M NH₃ in MeOH. The product was purified by flash chromatography or prep HPLC
iii) The reaction was quenched with AcOH (6.0 eq), concentrated, and purified by prep HPLC.

b. General Method 14b: LiOH

To a solution of methyl carbamate (1 eq) in THF/water (10:1) was added lithium hydroxide monohydrate (3-5 eq) and the reaction stirred at 60° C. for 18 h-4 days. The mixture was cooled to rt and concentrated. The crude residue was purified via flash chromatography or prep HPLC

15. General Method 15: SEM Deprotection

A mixture of methanesulfonic acid (39.0 eq)) and water (0.1 mL) was added dropwise to a rapidly stirred solution of indole or azaindole (1.0 eq) in DCM. The mixture was stirred at rt for 3 h. The reaction mixture was diluted with DCM (10 mL) and cooled in an ice/water bath before being quenched with dropwise addition of ethylene diamine (10.0 eq) and the mixture was stirred for 2 h. The reaction mixture was concentrated and the crude product was purified by flash chromatography.

General Schemes

Where the central ring is a 6-membered aryl or heteroaromatic ring (for example phenyl, pyridine and pyrazine as shown e.g. by the ring including U and V in General Scheme 1), the same routes and methods described in the general schemes below can be applied regardless of whether the non-R substituents on the central ring (if an R substituent is present) are para or meta to one another. For example, in General Scheme 3, the non-R substituents are those defined as RgA-O- and —CH₂NH—RgD, and in General Scheme 1, there is no “R substituent” so the “non-R substituents” are the groups defined by e.g. Rg-A-Q- and —CH₂NH—RgD.

General Scheme 1 outlines a synthetic route for certain example compounds e.g. those with a 6-membered central ring as defined below, and RgA, RgB and RgD refer to various substituents as required by the examples.

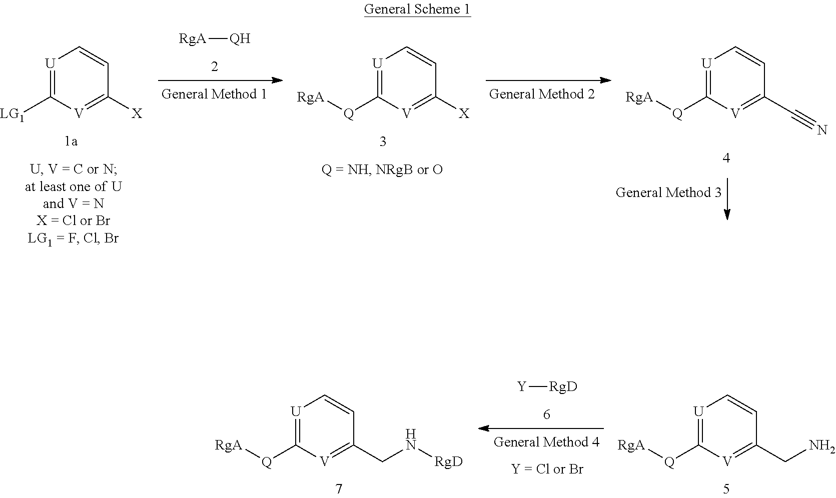

The aryl or heteroaryl halide 1a is reacted under S_{N}Ar conditions (General Method 1) with either an alcohol or amine 2 using an appropriate base, in solvents such as MeCN, 1,4-dioxane, DMF or NMP at elevated temperatures 50-100° C. Alcohols are typically reacted using bases such as caesium carbonate, potassium tert-butoxide or sodium tert-butoxide, whereas amines are typically reacted using bases such as potassium carbonate, caesium carbonate or N,N-diisopropylethylamine. The aryl bromide or chloride 3 can undergo palladium catalysed cyanation using conditions well known in the art (General Method 2); for example by palladium catalysed cyanation with Zn(CN)₂ and Pd(PPh₃)₄ with heating in a solvent such as NMP. The nitrile 4 can be reduced to amine 5 under a variety of standard literature conditions well known in the art (General Method 3); for example under hydrogenation in the presence of Raney Ni, alternatively hydrogenation in the presence of Pd/C, or alternatively with NiCl₂ and NaBH₄ in the presence of Boc₂O, or alternatively with borane. The amine 5 is reacted with aryl bromide or chloride 6 under Buchwald coupling conditions (General Method 4). This Buchwald coupling is carried out for example using BrettPhos Pd G3, BrettPhos Pd G4 or RuPhos Pd G3 catalyst in the presence of a base such a sodium tert-butoxide, caesium carbonate, or potassium hexamethyldisilazide (KHMDS), in a solvent such as 1,4-dioxane or THF. The aryl bromide or chloride 6 can be prepared from readily available starting materials using methods known in the art, or as described herein. Depending on the identity of RgD, a deprotection step (detailed above) may be required to obtain the example compound.

Alternatively, where starting material is commercially available with the nitrile in place, for example 1b in General Scheme 2, it can be reacted under the aforementioned S_{N}Ar conditions (General Method 1) to deliver compound 4.

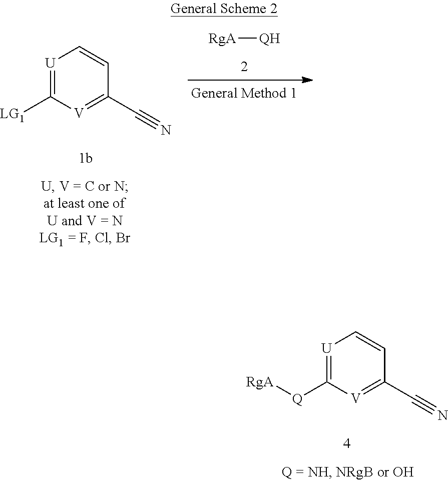

General Schemes 3-5 outline a synthetic route for certain example compounds e.g. those with a 6-membered central ring as defined below, and RgA, RgB, RgD, RgE and RgF refer to various substituents as required by the examples. RgE and RgF may join together to form a ring structure, as required by the examples.

In General Scheme 3 the benzyl halide 8 (where LG=Br or Cl) is reacted with alcohol 2a under typical alkylation conditions (General Method 5, e.g. KOtBu or NaH in DMF, or Cs₂CO₃ or K₂CO₃ in NMP with heating as necessary). Alternatively, a benzyl alcohol 8 (where LG=OH) can be reacted with alcohol 2a under Mitsunobu conditions. Typically the route continues with cyanation, reduction and Buchwald coupling using methods as in General Scheme 1. Depending on the identity of RgD, a deprotection step (detailed above) may be required to obtain the example compound.

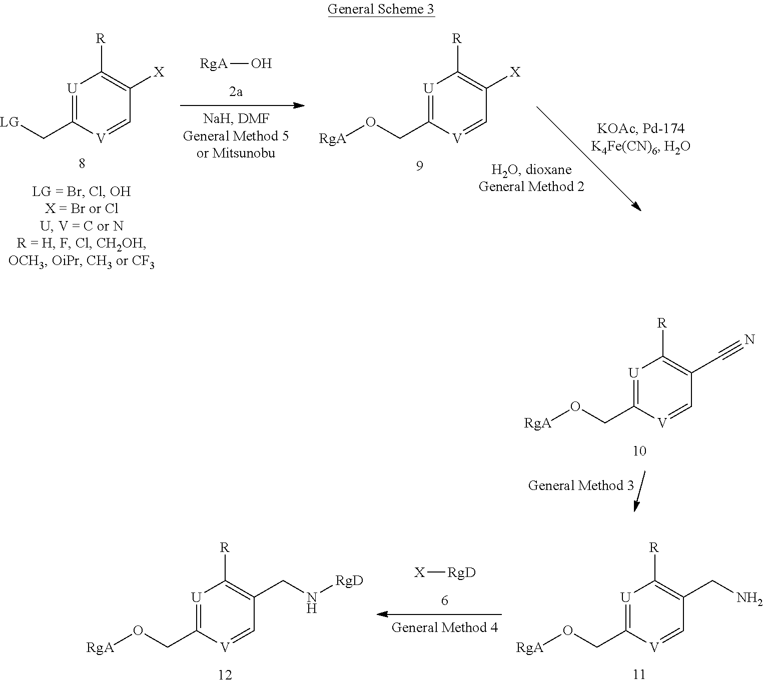

Alternatively, for example as shown in General Scheme 4, where starting material is available with the nitrile already in place, for example compound 13, the amine can be prepared by reduction of the nitrile using General Method 3. The amine may be protected in a stepwise fashion with a protecting group such as a carbamate, for example tert-butoxy carbamate, resulting in the tert-butoxy carbamate 14. It is also possible, as shown in General Scheme 4, to carry out an in situ protection of the amine group (for example according to General Methods 3d or 3e). Protection of the amine group may be helpful to enable, for example, purification by chromatography of the intermediate compound 14. Protection of the amine also facilitates subsequent synthetic steps. Thus, according to General Method 5, compound 14 can be reacted directly with alcohol 2 under Mitsunobu conditions in the presence of PPh₃. Alternatively, a suitable leaving group, such as halide or mesylate, can be generated using conditions well known in the art such as, for example; chlorination via a mesylate, bromination with PBr₃, or bromination with CBr₄ and PPh₃, using a suitable solvent such as DCM, THF or CCl₄(General Method 6), to give compound 15. An alkylation (General Method 5, e.g. KO^{t}Bu or NaH in DMF, or Cs₂CO₃ or K₂CO₃ in NMP with heating as necessary) can then be carried out. The tert-butoxy carbamate protecting group is removed from intermediate 16 using standard conditions such as TFA, or HCl in 1,4-dioxane (General Method 7). Finally, Buchwald coupling (General Method 4) completes the route.

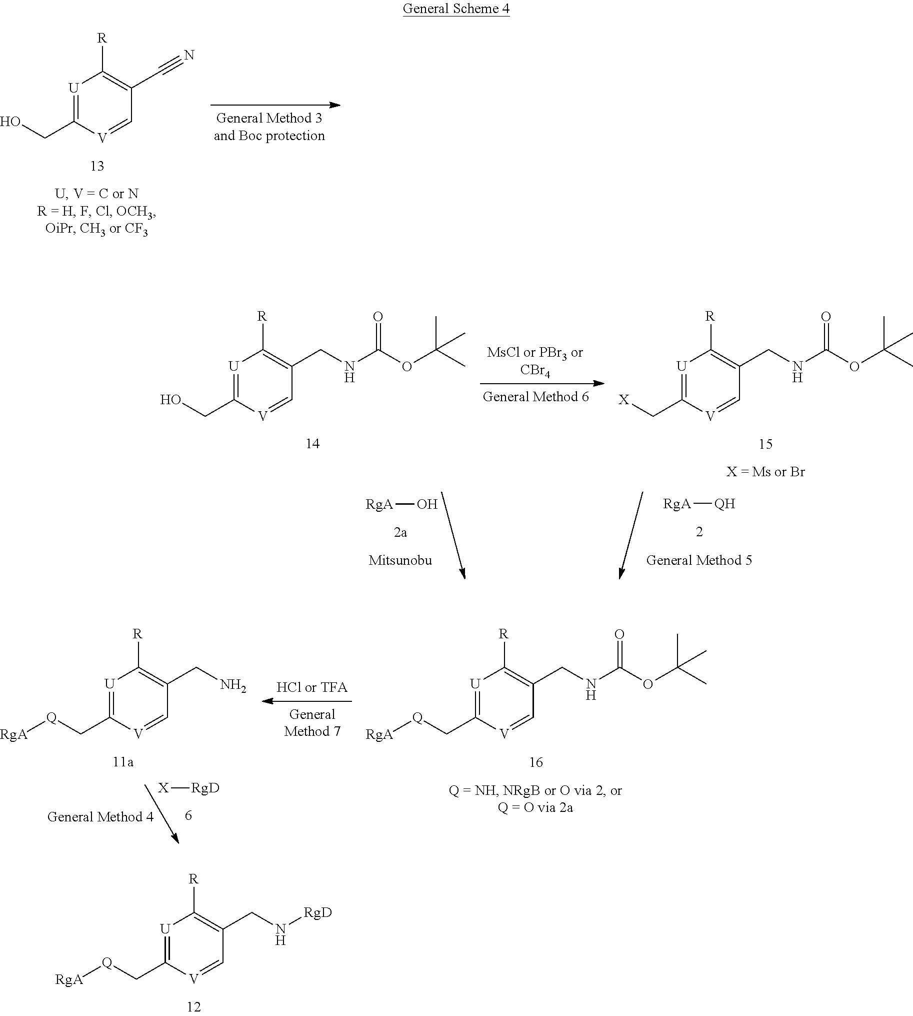

Other analogues of compound 11, such as compound 11b and 11c, can be synthesised according to General Scheme 5.

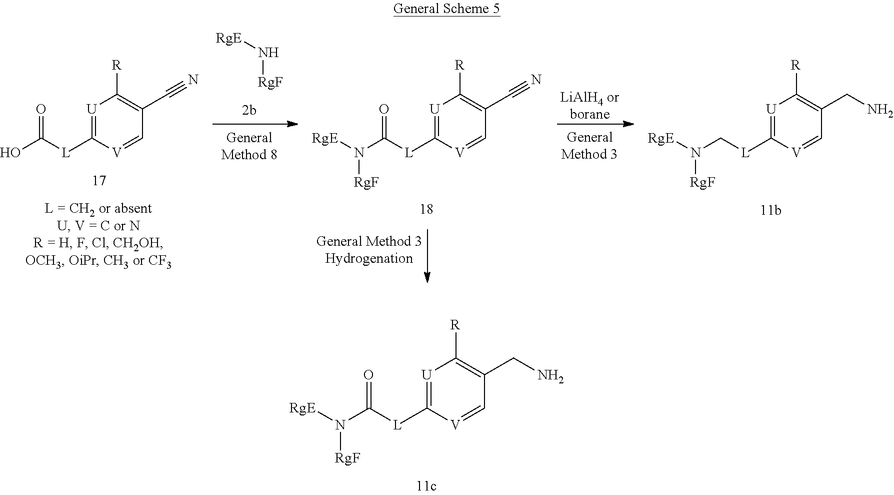

Amide coupling (General Method 8) using conditions well known in the art, for example using HATU, is carried out to form amide 18. A global reduction is then possible, reducing both the amide and the nitrile in a single step, using for example LiAlH₄ or borane in THF to give 11b. Alternatively the nitrile can be reduced under hydrogenation conditions (General Method 3) leaving the amide intact to give compound 11c.

In certain example compounds e.g. those where RgA, RgE or RgF contains a tertiary amine, this tertiary amine can be formed before (General Scheme 6) or during (General Scheme 7) the general routes.

An amine such as compound 2c which is purchased or synthesised, can be reacted following the route and General Methods as illustrated by General Scheme 6.

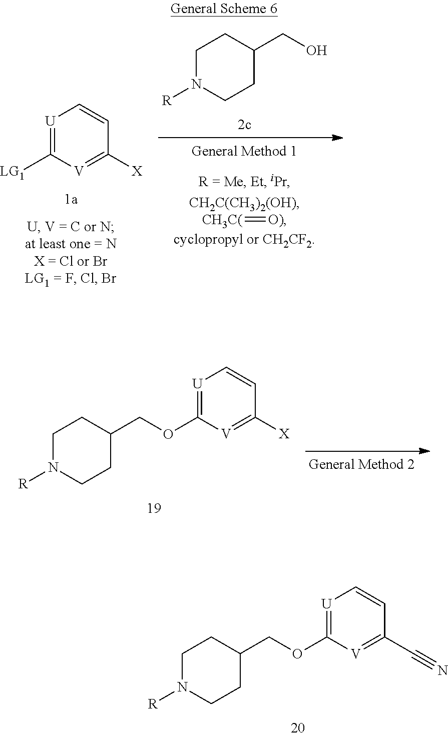

Alternatively, the a primary or secondary amine can be protected with standard protecting groups, for example tert-butoxy carbamate, as shown in the carbamate 2d (General Scheme 7) and manipulated before a nitrile reduction step (General Method 3).

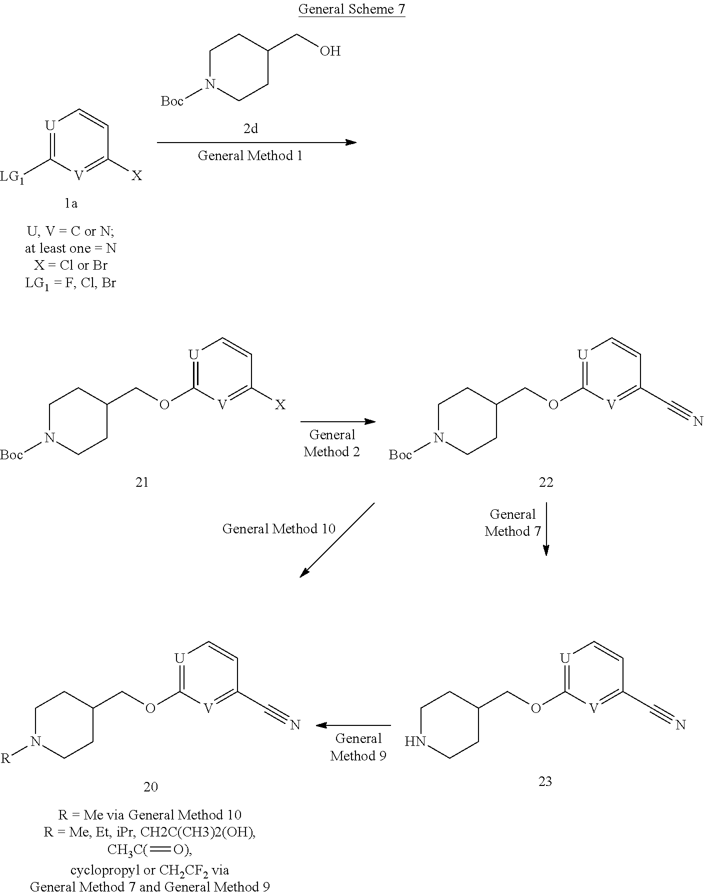

Compound 2d can undergo alkylation (General Method 1) and cyanation (General Method 2) to form compound 22. The amine can then be deprotected and alkylated, either sequentially by deprotection with acid (General Method 7, e.g. HCl or TFA) followed by reductive alkylation (General Method 9), or in a one-pot tandem Eshweiler Clarke reaction (General Method 10).

General Schemes 8-10 outline a synthetic route for certain example compounds e.g. those with a 5-membered central ring as defined below, and RgD, RgG and RgH refer to various substituents as required by the examples.

Compounds with an N-substituted 5-membered central ring can be synthesised according to the general route outlined in General Scheme 8.

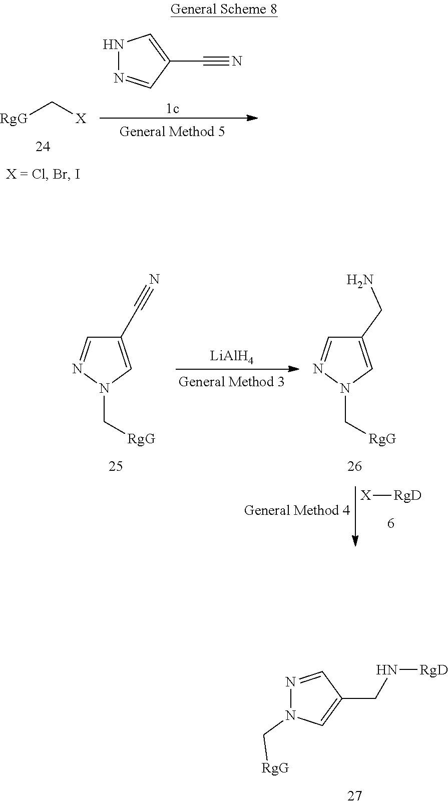

The alkyl halide 24 is reacted with heterocycle 1c under general alkylation conditions for such a transformation, using bases such as K₂CO₃ or Cs₂CO₃, in solvents such as MeCN, 1,4-dioxane, DMF or NMP, at elevated temperature or under microwave conditions as necessary (General Method 5). The nitrile 25 is reduced to amine 26 using General Method 3, for example with LiAlH4, which is then reacted under Buchwald conditions with aryl bromide or chloride 6 (General Method 4). Depending on the identity of RgD, a deprotection step (detailed above) may be required to obtain the example compound. In certain example compounds e.g. those where RgG contains a tertiary amine, this tertiary amine can be formed before or manipulated during the general routes as described previously, e.g. General Schemes 6 and 7.

In General Scheme 9, the heteroaryl halide 28a is reacted under S_{N}Ar conditions (General Method 1) with, for example, an alcohol (exemplified in General Scheme 9 with compound 2e) using an appropriate base such as caesium carbonate, potassium tert-butoxide or sodium tert-butoxide, in solvents such as MeCN, 1,4-dioxane, DMF or NMP at elevated temperatures 50-100° C. as necessary to provide ether 29. The synthesis is completed via cyanation (General Method 2), reduction (hydrogenation, General Method 3) and Buchwald coupling (General Method 4) as described previously, e.g. General Schemes 1 and 3).

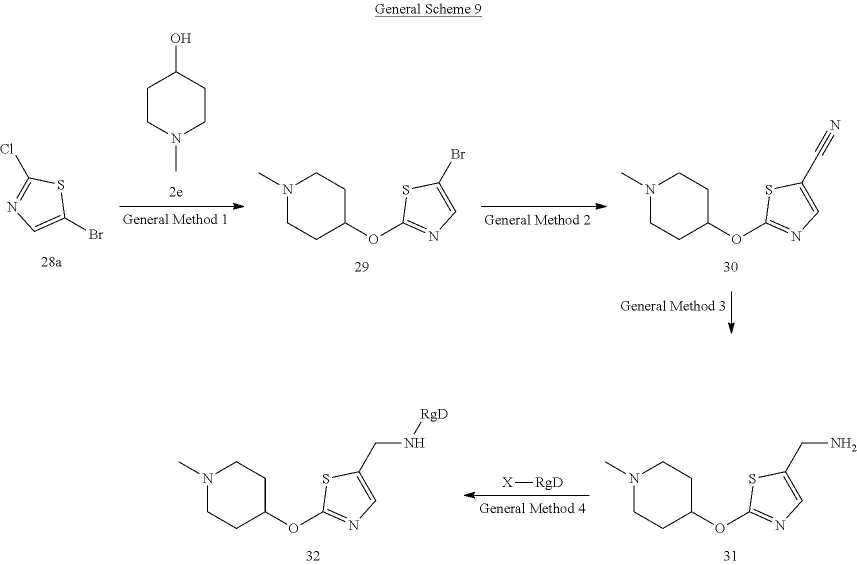

General Scheme 10 outlines a synthetic route for certain example compounds e.g. those with a 5-membered central ring as defined below.

The heteroaryl 28b can be brominated using conditions well known in the art such as, for example, with N-bromosuccinimide (NBS) using a suitable solvent such as CCl₄(General Method 6), to give bromide 33. An alkylation (General Method 5, e.g. KO^{t}Bu or NaH in DMF, or Cs₂CO₃ or K₂CO₃ in NMP with heating as necessary) can then be carried out to afford compound 34, followed by reduction (hydrogenation, General Method 3) and Buchwald coupling (General Method 4) as described previously, e.g. in General Schemes 1 and 3).

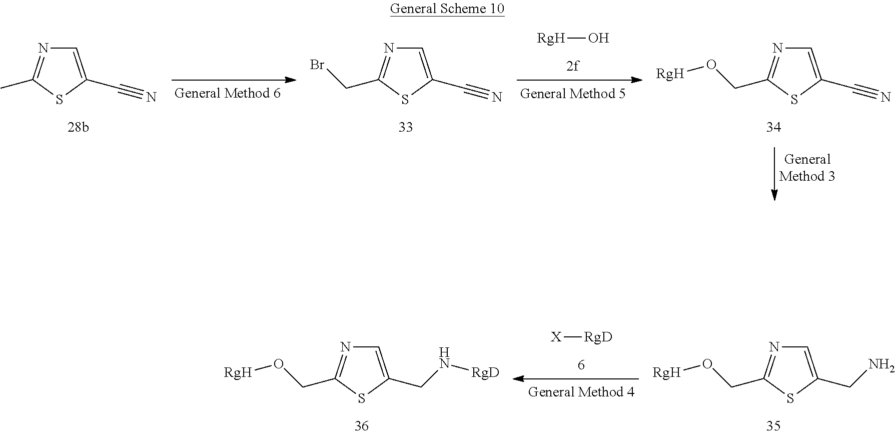

Gem-dimethyl, cyclopropyl and cyclobutyl groups can be accessed from the appropriate nitrile using literature methods as shown in General Schemes 11-14. RgB, RgJ, RgK, RgL and RgM refer to various substituents as required by the example compounds described herein.

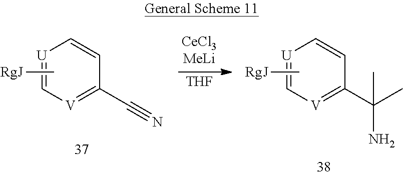

For example, the nitrile 37 can be reacted with methyl lithium at −78° C. in the presence of cerium (III) chloride in a solvent such as THF or 1,4-dioxane to form the gem-dimethyl amine 38 (General Scheme 11).

There are several literature conditions to form cyclopropyl amines from aromatic nitriles in the presence of titanium alkoxides. For example, an aromatic nitrile 39 can be reacted at −70° C. with titanium isopropoxide and ethylmagnesium bromide followed by addition of a Lewis acid such as boron trifluoride etherate (J. Org. Chem. 2003, 68, 18, 7133-7136) to provide the cyclopropyl amine 40 (General Scheme 12). Alternatively, cyclopropyl amine 40 can be formed by the addition of diethyl zinc in the presence of MeTi(OⁱPr)₃, LiOⁱ Pr, Lil in THF, rt (Org. Lett. 2003, 5, 5, 753-755) to aromatic nitrile 39.

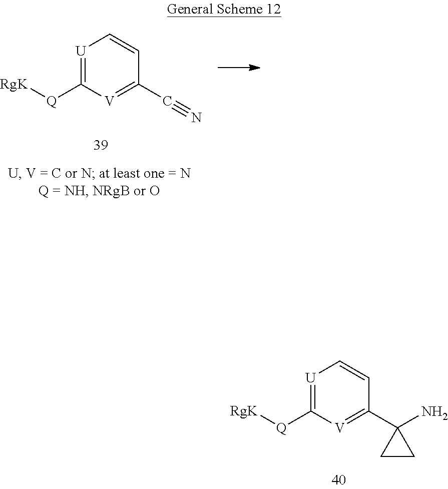

A cyclobutyl group can also be synthesised by methods reported in the literature and outlined in General Schemes 13-14.

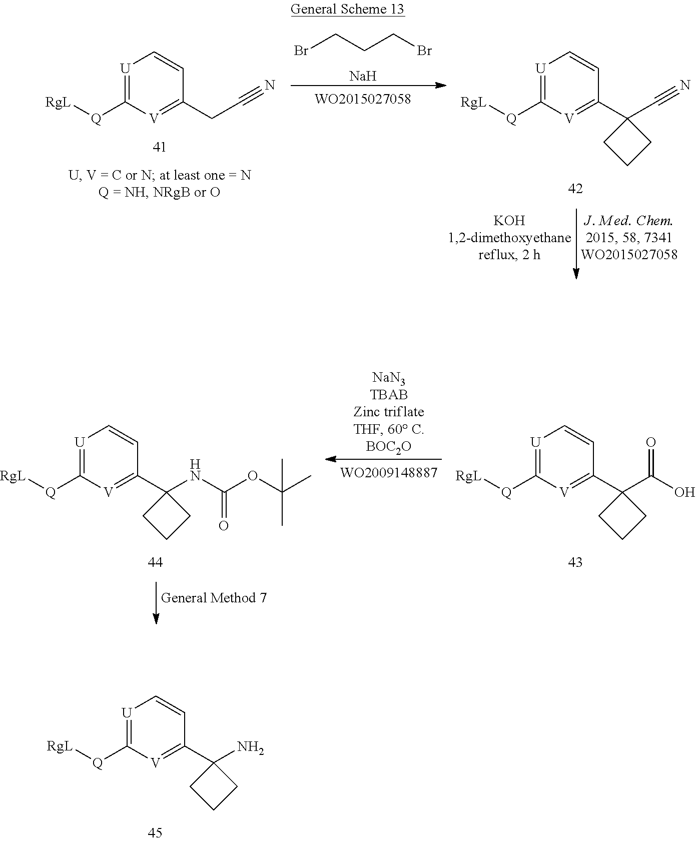

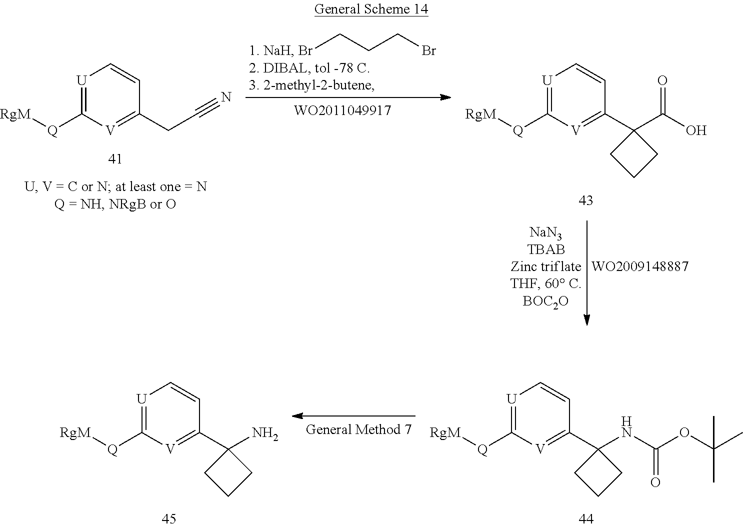

General Scheme 15 outlines a synthesis of example compounds described herein via an alkyne e.g. to provide compounds with a —CH₂CH₂— linker. For example, fluoropyridine 46 can be reacted using the standard S_{N}Ar conditions (for example with base Cs₂CO₃, General Method 1). The alkyne 47 can then be reacted with heteroarylbromide 48 under a palladium catalysed Sonogashira coupling. The alkyne 49 can be reduced by hydrogenation (General Method 3). RgA refers to various substituents as required by the examples.

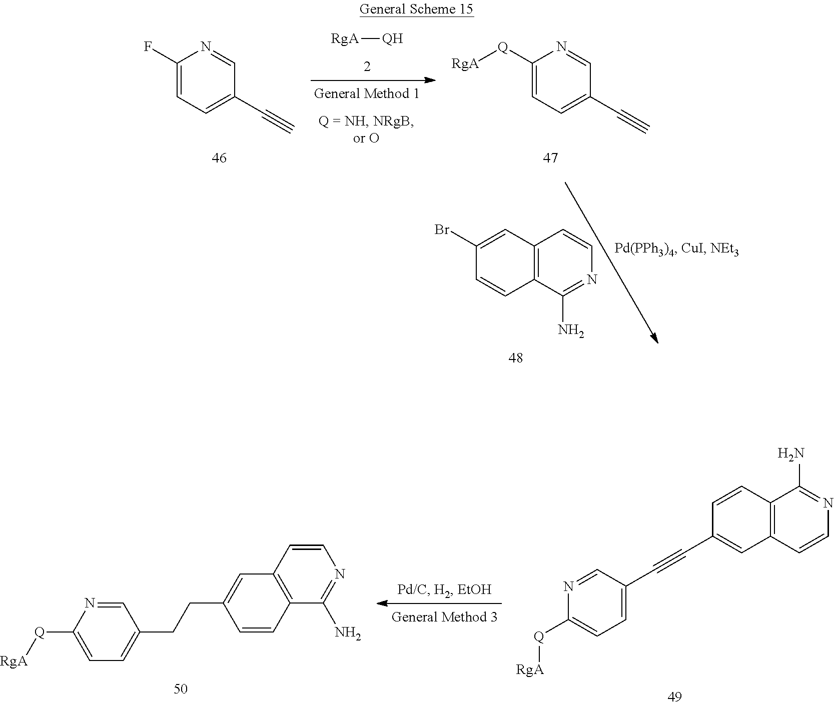

A Simmons Smith cyclopropanation may be utilised, via an alkene 51, as illustrated in General Scheme 16 to form a cyclopropyl ring 52. RgB, RgD and RgN refer to various substituents as required by the example compounds.

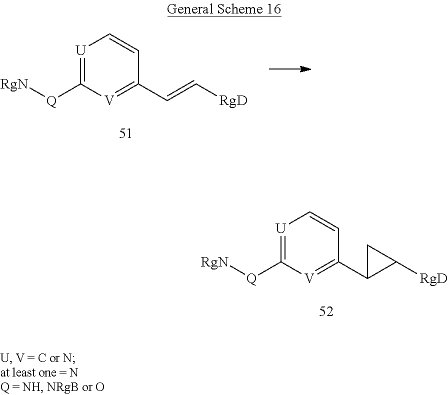

The aforementioned General Methods, for example as outlined in General Scheme 17 below, provide a synthesis of example compounds that have e.g. a —CH₂O— ether linker. These examples can be accessed via an alcohol, for example by taking protected alcohol through the synthesis. The final step to convert benzyl alcohol 56 to ether 57 typically requires reaction with a strong base such as NaOtBu in NMP at elevated temperature or in a microwave reactor. RgP refers to various substituents as required by the example compounds.

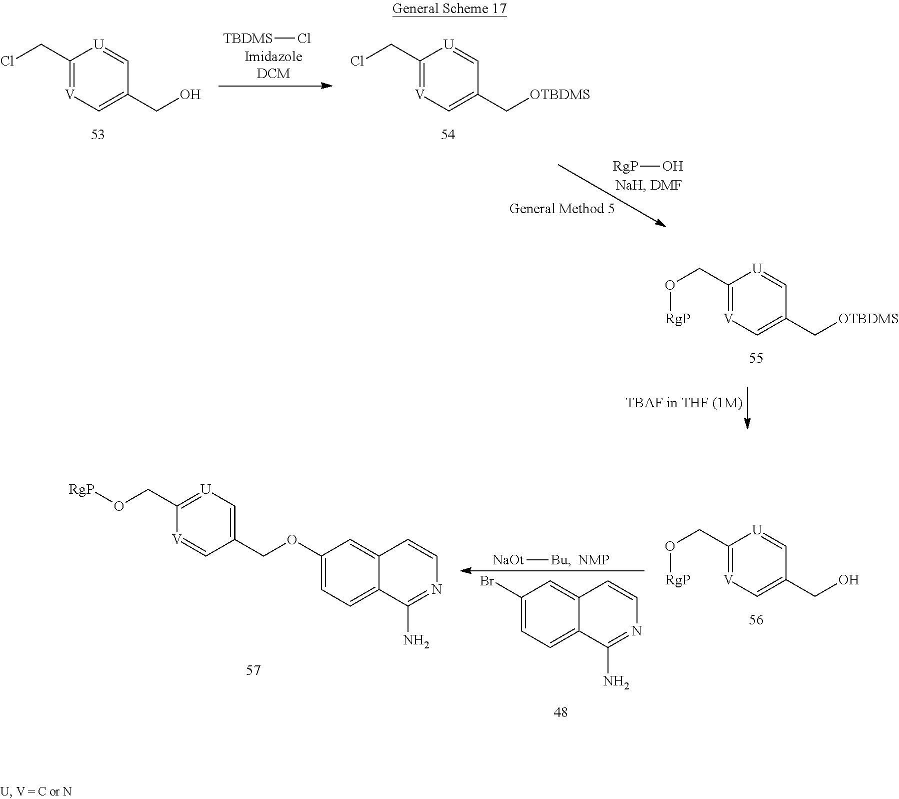

Alternatively, an alcohol 60 may be synthesised from an aryl bromide 58, via carbonylation and reduction as outlined in General Scheme 18. The final step to convert alcohol 60 to ether 61 typically requires reaction with a strong base such as NaOtBu in NMP at elevated temperature or in a microwave reactor. RgS refers to various substituents as required by the example compounds.

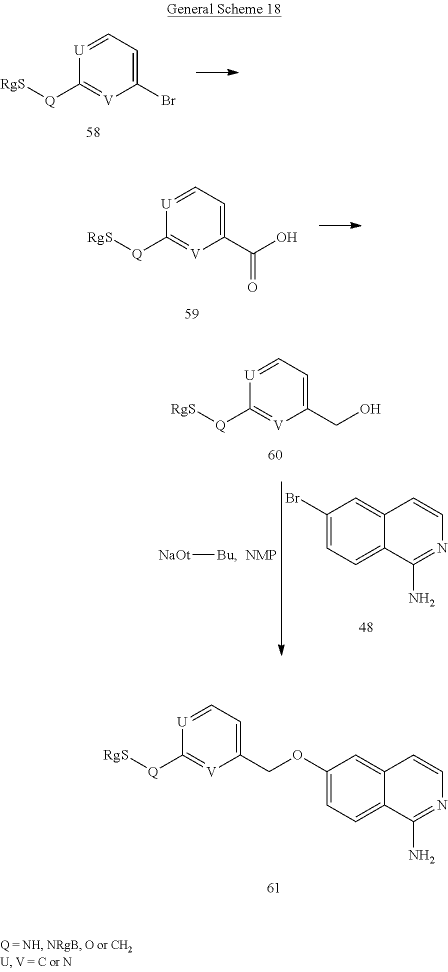

In example compounds described herein containing a primary or secondary amine, a protecting group strategy may be required. Alternative protecting groups can be used with different deprotection conditions such than an orthogonal protecting group strategy can be applied. For example, compounds defined herein containing a 6,6 ring system, as shown in General Scheme 19, a protected amine can be installed by reaction of chloride 63 with 2,4-dimethoxybenylamine using General Method 1, for example using basic conditions such as potassium carbonate or pyridine in a solvent such as NMP, either thermally and under microwave conditions. RgT refers to various substituents as required by the example compounds.

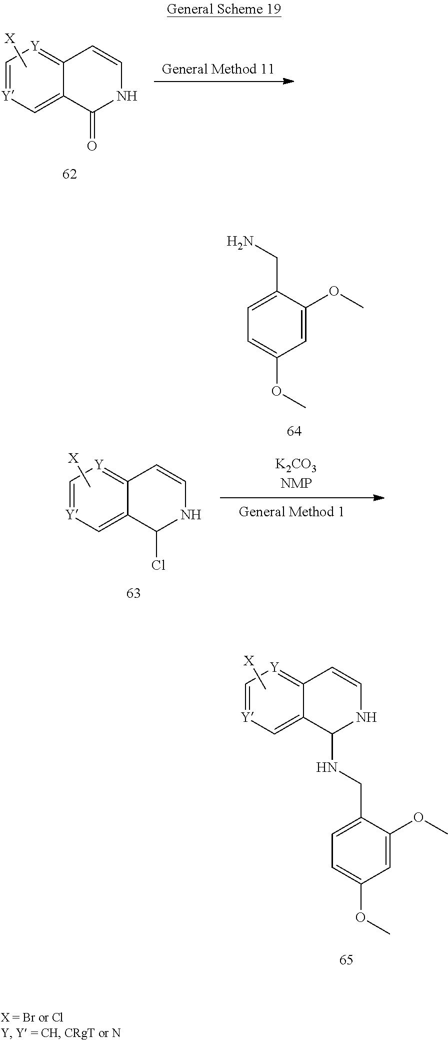

Typically, at the end of the synthetic sequence, the 2,4-dimethoxybenyl protecting group is removed using undiluted TFA at 50° C. (General Scheme 20). RgT, RjA and RjB refer to various substituents as required by the examples.

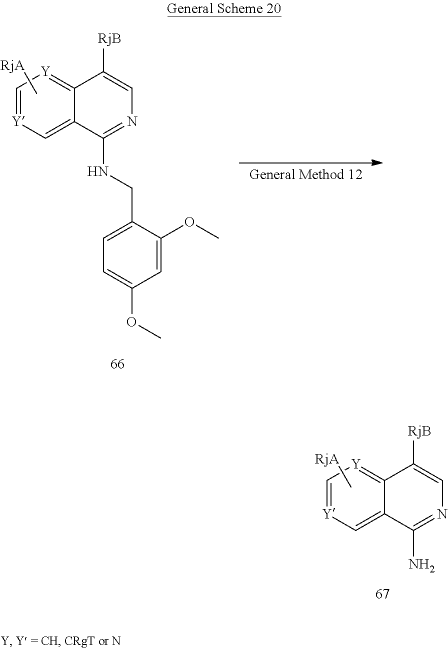

Alternatively, when starting materials are available with the amine already installed, a carbamate protecting group can be used. For example, as outlined in General Scheme 21, the amine is reacted with methyl chloroformate under basic conditions with organic bases such as TEA or DIPEA in a solvent such as DCM to afford the methyl carbamate 69. RgC refers to various substituents as required by the examples.

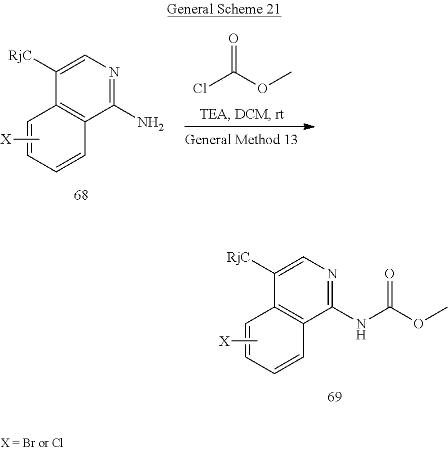

Typically at the end of the synthetic sequence the methyl carbamate protecting group is deprotected using basic conditions, such as KOH or LiOH in solvents such as 1,4-dioxane, MeCN, THF and optionally 10% water, at elevated temperature, typically 50° C. (General Scheme 22). RjC and RjD refer to various substituents as required by the examples.

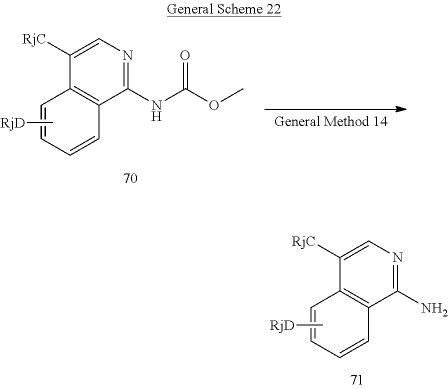

Another protecting group that may be used where example compounds described herein contain a 6,6 ring system is boc. Also, especially where for example, example compounds described herein contain a 5,6 ring system, SEM, boc and sulphonyl protecting groups may typically be used. Protecting groups may subsequently be deprotected using standard literature procedures, for example those described by T. W. Greene and P. G. M. Wuts in “Protective groups in organic chemistry” John Wiley and Sons, 4^{th} Edition, 2006.

An example of the installation of a SEM protecting group is shown in General Scheme 23 whereby the indole 72 is treated with a base such as NaH in a solvent such as DMF, followed by addition of 2-(trimethylsilyl)ethoxymethyl chloride (General Method 15).

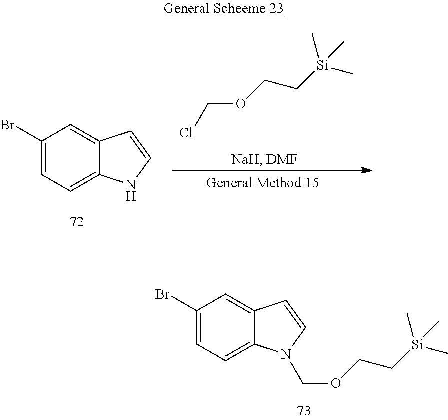

### Synthesis of Intermediates

### Intermediate 1

### (5,6,7,8-Tetrahydroimidazo[1,2-a]pyridin-7-yl)methanol

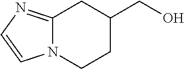 [SMILES: OCC1CCn2ccnc2C1]

### Methyl imidazo[1,2-a]pyridine-7-carboxylate

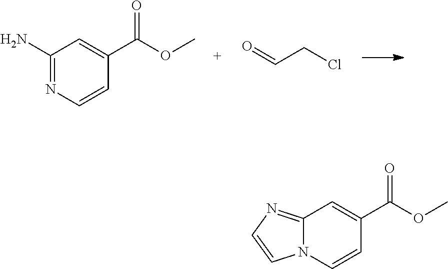

To a stirred suspension of methyl 2-aminoisonicotinate (11.0 g, 0.72 mol) and NaHCO₃ (12 g, 0.14 mol) in EtOH (30 mL) was added 2-chloroacetaldehyde (50% in water) (14 mL, 0.11 mol) and the resultant suspension heated to 80° C. for 5 h. The reaction mixture was cooled and concentrated. The resultant solid was partitioned between water (50 mL) and DCM (50 mL), passed through a phase separator and concentrated to give the product (13 g, 93% yield) as an orange solid. [M+H]⁺=177.3

¹H NMR (500 MHz, DMSO-d6) δ 3.90 (3H, s), 7.35 (1H, dd, J=7.1, 1.7 Hz), 7.82 (1H, d, J=1.1 Hz), 8.17 (2H, m), 8.67 (1H, dd, J=7.1, 0.9 Hz)

### Methyl 5,6,7,8-tetrahydroimidazo[1,2-a]pyridine-7-carboxylate

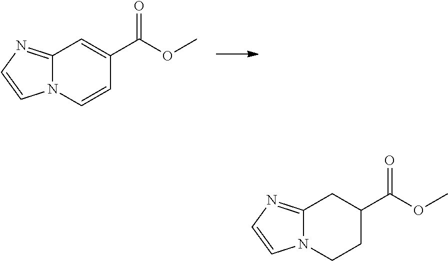

Hydrogenation of methyl imidazo[1,2-a]pyridine-7-carboxylate (7.3 g, 41 mmol) was completed using General Method 3e, in the presence of 12M HCl aq. (3.5 mL, 41 mmol) in EtOH (90 mL), under 5 bar H₂ at 80° C. for 1 h. The crude reaction mixture was taken up in sat. NaHCO₃ (100 mL) which was extracted with DCM (2×100 mL). The organics were collected and concentrated to give the product (7.0 g, 71% yield) as a brown oil M+H]⁺=181.2

### (5,6,7,8-Tetrahydroimidazo[1,2-a]pyridin-7-yl)methanol

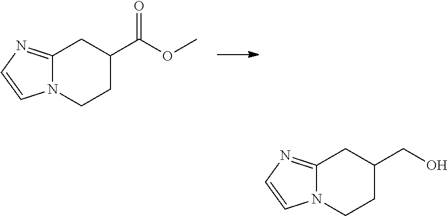

Reduction of methyl 5,6,7,8-tetrahydroimidazo[1,2-a]pyridine-7-carboxylate (5.25 g, 29.1 mmol) was carried out using General Method 3b over 1 h. The reaction mixture was filtered through Celite® and the filtrate was concentrated to yield the product (3.8 g, 83% yield) as a brown oil. [M+H]⁺=153.1

### Intermediate 2

### (3-Methyl-5,6,7,8-tetrahydroimidazo[1,2-a]pyridin-7-yl)methanol

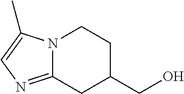

### Methyl 3-methylimidazo[1,2-a]pyridine-7-carboxylate

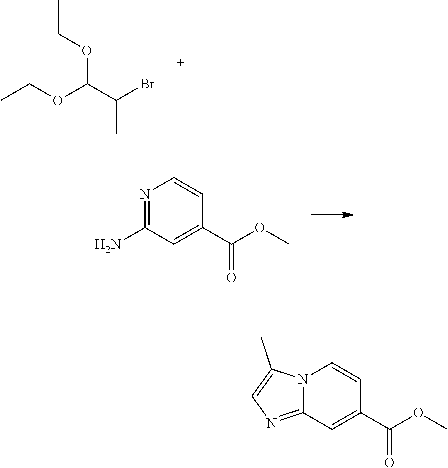

A mixture of 2-bromo-1,1-diethoxypropane (2081 mg, 9.86 mmol) and 2M HCl (4.9 mL, 9.86 mmol) was heated to 90° C. and stirred for 60 min. The reaction solution was cooled to rt and neutralized with Na₂CO₃ (828 mg, 9.86 mmol). Methyl 2-aminopyridine-4-carboxylate (1000 mg, 6.57 mmol) and MeOH (7 mL) were added successively and the reaction heated to 90° C. for 18 h. The solution was concentrated and purified by flash chromatography (silica, 30-100% EtOAc in Pet. Ether followed by 0-20% MeOH in EtOAc) to give the product (463 mg, 37% yield) as an off-white solid. [M+H]⁺=191.0

¹H NMR (DMSO, 400 MHz) δ 2.51-2.53 (3H, m), 3.90 (3H, s), 7.35 (1H, dd, J=7.2, 1.7 Hz), 7.62 (1H, d, J=1.0 Hz), 8.13 (1H, dd, J=1.7, 1.0 Hz), 8.37 (1H, dd, J=7.2, 1.0 Hz)

### Methyl 3-methyl-5,6,7,8-tetrahydroimidazo[1,2-a]pyridine-7-carboxylate

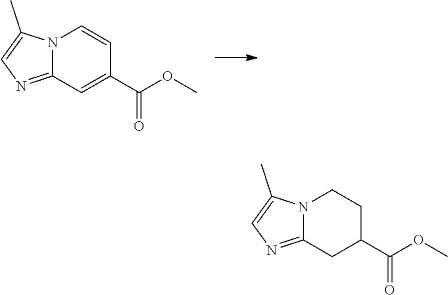

Methyl 3-methylimidazo[1,2-a]pyridine-7-carboxylate (463 mg, 2.43 mmol) was reacted following General Method 3e. The solvent was removed to afford the product (441 mg, 93% yield) as a colourless oil.

[M+H]⁺=195.1

¹H NMR (CDCl₃, 400 MHz) δ 2.04-2.13 (1H, m), 2.14 (3H, d, J=1.1 Hz), 2.31-2.45 (1H, m), 2.81-2.91 (1H, m), 2.99 (1H, dd, J=16.5, 10.2 Hz), 3.19 (1H, ddd, J=16.4, 5.4, 1.5 Hz), 3.64-3.72 (1H, m), 3.74 (3H, s), 3.87-4.00 (1H, m), 6.69 (1H, d, J=1.1 Hz)

### (3-Methyl-5,6,7,8-tetrahydroimidazo[1,2-a]pyridin-7-yl)methanol

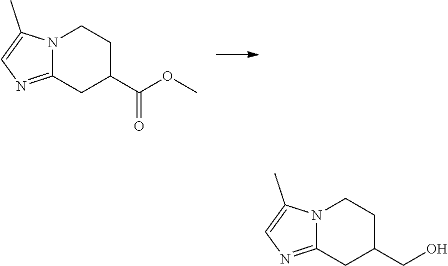

Reduction of the ester methyl 3-methyl-5,6,7,8-tetrahydroimidazo[1,2-a]pyridine-7-carboxylate (441 mg, 2.27 mmol) was performed using General Method 3b over 70 min. The product was isolated (227 mg, 60% yield) as a white solid and used without further purification.

[M+H]⁺=167.0

¹H NMR (CDCl₃, 400 MHz) δ 1.65-1.79 (1H, m), 2.00-2.29 (6H, m), 2.50 (1H, dd, J=16.5, 10.7 Hz), 3.01 (1H, ddd, J=16.4, 5.1, 1.6 Hz), 3.59-3.74 (3H, m), 3.84-3.96 (1H, m), 6.66 (1H, s)

### Intermediate 3 (2-Methyl-5,6,7,8-tetrahydroimidazo[1,2-a]pyridin-7-yl)methanol

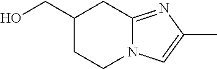

### Methyl 2-methylimidazo[1,2-a]pyridine-7-carboxylate

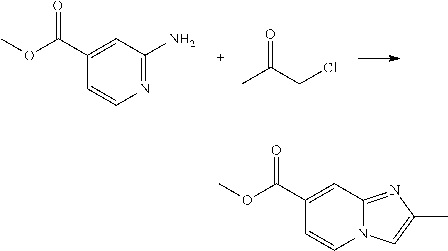

Methyl 2-aminopyridine-4-carboxylate (2.0 g, 13.14 mmol) was dissolved in EtOH (20 mL) and 1-chloropropan-2-one (3.6 g, 39.43 mmol) and Na₂CO₃ (2.80 g, 32.86 mmol) were added. The suspension was stirred for 48 h at 80° C. The reaction mixture was cooled to rt, concentrated and the resulting residue was purified by flash chromatography (Silica, 20-100% EtOAc in Pet. Ether followed by 0-20% MeOH in EtOAc) to afford the product (755 mg, 30% yield) as a brown solid.

[M+H]⁺=191.0

### Methyl 2-methyl-5,6,7,8-tetrahydroimidazo[1,2-a]pyridine-7-carboxylate

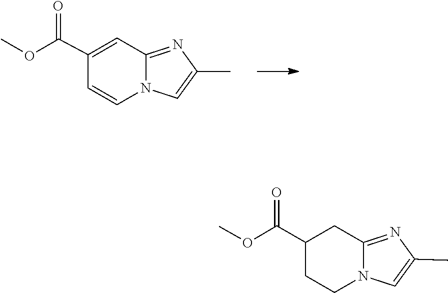

Methyl 2-methylimidazo[1,2-a]pyridine-7-carboxylate (443 mg, 2.33 mmol) was semi-saturated following General Method 3e for 45 min, at 70° C., using a 10% Pd/C CatCart. The solvent was removed in vacuo to afford the product (376 mg, 83% yield) as a pale yellow oil.

[M+H]⁺=195.1

### (2-Methyl-5,6,7,8-tetrahydroimidazo[1,2-a]pyridin-7-yl)methanol

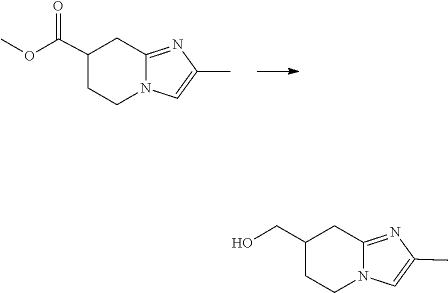

The ester, methyl 2-methyl-5,6,7,8-tetrahydroimidazo[1,2-a]pyridine-7-carboxylate (376 mg, 1.94 mmol) was reduced using General Method 3b over 90 min. The product was isolated (318 mg, 99% yield) as a colourless oil.

[M+H]⁺=167.0

¹H NMR (CDCl₃, 400 MHz) δ 1.65-1.76 (1H, m), 2.05-2.16 (2H, m), 2.17 (3H, d, J=1.0 Hz), 2.42-2.52 (1H, m), 2.98 (1H, ddd, J=16.6, 5.0, 1.5 Hz), 3.58-3.71 (3H, m), 3.77-3.87 (1H, m), 3.99 (1H, ddd, J=12.4, 5.6, 2.9 Hz), 6.48 (1H, t, J=1.1 Hz)

### Intermediate 4

### (4-((1-Methylpiperidin-4-yl)oxy)phenyl)methanamine

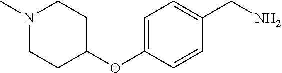 [SMILES: CN1CCC(Oc2ccc(CN)cc2)CC1]

### 4-((1-Methylpiperidin-4-yl)oxy)benzonitrile

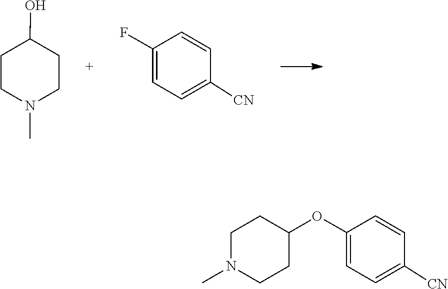

Following General Method 1a, 1-methylpiperidin-4-ol (0.95 g, 8.25 mmol) was reacted with 4-fluorobenzonitrile (1.00 g, 8.26 mmol. The crude product was purified by flash chromatography (Silica, 0-10% MeOH in DCM) to obtain the product (1.60 g, 87% yield) as a white solid.

[M+H]⁺=217.1

¹H NMR (500 MHz, DMSO-d6) δ 1.58-1.69 (2H, m), 1.89-1.96 (2H, m), 2.14-2.21 (5H, m), 2.56-2.65 (2H, m), 4.51 (1H, tt, J=8.6, 4.1 Hz), 7.09-7.15 (2H, m), 7.70-7.76 (2H, m).

### (4-((1-Methylpiperidin-4-yl)oxy)phenyl)methanamine

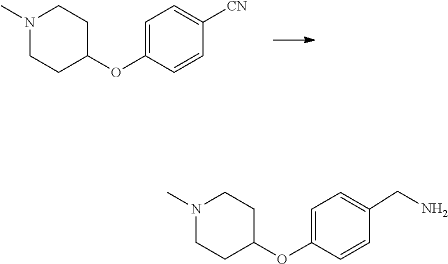 [SMILES: CN1CCC(Oc2ccc(C#N)cc2)CC1 ; CN1CCC(Oc2ccc(CN)cc2)CC1]

Nitrile reduction of 4-((1-methylpiperidin-4-yl)oxy)benzonitrile (1.59 g, 7.35 mmol) was performed following General Method 3e using 10% Pd/C (160 mg, 1.50 mmol) and sulfuric acid (1.6 mL, 30.02 mmol) in EtOH (25 mL) under 3 bar of H₂ at rt for 64 h. The crude product was basified to pH 10 with sat. Na₂CO₃ (aq) while cooling in an ice/water bath then with NaOH (2 M) to pH 14. The aqueous layer was extracted with EtOAc (3×50 mL), DCM (2×40 mL) and THF (40 mL). The combined organic layers were dried (MgSO₄), filtered and concentrated to obtain the product (820 mg, 43% yield) as a yellow oil which was taken onto the next step without further purification.

[M+H]⁺=221.1

¹H NMR (500 MHz, DMSO-d6) δ 1.55-1.64 (2H, m), 1.71 (2H, br. s), 1.85-1.93 (2H, m), 2.10-2.20 (5H, m), 2.55-2.64 (2H, m), 3.62 (2H, s), 4.30 (1H, tt, J=8.2, 4.0 Hz), 6.84-6.88 (2H, m), 7.18-7.23 (2H, m)

### Intermediate 5

### (4-(((1-Methylpiperidin-4-yl)oxy)methyl)phenyl)methanamine

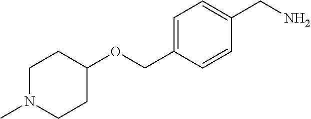 [SMILES: CN1CCC(OCc2ccc(CN)cc2)CC1]

### Tert-butyl 4-((4-cyanobenzyl)oxy)piperidine-1-carboxylate

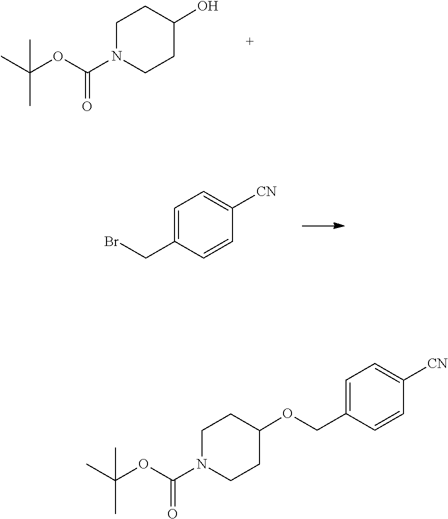 [SMILES: CC(C)(C)OC(=O)N1CCC(O)CC1 ; N#Cc1ccc(CBr)cc1 ; CC(C)(C)OC(=O)N1CCC(OCc2ccc(C#N)cc2)CC1]

Following General Method 5a, tert-butyl 4-hydroxypiperidine-1-carboxylate (1.76 g, 8.67 mmol) was reacted with 4-(bromomethyl)benzonitrile (1.7 g, 8.67 mmol) in the presence of NaH (60% wt. on mineral oil) (0.35 g, 8.75 mmol) for 18 h. The crude product was purified by flash chromatography (Silica, 0-50% EtOAc in isohexane) to afford the product (1.65 g, 57% yield) as a colourless gum which set on standing.

[M+H]⁺=261.1

¹H NMR (500 MHz, DMSO-d6) δ 1.40 (9H, s), 1.40-1.46 (2H, m), 1.80-1.86 (2H, m), 3.00-3.10 (2H, m), 3.55-3.67 (3H, m), 4.62 (2H, s), 7.52-7.55 (2H, m), 7.80-7.83 (2H, m).

### 4-(((1-Methyl piperidin-4-yl)oxy)methyl)benzonitrile

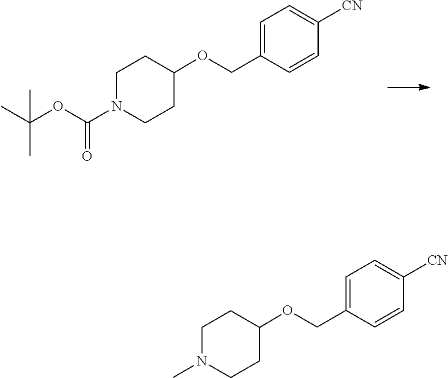 [SMILES: CC(C)(C)OC(=O)N1CCC(OCc2ccc(C#N)cc2)CC1 ; CN1CCC(OCc2ccc(C#N)cc2)CC1]

Following General Method 10, tert-butyl 4-((4-cyanobenzyl)oxy)piperidine-1-carboxylate (1.60 g, 5.06 mmol) in formic acid (2.0 mL, 52.1 mmol) was reacted with formaldehyde (37% in water) (0.80 mL, 11.0 mmol) at 90° C. for 2 h. The reaction mixture was concentrated and the crude product was purified by flash chromatography (Silica, 0-20% (0.7 M NH₃ in MeOH) in DCM) to afford the product (980 mg, 82% yield) as a colourless oil.

¹H NMR (500 MHz, DMSO-d6) δ 1.47-1.56 (2H, m), 1.82-1.89 (2H, m), 1.97-2.05 (2H, m), 2.13 (3H, s), 2.55-2.63 (2H, m), 3.38 (1H, tt, J=8.6, 4.1 Hz), 4.59 (2H, s), 7.51-7.54 (2H, m), 7.80-7.83 (2H, m).

### (4-(((1-Methylpiperidin-4-yl)oxy)methyl)phenyl)methanamine

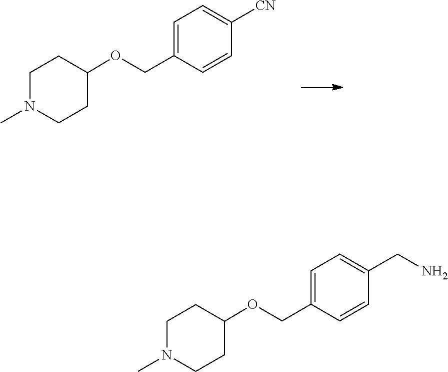 [SMILES: CN1CCC(OCc2ccc(C#N)cc2)CC1 ; CN1CCC(OCc2ccc(CN)cc2)CC1]

The nitrile 4-(((1-methylpiperidin-4-yl)oxy)methyl)benzonitrile (380 mg, 1.65 mmol) was reduced according to General Method 3b, for 18 h. The product (380 mg, 93% yield) was isolated as a colourless solid and used without further purification.

[M+H]⁺=235.4

¹H NMR (500 MHz, DMSO-d6) δ 1.43-1.54 (2H, m), 1.74-1.87 (2H, m), 1.94-2.01 (2H, m), 2.12 (3H, s), 2.55-2.62 (2H, m), 3.38-3.43 (1H, m), 3.69 (2H, d, J=4.1 Hz), 4.45 (2H, s), 7.22-7.25 (2H, m), 7.27-7.30 (2H, m). NH₂ not observed.

### Intermediate 6

### (2-Fluoro-4-(((1-methylpiperidin-4-yl)oxy)methyl)phenyl)methanamine

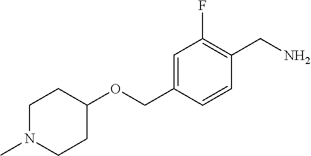 [SMILES: CN1CCC(OCc2ccc(CN)c(F)c2)CC1]

### Tert-butyl 4-((4-bromo-3-fluorobenzyl)oxy)piperidine-1-carboxylate

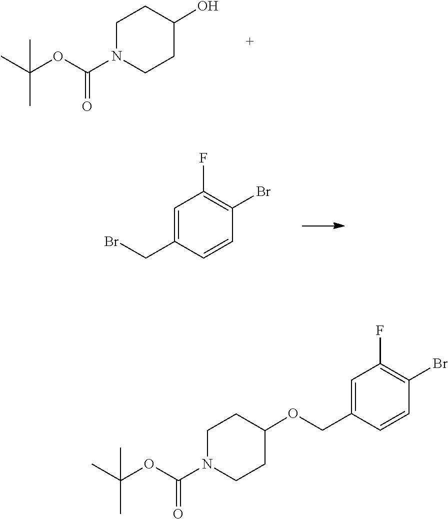 [SMILES: CC(C)(C)OC(=O)N1CCC(O)CC1 ; Fc1cc(CBr)ccc1Br ; CC(C)(C)OC(=O)N1CCC(OCc2ccc(Br)c(F)c2)CC1]

Using General Method 5a, tert-butyl 4-hydroxypiperidine-1-carboxylate (1.76 g, 8.67 mmol) was reacted with 1-bromo-4-(bromomethyl)-2-fluorobenzene (1.7 g, 8.67 mmol) at rt for 2 h. Sat. NaHCO₃ (aq) (100 mL) was added then the reaction mixture was extracted with TBME (2×100 mL). The organic phases were combined, dried (MgSO₄), filtered and concentrated. The crude product was purified by flash chromatography (Silica, 0-50% of EtOAc in isohexane) to afford the product (1.1 g, 56% yield) as a thick colourless oil.

[M-boc+H]⁺=332.3/334.3

¹H NMR (500 MHz, DMSO-d6) δ 1.40 (9H, s), 1.41-1.44 (2H, m), 1.76-1.87 (2H, m), 2.98-3.11 (2H, m), 3.52-3.59 (1H, m), 3.59-3.68 (2H, m), 4.52 (2H, s), 7.15 (1H, d, J=8.2, 1.9 Hz), 7.33 (1H, d, J=9.8, 1.9 Hz), 7.68 (1H, t, J=7.8 Hz).

19F NMR (471 MHz, DMSO) δ −108.62.

### 4-((4-Bromo-3-fluorobenzyl)oxy)-1-methylpiperidine

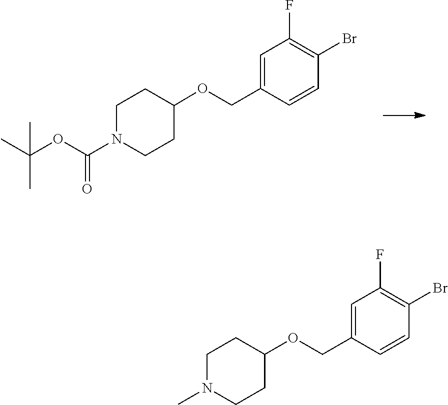 [SMILES: CC(C)(C)OC(=O)N1CCC(OCc2ccc(Br)c(F)c2)CC1 ; CN1CCC(OCc2ccc(Br)c(F)c2)CC1]

Following General Method 10, tert-butyl 4-((4-bromo-3-fluorobenzyl)oxy)piperidine-1-carboxylate (1.10 g, 2.83 mmol) was reacted at 90° C. for 3 h. The product was isolated as a colourless gum following elution through an SCX (696 mg, 79% yield).

[M+H]⁺=302.2/304.2

¹H NMR (500 MHz, DMSO-d6) δ 1.45-1.55 (2H, m), 1.81-1.88 (2H, m), 1.95-2.05 (2H, m), 2.13 (3H, s), 2.55-2.62 (2H, m), 3.34-3.41 (1H, m), 4.49 (2H, s), 7.14 (1H, d, J=8.2, 1.9 Hz), 7.31 (1H, d, J=9.8, 1.9 Hz), 7.67 (1H, t, J=7.8 Hz).

¹⁹F NMR (471 MHz, DMSO) δ −108.68.

### 2-Fluoro-4-(((1-methylpiperidin-4-yl)oxy)methyl)benzonitrile

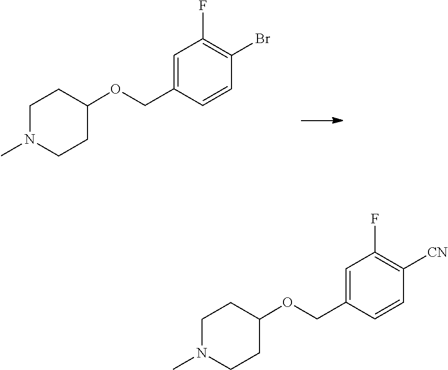 [SMILES: CN1CCC(OCc2ccc(Br)c(F)c2)CC1 ; CN1CCC(OCc2ccc(C#N)c(F)c2)CC1]

Using General Method 2, 4-((4-bromo-3-fluorobenzyl)oxy)-1-methylpiperidine (350 mg, 1.16 mmol) was reacted for 16 h. The crude product was purified by flash chromatography (Silica, 0-10% (0.7M NH₃ in MeOH) in DCM) to afford the product (230 mg, 79% yield) as a colourless oil.

[M+H]⁺=249.4

¹H NMR (500 MHz, DMSO-d6) δ 1.47-1.57 (2H, m), 1.82-1.90 (2H, m), 1.96-2.07 (2H, m), 2.15 (3H, s), 2.56-2.64 (2H, m), 3.35-3.45 (1H, m), 4.61 (2H, s), 7.37 (1H, dd, J=8.0, 1.4 Hz), 7.45 (1H, dd, J=10.5, 1.3 Hz), 7.88-7.93 (1H, m).

### (2-Fluoro-4-(((1-methylpiperidin-4-yl)oxy)methyl)phenyl)methanamine

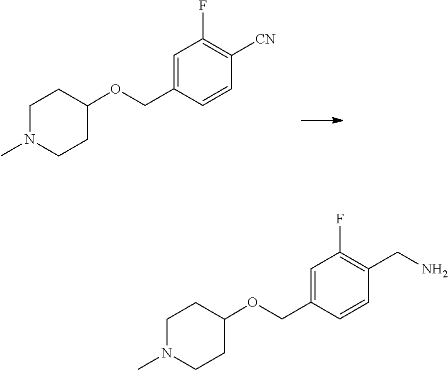 [SMILES: CN1CCC(OCc2ccc(C#N)c(F)c2)CC1 ; CN1CCC(OCc2ccc(CN)c(F)c2)CC1]

The nitrile, 2-fluoro-4-(((1-methylpiperidin-4-yl)oxy)methyl)benzonitrile (220 mg, 0.89 mmol) was reduced following General Method 3b, at rt for 3 h. The product was isolated (206 mg, 88% yield) as a colourless solid and used without further purification.

[M+H]⁺=253.4

¹H NMR (500 MHz, DMSO-d6) δ 1.44-1.55 (2H, m), 1.78 (2H, s), 1.78-1.88 (2H, m), 1.93-2.03 (2H, m), 2.13 (3H, s), 2.54-2.62 (2H, m), 3.33-3.39 (1H, m), 3.72 (2H, s), 4.47 (2H, s), 7.05 (1H, dd, J=11.1, 1.6 Hz), 7.09-7.14 (1H, m), 7.44 (1H, t, J=7.9 Hz).

### Intermediate 7

### (6-((1-Methylpiperidin-4-yl)methoxy)pyridin-3-yl)methanamine

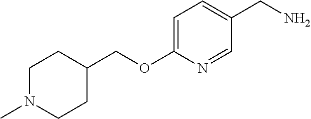 [SMILES: CN1CCC(COc2ccc(CN)cn2)CC1]

### 6-((1-Methylpiperidin-4-yl)methoxy)nicotinonitrile

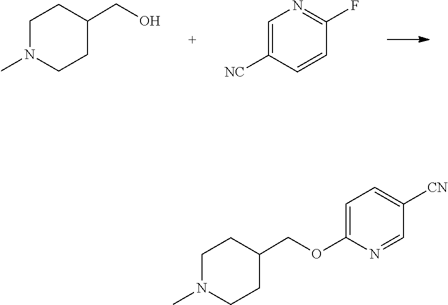

Using General Method 1a, (1-methylpiperidin-4-yl)methanol (300 mg, 2.32 mmol) was reacted with 6-fluoronicotinonitrile (284 mg, 2.32 mmol) for 20 h. The crude reaction mixture was passed directly through an SCX and washed with MeOH. The required product was eluted with 7M NH₃ in MeOH. The resultant mixture was concentrated and the crude product was purified by flash chromatography (Silica, 0-10% (0.7M NH₃ in MeOH) in DCM) to afford the product (250 mg, 42% yield) as a yellow solid.

[M+H]⁺=232.3

¹H NMR (500 MHz, DMSO-d6) δ 1.18-1.36 (2H, m), 1.63-1.73 (3H, m), 1.79-1.91 (2H, m), 2.15 (3H, s), 2.72-2.82 (2H, m), 4.19 (2H, d, J=6.2 Hz), 7.00 (1H, dd, J=8.7, 0.8 Hz), 8.14 (1H, dd, J=8.7, 2.4 Hz), 8.68 (1H, dd, J=2.4, 0.8 Hz).

### (6-((1-Methylpiperidin-4-yl)methoxy)pyridin-3-yl)methanamine

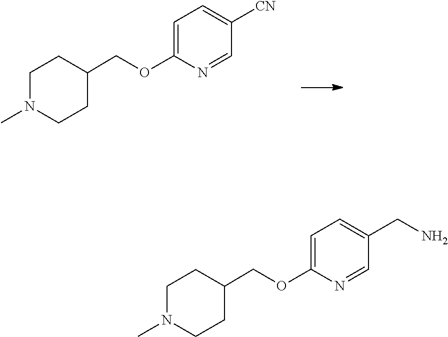 [SMILES: CN1CCC(COc2ccc(C#N)cn2)CC1 ; CN1CCC(COc2ccc(CN)cn2)CC1]

Reduction of nitrile 6-((1-methylpiperidin-4-yl)methoxy)nicotinonitrile (100 mg, 0.43 mmol) was carried out using General Method 3a, using a Raney Ni cartridge for 2 h. The product was isolated (78 mg, 74% yield) as a white solid.

[M+H]⁺=236.4

¹H NMR (500 MHz, DMSO-d6) δ 1.16-1.34 (2H, m), 1.61-1.74 (3H, m), 1.78-1.87 (2H, m), 2.14 (3H, s), 2.70-2.81 (2H, m), 3.64 (2H, s), 4.07 (2H, d, J=6.2 Hz), 6.74 (1H, d, J=8.4 Hz), 7.66 (1H, dd, J=8.5, 2.5 Hz), 8.03 (1H, d, J=2.4 Hz) (NH₂ not observed).

### Intermediate 8

### 5-(Aminomethyl)-N-((1-methylpiperidin-4-yl)methyl)pyridin-2-amine

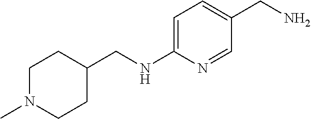 [SMILES: CN1CCC(CNc2ccc(CN)cn2)CC1]

### 6-(((1-Methylpiperidin-4-yl)methyl)amino)nicotinonitrile

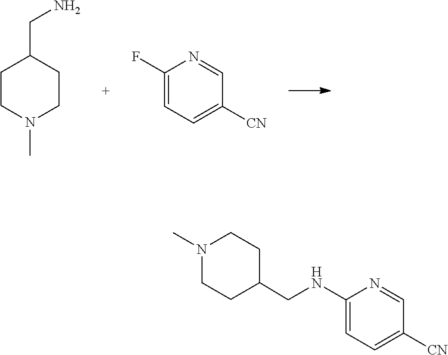

Following General Method 1d, using DIPEA (0.30 mL, 1.7 mmol) as base, 6-fluoronicotinonitrile (100 mg, 0.82 mmol) was reacted with (1-methylpiperidin-4-yl)methanamine (120 mg, 0.94 mmol) 80° C. for 30 minmin. The mixture was cooled to rt and concentrated. The crude product was purified by flash chromatography (Silica, 0-20% (0.7M NH₃ in MeOH) in EtOAc) to afford the product (154 mg, 78% yield) as a white solid.

[M+H]⁺=231.3

¹H NMR (500 MHz, DMSO-d 6) 1.10-1.24 (2H, m), 1.43-1.52 (1H, m), 1.60-1.69 (2H, m), 1.74-1.83 (2H, m), 2.12 (3H, s), 2.68-2.78 (2H, m), 3.12-3.25 (2H, m), 6.55 (1H, dd, J=8.9, 0.8 Hz), 7.57-7.70 (2H, m), 8.37 (1H, dd, J=2.3, 0.7 Hz).

### 5-(Aminomethyl)-N-((1-methylpiperidin-4-yl)methyl)pyridin-2-amine

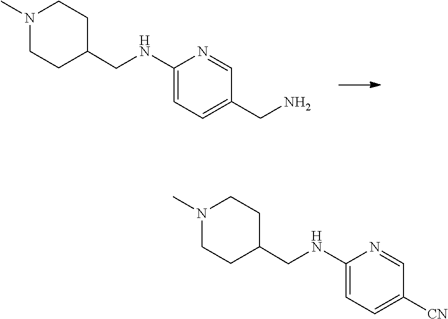 [SMILES: CN1CCC(CNc2ccc(CN)cn2)CC1 ; CN1CCC(CNc2ccc(C#N)cn2)CC1]

Following General Method 3a, 6-(((1-methylpiperidin-4-yl)methyl)amino)nicotinonitrile (100 mg, 0.43 mmol) was reduced using Raney Ni over 2 h. The mixture was concentrated to afford the product (77 mg, 72% yield) as a colourless oil.

[M+H]⁺=235.3

¹H NMR (500 MHz, DMSO-d 6) 1.09-1.20 (2H, m), 1.41-1.52 (1H, m), 1.61-1.69 (2H, m), 1.73-1.81 (2H, m), 2.12 (3H, s), 2.68-2.78 (2H, m), 3.05-3.12 (2H, m), 3.50 (2H, s), 6.32 (1H, t, J=5.8 Hz), 6.41 (1H, d, J=8.5 Hz), 7.32 (1H, dd, J=8.5, 2.4 Hz), 7.84 (1H, d, J=2.3 Hz), two exchangeable protons were not observed.

### Intermediate 9

### (2-((1-Methylpiperidin-4-yl)methoxy)pyridin-4-yl)methanamine

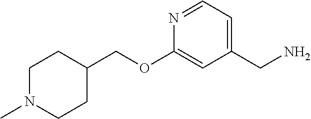 [SMILES: CN1CCC(COc2cc(CN)ccn2)CC1]

### 2-((1-Methylpiperidin-4-yl)methoxy)isonicotinonitrile

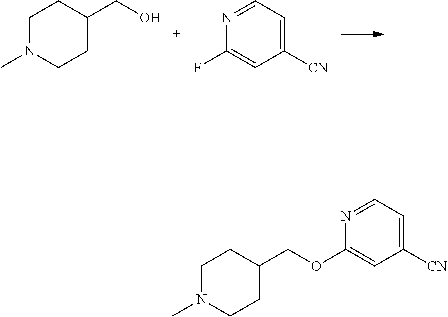

Following General Method 1a, using KO^{t}Bu (919 mg, 8.19 mmol) as base, (1-methylpiperidin-4-yl)methanol (529 mg, 4.10 mmol) was reacted with 2-fluoroisonicotinonitrile (500 mg, 4.10 mmol) for 18 h. The crude product was purified by flash chromatography (Silica, 0-10% (0.7M NH₃ in MeOH) DCM) to afford the product (451 mg, 45% yield) as a colourless oil.

[M+H]⁺=232.1

¹H NMR (500 MHz, DMSO-d6) δ 1.21-1.35 (2H, m), 1.64-1.74 (3H, m), 1.79-1.89 (2H, m), 2.15 (3H, s), 2.72-2.80 (2H, m), 4.15 (2H, d, J=6.2 Hz), 7.35-7.38 (1H, m), 7.39 (1H, dd, J=5.2, 1.3 Hz), 8.39 (1H, dd, J=5.2, 0.9 Hz).

### (2-((1-Methylpiperidin-4-yl)methoxy)pyridin-4-yl)methanamine

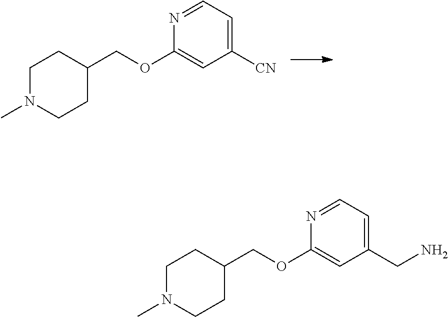 [SMILES: CN1CCC(COc2cc(C#N)ccn2)CC1 ; CN1CCC(COc2cc(CN)ccn2)CC1]

The nitrile, 2-((1-methylpiperidin-4-yl)methoxy)isonicotinonitrile (200 mg, 0.865 mmol) was reduced according to General Method 3a using Raney Ni for 2 h. The solvent was removed in vacuo to afford the product (205 mg, 97% yield) as a colourless solid.

[M+H]⁺=236.1

¹H NMR (500 MHz, DMSO-d6) δ 1.18-1.35 (2H, m), 1.68-1.72 (3H, m), 1.78-1.91 (2H, m), 2.15 (3H, s), 2.59 (2H, s), 2.70-2.83 (2H, m), 3.70 (2H, s), 4.08 (2H, d, J=6.1 Hz), 6.73-6.80 (1H, m), 6.91 (1H, dd, J=5.2, 1.4 Hz), 8.02 (1H, d, J=5.2 Hz).

### Intermediate 10

### 4-(Aminomethyl)-N-[(1-methyl-4-piperidyl)methyl]pyridin-2-amine

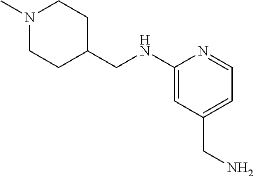 [SMILES: CN1CCC(CNc2cc(CN)ccn2)CC1]

### 2-[(1-Methyl-4-piperidyl)methylamino]pyridine-4-carbonitrile

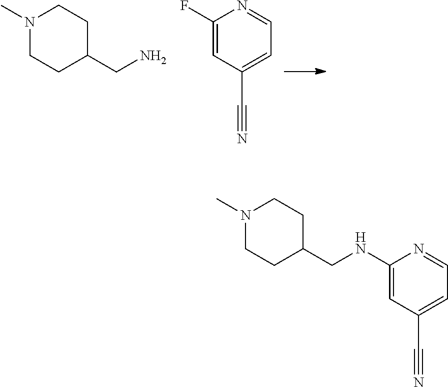

Following General Method 1b, (1-methylpiperidin-4-yl)methanamine (231 mg, 1.80 mmol) was reacted with 2-fluoropyridine-4-carbonitrile (200 mg, 1.64 mmol) at 60° C. for 48 h. Following aqueous work up, the crude product was purified by flash chromatography (Amino-D, 0-100% EtOAc in Pet. Ether) to afford the product (190 mg, 44% yield) as yellow oil that solidified on standing.

[M+H]⁺=231.0

¹H NMR (CDCl₃, 400 MHz) δ 1.29-1.45 (2H, m), 1.52-1.63 (1H, m), 1.74-1.81 (3H, m should be 2H, partially obscured by water), 1.92 (2H, td, J=11.8, 2.6 Hz), 2.27 (3H, s), 2.87 (2H, dt, J=12.1, 3.8 Hz), 3.19 (2H, dd, J=6.8, 6.0 Hz), 4.88 (1H, s), 6.55 (1H, t, J=1.1 Hz), 6.72 (1H, dd, J=5.1, 1.3 Hz), 8.18 (1H, dd, J=5.1, 0.9 Hz).

### 4-(Aminomethyl)-N-[(1-methyl-4-piperidyl)methyl]pyridin-2-amine

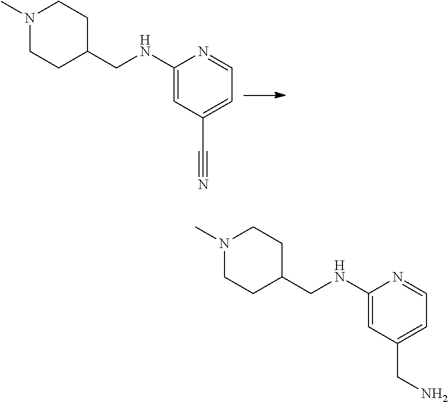

The nitrile, 2-[(1-methyl-4-piperidyl)methylamino]pyridine-4-carbonitrile (120 mg, 0.46 mmol) was reduced following General Method 3e, in the presence of palladium hydroxide on carbon (70 mg, 0.09 mmol) and 10% Pd/C (98 mg, 0.09 mmol) for 7 h. The mixture was filtered through Celite® and concentrated to afford the product (110 mg, 72% yield) as transparent semi-solid.

[M+H]⁺=235.1

### Intermediate 11

### Tert-butyl 4-(((4-(aminomethyl)pyridin-2-yl)oxy)methyl)piperidine-1-carboxylate

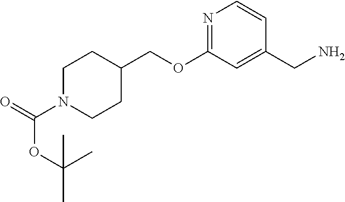 [SMILES: CC(C)(C)OC(=O)N1CCC(COc2cc(CN)ccn2)CC1]

### Tert-butyl 4-(((4-cyanopyridin-2-yl)oxy)methyl)piperidine-1-carboxylate

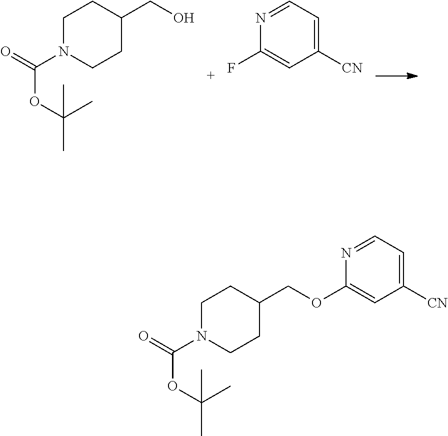 [SMILES: CC(C)(C)OC(=O)N1CCC(CO)CC1 ; N#Cc1ccnc(F)c1 ; CC(C)(C)OC(=O)N1CCC(COc2cc(C#N)ccn2)CC1]

Following General Method 1b, tert-butyl 4-(hydroxymethyl)piperidine-1-carboxylate (353 mg, 1.64 mmol) was reacted with 2-fluoroisonicotinonitrile (200 mg, 1.64 mmol) for 18 h. The reaction mixture was cooled to rt and diluted with water (10 mL). The crude product was extracted into DCM (2×25 mL), dried (MgSO₄), filtered and concentrated. The crude product was purified by flash chromatography (Silica, 5-100% EtOAc in Pet. Ether) to afford the product (500 mg, 96% yield) as a pale yellow oil.

[M-boc+H]⁺=218.1

¹H NMR (400 MHz, CDCl₃) δ 1.21-1.32 (2H, m), 1.47 (9H, s), 1.80 (2H, d, J=12.9 Hz), 1.92-2.02 (1H, m), 2.75 (2H, t, J=11.8 Hz), 4.09-4.20 (4H, m), 6.99 (1H, d, J=0.9 Hz), 7.07 (1H, dd, J=5.1, 1.3 Hz), 8.28 (1H, d, J=5.0 Hz) ppm.

### Tert-butyl 4-(((4-(aminomethyl)pyridin-2-yl)oxy)methyl)piperidine-1-carboxylate

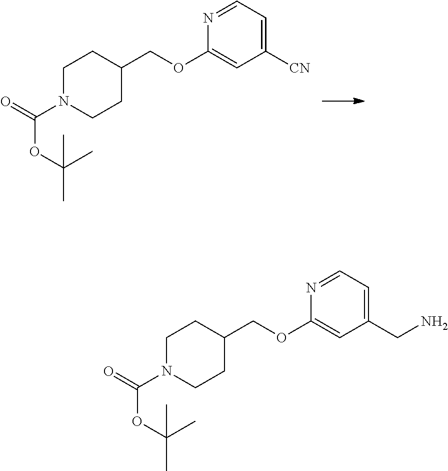 [SMILES: CC(C)(C)OC(=O)N1CCC(COc2cc(C#N)ccn2)CC1 ; CC(C)(C)OC(=O)N1CCC(COc2cc(CN)ccn2)CC1]

Following General Method 3a, the nitrile, tert-butyl 4-(((4-cyanopyridin-2-yl)oxy)methyl)piperidine-1-carboxylate (500 mg, 1.58 mmol) was reduced using Raney Ni. The solvent was removed in vacuo to afford the product (497 mg, 98% yield) as a colourless oil.

[M+H]⁺=322.1

¹H NMR (CDCl₃, 400 MHz) δ 1.25 (2H, qd, J=12.4, 4.4 Hz), 1.46 (9H, s), 1.73-1.83 (2H, m), 1.89-2.00 (1H, m), 2.33 (2H, br s), 2.73 (2H, t, J=12.8 Hz), 3.86 (2H, s), 4.04-4.19 (4H, m), 6.65-6.75 (1H, m), 6.77-6.88 (1H, m), 8.07 (1H, dd, J=5.3, 0.7 Hz) ppm

### Intermediate 12

### 1-(5-(((4-(Aminomethyl)pyridin-2-yl)oxy)methyl)-2-azabicyclo[2.2.1]heptan-2-yl)ethan-1-one

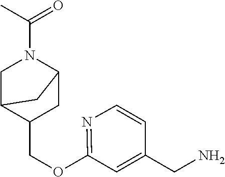 [SMILES: CC(=O)N1CC2CC1CC2COc1cc(CN)ccn1]

### Tert-butyl 5-(((4-cyanopyridin-2-yl)oxy)methyl)-2-azabicyclo[2.2.1]heptane-2-carboxylate

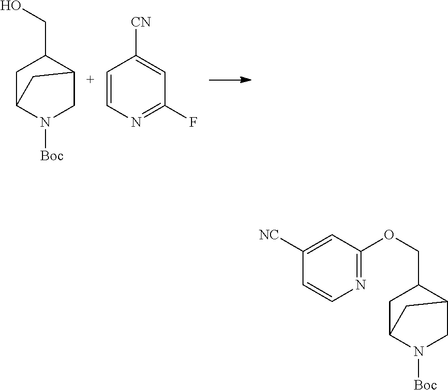

Following General Method 1b, tert-butyl 5-(hydroxymethyl)-2-azabicyclo[2.2.1]heptane-2-carboxylate (500 mg, 2.20 mmol) was reacted with 2-fluoroisonicotinonitrile (269 mg, 2.20 mmol) for 72 h. The reaction mixture was filtered and purified by flash chromatography (Silica, 0-60% EtOAc in isohexane) to afford the product (1.01 g, 65% yield) as a colourless solid.

[M+Na]⁺=352.2

¹H NMR (500 MHz, DMSO-d6) 1.10-1.19 (1H, m), 1.39 (9H, s), 1.51-1.61 (1H, m), 1.63-1.73 (1H, m), 1.80-1.91 (1H, m), 2.45-2.49 (1H, m), 2.54-2.58 (1H, m), 3.01-3.11 (1H, m), 3.20-3.25 (1H, m), 4.02 (1H, d, J=14.2 Hz), 4.12-4.20 (1H, m), 4.32-4.42 (1H, m), 7.36-7.38 (1H, m), 7.41 (1H, dd, J=5.2, 1.4 Hz), 8.40 (1H, dd, J=5.2, 0.8 Hz).

### 2-((2-Azabicyclo[2.2.1]heptan-5-yl)methoxy)isonicotinonitrile

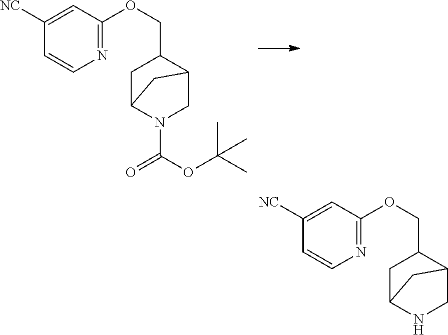

Boc deprotection of tert-butyl 5-(((4-cyanopyridin-2-yl)oxy)methyl)-2-azabicyclo[2.2.1]heptane-2-carboxylate (337 mg, 1.02 mmol) was carried out following General Method 7b. After elution through an SCX, the product was isolated (233 mg, 94% yield) and used without further purification.

[M+H]⁺=230.1

¹H NMR (500 MHz, DMSO-d6) 0.95-1.03 (1H, m), 1.41-1.51 (2H, m), 1.72-1.79 (1H, m), 2.27-2.40 (2H, m), 2.57-2.62 (1H, m), 2.79 (1H, d, J=9.9 Hz), 3.24-3.27 (1H, m), 4.28 (1H, dd, J=10.7, 9.1 Hz), 4.40 (1H, dd, J=10.7, 6.6 Hz), 7.37 (1H, s), 7.39 (1H, dd, J=5.3, 1.4 Hz), 8.40 (1H, d, J=5.2 Hz), NH not observed

### 2-((2-Acetyl-2-azabicyclo[2.2.1]heptan-5-yl)methoxy)isonicotinonitrile

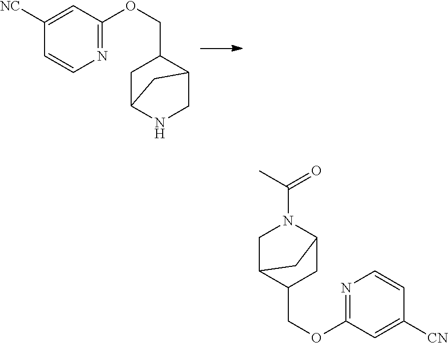 [SMILES: N#Cc1ccnc(OCC2CC3CC2CN3)c1 ; CC(=O)N1CC2CC1CC2COc1cc(C#N)ccn1]

A solution of 2-((2-azabicyclo[2.2.1]heptan-5-yl)methoxy)isonicotinonitrile (233 mg, 1.02 mmol) in DCM (5 mL) was treated with DIPEA (400 μL, 2.30 mmol) and acetic anhydride (100 μL, 1.06 mmol) then stirred at rt for 18 h. The mixture was treated with 1M HCl (20 mL) and the layers separated. The aqueous was extracted with DCM (2×5 mL). The combined organics were dried (Na₂SO₄), filtered and concentrated to afford the product (280 mg, 99% yield) as a yellow gum.

[M+H]⁺=272.1

### 1-(5-(((4-(Aminomethyl)pyridin-2-yl)oxy)methyl)-2-azabicyclo[2.2.1]heptan-2-yl)ethan-1-one

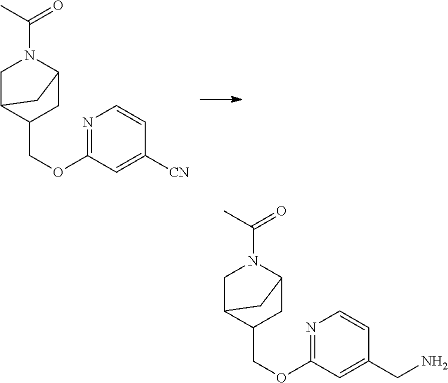 [SMILES: CC(=O)N1CC2CC1CC2COc1cc(C#N)ccn1 ; CC(=O)N1CC2CC1CC2COc1cc(CN)ccn1]

Reduction of the nitrile, 2-((2-acetyl-2-azabicyclo[2.2.1]heptan-5-yl)methoxy)isonicotinonitrile (280 mg, 1.03 mmol) was performed following General Method 3a for 3 h using Raney Ni. The resultant solution was concentrated to give the product (250 mg, 86% yield) as a colourless solid.

[M+H]⁺=276.2

### Intermediate 13

### (1-(2-(1-Methylpiperidin-4-yl)ethyl)-1H-pyrazol-4-yl)methanamine

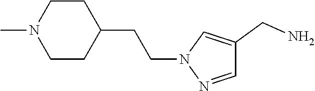 [SMILES: CN1CCC(CCn2cc(CN)cn2)CC1]

### Tert-butyl 4-(2-(4-cyano-1H-pyrazol-1-yl)ethyl)piperidine-1-carboxylate

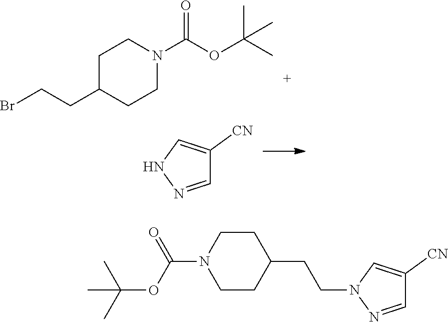

Following General Method 5b, tert-butyl 4-(2-bromoethyl)piperidine-1-carboxylate (800 mg, 2.74 mmol) was reacted with 1H-pyrazole-4-carbonitrile (255 mg, 2.74 mmol) and K₂CO₃ (720 mg, 5.21 mmol) in NMP (4 mL). The crude product was purified by flash chromatography (Silica, 0-100% EtOAc in iso-hexane) to afford the product (740 mg, 80% yield) as a colourless gum.

[M+H]⁺=248.2

### 1-(2-(1-Methylpiperidin-4-yl)ethyl)-1H-pyrazole-4-carbonitrile

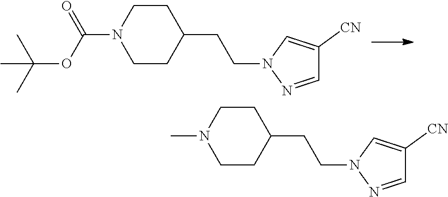

Tert-butyl 4-(2-(4-cyano-1H-pyrazol-1-yl)ethyl)piperidine-1-carboxylate (0.85 g, 2.79 mmol) was reacted using General Method 10 min at 90° C. for 2 h. The crude product was purified by flash chromatography (Silica, 0-10% MeOH in DCM) to afford the product (254 mg, 40% yield) as a colourless gum.

¹H NMR (500 MHz, DMSO-d6) δ 1.04-1.20 (3H, m), 1.57-1.65 (2H, m), 1.68-1.74 (2H, m), 1.74-1.81 (2H, m), 2.13 (3H, s), 2.69-2.74 (2H, m), 4.20 (2H, t, J=7.3 Hz), 8.05 (1H, s), 8.59 (1H, s)

### (1-(2-(1-Methylpiperidin-4-yl)ethyl)-1H-pyrazol-4-yl)methanamine

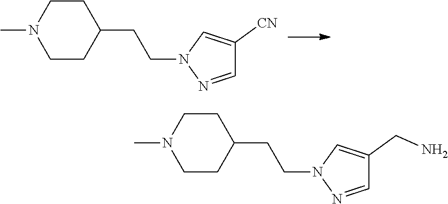

The nitrile 1-(2-(1-methylpiperidin-4-yl)ethyl)-1H-pyrazole-4-carbonitrile (154 mg, 0.71 mmol) was reduced according to General Method 3b and reacted for 18 h. The product (135 mg, 80%) was isolated as a colourless gum and used without further purification.

¹H NMR (500 MHz, DMSO-d6) δ 1.04-1.19 (3H, m), 1.45-1.69 (6H, m), 1.69-1.79 (2H, m), 2.11 (3H, s), 2.66-2.73 (2H, m), 3.55 (2H, s), 4.05 (2H, t, J=7.3 Hz), 7.30 (1H, s), 7.51-7.55 (1H, m).

### Intermediate 14

### (2-((5,6,7,8-Tetrahydroimidazo[1,2-a]pyridin-7-yl)methoxy)pyridin-4-yl)methanamine

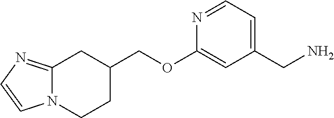 [SMILES: NCc1ccnc(OCC2CCn3ccnc3C2)c1]

### 2-((5,6,7,8-Tetrahydroimidazo[1,2-a]pyridin-7-yl)methoxy)isonicotinonitrile

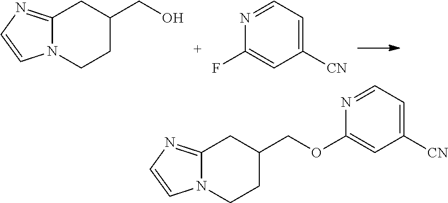

Following General Method 1b, (5,6,7,8-tetrahydroimidazo[1,2-a]pyridin-7-yl)methanol (200 mg, 1.31 mmol) was reacted with 2-fluoroisonicotinonitrile (321 mg, 2.63 mmol) for 18 h. Following aqueous work up, the crude product was purified by flash chromatography (Silica, 0-20% MeOH in DCM) to afford the product (214 mg, 61% yield) as an orange oil.

[M+H]⁺=255.0

### (2-((5,6,7,8-Tetrahydroimidazo[1,2-a]pyridin-7-yl)methoxy)pyridin-4-yl)methanamine

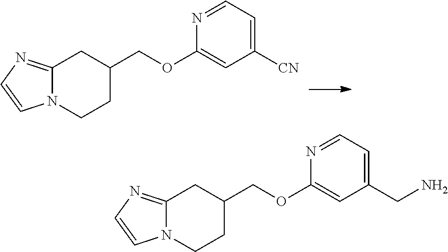 [SMILES: N#Cc1ccnc(OCC2CCn3ccnc3C2)c1 ; NCc1ccnc(OCC2CCn3ccnc3C2)c1]

The nitrile, 2-((5,6,7,8-tetrahydroimidazo[1,2-a]pyridin-7-yl)methoxy)isonicotinonitrile (214 mg, 0.84 mmol) was reduced according to General Method 3a using Raney Ni over 3 h. The solvent was removed in vacuo to afford the product (216 mg, 99% yield) as an orange oil.

[M+Na]⁺=259.0

### Intermediate 15

### (6-((5,6,7,8-Tetrahydroimidazo[1,2-a]pyridin-7-yl)methoxy)pyridin-3-yl)methanamine

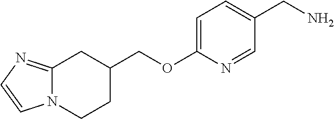 [SMILES: NCc1ccc(OCC2CCn3ccnc3C2)nc1]

### 6-((5,6,7,8-Tetrahydroimidazo[1,2-a]pyridin-7-yl)methoxy)nicotinonitrile

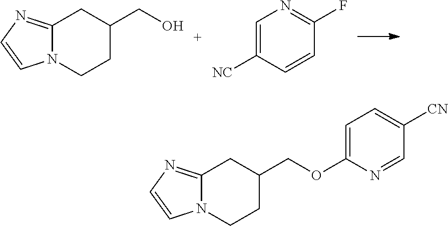 [SMILES: OCC1CCn2ccnc2C1 ; N#Cc1ccc(F)nc1 ; N#Cc1ccc(OCC2CCn3ccnc3C2)nc1]

Following General Method 1b, (5,6,7,8-tetrahydroimidazo[1,2-a]pyridin-7-yl)methanol (890 mg, 5.85 mmol) was reacted with 6-fluoronicotinonitrile (714 mg, 5.85 mmol) for 5 h. After elution through an SCX, the crude product was purified by flash chromatography (Silica, 0-10% (0.7 NH₃ in MeOH) in DCM) to afford the product (723 mg, 44% yield) as a pale brown solid.

[M+H]⁺=255.3

¹H NMR (500 MHz, DMSO-d6) δ 1.68-1.80 (1H, m), 2.10-2.18 (1H, m), 2.35-2.45 (1H, m), 2.51-2.55 (1H, m), 2.94 (1H, ddd, J=16.2, 5.0, 1.5 Hz), 3.84-3.94 (1H, m), 4.04-4.13 (1H, m), 4.37 (2H, d, J=6.6 Hz), 6.81 (1H, d, J=1.2 Hz), 7.00 (1H, d, J=1.2 Hz), 7.06 (1H, dd, J=8.7, 0.8 Hz), 8.18 (1H, dd, J=8.7, 2.4 Hz), 8.71 (1H, dd, J=2.4, 0.8 Hz)

### (6-((5,6,7,8-Tetrahydroimidazo[1,2-a]pyridin-7-yl)methoxy)pyridin-3-yl)methanamine

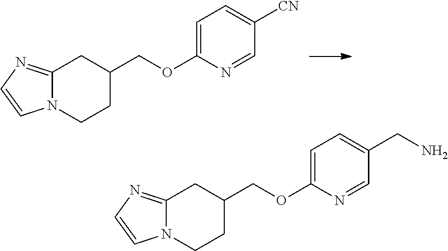 [SMILES: N#Cc1ccc(OCC2CCn3ccnc3C2)nc1 ; NCc1ccc(OCC2CCn3ccnc3C2)nc1]

Reduction of 6-((5,6,7,8-tetrahydroimidazo[1,2-a]pyridin-7-yl)methoxy)nicotinonitrile (200 mg, 0.79 mmol) was carried out using General Method 3a, using Raney Ni over 2 h. The reaction mixture was concentrated to afford the product (190 mg, 87% yield) as a clear yellow oil.

[M+H]⁺=259.0

¹H NMR (500 MHz, DMSO-d6) δ 1.65-1.78 (1H, m), 2.09-2.17 (1H, m), 2.32-2.43 (1H, m), 2.45-2.53 (1H, m), 2.93 (1H, ddd, J=16.2, 5.1, 1.6 Hz), 3.65 (2H, s), 3.84-3.94 (1H, m), 4.04-4.13 (1H, m), 4.22-4.27 (2H, m), 6.78-6.83 (2H, m), 6.99 (1H, d, J=1.3 Hz), 7.69 (1H, dd, J=8.5, 2.5 Hz), 8.06 (1H, d, J=2.5 Hz), (NH₂ not seen).

### Intermediate 16

### 5-Bromo-1-((2-(trimethylsilyl)ethoxy)methyl)-1H-indole

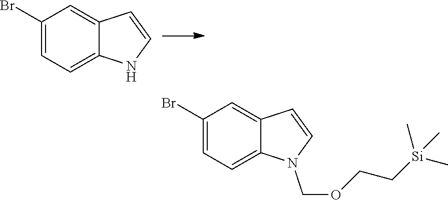 [SMILES: Brc1ccc2[nH]ccc2c1 ; C[Si](C)(C)CCOCn1ccc2cc(Br)ccc21]

Following General Method 5a 5-bromo-1H-indole (1.0 g, 5.1 mmol) was reacted with SEM-CI (5.7 mmol) at rt for 1 h. Sat. NH₄Cl aq. (30 mL) was added and extracted with TBME (30 mL). The organics were washed with brine/water (1:1, 30 mL) and brine (2×30 mL) before being dried (MgSO₄), filtered and concentrated. The crude product was purified by flash chromatography (silica, 0-10% TBME/Hexane) to afford the product (1.13 g, 64% yield) as a colourless gum. ¹H NMR (500 MHz, DMSO-d6) −0.10 (9H, s), 0.77-0.83 (2H, m), 3.40-3.46 (2H, m), 5.55 (2H, s), 6.48 (1H, dd, J=3.2, 0.8 Hz), 7.29 (1H, dd, J=8.7, 2.0 Hz), 7.52-7.55 (2H, m), 7.76 (1H, d, J=1.9 Hz).

### Intermediate 17

### 5-Bromo-3-chloro-1-((2-(trimethylsilyl)ethoxy)methyl)-1H-indole

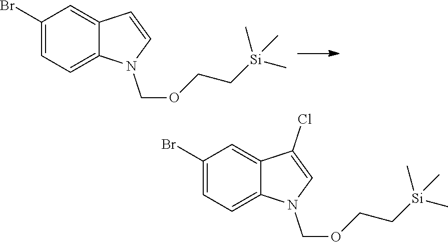 [SMILES: C[Si](C)(C)CCOCn1ccc2cc(Br)ccc21 ; C[Si](C)(C)CCOCn1cc(Cl)c2cc(Br)ccc21]

Following General Method 6b, 5-bromo-1-((2-(trimethylsilyl)ethoxy)methyl)-1H-indole (1.13 g, 3.46 mmol) in DCM (25 mL) was reacted with NCS (500 mg, 3.74 mmol) at rt for 18 h. After the aqueous work up, the crude was purified by flash chromatography (silica, 0-5% EtOAc/isohexane) to afford the product (830 mg, 60% yield) as a yellow gum.

¹H NMR (500 MHz, DMSO-d6) −0.10 (9H, s), 0.77-0.82 (2H, m), 3.42-3.47 (2H, m), 5.54 (2H, s), 7.41 (1H, dd, J=8.7, 2.0 Hz), 7.62 (1H, dd, J=8.7, 0.6 Hz), 7.66 (1H, dd, J=2.0, 0.5 Hz), 7.79 (1H, s).

### Intermediate 18

### 5-Bromo-3-chloro-1-((2-(trimethylsilyl)ethoxy)methyl)-1H-pyrrolo[2,3-b]pyridine

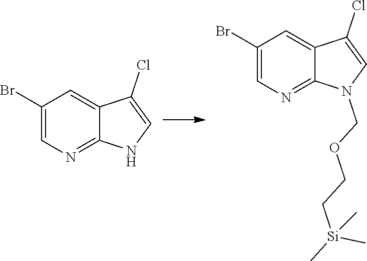 [SMILES: Clc1c[nH]c2ncc(Br)cc12 ; C[Si](C)(C)CCOCn1cc(Cl)c2cc(Br)cnc21]

Following General Method 5a, 5-bromo-3-chloro-1H-pyrrolo[2,3-b]pyridine (480 mg, 2.07 mmol) was reacted (2-(chloromethoxy)ethyl)trimethylsilane (0.4 mL, 2.28 mmol) for 2 h. The reaction was quenched with water (2 mL) and diluted with EtOAc (40 mL). The organic layer was washed with water (20 mL), 1M HCl (aq) (20 mL), 1:1 water/brine (20 mL) and brine (20 mL), dried (MgSO₄), filtered and concentrated. The crude product was purified by flash chromatography (Silica, 0-100% EtOAc in iso-hexane) to afford the product (485 mg, 60% yield) as an orange oil.

[M+H]⁺=363.0

¹H NMR (500 MHz, DMSO-d6) δ −0.10 (9H, s), 0.81 (2H, t, J=7.9 Hz), 3.51 (2H, t, J=7.9 Hz), 5.60 (2H, s), 7.98-8.01 (1H, m), 8.20-8.24 (1H, m), 8.44-8.47 (1H, m)

### Intermediate 19

### Tert-butyl (6-bromoisoquinolin-1-yl)carbamate

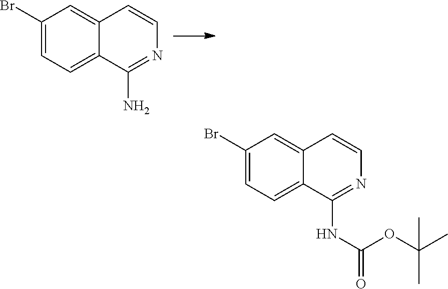 [SMILES: Nc1nccc2cc(Br)ccc12 ; CC(C)(C)OC(=O)Nc1nccc2cc(Br)ccc12]

A solution of 6-bromoisoquinolin-1-amine (0.50 g, 2.20 mmol) in tBuOH (10 mL) at 40° C. was treated with Boc₂O (0.49 g, 2.20 mmol) and heated to 70° C. for 18 h. The reaction mixture was concentrated and the crude product was purified by flash chromatography (Silica, 0-100% EtOAc in isohexane) to afford the product (457 mg, 60% yield) as a colourless solid.

[M+H]⁺=322.9

¹H NMR (500 MHz, DMSO-d6) δ 1.48 (9H, s), 7.64 (1H, d, J=5.7 Hz), 7.79 (1H, d, J=8.9 Hz), 8.00 (1H, d, J=9.0 Hz), 8.27 (1H, s), 8.31 (1H, d, J=5.7 Hz), 9.85 (1H, s).

### Intermediate 20

### Tert-butyl (6-bromoisoquinolin-1-yl)(methyl)carbamate

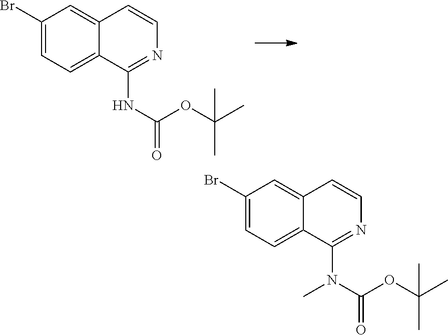

A mixture of tert-butyl (6-bromoisoquinolin-1-yl)carbamate (150 mg, 0.46 mmol) and methyl iodide (35 μL, 0.56 mmol) in THF (2 mL) was cooled in an ice/water bath. NaH (60% in mineral oil) (23 mg, 0.60 mmol) was added and the mixture was warmed to rt and stirred for 18 h. The reaction was quenched with MeOH (0.5 mL) and concentrated. The crude mixture was taken up into water (20 mL) and extracted into EtOAc (2×20 mL), the combined organic layers were dried (MgSO₄), filtered and concentrated. The crude product was purified by flash chromatography (Silica, 0-50% EtOAc in isohexane) to give the product (104 mg, 64% yield) as a cream solid.

[M+H]⁺=281.1/283.1

¹H NMR (500 MHz, DMSO-d6) δ 1.22 (9H, s), 3.29 (3H, s), 7.78 (1H, d, J=5.7 Hz), 7.81-7.88 (2H, m), 8.35 (1H, d, J=1.9 Hz), 8.42 (1H, d, J=5.7 Hz).

### Intermediate 21

### Tert-butyl (5-bromoisoquinolin-1-yl)carbamate

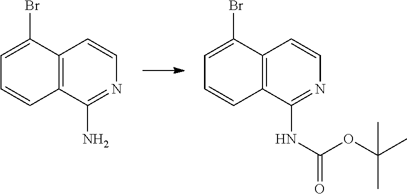 [SMILES: Nc1nccc2c(Br)cccc12 ; CC(C)(C)OC(=O)Nc1nccc2c(Br)cccc12]

A suspension of 5-bromoisoquinolin-1-amine (700 mg, 3.14 mmol) in tBuOH (6 mL) was treated with Boc₂O (1.5 g, 6.90 mmol) and heated to 70° C. for 18 h. The reaction mixture was concentrated, then taken up in MeOH (30 mL) and K₂CO₃ (860 mg, 6.22 mmol) was added and the reaction mixture was heated at 70° C. for 1 h. This was allowed to cool to rt, filtered and concentrated. The crude product was purified by flash chromatography (Silica, 100% DCM) to afford the product (700 mg, 52% yield) as a yellow solid.

[M-boc+H]⁺=323.0

### Intermediate 22

### Tert-butyl (6-bromoisoquinolin-3-yl)carbamate

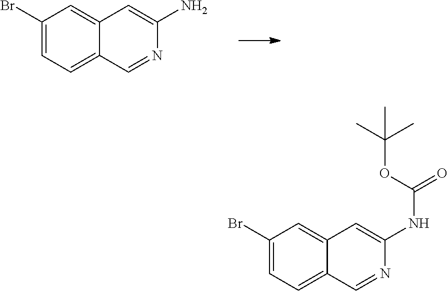 [SMILES: Nc1cc2cc(Br)ccc2cn1 ; CC(C)(C)OC(=O)Nc1cc2cc(Br)ccc2cn1]

A solution of 6-bromoisoquinolin-3-amine (1.0 g, 4.48 mmol) in tBuOH (10 mL) was treated with Boc₂O (1.47 g, 6.72 mmol) and heated to 70° C. for 18 h. The reaction mixture was concentrated and purified by flash chromatography (Silica, 5-100% THF in isohexane) to afford the product (825 mg, 54% yield) as a tan solid.

[M+H]⁺=323.0

¹H NMR (500 MHz, DMSO-d6) δ 1.51 (9H, s), 7.59 (1H, dd, J=8.7, 1.9 Hz), 7.97 (1H, d, J=8.7 Hz), 8.12-8.13 (1H, m), 8.17-8.18 (1H, m), 9.09-9.10 (1H, m), 9.96 (1H, s).

### Intermediate 23

### Methyl (6-bromoisoquinolin-1-yl)carbamate

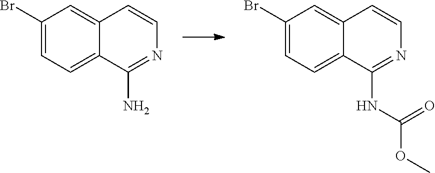 [SMILES: Nc1nccc2cc(Br)ccc12 ; COC(=O)Nc1nccc2cc(Br)ccc12]

Following General Method 13, 6-bromoisoquinolin-1-amine (1.50 g, 6.72 mmol) was protected. The crude was suspended in water (100 mL) and stirred for 30 min before being collected by filtration and dried in the vacuum oven overnight to give the product (1.12 g, 44% yield) as an off-white solid.

[M+H]⁺=281.1

¹H NMR (500 MHz, DMSO-d6) 3.70 (3H, s), 7.58-7.72 (1H, m), 7.79 (1H, d, J=9.0, 2.0 Hz), 8.04 (1H, d, J=9.1 Hz), 8.25-8.30 (1H, m), 8.33 (1H, d, J=5.8 Hz), 10.18 (1H, s)

### Intermediate 24

### Methyl (6-bromo-4-chloroisoquinolin-1-yl)carbamate

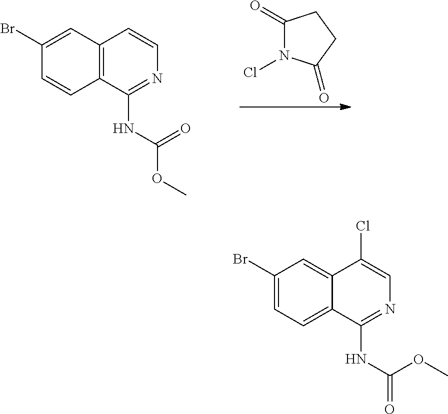 [SMILES: COC(=O)Nc1nccc2cc(Br)ccc12 ; O=C1CCC(=O)N1Cl ; COC(=O)Nc1ncc(Cl)c2cc(Br)ccc12]

Methyl N-(6-bromo-1-isoquinolyl)carbamate (100 mg, 0.36 mmol) was dissolved in chloroform (5 mL), NCS (52 mg, 0.39 mmol) was added and the reaction stirred at reflux for 18 h. To the reaction was added sat. NaHCO₃ (aq.) (30 mL) and it was washed with DCM (30 mL), dried (Na₂SO₄) and concentrated. The crude product was purified by flash chromatography (Silica, 0-80% EtOAc in Pet. Ether) to give the product (74 mg, 59% yield) as light beige solid.

[M+H]⁺=316.8/318.7

¹H NMR (CDCl₃, 400 MHz) δ 3.84 (3H, s), 7.36 (1H, s), 7.75 (1H, dd, J=9.0, 1.9 Hz), 7.93 (1H, d, J=9.0 Hz), 8.37 (2H, d, J=4.9 Hz)

### Intermediate 25

### Methyl (5-bromoisoquinolin-1-yl)carbamate

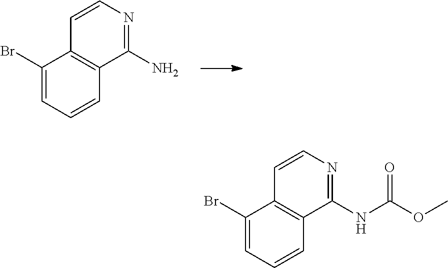 [SMILES: Nc1nccc2c(Br)cccc12 ; COC(=O)Nc1nccc2c(Br)cccc12]

Following General Method 13, 5-bromoisoquinolin-1-amine (1.12 g, 5.02 mmol) was protected. The product was dried under high vacuum to yield (838 mg, 56% yield) [M+H]⁺=281.1

### Intermediate 26

### 6-Chloro-N-(2,4-dimethoxybenzyl)-2,7-naphthyridin-1-amine

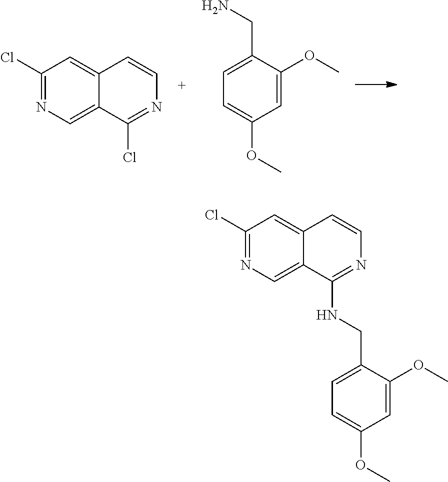

Following General Method 1c, 1,6-dichloro-2,7-naphthyridine (200 mg, 1.00 mmol) was protected in NMP (1 mL) at 100° C. for 1 h. This reaction mixture was taken up in water (20 mL) and MeOH (20 mL) and filtered to afford the product (212 mg, 45% yield) as an orange solid.

[M+H]⁺=330.1

¹H NMR (500 MHz, DMSO-d6) 3.73 (3H, s), 3.83 (3H, s), 4.63 (2H, d, J=5.4 Hz), 6.44 (1H, dd, J=8.4, 2.4 Hz), 6.58 (1H, d, J=2.3 Hz), 6.84 (1H, d, J=5.8 Hz), 7.12 (1H, d, J=8.3 Hz), 7.77 (1H, s), 8.05 (1H, d, J=5.8 Hz), 8.35 (1H, t, J=5.6 Hz), 9.50 (1H, s)

### Intermediate 27

### 5-Bromo-N-(2,4-dimethoxybenzyl)isoquinolin-1-amine

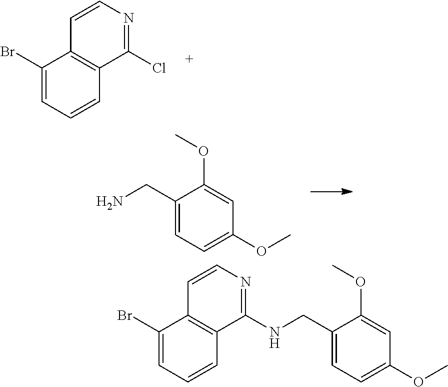

To a solution of 5-bromo-1-chloroisoquinoline (0.5 g, 2.06 mmol) in pyridine (3 mL), was added 2,4-dimethoxybenzylamine (0.69 g, 4.12 mmol). The reaction was heated at 150° C. in a CEM Microwave for 1 h. The mixture was diluted with DCM (20 mL) and water (20 mL). The aqueous layer was re-extracted with DCM (3×10 mL) and the combined organics were washed with brine (20 mL). The organic layer was dried (Na₂SO₄), filtered and concentrated to afford the crude product. Purification was performed by flash chromatography (Silica, 20-50% EtOAc in Pet ether) to afford the product (276 mg, 50% yield) as a pale yellow oil.

[M+H]⁺=373.0/375.0

¹H NMR (DMSO-d₆, 400 MHz) δ 3.71 (3H, d, J=2.6 Hz), 3.82 (3H, d, J=2.8 Hz), 4.62 (2H, d, J=5.4 Hz), 6.41 (1H, dd, J=8.5, 2.5 Hz), 6.56 (1H, d, J=2.6 Hz), 6.94-7.14 (2H, m), 7.42 (1H, t, J=8.0 Hz), 7.96 (3H, ddd, J=16.4, 7.1, 3.2 Hz), 8.38 (1H, d, J=8.2 Hz).

### Intermediate 28

### 6-Bromo-N-(2,4-dimethoxybenzyl)-4-fluoroisoquinolin-1-amine

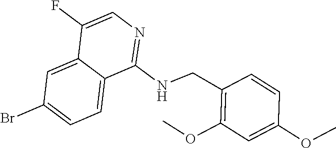 [SMILES: COc1ccc(CNc2ncc(F)c3cc(Br)ccc23)c(OC)c1]

### 6-Bromo-1-chloro-4-fluoroisoquinoline

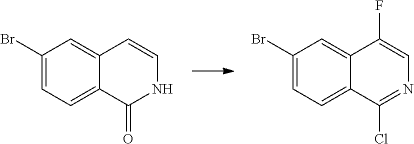 [SMILES: O=c1[nH]ccc2cc(Br)ccc12 ; Fc1cnc(Cl)c2ccc(Br)cc12]

A solution of 6-bromo-2H-isoquinolin-1-one (8.0 g, 35.7 mmol) and Selectfluor (15.2 g, 42.8 mmol) in MeCN (100 mL) and MeOH (100 mL) were heated at 50° C. for 1 h. The reaction mixture was evaporated and reacted using General Method 11, in 1,2-dichloroethane (200 mL) with benzyltriethylammonium chloride (820 mg, 3.6 mmol) and phosphorus oxychloride (50 mL). The reaction mixture was evaporated and the residue partitioned between DCM (500 mL) and water (500 mL). The organic layer was washed with water (300 mL), brine (300 mL), dried (MgSO₄) and evaporated. The crude was purified by flash chromatography (Silica, 5% EtOAc in Pet. Ether) to give the product as a cream solid (6.88 g, 74% yield).

[M+H]⁺=260.0

¹H NMR (500 MHz, CDCl₃) δ 8.27 (d, J=1.9 Hz, 1H), 8.21-8.16 (m, 2H), 7.84 (dd, J=9.1, 1.9 Hz, 1H). ¹⁹F NMR (471 MHz, CDCl₃) 5-139.8 (s).

### 6-Bromo-N-(2,4-dimethoxybenzyl)-4-fluoroisoquinolin-1-amine

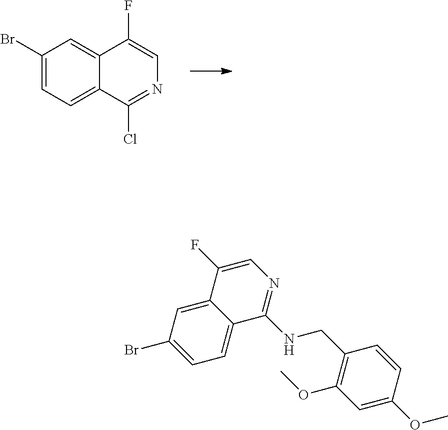 [SMILES: Fc1cnc(Cl)c2ccc(Br)cc12 ; COc1ccc(CNc2ncc(F)c3cc(Br)ccc23)c(OC)c1]

Following General Method 1c, 6-bromo-1-chloro-4-fluoroisoquinoline (6.88 g, 26.4 mmol) was reacted with 2,4-dimethoxybenzylamine (5.95 mL, 39.6 mmol) in 1-methyl-2-pyrrolidinone (100 mL) at 100° C. for 48 h. The crude product was purified by flash chromatography (Silica, 0-20% EtOAc in Pet. Ether) to give the product (3.2 g, 31% yield) as an off-white solid. [M−H]⁻=389.2

¹H NMR (500 MHz, DMSO) δ 8.35 (dd, J=9.0, 2.2 Hz, 1H), 7.98 (d, J=2.0 Hz, 1H), 7.90-7.70 (m, 3H), 7.07 (d, J=8.3 Hz, 1H), 6.55 (d, J=2.4 Hz, 1H), 6.41 (dd, J=8.5, 2.4 Hz, 1H), 4.56 (d, J=5.5 Hz, 2H), 3.82 (s, 3H), 3.72 (s, 3H).

¹⁹F NMR (471 MHz, DMSO) δ −157.4 (s).

### Intermediate 29

### 5-Bromo-N-(2,4-dimethoxybenzyl)-4-fluoroisoquinolin-1-amine

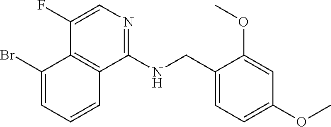 [SMILES: COc1ccc(CNc2ncc(F)c3c(Br)cccc23)c(OC)c1]

### 5-Bromo-1-chloro-4-fluoroisoquinoline

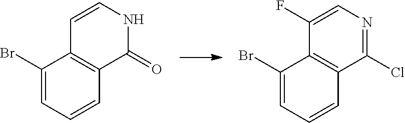 [SMILES: O=c1[nH]ccc2c(Br)cccc12 ; Fc1cnc(Cl)c2cccc(Br)c12]

A solution of 5-bromo-2H-isoquinolin-1-one (9.0 g, 40.2 mmol) and Selectfluor (17.1 g, 48.2 mmol) in MeCN (120 mL) and MeOH (120 mL) were heated at 50° C. for 3 h. The reaction mixture was evaporated and reacted using General Method 11, in 1,2-dichloroethane (200 mL) using benzyltriethylammonium chloride (915 mg, 4.0 mmol) and phosphorus oxychloride (45 mL) at 90° C. for 24 h. The reaction mixture was evaporated and the residue partitioned between DCM (500 mL) and water (500 mL). The organic layer was washed with water (300 mL) and brine (300 mL), dried (MgSO₄) and evaporated. The crude was purified by flash chromatography (Silica, 0-30% EtOAc in Pet. Ether) to give the product as a cream solid (5.70 g, 55% yield) [M+H]⁺=261.9

¹H NMR (500 MHz, CDCl₃) δ 8.39-8.33 (m, 1H), 8.23 (d, J=4.0 Hz, 1H), 8.12-8.06 (m, 1H), 7.57 (t, J=8.0 Hz, 1H).

### 5-Bromo-N-(2,4-dimethoxybenzyl)-4-fluoroisoquinolin-1-amine

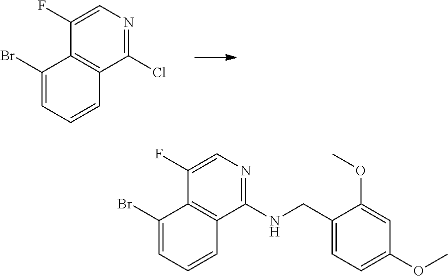 [SMILES: Fc1cnc(Cl)c2cccc(Br)c12 ; COc1ccc(CNc2ncc(F)c3c(Br)cccc23)c(OC)c1]

Following General Method 1c, 5-bromo-1-chloro-4-fluoroisoquinoline (5.70 g, 21.9 mmol) was reacted with 2,4-dimethoxybenzylamine (4.93 mL, 32.8 mmol) in 1-methyl-2-pyrrolidinone (80 mL) at 100° C. for 48 h. The crude product was purified by flash chromatography (Silica, 0-30% EtOAc in Pet. Ether) to give the product as a white solid (1.05 g, 12% yield).

¹H NMR (500 MHz, DMSO) δ 8.43 (dd, J=8.1, 2.3 Hz, 1H), 8.06 (dd, J=7.6, 0.9 Hz, 1H), 7.89 (d, J=5.1 Hz, 1H), 7.81 (t, J=5.6 Hz, 1H), 7.49 (t, J=8.0 Hz, 1H), 7.05 (d, J=8.3 Hz, 1H), 6.56 (d, J=2.4 Hz, 1H), 6.41 (dd, J=8.4, 2.4 Hz, 1H), 4.57 (d, J=5.5 Hz, 2H), 3.82 (s, 3H), 3.72 (s, 3H). ¹⁹F NMR (471 MHz, DMSO) δ −149.9 (s) [M−H]⁻=389.2

### Intermediate 30

### 4-Bromo-2-chloro-1H-pyrrolo[2,3-b]pyridine

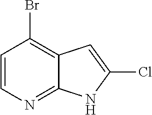 [SMILES: Clc1cc2c(Br)ccnc2[nH]1]

### 1-(Benzenesulfonyl)-4-bromopyrrolo[2,3-b]pyridine

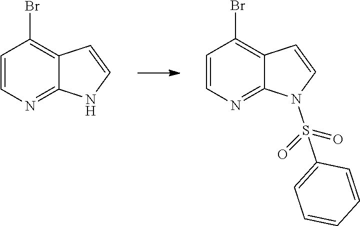

To a solution of 4-bromo-1H-pyrrolo[2,3-b]pyridine (5.00 g, 25.4 mmol) in DCM (130 mL) was added benzenesulfonyl chloride (4.86 mL, 38.1 mmol), 4-dimethylaminopyridine (310 mg, 2.54 mmol) and TEA (10.6 mL, 76.13 mmol). The reaction mixture was stirred at room temperature for 2 h. Upon completion the reaction mixture was concentrated under reduced pressure. The crude product was suspended in DCM (50 mL) and concentrated onto silica. The material was purified via flash chromatography (silica, 0-50% EtOAc in Pet. Ether) to afford the product (8.39 g, 98% yield) as a pale yellow solid.

[M+H]⁺=338.9

### 1-(Benzenesulfonyl)-4-bromo-2-chloropyrrolo[2,3-b]pyridine

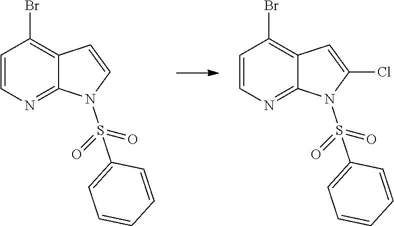

A dry flask was charged with 1-(benzenesulfonyl)-4-bromopyrrolo[2,3-b]pyridine (3.50 g, 10.4 mmol), sealed and purged with N₂ (g). THF (56 mL) was added and the mixture was cooled to −41° C. Lithium diisopropylamide (2M in THF) (12.5 mL, 24.9 mmol) was added slowly under N₂ (g). The mixture was stirred for 30 min at −41° C. before benzenesulfonyl chloride (2.65 mL, 20.8 mmol) was added. The reaction mixture was stirred for 2.5 h at −41° C. The reaction mixture was quenched with water (35 mL) and diluted with EtOAc (70 mL). The layers were separated and the aqueous layer was extracted with EtOAc (2×20 mL). Organic layers were combined and washed with brine (10 mL), dried (MgSO₄), filtered and concentrated in vacuo. Flash chromatography (silica, 0-60% EtOAc in Pet. Ether) afforded the product (3.92 g, 71% yield) as a pale yellow solid.

[M+H]⁺=372.9

### 4-Bromo-2-chloro-1H-pyrrolo[2,3-b]pyridine

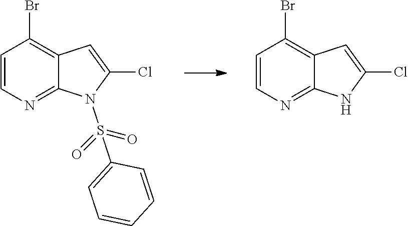

1-(Benzenesulfonyl)-4-bromo-2-chloropyrrolo[2,3-b]pyridine (3.92 g, 7.38 mmol) was taken up in 1,4-dioxane (20 mL) and NaOtBu (1.66 g, 14.8 mmol) was added. The reaction mixture was stirred at 80° C. for 2 h. The reaction mixture was diluted with EtOAc (10 mL) and washed with brine (10 mL). Layers were separated and the organic layer was dried (MgSO₄), filtered and concentrated in vacuo. The crude material was purified via flash chromatography (silica, 0-25% EtOAc in Pet. Ether). The product was triturated with Et₂O, taken up in EtOAc and concentrated in vacuo to afford the product (1.03 g, 60%) as a light beige solid.

[M+H]⁺=232.9

¹H NMR (CDCl₃, 400 MHz) δ 6.47 (1H, s), 7.32 (1H, d, J=5.3 Hz), 8.11 (1H, d, J=5.3 Hz).

### Intermediate 31

### N1-(2,4-Dimethoxybenzyl)isoquinoline-1,5-diamine

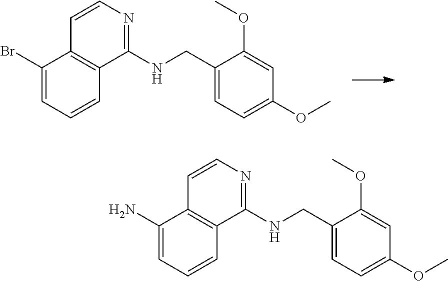 [SMILES: COc1ccc(CNc2nccc3c(Br)cccc23)c(OC)c1 ; COc1ccc(CNc2nccc3c(N)cccc23)c(OC)c1]

A mixture of 5-bromo-N-(2,4-dimethoxybenzyl)isoquinolin-1-amine (4.00 g, 10.7 mmol), 2,2,2-trifluoroacetamide (1.82 g, 16.1 mmol), copper(I) iodide (204 mg, 1.07 mmol), K₂CO₃ (2.96 g, 21.4 mmol) and DMF (189 mg, 241 μL, 2.14 mmol) was taken up in anhydrous 1,4-dioxane (10.6 mL) and the mixture purged with N₂ then heated to 75° C. for 24 h. MeOH (30 mL) and water (30 mL) were added and the mixture heated at 75° C. for 3.5 h. Organic solvents were removed under vacuum and the residue partitioned between EtOAc (50 mL) and water (50 mL). The aqueous layer was extracted with EtOAc (2×50 mL) and the combined organics washed with brine (50 mL), dried (MgSO₄), filtered and concentrated in vacuo. Flash chromatography (silica, 0-50% EtOAc/Iso-Hexanes then 0-5% (0.7M NH₃ in MeOH) in DCM) afforded the product (1.74 g, 52%).

[M+H]⁺=310.2

¹H NMR (d6 DMSO, 500 MHz) δ 3.71 (3H, s), 3.82 (3H, s), 4.58 (2H, d, J=5.7 Hz), 5.60 (2H, s), 6.39 (1H, dd, J=8.4, 2.4 Hz), 6.55 (1H, d, J=2.4 Hz), 6.77 (1H, dd, J=7.6, 0.9 Hz), 6.98-7.05 (2H, m), 7.17 (1H, t, J=7.9 Hz), 7.33 (1H, t, J=5.8 Hz), 7.42 (1H, d, J=8.3 Hz), 7.68 (1H, d, J=6.0 Hz).

### Intermediate 32

### 8-Methyl-3-(trifluoromethyl)-5,6,7,8-tetrahydro-[1,2,4]triazolo[4,3-a]pyrazine

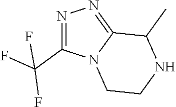

### tert-Butyl 3-(trifluoromethyl)-5,6-dihydro-[1,2,4]triazolo[4,3-a]pyrazine-7(8H)-carboxylate

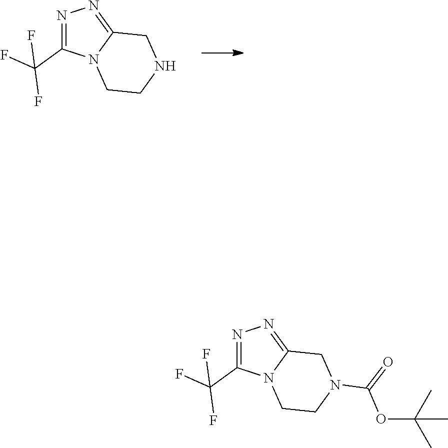 [SMILES: FC(F)(F)c1nnc2n1CCNC2 ; CC(C)(C)OC(=O)N1CCn2c(nnc2C(F)(F)F)C1]

3-(Trifluoromethyl)-5,6,7,8-tetrahydro-[1,2,4]triazolo[4,3-a]pyrazine (2.12 g, 11.0 mmol) was dissolved in DCM (20 mL) followed by the addition of Boc₂O (3.61 g, 16.5 mmol). The mixture was stirred for 18 h. The mixture was concentrated under reduced pressure. Flash chromatography (Silica, 0-70% EtOAc/Iso-Hexanes) afforded the product (2.66 g, 81%) as a white solid.

[M+H]⁺=293.2

¹H NMR (d6 DMSO, 500 MHz) δ 1.44 (9H, s), 3.83 (2H, t, J=5.5 Hz), 4.17 (2H, t, J=5.5 Hz), 4.77 (2H, s).

### tert-Butyl 8-methyl-3-(trifluoromethyl)-5,6-dihydro-[1,2,4]triazolo[4,3-a]pyrazine-7(8H)-carboxylate

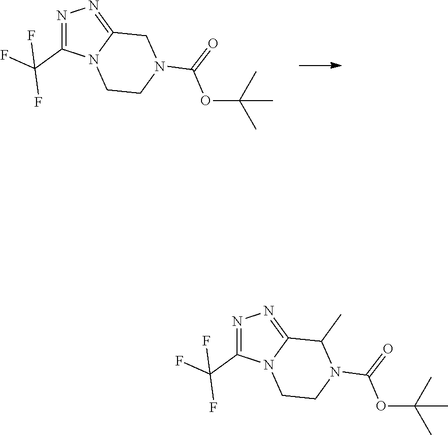 [SMILES: CC(C)(C)OC(=O)N1CCn2c(nnc2C(F)(F)F)C1 ; CC1c2nnc(C(F)(F)F)n2CCN1C(=O)OC(C)(C)C]

tert-Butyl 3-(trifluoromethyl)-5,6-dihydro-[1,2,4]triazolo[4,3-a]pyrazine-7(8H)-carboxylate (3.4 g, 12 mmol) was dissolved in toluene (40 mL). Tetramethylethylenediamine (1.5 g, 1.9 mL, 13 mmol) was added. The reaction mixture was cooled to −78° C. under N₂ (g). ⁺BuLi (6.5 mL, 2.5 M in hexanes, 16 mmol) was added and the mixture stirred at −78° C. for 10 min. Mel (8.3 g, 3.6 mL, 58 mmol) was added and the mixture stirred for a further 10 min before being warmed to rt and stirred for 18 h. The mixture was diluted with NH₄Cl(aq) (20 mL) and extracted with EtOAc (3×25 mL). Organic layers were combined, dried (MgSO₄), filtered and concentrated in vacuo. Flash chromatography (Silica, 0-85% EtOAc in isohexane) afforded the product (2.2 g, 62%).

[M+H]⁺=307.2

### 8-Methyl-3-(trifluoromethyl)-5,6,7,8-tetrahydro-[1,2,4]triazolo[4,3-a]pyrazine

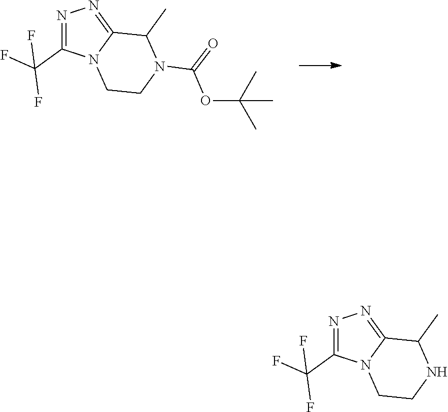 [SMILES: CC1c2nnc(C(F)(F)F)n2CCN1C(=O)OC(C)(C)C ; CC1NCCn2c1nnc2C(F)(F)F]

tert-Butyl 8-methyl-3-(trifluoromethyl)-5,6-dihydro-[1,2,4]triazolo[4,3-a]pyrazine-7(8H)-carboxylate (397 mg, 1.30 mmol) was dissolved in DCM (9 mL). TFA (2 mL) was added. The mixture was stirred for 1.5 h before being concentrated under reduced pressure. The crude product was loaded onto SCX with MeOH, washed with MeOH and eluted with 0.7M NH₃ in MeOH. Concentration in vacuo afforded the product (201 mg, 75%) as a yellow oil.

[M+H]⁺=207.2

¹H NMR (CDCl3, 500 MHz) δ 1.68 (3H, d, J=6.7 Hz), 3.22 (1H, ddd, J=13.6, 10.3, 4.6 Hz), 3.47 (1H, ddd, J=13.5, 4.8, 2.6 Hz), 4.04-4.18 (2H, m), 4.29 (1H, q, J=6.6 Hz).

### Intermediate 33

### (6-(3-(Difluoromethyl)-5,6-dihydro-[1,2,4]triazolo[4,3-a]pyrazin-7(8H)-yl)pyridin-3-yl)methanamine

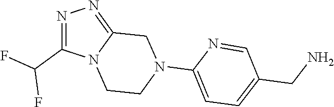 [SMILES: NCc1ccc(N2CCn3c(nnc3C(F)F)C2)nc1]

### 6-(3-(Difluoromethyl)-5,6-dihydro-[1,2,4]triazolo[4,3-a]pyrazin-7(8H)-yl)nicotinonitrile

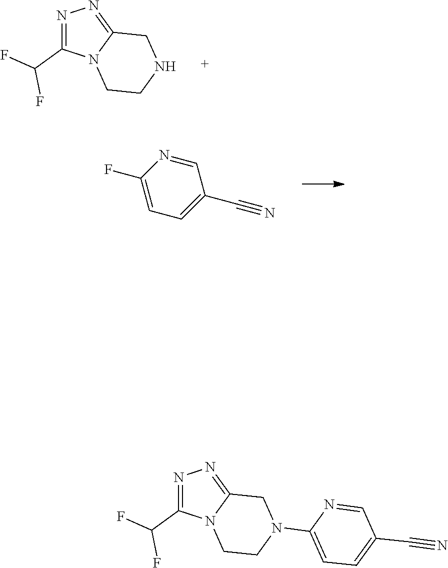 [SMILES: FC(F)c1nnc2n1CCNC2 ; N#Cc1ccc(F)nc1 ; N#Cc1ccc(N2CCn3c(nnc3C(F)F)C2)nc1]

A solution of 2-fluoropyridine-5-carbonitrile (229 mg, 1.88 mmol) in MeCN (3 mL) was treated with a solution of 3-(difluoromethyl)-5,6,7,8-tetrahydro-[1,2,4]triazolo[4,3-a]pyrazine (343 mg, 1.97 mmol) and DIPEA (497 mg, 3.84 mmol) in MeCN (3 mL). The mixture was heated at 85° C. for 20 h. After cooling, solvents were removed under vacuum. Flash chromatography (Silica, 0-3.5% (0.7M NH₃ in MeOH) in DCM) afforded the product (366 mg, 70% yield) as a white solid.

[M+H]⁺=277.2

¹H NMR (500 MHz, DMSO-d6) 4.17-4.29 (4H, m), 5.09 (2H, s), 7.18 (1H, dd, J=9.1, 0.8 Hz), 7.37 (1H, t, J=51.8 Hz), 8.00 (1H, dd, J=9.0, 2.3 Hz), 8.60 (1H, dd, J=2.4, 0.7 Hz) (6-(3-(Difluoromethyl)-5,6-dihydro-[1,2,4]triazolo[4,3-a]pyrazin-7(8H)-yl)pyridin-3-yl)methanamine

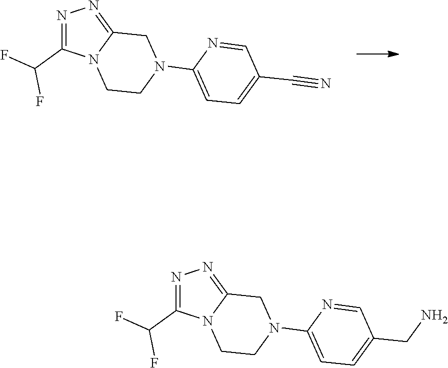 [SMILES: N#Cc1ccc(N2CCn3c(nnc3C(F)F)C2)nc1 ; NCc1ccc(N2CCn3c(nnc3C(F)F)C2)nc1]

6-(3-(Difluoromethyl)-5,6-dihydro-[1,2,4]triazolo[4,3-a]pyrazin-7(8H)-yl)nicotinonitrile (363 mg, 1.31 mmol) was reduced according to General Method 3a, using Raney Ni for 4.5 h. Solvents were removed under vacuum. Flash chromatography (Silica, 0-14% (0.7M NH₃ in MeOH) in DCM) afforded the product (217 mg, 58% yield) as a white solid.

[M+H]⁺=281.2

¹H NMR (500 MHz, DMSO-d6) 1.75 (2H, s), 3.60 (2H, s), 4.06 (2H, t, J=5.5 Hz), 4.20 (2H, t, J=5.5 Hz), 4.92 (2H, s), 7.05 (1H, d, J=8.6 Hz), 7.35 (1H, t, J=51.9 Hz), 7.62 (1H, dd, J=8.7, 2.4 Hz), 8.10 (1H, d, J=2.3 Hz)

### SPECIFIC EXAMPLES OF THE PRESENT INVENTION

### Example Number 2185

### N6-(4-(((1-methylpiperidin-4-yl)oxy)methyl)benzyl)isoquinoline-1,6-diamine

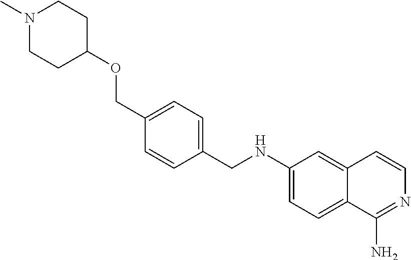 [SMILES: CN1CCC(OCc2ccc(CNc3ccc4c(N)nccc4c3)cc2)CC1]

### Tert-butyl (6-((4-(((1-methylpiperidin-4-yl)oxy)methyl)benzyl)amino)isoquinolin-1-yl)carbamate

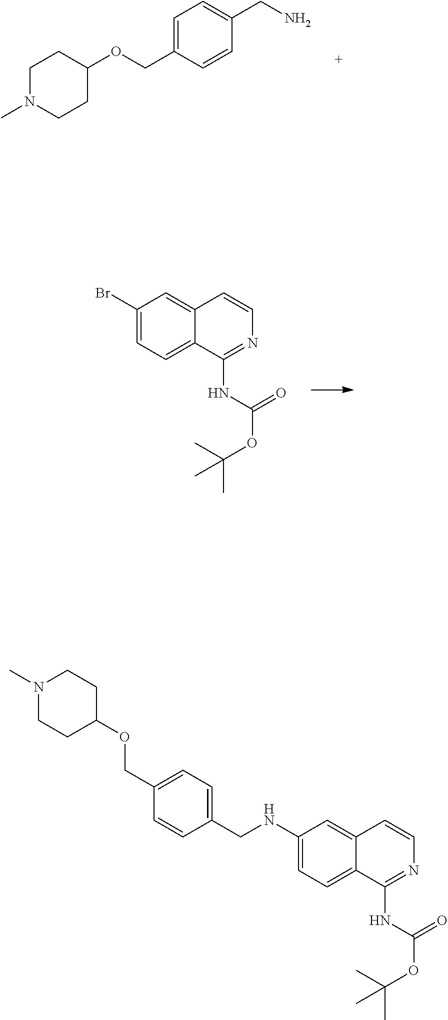 [SMILES: CN1CCC(OCc2ccc(CN)cc2)CC1 ; CC(C)(C)OC(=O)Nc1nccc2cc(Br)ccc12 ; CN1CCC(OCc2ccc(CNc3ccc4c(NC(=O)OC(C)(C)C)nccc4c3)cc2)CC1]

Tert-butyl(6-bromoisoquinolin-1-yl)carbamate (130 mg, 0.40 mmol) was reacted with (4-(((1-methylpiperidin-4-yl)oxy)methyl)phenyl)methanamine (85 mg, 0.36 mmol) using General Method 4 and NaOtBu (80 mg, 0.83 mmol) in THF (3 mL) at 60° C. for 1 h. The reaction mixture was quenched, concentrated and purified by flash chromatography (Silica, 0-20% (0.7 M NH₃ in MeOH) in DCM) to obtain the product (170 mg, 93% yield) as a colourless solid.

[M+H]⁺=477.3

### N6-(4-(((1-methylpiperidin-4-yl)oxy)methyl)benzyl)isoquinoline-1,6-diamine

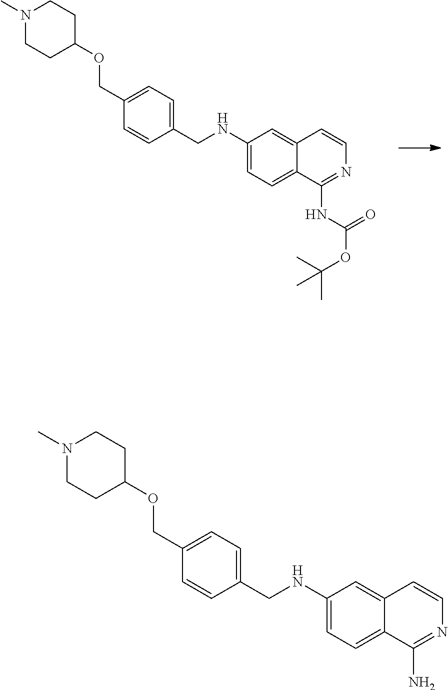 [SMILES: CN1CCC(OCc2ccc(CNc3ccc4c(NC(=O)OC(C)(C)C)nccc4c3)cc2)CC1 ; CN1CCC(OCc2ccc(CNc3ccc4c(N)nccc4c3)cc2)CC1]

Boc deprotection of tert-butyl (6-((4-(((1-methylpiperidin-4-yl)oxy)methyl)benzyl)amino)isoquinolin-1-yl)carbamate (155 mg, 0.33 mmol) was carried out using General Method 7b. The crude product was purified by flash chromatography (Silica, 0-20% (0.7M NH₃ in MeOH) in DCM) to afford the product (82 mg, 66% yield) as a colourless solid.

[M+H]⁺=377.2

¹H NMR (500 MHz, DMSO-d6) δ 1.43-1.53 (2H, m), 1.77-1.87 (2H, m), 1.94-2.02 (2H, m), 2.12 (3H, s), 2.55-2.61 (2H, m), 3.29-3.36 (1H, m), 4.35 (2H, d, J=5.9 Hz), 4.45 (2H, s), 6.28 (2H, s), 6.46 (1H, d, J=2.4 Hz), 6.52 (1H, d, J=5.8 Hz), 6.75 (1H, t, J=6.0 Hz), 6.87 (1H, dd, J=9.0, 2.4 Hz), 7.25-7.30 (2H, m), 7.33-7.37 (2H, m), 7.53 (1H, d, J=5.8 Hz), 7.84 (1H, d, J=9.1 Hz)

### Example Number 1003

### N7-(4-((1-methylpiperidin-4-yl)oxy)benzyl)isoquinoline-1,7-diamine

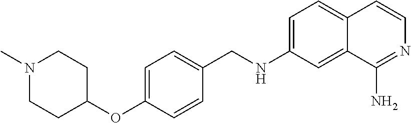 [SMILES: CN1CCC(Oc2ccc(CNc3ccc4ccnc(N)c4c3)cc2)CC1]

### N7-(4-((1-methylpiperidin-4-yl)oxy)benzyl)isoquinoline-1,7-diamine

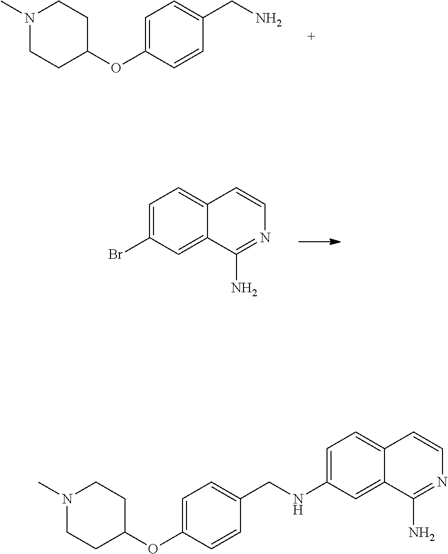 [SMILES: CN1CCC(Oc2ccc(CN)cc2)CC1 ; Nc1nccc2ccc(Br)cc12 ; CN1CCC(Oc2ccc(CNc3ccc4ccnc(N)c4c3)cc2)CC1]

Following General Method 4, 7-bromoisoquinolin-1-amine (51 mg, 0.23 mmol) was reacted with (4-((1-methylpiperidin-4-yl)oxy)phenyl)methanamine (50 mg, 0.23 mmol) using NaOtBu (2M in THF) (0.23 mL, 0.46 mmol) in anhydrous 1,4-dioxane (3 mL) at 50° C. for 2 h. The reaction was quenched, concentrated and purified by flash chromatography (Silica, 0-20% (0.7M NH₃ in MeOH) in DCM) to afford the product which was further purified by prep HPLC (5-50% in basic mobile phase) to obtain the product (5 mg, 6% yield) as a light brown solid.

[M+H]⁺=363.2

¹H NMR (500 MHz, DMSO-d6) δ 1.53-1.64 (2H, m), 1.86-1.94 (2H, m), 2.10-2.18 (5H, m), 2.56-2.62 (2H, m), 4.26-4.35 (3H, m), 6.26 (2H, s), 6.31 (1H, t, J=5.9 Hz), 6.71 (1H, d, J=5.7 Hz), 6.89-6.92 (2H, m), 7.01 (1H, d, J=2.2 Hz), 7.11 (1H, dd, J=8.8, 2.2 Hz), 7.32-7.35 (2H, m), 7.40 (1H, d, J=8.8 Hz), 7.49 (1H, d, J=5.7 Hz).

### Example Number 3253

### N6-((1-((1-methylpiperidin-4-yl)methyl)-1H-pyrazol-4-yl)methyl)isoquinoline-1,6-diamine

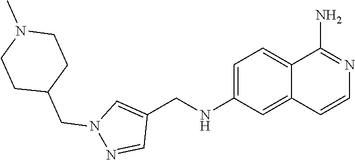 [SMILES: CN1CCC(Cn2cc(CNc3ccc4c(N)nccc4c3)cn2)CC1]

### Tert-butyl 4-((4-cyano-1H-pyrazol-1-yl)methyl)piperidine-1-carboxylate

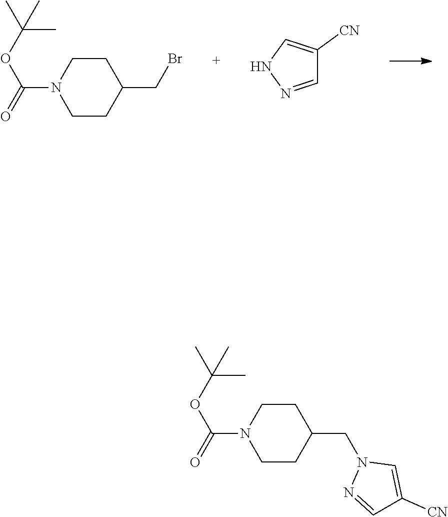 [SMILES: CC(C)(C)OC(=O)N1CCC(CBr)CC1 ; N#Cc1cn[nH]c1 ; CC(C)(C)OC(=O)N1CCC(Cn2cc(C#N)cn2)CC1]

Following General Method 5b, a solution of tert-butyl 4-(bromomethyl)piperidine-1-carboxylate (1.20 g, 4.30 mmol), 1H-pyrazole-4-carbonitrile (250 mg, 2.69 mmol) and K₂CO₃ (921 mg, 6.66 mmol) was stirred in NMP (5 mL) in the microwave at 130° C. for 2 h. The reaction was quenched with MeOH (5 mL) and diluted with water (50 mL). The product was extracted into TBME (2×50 mL) and washed with brine (50 mL). The organic layer was dried (Na₂SO₄), filtered and concentrated to afford the product (756 mg, 89% yield) as a white solid.

¹H NMR (500 MHz, DMSO-d6) δ 1.00-1.10 (2H, m), 1.39 (9H, s), 1.94-2.05 (1H, m), 2.08-2.21 (2H, m), 2.65-2.75 (2H, m), 3.91 (2H, s), 4.08 (2H, d, J=7.1 Hz), 8.07 (1H, s), 8.55 (1H, s).

### 1-(piperidin-4-ylmethyl)-1H-pyrazole-4-carbonitrile

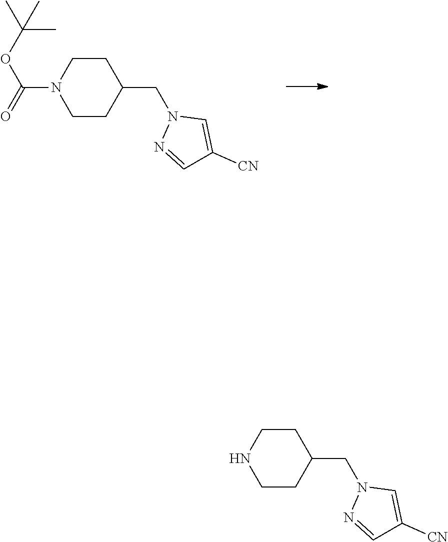 [SMILES: CC(C)(C)OC(=O)N1CCC(Cn2cc(C#N)cn2)CC1 ; N#Cc1cnn(CC2CCNCC2)c1]

Boc deprotection of tert-butyl 4-((4-cyano-1H-pyrazol-1-yl)methyl)piperidine-1-carboxylate (900 mg, 3.10 mmol) was carried out using General Method 7b to afford the product (517 mg, 76% yield) as a light orange oil.

[M+H]⁺=191.1

¹H NMR (500 MHz, DMSO-d6) δ 0.99-1.12 (2H, m), 1.30-1.40 (2H, m), 1.83-1.94 (1H, m), 2.34-2.44 (2H, m), 2.87-2.98 (2H, m), 4.04 (2H, d, J=7.2 Hz), 8.06 (1H, s), 8.55 (1H, s). NH not observed.

### 1-((1-Methylpiperidin-4-yl)methyl)-1H-pyrazole-4-carbonitrile

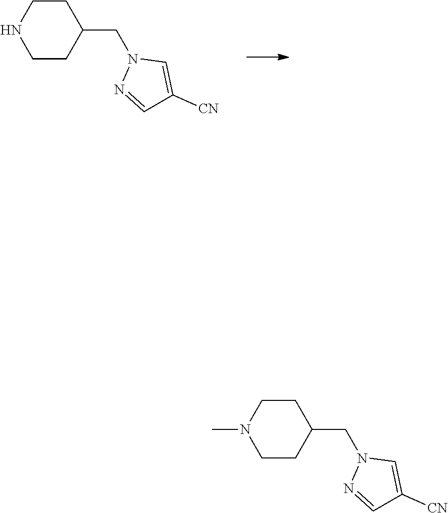

Following General Method 9, 1-(piperidin-4-ylmethyl)-1H-pyrazole-4-carbonitrile (498 mg, 2.62 mmol) was reacted with paraformaldehyde (314 mg, 10.44 mmol) in DCM (6.5 mL) and DMF (0.5 mL) at 40° C. for 5 h. The crude product was purified by flash chromatography (Silica, 0-10% (0.7M NH₃ in MeOH) in DCM) to afford the product (352 mg, 63% yield) as a clear colourless oil.

[M+H]⁺=205.3

¹H NMR (500 MHz, DMSO-d6) δ 1.12-1.26 (2H, m), 1.34-1.45 (2H, m), 1.68-1.82 (3H, m), 2.12 (3H, s), 2.68-2.76 (2H, m), 4.06 (2H, d, J=7.2 Hz), 8.06 (1H, s), 8.55 (1H, s).

### (1-((1-Methylpiperidin-4-yl)methyl)-1H-pyrazol-4-yl)methanamine

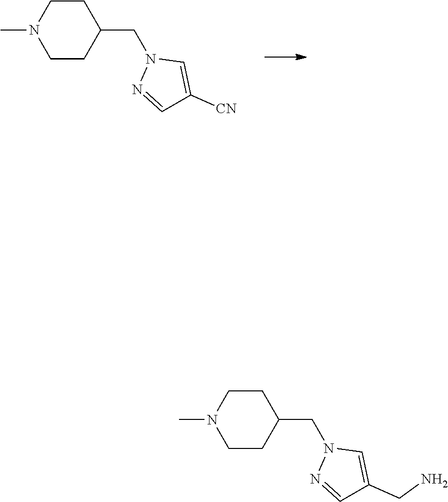

The nitrile, 1-((1-methylpiperidin-4-yl)methyl)-1H-pyrazole-4-carbonitrile (200 mg, 0.98 mmol) was reduced following General Method 3a using a Raney Ni CatCart for 4 h. The crude residue was dissolved in MeOH passed directly through an SCX. The product was eluted with a solution of 7M NH₃ in MeOH (180 mg, 50% yield) and isolated as a colourless oil.

[M+H]⁺=209.4

¹H NMR (500 MHz, DMSO-d6) δ 1.10-1.23 (2H, m), 1.37-1.46 (2H, m), 1.62-1.71 (1H, m), 1.73-1.80 (2H, m), 2.11 (3H, s), 2.67-2.75 (2H, m), 3.56 (2H, s), 3.91 (2H, d, J=7.2, 4.0 Hz), 7.32 (1H, d, J=2.7 Hz), 7.51 (1H, s). NH₂ hidden under water peak.

### N6-((1-((1-methylpiperidin-4-yl)methyl)-1H-pyrazol-4-yl)methyl)isoquinoline-1,6-diamine

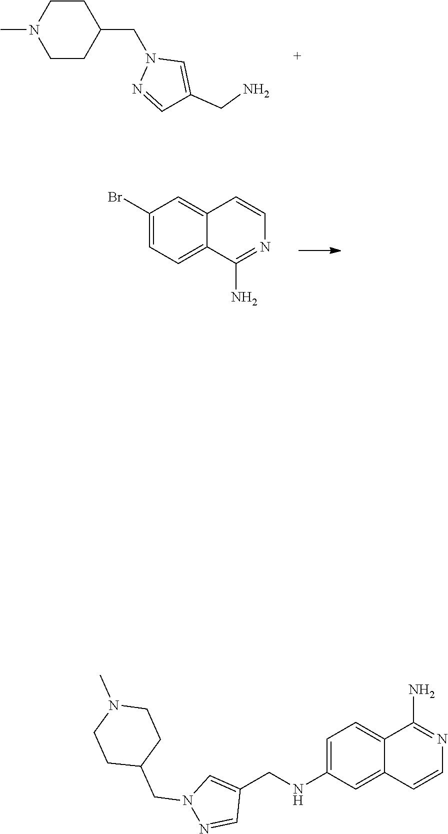 [SMILES: CN1CCC(Cn2cc(CN)cn2)CC1 ; Nc1nccc2cc(Br)ccc12 ; CN1CCC(Cn2cc(CNc3ccc4c(N)nccc4c3)cn2)CC1]

Following General Method 4, (1-((1-methylpiperidin-4-yl)methyl)-1H-pyrazol-4-yl)methanamine (40 mg, 0.19 mmol) was reacted with 6-bromoisoquinolin-1-amine (43 mg, 0.19 mmol) using NaOtBu (37 mg, 0.38 mmol) in anhydrous 1,4-dioxane (3 mL) 50° C. for 18 h. After elution through an SCX, the crude product was further purified by prep HPLC (10-40% in basic mobile phase) to obtain the product (9.0 mg, 13% yield) as a white solid.

[M+H]⁺=351.4

¹H NMR (500 MHz, DMSO-d6) δ 1.10-1.22 (2H, m), 1.37-1.44 (2H, m), 1.62-1.78 (3H, m), 2.11 (3H, s), 2.66-2.73 (2H, m), 3.93 (2H, d, J=7.2 Hz), 4.15 (2H, d, J=5.4 Hz), 6.28 (2H, s), 6.36 (1H, t, J=5.5 Hz), 6.54 (1H, d, J=2.3 Hz), 6.59 (1H, d, J=5.8 Hz), 6.86 (1H, dd, J=9.0, 2.3 Hz), 7.42 (1H, s), 7.56 (1H, d, J=5.8 Hz), 7.66 (1H, s), 7.83 (1H, d, J=9.0 Hz).

### Example Number 3254

### N6-((1-(2-(1-methylpiperidin-4-yl)ethyl)-1H-pyrazol-4-yl)methyl)isoquinoline-1,6-diamine

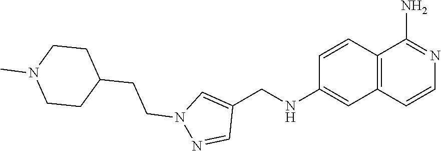 [SMILES: CN1CCC(CCn2cc(CNc3ccc4c(N)nccc4c3)cn2)CC1]

### Tert-butyl (6-(((1-(2-(1-methylpiperidin-4-yl)ethyl)-1H-pyrazol-4-yl)methyl)amino)isoquinolin-1-yl)carbamate

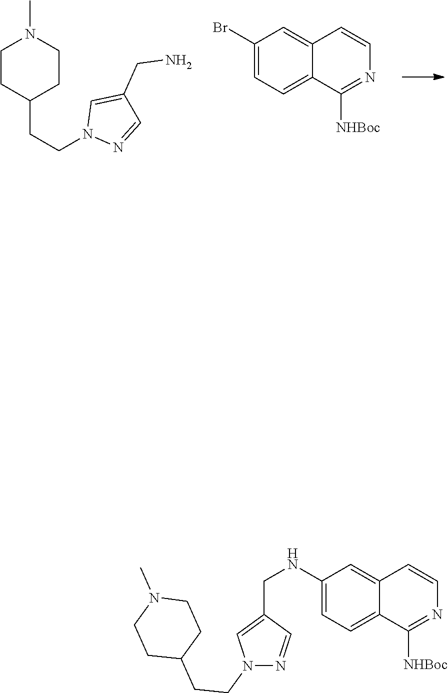 [SMILES: CN1CCC(CCn2cc(CN)cn2)CC1 ; CC(C)(C)OC(=O)Nc1nccc2cc(Br)ccc12 ; CN1CCC(CCn2cc(CNc3ccc4c(NC(=O)OC(C)(C)C)nccc4c3)cn2)CC1]

Following General Method 4, (1-(2-(1-methylpiperidin-4-yl)ethyl)-1H-pyrazol-4-yl)methanamine (75 mg, 0.34 mmol) was reacted with tert-butyl (6-bromoisoquinolin-1-yl)carbamate (120 mg, 0.37 mmol) in the presence of NaOtBu (80 mg, 0.83 mmol) in THF (3 mL) at 60° C. for 1 h. The crude product was purified by flash chromatography (Silica, 0-20% (0.7M NH₃ in MeOH) in DCM) to afford the product (121 mg, 69% yield) as a colourless solid.

[M+H]⁺=465.3

¹H NMR (500 MHz, DMSO-d6) 1.18-1.43 (3H, m), 1.47 (9H, s), 1.64-1.77 (2H, m), 1.77-1.88 (2H, m), 2.66-2.82 (6H, m), 3.30-3.38 (1H, m), 4.11 (2H, t, J=7.1 Hz), 4.21 (2H, d, J=5.4 Hz), 6.70 (1H, s), 6.80-6.96 (1H, m), 6.95-7.12 (1H, m), 7.12-7.30 (1H, m), 7.46 (1H, s), 7.73 (1H, s), 7.75-8.01 (2H, m). 8.17-8.30 (1H, m)

### N6-((1-(2-(1-methylpiperidin-4-yl)ethyl)-1H-pyrazol-4-yl)methyl)isoquinoline-1,6-diamine

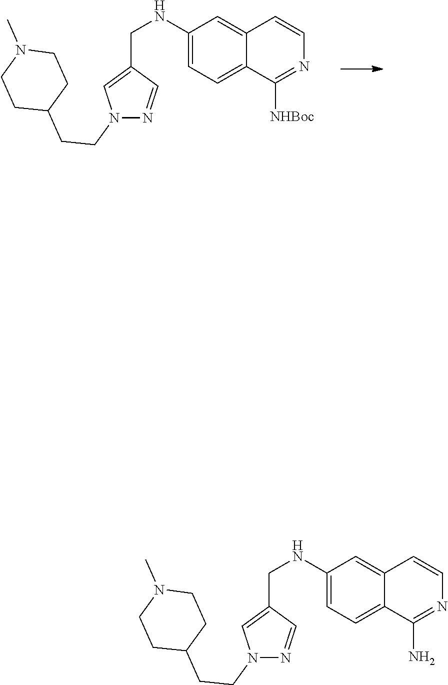 [SMILES: CN1CCC(CCn2cc(CNc3ccc4c(NC(=O)OC(C)(C)C)nccc4c3)cn2)CC1 ; CN1CCC(CCn2cc(CNc3ccc4c(N)nccc4c3)cn2)CC1]

Tert-butyl (6-(((1-(2-(1-methylpiperidin-4-yl)ethyl)-1H-pyrazol-4-yl)methyl)amino)isoquinolin-1-yl)carbamate (100 mg, 0.22 mmol) was deprotected according to General Method 7b. After elution through an SCX, the crude product was purified by flash chromatography (silica, 0-20% (0.7M NH₃ in MeOH) in DCM) to afford the product (35 mg, 43% yield) as a colourless solid.

[M+H]⁺=365.2

¹H NMR (500 MHz, DMSO-d6) 1.04-1.18 (2H, m), 1.54-1.74 (7H, m), 2.09 (3H, s), 2.62-2.70 (2H, m), 4.07 (2H, t, J=7.2 Hz), 4.15 (2H, d, J=5.3 Hz), 6.28 (2H, s), 6.37 (1H, t, J=5.4 Hz), 6.54 (1H, d, J=2.3 Hz), 6.59 (1H, d, J=5.9 Hz), 6.86 (1H, dd, J=9.0, 2.3 Hz), 7.42 (1H, s), 7.55 (1H, d, J=5.8 Hz), 7.69 (1H, s), 7.83 (1H, d, J=9.1 Hz)

### Example Number 1004

### 3-Chloro-N-(4-((1-methylpiperidin-4-yl)oxy)benzyl)-1-((2-(trimethylsilyl)ethoxy)methyl)-1H-pyrrolo[2,3-b]pyridin-5-amine

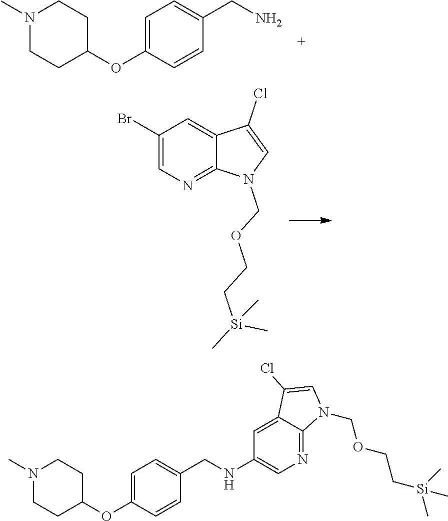

Following General Method 4, (4-((1-methylpiperidin-4-yl)oxy)phenyl)methanamine (61 mg, 0.28 mmol) was reacted with 5-bromo-3-chloro-1-((2-(trimethylsilyl)ethoxy)methyl)-1H-pyrrolo[2,3-b]pyridine (100 mg, 0.28 mmol), in the presence of 2M NaOtBu in THF (0.28 mL, 0.56 mmol) at rt for 1 h. After elution through an SCX, the crude product was purified by flash chromatography (Silica, 0-20% (1% NH₃ in MeOH) in DCM) to afford the product (65 mg, 38% yield) as a yellow oil.

[M+H]⁺=501.2

¹H NMR (500 MHz, DMSO-d6) δ −0.10 (9H, s), 0.76-0.82 (2H, m), 1.55-1.65 (2H, m), 1.86-1.94 (2H, m), 2.13-2.24 (5H, m), 2.57-2.67 (2H, m), 3.42-3.53 (2H, m), 4.25 (2H, d, J=6.0 Hz), 4.27-4.36 (1H, m), 5.48 (2H, s), 6.20 (1H, t, J=6.0 Hz), 6.87-6.93 (3H, m), 7.28-7.31 (2H, m), 7.63 (1H, s), 7.94 (1H, d, J=2.6 Hz)

### 3-Chloro-N-(4-((1-methylpiperidin-4-yl)oxy)benzyl)-1H-pyrrolo[2,3-b]pyridin-5-amine

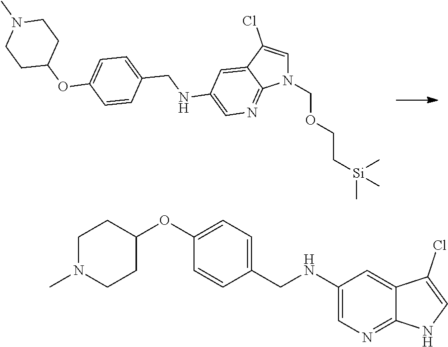

To a solution of 3-chloro-N-(4-((1-methylpiperidin-4-yl)oxy)benzyl)-1-((2-(trimethylsilyl)ethoxy)methyl)-1H-pyrrolo[2,3-b]pyridin-5-amine (40 mg, 0.08 mmol) in DCM (1 mL) that was cooled in an ice/water bath was added TFA (0.10 mL, 1.30 mmol) dropwise and the mixture was stirred for 1 h. The reaction was allowed to warm to rt and stirred for 18 h. The reaction was diluted with MeOH (3 mL) and passed directly through an SCX and washed with MeOH (30 mL). The required compound was eluted with 7M NH₃ in MeOH (50 mL) and concentrated. The crude product was purified by flash chromatography (Silica, 0-20% (1% NH₃ in MeOH) in DCM) then by prep HPLC (5-50% MeCN in water, basic mobile phase) to obtain the product (7.0 mg, 23% yield) as a pale yellow solid.

[M+H]⁺=371.1

¹H NMR (500 MHz, DMSO-d6) δ 1.57-1.67 (2H, m), 1.88-1.94 (2H, m), 2.15-2.24 (5H, m), 2.59-2.67 (2H, m), 4.23 (2H, d, J=6.0 Hz), 4.28-4.36 (1H, m), 6.06 (1H, t, J=6.0 Hz), 6.87 (1H, d, J=2.6 Hz), 6.89-6.93 (2H, m), 7.28-7.32 (2H, m), 7.41 (1H, d, J=2.8 Hz), 7.88 (1H, d, J=2.6 Hz), 11.44 (1H, d, J=1.8 Hz)

### Example Number 3255

### N5-((1-(2-(1-methylpiperidin-4-yl)ethyl)-1H-pyrazol-4-yl)methyl)isoquinoline-1,5-diamine

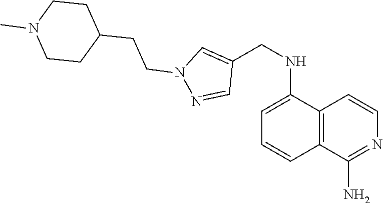 [SMILES: CN1CCC(CCn2cc(CNc3cccc4c(N)nccc34)cn2)CC1]

### Tert-butyl (5-(((1-(2-(1-methylpiperidin-4-yl)ethyl)-1H-pyrazol-4-yl)methyl)amino)isoquinolin-1-yl)carbamate

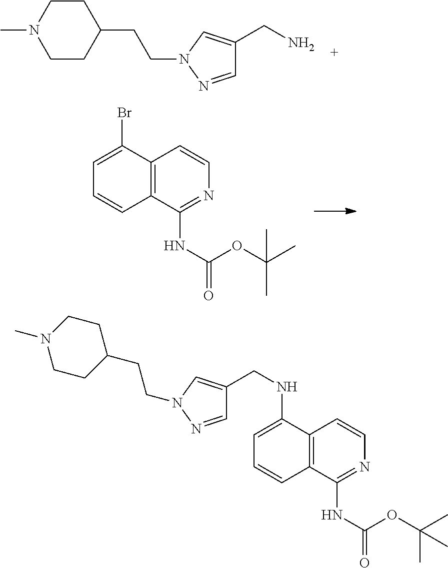 [SMILES: CN1CCC(CCn2cc(CN)cn2)CC1 ; CC(C)(C)OC(=O)Nc1nccc2c(Br)cccc12 ; CN1CCC(CCn2cc(CNc3cccc4c(NC(=O)OC(C)(C)C)nccc34)cn2)CC1]

Following General Method 4, (1-(2-(1-methylpiperidin-4-yl)ethyl)-1H-pyrazol-4-yl)methanamine (75 mg, 0.34 mmol) was reacted with tert-butyl (5-bromoisoquinolin-1-yl)carbamate (120 mg, 0.37 mmol), in the presence of NaOtBu (80 mg, 0.83 mmol) in THF (5 mL) at 60° C. for 5 h. After quenching the reaction mixture with AcOH (40 μL, 0.70 mmol) for 5 min, 1M NH₃ in MeOH (20 mL) was added and the reaction mixture was concentrated. The crude product was purified by flash chromatography (Silica, 0-20% (0.7 M NH₃ in MeOH) in DCM) to afford the product (35 mg, 22% yield) as an off-white solid.

[M+H]⁺=465.2

### N5-((1-(2-(1-methylpiperidin-4-yl)ethyl)-1H-pyrazol-4-yl)methyl)isoquinoline-1,5-diamine

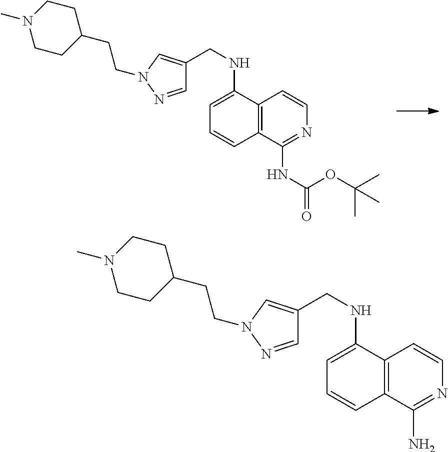

Tert-butyl (5-(((1-(2-(1-methylpiperidin-4-yl)ethyl)-1H-pyrazol-4-yl)methyl)amino)isoquinolin-1-yl)carbamate (35 mg, 0,075 mmol) was deprotected using General Method 7b. The crude product was purified by flash chromatography (Silica, 0-20% (0.7M NH₃ in MeOH) in DCM) to afford the product (5.0 mg, 17% yield) as a red solid.

[M+H]⁺=365.2

¹H NMR (500 MHz, DMSO-d6) δ 1.04-1.21 (3H, m), 1.57-1.68 (4H, m), 1.81-1.94 (2H, m), 2.20 (3H, s), 2.74-2.81 (2H, m), 4.05 (2H, t, J=7.2 Hz), 4.26 (2H, d, J=5.6 Hz), 6.32 (1H, t, J=5.8 Hz), 6.49 (2H, s), 6.63 (1H, d, J=7.7 Hz), 7.12 (1H, d, J=6.1 Hz), 7.18 (1H, t, J=8.0 Hz), 7.32 (1H, d, J=8.3 Hz), 7.40 (1H, d, J=0.7 Hz), 7.64 (1H, s), 7.71 (1H, d, J=6.1 Hz)

### Example Number 2186

### N5-(4-(((1-methylpiperidin-4-yl)oxy)methyl)benzyl)isoquinoline-1,5-diamine

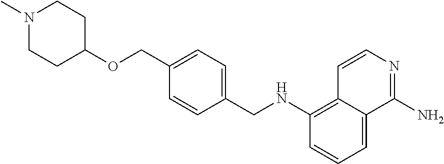 [SMILES: CN1CCC(OCc2ccc(CNc3cccc4c(N)nccc34)cc2)CC1]

### Tert-butyl (5-((4-(((1-methylpiperidin-4-yl)oxy)methyl)benzyl)amino)isoquinolin-1-yl)carbamate

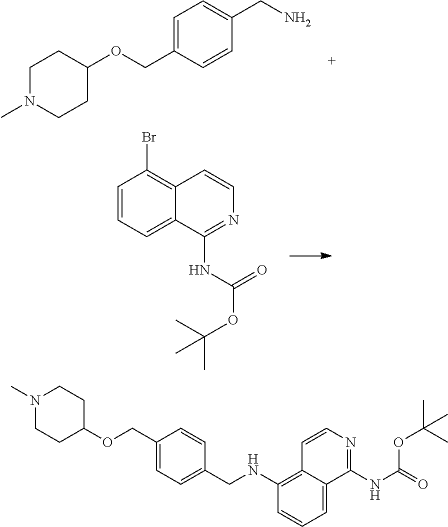 [SMILES: CN1CCC(OCc2ccc(CN)cc2)CC1 ; CC(C)(C)OC(=O)Nc1nccc2c(Br)cccc12 ; CN1CCC(OCc2ccc(CNc3cccc4c(NC(=O)OC(C)(C)C)nccc34)cc2)CC1]

Following General Method 4, (4-(((1-methylpiperidin-4-yl)oxy)methyl)phenyl)methanamine (60 mg, 0.26 mmol) was reacted with tert-butyl (5-bromoisoquinolin-1-yl)carbamate (90 mg, 0.28 mmol) and NaOtBu (50 mg, 0.52 mmol) in THF (3 mL) at 60° C. for 3 h. After quenching the reaction mixture and concentrating in vacuo, the crude product was purified by flash chromatography (Silica, 0-20% (0.7M NH₃ in MeOH) in DCM) to afford the product (50 mg, 40% yield) as an off-white solid.

[M+H]⁺=477.3

### N5-(4-(((1-methylpiperidin-4-yl)oxy)methyl)benzyl)isoquinoline-1,5-diamine

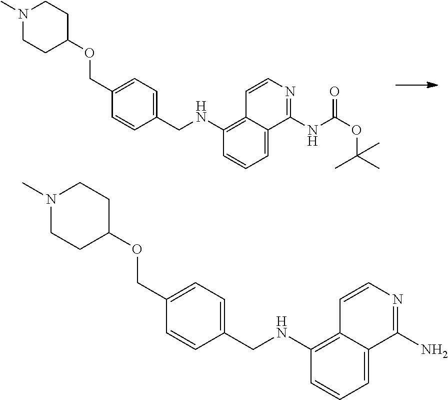

Tert-butyl (5-((4-(((1-methylpiperidin-4-yl)oxy)methyl)benzyl)amino)isoquinolin-1-yl)carbamate (50 mg, 0.10 mmol) was deprotected according to General Method 7b. The crude product was purified by flash chromatography (Silica, 0-20% (0.7M NH₃ in MeOH) in DCM) to afford the product (21 mg, 49% yield) as a yellow solid.

[M+H]⁺=377.2

¹H NMR (500 MHz, DMSO-d6) 1.43-1.57 (2H, m), 1.79-1.89 (2H, m), 2.02-2.14 (2H, m), 2.17 (3H, s), 2.59-2.66 (2H, m), 3.37-3.39 (1H, m), 4.44 (2H, s), 4.45 (2H, s), 6.43 (1H, d, J=7.7 Hz), 6.50 (2H, s), 6.76 (1H, t, J=6.0 Hz), 7.07-7.13 (1H, m), 7.21 (1H, d, J=6.1 Hz), 7.23-7.28 (2H, m), 7.30 (1H, d, J=8.3 Hz), 7.32-7.36 (2H, m), 7.75 (1H, d, J=6.1 Hz)

### Example Number 2189

### N6-(4-(((1-methylpiperidin-4-yl)oxy)methyl)benzyl)isoquinoline-3,6-diamine

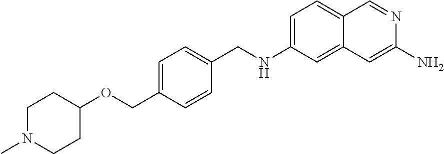 [SMILES: CN1CCC(OCc2ccc(CNc3ccc4cnc(N)cc4c3)cc2)CC1]

### Tert-butyl (6-((4-(((1-methylpiperidin-4-yl)oxy)methyl)benzyl)amino)isoquinolin-3-yl)carbamate

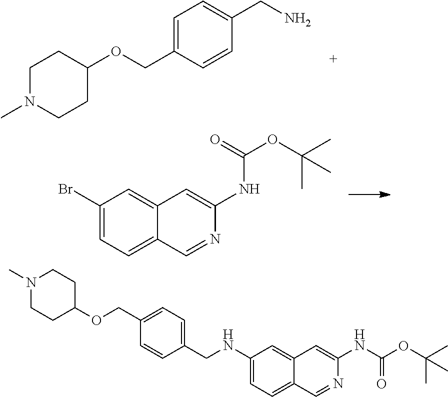 [SMILES: CN1CCC(OCc2ccc(CN)cc2)CC1 ; CC(C)(C)OC(=O)Nc1cc2cc(Br)ccc2cn1 ; CN1CCC(OCc2ccc(CNc3ccc4cnc(NC(=O)OC(C)(C)C)cc4c3)cc2)CC1]

Following General Method 4, tert-butyl (6-bromoisoquinolin-3-yl)carbamate (69 mg, 0.21 mmol) was reacted with (4-(((1-methylpiperidin-4-yl)oxy)methyl)phenyl)methanamine (50 mg, 0.21 mmol) in the presence of 2M NaOtBu in THF (0.2 mL, 0.4 mmol) in THF (3 mL) at 60° C. for 1 h. After elution through an SCX the crude product was purified by flash chromatography (Silica, 0-20% (0.7M NH₃ in MeOH) in DCM) to afford the product (64 mg, 62% yield) as an off-white solid.

[M+H]⁺=477.3

### N6-(4-(((1-methylpiperidin-4-yl)oxy)methyl)benzyl)isoquinoline-3,6-diamine

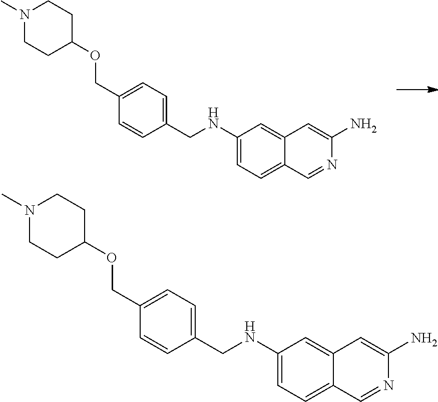 [SMILES: CN1CCC(OCc2ccc(CNc3ccc4cnc(N)cc4c3)cc2)CC1 ; CN1CCC(OCc2ccc(CNc3ccc4cnc(N)cc4c3)cc2)CC1]

Tert-butyl (6-((4-(((1-methylpiperidin-4-yl)oxy)methyl)benzyl)amino)isoquinolin-3-yl)carbamate (64 mg, 0.13 mmol) was deprotected using General Method 7b. After elution through an SCX, the crude product was purified by prep HPLC (5-50% MeCN in water, basic mobile phase) to afford the product (23 mg, 44% yield) as a pale pink solid.

[M+H]⁺=377.2

¹H NMR (500 MHz, DMSO-d6) δ 1.43-1.52 (2H, m), 1.80-1.86 (2H, m), 1.96-2.02 (2H, m), 2.12 (3H, s), 2.54-2.61 (2H, m), 3.34-3.37 (1H, m), 4.33 (2H, d, J=5.9 Hz), 4.45 (2H, s), 5.48 (2H, s), 6.17-6.23 (2H, m), 6.67 (1H, dd, J=8.9, 2.2 Hz), 6.75 (1H, t, J=5.9 Hz), 7.28 (2H, d, J=8.1 Hz), 7.35 (2H, d, J=8.1 Hz), 7.44 (1H, d, J=8.9 Hz), 8.37 (1H, s).

### Example Number 2187

### N6-(4-(((1-methylpiperidin-4-yl)oxy)methyl)benzyl)quinoline-2,6-diamine

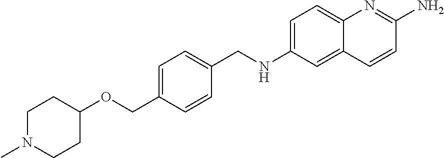 [SMILES: CN1CCC(OCc2ccc(CNc3ccc4nc(N)ccc4c3)cc2)CC1]

### N6-(4-(((1-methylpiperidin-4-yl)oxy)methyl)benzyl)quinoline-2,6-diamine

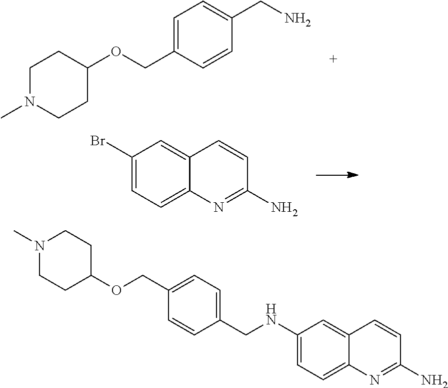

Following General Method 4, 6-bromoquinolin-2-amine (106 mg, 0.47 mmol) was reacted with (4-(((1-methylpiperidin-4-yl)oxy)methyl)phenyl)methanamine (111 mg, 0.47 mmol), in the presence of 2M NaOtBu in THF (0.48 mL, 0.96 mmol) in THF (3 mL) at 60° C. for 2 h. After elution through an SCX the crude product was purified by prep HPLC (5-50% MeCN in water, basic mobile phase) to afford the product (11 mg, 6% yield) as an off-white solid.

[M+H]⁺=377.2

¹H NMR (500 MHz, DMSO-d6) δ 1.44-1.52 (2H, m), 1.80-1.87 (2H, m), 1.95-2.02 (2H, m), 2.12 (3H, s), 2.55-2.62 (2H, m), 3.33-3.37 (1H, m), 4.30 (2H, d, J=6.0 Hz), 4.45 (2H, s), 5.35 (2H, s), 6.24 (1H, t, J=6.0 Hz), 6.50 (1H, s), 6.59 (1H, d, J=2.3 Hz), 7.09 (1H, dd, J=8.9, 2.3 Hz), 7.28 (2H, d, J=8.0 Hz), 7.32 (1H, d, J=8.9 Hz), 7.37 (2H, d, J=8.0 Hz), 8.44 (1H, s).

### Example Number 2190

### N6-methyl-N6-(4-(((1-methylpiperidin-4-yl)oxy)methyl)benzyl)isoquinoline-1,6-diamine

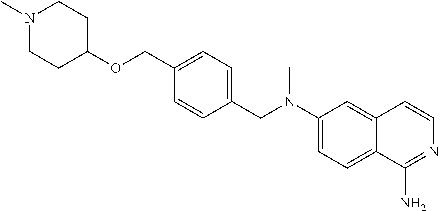 [SMILES: CN1CCC(OCc2ccc(CN(C)c3ccc4c(N)nccc4c3)cc2)CC1]

### Tert-butyl (6-(methyl(4-(((1-methylpiperidin-4-yl)oxy)methyl)benzyl)amino)isoquinolin-1-yl)carbamate

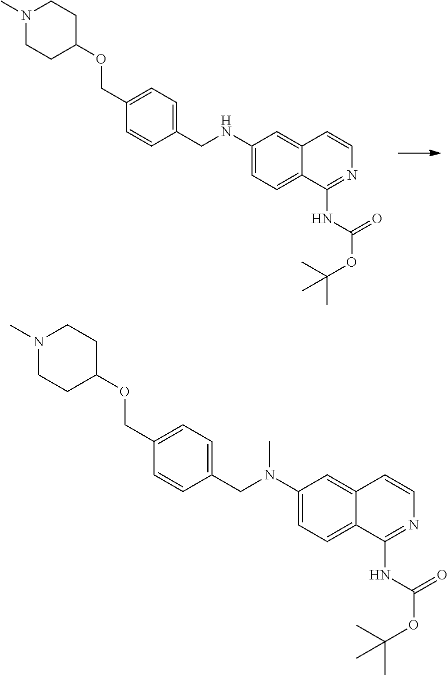 [SMILES: CN1CCC(OCc2ccc(CNc3ccc4c(NC(=O)OC(C)(C)C)nccc4c3)cc2)CC1 ; CN1CCC(OCc2ccc(CN(C)c3ccc4c(NC(=O)OC(C)(C)C)nccc4c3)cc2)CC1]

Following General Method 9, tert-butyl (6-((4-(((1-methylpiperidin-4-yl)oxy)methyl)benzyl)amino)isoquinolin-1-yl)carbamate (95 mg, 0.20 mmol), was reacted with paraformaldehyde (24 mg, 0.81 mmol) in DCM (3 mL) and DMF (0.3 mL) at 40° C. for 3 h. The reaction mixture was diluted in 0.7M NH₃/MeOH (10 mL) and concentrated under reduced pressure. The crude product was purified by flash chromatography (Silica, 0-15% (0.7M NH₃ in MeOH) in DCM) to afford the product (50 mg, 36% yield) as a colourless gum.

[M+H]⁺=491.5

### N6-methyl-N6-(4-(((1-methylpiperidin-4-yl)oxy)methyl)benzyl)isoquinoline-1,6-diamine

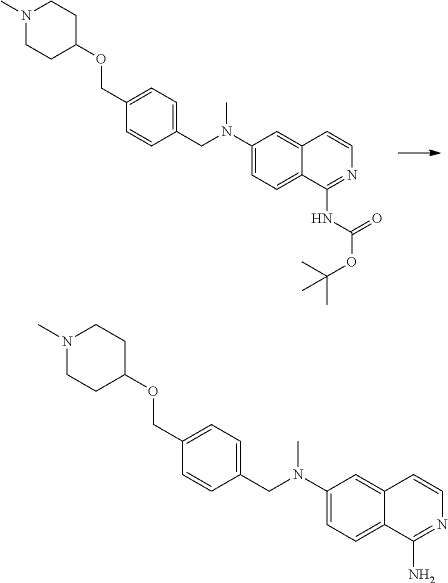 [SMILES: CN1CCC(OCc2ccc(CN(C)c3ccc4c(NC(=O)OC(C)(C)C)nccc4c3)cc2)CC1 ; CN1CCC(OCc2ccc(CN(C)c3ccc4c(N)nccc4c3)cc2)CC1]

Tert-butyl (6-(methyl (4-(((1-methyl piperidin-4-yl)oxy)methyl)benzyl)amino)isoquinolin-1-yl)carbamate (46 mg, 0.09 mmol) was deprotected using General Method 7b at rt for 48 h. After elution through an SCX, the crude product was purified by flash chromatography (Silica, 0-20% (0.7 M NH₃ in MeOH) in DCM) to afford the product (15 mg, 39% yield) as a cream solid.

[M+H]⁺=391.5

¹H NMR (500 MHz, DMSO-d6) δ 1.42-1.53 (2H, m), 1.78-1.86 (2H, m), 1.95-2.05 (2H, m), 2.12 (3H, s), 2.53-2.62 (2H, m), 3.10 (3H, s), 3.29-3.33 (1H, m), 4.44 (2H, s), 4.70 (2H, s), 6.40 (2H, s), 6.63 (1H, d, J=5.9 Hz), 6.69 (1H, d, J=2.6 Hz), 7.04 (1H, dd, J=9.3, 2.7 Hz),7.16-7.21 (2H, m),7.23-7.28 (2H, m), 7.57 (1H, d, J=5.9 Hz), 7.94 (1H, d, J=9.2 Hz).

### Example Number 2191

### N6-(2-fluoro-4-(((1-methylpiperidin-4-yl)oxy)methyl)benzyl)isoquinoline-1,6-diamine

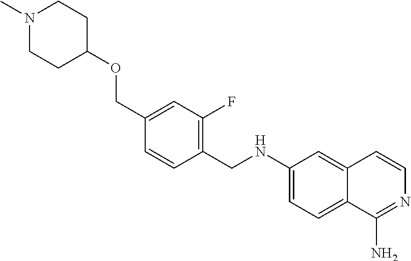 [SMILES: CN1CCC(OCc2ccc(CNc3ccc4c(N)nccc4c3)c(F)c2)CC1]

### Tert-butyl (6-((2-fluoro-4-(((1-methylpiperidin-4-yl)oxy)methyl)benzyl)amino)isoquinolin-1-yl)carbamate

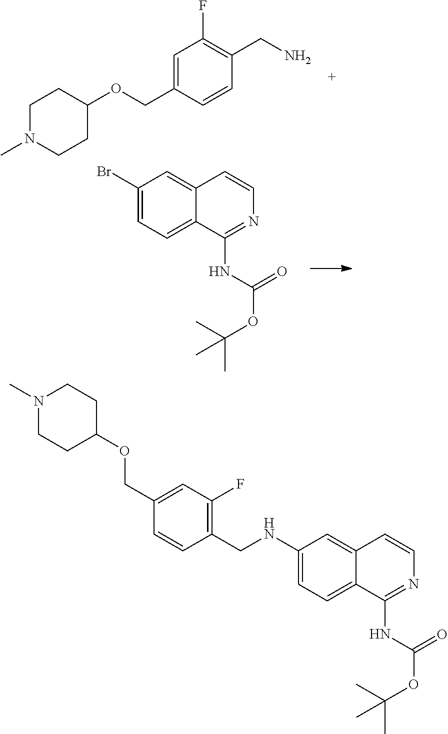

Following General Method 4, (6-bromoisoquinolin-1-yl)carbamate (141 mg, 0.44 mmol) was reacted with (2-fluoro-4-(((1-methylpiperidin-4-yl)oxy)methyl)phenyl)methanamine (110 mg, 0.44 mmol) and NaOtBu (84 mg, 0.87 mmol) in THF (3 mL) at 60° C. for 1 h. After quenching, the crude product was purified by flash chromatography (Silica, 0-20% (0.7M NH₃ in MeOH) in DCM) to obtain the product (172 mg, 72% yield) as a cream solid.

[M+H]⁺=495.5

### N6-(2-fluoro-4-(((1-methylpiperidin-4-yl)oxy)methyl)benzyl)isoquinoline-1,6-diamine

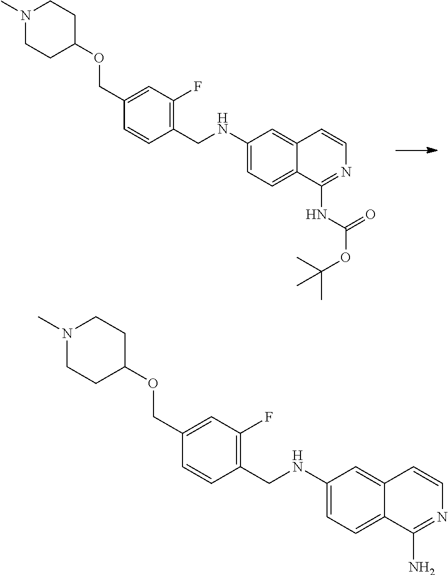 [SMILES: CN1CCC(OCc2ccc(CNc3ccc4c(NC(=O)OC(C)(C)C)nccc4c3)c(F)c2)CC1 ; CN1CCC(OCc2ccc(CNc3ccc4c(N)nccc4c3)c(F)c2)CC1]

Tert-butyl (6-((2-fluoro-4-(((1-methylpiperidin-4-yl)oxy)methyl)benzyl)amino)isoquinolin-1-yl)carbamate (140 mg, 0.25 mmol) was deprotected using General Method 7b. After elution through an SCX the crude product was purified by flash chromatography (Silica, 0-20% (0.7M NH₃ in MeOH) in DCM) to afford the product (96 mg, 93% yield) as a colourless solid.

[M+H]⁺=395.4

¹H NMR (500 MHz, DMSO-d6) δ 1.42-1.54 (2H, m), 1.79-1.87 (2H, m), 1.95-2.03 (2H, m), 2.12 (3H, s), 2.54-2.61 (2H, m), 3.34-3.37 (1H, m), 4.38 (2H, d, J=5.9 Hz), 4.47 (2H, s), 6.30 (2H, s), 6.47-6.50 (1H, m), 6.55 (1H, d, J=5.9 Hz), 6.67-6.72 (1H, m), 6.86-6.90 (1H, m), 7.10 (1H, d, J=7.9 Hz), 7.15 (1H, d, J=11.0 Hz), 7.34-7.39 (1H, m), 7.54 (1H, d, J=5.8 Hz), 7.86 (1H, d, J=9.0 Hz).

### Example Number 1005

### N6-((6-((1-methylpiperidin-4-yl)methoxy)pyridin-3-yl)methyl)isoquinoline-1,6-diamine

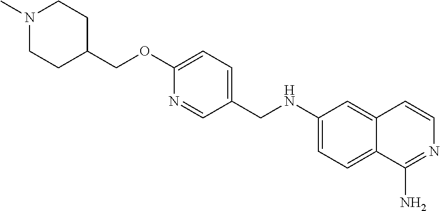 [SMILES: CN1CCC(COc2ccc(CNc3ccc4c(N)nccc4c3)cn2)CC1]

### Methyl (6-(((6-((1-methylpiperidin-4-yl)methoxy)pyridin-3-yl)methyl)amino)isoquinolin-1-yl)carbamate

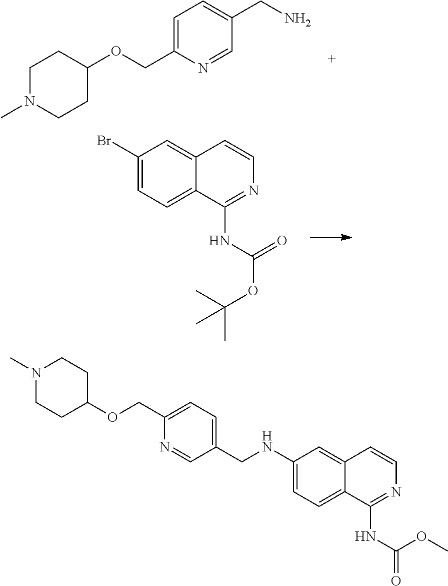

Following General Method 4, (6-((1-methylpiperidin-4-yl)methoxy)pyridin-3-yl)methanamine (75.0 mg, 0.32 mmol) was reacted with tert-butyl (6-bromoisoquinolin-1-yl)carbamate (103 mg, 0.32 mmol), in the presence of 1M KO^{t}Bu in 1,4-dioxane (0.64 mL, 0.64 mmol) in 1,4-dioxane (4 mL) at 60° C. for 1 h. The reaction mixture was concentrated and the crude product was purified by flash chromatography (Silica, 0-10% (0.7M NH₃ in MeOH) in DCM) to afford the product (104 mg, 49% yield) as an off white solid.

[M+H]⁺=436.5

### N6-((6-((1-methylpiperidin-4-yl)methoxy)pyridin-3-yl)methyl)isoquinoline-1,6-diamine

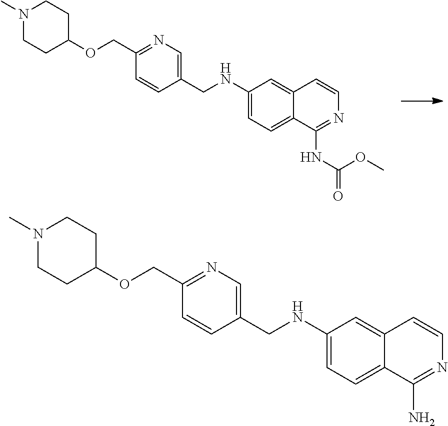

Methyl (6-(((6-((1-methylpiperidin-4-yl)methoxy)pyridin-3-yl)methyl)amino)isoquinolin-1-yl)carbamate (90 mg, 0.21 mmol) was deprotected using General Method 14a for 4 h. The reaction mixture was concentrated and the crude product was purified by flash chromatography (Silica, 2-20% (0.7M NH₃ in MeOH) in DCM). The product was lyophilised to afford the product (49 mg, 60% yield) as a white solid.

[M+H]⁺=378.4

¹H NMR (500 MHz, DMSO-d6) δ 1.19-1.34 (2H, m), 1.63-1.74 (3H, m), 1.83-1.92 (2H, m), 2.17 (3H, s), 2.74-2.82 (2H, m), 4.07 (2H, d, J=6.1 Hz), 4.29 (2H, d, J=5.8 Hz), 6.40 (2H, s), 6.54 (1H, d, J=2.4 Hz), 6.58 (1H, d, J=5.9 Hz), 6.68-6.74 (1H, m), 6.78 (1H, d, J=8.5 Hz), 6.87 (1H, dd, J=9.1, 2.4 Hz), 7.54 (1H, d, J=5.9 Hz), 7.70 (1H, dd, J=8.5, 2.5 Hz), 7.86 (1H, d, J=9.0 Hz), 8.17 (1H, d, J=2.4 Hz)

### Example Number 1006

### N6-((6-((5,6,7,8-tetrahydroimidazo[1,2-a]pyridin-7-yl)methoxy)pyridin-3-yl)methyl)isoquinoline-1,6-diamine

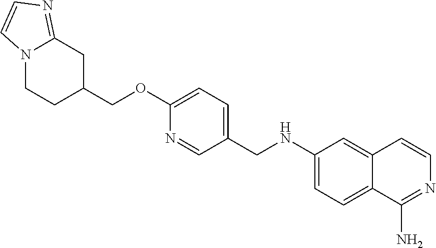 [SMILES: Nc1nccc2cc(NCc3ccc(OCC4CCn5ccnc5C4)nc3)ccc12]

### 6-((5,6,7,8-tetrahydroimidazo[1,2-a]pyridin-7-yl)methoxy)nicotinonitrile

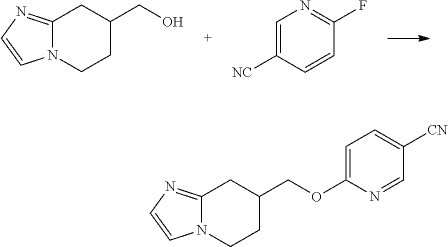 [SMILES: OCC1CCn2ccnc2C1 ; N#Cc1ccc(F)nc1 ; N#Cc1ccc(OCC2CCn3ccnc3C2)nc1]

Following General Method 1b, (5,6,7,8-tetrahydroimidazo[1,2-a]pyridin-7-yl)methanol (890 mg, 5.85 mmol) was reacted with 6-fluoronicotinonitrile (714 mg, 5.85 mmol) for 5 h. The crude reaction mixture was passed directly through an SCX. The SCX was washed with MeOH and the product was eluted with 7M NH₃ in MeOH. The crude product was purified by flash chromatography (Silica, 0-10% (0.7 NH₃ in MeOH) in DCM) to afford the product (723 mg, 44% yield) as a pale brown solid.

[M+H]⁺=255.3

¹H NMR (500 MHz, DMSO-d6) δ 1.68-1.80 (1H, m), 2.10-2.18 (1H, m), 2.35-2.45 (1H, m), 2.51-2.55 (1H, m), 2.94 (1H, ddd, J=16.2, 5.0, 1.5 Hz), 3.84-3.94 (1H, m), 4.04-4.13 (1H, m), 4.37 (2H, d, J=6.6 Hz), 6.81 (1H, d, J=1.2 Hz), 7.00 (1H, d, J=1.2 Hz), 7.06 (1H, dd, J=8.7, 0.8 Hz), 8.18 (1H, dd, J=8.7, 2.4 Hz), 8.71 (1H, dd, J=2.4, 0.8 Hz)

### Tert-butyl ((6-((5,6,7,8-tetrahydroimidazo[1,2-a]pyridin-7-yl)methoxy)pyridin-3-yl)methyl)carbamate

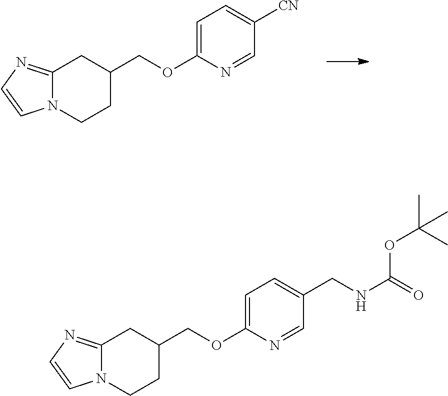 [SMILES: N#Cc1ccc(OCC2CCn3ccnc3C2)nc1 ; CC(C)(C)OC(=O)NCc1ccc(OCC2CCn3ccnc3C2)nc1]

Following general method 3d, the nitrile, 6-((5,6,7,8-tetrahydroimidazo[1,2-a]pyridin-7-I)methoxy)nicotinonitrile (400 mg, 1.57 mmol) was reduced in MeOH (14 mL) and THF (9.0 mL). After 18 h, water (2 mL) was added and the reaction mixture filtered, washing with THF (20 mL) and concentrated. The crude product was purified by chromatography (Silica, 0-10% (0.7M NH₃ in MeOH) in DCM) to afford the product (315 mg, 45% yield) as a sticky colourless gum.

[M+H]⁺=359.4

### (6-((5,6,7,8-Tetrahydroimidazo[1,2-a]pyridin-7-yl)methoxy)pyridin-3-yl)methanamine

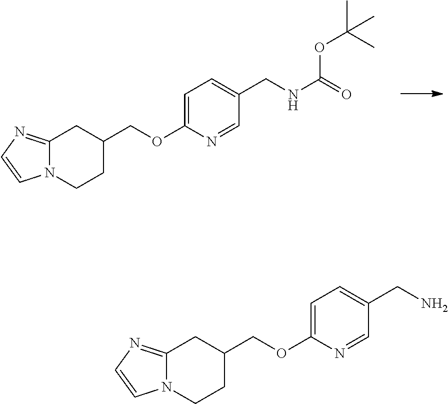 [SMILES: CC(C)(C)OC(=O)NCc1ccc(OCC2CCn3ccnc3C2)nc1 ; NCc1ccc(OCC2CCn3ccnc3C2)nc1]

Boc deprotection of tert-butyl ((6-((5,6,7,8-tetrahydroimidazo[1,2-a]pyridin-7-yl)methoxy)pyridin-3-yl)methyl)carbamate (170 mg, 0.47 mmol) was performed using General Method 7b. After elution through an SCX the product was isolated (124 mg, 90% yield) as a pale yellow oil.

[M+H]⁺=259.3

¹H NMR (500 MHz, DMSO-d6) 1.66-1.80 (1H, m), 2.08-2.18 (1H, m), 2.31-2.44 (1H, m), 2.47-2.50 (1H, m, obscured by DMSO), 2.93 (1H, ddd, J=16.3, 5.1, 1.5 Hz), 3.76 (2H, s), 3.83-3.96 (1H, m), 4.03-4.14 (1H, m), 4.26 (2H, dd, J=6.6, 1.4 Hz), 6.81 (1H, d, J=1.3 Hz), 6.84 (1H, d, J=8.5 Hz), 6.99 (1H, d, J=1.2 Hz), 7.73 (1H, dd, J=8.5, 2.5 Hz), 8.11 (1H, d, J=2.4 Hz), (NH₂ not observed)

### Methyl (6-(((6-((5,6,7,8-tetrahydroimidazo[1,2-a]pyridin-7-yl)methoxy)pyridin-3-yl)methyl)amino)isoquinolin-1-yl)carbamate

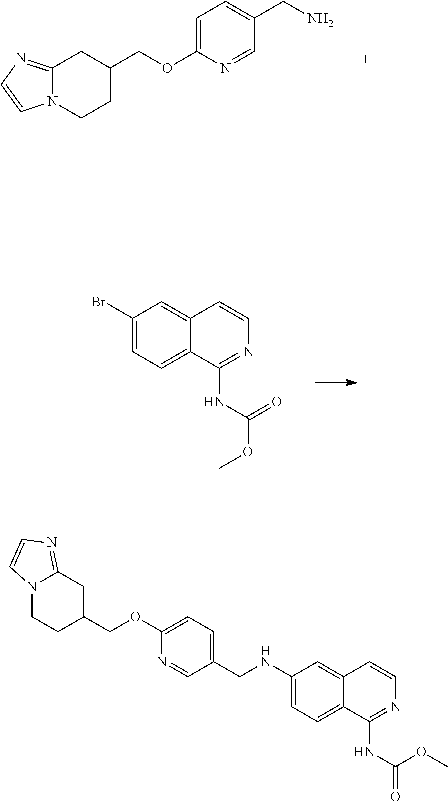 [SMILES: NCc1ccc(OCC2CCn3ccnc3C2)nc1 ; COC(=O)Nc1nccc2cc(Br)ccc12 ; COC(=O)Nc1nccc2cc(NCc3ccc(OCC4CCn5ccnc5C4)nc3)ccc12]

Following General Method 4, (6-((5,6,7,8-tetrahydroimidazo[1,2-a]pyridin-7-yl)methoxy)pyridin-3-yl)methanamine (108 mg, 0.42 mmol) was reacted with methyl (6-bromoisoquinolin-1-yl)carbamate (118 mg, 0.418 mmol) and NaOtBu (80 mg, 0.83 mmol) in THF (8 mL) at 60° C. for 1 h. After quenching the reaction mixture, the crude product was purified by flash chromatography (Silica, 0-20% (0.7M NH₃ in MeOH) in DCM) to afford the product (60 mg, 28% yield) as an off-white solid.

[M+H]⁺=459.4

### N6-((6-((5,6,7,8-tetrahydroimidazo[1,2-a]pyridin-7-yl)methoxy)pyridin-3-yl)methyl)isoquinoline-1,6-diamine

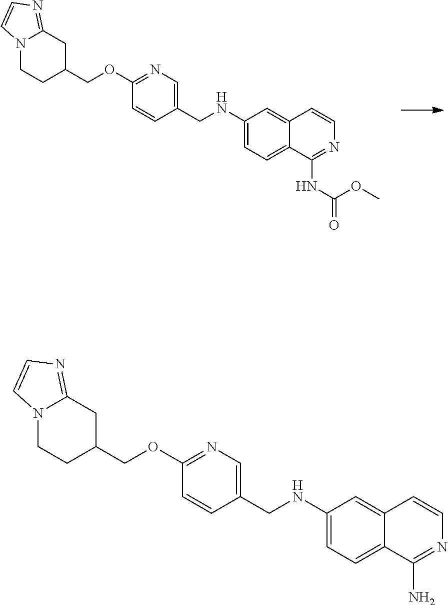 [SMILES: COC(=O)Nc1nccc2cc(NCc3ccc(OCC4CCn5ccnc5C4)nc3)ccc12 ; Nc1nccc2cc(NCc3ccc(OCC4CCn5ccnc5C4)nc3)ccc12]

Deprotection of methyl (6-(((6-((5,6,7,8-tetrahydroimidazo[1,2-a]pyridin-7-yl)methoxy)pyridin-3-yl)methyl)amino)isoquinolin-1-yl)carbamate (57 mg, 0.12 mmol) was performed using General Method 14a for 18 h. Following quenching, elution through an SCX and lyophilisation the product was isolated (45 mg, 89% yield) as an off-white solid.

[M+H]⁺=401.5

¹H NMR (500 MHz, DMSO-d6) δ 1.67-1.78 (1H, m), 2.10-2.16 (1H, m), 2.31-2.41 (1H, m), 2.45-2.51 (1H, m, partially obscured by DMSO), 2.92 (1H, ddd, J=16.3, 5.0, 1.5 Hz), 3.84-3.92 (1H, m), 4.05-4.11 (1H, m), 4.25 (2H, d, J=6.6 Hz), 4.31 (2H, d, J=5.8 Hz), 6.52-6.58 (3H, m), 6.61 (1H, d, J=6.0 Hz), 6.77-6.82 (2H, m), 6.84 (1H, d, J=8.5 Hz), 6.89 (1H, dd, J=9.1, 2.4 Hz), 6.99 (1H, d, J=1.3 Hz), 7.53 (1H, d, J=6.0 Hz), 7.73 (1H, dd, J=8.5, 2.5 Hz), 7.89 (1H, d, J=9.0 Hz), 8.20 (1H, d, J=2.4 Hz).

### Examples 1023 and 1024 (Enantiomers)

### N6-((6-((5,6,7,8-tetrahydroimidazo[1,2-a]pyridin-7-yl)methoxy)pyridin-3-yl)methyl)isoquinoline-1,6-diamine

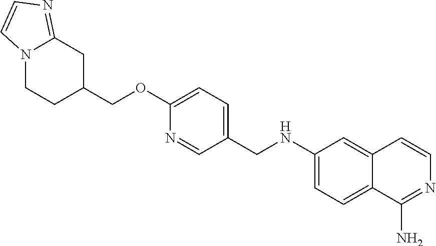 [SMILES: Nc1nccc2cc(NCc3ccc(OCC4CCn5ccnc5C4)nc3)ccc12]

### Methyl (6-(((6-((5,6,7,8-tetrahydroimidazo[1,2-a]pyridin-7-yl)methoxy)pyridin-3-yl)methyl)amino)isoquinolin-1-yl)carbamate

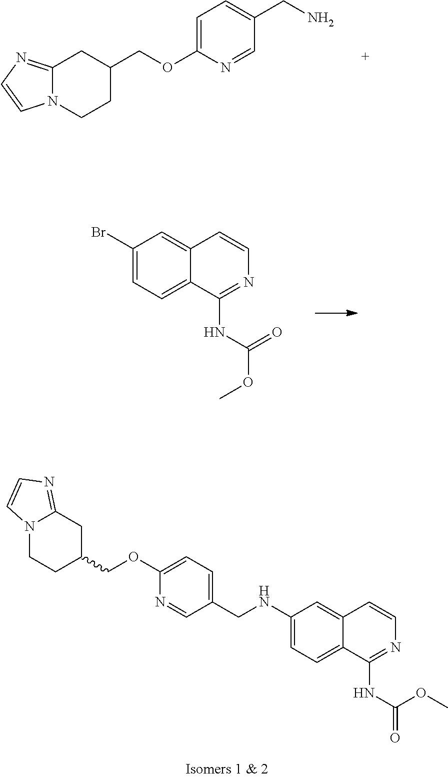 [SMILES: NCc1ccc(OCC2CCn3ccnc3C2)nc1 ; COC(=O)Nc1nccc2cc(Br)ccc12 ; COC(=O)Nc1nccc2cc(NCc3ccc(OCC4CCn5ccnc5C4)nc3)ccc12]

Following General Method 4, (6-((5,6,7,8-tetrahydroimidazo[1,2-a]pyridin-7-yl)methoxy)pyridin-3-yl)methanamine (108 mg, 0.42 mmol) was reacted with methyl (6-bromoisoquinolin-1-yl)carbamate (118 mg, 0.418 mmol) and NaOtBu (80 mg, 0.83 mmol) in THF (8 mL) at 60° C. for 1 h. After quenching the reaction mixture, the crude product was purified by flash chromatography (Silica, 0-20% (0.7M NH₃ in MeOH) in DCM). The two enantiomers were chirally separated by reverse phase chiral Gilson prep with UV detection at 260 nm, ambient column temp, a ChiralPAK IC 20×250 mm, 5 um Column flow rate 15 mL/min using 70% of MeCN with 30% of 0.1% Ammonia in water to yield: Enantiomer 1:

Methyl (R*)-(6-(((6-((5,6,7,8-tetrahydroimidazo[1,2-a]pyridin-7-yl)methoxy)pyridin-3-yl)methyl)amino)isoquinolin-1-yl)carbamate (61 mg, 0.13 mmol, 26%, 99% Purity), at 1.03 min, 99% purity (diode array).

The product was analysed by analytical RP Chiral HPLC (Agilent 1100 HPLC, ChiralPAK IC 2.1×150, 3 um column flow rate 0.4 mL/min eluting with 70/30 MeCN/0.1% Ammonia in Water; at 5.9 min, 100% purity (UV@240 nm)

[M+H]⁺=459.4

[M−H]⁻=457.3

### Enantiomer 2

Methyl (S*)-(6-(((6-((5,6,7,8-tetrahydroimidazo[1,2-a]pyridin-7-yl)methoxy)pyridin-3-yl)methyl)amino)isoquinolin-1-yl)carbamate (61 mg, 0.13 mmol, 25%, 97% Purity)

[M+H]⁺=459.4

[M−H]⁻=457.3, at 1.03 min, 97% purity (diode array).

The product was analysed by analytical RP Chiral HPLC (Agilent 1100 HPLC, ChiralPAK IC 2.1×150, 3 um column flow rate 0.4 mL/min eluting with 70/30 MeCN/0.1% Ammonia in Water; at 7.5 min, 100% purity (UV@240 nm).

### 6-N-({6-[(7R*)-5H,6H,7H,8H-imidazo[1,2-a]pyridin-7-ylmethoxy]pyridin-3-yl}methyl)isoquinoline-1,6-diamine (enantiomer 1, example number 1023)

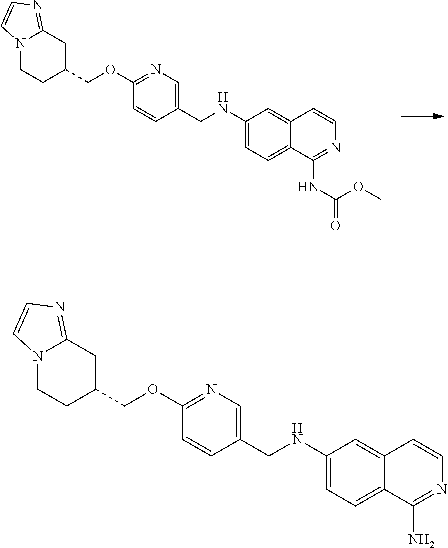

Enantiomer 1, methyl (R*)-(6-(((6-((5,6,7,8-tetrahydroimidazo[1,2-a]pyridin-7-yl)methoxy)pyridin-3-yl)methyl)amino)isoquinolin-1-yl)carbamate (60 mg, 0.13 mmol was deprotected using General Method 14a for 18 h. Following quenching, elution through an SCX and lyophilisation the product was isolated (24 mg, 45% yield) as a white solid.

[M+H]⁺=401.2

¹H NMR (500 MHz, DMSO-d6) δ 1.66-1.77 (1H, m), 2.09-2.16 (1H, m), 2.33-2.41 (1H, m), 2.45-2.50 (1H, m), 2.92 (1H, dd, J=16.4, 5.0 Hz), 3.88 (1H, td, J=12.0, 4.7 Hz), 4.04-4.11 (1H, m), 4.25 (2H, d, J=6.6 Hz), 4.30 (2H, d, J=5.7 Hz), 6.35 (2H, s), 6.54 (1H, d, J=2.3 Hz), 6.58 (1H, d, J=5.9 Hz), 6.70 (1H, t, J=5.8 Hz), 6.80 (1H, s), 6.84 (1H, d, J=8.5 Hz), 6.87 (1H, dd, J=9.0, 2.3 Hz), 6.99 (1H, s), 7.55 (1H, d, J=5.8 Hz), 7.73 (1H, dd, J=8.5, 2.5 Hz), 7.86 (1H, d, J=9.0 Hz), 8.20 (1H, d, J=2.4 Hz).

### 6-N-({6-[(7S*)-5H,6H,7H,8H-imidazo[1,2-a]pyridin-4-yl}-ylmethyl)isoquinoline-1,6-diamine (Enantiomer 2, Example Number 1024)

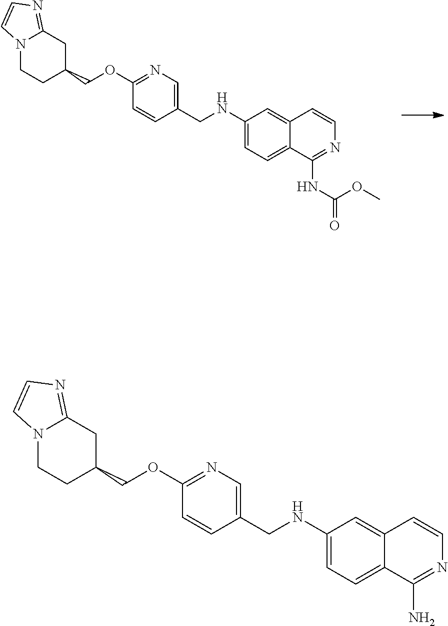 [SMILES: COC(=O)Nc1nccc2cc(NCc3ccc(OC[C@H]4CCn5ccnc5C4)nc3)ccc12 ; Nc1nccc2cc(NCc3ccc(OC[C@H]4CCn5ccnc5C4)nc3)ccc12]

Enantiomer 2, methyl (S*)-(6-(((6-((5,6,7,8-tetrahydroimidazo[1,2-a]pyridin-7-yl)methoxy)pyridin-3-yl)methyl)amino)isoquinolin-1-yl)carbamate (60 mg, 0.13 mmol) was deprotected using General Method 14a for 18 h. Following quenching, elution through an SCX and lyophilisation the product was isolated (29 mg, 54% yield) as an off white solid.

[M+H]⁺=401.2

¹H NMR (500 MHz, DMSO-d6) δ 1.66-1.77 (1H, m), 2.09-2.16 (1H, m), 2.32-2.42 (1H, m), 2.45-2.49 (1H, m), 2.92 (1H, dd, J=16.2, 5.0 Hz), 3.88 (1H, td, J=11.9, 4.7 Hz), 4.03-4.11 (1H, m), 4.25 (2H, d, J=6.6 Hz), 4.30 (2H, d, J=5.7 Hz), 6.33 (2H, s), 6.54 (1H, d, J=2.3 Hz), 6.57 (1H, d, J=5.9 Hz), 6.69 (1H, t, J=5.8 Hz), 6.80 (1H, d, J=1.2 Hz), 6.84 (1H, d, J=8.5 Hz), 6.87 (1H, dd, J=9.0, 2.3 Hz), 6.99 (1H, s), 7.55 (1H, d, J=5.8 Hz), 7.73 (1H, dd, J=8.5, 2.5 Hz), 7.85 (1H, d, J=9.0 Hz), 8.19 (1H, d, J=2.4 Hz)

### Example Number 2192

### N6-(2-chloro-4-(((1-methylpiperidin-4-yl)oxy)methyl)benzyl)isoquinoline-1,6-diamine

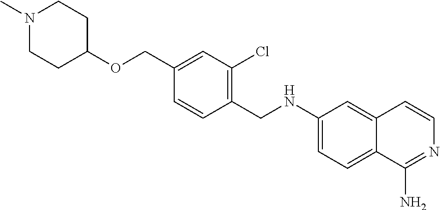 [SMILES: CN1CCC(OCc2ccc(CNc3ccc4c(N)nccc4c3)c(Cl)c2)CC1]

### Tert-butyl 4-((4-bromo-3-chlorobenzyl)oxy)piperidine-1-carboxylate

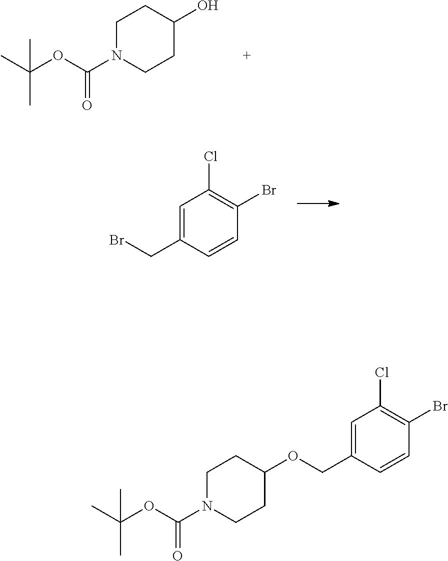 [SMILES: CC(C)(C)OC(=O)N1CCC(O)CC1 ; Clc1cc(CBr)ccc1Br ; CC(C)(C)OC(=O)N1CCC(OCc2ccc(Br)c(Cl)c2)CC1]

Using General Method 1a, tert-butyl 4-hydroxypiperidine-1-carboxylate (708 mg, 3.52 mmol) was reacted with 1-bromo-4-(bromomethyl)-2-chlorobenzene (1.00 g, 3.52 mmol). The crude product was purified by flash chromatography (Silica, 0-100% EtOAc in isohexane) to afford the product (990 mg, 68% yield) as a colourless solid.

[M-tBu+H]⁺=347.8

¹H NMR (500 MHz, DMSO-d6) δ 1.35-1.44 (11H, m), 1.79-1.86 (2H, m), 2.98-3.09 (2H, m), 3.56 (1H, tt, J=8.1, 3.7 Hz), 3.59-3.67 (2H, m), 4.51 (2H, s), 7.25 (1H, dd, J=8.2, 2.0 Hz), 7.57 (1H, d, J=2.0 Hz), 7.74 (1H, d, J=8.2 Hz).

### 4-((4-Bromo-3-chlorobenzyl)oxy)-1-methylpiperidine

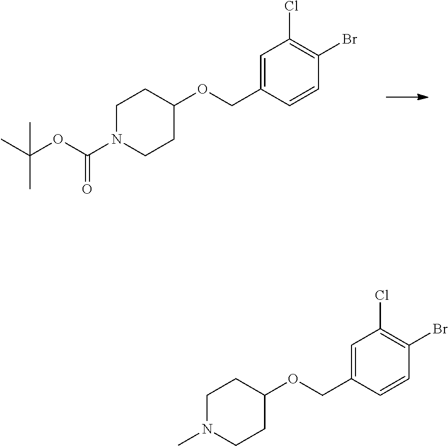 [SMILES: CC(C)(C)OC(=O)N1CCC(OCc2ccc(Br)c(Cl)c2)CC1 ; CN1CCC(OCc2ccc(Br)c(Cl)c2)CC1]

Tert-butyl 4-((4-bromo-3-chlorobenzyl)oxy)piperidine-1-carboxylate (990 mg, 2.45 mmol) was reacted using General Method 10 for 2 h. The reaction mixture was concentrated then taken up in EtOAc (50 mL), washed with 2M Na₂CO₃ (50 mL) and brine (30 mL). The organic phases were dried (MgSO₄), filtered and concentrated to afford the product (780 mg, 95% yield) as colourless oil.

[M+H]⁺=318.0

¹H NMR (500 MHz, DMSO-d6) δ 1.46-1.58 (2H, m), 1.80-1.88 (2H, m), 1.96-2.05 (2H, m), 2.14 (3H, s), 2.55-2.62 (2H, m), 3.36(1H, tt, J=8.5, 4.0 Hz), 4.48 (2H, s), 7.24 (1H, dd, J=8.2, 2.0 Hz), 7.56 (1H, d, J=2.0 Hz), 7.74 (1H, d, J=8.2 Hz).

### 2-Chloro-4-(((1-methylpiperidin-4-yl)oxy)methyl)benzonitrile

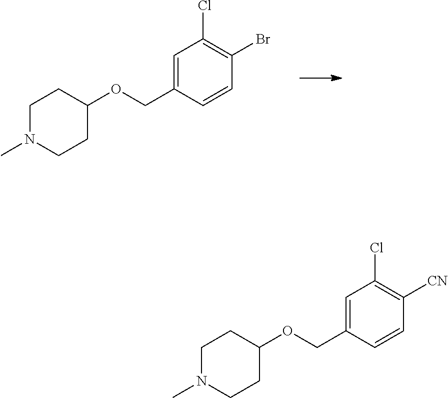 [SMILES: CN1CCC(OCc2ccc(Br)c(Cl)c2)CC1 ; CN1CCC(OCc2ccc(C#N)c(Cl)c2)CC1]

Using General Method 2, 4-((4-bromo-3-chlorobenzyl)oxy)-1-methylpiperidine (400 mg, 1.26 mmol) was reacted at 80° C. 16 h. concentrated. The crude product was purified by flash chromatography (Silica, 0-10% (0.7M NH₃ in MeOH) in DCM) to afford the product (203 mg, 83% yield) as a white solid.

[M+H]⁺=256.1

¹H NMR (500 MHz, DMSO-d6) δ 1.47-1.59 (2H, m), 1.82-1.91 (2H, m), 1.96-2.07 (2H, m), 2.14 (3H, s), 2.56-2.63 (2H, m), 3.39 (1H, tt, J=8.5, 4.1 Hz), 4.60 (2H, s), 7.48-7.51 (1H, m), 7.67 (1H, d, J=1.4 Hz), 7.96 (1H, d, J=8.0 Hz)

### (2-Chloro-4-(((1-methylpiperidin-4-yl)oxy)methyl)phenyl)methanamine

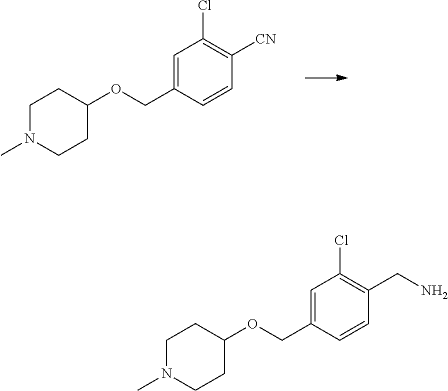 [SMILES: CN1CCC(OCc2ccc(C#N)c(Cl)c2)CC1 ; CN1CCC(OCc2ccc(CN)c(Cl)c2)CC1]

The nitrile, 2-chloro-4-(((1-methylpiperidin-4-yl)oxy)methyl)benzonitrile (185 mg, 0.70 mmol) was reduced following General Method 3b for 16 h. The product was isolated (162 mg, 82% yield) as a yellow gum.

[M+H]⁺=269.0

### Tert-butyl (6-((2-chloro-4-(((1-methylpiperidin-4-yl)oxy)methyl)benzyl)amino)isoquinolin-1-yl)carbamate

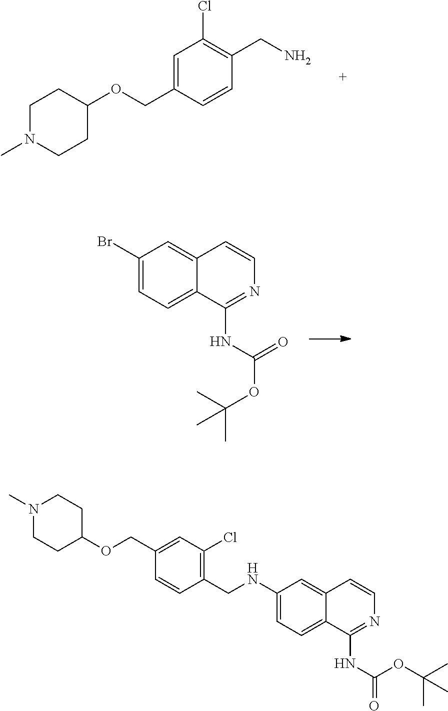 [SMILES: CN1CCC(OCc2ccc(CN)c(Cl)c2)CC1 ; CC(C)(C)OC(=O)Nc1nccc2cc(Br)ccc12 ; CN1CCC(OCc2ccc(CNc3ccc4c(NC(=O)OC(C)(C)C)nccc4c3)c(Cl)c2)CC1]

Using General Method 4, (2-chloro-4-(((1-methylpiperidin-4-yl)oxy)methyl)phenyl)methanamine (100 mg, 0.37 mmol) was reacted with tert-butyl (6-bromoisoquinolin-1-yl)carbamate (120 mg, 0.37 mmol) in the presence of 1M KO^{t}Bu in THF (0.74 mL, 0.74 mmol) in THF (4 mL) at 60° C. for 8 h. After quenching the reaction, the crude product was purified by flash chromatography (Silica, 0-10% (0.7M NH₃ in MeOH) in DCM) to afford the product (38 mg, 19% yield) as an off-white solid.

[M+H]⁺=511.2

### N6-(2-chloro-4-(((1-methylpiperidin-4-yl)oxy)methyl)benzyl)isoquinoline-1,6-diamine

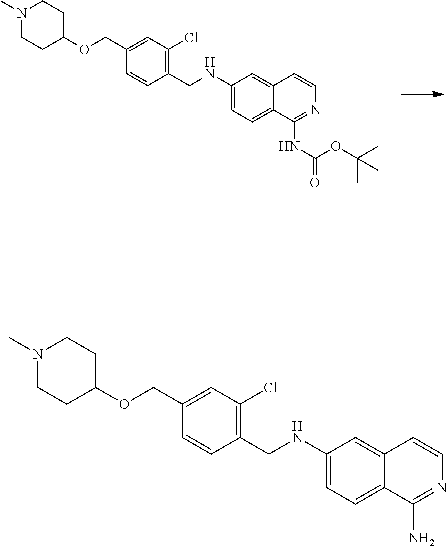

Tert-butyl (6-((2-chloro-4-(((1-methylpiperidin-4-yl)oxy)methyl)benzyl)amino)isoquinolin-1-yl)carbamate (38 mg, 0.074 mmol) was deprotected using General Method 7b. After elution through an SCX, the crude product was purified by flash chromatography (Silica, 0-20% (0.7M NH₃ in MeOH) in DCM). The product was lyophilised to afford the product (12 mg, 38% yield) as a white solid.

[M+H]⁺=411.2

¹H NMR (500 MHz, DMSO-d6) δ 1.44-1.53 (2H, m), 1.79-1.86 (2H, m), 1.95-2.02 (2H, m), 2.12 (3H, s), 2.54-2.61 (2H, m), 3.32-3.38 (1H, m), 4.41 (2H, d, J=5.9 Hz), 4.47 (2H, s), 6.30 (2H, s), 6.41 (1H, d, J=2.3 Hz), 6.54 (1H, d, J=5.8 Hz), 6.78 (1H, t, J=6.0 Hz), 6.89 (1H, dd, J=9.0, 2.4 Hz), 7.23 (1H, dd, J=8.0, 1.7 Hz), 7.38 (1H, d, J=7.9 Hz), 7.42 (1H, d, J=1.6 Hz), 7.54 (1H, d, J=5.9 Hz), 7.87 (1H, d, J=9.0 Hz).

### Example Number 2177

### N6-(4-(2-(1-methylpiperidin-4-yl)ethyl)benzyl)isoquinoline-1,6-diamine

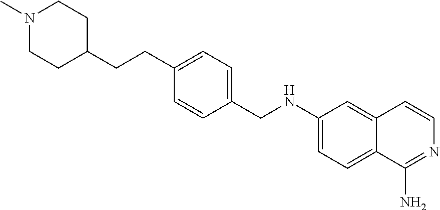 [SMILES: CN1CCC(CCc2ccc(CNc3ccc4c(N)nccc4c3)cc2)CC1]

### Tert-butyl 4-((4-cyanophenyl)ethynyl)piperidine-1-carboxylate

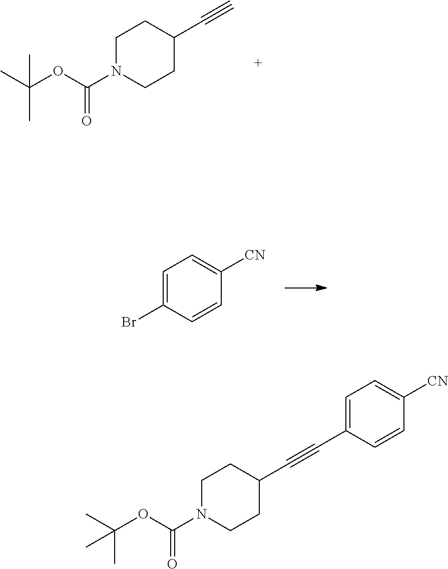 [SMILES: C#CC1CCN(C(=O)OC(C)(C)C)CC1 ; N#Cc1ccc(Br)cc1 ; CC(C)(C)OC(=O)N1CCC(C#Cc2ccc(C#N)cc2)CC1]

A suspension of 4-bromobenzonitrile (1.04 g, 5.73 mmol), tert-butyl 4-ethynylpiperidine-1-carboxylate (1.00 g, 4.78 mmol) and copper (1) iodide (46 mg, 0.24 mmol) in NEt₃ (10 mL) was purged with N₂ before Pd(PPh₃)₄(552 mg, 0.48 mmol) was added and the mixture was purged for a further 30 min with N₂. The reaction was heated to 90° C. and stirred for 16 h. The reaction was allowed to cool, and water was added (30 mL) before extracting the aqueous layer with EtOAc (3×30 mLQ. The combined organics were dried (MgSO₄), filtered and concentrated. The crude product was purified by flash chromatography (Silica, 0-100% EtOAc in Isohexane) to afford the product (1.59 g, 96% yield) as an orange solid.

[M-tBu+H]⁺=255.1

¹H NMR (500 MHz, DMSO-d6) δ 1.40 (9H, s), 1.48-1.57 (2H, m), 1.78-1.86 (2H, m), 2.87-2.94 (1H, m), 3.09-3.20 (2H, m), 3.61-3.67 (2H, m), 7.55-7.61 (2H, m), 7.79-7.85 (2H, m) ppm.

### 4-((1-Methylpiperidin-4-yl)ethynyl)benzonitrile

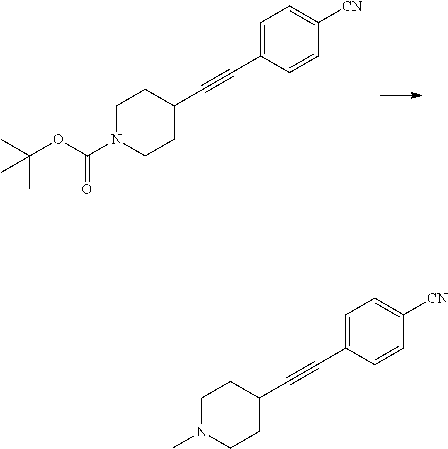 [SMILES: CC(C)(C)OC(=O)N1CCC(C#Cc2ccc(C#N)cc2)CC1 ; CN1CCC(C#Cc2ccc(C#N)cc2)CC1]

Following General Method 10, tert-butyl 4-((4-cyanophenyl)ethynyl)piperidine-1-carboxylate (1.00 g, 3.22 mmol) was reacted for 2 h. The reaction mixture was concentrated then taken up in EtOAc (50 mL), washed with 2M Na₂CO₃ (50 mL) and brine (3×30 mL). The organic phases were dried (MgSO₄), filtered, and concentrated to afford the product (451 mg, 59% yield) as an off-white solid.

[M+H]⁺=225.1

¹H NMR (500 MHz, DMSO-d6) δ 1.57-1.70 (2H, m), 1.82-1.91 (2H, m), 2.12-2.25 (5H, m), 2.60-2.72 (3H, m), 7.54-7.59 (2H, m), 7.80-7.84 (2H, m) ppm.

### 4-(2-(1-Methylpiperidin-4-yl)ethyl)benzonitrile

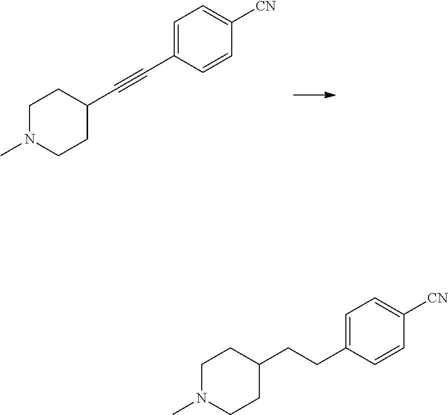 [SMILES: CN1CCC(C#Cc2ccc(C#N)cc2)CC1 ; CN1CCC(CCc2ccc(C#N)cc2)CC1]

To a solution of 4-((1-methylpiperidin-4-yl)ethynyl)benzonitrile (100 mg, 0.45 mmol) in EtOH (5 mL) was added 10% Pd/C (50 mg, 0.05 mmol) and was stirred under H₂ (3 bar) in a steel-autoclave for 16 h. The crude reaction was filtered through Celite® and washed with EtOH (10 mL) before concentrating in vacuo to obtain the product (99 mg, 92% yield) as a white solid.

[M+H]⁺=229.2

¹H NMR (500 MHz, DMSO-d6) δ 1.12-1.20 (3H, m), 1.46-1.53 (2H, m), 1.60-1.69 (2H, m), 1.76-1.82 (2H, m), 2.13 (3H, s), 2.64-2.70 (2H, m), 2.71-2.77 (2H, m), 7.40-7.44 (2H, m), 7.72-7.75 (2H, m) ppm.

### (4-(2-(1-Methyl piperidin-4-yl)ethyl)phenyl)methanamine

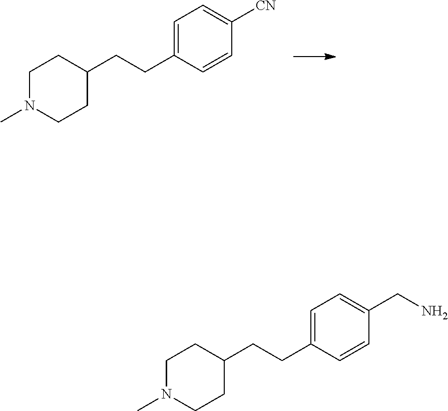 [SMILES: CN1CCC(CCc2ccc(C#N)cc2)CC1 ; CN1CCC(CCc2ccc(CN)cc2)CC1]

The nitrile, 4-(2-(1-methylpiperidin-4-yl)ethyl)benzonitrile (165 mg, 0.72 mmol) was reduced according to General Method 3b for 16 h. The product was isolated (175 mg, 89% yield) as a yellow gum and used without further purification.

[M+H]⁺=233.2

¹H NMR (500 MHz, DMSO-d6) δ 1.10-1.19 (3H, m), 1.44-1.51 (2H, m), 1.60-1.67 (2H, m), 1.73-1.79 (2H, m), 1.85-2.04 (2H, m), 2.11 (3H, s), 2.53-2.58 (2H, m), 2.68-2.74 (2H, m), 3.66 (2H, s), 7.08-7.12 (2H, m), 7.19-7.23 (2H, m) ppm.

### Tert-butyl (6-((4-(2-(1-methylpiperidin-4-yl)ethyl)benzyl)amino)isoquinolin-1-yl)carbamate

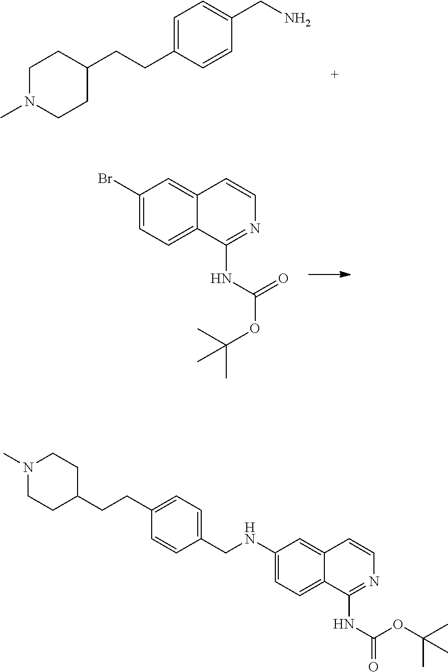 [SMILES: CN1CCC(CCc2ccc(CN)cc2)CC1 ; CC(C)(C)OC(=O)Nc1nccc2cc(Br)ccc12 ; CN1CCC(CCc2ccc(CNc3ccc4c(NC(=O)OC(C)(C)C)nccc4c3)cc2)CC1]

Following General Method 4, (4-(2-(1-methylpiperidin-4-yl)ethyl)phenyl)methanamine (85 mg, 0.37 mmol) was reacted with tert-butyl (6-bromoisoquinolin-1-yl)carbamate (124 mg, 0.38 mmol) in the presence of 2M KO^{t}Bu in THF (0.37 mL, 0.73 mmol) in THF (4 mL) at 60° C. for 1 h. After elution through an SCX, the crude product was purified by flash chromatography (Silica, 0-20% (0.7M NH₃ in MeOH) in DCM) to afford the product (62 mg, 32% yield).

[M+H]1=475.3

### N6-(4-(2-(1-methylpiperidin-4-yl)ethyl)benzyl)isoquinoline-1,6-diamine dihydrochloride

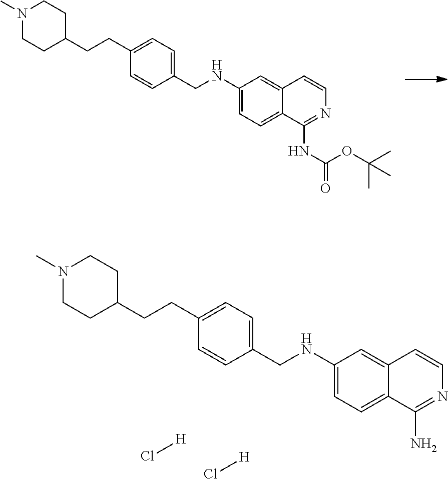

Tert-butyl (6-((4-(2-(1-methylpiperidin-4-yl)ethyl)benzyl)amino)isoquinolin-1-yl)carbamate (62 mg, 0.13 mmol) was deprotected following General Method 7b. The crude product was purified by flash chromatography (Silica, 0-20% (0.7M NH₃ in MeOH) in DCM) to afford the product (35 mg, 57% yield) as a off-white solid.

[M+H]⁺=375.3

¹H NMR (500 MHz, DMSO-d6) δ 1.35-1.45 (3H, m), 1.45-1.53 (2H, m), 1.78-1.87 (2H, m), 2.55-2.61 (5H, m), 2.61-2.70 (2H, m), 3.15-3.22 (2H, m), 4.38 (2H, d, J=5.9 Hz), 6.67 (1H, d, J=2.3 Hz), 6.74 (1H, d, J=6.8 Hz), 7.03 (1H, dd, J=9.2, 2.3 Hz), 7.18 (2H, m), 7.30 (2H, m), 7.44-7.49 (2H, m), 7.88 (2H, s), 8.11 (1H, d, J=9.2 Hz), 11.34 (1H, s) ppm. 1×exchangeable proton not observed.

### Example Number 2193

### N-(4-(((1-methylpiperidin-4-yl)oxy)methyl)benzyl)isoquinolin-6-amine

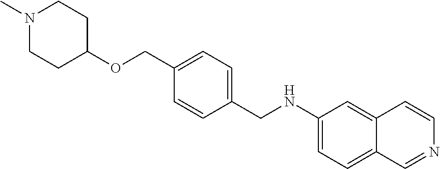 [SMILES: CN1CCC(OCc2ccc(CNc3ccc4cnccc4c3)cc2)CC1]

### N-(4-(((1-methylpiperidin-4-yl)oxy)methyl)benzyl)isoquinolin-6-amine

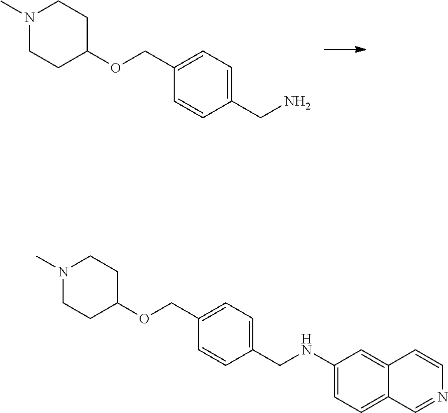 [SMILES: CN1CCC(OCc2ccc(CN)cc2)CC1 ; CN1CCC(OCc2ccc(CNc3ccc4cnccc4c3)cc2)CC1]

Following General Method 4, (4-(((1-methylpiperidin-4-yl)oxy)methyl)phenyl)methanamine (50 mg, 0.21 mmol) was reacted with 6-bromoisoquinoline (44 mg, 0.21 mmol) in the presence of NaOtBu (41 mg, 0.43 mmol) in 1,4-dioxane (5 mL) at 90° C. for 18 h. After quenching the reaction mixture, the crude product was purified by prep HPLC and lyophilised (Waters, Basic (0.1% Ammonium Bicarbonate), Basic, Waters X-Bridge Prep-C18, 5 μm, 19×50 mm column, 10-40% MeCN in Water) to afford the product (14 mg, 18% yield) as a colourless solid.

[M+H]⁺=362.5

¹H NMR (500 MHz, DMSO-d6) δ 1.40-1.57 (2H, m), 1.77-1.87 (2H, m), 1.91-2.05 (2H, m), 2.12 (3H, s), 2.54-2.62 (2H, m), 3.34-3.37 (1H, m), 4.39 (2H, d, J=5.9 Hz), 4.46 (2H, s), 6.61 (1H, d, J=2.2 Hz), 7.11 (1H, t, J=5.9 Hz), 7.14 (1H, dd, J=8.9, 2.3 Hz), 7.26-7.32 (2H, m), 7.34 (1H, d, J=5.8 Hz), 7.35-7.40 (2H, m), 7.75 (1H, d, J=8.9 Hz), 8.15 (1H, d, J=5.8 Hz), 8.85 (1H, s) ppm.

### Example Number 2194

### N6-(4-(((3,3-difluoro-1-methylpiperidin-4-yl)oxy)methyl)benzyl)isoquinoline-1,6-diamine

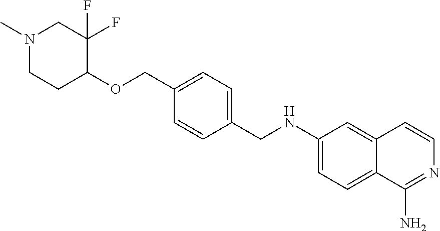 [SMILES: CN1CCC(OCc2ccc(CNc3ccc4c(N)nccc4c3)cc2)C(F)(F)C1]

### Tert-butyl 4-((4-cyanobenzyl)oxy)-3,3-difluoropiperidine-1-carboxylate

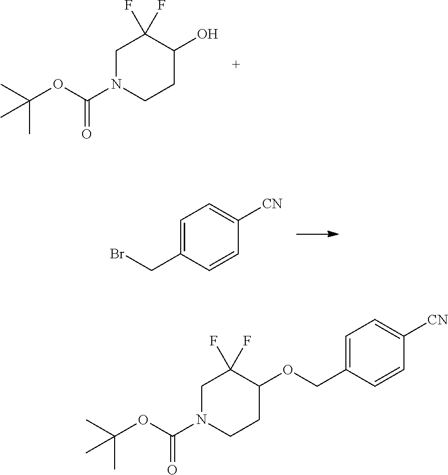 [SMILES: CC(C)(C)OC(=O)N1CCC(O)C(F)(F)C1 ; N#Cc1ccc(CBr)cc1 ; CC(C)(C)OC(=O)N1CCC(OCc2ccc(C#N)cc2)C(F)(F)C1]

Using General Method 1a, tert-butyl 3,3-difluoro-4-hydroxypiperidine-1-carboxylate (500 mg, 2.11 mmol) was reacted with 4-(bromomethyl)benzonitrile (413 mg, 2.11 mmol). The crude product was purified by flash chromatography (Silica, 0-50% EtOAc in isohexane) to afford the product (490 mg, 63% yield) as a thick colourless oil.

[M-boc+H]⁺=253.3

¹H NMR (500 MHz, DMSO-d6) 1.40 (9H, s), 1.67-1.78 (1H, m), 1.85-1.95 (1H, m), 3.47-3.56 (1H, m), 3.55-3.65 (1H, m), 3.73-3.86 (1H, m), 3.88-4.00 (1H, m), 4.70-4.85 (2H, m), 7.50-7.59 (2H, m), 7.80-7.87 (2H, m). CH₂ obscured by water.

### 4-(((3,3-difluoro-1-methylpiperidin-4-yl)oxy)methyl)benzonitrile

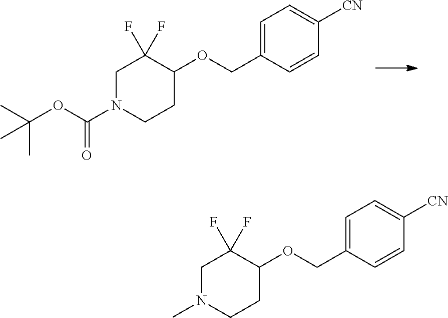 [SMILES: CC(C)(C)OC(=O)N1CCC(OCc2ccc(C#N)cc2)C(F)(F)C1 ; CN1CCC(OCc2ccc(C#N)cc2)C(F)(F)C1]

Following General Method 10, tert-butyl 4-((4-cyanobenzyl)oxy)-3,3-difluoropiperidine-1-carboxylate (400 mg, 1.14 mmol) was reacted for 18 h. The crude product was purified by flash chromatography (Silica, 0-10% (0.7M NH₃ in MeOH) in DCM) to afford the product (211 mg, 67% yield) as a thick colourless oil.

[M+H]⁺=267.3

¹H NMR (500 MHz, DMSO-d6) 1.69-1.79 (1H, m), 1.86-1.96 (1H, m), 2.22 (3H, s), 2.24 (1H, s), 2.73-2.84 (1H, m), 3.71-3.81 (1H, m), 4.69-4.83 (2H, m), 7.50-7.58 (2H, m), 7.80-7.87 (2H, m). CH₂ obscured by DMSO.

### (4-(((3,3-difluoro-1-methylpiperidin-4-yl)oxy)methyl)phenyl)methanamine

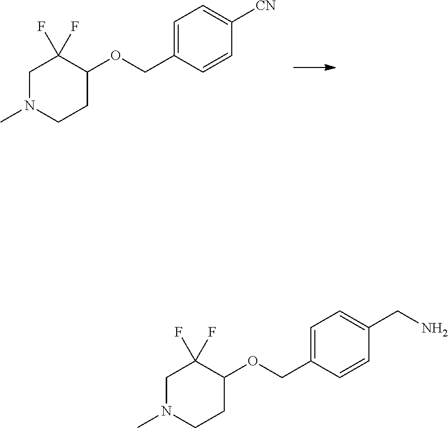 [SMILES: CN1CCC(OCc2ccc(C#N)cc2)C(F)(F)C1 ; CN1CCC(OCc2ccc(CN)cc2)C(F)(F)C1]

The nitrile, 4-(((3,3-difluoro-1-methylpiperidin-4-yl)oxy)methyl)benzonitrile (200 mg, 0.75 mmol) was reduced following General Method 3b. The product was isolated (200 mg, 94% yield) as a colourless solid and used without further purification.

[M+H]⁺=271.4

¹H NMR (500 MHz, DMSO-d6) δ 1.31-1.39 (2H, m), 1.67-1.89 (4H, m), 2.20 (3H, s), 2.22-2.26 (1H, m), 2.67-2.80 (1H, m), 3.60-3.66 (1H, m), 3.70 (2H, s), 4.61 (2H, s), 7.25-7.29 (2H, m), 7.29-7.33 (2H, m).

### Tert-butyl (6-((4-(((3,3-difluoro-1-methylpiperidin-4-yl)oxy)methyl)benzyl)amino)isoquinolin-1-yl)carbamate

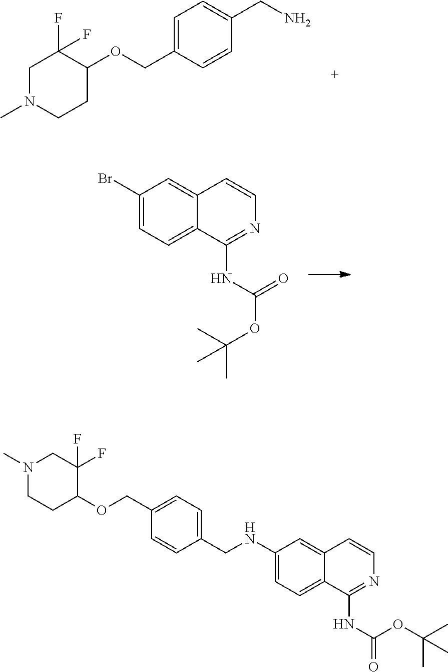 [SMILES: CN1CCC(OCc2ccc(CN)cc2)C(F)(F)C1 ; CC(C)(C)OC(=O)Nc1nccc2cc(Br)ccc12 ; CN1CCC(OCc2ccc(CNc3ccc4c(NC(=O)OC(C)(C)C)nccc4c3)cc2)C(F)(F)C1]

Using General Method 4, (4-(((3,3-difluoro-1-methylpiperidin-4-yl)oxy)methyl)phenyl)methanamine (197 mg, 0.73 mmol) was reacted with tert-butyl(6-bromoisoquinolin-1-yl)carbamate (236 mg, 0.73 mmol) and NaOtBu (140 mg, 1.46 mmol) in THF (3 mL) at 60° C. for 1 h. After quenching the reaction mixture, the crude product was purified by flash chromatography (Silica, 0-20% (0.7M NH₃ in MeOH) in DCM) to afford the product (212 mg, 54% yield) as a cream solid

[M+H]⁺=513.5

### N6-(4-(((3,3-difluoro-1-methylpiperidin-4-yl)oxy)methyl)benzyl)isoquinoline-1,6-diamine

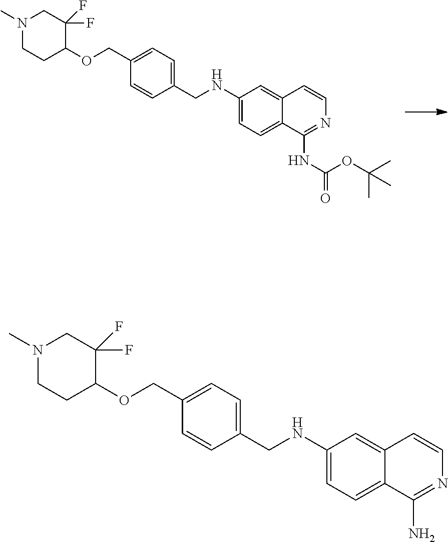

Tert-butyl (6-((4-(((3,3-difluoro-1-methylpiperidin-4-yl)oxy)methyl)benzyl)amino)isoquinolin-1-yl)carbamate (42 mg, 0.082 mmol) was deprotected using General Method 7b. The crude product was purified by flash chromatography (Silica, 0-20% (0.7 M NH₃ in MeOH) in DCM) and lyophilised to the product (26 mg, 75% yield) as a colourless solid.

[M+H]⁺=413.2

¹H NMR (500 MHz, DMSO-d6) δ 1.66-1.77 (1H, m), 1.80-1.89 (1H, m), 2.16-2.24 (4H, m), 2.42-2.51 (2H, m), 2.68-2.80 (1H, m), 3.62-3.71 (1H, m), 4.36 (2H, d, J=5.6 Hz), 4.61 (2H, s), 6.32 (2H, d, J=6.7 Hz), 6.47 (1H, d, J=2.3 Hz), 6.53 (1H, d, J=5.9 Hz), 6.78 (1H, t, J=6.0 Hz), 6.88 (1H, dd, J=9.0, 2.3 Hz), 7.27-7.32 (2H, m), 7.35-7.39 (2H, m), 7.53 (1H, d, J=5.8 Hz), 7.85 (1H, d, J=9.0 Hz), 2×C—H signals obscured by DMSO observed from COSY and HSQC.

### Example Number 1008

### N-((6-((1-methylpiperidin-4-yl)methoxy)pyridin-3-yl)methyl)isoquinolin-6-amine

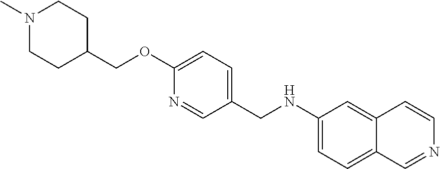 [SMILES: CN1CCC(COc2ccc(CNc3ccc4cnccc4c3)cn2)CC1]

### N-((6-((1-methylpiperidin-4-yl)methoxy)pyridin-3-yl)methyl)isoquinolin-6-amine

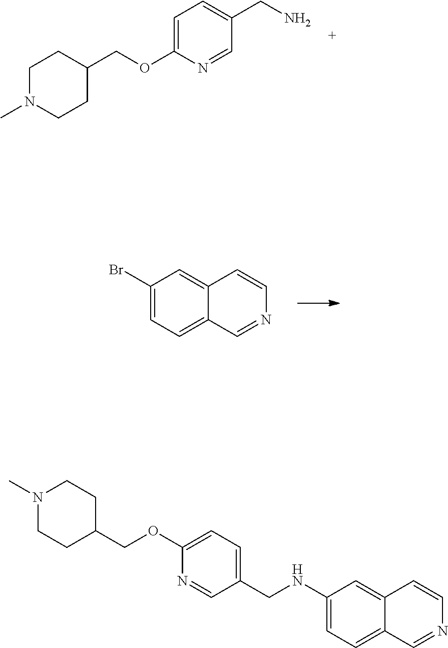 [SMILES: CN1CCC(COc2ccc(CN)cn2)CC1 ; Brc1ccc2cnccc2c1 ; CN1CCC(COc2ccc(CNc3ccc4cnccc4c3)cn2)CC1]

Following General Method 4, (6-((1-methylpiperidin-4-yl)methoxy)pyridin-3-yl)methanamine (40 mg, 0.17 mmol) was reacted with 6-bromoisoquinoline (40 mg, 0.19 mmol) and NaOtBu (35 mg, 0.36 mmol) in THF (4 mL) at 60° C. for 1 h. After quenching the reaction, the crude product was purified by flash chromatography (Silica, 0-10% (0.7M NH₃ in MeOH) in DCM) to afford the product (39 mg, 62% yield) as a colourless solid.

[M+H]⁺=363.2

¹H NMR (500 MHz, DMSO-d6) 1.22-1.31 (2H, m), 1.64-1.72 (3H, m), 1.81-1.90 (2H, m), 2.16 (3H, s), 2.74-2.81 (2H, m), 4.08 (2H, d, J=6.1 Hz), 4.33 (2H, d, J=5.6 Hz), 6.68 (1H, d, J=2.3 Hz), 6.79 (1H, d, J=8.5 Hz), 7.02 (1H, t, J=5.7 Hz), 7.11 (1H, dd, J=8.9, 2.3 Hz), 7.38 (1H, d, J=5.8 Hz), 7.72 (1H, dd, J=8.5, 2.5 Hz), 7.75 (1H, d, J=8.9 Hz), 8.18 (1H, d, J=5.8 Hz), 8.20 (1H, d, J=2.5 Hz), 8.86 (1H, s).

### Example Number 1009

### N5-((6-((1-methylpiperidin-4-yl)methoxy)pyridin-3-yl)methyl)isoquinoline-1,5-diamine

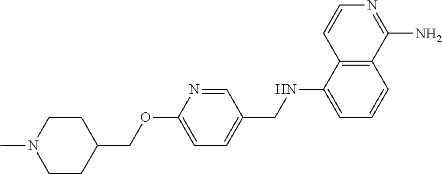 [SMILES: CN1CCC(COc2ccc(CNc3cccc4c(N)nccc34)cn2)CC1]

### Tert-butyl (5-(((6-((1-methylpiperidin-4-yl)methoxy)pyridin-3-yl)methyl)amino)isoquinolin-1-yl)carbamate

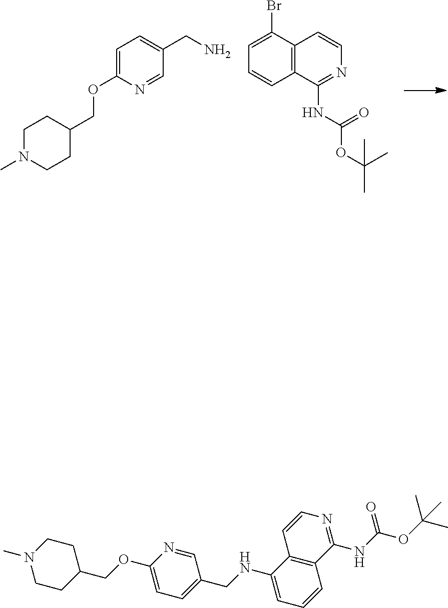 [SMILES: CN1CCC(COc2ccc(CN)cn2)CC1 ; CC(C)(C)OC(=O)Nc1nccc2c(Br)cccc12 ; CN1CCC(COc2ccc(CNc3cccc4c(NC(=O)OC(C)(C)C)nccc34)cn2)CC1]

Using General Method 4, (6-((1-methylpiperidin-4-yl)methoxy)pyridin-3-yl)methanamine (58 mg, 0.25 mmol) was reacted with tert-butyl (5-bromoisoquinolin-1-yl)carbamate (80 mg, 0.25 mmol) and NaOtBu (50 mg, 0.52 mmol) in THF (6 mL) at 60° C. for 1 h. After quenching the reaction mixture, the crude product was purified by flash chromatography (Silica, 0-20% (0.7 M NH₃ in MeOH) in DCM) to afford the product (59 mg, 49% yield) as a colourless solid.

[M+H]⁺=478.3

### N5-((6-((1-methylpiperidin-4-yl)methoxy)pyridin-3-yl)methyl)isoquinoline-1,5-diamine

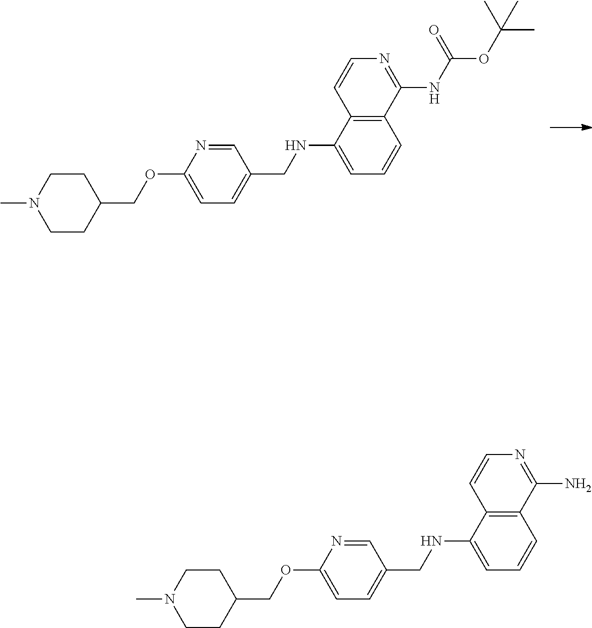

Tert-butyl (5-(((6-((1-methylpiperidin-4-yl)methoxy)pyridin-3-yl)methyl)amino)isoquinolin-1-yl)carbamate (59 mg, 0.12 mmol) was deprotected using General Method 7b. The crude product was purified by flash chromatography (Silica, 0-20% (0.7 M NH₃ in MeOH) in DCM) and lyophilised to afford the product (35 mg, 74% yield) as a colourless solid.

[M+H]⁺=378.2

¹H NMR (500 MHz, DMSO-d6) 1.21-1.32 (2H, m), 1.64-1.73 (3H, m), 1.81-1.94 (2H, m), 2.17 (3H, s), 2.74-2.82 (2H, m), 4.06 (2H, d, J=6.1 Hz), 4.38 (2H, d, J=5.7 Hz), 6.50 (2H, s), 6.54 (1H, d, J=7.7 Hz), 6.65 (1H, t, J=5.9 Hz), 6.74 (1H, d, J=8.5 Hz), 7.12-7.18 (2H, m), 7.32 (1H, d, J=8.3 Hz), 7.69 (1H, dd, J=8.5, 2.5 Hz), 7.74 (1H, d, J=6.1 Hz), 8.16 (1H, d, J=2.4 Hz)

### Example Number 1011

### 6-((4-(((1-Methylpiperidin-4-yl)oxy)methyl)benzyl)oxy)isoquinolin-1-amine

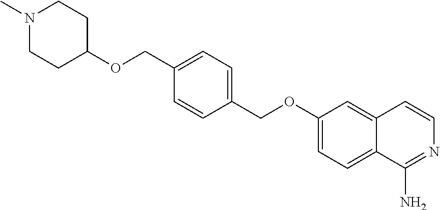 [SMILES: CN1CCC(OCc2ccc(COc3ccc4c(N)nccc4c3)cc2)CC1]

### Tert-butyl((4-(chloromethyl)benzyl)oxy)dimethylsilane

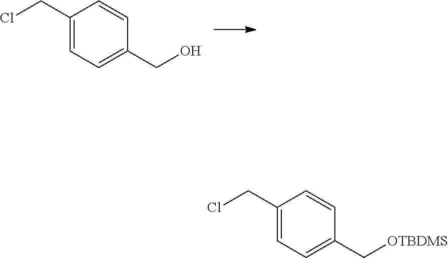 [SMILES: OCc1ccc(CCl)cc1 ; CC(C)(C)[Si](C)(C)OCc1ccc(CCl)cc1]

Tert-butylchlorodimethylsilane (529 mg, 3.51 mmol) was added to a solution of (4-(chloromethyl)phenyl)methanol (500 mg, 3.19 mmol) and imidazole (283 mg, 4.15 mmol) in DCM (5 mL) while cooling in an ice/water bath. The reaction was allowed to warm to rt and stirred for 1 h. The reaction was quenched with KHSO₄ (aq) (10 mL) and the layers separated. The organic layer was dried (Na₂SO₄), filtered and concentrated to obtain the product (861 mg, 95% yield) as a clear, colourless liquid, which was used without further purification.

¹H NMR (500 MHz, DMSO-d6) 0.08 (6H, s), 0.91 (9H, s), 4.72 (2H, s), 4.75 (2H, s), 7.29-7.33 (2H, m), 7.39-7.42 (2H, m).

### Tert-butyl 4-((4-(((tert-butyldimethylsilyl)oxy)methyl)benzyl)oxy)piperidine-1-carboxylate

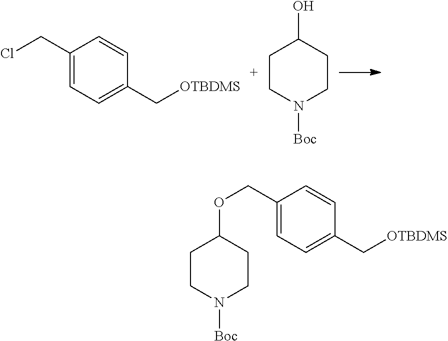 [SMILES: CC(C)(C)[Si](C)(C)OCc1ccc(CCl)cc1 ; CC(C)(C)OC(=O)N1CCC(O)CC1 ; CC(C)(C)OC(=O)N1CCC(OCc2ccc(CO[Si](C)(C)C(C)(C)C)cc2)CC1]

Following General Method 5a, tert-butyl 4-hydroxypiperidine-1-carboxylate (639 mg, 3.17 mmol) was reacted with tert-butyl((4-(chloromethyl)benzyl)oxy)dimethylsilane (860 mg, 3.17 mmol) for 20 h. The crude product was purified by flash chromatography (Silica, 0-100% EtOAc in isohexane) to afford the product (466 mg, 28% yield) as a clear colourless oil.

[M-boc+H]⁺=336.2

¹H NMR (500 MHz, DMSO-d6) 0.08 (6H, s), 0.90 (9H, s), 1.35-1.43 (11H, m), 1.77-1.86 (2H, m), 3.01-3.08 (2H, m), 3.51-3.57 (1H, m), 3.59-3.66 (2H, m), 4.50 (2H, s), 4.70 (2H, s), 7.26-7.31 (4H, m).

### 4-((4-(((Tert-butyldimethylsilyl)oxy)methyl)benzyl)oxy)-1-methylpiperidine

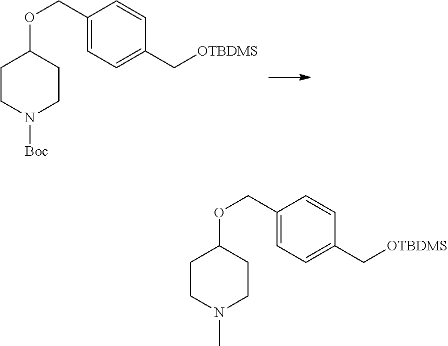 [SMILES: CC(C)(C)OC(=O)N1CCC(OCc2ccc(CO[Si](C)(C)C(C)(C)C)cc2)CC1 ; CN1CCC(OCc2ccc(CO[Si](C)(C)C(C)(C)C)cc2)CC1]

Using General Method 10, tert-butyl 4-((4-(((tert-butyldimethylsilyl)oxy)methyl)benzyl)oxy) piperidine-1-carboxylate (460 mg, 1.06 mmol) was reacted for 3 h. The reaction mixture was cooled to rt, treated with Na₂CO₃ (sat. aq., 30 mL) and extracted with EtOAc (3×20 mL). The organic phases were dried (MgSO₄), filtered and concentrated to afford the product (238 mg, 25% yield) as a yellow oil. The crude product was taken onto the next step without further purification.

[M+MeCN]1=392.2

### 4-(((1-methylpiperidin-4-yl)oxy)methyl)phenyl)methanol

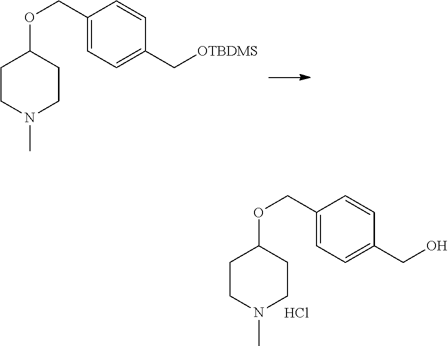 [SMILES: CN1CCC(OCc2ccc(CO[Si](C)(C)C(C)(C)C)cc2)CC1 ; CN1CCC(OCc2ccc(CO)cc2)CC1 ; Cl]

TBAF (1M in THF) (2 mL, 2 mmol) was added to a solution of 4-((4-(((tert-butyldimethylsilyl)oxy)methyl)benzyl)oxy)-1-methylpiperidine (238 mg, 0.68 mmol) in THF (5 mL) and stirred at rt for 18 h. The reaction was diluted with water (5 mL) and concentrated. The crude mixture was dissolved in 1:1 DCM/MeOH, filtered and concentrated. The product was purified by flash chromatography (Silica, 0-20% (0.7M NH₃ in MeOH) in DCM) to afford the product (108 mg, 55% yield) as a clear colourless oil.

[M+H]⁺=236.1

¹H NMR (500 MHz, MeOH-d4) 1.64-1.77 (2H, m), 1.89-2.02 (2H, m), 2.23-2.33 (5H, m), 2.71-2.80 (2H, m), 3.46-3.55 (1H, m), 4.55 (2H, s), 4.61 (2H, s), 7.34 (4H, s). 1×exchangeable proton.

### 6-((4-(((1-Methylpiperidin-4-yl)oxy)methyl)benzyl)oxy)isoquinolin-1-amine

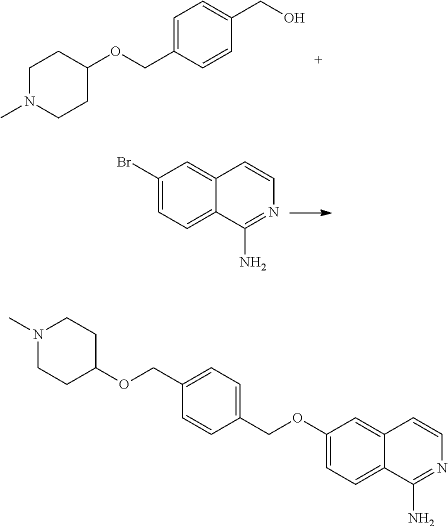 [SMILES: CN1CCC(OCc2ccc(CO)cc2)CC1 ; Nc1nccc2cc(Br)ccc12 ; CN1CCC(OCc2ccc(COc3ccc4c(N)nccc4c3)cc2)CC1]

Using General Method 1c, (4-(((1-methylpiperidin-4-yl)oxy)methyl)phenyl)methanol, (98 mg, 0.36 mmol) was reacted with 6-bromoisoquinolin-1-amine (80 mg, 0.36 mmol) for 4 h. The product was purified by prep HPLC (Mass directed 5-50% in basic mobile phase) and lyophilised (Waters, Basic (0.1% Ammonium Bicarbonate), Basic, Waters X-Bridge Prep-C18, 5 μm, 19×50 mm column, 5-50% MeCN in Water) to afford the product (4 mg, 3% yield) as an off-white solid.

[M+H]⁺=378.2

¹H NMR (500 MHz, DMSO-d6) 1.44-1.55 (2H, m), 1.81-1.89 (2H, m), 1.96-2.04 (2H, m), 2.13 (3H, s), 2.57-2.62 (2H, m), 3.33-3.38 (1H, m), 4.50 (2H, s), 5.21 (2H, s), 6.61 (2H, s), 6.80 (1H, d, J=5.8 Hz), 7.12 (1H, dd, J=9.1, 2.6 Hz), 7.19 (1H, d, J=2.6 Hz), 7.34-7.38 (2H, m), 7.44-7.49 (2H, m), 7.72 (1H, d, J=5.8 Hz), 8.10 (1H, d, J=9.1 Hz).

### Example Number 1012

### N6-((6-((1-isopropylpiperidin-4-yl)methoxy)pyridin-3-yl)methyl)isoquinoline-1,6-diamine

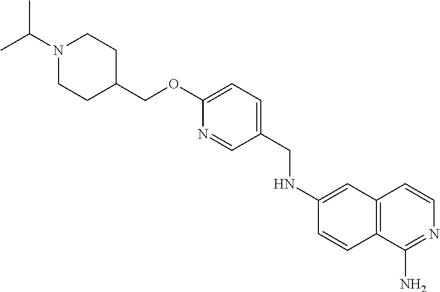 [SMILES: CC(C)N1CCC(COc2ccc(CNc3ccc4c(N)nccc4c3)cn2)CC1]

### 6-((1-Isopropylpiperidin-4-yl)methoxy)nicotinonitrile

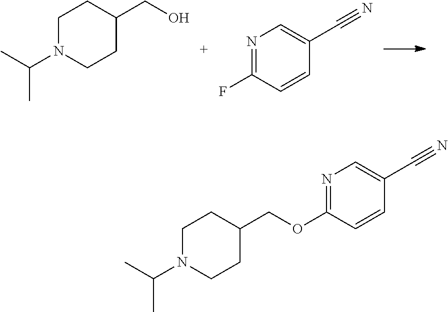 [SMILES: CC(C)N1CCC(CO)CC1 ; N#Cc1ccc(F)nc1 ; CC(C)N1CCC(COc2ccc(C#N)cn2)CC1]

Using General method 1b, (1-isopropylpiperidin-4-yl)methanol (300 mg, 1.91 mmol) was reacted with 6-fluoronicotinonitrile (233 mg, 1.91 mmol) at rt for 18 h. The reaction mixture was diluted with MeCN (20 mL) and filtered through a pad of Celite®. The filtrate was concentrated and the crude product was purified by flash chromatography (Silica, 0-10% (0.7M NH₃ in MeOH) in DCM) to afford the product (225 mg, 44% yield) as a yellow solid.

[M+H]⁺=260.3

¹H NMR (500 MHz, DMSO-d6) 0.96 (6H, d, J=6.9 Hz), 1.20-1.32 (2H, m), 1.65-1.78 (2H, m), 2.06-2.18 (2H, m), 2.62-2.73 (1H, m), 2.75-2.86 (2H, m), 3.26-3.31 (1H, m), 4.18 (2H, d, J=6.2 Hz), 7.00 (1H, d, J=8.7 Hz), 8.08-8.20 (1H, m), 8.62-8.72 (1H, m).

### (6-((1-Isopropylpiperidin-4-yl)methoxy)pyridin-3-yl)methanamine

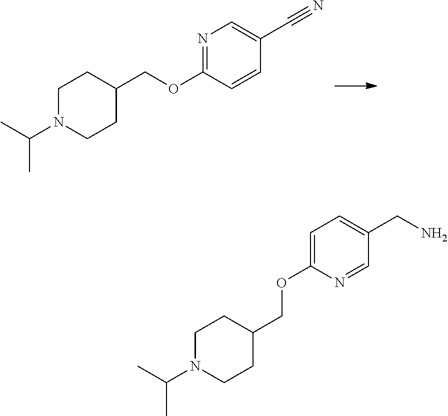 [SMILES: CC(C)N1CCC(COc2ccc(C#N)cn2)CC1 ; CC(C)N1CCC(COc2ccc(CN)cn2)CC1]

The nitrile, 6-((1-isopropylpiperidin-4-yl)methoxy)nicotinonitrile (215 mg, 0.83 mmol) was reduced according to General Method 3a using a Raney Ni cartridge for 90 min. The resultant solution was concentrated to afford the product (215 mg, 96% yield) as a colourless solid.

[M+H]1=264.4

¹H NMR (500 MHz, DMSO-d6) 0.95 (6H, d, J=6.6 Hz), 1.14-1.28 (2H, m), 1.68-1.74 (2H, m), 2.02-2.14 (2H, m), 2.62-2.71 (1H, m), 2.73-2.83 (2H, m), 2.81-3.05 (1H, m), 3.67 (2H, s), 4.06 (2H, d, J=6.1 Hz), 6.75 (1H, d, J=8.5 Hz), 7.67 (1H, dd, J=8.5, 2.5 Hz), 8.05 (1H, d, J=2.4 Hz). NH not observed

### Tert-butyl (6-(((6-((1-isopropylpiperidin-4-yl)methoxy)pyridin-3-yl)methyl)amino)isoquinolin-1-yl)carbamate

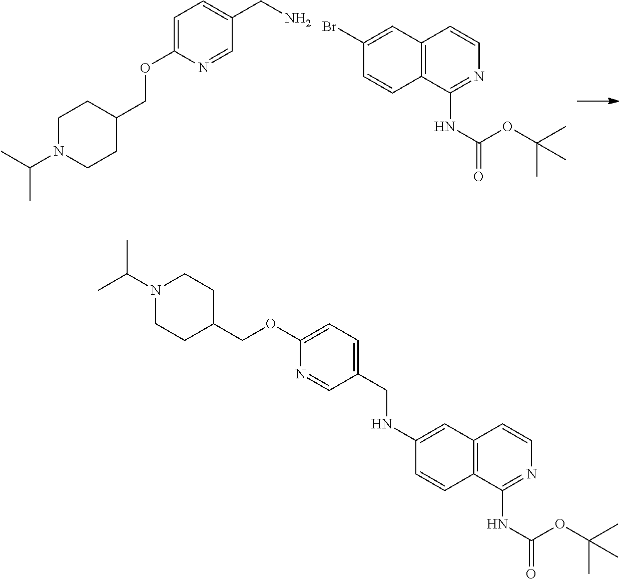

Using General Method 4, tert-butyl (6-bromoisoquinolin-1-yl)carbamate (123 mg, 0.38 mmol) was reacted with (6-((1-isopropylpiperidin-4-yl)methoxy)pyridin-3-yl)methanamine (100 mg, 0.38 mmol) and NaOtBu (73 mg, 0.76 mmol) in THF (6 mL) at 60° C. for 1 h. After quenching the reaction, the crude product was purified by flash chromatography (Silica, 0-20% (0.7M NH₃ in MeOH) in DCM) to afford the product (137 mg, 68% yield) as a cream solid.

[M+H]⁺=506.5

### N6-((6-((1-isopropylpiperidin-4-yl)methoxy)pyridin-3-yl)methyl)isoquinoline-1,6-diamine

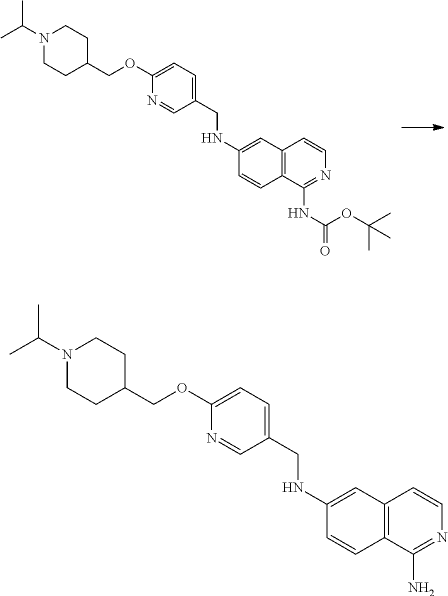

Tert-butyl (6-(((6-((1-isopropylpiperidin-4-yl)methoxy)pyridin-3-yl)methyl)amino)isoquinolin-1-yl)carbamate (133 mg, 0.26 mmol) was deprotected using General Method 7b. The crude product was purified by flash chromatography (Silica, 0-20% (0.7M NH₃ in MeOH) in DCM) to afford the product (63 mg, 58% yield) as a colourless solid.

[M+H]⁺=406.2

¹H NMR (500 MHz, DMSO-d6) 0.95 (6H, d, J=6.5 Hz), 1.15-1.28 (2H, m), 1.59-1.75 (3H, m), 2.02-2.13 (2H, m), 2.61-2.70 (1H, m), 2.72-2.81 (2H, m), 4.06 (2H, d, J=6.1 Hz), 4.28 (2H, d, J=5.4 Hz), 6.26-6.32 (2H, m), 6.53 (1H, d, J=2.3 Hz), 6.57 (1H, d, J=5.9 Hz), 6.67 (1H, t, J=5.9 Hz), 6.78 (1H, d, J=8.5 Hz), 6.84-6.88 (1H, m), 7.55 (1H, d, J=5.8 Hz), 7.66-7.73 (1H, m), 7.85 (1H, d, J=9.1 Hz), 8.17 (1H, d, J=2.4 Hz).

### Example Number 2210

### N-(1-aminoisoquinolin-6-yl)-4-(((1-methylpiperidin-4-yl)oxy)methyl)benzenesulfonamide

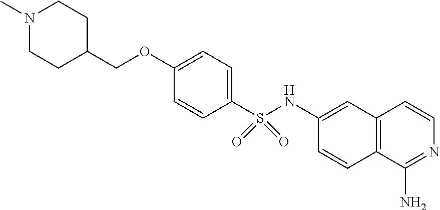 [SMILES: CN1CCC(COc2ccc(S(=O)(=O)Nc3ccc4c(N)nccc4c3)cc2)CC1]

### Tert-butyl 4-((4-bromobenzyl)oxy)piperidine-1-carboxylate

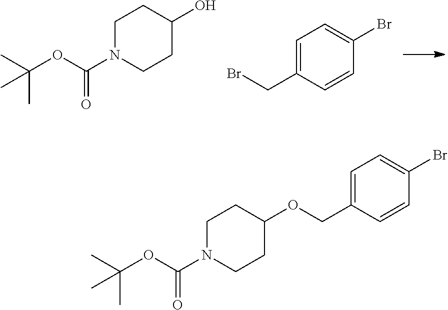 [SMILES: CC(C)(C)OC(=O)N1CCC(O)CC1 ; BrCc1ccc(Br)cc1 ; CC(C)(C)OC(=O)N1CCC(OCc2ccc(Br)cc2)CC1]

Following General Method 5a, tert-butyl 4-hydroxypiperidine-1-carboxylate (1.00 g, 4.97 mmol) was reacted with 1-bromo-4-(bromomethyl)benzene (1.24 g, 4.97 mmol) for 16 h. Sat. NaHCO₃ (30 mL) was added to the reaction mixture and the product was extracted into TBME (2×50 mL). The combined organic layers were dried (MgSO₄), filtered and concentrated. The crude product was purified by flash chromatography (Silica, 0-50% EtOAc in isohexane) to afford the product (1.44 g, 75% yield) as a colourless solid. [M-boc]⁺=270.2/271.9

¹H NMR (500 MHz DMSO-d 6) 1.34-1.45 (11H, m), 1.76-1.86 (2H, m), 2.98-3.10 (2H, m), 3.52-3.58 (1H, m), 3.58-3.66 (2H, m), 4.50 (2H, s), 7.27-7.32 (2H, m), 7.51-7.55 (2H, m).

### 4-((4-Bromobenzyl)oxy)-1-methylpiperidine

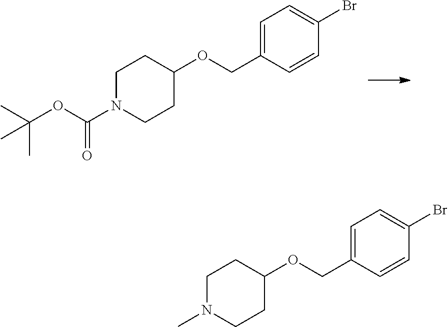 [SMILES: CC(C)(C)OC(=O)N1CCC(OCc2ccc(Br)cc2)CC1 ; CN1CCC(OCc2ccc(Br)cc2)CC1]

Using General Method 10, tert-butyl 4-((4-bromobenzyl)oxy)piperidine-1-carboxylate (1.85 g, 5.00 mmol) was reacted for 16 h. After elution through an SCX the product was isolated (1.31 g, 88% yield) as a clear orange liquid.

[M+H]⁺=284.2/286.2

¹H NMR (500 MHz, DMSO-d 6) 1.43-1.58 (2H, m), 1.76-1.88 (2H, m), 1.93-2.04 (2H, m), 2.13 (3H, s), 2.54-2.67 (2H, m), 3.33-3.39 (1H, m), 4.47 (2H, s), 7.22-7.32 (2H, m), 7.48-7.58 (2H, m)

### 4-(((1-Methylpiperidin-4-yl)oxy)methyl)benzenesulfonamide

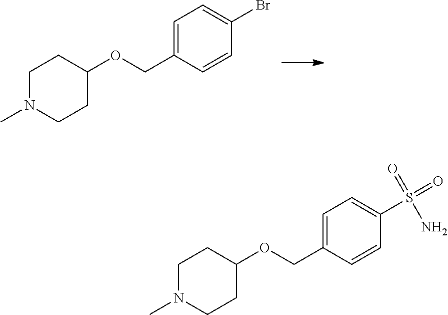 [SMILES: CN1CCC(OCc2ccc(Br)cc2)CC1 ; CN1CCC(OCc2ccc(S(N)(=O)=O)cc2)CC1]

A mixture of 4-((4-bromobenzyl)oxy)-1-methylpiperidine (1.30 g, 4.57 mmol) in THF (6 mL) was cooled in a dry ice/acetone bath and ⁺BuLi (2.5M in hexanes) (1.83 mL, 4.57 mmol) was added dropwise and the reaction stirred while continuing to cool in a dry ice/acetone bath for 1 h. Sulfuryl chloride (371 μL, 4.57 mmol) was added dropwise and the reaction mixture was stirred for 15 min in a dry ice/acetone bath. NH₃ (0.5M in 1,4-dioxane) (27 mL, 13.7 mmol) was added dropwise to the solution, which was then warmed to rt and stirred for 2 h. 1M HCl (aq.) (18 mL, 18.3 mmol) was added and the suspension was concentrated. The mixture was taken up into sat. K₂CO₃ (aq) (60 mL) and extracted into EtOAc (6×60 mL). The combined organic layers were dried (Na₂SO₄), filtered and concentrated. The crude product was purified by flash chromatography (Silica, 0-20% (0.7M NH₃ in MeOH) in DCM) to afford the product (272 mg, 20% yield) as a colourless solid.

[M+H]⁺=285.3

¹H NMR (500 MHz, DMSO-d 6) 1.46-1.57 (2H, m), 1.82-1.89 (2H, m), 1.96-2.04 (2H, m), 2.13 (3H, s), 2.54-2.63 (2H, m), 3.34-3.41 (1H, m), 4.57 (2H, s), 7.33 (2H, s), 7.43-7.57 (2H, m), 7.75-7.85 (2H, m).

### N-(1-aminoisoquinolin-6-yl)-4-(((1-methylpiperidin-4-yl)oxy)methyl)benzenesulfonamide

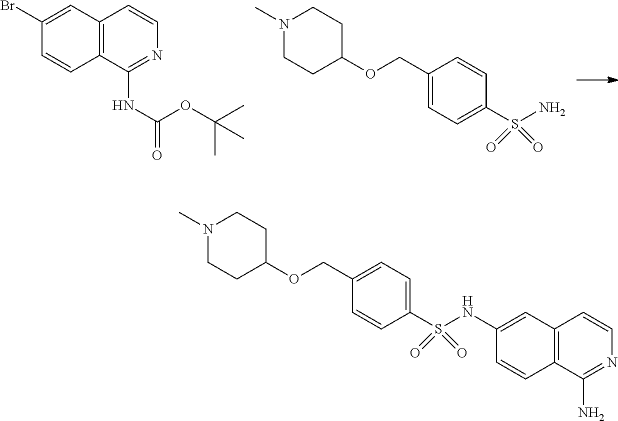 [SMILES: CC(C)(C)OC(=O)Nc1nccc2cc(Br)ccc12 ; CN1CCC(OCc2ccc(S(N)(=O)=O)cc2)CC1 ; CN1CCC(OCc2ccc(S(=O)(=O)Nc3ccc4c(N)nccc4c3)cc2)CC1]

Following General Method 4, tert-butyl (6-bromoisoquinolin-1-yl)carbamate (68 mg, 0.21 mmol) was reacted with 4-(((1-methylpiperidin-4-yl)oxy)methyl)benzenesulfonamide (60 mg, 0.21 mmol) and NaOtBu (41 mg, 0.43 mmol) in DMF at 40° C. for 18 h, using [tBuXPhos Pd(allyl)]OTf (15 mg, 0.02 mmol) as the ligand. The reaction was stirred at 80° C. for 12 h to cleave the boc protecting group. After quenching and elution through an SCX, the crude product was purified by flash chromatography (Silica, 0-20% (0.7M NH₃ in MeOH) in DCM) to afford the product (20 mg, 8% yield) as a cream solid.

[M+H]⁺=427.4

¹H NMR (500 MHz, DMSO-d6) 1.40-1.55 (2H, m), 1.76-1.88 (2H, m), 1.96-2.06 (2H, m), 2.14 (3H, s), 2.55-2.63 (2H, m), 3.25-3.42 (1H, m), 4.51 (2H, s), 6.69 (2H, s), 6.72 (1H, d, J=5.9 Hz), 7.16 (1H, dd, J=8.9, 2.2 Hz), 7.27 (1H, d, J=2.2 Hz), 7.43-7.50 (2H, m), 7.67 (1H, d, J=5.8 Hz), 7.74-7.82 (2H, m), 8.01 (1H, d, J=9.1 Hz), 10.60 (1H, br.s).

### Example Number 2197

### N5-(2-fluoro-4-(((1-methylpiperidin-4-yl)oxy)methyl)benzyl)isoquinoline-1,5-diamine

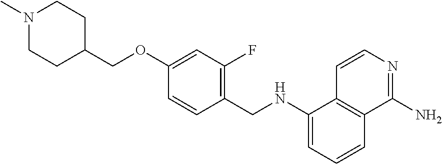 [SMILES: CN1CCC(COc2ccc(CNc3cccc4c(N)nccc34)c(F)c2)CC1]

### Methyl (5-((2-fluoro-4-(((1-methylpiperidin-4-yl)oxy)methyl)benzyl)amino)isoquinolin-1-yl)carbamate

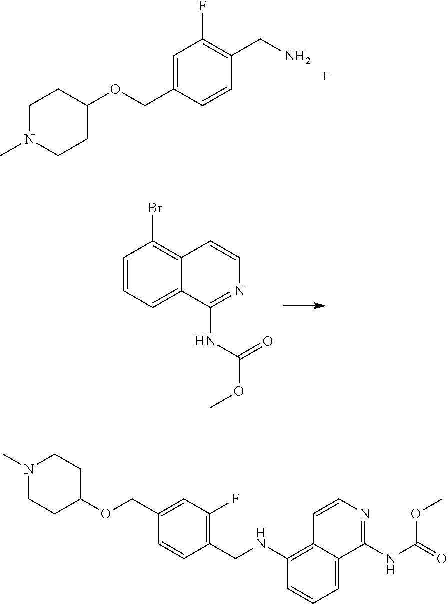

Following General Method 4, (2-fluoro-4-(((1-methylpiperidin-4-yl)oxy)methyl)phenyl)methanamine (150 mg, 0.59 mmol) was reacted with (5-bromoisoquinolin-1-yl)carbamate (121 mg, 0.43 mmol) and NaOtBu (83.0 mg, 0.86 mmol) in THF (6 mL) at 60° C. for 1 h. After quenching the reaction, the crude product was purified by flash chromatography (Silica, 0-20% (0.7 M NH₃ in MeOH) in DCM) to obtain the product (45 mg, 21% yield) as a cream solid.

[M+H]⁺=453.5

¹H NMR (500 MHz, DMSO-d6) δ 1.44-1.54 (2H, m), 1.79-1.85 (2H, m), 1.95-2.03 (2H, m), 2.12 (3H, s), 2.55-2.60 (3H, m), 3.66 (3H, s), 4.46 (2H, s), 4.53 (2H, d, J=5.3 Hz), 6.56 (1H, d, J=7.6 Hz), 7.06 (2H, d, J=8.1 Hz), 7.15 (1H, d, J=11.1 Hz), 7.25-7.28 (1H, m), 7.30-7.34 (2H, m), 7.99 (1H, d, J=6.1 Hz), 8.23 (1H, d, J=5.9 Hz), 9.86 (1H, s) ppm.

### N5-(2-fluoro-4-(((1-methylpiperidin-4-yl)oxy)methyl)benzyl)isoquinoline-1,5-diamine

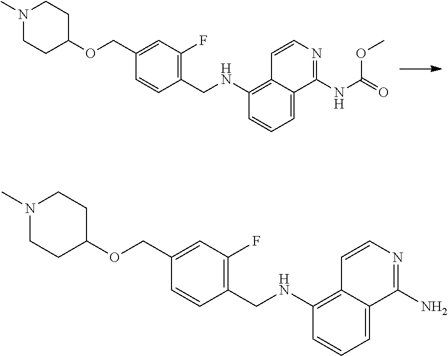

Deprotection of methyl (5-((2-fluoro-4-(((1-methylpiperidin-4-yl)oxy)methyl)benzyl)amino)isoquinolin-1-yl)carbamate (42 mg, 0.093 mmol) was completed using General Method 14a over 2 h. Following quenching and elution through an SCX, the product was lyophilised to afford the product (31 mg, 83% yield) as an off-white solid.

[M+H]⁺=395.4

¹H NMR (500 MHz, DMSO-d6) δ 1.43-1.55 (2H, m), 1.78-1.87 (2H, m), 1.94-2.02 (2H, m), 2.12 (3H, s), 2.55-2.60 (2H, m), 3.32-3.38 (1H, m), 4.46 (2H, s), 4.48 (2H, d, J=5.8 Hz), 6.44 (1H, d, J=7.7 Hz), 6.51 (2H, s), 6.69 (1H, t, J=5.9 Hz), 7.04-7.07 (1H, m), 7.12-7.16 (2H, m), 7.20 (1H, d, J=6.1 Hz), 7.27-7.31 (1H, m), 7.33 (1H, d, J=8.3 Hz), 7.75 (1H, d, J=6.1 Hz) ppm.

### Example Number 4260

### N5-((2-((1-methylpiperidin-4-yl)methoxy)pyridin-4-yl)methyl)isoquinoline-1,5-diamine

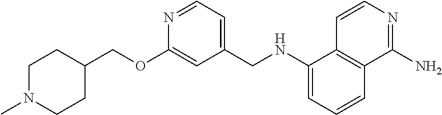 [SMILES: CN1CCC(COc2cc(CNc3cccc4c(N)nccc34)ccn2)CC1]

### Tert-butyl 4-(((4-cyanopyridin-2-yl)oxy)methyl)piperidine-1-carboxylate

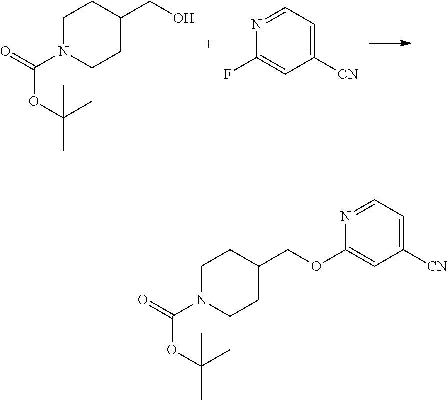 [SMILES: CC(C)(C)OC(=O)N1CCC(CO)CC1 ; N#Cc1ccnc(F)c1 ; CC(C)(C)OC(=O)N1CCC(COc2cc(C#N)ccn2)CC1]

Using General Method 1b, tert-butyl 4-(hydroxymethyl)piperidine-1-carboxylate (353 mg, 1.64 mmol) was reacted with 2-fluoroisonicotinonitrile (200 mg, 1.64 mmol) for 18 h. The reaction mixture was cooled to rt and diluted with water (10 mL). The crude product was extracted into DCM (2×25 mL), dried (MgSO₄), filtered and concentrated. The residue was purified by flash chromatography (Silica, 5-100% EtOAc in Pet. Ether) to afford the product (500 mg, 1.58 mmol, 96% yield) as a pale yellow oil.

[M-boc+H]⁺=218.1

¹H NMR (400 MHz, CDCl₃) δ 1.21-1.32 (2H, m), 1.47 (9H, s), 1.80 (2H, d, J=12.9 Hz), 1.92-2.02 (1H, m), 2.75 (2H, t, J=11.8 Hz), 4.09-4.20 (4H, m), 6.99 (1H, d, J=0.9 Hz), 7.07 (1H, dd, J=5.1, 1.3 Hz), 8.28 (1H, d, J=5.0 Hz) ppm.

### Tert-butyl 4-(((4-(aminomethyl)pyridin-2-yl)oxy)methyl)piperidine-1-carboxylate

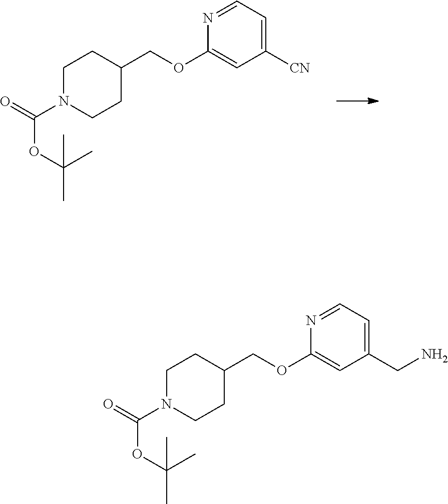 [SMILES: CC(C)(C)OC(=O)N1CCC(COc2cc(C#N)ccn2)CC1 ; CC(C)(C)OC(=O)N1CCC(COc2cc(CN)ccn2)CC1]

The nitrile, tert-butyl 4-(((4-cyanopyridin-2-yl)oxy)methyl)piperidine-1-carboxylate (500 mg, 1.58 mmol) was reduced according to General Method 3a using Raney Ni for 2 h. The solvent was removed in vacuo to afford the product (497 mg, 98% yield) as a colourless oil.

[M+H]⁺=322.1

¹H NMR (CDCl₃ 400 MHz) δ 1.25 (2H, qd, J=12.4, 4.4 Hz), 1.46 (9H, s), 1.73-1.83 (2H, m), 1.89-2.00 (1H, m), 2.33 (2H, br s), 2.73 (2H, t, J=12.8 Hz), 3.86 (2H, s), 4.04-4.19 (4H, m), 6.65-6.75 (1H, m), 6.77-6.88 (1H, m), 8.07 (1H, dd, J=5.3, 0.7 Hz) ppm

### Tert-butyl 4-(((4-(((1-((2,4-dimethoxybenzyl)amino)isoquinolin-5-yl)amino)methyl)pyridin-2-yl)oxy)methyl)piperidine-1-carboxylate

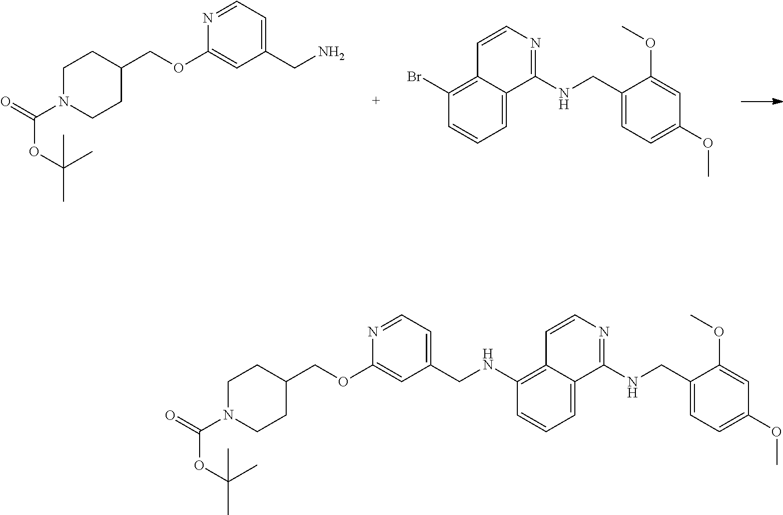 [SMILES: CC(C)(C)OC(=O)N1CCC(COc2cc(CN)ccn2)CC1 ; COc1ccc(CNc2nccc3c(Br)cccc23)c(OC)c1 ; COc1ccc(CNc2nccc3c(NCc4ccnc(OCC5CCN(C(=O)OC(C)(C)C)CC5)c4)cccc23)c(OC)c1]

Following General Method 4, tert-butyl 4-(((4-(aminomethyl)pyridin-2-yl)oxy)methyl)piperidine-1-carboxylate (497 mg, 1.55 mmol) was reacted with 5-bromo-N-(2,4-dimethoxybenzyl)isoquinolin-1-amine (635 mg, 1.7 mmol) and Cs₂CO₃ (1014 mg, 3.09 mmol) in 1,4-dioxane (6 mL) at 60° C. for 18 h. The reaction was cooled to rt and AcOH (177 μL, 3.09 mmol) was added. The reaction mixture was filtered through Celite®, washed with EtOAc (50 mL) and concentrated. The residue was purified by flash chromatography (Silica, 10-100% EtOAc in Pet. Ether) to afford the product (800 mg, 84% yield) as a pale yellow gum

[M+H]⁺=614.3

¹H NMR (400 MHz, CDCl₃) δ 0.83-0.97 (2H, m), 1.45 (9H, s), 1.59 (3H, s), 1.77-1.99 (3H, m), 2.72 (2H, t, J=12.3 Hz), 3.80 (3H, s), 3.86 (3H, s), 4.47 (2H, d, J=5.5 Hz), 4.72-4.78 (3H, m), 5.63 (1H, t, J=5.3 Hz), 6.44-6.55 (3H, m), 6.75 (1H, s), 6.85-6.90 (2H, m), 7.08 (1H, d, J=8.4 Hz), 7.20-7.32 (3H, m), 8.05 (1H, d, J=6.1 Hz), 8.09 (1H, d, J=5.4 Hz) ppm

### N1-(2,4-dimethoxybenzyl)-N5-((2-(piperidin-4-ylmethoxy)pyridin-4-yl)methyl)isoquinoline-1,5-diamine

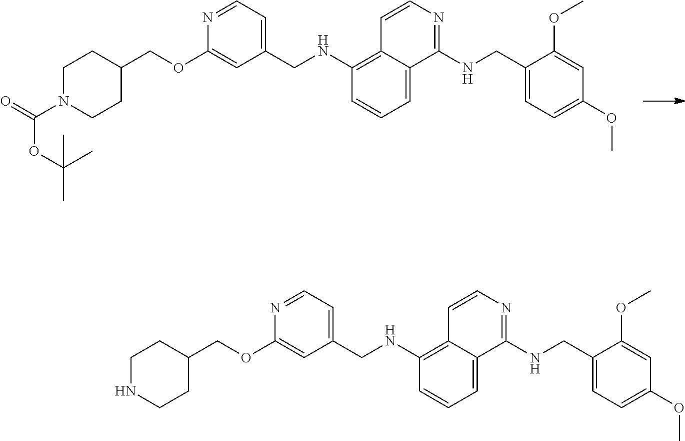 [SMILES: COc1ccc(CNc2nccc3c(NCc4ccnc(OCC5CCN(C(=O)OC(C)(C)C)CC5)c4)cccc23)c(OC)c1 ; COc1ccc(CNc2nccc3c(NCc4ccnc(OCC5CCNCC5)c4)cccc23)c(OC)c1]

Tert-butyl 4-(((4-(((1-((2,4-dimethoxybenzyl)amino)isoquinolin-5-yl)amino)methyl)pyridin-2-yl)oxy)methyl)piperidine-1-carboxylate (800 mg, 1.3 mmol) was deprotected following General Method 7a for 25 h. The reaction mixture was concentrated, converted to free base using a bicarbonate cartridge and triturated with Et₂O (20 mL) to afford the product (708 mg, 97% yield) as an orange oil.

[M+H]⁺=514.2

### N1-(2,4-dimethoxybenzyl)-N5-((2-((1-methylpiperidin-4-yl)methoxy)pyridin-4-yl)methyl)isoquinoline-1,5-diamine

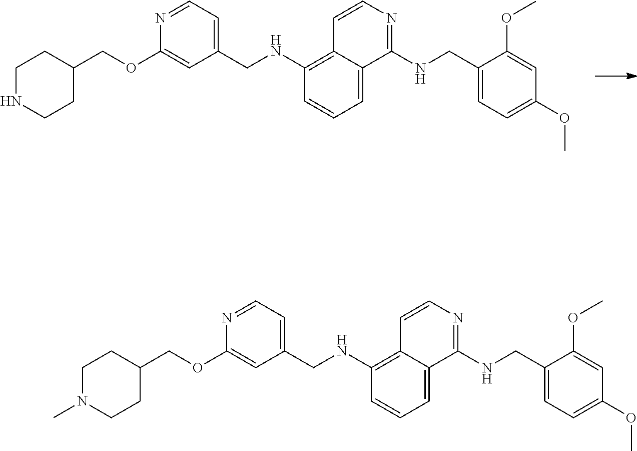

Following General Method 9, N1-(2,4-dimethoxybenzyl)-N5-((2-(piperidin-4-ylmethoxy)pyridin-4-yl)methyl)isoquinoline-1,5-diamine (1530 mg, 0.30 mmol) was reacted with formaldehyde (37% in water) (153 μL, 1.49 mmol). The crude product was purified by flash chromatography (Silica, 0-100% (2% NH₃ in EtOAc/MeCN/EtOH (3:3:1)) in Pet. Ether) to afford the product (95 mg, 54% yield) as a pale yellow gum.

¹H NMR (CDCl₃, 400 MHz) δ 1.35-1.45 (2H, m), 1.70-1.77 (1H, m), 1.79-1.87 (2H, m), 1.94 (2H, td, J=11.8, 2.5 Hz), 2.27 (3H, s), 2.86 (2H, d, J=11.6 Hz), 3.81 (3H, s), 3.86 (3H, s), 4.12 (2H, d, J=6.4 Hz), 4.46 (2H, d, J=5.6 Hz), 4.74 (3H, t, J=6.1 Hz), 5.63 (1H, t, J=5.3 Hz), 6.45 (1H, dd, J=8.2, 2.4 Hz), 6.50 (1H, d, J=2.4 Hz), 6.55 (1H, d, J=7.7 Hz), 6.75 (1H, dd, J=1.5, 0.8 Hz), 6.84-6.87 (1H, m), 6.88 (1H, dd, J=5.3, 1.5 Hz), 7.08 (1H, d, J=8.4 Hz), 7.22 (1H, t, J=8.0 Hz), 7.31 (1H, dd, J=8.2, 3.9 Hz), 8.04 (1H, d, J=6.1 Hz), 8.09 (1H, dd, J=5.3, 0.7 Hz) ppm.

### N5-((2-((1-methylpiperidin-4-yl)methoxy)pyridin-4-yl)methyl)isoquinoline-1,5-diamine

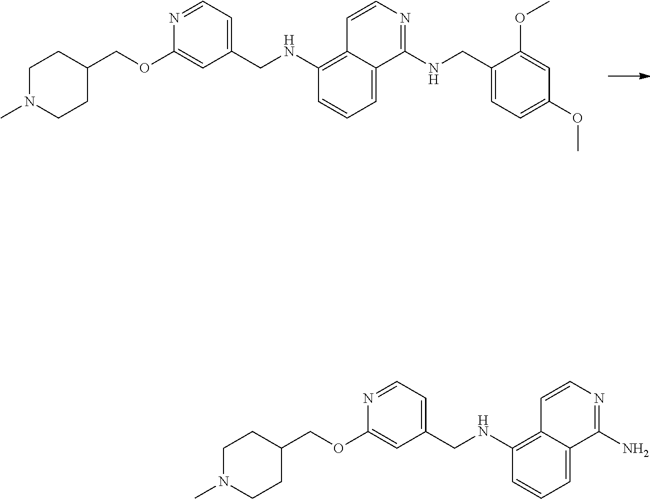

Deprotection of N1-(2,4-dimethoxybenzyl)-N5-((2-((1-methylpiperidin-4-yl)methoxy)pyridin-4-yl)methyl)isoquinoline-1,5-diamine (95 mg, 0.18 mmol) was carried out according to General Method 12, at rt for 1 h. The product was purified via automated prep HPLC (Mass directed 2-60% over 20 min in basic mobile phase) and lyophilised to afford the product (39 mg, 57% yield) as an off white solid.

[M+H]⁺=378.2

¹H NMR (DMSO, 400 MHz) δ 1.16-1.29 (2H, m), 1.57-1.70 (3H, m), 1.80 (2H, td, J=11.6, 2.3 Hz), 2.12 (3H, s), 2.72 (2H, dt, J=11.7, 3.2 Hz), 4.04 (2H, d, J=6.1 Hz), 4.43 (2H, d, J=6.0 Hz), 6.37 (1H, d, J=7.6 Hz), 6.51 (2H, s), 6.71 (1H, d, J=1.4 Hz), 6.79 (1H, t, J=6.1 Hz), 6.95 (1H, dd, J=5.3, 1.4 Hz), 7.11 (1H, t, J=8.0 Hz), 7.17-7.21 (1H, m), 7.33 (1H, d, J=8.3 Hz), 7.76 (1H, d, J=6.1 Hz), 8.03 (1H, dd, J=5.3, 0.6 Hz) ppm.

### Example Number 4261

### N6-((2-((1-methylpiperidin-4-yl)methoxy)pyridin-4-yl)methyl)isoquinoline-1,6-diamine

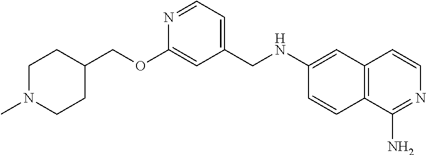 [SMILES: CN1CCC(COc2cc(CNc3ccc4c(N)nccc4c3)ccn2)CC1]

### Methyl (6-(((2-((1-methylpiperidin-4-yl)methoxy)pyridin-4-yl)methyl)amino)isoquinolin-1-yl)carbamate

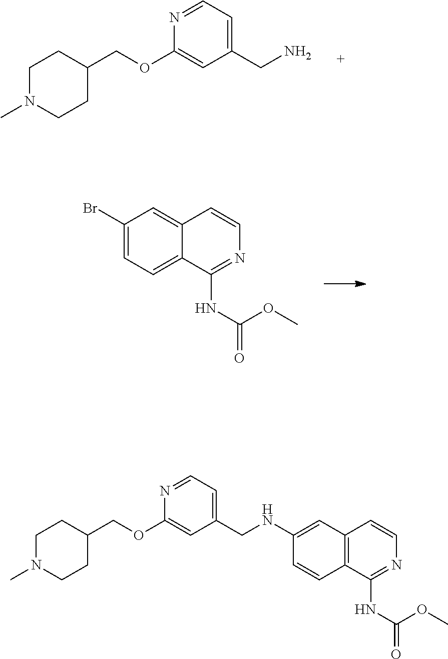

Using General Method 4, (6-bromoisoquinolin-1-yl)carbamate (119 mg, 0.435 mmol) was reacted with (2-((1-methylpiperidin-4-yl)methoxy)pyridin-4-yl)methanamine (100 mg, 0.43 mmol) and NaOtBu (82.0 mg, 0.85 mmol) in THF (6 mL) at 60° C. for 1 h. After quenching, the crude product was purified by flash chromatography (Silica, 0-20% (0.7M NH₃ in MeOH) in DCM) to afford the product (134 mg, 69% yield) as a cream solid.

[M+H]⁺=436.4

### N6-((2-((1-methylpiperidin-4-yl)methoxy)pyridin-4-yl)methyl)isoquinoline-1,6-diamine

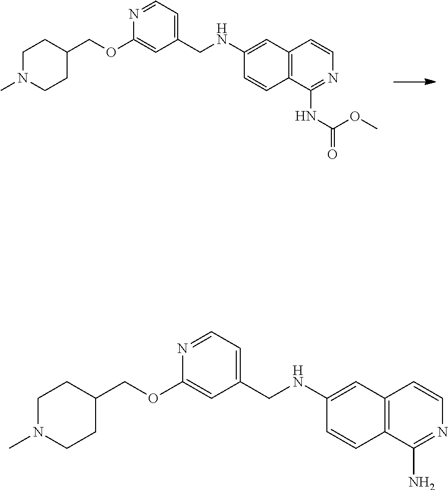

Methyl (6-(((2-((1-methylpiperidin-4-yl)methoxy)pyridin-4-yl)methyl)amino)isoquinolin-1-yl)carbamate (105 mg, 0.22 mmol) was deprotected using General Method 14a over 2 h. After quenching and elution through an SCX, the crude product was purified by flash chromatography (Silica, 0-20% (0.7M NH₃ in MeOH) in DCM) to afford the product (55 mg, 66% yield) as a colourless solid.

[M+H]⁺=378.5

¹H NMR (500 MHz, DMSO-d6) δ 1.17-1.30 (2H, m), 1.60-1.70 (3H, m), 1.77-1.86 (2H, m), 2.13 (3H, s), 2.70-2.78 (2H, m), 4.06 (2H, d, J=6.1 Hz), 4.37 (2H, d, J=6.2 Hz), 6.31 (2H, s), 6.42 (1H, d, J=2.3 Hz), 6.53 (1H, d, J=5.9 Hz), 6.75 (1H, s), 6.83 (1H, t, J=6.2 Hz), 6.85-6.90 (1H, m), 6.94-6.99 (1H, m), 7.54 (1H, d, J=5.8 Hz), 7.87 (1H, d, J=9.1 Hz), 8.06 (1H, d, J=5.3 Hz).

### Example Number 1017

### 1-(4-(((5-(((1-aminoisoquinolin-6-yl)amino)methyl)pyridin-2-yl)oxy)methyl)piperidin-1-yl)-2-methylpropan-2-ol

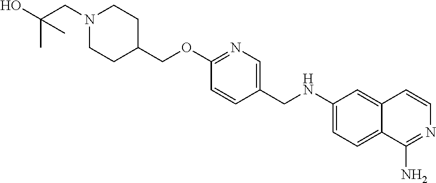 [SMILES: CC(C)(O)CN1CCC(COc2ccc(CNc3ccc4c(N)nccc4c3)cn2)CC1]

### Benzyl 4-(((5-bromopyridin-2-yl)oxy)methyl)piperidine-1-carboxylate

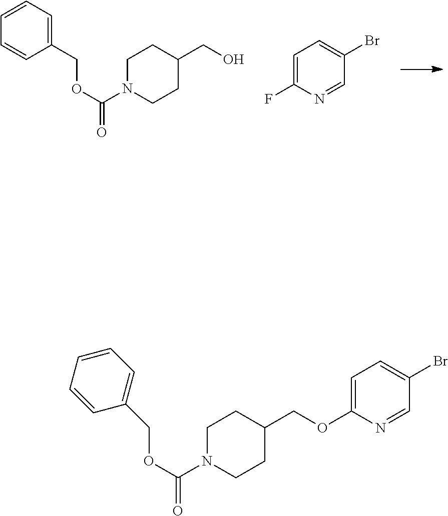 [SMILES: O=C(OCc1ccccc1)N1CCC(CO)CC1 ; Fc1ccc(Br)cn1 ; O=C(OCc1ccccc1)N1CCC(COc2ccc(Br)cn2)CC1]

Using General Method 1a, benzyl 4-(hydroxymethyl)piperidine-1-carboxylate (1.00 g, 4.01 mmol) was reacted with 5-bromo-2-fluoropyridine (413 μL, 4.01 mmol). The crude product was purified by flash chromatography (Silica, 0-30% EtOAc in isohexane) to afford the product (1.22 g, 71% yield) as a colourless gum which set on standing.

[M+H]⁺=405.0

¹H NMR (500 MHz, DMSO-d 6) 1.11-1.22 (2H, m), 1.74 (2H, d, J=13.0 Hz), 1.99 (2H, s), 2.72-2.93 (2H, m), 3.99-4.07 (1H, m), 4.10 (2H, d, J=6.5 Hz), 5.07 (2H, s), 6.80-6.84 (1H, m), 7.28-7.41 (5H, m), 7.89 (1H, dd, J=8.8, 2.6 Hz), 8.24-8.28 (1H, m)

### Benzyl 4-(((5-formylpyridin-2-yl)oxy)methyl)piperidine-1-carboxylate

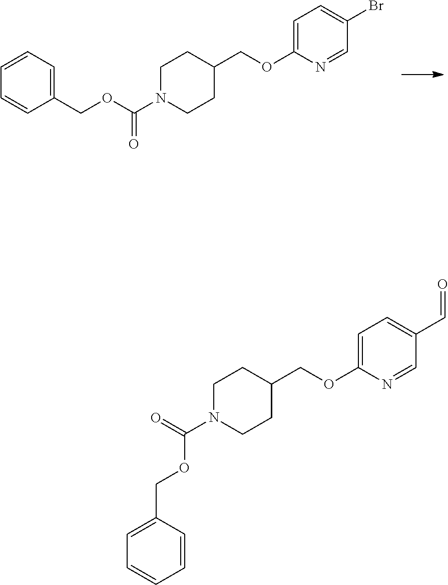 [SMILES: O=C(OCc1ccccc1)N1CCC(COc2ccc(Br)cn2)CC1 ; O=Cc1ccc(OCC2CCN(C(=O)OCc3ccccc3)CC2)nc1]

A solution of benzyl 4-(((5-bromopyridin-2-yl)oxy)methyl)piperidine-1-carboxylate (400 mg, 0.99 mmol), Et₃N (0.41 mL, 2.96 mmol), triethylsilane (0.47 mL, 2.96 mmol) and PdCl₂(dppf)-CH₂Cl₂ adduct (80 mg, 0.10 mmol) in DMF (6 mL) was sealed under an atmosphere of CO (1.5 bar) and heated at 90° C. for 4 h before being allowed to cool. The reaction mixture was taken up in EtOAc (40 mL) then washed with 1M HCl (aq) (40 mL), water/brine (1:1, 40 mL) and brine (40 mL). The organic phase was dried (MgSO₄), filtered and concentrated. The crude product was purified by chromatography (Silica, 0-40% EtOAc in isohexane) to afford the product (282 mg, 79% yield) as a colourless gum which set on standing.

[M+H]⁺=355.1

¹H NMR (500 MHz, DMSO-d6) 1.15-1.26 (2H, m), 1.71-1.81 (2H, m), 1.95-2.07 (1H, m), 2.73-2.95 (2H, m), 4.02-4.09 (2H, m), 4.26 (2H, d, J=6.5 Hz), 5.08 (2H, s), 6.99 (1H, d, J=8.6 Hz), 7.30-7.40 (5H, m), 8.12 (1H, dd, J=8.6, 2.4 Hz), 8.75 (1H, d, J=2.3 Hz), 9.96 (1H, s)

### Benzyl 4-(((5-(((1-(bis(tert-butoxycarbonyl)amino)isoquinolin-6-yl)amino)methyl)pyridin-2-yl)oxy)methyl)piperidine-1-carboxylate

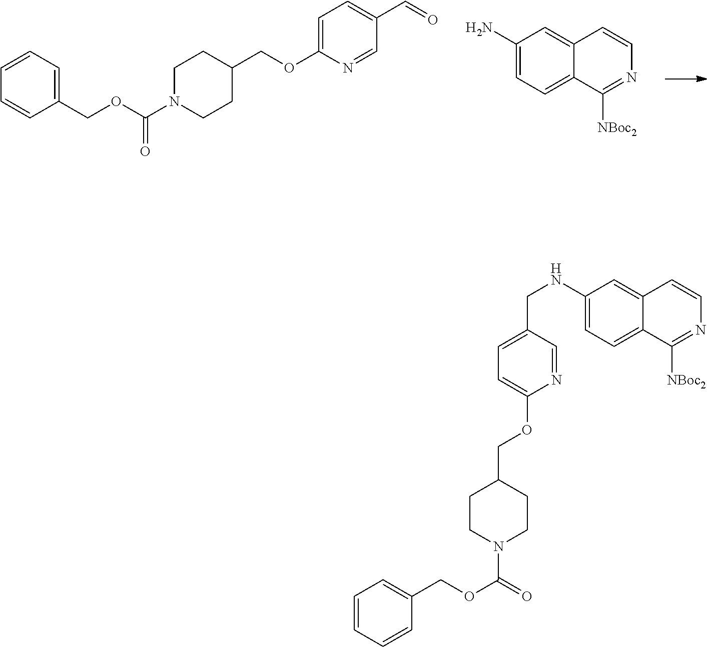

A solution of benzyl 4-(((5-formylpyridin-2-yl)oxy)methyl)piperidine-1-carboxylate (150 mg, 0.42 mmol), tert-butyl (6-aminoisoquinolin-1-yl)(tert-butoxycarbonyl)carbamate (150 mg, 0.42 mmol) and AcOH (23.9 μL, 0.42 mmol) in MeOH (5 mL) was treated with sodium cyanoborohydride (30 mg, 0.48 mmol) then heated to 70° C. for 3 h. The reaction was cooled to rt and concentrated. The residue was taken up in EtOAc (30 mL) and washed with NaHCO₃ (20 mL), water (20 mL) and brine (20 mL) before drying (MgSO₄), filtering and concentrating in vacuo. The crude product was purified by chromatography (Silica, 0-100% EtOAc in isohexane) to afford the product (132 mg, 45% yield) as a yellow foam.

[M+H]1=698.4

¹H NMR (500 MHz, DMSO-d 6) 1.10-1.22 (2H, m), 1.31 (18H, s), 1.67-1.79 (2H, m), 1.89-1.99 (1H, m), 2.70-2.93 (2H, m), 3.99-4.06 (2H, m), 4.09 (2H, d, J=6.5 Hz), 4.33 (2H, d, J=5.4 Hz), 5.07 (2H, s), 6.77 (1H, d, J=2.3 Hz), 6.80 (1H, d, J=8.5 Hz), 7.11-7.16 (2H, m), 7.29-7.39 (5H, m), 7.43 (1H, d, J=5.8 Hz), 7.50 (1H, d, J=9.1 Hz), 7.73 (1H, dd, J=8.5, 2.4 Hz), 8.07 (1H, d, J=5.8 Hz), 8.20 (1H, d, J=2.4 Hz).

### Tert-butyl (tert-butoxycarbonyl)(6-(((6-(piperidin-4-ylmethoxy)pyridin-3-yl)methyl)amino)isoquinolin-1-yl)carbamate

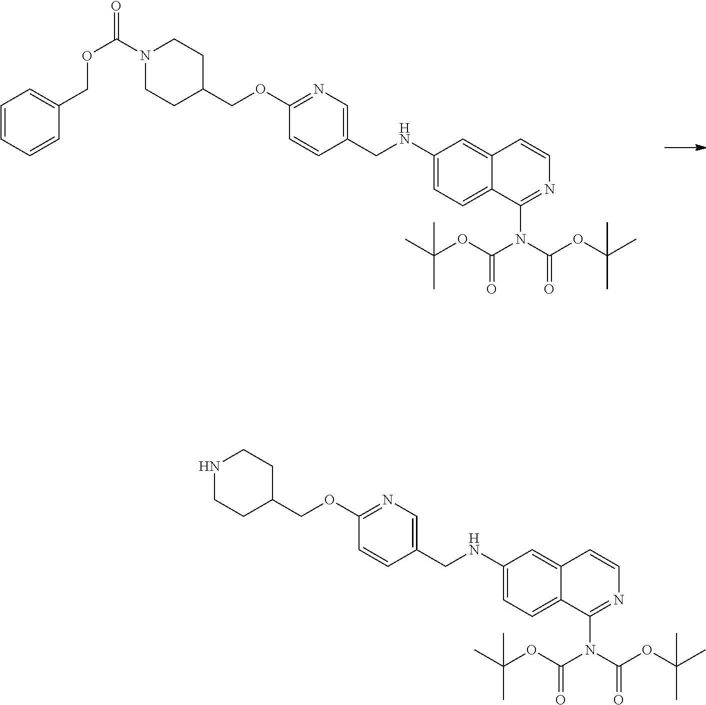 [SMILES: CC(C)(C)OC(=O)N(C(=O)OC(C)(C)C)c1nccc2cc(NCc3ccc(OCC4CCN(C(=O)OCc5ccccc5)CC4)nc3)ccc12 ; CC(C)(C)OC(=O)N(C(=O)OC(C)(C)C)c1nccc2cc(NCc3ccc(OCC4CCNCC4)nc3)ccc12]

A solution of benzyl 4-(((5-(((1-(bis(tert-butoxycarbonyl)amino)isoquinolin-6-yl)amino)methyl)pyridin-2-yl)oxy)methyl)piperidine-1-carboxylate (122 mg, 0.18 mmol) in MeOH (4 mL) was treated with 10% Pd/C (19 mg, 0.02 mmol) and sealed under an atmosphere of H₂ (2.5 bar). The reaction was heated at 50° C. for 2 h (4 bar). The reaction mixture was filtered through Celite® and concentrated. The crude product was purified by flash chromatography (Silica, 0-20% (0.7M NH₃ in MeOH) in DCM) to afford the product (65 mg, 65% yield) as a colourless solid.

[M+H]⁺=564.3

¹H NMR (500 MHz, DMSO-d6) 1.12-1.23 (2H, m), 1.31 (18H, s) 1.65-1.71 (2H, m), 1.79-1.88 (1H, m), 2.51-2.55 (2H, m), 2.95-3.01 (2H, m), 4.07 (2H, d, J=6.6 Hz), 4.34 (2H, d, J=5.5 Hz), 6.76-6.80 (2H, m), 7.12 (1H, t, J=5.8 Hz), 7.14-7.17 (1H, m), 7.43 (1H, d, J=5.8 Hz), 7.51 (1H, d, J=9.2 Hz), 7.72 (1H, dd, J=8.8, 2.5 Hz), 8.07 (1H, d, J=5.8 Hz), 8.20 (1H, d, J=2.3 Hz), NH not observed.

### Tert-butyl (tert-butoxycarbonyl)(6-(((6-((1-(2-hydroxy-2-methylpropyl)piperidin-4-yl)methoxy)pyridin-3-yl)methyl)amino)isoquinolin-1-yl)carbamate

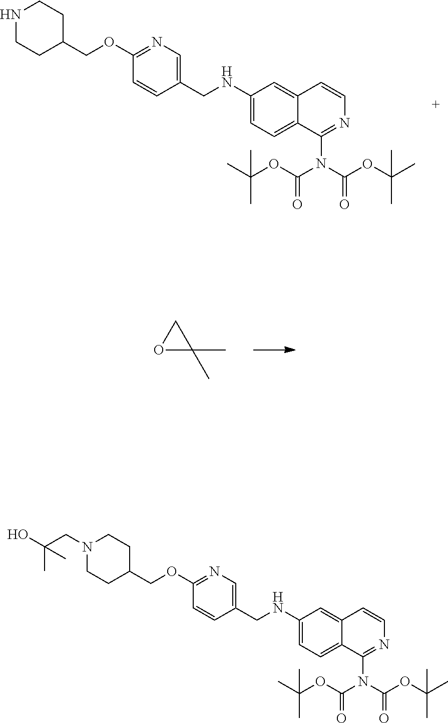

To a stirred suspension of tert-butyl (tert-butoxycarbonyl)(6-(((6-(piperidin-4-ylmethoxy)pyridin-3-yl)methyl)amino)isoquinolin-1-yl)carbamate (45 mg, 0.08 mmol) and K₂CO₃ (22 mg, 0.16 mmol) in DMF (1 mL) was added 2,2-dimethyloxirane (203 mg, 2.76 mmol) and the reaction heated at 40° C. for 4 days. The reaction mixture was diluted with EtOAc (30 mL) and washed with sat. Na₂CO₃ (aq) (20 mL), brine/water (1:1) (20 mL) and brine (20 mL) before drying (MgSO₄), filtering and concentrating in vacuo. The crude product was purified by flash chromatography (Silica, 0-20% (0.7M NH₃ in MeOH) in DCM) to afford the product (21 mg, 39% yield) as a colourless glass.

[M+H]⁺=636.6

### 1-(4-(((5-(((1-aminoisoquinolin-6-yl)amino)methyl)pyridin-2-yl)oxy)methyl)piperidin-1-yl)-2-methylpropan-2-ol

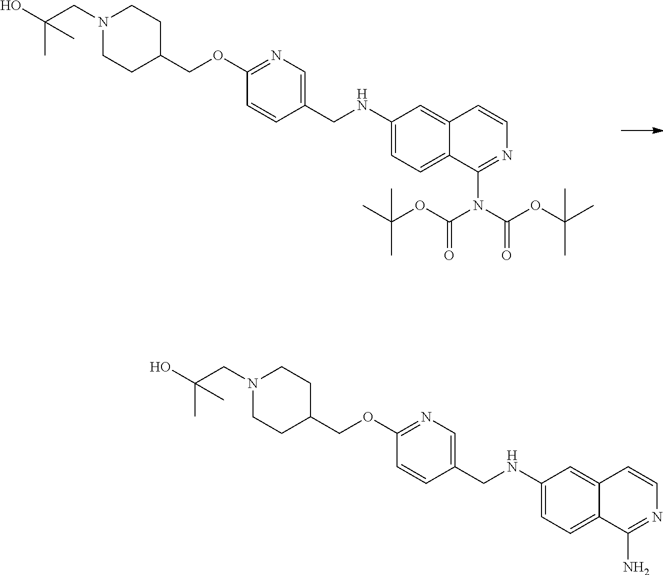 [SMILES: CC(C)(O)CN1CCC(COc2ccc(CNc3ccc4c(N(C(=O)OC(C)(C)C)C(=O)OC(C)(C)C)nccc4c3)cn2)CC1 ; CC(C)(O)CN1CCC(COc2ccc(CNc3ccc4c(N)nccc4c3)cn2)CC1]

Tert-butyl (tert-butoxycarbonyl)(6-(((6-((1-(2-hydroxy-2-methylpropyl)piperidin-4-yl)methoxy)pyridin-3-yl)methyl)amino)isoquinolin-1-yl)carbamate (21 mg, 0.033 mmol) was deprotected using General Method 7b. The crude product was purified by flash chromatography (Silica, 0-20% (0.7M NH₃ in MeOH) in DCM) and the product lyophilised to afford the product (13 mg, 89% yield) as a colourless solid.

[M+H]⁺=436.2

¹H NMR (500 MHz, DMSO-d6) δ 1.07 (6H, s), 1.22-1.34 (2H, m), 1.60-1.70 (3H, m), 2.05-2.13 (2H, m), 2.17 (2H, s), 2.89-2.96 (2H, m), 4.01 (1H, s), 4.07 (2H, d, J=6.1 Hz), 4.29 (2H, d, J=5.8 Hz), 6.36 (2H, s), 6.54 (1H, d, J=2.3 Hz), 6.58 (1H, d, J=6.1 Hz), 6.70 (1H, t, J=5.9 Hz), 6.75-6.80 (1H, m), 6.87 (1H, dd, J=9.0, 2.3 Hz), 7.55 (1H, d, J=5.9 Hz), 7.70 (1H, dd, J=8.5, 2.5 Hz), 7.86 (1H, d, J=9.0 Hz), 8.17 (1H, d, J=2.5 Hz).

### Example Number 1018

### 6-(2-(6-((1-methylpiperidin-4-yl)methoxy)pyridin-3-yl)ethyl)isoquinolin-1-amine

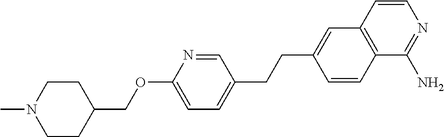 [SMILES: CN1CCC(COc2ccc(CCc3ccc4c(N)nccc4c3)cn2)CC1]

### 5-Ethynyl-2-((1-methylpiperidin-4-yl)methoxy)pyridine

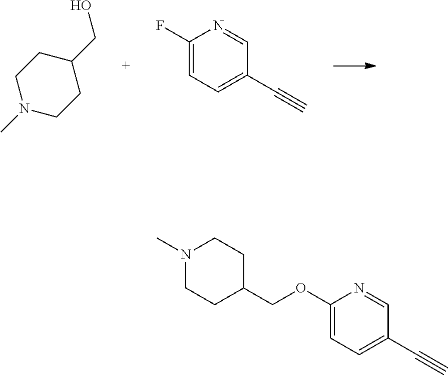

Following General Method 1b, 5-ethynyl-2-fluoropyridine (281 mg, 2.32 mmol) was reacted with (1-methylpiperidin-4-yl)methanol (300 mg, 2.32 mmol) at rt for 18 h. The reaction mixture was filtered over Celite® eluting with EtOAc and was concentrated. The crude product was purified by flash chromatography (Silica, 0-10% (0.7M NH₃ in MeOH) in DCM) to afford the product (266 mg, 49% yield) as a colourless solid.

[M+H]⁺=231.1

¹H NMR (500 MHz, DMSO-d6) 1.22-1.33 (2H, m), 1.65-1.73 (3H, m), 1.79-1.88 (2H, m), 2.14 (3H, s), 2.73-2.79 (2H, m), 4.12 (2H, d, J=6.2 Hz), 4.24 (1H, s), 6.83 (1H, dd, J=8.6, 0.8 Hz), 7.78 (1H, dd, J=8.6, 2.4 Hz), 8.29 (1H, d, J=2.2 Hz).

### 6-((6-((1-Methylpiperidin-4-yl)methoxy)pyridin-3-yl)ethynyl)isoquinolin-1-amine

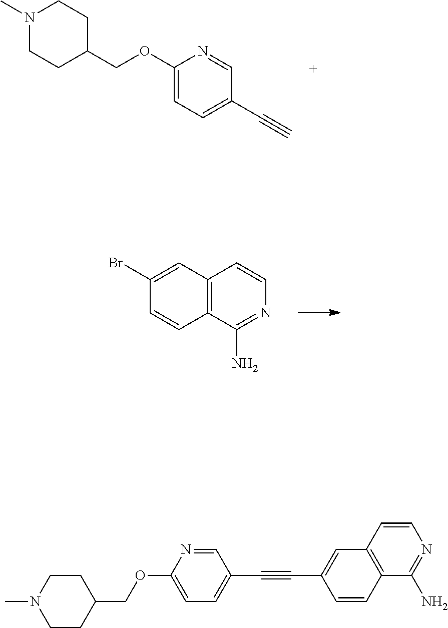

A solution of 5-ethynyl-2-((1-methylpiperidin-4-yl)methoxy)pyridine (125 mg, 0.54 mmol), 6-bromoisoquinolin-1-amine (145 mg, 0.65 mmol) and copper (1) iodide (6 mg, 0.003 mmol) in DMF (5 mL) was degassed with three vacuum N₂ (g) cycles before bubbling nitrogen through for 10 min. Pd(PPh₃)₄ (63 mg, 0.06 mmol) was added and the solution was degassed again with three vacuum N₂ (g) cycles and purged for a further 10 min with N₂ (g). The reaction was heated to 80° C. and stirred for 65 h. The reaction was cooled to rt and water (2 mL) and DCM (5 mL) was added. The crude reaction mixture was loaded onto an SCX in MeOH. The SCX was washed with MeOH (30 mL) and the product was eluted with 7M NH₃ in MeOH (50 mL). The resultant mixture was concentrated. The crude product was purified by flash chromatography (Silica, 0-20% (0.7M NH₃ in MeOH) in DCM) to afford the product (136 mg, 63% yield) as an orange solid.

[M+H]⁺=373.2

¹H NMR (500 MHz, DMSO-d6) 1.22-1.36 (2H, m), 1.66-1.78 (3H, m), 1.83-1.92 (2H, m), 2.17 (3H, s), 2.75-2.83 (2H, m), 4.16 (2H, d, J=6.1 Hz), 6.87 (2H, s), 6.92 (2H, d, J=5.8 Hz), 7.55 (1H, dd, J=8.6, 1.7 Hz), 7.84 (1H, d, J=5.8 Hz), 7.88-7.94 (2H, m), 8.22 (1H, d, J=8.6 Hz), 8.43 (1H, d, J=2.4 Hz).

### 6-(2-(6-((1-Methylpiperidin-4-yl)methoxy)pyridin-3-yl)ethyl)isoquinolin-1-amine

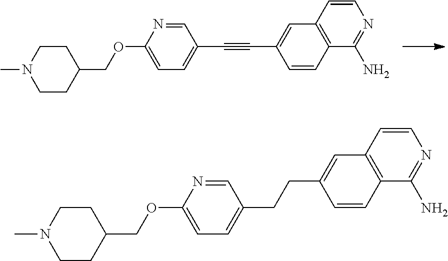 [SMILES: CN1CCC(COc2ccc(C#Cc3ccc4c(N)nccc4c3)cn2)CC1 ; CN1CCC(COc2ccc(CCc3ccc4c(N)nccc4c3)cn2)CC1]

To a solution of 6-((6-((1-methylpiperidin-4-yl)methoxy)pyridin-3-yl)ethynyl)isoquinolin-1-amine (135 mg, 0.36 mmol) in EtOH (5 mL) was added 10% Pd/C (60 mg, 0.06 mmol) and the reaction stirred at rt under H₂ (1 bar) in a steel-autoclave for 3 h. The crude reaction was filtered through Celite® and washed with EtOH (10 mL) before concentrating in vacuo. The crude product was purified by chromatography (Silica, 0-20% (0.7M NH₃ in MeOH) in DCM) to afford the product (58 mg, 41% yield) as a colourless solid.

[M+H]⁺=377.2

¹H NMR (500 MHz, DMSO-d6) 1.20-1.33 (2H, m), 1.62-1.74 (3H, m), 1.86-1.95 (2H, m), 2.18 (3H, s), 2.76-2.82 (2H, m), 2.88-2.94 (2H, m), 2.95-3.02 (2H, m), 4.04 (2H, d, J=6.1 Hz), 6.65-6.72 (3H, m), 6.80 (1H, d, J=5.8 Hz), 7.34 (1H, dd, J=8.5, 1.8 Hz), 7.46 (1H, d, J=1.8 Hz), 7.57 (1H, dd, J=8.5, 2.5 Hz), 7.74 (1H, d, J=5.8 Hz), 7.94 (1H, d, J=2.5 Hz), 8.09 (1H, d, J=8.5 Hz).

### Example Numbers 2198 and 2199

### N6-(2-fluoro-4-((((4S*,5R*)-2-methyl-2-azabicyclo[2.2.1]heptan-5-yl)oxy)methyl)benzyl)isoquinoline-1,6-diamine and N6-(2-fluoro-4-((((4R*,5R*)-2-methyl-2-azabicyclo[2.2.1]heptan-5-yl)oxy)methyl)benzyl)isoquinoline-1,6-diamine

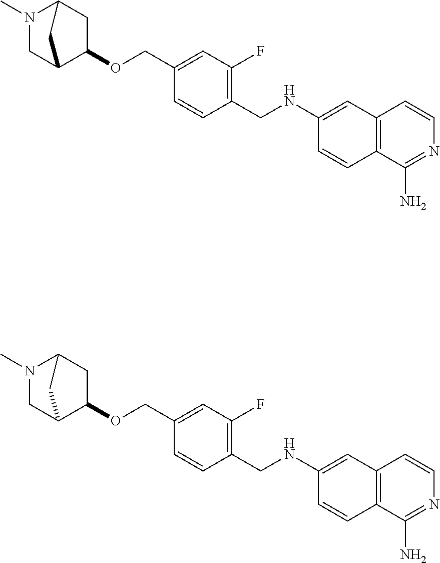

### Tert-butyl 5-((4-bromo-3-fluorobenzyl)oxy)-2-azabicyclo[2.2.1]heptane-2-carboxylate

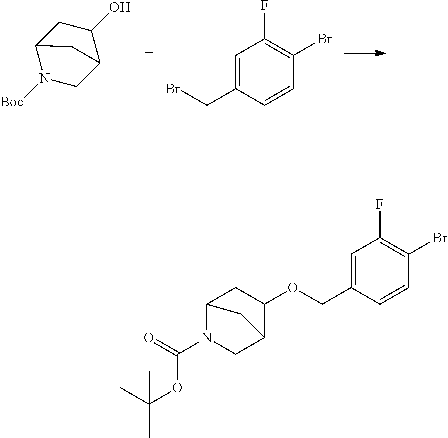 [SMILES: CC(C)(C)OC(=O)N1CC2CC1CC2O ; Fc1cc(CBr)ccc1Br ; CC(C)(C)OC(=O)N1CC2CC1CC2OCc1ccc(Br)c(F)c1]

Following General Method 5a, tert-butyl 5-hydroxy-2-azabicyclo[2.2.1]heptane-2-carboxylate (800 mg, 3.75 mmol) was reacted with 1-bromo-4-(bromomethyl)-2-fluorobenzene (1.00 g, 3.75 mmol) at rt for 16 h. The crude product was purified by flash chromatography (Silica, 0-50% EtOAc in isohexane) to afford the product (805 mg, 47% yield) as a thick colourless oil.

[M-^{t}Bu+H]⁺=344.0/346.0

¹H NMR (500 MHz, DMSO-d6) 1.34-1.41 (9H, m), 1.43-1.54 (2H, m), 1.60-1.66 (1H, m), 1.85-1.93 (1H, m), 2.62-2.67 (1H, m), 2.70-2.78 (1H, m), 3.06-3.16 (1H, m), 3.69-3.74 (1H, m), 4.02-4.08 (1H, m), 4.42-4.52 (2H, m), 7.13 (1H, dd, J=8.2, 1.9 Hz), 7.31 (1H, dd, J=9.9, 1.9 Hz), 7.64-7.70 (1H, m).F NMR (471 MHz, DMSO-d6) −108.60

### 5-((4-Bromo-3-fluorobenzyl)oxy)-2-methyl-2-azabicyclo[2.2.1]heptane

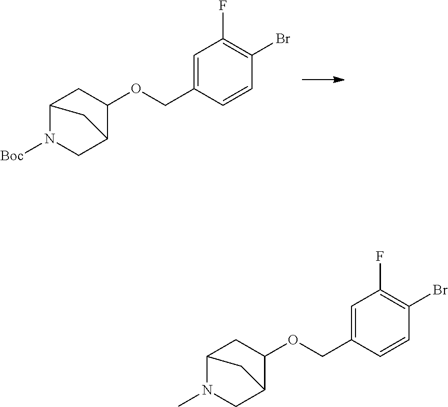 [SMILES: CC(C)(C)OC(=O)N1CC2CC1CC2OCc1ccc(Br)c(F)c1 ; CN1CC2CC1CC2OCc1ccc(Br)c(F)c1]

Using General Method 10, tert-butyl 5-((4-bromo-3-fluorobenzyl)oxy)-2-azabicyclo[2.2.1]heptane-2-carboxylate (800 mg, 2.00 mmol) was reacted for 3 h. After cooling to rt the reaction was treated with sat. Na₂CO₃ (aq) (50 mL) and extracted with EtOAc (3×30 mL). The organic phases were dried (MgSO₄), filtered and concentrated. The crude product was purified by flash chromatography (Silica, 0-50% EtOAc in isohexane) to afford the product (428 mg, 65% yield) as an off-white solid.[M+H]⁺=314.0/316.0

¹H NMR (500 MHz, DMSO-d 6) 1.21-1.29 (1H, m), 1.45-1.52 (2H, m), 1.79 (1H, d, J=9.5 Hz), 2.01-2.09 (1H, m), 2.13 (3H, s), 2.40-2.45 (1H, m), 2.62 (1H, dd, J=9.5, 4.4 Hz), 3.00-3.05 (1H, m), 3.48-3.54 (1H, m), 4.50-4.40 (2H, m), 7.12 (1H, dd, J=8.2, 1.9 Hz), 7.30 (1H, dd, J=9.9, 1.9 Hz), 7.64-7.70 (1H, m).

¹⁹F NMR (471 MHz, DMSO-d 6) −108.66.

### 2-Fluoro-4-(((2-methyl-2-azabicyclo[2.2.1]heptan-5-yl)oxy)methyl)benzonitrile

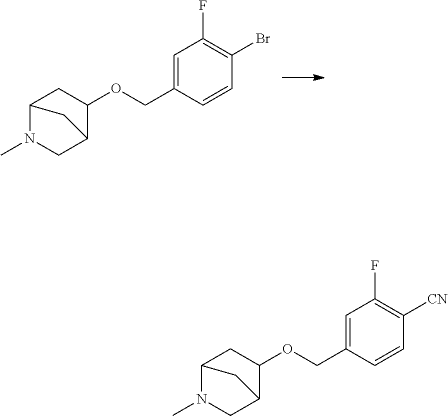 [SMILES: CN1CC2CC1CC2OCc1ccc(Br)c(F)c1 ; CN1CC2CC1CC2OCc1ccc(C#N)c(F)c1]

Using General Method 2, 5-((4-bromo-3-fluorobenzyl)oxy)-2-methyl-2-azabicyclo[2.2.1]heptane (480 mg, 1.53 mmol) was reacted for 88 h. concentrated. The crude product was purified by chromatography (Silica, 0-10% (0.7M NH₃ in MeOH) in DCM) to afford the product (177 mg, 29% yield) as a colourless oil.

[M+H]⁺=261.1

¹H NMR (500 MHz, DMSO-d 6) 1.23-1.31 (1H, m), 1.46-1.55 (2H, m), 1.79 (1H, d, J=9.5 Hz), 2.07 (1H, dd, J=13.6, 6.9 Hz), 2.13 (3H, s), 2.42-2.47 (1H, m), 2.62 (1H, dd, J=9.6, 4.5 Hz), 3.01-3.05 (1H, m), 3.54 (1H, d, J=6.9 Hz), 4.52-4.62 (2H, m), 7.34-7.37 (1H, m), 7.41-7.46 (1H, m), 7.88-7.93 (1H, m).

¹⁹F NMR (471 MHz, DMSO) δ −108.79.

### (2-Fluoro-4-(((2-methyl-2-azabicyclo[2.2.1]heptan-5-yl)oxy)methyl)phenyl)methanamine

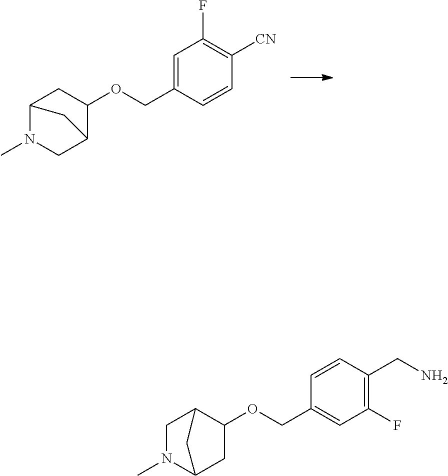 [SMILES: CN1CC2CC1CC2OCc1ccc(C#N)c(F)c1 ; CN1CC2CC1CC2OCc1ccc(CN)c(F)c1]

The nitrile, 2-fluoro-4-(((2-methyl-2-azabicyclo[2.2.1]heptan-5-yl)oxy)methyl)benzonitrile (50 mg, 0.19 mmol) was reduced using General Method 3a, over 1 h using a Raney Ni cartridge. The resultant solution was concentrated to give the product (45 mg, 75% yield) as a pale brown oil. [M+H]⁺=265.1

¹H NMR (500 MHz, DMSO-d 6) 1.21-1.28 (1H, m), 1.45-1.53 (2H, m), 1.80 (1H, d, J=9.6 Hz), 2.01-2.09 (1H, m), 2.14 (3H, s), 2.42-2.46 (1H, m), 2.63 (1H, dd, J=9.6, 4.4 Hz), 3.01-3.06 (1H, m), 3.50 (1H, dd, J=6.9, 2.4 Hz), 3.73 (2H, s), 4.35-4.50 (2H, m), 7.05 (1H, dd, J=11.1, 1.6 Hz), 7.10 (1H, dd, J=7.7, 1.6 Hz), 7.41-7.47 (1H, m). 2× exchangeable protons.

¹⁹F NMR (471 MHz, DMSO-d 6) −120.50.

### Methyl (6-((2-fluoro-4-((((4R*,5S*)-2-methyl-2-azabicyclo[2.2.1]heptan-5-yl)oxy)methyl)benzyl)amino)isoquinolin-1-yl)carbamate and

### methyl (6-((2-fluoro-4-((((4S*,5S*)-2-methyl-2-azabicyclo[2.2.1]heptan-5-yl)oxy)methyl)benzyl)amino)isoquinolin-1-yl)carbamate

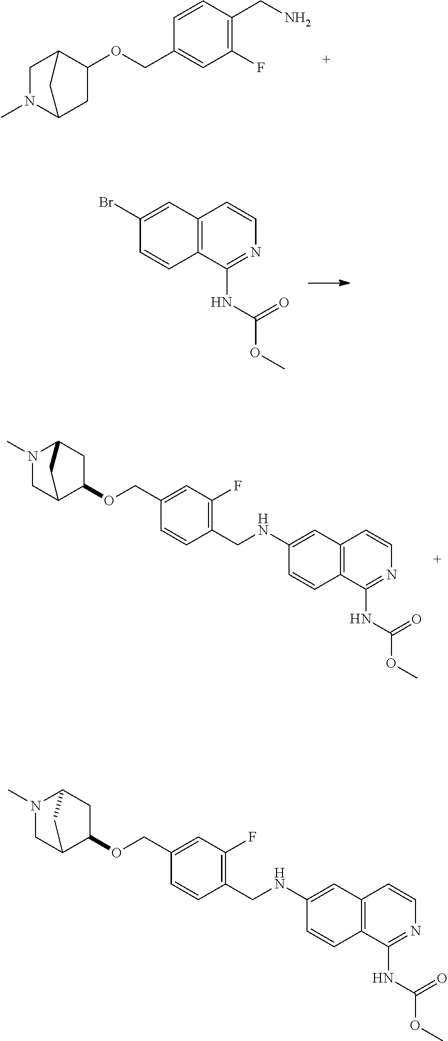

Following General Method 4, (2-fluoro-4-(((2-methyl-2-azabicyclo[2.2.1]heptan-5-yl)oxy)methyl)phenyl)methanamine (45 mg, 0.17 mmol) was reacted with methyl (6-bromoisoquinolin-1-yl)carbamate (48 mg, 0.17 mmol) and NaOtBu (2M in THF) (0.17 mL, 0.34 mmol) in THF (3 mL) at 60° C. for 2 h. After quenching and elution through an SCX, the crude product was purified by flash chromatography on silica gel (Silica, 0-20% (0.7M NH₃ in MeOH) in DCM) to isolate two separate diastereomers:

Methyl (6-((2-fluoro-4-((((4R*,5S*)-2-methyl-2-azabicyclo[2.2.1]heptan-5-yl)oxy)methyl)benzyl)amino)isoquinolin-1-yl)carbamate (13 mg, 16% yield) was isolated as a clear, colourless oil.

[M+H]⁺=465.2

Methyl (6-((2-fluoro-4-((((4S*,5S*)-2-methyl-2-azabicyclo[2.2.1]heptan-5-yl)oxy)methyl)benzyl)amino)isoquinolin-1-yl)carbamate (20 mg, 24% yield) was isolated as a clear, colourless oil.

[M+H]⁺=465.2

Stereochemistry is arbitrarily assigned for both diastereomers, relative and absolute configurations are unknown.

### N6-(2-fluoro-4-((((4R*,5S*)-2-methyl-2-azabicyclo[2.2.1]heptan-5-yl)oxy)methyl)benzyl)isoquinoline-1,6-diamine (Example Number 2199)

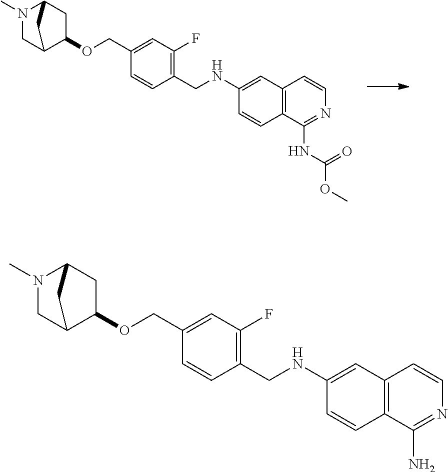

### Stereochemistry Currently not Defined

Following General Method 14a, methyl (6-((2-fluoro-4-((((4R*,5S*)-2-methyl-2-azabicyclo[2.2.1]heptan-5-yl)oxy)methyl)benzyl)amino)isoquinolin-1-yl)carbamate (13 mg, 0.03 mmol) was deprotected over 20 h. Following quenching, elution through an SCX and lyophilisation, the product was obtained (10 mg, 84% yield) as a colourless solid. The stereochemistry is arbitrarily assigned; the relative and absolute configurations are unknown.

[M+H]⁺=407.5

¹H NMR (500 MHz, DMSO-d 6) 1.23-1.31 (1H, m), 1.47-1.54 (2H, m), 1.81-1.89 (1H, m), 2.02-2.09 (1H, m), 2.17 (3H, s), 2.43-2.46 (1H, m), 2.60-2.69 (1H, m), 3.04-3.11 (1H, m), 3.49-3.54 (1H, m), 4.38 (2H, d, J=5.7 Hz), 4.39-4.48 (2H, m), 6.32 (2H, s), 6.48 (1H, d, J=2.4 Hz), 6.55 (1H, d, J=5.9 Hz), 6.72 (1H, t, J=6.0 Hz), 6.88 (1H, dd, J=9.1, 2.4 Hz), 7.09 (1H, dd, J=7.9, 1.6 Hz), 7.14 (1H, dd, J=11.1, 1.6 Hz), 7.34-7.39 (1H, m), 7.54 (1H, d, J=5.8 Hz), 7.86 (1H, d, J=9.1 Hz).

F NMR (471 MHz, DMSO-d 6) −119.12.

### N6-(2-fluoro-4-((((4R*,5R*)-2-methyl-2-azabicyclo[2.2.1]heptan-5-yl)oxy)methyl)benzyl)isoquinoline-1,6-diamine (Example Number 2199)

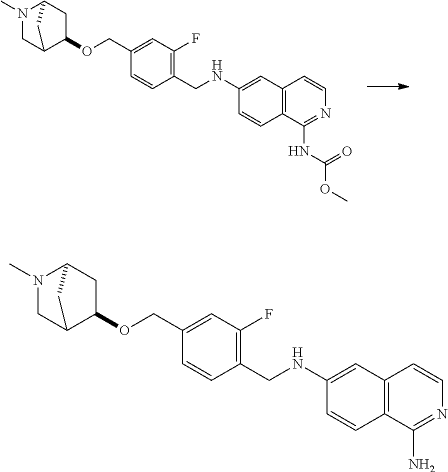

### Stereochemistry Currently not Defined

Deprotection of KOH methyl (6-((2-fluoro-4-((((4R*,5R*)-2-methyl-2-azabicyclo[2.2.1]heptan-5-yl)oxy)methyl)benzyl)amino)isoquinolin-1-yl)carbamate (20 mg, 0.43 mmol) was carried out using General Method 14a for 20 h. The product was isolated following elution through an SCX to obtain the product (18 mg, 98% yield) as a colourless solid. The stereochemistry is arbitrarily assigned; the relative and absolute configurations are unknown.

[M+H]⁺=407.5

¹H NMR (500 MHz, DMSO-d6) 1.21-1.27 (1H, m), 1.45-1.53 (2H, m), 1.78-1.84 (1H, m), 2.01-2.08 (1H, m), 2.15 (3H, s), 2.42-2.47 (1H, m), 2.62 (1H, s), 3.02-3.07 (1H, m), 3.47-3.53 (1H, m), 4.38 (2H, d, J=5.8 Hz), 4.39-4.48 (2H, m), 6.31 (2H, s), 6.48 (1H, d, J=2.3 Hz), 6.55 (1H, d, J=5.8 Hz), 6.71 (1H, t, J=6.0 Hz), 6.88 (1H, dd, J=9.0, 2.4 Hz), 7.09 (1H, dd, J=7.8, 1.6 Hz), 7.13 (1H, dd, J=11.1, 1.6 Hz), 7.34-7.39 (1H, m), 7.54 (1H, d, J=5.8 Hz), 7.86 (1H, d, J=9.0 Hz).

F NMR (471 MHz, DMSO-d6) −119.13.

### Example Number 4408

### N6-((2-((5,6,7,8-tetrahydroimidazo[1,2-a]pyridin-7-yl)methoxy)pyridin-4-yl)methyl)isoquinoline-1,6-diamine

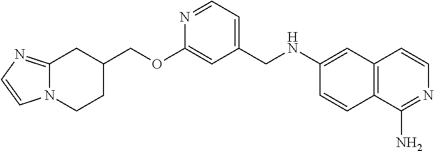 [SMILES: Nc1nccc2cc(NCc3ccnc(OCC4CCn5ccnc5C4)c3)ccc12]

### Methyl (6-(((2-((5,6,7,8-tetrahydroimidazo[1,2-a]pyridin-7-yl)methoxy)pyridin-4-yl)methyl)amino)isoquinolin-1-yl)carbamate

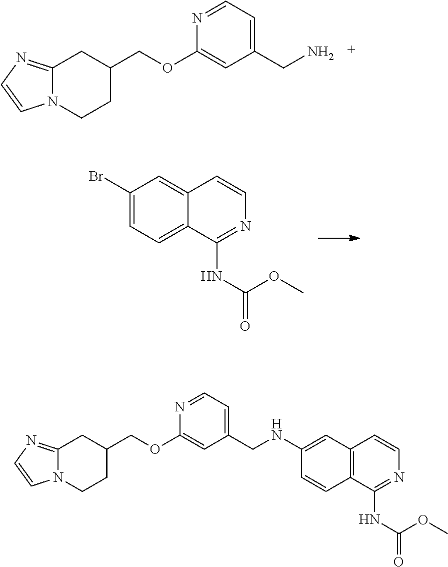 [SMILES: NCc1ccnc(OCC2CCn3ccnc3C2)c1 ; COC(=O)Nc1nccc2cc(Br)ccc12 ; COC(=O)Nc1nccc2cc(NCc3ccnc(OCC4CCn5ccnc5C4)c3)ccc12]

Using General Method 4, (2-((5,6,7,8-tetrahydroimidazo[1,2-a]pyridin-7-yl)methoxy)pyridin-4-yl)methanamine (75 mg, 0.29 mmol) was reacted with methyl (6-bromoisoquinolin-1-yl)carbamate (90 mg, 0.32 mmol) and NaOtBu (56 mg, 0.58 mmol) in THF (5 mL) at 60° C. for 3 h. After quenching the reaction mixture, the crude was purified by flash chromatography (Silica, 0-20% (0.7M NH₃ in MeOH) in DCM) to afford the product (90 mg, 66% yield) as an off-white solid. [M−H]⁻=457.2

### N6-((2-((5,6,7,8-tetrahydroimidazo[1,2-a]pyridin-7-yl)methoxy)pyridin-4-yl)methyl)isoquinoline-1,6-diamine

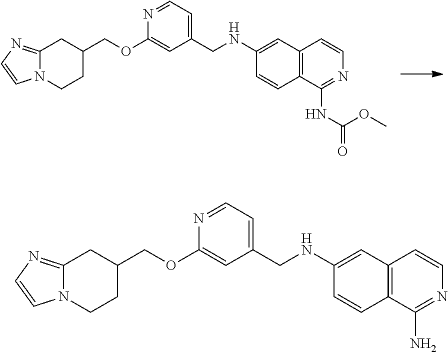 [SMILES: COC(=O)Nc1nccc2cc(NCc3ccnc(OCC4CCn5ccnc5C4)c3)ccc12 ; Nc1nccc2cc(NCc3ccnc(OCC4CCn5ccnc5C4)c3)ccc12]

Deprotection of methyl (6-(((2-((5,6,7,8-tetrahydroimidazo[1,2-a]pyridin-7-yl)methoxy)pyridin-4-yl)methyl)amino)isoquinolin-1-yl)carbamate (50 mg, 0.11 mmol) was performed using General Method 14a for 3 h. After quenching, elution through an SCX and lyophilisation, the product was isolated (34 mg, 76% yield) as an off white solid.

[M+H]⁺=401.2

¹H NMR (DMSO-d6, 400 MHz) δ 1.63-1.77 (1H, m), 2.06-2.15 (1H, m), 2.29-2.41 (1H, m), 2.43-2.49 (1H, m), 2.84-2.96 (1H, m), 3.79-3.92 (1H, m), 4.01-4.11 (1H, m), 4.24 (2H, d, J=6.6 Hz), 4.39 (2H, d, J=6.1 Hz), 6.31 (2H, s), 6.44 (1H, d, J=2.3 Hz), 6.53 (1H, d, J=5.9 Hz), 6.78-6.82 (2H, m), 6.85 (1H, t, J=6.2 Hz), 6.88 (1H, dd, J=9.0, 2.3 Hz), 6.95-7.09 (2H, m), 7.54 (1H, d, J=5.8 Hz), 7.87 (1H, d, J=9.0 Hz), 8.08 (1H, d, J=5.3 Hz)

### Example Number 1021

### N7-((6-((1-methylpiperidin-4-yl)methoxy)pyridin-3-yl)methyl)quinazoline-4,7-diamine

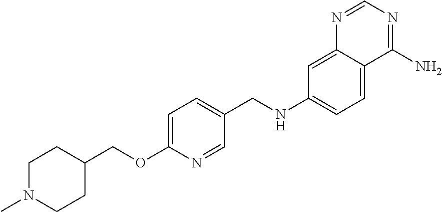 [SMILES: CN1CCC(COc2ccc(CNc3ccc4c(N)ncnc4c3)cn2)CC1]

### N7-((6-((1-methylpiperidin-4-yl)methoxy)pyridin-3-yl)methyl)quinazoline-4,7-diamine

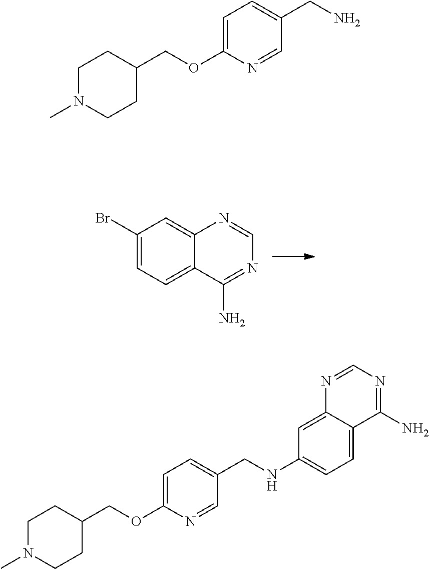 [SMILES: CN1CCC(COc2ccc(CN)cn2)CC1 ; Nc1ncnc2cc(Br)ccc12 ; CN1CCC(COc2ccc(CNc3ccc4c(N)ncnc4c3)cn2)CC1]

Following General Method 4, (6-((1-methylpiperidin-4-yl)methoxy)pyridin-3-yl)methanamine (75 mg, 0.32 mmol) was reacted with 7-bromoquinazolin-4-amine (70 mg, 0.31 mmol), and NaOtBu (60 mg, 0.62 mmol) in THF (4 mL) at 60° C. for 1 h. Following quenching, the crude product was purified by reverse phase flash chromatography (Silica C18, 5-50% (10 mM Ammonium Bicarbonate in MeCN) in water) to afford the product (19 mg, 15% yield) as a colourless solid after freeze drying.

¹H NMR (500 MHz, DMSO-d6) 1.21-1.31 (2H, m), 1.63-1.71 (3H, m), 1.79-1.86 (2H, m), 2.13 (3H, s), 2.72-2.77 (2H, m), 4.07 (2H, d, J=6.1 Hz), 4.30 (2H, d, J=5.8 Hz), 6.49 (1H, d, J=2.3 Hz), 6.78 (1H, d, J=8.5 Hz), 6.86 (1H, dd, J=8.9, 2.4 Hz), 6.92 (1H, t, J=5.8 Hz), 7.20 (2H, s), 7.69 (1H, dd, J=8.5, 2.4 Hz), 7.85 (1H, d, J=9.0 Hz), 8.13 (1H, s), 8.16 (1H, d, J=2.4 Hz)

[M+H]⁺=379.2

### Example Number 4265

### N6-((2-((1-methylpiperidin-4-yl)methoxy)-6-(trifluoromethyl)pyridin-4-yl)methyl)isoquinoline-1,6-diamine

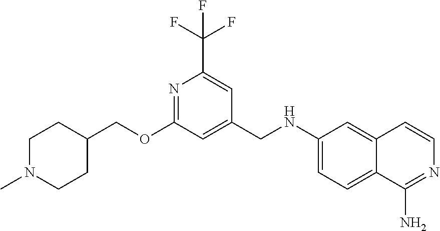 [SMILES: CN1CCC(COc2cc(CNc3ccc4c(N)nccc4c3)cc(C(F)(F)F)n2)CC1]

### Tert-butyl 4-(((4-cyano-6-(trifluoromethyl)pyridin-2-yl)oxy)methyl)piperidine-1-carboxylate

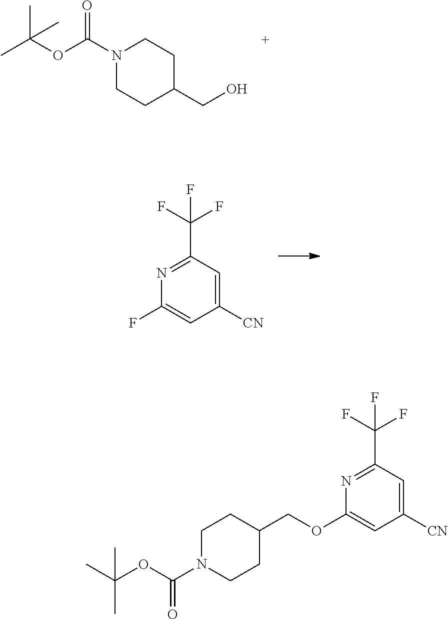 [SMILES: CC(C)(C)OC(=O)N1CCC(CO)CC1 ; N#Cc1cc(F)nc(C(F)(F)F)c1 ; CC(C)(C)OC(=O)N1CCC(COc2cc(C#N)cc(C(F)(F)F)n2)CC1]

Using General Method 1a, tert-butyl 4-(hydroxymethyl)piperidine-1-carboxylate (521 mg, 2.42 mmol) was reacted with 2-chloro-6-(trifluoromethyl)isonicotinonitrile (500 mg, 2.42 mmol) for 1.5 h. The crude product was purified by flash chromatography (Silica, 0-50% EtOAc in isohexane) to afford tert-butyl 4-(((4-cyano-6-(trifluoromethyl)pyridin-2-yl)oxy)methyl)piperidine-1-carboxylate (496 mg, 53% yield) as a colourless oil.

[M-boc+H]⁺=286.2

¹H NMR (500 MHz, DMSO-d6) δ 1.12-1.24 (2H, m), 1.40 (9H, s), 1.66-1.78 (2H, m), 1.90-2.05 (1H, m), 2.66-2.82 (2H, m), 3.91-4.05 (2H, m), 4.21 (2H, d, J=6.4 Hz), 7.80 (1H, s), 8.01 (1H, s).

### 2-((1-Methylpiperidin-4-yl)methoxy)-6-(trifluoromethyl)isonicotinonitrile

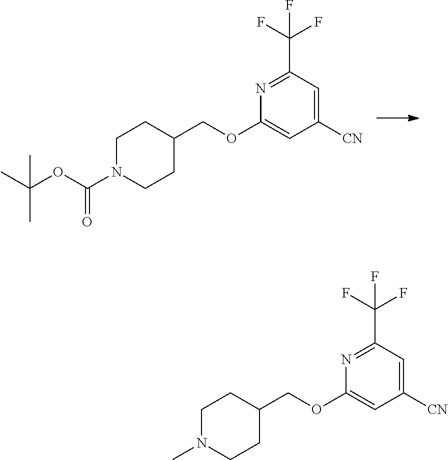 [SMILES: CC(C)(C)OC(=O)N1CCC(COc2cc(C#N)cc(C(F)(F)F)n2)CC1 ; CN1CCC(COc2cc(C#N)cc(C(F)(F)F)n2)CC1]

Tert-butyl 4-(((4-cyano-6-(trifluoromethyl)pyridin-2-yl)oxy)methyl)piperidine-1-carboxylate (480 mg, 1.26 mmol) was reacted according to General Method 10, at 90° C. for 18 h. After elution through an SCX and concentration, the product was isolated (255 mg, 72% yield) as a clear orange liquid.

[M+H]⁺=300.3

¹H NMR (500 MHz, DMSO-d6) δ 1.24-1.36 (2H, m), 1.67-1.78 (3H, m), 1.83-1.94 (2H, m), 2.16 (3H, s), 2.74-2.83 (2H, m), 4.19 (2H, d, J=6.2 Hz), 7.79 (1H, s), 8.01 (1H, s).

### (2-((1-Methylpiperidin-4-yl)methoxy)-6-(trifluoromethyl)pyridin-4-yl)methanamine

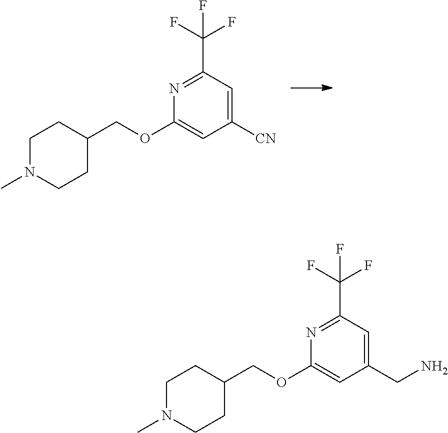 [SMILES: CN1CCC(COc2cc(C#N)cc(C(F)(F)F)n2)CC1 ; CN1CCC(COc2cc(CN)cc(C(F)(F)F)n2)CC1]

Reduction of 2-((1-methylpiperidin-4-yl)methoxy)-6-(trifluoromethyl)isonicotinonitrile (115 mg, 0.38 mmol) in MeOH (10 mL) was carried out following General Method 3a, using Raney Ni for 1.5 h. The resultant solution was concentrated under reduced pressure to afford the product (112 mg, 91% yield) as a colourless solid.

[M+H]⁺=304.3

¹H NMR (500 MHz, DMSO-d6) δ 1.27-1.40 (2H, m), 1.67-1.76 (3H, m), 1.86-1.97 (2H, m), 2.19 (3H, s), 2.78-2.86 (2H, m), 3.82 (2H, s), 4.13 (2H, d, J=6.0 Hz), 7.09 (1H, s), 7.48 (1H, s). NH₂ not observed.

### Tert-butyl (6-(((2-((1-methylpiperidin-4-yl)methoxy)-6-(trifluoromethyl)pyridin-4-yl)methyl)amino)isoquinolin-1-yl)carbamate

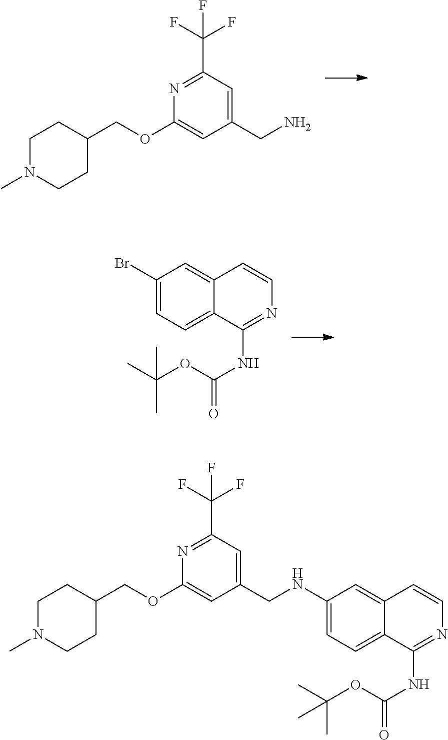 [SMILES: CN1CCC(COc2cc(CN)cc(C(F)(F)F)n2)CC1 ; CC(C)(C)OC(=O)Nc1nccc2cc(Br)ccc12 ; CN1CCC(COc2cc(CNc3ccc4c(NC(=O)OC(C)(C)C)nccc4c3)cc(C(F)(F)F)n2)CC1]

Following General Method 4, methyl tert-butyl (6-bromoisoquinolin-1-yl)carbamate (118 mg, 0.364 mmol) was reacted with (2-((1-methylpiperidin-4-yl)methoxy)-6-(trifluoromethyl)pyridin-4-yl)methanamine (100 mg, 0.330 mmol), NaOtBu (63 mg, 0.66 mmol) in THF (3 mL) at 60° C. for 1 h. The crude product was purified by flash chromatography (Silica, 0-20% (0.7M NH₃ in MeOH) in DCM) to afford the product (115 mg, 59% yield) as a colourless solid.

[M+H]⁺=546.4

### N6-((2-((1-methylpiperidin-4-yl)methoxy)-6-(trifluoromethyl)pyridin-4-yl)methyl)isoquinoline-1,6-diamine

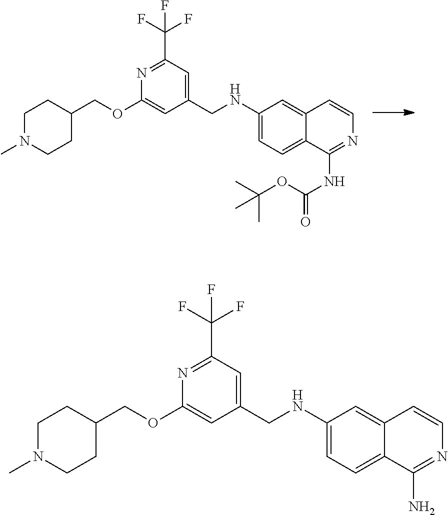 [SMILES: CN1CCC(COc2cc(CNc3ccc4c(NC(=O)OC(C)(C)C)nccc4c3)cc(C(F)(F)F)n2)CC1 ; CN1CCC(COc2cc(CNc3ccc4c(N)nccc4c3)cc(C(F)(F)F)n2)CC1]

Deprotection of tert-butyl (6-(((2-((1-methylpiperidin-4-yl)methoxy)-6-(trifluoromethyl)pyridin-4-yl)methyl)amino)isoquinolin-1-yl)carbamate (110 mg, 0.202 mmol) was carried out using General Method 7b, over 18 h at rt. The crude product was purified by flash chromatography (Silica, 0-20% (0.7M NH₃ in MeOH) in DCM) to afford the product (72 mg, 79% yield) as a colourless solid.

[M+H]⁺=446.4

¹H NMR (500 MHz, DMSO-d6) δ 1.20-1.32 (2H, m), 1.63-1.71 (3H, m), 1.78-1.85 (2H, m), 2.13 (3H, s), 2.71-2.77 (2H, m), 4.10 (2H, d, J=6.0 Hz), 4.49 (2H, d, J=6.2 Hz), 6.34 (2H, s), 6.46 (1H, s), 6.54 (1H, d, J=5.9 Hz), 6.86-6.92 (2H, m), 7.05 (1H, s), 7.48 (1H, s), 7.53-7.56 (1H, m), 7.89 (1H, d, J=9.0 Hz).

### Example Number 1026

### (6-(((6-((1-Methylpiperidin-4-yl)methoxy)pyridin-3-yl)methyl)amino)isoquinolin-4-yl)methanol

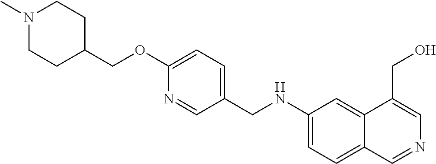 [SMILES: CN1CCC(COc2ccc(CNc3ccc4cncc(CO)c4c3)cn2)CC1]

### Methyl 6-(((6-((1-methylpiperidin-4-yl)methoxy)pyridin-3-yl)methyl)amino)isoquinoline-4-carboxylate

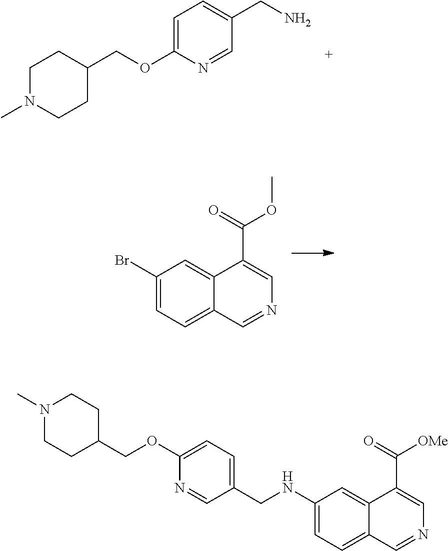 [SMILES: CN1CCC(COc2ccc(CN)cn2)CC1 ; COC(=O)c1cncc2ccc(Br)cc12 ; COC(=O)c1cncc2ccc(NCc3ccc(OCC4CCN(C)CC4)nc3)cc12]

Following General Method 4, (6-((1-methylpiperidin-4-yl)methoxy)pyridin-3-yl)methanamine (215 mg, 0.84 mmol) was reacted with methyl 6-bromoisoquinoline-4-carboxylate (224 mg, 0.84 mmol), and NaOtBu (2M in THF) (840 μL, 1.68 mmol) in THF (10 mL) at 60° C. for 1 h. After quenching the reaction, the crude product was purified by flash chromatography (Silica, 0-20% (0.7M NH₃ in MeOH) in DCM) to afford the product (200 mg, 40% yield) as a yellow solid.

[M+H]⁺=421.2

¹H NMR (500 MHz, DMSO-d6) 1.43-1.57 (2H, m), 1.89-1.97 (2H, m), 1.97-2.05 (1H, m), 2.71-2.81 (3H, m), 2.91-3.02 (2H, m), 3.42-3.48 (2H, m), 3.92 (3H, s), 4.13 (2H, d, J=6.3 Hz), 4.43 (2H, d, J=5.6 Hz), 6.83 (1H, d, J=8.5 Hz), 7.32 (1H, dd, J=9.0, 2.2 Hz), 7.71-7.75 (1H, m), 7.77 (1H, dd, J=8.5, 2.5 Hz), 8.01 (1H, d, J=9.0 Hz), 8.08-8.13 (1H, m), 8.24 (1H, d, J=2.5 Hz), 8.80 (1H, s), 9.13 (1H, s), 9.29 (1H, s)

### (6-(((6-((1-Methylpiperidin-4-yl)methoxy)pyridin-3-yl)methyl)amino)isoquinolin-4-yl)methanol

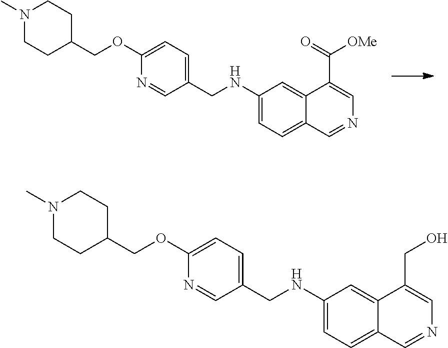 [SMILES: COC(=O)c1cncc2ccc(NCc3ccc(OCC4CCN(C)CC4)nc3)cc12 ; CN1CCC(COc2ccc(CNc3ccc4cncc(CO)c4c3)cn2)CC1]

Reduction of the ester, methyl 6-(((6-((1-methylpiperidin-4-yl)methoxy)pyridin-3-yl)methyl)amino)isoquinoline-4-carboxylate (45 mg, 0.70 mmol) was carried out using General Method 3b for 3 h. The crude product was purified by flash chromatography (Silica, 0-20% (0.7M NH₃ in MeOH) in DCM) to afford the product (9 mg, 22% yield) as an off-white solid.

[M+H]⁺=393.2

¹H NMR (500 MHz, DMSO-d 6) 1.20-1.32 (2H, m), 1.64-1.72 (3H, m), 1.79-1.87 (2H, m), 2.14 (3H, s), 2.72-2.79 (2H, m), 4.08 (2H, d, J=6.0 Hz), 4.34 (2H, d, J=5.6 Hz), 4.74 (2H, d, J=5.2 Hz), 5.18 (1H, t, J=5.4 Hz), 6.77-6.82 (2H, m), 7.05 (1H, t, J=5.7 Hz), 7.10 (1H, dd, J=8.9, 2.1 Hz), 7.73 (1H, dd, J=8.5, 2.5 Hz), 7.77 (1H, d, J=8.9 Hz), 8.17-8.23 (2H, m), 8.79 (1H, s).

### Example Number 1027

### N6-((2-methoxy-6-((1-methylpiperidin-4-yl)methoxy)pyridin-3-yl)methyl)isoquinoline-1,6-diamine

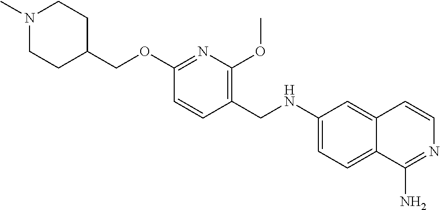 [SMILES: COc1nc(OCC2CCN(C)CC2)ccc1CNc1ccc2c(N)nccc2c1]

### 2-Methoxy-6-((1-methylpiperidin-4-yl)methoxy)nicotinonitrile

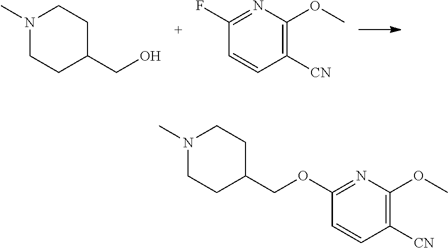

Following General Method 1a, (1-methylpiperidin-4-yl)methanol (382 mg, 2.96 mmol) was reacted with 6-fluoro-2-methoxynicotinonitrile (450 mg, 2.96 mmol). The crude product was purified by chromatography (Silica, 0-10% (0.7M NH₃ in MeOH) in DCM) to afford the product (200 mg, 25% yield) as an orange oil.

[M+H]⁺=262.3

¹H NMR (500 MHz, DMSO-d6) δ 1.23-1.33 (2H, m), 1.63-1.74 (3H, m), 1.81-1.93 (2H, m), 2.15 (3H, s), 2.72-2.83 (2H, m), 3.98 (3H, s), 4.21 (2H, d, J=6.2 Hz), 6.54 (1H, d, J=8.4 Hz), 8.07 (1H, d, J=8.4 Hz).

### (2-Methoxy-6-((1-methylpiperidin-4-yl)methoxy)pyridin-3-yl)methanamine

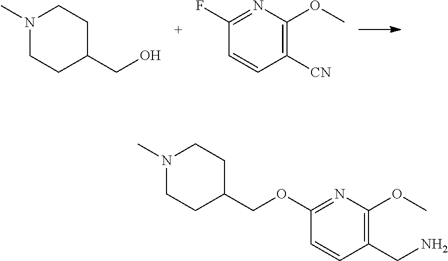

The nitrile, 2-methoxy-6-((1-methylpiperidin-4-yl)methoxy)nicotinonitrile (198 mg, 0.76 mmol) was reduced following General Method 3a, for 1.5 h using Raney Ni. The resultant solution was concentrated to afford the product (181 mg, 78% yield) as a colourless solid which was used without purification.

[M+H]⁺=266.6

¹H NMR (500 MHz, DMSO-d6) δ 1.22-1.32 (2H, m), 1.66-1.74 (3H, m), 1.79-1.87 (2H, m), 2.14 (3H, s), 2.73-2.80 (2H, m), 3.17 (2H, d, J=4.5 Hz), 3.32 (2H, s), 3.85 (3H, s), 4.08 (2H, d, J=6.1 Hz), 6.31 (1H, d, J=7.9 Hz), 7.60 (1H, d, J=7.9 Hz).

### Methyl (6-(((2-methoxy-6-((1-methylpiperidin-4-yl)methoxy)pyridin-3-yl)methyl)amino)isoquinolin-1-yl)carbamate

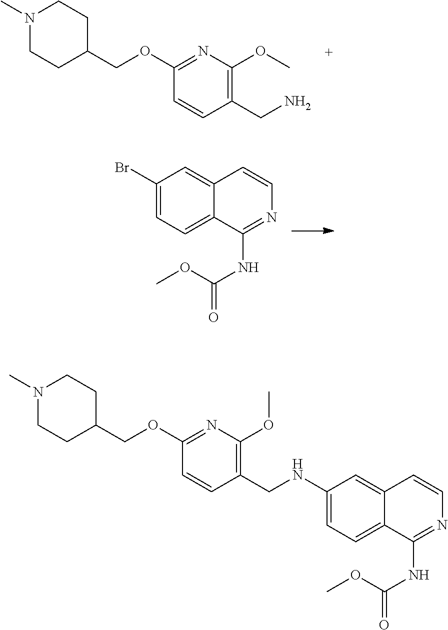 [SMILES: COc1nc(OCC2CCN(C)CC2)ccc1CN ; COC(=O)Nc1nccc2cc(Br)ccc12 ; COC(=O)Nc1nccc2cc(NCc3ccc(OCC4CCN(C)CC4)nc3OC)ccc12]

Using General Method 4, (6-bromoisoquinolin-1-yl)carbamate (158 mg, 0.56 mmol) was reacted with (2-methoxy-6-((1-methylpiperidin-4-yl)methoxy)pyridin-3-yl)methanamine (178 mg, 0.56 mmol) and NaOtBu (108 mg, 1.13 mmol) in THF (6 mL) at 60° C. for 1 h. After quenching, the crude product was purified by flash chromatography (Silica, 0-20% (0.7M NH₃ in MeOH) in DCM) to the product (184 mg, 67% yield) as a colourless solid.

[M+H]⁺=466.4

### N6-((2-methoxy-6-((1-methylpiperidin-4-yl)methoxy)pyridin-3-yl)methyl)isoquinoline-1,6-diamine

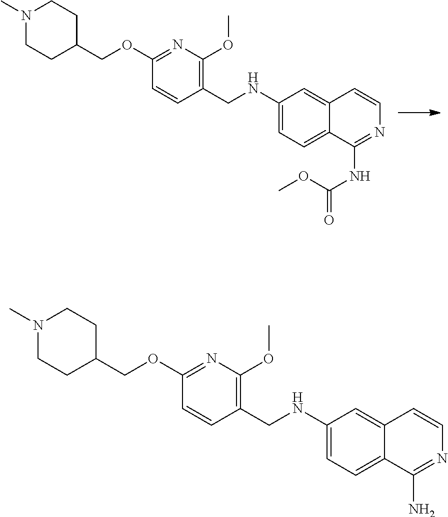 [SMILES: COC(=O)Nc1nccc2cc(NCc3ccc(OCC4CCN(C)CC4)nc3OC)ccc12 ; COc1nc(OCC2CCN(C)CC2)ccc1CNc1ccc2c(N)nccc2c1]

Deprotection of methyl (6-(((2-methoxy-6-((1-methylpiperidin-4-yl)methoxy)pyridin-3-yl)methyl)amino)isoquinolin-1-yl)carbamate (175 mg, 0.376 mmol) was carried out using general Method 14a, at 60° C. for 18 h. Following elution through an SCX, the crude product was purified by flash chromatography (Silica, 0-20% (0.7M NH₃ in MeOH) in DCM) to afford the product (130 mg, 83% yield) as a colourless solid.

[M+H]⁺=408.5

¹H NMR (500 MHz, DMSO-d6) δ 1.20-1.32 (2H, m), 1.63-1.74 (3H, m), 1.78-1.87 (2H, m), 2.14 (3H, s), 2.72-2.79 (2H, m), 3.93 (3H, s), 4.09 (2H, d, J=6.0 Hz), 4.19 (2H, d, J=5.6 Hz), 6.25-6.29 (2H, m), 6.32 (1H, d, J=8.0 Hz), 6.44 (1H, d, J=2.3 Hz), 6.53 (1H, t, J=5.9 Hz), 6.56 (1H, d, J=5.9 Hz), 6.85 (1H, dd, J=9.0, 2.4 Hz), 7.53-7.56 (2H, m), 7.84 (1H, d, J=9.0 Hz).

### Example Number 1028

### N6-((6-((2-(trifluoromethyl)-5,6,7,8-tetrahydroimidazo[1,2-a]pyridin-7-yl)methoxy)pyridin-3-yl)methyl)isoquinoline-1,6-diamine

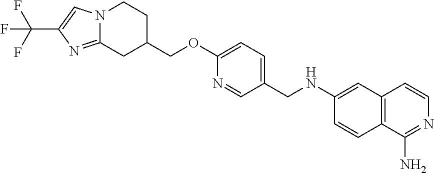 [SMILES: Nc1nccc2cc(NCc3ccc(OCC4CCn5cc(C(F)(F)F)nc5C4)nc3)ccc12]

### Ethyl 2-(trifluoromethyl)imidazo[1,2-a]pyridine-7-carboxylate

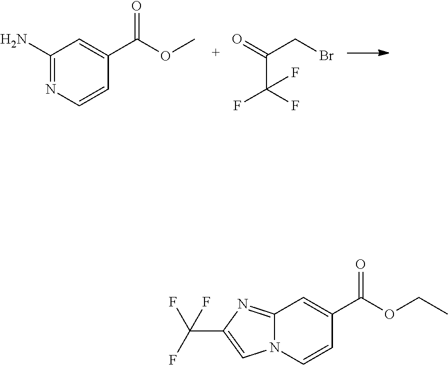

To a stirred suspension of methyl 2-aminoisonicotinate (3.05 g, 20.0 mmol) and K₂CO₃ (5.54 g, 40.1 mmol) in EtOH (120 mL) was added 3-bromo-1,1,1-trifluoropropan-2-one (2.7 mL, 26 mmol) and the resultant suspension was heated to 80° C. for 72 h. The reaction mixture was cooled, filtered and concentrated. The residue was dissolved in EtOH (120 mL), HCl (12M, 170 μL, 2.04 mmol) was added and the mixture heated at 70° C. overnight. The reaction was cooled to rt and filtered. The filtrate was concentrated and purified by flash chromatography (Silica, 0-5% (0.7 M NH₃ in MeOH) in DCM) to afford (1.37 g, 41% yield) as a pale yellow solid.

[M+H]⁺=259.3

### Ethyl 2-(trifluoromethyl)-5,6,7,8-tetrahydroimidazo[1,2-a]pyridine-7-carboxylate

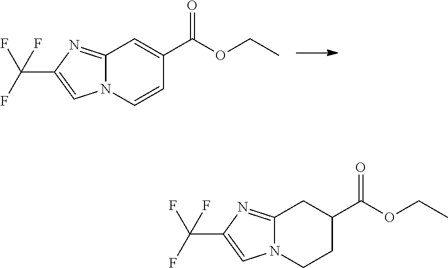 [SMILES: CCOC(=O)c1ccn2cc(C(F)(F)F)nc2c1 ; CCOC(=O)C1CCn2cc(C(F)(F)F)nc2C1]

Following General Method 3e, ethyl 2-(trifluoromethyl)imidazo[1,2-a]pyridine-7-carboxylate (1.37 g, 1.06 mmol) was reacted in EtOH (50 mL) and HCl (12M, 470 μL, 5.64 mmol) under 5 bar H₂ (g) at 70° C. for 3 h. The crude was partitioned between DCM (150 mL) and sat. aq. NaHCO₃ (150 mL), the aqueous was extracted with further DCM (150 mL) and the combined organics concentrated to afford the product (1.49 g, Quantitative yield) as a pale yellow solid.

### (2-(Trifluoromethyl)-5,6,7,8-tetrahydroimidazo[1,2-a]pyridin-7-yl)methanol

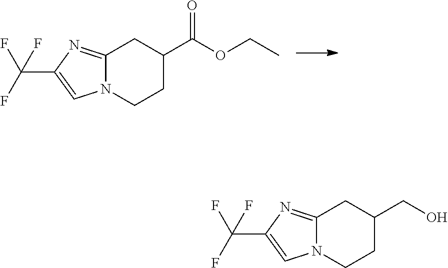 [SMILES: CCOC(=O)C1CCn2cc(C(F)(F)F)nc2C1 ; OCC1CCn2cc(C(F)(F)F)nc2C1]

Following General Method 3b, ethyl 2-(trifluoromethyl)-5,6,7,8-tetrahydroimidazo[1,2-a]pyridine-7-carboxylate (1.41 g, 3.23 mmol) was reacted for 30 min. The product was isolated (1.18 g, 93% yield) as a pale yellow solid and used without further purification.

[M+H]⁺=221.2

¹H NMR (500 MHz, DMSO-d6) 1.53-1.69 (1H, m), 1.93-2.10 (1H, m), 2.40 (1H, dd, J=16.7, 10.6 Hz), 2.85 (1H, ddd, J=16.7, 5.2, 1.6 Hz), 3.18 (1H, d, J=5.1 Hz), 3.36-3.48 (2H, m), 3.84-3.95 (1H, m), 4.05-4.18 (1H, m), 4.75 (1H, t, J=5.3 Hz), 7.64 (1H, s)

### 6-((2-(Trifluoromethyl)-5,6,7,8-tetrahydroimidazo[1,2-a]pyridin-7-yl)methoxy)nicotinonitrile

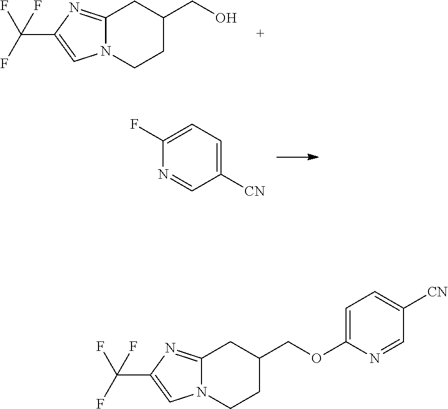 [SMILES: OCC1CCn2cc(C(F)(F)F)nc2C1 ; N#Cc1ccc(F)nc1 ; N#Cc1ccc(OCC2CCn3cc(C(F)(F)F)nc3C2)nc1]

Using General Method 1b, (2-(trifluoromethyl)-5,6,7,8-tetrahydroimidazo[1,2-a]pyridin-7-yl)methanol (400 mg, 1.82 mmol) was reacted with 6-fluoronicotinonitrile (266 mg, 2.18 mmol) for 22 h. The solids were removed by filtration and the filtrate concentrated. The crude product was purified by flash chromatography (Silica, 0-5% (0.7M NH₃ in MeOH) in DCM) to afford 6-((2-(trifluoromethyl)-5,6,7,8-tetrahydroimidazo[1,2-a]pyridin-7-yl)methoxy)nicotinonitrile (182 mg, 30% yield) as an off white solid.

[M+H]⁺=323.2

¹H NMR (500 MHz, DMSO-d6) 1.72-1.84 (1H, m), 2.12-2.20 (1H, m), 2.45-2.48 (1H, m), 2.59 (1H, dd, J=16.5, 10.9 Hz), 2.99 (1H, dd, J=16.5, 5.1 Hz), 3.91-4.01 (1H, m), 4.12-4.20 (1H, m), 4.39 (2H, d, J=6.5 Hz), 7.06 (1H, d, J=8.7 Hz), 7.68 (1H, s), 8.18 (1H, dd, J=8.6, 2.4 Hz), 8.71 (1H, d, J=2.3 Hz)

### (6-((2-(Trifluoromethyl)-5,6,7,8-tetrahydroimidazo[1,2-a]pyridin-7-yl)methoxy)pyridin-3-yl)methanamine

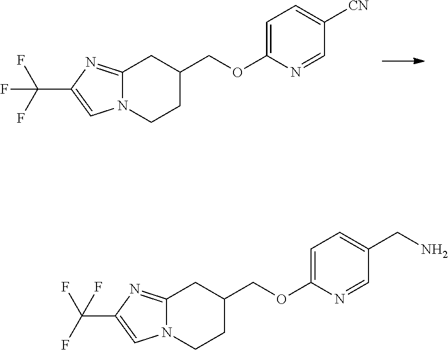 [SMILES: N#Cc1ccc(OCC2CCn3cc(C(F)(F)F)nc3C2)nc1 ; NCc1ccc(OCC2CCn3cc(C(F)(F)F)nc3C2)nc1]

Reduction of the nitrile, 6-((2-(trifluoromethyl)-5,6,7,8-tetrahydroimidazo[1,2-a]pyridin-7-yl)methoxy)nicotinonitrile (180 mg, 0.56 mmol) was carried out using General Method 3a, using a Raney Ni cartridge for 2 h. The reaction mixture was concentrated to afford the product (92 mg, 47% yield) as a pale yellow oil.

[M+H]⁺=327.3

¹H NMR (500 MHz, DMSO-d6) 1.70-1.85 (1H, m), 2.11-2.21 (1H, m), 2.36-2.46 (1H, m), 2.56 (1H, dd, J=16.6, 10.8 Hz), 2.97 (1H, ddd, J=16.6, 5.2, 1.5 Hz), 3.65 (2H, s), 3.91-4.00 (1H, m), 4.12-4.19 (1H, m), 4.26 (2H, d, J=6.6 Hz), 6.80 (1H, d, J=8.4 Hz), 7.67 (1H, d, J=1.4 Hz), 7.70 (1H, dd, J=8.5, 2.5 Hz), 8.06 (1H, d, J=2.4 Hz), (2× exchangable protons not seen).

### Methyl (6-(((6-((2-(trifluoromethyl)-5,6,7,8-tetrahydroimidazo[1,2-a]pyridin-7-yl)methoxy)pyridin-3-yl)methyl)amino)isoquinolin-1-yl)carbamate

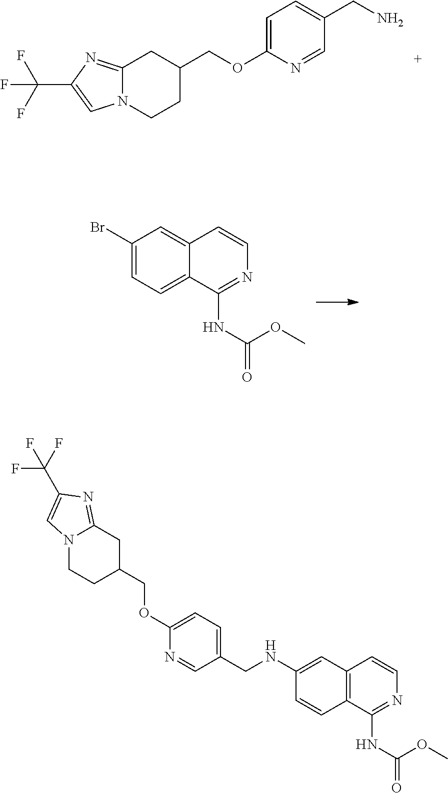 [SMILES: NCc1ccc(OCC2CCn3cc(C(F)(F)F)nc3C2)nc1 ; COC(=O)Nc1nccc2cc(Br)ccc12 ; COC(=O)Nc1nccc2cc(NCc3ccc(OCC4CCn5cc(C(F)(F)F)nc5C4)nc3)ccc12]

Using General Method 4, (6-((2-(trifluoromethyl)-5,6,7,8-tetrahydroimidazo[1,2-a]pyridin-7-yl)methoxy)pyridin-3-yl)methanamine (90 mg, 0.23 mmol), was reacted with methyl (6-bromoisoquinolin-1-yl)carbamate (66 mg, 0.23 mmol), and NaOtBu (45 mg, 0.47 mmol) in THF (2 mL) at 60° C. for 1 h. After quenching and concentrating, the crude product was purified by flash chromatography (Silica, 0-10% (0.7M NH₃ in MeOH) in DCM) to afford the product (54 mg, 42% yield) as a an off white solid.

[M+H]⁺=527.2

### N6-((6-((2-(trifluoromethyl)-5,6,7,8-tetrahydroimidazo[1,2-a]pyridin-7-yl)methoxy)pyridin-3-yl)methyl)isoquinoline-1,6-diamine

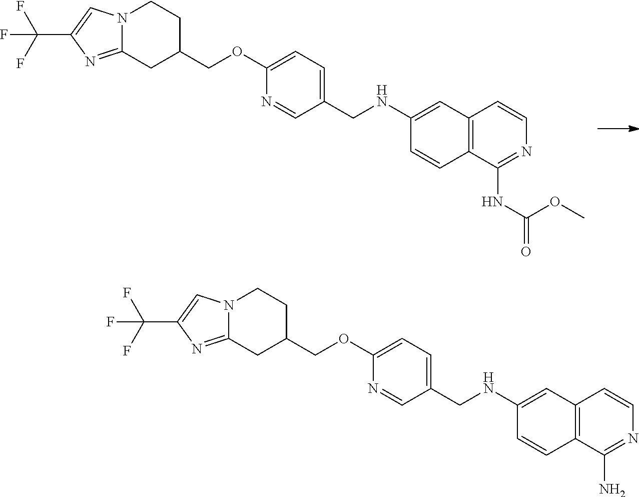 [SMILES: COC(=O)Nc1nccc2cc(NCc3ccc(OCC4CCn5cc(C(F)(F)F)nc5C4)nc3)ccc12 ; Nc1nccc2cc(NCc3ccc(OCC4CCn5cc(C(F)(F)F)nc5C4)nc3)ccc12]

Deprotection of methyl (6-(((6-((2-(trifluoromethyl)-5,6,7,8-tetrahydroimidazo[1,2-a]pyridin-7-yl)methoxy)pyridin-3-yl)methyl)amino)isoquinolin-1-yl)carbamate (50 mg, 0.10 mmol) was performed using General Method 14a for 72 h. After quenching and elution through an SCX, the crude product was purified by flash chromatography (Silica, 0-20% (0.7M NH₃ in MeOH) in DCM) to afford the product (15 mg, 32% yield) as an off white solid.

[M+H]⁺=469.4

¹H NMR (500 MHz, DMSO-d6) 1.69-1.81 (1H, m), 2.11-2.20 (1H, m), 2.39-2.47 (2H, m), 2.96 (1H, dd, J=16.4, 5.0 Hz), 3.91-4.00 (1H, m), 4.11-4.18 (1H, m), 4.26 (2H, d, J=6.5 Hz), 4.31 (2H, d, J=5.4 Hz), 6.50 (2H, s), 6.56 (1H, d, J=2.4 Hz), 6.60 (1H, d, J=6.0 Hz), 6.76-6.80 (1H, m), 6.84 (1H, d, J=8.5 Hz), 6.89 (1H, dd, J=9.0, 2.3 Hz), 7.54 (1H, d, J=6.0 Hz), 7.66 (1H, d, J=1.5 Hz), 7.74 (1H, dd, J=8.4, 2.5 Hz), 7.88 (1H, d, J=9.0 Hz), 8.20 (1H, d, J=2.4 Hz)

### Examples 1029 and 1030 (Enantiomers)

### N5-((6-((5,6,7,8-tetrahydroimidazo[1,2-a]pyridin-7-yl)methoxy)pyridin-3-yl)methyl)isoquinoline-1,5-diamine

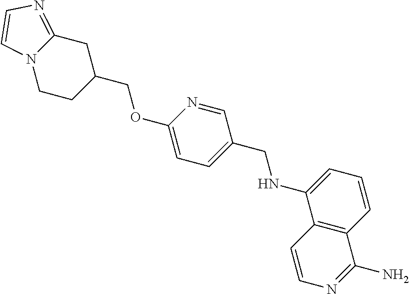 [SMILES: Nc1nccc2c(NCc3ccc(OCC4CCn5ccnc5C4)nc3)cccc12]

### Methyl (5-(((6-((5,6,7,8-tetrahydroimidazo[1,2-a]pyridin-7-yl)methoxy)pyridin-3-yl)methyl)amino)isoquinolin-1-yl)carbamate

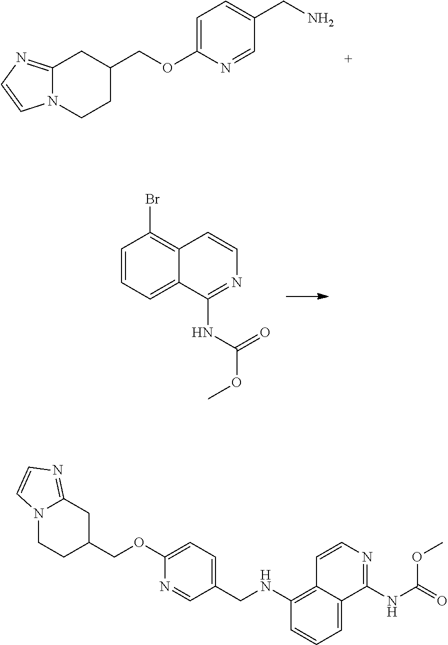 [SMILES: NCc1ccc(OCC2CCn3ccnc3C2)nc1 ; COC(=O)Nc1nccc2c(Br)cccc12 ; COC(=O)Nc1nccc2c(NCc3ccc(OCC4CCn5ccnc5C4)nc3)cccc12]

Following General Method 4, (6-((5,6,7,8-tetrahydroimidazo[1,2-a]pyridin-7-yl)methoxy)pyridin-3-yl)methanamine (190 mg, 0.74 mmol) was reacted with methyl (5-bromoisoquinolin-1-yl)carbamate (207 mg, 0.74 mmol) and NaOtBu (141 mg, 1.47 mmol) in THF (4 mL) at 60° C. for 1 h. After quenching, the crude product was purified by flash chromatography (Silica, 0-10% (0.7M NH₃ in MeOH) in DCM) to afford the product (179 mg, 53% yield) as an off-white solid.

[M+H]⁺=459.4

¹H NMR (500 MHz, DMSO-d6) δ 1.64-1.78 (1H, m), 2.07-2.16 (1H, m), 2.31-2.40 (1H, m), 2.44-2.51 (1H, m), 2.87-2.95 (1H, m), 3.66 (3H, s), 3.83-3.92 (1H, m), 4.06-4.12 (1H, m), 4.24 (2H, d, J=6.6 Hz), 4.44 (2H, d, J=5.8 Hz), 6.66 (1H, d, J=7.7 Hz), 6.79-6.83 (2H, m), 6.98 (1H, d, J=1.3 Hz), 7.02-7.07 (1H, m), 7.25 (1H, d, J=8.4 Hz), 7.30-7.35 (1H, m), 7.74 (1H, dd, J=8.5, 2.4 Hz), 7.96 (1H, d, J=6.0 Hz), 8.16-8.26 (2H, m), 9.85 (1H, s)

Methyl (5-(((6-((5,6,7,8-tetrahydroimidazo[1,2-a]pyridin-7-yl)methoxy)pyridin-3-yl)methyl)amino)isoquinolin-1-yl)carbamate (114 mg, 0.25 mmol) was submitted for chiral separation by chiral SFC on a Waters prep 15 with UV detection by DAD at 210 −400 nm, 40° C., 120 bar on a flow rate 15 mL/min using 50% of 1:1 MeOH: MeCN with 0.1% Ammonia to yield (R*)-(5-(((6-((5,6,7,8-tetrahydroimidazo[1,2-a]pyridin-7-yl)methoxy)pyridin-3-yl)methyl)amino)isoquinolin-1-yl)carbamate (30 mg, 0.062 mmol, 8.5% yield) as a white solid

[M+H]⁺=459.4

and methyl (S*)-(5-(((6-((5,6,7,8-tetrahydroimidazo[1,2-a]pyridin-7-yl)methoxy)pyridin-3-yl)methyl)amino)isoquinolin-1-yl)carbamate (28.5 mg, 0.057 mmol, 7.8% yield) as a white solid.

[M+H]⁺=459.4

### (R*)-N5-((6-((5,6,7,8-tetrahydroimidazo[1,2-a]pyridin-7-yl)methyl)isoquinoline-1,5-diamine (Example Number 1029)

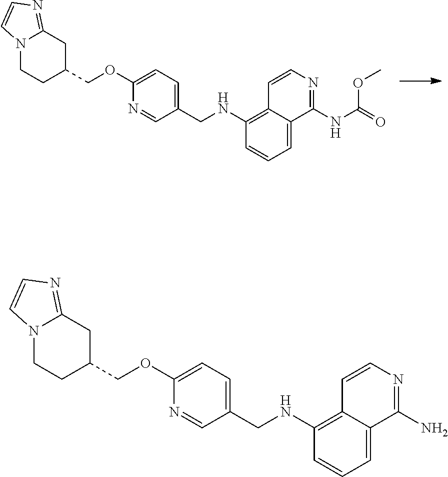

Deprotection of methyl (R*)-(5-(((6-((5,6,7,8-tetrahydroimidazo[1,2-a]pyridin-7-yl)methoxy)pyridin-3-yl)methyl)amino)isoquinolin-1-yl)carbamate (30 mg, 0.065 μmol) was carried out using General Method 14a over 20 h. After quenching and elution through an SCX, the crude product was purified by flash chromatography (Silica, 0-20% (0.7M NH₃ in MeOH) in DCM) to afford (R*)-N5-((6-((5,6,7,8-tetrahydroimidazo[1,2-a]pyridin-7-yl)methoxy)pyridin-3-yl)methyl)isoquinoline-1,5-diamine (20 mg, 73% yield) as an off white solid.

[M+H]⁺=401.2

¹H NMR (500 MHz, DMSO-d6) δ 1.66-1.78 (1H, m), 2.10-2.16 (1H, m), 2.32-2.42 (1H, m), 2.47-2.54 (1H, m), 2.89-2.98 (1H, m), 3.85-3.94 (1H, m), 4.05-4.12 (1H, m), 4.24 (2H, d, J=6.6 Hz), 4.40 (2H, d, J=5.7 Hz), 6.58 (1H, d, J=7.8 Hz), 6.66 (2H, s), 6.71 (1H, t, J=6.0 Hz), 6.80 (1H, d, J=8.5 Hz), 6.86 (1H, d, J=1.3 Hz), 7.03 (1H, d, J=1.3 Hz), 7.17 (1H, app t, J=8.0 Hz), 7.20 (1H, d, J=6.2 Hz), 7.35 (1H, d, J=8.3 Hz), 7.70-7.75 (2H, m), 8.19 (1H, d, J=2.4 Hz)

### (S*)-N5-((6-((5,6,7,8-tetrahydroimidazo[1,2-a]pyridin-7-yl)methyl)isoquinoline-1,5-diamine (Example Number 1030)

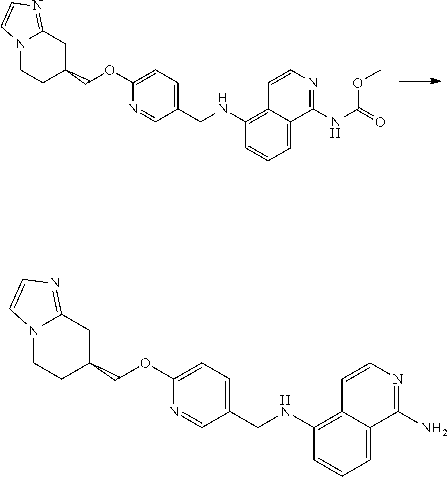 [SMILES: COC(=O)Nc1nccc2c(NCc3ccc(OC[C@H]4CCn5ccnc5C4)nc3)cccc12 ; Nc1nccc2c(NCc3ccc(OC[C@H]4CCn5ccnc5C4)nc3)cccc12]

Deprotection of methyl (S*)-(5-(((6-((5,6,7,8-tetrahydroimidazo[1,2-a]pyridin-7-yl)methoxy)pyridin-3-yl)methyl)amino)isoquinolin-1-yl)carbamate (25 mg, 0.055 mmol) was carried out using General method 14a over 20 h. After quenching and elution through an SCX, the crude product was purified by flash chromatography (Silica, 0-20% (0.7M NH₃ in MeOH) in DCM) to afford (S*)-N5-((6-((5,6,7,8-tetrahydroimidazo[1,2-a]pyridin-7-yl)methoxy)pyridin-3-yl)methyl)isoquinoline-1,5-diamine (19 mg, 84% yield) as an off white solid.

[M+H]⁺=401.4

¹H NMR (500 MHz, DMSO-d6) δ 1.65-1.78 (1H, m), 2.10-2.16 (1H, m), 2.31-2.42 (1H, m), 2.46-2.54 (1H, m), 2.88-2.97 (1H, m), 3.83-3.94 (1H, m), 4.04-4.12 (1H, m), 4.24 (2H, d, J=6.6 Hz), 4.40 (2H, d, J=5.7 Hz), 6.57 (1H, d, J=7.8 Hz), 6.61 (2H, s), 6.69 (1H, t, J=6.0 Hz), 6.80 (1H, d, J=8.5 Hz), 6.84 (1H, d, J=1.3 Hz), 7.02 (1H, d, J=1.3 Hz), 7.16 (1H, app t, J=8.0 Hz), 7.19 (1H, d, J=6.2 Hz), 7.34 (1H, d, J=8.3 Hz), 7.70-7.75 (2H, m), 8.19 (1H, d, J=2.4 Hz)

### Examples 4267 and 4412 (Enantiomers) N6-((2-((5,6,7,8-tetrahydroimidazo[1,2-a]pyridin-7-yl)methoxy)pyridin-4-yl)methyl)isoquinoline-1,6-diamine

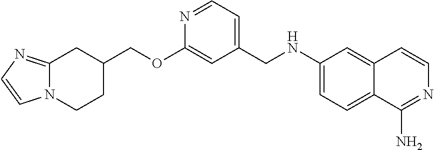 [SMILES: Nc1nccc2cc(NCc3ccnc(OCC4CCn5ccnc5C4)c3)ccc12]

### N1-(2,4-dimethoxybenzyl)-N5-((2-((5,6,7,8-tetrahydroimidazo[1,2-a]pyridin-7-yl)methoxy)pyridin-4-yl)methyl)isoquinoline-1,5-diamine

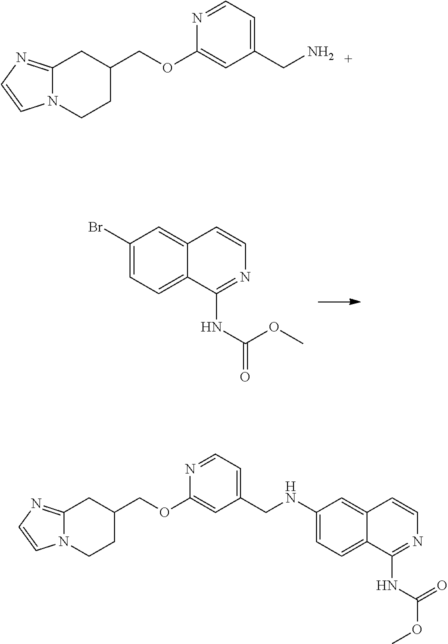 [SMILES: NCc1ccnc(OCC2CCn3ccnc3C2)c1 ; COC(=O)Nc1nccc2cc(Br)ccc12 ; COC(=O)Nc1nccc2cc(NCc3ccnc(OCC4CCn5ccnc5C4)c3)ccc12]

Using General Method 4, (2-((5,6,7,8-tetrahydroimidazo[1,2-a]pyridin-7-yl)methoxy)pyridin-4-yl)methanamine (108 mg, 0.42 mmol) was reacted with methyl (6-bromoisoquinolin-1-yl)carbamate (129 mg, 0.46 mmol) and NaOtBu (80 mg, 0.84 mmol) 1,4-dioxane (5 mL) at 60° C. for 3 h. After quenching the reaction mixture, the crude was purified by flash chromatography (Silica, 0-20% (0.7M NH₃ in MeOH in DCM) to afford the racemate as an off-white solid.

The racemate was purified by SFC reverse phase chiral HPLC on a Waters prep 15 with UV detection by DAD at 210-400 nm, 40° C., 120 bar on a LUX A2 10×250 mm, 5 um Column flow rate 15 mL/min-1 using 50% of MeOH. The samples were lyophilised to afford enantiomer 1 and enantiomer 2 as colourless solids. Absolute configuration assigned arbitrarily.

### Enantiomer 1

### Methyl (R*)-(6-(((2-((5,6,7,8-tetrahydroimidazo[1,2-a]pyridin-7-yl)methoxy)pyridin-4-yl)methyl)amino)isoquinolin-1-yl)carbamate (43 mg, 22% yield)

[M+H]⁺=459.0; 100% ee (diode array).

### Enantiomer 2

### Methyl (S*)-(6-(((2-((5,6,7,8-tetrahydroimidazo[1,2-a]pyridin-7-yl)methoxy)pyridin-4-yl)methyl)amino)isoquinolin-1-yl)carbamate

(43 mg, 22% yield)

[M+H]⁺=459.0; 100% ee (diode array).

### (S*)-N6-((2-((5,6,7,8-tetrahydroimidazo[1,2-a]pyridin-7-yl)methyl)isoquinoline-1,6-diamine (enantiomer 2, Example Number 4267)

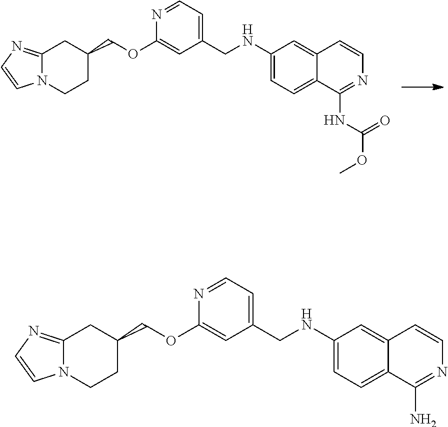 [SMILES: COC(=O)Nc1nccc2cc(NCc3ccnc(OC[C@H]4CCn5ccnc5C4)c3)ccc12 ; Nc1nccc2cc(NCc3ccnc(OC[C@H]4CCn5ccnc5C4)c3)ccc12]

Deprotection of methyl (S*)-(6-(((2-((5,6,7,8-tetrahydroimidazo[1,2-a]pyridin-7-yl)methoxy)pyridin-4-yl)methyl)amino)isoquinolin-1-yl)carbamate (43 mg, 0.094 mmol) was performed using General Method 14a, for 24 h. After quenching and elution through an SCX, the product was lyophilised to yield the product (30 mg, 80% yield) as a white fluffy solid.

[M+H]⁺=401.5

¹H NMR (500 MHz, DMSO-d6) δ 1.65-1.75 (1H, m), 2.07-2.15 (1H, m), 2.29-2.41 (1H, m), 2.43-2.47 (1H, m), 2.86-2.94 (1H, m), 3.82-3.91 (1H, m), 4.02-4.10 (1H, m), 4.24 (2H, d, J=6.6 Hz), 4.39 (2H, d, J=6.1 Hz), 6.31 (2H, s), 6.44 (1H, d, J=2.4 Hz), 6.53 (1H, d, J=5.8 Hz), 6.79 (1H, d, J=1.3 Hz), 6.81 (1H, s), 6.85 (1H, t, J=6.2 Hz), 6.88 (1H, dd, J=9.0, 2.4 Hz), 6.94-7.04 (2H, m), 7.54 (1H, d, J=5.8 Hz), 7.87 (1H, d, J=9.1 Hz), 8.08 (1H, d, J=5.3 Hz).

### (R*)-N6-((2-((5,6,7,8-tetrahydroimidazo[1,2-a]pyridin-4-yl)methyl)isoquinoline-1,6-diamine (enantiomer 1, Example Number 4412)

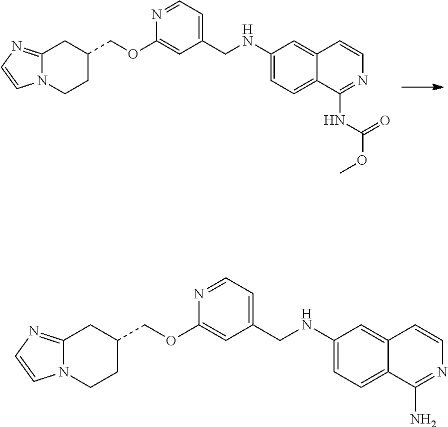 [SMILES: COC(=O)Nc1nccc2cc(NCc3ccnc(OC[C@@H]4CCn5ccnc5C4)c3)ccc12 ; Nc1nccc2cc(NCc3ccnc(OC[C@@H]4CCn5ccnc5C4)c3)ccc12]

Deprotection of methyl (R*)-(6-(((2-((5,6,7,8-tetrahydroimidazo[1,2-a]pyridin-7-yl)methoxy)pyridin-4-yl)methyl)amino)isoquinolin-1-yl)carbamate (43 mg, 0.094 mmol) was performed using General Method 14a for 24 h. After quenching and elution through an SCX, the product was lyophilised to yield the product (43 mg, 93% yield) as a white fluffy solid.

[M+H]⁺=401.5

¹H NMR (500 MHz, DMSO-d6) 1.63-1.79 (1H, m), 2.05-2.18 (1H, m), 2.30-2.39 (1H, m), 2.45-2.50 (1H, m), 2.85-2.96 (1H, m), 3.78-3.93 (1H, m), 4.01-4.13 (1H, m), 4.24 (2H, d, J=6.5 Hz), 4.39 (2H, d, J=6.0 Hz), 6.26-6.35 (2H, m), 6.44 (1H, d, J=2.3 Hz), 6.53 (1H, d, J=5.8 Hz), 6.78-6.93 (4H, m), 6.96-7.04 (2H, m), 7.54 (1H, d, J=5.8 Hz), 7.87 (1H, d, J=9.0 Hz), 8.08 (1H, d, J=5.3 Hz).

### Example Number 1033

### N6-((6-(((1-methylpiperidin-4-yl)methyl)amino)pyridin-3-yl)methyl)isoquinoline-1,6-diamine

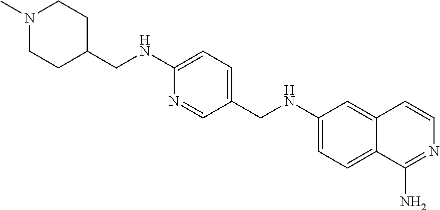 [SMILES: CN1CCC(CNc2ccc(CNc3ccc4c(N)nccc4c3)cn2)CC1]

### Methyl (6-(((6-((1-methylpiperidin-4-yl)methoxy)pyridin-3-yl)methyl)amino)isoquinolin-1-yl)carbamate

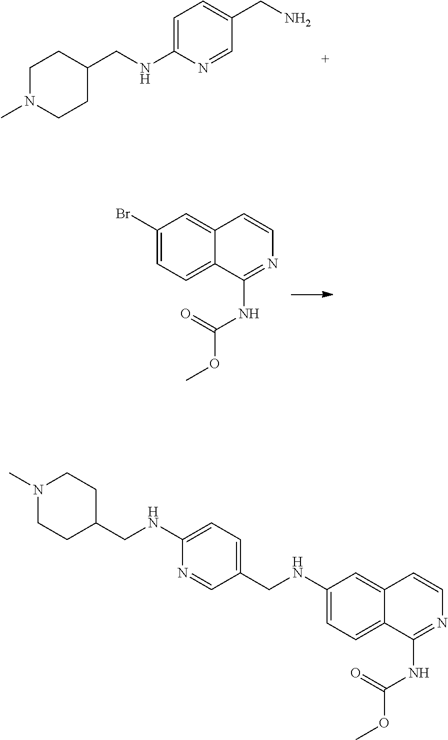 [SMILES: CN1CCC(CNc2ccc(CN)cn2)CC1 ; COC(=O)Nc1nccc2cc(Br)ccc12 ; COC(=O)Nc1nccc2cc(NCc3ccc(NCC4CCN(C)CC4)nc3)ccc12]

Following General Method 4, 5-(aminomethyl)-N-((1-methylpiperidin-4-yl)methyl)pyridin-2-amine (73 mg, 0.31 mmol) was reacted with methyl (6-bromoisoquinolin-1-yl)carbamate (90 mg, 0.32 mmol) and NaOtBu (60 mg, 0.62 mmol in THF (5 mL) at 60° C. for 2 h. After quenching the reaction mixture and concentrating, the crude product was purified by flash chromatography (Silica, 0-20% (0.7M NH₃ in MeOH) in DCM) to afford the product (120 mg, 86% yield) as an off-white solid.

[M+H]⁺=435.4

### N6-((6-(((1-methylpiperidin-4-yl)methyl)amino)pyridin-3-yl)methyl)isoquinoline-1,6-diamine

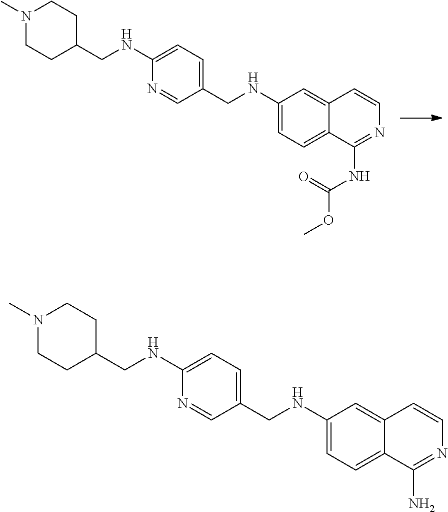 [SMILES: COC(=O)Nc1nccc2cc(NCc3ccc(NCC4CCN(C)CC4)nc3)ccc12 ; CN1CCC(CNc2ccc(CNc3ccc4c(N)nccc4c3)cn2)CC1]

Deprotection of methyl (6-(((6-(((1-methylpiperidin-4-yl)methyl)amino)pyridin-3-yl)methyl)amino)isoquinolin-1-yl)carbamate (120 mg, 0.249 mmol) was carried out using General Method 14a for 20 h. The crude product was purified by reverse phase flash chromatography (Silica, C18, 0-100% THF in 10 mM NH₄HCO₃) to afford the product (22 mg, 22% yield) as a pale yellow solid.

[M+H]⁺=377.2

¹H NMR (500 MHz, DMSO-d6) δ 1.10-1.20 (2H, m), 1.41-1.52 (1H, m), 1.61-1.69 (2H, m), 1.73-1.82 (2H, m), 2.12 (3H, s), 2.67-2.77 (2H, m), 3.06-3.12 (2H, m), 4.12 (2H, d, J=5.6 Hz), 6.34 (2H, s), 6.42-6.48 (2H, m), 6.50 (1H, t, J=5.6 Hz), 6.53 (1H, d, J=2.3 Hz), 6.58 (1H, d, J=5.8 Hz), 6.86 (1H, dd, J=9.1, 2.3 Hz), 7.37 (1H, dd, J=8.6, 2.4 Hz), 7.54 (1H, d, J=5.9 Hz), 7.84 (1H, d, J=9.1 Hz), 7.98 (1H, d, J=2.4 Hz).

### Example Number 4268

### 4-Chloro-N6-((2-((1-methylpiperidin-4-yl)methoxy)pyridin-4-yl)methyl)isoquinoline-1,6-diamine

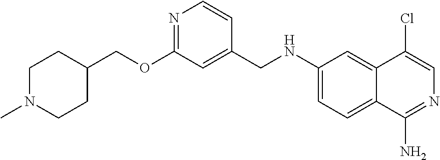 [SMILES: CN1CCC(COc2cc(CNc3ccc4c(N)ncc(Cl)c4c3)ccn2)CC1]

### Methyl (4-chloro-6-(((2-((1-methylpiperidin-4-yl)methoxy)pyridin-4-yl)methyl)amino)isoquinolin-1-yl)carbamate

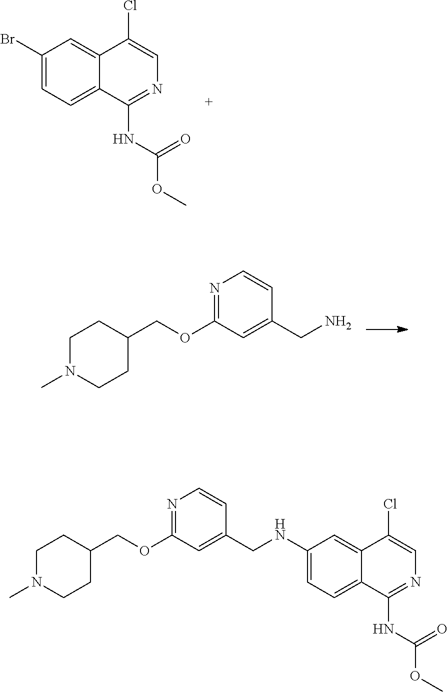

Using General Method 4, (2-((1-methylpiperidin-4-yl)methoxy)pyridin-4-yl)methanamine (23 mg, 0.10 mmol) was reacted with methyl (6-bromo-4-chloroisoquinolin-1-yl)carbamate (36 mg, 0.10 mmol), and NaOtBu (40 mg, 0.38 mmol) in THF (5 mL) at 40° C. and stirred for 5 h. After quenching the reaction mixture, the crude product was purified by flash chromatography (Silica, 0-20% (0.7M NH₃ in MeOH) in DCM) to afford the product (38 mg, 71% yield) as a yellow solid.

[M+H]⁺=470.2/472.2

### 4-Chloro-N6-((2-((1-methylpiperidin-4-yl)methoxy)pyridin-4-yl)methyl)isoquinoline-1,6-diamine, HCl

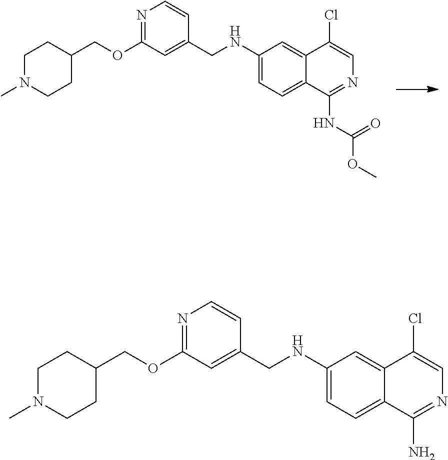

Deprotection of methyl (4-chloro-6-(((2-((1-methylpiperidin-4-yl)methoxy)pyridin-4-yl)methyl)amino)isoquinolin-1-yl)carbamate (35 mg, 0.06 mol) was performed using General Method 14a for 48 h. The reaction was cooled and concentrated. The crude product was purified by flash chromatography (Silica, 0-20% (0.7M NH₃ in MeOH) in DCM) to afford the product (19 mg, 65%) as a colourless solid.

[M+H]⁺=412.1

¹H NMR (500 MHz, DMSO-d6) 1.49-1.63 (2H, m), 1.85-1.92 (3H, m), 1.93-2.03 (1H, m), 2.69 (3H, s), 2.80-3.02 (3H, m), 4.13 (2H, d, J=6.4 Hz), 4.43 (2H, d, J=6.1 Hz), 6.66 (1H, d, J=2.3 Hz), 6.75-6.82 (3H, m), 6.98 (1H, dd, J=9.1, 2.4 Hz), 7.01 (1H, dd, J=5.3, 1.4 Hz), 7.35 (1H, t, J=6.2 Hz), 7.67 (1H, s), 7.99 (1H, d, J=9.1 Hz), 8.09 (1H, d, J=5.2 Hz), 10.20 (1H, s).

### Example Number 4270

### N6-((2-((3-methyl-3-azabicyclo[3.2.1]octan-8-yl)methoxy)pyridin-4-yl)methyl)isoquinoline-1,6-diamine

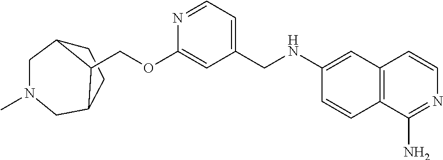 [SMILES: CN1CC2CCC(C1)C2COc1cc(CNc2ccc3c(N)nccc3c2)ccn1]

### Tert-butyl 8-(((4-cyanopyridin-2-yl)oxy)methyl)-3-azabicyclo[3.2.1]octane-3-carboxylate

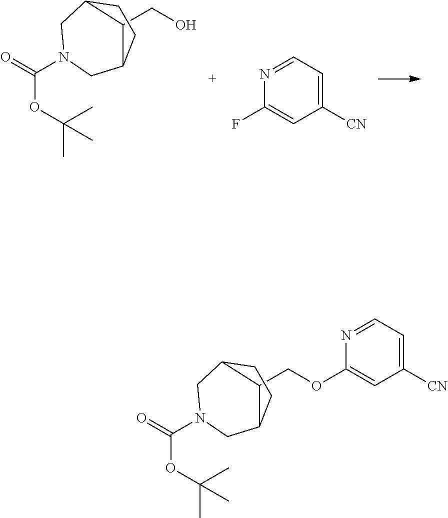 [SMILES: CC(C)(C)OC(=O)N1CC2CCC(C1)C2CO ; N#Cc1ccnc(F)c1 ; CC(C)(C)OC(=O)N1CC2CCC(C1)C2COc1cc(C#N)ccn1]

Using General Method 1b, tert-butyl-8-(hydroxymethyl)-3-azabicyclo[3.2.1]octane-3-carboxylate (300 mg, 1.24 mmol), was reacted with 2-fluoroisonicotinonitrile (152 mg, 1.24 mmol) for 7 days. The reaction was filtered and the filtrate was purified by flash chromatography (Silica, 0-50% EtOAc in Isohexane) to afford the product (355 mg, 81% yield) as a colourless crystalline solid.

[M+Na]⁺=366.1

¹H NMR (500 MHz, DMSO-d6) δ 1.39 (9H, s), 1.42-1.53 (2H, m), 1.66-1.77 (2H, m), 2.05-2.12 (1H, m), 2.13-2.23 (2H, m), 3.02 (1H, d, J=12.9 Hz), 3.15 (1H, d, J=13.0 Hz), 3.48 (1H, d, J=13.2 Hz), 3.54 (1H, d, J=13.0 Hz), 4.64 (2H, d, J=7.6 Hz), 7.37-7.43 (2H, m), 8.41 (1H, dd, J=5.1, 0.9 Hz) ppm.

### 2-((3-Methyl-3-azabicyclo[3.2.1]octan-8-yl)methoxy)isonicotinonitrile

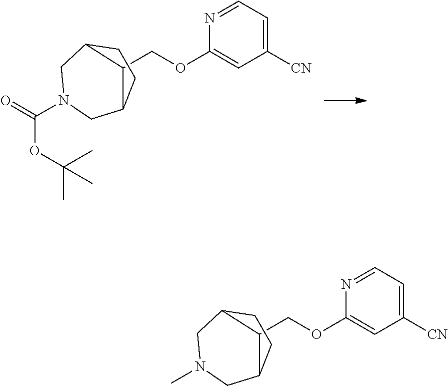 [SMILES: CC(C)(C)OC(=O)N1CC2CCC(C1)C2COc1cc(C#N)ccn1 ; CN1CC2CCC(C1)C2COc1cc(C#N)ccn1]

Tert-butyl 8-(((4-cyanopyridin-2-yl)oxy)methyl)-3-azabicyclo[3.2.1]octane-3-carboxylate (350 mg, 1.02 mmol) was reacted following General Method 10 for 2 h. The product was isolated (205 mg, 77% yield) as a colourless solid.

[M+H]⁺=258.1

### (2-((3-Methyl-3-azabicyclo[3.2.1]octan-8-yl)methoxy)pyridin-4-yl)methanamine

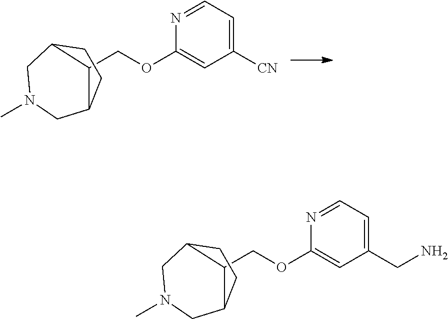 [SMILES: CN1CC2CCC(C1)C2COc1cc(C#N)ccn1 ; CN1CC2CCC(C1)C2COc1cc(CN)ccn1]

Reduction of the nitrile, 2-((3-Methyl-3-azabicyclo[3.2.1]octan-8-yl)methoxy)isonicotinonitrile (205 mg, 0.797 mmol) was performed following General Method 3a, over 3 h using Raney Ni. The reaction was concentrated to afford the product (190 mg, 85% yield) as a clear, colourless oil.

[M+H]⁺=262.2

¹H NMR (500 MHz, DMSO-d6) 1.58-1.72 (4H, m), 1.88-2.02 (3H, m), 2.10-2.16 (2H, m), 2.16 (3H, s), 2.31-2.36 (2H, m), 2.40 (2H, dd, J=11.1, 3.6 Hz), 3.68 (2H, s), 4.53 (2H, d, J=7.5 Hz), 6.78 (1H, s), 6.91 (1H, dd, J=5.3, 1.4 Hz), 8.04 (1H, d, J=5.3 Hz) ppm.

### Methyl (6-(((2-((3-methyl-3-azabicyclo[3.2.1]octan-8-yl)methoxy)pyridin-4-yl)methyl)amino)isoquinolin-1-yl)carbamate

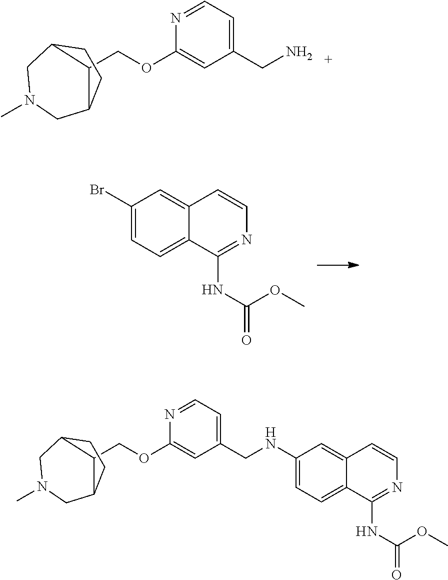

Following General Method 4, (2-((3-methyl-3-azabicyclo[3.2.1]octan-8-yl)methoxy)pyridin-4-yl)methanamine (90 mg, 0.34 mmol) was reacted with methyl (6-bromoisoquinolin-1-yl)carbamate (97 mg, 0.34 mmol), and NaOtBu (66 mg, 0.69 mmol) in THF (6 mL) at 60° C. for 3 h. After quenching the reaction, the crude product was purified by flash chromatography (Silica, 0-20% (0.7M NH₃ in MeOH) in DCM) to afford the product (99 mg, 53% yield) as a yellow gum.

[M+H]⁺=462.2

¹H NMR (500 MHz, DMSO-d6) δ 1.58-1.70 (4H, m), 1.91-1.98 (1H, m), 2.07-2.12 (2H, m), 2.15 (3H, s), 2.28-2.35 (2H, m), 2.35-2.45 (2H, m), 3.65 (3H, s), 4.42 (2H, d, J=6.2 Hz), 4.52 (2H, d, J=7.5 Hz), 6.52-6.63 (1H, m), 6.76 (1H, s), 6.95-6.99 (1H, m), 7.08 (1H, d, J=9.0 Hz), 7.13-7.26 (1H, m), 7.29-7.47 (1H, m), 7.76 (1H, d, J=9.1 Hz), 7.91-7.99 (1H, m), 8.10 (1H, dd, J=5.3, 0.7 Hz), 9.72 (1H, s) ppm.

### N6-((2-((3-methyl-3-azabicyclo[3.2.1]octan-8-yl)methoxy)pyridin-4-yl)methyl)isoquinoline-1,6-diamine

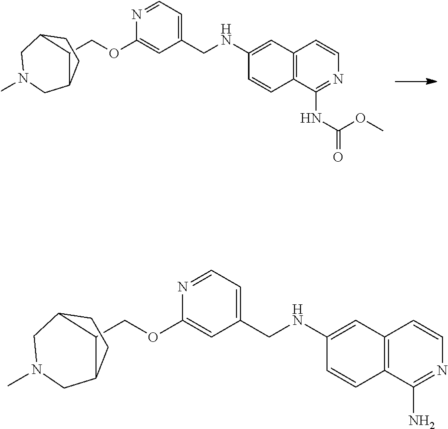

Deprotection of methyl (6-(((2-((3-methyl-3-azabicyclo[3.2.1]octan-8-yl)methoxy)pyridin-4-yl)methyl)amino)isoquinolin-1-yl)carbamate (95 mg, 0.21 mmol) was carried out using General Method 14a for 20 h. The crude product was purified by flash chromatography (Silica, 0-20% (0.7M NH₃ in MeOH) in DCM) to afford the product (9.0 mg, 10% yield) as a colourless solid.

[M+H]⁺=404.2

¹H NMR (DMSO, 500 MHz) δ 1.55-1.69 (4H, m), 1.92-1.99 (1H, m), 2.08-2.12 (2H, m), 2.14 (3H, s), 2.27-2.33 (2H, m), 2.38 (2H, dd, J=11.2, 3.6 Hz), 4.37 (2H, d, J=6.2 Hz), 4.51 (2H, d, J=7.5 Hz), 6.32 (2H, s), 6.43 (1H, d, J=2.4 Hz), 6.53 (1H, d, J=5.8 Hz), 6.75 (1H, s), 6.83 (1H, t, J=6.3 Hz), 6.88 (1H,dd, J=9.0, 2.4 Hz), 6.97 (1H, dd, J=5.3, 1.4 Hz), 7.54 (1H, d, J=5.8 Hz), 7.87 (1H, d, J=9.1 Hz), 8.09 (1H, d, J=5.3 Hz) ppm.

### Example Number 4275

### 1-(5-(((4-(((1-aminoisoquinolin-6-yl)amino)methyl)pyridin-2-yl)oxy)methyl)-2-azabicyclo[2.2.1]heptan-2-yl)ethan-1-one

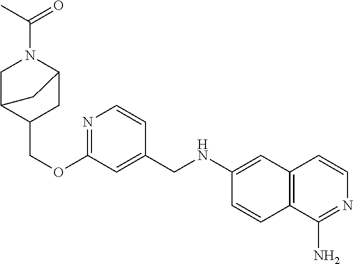 [SMILES: CC(=O)N1CC2CC1CC2COc1cc(CNc2ccc3c(N)nccc3c2)ccn1]

### Methyl (6-(((2-((2-acetyl-2-azabicyclo[2.2.1]heptan-5-yl)methoxy)pyridin-4-yl)methyl)amino)isoquinolin-1-yl)carbamate

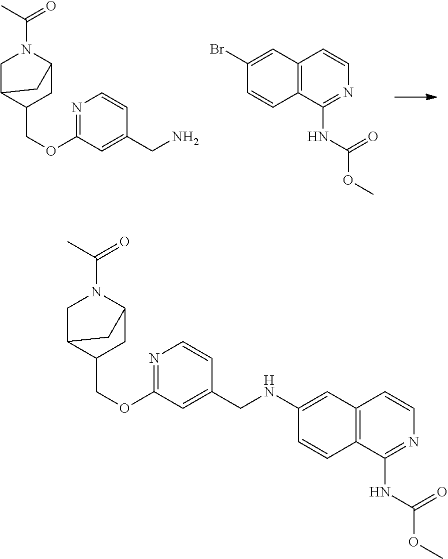 [SMILES: CC(=O)N1CC2CC1CC2COc1cc(CN)ccn1 ; COC(=O)Nc1nccc2cc(Br)ccc12 ; COC(=O)Nc1nccc2cc(NCc3ccnc(OCC4CC5CC4CN5C(C)=O)c3)ccc12]

Following General Method 4, 1-(5-(((4-(aminomethyl)pyridin-2-yl)oxy)methyl)-2-azabicyclo[2.2.1]heptan-2-yl)ethan-1-one (125 mg, 0.45 mmol) was reacted with methyl (6-bromoisoquinolin-1-yl)carbamate (128 mg, 0.45 mmol) and NaOtBu (26 mg, 0.27 mmol) in THF (6 mL) at 60° C. for 2 h. After quenching the reaction, the crude product was purified by flash chromatography (Silica, 0-15% (0.7M NH₃ in MeOH) in DCM) to afford the product (148 mg, 65% yield) as a colourless glass.

[M+H]⁺=476.2

### 1-(5-(((4-(((1-Aminoisoquinolin-6-yl)amino)methyl)pyridin-2-yl)oxy)methyl)-2-azabicyclo[2.2.1]heptan-2-yl)ethan-1-one

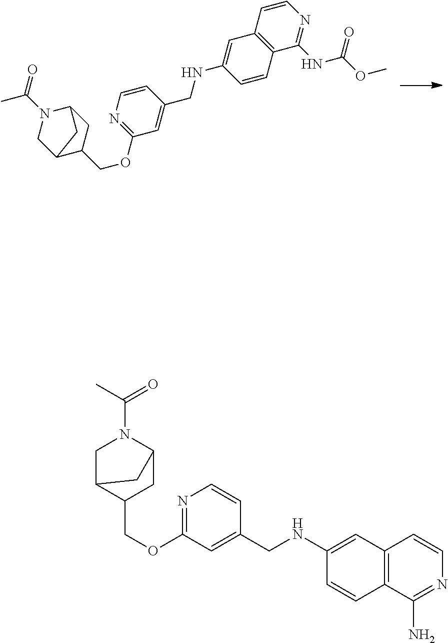 [SMILES: COC(=O)Nc1nccc2cc(NCc3ccnc(OCC4CC5CC4CN5C(C)=O)c3)ccc12 ; CC(=O)N1CC2CC1CC2COc1cc(CNc2ccc3c(N)nccc3c2)ccn1]

Deprotection of methyl (6-(((2-((2-acetyl-2-azabicyclo[2.2.1]heptan-5-yl)methoxy)pyridin-4-yl)methyl)amino)isoquinolin-1-yl)carbamate (148 mg, 0.31 mmol) was performed using General Method 14a for 16 h. After quenching the reaction mixture, the crude product was purified by flash chromatography (Silica, 0-100% (2% NH₃ in EtOAc/IPA (3:1)) in Hexane). Lyophilisation afforded the product (91 mg, 68% yield) as a colourless solid.

[M+H]⁺=418.2

¹H NMR (500 MHz, DMSO-d6) 1.05-1.11 (1H, m, minor), 1.15-1.20 (1H, m, major), 1.50-1.55 (1H, m, minor), 1.58-1.63 (1H, m), 1.69-1.73 (1H, m, major), 1.82 (3H, s, minor), 1.92 (3H, s, major), 1.78-1.96 (1H, m), 2.40-2.49 (1H, m), 2.54-2.62 (1H, m), 2.99-3.03 (1H, m, minor), 3.23-3.28 (2×H, m, major), 3.34-3.38 (1H, m, minor), 4.03-4.17 (1H, m and 1H, m, minor), 4.20-4.26(1H, m, major), 4.32-4.36 (1H, m), 4.38 (2H, d, J=6.4 Hz), 6.31 (2H, s), 6.42-6.43 (1H, m), 6.53 (1H, dd, J=5.9, 2.3 Hz), 6.75 (1H, s), 6.82-6.86 (1H, m), 6.88 (1H, dd, J=9.0, 2.3 Hz), 6.98 (1H, dd, J=5.3, 1.4 Hz), 7.54 (1H, d, J=5.8 Hz), 7.87 (1H, d, J=9.0 Hz), 8.06-8.09 (1H, m)

### Example Number 4274

### 1-(5-(((4-(((1-Aminoisoquinolin-5-yl)amino)methyl)pyridin-2-yl)oxy)methyl)-2-azabicyclo[2.2.1]heptan-2-yl)ethan-1-one

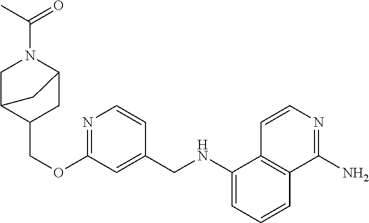 [SMILES: CC(=O)N1CC2CC1CC2COc1cc(CNc2cccc3c(N)nccc23)ccn1]

### Methyl (5-(((2-((2-acetyl-2-azabicyclo[2.2.1]heptan-5-yl)methoxy)pyridin-4-yl)methyl)amino)isoquinolin-1-yl)carbamate

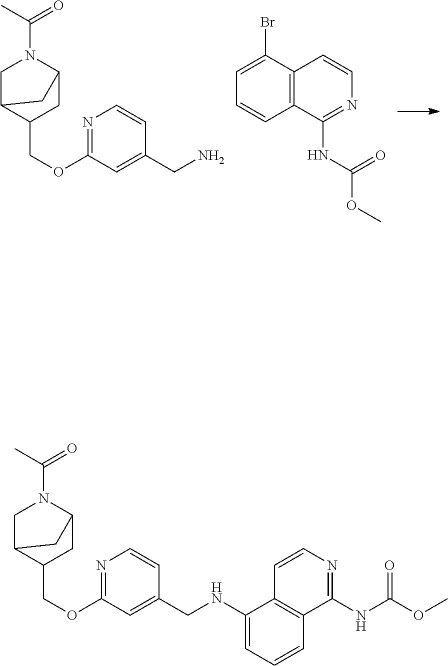 [SMILES: CC(=O)N1CC2CC1CC2COc1cc(CN)ccn1 ; COC(=O)Nc1nccc2c(Br)cccc12 ; COC(=O)Nc1nccc2c(NCc3ccnc(OCC4CC5CC4CN5C(C)=O)c3)cccc12]

Following General Method 4, 1-(5-(((4-(aminomethyl)pyridin-2-yl)oxy)methyl)-2-azabicyclo[2.2.1]heptan-2-yl)ethan-1-one (125 mg, 0.45 mmol) was reacted with methyl (5-bromoisoquinolin-1-yl)carbamate (128 mg, 0.45 mmol) and NaOtBu (90 mg, 0.94 mmol) in THF (6 mL) at 60° C. for 5 h. After quenching the reaction, the crude product was purified by flash chromatography (Silica, 0-15% (0.7M NH₃ in MeOH) in DCM). Lyophilisation afforded the product (140 mg, 62% yield) as a colourless solid.

[M+H]⁺=476.2

### 1-(5-(((4-(((1-Aminoisoquinolin-5-yl)amino)methyl)pyridin-2-yl)oxy)methyl)-2-azabicyclo[2.2.1]heptan-2-yl)ethan-1-one

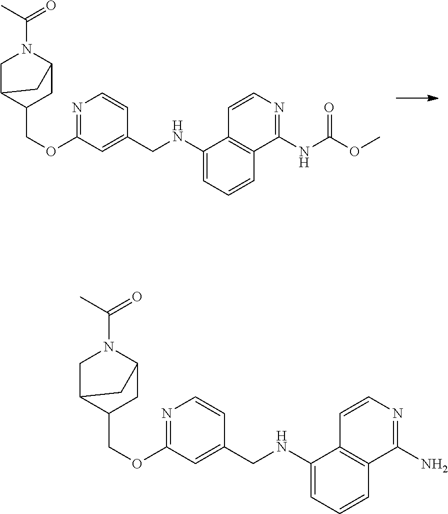 [SMILES: COC(=O)Nc1nccc2c(NCc3ccnc(OCC4CC5CC4CN5C(C)=O)c3)cccc12 ; CC(=O)N1CC2CC1CC2COc1cc(CNc2cccc3c(N)nccc23)ccn1]

Deprotection of methyl (5-(((2-((2-acetyl-2-azabicyclo[2.2.1]heptan-5-yl)methoxy)pyridin-4-yl)methyl)amino)isoquinolin-1-yl)carbamate (140 mg, 0.29 mmol) was performed using General Method 14a for 18 h. After quenching the reaction mixture, the crude product was purified by flash chromatography (Silica, 0-15% (0.7M NH₃ in MeOH) in DCM). Lyophilisation afforded the product (70 mg, 56% yield) as a colourless solid.

[M+H]⁺=418.2

¹H NMR (500 MHz, DMSO-d6) 1.04-1.09 (1H, m, minor), 1.13-1.19 (1H, m, major), 1.49-1.54 (1H, m, minor), 1.56-1.63 (1H, m), 1.68-1.73 (1H, m, major), 1.81 (3H, s, minor), 1.92 (3H, s, major), 1.78-1.96 (1H, m), 2.40-2.48 (1H, m), 2.52-2.61 (1H, m), 2.98-3.03 (1H, m, major), 3.22-3.27 (1H, m), 3.29-3.37(1H, m, minor), 4.02-4.24 (2H, m), 4.30-4.36 (1H, m), 4.45 (2H, d, J=6.0 Hz), 6.38 (1H, d, J=7.7 Hz), 6.53 (2H, s), 6.72 (1H, s), 6.78-6.83 (1H, m), 6.98 (1H, dd, J=5.2, 1.4 Hz), 7.09-7.14 (1H, m), 7.20 (1H, d, J=6.1 Hz), 7.34 (1H, d, J=8.3 Hz), 7.77 (1H, d, J=6.0 Hz), 8.06 (1H, t, J=5.1 Hz)

### Example Number 4277

### N6-((2-((2-methyl-2-azabicyclo[2.2.1]heptan-5-yl)methoxy)pyridin-4-yl)methyl)isoquinoline-1,6-diamine

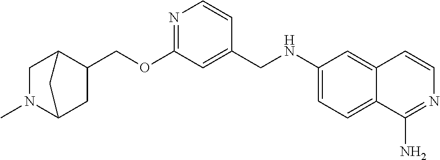 [SMILES: CN1CC2CC1CC2COc1cc(CNc2ccc3c(N)nccc3c2)ccn1]

### 2-((2-Methyl-2-azabicyclo[2.2.1]heptan-5-yl)methoxy)isonicotinonitrile

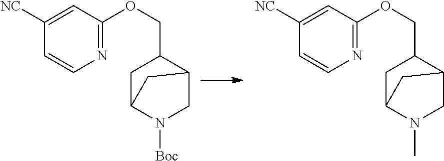 [SMILES: CC(C)(C)OC(=O)N1CC2CC1CC2COc1cc(C#N)ccn1 ; CN1CC2CC1CC2COc1cc(C#N)ccn1]

Tert-butyl 5-(((4-cyanopyridin-2-yl)oxy)methyl)-2-azabicyclo[2.2.1]heptane-2-carboxylate (500 mg, 1.52 mmol) was reacted following General Method 10 for 2 h. The crude product was purified by flash chromatography (Silica, 0-20% (0.7M NH₃ in MeOH) in DCM) to afford the product (272 mg, 58% yield) as a clear colourless oil.

[M+H]⁺=244.1

¹H NMR (500 MHz, DMSO-d6) 1.14-1.21 (1H, m), 1.29-1.36 (1H, m), 1.59-1.69 (2H, m), 2.21 (3H, s), 2.26-2.34 (2H, m), 2.36-2.41 (1H, m), 2.57-2.66 (1H, m), 2.93-2.99 (1H, m), 4.20-4.28 (1H, m), 4.34-4.42 (1H, m), 7.33-7.42 (2H, m), 8.40 (1H, d, J=5.4 Hz).

### (2-((2-Methyl-2-azabicyclo[2.2.1]heptan-5-yl)methoxy)pyridin-4-yl)methanamine

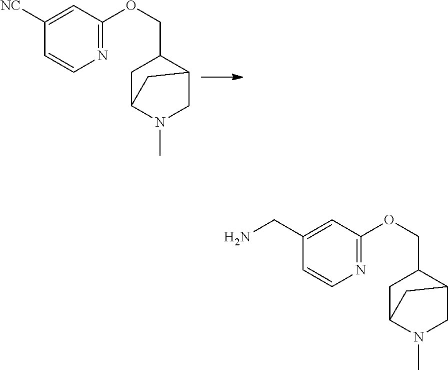 [SMILES: CN1CC2CC1CC2COc1cc(C#N)ccn1 ; CN1CC2CC1CC2COc1cc(CN)ccn1]

Reduction of the nitrile, 2-((2-methyl-2-azabicyclo[2.2.1]heptan-5-yl)methoxy)isonicotinonitrile (270 mg, 1.11 mmol) was carried out according to General Method 3a over 2 h using Raney Ni. The reaction was concentrated to afford the product (280 mg, 97% yield) as a clear, colourless oil.

[M+H]⁺=248.1

¹H NMR (500 MHz, DMSO-d6) 1.15-1.23 (1H, m), 1.31-1.38 (1H, m), 1.59-1.72 (2H, m), 2.23 (3H, s), 2.26-2.40 (3H, m), 2.62-2.69 (1H, m), 2.95-3.03 (1H, m), 3.06-3.45 (2H, m), 3.68 (2H, s), 4.17 (1H, dd, J=10.8, 9.3 Hz), 4.32 (1H, dd, J=10.7, 6.7 Hz), 6.76 (1H, s), 6.91 (1H, d, J=5.2 Hz), 8.02 (1H, d, J=5.2 Hz).

### Methyl (6-(((2-((2-methyl-2-azabicyclo[2.2.1]heptan-5-yl)methoxy)pyridin-4-yl)methyl)amino)isoquinolin-1-yl)carbamate

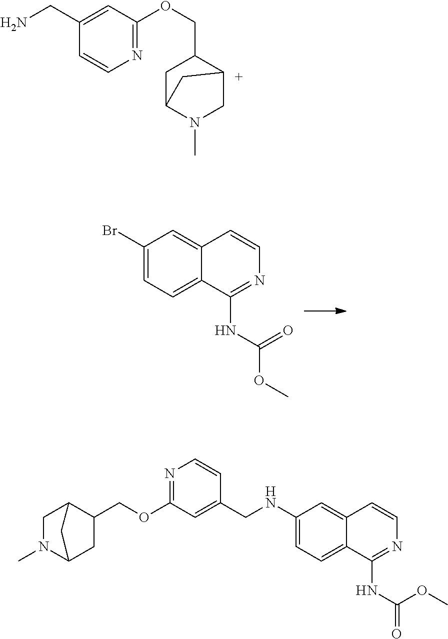

Following General Method 4, (2-((2-methyl-2-azabicyclo[2.2.1]heptan-5-yl)methoxy)pyridin-4-yl)methanamine (130 mg, 0.53 mmol) was reacted with methyl (6-bromoisoquinolin-1-yl)carbamate (148 mg, 0.53 mmol) and NaOtBu (101 mg, 1.05 mmol) in THF (6 mL) at 60° C. for 2 h. After quenching, the crude product was purified by flash chromatography (Silica, 0-20% (0.7M NH₃ in MeOH) in DCM) to afford the product (189 mg, 77% yield) as a colourless oil.

[M+H]⁺=448.5

### N6-((2-((2-methyl-2-azabicyclo[2.2.1]heptan-5-yl)methoxy)pyridin-4-yl)methyl)isoquinoline-1,6-diamine

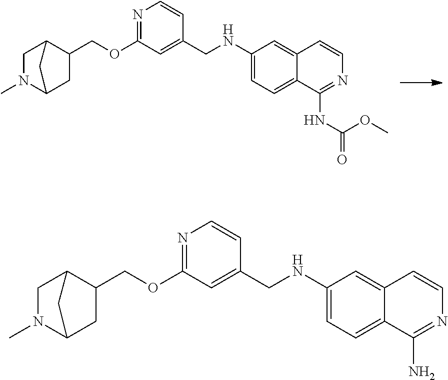

Deprotection of methyl (6-(((2-((2-methyl-2-azabicyclo[2.2.1]heptan-5-yl)methoxy)pyridin-4-yl)methyl)amino)isoquinolin-1-yl)carbamate (180 mg, 0.40 mmol) was carried out using General Method 14a over 24 h. After quenching and elution through an SCX, the crude product was purified by flash chromatography (Silica, 0-100% (10% NH₃ in MeOH) in DCM). Lyophilisation afforded the product (74 mg, 45% yield) as a pale yellow solid.

[M+H]⁺=390.2

¹H NMR (500 MHz, DMSO-d6) 1.11-1.18 (1H, m), 1.27-1.32 (1H, m), 1.56-1.67 (2H, m), 2.19 (3H, s), 2.22-2.32 (2H, m), 2.32-2.36 (1H, m), 2.56-2.61 (1H, m), 2.91-2.96 (1H, m), 4.11-4.19 (1H, m), 4.30 (1H, dd, J=10.8, 6.7 Hz), 4.37 (2H, d, J=6.2 Hz), 6.32 (2H, s), 6.43 (1H, d, J=2.4 Hz), 6.53 (1H, dd, J=5.9, 0.7 Hz), 6.72-6.76 (1H, m), 6.83 (1H, t, J=6.3 Hz), 6.88 (1H, dd, J=9.0, 2.4 Hz), 6.97 (1H, dd, J=5.3, 1.4 Hz), 7.54 (1H, d, J=5.8 Hz), 7.87 (1H, d, J=9.1 Hz), 8.05-8.08 (1H, m).

### Example Number 4285

### 4-Chloro-N6-((2-(((1-methylpiperidin-4-yl)methyl)amino)pyridin-4-yl)methyl)isoquinoline-1,6-diamine

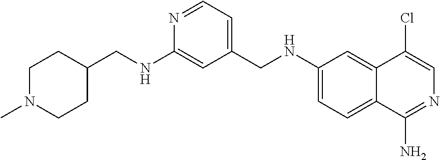 [SMILES: CN1CCC(CNc2cc(CNc3ccc4c(N)ncc(Cl)c4c3)ccn2)CC1]

### Methyl N-[4-chloro-6-[[2-[(1-methyl-4-piperidyl)methylamino]-4-pyridyl]methylamino]-1-isoquinolyl]carbamate

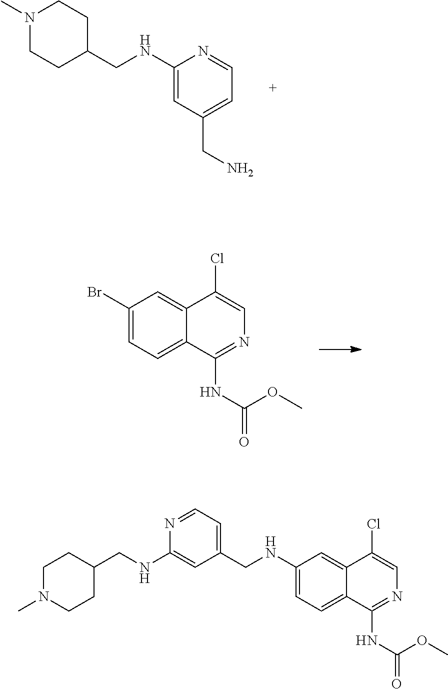

Following General Method 4, methyl N-(6-bromo-4-chloro-1-isoquinolyl)carbamate (44 mg, 0.13 mmol) was reacted with 4-(aminomethyl)-N-[(1-methyl-4-piperidyl)methyl]pyridin-2-amine (30 mg, 0.13 mmol) and NaOtBu (168 mg, 0.51 mmol) in THF (5 mL) at 40° C. for 9 h. The reaction was cooled to rt filtered through Celite®, washing with EtOAc (50 mL), DCM (50 mL) and MeOH (50 mL). The filtrate was concentrated to afford the product (24 mg, 40% yield) as brown oil.

[M+H]⁺=469.1

### 4-Chloro-N6-[[2-[(1-methyl-4-piperidyl)methylamino]-4-pyridyl]methyl]isoquinoline-1,6-diamine

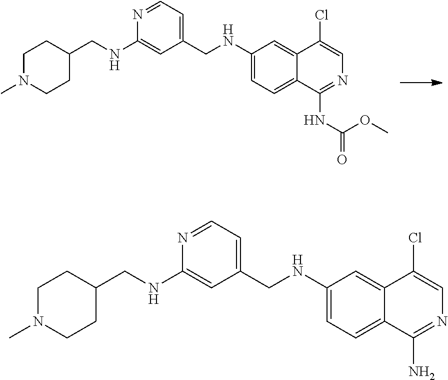

Deprotection of methyl N-[4-chloro-6-[[2-[(1-methyl-4-piperidyl)methylamino]-4-pyridyl]methylamino]-1-isoquinolyl]carbamate (20 mg, 0.04 mmol) was carried out using General Method 14b for 12 h. After quenching and elution through an SCX, the crude product was purified via automated prep HPLC (Mass directed 2-60% over 20 min in basic mobile phase). Lyophilisation afforded the product (5 mg, 24% yield) as an off-white solid.

[M+H]⁺=411.1

¹H NMR (DMSO, 400 MHz) δ 1.03-1.17 (2H, m), 1.35-1.46 (1H, m), 1.59 (2H, d, J=10.7 Hz), 1.70 (2H, td, J=11.5, 2.6 Hz), 2.09 (3H, s), 2.63-2.72 (2H, m), 3.05 (2H, d, J=6.3 Hz), 4.25 (2H, d, J=6.0 Hz), 6.42 (1H, s), 6.44 (1H, dd, J=5.2, 1.6 Hz), 6.49 (1H, t, J=5.8 Hz), 6.54 (2H, s), 6.64 (1H, d, J=2.4 Hz), 6.91 (1H, dd, J=9.0, 2.4 Hz), 7.12 (1H, d, J=6.1 Hz), 7.64 (1H, s), 7.86 (1H, d, J=5.3 Hz), 7.92 (1H, d, J=9.0 Hz)

### Example Number 2208

### N5-[[2-fluoro-4-[2-(1-methylimidazol-2-yl)ethoxymethyl]phenyl]methyl]isoquinoline-1,5-diamine

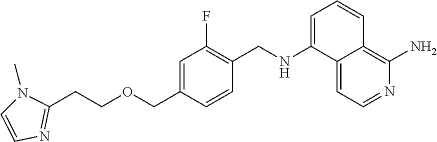 [SMILES: Cn1ccnc1CCOCc1ccc(CNc2cccc3c(N)nccc23)c(F)c1]

### Tert-butyl N-[[2-fluoro-4-(hydroxymethyl)phenyl]methyl]carbamate

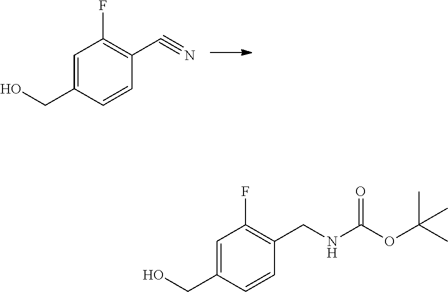 [SMILES: N#Cc1ccc(CO)cc1F ; CC(C)(C)OC(=O)NCc1ccc(CO)cc1F]

Following General Method 3c, 2-fluoro-4-(hydroxymethyl)benzonitrile (1.9 g, 12.57 mmol) was reduced over 72 h. The reaction mixture was filtered through Celite®, concentrated and redissolved in THF (100 mL). Boc₂O (2.7 g, 12.57 mmol) was added and the reaction stirred at 60° C. for 18 h. The reaction was concentrated and the crude product was purified by flash chromatography (Silica, 0-8% MeOH in DCM) to afford product (1.9 g, 59% yield) as an off white solid.

¹H NMR (DMSO, 400 MHz) δ 1.39 (9H, s), 4.14 (2H, d, J=6.1 Hz), 4.47 (2H, d, J=5.6 Hz), 5.26 (1H, t, J=5.8 Hz), 7.08 (2H, t, J=10.3 Hz), 7.24 (1H, t, J=7.8 Hz), 7.35 (1H, t, J=6.2 Hz)

### Tert-butyl N-[[4-(chloromethyl)-2-fluoro-phenyl]methyl]carbamate

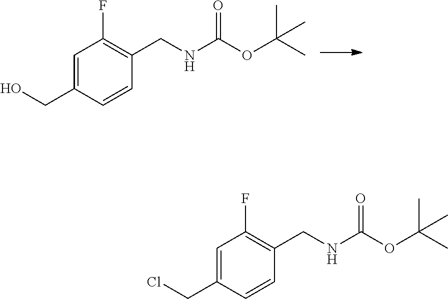 [SMILES: CC(C)(C)OC(=O)NCc1ccc(CO)cc1F ; CC(C)(C)OC(=O)NCc1ccc(CCl)cc1F]

Chlorination of tert-butyl N-[[2-fluoro-4-(hydroxymethyl)phenyl]methyl]carbamate (900 mg, 3.33 mmol) was carried out using General Method 6a. The crude product was purified by flash chromatography (Silica, 20-80% EtOAc in Pet. Ether 60-80) to afford the product (705 mg, 77% yield) as an off white solid.

[M-tBu+H]⁺=218.0

### Tert-butyl N-[[2-fluoro-4-[2-(1-methylimidazol-2-yl)ethoxymethyl]phenyl]methyl]carbamate

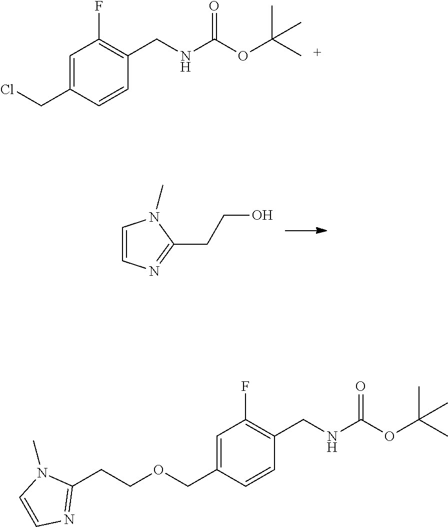

Following General Method 5a, 2-(1-methyl-1H-imidazol-2-yl)ethan-1-ol (55 mg, 0.44 mmol) was reacted with (tert-butyl N-[[4-(chloromethyl)-2-fluoro-phenyl]methyl]carbamate (100 mg, 0.37 mmol) for 3 h. The crude product was purified by flash chromatography (Silica, 0-12% MeOH in DCM) to afford the product (52 mg, 39% yield) as an off white solid.

[M+H]⁺=364.1

### [2-Fluoro-4-[2-(1-methylimidazol-2-yl)ethoxymethyl]phenyl]methanamine

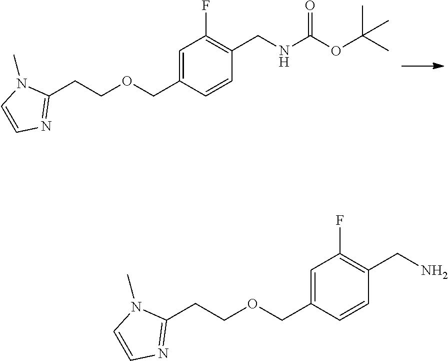 [SMILES: Cn1ccnc1CCOCc1ccc(CNC(=O)OC(C)(C)C)c(F)c1 ; Cn1ccnc1CCOCc1ccc(CN)c(F)c1]

Boc deprotection of tert-butyl N-[[2-fluoro-4-[2-(1-methylimidazol-2-yl)ethoxymethyl]phenyl]methyl]carbamate (52 mg, 0.14 mmol) was carried out following General Method 7a, at rt for 45 min. The reaction mixture was concentrated. The crude was taken up in MeOH (2 mL) and passed through bicarbonate resin, washing with MeOH (10 mL). The filtrate was concentrated and triturated with Et₂O (2×10 mL) to afford the product (37 mg, 98% yield) as an off white solid.

[M+H]⁺=264.0

### N1-[(2,4-dimethoxyphenyl)methyl]-N5-[[2-fluoro-4-[2-(1-methylimidazol-2-yl)ethoxymethyl]phenyl]methyl]isoquinoline-1,5-diamine

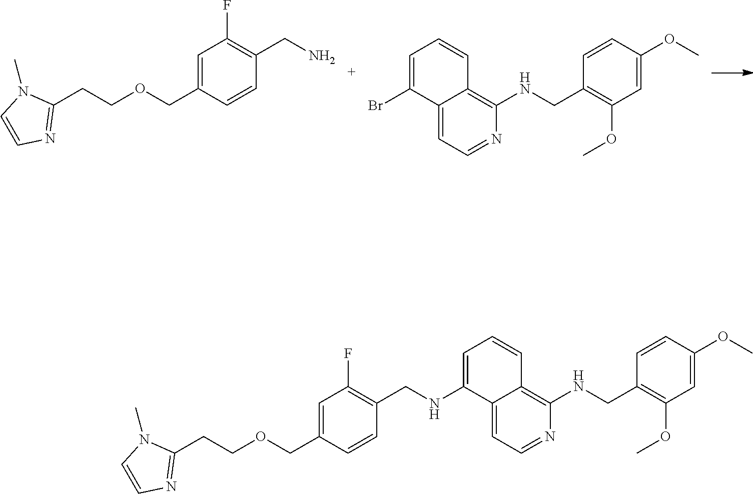 [SMILES: Cn1ccnc1CCOCc1ccc(CN)c(F)c1 ; COc1ccc(CNc2nccc3c(Br)cccc23)c(OC)c1 ; COc1ccc(CNc2nccc3c(NCc4ccc(COCCc5nccn5C)cc4F)cccc23)c(OC)c1]

Following General Method 4, 5-bromo-N-[(2,4-dimethoxyphenyl)methyl]isoquinolin-1-amine (35 mg, 0.09 mmol) was reacted with [2-fluoro-4-[2-(1-methylimidazol-2-yl)ethoxymethyl]phenyl]methanamine (25 mg, 0.09 mmol) and NaOtBu (62 mg, 0.19 mmol) in 1,4-dioxane (5 mL) at 60° C. for 6 h. After quenching and filtering through Celite®, the crude product was purified by flash chromatography (Silica, 0-24% (10% NH₃ in MeOH) in DCM) to the product (21 mg, 40% yield) as a yellow gum.

[M+H]⁺=556.3

### N3-[[2-fluoro-4-[2-(1-methylimidazol-2-yl)ethoxymethyl]phenyl]methyl]isoquinoline-1,5-diamine

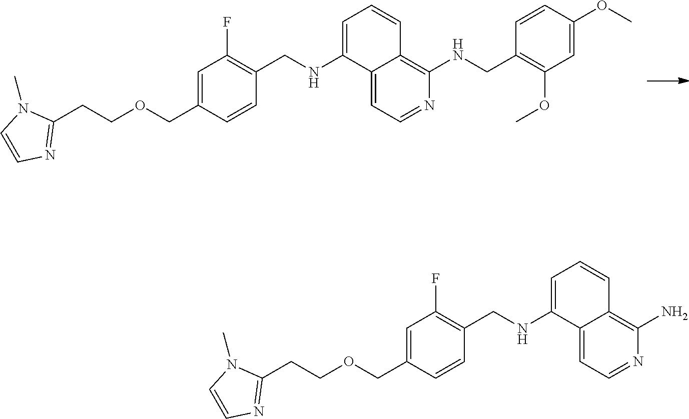 [SMILES: COc1ccc(CNc2nccc3c(NCc4ccc(COCCc5nccn5C)cc4F)cccc23)c(OC)c1 ; Cn1ccnc1CCOCc1ccc(CNc2cccc3c(N)nccc23)c(F)c1]

Using General Method 12, N1-[(2,4-dimethoxyphenyl)methyl]-N5-[[2-fluoro-4-[2-(1-methylimidazol-2-yl)ethoxymethyl]phenyl]methyl]isoquinoline-1,5-diamine (25 mg, 0.04 mmol) was deprotected in TFA (1 mL, 12.98 mmol) was heated to 50° C. for 25 min. The crude product was purified via automated prep HPLC (Mass directed 2-60% over 20 min in basic mobile phase). Lyophilisation afforded the product N5-[[2-fluoro-4-[2-(1-methylimidazol-2-yl)ethoxymethyl]phenyl]methyl]isoquinoline-1,5-diamine (1 mg, 5% yield) as an off-white solid.

[M+H]⁺=406.1

¹H NMR (CDCl₃, 400 MHz) δ 2.99 (2H, t, J=6.8 Hz), 3.59 (3H, s), 3.87 (2H, t, J=6.9 Hz), 4.50 (2H, s), 4.53 (2H, d, J=5.2 Hz), 4.68 (1H, s), 5.14 (2H, s), 6.73 (1H, d, J=7.7 Hz), 6.79 (1H, d, J=1.4 Hz), 6.93 (1H, d, J=1.4 Hz), 6.96-7.06 (3H, m), 7.15 (1H, d, J=8.3 Hz), 7.33 (2H, t, J=7.9 Hz), 7.93 (1H, d, J=6.1 Hz)

### Example Number 2183

### N5-[[2-fluoro-4-(2-morpholinoethyl)phenyl]methyl]isoquinoline-1,5-diamine

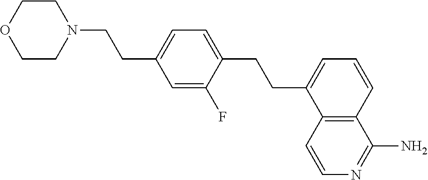 [SMILES: Nc1nccc2c(CCc3ccc(CCN4CCOCC4)cc3F)cccc12]

### 2-Fluoro-4-(2-morpholino-2-oxo-ethyl)benzonitrile

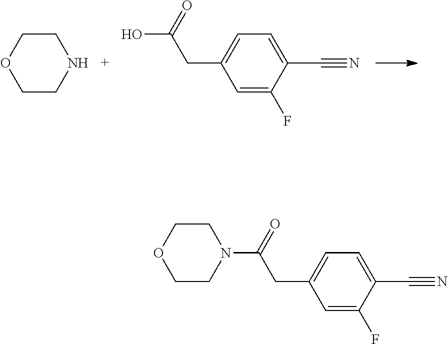

Following General Method 8, 2-(4-cyano-3-fluorophenyl)acetic acid (150 mg, 0.84 mmol) was coupled to morpholine (87 μL, 1.0 mmol). The crude product was purified by flash chromatography (Silica, 0-20% MeOH in DCM) to afford the product (123 mg, 59% yield) as a white solid.

[M+H]⁺=249.0

### [2-Fluoro-4-(2-morpholinoethyl)phenyl]methanamine

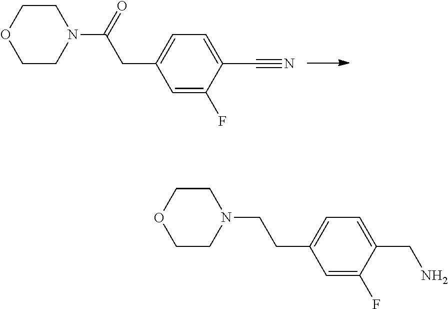 [SMILES: N#Cc1ccc(CC(=O)N2CCOCC2)cc1F ; NCc1ccc(CCN2CCOCC2)cc1F]

A global reduction of the amide and nitrile of 2-fluoro-4-(2-morpholino-2-oxo-ethyl)benzonitrile (120 mg, 0.48 mmol) was performed using General Method 3b, over 2 h. The product was isolated (165 mg, quantitative yield) as a yellow solid and used without further purification.

[M+H]⁺=239.1

### N1-[(2,4-dimethoxyphenyl)methyl]-N5-[[2-fluoro-4-(2-morpholinoethyl)phenyl]methyl]isoquinoline-1,5-diamine

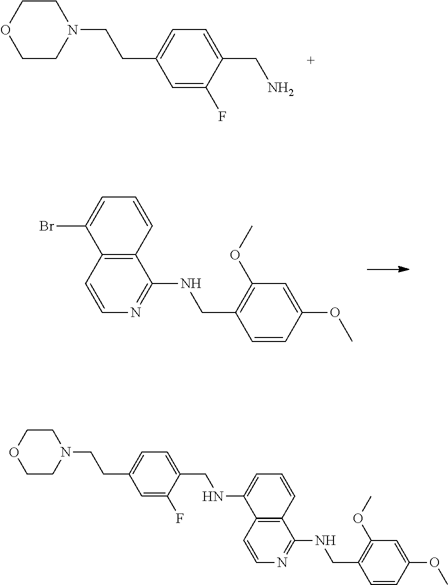 [SMILES: NCc1ccc(CCN2CCOCC2)cc1F ; COc1ccc(CNc2nccc3c(Br)cccc23)c(OC)c1 ; COc1ccc(CNc2nccc3c(NCc4ccc(CCN5CCOCC5)cc4F)cccc23)c(OC)c1]

Following General Method 4, 2-fluoro-4-(2-morpholinoethyl)phenyl]methanamine (50.0 mg, 0.21 mmol) was reacted with 5-bromo-N-[(2,4-dimethoxyphenyl)methyl]isoquinolin-1-amine (117 mg, 0.31 mmol) and NaOtBu (138 mg, 0.42 mmol) in 1,4-dioxane (5 mL) at 60° C. for 2 h. The reaction was quenched with AcOH (43 μL, 0.72 mmol), filtered through Celite®, washing with EtOAc (50 mL) and EtOAc/MeOH (5:1, 60 mL) and concentrated. Purification was performed by flash chromatography (0-65% MeOH in DCM) to afford the product (93 mg, 83% yield) as a brown oil.

[M+H]⁺=531.3

### N5-[[2-fluoro-4-(2-morpholinoethyl)phenyl]methyl]isoquinoline-1,5-diamine

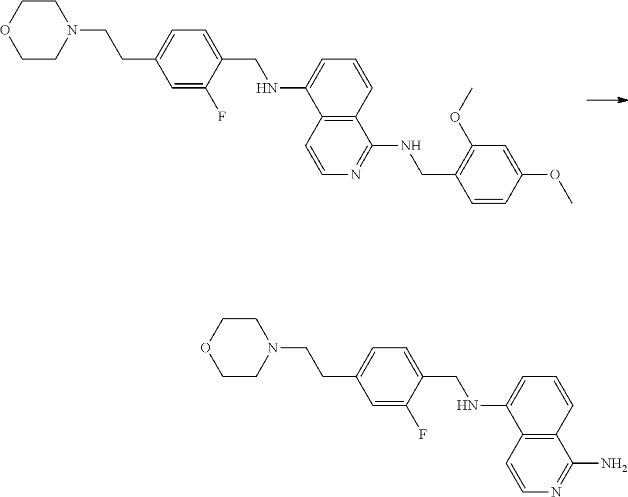 [SMILES: COc1ccc(CNc2nccc3c(NCc4ccc(CCN5CCOCC5)cc4F)cccc23)c(OC)c1 ; Nc1nccc2c(NCc3ccc(CCN4CCOCC4)cc3F)cccc12]

Using General Method 12, N1-[(2,4-dimethoxyphenyl)methyl]-N5-[[2-fluoro-4-(2-morpholinoethyl)phenyl]methyl]isoquinoline-1,5-diamine (93 mg, 0.05 mmol) was deprotected. The crude product was purified via automated prep HPLC (Mass directed 2-60% over 20 min in basic mobile phase). Lyophilisation afforded the product (6 mg, 30% yield) as a white solid.

[M+H]⁺=381.2

¹H NMR (DMSO, 400 MHz) δ 2.36-2.41 (4H, m), 2.44-2.49 (2H, m), 2.70 (2H, dd, J=8.8, 6.6 Hz), 3.55 (4H, t, J=4.6 Hz), 4.44 (2H, d, J=5.8 Hz), 6.45 (1H, d, J=7.6 Hz), 6.50 (2H, s), 6.65 (1H, t, J=6.0 Hz), 6.96 (1H, dd, J=7.8, 1.6 Hz), 7.08 (1H, dd, J=11.5, 1.6 Hz), 7.14 (1H, t, J=8.0 Hz), 7.17-7.26 (2H, m), 7.32 (1H, d, J=8.3 Hz), 7.74 (1H, d, J=6.1 Hz)

### Example Number 2184

### 4-Chloro-N6-[[2-fluoro-4-[2-(4-methylpiperazin-1-yl)ethyl]phenyl]methyl]isoquinoline-1,6-diamine

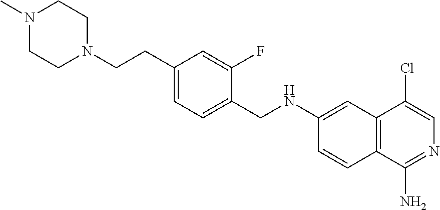 [SMILES: CN1CCN(CCc2ccc(CNc3ccc4c(N)ncc(Cl)c4c3)c(F)c2)CC1]

### 2-Fluoro-4-[2-(4-methylpiperazin-1-yl)-2-oxo-ethyl]benzonitrile

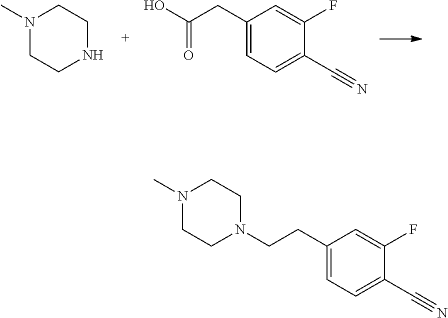

Following General Method 8, (4-cyano-3-fluorophenyl)acetic acid (150 mg, 0.84 mmol) was coupled to 1-methyl piperazine (0.1 mL, 0.92 mmol). The crude product was purified by flash chromatography (Silica, 0-5% (10% NH₃ in MeOH) in DCM) to afford the product (48 mg, 22% yield) as a brown oil.

[M+H]⁺=262.1

### [2-Fluoro-4-[2-(4-methylpiperazin-1-yl)ethyl]phenyl]methanamine

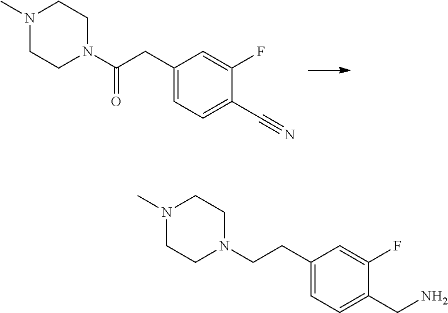 [SMILES: CN1CCN(C(=O)Cc2ccc(C#N)c(F)c2)CC1 ; CN1CCN(CCc2ccc(CN)c(F)c2)CC1]

A global reduction of the amide and nitrile of 2-fluoro-4-[2-(4-methylpiperazin-1-yl)-2-oxo-ethyl]benzonitrile (48.0 mg, 0.18 mmol) was performed using General Method 3b. The product was isolated (46.0 mg, 100% yield) as an off white solid and used without further purification.

[M+H]⁺=252.1

### Methyl N-[4-chloro-6-[[2-fluoro-4-[2-(4-methylpiperazin-1-yl)ethyl]phenyl]methylamino]-1-isoquinolyl]carbamate

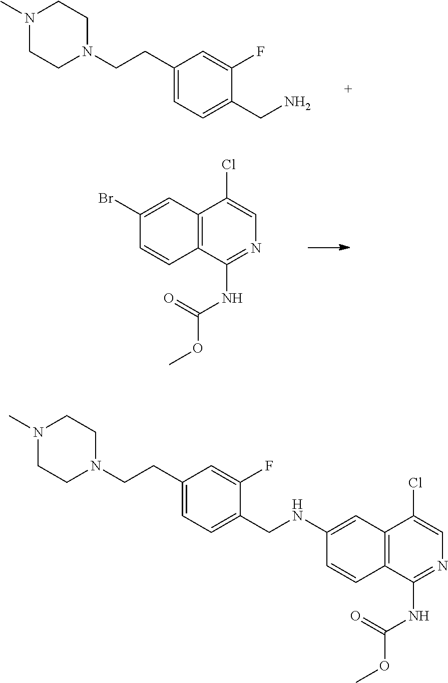 [SMILES: CN1CCN(CCc2ccc(CN)c(F)c2)CC1 ; COC(=O)Nc1ncc(Cl)c2cc(Br)ccc12 ; COC(=O)Nc1ncc(Cl)c2cc(NCc3ccc(CCN4CCN(C)CC4)cc3F)ccc12]

Using General Method 4, methyl N-(6-bromo-4-chloro-1-isoquinolyl)carbamate (21 mg, 0.07 mmol), was reacted with [2-fluoro-4-[2-(4-methylpiperazin-1-yl)ethyl]phenyl]methanamine (20 mg, 0.08 mmol) and NaOtBu (78. mg, 0.24 mmol) in THF (5 mL) at 40° C. for 18 h. After concentrating in vacuo, the residue was purified by flash chromatography (0-20% (10% NH₃ in MeOH) in EtOAc) to afford the product (10 mg, 26% yield) as a yellow solid.

[M+H]⁺=486.1

### 4-Chloro-N6-[[2-fluoro-4-[2-(4-methylpiperazin-1-yl)ethyl]phenyl]methyl]isoquinoline-1,6-diamine

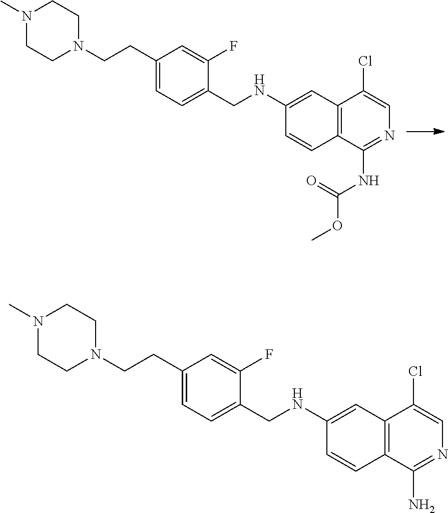 [SMILES: COC(=O)Nc1ncc(Cl)c2cc(NCc3ccc(CCN4CCN(C)CC4)cc3F)ccc12 ; CN1CCN(CCc2ccc(CNc3ccc4c(N)ncc(Cl)c4c3)c(F)c2)CC1]

Following General Method 14a, methyl N-[4-chloro-6-[[2-fluoro-4-[2-(4-methylpiperazin-1-yl)ethyl]phenyl]methylamino]-1-isoquinolyl]carbamate (10 mg, 0.02 mmol) was deprotected over 24 h. The reaction mixture was concentrated and purified via automated prep HPLC (Mass directed 2-60% over 20 min in basic mobile phase). Lyophilisation afford the product (1 mg, 12% yield) as a white solid.

[M+H]⁺=428.1

¹H NMR (CDCl₃, 400 MHz) δ 2.30 (3H, s), 2.35-2.85 (12H, m), 4.50 (2H, d, J=5.7 Hz), 4.56-4.64 (1H, m), 4.93 (2H, s), 6.87 (1H, dd, J=9.0, 2.4 Hz), 6.92-7.00 (2H, m), 7.04 (1H, d, J=2.3 Hz), 7.30 (1H, t, J=7.7 Hz), 7.57 (1H, d, J=9.0 Hz), 7.85 (1H, s)

### Example Number 2212

### N5-[[2-fluoro-4-[2-[(1S,4S)-5-isopropyl-2,5-diazabicyclo[2.2.1]heptan-2-yl]ethyl]phenyl]methyl]isoquinoline-1,5-diamine

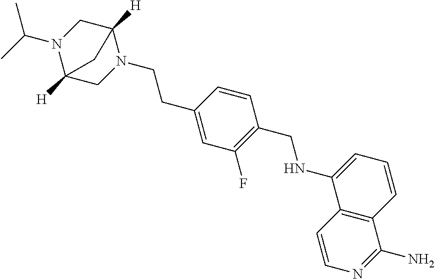 [SMILES: CC(C)N1C[C@H]2C[C@@H]1CN2CCc1ccc(CNc2cccc3c(N)nccc23)c(F)c1]

### 2-Fluoro-4-[2-[(1S,4S)-5-isopropyl-2,5-diazabicyclo[2.2.1]heptan-2-yl]-2-oxo-ethyl]benzonitrile

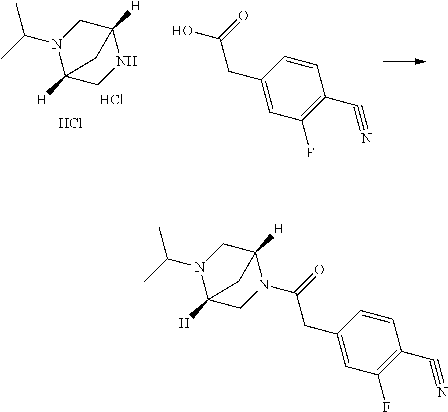

Following General Method 8, (1S,4S)-2-isopropyl-2,5-diazabicyclo[2.2.1]heptane;dihydrochloride (200 mg, 0.93 mmol) was coupled with 2-(4-cyano-3-fluorophenyl)acetic acid (185 mg, 1.03 mmol). The crude product was purified by flash chromatography (Silica, 0-20% (10% NH₃ in MeOH) in DCM) to afford the product (225 mg, 80% yield) as a pale brown gum.

[M+H]⁺=302.1

¹H NMR (400 MHz, CDCl₃) δ 1.04-1.07 (6H, m), 1.67-1.99 (2H, m), 2.33-2.65 (2H, m), 3.04-3.43 (2H, m), 3.57-3.80 (4H, m), 4.29 and 4.72 (1H, s), 7.18-7.23 (2H, m), 7.55-7.61 (1H, m)

### [2-Fluoro-4-[2-[(1S,4S)-5-isopropyl-2,5-diazabicyclo[2.2.1]heptan-2-yl]ethyl]phenyl]methanamine

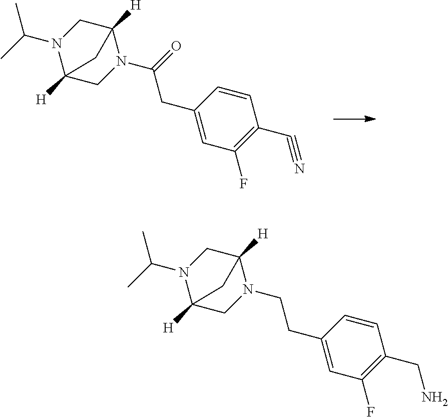 [SMILES: CC(C)N1C[C@H]2C[C@@H]1CN2C(=O)Cc1ccc(C#N)c(F)c1 ; CC(C)N1C[C@H]2C[C@@H]1CN2CCc1ccc(CN)c(F)c1]

A global reduction of the nitrile and amide of 2-fluoro-4-[2-[(1S,4S)-5-isopropyl-2,5-diazabicyclo[2.2.1]heptan-2-yl]-2-oxo-ethyl]benzonitrile (225 mg, 0.75 mmol) was performed using General Method 3b at rt and stirred for 13 h. The product was isolated (175 mg, 0.60 mmol, 80% yield) as a yellow oil and used without further purification.

[M+H]⁺=292.1

### N1-[(2,4-dimethoxyphenyl)methyl]-N5-[[2-fluoro-4-[2-[(1S,4S)-5-isopropyl-2,5-diazabicyclo[2.2.1]heptan-2-yl]ethyl]phenyl]methyl]isoquinoline-1,5-diamine

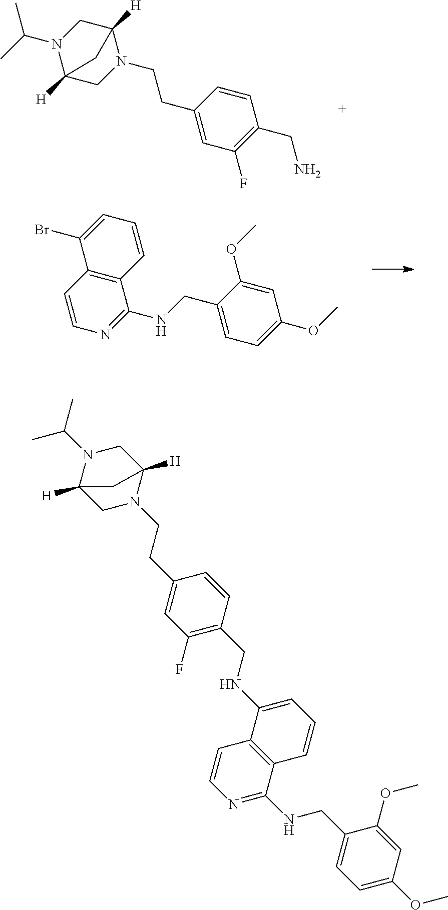

Following General Method 4, [2-fluoro-4-[2-[(1S,4S)-5-isopropyl-2,5-diazabicyclo[2.2.1]heptan-2-yl]ethyl]phenyl]methanamine (71 mg, 0.24 mmol) was reacted with 5-bromo-N-[(2,4-dimethoxyphenyl)methyl]isoquinolin-1-amine (91 mg, 0.24 mmol) and Cs₂CO₃ (176 mg, 0.54 mmol) in 1,4-dioxane (3 mL) at 60° C. for 5 days. The reaction mixture was cooled, filtered over Celite®, washed with EtOAc (80 mL) and MeOH (3 mL) to afford the crude product (108 mg, 76% yield) as a brown oil, which was used without purification.

[M+H]⁺=584.1

### N5-[[2-fluoro-4-[2-[(1S,4S)-5-isopropyl-2,5-diazabicyclo[2.2.1]heptan-2-yl]ethyl]phenyl]methyl]isoquinoline-1,5-diamine

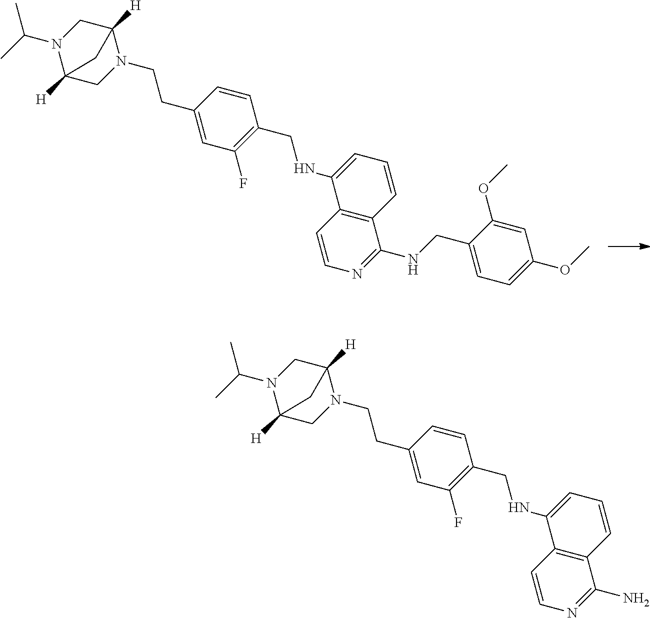

Using General Method 12, N1-[(2,4-dimethoxyphenyl)methyl]-N5-[[2-fluoro-4-[2-[(1S,4S)-5-isopropyl-2,5-diazabicyclo[2.2.1]heptan-2-yl]ethyl]phenyl]methyl]isoquinoline-1,5-diamine (108 mg, 0.19 mmol) was deprotected. The crude product was purified via automated prep HPLC. (Mass directed 2-60% over 20 min in basic mobile phase). Lyophilisation afforded the product (5 mg, 6% yield) as a white solid.

[M+H]⁺=434.2

¹H NMR (DMSO-d6, 400 MHz) δ 0.93 (6H, dd, J=14.9, 6.1 Hz), 1.50 (2H, q, J=9.0 Hz), 2.40 (1H, d, J=9.4 Hz), 2.52-2.65 (6H, m), 2.65-2.74 (2H, m), 3.20 (2H, s), 4.44 (2H, d, J=5.8 Hz), 6.45 (1H, d, J=7.7 Hz), 6.49 (2H, s), 6.65 (1H, t, J=6.0 Hz), 6.95 (1H, dd, J=7.8, 1.6 Hz), 7.07 (1H, dd, J=11.5, 1.6 Hz), 7.13 (1H, t, J=8.0 Hz), 7.17-7.24 (2H, m), 7.32 (1H, d, J=8.3 Hz), 7.74 (1H, d, J=6.1 Hz)

### Example Number 2213

### N5-[[2-fluoro-4-[2-[(1R,4R)-5-isopropyl-2,5-diazabicyclo[2.2.1]heptan-2-yl]ethyl]phenyl]methyl]isoquinoline-1,5-diamine

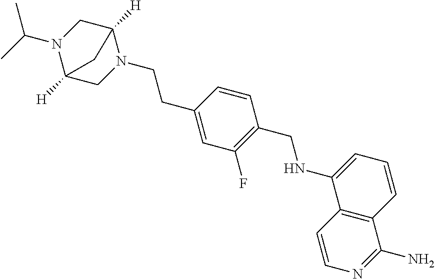 [SMILES: CC(C)N1C[C@@H]2C[C@H]1CN2CCc1ccc(CNc2cccc3c(N)nccc23)c(F)c1]

### Tert-butyl-(1R,4R)-5-isopropyl-2,5-diazabicyclo[2.2.1]heptane-2-carboxylate

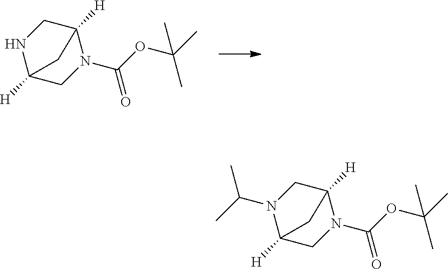 [SMILES: CC(C)(C)OC(=O)N1C[C@@H]2C[C@H]1CN2 ; CC(C)N1C[C@@H]2C[C@H]1CN2C(=O)OC(C)(C)C]

To a solution of (1R,4R)-tert-Butyl 2,5-diazabicyclo[2.2.1]heptane-2-carboxylate (500 mg, 2.52 mmol) in THF (10 mL) was added acetone (1.0 mL, 13.62 mmol) and the reaction was stirred for 15 min, before adding sodium triacetoxyborohydride (1.6 g, 7.57 mmol). The reaction mixture was stirred at rt for 18 h, before diluting with DCM (50 mL) and NaHCO₃ (sat. aq. 15 mL). The aqueous layer was re-extracted with DCM (2×20 mL). The combined organics were washed with additional NaHCO₃ (sat. aq. 15 mL), dried (MgSO₄), filtered and concentrated to afford the product (604 mg, 100% yield) as a colourless oil.

[M+H]⁺=241.1

¹H NMR (CDCl₃, 400 MHz) δ 0.98-1.13 (6H, m), 1.45 (9H, s), 1.65-1.75 (1H, m), 1.81-1.87 (1H, m), 2.45 (1H, dd, J=52.7, 9.6 Hz), 2.55-2.70 (1H, m), 3.01-3.17 (2H, m), 3.52 (1H, dd, J=34.8, 10.3 Hz), 3.68 (1H, s), 4.26 (1H, d, J=47.9 Hz) ppm.

### 2-Isopropyl-2,5-diazabicyclo[2.2.1]heptane;dihydrochloride

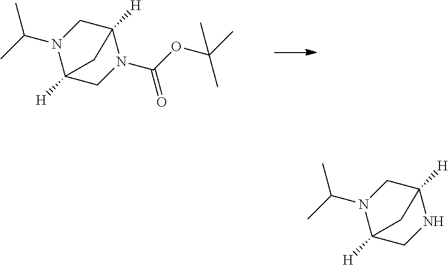

Boc deprotection of (1R,4R)-tert-butyl 5-isopropyl-2,5-diazabicyclo[2.2.1]heptane-2-carboxylate (604 mg, 2.51 mmol) was performed using General Method 7a. The reaction mixture was concentrated to obtain the product (601 mg, Quantitative yield) as a white solid.

[M+H]⁺=141.0

### 2-Fluoro-4-[2-[(1R,4R)-5-isopropyl-2,5-diazabicyclo[2.2.1]heptan-2-yl]-2-oxo-ethyl]benzonitrile

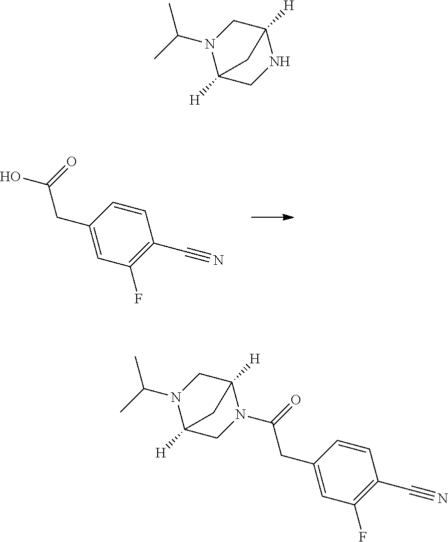

Using General Method 8, (1R,4R)-2-isopropyl-2,5-diazabicyclo[2.2.1]heptane;dihydrochloride (325 mg, 1.52 mmol) was coupled with 2-(4-cyano-3-fluorophenyl)acetic acid (301 mg, 1.68 mmol. The crude product was purified by flash chromatography (Silica, 0-10% (10% NH₃ in MeOH) in DCM) to afford (267 mg, 58% yield) as a colourless oil.

[M+H]⁺=302.1

### [2-Fluoro-4-[2-[(1R,4R)-5-isopropyl-2,5-diazabicyclo[2.2.1]heptan-2-yl]ethyl]phenyl]methanamine

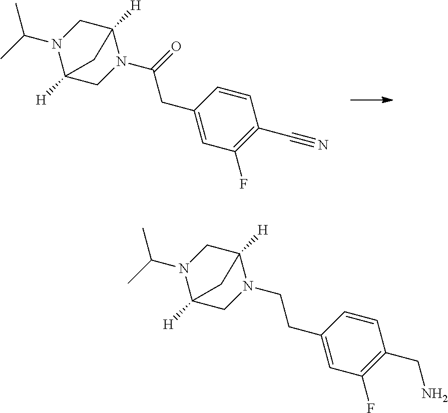 [SMILES: CC(C)N1C[C@@H]2C[C@H]1CN2C(=O)Cc1ccc(C#N)c(F)c1 ; CC(C)N1C[C@@H]2C[C@H]1CN2CCc1ccc(CN)c(F)c1]

A global reduction of the amide and nitrile, 2-fluoro-4-[2-[(1R,4R)-5-isopropyl-2,5-diazabicyclo[2.2.1]heptan-2-yl]-2-oxo-ethyl]benzonitrile (218 mg, 0.72 mmol) was performed using General Method 3b for 13 h. The product was isolated as a yellow oil (186 mg, 88% yield) and used without further purification.

[M+H]⁺=292.1

### N1-[(2,4-dimethoxyphenyl)methyl]-N5-[[2-fluoro-4-[2-[(1R,4R)-5-isopropyl-2,5-diazabicyclo[2.2.1]heptan-2-yl]ethyl]phenyl]methyl]isoquinoline-1,5-diamine

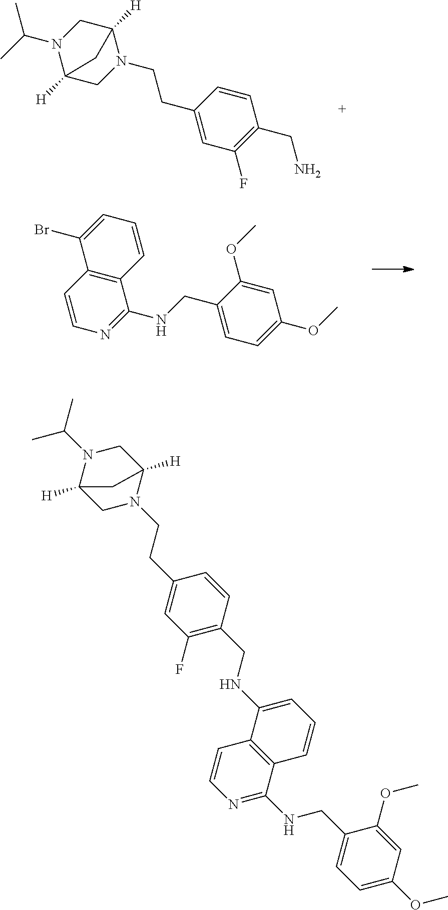

Following General Method 4, [2-fluoro-4-[2-[(1R,4R)-5-isopropyl-2,5-diazabicyclo[2.2.1]heptan-2-yl]ethyl]phenyl]methanamine (71 mg, 0.24 mmol) was reacted with 5-bromo-N-[(2,4-dimethoxyphenyl)methyl]isoquinolin-1-amine (91 mg, 0.24 mmol) and Cs₂CO₃ (176 mg, 0.54 mmol) in 1,4-dioxane (3 mL) at 60° C. for 18 h. After quenching and filtering through Celite®, the product (271 mg, 100% yield) was obtained as a brown oil and used directly.

[M+H]⁺=584.3

### N5-[[2-fluoro-4-[2-[(1R,4R)-5-isopropyl-2,5-diazabicyclo[2.2.1]heptan-2-yl]ethyl]phenyl]methyl]isoquinoline-1,5-diamine

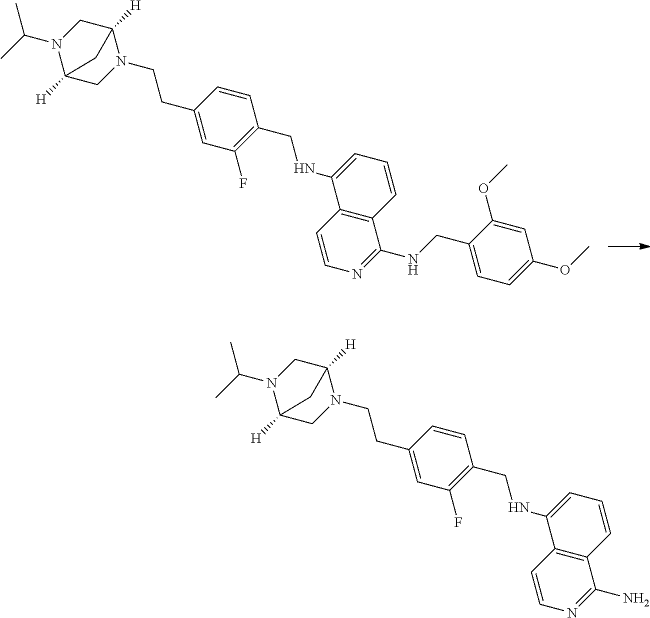

Following General Method 12, N1-[(2,4-dimethoxyphenyl)methyl]-N5-[[2-fluoro-4-[2-[(1R,4R)-5-isopropyl-2,5-diazabicyclo[2.2.1]heptan-2-yl]ethyl]phenyl]methyl]isoquinoline-1,5-diamine (142 mg, 0.24 mmol) was deprotected. The crude material was purified via automated prep HPLC (Mass directed 2-60% over 20 min in acidic mobile phase) to afford the product (22 mg, 17% yield) as a brown solid.

[M+H]⁺=434.2

¹H NMR (DMSO, 400 MHz) δ 1.06 (6H, dd, J=21.2, 6.2 Hz), 1.72 (2H, q, J=10.2 Hz), 2.62-2.98 (9H, m), 3.44 (1H, s), 3.80 (1H, s), 4.45 (2H, d, J=5.7 Hz), 6.45 (1H, d, J=7.7 Hz), 6.61 (2H, s), 6.68 (1H, t, J=6.0 Hz), 6.97 (1H, dd, J=7.9, 1.6 Hz), 7.06-7.27 (4H, m), 7.33 (1H, d, J=8.4 Hz), 7.74 (1H, d, J=6.1 Hz), 8.25 (2H, s)

### Example Number 1041

### 4-Chloro-N6-[[6-(5,6,7,8-tetrahydroimidazo[1,2-a]pyridin-7-ylmethoxy)-3-pyridyl]methyl]isoquinoline-1,6-diamine

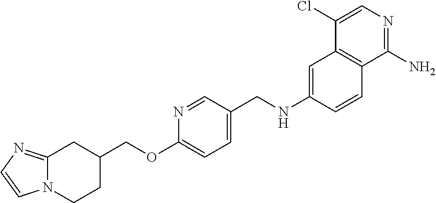 [SMILES: Nc1ncc(Cl)c2cc(NCc3ccc(OCC4CCn5ccnc5C4)nc3)ccc12]

### 6-((5,6,7,8-Tetrahydroimidazo[1,2-a]pyridin-6-yl)methoxy)nicotinonitrile

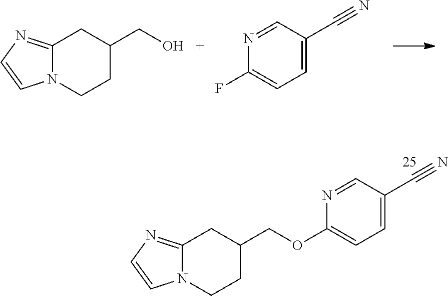 [SMILES: OCC1CCn2ccnc2C1 ; N#Cc1ccc(F)nc1 ; N#Cc1ccc(OCC2CCn3ccnc3C2)nc1]

Following General Method 1b, (5,6,7,8-tetrahydroimidazo[1,2-a]pyridin-6-yl)methanol (650 mg, 4.27 mmol) was reacted with 6-fluoronicotinonitrile (626 mg, 5.12 mmol) for 18 h. The solids were removed by filtration and the filtrate concentrated. The crude product was purified by flash chromatography (Silica, 1-5% (0.7M NH₃ in MeOH) in DCM) to afford the product (676 mg, 59% yield) as an orange solid.

[M+H]⁺=255.1

¹H NMR (500 MHz, DMSO-d6) 1.62-1.77 (1H, m), 2.02-2.12 (1H, m), 2.45-2.50 (1H, m), 2.66-2.76 (1H, m), 2.79-2.90 (1H, m), 3.69-3.80 (1H, m), 4.18 (1H, dd, J=12.4, 5.2 Hz), 4.35 (1H, dd, J=10.7, 7.3 Hz), 4.45 (1H, dd, J=10.7, 6.0 Hz), 6.79-6.83 (1H, m), 6.97-7.02 (1H, m), 7.03-7.08 (1H, m), 8.15-8.21 (1H, m), 8.68-8.73 (1H, m)

### [6-(5,6,7,8-Tetrahydroimidazo[1,2-a]pyridin-7-ylmethoxy)-3-pyridyl]methanamine

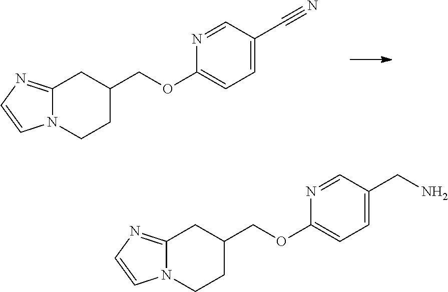 [SMILES: N#Cc1ccc(OCC2CCn3ccnc3C2)nc1 ; NCc1ccc(OCC2CCn3ccnc3C2)nc1]

Reduction of the nitrile, 6-(5,6,7,8-tetrahydroimidazo[1,2-a]pyridin-7-ylmethoxy)pyridine-3-carbonitrile (140 mg, 0.55 mmol) was performed following General Method 3a, using Raney Ni over 30 min. The solvent was removed in vacuo to afford the product (138 mg, 97% yield) as a colourless oil.

[M+H]⁺=259.1

¹H NMR (CDCl₃, 400 MHz) δ 1.73-1.85 (1H, m), 2.10-2.22 (1H, m), 2.50-2.63 (1H, m), 2.78-2.92 (1H, m), 2.98-3.11 (1H, m), 3.71-3.79 (1H, m), 3.81 (2H, s), 4.20 (1H, dd, J=12.2, 5.2 Hz), 4.24-4.30 (1H, m), 4.39-4.45 (1H, m), 6.73 (1H, d, J=8.4 Hz), 6.76-6.81 (1H, m), 6.98 (1H, d, J=1.3 Hz), 7.59 (1H, dd, J=8.5, 2.6 Hz), 8.05 (1H, d, J=2.5 Hz)

### Methyl N-[4-chloro-6-[[6-(5,6,7,8-tetrahydroimidazo[1,2-a]pyridin-7-ylmethoxy)-3-pyridyl]methylamino]-1-isoquinolyl]carbamate

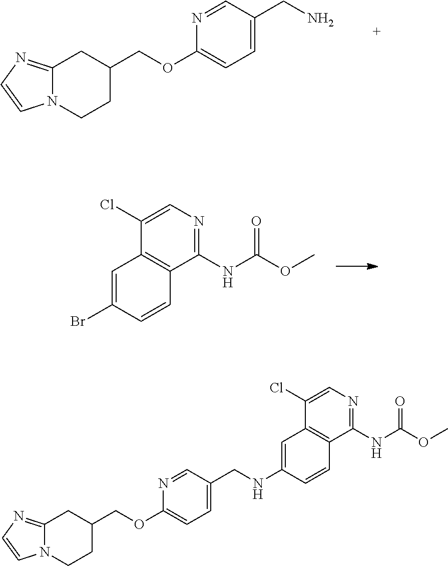 [SMILES: NCc1ccc(OCC2CCn3ccnc3C2)nc1 ; COC(=O)Nc1ncc(Cl)c2cc(Br)ccc12 ; COC(=O)Nc1ncc(Cl)c2cc(NCc3ccc(OCC4CCn5ccnc5C4)nc3)ccc12]

Following General Method 4, [6-(5,6,7,8-tetrahydroimidazo[1,2-a]pyridin-7-ylmethoxy)-3-pyridyl]methanamine (135 mg, 0.47 mmol) was reacted with methyl N-(6-bromo-4-chloro-1-isoquinolyl)carbamate (147 mg, 0.47 mmol) and NaOtBu (305 mg, 0.93 mmol) in THF (5 mL) at 40° C. for 1 h. The mixture was cooled to rt, quenched with AcOH (53 μL, 0.93 mmol) and concentrated. The residue was purified by flash chromatography (Silica, 0-80% (2% NH₄ in EtOAc:EtOH (3:1)) in Pet ether 60-80) to afford the product (219 mg, 96% yield) as a pale yellow oil.

[M+H]⁺=493.1

### 4-Chloro-N6-[[6-(5,6,7,8-tetrahydroimidazo[1,2-a]pyridin-7-ylmethoxy)-3-pyridyl]methyl]isoquinoline-1,6-diamine

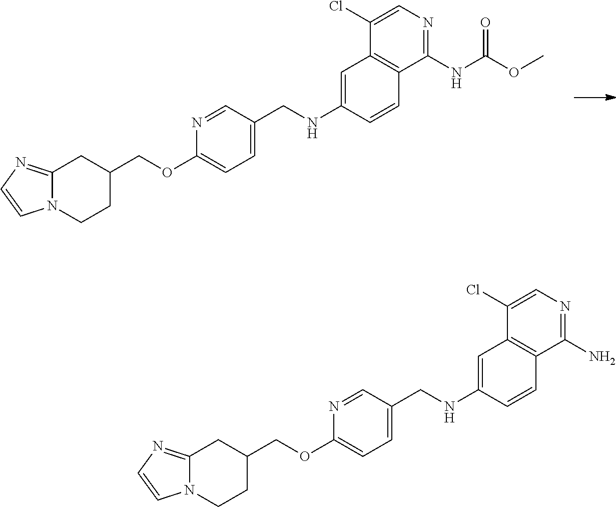 [SMILES: COC(=O)Nc1ncc(Cl)c2cc(NCc3ccc(OCC4CCn5ccnc5C4)nc3)ccc12 ; Nc1ncc(Cl)c2cc(NCc3ccc(OCC4CCn5ccnc5C4)nc3)ccc12]

Deprotection of methyl N-[4-chloro-6-[[6-(5,6,7,8-tetrahydroimidazo[1,2-a]pyridin-7-ylmethoxy)-3-pyridyl]methylamino]-1-isoquinolyl]carbamate (219 mg, 0.44 mmol) was carried out according to General Method 14a over 72 h. The reaction was cooled to rt, quenched with AcOH (0.1 mL, 2.0 mmol) and purified by SCX, eluting in 7M NH₃ in MeOH. The product was isolated as a white solid following lyophilisation (98 mg, 51% yield).

[M+H]⁺=435.1

¹H NMR (DMSO-d6, 400 MHz) δ 1.59-1.73 (1H, m), 1.96-2.12 (1H, m), 2.45 (1H, br s), 2.64-2.77 (1H, m), 2.77-2.88 (1H, m), 3.72 (1H, dd, J=12.3, 10.1 Hz), 4.16 (1H, dd, J=12.3, 5.2 Hz), 4.22 (1H, dd, J=10.7, 7.4 Hz), 4.32 (1H, d, J=6.0 Hz), 4.34 (2H, d, J=5.6 Hz), 6.55 (2H, s), 6.71 (1H, d, J=2.3 Hz), 6.79 (1H, d, J=1.2 Hz), 6.82-6.86 (1H, m), 6.95 (1H, dd, J=9.1, 2.4 Hz), 6.97-7.02 (1H, m), 7.06 (1H, t, J=5.8 Hz), 7.65 (1H, s), 7.74 (1H, dd, J=8.5, 2.5 Hz), 7.92 (1H, d, J=9.0 Hz), 8.20 (1H, d, J=2.4 Hz)

### Example Number 4298

### N5-((2-((5,6,7,8-tetrahydroimidazo[1,2-a]pyridin-7-yl)methoxy)pyridin-4-yl)methyl)isoquinoline-1,5-diamine

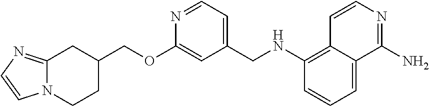 [SMILES: Nc1nccc2c(NCc3ccnc(OCC4CCn5ccnc5C4)c3)cccc12]

### N1-(2,4-dimethoxybenzyl)-N5-((2-((5,6,7,8-tetrahydroimidazo[1,2-a]pyridin-7-yl)methoxy)pyridin-4-yl)methyl)isoquinoline-1,5-diamine

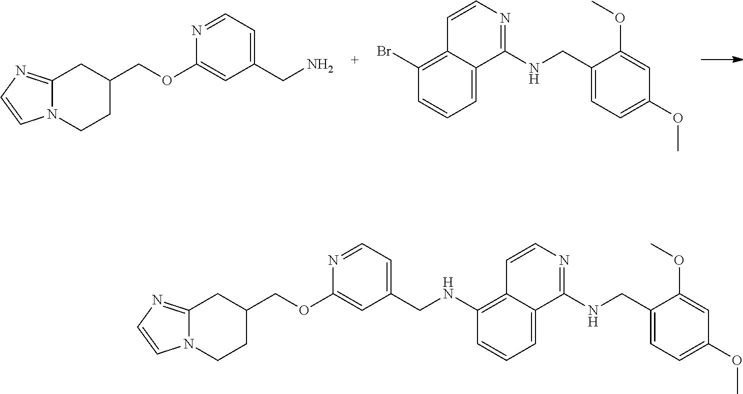 [SMILES: NCc1ccnc(OCC2CCn3ccnc3C2)c1 ; COc1ccc(CNc2nccc3c(Br)cccc23)c(OC)c1 ; COc1ccc(CNc2nccc3c(NCc4ccnc(OCC5CCn6ccnc6C5)c4)cccc23)c(OC)c1]

Following General Method 4, (2-((5,6,7,8-tetrahydroimidazo[1,2-a]pyridin-7-yl)methoxy)pyridin-4-yl)methanamine (108 mg, 0.42 mmol) was reacted with 5-bromo-N-(2,4-dimethoxybenzyl)isoquinolin-1-amine (156 mg, 0.42 mmol) and NaOtBu (80 mg, 0.84 mmol) in 1,4-dioxane (5 mL) at 60° C. for 1 h. The reaction mixture was cooled to rt and concentrated before purification by flash chromatography (Silica, 0-80% (2% NH₃ in EtOAc/EtOH (3:1)) in Pet ether) to afford the product (85 mg, 0.13 mmol, 32% yield) as a yellow oil.

[M+H]⁺=551.2

### N5-((2-((5,6,7,8-tetrahydroimidazo[1,2-a]pyridin-7-yl)methoxy)pyridin-4-yl)methyl)isoquinoline-1,5-diamine

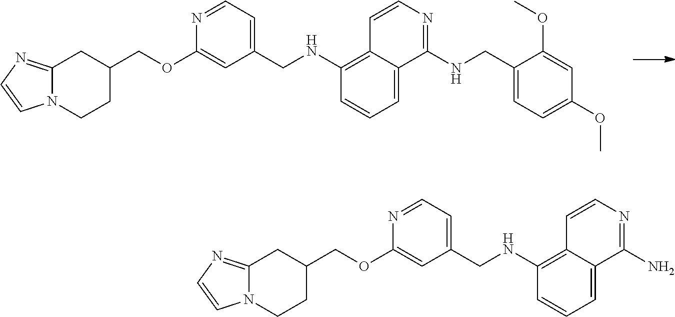 [SMILES: COc1ccc(CNc2nccc3c(NCc4ccnc(OCC5CCn6ccnc6C5)c4)cccc23)c(OC)c1 ; Nc1nccc2c(NCc3ccnc(OCC4CCn5ccnc5C4)c3)cccc12]

Deprotection of N1-(2,4-dimethoxybenzyl)-N5-((2-((5,6,7,8-tetrahydroimidazo[1,2-a]pyridin-7-yl)methoxy)pyridin-4-yl)methyl)isoquinoline-1,5-diamine (200 mg, 0.36 mmol) was performed using General Method 12. Purification was performed by flash chromatography (Silica, 0-100% (2% NH₃ in EtOAc/MeCN/EtOH (3:3:1)) in Pet.Ether), followed by automated prep HPLC. (Mass directed 2-60% over 20 min in basic mobile phase). Lyophilisation afforded the product (30 mg, 21% yield) as a white solid.

[M+H]⁺=401.2

¹H NMR (DMSO-d6, 400 MHz) δ 1.57-1.77 (1H, m), 2.05-2.15 (1H, m), 2.26-2.40 (1H, m), 2.41-2.47 (1H, m), 2.89 (1H, ddd, J=16.2, 5.0, 1.4 Hz), 3.85 (1H, td, J=11.9, 4.7 Hz), 4.00-4.09 (1H, m), 4.22 (2H, dd, J=6.6, 1.6 Hz), 4.45 (2H, d, J=5.9 Hz), 6.38 (1H, dd, J=7.8, 0.9 Hz), 6.51 (2H, s), 6.76-6.78 (1H, m), 6.78 (1H, d, J=1.2 Hz), 6.81 (1H, t, J=6.1 Hz), 6.97 (1H, d, J=1.2 Hz), 6.98 (1H, dd, J=5.3, 1.4 Hz), 7.12 (1H, t, J=8.0 Hz), 7.20 (1H, dd, J=6.3, 0.9 Hz), 7.33 (1H, d, J=8.3 Hz), 7.76 (1H, d, J=6.1 Hz), 8.06 (1H, dd, J=5.3, 0.7 Hz) ppm.

### Example Number 4299

### N5-[[2-[(1-isopropyl-4-piperidyl)methoxy]-4-pyridyl]methyl]isoquinoline-1,5-diamine

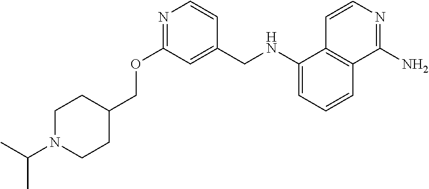 [SMILES: CC(C)N1CCC(COc2cc(CNc3cccc4c(N)nccc34)ccn2)CC1]

### Tert-butyl 4-[(4-cyano-2-pyridyl)oxymethyl]piperidine-1-carboxylate

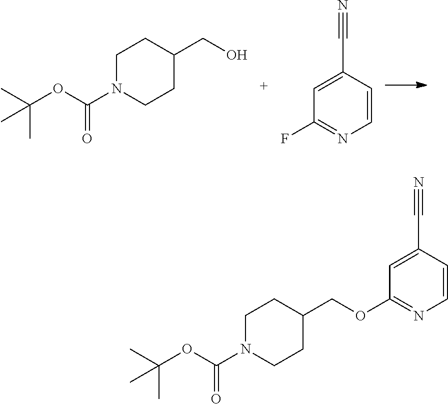 [SMILES: CC(C)(C)OC(=O)N1CCC(CO)CC1 ; N#Cc1ccnc(F)c1 ; CC(C)(C)OC(=O)N1CCC(COc2cc(C#N)ccn2)CC1]

Following General Method 1b, N-boc-4-(hydroxymethyl)piperidine (3523 mg, 1.64 mmol) was reacted with 4-cyano-2-fluoropyridine (200 mg, 1.64 mmol) in MeCN (4 mL) 50° C. for 18 h. The reaction mixture was cooled to rt and diluted with water (10 mL). The product was extracted into DCM (2×25 mL), dried (MgSO₄), filtered and concentrated. The residue was purified by flash chromatography (Silica, 5-100% EtOAc in Pet ether 60-80) to afford the product (500 mg, 96% yield) as a pale yellow oil.

[M-boc+H]⁺=218.1

¹H NMR (400 MHz, CDCl₃) δ 1.21-1.32 (2H, m), 1.47 (9H, s), 1.80 (2H, d, J=12.9 Hz), 1.92-2.02 (1H, m), 2.75 (2H, t, J=11.8 Hz), 4.09-4.20 (4H, m), 6.99 (1H, d, J=0.9 Hz), 7.07 (1H, dd, J=5.1, 1.3 Hz), 8.28 (1H, d, J=5.0 Hz)

### Tert-butyl 4-[[4-(aminomethyl)-2-pyridyl]oxymethyl]piperidine-1-carboxylate

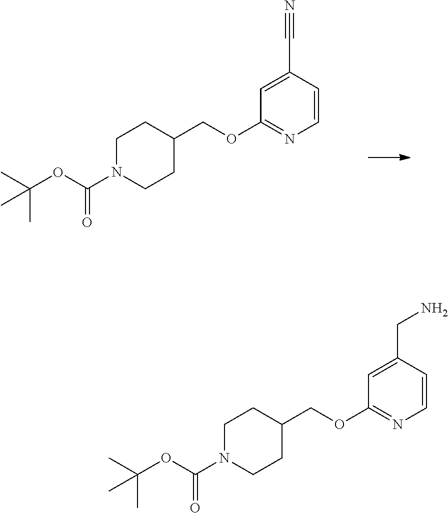 [SMILES: CC(C)(C)OC(=O)N1CCC(COc2cc(C#N)ccn2)CC1 ; CC(C)(C)OC(=O)N1CCC(COc2cc(CN)ccn2)CC1]

The nitrile, tert-butyl 4-[(4-cyano-2-pyridyl)oxymethyl]piperidine-1-carboxylate (500 mg, 1.58 mmol) was reduced according to General Method 3a, using Raney Ni for 1 h. The solvent was removed in vacuo to afford the product (497 mg, 98% yield) as a colourless oil.

[M+H]⁺=322.1

¹H NMR (CDCl₃, 400 MHz) δ 1.25 (2H, qd, J=12.4, 4.4 Hz), 1.46 (9H, s), 1.73-1.83 (2H, m), 1.89-2.00 (1H, m), 2.33 (2H, br s), 2.73 (2H, t, J=12.8 Hz), 3.86 (2H, s), 4.04-4.19 (4H, m), 6.65-6.75 (1H, m), 6.77-6.88 (1H, m), 8.07 (1H, dd, J=5.3, 0.7 Hz)

### Tert-butyl 4-[[4-[[[1-[(2,4-dimethoxyphenyl)methylamino]-5-isoquinolyl]amino]methyl]-2-pyridyl]oxymethyl]piperidine-1-carboxylate

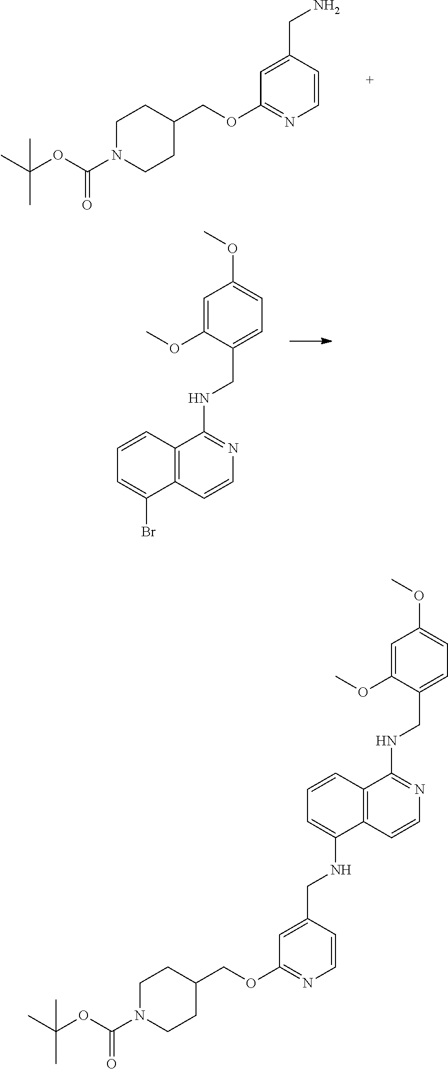 [SMILES: CC(C)(C)OC(=O)N1CCC(COc2cc(CN)ccn2)CC1 ; COc1ccc(CNc2nccc3c(Br)cccc23)c(OC)c1 ; COc1ccc(CNc2nccc3c(NCc4ccnc(OCC5CCN(C(=O)OC(C)(C)C)CC5)c4)cccc23)c(OC)c1]

Using General Method 4, tert-butyl 4-[[4-(aminomethyl)-2-pyridyl]oxymethyl]piperidine-1-carboxylate (497 mg, 1.55 mmol) was reacted with 5-bromo-N-[(2,4-dimethoxyphenyl)methyl]isoquinolin-1-amine (635 mg, 1.7 mmol) and Cs₂CO₃ (1014 mg, 3.09 mmol) in 1,4-dioxane (6 mL) at 60° C. for 18 h. After quenching and filtering through Celite®, the crude product was purified by flash chromatography (Silica, 10-100% EtOAc in Pet ether 60-80) to afford the product (800 mg, 84% yield) as a pale yellow gum.

[M+H]⁺=614.3

¹H NMR (400 MHz, CDCl₃) δ 0.83-0.97 (2H, m), 1.45 (9H, s), 1.59 (3H, s), 1.77-1.99 (3H, m), 2.72 (2H, t, J=12.3 Hz), 3.80 (3H, s), 3.86 (3H, s), 4.47 (2H, d, J=5.5 Hz), 4.72-4.78 (3H, m), 5.63 (1H, t, J=5.3 Hz), 6.44-6.55 (31H, m), 6.75 (1H, s), 6.85-6.90 (21H, m), 7.08 (1H, d, J=8.4 Hz), 7.20-7.32 (31H, m), 8.05 (1H, d, J=6.1 Hz), 8.09 (1H, d, J=5.4 Hz)

### N1-[(2,4-dimethoxyphenyl)methyl]-N5-[[2-(4-piperidylmethoxy)-4-pyridyl]methyl]isoquinoline-1,5-diamine

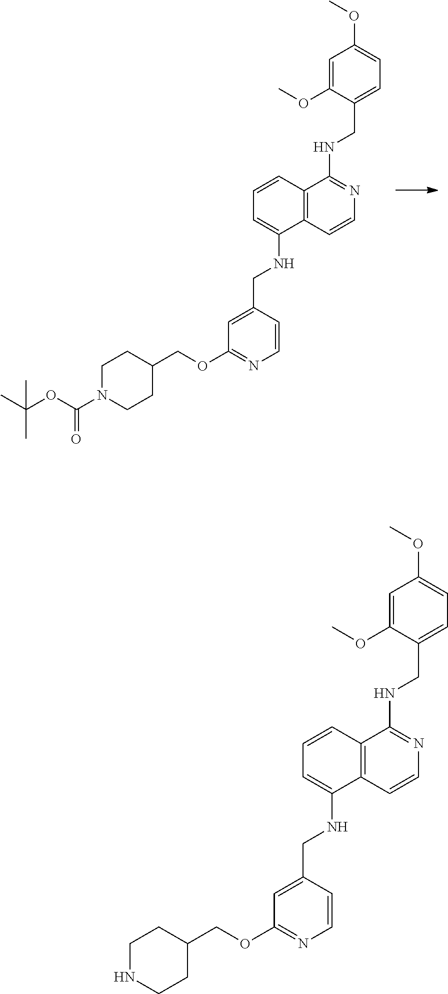 [SMILES: COc1ccc(CNc2nccc3c(NCc4ccnc(OCC5CCN(C(=O)OC(C)(C)C)CC5)c4)cccc23)c(OC)c1 ; COc1ccc(CNc2nccc3c(NCc4ccnc(OCC5CCNCC5)c4)cccc23)c(OC)c1]

Boc deprotection of tert-butyl 4-[[4-[[[1-[(2,4-dimethoxyphenyl)methylamino]-5-isoquinolyl]amino]methyl]-2-pyridyl]oxymethyl]piperidine-1-carboxylate (800 mg, 1.3 mmol) was carried out using General Method 7b. The reaction mixture was concentrated, converted to free base using a bicarbonate cartridge and triturated with Et₂O (20 mL) to afford the product (708 mg, 97% yield) as an orange oil.

[M+H]⁺=514.2

### N1-[(2,4-dimethoxyphenyl)methyl]-N5-[[2-[(1-isopropyl-4-piperidyl)methoxy]-4-pyridyl]methyl]isoquinoline-1,5-diamine

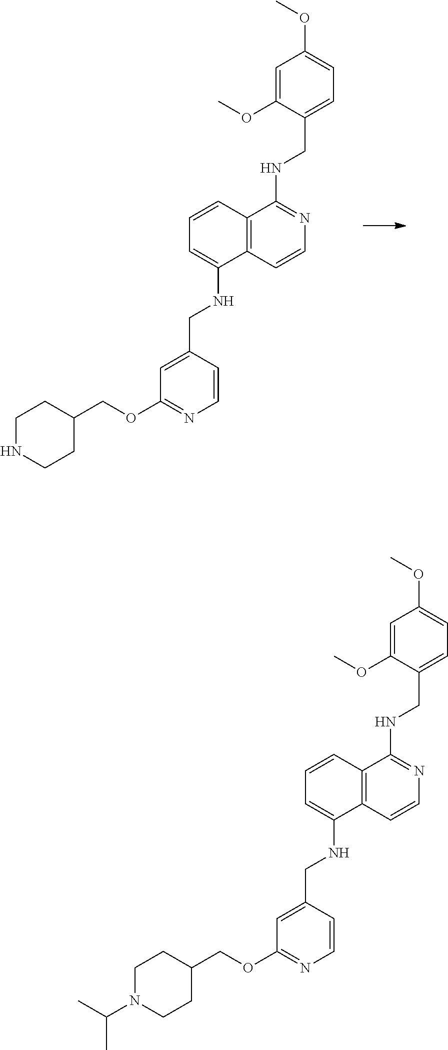 [SMILES: COc1ccc(CNc2nccc3c(NCc4ccnc(OCC5CCNCC5)c4)cccc23)c(OC)c1 ; COc1ccc(CNc2nccc3c(NCc4ccnc(OCC5CCN(C(C)C)CC5)c4)cccc23)c(OC)c1]

Following General Method 9, N1-[(2,4-dimethoxyphenyl)methyl]-N5-[[2-(4-piperidylmethoxy)-4-pyridyl]methyl]isoquinoline-1,5-diamine (75 mg, 0.15 mmol) was reacted with acetone (54 μL, 0.73 mmol) in THF (5 mL). The crude product was purified by flash chromatography (Silica, 0-30% MeOH in DCM) to the product (55 mg, 68% yield) as a pale yellow gum.

[M+H]⁺=556.4

### N5-[[2-[(1-isopropyl-4-piperidyl)methoxy]-4-pyridyl]methyl]isoquinoline-1,5-diamine

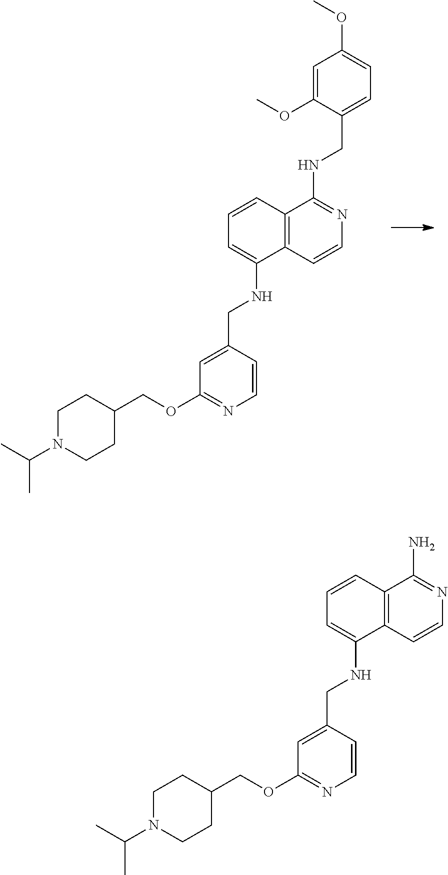 [SMILES: COc1ccc(CNc2nccc3c(NCc4ccnc(OCC5CCN(C(C)C)CC5)c4)cccc23)c(OC)c1 ; CC(C)N1CCC(COc2cc(CNc3cccc4c(N)nccc34)ccn2)CC1]

Deprotection of N1-[(2,4-dimethoxyphenyl)methyl]-N5-[[2-[(1-isopropyl-4-piperidyl)methoxy]-4-pyridyl]methyl]isoquinoline-1,5-diamine (63 mg, 0.11 mmol) was carried out according to General Method 12. The crude product was purified via automated prep HPLC (Mass directed 2-60% over 20 min in basic mobile phase) and lyophilised to afford the product (27 mg, 59% yield) as an off white solid.

[M+H]⁺=406.3

¹H NMR (DMSO, 400 MHz) δ 0.93 (6H, d, J=6.6 Hz), 1.13-1.23 (2H, m), 1.59-1.68 (3H, m), 2.02-2.08 (2H, m), 2.60-2.67 (1H, m), 2.74 (2H, d, J=11.7 Hz), 4.02 (2H, d, J=6.2 Hz), 4.43 (2H, d, J=5.9 Hz), 6.37 (1H, d, J=7.7 Hz), 6.51 (2H, s), 6.71 (1H, s), 6.79 (1H, t, J=6.1 Hz), 6.95 (1H, dd, J=5.4, 0.8 Hz), 7.11 (1H, t, J=7.9 Hz), 7.19 (1H, d, J=6.2 Hz), 7.33 (1H, d, J=8.3 Hz), 7.76 (1H, d, J=6.0 Hz), 8.03 (1H, d, J=5.3 Hz)

### Example Number 4300

### 4-Chloro-N6-[[2-(5,6,7,8-tetrahydroimidazo[1,2-a]pyridin-7-ylmethoxy)-4-pyridyl]methyl]isoquinoline-1,6-diamine

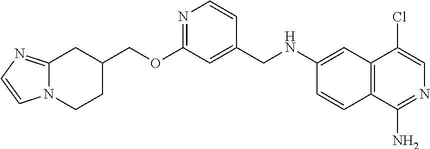 [SMILES: Nc1ncc(Cl)c2cc(NCc3ccnc(OCC4CCn5ccnc5C4)c3)ccc12]

### Methyl N-[4-chloro-6-[[2-(5,6,7,8-tetrahydroimidazo[1,2-a]pyridin-7-ylmethoxy)-4-pyridyl]methylamino]-1-isoquinolyl]carbamate

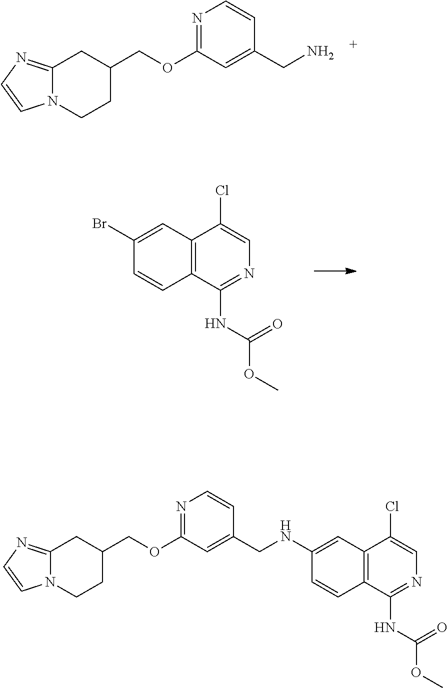 [SMILES: NCc1ccnc(OCC2CCn3ccnc3C2)c1 ; COC(=O)Nc1ncc(Cl)c2cc(Br)ccc12 ; COC(=O)Nc1ncc(Cl)c2cc(NCc3ccnc(OCC4CCn5ccnc5C4)c3)ccc12]

Following General Method 4, [2-(5,6,7,8-tetrahydroimidazo[1,2-a]pyridin-7-ylmethoxy)-4-pyridyl]methanamine (108 mg, 0.42 mmol) was reacted with methyl N-(6-bromo-4-chloro-1-isoquinolyl)carbamate (132 mg, 0.42 mmol) and NaOtBu (121 mg, 1.25 mmol) in THF (6 mL) at rt for 45 min. The mixture was concentrated and purified by flash chromatography (Silica, 0-100% (2% NH₃ in EtOAc/MeCN/EtOH (3:3:1)) in Pet ether 60-80) to afford the product (83 mg, 38% yield) as a pale orange oil.

[M+H]⁺=493.1

### 4-Chloro-N6-[[2-(5,6,7,8-tetrahydroimidazo[1,2-a]pyridin-7-ylmethoxy)-4-pyridyl]methyl]isoquinoline-1,6-diamine

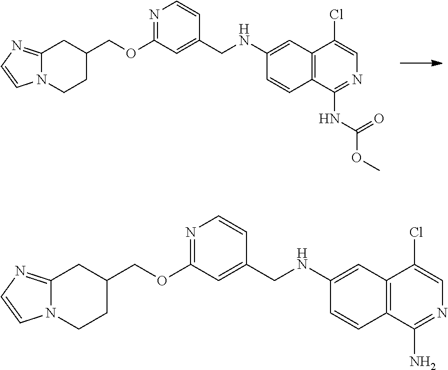 [SMILES: COC(=O)Nc1ncc(Cl)c2cc(NCc3ccnc(OCC4CCn5ccnc5C4)c3)ccc12 ; Nc1ncc(Cl)c2cc(NCc3ccnc(OCC4CCn5ccnc5C4)c3)ccc12]

Deprotection of methyl N-[4-chloro-6-[[2-(5,6,7,8-tetrahydroimidazo[1,2-a]pyridin-7-ylmethoxy)-4-pyridyl]methylamino]-1-isoquinolyl]carbamate (84 mg, 0.16 mmol) was performed following General Method 14a for 18 h. The reaction was cooled to rt and concentrated. Purification was performed by flash chromatography (Silica, 0-100% (2% NH₃ in EtOAc/MeCN/EtOH (3:3:1)) in Pet. Ether 60-80), followed by automated prep HPLC (Mass directed 2-60% over 20 min in basic mobile phase). Lyophilisation afforded the product (25 mg, 37% yield) as a white solid.

[M+H]⁺=435.1

¹H NMR (DMSO-d6, 400 MHz) δ 1.61-1.77 (1H, m), 2.01-2.18 (1H, m), 2.27-2.40 (1H, m), 2.43-2.48 (1H, m), 2.90 (1H, dd, J=16.2, 4.9 Hz), 3.86 (1H, td, J=12.0, 4.8 Hz), 4.06 (1H, ddd, J=12.5, 5.5, 2.8 Hz), 4.24 (2H, d, J=6.5 Hz), 4.42 (2H, d, J=6.1 Hz), 6.57 (2H, s), 6.65 (1H, d, J=2.3 Hz), 6.79 (1H, d, J=1.3 Hz), 6.82 (1H, s), 6.95 (1H, dd, J=9.1, 2.4 Hz), 6.97 (1H, d, J=1.2 Hz), 7.00 (1H, dd, J=5.3, 1.4 Hz), 7.21 (1H, t, J=6.1 Hz), 7.64 (1H, s), 7.94 (1H, d, J=9.1 Hz), 8.09 (1H, dd, J=5.3, 0.7 Hz)

### Example Number 4301

### 4-[[4-[[(1-Amino-5-isoquinolyl)amino]methyl]-2-pyridyl]oxymethyl]-1-methyl-pyridin-2-one

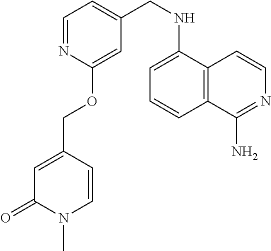 [SMILES: Cn1ccc(COc2cc(CNc3cccc4c(N)nccc34)ccn2)cc1=O]

### 2-[(1-Methyl-2-oxo-4-pyridyl)methoxy]pyridine-4-carbonitrile

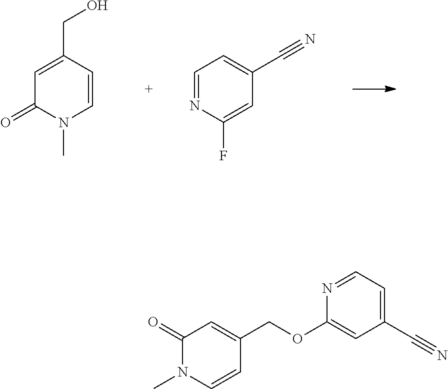

Following General Method 1b, 4-(Hydroxymethyl)-1-methylpyridin-2(1H)-one (100 mg, 0.72 mmol) was reacted with 4-cyano-2-fluoropyridine (88 mg, 0.72 mmol) at 60° C. for 7 days. The reaction mixture was cooled to rt, diluted with water (25 mL) and the product extracted with DCM (3×20 mL). The combined organics were washed with brine (20 mL) and filtered through phase separating paper and concentrated. The crude product was purified by flash chromatography (Silica, 0-20% MeOH in DCM) to afford the product (56 mg, 32% yield).

[M+H]⁺=242.0

¹H NMR (CDCl₃, 400 MHz) δ 3.54 (3H, s), 5.26 (2H, d, J=1.1 Hz), 6.18 (1H, dd, J=6.9, 1.9 Hz), 6.59 (1H, q, J=1.4 Hz), 7.09 (1H, t, J=1.1 Hz), 7.12 (1H, dd, J=5.2, 1.3 Hz), 7.28 (1H, d, J=7.0 Hz), 8.28 (1H, dd, J=5.2, 0.9 Hz)

### 4-[[4-(Aminomethyl)-2-pyridyl]oxymethyl]-1-methyl-pyridin-2-one

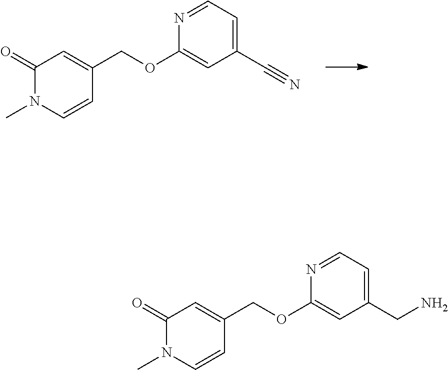 [SMILES: Cn1ccc(COc2cc(C#N)ccn2)cc1=O ; Cn1ccc(COc2cc(CN)ccn2)cc1=O]

Reduction of the nitrile, 2-[(1-methyl-2-oxo-4-pyridyl)methoxy]pyridine-4-carbonitrile (56 mg, 0.23 mmol) was carried out using General Method 3a, using Raney Ni over 15 min. The solvent was removed in vacuo to afford the product (56 mg, 98% yield) as a colourless oil.

[M+H]⁺=246.0

### 4-[[4-[[[1-[(2,4-Dimethoxyphenyl)methylamino]-5-isoquinolyl]amino]methyl]-2-pyridyl]oxymethyl]-1-methyl-pyridin-2-one

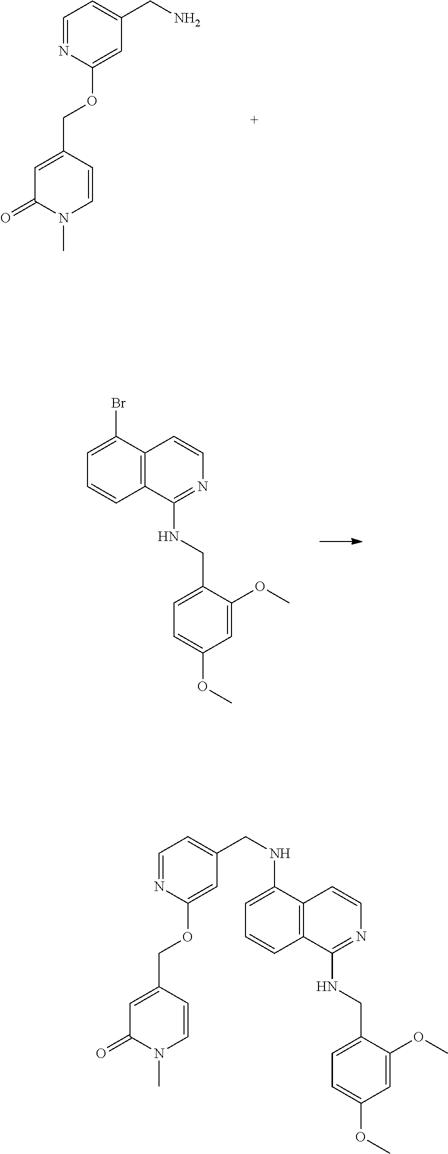 [SMILES: Cn1ccc(COc2cc(CN)ccn2)cc1=O ; COc1ccc(CNc2nccc3c(Br)cccc23)c(OC)c1 ; COc1ccc(CNc2nccc3c(NCc4ccnc(OCc5ccn(C)c(=O)c5)c4)cccc23)c(OC)c1]

Following General Method 4, 5-bromo-N-[(2,4-dimethoxyphenyl)methyl]isoquinolin-1-amine (87 mg, 0.23 mmol) was reacted with 4-[[4-(aminomethyl)-2-pyridyl]oxymethyl]-1-methyl-pyridin-2-one (57 mg, 0.23 mmol) Cs₂CO₃ (152 mg, 0.46 mmol) in 1,4-dioxane (5 mL) at 60° C. for 20 h. After quenching and filtering through Celite®, the residue was purified by flash chromatography (Silica, 20-100% EtOAc in Pet. Ether followed by 0-20% MeOH in EtOAc) to afford the product (102 mg, 82% yield) as an orange glass.

[M+H]⁺=538.2

### 4-[[4-[[(1-amino-5-isoquinolyl)amino]methyl]-2-pyridyl]oxymethyl]-1-methyl-pyridin-2-one

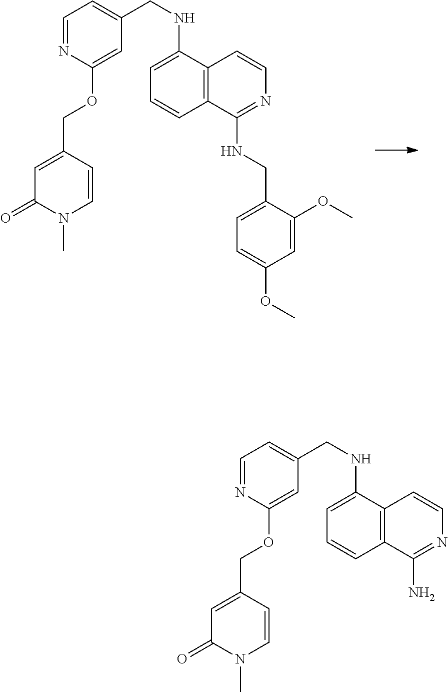 [SMILES: COc1ccc(CNc2nccc3c(NCc4ccnc(OCc5ccn(C)c(=O)c5)c4)cccc23)c(OC)c1 ; Cn1ccc(COc2cc(CNc3cccc4c(N)nccc34)ccn2)cc1=O]

Deprotection of 4-[[4-[[[1-[(2,4-dimethoxyphenyl)methylamino]-5-isoquinolyl]amino]methyl]-2-pyridyl]oxymethyl]-1-methyl-pyridin-2-one (102 mg, 0.19 mmol) was carried out using General Method 12. The product was purified via automated prep HPLC (Mass directed 2-60% over 20 min in basic mobile phase) and lyophilised to afford the product (25 mg, 34% yield) as an off-white solid.

[M+H]⁺=388.2

¹H NMR (DMSO, 400 MHz) δ 3.37 (3H, s), 4.47 (2H, d, J=6.0 Hz), 5.17 (2H, d, J=1.2 Hz), 6.18 (1H, dd, J=6.9, 1.9 Hz), 6.29 (1H, d, J=1.7 Hz), 6.40 (1H, d, J=7.7 Hz), 6.52 (2H, s), 6.82 (1H, t, J=6.1 Hz), 6.87 (1H, s), 7.01 (1H, dd, J=5.3, 1.4 Hz), 7.12 (1H, t, J=8.0 Hz), 7.20 (1H, d, J=6.1 Hz), 7.34 (1H, d, J=8.3 Hz), 7.62 (1H, d, J=7.0 Hz), 7.77 (1H, d, J=6.0 Hz), 8.05 (1H, d, J=5.4 Hz)

### Example Number 1044

### 4-Chloro-N6-[[6-(5,6,7,8-tetrahydroimidazo[1,2-a]pyridin-7-ylmethylamino)-3-pyridyl]methyl]isoquinoline-1,6-diamine

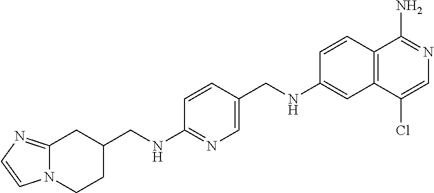 [SMILES: Nc1ncc(Cl)c2cc(NCc3ccc(NCC4CCn5ccnc5C4)nc3)ccc12]

### 6-(5,6,7,8-tetrahydroimidazo[1,2-a]pyridin-7-ylmethylamino)pyridine-3-carbonitrile

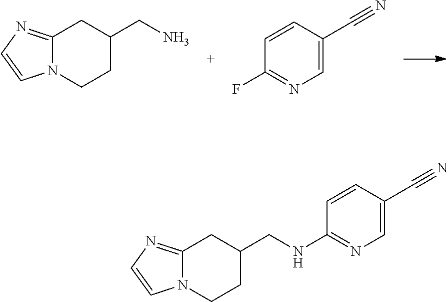 [SMILES: NCC1CCn2ccnc2C1 ; N#Cc1ccc(F)nc1 ; N#Cc1ccc(NCC2CCn3ccnc3C2)nc1]

Following General Method 1d, 5,6,7,8-tetrahydroimidazo[1,2-a]pyridin-7-ylmethanamine (100 mg, 0.66 mmol) was reacted with 5-cyano-2-fluoropyridine (81 mg, 0.66 mmol) at 90° C. for 90 min. The crude material was purified via flash chromatography (Silica, 0-20% MeOH in DCM) to give the product (100 mg, 60% yield) as an off white solid.

[M+H]⁺=254.1

### 5-(Aminomethyl)-N-(5,6,7,8-tetrahydroimidazo[1,2-a]pyridin-7-ylmethyl)pyridin-2-amine

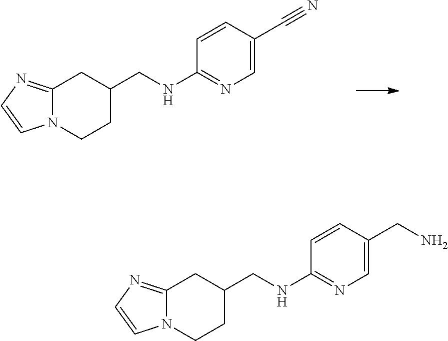 [SMILES: N#Cc1ccc(NCC2CCn3ccnc3C2)nc1 ; NCc1ccc(NCC2CCn3ccnc3C2)nc1]

Reduction of the nitrile, 6-(5,6,7,8-tetrahydroimidazo[1,2-a]pyridin-7-ylmethylamino)pyridine-3-carbonitrile (165 mg, 0.65 mmol) was carried out using General Method 3a, using Raney Ni over 45 min. The solvent was removed in vacuo to afford the product (147 mg, 88% yield) as a yellow oil.

[M+H]⁺=258.1

### Methyl N-[4-chloro-6-[[6-(5,6,7,8-tetrahydroimidazo[1,2-a]pyridin-7-ylmethylamino)-3-pyridyl]methylamino]-1-isoquinolyl]carbamate

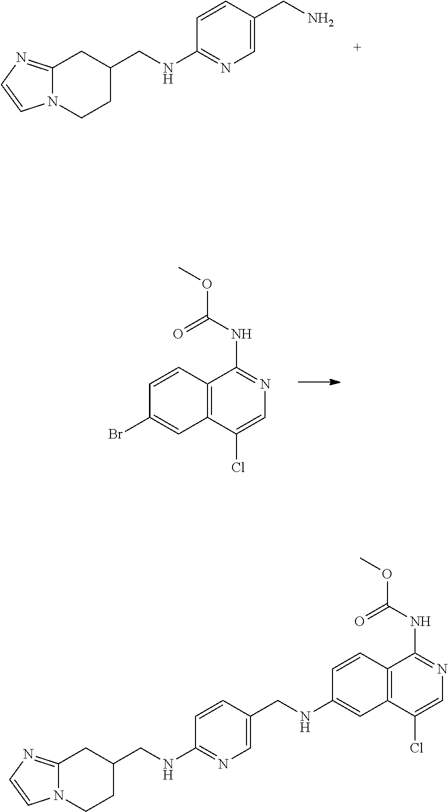 [SMILES: NCc1ccc(NCC2CCn3ccnc3C2)nc1 ; COC(=O)Nc1ncc(Cl)c2cc(Br)ccc12 ; COC(=O)Nc1ncc(Cl)c2cc(NCc3ccc(NCC4CCn5ccnc5C4)nc3)ccc12]

Following General Method 4, 5-(aminomethyl)-N-(5,6,7,8-tetrahydroimidazo[1,2-a]pyridin-7-ylmethyl)pyridin-2-amine (147 mg, 0.57 mmol) was reacted with methyl N-(6-bromo-4-chloro-1-isoquinolyl)carbamate (180 mg, 0.57 mmol) and NaOtBu (110 mg, 1.14 mmol) in THF (5 mL) at 40° C. for 5 h. After quenching and filtering through Celite®, the residue was purified by flash chromatography (Silica, 0-100% (2% NH₃ in EtOAc/MeCN/EtOH (3:3:1)) in Pet. Ether 60-80) to afford the product (133 mg, 47% yield) as a pale yellow gum.

[M+H]⁺=492.2

### 4-Chloro-N6-[[6-(5,6,7,8-tetrahydroimidazo[1,2-a]pyridin-7-ylmethylamino)-3-pyridyl]methyl]isoquinoline-1,6-diamine

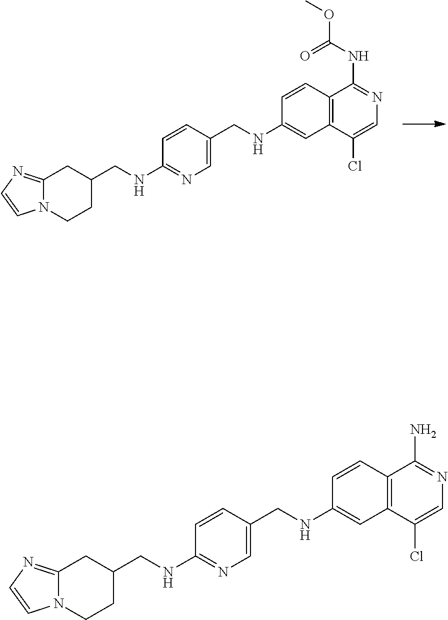 [SMILES: COC(=O)Nc1ncc(Cl)c2cc(NCc3ccc(NCC4CCn5ccnc5C4)nc3)ccc12 ; Nc1ncc(Cl)c2cc(NCc3ccc(NCC4CCn5ccnc5C4)nc3)ccc12]

Deprotection of methyl N-[4-chloro-6-[[6-(5,6,7,8-tetrahydroimidazo[1,2-a]pyridin-7-ylmethylamino)-3-pyridyl]methylamino]-1-isoquinolyl]carbamate (133 mg, 0.27 mmol) was performed using General Method 14 over 24 h. The reaction was cooled and concentrated. The residue was purified via automated prep HPLC (Mass directed 2-60% over 20 min in basic mobile phase) and lyophilised to afford the product (40 mg, 34% yield) as an off white solid.

[M+H]⁺=434.1

¹H NMR (DMSO, 400 MHz) δ 1.54-1.69 (1H, m), 2.05 (1H, d, J=13.5 Hz), 2.11-2.19 (1H, m), 2.37 (1H, dd, J=16.4, 10.6 Hz), 2.88 (1H, dd, J=16.4, 5.1, 1.5 Hz), 3.22-3.31 (2H, m), 3.81 (1H, td, J=12.3, 11.8, 4.7 Hz), 4.00-4.10 (1H, m), 4.17 (2H, d, J=5.4 Hz), 6.50 (1H, d, J=8.5, 0.7 Hz), 6.54 (2H, s), 6.67 (1H, t, J=5.8 Hz), 6.71 (1H, d, J=2.3 Hz), 6.78 (1H, d, J=1.2 Hz), 6.86-6.98 (3H, m), 7.41 (1H, dd, J=8.6, 2.4 Hz), 7.65 (1H, s), 7.90 (1H, d, J=9.1 Hz), 8.01 (1H, d, J=2.3 Hz)

### Example Number 1131

### 2-[(3S)-1-[5-[[(1-Amino-5-isoquinolyl)amino]methyl]-2-pyridyl]pyrrolidin-3-yl]propan-2-ol

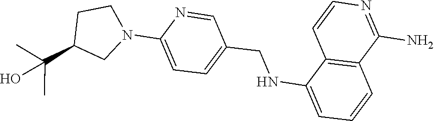 [SMILES: CC(C)(O)[C@H]1CCN(c2ccc(CNc3cccc4c(N)nccc34)cn2)C1]

### (S)-6-(3-(2-Hydroxypropan-2-yl)pyrrolidin-1-yl)nicotinonitrile

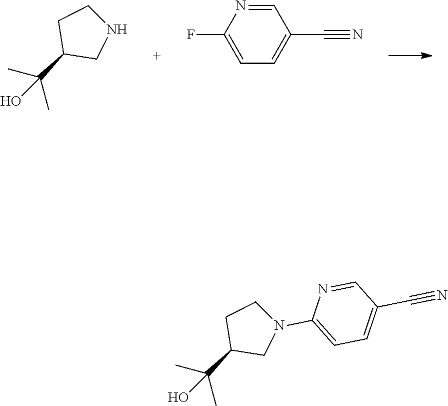

Following General Method 1d, (S)-2-(3-pyrrolidinyl)-2-propanol (106 mg, 0.82 mmol) was reacted with and 5-cyano-2-fluoropyridine (100 mg, 0.82 mmol) at 120° C. for 60 min under microwave irradiation. The product was isolated (199 mg, 98% yield) and used without further purification.

[M+H]⁺=232.1

¹H NMR (CDCl₃, 400 MHz) δ 1.31 (3H, s), 1.31 (3H, s), 1.37 (1H, s), 1.97 (1H, d, J=12.8 Hz), 2.05-2.16 (1H, m), 2.39 (1H, q, J=9.0 Hz), 3.40 (2H, dt, J=20.8, 10.2 Hz), 3.69 (2H, s), 6.34 (1H, dd, J=8.9, 0.8 Hz), 7.57 (1H, dd, J=8.9, 2.3 Hz), 8.40 (1H, dd, J=2.3, 0.8 Hz)

### 2-[(3S)-1-[5-(Aminomethyl)-2-pyridyl]pyrrolidin-3-yl]propan-2-ol

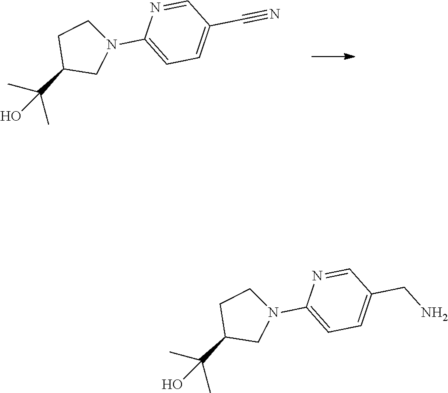 [SMILES: CC(C)(O)[C@H]1CCN(c2ccc(C#N)cn2)C1 ; CC(C)(O)[C@H]1CCN(c2ccc(CN)cn2)C1]

The nitrile, (S)-6-(3-(2-hydroxypropan-2-yl)pyrrolidin-1-yl)nicotinonitrile (199 mg, 0.81 mmol) was reduced according to General Method 3a, using Raney Ni over 30 min. The solvent was removed in vacuo to the product (190 mg, quantitative yield) as a colourless oil.

[M+H]⁺=236.1

### 2-[(3S)-1-[5-[[[1-[(2,4-Dimethoxyphenyl)methylamino]-5-isoquinolyl]amino]methyl]-2-pyridyl]pyrrolidin-3-yl]propan-2-ol

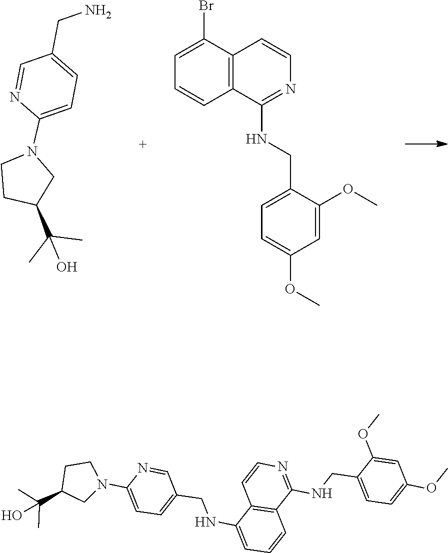 [SMILES: CC(C)(O)[C@H]1CCN(c2ccc(CN)cn2)C1 ; COc1ccc(CNc2nccc3c(Br)cccc23)c(OC)c1 ; COc1ccc(CNc2nccc3c(NCc4ccc(N5CC[C@H](C(C)(C)O)C5)nc4)cccc23)c(OC)c1]

Using General Method 4, 5-bromo-N-[(2,4-dimethoxyphenyl)methyl]isoquinolin-1-amine (151 mg, 0.4 mmol) was reacted with 2-[(3S)-1-[5-(aminomethyl)-2-pyridyl]pyrrolidin-3-yl]propan-2-ol (95 mg, 0.4 mmol) and Cs₂CO₃ (265 mg, 0.81 mmol) in 1,4-dioxane (5 mL) at 60° C. for 20 h. After quenching and filtering through Celite®, the crude product was purified by flash chromatography (Silica, 0-20% MeOH in EtOAc) to afford the product (73 mg, 34% yield) as a colourless glass.

[M+H]⁺=528.3

### 2-[(3S)-1-[5-[[(1-Amino-5-isoquinolyl)amino]methyl]-2-pyridyl]pyrrolidin-3-yl]propan-2-ol

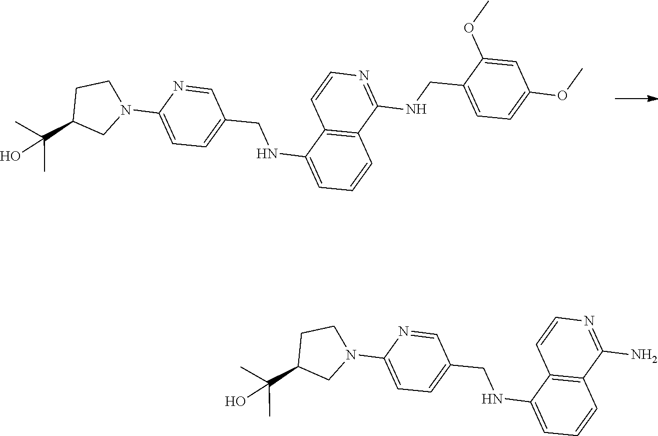

Using General Method 12, 2-[(3S)-1-[5-[[[1-[(2,4-dimethoxyphenyl)methylamino]-5-isoquinolyl]amino]methyl]-2-pyridyl]pyrrolidin-3-yl]propan-2-ol (73 mg, 0.14 mmol) was deprotected. The product was purified via automated prep HPLC (Mass directed 2-60% over 20 min in basic mobile phase) and lyophilised to afford the product (17 mg, 33% yield) as an off-white solid.

[M+H]⁺=378.3

¹H NMR (DMSO, 400 MHz) δ 1.11 (3H, s), 1.12 (3H, s), 1.75-1.94 (3H, m), 2.24 (1H, p, J=8.7 Hz), 3.13-3.26 (2H, m), 3.50 (2H, td, J=8.8, 8.2, 4.7 Hz), 4.28 (2H, d, J=5.7 Hz), 6.36 (1H, d, J=8.6 Hz), 6.49 (2H, s), 6.53 (1H, s), 6.56 (1H, q, J=4.9, 4.0 Hz), 7.12 (1H, d, J=8.0 Hz), 7.16 (1H, d, J=5.8 Hz), 7.30 (1H, d, J=8.3 Hz), 7.48 (1H, dd, J=8.6, 2.4 Hz), 7.72 (1H, d, J=6.1 Hz), 8.08 (1H, d, J=2.3 Hz)

### Example Number 1052

### 4-Chloro-N6-[[2-(5,6,7,8-tetrahydroimidazo[1,2-a]pyridin-7-ylmethoxy)pyrimidin-5-yl]methyl]isoquinoline-1,6-diamine

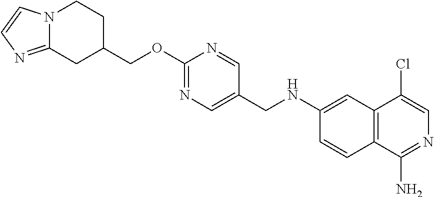 [SMILES: Nc1ncc(Cl)c2cc(NCc3cnc(OCC4CCn5ccnc5C4)nc3)ccc12]

### 2-(5,6,7,8-Tetrahydroimidazo[1,2-a]pyridin-7-ylmethoxy)pyrimidine-5-carbonitrile

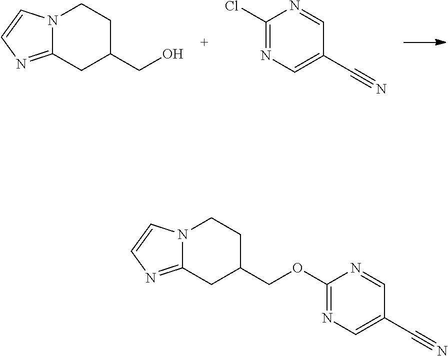 [SMILES: OCC1CCn2ccnc2C1 ; N#Cc1cnc(Cl)nc1 ; N#Cc1cnc(OCC2CCn3ccnc3C2)nc1]

Following General Method 1a, 5,6,7,8-tetrahydroimidazo[1,2-a]pyridin-7-ylmethanol (200 mg, 1.31 mmol) was reacted with 2-Chloro-5-pyrimidinecarbonitrile (183 mg, 1.31 mmol) in THF for 18 h. The crude product was purified by flash chromatography (Silica, 0-20% MeOH in DCM) to afford the product (90 mg, 27% yield) as a brown solid.

[M+H]⁺=256.0

### [2-(5,6,7,8-Tetrahydroimidazo[1,2-a]pyridin-7-ylmethoxy)pyrimidin-5-yl]methanamine

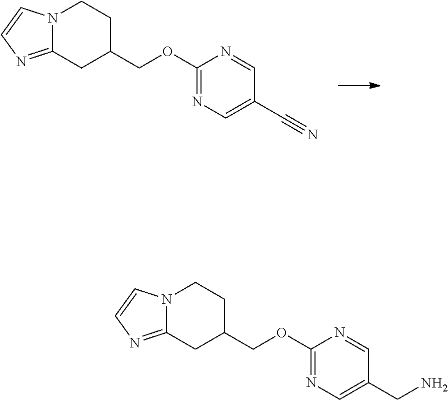 [SMILES: N#Cc1cnc(OCC2CCn3ccnc3C2)nc1 ; NCc1cnc(OCC2CCn3ccnc3C2)nc1]

Reduction of the nitrile, 2-(5,6,7,8-tetrahydroimidazo[1,2-a]pyridin-7-ylmethoxy)pyrimidine-5-carbonitrile (90 mg, 0.35 mmol) was carried out using General method 3a, using Raney Ni over 15 min. The solvent was removed in vacuo to the product (100 mg, quantitative yield) as a yellow oil.

[M+H]⁺=260.1

### Methyl N-[4-chloro-6-[[2-(5,6,7,8-tetrahydroimidazo[1,2-a]pyridin-7-ylmethoxy)pyrimidin-5-yl]methylamino]-1-isoquinolyl]carbamate

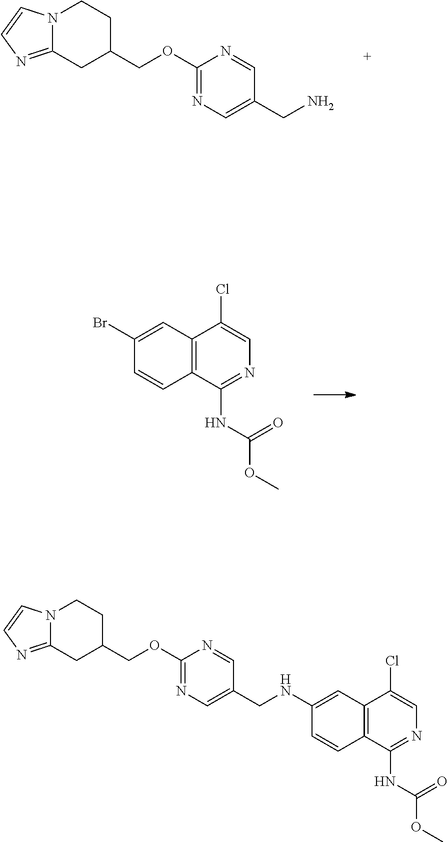 [SMILES: NCc1cnc(OCC2CCn3ccnc3C2)nc1 ; COC(=O)Nc1ncc(Cl)c2cc(Br)ccc12 ; COC(=O)Nc1ncc(Cl)c2cc(NCc3cnc(OCC4CCn5ccnc5C4)nc3)ccc12]

Using General Method 4, [2-(5,6,7,8-tetrahydroimidazo[1,2-a]pyridin-7-ylmethoxy)pyrimidin-5-yl]methanamine (100 mg, 0.39 mmol) was reacted with methyl N-(6-bromo-4-chloro-1-isoquinolyl)carbamate (122 mg, 0.39 mmol) and NaOtBu (111 mg, 1.16 mmol) in THF (5 mL) at 40° C. for 1 h. The mixture was concentrated and purified by flash chromatography (Silica, 0-100% (2% NH₃ in EtOAc/MeCN/EtOH (3:3:1)) in Pet ether 60-80) to afford the product (91 mg, 37% yield) as a pale yellow oil.

[M+H]⁺=494.2

### 4-Chloro-N6-[[2-(5,6,7,8-tetrahydroimidazo[1,2-a]pyridin-7-ylmethoxy)pyrimidin-5-yl]methyl]isoquinoline-1,6-diamine

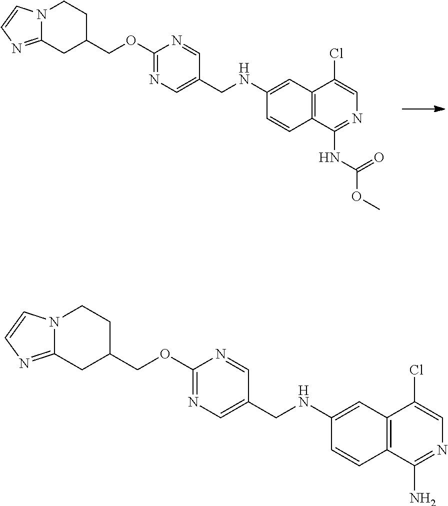 [SMILES: COC(=O)Nc1ncc(Cl)c2cc(NCc3cnc(OCC4CCn5ccnc5C4)nc3)ccc12 ; Nc1ncc(Cl)c2cc(NCc3cnc(OCC4CCn5ccnc5C4)nc3)ccc12]

Deprotection of methyl N-[4-chloro-6-[[2-(5,6,7,8-tetrahydroimidazo[1,2-a]pyridin-7-ylmethoxy)pyrimidin-5-yl]methylamino]-1-isoquinolyl]carbamate (91 mg, 0.18 mmol) was carried out using General Method 14b at 60° C. for 4 days. The crude product was purified via automated prep HPLC (Mass directed 2-60% over 20 min in basic mobile phase) and lyophilised to afford the product (8 mg, 10% yield) as a white solid.

[M+H]⁺=436.1

¹H NMR (DMSO-d6, 400 MHz) δ 1.65-1.81 (1H, m), 2.05-2.19 (1H, m), 2.33-2.44 (1H, m), 2.86-2.97 (1H, m), 3.88 (1H, td, J=11.9, 4.7 Hz), 4.02-4.14 (1H, m), 4.30 (2H, dd, J=6.5, 1.5 Hz), 4.37 (2H, d, J=5.6 Hz), 6.58 (2H, br s), 6.73 (1H, d, J=2.3 Hz), 6.80 (1H, d, J=1.3 Hz), 6.96 (1H, dd, J=9.1, 2.4 Hz), 6.98 (1H, d, J=1.3 Hz), 7.04 (1H, t, J=5.7 Hz), 7.67 (1H, s), 7.94 (1H, d, J=9.0 Hz), 8.65 (2H, s)

### Example Number 4320

### N5-[[2-[(3-methyl-5,6,7,8-tetrahydroimidazo[1,2-a]pyridin-7-yl)methoxy]-4-pyridyl]methyl]isoquinoline-1,5-diamine

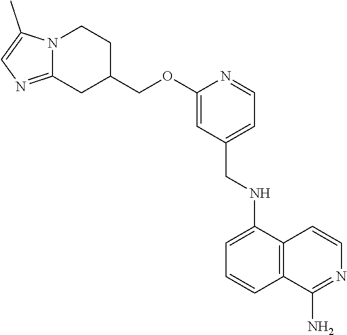 [SMILES: Cc1cnc2n1CCC(COc1cc(CNc3cccc4c(N)nccc34)ccn1)C2]

### 2-[(3-Methyl-5,6,7,8-tetrahydroimidazo[1,2-a]pyridin-7-yl)methoxy]pyridine-4-carbonitrile

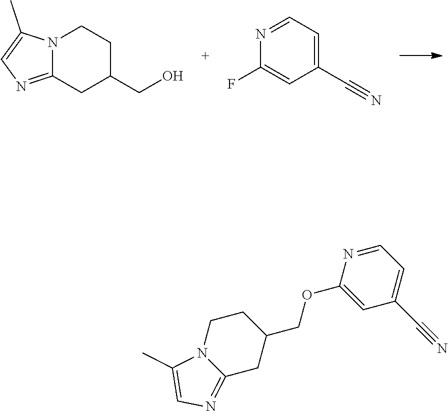 [SMILES: Cc1cnc2n1CCC(CO)C2 ; N#Cc1ccnc(F)c1 ; Cc1cnc2n1CCC(COc1cc(C#N)ccn1)C2]

Following General Method 1d, (3-methyl-5,6,7,8-tetrahydroimidazo[1,2-a]pyridin-7-yl)methanol (130 mg, 0.78 mmol) was reacted with 4-cyano-2-fluoropyridine (105 mg, 0.86 mmol) at 60° C. for 18 h. The reaction mixture was cooled to rt and diluted with water (5 mL). The crude product was extracted into EtOAc (3×20 mL), dried (MgSO₄), filtered and concentrated. The crude product was purified by flash chromatography (Silica, 0-20% MeOH in DCM) to afford the product (93 mg, 44% yield) as a brown oil.

[M+H]⁺=269.0

### [2-[(3-methyl-5,6,7,8-tetrahydroimidazo[1,2-a]pyridin-7-yl)methoxy]-4-pyridyl]methanamine

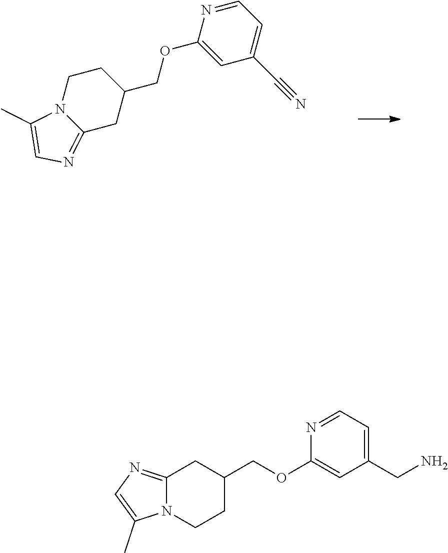 [SMILES: Cc1cnc2n1CCC(COc1cc(C#N)ccn1)C2 ; Cc1cnc2n1CCC(COc1cc(CN)ccn1)C2]

The nitrile, 2-(5,6,7,8-tetrahydroimidazo[1,2-a]pyridin-7-ylmethoxy)pyrimidine-5-carbonitrile (176 mg, 0.69 mmol) was reduced according to General Method 3a, using Raney Ni over 30 min. The solvent was removed in vacuo to deliver the product (91 mg, 96% yield) as a yellow oil.

[M+H]⁺=273.1

### N1-[(2,4-dimethoxyphenyl)methyl]-N5-[[2-[(2-methyl-5,6,7,8-tetrahydroimidazo[1,2-a]pyridin-7-yl)methoxy]-4-pyridyl]methyl]isoquinoline-1,5-diamine

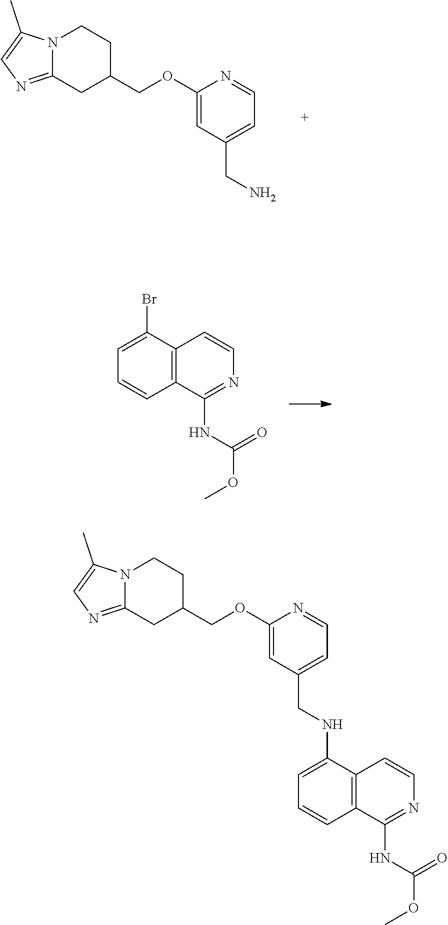 [SMILES: Cc1cnc2n1CCC(COc1cc(CN)ccn1)C2 ; COC(=O)Nc1nccc2c(Br)cccc12 ; COC(=O)Nc1nccc2c(NCc3ccnc(OCC4CCn5c(C)cnc5C4)c3)cccc12]

Following General Method 4, 5-bromo-N-[(2,4-dimethoxyphenyl)methyl]isoquinolin-1-amine (125 mg, 0.33 mmol) was reacted with [2-[(3-methyl-5,6,7,8-tetrahydroimidazo[1,2-a]pyridin-7-yl)methoxy]-4-pyridyl]methanamine (91 mg, 0.33 mmol) and NaOtBu (75 mg, 0.67 mmol) in 1,4-dioxane (5 mL) 60° C. for 1 h. After quenching and filtering through Celite®, the crude product was purified via flash chromatography (Silica, 0-20% (10% NH₄OH in MeOH) in DCM) to afford the product (111 mg, 59% yield) as an orange solid.

[M+H]⁺=565.3

### N5-[[2-[(3-methyl-5,6,7,8-tetrahydroimidazo[1,2-a]pyridin-7-yl)methoxy]-4-pyridyl]methyl]isoquinoline-1,5-diamine

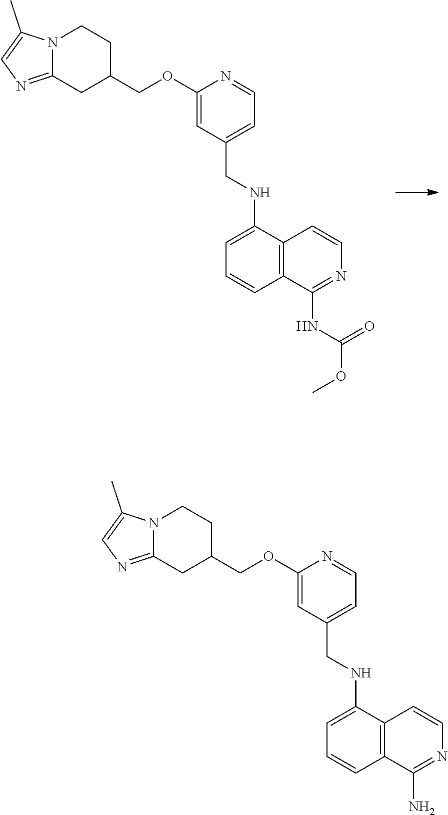 [SMILES: COC(=O)Nc1nccc2c(NCc3ccnc(OCC4CCn5c(C)cnc5C4)c3)cccc12 ; Cc1cnc2n1CCC(COc1cc(CNc3cccc4c(N)nccc34)ccn1)C2]

Deprotection of N1-[(2,4-dimethoxyphenyl)methyl]-N5-[[2-[(3-methyl-5,6,7,8-tetrahydroimidazo[1,2-a]pyridin-7-yl)methoxy]-4-pyridyl]methyl]isoquinoline-1,5-diamine (111 mg, 0.2 mmol) was carried out using General Method 12. Purification was performed via automated prep HPLC (Mass directed 2-60% over 20 min in basic mobile phase) and the product was lyophilised to the product (31 mg, 38% yield) as an off white solid.

[M+H]⁺=415.2

¹H NMR (DMSO, 400 MHz) δ 1.60-1.78 (1H, m), 2.08 (3H, s), 2.09-2.19 (1H, m), 2.21-2.34 (1H, m), 2.43 (1H, dd, J=16.1, 10.8 Hz), 2.84 (1H, dd, J=16.1, 4.9, 1.5 Hz), 3.66 (1H, td, J=11.8, 4.9 Hz), 3.85-3.95 (1H, m), 4.15-4.27 (2H, m), 4.45 (2H, d, J=5.9 Hz), 6.38 (1H, d, J=7.6 Hz), 6.50 (1H, d, J=1.2 Hz), 6.54 (2H, s), 6.74-6.80 (1H, m), 6.83 (1H, t, J=6.1 Hz), 6.98 (1H, dd, J=5.3, 1.4 Hz), 7.12 (1H, t, J=8.0 Hz), 7.20 (1H, d, J=6.1 Hz), 7.33 (1H, d, J=8.3 Hz), 7.77 (1H, d, J=6.0 Hz), 8.06 (1H, d, J=5.3 Hz)

### Example Number 4429

### N6-[[2-(5,6,7,8-tetrahydroimidazo[1,2-a]pyridin-7-ylmethoxy)-4-pyridyl]methyl]-2,7-naphthyridine-1,6-diamine

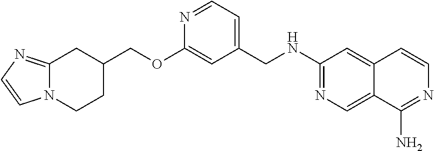 [SMILES: Nc1nccc2cc(NCc3ccnc(OCC4CCn5ccnc5C4)c3)ncc12]

### N1-[(2,4-dimethoxyphenyl)methyl]-N6-[[2-(5,6,7,8-tetrahydroimidazo[1,2-a]pyridin-7-ylmethoxy)-4-pyridyl]methyl]-2,7-naphthyridine-1,6-diamine

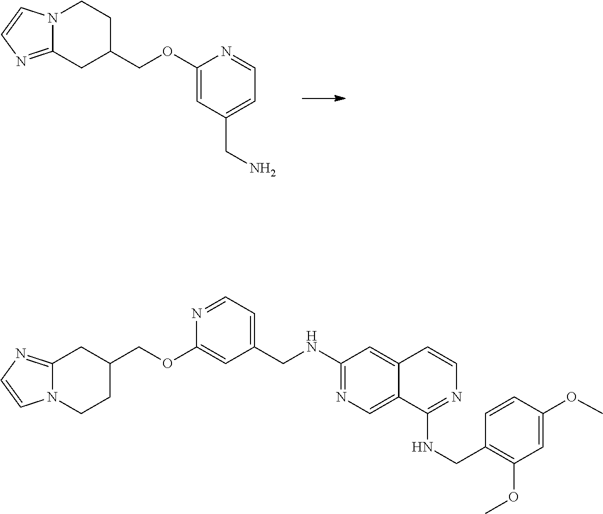 [SMILES: NCc1ccnc(OCC2CCn3ccnc3C2)c1 ; COc1ccc(CNc2nccc3cc(NCc4ccnc(OCC5CCn6ccnc6C5)c4)ncc23)c(OC)c1]

Following General Method 4, 6-chloro-N-[(2,4-dimethoxyphenyl)methyl]-2,7-naphthyridin-1-amine (127 mg, 0.38 mmol) was reacted with [2-(5,6,7,8-tetrahydroimidazo[1,2-a]pyridin-7-ylmethoxy)-4-pyridyl]methanamine (68 mg, 0.26 mmol) and Cs₂CO₃ (216 mg, 0.66 mmol) in THF (3 mL) at 60° C. for 48 h. The reaction mixture was cooled to rt and concentrated before purification by flash chromatography (Silica, 0-100% (2% NH₃ in EtOAc/MeCN/EtOH (3:3:1)) in Pet. Ether) to afford the product (120 mg, 82% yield) as a pale yellow oil.

[M+H]⁺=552.3

### N6-[[2-(5,6,7,8-tetrahydroimidazo[1,2-a]pyridin-7-ylmethoxy)-4-pyridyl]methyl]-2,7-naphthyridine-1,6-diamine

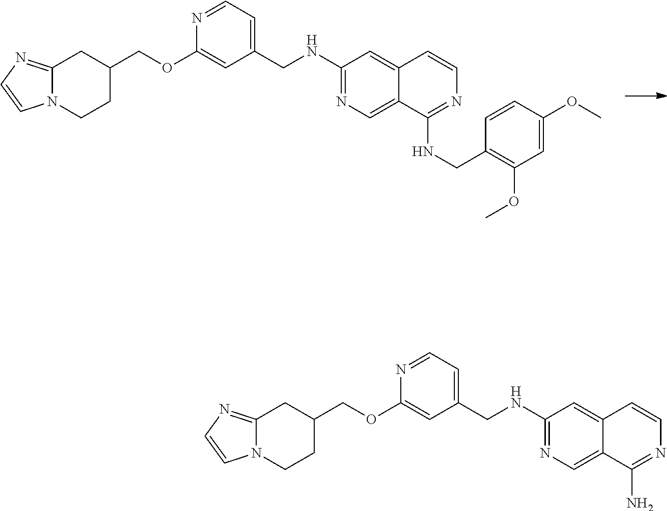 [SMILES: COc1ccc(CNc2nccc3cc(NCc4ccnc(OCC5CCn6ccnc6C5)c4)ncc23)c(OC)c1 ; Nc1nccc2cc(NCc3ccnc(OCC4CCn5ccnc5C4)c3)ncc12]

Deprotection of N1-[(2,4-dimethoxyphenyl)methyl]-N6-[[2-(5,6,7,8-tetrahydroimidazo[1,2-a]pyridin-7-ylmethoxy)-4-pyridyl]methyl]-2,7-naphthyridine-1,6-diamine (120 mg, 0.22 mmol) was carried out following General Method 12, over 3 h. The crude product was purified via automated prep HPLC. (Mass directed 2-60% over 20 min in basic mobile phase) and lyophilised to the product (22 mg, 26% yield) as an off-white solid.

[M+H]⁺=402.2

¹H NMR (DMSO-d₆, 400 MHz) δ 1.62-1.79 (1H, m), 2.10 (1H, d, J=13.8 Hz), 2.27-2.41 (1H, m), 2.42-2.48 (1H, m), 2.90 (1H, dd, J=16.2, 4.8 Hz), 3.85 (1H, dt, J=12.0, 5.8 Hz), 4.00-4.11 (1H, m), 4.23 (2H, d, J=6.5 Hz), 4.50 (2H, d, J=6.3 Hz), 6.33 (1H, s), 6.47 (1H, d, J=5.8 Hz), 6.75 (1H, s), 6.79 (1H, d, J=1.3 Hz), 6.82 (2H, s), 6.95 (1H, dd, J=5.3, 1.4 Hz), 6.97 (1H, d, J=1.3 Hz), 7.36 (1H, t, J=6.3 Hz), 7.62 (1H, d, J=5.9 Hz), 8.05 (1H, d, J=5.2 Hz), 9.05 (1H, s) ppm.

### Example Number 1049

### N5-[[6-(5,6,7,8-tetrahydroimidazo[1,2-a]pyridin-7-yloxy)-3-pyridyl]methyl]isoquinoline-1,5-diamine

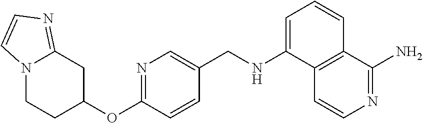 [SMILES: Nc1nccc2c(NCc3ccc(OC4CCn5ccnc5C4)nc3)cccc12]

### 6-(5,6,7,8-Tetrahydroimidazo[1,2-a]pyridin-7-yloxy)pyridine-3-carbonitrile

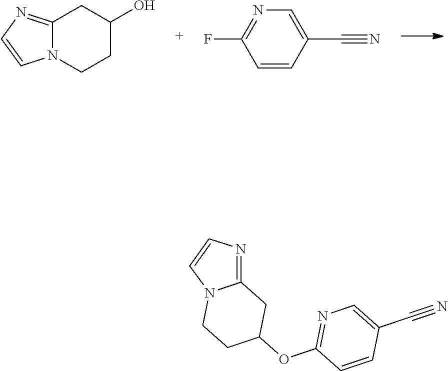 [SMILES: OC1CCn2ccnc2C1 ; N#Cc1ccc(F)nc1 ; N#Cc1ccc(OC2CCn3ccnc3C2)nc1]

Following General Method 1b, 5,6,7,8-Tetrahydroimidazo[1,2-a]pyridin-7-ol (100 mg, 0.72 mmol) was reacted with 5-cyano-2-fluoropyridine (88 mg, 0.72 mmol) in MeCN (5 mL) at 60° C. for 5 h. The reaction mixture was cooled to rt and diluted with water (5 mL). The crude product was extracted into DCM (3×20 mL), dried (MgSO₄) and concentrated. The crude product was purified by flash chromatography (Silica, 0-20% MeOH in EtOAc) to afford the product (68 mg, 39% yield) as an orange glass.

[M+H]⁺=241.1

¹H NMR (CDCl₃, 400 MHz) δ 2.31 (1H, dddd, J=14.0, 8.5, 5.8, 2.5 Hz), 2.40 (1H, ddtd, J=13.1, 6.5, 5.2, 1.2 Hz), 3.17-3.33 (2H, m), 4.03-4.20 (2H, m), 5.74 (1H, dtd, J=7.2, 4.8, 2.5 Hz), 6.79 (1H, dd, J=8.7, 0.8 Hz), 6.86 (1H, d, J=1.3 Hz), 7.03 (1H, d, J=1.3 Hz), 7.80 (1H, dd, J=8.7, 2.3 Hz), 8.48 (1H, dd, J=2.4, 0.8 Hz)

### [6-(5,6,7,8-Tetrahydroimidazo[1,2-a]pyridin-7-yloxy)-3-pyridyl]methanamine

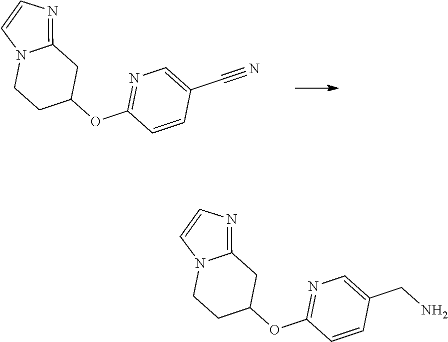 [SMILES: N#Cc1ccc(OC2CCn3ccnc3C2)nc1 ; NCc1ccc(OC2CCn3ccnc3C2)nc1]

The nitrile, 6-(5,6,7,8-tetrahydroimidazo[1,2-a]pyridin-7-yloxy)pyridine-3-carbonitrile (68 mg, 0.28 mmol) was reduced using General Method 3a using Raney Ni over 30 min. The solvent was removed in vacuo to afford the product (66 mg, 95% yield) as a pale yellow oil.

[M+H]⁺=245.1

### N1-[(2,4-Dimethoxyphenyl)methyl]-N5-[[6-(5,6,7,8-tetrahydroimidazo[1,2-a]pyridin-7-yloxy)-3-pyridyl]methyl]isoquinoline-1,5-diamine

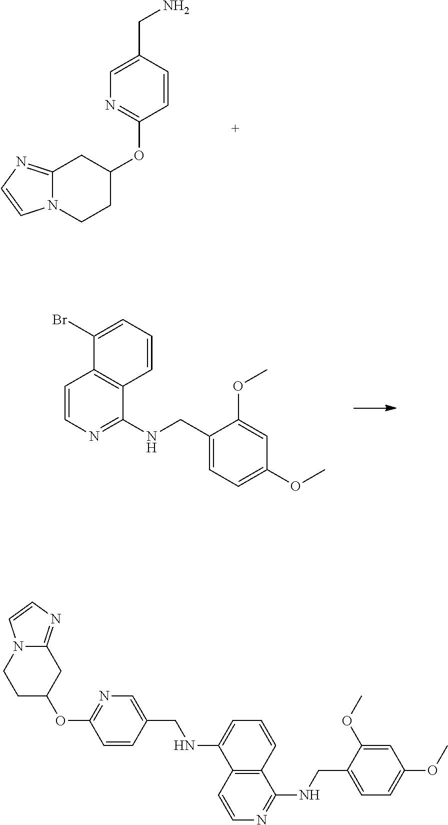 [SMILES: NCc1ccc(OC2CCn3ccnc3C2)nc1 ; COc1ccc(CNc2nccc3c(Br)cccc23)c(OC)c1 ; COc1ccc(CNc2nccc3c(NCc4ccc(OC5CCn6ccnc6C5)nc4)cccc23)c(OC)c1]

Using General Method 4, [6-(5,6,7,8-tetrahydroimidazo[1,2-a]pyridin-7-yloxy)-3-pyridyl]methanamine (66 mg, 0.27 mmol) was reacted with 5-bromo-N-[(2,4-dimethoxyphenyl)methyl]isoquinolin-1-amine (101 mg, 0.27 mmol) and Cs₂CO₃ (177 mg, 0.54 mmol) in 1,4-dioxane (5 mL) at 60° C. for 24 h. The reaction was cooled to rt, quenched and filtered through Celite®. The crude product was purified by flash chromatography (Silica, 0-30% MeOH in EtOAc) to afford the product (52 mg, 36% yield) as a colourless glass.

[M+H]⁺=537.3

### N5-[[6-(5,6,7,8-tetrahydroimidazo[1,2-a]pyridin-7-yloxy)-3-pyridyl]methyl]isoquinoline-1,5-diamine

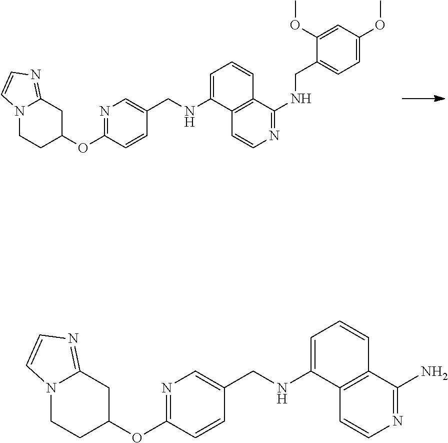 [SMILES: COc1ccc(CNc2nccc3c(NCc4ccc(OC5CCn6ccnc6C5)nc4)cccc23)c(OC)c1 ; Nc1nccc2c(NCc3ccc(OC4CCn5ccnc5C4)nc3)cccc12]

Deprotection of N1-[(2,4-dimethoxyphenyl)methyl]-N5-[[6-(5,6,7,8-tetrahydroimidazo[1,2-a]pyridin-7-yloxy)-3-pyridyl]methyl]isoquinoline-1,5-diamine (52 mg, 0.1 mmol) was carried out using General Method 12. The crude product was purified via automated prep HPLC (Mass directed 2-60% over 20 min in basic mobile phase) and lyophilised to afford the product (15 mg, 41% yield) as an off-white solid.

[M+H]⁺=387.2

¹H NMR (DMSO, 400 MHz) δ 2.18-2.26 (2H, m), 2.92 (1H, dd, J=16.8, 5.1 Hz), 3.15 (1H, dd, J=16.8, 4.6 Hz), 3.94-4.08 (2H, m), 4.39 (2H, d, J=5.8 Hz), 5.47-5.58 (1H, m), 6.49 (2H, s), 6.55 (1H, d, J=7.6 Hz), 6.66 (1H, t, J=6.0 Hz), 6.74 (1H, d, J=8.5 Hz), 6.82 (1H, d, J=1.2 Hz), 7.02 (1H, d, J=1.2 Hz), 7.12-7.19 (2H, m), 7.32 (1H, d, J=8.3 Hz), 7.70 (1H, dd, J=8.5, 2.5 Hz), 7.73 (1H, d, J=6.0 Hz), 8.21 (1H, d, J=2.4 Hz)

### Example Number 4319

### N5-[[2-[(2-methyl-5,6,7,8-tetrahydroimidazo[1,2-a]pyridin-7-yl)methoxy]-4-pyridyl]methyl]isoquinoline-1,5-diamine

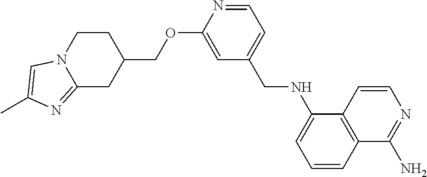 [SMILES: Cc1cn2c(n1)CC(COc1cc(CNc3cccc4c(N)nccc34)ccn1)CC2]

### 2-[(2-Methyl-5,6,7,8-tetrahydroimidazo[1,2-a]pyridin-7-yl)methoxy]pyridine-4-carbonitrile

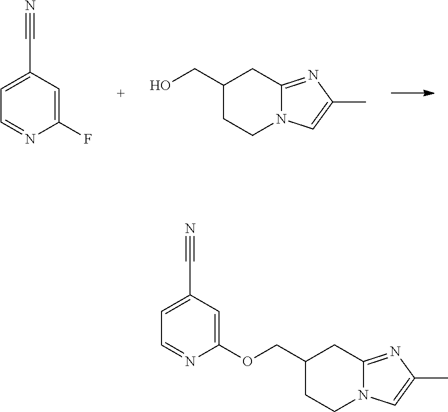 [SMILES: N#Cc1ccnc(F)c1 ; Cc1cn2c(n1)CC(CO)CC2 ; Cc1cn2c(n1)CC(COc1cc(C#N)ccn1)CC2]

Following General Method 1b, (2-methyl-5,6,7,8-tetrahydroimidazo[1,2-a]pyridin-7-yl)methanol (310 mg, 1.87 mmol) was reacted with 4-cyano-2-fluoropyridine (455 mg, 3.73 mmol) at 65° C. for 4 days. The reaction mixture was cooled to rt, filtered through filter paper and washed with EtOAc (50 mL). The filtrate was purified by flash chromatography (Silica, 0-100% EtOAc in Pet ether followed by 0-30% MeOH in EtOAc) to the product (285 mg, 55% yield) as a brown solid.

[M+H]⁺=269.1

¹H NMR (400 MHz, CDCl₃) δ 1.76-1.88 (m, 1H), 2.19 (s, 3H), 2.20-2.26 (m, 1H), 2.38-2.51 (m, 1H), 2.61 (dd, J=16.5, 10.7 Hz, 1H), 3.08 (ddd, J=16.5, 5.0, 1.5 Hz, 1H), 3.88 (td, J=11.7, 4.8 Hz, 1H), 4.03 (ddd, J=12.4, 5.7, 3.1 Hz, 1H), 4.28-4.42 (m, 2H), 6.52 (d, J=1.1 Hz, 1H), 7.01 (t, J=1.1 Hz, 1H), 7.09 (dd, J=5.2, 1.3 Hz, 1H), 8.28 (dd, J=5.2, 0.8 Hz, 1H)

### [2-[(2-Methyl-5,6,7,8-tetrahydroimidazo[1,2-a]pyridin-7-yl)methoxy]-4-pyridyl]methanamine

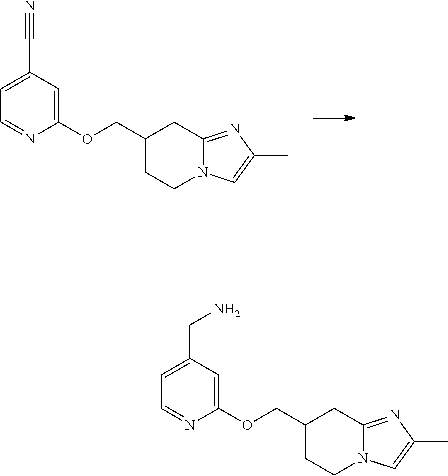 [SMILES: Cc1cn2c(n1)CC(COc1cc(C#N)ccn1)CC2 ; Cc1cn2c(n1)CC(COc1cc(CN)ccn1)CC2]

The nitrile, 2-[(2-methyl-5,6,7,8-tetrahydroimidazo[1,2-a]pyridin-7-yl)methoxy]pyridine-4-carbonitrile (285 mg, 1.06 mmol) was reduced according to General Method 3a, using Raney Ni for 1 h. The solvent was removed in vacuo to afford the product (270 mg, 86% yield) as a yellow oil.

[M+H]⁺=273.1

### N1-[(2,4-dimethoxyphenyl)methyl]-N5-[[2-[(2-methyl-5,6,7,8-tetrahydroimidazo[1,2-a]pyridin-7-yl)methoxy]-4-pyridyl]methyl]isoquinoline-1,5-diamine

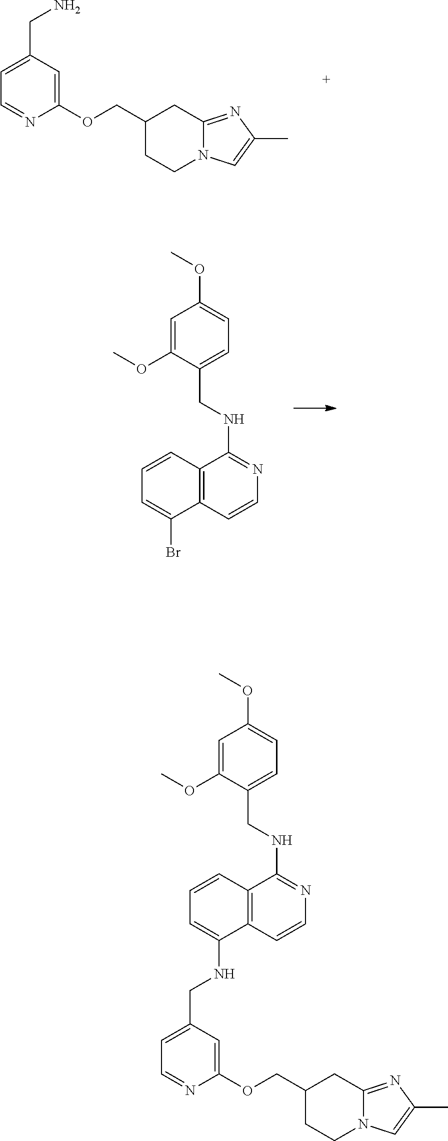

Using General Method 4, 5-bromo-N-[(2,4-dimethoxyphenyl)methyl]isoquinolin-1-amine (136 mg, 0.36 mmol), was reacted with [2-[(2-methyl-5,6,7,8-tetrahydroimidazo[1,2-a]pyridin-7-yl)methoxy]-4-pyridyl]methanamine (100 mg, 0.34 mmol) and NaOtBu (49 mg, 0.51 mmol) in 1,4-dioxane (5 mL) at 50° C. for 5 h. The reaction mixture was filtered through Celite®, washing with EtOAc (40 mL) and MeOH (10 mL) and concentrated. The crude product was purified by flash chromatography (Silica, 0-30% MeOH in DCM) to afford the product (134 mg, 66% yield) as an orange solid.

[M+H]1=565.3

### N5-[[2-[(2-methyl-5,6,7,8-tetrahydroimidazo[1,2-a]pyridin-7-yl)methoxy]-4-pyridyl]methyl]isoquinoline-1,5-diamine

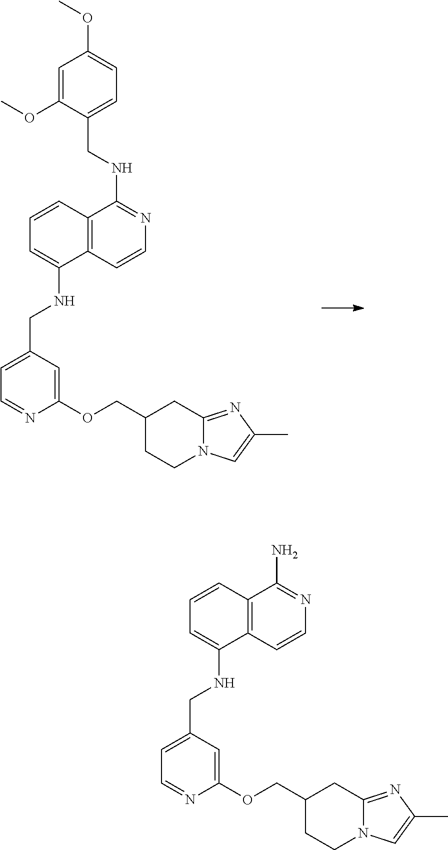

Deprotection of N1-[(2,4-dimethoxyphenyl)methyl]-N5-[[2-[(2-methyl-5,6,7,8-tetrahydroimidazo[1,2-a]pyridin-7-yl)methoxy]-4-pyridyl]methyl]isoquinoline-1,5-diamine (134 mg, 0.24 mmol) was carried out using General Method 12. The crude product was purified by flash chromatography (Silica, 22% MeOH in DCM) and the product was lyophilized to afford the product (31.0 mg, 38% yield) as an off white solid.

[M+H]⁺=415.2

### Example Number 9005

### N5-((3-Methyl-5-(3-(trifluoromethyl)-5,6-dihydro-[1,2,4]triazolo[4,3-a]pyrazin-7(8H)-yl)pyridin-2-yl)methyl)isoquinoline-1,5-diamine

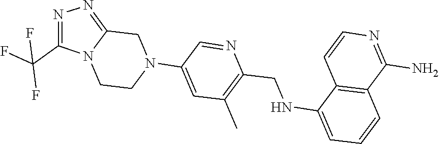 [SMILES: Cc1cc(N2CCn3c(nnc3C(F)(F)F)C2)cnc1CNc1cccc2c(N)nccc12]

### 3-Methyl-5-(3-(trifluoromethyl)-5,6-dihydro-[1,2,4]triazolo[4,3-a]pyrazin-7(8H)-yl)picolinonitrile

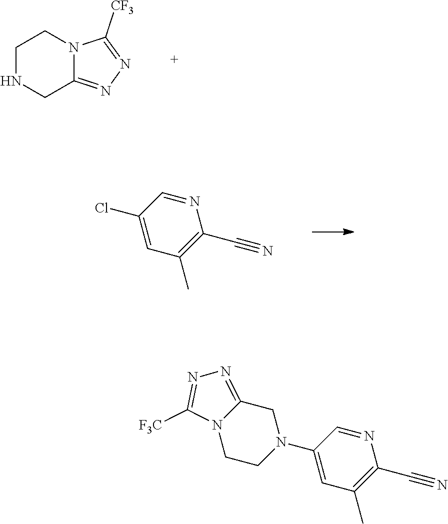

Following General Method 4 (using Ruphos Pd G3 as catalyst), 3-(trifluoromethyl)-5,6,7,8-tetrahydro-[1,2,4]triazolo[4,3-a]pyrazine (227 mg, 1.18 mmol) was reacted with 5-chloro-3-methylpicolinonitrile (150 mg, 983 μmol) in the presence of CsCO₃ (961 mg 2.95 mmol) and RuPhos (45.9 mg, 98.3 μmol) in 1,4-dioxane (3.5 mL) at 80° C. overnight. The crude product was purified by flash chromatography (Silica, 0-5% (0.7M NH₃ in MeOH) in DCM) to afford the product (172 mg, 57% yield) as a pale yellow solid.

[M+H]⁺=313.3

¹H NMR (DMSO, 500 MHz) δ 2.42 (s, 3H), 4.02 (t, J=5.4 Hz, 2H), 4.31 (t, J=5.4 Hz, 2H), 4.95 (s, 2H), 7.53 (d, J=2.9 Hz, 1H), 8.44 (d, J=2.9 Hz, 1H)

### (3-Methyl-5-(3-(trifluoromethyl)-5,6-dihydro-[1,2,4]triazolo[4,3-a]pyrazin-7(8H)-yl)pyridin-2-yl)methanamine

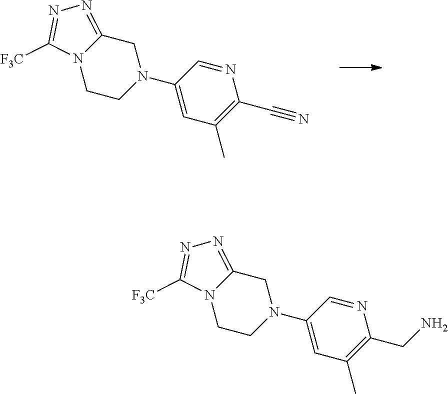 [SMILES: Cc1cc(N2CCn3c(nnc3C(F)(F)F)C2)cnc1C#N ; Cc1cc(N2CCn3c(nnc3C(F)(F)F)C2)cnc1CN]

3-Methyl-5-(3-(trifluoromethyl)-5,6-dihydro-[1,2,4]triazolo[4,3-a]pyrazin-7(8H)-yl)picolinonitrile (168 mg, 0.55 mmol) was reduced according to General Method 3a, using Raney Ni for 6 h. The solvent was removed in vacuo to afford the product (105 mg, 57% yield) as an off white solid.

[M+H]⁺=313.3

¹H NMR (DMSO, 500 MHz) δ 1.88 (2H, s), 2.26 (3H, s), 3.71 (2H, s), 3.80 (2H, t, J=5.5 Hz), 4.28 (2H, t, J=5.5 Hz), 4.70 (2H, s), 7.35 (1H, d, J=2.8 Hz), 8.21 (1H, d, J=2.8 Hz)

### N1-(2,4-Dimethoxybenzyl)-N5-((3-methyl-5-(3-(trifluoromethyl)-5,6-dihydro-[1,2,4]triazolo[4,3-a]pyrazin-7(8H)-yl)pyridin-2-yl)methyl)isoquinoline-1,5-diamine

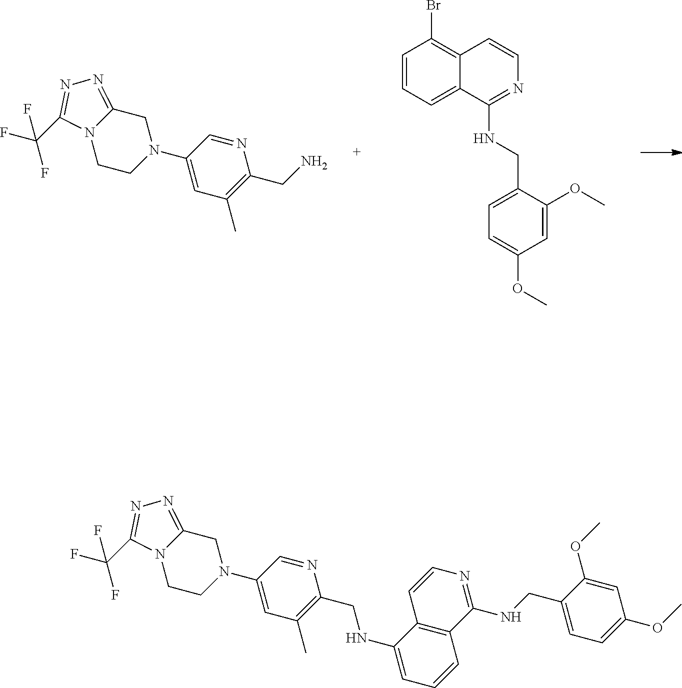 [SMILES: Cc1cc(N2CCn3c(nnc3C(F)(F)F)C2)cnc1CN ; COc1ccc(CNc2nccc3c(Br)cccc23)c(OC)c1 ; COc1ccc(CNc2nccc3c(NCc4ncc(N5CCn6c(nnc6C(F)(F)F)C5)cc4C)cccc23)c(OC)c1]

(3-Methyl-5-(3-(trifluoromethyl)-5,6-dihydro-[1,2,4]triazolo[4,3-a]pyrazin-7(8H)-yl)pyridin-2-yl)methanamine (102 mg, 304 μmol) and 5-bromo-N-(2,4-dimethoxybenzyl)isoquinolin-1-amine (113 mg, 304 μmol) were reacted according to General Method 4 using Brettphos Pd G4 (14.0 mg, 0.05 Eq, 0.015 mmol) and CsCO₃ (198 mg, 0.61 mmol) in 1,4-dioxane (2 mL). The mixture was diluted with EtOAc and concentrated onto silica. Flash chromatography (Silica, 0-5% (0.7M NH₃ in MeOH) in DCM) afforded the product (60 mg, 31% yield) as a beige solid.

[M+H]⁺=605.5

¹H NMR (DMSO, 500 MHz) δ 2.38 (3H, s), 3.71 (3H, s), 3.78-3.87 (5H, m), 4.29 (2H, t, J=5.5 Hz), 4.41 (2H, d, J=4.6 Hz), 4.59 (2H, d, J=5.6 Hz), 4.74 (2H, s), 6.38 (1H, dd, J=8.4, 2.4 Hz), 6.51-6.61 (2H, m), 6.79 (1H, d, J=7.8 Hz), 6.97-7.06 (2H, m), 7.25 (1H, t, J=8.0 Hz), 7.37-7.51 (3H, m), 7.77 (1H, d, J=6.1 Hz), 8.30 (1H, d, J=2.8 Hz)

### N5-((3-Methyl-5-(3-(trifluoromethyl)-5,6-dihydro-[1,2,4]triazolo[4,3-a]pyrazin-7(8H)-yl)pyridin-2-yl)methyl)isoquinoline-1,5-diamine

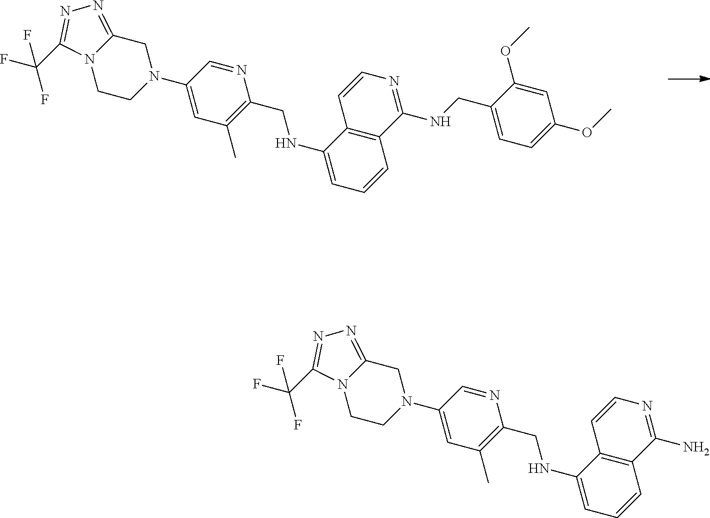 [SMILES: COc1ccc(CNc2nccc3c(NCc4ncc(N5CCn6c(nnc6C(F)(F)F)C5)cc4C)cccc23)c(OC)c1 ; Cc1cc(N2CCn3c(nnc3C(F)(F)F)C2)cnc1CNc1cccc2c(N)nccc12]

Deprotection of N1-(2,4-dimethoxybenzyl)-N5-((3-methyl-5-(3-(trifluoromethyl)-5,6-dihydro-[1,2,4]triazolo[4,3-a]pyrazin-7(8H)-yl)pyridin-2-yl)methyl)isoquinoline-1,5-diamine (57 mg, 0.09 mmol) was carried out using General Method 12. Flash chromatography (Silica, 0-10% (0.7 M NH₃ in MeOH) in DCM) afforded the product (33 mg, 80% yield) as a white solid.

[M+H]⁺=455.4

¹H NMR (DMSO, 500 MHz) δ 2.37 (3H, s), 3.84 (2H, t, J=5.5 Hz), 4.29 (2H, t, J=5.4 Hz), 4.40 (2H, d, J=4.6 Hz), 4.74 (2H, s), 6.45-6.60 (3H, m), 6.77 (1H, d, J=7.7 Hz), 7.05 (1H, d, J=6.1 Hz), 7.21 (1H, t, J=8.0 Hz), 7.34 (1H, d, J=8.3 Hz), 7.43 (1H, d, J=2.7 Hz), 7.76 (1H, d, J=6.1 Hz), 8.30 (1H, d, J=2.8 Hz)

### Example Number 1282

### N5-((4-Methyl-6-(3-(trifluoromethyl)-5,6-dihydro-[1,2,4]triazolo[4,3-a]pyrazin-7(8H)-yl)pyridin-3-yl)methyl)isoquinoline-1,5-diamine

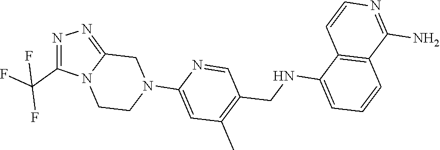 [SMILES: Cc1cc(N2CCn3c(nnc3C(F)(F)F)C2)ncc1CNc1cccc2c(N)nccc12]

### N5-((6-Chloro-4-methylpyridin-3-yl)methyl)-N1-(2,4-dimethoxybenzyl)isoquinoline-1,5-diamine

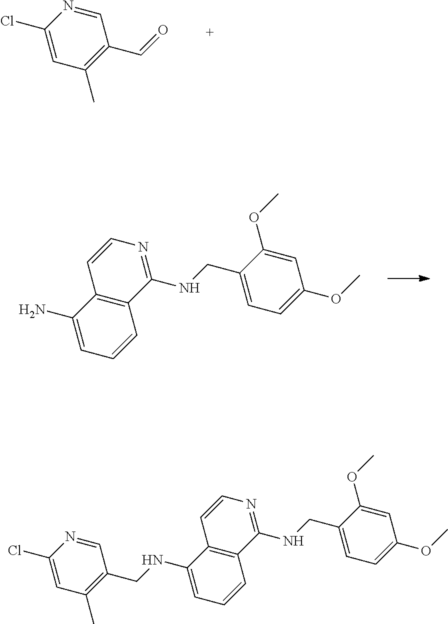

A mixture of 6-chloro-4-methylnicotinaldehyde (468 mg, 3.01 mmol) and N1-(2,4-dimethoxybenzyl)isoquinoline-1,5-diamine (621 mg, 2.01 mmol) in dichloroethane (25 mL) was treated with acetic acid (241 mg, 4.01 mmol) and the mixture stirred at 65° C. for 22 h then at rt for 96 h. Additional material from a previous reaction was added and the combined mixture partitioned between DCM (50 mL) and sat. NaHCO₃ (aq) (50 mL) and the organic layer collected. The aqueous layer was washed with further DCM (50 mL) and the combined organics concentrated under vacuum. The residue was suspended in MeOH (21 mL) and heated to 60° C. before the slow portion-wise addition of NaBH₄ (1.49 g, 39.4 mmol). After completion of the addition and stirring for 20 min, further NaBH₄ (759 mg, 20.1 mmol) was added portion-wise. THF (10 mL) was added and the mixture treated with further NaBH₄ (759 mg, 20.1 mmol) portion-wise. After 15 min solvents were removed under vacuum and the residue partitioned between DCM (50 mL) and sat. NaHCO₃ (aq) (50 mL). The aqueous layer was washed with further DCM (50 mL) and the combined organics washed with brine (50 mL), dried (Na₂SO₄), filtered and concentrated under vacuum. Flash chromatography (Silica, 0-3% (0.7M NH₃ in MeOH) in DCM) followed by further flash chromatography (Silica, 0-70% EtOAc/Iso-Hexanes) afforded the product (980 mg, 51% yield) as a white foam. Mixed fractions were combined and re-purified by flash chromatography (Silica, 0-70% EtOAc/Iso-Hexanes) to afford further product (156 mg, 9% yield).

[M+H]⁺=449.4/451.4

¹H NMR (DMSO, 500 MHz) δ 2.40 (3H, d, J=0.7 Hz), 3.71 (3H, s), 3.82 (3H, s), 4.44 (2H, d, J=5.4 Hz), 4.59 (2H, d, J=5.6 Hz), 6.39 (1H, dd, J=8.3, 2.4 Hz), 6.52 (1H, d, J=7.8 Hz), 6.55 (1H, d, J=2.4 Hz), 6.57 (1H, t, J=5.6 Hz), 7.02 (1H, d, J=8.4 Hz), 7.17 (1H, d, J=6.1 Hz), 7.21 (1H, t, J=8.0 Hz), 7.39 (1H, s), 7.42 (1H, t, J=6.0 Hz), 7.49 (1H, d, J=8.4 Hz), 7.75 (1H, d, J=6.1 Hz), 8.14 (1H, s)

### N1-(2,4-Dimethoxybenzyl)-N5-((4-methyl-6-(3-(trifluoromethyl)-5,6-dihydro-[1,2,4]triazolo[4,3-a]pyrazin-7(8H)-yl)pyridin-3-yl)methyl)isoquinoline-1,5-diamine

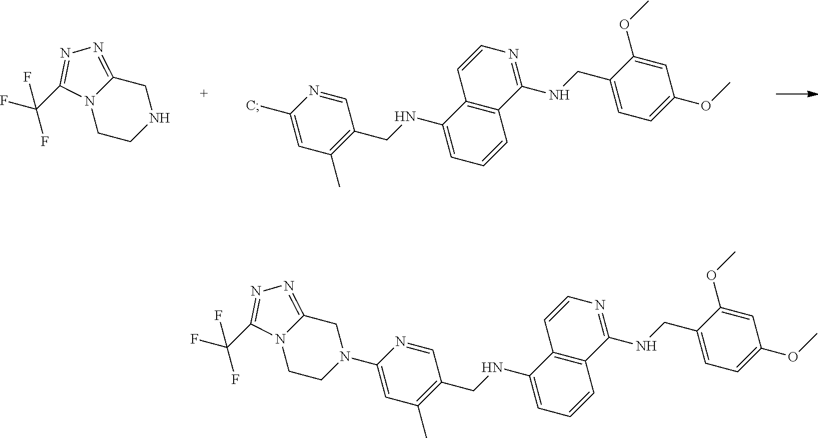

Following General Method 4 (using Ruphos Pd G3 as catalyst), 3-(trifluoromethyl)-5,6,7,8-tetrahydro-[1,2,4]triazolo[4,3-a]pyrazine (358 mg, 1.86 mmol) was reacted with N5-((6-chloro-4-methylpyridin-3-yl)methyl)-N1-(2,4-dimethoxybenzyl)isoquinoline-1,5-diamine (750 mg, 1.55 mmol) in the presence of CsCO₃ (1.52 g, 4.66 mmol) and RuPhos (72.5 mg, 0.1 Eq, 155 μmol) in 1,4-dioxane (12 mL) at 80° C. for 20 h. The crude product was purified by flash chromatography (Silica, 0-4% (0.7M NH₃ in MeOH) in DCM) to afford the product (692 mg, 69% yield) as a brown solid.

[M+H]⁺=605.2

¹H NMR (DMSO, 500 MHz) δ 2.35 (3H, s), 3.71 (3H, s), 3.82 (3H, s), 4.07 (2H, t, J=5.5 Hz), 4.22 (2H, t, J=5.4 Hz), 4.33 (2H, d, J=5.1 Hz), 4.59 (2H, d, J=5.6 Hz), 4.94 (2H, s), 6.35-6.42 (2H, m), 6.54 (1H, d, J=2.4 Hz), 6.58 (1H, d, J=7.8 Hz), 6.98 (1H, s), 7.01 (1H, d, J=8.4 Hz), 7.15 (1H, d, J=6.2 Hz), 7.21 (1H, t, J=8.0 Hz), 7.39 (1H, t, J=5.9 Hz), 7.45 (1H, d, J=8.4 Hz), 7.72 (1H, d, J=6.1 Hz), 8.00 (1H, s)

### N5-((4-Methyl-6-(3-(trifluoromethyl)-5,6-dihydro-[1,2,4]triazolo[4,3-a]pyrazin-7(8H)-yl)pyridin-3-yl)methyl)isoquinoline-1,5-diamine

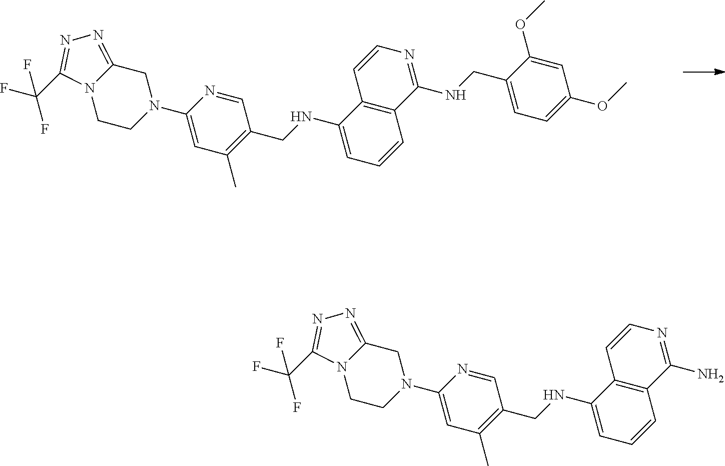 [SMILES: COc1ccc(CNc2nccc3c(NCc4cnc(N5CCn6c(nnc6C(F)(F)F)C5)cc4C)cccc23)c(OC)c1 ; Cc1cc(N2CCn3c(nnc3C(F)(F)F)C2)ncc1CNc1cccc2c(N)nccc12]

Deprotection of N1-(2,4-dimethoxybenzyl)-N5-((4-methyl-6-(3-(trifluoromethyl)-5,6-dihydro-[1,2,4]triazolo[4,3-a]pyrazin-7(8H)-yl)pyridin-3-yl)methyl)isoquinoline-1,5-diamine (689 mg, 1.07 mmol) was carried out using General Method 12. The crude product was purified by automated prep HPLC (mass directed 30-60% over 16 min in basic mobile phase) then lyophilized to afford the product (315 mg, 65% yield) as a white solid.

[M+H]⁺=455.2

¹H NMR (DMSO-d6, 500 MHz) δ 2.34 (3H, s), 4.07 (2H, t, J=5.4 Hz), 4.22 (2H, t, J=5.4 Hz), 4.32 (2H, d, J=5.3 Hz), 4.94 (2H, s), 6.33 (1H, t, J=5.4 Hz), 6.49 (2H, s), 6.56 (1H, d, J=7.7 Hz), 6.98 (1H, s), 7.14-7.20 (2H, m), 7.33 (1H, d, J=8.3 Hz), 7.72 (1H, d, J=6.1 Hz), 8.01 (1H, s)

### Example Numbers 1303, 1304 and 1305

### N5-((4-Methyl-6-(8-methyl-3-(trifluoromethyl)-5,6-dihydro-[1,2,4]triazolo[4,3-a]pyrazin-7(8H)-yl)pyridin-3-yl)methyl)isoquinoline-1,5-diamine

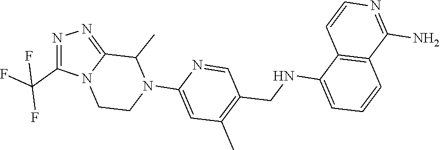 [SMILES: Cc1cc(N2CCn3c(nnc3C(F)(F)F)C2C)ncc1CNc1cccc2c(N)nccc12]

### N1-(2,4-Dimethoxybenzyl)-N5-((4-methyl-6-(8-methyl-3-(trifluoromethyl)-5,6-dihydro-[1,2,4]triazolo[4,3-a]pyrazin-7(8H)-yl)pyridin-3-yl)methyl)isoquinoline-1,5-diamine

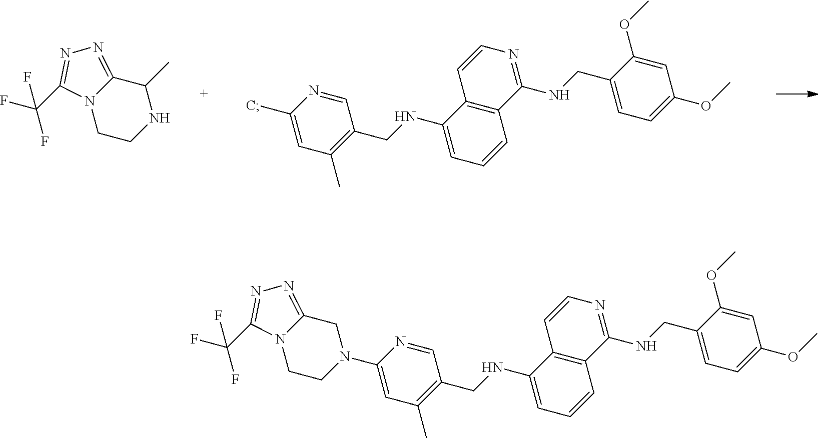

Following General Method 4 (using Ruphos Pd G3 as catalyst), 8-methyl-3-(trifluoromethyl)-5,6,7,8-tetrahydro-[1,2,4]triazolo[4,3-a]pyrazine (109 mg, 527 μmol) was reacted with N5-((6-chloro-4-methylpyridin-3-yl)methyl)-N1-(2,4-dimethoxybenzyl)isoquinoline-1,5-diamine (212 mg, 439 μmol), in the presence of CsCO₃ (429 mg, 1.32 mmol) and RuPhos (20.5 mg, 43.9 μmol) in 1,4-dioxane (3.4 mL) at 80° C. for 18 h. The crude product was purified by flash chromatography (Silica, 0-4% (0.7M NH₃ in MeOH) in DCM) to afford the product (201 mg, 69% yield) as a brown solid.

[M+H]⁺=619.2

¹H NMR (DMSO, 500 MHz) δ 1.51 (3H, d, J=6.8 Hz), 2.34 (3H, s), 3.51 (1H, ddd, J=14.9, 11.6, 3.9 Hz), 3.71 (3H, s), 3.82 (3H, s), 4.08 (1H, td, J=12.0, 4.4 Hz), 4.23 (1H, dd, J=12.0, 3.6 Hz), 4.32 (2H, d, J=5.1 Hz), 4.59 (2H, d, J=5.6 Hz), 4.68 (1H, dd, J=14.6, 4.3 Hz), 5.89 (1H, q, J=6.8 Hz), 6.30-6.41 (2H, m), 6.54 (1H, d, J=2.4 Hz), 6.59 (1H, d, J=7.8 Hz), 6.93 (1H, s), 7.01 (1H, d, J=8.4 Hz), 7.16 (1H, d, J=6.1 Hz), 7.21 (1H, t, J=8.0 Hz), 7.39 (1H, t, J=5.9 Hz), 7.45 (1H, d, J=8.4 Hz), 7.72 (1H, d, J=6.1 Hz), 7.99 (1H, s)

### N5-((4-Methyl-6-(8-methyl-3-(trifluoromethyl)-5,6-dihydro-[1,2,4]triazolo[4,3-a]pyrazin-7(8H)-yl)pyridin-3-yl)methyl)isoquinoline-1,5-diamine

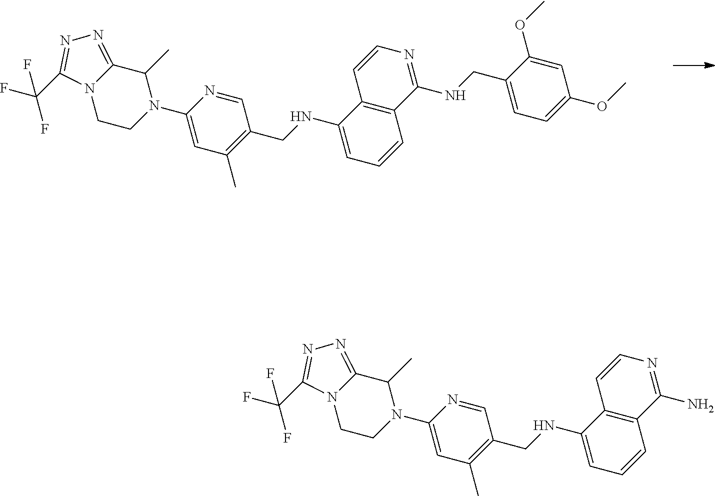 [SMILES: COc1ccc(CNc2nccc3c(NCc4cnc(N5CCn6c(nnc6C(F)(F)F)C5C)cc4C)cccc23)c(OC)c1 ; Cc1cc(N2CCn3c(nnc3C(F)(F)F)C2C)ncc1CNc1cccc2c(N)nccc12]

Deprotection of N1-(2,4-dimethoxybenzyl)-N5-((4-methyl-6-(8-methyl-3-(trifluoromethyl)-5,6-dihydro-[1,2,4]triazolo[4,3-a]pyrazin-7(8H)-yl)pyridin-3-yl)methyl)isoquinoline-1,5-diamine was carried out using General Method 12. The crude product was purified by flash chromatography (Silica, 0-8% (0.7 M NH₃ in MeOH) in DCM) to afford the racemic product (Example Number 1303) (135 mg, 93% yield) as a beige solid.

¹H NMR (DMSO, 500 MHz) δ 1.51 (3H, d, J=6.8 Hz), 2.34 (3H, s), 3.46-3.56 (1H, m), 4.06-4.12 (1H, m), 4.20-4.26 (1H, m), 4.31 (2H, d, J=5.1 Hz), 4.68 (1H, dd, J=14.5, 4.3 Hz), 5.89 (1H, q, J=6.8 Hz), 6.31 (1H, t, J=5.4 Hz), 6.48 (2H, s), 6.57 (1H, d, J=7.7 Hz), 6.93 (1H, s), 7.14-7.20 (2H, m), 7.33 (1H, d, J=8.3 Hz), 7.71 (1H, d, J=6.1 Hz), 7.99 (1H, s)

### (R*)-N5-((4-Methyl-6-(8-methyl-3-(trifluoromethyl)-5,6-dihydro-[1,2,4]triazolo[4,3-a]pyrazin-7(8H)-yl)pyridin-3-yl)methyl)isoquinoline-1,5-diamine and (S*)-N5-((4-Methyl-6-(8-methyl-3-(trifluoromethyl)-5,6-dihydro-[1,2,4]triazolo[4,3-a]pyrazin-7(8H)-yl)pyridin-3-yl)methyl)isoquinoline-1,5-diamine

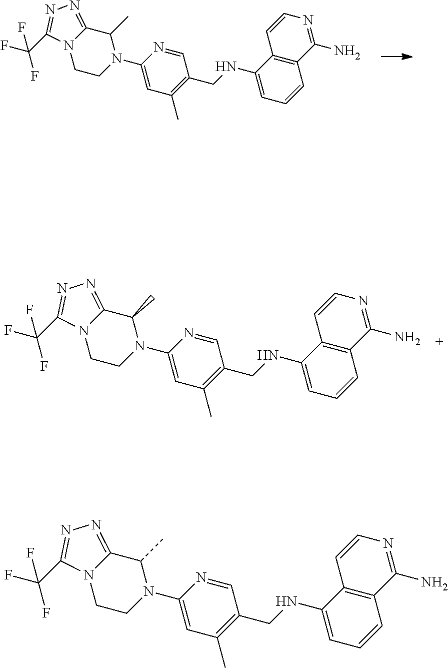 [SMILES: Cc1cc(N2CCn3c(nnc3C(F)(F)F)C2C)ncc1CNc1cccc2c(N)nccc12 ; Cc1cc(N2CCn3c(nnc3C(F)(F)F)[C@H]2C)ncc1CNc1cccc2c(N)nccc12 ; Cc1cc(N2CCn3c(nnc3C(F)(F)F)[C@@H]2C)ncc1CNc1cccc2c(N)nccc12]

The enantiomers were separated by chiral SFC on a Sepiatec with UV detection by DAD at 220 nm, 40° C., 120 bar. The column was IG 10×250 mm, 5 μm, flow rate 20 mL/min at 40% MeOH, 60% CO₂ to afford the first eluting isomer (R*)-N5-((4-methyl-6-(8-methyl-3-(trifluoromethyl)-5,6-dihydro-[1,2,4]triazolo[4,3-a]pyrazin-7(8H)-yl)pyridin-3-yl)methyl)isoquinoline-1,5-diamine (Example Number 1304, stereochemistry not confirmed) (50.9 mg, 36% yield)

[M+H]+=469.2

and the second eluting isomer (S*)-N5-((4-methyl-6-(8-methyl-3-(trifluoromethyl)-5,6-dihydro-[1,2,4]triazolo[4,3-a]pyrazin-7(8H)-yl)pyridin-3-yl)methyl)isoquinoline-1,5-diamine (Example Number 1305, stereochemistry not confirmed) (55.1 mg, 39% yield)

[M+H]+=469.2

¹H NMR (DMSO-d6, 500 MHz) δ 1.51 (3H, d, J=6.8 Hz), 2.34 (3H, s), 3.51 (1H, ddd, J=15.0, 11.6, 3.8 Hz), 4.08 (1H, td, J=11.9, 4.4 Hz), 4.23 (1H, dd, J=12.4, 3.6 Hz), 4.31 (2H, d, J=5.3 Hz), 4.68 (1H, dd, J=14.6, 4.3 Hz), 5.89 (1H, q, J=6.8 Hz), 6.31 (1H, t, J=5.5 Hz), 6.49 (2H, s), 6.57 (1H, d, J=7.7 Hz), 6.93 (1H, s), 7.13-7.21 (2H, m), 7.33 (1H, d, J=8.3 Hz), 7.71 (1H, d, J=6.1 Hz), 7.99 (1H, s)

### Example Numbers 1314, 1315 and 1316

### 2-Chloro-N-((4-methyl-6-(8-methyl-3-(trifluoromethyl)-5,6-dihydro-[1,2,4]triazolo[4,3-a]pyrazin-7(8H)-yl)pyridin-3-yl)methyl)-1H-pyrrolo[2,3-b]pyridin-4-amine

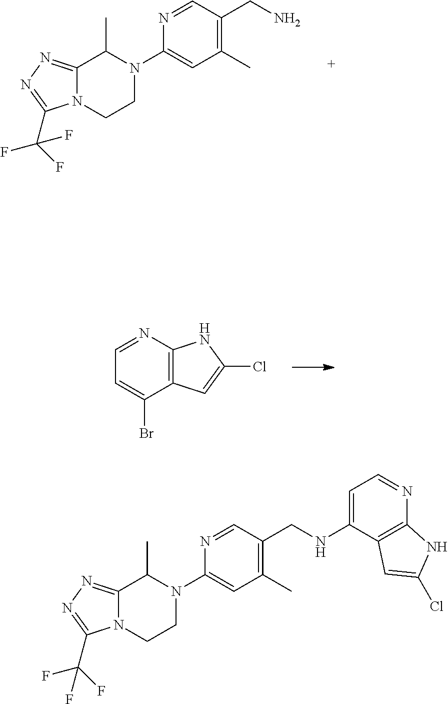 [SMILES: Cc1cc(N2CCn3c(nnc3C(F)(F)F)C2C)ncc1CN ; Clc1cc2c(Br)ccnc2[nH]1 ; Cc1cc(N2CCn3c(nnc3C(F)(F)F)C2C)ncc1CNc1ccnc2[nH]c(Cl)cc12]

To a mixture of (4-methyl-6-(8-methyl-3-(trifluoromethyl)-5,6-dihydro-[1,2,4]triazolo[4,3-a]pyrazin-7(8H)-yl)pyridin-3-yl)methanamine (97.1 mg, 262 umol), 4-bromo-2-chloro-1H-pyrrolo[2,3-b]pyridine (77.0 mg, 249 μmol) and BrettPhos Pd G3 (11.3 mg, 12.5 μmol) in degassed 1,4-dioxane (1.3 mL) was added a solution of lithium bis(trimethylsilyl)amide (1M in THF) (599 μL 599 μmol). The mixture was purged with N₂ (g) and heated at 70° C. for 1 h. Additional lithium bis(trimethylsilyl)amide (1M in THF) (299 μL, 299 μmol) was added and mixture heated at 70° C. for a further 1 h. Further BrettPhos Pd G3 (11.3 mg, 12.5 μmol) and 1,4-dioxane (1.0 mL) were added and the mixture heated for a further 1 h. On cooling, AcOH (0.4 mL) and MeOH (10 mL) were added to form a solution. The crude solution was loaded onto SCX and washed with MeOH. The product was eluted with 0.7M NH₃ in MeOH and the eluent concentrated. Flash chromatography (Silica, 0-9% (0.7M NH₃ in MeOH) in DCM) afforded the racemic product (Example number 1314) (43.5 mg, 35%) as a light yellow solid.

1H NMR (DMSO, 500 MHz) δ 1.52 (3H, d, J=6.8 Hz), 2.30 (3H, s), 3.52 (1H, ddd, J=15.0, 11.6, 3.8 Hz), 4.08(1H, td, J=12.0, 4.4 Hz), 4.24 (1H, dd, J=12.3, 3.6 Hz), 4.32 (2H, d, J=5.3 Hz), 4.70 (1H, dd, J=14.5, 4.3 Hz), 5.90 (1H, q, J=6.8 Hz), 6.19 (1H, d, J=5.7 Hz), 6.59 (1H, s), 6.82 (1H, t, J=5.4 Hz), 6.95 (1H, s), 7.76 (1H, d, J=5.6 Hz), 8.02 (1H, s), 11.97 (1H, s)

### (R*)-2-Chloro-N-((4-methyl-6-(8-methyl-3-(trifluoromethyl)-5,6-dihydro-[1,2,4]triazolo[4,3-a]pyrazin-7(8H)-yl)pyridin-3-yl)methyl)-1H-pyrrolo[2,3-b]pyridin-4-amine and (S*)-2-Chloro-N-((4-methyl-6-(8-methyl-3-(trifluoromethyl)-5,6-dihydro-[1,2,4]triazolo[4,3-a]pyrazin-7(8H)-yl)pyridin-3-yl)methyl)-1H-pyrrolo[2,3-b]pyridin-4-amine

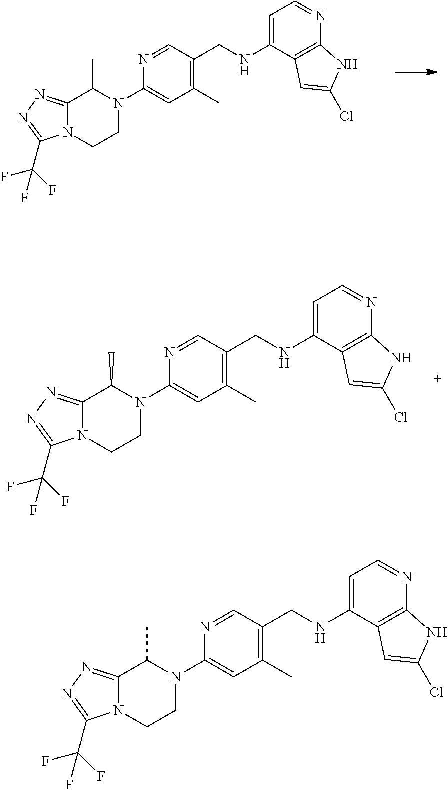 [SMILES: Cc1cc(N2CCn3c(nnc3C(F)(F)F)C2C)ncc1CNc1ccnc2[nH]c(Cl)cc12 ; Cc1cc(N2CCn3c(nnc3C(F)(F)F)[C@H]2C)ncc1CNc1ccnc2[nH]c(Cl)cc12 ; Cc1cc(N2CCn3c(nnc3C(F)(F)F)[C@@H]2C)ncc1CNc1ccnc2[nH]c(Cl)cc12]

The enantiomers were separated by chiral SFC on a Waters prep 100 with PDA and QDA detectors, 40° C., 120 bar. The column was a Chiralpak A1, 5 μM, 21 mm×250 mm; flow rate 65 mL/min of 45% MeOH (neutral), 55% CO₂ to afford the first eluting isomer (11.9 mg, 9.4%) and the second eluting isomer (11.8 mg, 9.2%) identified as Example Numbers 1315 and 1316 (stereochemistries not confirmed).

### Example Number 1278

### N-((6-(3-(Difluoromethyl)-5,6-dihydro-[1,2,4]triazolo[4,3-a]pyrazin-7(8H)-yl)pyridin-3-yl)methyl)-2-methyl-1H-pyrrolo[2,3-b]pyridin-4-amine

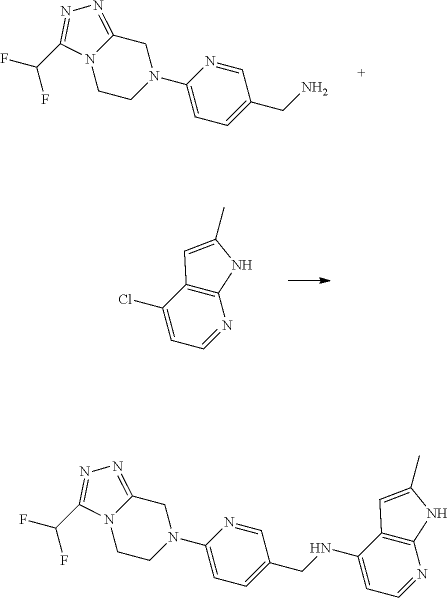 [SMILES: NCc1ccc(N2CCn3c(nnc3C(F)F)C2)nc1 ; Cc1cc2c(Cl)ccnc2[nH]1 ; Cc1cc2c(NCc3ccc(N4CCn5c(nnc5C(F)F)C4)nc3)ccnc2[nH]1]

To a mixture of (6-(3-(difluoromethyl)-5,6-dihydro-[1,2,4]triazolo[4,3-a]pyrazin-7(8H)-yl)pyridin-3-yl)methanamine (88.3 mg, 315 μmol), 4-chloro-2-methyl-1H-pyrrolo[2,3-b]pyridine (50.0 mg, 300 μmol) and BrettPhos Pd G3 (13.6 mg, 15.0 μmol) was added a solution of lithium bis(trimethylsilyl)amide (1M in THF) (720 μL, 720 μmol). The mixture purged with N₂ (g) and heated at 70° C. for 2 h. On cooling, AcOH (0.2 mL) and MeOH (1 mL) were added. This was stirred for 5 min then diluted with MeOH (15 mL). The solution was loaded onto SCX and washed with MeOH. The product was eluted with 0.7M NH₃ in MeOH. Flash chromatography (Silica, 0-8% (0.7M NH₃ in MeOH) in DCM) afforded the product (23.7 mg, 19% yield) as a pale yellow solid.

[M+H]⁺=411.3

¹H NMR (500 MHz, DMSO-d6) 2.30 (3H, s), 4.05 (2H, t, J=5.5 Hz), 4.19 (2H, t, J=5.5 Hz), 4.34 (2H, d, J=6.0 Hz), 4.90 (2H, s), 6.07 (1H, d, J=5.6 Hz), 6.22 (1H, s), 6.87 (1H, t, J=6.2 Hz), 7.05 (1H, d, J=8.7 Hz), 7.35 (1H, t, J=51.9 Hz), 7.62 (1H, dd, J=8.7, 2.4 Hz), 7.65 (1H, d, J=5.5 Hz), 8.20 (1H, d, J=2.3 Hz), 10.97 (1H, s)

### Example Numbers 10002, 10003 and 10004

### 5-(2-(6-(3-(Trifluoromethyl)-5,6-dihydro-[1,2,4]triazolo[4,3-a]pyrazin-7(8H)-yl)pyridin-3-yl)pyrrolidin-1-yl)isoquinolin-1-amine

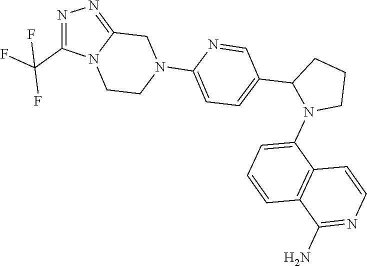 [SMILES: Nc1nccc2c(N3CCCC3c3ccc(N4CCn5c(nnc5C(F)(F)F)C4)nc3)cccc12]

### 7-(5-(Pyrrolidin-2-yl)pyridin-2-yl)-3-(trifluoromethyl)-5,6,7,8-tetrahydro-[1,2,4]triazolo[4,3-a]pyrazine

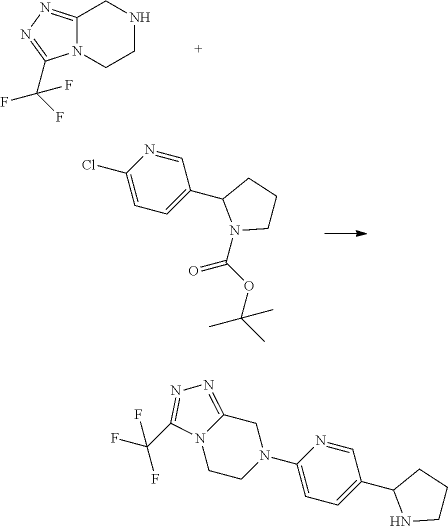 [SMILES: FC(F)(F)c1nnc2n1CCNC2 ; CC(C)(C)OC(=O)N1CCCC1c1ccc(Cl)nc1 ; FC(F)(F)c1nnc2n1CCN(c1ccc(C3CCCN3)cn1)C2]

Following General Method 4 (using Ruphos Pd G3 as catalyst), tert-butyl 2-(6-chloropyridin-3-yl)pyrrolidine-1-carboxylate (600 mg, 2.12 mmol) was reacted with 3-(trifluoromethyl)-5,6,7,8-tetrahydro-[1,2,4]triazolo[4,3-a]pyrazine (408 mg, 2.12 mmol) in the presence of NaOtBu (408 mg, 4.24 mmol) in 1,4-dioxane (10 mL) at 90° C. for 2 h. On cooling, AcOH (2 mL) was added along with MeOH (10 mL) and the crude product loaded onto SCX with MeOH and washed with MeOH. The product was eluted with 0.7M NH₃ in MeOH. The product was redissolved in a mixture of DCM (10.5 mL) and TFA (3.5 mL) and stirred at rt for 2 h. The crude product was loaded onto SCX with MeCN and washed with MeOH. The product was eluted with 0.7M NH₃ in MeOH. Flash chromatography (Silica, 0-10% (0.7M NH₃ in MeOH) in DCM) afforded the product (513 mg, 69% yield) as a pale yellow solid.

[M+H]⁺=339.4

¹H NMR (DMSO, 500 MHz) δ 1.37-1.50 (1H, m), 1.64-1.82 (2H, m), 1.98-2.09 (1H, m), 2.63 (1H, brs), 2.78-2.87 (1H, m), 2.93-3.03 (1H, m), 3.92 (1H, t, J=7.6 Hz), 4.08 (2H, t, J=5.5 Hz), 4.24 (2H, t, J=5.4 Hz), 4.95 (2H, s), 7.05 (1H, d, J=8.7 Hz), 7.62 (1H, dd, J=8.7, 2.4 Hz), 8.13 (1H, d, J=2.4 Hz)

### N-(2,4-dimethoxybenzyl)-5-(2-(6-(3-(trifluoromethyl)-5,6-dihydro-[1,2,4]triazolo[4,3-a]pyrazin-7(8H)-yl)pyridin-3-yl)pyrrolidin-1-yl)isoquinolin-1-amine

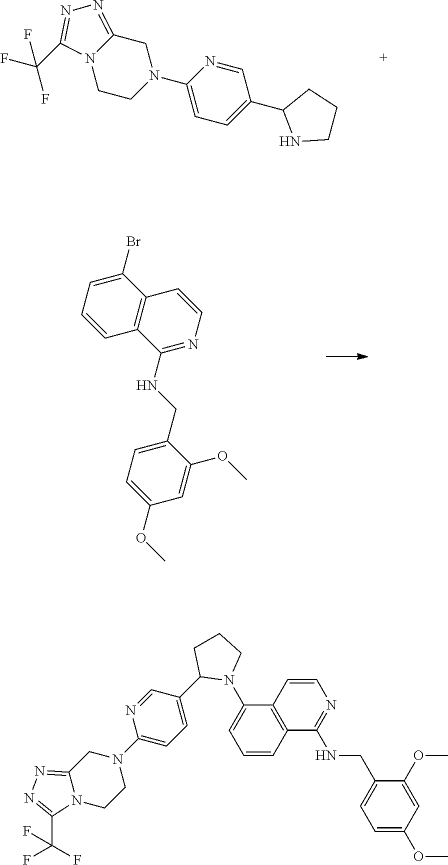 [SMILES: FC(F)(F)c1nnc2n1CCN(c1ccc(C3CCCN3)cn1)C2 ; COc1ccc(CNc2nccc3c(Br)cccc23)c(OC)c1 ; COc1ccc(CNc2nccc3c(N4CCCC4c4ccc(N5CCn6c(nnc6C(F)(F)F)C5)nc4)cccc23)c(OC)c1]

7-(5-(Pyrrolidin-2-yl)pyridin-2-yl)-3-(trifluoromethyl)-5,6,7,8-tetrahydro-[1,2,4]triazolo[4,3-a]pyrazine (326 mg, 965 μmol), 5-bromo-N-(2,4-dimethoxybenzyl)isoquinolin-1-amine (300 mg, 804 μmol), CsCO₃ (550 mg, 1.69 mmol), (9,9-dimethyl-9H-xanthene-4,5-diyl)bis(diphenylphosphane) (163 mg, 281 μmol) and Pd₂(dba)₃ (95.7 mg, 104 μmol) were combined in a flask and the flask evacuated and purged with N₂ (g). Anhydrous 1,4-dioxane (7.5 mL) was added and the mixture evacuated and purged with N₂ (g) The mixture was heated to 100° C. for 18 h. Additional (9,9-dimethyl-9H-xanthene-4,5-diyl)bis(diphenylphosphane) (93.0 mg, 161 μmol) and Pd₂(dba)₃ (73.6 mg, 80.4 μmol) were added and the mixture evacuated and purged with N₂ (g) and heated to 100° C. for 24 h. On cooling, the mixture was treated with AcOH (1 mL) and sonicated. MeOH (20 mL) was added and the crude product loaded onto SCX and washed with MeOH. The product was eluted with 0.7M NH₃ in MeOH. Flash chromatography (Silica, 0-6% (0.7M NH₃ in MeOH) in DCM) afforded the product (85 mg, 15% yield) as a yellow solid.

[M+H]⁺=631.6

¹H NMR (DMSO, 500 MHz) δ 1.79-1.97 (2H, m), 2.05-2.14 (1H, m), 2.33-2.41 (1H, m), 2.88-2.95 (1H, m), 3.70 (3H, s), 3.81 (3H, s), 3.97-4.08 (3H, m), 4.18 (2H, t, J=5.4 Hz), 4.54 (1H, dd, J=15.8, 5.6 Hz), 4.61 (1H, dd, J=15.8, 5.6 Hz), 4.68-4.74 (1H, m), 4.86 (2H, d, J=3.3 Hz), 6.38 (1H, dd, J=8.4, 2.4 Hz), 6.54 (1H, d, J=2.4 Hz), 6.92 (1H, d, J=8.8 Hz), 7.01 (1H, d, J=8.4 Hz), 7.12 (1H, d, J=7.8 Hz), 7.17-7.26 (2H, m), 7.51 (1H, t, J=5.9 Hz), 7.62 (1H, dd, J=8.8, 2.4 Hz), 7.74-7.83 (2H, m), 8.19 (1H, d, J=2.3 Hz)

### 5-(2-(6-(3-(Trifluoromethyl)-5,6-dihydro-[1,2,4]triazolo[4,3-a]pyrazin-7(8H)-yl)pyridin-3-yl)pyrrolidin-1-yl)isoquinolin-1-amine

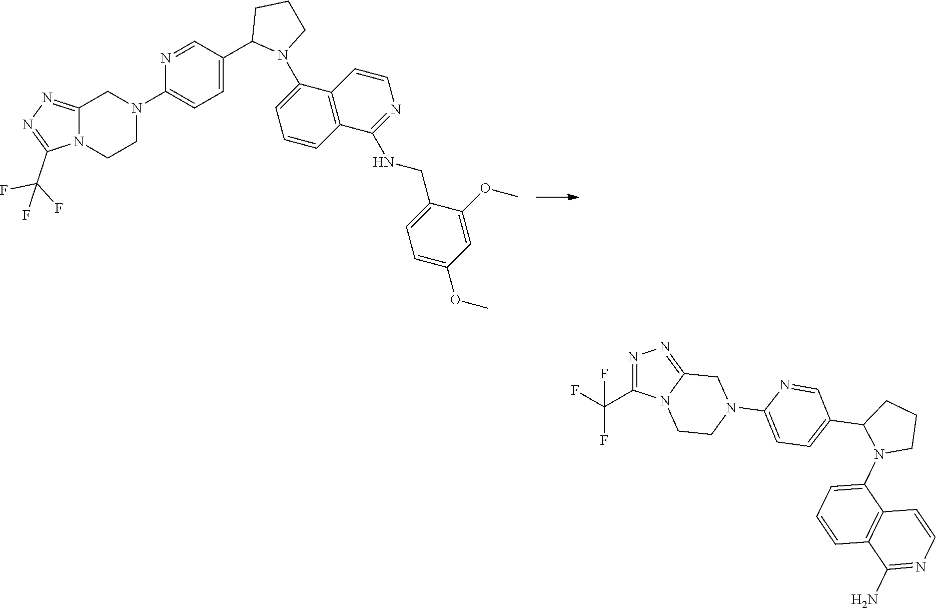 [SMILES: COc1ccc(CNc2nccc3c(N4CCCC4c4ccc(N5CCn6c(nnc6C(F)(F)F)C5)nc4)cccc23)c(OC)c1 ; Nc1nccc2c(N3CCCC3c3ccc(N4CCn5c(nnc5C(F)(F)F)C4)nc3)cccc12]

Deprotection of N-(2,4-dimethoxybenzyl)-5-(2-(6-(3-(trifluoromethyl)-5,6-dihydro-[1,2,4]triazolo[4,3-a]pyrazin-7(8H)-yl)pyridin-3-yl)pyrrolidin-1-yl)isoquinolin-1-amine (78 mg, 90% Wt, 1 Eq, 0.11 mmol) was carried out according to General Method 12. Flash chromatography (Silica, 0-10% (0.7M NH₃ in MeOH) in DCM) afforded the racemic product (Example Number 10002) (50.6 mg, 90% yield) as a pale yellow solid.

[M+H]⁺=481.2

¹H NMR (DMSO, 500 MHz) δ 1.75-1.97 (2H, m), 2.04-2.14 (1H, m), 2.32-2.41 (1H, m), 2.86-2.95 (1H, m), 3.91-4.08 (3H, m), 4.12-4.22 (2H, m), 4.70 (1H, t, J=7.9 Hz), 4.80-4.91 (2H, m), 6.59 (2H, s), 6.92 (1H, d, J=8.8 Hz), 7.09 (1H, d, J=7.6 Hz), 7.15-7.26 (2H, m), 7.61 (1H, d, J=8.7 Hz), 7.66 (1H, d, J=8.2 Hz), 7.78 (1H, dd, J=6.0, 1.7 Hz), 8.18 (1H, s)

### (S*)-5-(2-(6-(3-(Trifluoromethyl)-5,6-dihydro-[1,2,4]triazolo[4,3-a]pyrazin-7(8H)-yl)pyridin-3-yl)pyrrolidin-1-yl)isoquinolin-1-amine and (R*)-5-(2-(6-(3-(Trifluoromethyl)-5,6-dihydro-[1,2,4]triazolo[4,3-a]pyrazin-7(8H)-yl)pyridin-3-yl)pyrrolidin-1-yl)isoquinolin-1-amine

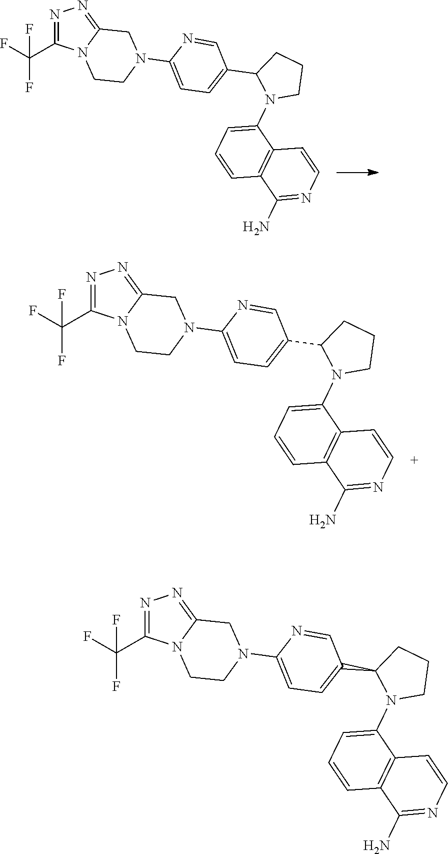

The enantiomers were separated by chiral HPLC on a Gilson UV directed prep with UV detection at 222 nm, 25° C. The column was a iC5 20×250 mm, 5 um, flow rate 20 mL/min at 25% Water (0.1% DEA), 75% MeCN to afford the first eluting isomer (S*)-5-(2-(6-(3-(trifluoromethyl)-5,6-dihydro-[1,2,4]triazolo[4,3-a]pyrazin-7(8H)-yl)pyridin-3-yl)pyrrolidin-1-yl)isoquinolin-1-amine (Example Number 10003, stereochemistry not confirmed) (18.4 mg, 33%).

[M+H]⁺=481.2

and the second eluting isomer (R*)-5-(2-(6-(3-(trifluoromethyl)-5,6-dihydro-[1,2,4]triazolo[4,3-a]pyrazin-7(8H)-yl)pyridin-3-yl)pyrrolidin-1-yl)isoquinolin-1-amine (Example Number 10004, stereochemistry not confirmed) (14.5 mg, 27%).

[M+H]⁺=481.2

### Example Number 8459

### N5-((4-(3-(Trifluoromethyl)-5,6-dihydro-[1,2,4]triazolo[4,3-a]pyrazin-7(8H)-yl)thiophen-2-yl)methyl)isoquinoline-1,5-diamine

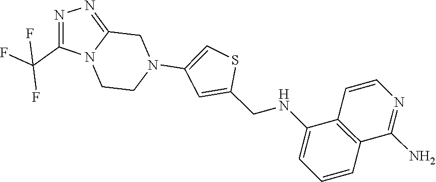 [SMILES: Nc1nccc2c(NCc3cc(N4CCn5c(nnc5C(F)(F)F)C4)cs3)cccc12]

### N5-((4-Bromothiophen-2-yl)methyl)-N1-(2,4-dimethoxybenzyl)isoquinoline-1,5-diamine

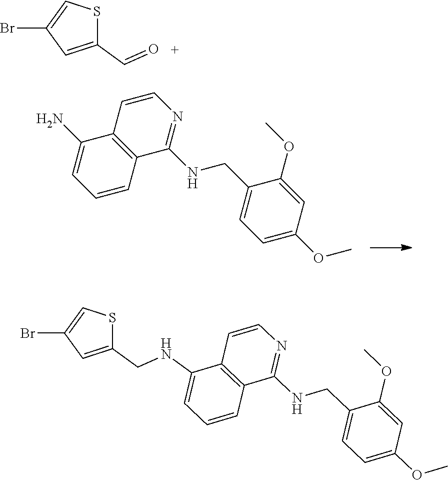

A mixture of 4-bromothiophene-2-carbaldehyde (0.19 g, 0.97 mmol) and N1-(2,4-dimethoxybenzyl)isoquinoline-1,5-diamine (0.30 g, 0.97 mmol) in dichloroethane (15 mL) was treated with AcOH (0.12 g, 1.9 mmol) and the mixture stirred at 65° C. for 18 h. The mixture was partitioned between DCM (50 mL) and sat. NaHCO₃ (aq) (50 mL) and the organic layer collected. The aqueous layer was washed with further DCM (50 mL) and the combined organics concentrated in vacuo. The residue was suspended in MeOH (10 mL) and THF (5 mL) heated to 60° C. before the slow portion-wise addition of NaBH₄ (0.37 g, 9.7 mmol). After 15 min sat. NaHCO₃ (aq) (20 mL) and DCM (20 mL) were added. The aqueous layer was washed with further DCM (50 mL) and the combined organics washed with brine (50 mL), dried (Na₂SO₄), filtered and concentrated in vacuo. Flash chromatography (Silica, 0-70% EtOAc/Iso-Hexanes) afforded the product (0.40 g, 72% yield) as a clear brown oil.

[M+H]⁺

¹H NMR (DMSO, 500 MHz) δ 3.71 (3H, s), 3.82 (3H, s), 4.61 (4H, dd, J=19.1, 5.7 Hz), 6.39 (1H, dd, J=8.4, 2.4 Hz), 6.55 (1H, d, J=2.4 Hz), 6.62-6.67 (1H, m), 6.84 (1H, t, J=6.0 Hz), 7.02 (1H, d, J=8.4 Hz), 7.07-7.13 (2H, m), 7.22 (1H, t, J=8.0 Hz), 7.40-7.47 (2H, m), 7.49 (1H, d, J=8.4 Hz), 7.75 (1H, d, J=6.1 Hz)

### N1-(2,4-Dimethoxybenzyl)-N5-((4-(3-(trifluoromethyl)-5,6-dihydro-[1,2,4]triazolo[4,3-a]pyrazin-7(8H)-yl)thiophen-2-yl)methyl)isoquinoline-1,5-diamine

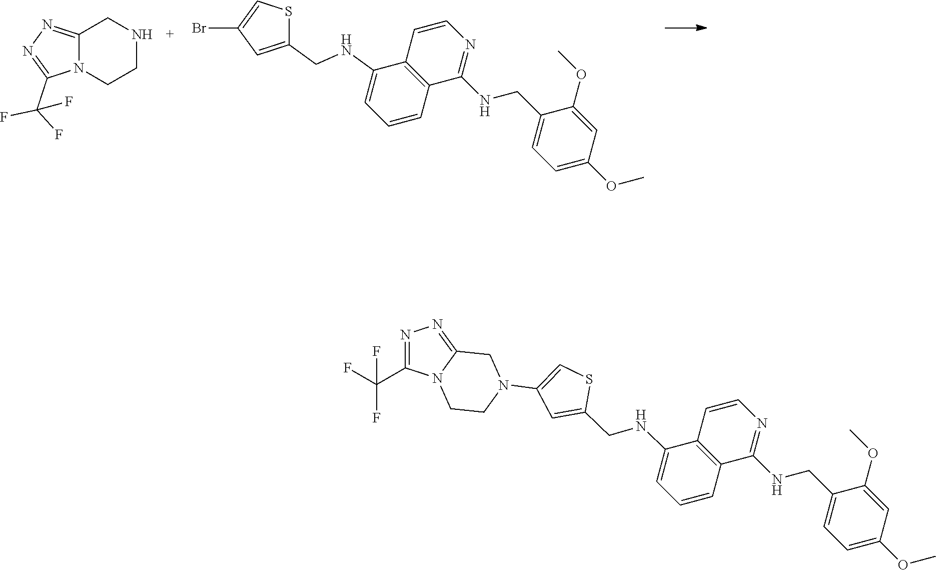 [SMILES: FC(F)(F)c1nnc2n1CCNC2 ; COc1ccc(CNc2nccc3c(NCc4cc(Br)cs4)cccc23)c(OC)c1 ; COc1ccc(CNc2nccc3c(NCc4cc(N5CCn6c(nnc6C(F)(F)F)C5)cs4)cccc23)c(OC)c1]

Following General Method 4 (using Ruphos Pd G3 as catalyst), N5-((4-bromothiophen-2-yl)methyl)-N1-(2,4-dimethoxybenzyl)isoquinoline-1,5-diamine (400 mg, 826 μmol) was reacted with 3-(trifluoromethyl)-5,6,7,8-tetrahydro-[1,2,4]triazolo[4,3-a]pyrazine (190 mg, 991 μmol) in the presence of CsCO₃ (807 mg, 2.48 mmol) and RuPhos (38.5 mg, 82.6 μmol) in 1,4-dioxane (5 mL) at 80° C. 18 h. The crude product was purified via flash chromatography (Silica, 0-20% (0.7 M NH₃ in MeOH) in DCM) to afford the product (300 mg, 48% yield) as a clear brown oil.

[M+H]⁺=596.0

¹H NMR (CDCl₃, 500 MHz) δ 1.01 (1H, dt, J=13.4, 6.6 Hz), 1.08-1.20 (2H, m), 3.51 (1H, t, J=5.5 Hz), 3.78 (3H, s), 3.83 (3H, s), 4.14 (1H, t, J=5.5 Hz), 4.45 (1H, s), 4.53-4.63 (2H, m), 4.72 (2H, d, J=5.3 Hz), 4.83 (1H, t, J=5.5 Hz), 5.72 (1H, d, J=6.0 Hz), 6.19 (1H, d, J=1.8 Hz), 6.42 (1H, dt, J=8.2, 1.9 Hz), 6.48 (1H, d, J=2.4 Hz), 6.60-6.79 (1H, m), 6.79-6.88 (2H, m), 7.01-7.12 (1H, m), 7.17-7.28 (1H, m), 7.28 (1H, d, J=8.3 Hz), 7.99 (1H, dd, J=6.1, 3.0 Hz)

### N5-((4-(3-(Trifluoromethyl)-5,6-dihydro-[1,2,4]triazolo[4,3-a]pyrazin-7(8H)-yl)thiophen-2-yl)methyl)isoquinoline-1,5-diamine

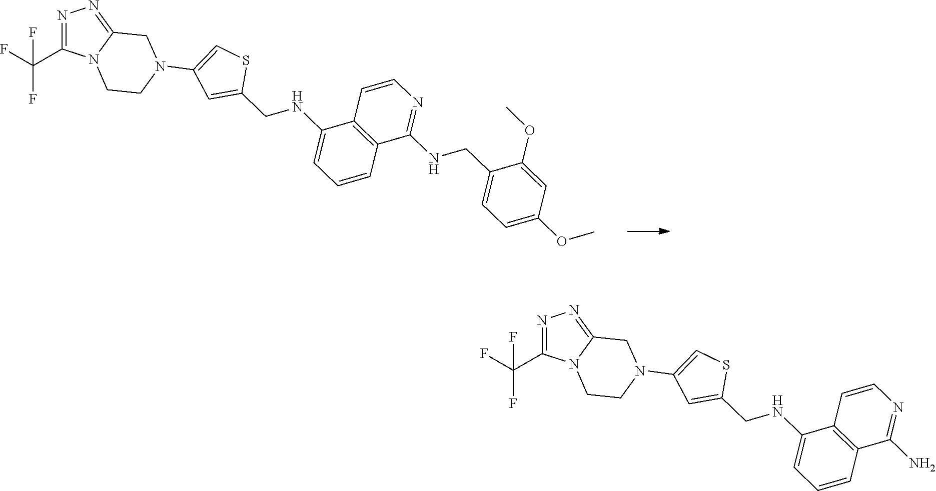 [SMILES: COc1ccc(CNc2nccc3c(NCc4cc(N5CCn6c(nnc6C(F)(F)F)C5)cs4)cccc23)c(OC)c1 ; Nc1nccc2c(NCc3cc(N4CCn5c(nnc5C(F)(F)F)C4)cs3)cccc12]

Deprotection of N1-(2,4-dimethoxybenzyl)-N5-((4-(3-(trifluoromethyl)-5,6-dihydro-[1,2,4]triazolo[4,3-a]pyrazin-7(8H)-yl)thiophen-2-yl)methyl)isoquinoline-1,5-diamine (350 mg, 588 μmol) was carried out using General Method 12. The crude product was purified via automated prep HPLC (mass directed 20-100% over 12.5 min in basic mobile phase) to afford the product (160 mg, 61% yield) as a pale yellow solid

[M+H]⁺=445.9

¹H NMR (DMSO, 500 MHz) δ 3.63 (2H, t, J=5.5 Hz), 4.24 (2H, t, J=5.5 Hz), 4.50-4.57 (4H, m), 6.44 (1H, d, J=1.8 Hz), 6.50 (2H, s), 6.62 (1H, d, J=7.7 Hz), 6.73 (1H, t, J=5.9 Hz), 7.11-7.16 (2H, m), 7.16 (1H, t, J=8.0 Hz), 7.35 (1H, d, J=8.3 Hz), 7.74 (1H, d, J=6.0 Hz)

### Example Number 1313

### 2-Chloro-N-((4-methyl-6-(3-(trifluoromethyl)-5,6-dihydro-[1,2,4]triazolo[4,3-a]pyrazin-7(8H)-yl)pyridin-3-yl)methyl)-1H-pyrrolo[2,3-b]pyridin-4-amine

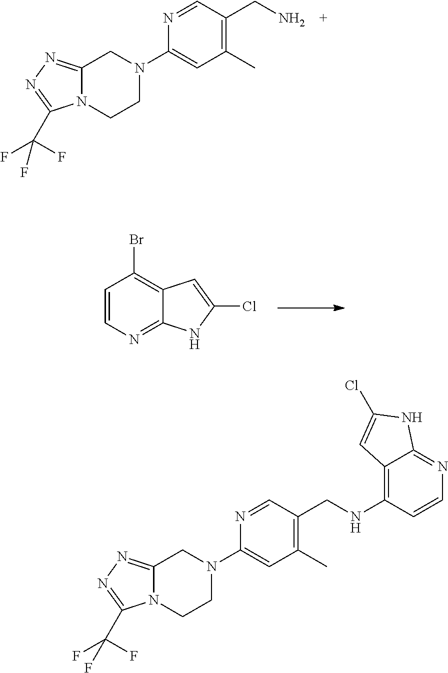 [SMILES: Cc1cc(N2CCn3c(nnc3C(F)(F)F)C2)ncc1CN ; Clc1cc2c(Br)ccnc2[nH]1 ; Cc1cc(N2CCn3c(nnc3C(F)(F)F)C2)ncc1CNc1ccnc2[nH]c(Cl)cc12]

To a mixture of (4-methyl-6-(3-(trifluoromethyl)-5,6-dihydro-[1,2,4]triazolo[4,3-a]pyrazin-7(8H)-yl)pyridin-3-yl)methanamine (85.7 mg, 242 μmol), 4-bromo-2-chloro-1H-pyrrolo[2,3-b]pyridine (71.0 mg, 230 μmol) and BrettPhos Pd G3 (10.4 mg, 11.5 μmol) in degassed 1,4-dioxane (1.2 mL) was added lithium bis(trimethylsilyl)amide (1M in THF) (552 μL, 552 μmol). The mixture was purged with N₂ (g) and heated at 70° C. for 1 h. On cooling, AcOH (0.4 mL) and MeOH (10 mL) were added to form a solution. The solution was loaded onto SCX and washed with MeOH. The product was eluted with 0.7M NH₃ in MeOH and the eluent concentrated. Flash chromatography (Silica, 0-8% (0.7M NH₃ in MeOH) in DCM) afforded the product (54 mg, 50%) as an off-white solid.

[M+H]⁺=463.3

¹H NMR (DMSO, 500 MHz) δ 2.31 (3H, s), 4.08 (2H, t, J=5.4 Hz), 4.23 (2H, t, J=5.4 Hz), 4.33 (2H, d, J=5.3 Hz), 4.96 (2H, s), 6.18 (1H, d, J=5.7 Hz), 6.58 (1H, s), 6.84 (1H, t, J=5.4 Hz), 6.99 (1H, s), 7.76 (1H, d, J=5.6 Hz), 8.04 (1H, s), 11.98 (1H, s)

### Example Number 1311

### N5-((6-(6-Methyl-3-(trifluoromethyl)-5,6-dihydro-[1,2,4]triazolo[4,3-a]pyrazin-7(8H)-yl)pyridin-3-yl)methyl)isoquinoline-1,5-diamine

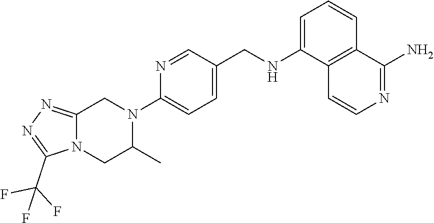 [SMILES: CC1Cn2c(nnc2C(F)(F)F)CN1c1ccc(CNc2cccc3c(N)nccc23)cn1]

### 6-Methyl-3-(trifluoromethyl)-5,6,7,8-tetrahydro-[1,2,4]triazolo[4,3-a]pyrazine

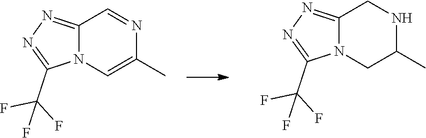

A solution of 6-methyl-3-(trifluoromethyl)-[1,2,4]triazolo[4,3-a]pyrazine (223 mg, 1.10 mmol) and Pd/C (117 mg, 110 μmol) in MeOH (8 mL) were placed in a hydrogenator vessel, purged with N₂ (g) followed by H₂ (g) then stirred at rt under 2.5 bar of H₂ (g) for 6.5 h. The mixture was filtered, combined with a previous batch, and concentrated in vacuo, to afford the product as a pale yellow solid (77% overall yield).

[M+H]⁺=207.2

¹H NMR (CDCl3, 500 MHz) δ 1.37 (3H, d, J=6.4 Hz), 3.29-3.39 (1H, m), 3.71 (1H, t, J=11.4 Hz), 4.17 (1H, dd, J=12.3, 4.1 Hz), 4.25 (1H, d, J=16.6 Hz), 4.53 (1H, d, J=16.5 Hz) [NH proton not observed]

### N1-(2,4-Dimethoxybenzyl)-N5-((6-(6-methyl-3-(trifluoromethyl)-5,6-dihydro-[1,2,4]triazolo[4,3-a]pyrazin-7(8H)-yl)pyridin-3-yl)methyl)isoquinoline-1,5-diamine

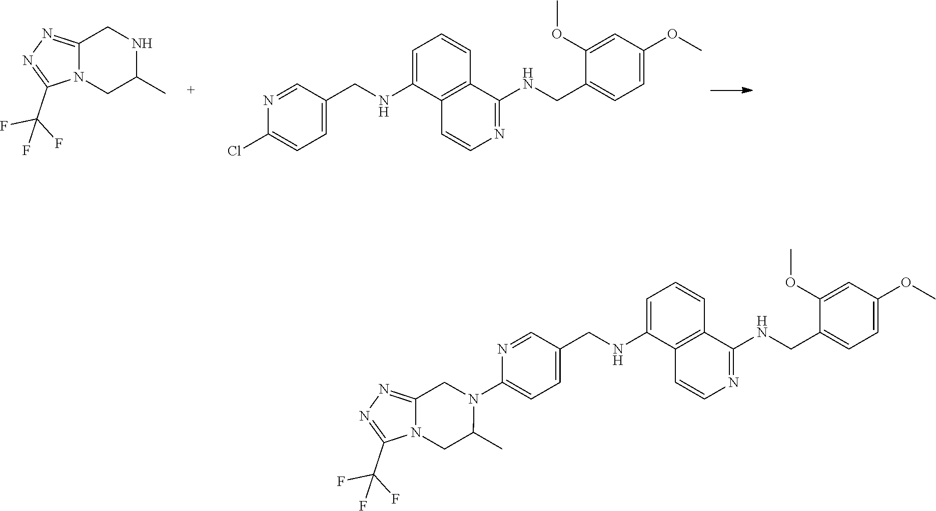 [SMILES: CC1Cn2c(nnc2C(F)(F)F)CN1 ; COc1ccc(CNc2nccc3c(NCc4ccc(Cl)nc4)cccc23)c(OC)c1 ; COc1ccc(CNc2nccc3c(NCc4ccc(N5Cc6nnc(C(F)(F)F)n6CC5C)nc4)cccc23)c(OC)c1]

Following General Method 4 (using Ruphos Pd G3 as catalyst), 6-methyl-3-(trifluoromethyl)-5,6,7,8-tetrahydro-[1,2,4]triazolo[4,3-a]pyrazine (97.0 mg, 470 μmol) was reacted with N5-((6-chloropyridin-3-yl)methyl)-N1-(2,4-dimethoxybenzyl)isoquinoline-1,5-diamine (200 mg, 428 μmol), in the presence of RuPhos (20.0 mg, 42.8 μmol) and CsCO₃ (418 mg, 1.28 mmol) in 1,4-dioxane (4 mL) at 80° C. for 17 h. The reaction mixture was cooled to rt, combined with a previous batch, and diluted with EtOAc. The resulting solution was filtered over Celite® and concentrated in vacuo. The residue was purified by flash chromatography (Silica, 24 g cartridge, eluted with 0-20% (0.7M NH in MeOH) in DCM), to afford the product as a brown oil. This was dissolved in 10 mL MeOH, 0.15 mL AcOH was added, and the mixture was passed through an SCX cartridge, washed with 10 mL MeOH, and eluted with 3M NH₃ in MeOH (50 mL). The ammoniacal fraction was concentrated in vacuo, to afford the product as a brown solid (64% overall yield).

[M+H]⁺=605.0

### N5-((6-(6-Methyl-3-(trifluoromethyl)-5,6-dihydro-[1,2,4]triazolo[4,3-a]pyrazin-7(8H)-yl)pyridin-3-yl)methyl)isoquinoline-1,5-diamine

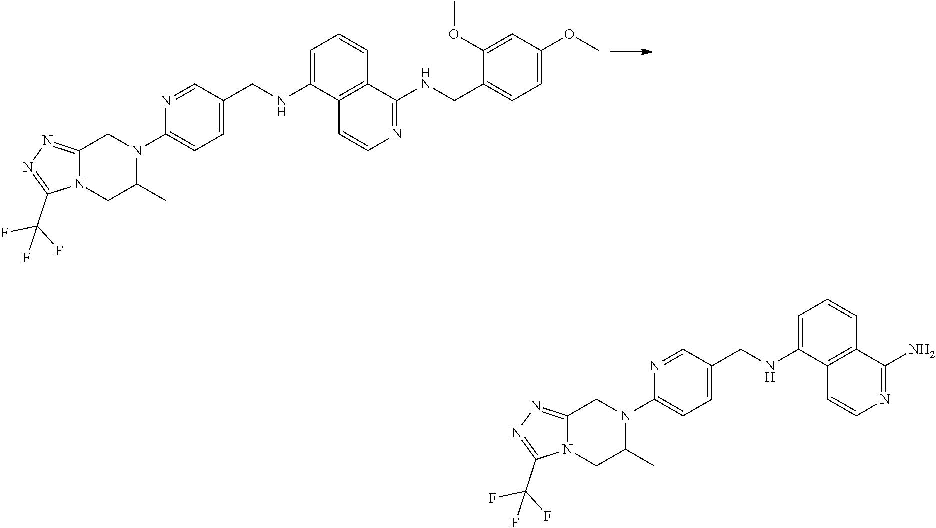 [SMILES: COc1ccc(CNc2nccc3c(NCc4ccc(N5Cc6nnc(C(F)(F)F)n6CC5C)nc4)cccc23)c(OC)c1 ; CC1Cn2c(nnc2C(F)(F)F)CN1c1ccc(CNc2cccc3c(N)nccc23)cn1]

Deprotection of N1-(2,4-dimethoxybenzyl)-N5-((6-(6-methyl-3-(trifluoromethyl)-5,6-dihydro-[1,2,4]triazolo[4,3-a]pyrazin-7(8H)-yl)pyridin-3-yl)methyl)isoquinoline-1,5-diamine (249 mg, 412 μmol) was carried out according to General Method 12. The reaction mixture was concentrated in vacuo, diluted with MeOH (5 mL), and passed through an SCX cartridge, washing with further MeOH (15 mL). The product was eluted with a solution of 3M NH in MeOH (30 mL). Flash chromatography (silica, 12 g cartridge, eluted with 0-20% (0.7M NH inMeOH) in DCM) afforded the product (93 mg, 49% yield) as an orange solid.

[M+H]⁺=455.4

¹H NMR (DMSO, 500 MHz) δ 1.05 (3H, d, J=6.8 Hz), 4.21 (1H, d, J=12.6 Hz), 4.29-4.34 (1H, m), 4.36 (2H, d, J=5.9 Hz), 4.43 (1H, d, J=17.3 Hz), 5.20-5.27 (2H, m), 6.48 (2H, s), 6.56 (1H, d, J=7.7 Hz), 6.63 (1H, t, J=6.0 Hz), 6.97 (1H, d, J=8.7 Hz), 7.14 (1H, t, J=8.0 Hz), 7.17 (1H, d, J=6.1 Hz), 7.31 (1H, d, J=8.3 Hz), 7.66 (1H, dd, J=8.7, 2.4 Hz), 7.73 (1H, d, J=6.1 Hz), 8.26 (1H, d, J=2.4 Hz).

### Example Number 1251

### 2-Chloro-N-((6-(((1-methylpiperidin-4-yl)methyl)amino)pyridin-3-yl)methyl)-1H-pyrrolo[2,3-c]pyridin-4-amine

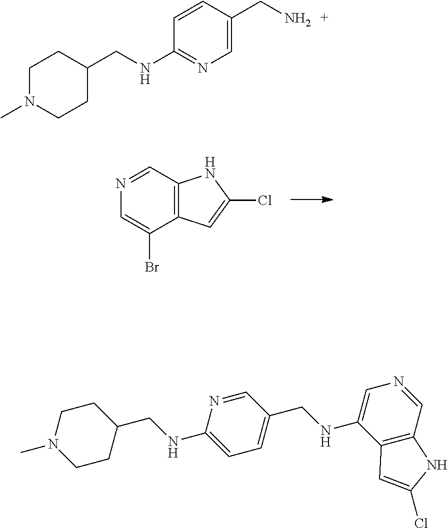 [SMILES: CN1CCC(CNc2ccc(CN)cn2)CC1 ; Clc1cc2c(Br)cncc2[nH]1 ; CN1CCC(CNc2ccc(CNc3cncc4[nH]c(Cl)cc34)cn2)CC1]

To a mixture of 5-(aminomethyl)-N-((1-methylpiperidin-4-yl)methyl)pyridin-2-amine (93.0 mg, 383 μmol) and 4-bromo-2-chloro-1H-pyrrolo[2,3-c]pyridine (108 mg, 421 μmol) in THF (2 mL) was added BrettPhos-Pd-G3 (17.4 mg, 19.1 μmol). The mixture was degassed with N₂ (g) then lithium bis(trimethylsilyl)amide (1M in THF) (919 μL, 919 μmol) was added drop-wise. The mixture was heated at 70° C. for 3 days. The mixture was concentrated in vacuo. The residue was resuspended in 1,4-dioxane (2 mL), then treated with tBuBrettPhos Pd G3 (16.4 mg, 19.1 μmol). The mixture was degassed with N₂ (g), then lithium bis(trimethylsilyl)amide (1M in THF) (919 μL, 919 μmol) was added drop-wise. The mixture was heated at 80° C. for 1.5 h under N₂ (g) The mixture was cooled to rt and treated with AcOH (0.2 mL). It was loaded onto SCX resin and eluted with MeOH followed by 7 N NH₃/MeOH. The crude product was purified by automated preparative HPLC (mass directed, 0.3% ammonia in water-MeCN, 10-100% MeCN gradient over 18.5 min) to obtain the product (14.5 mg, 9.7% yield) as a pale brown solid.

[M+H]⁺=385.3

¹H NMR (500 MHz, Methanol-d₄) δ 1.25-1.39 (m, 2H), 1.56-1.68 (m, 1H), 1.79-1.86 (m, 2H), 1.98-2.06 (m, 2H), 2.28 (s, 3H), 2.85-2.96 (m, 2H), 3.18 (d, J=6.9 Hz, 2H), 4.35 (s, 2H), 6.54 (d, J=8.6 Hz, 1H), 6.61 (s, 1H), 7.29 (s, 1H), 7.51 (dd, J=8.7, 2.4 Hz, 1H), 7.95-8.00 (m, 2H).

### Example Number 1202

### 2-Methyl-N-((6-(((1-methylpiperidin-4-yl)methyl)amino)pyridin-3-yl)methyl)-1H-pyrrolo[2,3-b]pyridin-4-amine

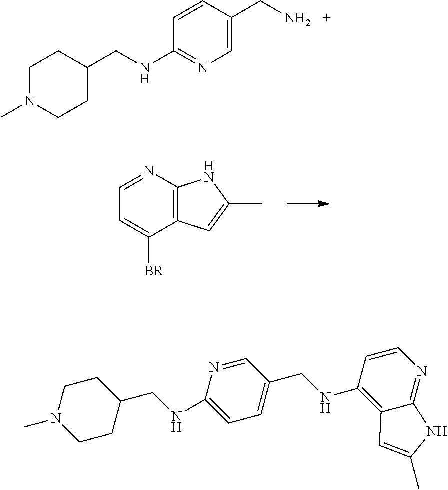

To a mixture of 5-(aminomethyl)-N-((1-methylpiperidin-4-yl)methyl)pyridin-2-amine (128 mg, 547 μmol), 4-chloro-2-methyl-1H-pyrrolo[2,3-b]pyridine (76.0 mg, 456 μmol) and BrettPhos Pd G3 (20.7 mg, 22.8 μmol) under N₂ (g) was added a solution of lithium bis(trimethylsilyl)amide (1M in THF) (1.09 mL, 1.09 mmol). The mixture heated at 70° C. for 6 h then left at rt for 12 h. AcOH (0.2 mL) and MeOH (1 mL) were added and after 5 min the mixture was diluted with MeOH (15 mL). The crude solution was loaded onto SCX and washed with MeOH. The product was eluted with 0.7M NH₃ in MeOH and the eluent concentrated. Crude product was purified by automated prep HPLC (mass directed 15-45% over 12.5 min in basic mobile phase) to obtain the product (105 mg, 61%) as a white solid.

[M+H]⁺=365.3

¹H NMR (500 MHz, DMSO) 1.13 (2H, qd, J=12.0, 3.9 Hz), 1.38-1.50 (1H, m), 1.64 (2H, d, J=10.8 Hz), 1.76 (2H, td, J=11.6, 2.5 Hz), 2.11 (3H, s), 2.29 (3H, s), 2.67-2.75 (2H, m), 3.07 (2H, t, J=6.3 Hz), 4.21 (2H, d, J=5.8 Hz), 6.07 (1H, d, J=5.5 Hz), 6.20 (1H, d, J=1.2 Hz), 6.38-6.45 (2H, m), 6.68 (1H, t, J=6.0 Hz), 7.33 (1H, dd, J=8.6, 2.4 Hz), 7.65 (1H, d, J=5.4 Hz), 7.94 (1H, d, J=2.4 Hz), 10.91 (1H, s)

### Example Number 1219

### N2-Methyl-N4-((6-(((1-methylpiperidin-4-yl)methyl)amino)pyridin-3-yl)methyl)pyridine-2,4-diamine

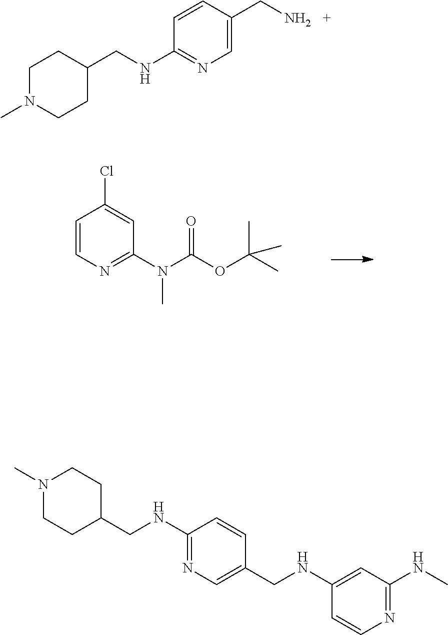

To a mixture of 5-(aminomethyl)-N-((1-methylpiperidin-4-yl)methyl)pyridin-2-amine (86.9 mg, 371 μmol), tert-butyl (4-chloropyridin-2-yl)(methyl)carbamate (75.0 mg, 309 μmol) and BrettPhosPd G3 (14.0 mg, 0.05 eq, 15.5 μmol) in THF (0.4 mL) was added a solution of lithium bis(trimethylsilyl)amide (1M in THF) (742 μL, 742 μmol). The mixture heated at 70° C. for 2 h. AcOH (0.2 mL) and MeOH (1 mL) were added to form a solution. This was stirred for 5 min then diluted with MeOH (15 mL). The crude solution was loaded onto SCX and washed with MeOH. The product was eluted with 0.7M NH₃ in MeOH and the eluent concentrated. The residue was dissolved in a mixture of DCM (3 mL) and TFA (1 mL) and the mixture stirred at rt for 18 h. The crude product was loaded onto SCX with MeOH and washed with MeOH. The product was eluted with 0.7M NH₃ in MeOH and the eluent concentrated. Flash chromatography (Silica, 0-45% (0.7M NH₃ in MeOH) in DCM) afforded the product (33 mg, 30%) as an off-white solid.

[M+H]⁺=341.3

¹H NMR (500 MHz, DMSO-d6) 1.09-1.19 (2H, m), 1.39-1.50 (1H, m), 1.64 (2H, d, J=10.9 Hz), 1.76 (2H, td, J=11.6, 2.5 Hz), 2.11 (3H, s), 2.65 (3H, d, J=4.9 Hz), 2.72 (2H, d, J=11.4 Hz), 3.08 (2H, t, J=6.3 Hz), 3.99 (2H, d, J=5.7 Hz), 5.50 (1H, d, J=2.0 Hz), 5.78 (1H, q, J=4.9 Hz), 5.85 (1H, dd, J=5.8, 2.0 Hz), 6.36 (1H, t, J=5.7 Hz), 6.40-6.47 (2H, m), 7.29 (1H, dd, J=8.6, 2.4 Hz), 7.49 (1H, d, J=5.8 Hz), 7.90 (1H, d, J=2.4 Hz)

### Example Number 1232

### 2-Methyl-N-((6-(((1-methylpiperidin-4-yl)methyl)amino)pyridin-3-yl)methyl)-1H-pyrrolo[2,3-c]pyridin-4-amine

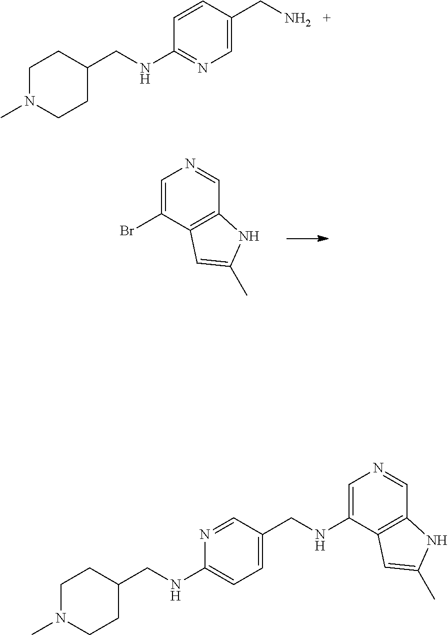

To a mixture of 5-(aminomethyl)-N-((1-methylpiperidin-4-yl)methyl)pyridin-2-amine (73.3 mg, 313 μmol), 4-bromo-2-methyl-1H-pyrrolo[2,3-c]pyridine (55.0 mg, 261 μmol) and BrettPhos Pd G3 (11.8 mg, 13.0 μmol) under N₂ (g) was added a solution of lithium bis(trimethylsilyl)amide (1M in THF) (625 μL, 625 μmol). The mixture heated at 70° C. for 1.5 h. AcOH (0.2 mL) and MeOH (1 mL) were added and after 5 min the mixture was diluted with MeOH (15 mL). The crude solution was loaded onto SCX and washed with MeOH. The product was eluted with 0.7M NH₃ in MeOH and the eluent concentrated. Crude product was purified by automated prep HPLC (mass directed 5-35% over 17.5 min in basic mobile phase) to obtain the product (27.5 mg, 29%) as a pale yellow solid.

[M+H]⁺=365.3

¹H NMR (500 MHz, DMSO-d6) 1.13 (2H, qd, J=3.8, 12.0 Hz), 1.38-1.50 (1H, m), 1.64 (2H, d, J=11.1 Hz), 1.76 (2H, td, J=2.5, 11.7 Hz), 2.11 (3H, s), 2.36 (3H, s), 2.71 (2H, d, J=11.6 Hz), 3.06 (2H, t, J=6.2 Hz), 4.20 (2H, d, J=6.0 Hz), 5.90 (1H, t, J=6.1 Hz), 6.32 (1H, s), 6.36-6.43 (2H, m), 7.30 (1H, s), 7.36 (1H, dd, J=2.4, 8.6 Hz), 7.94 (1H, s), 7.96 (1H, d, J=2.3 Hz), 10.95 (1H, s)

### Example Number 1274

### N5-((6-(3-(Difluoromethyl)-5,6-dihydro-[1,2,4]triazolo[4,3-a]pyrazin-7(8H)-yl)pyridin-3-yl)methyl)isoquinoline-1,5-diamine

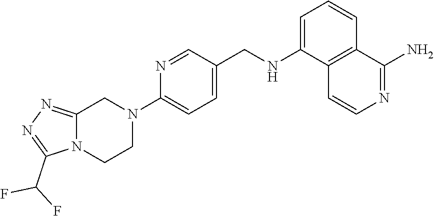 [SMILES: Nc1nccc2c(NCc3ccc(N4CCn5c(nnc5C(F)F)C4)nc3)cccc12]

### N5-((6-(3-(Difluoromethyl)-5,6-dihydro-[1,2,4]triazolo[4,3-a]pyrazin-7(8H)-yl)pyridin-3-yl)methyl)-N1-(2,4-dimethoxybenzyl)isoquinoline-1,5-diamine

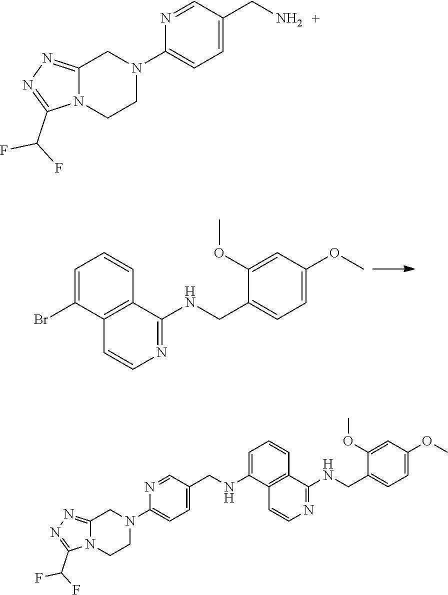 [SMILES: NCc1ccc(N2CCn3c(nnc3C(F)F)C2)nc1 ; COc1ccc(CNc2nccc3c(Br)cccc23)c(OC)c1 ; COc1ccc(CNc2nccc3c(NCc4ccc(N5CCn6c(nnc6C(F)F)C5)nc4)cccc23)c(OC)c1]

Following General Method 4, (6-(3-(difluoromethyl)-5,6-dihydro-[1,2,4]triazolo[4,3-a]pyrazin-7(8H)-yl)pyridin-3-yl)methanamine (945 mg, 3.37 mmol) was reacted with 5-bromo-N-(2,4-dimethoxybenzyl)isoquinolin-1-amine (1.20 g, 3.21 mmol) in the presence of CsCO₃ (2.09 g, 6.42 mmol) using BrettPhos Pd G4 (148 mg, 161 μmol) in 1,4-dioxane (13 mL). The mixture was diluted with EtOAc and concentrated onto silica. Flash chromatography (Silica, 0-6% (0.7M NH₃ in MeOH) in DCM) afforded the product (1.56 g, 68%) as a pale yellow foam.

[M+H]⁺=573.4

¹H NMR (500 MHz, DMSO-d6) 3.70 (3H, s), 3.82 (3H, s), 4.05 (2H, t, J=5.5 Hz), 4.19 (2H, t, J=5.5 Hz), 4.36 (2H, d, J=5.8 Hz), 4.58 (2H, d, J=5.7 Hz), 4.90 (2H, s), 6.38 (1H, dd, J=8.4, 2.4 Hz), 6.54 (1H, d, J=2.4 Hz), 6.57 (1H, d, J=7.8 Hz), 6.65 (1H, t, J=6.0 Hz), 7.01 (1H, d, J=8.3 Hz), 7.04 (1H, d, J=8.7 Hz), 7.14 (1H, d, J=6.2 Hz), 7.18 (1H, t, J=8.0 Hz), 7.23-7.47 (3H, m), 7.65 (1H, dd, J=8.7, 2.4 Hz), 7.74 (1H, d, J=6.1 Hz), 8.23 (1H, d, J=2.3 Hz)

### N5-((6-(3-(Difluoromethyl)-5,6-dihydro-[1,2,4]triazolo[4,3-a]pyrazin-7(8H)-yl)pyridin-3-yl)methyl)isoquinoline-1,5-diamine

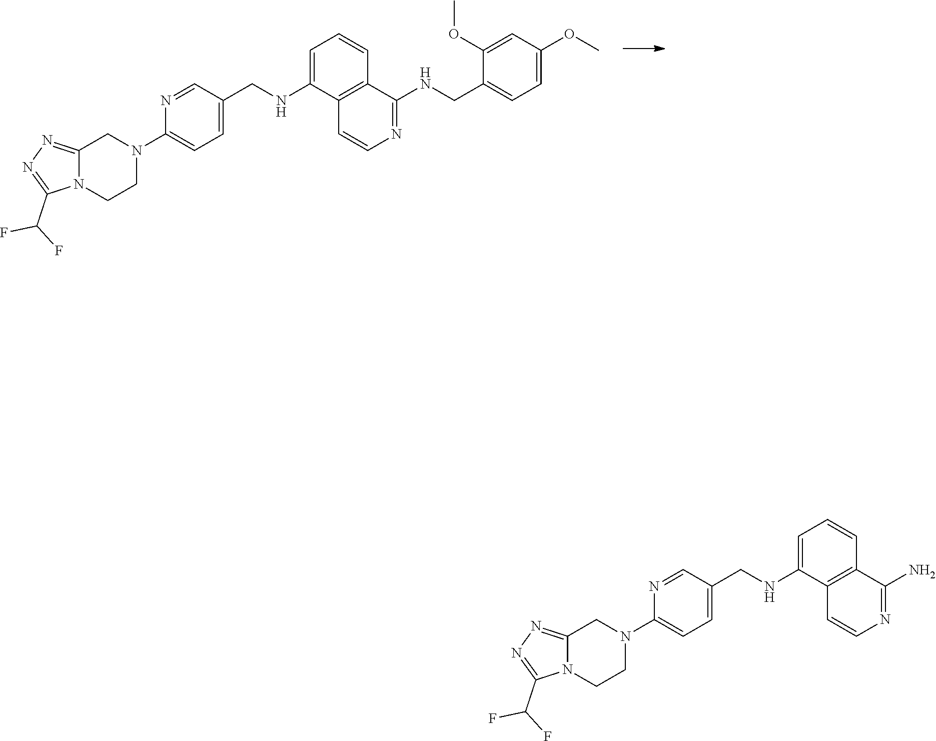 [SMILES: COc1ccc(CNc2nccc3c(NCc4ccc(N5CCn6c(nnc6C(F)F)C5)nc4)cccc23)c(OC)c1 ; Nc1nccc2c(NCc3ccc(N4CCn5c(nnc5C(F)F)C4)nc3)cccc12]

Deprotection of N5-((6-(3-(difluoromethyl)-5,6-dihydro-[1,2,4]triazolo[4,3-a]pyrazin-7(8H)-yl)pyridin-3-yl)methyl)-N1-(2,4-dimethoxybenzyl)isoquinoline-1,5-diamine (1.56 g, 2.48 mmol) was carried out using General Method 12. The mixture was diluted with MeCN (100 mL) and loaded onto SCX then washed with MeOH. The product was eluted with 0.7M NH₃ in MeOH and the eluent concentrated in vacuo. Flash chromatography (Silica, 0-13% (0.7M NH₃ in MeOH) in DCM) afforded the product which was slurried in a minimum quantity of MeCN for 1 h then filtered. The resultant solid was freeze-dried from 9:1 MeCN/H₂O (10 mL) to afford the product (903 mg, 85%) as a white solid.

[M+H]⁺=423.3

¹H NMR (500 MHz, DMSO-d6) 4.05 (2H, t, J=5.4 Hz), 4.19 (2H, t, J=5.4 Hz), 4.34 (2H, d, J=5.8 Hz), 4.90 (2H, s), 6.48 (2H, s), 6.54 (1H, d, J=7.7 Hz), 6.61 (1H, t, J=6.0 Hz), 7.04 (1H, d, J=8.7 Hz), 7.13 (1H, t, J=8.0 Hz),7.16 (1H, d, J=6.2 Hz), 7.30 (1H, d, J=8.3 Hz), 7.35 (1H, t, J=51.9 Hz), 7.64 (1H, dd, J=8.7, 2.4 Hz), 7.73 (1H, d, J=6.0 Hz), 8.22 (1H, d, J=2.3 Hz)

### Example Number 1299

### N5-((4-Chloro-6-(3-(trifluoromethyl)-5,6-dihydro-[1,2,4]triazolo[4,3-a]pyrazin-7(8H)-yl)pyridin-3-yl)methyl)isoquinoline-1,5-diamine

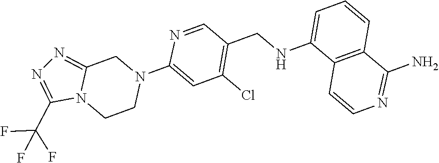 [SMILES: Nc1nccc2c(NCc3cnc(N4CCn5c(nnc5C(F)(F)F)C4)cc3Cl)cccc12]

### N5-((6-Bromo-4-chloropyridin-3-yl)methyl)-N1-(2,4-dimethoxybenzyl)isoquinoline-1,5-diamine

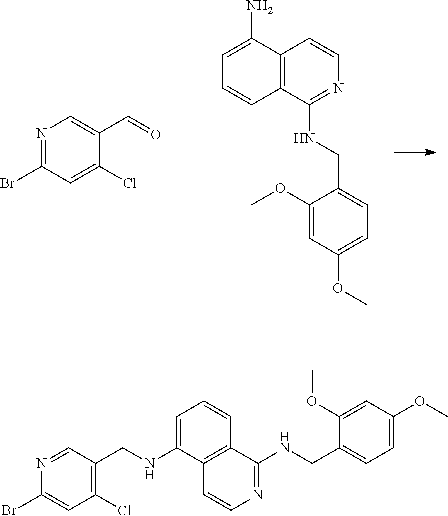

A mixture of 6-bromo-4-chloronicotinaldehyde (123 mg, 558 μmol) and N1-(2,4-dimethoxybenzyl)isoquinoline-1,5-diamine (115 mg, 372 μmol) in dichloroethane (5 mL) was treated with AcOH (44.6 mg, 743 μmol) and the mixture stirred at 65° C. for 20 h then at rt for 6 days. The mixture was partitioned between DCM (10 mL) and sat. NaHCO₃ (aq) (10 mL) and the organic layer collected. The aqueous layer was washed with further DCM (5 mL) and the combined organics concentrated in vacuo. The residue was dissolved in a mixture of EtOH (1.2 mL) and THF (2.0 mL) then treated with NaBH₄ (141 mg, 3.72 mmol). The mixture was stirred at rt for 2.5 h. Solvents were removed under vacuum and the residue partitioned between DCM (10 mL) and water (10 mL). The organic layer was collected with a phase separation cartridge and the aqueous extracted with further DCM (2×10 mL). The organic phases were combined. Flash chromatography (Silica, 0-60% EtOAc/Iso-Hexanes) afforded the product (140 mg, 69%) as a white foam.

[M+H]⁺=513.0/515.0/517.01

¹H NMR (DMSO, 500 MHz) δ 3.71 (3H, s), 3.82 (3H, s), 4.51 (2H, d, J=5.6 Hz), 4.59 (2H, d, J=5.7 Hz), 6.39 (1H, dd, J=8.4, 2.4 Hz), 6.48 (1H, d, J=7.7 Hz), 6.55 (1H, d, J=2.4 Hz), 6.71 (1H, t, J=5.8 Hz), 7.02 (1H, d, J=8.4 Hz), 7.15 (1H, d, J=6.1 Hz), 7.21 (1H, t, J=8.0 Hz), 7.45 (1H, t, J=5.8 Hz), 7.52 (1H, d, J=8.4 Hz), 7.77 (1H, d, J=6.0 Hz), 7.95 (1H, s), 8.26 (1H, s)

### N5-((4-Chloro-6-(3-(trifluoromethyl)-5,6-dihydro-[1,2,4]triazolo[4,3-a]pyrazin-7(8H)-yl)pyridin-3-yl)methyl)-N1-(2,4-dimethoxybenzyl)isoquinoline-1,5-diamine

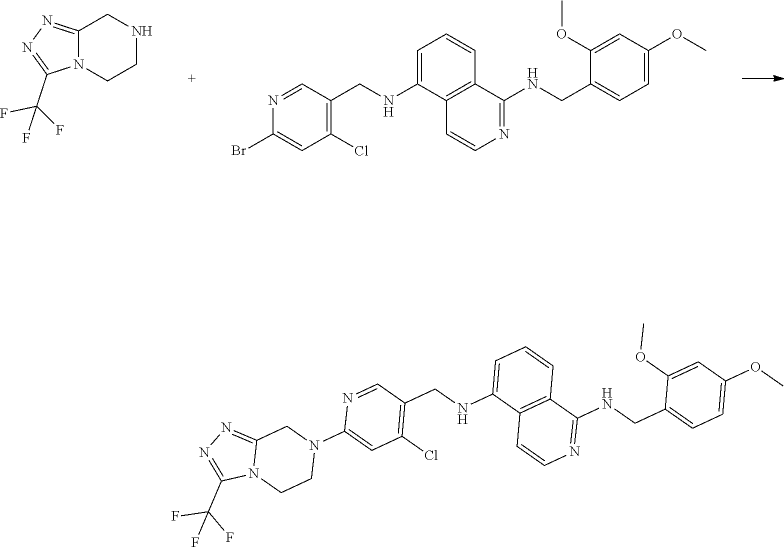 [SMILES: FC(F)(F)c1nnc2n1CCNC2 ; "+" ; COc1ccc(CNc2nccc3c(NCc4cnc(Br)cc4Cl)cccc23)c(OC)c1 ; COc1ccc(CNc2nccc3c(NCc4cnc(N5CCn6c(nnc6C(F)(F)F)C5)cc4Cl)cccc23)c(OC)c1]

Following General Method 4 (using Ruphos Pd G3 as catalyst) 3-(trifluoromethyl)-5,6,7,8-tetrahydro-[1,2,4]triazolo[4,3-a]pyrazine (10.9 mg, 56.8 μmol) was reacted with N5-((6-bromo-4-chloropyridin-3-yl)methyl)-N1-(2,4-dimethoxybenzyl)isoquinoline-1,5-diamine (24.3 mg, 47.3 μmol) in the presence of RuPhos (2.21 mg, 4.73 μmol) and CsCO₃ (46.2 mg, 142 μmol) in THF (0.75 mL) at 80° C. for 18 h. On cooling, the mixture was partitioned between EtOAc (10 mL) and water (10 mL). The aqueous layer was extracted with further EtOAc (10 mL) and the combined organics washed with brine (10 mL), dried (MgSO₄), filtered and concentrated. Flash chromatography (Silica, 0-3% (0.7M NH₃ in MeOH) in DCM) to afforded the product (6.8 mg, 21%) as an orange solid.

[M+H]⁺=625.5/627.4

### N5-((4-Chloro-6-(3-(trifluoromethyl)-5,6-dihydro-[1,2,4]triazolo[4,3-a]pyrazin-7(8H)-yl)pyridin-3-yl)methyl)isoquinoline-1,5-diamine

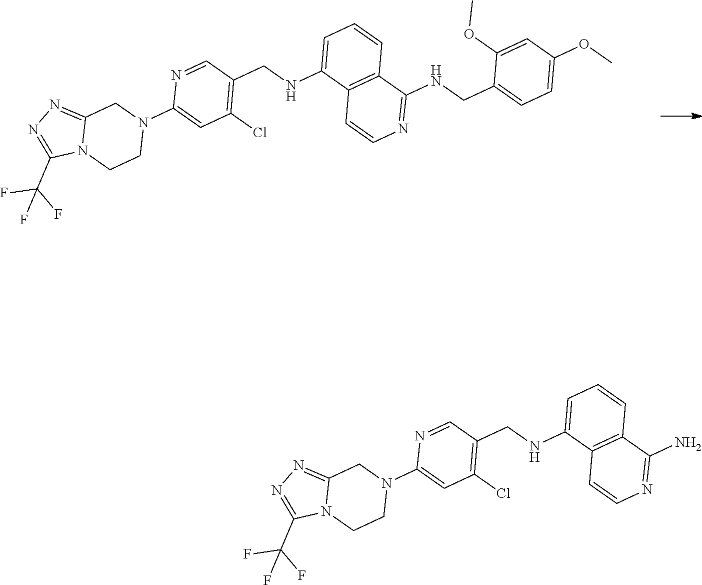 [SMILES: COc1ccc(CNc2nccc3c(NCc4cnc(N5CCn6c(nnc6C(F)(F)F)C5)cc4Cl)cccc23)c(OC)c1 ; Nc1nccc2c(NCc3cnc(N4CCn5c(nnc5C(F)(F)F)C4)cc3Cl)cccc12]

Deprotection of N5-((4-chloro-6-(3-(trifluoromethyl)-5,6-dihydro-[1,2,4]triazolo[4,3-a]pyrazin-7(8H)-yl)pyridin-3-yl)methyl)-N1-(2,4-dimethoxybenzyl)isoquinoline-1,5-diamine (6.2 mg, 8.9 μmol) was carried out using General Method 12. The crude product was loaded onto SCX with MeCN and washed with MeOH. The product was eluted with 0.7M NH in MeOH and the eluent concentrated. Flash chromatography (Silica, 0-6% (0.7M NH₃ in MeOH) in DCM) afforded the product (3.5 mg, 78%) as a light yellow solid.

[M+H]⁺=475.4/477.4

¹H NMR (DMSO, 500 MHz) δ 4.10 (2H, t, J=5.5 Hz), 4.23 (2H, t, J=5.4 Hz), 4.42 (2H, d, J=5.6 Hz), 4.99 (2H, s), 6.54 (1H, d, J=7.8 Hz), 6.59 (1H, t, J=5.6 Hz), 6.76 (2H, br s), 7.16-7.24 (2H, m), 7.28 (1H, s), 7.39 (1H, d, J=8.3 Hz), 7.73 (1H, d, J=6.2 Hz), 8.10 (1H, s)

### Example Number 2256

### N5-(2-Fluoro-4-(3-(trifluoromethyl)-5,6-dihydro-[1,2,4]triazolo[4,3-a]pyrazin-7(8H)-yl)benzyl)isoquinoline-1,5-diamine

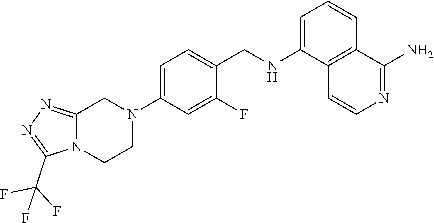 [SMILES: Nc1nccc2c(NCc3ccc(N4CCn5c(nnc5C(F)(F)F)C4)cc3F)cccc12]

### (2-Fluoro-4-(3-(trifluoromethyl)-5,6-dihydro-[1,2,4]triazolo[4,3-a]pyrazin-7(8H)-yl)phenyl)methanamine

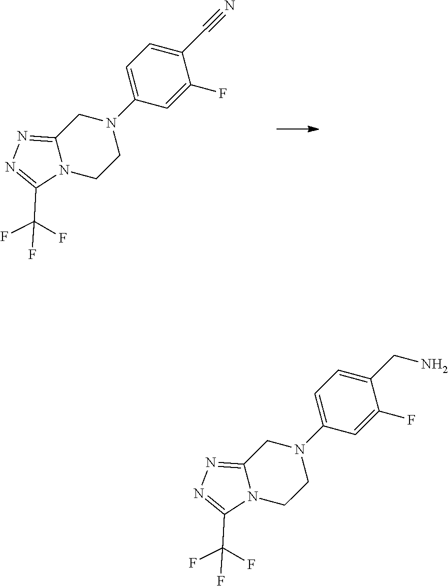 [SMILES: N#Cc1ccc(N2CCn3c(nnc3C(F)(F)F)C2)cc1F ; NCc1ccc(N2CCn3c(nnc3C(F)(F)F)C2)cc1F]

Reduction of 2-fluoro-4-(3-(trifluoromethyl)-5,6-dihydro-[1,2,4]triazolo[4,3-a]pyrazin-7(8H)-yl)benzonitrile (278 mg, 893 μmol) was carried out using General Method 3a. Flash chromatography (silica, 12 g cartridge, eluted with 0-20% (0.7M NH₃ in MeOH) in DCM afforded the product (249 mg, 84%) as a white solid

[M+H]⁺=316.7

¹H NMR (DMSO, 500 MHz) δ 3.64 (2H, s), 3.80 (2H, t, J=5.5 Hz), 4.25 (2H, t, J=5.5 Hz), 4.68 (2H, s), 6.88-6.96 (2H, m), 7.33 (1H, t, J=8.7 Hz)

### Methyl (5-((2-fluoro-4-(3-(trifluoromethyl)-5,6-dihydro-[1,2,4]triazolo[4,3-a]pyrazin-7(8H)-yl)benzyl)amino)isoquinolin-1-yl)carbamate

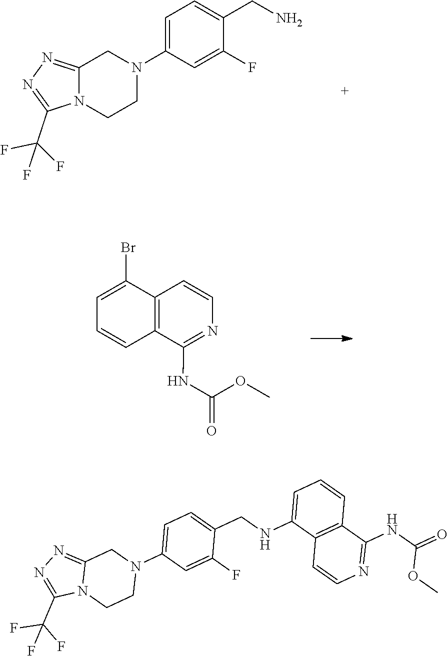 [SMILES: NCc1ccc(N2CCn3c(nnc3C(F)(F)F)C2)cc1F ; "+" ; COC(=O)Nc1nccc2c(Br)cccc12 ; COC(=O)Nc1nccc2c(NCc3ccc(N4CCn5c(nnc5C(F)(F)F)C4)cc3F)cccc12]

Following General Method 4, (2-fluoro-4-(3-(trifluoromethyl)-5,6-dihydro-[1,2,4]triazolo[4,3-a]pyrazin-7(8H)-yl)phenyl)methanamine (136 mg, 430 μmol) was reacted with methyl (5-bromoisoquinolin-1-yl)carbamate (110 mg, 391 μmol) and NaOtBu (2M in THF) (391 μL, 783 μmol) in 1,4-dioxane (2 mL) at 75° C. for 4 h. The reaction mixture was cooled to rt and combined with a previous batch. The resulting mixture was diluted with EtOAc, filtered over Celite® and washed with further EtOAc. Flash chromatography (silica, 12 g cartridge, eluted with 0-20% (0.7M NH₃ in MeOH) in DCM) afforded the product (32% overall yield) as a yellow oil.

[M+H]⁺=516.3

### N5-(2-Fluoro-4-(3-(trifluoromethyl)-5,6-dihydro-[1,2,4]triazolo[4,3-a]pyrazin-7(8H)-yl)benzyl)isoquinoline-1,5-diamine

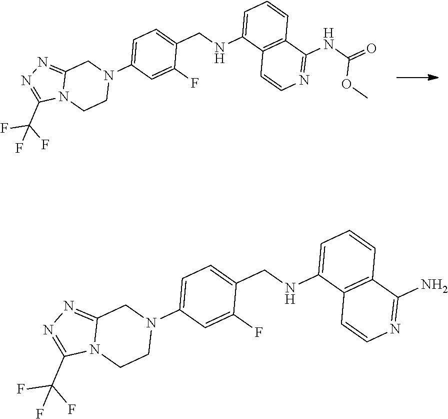 [SMILES: COC(=O)Nc1nccc2c(NCc3ccc(N4CCn5c(nnc5C(F)(F)F)C4)cc3F)cccc12 ; Nc1nccc2c(NCc3ccc(N4CCn5c(nnc5C(F)(F)F)C4)cc3F)cccc12]

A solution of methyl (5-((2-fluoro-4-(3-(trifluoromethyl)-5,6-dihydro-[1,2,4]triazolo[4,3-a]pyrazin-7(8H)-yl)benzyl)amino)isoquinolin-1-yl)carbamate (104.0 mg, 147.3 μmol) in MeOH (2 mL) was treated with NaOH(aq) (2M) (280 μL, 560 μmol) and the mixture stirred at 65° C. for 17 h. The mixture was cooled to rt, diluted with EtOAc, and washed with brine. The organic layer was dried over MgSO₄, filtered and concentrated in vacuo. Flash chromatography (Silica, 0-20% (0.7M NH₃ inMeOH) in DCM) followed by lyophilisation afforded the product (57 mg, 81%) as a beige solid.

[M+H]⁺=458.2

¹H NMR (DMSO, 500 MHz) δ 3.78 (2H, t, J=5.5 Hz), 4.24 (2H, t, J=5.5 Hz), 4.39 (2H, d, J=5.8 Hz), 4.67 (2H, s), 6.45-6.51 (3H, m), 6.59 (1H, t, J=6.0 Hz), 6.86 (1H, dd, J=8.7, 2.5 Hz), 7.00 (1H, dd, J=13.5, 2.5 Hz), 7.14 (1H, t, J=8.0 Hz), 7.18 (1H, d, J=6.1 Hz), 7.22 (1H, t, J=8.8 Hz), 7.31 (1H, d, J=8.4 Hz), 7.74 (1H, d, J=6.1 Hz)

### Example Number 9002

### N5-((5-(3-(trifluoromethyl)-5,6-dihydro-[1,2,4]triazolo[4,3-a]pyrazin-7(8H)-yl)pyrazin-2-yl)methyl)isoquinoline-1,5-diamine

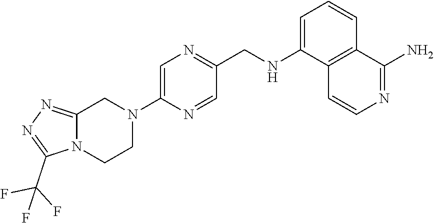 [SMILES: Nc1nccc2c(NCc3cnc(N4CCn5c(nnc5C(F)(F)F)C4)cn3)cccc12]

### 5-(3-(Trifluoromethyl)-5,6-dihydro-[1,2,4]triazolo[4,3-a]pyrazin-7(8H)-yl)pyrazine-2-carbonitrile

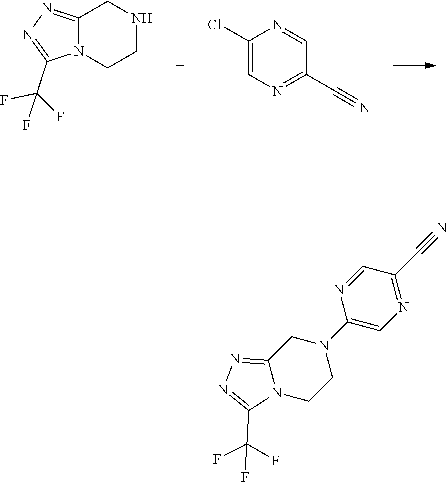

A solution of 3-(trifluoromethyl)-5,6,7,8-tetrahydro-[1,2,4]triazolo[4,3-a]pyrazine (361 mg, 1.88 mmol) and 5-chloropyrazine-2-carbonitrile (250 mg, 1.79 mmol) in anhydrous MeCN (3 mL) was treated with DIPEA (640 μL, 3.67 mmol) and the mixture heated at 140° C. in a microwave reactor for 6 h. Solvents were removed in vacuo. Flash chromatography (Silica, 0-2.5% (0.7M NH₃ in MeOH) in DCM) afforded the product (464 mg, 87%) as a tan solid.

[M−H]⁻=294.2

¹H NMR (DMSO, 500 MHz) δ 4.30 (4H, s), 5.22 (2H, s), 8.65 (1H, d, J=1.4 Hz), 8.69 (1H, d, J=1.4 Hz)

### (5-(3-(trifluoromethyl)-5,6-dihydro-[1,2,4]triazolo[4,3-a]pyrazin-7(8H)-yl)pyrazin-2-yl)methanamine

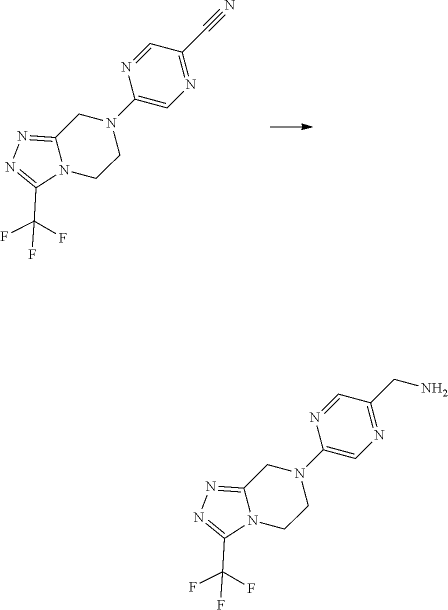 [SMILES: N#Cc1cnc(N2CCn3c(nnc3C(F)(F)F)C2)cn1 ; NCc1cnc(N2CCn3c(nnc3C(F)(F)F)C2)cn1]

5-(3-(Trifluoromethyl)-5,6-dihydro-[1,2,4]triazolo[4,3-a]pyrazin-7(8H)-yl)pyrazine-2-carbonitrile (211 mg, 716 μmol) reduced according to General Method 3a over 4 h using a Raney-Ni cartridge. Solvents were removed in vacuo to afford the product (203 mg, 90%) as a brown glass. [M-NH]⁺=283.3

¹H NMR (DMSO, 500 MHz) δ 2.31 (2H, brs), 3.73 (2H, s), 4.14 (2H, t, J=5.5 Hz), 4.28 (2H, t, J=5.4 Hz), 5.03 (2H, s), 8.20 (1H, d, J=1.5 Hz), 8.47 (1H, d, J=1.5 Hz)

### Methyl (5-(((5-(3-(trifluoromethyl)-5,6-dihydro-[1,2,4]triazolo[4,3-a]pyrazin-7(8H)-yl)pyrazin-2-yl)methyl)amino)isoquinolin-1-yl)carbamate

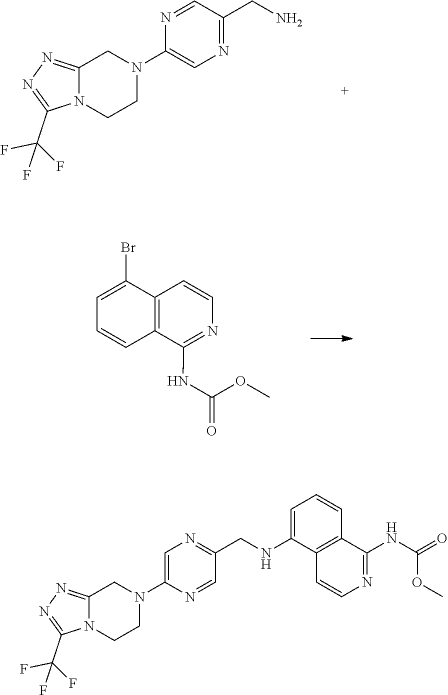 [SMILES: NCc1cnc(N2CCn3c(nnc3C(F)(F)F)C2)cn1 ; "+" ; COC(=O)Nc1nccc2c(Br)cccc12 ; COC(=O)Nc1nccc2c(NCc3cnc(N4CCn5c(nnc5C(F)(F)F)C4)cn3)cccc12]

Following General Method 4, (5-(3-(trifluoromethyl)-5,6-dihydro-[1,2,4]triazolo[4,3-a]pyrazin-7(8H)-yl)pyrazin-2-yl)methanamine (100 mg, 334 μmol) was reacted with methyl (5-bromoisoquinolin-1-yl)carbamate (93.9 mg, 334 μmol) and NaOtBu (64 mg, 668 μmol) in anhydrous THF (2.2 mL) at 65° C. for 22 h. After cooling the mixture was partitioned between EtOAc (10 mL) and water (10 mL). The aqueous was extracted with EtOAc (2×10 mL) and the combined organics washed with brine (10 mL), dried (MgSO₄), filtered and concentrated. Flash chromatography (Silica, 0-9% (0.7M NH₃ in MeOH) in DCM) afforded the product (90.5 mg, 43%) as a pale yellow solid.

[M+H]⁺=500.4

¹H NMR (DMSO, 500 MHz) δ 3.65 (3H, s), 4.13 (2H, t, J=5.4 Hz), 4.26 (2H, t, J=5.5 Hz), 4.51 (2H, d, J=5.9 Hz), 5.02 (2H, s), 6.67 (1H, d, J=7.6 Hz), 7.10 (1H, t, J=6.0 Hz), 7.24 (1H, d, J=8.4 Hz), 7.31 (1H, t, J=8.0 Hz), 7.95 (1H, d, J=6.0 Hz), 8.20-8.24 (2H, m), 8.51 (1H, d, J=1.5 Hz), 9.85 (1H, s)

### N5-((5-(3-(Trifluoromethyl)-5,6-dihydro-[1,2,4]triazolo[4,3-a]pyrazin-7(8H)-yl)pyrazin-2-yl)methyl)isoquinoline-1,5-diamine

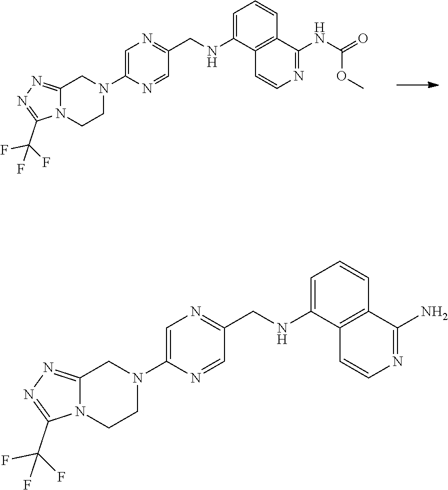 [SMILES: COC(=O)Nc1nccc2c(NCc3cnc(N4CCn5c(nnc5C(F)(F)F)C4)cn3)cccc12 ; Nc1nccc2c(NCc3cnc(N4CCn5c(nnc5C(F)(F)F)C4)cn3)cccc12]

Deprotection of methyl (5-(((5-(3-(trifluoromethyl)-5,6-dihydro-[1,2,4]triazolo[4,3-a]pyrazin-7(8H)-yl)pyrazin-2-yl)methyl)amino)isoquinolin-1-yl)carbamate (88.0 mg, 138 μmol) was performed using General Method 14a. The mixture was partitioned between EtOAc (15 mL) and sat. NH₄Cl(aq) (15 mL). The aqueous layer was extracted with EtOAc (7 mL) and the combined organics washed with brine (10 mL), dried (MgSO₄), filtered and concentrated. Flash chromatography (Silica, 0-10% (0.7M NH₃ in MeOH) in DCM) afforded the product (24 mg, 39%) as a pale yellow solid

[M+H]⁺=442.2

¹H NMR (DMSO, 500 MHz) δ 4.13 (2H, t, J=5.4 Hz), 4.26 (2H, t, J=5.4 Hz), 4.46 (2H, d, J=5.7 Hz), 5.02 (2H, s), 6.50 (2H, s), 6.55 (1H, d, J=7.7 Hz), 6.69 (1H, t, J=6.0 Hz), 7.10-7.19 (2H, m), 7.32 (1H, d, J=8.3 Hz), 7.74 (1H, d, J=6.1 Hz), 8.17 (1H, d, J=1.4 Hz), 8.51 (1H, d, J=1.5 Hz)

### Example Number 9004

### N5-((6-(3-(Trifluoromethyl)-5,6-dihydro-[1,2,4]triazolo[4,3-a]pyrazin-7(8H)-yl)pyridazin-3-yl)methyl)isoquinoline-1,5-diamine

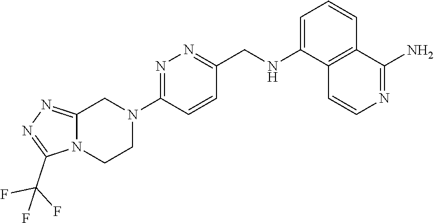 [SMILES: Nc1nccc2c(NCc3ccc(N4CCn5c(nnc5C(F)(F)F)C4)nn3)cccc12]

### 6-(3-(Trifluoromethyl)-5,6-dihydro-[1,2,4]triazolo[4,3-a]pyrazin-7(8H)-yl)pyridazine-3-carbonitrile

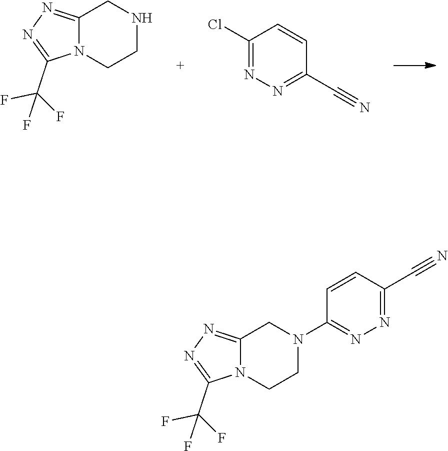

A solution of 3-(trifluoromethyl)-5,6,7,8-tetrahydro-[1,2,4]triazolo[4,3-a]pyrazine (361 mg, 1.88 mmol) and 6-chloropyridazine-3-carbonitrile (250 mg, 1.79 mmol) in anhydrous MeCN (3 mL) was treated with DIPEA (475 mg, 3.67 mmol) and the mixture heated at 140° C. in a microwave reactor for 3 h. Solvents were removed in vacuo. The residue was triturated from a minimum quantity of MeCN and filtered to afford the product (406 mg, 76%) as a light beige solid.

[M+H]⁺=296.3

¹H NMR (DMSO, 500 MHz) δ 4.27-4.37 (4H, m), 5.23 (2H, s), 7.62 (1H, d, J=9.7 Hz), 8.04 (1H, d, J=9.6 Hz)

### (6-(3-(Trifluoromethyl)-5,6-dihydro-[1,2,4]triazolo[4,3-a]pyrazin-7(8H)-yl)pyridazin-3-yl)methanamine

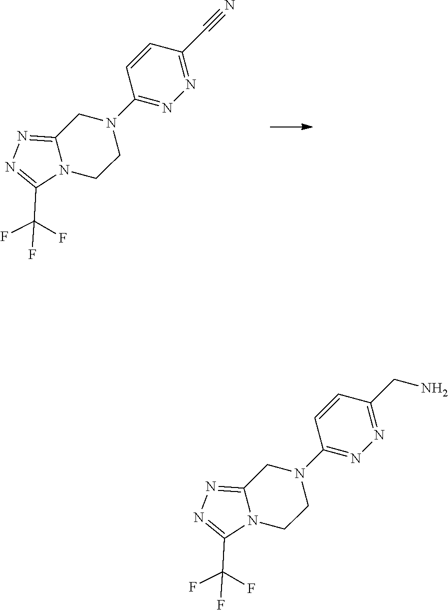 [SMILES: N#Cc1ccc(N2CCn3c(nnc3C(F)(F)F)C2)nn1 ; NCc1ccc(N2CCn3c(nnc3C(F)(F)F)C2)nn1]

6-(3-(Trifluoromethyl)-5,6-dihydro-[1,2,4]triazolo[4,3-a]pyrazin-7(8H)-yl)pyridazine-3-carbonitrile (197 mg, 667 μmol) was reduced using General Method 3a over 24 h using a Raney-Ni cartridge. Solvents were removed in vacuo. Flash chromatography (Silica, 0-18% (0.7M NH₃ in MeOH) in DCM) afforded the product (147 mg, 52%) as a white solid.

[M+H]⁺=300.3

¹H NMR (DMSO, 500 MHz) δ 2.30 (2H, brs), 3.84 (2H, s), 4.16 (2H, t, J=5.4 Hz), 4.28 (2H, t, J=5.4 Hz), 5.07 (2H, s), 7.53 (1H, d, J=9.4 Hz), 7.58 (1H, d, J=9.4 Hz)

### Methyl (5-(((6-(3-(trifluoromethyl)-5,6-dihydro-[1,2,4]triazolo[4,3-a]pyrazin-7(8H)-yl)pyridazin-3-yl)methyl)amino)isoquinolin-1-yl)carbamate

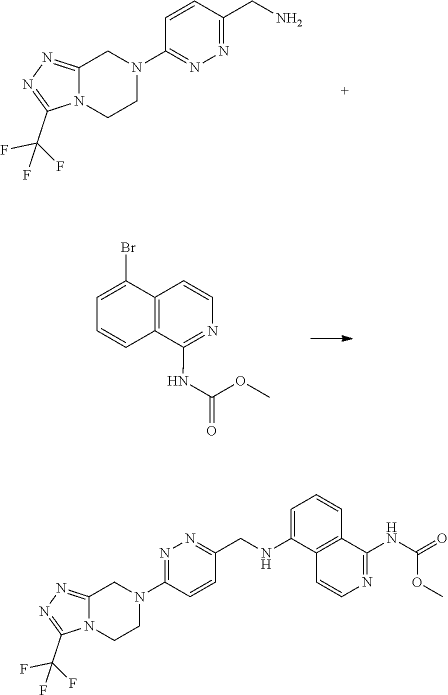 [SMILES: NCc1ccc(N2CCn3c(nnc3C(F)(F)F)C2)nn1 ; "+" ; COC(=O)Nc1nccc2c(Br)cccc12 ; COC(=O)Nc1nccc2c(NCc3ccc(N4CCn5c(nnc5C(F)(F)F)C4)nn3)cccc12]

Following General Method 4, (6-(3-(trifluoromethyl)-5,6-dihydro-[1,2,4]triazolo[4,3-a]pyrazin-7(8H)-yl)pyridazin-3-yl)methanamine (100 mg, 334 μmol) was reacted with methyl (5-bromoisoquinolin-1-yl)carbamate (93.9 mg, 334 μmol) and NaOtBu (64 mg, 668 μmol) in anhydrous THF (2.2 mL) at 65° C. for 22 h. The mixture was partitioned between EtOAc (10 mL) and water (10 mL). The aqueous was extracted with EtOAc (2×10 mL) and the combined organics washed with brine (10 mL), dried (MgSO₄), filtered and concentrated. Flash chromatography (Silica, 0-10% (0.7M NH₃ in MeOH) in DCM) afforded the product (63 mg, 33%) as a pale yellow solid.

[M+H]⁺=500.4

¹H NMR (DMSO, 500 MHz) δ 3.65 (3H, s), 4.16 (2H, t, J=5.4 Hz), 4.28 (2H, t, J=5.4 Hz), 4.63 (2H, d, J=5.9 Hz), 5.07 (2H, s), 6.66 (1H, d, J=7.6 Hz), 7.20 (1H, t, J=6.1 Hz), 7.25 (1H, d, J=8.4 Hz), 7.30 (1H, t, J=8.0 Hz), 7.44-7.52 (2H, m), 7.94 (1H, d, J=6.0 Hz), 8.23 (1H, d, J=6.0 Hz), 9.86 (1H, s)

### N5-((6-(3-(Trifluoromethyl)-5,6-dihydro-[1,2,4]triazolo[4,3-a]pyrazin-7(8H)-yl)pyridazin-3-yl)methyl)isoquinoline-1,5-diamine

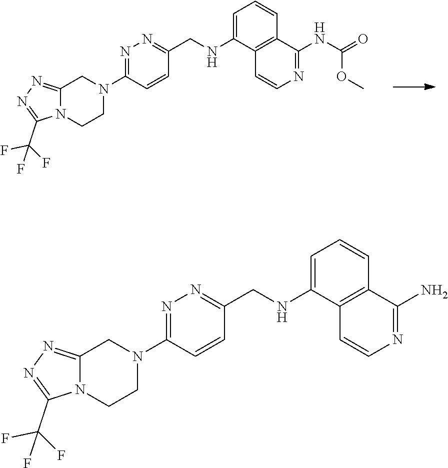 [SMILES: COC(=O)Nc1nccc2c(NCc3ccc(N4CCn5c(nnc5C(F)(F)F)C4)nn3)cccc12 ; Nc1nccc2c(NCc3ccc(N4CCn5c(nnc5C(F)(F)F)C4)nn3)cccc12]

Deprotection of methyl (5-(((6-(3-(trifluoromethyl)-5,6-dihydro-[1,2,4]triazolo[4,3-a]pyrazin-7(8H)-yl)pyridazin-3-yl)methyl)amino)isoquinolin-1-yl)carbamate (60.0 mg, 106 μmol) was performed using General Method 14a. The mixture was partitioned between EtOAc (15 mL) and sat. NH₄Cl(aq) (15 mL). The aqueous layer was extracted with EtOAc (2×7 mL) and the combined organics washed with brine (10 mL), dried (MgSO₄), filtered and concentrated. Flash chromatography (Silica, 0-9% (0.7M NH₃ in MeOH) in DCM) afforded the product (27 mg 55%) as a pale yellow solid

[M+H]⁺=442.2

¹H NMR (DMSO, 500 MHz) δ 4.16 (2H, t, J=5.4 Hz), 4.28 (2H, t, J=5.4 Hz), 4.58 (2H, d, J=5.9 Hz), 5.07 (2H, s), 6.51 (2H, s), 6.55 (1H, d, J=7.7 Hz), 6.82 (1H, t, J=6.1 Hz), 7.09-7.18 (2H, m), 7.32 (1H, d, J=8.3 Hz), 7.42-7.51 (2H, m), 7.75 (1H, d, J=6.1 Hz)

TABLE 11
Compound Names

| Example | |
|---|---|
| Number | Name |
| 1001 | 6-N-({6-[(1-methylpiperidin-4-yl)oxy]pyridin-3-yl}methyl)isoquinoline-1,6-diamine |
| 1002 | 5-N-({4-[(1-methylpiperidin-4-yl)oxy]phenyl}methyl)isoquinoline-1,5-diamine |
| 1003 | 7-N-({4-[(1-methylpiperidin-4-yl)oxy]phenyl}methyl)isoquinoline-1,7-diamine |
| 1004 | 3-chloro-N-(4-((1-methylpiperidin-4-yl)oxy)benzyl)-1H-pyrrolo[2,3-b]pyridin-5-amine |
| 1005 | 6-N-({6-[(1-methylpiperidin-4-yl)methoxy]pyridin-3-yl}methyl)isoquinoline-1,6-diamine |
| 1006 | 6-N-[(6-{5H,6H,7H,8H-imidazo[1,2-a]pyridin-7-ylmethoxy}pyridin-3- |
| | yl)methyl]isoquinoline-1,6-diamine |
| 1007 | 6-N-({4-[(4-methylpiperazin-1-yl)methyl]phenyl}methyl)isoquinoline-1,6-diamine |
| 1008 | N-({6-[(1-methylpiperidin-4-yl)methoxy]pyridin-3-yl}methyl)isoquinolin-6-amine |
| 1009 | 5-N-({6-[(1-methylpiperidin-4-yl)methoxy]pyridin-3-yl}methyl)isoquinoline-1,5-diamine |
| 1010 | 6-N-({6-[(1-ethylpiperidin-4-yl)methoxy]pyridin-3-yl}methyl)isoquinoline-1,6-diamine |
| 1011 | 6-[(4-{[(1-methylpiperidin-4-yl)oxy]methyl}phenyl)methoxy]isoquinolin-1-amine |
| 1012 | 6-N-({6-[(1-isopropylpiperidin-4-yl)methoxy]pyridin-3-yl}methyl)isoquinoline-1,6- |
| | diamine |
| 1013 | 6-N-({2-[(1-methylpiperidin-4-yl)methoxy]pyrimidin-5-yl}methyl)isoquinoline-1,6- |
| | diamine |
| 1014 | 6-N-{[6-(pyridin-4-ylmethoxy)pyridin-3-yl]methyl}isoquinoline-1,6-diamine |
| 1015 | 4-{[(5-{[(1-aminoisoquinolin-6-yl)amino]methyl}pyridin-2-yl)oxy]methyl}-1- |
| | methylpiperidin-2-one |
| 1016 | 6-N-{[6-(piperidin-4-ylmethoxy)pyridin-3-yl]methyl}isoquinoline-1,6-diamine |
| 1017 | 1-(4-{[(5-{[(1-aminoisoquinolin-6-yl)amino]methyl}pyridin-2-yl)oxy]methyl}piperidin-1- |
| | yl)-2-methylpropan-2-ol |
| 1018 | 6-(2-{6-[(1-methylpiperidin-4-yl)methoxy]pyridin-3-yl}ethyl)isoquinolin-1-amine |
| 1019 | 5-(2-{6-[(1-methylpiperidin-4-yl)methoxy]pyridin-3-yl}ethyl)isoquinolin-1-amine |
| 1020 | 3-chloro-N-((6-((1-methylpiperidin-4-yl)methoxy)pyridin-3-yl)methyl)-1H-pyrrolo[2,3- |
| | b]pyridin-5-amine |
| 1021 | 7-N-({6-[(1-methylpiperidin-4-yl)methoxy]pyridin-3-yl}methyl)quinazoline-4,7-diamine |
| 1022 | N8-[[6-[(1-methyl-4-piperidyl)methoxy]-3-pyridyl]methyl]quinazoline-4,8-diamine |
| 1023 | 6-N-({6-[(7R*)-5H,6H,7H,8H-imidazo[1,2-a]pyridin-7-ylmethoxy]pyridin-3- |
| | yl}methyl)isoquinoline-1,6-diamine |
| 1024 | 6-N-({6-[(7S*)-5H,6H,7H,8H-imidazo[1,2-a]pyridin-7-ylmethoxy]pyridin-3- |
| | yl}methyl)isoquinoline-1,6-diamine |
| 1025 | 6-N-({6-[3-(1-methylimidazol-2-yl)propoxy]pyridin-3-yl}methyl)isoquinoline-1,6-diamine |
| 1026 | {6-[({6-[(1-methylpiperidin-4-yl)methoxy]pyridin-3-yl}methyl)amino]isoquinolin-4- |
| | yl}methanol |
| 1027 | 6-N-({2-methoxy-6-[(1-methylpiperidin-4-yl)methoxy]pyridin-3-yl}methyl)isoquinoline- |
| | 1,6-diamine |
| 1028 | 6-N-[(6-{[2-(trifluoromethyl)-5H,6H,7H,8H-imidazo[1,2-a]pyridin-7-yl]methoxy}pyridin- |
| | 3-yl)methyl]isoquinoline-1,6-diamine |
| 1029 | 5-N-({6-[(7R*)-5H,6H,7H,8H-imidazo[1,2-a]pyridin-7-ylmethoxy]pyridin-3- |
| | yl}methyl)isoquinoline-1,5-diamine |
| 1030 | 5-N-({6-[(7S*)-5H,6H,7H,8H-imidazo[1,2-a]pyridin-7-ylmethoxy]pyridin-3- |
| | yl}methyl)isoquinoline-1,5-diamine |
| 1031 | 6-N-({6-[3-(imidazol-1-yl)propoxy]pyridin-3-yl}methyl)isoquinoline-1,6-diamine |
| 1032 | 6-N-({6-[2-(1-methylpiperidin-4-yl)ethoxy]pyridin-3-yl}methyl)isoquinoline-1,6-diamine |
| 1033 | N6-((6-(((1-methylpiperidin-4-yl)methyl)amino)pyridin-3-yl)methyl)isoquinoline-1,6- |
| | diamine |
| 1034 | 5-N-{[2-fluoro-4-(morpholin-4-ylmethyl)phenyl]methyl}isoquinoline-1,5-diamine |
| 1035 | N5-((2-(3-(1-methyl-1H-imidazol-2-yl)propoxy)pyrimidin-5-yl)methyl)isoquinoline-1,5- |
| | diamine |
| 1036 | N5-(2-fluoro-4-((6-isopropyl-2,6-diazaspiro[3.3]heptan-2-yl)methyl)benzyl)isoquinoline- |
| | 1,5-diamine |
| 1037 | 6-N-{[2-fluoro-4-({6-isopropyl-2,6-diazaspiro[3.3]heptan-2- |
| | yl}methyl)phenyl]methyl}isoquinoline-1,6-diamine |
| 1038 | 4-chloro-6-N-[(2-{[3-(1-methylimidazol-2-yl)propyl]amino}pyrimidin-5- |
| | yl)methyl]isoquinoline-1,6-diamine |
| 1039 | 3-[(5-{[(1-aminoisoquinolin-5-yl)amino]methyl}pyrimidin-2-yl)amino]-1-(pyrrolidin-1- |
| | yl)propan-1-one |
| 1040 | 3-[(5-{[(1-amino-4-chloroisoquinolin-6-yl)amino]methyl}pyrimidin-2-yl)amino]-1- |
| | (pyrrolidin-1-yl)propan-1-one |
| 1041 | 4-chloro-6-N-[(6-{5H,6H,7H,8H-imidazo[1,2-a]pyridin-7-ylmethoxy}pyridin-3- |
| | yl)methyl]isoquinoline-1,6-diamine |
| 1042 | 5-N-[(2-{5H,6H,7H,8H-imidazo[1,2-a]pyridin-7-ylmethoxy}pyrimidin-5- |
| | yl)methyl]isoquinoline-1,5-diamine |
| 1043 | 4-chloro-6-N-{[2-({5H,6H,7H,8H-imidazo[1,2-a]pyridin-7-ylmethyl}amino)pyrimidin-5- |
| | yl]methyl}isoquinoline-1,6-diamine |
| 1044 | 4-chloro-6-N-{[6-({5H,6H,7H,8H-imidazo[1,2-a]pyridin-7-ylmethyl}amino)pyridin-3- |
| | yl]methyl}isoquinoline-1,6-diamine |
| 1049 | 5-N-[(6-{5H,6H,7H,8H-imidazo[1,2-a]pyridin-7-yloxy}pyridin-3-yl)methyl]isoquinoline- |
| | 1,5-diamine |
| 1050 | 4-chloro-6-N-[(6-{5H,6H,7H,8H-imidazo[1,2-a]pyridin-7-yloxy}pyridin-3- |
| | yl)methyl]isoquinoline-1,6-diamine |
| 1052 | 4-chloro-6-N-[(2-{5H,6H,7H,8H-imidazo[1,2-a]pyridin-7-ylmethoxy}pyrimidin-5- |
| | yl)methyl]isoquinoline-1,6-diamine |
| 1096 | N4-((6-(((1-methylpiperidin-4-yl)methyl)amino)pyridin-3-yl)methyl)pyridine-2,4-diamine |
| 1102 | 8-methyl-N-((6-(((1-methylpiperidin-4-yl)methyl)amino)pyridin-3-yl)methyl)isoquinolin- |
| | 6-amine |
| 1103 | 8-methoxy-N-((6-(((1-methylpiperidin-4-yl)methyl)amino)pyridin-3-yl)methyl)isoquinolin- |
| | 6-amine |
| 1116 | 5-(((1-(3-chlorophenyl)-1H-pyrazol-4-yl)amino)methyl)-N-((1-methylpiperidin-4- |
| | yl)methyl)pyridin-2-amine |
| 1118 | 4-chloro-6-N-{[6-({3-methyl-1H,4H,5H,6H-cyclopenta[c]pyrazol-6-yl}amino)pyridin-3- |
| | yl]methyl}isoquinoline-1,6-diamine |
| 1119 | N5-((6-((3-methyl-1,4,5,6-tetrahydrocyclopenta[c]pyrazol-6-yl)amino)pyridin-3- |
| | yl)methyl)isoquinoline-1,5-diamine |
| 1128 | 5-N-[(6-{5H,6H,8H-imidazo[1,2-a]piperazin-7-yl}pyridin-3-yl)methyl]isoquinoline-1,5- |
| | diamine |
| 1129 | 4-chloro-6-N-[(6-{5H,6H,8H-imidazo[1,2-a]pyrazin-7-yl}pyridin-3-yl)methyl]isoquinoline- |
| | 1,6-diamine |
| 1130 | (R)-2-(1-(5-(((1-aminoisoquinolin-5-yl)amino)methyl)pyridin-2- |
| | yl)pyrrolidin-3-yl)propan-2-ol |
| 1131 | (S)-2-(1-(5-(((1-aminoisoquinolin-5-yl)amino)methyl)pyridin-2- |
| | yl)pyrrolidin-3-yl)propan-2-ol |
| 1133 | (7-(5-(((1-aminoisoquinolin-5-yl)amino)methyl)pyridin-2-yl)-5,6,7,8- |
| | tetrahydroimidazo[1,2-a]pyrazin-5-yl)methanol |
| 1135 | 2-(7-(5-(((1-aminoisoquinolin-5-yl)amino)methyl)pyridin-2-yl)-5,6,7,8- |
| | tetrahydroimidazo[1,2-a]pyrazin-5-yl)propan-2-ol |
| 1137 | (7-(5-(((1-aminoisoquinolin-5-yl)amino)methyl)pyridin-2-yl)-5,6,7,8- |
| | tetrahydroimidazo[1,2-a]pyrazin-6-yl)methanol |
| 1140 | (7-(5-(((1-aminoisoquinolin-5-yl)amino)methyl)pyridin-2-yl)-5,6,7,8- |
| | tetrahydroimidazo[1,2-a]pyrazin-8-yl)methanol |
| 1150 | N5-((6-(8-methyl-5,6-dihydroimidazo[1,2-a]pyrazin-7(8H)-yl)pyridin-3- |
| | yl)methyl)isoquinoline-1,5-diamine |
| 1156 | N5-((6-(6,7-dihydropyrazolo[1,5-a]pyrazin-5(4H)-yl)pyridin-3-yl)methyl)isoquinoline-1,5- |
| | diamine |
| 1157 | 4-chloro-N6-((6-(6,7-dihydropyrazolo[1,5-a]pyrazin-5(4H)-yl)pyridin-3- |
| | yl)methyl)isoquinoline-1,6-diamine |
| 1163 | 2-(7-(5-(((1-aminoisoquinolin-5-yl)amino)methyl)pyridin-2-yl)-5,6,7,8- |
| | tetrahydroimidazo[1,2-a]pyrazin-2-yl)propan-2-ol |
| 1167 | 2-(7-(5-(((1-aminoisoquinolin-5-yl)amino)methyl)pyridin-2-yl)-5,6,7,8- |
| | tetrahydroimidazo[1,2-a]pyrazin-3-yl)propan-2-ol |
| 1175 | 6-N-[(6-{[(1R,5S,6S)-3-methyl-3-azabicyclo[3.1.0]hexan-6-yl]methoxy}pyridin-3- |
| | yl)methyl]isoquinoline-1,6-diamine |
| 1176 | 2-[(3S)-1-(5-{[(1-amino-4-chloroisoquinolin-6-yl)amino]methyl}pyridin-2-yl)pyrrolidin-3- |
| | yl]propan-2-ol |
| 1177 | 6-methoxy-N-((6-(((1-methylpiperidin-4-yl)methyl)amino)pyridin-3-yl)methyl)quinolin- |
| | 3-amine |
| 1178 | 5-(((8-methoxynaphthalen-2-yl)amino)methyl)-N-((1-methylpiperidin-4- |
| | yl)methyl)pyridin-2-amine |
| 1180 | 5-(((6-methoxynaphthalen-2-yl)amino)methyl)-N-((1-methylpiperidin-4- |
| | yl)methyl)pyridin-2-amine |
| 1181 | N5-((6-((2-(1-(difluoromethyl)-1H-imidazol-2-yl)ethyl)amino)pyridin-3- |
| | yl)methyl)isoquinoline-1,5-diamine |
| 1182 | 4-chloro-N6-((6-((2-(1-(difluoromethyl)-1H-imidazol-2-yl)ethyl)amino)pyridin-3- |
| | yl)methyl)isoquinoline-1,6-diamine |
| 1183 | N-((6-(((1-methylpiperidin-4-yl)methyl)amino)pyridin-3-yl)methyl)quinolin-3-amine |
| 1184 | N-((6-(((1-methylpiperidin-4-yl)methyl)amino)pyridin-3-yl)methyl)quinolin-7-amine |
| 1185 | 4-chloro-N6-((6-((1-(difluoromethyl)-1H-imidazol-2-yl)(methyl)amino)pyridin-3- |
| | yl)methyl)isoquinoline-1,6-diamine |
| 1186 | N3,N3-dimethyl-N6-((6-(((1-methylpiperidin-4-yl)methyl)amino)pyridin-3- |
| | yl)methyl)pyridazine-3,6-diamine |
| 1187 | N-((6-(((1-methylpiperidin-4-yl)methyl)amino)pyridin-3-yl)methyl)imidazo[1,2- |
| | b]pyridazin-3-amine |
| 1188 | 4-chloro-N-((6-(((1-methylpiperidin-4-yl)methyl)amino)pyridin-3-yl)methyl)isoquinolin- |
| | 6-amine |
| 1189 | N-((6-(((1-methylpiperidin-4-yl)methyl)amino)pyridin-3-yl)methyl)pyrido[2,3-b]pyrazin- |
| | 7-amine |
| 1190 | N-((6-(((1-methylpiperidin-4-yl)methyl)amino)pyridin-3-yl)methyl)imidazo[1,2-a]pyridin- |
| | 8-amine |
| 1191 | 4-fluoro-N-((6-(((1-methylpiperidin-4-yl)methyl)amino)pyridin-3-yl)methyl)-1H- |
| | benzo[d]imidazol-5-amine |
| 1192 | 1-methyl-N-((6-(((1-methylpiperidin-4-yl)methyl)amino)pyridin-3-yl)methyl)-1H- |
| | benzo[d]imidazol-2-amine |
| 1193 | 4-chloro-N6-((6-(((1-methylpiperidin-4-yl)methyl)amino)pyridin-3- |
| | yl)methyl)isoquinoline-1,6-diamine |
| 1194 | 4-fluoro-N5-((6-(((1-methylpiperidin-4-yl)methyl)amino)pyridin-3- |
| | yl)methyl)isoquinoline-1,5-diamine |
| 1195 | 4-fluoro-N6-((6-(((1-methylpiperidin-4-yl)methyl)amino)pyridin-3- |
| | yl)methyl)isoquinoline-1,6-diamine |
| 1196 | N6-((6-(((1-methylpiperidin-4-yl)methyl)amino)pyridin-3-yl)methyl)-2,7-naphthyridine- |
| | 1,6-diamine |
| 1197 | N-((6-(((1-methylpiperidin-4-yl)methyl)amino)pyridin-3-yl)methyl)-1H-pyrrolo[2,3- |
| | b]pyridin-4-amine |
| 1198 | N-((6-(((1-methylpiperidin-4-yl)methyl)amino)pyridin-3-yl)methyl)-4-(piperidin-1- |
| | yl)isoquinolin-6-amine |
| 1199 | 7-(((6-(((1-methylpiperidin-4-yl)methyl)amino)pyridin-3-yl)methyl)amino)pyrrolo[1,2- |
| | a]pyrazin-1(2H)-one |
| 1200 | 4-methoxy-N-((6-(((1-methylpiperidin-4-yl)methyl)amino)pyridin-3- |
| | yl)methyl)benzo[d]thiazol-2-amine |
| 1201 | 6-(((6-(((1-methylpiperidin-4-yl)methyl)amino)pyridin-3-yl)methyl)amino)isoquinolin- |
| | 3(2H)-one |
| 1202 | 2-methyl-N-((6-(((1-methylpiperidin-4-yl)methyl)amino)pyridin-3-yl)methyl)-1H- |
| | pyrrolo[2,3-b]pyridin-4-amine |
| 1203 | 6-(((6-(((1-methylpiperidin-4-yl)methyl)amino)pyridin-3-yl)methyl)amino)isoquinoline- |
| | 8-carboxylic acid |
| 1204 | 3-chloro-5-(((6-(((1-methylpiperidin-4-yl)methyl)amino)pyridin-3-yl)methyl)amino)-1H- |
| | indole-7-carboxylic acid |
| 1205 | 5-methyl-N-((6-(((1-methylpiperidin-4-yl)methyl)amino)pyridin-3-yl)methyl)-1H- |
| | pyrrolo[2,3-b]pyridin-4-amine |
| 1206 | 5-fluoro-N-((6-(((1-methylpiperidin-4-yl)methyl)amino)pyridin-3-yl)methyl)-1H- |
| | pyrrolo[2,3-b]pyridin-4-amine |
| 1207 | 8-fluoro-N-((6-(((1-methylpiperidin-4-yl)methyl)amino)pyridin-3-yl)methyl)isoquinolin- |
| | 6-amine |
| 1208 | 8-chloro-N-((6-(((1-methylpiperidin-4-yl)methyl)amino)pyridin-3-yl)methyl)isoquinolin- |
| | 6-amine |
| 1209 | 5-chloro-N-((6-(((1-methylpiperidin-4-yl)methyl)amino)pyridin-3-yl)methyl)isoquinolin- |
| | 6-amine |
| 1210 | N-methyl-6-(((6-(((1-methylpiperidin-4-yl)methyl)amino)pyridin-3- |
| | yl)methyl)amino)isoquinoline-8-carboxamide |
| 1211 | (6-(((6-(((1-methylpiperidin-4-yl)methyl)amino)pyridin-3-yl)methyl)amino)isoquinolin-8- |
| | yl)methanol |
| 1212 | 5-(((6-(((1-methylpiperidin-4-yl)methyl)amino)pyridin-3-yl)methyl)amino)-1H- |
| | pyrrolo[2,3-b]pyridine-3-carbonitrile |
| 1213 | N-((6-(((1-methylpiperidin-4-yl)methyl)amino)pyridin-3-yl)methyl)-1H-pyrrolo[2,3- |
| | c]pyridin-4-amine |
| 1214 | 8-fluoro-N-((6-(((1-methylpiperidin-4-yl)methyl)amino)pyridin-3-yl)methyl)isoquinolin- |
| | 5-amine |
| 1215 | 6-methyl-N-((6-(((1-methylpiperidin-4-yl)methyl)amino)pyridin-3-yl)methyl)-1H-indol-4- |
| | amine |
| 1216 | 1,2-dimethyl-N-((6-(((1-methylpiperidin-4-yl)methyl)amino)pyridin-3-yl)methyl)-1H- |
| | pyrrolo[2,3-b]pyridin-4-amine |
| 1217 | 7-methoxy-N-((6-(((1-methylpiperidin-4-yl)methyl)amino)pyridin-3-yl)methyl)quinolin- |
| | 4-amine |
| 1218 | N-((6-(((1-methylpiperidin-4-yl)methyl)amino)pyridin-3-yl)methyl)-1H-pyrazolo[3,4- |
| | b]pyridin-4-amine |
| 1219 | N2-methyl-N4-((6-(((1-methylpiperidin-4-yl)methyl)amino)pyridin-3-yl)methyl)pyridine- |
| | 2,4-diamine |
| 1220 | 3-chloro-N-methyl-5-(((6-(((1-methylpiperidin-4-yl)methyl)amino)pyridin-3- |
| | yl)methyl)amino)-1H-indole-7-carboxamide |
| 1221 | 4-(((6-(((1-methylpiperidin-4-yl)methyl)amino)pyridin-3-yl)methyl)amino)-1H- |
| | pyrrolo[2,3-b]pyridine-3-carbonitrile |
| 1222 | N-((6-(((1-methylpiperidin-4-yl)methyl)amino)pyridin-3-yl)methyl)imidazo[1,2-a]pyridin- |
| | 7-amine |
| 1223 | N-((6-(((1-methylpiperidin-4-yl)methyl)amino)pyridin-3-yl)methyl)-1H-pyrrolo[3,2- |
| | c]pyridin-4-amine |
| 1224 | 5-fluoro-N-((6-(((1-methylpiperidin-4-yl)methyl)amino)pyridin-3-yl)methyl)isoquinolin- |
| | 6-amine |
| 1225 | 1-methyl-N-((6-(((1-methylpiperidin-4-yl)methyl)amino)pyridin-3-yl)methyl)-1H- |
| | pyrrolo[2,3-b]pyridin-4-amine |
| 1226 | 3-methyl-N-((6-(((1-methylpiperidin-4-yl)methyl)amino)pyridin-3-yl)methyl)-1H- |
| | pyrrolo[2,3-b]pyridin-4-amine |
| 1227 | N-((6-(((1-methylpiperidin-4-yl)methyl)amino)pyridin-3-yl)methyl)-7H-pyrrolo[2,3- |
| | d]pyrimidin-4-amine |
| 1228 | 2-methyl-N-((6-(((1-methylpiperidin-4-yl)methyl)amino)pyridin-3-yl)methyl)-1H-indol-4- |
| | amine |
| 1229 | N-((6-(((1-methylpiperidin-4-yl)methyl)amino)pyridin-3-yl)methyl)-3H-imidazo[4,5- |
| | b]pyridin-7-amine |
| 1230 | N-((6-(((1-methylpiperidin-4-yl)methyl)amino)pyridin-3-yl)methyl)-1H-indol-4-amine |
| 1231 | 2-isopropyl-N-((6-(((1-methylpiperidin-4-yl)methyl)amino)pyridin-3-yl)methyl)-1H- |
| | pyrrolo[2,3-b]pyridin-4-amine |
| 1232 | 2-methyl-N-((6-(((1-methylpiperidin-4-yl)methyl)amino)pyridin-3-yl)methyl)-1H- |
| | pyrrolo[2,3-c]pyridin-4-amine |
| 1233 | 6-methoxy-N-((6-(((1-methylpiperidin-4-yl)methyl)amino)pyridin-3-yl)methyl)-1H- |
| | pyrrolo[2,3-b]pyridin-4-amine |
| 1234 | 3-methyl-N-((6-(((1-methylpiperidin-4-yl)methyl)amino)pyridin-3-yl)methyl)-1H- |
| | pyrrolo[2,3-b]pyridin-5-amine |
| 1235 | 2-methyl-N-((6-(((1-methylpiperidin-4-yl)methyl)amino)pyridin-3-yl)methyl)-1H- |
| | pyrrolo[2,3-b]pyridin-5-amine |
| 1236 | 4-(((6-(((1-methylpiperidin-4-yl)methyl)amino)pyridin-3-yl)methyl)amino)-1,3-dihydro- |
| | 2H-pyrrolo[2,3-b]pyridin-2-one |
| 1237 | N-((6-(((1-methylpiperidin-4-yl)methyl)amino)pyridin-3-yl)methyl)-6-(trifluoromethyl)- |
| | 1H-indol-4-amine |
| 1238 | 1-methyl-N-((6-(((1-methylpiperidin-4-yl)methyl)amino)pyridin-3-yl)methyl)-6- |
| | (trifluoromethyl)-1H-indol-4-amine |
| 1239 | 2-ethyl-N-((6-(((1-methylpiperidin-4-yl)methyl)amino)pyridin-3-yl)methyl)-1H- |
| | pyrrolo[2,3-b]pyridin-4-amine |
| 1240 | 7-methyl-N-((6-(((1-methylpiperidin-4-yl)methyl)amino)pyridin-3-yl)methyl)-1H-indol-4- |
| | amine |
| 1241 | 6-chloro-N-((6-(((1-methylpiperidin-4-yl)methyl)amino)pyridin-3-yl)methyl)-1H-indol-4- |
| | amine |
| 1242 | 7-methoxy-N-((6-(((1-methylpiperidin-4-yl)methyl)amino)pyridin-3-yl)methyl)-1H-indol- |
| | 4-amine |
| 1243 | 2-chloro-N-((6-(((1-methylpiperidin-4-yl)methyl)amino)pyridin-3-yl)methyl)-1H- |
| | pyrrolo [2,3-b]pyridin-4-amine |
| 1244 | 7-methyl-N-((6-(((1-methylpiperidin-4-yl)methyl)amino)pyridin-3-yl)methyl)-1H- |
| | pyrrolo[2,3-c]pyridin-4-amine |
| 1245 | 8-fluoro-N6-((6-(((1-methylpiperidin-4-yl)methyl)amino)pyridin-3- |
| | yl)methyl)isoquinoline-1,6-diamine |
| 1246 | 6-methyl-N-((6-(((1-methylpiperidin-4-yl)methyl)amino)pyridin-3-yl)methyl)-1H- |
| | pyrrolo[2,3-b]pyridin-4-amine |
| 1247 | methyl 4-(((6-(((1-methylpiperidin-4-yl)methyl)amino)pyridin-3-yl)methyl)amino)-1H- |
| | pyrrolo[2,3-b]pyridine-2-carboxylate |
| 1248 | methyl 4-(((6-(((1-methylpiperidin-4-yl)methyl)amino)pyridin-3-yl)methyl)amino)-1H- |
| | pyrrolo[2,3-b]pyridine-3-carboxylate |
| 1249 | 2-methyl-N-((6-((1-methylpiperidin-4-yl)methoxy)pyridin-3-yl)methyl)-1H-pyrrolo[2,3- |
| | b]pyridin-4-amine |
| 1250 | N5-((6-(3-methoxy-5,6-dihydro-[1,2,4]triazolo[4,3-a]pyrazin-7(8H)-yl)pyridin-3- |
| | yl)methyl)isoquinoline-1,5-diamine |
| 1251 | 2-chloro-N-((6-(((1-methylpiperidin-4-yl)methyl)amino)pyridin-3-yl)methyl)-1H- |
| | pyrrolo[2,3-c]pyridin-4-amine |
| 1252 | 4-(((6-(((1-methylpiperidin-4-yl)methyl)amino)pyridin-3-yl)methyl)amino)-1H- |
| | pyrrolo[2,3-b]pyridine-2-carboxamide |
| 1253 | 4-(((6-(((1-methylpiperidin-4-yl)methyl)amino)pyridin-3-yl)methyl)amino)-1H- |
| | pyrrolo [2,3-b]pyridine-2-carboxylic acid |
| 1254 | 6-methyl-N-((6-(((1-methylpiperidin-4-yl)methyl)amino)pyridin-3-yl)methyl)-7H- |
| | pyrrolo[2,3-d]pyrimidin-4-amine |
| 1255 | 2-chloro-5-methyl-N-((6-(((1-methylpiperidin-4-yl)methyl)amino)pyridin-3-yl)methyl)- |
| | 1H-pyrrolo[2,3-b]pyridin-4-amine |
| 1256 | 4-(methyl(6-(((1-methylpiperidin-4-yl)methyl)amino)pyridin-3-yl)amino)-1H-indole-6- |
| | carbonitrile |
| 1257 | 2,2-dimethyl-N-((6-(((1-methylpiperidin-4-yl)methyl)amino)pyridin-3-yl)methyl)-2,3- |
| | dihydro-1H-pyrrolo[2,3-b]pyridin-4-amine |
| 1258 | N-((1-methylpiperidin-4-yl)methyl)-5-((pyridin-4-ylamino)methyl)pyridin-2-amine |
| 1259 | 4-(methyl(6-(((1-methylpiperidin-4-yl)methyl)amino)pyridin-3-yl)amino)-1H-indole-6- |
| | carbonitrile |
| 1260 | 4-(3,3-difluoropyrrolidin-1-yl)-N-((6-(((1-methylpiperidin-4-yl)methyl)amino)pyridin-3- |
| | yl)methyl)isoquinolin-6-amine |
| 1261 | 4-(((6-(((1-methylpiperidin-4-yl)methyl)amino)pyridin-3-yl)methyl)amino)-1H- |
| | pyrrolo[2,3-b]pyridine-6-carbonitrile |
| 1262 | 2-chloro-N-((6-(((1-methylpiperidin-4-yl)methyl)amino)pyridin-3-yl)methyl)-6- |
| | (trifluoromethyl)-1H-indol-4-amine |
| 1263 | 4-(((6-(((1-methylpiperidin-4-yl)methyl)amino)pyridin-3-yl)methyl)amino)-1H- |
| | pyrrolo [2,3-b]pyridine-6-carboxylic acid |
| 1264 | 4-(((6-(((1-methylpiperidin-4-yl)methyl)amino)pyridin-3-yl)methyl)amino)-1H- |
| | pyrrolo[2,3-b]pyridine-6-carboxamide |
| 1265 | N5-((6-(3-(2,2-difluoroethyl)pyrrolidin-1-yl)pyridin-3-yl)methyl)isoquinoline-1,5-diamine |
| 1266 | 4-chloro-N6-((6-(3-(2,2-difluoroethyl)pyrrolidin-1-yl)pyridin-3-yl)methyl)isoquinoline- |
| | 1,6-diamine |
| 1267 | 5-(5-(((1-amino-4-chloroisoquinolin-6-yl)amino)methyl)pyridin-2-yl)-4,5,6,7- |
| | tetrahydropyrazolo[1,5-a]pyrazin-3-ol |
| 1268 | (5-(5-(((1-aminoisoquinolin-5-yl)amino)methyl)pyridin-2-yl)-4,5,6,7- |
| | tetrahydropyrazolo[1,5-a]pyrazin-4-yl)methanol |
| 1269 | (5-(5-(((1-aminoisoquinolin-5-yl)amino)methyl)pyridin-2-yl)-4,5,6,7- |
| | tetrahydropyrazolo[1,5-a]pyrazin-3-yl)methanol |
| 1270 | 1-(5-(((1-amino-4-chloroisoquinolin-6-yl)amino)methyl)pyridin-2-yl)-6′,7′- |
| | dihydrospiro[azetidine-3,5′-pyrrolo[1,2-a]imidazol]-7′-ol |
| 1271 | N5-((6-(5,6,8,9-tetrahydro-7H-imidazo[1,2-d][1,4]diazepin-7-yl)pyridin-3- |
| | yl)methyl)isoquinoline-1,5-diamine |
| 1272 | 4-chloro-N6-((6-(5,6,8,9-tetrahydro-7H-imidazo[1,2-d][1,4]diazepin-7-yl)pyridin-3- |
| | yl)methyl)isoquinoline-1,6-diamine |
| 1273 | (5-(5-(((1-amino-4-chloroisoquinolin-6-yl)amino)methyl)pyridin-2-yl)-4,5,6,7- |
| | tetrahydropyrazolo[1,5-a]pyrazin-4-yl)methanol |
| 1274 | N5-((6-(3-(difluoromethyl)-5,6-dihydro-[1,2,4]triazolo[4,3-a]pyrazin-7(8H)-yl)pyridin-3- |
| | yl)methyl)isoquinoline-1,5-diamine |
| 1275 | N5-((6-(3-(trifluoromethyl)-5,6-dihydro-[1,2,4]triazolo[4,3-a]pyrazin-7(8H)-yl)pyridin-3- |
| | yl)methyl)isoquinoline-1,5-diamine |
| 1276 | N5-((6-(5,6-dihydro-[1,2,4]triazolo[4,3-a]pyrazin-7(8H)-yl)pyridin-3- |
| | yl)methyl)isoquinoline-1,5-diamine |
| 1277 | 2-chloro-N-((6-(3-(difluoromethyl)-5,6-dihydro-[1,2,4]triazolo[4,3-a]pyrazin-7(8H)- |
| | yl)pyridin-3-yl)methyl)-1H-pyrrolo[2,3-b]pyridin-4-amine |
| 1278 | N-((6-(3-(difluoromethyl)-5,6-dihydro-[1,2,4]triazolo[4,3-a]pyrazin-7(8H)-yl)pyridin-3- |
| | yl)methyl)-2-methyl-1H-pyrrolo[2,3-b]pyridin-4-amine |
| 1279 | N5-((6-(3-methyl-5,6-dihydro-[1,2,4]triazolo[4,3-a]pyrazin-7(8H)-yl)pyridin-3- |
| | yl)methyl)isoquinoline-1,5-diamine |
| 1280 | 2-chloro-N-((6-(3-(trifluoromethyl)-5,6-dihydro-[1,2,4]triazolo[4,3-a]pyrazin-7(8H)- |
| | yl)pyridin-3-yl)methyl)-1H-pyrrolo[2,3-b]pyridin-4-amine |
| 1281 | N4-((6-(3-(difluoromethyl)-5,6-dihydro-[1,2,4]triazolo[4,3-a]pyrazin-7(8H)-yl)pyridin-3- |
| | yl)methyl)-N2-methylpyridine-2,4-diamine |
| 1282 | N5-((4-methyl-6-(3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1283 | N-((6-(3-(trifluoromethyl)-5,6-dihydro-[1,2,4]triazolo[4,3-a]pyrazin-7(8H)-yl)pyridin-3- |
| | yl)methyl)isoquinolin-6-amine |
| 1284 | N5-((2-methyl-6-(3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1285 | N5-((6-(8-methyl-3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1286 | N5-((6-(1-methyl-1,4,6,7-tetrahydro-5H-pyrazolo[4,3-c]pyridin-5-yl)pyridin-3- |
| | yl)methyl)isoquinoline-1,5-diamine |
| 1287 | N5-((6-(5-methyl-3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1288 | N-((6-(3-(trifluoromethyl)-5,6-dihydro-[1,2,4]triazolo[4,3-a]pyrazin-7(8H)-yl)pyridin-3- |
| | yl)methyl)imidazo[1,2-a]pyridin-7-amine |
| 1289 | 3-chloro-N-((6-(3-(trifluoromethyl)-5,6-dihydro-[1,2,4]triazolo[4,3-a]pyrazin-7(8H)- |
| | yl)pyridin-3-yl)methyl)-1H-pyrrolo[2,3-b]pyridin-5-amine |
| 1290 | 2-isopropoxy-N-((6-(3-(trifluoromethyl)-5,6-dihydro-[1,2,4]triazolo[4,3-a]pyrazin-7(8H)- |
| | yl)pyridin-3-yl)methyl)pyridin-4-amine |
| 1291 | 2-(1-(5-(((1-aminoisoquinolin-5-yl)amino)methyl)pyridin-2-yl)-1H-1,2,3-triazol-4- |
| | yl)propan-2-ol |
| 1292 | 2-chloro-4-(methyl(6-(3-(trifluoromethyl)-5,6-dihydro-[1,2,4]triazolo[4,3-a]pyrazin- |
| | 7(8H)-yl)pyridin-3-yl)amino)-1H-pyrrolo[2,3-b]pyridine-6-carbonitrile |
| 1293 | 2-chloro-6-methyl-N-((6-(3-(trifluoromethyl)-5,6-dihydro-[1,2,4]triazolo[4,3-a]pyrazin- |
| | 7(8H)-yl)pyridin-3-yl)methyl)-1H-pyrrolo[2,3-b]pyridin-4-amine |
| 1294 | (R*)-N5-((6-(8-methyl-3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 1295 | (S*)-N5-((6-(8-methyl-3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 1296 | (R*)-N5-((6-(5-methyl-3-(trifluoromethyl)-5,6-dihydro-[1,2,4]triazolo[4,3-a]pyrazin- |
| | 7(8H)-yl)468yridine-3-yl)methyl)isoquinoline-1,5-diamine |
| 1297 | (S*)-N5-((6-(5-methyl-3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 1298 | N5-((2-(trifluoromethyl)-6-(3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 1299 | N5-((4-chloro-6-(3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1300 | N5-(4-(3-(trifluoromethyl)-5,6-dihydro-[1,2,4]triazolo[4,3-a]pyrazin-7(8H)- |
| | yl)benzyl)isoquinoline-1,5-diamine |
| 1301 | N5-((4-(trifluoromethyl)-6-(3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 1302 | N5-((5-methyl-6-(3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1303 | N5-((4-methyl-6-(8-methyl-3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 1304 | (R*)-N5-((4-methyl-6-(8-methyl-3-(trifluoromethyl)-5,6-dihydro-[1,2,4]triazolo[4,3- |
| | a]pyrazin-7(8H)-yl)pyridin-3-yl)methyl)isoquinoline-1,5-diamine |
| 1305 | (S*)-N5-((4-methyl-6-(8-methyl-3-(trifluoromethyl)-5,6-dihydro-[1,2,4]triazolo[4,3- |
| | a]pyrazin-7(8H)-yl)pyridin-3-yl)methyl)isoquinoline-1,5-diamine |
| 1306 | N5-((5-(trifluoromethyl)-6-(3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 1307 | N5-((4-cyclopropyl-6-(3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 1308 | N5-((4-isopropyl-6-(3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1309 | N5-((4-ethyl-6-(3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1310 | N5-((6-(3-methyl-5,6,8,9-tetrahydro-7H-[1,2,4]triazolo[4,3-d][1,4]diazepin-7-yl)pyridin- |
| | 3-yl)methyl)isoquinoline-1,5-diamine |
| 1311 | N5-((6-(6-methyl-3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1312 | 5-(5-(((1-aminoisoquinolin-5-yl)amino)methyl)pyridin-2-yl)-4,5,6,7- |
| | tetrahydropyrazolo[1,5-a]pyrazin-3-ol |
| 1313 | 2-chloro-N-((4-methyl-6-(3-(trifluoromethyl)-5,6-dihydro-[1,2,4]triazolo[4,3-a]pyrazin- |
| | 7(8H)-yl)pyridin-3-yl)methyl)-1H-pyrrolo[2,3-b]pyridin-4-amine |
| 1314 | 2-chloro-N-((4-methyl-6-(8-methyl-3-(trifluoromethyl)-5,6-dihydro-[1,2,4]triazolo[4,3- |
| | a]pyrazin-7(8H)-yl)pyridin-3-yl)methyl)-1H-pyrrolo[2,3-b]pyridin-4-amine |
| 1315 | (R*)-2-chloro-N-((4-methyl-6-(8-methyl-3-(trifluoromethyl)-5,6-dihydro- |
| | [1,2,4]triazolo[4,3-a]pyrazin-7(8H)-yl)pyridin-3-yl)methyl)-1H-pyrrolo[2,3-b]pyridin-4- |
| | amine |
| 1316 | (S*)-2-chloro-N-((4-methyl-6-(8-methyl-3-(trifluoromethyl)-5,6-dihydro- |
| | [1,2,4]triazolo[4,3-a]pyrazin-7(8H)-yl)pyridin-3-yl)methyl)-1H-pyrrolo[2,3-b]pyridin-4- |
| | amine |
| 1317 | N5-((6-(3-(trifluoromethyl)-5,6-dihydro-[1,2,4]triazolo[4,3-a]pyrazin-7(8H)-yl)pyridin-3- |
| | yl)methyl)-2,7-naphthyridine-1,5-diamine |
| 1318 | N1-((6-(3-(trifluoromethyl)-5,6-dihydro-[1,2,4]triazolo[4,3-a]pyrazin-7(8H)-yl)pyridin-3- |
| | yl)methyl)-2,6-naphthyridine-1,5-diamine |
| 1319 | N5-((6-(8-(methoxymethyl)-3-(trifluoromethyl)-5,6-dihydro-[1,2,4]triazolo[4,3- |
| | a]pyrazin-7(8H)-yl)-4-methylpyridin-3-yl)methyl)isoquinoline-1,5-diamine |
| 1320 | (R*)-N5-((6-(8-ethyl-3-(trifluoromethyl)-5,6-dihydro-[1,2,4]triazolo[4,3-a]pyrazin-7(8H)- |
| | yl)-4-methylpyridin-3-yl)methyl)isoquinoline-1,5-diamine |
| 1321 | (S*)-N5-((6-(8-ethyl-3-(trifluoromethyl)-5,6-dihydro-[1,2,4]triazolo[4,3-a]pyrazin-7(8H)- |
| | yl)-4-methylpyridin-3-yl)methyl)isoquinoline-1,5-diamine |
| 1322 | N-((4-methyl-6-(3-(trifluoromethyl)-5,6-dihydro-[1,2,4]triazolo[4,3-a]pyrazin-7(8H)- |
| | yl)pyridin-3-yl)methyl)imidazo[1,2-a]pyridin-6-amine |
| 1323 | N4-((4-methyl-6-(3-(trifluoromethyl)-5,6-dihydro-[1,2,4]triazolo[4,3-a]pyrazin-7(8H)- |
| | yl)pyridin-3-yl)methyl)pyridine-2,4-diamine |
| 1324 | 6-(methyl(6-((1-methylpiperidin-4-yl)methoxy)pyridin-3-yl)amino)isoquinoline-1- |
| | carbonitrile |
| 1325 | 5-chloro-N-((6-((1-methylpiperidin-4-yl)methoxy)pyridin-3-yl)methyl)naphthalen-2- |
| | amine |
| 1326 | 1-methyl-N-((6-((1-methylpiperidin-4-yl)methoxy)pyridin-3-yl)methyl)isoquinolin-6- |
| | amine |
| 1327 | N-((6-((1-methylpiperidin-4-yl)methoxy)pyridin-3-yl)methyl)-1,6-naphthyridin-2-amine |
| 1328 | methyl 6-(methyl(6-((1-methylpiperidin-4-yl)methoxy)pyridin-3-yl)amino)isoquinoline- |
| | 4-carboxylate |
| 1329 | 6-(methyl(6-((1-methylpiperidin-4-yl)methoxy)pyridin-3-yl)amino)isoquinoline-4- |
| | carboxylic acid |
| 1330 | 6-(methyl(6-((1-methylpiperidin-4-yl)methoxy)pyridin-3-yl)amino)isoquinoline-4- |
| | carboxamide |
| 1331 | N5-((6-(8-ethyl-3-(trifluoromethyl)-5,6-dihydro-[1,2,4]triazolo[4,3-a]pyrazin-7(8H)-yl)-4- |
| | methylpyridin-3-yl)methyl)isoquinoline-1,5-diamine |
| 1332 | N5-methyl-N5-((6-(3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1333 | N6-((4-methyl-6-(3-(trifluoromethyl)-5,6-dihydro-[1,2,4]triazolo[4,3-a]pyrazin-7(8H)- |
| | yl)pyridin-3-yl)methyl)isoquinoline-1,6-diamine |
| 1334 | 4-chloro-N6-((4-methyl-6-(3-(trifluoromethyl)-5,6-dihydro-[1,2,4]triazolo[4,3-a]pyrazin- |
| | 7(8H)-yl)pyridin-3-yl)methyl)isoquinoline-1,6-diamine |
| 1335 | (5-(((1-aminoisoquinolin-5-yl)amino)methyl)-2-(3-(trifluoromethyl)-5,6-dihydro- |
| | [1,2,4]triazolo[4,3-a]pyrazin-7(8H)-yl)pyridin-3-yl)methanol |
| 1336 | N5-((2-ethyl-6-(3-(trifluoromethyl)-5,6-dihydro-[1,2,4]triazolo[4,3-a]pyrazin-7(8H)- |
| | yl)pyridin-3-yl)methyl)isoquinoline-1,5-diamine |
| 1337 | N-(1-aminoisoquinolin-5-yl)-6-(3-(trifluoromethyl)-5,6-dihydro-[1,2,4]triazolo[4,3- |
| | a]pyrazin-7(8H)-yl)pyridine-3-sulfonamide |
| 1338 | 4-(methyl(6-(3-(trifluoromethyl)-5,6-dihydro-[1,2,4]triazolo[4,3-a]pyrazin-7(8H)- |
| | yl)pyridin-3-yl)amino)-1H-indole-6-carbonitrile |
| 1339 | N-((4-methyl-6-(3-(trifluoromethyl)-5,6-dihydro-[1,2,4]triazolo[4,3-a]pyrazin-7(8H)- |
| | yl)pyridin-3-yl)methyl)-2,3-dihydro-1H-pyrrolo[2,3-b]pyridin-4-amine |
| 1341 | 2-methyl-N-((4-methyl-6-(3-(trifluoromethyl)-5,6-dihydro-[1,2,4]triazolo[4,3-a]pyrazin- |
| | 7(8H)-yl)pyridin-3-yl)methyl)-1H-pyrrolo[2,3-c]pyridin-4-amine |
| 1342 | (R)-N5-((4-methyl-6-(6-methyl-3-(trifluoromethyl)-5,6-dihydro-[1,2,4]triazolo[4,3- |
| | a]pyrazin-7(8H)-yl)pyridin-3-yl)methyl)isoquinoline-1,5-diamine |
| 1343 | (S*)-N5-((6-(8-(methoxymethyl)-3-(trifluoromethyl)-5,6-dihydro-[1,2,4]triazolo[4,3- |
| | a]pyrazin-7(8H)-yl)-4-methylpyridin-3-yl)methyl)isoquinoline-1,5-diamine |
| 1344 | (R*)-N5-((6-(8-(methoxymethyl)-3-(trifluoromethyl)-5,6-dihydro-[1,2,4]triazolo[4,3- |
| | a]pyrazin-7(8H)-yl)-4-methylpyridin-3-yl)methyl)isoquinoline-1,5-diamine |
| 1345 | (R*)-N5-((3-methyl-5-(8-methyl-3-(trifluoromethyl)-5,6-dihydro-[1,2,4]triazolo[4,3- |
| | a]pyrazin-7(8H)-yl)pyridin-2-yl)methyl)isoquinoline-1,5-diamine |
| 1346 | (S*)-N5-((3-methyl-5-(8-methyl-3-(trifluoromethyl)-5,6-dihydro-[1,2,4]triazolo[4,3- |
| | a]pyrazin-7(8H)-yl)pyridin-2-yl)methyl)isoquinoline-1,5-diamine |
| 2177 | 6-N-({4-[2-(1-methylpiperidin-4-yl)ethyl]phenyl}methyl)isoquinoline-1,6-diamine |
| 2178 | 5-N-({4-[2-(1-methylpiperidin-4-yl)ethyl]phenyl}methyl)isoquinoline-1,5-diamine |
| 2179 | 2-(4-{[(1-aminoisoquinolin-6-yl)amino]methyl}phenyl)-1-(4-methylpiperazin-1- |
| | yl)ethanone |
| 2180 | 6-N-({4-[2-(4-methylpiperazin-1-yl)ethyl]phenyl}methyl)isoquinoline-1,6-diamine |
| 2181 | 6-N-({2-fluoro-4-[2-(4-methylpiperazin-1-yl)ethyl]phenyl}methyl)isoquinoline-1,6- |
| | diamine |
| 2182 | 5-N-({2-fluoro-4-[2-(4-methylpiperazin-1-yl)ethyl]phenyl}methyl)isoquinoline-1,5- |
| | diamine |
| 2183 | 5-N-({2-fluoro-4-[2-(morpholin-4-yl)ethyl]phenyl}methyl)isoquinoline-1,5-diamine |
| 2184 | 4-chloro-6-N-({2-fluoro-4-[2-(4-methylpiperazin-1-yl)ethyl]phenyl}methyl)isoquinoline- |
| | 1,6-diamine |
| 2185 | 6-N-[(4-{[(1-methylpiperidin-4-yl)oxy]methyl}phenyl)methyl]isoquinoline-1,6-diamine |
| 2186 | 5-N-[(4-{[(1-methylpiperidin-4-yl)oxy]methyl}phenyl)methyl]isoquinoline-1,5-diamine |
| 2187 | 6-N-[(4-{[(1-methylpiperidin-4-yl)oxy]methyl}phenyl)methyl]quinoline-2,6-diamine |
| 2188 | 7-N-[(4-{[(1-methylpiperidin-4-yl)oxy]methyl}phenyl)methyl]isoquinoline-3,7-diamine |
| 2189 | 6-N-[(4-{[(1-methylpiperidin-4-yl)oxy]methyl}phenyl)methyl]isoquinoline-3,6-diamine |
| 2190 | 6-N-methyl-6-N-[(4-{[(1-methylpiperidin-4-yl)oxy]methyl}phenyl)methyl]isoquinoline- |
| | 1,6-diamine |
| 2191 | 6-N-[(2-fluoro-4-{[(1-methylpiperidin-4-yl)oxy]methyl}phenyl)methyl]isoquinoline- |
| | 1,6-diamine |
| 2192 | 6-N-[(2-chloro-4-{[(1-methylpiperidin-4-yl)oxy]methyl}phenyl)methyl]isoquinoline- |
| | 1,6-diamine |
| 2193 | N-[(4-{[(1-methylpiperidin-4-yl)oxy]methyl}phenyl)methyl]isoquinolin-6-amine |
| 2194 | 6-N-[(4-{[(3,3-difluoro-1-methylpiperidin-4-yl)oxy]methyl}phenyl)methyl]isoquinoline- |
| | 1,6-diamine |
| 2195 | 6-N-[(4-{[(1-cyclopropylpiperidin-4-yl)oxy]methyl}phenyl)methyl]isoquinoline-1,6- |
| | diamine |
| 2196 | 6-N-{[4-({[1-(2,2-difluoroethyl)piperidin-4-yl]oxy}methyl)phenyl]methyl}isoquinoline- |
| | 1,6-diamine |
| 2197 | 5-N-[(2-fluoro-4-{[(1-methylpiperidin-4-yl)oxy]methyl}phenyl)methyl]isoquinoline-1,5- |
| | diamine |
| 2198 | N6-(2-fluoro-4-((((4R*,5R*)-2-methyl-2-azabicyclo[2.2.1]heptan-5- |
| | yl)oxy)methyl)benzyl)isoquinoline-1,6-diamine |
| 2199 | N6-(2-fluoro-4-((((4R*,5S*)-2-methyl-2-azabicyclo[2.2.1]heptan-5- |
| | yl)oxy)methyl)benzyl)isoquinoline-1,6-diamine |
| 2200 | 1-(4-((4-(((1-aminoisoquinolin-6-yl)amino)methyl)-3-fluorobenzyl)oxy)piperidin-1- |
| | yl)ethan-1-one |
| 2201 | 5-N-[(4-{[(1-ethylpiperidin-4-yl)oxy]methyl}-2-fluorophenyl)methyl]isoquinoline-1,5- |
| | diamine |
| 2202 | 6-N-[(4-{[(1-ethylpiperidin-4-yl)oxy]methyl}-2-fluorophenyl)methyl]isoquinoline-1,6- |
| | diamine |
| 2203 | 5-N-[(2-chloro-4-{[(1-methylpiperidin-4-yl)oxy]methyl}phenyl)methyl]-5-N- |
| | methylisoquinoline-1,5-diamine |
| 2204 | N-methyl-N-[(4-{[(1-methylpiperidin-4-yl)oxy]methyl}phenyl)methyl]isoquinolin-6- |
| | amine |
| 2205 | 6-N-({2-fluoro-4-[({5-methyl-2-oxa-5-azaspiro[3.5]nonan-8- |
| | yl}oxy)methyl]phenyl}methyl)isoquinoline-1,6-diamine |
| 2206 | 5-N-[[2-fluoro-4-[(2-methyl-2-azaspiro[3.3]heptan-6- |
| | yl)oxymethyl]phenyl]methyl]isoquinoline-1,5-diamine |
| 2207 | N5-(2-fluoro-4-(((1-methyl-1H-imidazol-2-yl)methoxy)methyl)benzyl)isoquinoline-1,5- |
| | diamine |
| 2208 | 5-N-[[2-fluoro-4-[2-(1-methylimidazol-2-yl)ethoxymethyl]phenyl]methyl]isoquinoline- |
| | 1,5-diamine |
| 2209 | 5-[[4-[[(1-amino-5-isoquinolyl)amino]methyl]-3-fluoro-phenyl]methoxy]-1-methyl- |
| | piperidin-2-one |
| 2210 | N-(1-aminoisoquinolin-6-yl)-4-{[(1-methylpiperidin-4- |
| | yl)oxy]methyl}benzenesulfonamide |
| 2211 | N-(4-(((1-aminoisoquinolin-5-yl)amino)methyl)-3-fluorobenzyl)-2-(1-methyl-1H- |
| | imidazol-2-yl)acetamide |
| 2212 | 5-N-[(2-fluoro-4-{2-[(15,4S)-5-isopropyl-2,5-diazabicyclo[2.2.1]heptan-2- |
| | yl]ethyl}phenyl)methyl]isoquinoline-1,5-diamine |
| 2213 | 5-N-[(2-fluoro-4-{2-[(1R,4R)-5-isopropyl-2,5-diazabicyclo[2.2.1]heptan-2- |
| | yl]ethyl}phenyl)methyl]isoquinoline-1,5-diamine |
| 2214 | 4-chloro-6-N-[(2-fluoro-4-{2-[(1R,4R)-5-isopropyl-2,5-diazabicyclo[2.2.1]heptan-2- |
| | yl]ethyl}phenyl)methyl]isoquinoline-1,6-diamine |
| 2215 | 4-chloro-6-N-({2-fluoro-4-[2-(morpholin-4-yl)ethyl]phenyl}methyl)isoquinoline-1,6- |
| | diamine |
| 2216 | 6-N-[(2-fluoro-4-{[(1-isopropylpiperidin-4-yl)oxy]methyl}phenyl)methyl]isoquinoline- |
| | 1,6-diamine |
| 2252 | 6-N-({2-fluoro-4-[({2-methyl-5-thia-2-azaspiro[3.4]octan-7- |
| | yl}oxy)methyl]phenyl}methyl)isoquinoline-1,6-diamine |
| 2253 | 1-(4-(((1-aminoisoquinolin-5-yl)amino)methyl)phenyl)-6′,7′-dihydrospiro[azetidine-3,5′- |
| | pyrrolo[1,2-a]imidazol]-7′-ol |
| 2254 | N5-(2-fluoro-4-((((1R,5S)-9-methyl-3-oxa-9-azabicyclo[3.3.1]nonan-7- |
| | yl)oxy)methyl)benzyl)isoquinoline-1,5-diamine |
| 2255 | N-(4-(((1-methylpiperidin-4-yl)oxy)methyl)benzyl)isoquinolin-7-amine |
| 2256 | N5-(2-fluoro-4-(3-(trifluoromethyl)-5,6-dihydro-[1,2,4]triazolo[4,3-a]pyrazin-7(8H)- |
| | yl)benzyl)isoquinoline-1,5-diamine |
| 2257 | N5-(2-methyl-4-(3-(trifluoromethyl)-5,6-dihydro-[1,2,4]triazolo[4,3-a]pyrazin-7(8H)- |
| | yl)benzyl)isoquinoline-1,5-diamine |
| 3253 | 6-N-({1-[(1-methylpiperidin-4-yl)methyl]pyrazol-4-yl}methyl)isoquinoline-1,6-diamine |
| 3254 | 6-N-({1-[2-(1-methylpiperidin-4-yl)ethyl]pyrazol-4-yl}methyl)isoquinoline-1,6-diamine |
| 3255 | N5-((1-(2-(1-methylpiperidin-4-yl)ethyl)-1H-pyrazol-4-yl)methyl)isoquinoline-1,5- |
| | diamine |
| 4259 | N6-((2-((1-methylpiperidin-4-yl)oxy)pyridin-4-yl)methyl)isoquinoline-1,6-diamine |
| 4260 | 5-N-[[2-[(1-methyl-4-piperidyl)methoxy]-4-pyridyl]methyl]isoquinoline-1,5-diamine |
| 4261 | 6-N-({2-[(1-methylpiperidin-4-yl)methoxy]pyridin-4-yl}methyl)isoquinoline-1,6-diamine |
| 4262 | N-({2-[(1-methylpiperidin-4-yl)methoxy]pyridin-4-yl}methyl)isoquinolin-6-amine |
| 4263 | 7-N-({2-[(1-methylpiperidin-4-yl)methoxy]pyridin-4-yl}methyl)quinazoline-4,7-diamine |
| 4264 | 3-chloro-N-((2-((1-methylpiperidin-4-yl)methoxy)pyridin-4-yl)methyl)-1H-pyrrolo[2,3- |
| | b]pyridin-5-amine |
| 4265 | 6-N-({2-[(1-methylpiperidin-4-yl)methoxy]-6-(trifluoromethyl)pyridin-4- |
| | yl}methyl)isoquinoline-1,6-diamine |
| 4266 | 6-N-[(2-{[2-(trifluoromethyl)-5H,6H,7H,8H-imidazo[1,2-a]pyridin-7-yl]methoxy}pyridin- |
| | 4-yl)methyl]isoquinoline-1,6-diamine |
| 4267 | 6-N-({2-[(7S*)-5H,6H,7H,8H-imidazo[1,2-a]pyridin-7-ylmethoxy]pyridin-4- |
| | yl}methyl)isoquinoline-1,6-diamine |
| 4268 | 4-chloro-6-N-({2-[(1-methylpiperidin-4-yl)methoxy]pyridin-4-yl}methyl)isoquinoline-1,6- |
| | diamine |
| 4269 | N6-((2-((3-methyl-3-azabicyclo[3.1.0]hexan-6-yl)methoxy)pyridin-4- |
| | yl)methyl)isoquinoline-1,6-diamine |
| 4270 | 6-N-[[2-[(3-methyl-3-azabicyclo[3.2.1]octan-8-yl)methoxy]-4- |
| | pyridyl]methyl]isoquinoline-1,6-diamine |
| 4271 | 6-N-({2-[(1-methylpiperidin-4-yl)methoxy]pyridin-4-yl}methyl)-2,7-naphthyridine-1,6- |
| | diamine |
| 4272 | 4-N-[[2-[(1-methyl-4-piperidyl)methoxy]-4-pyridyl]methyl]-1,7-naphthyridine-4,8- |
| | diamine |
| 4273 | N8-((2-((1-methylpiperidin-4-yl)methoxy)pyridin-4-yl)methyl)-1,7-naphthyridine-4,8- |
| | diamine |
| 4274 | 1-(5-{[(4-{[(1-aminoisoquinolin-5-yl)amino]methyl}pyridin-2-yl)oxy]methyl}-2- |
| | azabicyclo[2.2.1]heptan-2-yl)ethanone |
| 4275 | 1-(5-{[(4-{[(1-aminoisoquinolin-6-yl)amino]methyl}pyridin-2-yl)oxy]methyl}-2- |
| | azabicyclo[2.2.1]heptan-2-yl)ethanone |
| 4276 | 5-N-{[2-({2-methyl-2-azabicyclo[2.2.1]heptan-5-yl}methoxy)pyridin-4- |
| | yl]methyl}isoquinoline-1,5-diamine |
| 4277 | 6-N-{[2-({2-methyl-2-azabicyclo[2.2.1]heptan-5-yl}methoxy)pyridin-4- |
| | yl]methyl}isoquinoline-1,6-diamine |
| 4278 | 5-N-[[2-[(3-methyl-3-azabicyclo[3.2.1]octan-8-yl)methoxy]-4- |
| | pyridyl]methyl]isoquinoline-1,5-diamine |
| 4279 | (5S)-5-{[(4-{[(1-aminoisoquinolin-5-yl)amino]methyl}pyridin-2-yl)oxy]methyl}-3-methyl- |
| | 1,3-oxazolidin-2-one |
| 4280 | (5R)-5-{[(4-{[(1-aminoisoquinolin-5-yl)amino]methyl}pyridin-2-yl)oxy]methyl}-3-methyl- |
| | 1,3-oxazolidin-2-one |
| 4281 | 5-N-[[2-[(1-methyl-4-piperidyl)methoxy]-6-(trifluoromethyl)-4- |
| | pyridyl]methyl]isoquinoline-1,5-diamine |
| 4282 | 5-N-({2-[2-(1-methylimidazol-2-yl)ethoxy]pyridin-4-yl}methyl)isoquinoline-1,5-diamine |
| 4283 | 6-N-({2-[2-(1-methylimidazol-2-yl)ethoxy]pyridin-4-yl}methyl)isoquinoline-1,6-diamine |
| 4284 | 6-N-[(2-{[(1-methylpiperidin-4-yl)methyl]amino}pyridin-4-yl)methyl]isoquinoline-1,6- |
| | diamine |
| 4285 | 4-chloro-6-N-[(2-{[(1-methylpiperidin-4-yl)methyl]amino}pyridin-4- |
| | yl)methyl]isoquinoline-1,6-diamine |
| 4286 | N5-((2-(((1-methylpiperidin-4-yl)methyl)amino)pyridin-4-yl)methyl)isoquinoline-1,5- |
| | diamine |
| 4287 | 6-N-{[3-(pyrrolidin-1-ylmethyl)phenyl]methyl}isoquinoline-1,6-diamine |
| 4288 | 6-N-({3-[2-(4-methylpiperazin-1-yl)ethyl]phenyl}methyl)isoquinoline-1,6-diamine |
| 4289 | 4-(((1-aminoisoquinolin-5-yl)amino)methyl)-N-(1-methylpiperidin-4-yl)picolinamide |
| 4293 | 3-N-({2-[(1-methylpiperidin-4-yl)methoxy]pyridin-4-yl}methyl)-1,7-naphthyridine-3,8- |
| | diamine |
| 4294 | 5-N-[(2-{[(1R,4R)-5-isopropyl-2,5-diazabicyclo[2.2.1]heptan-2-yl]methyl}pyridin-4- |
| | yl)methyl]isoquinoline-1,5-diamine |
| 4295 | 5-N-[(2-{[(1R,4R)-5-isopropyl-2,5-diazabicyclo[2.2.1]heptan-2-yl]methyl}pyridin-4- |
| | yl)methyl]isoquinoline-1,5-diamine |
| 4296 | 4-chloro-6-N-[(2-{[(1-isopropylpiperidin-4-yl)oxy]methyl}pyridin-4- |
| | yl)methyl]isoquinoline-1,6-diamine |
| 4297 | N5-((2-((3-isopropyl-3-azabicyclo[3.2.1]octan-8-yl)methoxy)pyridin-4- |
| | yl)methyl)isoquinoline-1,5-diamine |
| 4298 | N5-[[2-(5,6,7,8-tetrahydroimidazo[1,2-a]pyridin-7-ylmethoxy)-4- |
| | pyridyl]methyl]isoquinoline-1,5-diamine |
| 4299 | 5-N-({2-[(1-isopropylpiperidin-4-yl)methoxy]pyridin-4-yl}methyl)isoquinoline-1,5- |
| | diamine |
| 4300 | 4-chloro-6-N-[(2-{5H,6H,7H,8H-imidazo[1,2-a]pyridin-7-ylmethoxy}pyridin-4- |
| | yl)methyl]isoquinoline-1,6-diamine |
| 4301 | 4-{[(4-{[(1-aminoisoquinolin-5-yl)amino]methyl}pyridin-2-yl)oxy]methyl}-1- |
| | methylpyridin-2-one |
| 4302 | 3-[(4-{[(1-aminoisoquinolin-5-yl)amino]methyl}pyridin-2-yl)amino]-1-(pyrrolidin-1- |
| | yl)propan-1-one |
| 4303 | 4-chloro-6-N-{[2-({5H,6H,7H,8H-imidazo[1,2-a]pyridin-7-ylmethyl}amino)pyridin-4- |
| | yl]methyl}isoquinoline-1,6-diamine |
| 4306 | 5-N-[(2-{5H,6H,7H,8H-imidazo[1,2-a]pyridin-7-yloxy}pyridin-4-yl) methyl]isoquinoline- |
| | 1,5-diamine |
| 4307 | 4-fluoro-6-N-[(2-{5H,6H,7H,8H-imidazo[1,2-a]pyridin-7-ylmethoxy}pyridin-4- |
| | yl)methyl]isoquinoline-1,6-diamine |
| 4308 | 4-fluoro-5-N-[(2-{5H,6H,7H,8H-imidazo[1,2-a]pyridin-7-ylmethoxy}pyridin-4- |
| | yl)methyl]isoquinoline-1,5-diamine |
| 4309 | 4-chloro-6-N-{[2-({2-methyl-5H,6H,7H,8H-imidazo[1,2-a]pyridin-7-yl}methoxy)pyridin-4- |
| | yl]methyl}isoquinoline-1,6-diamine |
| 4319 | 5-N-{[2-({2-methyl-5H,6H,7H,8H-imidazo[1,2-a]pyridin-7-yl}methoxy)pyridin-4- |
| | yl]methyl}isoquinoline-1,5-diamine |
| 4320 | 5-N-{[2-({3-methyl-5H,6H,7H,8H-imidazo[1,2-a]pyridin-7-yl}methoxy)pyridin-4- |
| | yl]methyl}isoquinoline-1,5-diamine |
| 4408 | 6-N-[(2-{5H,6H,7H,8H-imidazo[1,2-a]pyridin-7-ylmethoxy}pyridin-4- |
| | yl)methyl]isoquinoline-1,6-diamine |
| 4409 | 6-N-({2-[2-(1-methylpiperidin-4-yl)ethyl]pyridin-4-yl}methyl)isoquinoline-1,6-diamine |
| 4410 | 6-N-({3-[2-(1-methylpiperidin-4-yl)ethyl]phenyl}methyl)isoquinoline-1,6-diamine |
| 4411 | 6-N-[(2-{5H,6H,7H,8H-[1,2,4]triazolo[4,3-a]pyridin-7-ylmethoxy}pyridin-4- |
| | yl)methyl]isoquinoline-1,6-diamine |
| 4412 | 6-N-({2-[(7R*)-5H,6H,7H,8H-imidazo[1,2-a]pyridin-7-ylmethoxy]pyridin-4- |
| | yl}methyl)isoquinoline-1,6-diamine |
| 4413 | 6-N-{[2-({3-methyl-5H,6H,7H,8H-[1,2,4]triazolo[4,3-a]pyridin-7-yl}methoxy)pyridin-4- |
| | yl]methyl}isoquinoline-1,6-diamine |
| 4414 | 6-N-({2-[1-(1-methylpiperidin-4-yl)ethoxy]pyridin-4-yl}methyl)isoquinoline-1,6-diamine |
| 4415 | 6-N-({2-[(4-fluoro-1-methylpiperidin-4-yl)methoxy]pyridin-4-yl}methyl)isoquinoline-1,6- |
| | diamine |
| 4416 | 4-{[(4-{[(1-aminoisoquinolin-6-yl)amino]methyl}pyridin-2-yl)oxy]methyl}-1- |
| | methylpiperidin-2-one |
| 4417 | 4-{[(4-{[(1-aminoisoquinolin-5-yl)amino]methyl}pyridin-2-yl)oxy]methyl}-1- |
| | methylpiperidin-2-one |
| 4418 | 5-N-[(2-{[(1R,5S)-3-methyl-3-azabicyclo[3.1.0]hexan-6-yl]methoxy}pyridin-4- |
| | yl)methyl]isoquinoline-1,5-diamine |
| 4419 | N5-methyl-N5-((2-((1-methylpiperidin-4-yl)methoxy)pyridin-4-yl)methyl)isoquinoline- |
| | 1,5-diamine |
| 4420 | 6-N-({2-[3-(1-methylimidazol-2-yl)propoxy]pyridin-4-yl}methyl)isoquinoline-1,6-diamine |
| 4421 | 5-N-({2-[3-(1-methylimidazol-2-yl)propoxy]pyridin-4-yl}methyl)isoquinoline-1,5-diamine |
| 4422 | 6-N-({2-[(1-methylimidazol-2-yl)methoxy]pyridin-4-yl}methyl)isoquinoline-1,6-diamine |
| 4423 | N5-((2-((1-methyl-1H-imidazol-2-yl)methoxy)pyridin-4-yl)methyl)isoquinoline-1,5- |
| | diamine |
| 4424 | N5-((2-((1,4-dimethylpiperidin-4-yl)methoxy)pyridin-4-yl)methyl)isoquinoline-1,5- |
| | diamine |
| 4425 | 6-N-({2-[(1,4-dimethylpiperidin-4-yl)methoxy]pyridin-4-yl}methyl)isoquinoline-1,6- |
| | diamine |
| 4426 | 5-N-{[2-(4-methylpiperazin-1-yl)pyridin-4-yl]methyl}isoquinoline-1,5-diamine |
| 4427 | 4-chloro-6-N-{[2-(4-methylpiperazin-1-yl)pyridin-4-yl]methyl}isoquinoline-1,6-diamine |
| 4428 | 3-[(4-{[(1-amino-4-chloroisoquinolin-6-yl)amino]methyl}pyridin-2-yl)amino]-1- |
| | (pyrrolidin-1-yl)propan-1-one |
| 4429 | 6-N-[(2-{5H,6H,7H,8H-imidazo[1,2-a]pyridin-7-ylmethoxy}pyridin-4-yl)methyl]-2,7- |
| | naphthyridine-1,6-diamine |
| 4430 | (S*)-N5-((2-((5,6,7,8-tetrahydroimidazo[1,2-a]pyridin-7-yl)methoxy)pyridin-4- |
| | yl)methyl)isoquinoline-1,5-diamine |
| 4431 | (R*)-N5-((2-((5,6,7,8-tetrahydroimidazo[1,2-a]pyridin-7-yl)methoxy)pyridin-4- |
| | yl)methyl)isoquinoline-1,5-diamine |
| 4432 | 4-chloro-6-N-[(2-{5H,6H,7H,8H-imidazo[1,2-a]pyridin-7-yloxy}pyridin-4- |
| | yl)methyl]isoquinoline-1,6-diamine |
| 4433 | 4-chloro-6-N-[(2-{imidazo[1,2-a]pyridin-7-ylmethoxy}pyridin-4-yl)methyl]isoquinoline- |
| | 1,6-diamine |
| 4434 | 6-N-({2-[3-(pyrrolidin-1-ylmethyl)phenyl]pyridin-4-yl}methyl)isoquinoline-1,6-diamine |
| 4435 | 6-N-({2-[3-(1-methylazetidin-3-yl)azetidin-1-yl]pyridin-4-yl}methyl)isoquinoline-1,6- |
| | diamine |
| 4436 | 5-N-[(2-{5H,6H,8H-imidazo[1,2-a]pyrazin-7-yl}pyridin-4-yl)methyl]isoquinoline-1,5- |
| | diamine |
| 4437 | 4-chloro-6-N-[(2-{5H,6H,8H-imidazo[1,2-a]pyrazin-7-yl}pyridin-4-yl)methyl]isoquinoline- |
| | 1,6-diamine |
| 4438 | (S*)-4-chloro-N6-((2-((5,6,7,8-tetrahydroimidazo[1,2-a]pyridin-7-yl)methoxy)pyridin-4- |
| | yl)methyl)isoquinoline-1,6-diamine |
| 4439 | (R*)-4-chloro-N6-((2-((5,6,7,8-tetrahydroimidazo[1,2-a]pyridin-7-yl)methoxy)pyridin-4- |
| | yl)methyl)isoquinoline-1,6-diamine |
| 4440 | 4-chloro-N6-((2-((3-methyl-1,4,5,6-tetrahydrocyclopenta[c]pyrazol-6-yl)amino)pyridin- |
| | 4-yl)methyl)isoquinoline-1,6-diamine |
| 4441 | 3-{[(1-aminoisoquinolin-5-yl)amino]methyl}-N-(3-hydroxypropyl)-N-[(1- |
| | isopropylpiperidin-4-yl)methyl]benzamide |
| 4442 | (R*)-4-chloro-N6-((2-((2-methyl-5,6,7,8-tetrahydroimidazo[1,2-a]pyridin-7- |
| | yl)methoxy)pyridin-4-yl)methyl)isoquinoline-1,6-diamine |
| 4443 | 4-chloro-N6-((2-((5,6,7,8-tetrahydroimidazo[1,2-a]pyridin-8-yl)methoxy)pyridin-4- |
| | yl)methyl)isoquinoline-1,6-diamine |
| 4444 | (S*)-N5-((2-((3-methyl-5,6,7,8-tetrahydroimidazo[1,2-a]pyridin-7-yl)methoxy)pyridin-4- |
| | yl)methyl)isoquinoline-1,5-diamine |
| 4445 | (R*)-N5-((2-((3-methyl-5,6,7,8-tetrahydroimidazo[1,2-a]pyridin-7-yl)methoxy)pyridin-4- |
| | yl)methyl)isoquinoline-1,5-diamine |
| 4446 | (S*)-N5-((2-((2-methyl-5,6,7,8-tetrahydroimidazo[1,2-a]pyridin-7-yl)methoxy)pyridin-4- |
| | yl)methyl)isoquinoline-1,5-diamine |
| 4447 | (R*)-N5-((2-((2-methyl-5,6,7,8-tetrahydroimidazo[1,2-a]pyridin-7-yl)methoxy)pyridin-4- |
| | yl)methyl)isoquinoline-1,5-diamine |
| 4448 | (7-(((4-(((1-amino-4-chloroisoquinolin-6-yl)amino)methyl)pyridin-2-yl)oxy)methyl)- |
| | 5,6,7,8-tetrahydroimidazo[1,2-a]pyridin-3-yl)methanol |
| 4449 | (7-(((4-(((1-amino-4-chloroisoquinolin-6-yl)amino)methyl)pyridin-2-yl)oxy)methyl)- |
| | 5,6,7,8-tetrahydroimidazo[1,2-a]pyridin-2-yl)methanol |
| 4450 | (S*)-4-chloro-N6-((2-((5,6,7,8-tetrahydroimidazo[1,2-a]pyridin-8-yl)methoxy)pyridin-4- |
| | yl)methyl)isoquinoline-1,6-diamine |
| 4451 | (R*)-4-chloro-N6-((2-((5,6,7,8-tetrahydroimidazo[1,2-a]pyridin-8-yl)methoxy)pyridin-4- |
| | yl)methyl)isoquinoline-1,6-diamine |
| 4452 | 5-(2-(2-((5,6,7,8-tetrahydroimidazo[1,2-a]pyridin-7-yl)methoxy)pyridin-4- |
| | yl)ethyl)isoquinolin-1-amine |
| 4453 | 7-(((4-(((1-amino-4-chloroisoquinolin-6-yl)amino)methyl)pyridin-2-yl)oxy)methyl)- |
| | 5,6,7,8-tetrahydroimidazo[1,2-a]pyridine-3-carboxylic acid |
| 4454 | 3-chloro-N-((2-((1-methylpiperidin-4-yl)methoxy)pyridin-4-yl)methyl)-1H-indazol-4- |
| | amine |
| 4455 | 1-(5-(methyl(2-((1-methylpiperidin-4-yl)methoxy)pyridin-4-yl)amino)pyridin-3-yl)ethan- |
| | 1-one |
| 4456 | 7-(methyl(2-((1-methylpiperidin-4-yl)methoxy)pyridin-4-yl)amino)quinoxalin-2(1H)-one |
| 4457 | 4-(5-methyl-1,3,4-oxadiazol-2-yl)-N-((2-((1-methylpiperidin-4-yl)methoxy)pyridin-4- |
| | yl)methyl)aniline |
| 4458 | N3,N3-dimethyl-N6-((2-((1-methylpiperidin-4-yl)methoxy)pyridin-4- |
| | yl)methyl)pyridazine-3,6-diamine |
| 4459 | N-((2-((1-methylpiperidin-4-yl)methoxy)pyridin-4-yl)methyl)-1H-pyrrolo[2,3-b]pyridin-4- |
| | amine |
| 4460 | 5-(methyl(2-((1-methylpiperidin-4-yl)methoxy)pyridin-4-yl)amino)-1H-pyrrolo[2,3- |
| | b]pyridine-3-carbonitrile |
| 4461 | 4-fluoro-N5-((2-((1-methylpiperidin-4-yl)methoxy)pyridin-4-yl)methyl)isoquinoline-1,5- |
| | diamine |
| 4462 | 8-methoxy-N-((2-((1-methylpiperidin-4-yl)methoxy)pyridin-4-yl)methyl)isoquinolin-6- |
| | amine |
| 4463 | N-methyl-6-(methyl(2-((1-methylpiperidin-4-yl)methoxy)pyridin-4- |
| | yl)amino)isoquinoline-8-carboxamide |
| 4464 | 8-methyl-N-((2-((1-methylpiperidin-4-yl)methoxy)pyridin-4-yl)methyl)isoquinolin-6- |
| | amine |
| 4465 | 2-methyl-N-((2-((1-methylpiperidin-4-yl)methoxy)pyridin-4-yl)methyl)-1H-pyrrolo[2,3- |
| | b]pyridin-4-amine |
| 4466 | 5-fluoro-N-((2-((1-methylpiperidin-4-yl)methoxy)pyridin-4-yl)methyl)isoquinolin-6- |
| | amine |
| 4467 | N5-((2-((3-(difluoromethyl)-5,6,7,8-tetrahydro-[1,2,4]triazolo[4,3-a]pyridin-7- |
| | yl)methoxy)pyridin-4-yl)methyl)isoquinoline-1,5-diamine |
| 4468 | N5-((2-((5-cyclopropyl-4-methyl-4H-1,2,4-triazol-3-yl)methoxy)pyridin-4- |
| | yl)methyl)isoquinoline-1,5-diamine |
| 4469 | 8-fluoro-N5-((2-((1-methylpiperidin-4-yl)methoxy)pyridin-4-yl)methyl)isoquinoline-1,5- |
| | diamine |
| 4470 | 8-methoxy-N5-((2-((1-methylpiperidin-4-yl)methoxy)pyridin-4-yl)methyl)isoquinoline- |
| | 1,5-diamine |
| 4471 | N6-((2-(3-(trifluoromethyl)-5,6-dihydro-[1,2,4]triazolo[4,3-a]pyrazin-7(8H)-yl)pyridin-4- |
| | yl)methyl)isoquinoline-1,6-diamine |
| 4472 | N5-((2-(3-(trifluoromethyl)-5,6-dihydro-[1,2,4]triazolo[4,3-a]pyrazin-7(8H)-yl)pyridin-4- |
| | yl)methyl)isoquinoline-1,5-diamine |
| 5434 | N6-((2-(3-(pyrrolidin-1-ylmethyl)phenyl)pyridin-4-yl)methyl)isoquinoline-1,6-diamine |
| 5435 | N6-((2-(1′-methyl-[3,3′-biazetidin]-1-yl)pyridin-4-yl)methyl)isoquinoline-1,6-diamine |
| 5436 | N5-((2-(5,6-dihydroimidazo[1,2-a]pyrazin-7(8H)-yl)pyridin-4-yl)methyl)isoquinoline-1,5- |
| | diamine |
| 5437 | 4-chloro-N6-((2-(5,6-dihydroimidazo[1,2-a]pyrazin-7(8H)-yl)pyridin-4- |
| | yl)methyl)isoquinoline-1,6-diamine |
| 5441 | 3-(((1-aminoisoquinolin-5-yl)amino)methyl)-N-(3-hydroxypropyl)-N-((1- |
| | isopropylpiperidin-4-yl)methyl)benzamide |
| 5442 | 2-(3-(((1-amino-4-chloroisoquinolin-6-yl)amino)methyl)phenyl)-1-(5,6- |
| | dihydroimidazo[1,2-a]pyrazin-7(8H)-yl)ethan-1-one |
| 5443 | N-((2-(3-(pyrrolidin-1-ylmethyl)phenyl)pyridin-4-yl)methyl)isoquinolin-6-amine |
| 8458 | N5-((2-(3-(trifluoromethyl)-5,6,7,8-tetrahydro-[1,2,4]triazolo[4,3-a]pyridin-7-yl)oxazol- |
| | 5-yl)methyl)isoquinoline-1,5-diamine |
| 8459 | N5-((4-(3-(trifluoromethyl)-5,6-dihydro-[1,2,4]triazolo[4,3-a]pyrazin-7(8H)-yl)thiophen- |
| | 2-yl)methyl)isoquinoline-1,5-diamine |
| 9001 | N5-((5-(3-(trifluoromethyl)-5,6-dihydro-[1,2,4]triazolo[4,3-a]pyrazin-7(8H)-yl)pyridin-2- |
| | yl)methyl)isoquinoline-1,5-diamine |
| 9002 | N5-((5-(3-(trifluoromethyl)-5,6-dihydro-[1,2,4]triazolo[4,3-a]pyrazin-7(8H)-yl)pyrazin-2- |
| | yl)methyl)isoquinoline-1,5-diamine |
| 9003 | N5-((2-(3-(trifluoromethyl)-5,6-dihydro-[1,2,4]triazolo[4,3-a]pyrazin-7(8H)-yl)pyrimidin- |
| | 5-yl)methyl)isoquinoline-1,5-diamine |
| 9004 | N5-((6-(3-(trifluoromethyl)-5,6-dihydro-[1,2,4]triazolo[4,3-a]pyrazin-7(8H)-yl)pyridazin- |
| | 3-yl)methyl)isoquinoline-1,5-diamine |
| 9005 | N5-((3-methyl-5-(3-(trifluoromethyl)-5,6-dihydro-[1,2,4]triazolo[4,3-a]pyrazin-7(8H)- |
| | yl)pyridin-2-yl)methyl)isoquinoline-1,5-diamine |
| 9006 | N5-((2,4-dimethyl-6-(3-(trifluoromethyl)-5,6-dihydro-[1,2,4]triazolo[4,3-a]pyrazin- |
| | 7(8H)-yl)pyridin-3-yl)methyl)isoquinoline-1,5-diamine |
| 9007 | N5-((3-methyl-5-(8-methyl-3-(trifluoromethyl)-5,6-dihydro-[1,2,4]triazolo[4,3-a]pyrazin- |
| | 7(8H)-yl)pyridin-2-yl)methyl)isoquinoline-1,5-diamine |
| 9008 | N5-((3-fluoro-5-(8-methyl-3-(trifluoromethyl)-5,6-dihydro-[1,2,4]triazolo[4,3-a]pyrazin- |
| | 7(8H)-yl)pyridin-2-yl)methyl)isoquinoline-1,5-diamine |
| 10001 | 5-(2-(6-(3-(difluoromethyl)-5,6-dihydro-[1,2,4]triazolo[4,3-a]pyrazin-7(8H)-yl)pyridin-3- |
| | yl)pyrrolidin-1-yl)isoquinolin-1-amine |
| 10002 | 5-(2-(6-(3-(trifluoromethyl)-5,6-dihydro-[1,2,4]triazolo[4,3-a]pyrazin-7(8H)-yl)pyridin-3- |
| | yl)pyrrolidin-1-yl)isoquinolin-1-amine |
| 10003 | (S*)-5-(2-(6-(3-(trifluoromethyl)-5,6-dihydro-[1,2,4]triazolo[4,3-a]pyrazin-7(8H)- |
| | yl)pyridin-3-yl)pyrrolidin-1-yl)isoquinolin-1-amine |
| 10004 | (R*)-5-(2-(6-(3-(trifluoromethyl)-5,6-dihydro-[1,2,4]triazolo[4,3-a]pyrazin-7(8H)- |
| | yl)pyridin-3-yl)pyrrolidin-1-yl)isoquinolin-1-amine |

TABLE 12
¹H NMR data of examples (solvent d6 DMSO unless otherwise indicated)

| Example | |
|---|---|
| Number | NMR write-up |
| 1001 | 1.58-1.67 (2H, m), 1.90-1.97 (2H, m), 2.09-2.16 (2H, m), 2.17 (3H, s), 2.59-2.65 |
| | (2H, m), 4.27 (2H, d, J = 5.7 Hz), 4.89-4.99 (1H, m), 6.27-6.31 (2H, m), 6.53 (1H, d, |
| | J = 2.3 Hz), 6.57 (1H, d, J = 5.9 Hz), 6.66 (1H, t, J = 5.9 Hz), 6.75 (1H, d, J = 8.5 Hz), |
| | 6.86 (1H, dd, J = 9.1, 2.4 Hz), 7.55 (1H, d, J = 5.8 Hz), 7.69 (1H, dd, J = 8.5, 2.5 Hz), |
| | 7.85 (1H, d, J = 9.0 Hz), 8.17 (1H, d, J = 2.5 Hz) |
| 1002 | 1.53-1.66 (2H, m), 1.85-1.94 (2H, m), 2.13-2.23 (5H, m), 2.58-2.65 (2H, m), |
| | 4.30 (1H, tt, J = 8.2, 3.9 Hz), 4.37 (2H, d, J = 6.0 Hz), 6.46-6.51 (3H, m), 6.68 (1H, t, |
| | J = 6.0 Hz), 6.85-6.90 (2H, m), 7.09-7.14 (1H, m), 7.20 (1H, d, J = 6.1 Hz), 7.25- |
| | 7.31 (3H, m), 7.74 (1H, d, J = 6.1 Hz) |
| 1003 | 1.53-1.64 (2H, m), 1.86-1.94 (2H, m), 2.10-2.18 (5H, m), 2.56-2.62 (2H, m), |
| | 4.26-4.35 (3H, m), 6.26 (2H, s), 6.31 (1H, t, J = 5.9 Hz), 6.71 (1H, d, J = 5.7 Hz), 6.89- |
| | 6.92 (2H, m), 7.01 (1H, d, J = 2.2 Hz), 7.11 (1H, dd, J = 8.8, 2.2 Hz), 7.32-7.35 (2H, |
| | m), 7.40 (1H, d, J = 8.8 Hz), 7.49 (1H, d, J = 5.7 Hz) |
| 1004 | 1.57-1.67 (2H, m), 1.88-1.94 (2H, m), 2.15-2.24 (5H, m), 2.59-2.67 (2H, m), |
| | 4.23 (2H, d, J = 6.0 Hz), 4.28-4.36 (1H, m), 6.06 (1H, t, J = 6.0 Hz), 6.87 (1H, d, J = |
| | 2.6 Hz), 6.89-6.93 (2H, m), 7.28-7.32 (2H, m), 7.41 (1H, d, J = 2.8 Hz), 7.88 (1H, d, |
| | J = 2.6 Hz), 11.44 (1H, d, J = 1.8 Hz) |
| 1005 | 1.19-1.34 (2H, m), 1.63-1.74 (3H, m), 1.83-1.92 (2H, m), 2.17 (3H, s), 2.74-2.82 |
| | (2H, m), 4.07 (2H, d, J = 6.1 Hz), 4.29 (2H, d, J = 5.8 Hz), 6.40 (2H, s), 6.54 (1H, d, J = |
| | 2.4 Hz), 6.58 (1H, d, J = 5.9 Hz), 6.68-6.74 (1H, m), 6.78 (1H, d, J = 8.5 Hz), 6.87 |
| | (1H, dd, J = 9.1, 2.4 Hz), 7.54 (1H, d, J = 5.9 Hz), 7.70 (1H, dd, J = 8.5, 2.5 Hz), 7.86 |
| | (1H, d, J = 9.0 Hz), 8.17 (1H, d, J = 2.4 Hz) |
| 1006 | 1.67-1.78 (1H, m), 2.10-2.16 (1H, m), 2.31-2.41 (1H, m), 2.45-2.51 (1H, m, |
| | partially obscured by DMSO), 2.92 (1H, ddd, J = 16.3, 5.0, 1.5 Hz), 3.84-3.92 (1H, |
| | m), 4.05-4.11 (1H, m), 4.25 (2H, d, J = 6.6 Hz), 4.31 (2H, d, J = 5.8 Hz), 6.52-6.58 |
| | (3H, m), 6.61 (1H, d, J = 6.0 Hz), 6.77-6.82 (2H, m), 6.84 (1H, d, J = 8.5 Hz), 6.89 |
| | (1H, dd, J = 9.1, 2.4 Hz), 6.99 (1H, d, J = 1.3 Hz), 7.53 (1H, d, J = 6.0 Hz), 7.73 (1H, |
| | dd, J = 8.5, 2.5 Hz), 7.89 (1H, d, J = 9.0 Hz), 8.20 (1H, d, J = 2.4 Hz) |
| 1007 | 2.13 (3H, s), 2.22-2.40 (8H, m), 3.41 (2H, s), 4.33 (2H, d, J = 5.8 Hz), 6.28 (2H, s), |
| | 6.48 (1H, d, J = 2.3 Hz), 6.53 (1H, d, J = 5.9 Hz), 6.72 (1H, t, J = 5.9 Hz), 6.88 (1H, dd, |
| | J = 9.0, 2.3 Hz), 7.22-7.26 (2H, m), 7.31-7.35 (2H, m), 7.53 (1H, d, J = 5.9 Hz), 7.84 |
| | (1H, d, J = 9.0 Hz) |
| 1008 | 1.22-1.31 (2H, m), 1.64-1.72 (3H, m), 1.81-1.90 (2H, m), 2.16 (3H, s), 2.74-2.81 |
| | (2H, m), 4.08 (2H, d, J = 6.1 Hz), 4.33 (2H, d, J = 5.6 Hz), 6.68 (1H, d, J = 2.3 Hz), 6.79 |
| | (1H, d, J = 8.5 Hz), 7.02 (1H, t, J = 5.7 Hz), 7.11 (1H, dd, J = 8.9, 2.3 Hz), 7.38 (1H, d, |
| | J = 5.8 Hz), 7.72 (1H, dd, J = 8.5, 2.5 Hz), 7.75 (1H, d, J = 8.9 Hz), 8.18 (1H, d, J = 5.8 |
| | Hz), 8.20 (1H, d, J = 2.5 Hz), 8.86 (1H, s) |
| 1009 | 1.21-1.32 (2H, m), 1.64-1.73 (3H, m), 1.81-1.94 (2H, m), 2.17 (3H, s), 2.74-2.82 |
| | (2H, m), 4.06 (2H, d, J = 6.1 Hz), 4.38 (2H, d, J = 5.7 Hz), 6.50 (2H, s), 6.54 (1H, d, J = |
| | 7.7 Hz), 6.65 (1H, t, J = 5.9 Hz), 6.74 (1H, d, J = 8.5 Hz), 7.12-7.18 (2H, m), 7.32 (1H, |
| | d, J = 8.3 Hz), 7.69 (1H, dd, J = 8.5, 2.5 Hz), 7.74 (1H, d, J = 6.1 Hz), 8.16 (1H, d, J = 2.4 Hz) |
| 1010 | 1.02 (3H, t, J = 7.2 Hz), 1.23-1.35 (2H, m), 1.69-1.80 (3H, m), 1.95-2.09 (2H, m), |
| | 2.39-2.46 (2H, m), 2.91-3.00 (2H, m), 4.08 (2H, d, J = 6.2 Hz), 4.30 (2H, d, J = 5.8 |
| | Hz), 6.55-6.63 (4H, m), 6.76-6.82 (2H, m), 6.89 (1H, dd, J = 9.1, 2.4 Hz), 7.53 (1H, |
| | d, J = 6.0 Hz), 7.71 (1H, dd, J = 8.5, 2.5 Hz), 7.89 (1H, d, J = 9.0 Hz), 8.18 (1H, d, J = 2.4 Hz) |
| 1011 | 1.44-1.55 (2H, m), 1.81-1.89 (2H, m), 1.96-2.04 (2H, m), 2.13 (3H, s), 2.57-2.62 |
| | (2H, m), 3.33-3.38 (1H, m), 4.50 (2H, s), 5.21 (2H, s), 6.61 (2H, s), 6.80 (1H, d, J = |
| | 5.8 Hz), 7.12 (1H, dd, J = 9.1, 2.6 Hz), 7.19 (1H, d, J = 2.6 Hz), 7.34-7.38 (2H, m), |
| | 7.44-7.49 (2H, m), 7.72 (1H, d, J = 5.8 Hz), 8.10 (1H, d, J = 9.1 Hz) |
| 1012 | 0.95 (6H, d, J = 6.5 Hz), 1.15-1.28 (2H, m), 1.59-1.75 (3H, m), 2.02-2.13 (2H, m), |
| | 2.61-2.70 (1H, m), 2.72-2.81 (2H, m), 4.06 (2H, d, J = 6.1 Hz), 4.28 (2H, d, J = 5.4 |
| | Hz), 6.26-6.32 (2H, m), 6.53 (1H, d, J = 2.3 Hz), 6.57 (1H, d, J = 5.9 Hz), 6.67 (1H, t, |
| | J = 5.9 Hz), 6.78 (1H, d, J = 8.5 Hz), 6.84-6.88 (1H, m), 7.55 (1H, d, J = 5.8 Hz), 7.66- |
| | 7.73 (1H, m), 7.85 (1H, d, J = 9.1 Hz), 8.17 (1H, d, J = 2.4 Hz) |
| 1013 | Methanol-d4 1.36-1.49 (2H, m), 1.81-1.90 (3H, m), 2.03-2.12 (2H, m), 2.30 (3H, |
| | s), 2.90-2.97 (2H, m), 4.25 (2H, d, J = 6.2 Hz), 4.44 (2H, s), 6.66 (1H, d, J = 2.4 Hz), |
| | 6.77 (1H, d, J = 6.2 Hz), 6.98 (1H, dd, J = 9.1, 2.4 Hz), 7.51 (1H, d, J = 6.2 Hz), 7.89 |
| | (1H, d, J = 9.0 Hz), 8.60 (2H, s), 3 × NH not observed |
| 1014 | 4.30 (2H, d, J = 5.7 Hz), 5.40 (2H, s), 6.25-6.33 (2H, m), 6.53 (1H, d, J = 2.3 Hz), |
| | 6.57 (1H, d, J = 5.8 Hz), 6.68 (1H, t, J = 5.9 Hz), 6.86 (1H, dd, J = 9.0, 2.3 Hz), 6.94 |
| | (1H, d, J = 8.5 Hz), 7.37-7.42 (2H, m), 7.55 (1H, d, J = 5.8 Hz), 7.78 (1H, dd, J = 8.5, |
| | 2.5 Hz), 7.85 (1H, d, J = 9.1 Hz), 8.18 (1H, d, J = 2.4 Hz), 8.52-8.57 (2H, m) |
| 1015 | 1.51-1.62 (1H, m), 1.90-1.97 (1H, m), 2.01-2.08 (1H, m), 2.20-2.31 (1H, m), |
| | 2.31-2.37 (1H, m), 2.80 (3H, s), 3.24-3.30 (2H, m), 4.12 (2H, d, J = 6.4 Hz), 4.29 |
| | (2H, d, J = 5.7 Hz), 6.25-6.34 (2H, m), 6.53 (1H, d, J = 2.4 Hz), 6.57 (1H, d, J = 5.8 |
| | Hz), 6.67 (1H, t, J = 5.9 Hz), 6.80 (1H, d, J = 8.5 Hz), 6.86 (1H, dd, J = 9.0, 2.4 Hz), |
| | 7.55 (1H, d, J = 5.8 Hz), 7.72 (1H, dd, J = 8.5, 2.5 Hz), 7.85 (1H, d, J = 9.1 Hz), 8.18 |
| | (1H, d, J = 2.4 Hz) |
| 1016 | 1.08-1.19 (2H, m), 1.61-1.69 (2H, m), 1.76-1.86 (1H, m), 2.44-2.51 (2H, m), |
| | 2.94-2.99 (2H, m), 4.05 (2H, d, J = 6.5 Hz), 4.28 (2H, d, J = 5.8 Hz), 6.31 (2H, s), |
| | 6.53 (1H, d, J = 2.3 Hz), 6.57 (1H, d, J = 5.8 Hz), 6.67 (1H, t, J = 5.9 Hz), 6.77 (1H, d, |
| | J = 8.5 Hz), 6.86 (1H, dd, J = 9.0, 2.4 Hz), 7.55 (1H, d, J = 5.8 Hz), 7.70 (1H, dd, J = 8.5, |
| | 2.5 Hz), 7.85 (1H, d, J = 9.0 Hz), 8.17 (1H, d, J = 2.4 Hz), 1 × NH not observed. |
| 1017 | 1.07 (6H, s), 1.22-1.34 (2H, m), 1.60-1.70 (3H, m), 2.05-2.13 (2H, m), 2.17 (2H, |
| | s), 2.89-2.96 (2H, m), 4.01 (1H, s), 4.07 (2H, d, J = 6.1 Hz), 4.29 (2H, d, J = 5.8 Hz), |
| | 6.36 (2H, s), 6.54 (1H, d, J = 2.3 Hz), 6.58 (1H, d, J = 6.1 Hz), 6.70 (1H, t, J = 5.9 Hz), |
| | 6.75-6.80 (1H, m), 6.87 (1H, dd, J = 9.0, 2.3 Hz), 7.55 (1H, d, J = 5.9 Hz), 7.70 (1H, |
| | dd, J = 8.5, 2.5 Hz), 7.86 (1H, d, J = 9.0 Hz), 8.17 (1H, d, J = 2.5 Hz) |
| 1018 | 1.20-1.33 (2H, m), 1.62-1.74 (3H, m), 1.86-1.95 (2H, m), 2.18 (3H, s), 2.76-2.82 |
| | (2H, m), 2.88-2.94 (2H, m), 2.95-3.02 (2H, m), 4.04 (2H, d, J = 6.1 Hz), 6.65-6.72 |
| | (3H, m), 6.80 (1H, d, J = 5.8 Hz), 7.34 (1H, dd, J = 8.5, 1.8 Hz), 7.46 (1H, d, J = 1.8 |
| | Hz), 7.57 (1H, dd, J = 8.5, 2.5 Hz), 7.74 (1H, d, J = 5.8 Hz), 7.94 (1H, d, J = 2.5 Hz), |
| | 8.09 (1H, d, J = 8.5 Hz) |
| 1019 | 1.26-1.38 (2H, m), 1.66-1.78 (3H, m), 2.02-2.14 (2H, m), 2.27 (3H, s), 2.83-2.92 |
| | (4H, m), 3.13-3.20 (2H, m), 4.07 (2H, d, J = 6.1 Hz), 6.69-6.77 (3H, m), 7.05-7.09 |
| | (1H, m), 7.32-7.38 (1H, m), 7.39-7.43 (1H, m), 7.60 (1H, dd, J = 8.5, 2.5 Hz), 7.83 |
| | (1H, d, J = 6.1 Hz), 7.95 (1H, d, J = 2.5 Hz), 8.05 (1H, d, J = 8.3 Hz) |
| 1020 | 1.20-1.31 (2H, m), 1.64-1.71 (3H, m), 1.75-1.87 (2H, m), 2.14 (3H, s), 2.71-2.78 |
| | (2H, m), 4.07 (2H, d, J = 6.1 Hz), 4.25 (2H, d, J = 6.0 Hz), 6.06 (1H, t, J = 6.1 Hz), 6.77 |
| | (1H, d, J = 8.5 Hz), 6.93 (1H, d, J = 2.6 Hz), 7.42 (1H, d, J = 2.8 Hz), 7.72 (1H, dd, J = |
| | 8.6, 2.5 Hz), 7.88 (1H, d, J = 2.6 Hz), 8.17 (1H, d, J = 2.4 Hz), 11.47 (1H, s) |
| 1021 | 1.21-1.31 (2H, m), 1.63-1.71 (3H, m), 1.79-1.86 (2H, m), 2.13 (3H, s), 2.72-2.77 |
| | (2H, m), 4.07 (2H, d, J = 6.1 Hz), 4.30 (2H, d, J = 5.8 Hz), 6.49 (1H, d, J = 2.3 Hz), 6.78 |
| | (1H, d, J = 8.5 Hz), 6.86 (1H, dd, J = 8.9, 2.4 Hz), 6.92 (1H, t, J = 5.8 Hz), 7.20 (2H, s), |
| | 7.69 (1H, dd, J = 8.5, 2.4 Hz), 7.85 (1H, d, J = 9.0 Hz), 8.13 (1H, s), 8.16 (1H, d, J = 2.4 Hz) |
| 1022 | 1.19-1.31 (2H, m), 1.61-1.70 (3H, m), 1.78-1.86 (2H, m), 2.13 (3H, s), 2.72-2.78 |
| | (2H, m), 4.06 (2H, d, J = 6.1 Hz), 4.40 (2H, d, J = 6.3 Hz), 6.68 (1H, dd, J = 7.9, 1.1 |
| | Hz), 6.74 (1H, d, J = 8.5 Hz), 6.79 (1H, t, J = 6.4 Hz), 7.13-7.20 (1H, m), 7.26 (1H, dd, |
| | J = 8.3, 1.1 Hz), 7.55 (2H, s), 7.71 (1H, dd, J = 8.5, 2.4 Hz), 8.16 (1H, d, J = 2.4 Hz), |
| | 8.33 (1H, s) |
| 1023 | 1.66-1.77 (1H, m), 2.09-2.16 (1H, m), 2.33-2.41 (1H, m), 2.45-2.50 (1H, m), |
| | 2.92 (1H, dd, J = 16.4, 5.0 Hz), 3.88 (1H, app td, J = 12.0, 4.7 Hz), 4.04-4.11 (1H, |
| | m), 4.25 (2H, d, J = 6.6 Hz), 4.30 (2H, d, J = 5.7 Hz), 6.35 (2H, s), 6.54 (1H, d, J = 2.3 |
| | Hz), 6.58 (1H, d, J = 5.9 Hz), 6.70 (1H, t, J = 5.8 Hz), 6.80 (1H, s), 6.84 (1H, d, J = 8.5 |
| | Hz), 6.87 (1H, dd, J = 9.0, 2.3 Hz), 6.99 (1H, s), 7.55 (1H, d, J = 5.8 Hz), 7.73 (1H, dd, |
| | J = 8.5, 2.5 Hz), 7.86 (1H, d, J = 9.0 Hz), 8.20 (1H, d, J = 2.4 Hz) |
| 1024 | 1.66-1.77 (1H, m), 2.09-2.16 (1H, m), 2.32-2.42 (1H, m), 2.45-2.49 (1H, m), |
| | 2.92 (1H, dd, J = 16.2, 5.0 Hz), 3.88 (1H, app td, J = 11.9, 4.7 Hz), 4.03-4.11 (1H, |
| | m), 4.25 (2H, d, J = 6.6 Hz), 4.30 (2H, d, J = 5.7 Hz), 6.33 (2H, s), 6.54 (1H, d, J = 2.3 |
| | Hz), 6.57 (1H, d, J = 5.9 Hz), 6.69 (1H, t, J = 5.8 Hz), 6.80 (1H, d, J = 1.2 Hz), 6.84 (1H, |
| | d, J = 8.5 Hz), 6.87 (1H, dd, J = 9.0, 2.3 Hz), 6.99 (1H, s), 7.55 (1H, d, J = 5.8 Hz), 7.73 |
| | (1H, dd, J = 8.5, 2.5 Hz), 7.85 (1H, d, J = 9.0 Hz), 8.19 (1H, d, J = 2.4 Hz) |
| 1025 | Methanol-d4 2.13-2.22 (2H, m), 2.89 (2H, t, J = 7.6 Hz), 3.62 (3H, s), 4.30 (2H, t, J = |
| | 6.1 Hz), 4.43 (2H, s), 6.72 (1H, d, J = 2.4 Hz), 6.80 (1H, d, J = 8.5 Hz), 6.82-6.85 (2H, |
| | m), 6.95 (1H, d, J = 1.4 Hz), 7.06 (1H, dd, J = 9.1, 2.4 Hz), 7.39 (1H, d, J = 6.7 Hz), |
| | 7.73 (1H, dd, J = 8.5, 2.5 Hz), 8.00 (1H, d, J = 9.1 Hz), 8.15 (1H, d, J = 2.5 Hz), 3 × NH |
| | not observed |
| 1026 | 1.20-1.32 (2H, m), 1.64-1.72 (3H, m), 1.79-1.87 (2H, m), 2.14 (3H, s), 2.72-2.79 |
| | (2H, m), 4.08 (2H, d, J = 6.0 Hz), 4.34 (2H, d, J = 5.6 Hz), 4.74 (2H, d, J = 5.2 Hz), 5.18 |
| | (1H, t, J = 5.4 Hz), 6.77-6.82 (2H, m), 7.05 (1H, t, J = 5.7 Hz), 7.10 (1H, dd, J = 8.9, |
| | 2.1 Hz), 7.73 (1H, dd, J = 8.5, 2.5 Hz), 7.77 (1H, d, J = 8.9 Hz), 8.17-8.23 (2H, m), |
| | 8.79 (1H, s) |
| 1027 | 1.20-1.32 (2H, m), 1.63-1.74 (3H, m), 1.78-1.87 (2H, m), 2.14 (3H, s), 2.72-2.79 |
| | (2H, m), 3.93 (3H, s), 4.09 (2H, d, J = 6.0 Hz), 4.19 (2H, d, J = 5.6 Hz), 6.25-6.29 (2H, |
| | m), 6.32 (1H, d, J = 8.0 Hz), 6.44 (1H, d, J = 2.3 Hz), 6.53 (1H, t, J = 5.9 Hz), 6.56 (1H, |
| | d, J = 5.9 Hz), 6.85 (1H, dd, J = 9.0, 2.4 Hz), 7.53-7.56 (2H, m), 7.84 (1H, d, J = 9.0 Hz) |
| 1028 | 1.69-1.81 (1H, m), 2.11-2.20 (1H, m), 2.39-2.47 (2H, m), 2.96 (1H, dd, J = 16.4, |
| | 5.0 Hz), 3.91-4.00 (1H, m), 4.11-4.18 (1H, m), 4.26 (2H, d, J = 6.5 Hz), 4.31 (2H, d, |
| | J = 5.4 Hz), 6.50 (2H, s), 6.56 (1H, d, J = 2.4 Hz), 6.60 (1H, d, J = 6.0 Hz), 6.76-6.80 |
| | (1H, m), 6.84 (1H, d, J = 8.5 Hz), 6.89 (1H, dd, J = 9.0, 2.3 Hz), 7.54 (1H, d, J = 6.0 |
| | Hz), 7.66 (1H, d, J = 1.5 Hz), 7.74 (1H, dd, J = 8.4, 2.5 Hz), 7.88 (1H, d, J = 9.0 Hz), |
| | 8.20 (1H, d, J = 2.4 Hz) |
| 1029 | 1.66-1.78 (1H, m), 2.10-2.16 (1H, m), 2.32-2.42 (1H, m), 2.47-2.54 (1H, m, |
| | obscured by DMSO), 2.89-2.98 (1H, m), 3.85-3.94 (1H, m), 4.05-4.12 (1H, m), |
| | 4.24 (2H, d, J = 6.6 Hz), 4.40 (2H, d, J = 5.7 Hz), 6.58 (1H, d, J = 7.8 Hz), 6.66 (2H, s), |
| | 6.71 (1H, t, J = 6.0 Hz), 6.80 (1H, d, J = 8.5 Hz), 6.86 (1H, d, J = 1.3 Hz), 7.03 (1H, d, |
| | J = 1.3 Hz), 7.17 (1H, t, J = 8.0 Hz), 7.20 (1H, d, J = 6.2 Hz), 7.35 (1H, d, J = 8.3 Hz), |
| | 7.70-7.75 (2H, m), 8.19 (1H, d, J = 2.4 Hz) |
| 1030 | 1.65-1.78 (1H, m), 2.10-2.16 (1H, m), 2.31-2.42 (1H, m), 2.46-2.54 (1H, m, |
| | obscured by DMSO), 2.88-2.97 (1H, m), 3.83-3.94 (1H, m), 4.04-4.12 (1H, m), |
| | 4.24 (2H, d, J = 6.6 Hz), 4.40 (2H, d, J = 5.7 Hz), 6.57 (1H, d, J = 7.8 Hz), 6.61 (2H, s), |
| | 6.69 (1H, t, J = 6.0 Hz), 6.80 (1H, d, J = 8.5 Hz), 6.84 (1H, d, J = 1.3 Hz), 7.02 (1H, d, |
| | J = 1.3 Hz), 7.16 (1H, t, J = 8.0 Hz), 7.19 (1H, d, J = 6.2 Hz), 7.34 (1H, d, J = 8.3 Hz), |
| | 7.70-7.75 (2H, m), 8.19 (1H, d, J = 2.4 Hz) |
| 1031 | 2.15 (2H, m), 4.10 (2H, t, J = 7.0 Hz), 4.16 (2H, t, J = 6.3 Hz), 4.29 (2H, d, J = 5.7 Hz), |
| | 6.33 (2H, s), 6.53 (1H, d, J = 2.3 Hz), 6.57 (1H, d, J = 5.9 Hz), 6.69 (1H, t, J = 5.9 Hz), |
| | 6.80 (1H, d, J = 8.5 Hz), 6.85-6.89 (2H, m), 7.18 (1H, d, J = 1.3 Hz), 7.55 (1H, d, J = |
| | 5.8 Hz), 7.61 (1H, d, J = 1.2 Hz), 7.72 (1H, dd, J = 8.5, 2.4 Hz), 7.85 (1H, d, J = 9.0 Hz), |
| | 8.17 (1H, d, J = 2.4 Hz) |
| 1032 | 1.13-1.23 (2H, m), 1.31-1.42 (1H, m), 1.56-1.67 (4H, m), 1.77-1.86 (2H, m), |
| | 2.13 (3H, s), 2.69-2.78 (2H, m), 4.26 (2H, t, J = 6.6 Hz), 4.29 (2H, d, J = 5.8 Hz), 6.38 |
| | (2H, s), 6.53 (1H, d, J = 2.3 Hz), 6.58 (1H, d, J = 5.9 Hz), 6.70 (1H, t, J = 5.9 Hz), 6.77 |
| | (1H, d, J = 8.5 Hz), 6.87 (1H, dd, J = 9.0, 2.4 Hz), 7.54 (1H, d, J = 5.9 Hz), 7.70 (1H, |
| | dd, J = 8.5, 2.5 Hz), 7.86 (1H, d, J = 9.0 Hz), 8.18 (1H, d, J = 2.4 Hz) |
| 1033 | 1.10-1.20 (2H, m), 1.41-1.52 (1H, m), 1.61-1.69 (2H, m), 1.73-1.82 (2H, m), |
| | 2.12 (3H, s), 2.67-2.77 (2H, m), 3.06-3.12 (2H, m), 4.12 (2H, d, J = 5.6 Hz), 6.34 |
| | (2H, s), 6.42-6.48 (2H, m), 6.50 (1H, t, J = 5.6 Hz), 6.53 (1H, d, J = 2.3 Hz), 6.58 (1H, |
| | d, J = 5.8 Hz), 6.86 (1H, dd, J = 9.1, 2.3 Hz), 7.37 (1H, dd, J = 8.6, 2.4 Hz), 7.54 (1H, d, |
| | J = 5.9 Hz), 7.84 (1H, d, J = 9.1 Hz), 7.98 (1H, d, J = 2.4 Hz) |
| 1034 | 2.32 (4H, s), 3.43 (2H, s), 3.55 (4H, t, J = 4.5 Hz), 4.46 (2H, d, J = 5.7 Hz), 6.46 (1H, d, |
| | J = 7.7 Hz), 6.50 (2H, s), 6.66 (1H, t, J = 5.8 Hz), 7.04 (1H, d, J = 7.9 Hz), 7.11-7.20 |
| | (3H, m), 7.26-7.33 (2H, m), 7.75 (1H, d, J = 6.0 Hz) |
| 1035 | 2.10 (2H, dt, J = 7.7, 6.6 Hz), 2.73 (2H, t, J = 7.5 Hz), 3.53 (3H, s), 4.32 (2H, t, J = 6.5 |
| | Hz), 4.39 (2H, d, J = 5.8 Hz), 6.51 (2H, s), 6.60 (2H, t, J = 6.2 Hz), 6.72 (1H, d, J = 1.2 |
| | Hz), 6.98 (1H, d, J = 1.2 Hz), 7.13-7.23 (2H, m), 7.36 (1H, d, J = 8.3 Hz), 7.74 (1H, d, |
| | J = 6.0 Hz), 8.59 (2H, s) |
| 1036 | 0.78 (6H, d, J = 6.2 Hz), 2.11 (1H, p, J = 6.2 Hz), 3.07 (4H, s), 3.14 (4H, s), 3.45 (2H, |
| | s), 4.45 (2H, d, J = 5.8 Hz), 6.44 (1H, d, J = 7.7 Hz), 6.50 (2H, s), 6.66 (1H, t, J = 6.0 |
| | Hz), 6.97 (1H, dd, J = 7.9, 1.6 Hz), 7.02-7.07 (1H, t, J = 8.0 Hz), 7.17-7.21 (1H, m), |
| | 7.24 (1H, t, J = 7.9 Hz), 7.32 (1H, d, J = 8.3 Hz), 7.74 (1H, d, J = 6.0 Hz) |
| 1037 | 0.85 (6H, d, J = 6.2, 3.1 Hz), 2.33-2.42 (2H, m), 3.19 (4H, s), 3.30 (4H, s), 3.49 (2H, |
| | s), 4.38 (2H, d, J = 5.7 Hz), 6.59 (1H, dd, J = 23.7, 2.3 Hz), 6.64 (1H, d, J = 6.2 Hz), |
| | 6.95 (1H, dd, J = 9.1, 2.4 Hz), 6.98-7.10 (3H, m), 7.33 (1H, dt, J = 56.3, 7.9 Hz), 7.46 |
| | (1H, d, J = 6.3 Hz), 7.52 (1H, s), 7.95 (1H, d, J = 9.1 Hz), 8.28 (3H, s) |
| 1038 | 1.88 (2H, p, J = 7.2 Hz), 2.64 (2H, t, J = 7.6 Hz), 3.28-3.34 (7H, m, should be 2H, in |
| | water peak), 3.51 (3H, s), 4.17 (2H, d, J = 5.3 Hz), 6.57 (2H, s), 6.71 (1H, d, J = 1.2 |
| | Hz), 6.72 (1H, d, J = 2.3 Hz), 6.90 (1H, t, J = 5.4 Hz), 6.94 (1H, dd, J = 9.0, 2.3 Hz), |
| | 6.97 (1H, d, J = 1.2 Hz), 7.24 (1H, t, J = 5.7 Hz), 7.67 (1H, s), 7.92 (1H, d, J = 9.1 Hz), |
| | 8.29 (2H, d, J = 16.3 Hz) |
| 1039 | 1.67-1.79 (2H, m), 1.77-1.88 (2H, m), 2.43-2.48 (2H, m), 3.25 (2H, t, J = 6.8 Hz), |
| | 3.29-3.37 (2H, m), 3.45 (2H, q, J = 6.8 Hz), 4.23 (2H, d, J = 5.6 Hz), 6.44-6.51 (3H, |
| | m), 6.61 (1H, d, J = 7.7 Hz), 6.91 (1H, t, J = 5.9 Hz), 7.11-7.22 (2H, m), 7.34 (1H, d, |
| | J = 8.3 Hz), 7.72 (1H, d, J = 6.1 Hz), 8.30 (2H, s) |
| 1040 | 1.65-1.79 (2H, m), 1.77-1.88 (2H, m), 2.43-2.49 (2H, m), 3.26 (2H, t, J = 6.8 Hz), |
| | 3.34-3.38 (2H, m), 3.46 (2H, q, J = 6.8 Hz), 4.18 (2H, d, J = 5.5 Hz), 6.56 (2H, s), |
| | 6.72 (1H, d, J = 2.3 Hz), 6.87-6.98 (2H, m), 6.99 (1H, t, J = 5.9 Hz), 7.67 (1H, s), 7.92 |
| | (1H, d, J = 9.1 Hz), 8.32 (2H, s) |
| 1041 | 1.59-1.73 (1H, m), 1.96-2.12 (1H, m), 2.45 (1H, br s), 2.64-2.77 (1H, m), 2.77- |
| | 2.88 (1H, m), 3.72 (1H, dd, J = 12.3, 10.1 Hz), 4.16 (1H, dd, J = 12.3, 5.2 Hz), 4.22 |
| | (1H, dd, J = 10.7, 7.4 Hz), 4.32 (1H, d, J = 6.0 Hz), 4.34 (2H, d, J = 5.6 Hz), 6.55 (2H, |
| | s), 6.71 (1H, d, J = 2.3 Hz), 6.79 (1H, d, J = 1.2 Hz), 6.82-6.86 (1H, m), 6.95 (1H, dd, |
| | J = 9.1, 2.4 Hz), 6.97-7.02 (1H, m), 7.06 (1H, t, J = 5.8 Hz), 7.65 (1H, s), 7.74 (1H, |
| | dd, J = 8.5, 2.5 Hz), 7.92 (1H, d, J = 9.0 Hz), 8.20 (1H, d, J = 2.4 Hz) |
| 1042 | 1H NMR (CDCl3, 400 MHz) δ 1.81-1.93 (1H, m), 2.35 (1H, d, J = 14.5 Hz), 2.46- |
| | 2.59 (1H, m), 2.65 (1H, dd, J = 16.3, 10.9 Hz), 3.15 (1H, dd), 3.94 (1H, td, J = 11.8, |
| | 4.8 Hz), 4.07-4.17 (1H, m), 4.33 (1H, dd, J = 10.7, 7.6 Hz), 4.42-4.51 (3H, m), 4.58 |
| | (1H, d, J = 5.5 Hz), 5.20 (2H, s), 6.73 (1H, d, J = 7.7 Hz), 6.81 (1H, d, J = 1.3 Hz), 6.95 |
| | (1H, dd, J = 6.2, 1.0 Hz), 6.99 (1H, d, J = 1.4 Hz), 7.21 (1H, d, J = 8.4 Hz), 7.36 (1H, t, |
| | J = 8.0 Hz), 7.93 (1H, d, J = 6.2 Hz), 8.58 (2H, s) |
| 1043 | 1H NMR (CDCl3, 400 MHz) δ 1.68-1.83 (1H, m), 2.16 (1H, d, J = 13.5 Hz), 2.31 (1H, |
| | s), 2.53 (1H, dd, J = 16.5, 10.8 Hz), 3.11 (1H, dd, J = 16.4, 5.0 Hz), 3.45-3.62 (2H, |
| | m), 3.89 (1H, td, J = 11.8, 4.8 Hz), 4.05-4.14 (1H, m), 4.30 (2H, d, J = 5.2 Hz), 4.42 |
| | (1H, s), 4.91 (2H, s), 5.33 (1H, t, J = 6.3 Hz), 6.79 (1H, d), 6.87 (1H, dd, J = 8.9, 2.4 |
| | Hz), 6.98 (1H, d, J = 1.4 Hz), 7.03 (1H, d, J = 2.4 Hz), 7.60 (1H, d, J = 8.9 Hz), 7.88 |
| | (1H, s), 8.34 (2H, s) |
| 1044 | 1.54-1.69 (1H, m), 2.05 (1H, d, J = 13.5 Hz), 2.11-2.19 (1H, m), 2.37 (1H, dd, J = |
| | 16.4, 10.6 Hz), 2.88 (1H, dd, J = 16.4, 5.1, 1.5 Hz), 3.22-3.31 (2H, m), 3.81 (1H, td, |
| | J = 12.3, 11.8, 4.7 Hz), 4.00-4.10 (1H, m), 4.17 (2H, d, J = 5.4 Hz), 6.50 (1H, d, J = 8.5, |
| | 0.7 Hz), 6.54 (2H, s), 6.67 (1H, t, J = 5.8 Hz), 6.71 (1H, d, J = 2.3 Hz), 6.78 (1H, d, J = |
| | 1.2 Hz), 6.86-6.98 (3H, m), 7.41 (1H, dd, J = 8.6, 2.4 Hz), 7.65 (1H, s), 7.90 (1H, d, |
| | J = 9.1 Hz), 8.01 (1H, d, J = 2.3 Hz) |
| 1049 | 2.18-2.26 (2H, m), 2.92 (1H, dd, J = 16.8, 5.1 Hz), 3.15 (1H, dd, J = 16.8, 4.6 Hz), |
| | 3.94-4.08 (2H, m), 4.39 (2H, d, J = 5.8 Hz), 5.47-5.58 (1H, m), 6.49 (2H, s), 6.55 |
| | (1H, d, J = 7.6 Hz), 6.66 (1H, t, J = 6.0 Hz), 6.74 (1H, d, J = 8.5 Hz), 6.82 (1H, d, J = 1.2 |
| | Hz), 7.02 (1H, d, J = 1.2 Hz), 7.12-7.19 (2H, m), 7.32 (1H, d, J = 8.3 Hz), 7.70 (1H, |
| | dd, J = 8.5, 2.5 Hz), 7.73 (1H, d, J = 6.0 Hz), 8.21 (1H, d, J = 2.4 Hz) |
| 1050 | 2.20-2.27 (2H, m), 2.93 (1H, dd, J = 16.8, 5.0 Hz), 3.16 (1H, dd, J = 16.9, 4.6 Hz), |
| | 3.96-4.10 (2H, m), 4.34 (2H, d, J = 5.7 Hz), 5.50-5.59 (1H, m), 6.55 (2H, s), 6.72 |
| | (1H, d, J = 2.3 Hz), 6.79 (1H, dd, J = 8.5, 0.7 Hz), 6.82 (1H, d, J = 1.3 Hz), 6.95 (1H, |
| | dd, J = 9.1, 2.4 Hz), 7.03 (1H, d, J = 1.3 Hz), 7.06 (1H, d, J = 5.8 Hz), 7.66 (1H, s), 7.73 |
| | (1H, dd, J = 8.5, 2.5 Hz), 7.92 (1H, d, J = 9.0 Hz), 8.23 (1H, d, J = 2.1 Hz) |
| 1052 | 1.65-1.81 (1H, m), 2.05-2.19 (1H, m), 2.33-2.44 (1H, m), 2.86-2.97 (1H, m), |
| | 3.88 (1H, td, J = 11.9, 4.7 Hz), 4.02-4.14 (1H, m), 4.30 (2H, dd, J = 6.5, 1.5 Hz), 4.37 |
| | (2H, d, J = 5.6 Hz), 6.58 (2H, br s), 6.73 (1H, d, J = 2.3 Hz), 6.80 (1H, d, J = 1.3 Hz), |
| | 6.96 (1H, dd, J = 9.1, 2.4 Hz), 6.98 (1H, d, J = 1.3 Hz), 7.04 (1H, t, J = 5.7 Hz), 7.67 |
| | (1H, s), 7.94 (1H, d, J = 9.0 Hz), 8.65 (2H, s). One aliphatic proton obscured by |
| | water peak |
| 1096 | 1.14 (2H, qd, J = 12.0, 3.8 Hz), 1.39-1.51 (1H, m), 1.60-1.69 (2H, m), 1.76 (2H, td, |
| | J = 11.7, 2.5 Hz), 2.11 (3H, s), 2.72 (2H, d, J = 11.6 Hz), 3.08 (2H, t, J = 6.3 Hz), 3.97 |
| | (2H, d, J = 5.7 Hz), 5.26 (2H, s), 5.57 (1H, d, J = 2.0 Hz), 5.84 (1H, dd, J = 5.8, 2.1 Hz), |
| | 6.37 (1H, t, J = 5.7 Hz), 6.40-6.48 (2H, m), 7.28 (1H, dd, J = 8.6, 2.4 Hz), 7.42 (1H, d, |
| | J = 5.8 Hz), 7.89 (1H, d, J = 2.4 Hz) |
| 1102 | 1.04-1.23 (2H, m), 1.36-1.53 (1H, m), 1.64 (2H, d, J = 12.8 Hz), 1.77 (2H, t, J = 11.5 |
| | Hz), 2.12 (3H, s), 2.57 (3H, s), 2.72 (2H, d, J = 11.4 Hz), 3.01-3.13 (2H, m), 4.14 (2H, |
| | d, J = 5.6 Hz), 6.36-6.51 (2H, m), 6.53 (1H, d, J = 2.2 Hz), 6.76 (1H, t, J = 5.6 Hz), |
| | 6.90 (1H, dd, J = 2.2, 1.1 Hz), 7.31-7.40 (2H, m), 7.99 (1H, d, J = 2.5 Hz), 8.18 (1H, |
| | d, J = 5.7 Hz), 8.99 (1H, s) |
| 1103 | 1.14 (2H, qd, J = 12.0, 3.8 Hz), 1.38-1.52 (1H, m), 1.64 (2H, d, J = 12.7 Hz), 1.76 (2H, |
| | td, J = 11.6, 2.5 Hz), 2.11 (3H, s), 2.72 (2H, d, J = 11.3 Hz), 3.09 (2H, t, J = 6.3 Hz), 3.89 |
| | (3H, s), 4.15 (2H, d, J = 5.4 Hz), 6.29 (1H, d, J = 1.8 Hz), 6.45 (1H, d, J = 8.5 Hz), 6.47 |
| | (1H, t, J = 5.8 Hz), 6.54 (1H, d, J = 1.9 Hz), 6.78 (1H, t, J = 5.5 Hz), 7.30 (1H, dd, J = 5.9, |
| | 0.9 Hz), 7.37 (1H, dd, J = 8.6, 2.4 Hz), 7.99 (1H, d, J = 2.4 Hz), 8.15 (1H, d, J = 5.8 Hz), |
| | 9.01 (1H, s) |
| 1116 | (500 MHz, Methanol-d4) 7.93 (s, 1H), 7.73-7.67 (m, 2H), 7.57 (ddd, J = 8.3, 2.2, 0.9 |
| | Hz, 1H), 7.49 (dd, J = 8.7, 2.4 Hz, 1H), 7.43-7.36 (m, 2H), 7.22 (ddd, J = 8.0, 2.0, 0.9 |
| | Hz, 1H), 6.52 (d, J = 8.6 Hz, 1H), 4.03 (s, 2H), 3.16 (d, J = 6.9 Hz, 2H), 2.87 (app d, J = |
| | 11.3 Hz, 2H), 2.25 (s, 3H), 1.99 (app t, J = 11.8 Hz, 2H), 1.80 (app d, J = 13.2 Hz, 2H), |
| | 1.68-1.54 (m, 1H), 1.29 (app qd, J = 12.4, 3.8 Hz, 2H) |
| 1118 | 2.04-2.12 (1H, m), 2.13 (3H, s), 2.37-2.44 (1H, m), 2.54-2.75 (2H, m), 4.18 (2H, d, |
| | J = 5.4 Hz), 5.11-5.17 (1H, m), 6.53 (1H, d, J = 8.6 Hz), 6.58 (2H, s), 6.70 (1H, s), 6.73 |
| | (1H, d, J = 2.2 Hz), 6.91-6.96 (2H, m), 7.40 (1H, dd, J = 8.6, 2.4 Hz), 7.66 (1H, s), |
| | 7.92 (1H, d, J= 9.1 Hz), 8.04 (1H, d, J = 1.8 Hz), 12.08 (1H, s) |
| 1119 | 2.06-2.11 (1H, m), 2.13 (3H, s), 2.33-2.44 (1H, m), 2.54-2.60 (1H, m), 2.67-2.77 |
| | (1H, m), 4.25 (2H, d, J = 5.6 Hz), 5.10-5.15 (1H, m), 6.47-6.63 (6H, m), 7.14-7.17 |
| | (2H, m), 7.31 (1H, d, J = 8.3 Hz), 7.39 (1H, dd, J = 8.5, 2.3 Hz), 7.72 (1H, d, J = 6.1 |
| | Hz), 8.02 (1H, d, J = 1.9 Hz), 8.30 (1H, s) |
| 1128 | 3.94-4.02 (2H, m), 4.02-4.08 (2H, m), 4.34 (2H, d, J = 5.8 Hz), 4.63 (2H, s), 6.50 |
| | (2H, s), 6.54 (1H, d, J = 7.7 Hz), 6.63 (1H, t, J = 6.0 Hz), 6.87 (1H, d, J = 1.2 Hz), 6.96 |
| | (1H, d, J = 8.7 Hz), 7.08 (1H, d, J = 1.2 Hz), 7.14 (1H, t, J = 8.1 Hz), 7.17 (1H, d, J = 6.1 |
| | Hz), 7.30 (1H, d, J = 8.3 Hz), 7.61 (1H, dd, J = 8.7, 2.4 Hz), 7.73 (1H, d, J = 6.1 Hz), |
| | 8.21 (1H, d, J = 2.3 Hz). |
| 1129 | 1H NMR (DMSO, 400 MHz) ? 3.98-4.03 (2H, m), 4.03-4.08 (2H, m), 4.27 (2H, d, |
| | J = 5.6 Hz), 4.66 (2H, s), 6.54 (2H, s), 6.72 (1H, d, J = 2.3 Hz), 6.87 (1H, d, J = 1.3 Hz), |
| | 6.94 (1H, dd, J = 9.1, 2.4 Hz), 6.97-7.03 (2H, m), 7.08 (1H, d, J = 1.2 Hz), 7.63 (1H, |
| | dd, J = 8.9, 2.6 Hz), 7.65 (1H, s), 7.91 (1H, d, J = 9.1 Hz), 8.22 (1H, d, J = 2.4 Hz) |
| 1130 | 1.12 (6H, d, J = 3.7 Hz), 1.77-1.94 (2H, m), 2.24 (1H, p, J = 8.8 Hz), 3.13-3.26 (2H, |
| | m), 3.39 (1H, dd, J = 10.2, 8.3 Hz), 3.47-3.54 (1H, m), 4.28 (2H, d, J = 5.7 Hz), 4.32 |
| | (1H, s), 6.36 (1H, d, J = 8.6 Hz), 6.48 (2H, s), 6.54 (2H, d, J = 7.4 Hz), 7.12 (1H, d, J = |
| | 8.0 Hz), 7.16 (1H, d, J = 6.2 Hz), 7.30 (1H, d, J = 8.4 Hz), 7.48 (1H, dd, J = 8.6, 2.4 Hz), |
| | 7.72 (1H, d, J = 6.1 Hz), 8.08 (1H, d, J = 2.3 Hz). |
| 1131 | 1.11 (3H, s), 1.12 (3H, s), 1.75-1.94 (3H, m), 2.24 (1H, p, J = 8.7 Hz), 3.13-3.26 |
| | (2H, m), 3.50 (2H, td, J = 8.8, 8.2, 4.7 Hz), 4.28 (2H, d, J = 5.7 Hz), 6.36 (1H, d, J = 8.6 |
| | Hz), 6.49 (2H, s), 6.53 (1H, s), 6.56 (1H, q, J = 4.9, 4.0 Hz), 7.12 (1H, d, J = 8.0 Hz), |
| | 7.16 (1H, d, J = 5.8 Hz), 7.30 (1H, d, J = 8.3 Hz), 7.48 (1H, dd, J = 8.6, 2.4 Hz), 7.72 |
| | (1H, d, J = 6.1 Hz), 8.08 (1H, d, J = 2.3 Hz). |
| 1133 | 3.64 (2H, d, J = 6.7 Hz), 3.90 (1H, dd, J = 13.5, 4.2 Hz), 4.04 (1H, dd, J = 13.6, 5.4 Hz), |
| | 4.13-4.22 (1H, m), 4.34 (2H, d, J = 5.8 Hz), 4.60 (2H, q, J = 16.2 Hz), 5.18 (1H, s), |
| | 6.50 (2H, s), 6.55 (1H, d, J = 7.6 Hz), 6.63 (1H, t, J = 5.7 Hz), 6.80-6.90 (1H, m), 6.95 |
| | (1H, d, J = 8.7 Hz), 7.08-7.18 (2H, m), 7.19-7.22 (1H, m), 7.30 (1H, d, J = 8.3 Hz), |
| | 7.61 (1H, dd, J = 8.8, 2.4 Hz), 7.73 (1H, d, J = 6.0 Hz), 8.21 (1H, s) |
| 1135 | 0.85 (3H, s), 1.23 (3H, s), 3.60 (1H, dd, J = 14.0, 4.5 Hz), 4.07 (1H, t, J = 4.3 Hz), 4.34 |
| | (2H, d, J = 5.8 Hz), 4.46 (1H, s), 4.58 (1H, s), 4.62 (1H, d, J = 7.0 Hz), 4.91 (1H, s), |
| | 6.50 (2H, s), 6.56 (1H, d, J = 7.7 Hz), 6.62 (1H, t, J = 5.8 Hz), 6.86 (1H, t, J = 1.3 Hz), |
| | 7.11-7.20 (2H, m), 7.25 (1H, d, J = 1.3 Hz), 7.31 (1H, d, J = 8.3 Hz), 7.61 (1H, dd, J = |
| | 8.7, 2.6 Hz), 7.73 (1H, d, J = 6.1 Hz), 8.21 (1H, d, J = 2.4 Hz) |
| 1137 | 2.83 (1H, s), 3.37-3.40 (1H, m), 3.69 (1H, t, J = 11.2 Hz), 3.82 (1H, d, J = 16.4 Hz), |
| | 3.93 (1H, d, J = 16.1 Hz), 4.07 (1H, dd, J = 12.0, 4.1 Hz), 4.24 (1H, dd, J = 10.7, 6.0 |
| | Hz), 4.34 (1H, dd, J = 10.7, 5.9 Hz), 4.39 (2H, d, J = 6.0 Hz), 6.43-6.61 (3H, m), 6.68 |
| | (1H, t, J = 6.0 Hz), 6.74-6.85 (2H, m), 7.01 (1H, s), 7.10-7.20 (2H, m), 7.32 (1H, d, |
| | J = 8.3 Hz), 7.64-7.79 (2H, m), 8.19 (1H, s) |
| 1140 | 2.93-3.01 (1H, m), 3.19-3.25 (1H, m), 3.86-3.94 (2H, m), 4.19-4.32 (2H, m), |
| | 4.39 (2H, d, J = 5.8 Hz), 4.78 (1H, d, J = 9.2 Hz), 6.49-6.59 (3H, m), 6.68 (1H, t, J = |
| | 5.9 Hz), 6.77 (1H, d, J = 8.4 Hz), 6.83 (1H, s), 7.05 (1H, s), 7.10-7.20 (2H, m), 7.32 |
| | (1H, d, J = 8.2 Hz), 7.67-7.77 (2H, m), 8.13-8.22 (1H, m). 1H hidden under water |
| 1150 | (CD3CN, 400 MHz) 1.49 (3H, d, J = 6.7 Hz), 3.50 (1H, ddd, J = 14.2, 11.2, 4.2 Hz), |
| | 4.00 (1H, td, J = 12.1, 11.6, 4.2 Hz), 4.04-4.11 (1H, m), 4.40 (2H, d, J = 5.6 Hz), 4.68- |
| | 4.77 (1H, m), 5.37 (1H, s), 5.46 (1H, s), 5.50 (2H, t, J = 6.7 Hz), 6.75 (1H, d, J = 7.7 |
| | Hz), 6.83 (1H, d, J = 8.7 Hz), 6.93 (2H, t, J = 1.0 Hz), 7.07 (1H, dd, J = 6.1, 1.0 Hz), |
| | 7.21 (1H, dt, J = 8.3, 1.0 Hz), 7.31 (1H, t, J = 8.0 Hz), 7.63 (1H, dd, J = 8.8, 2.5 Hz), |
| | 7.85 (1H, d, J = 6.1 Hz), 8.23 (1H, dd, J = 2.4, 0.8 Hz) |
| 1156 | 4.04 (2H, dd, J = 6.2, 4.6 Hz), 4.14 (2H, t, J = 5.4 Hz), 4.33 (2H, d, J = 5.8 Hz), 4.72 |
| | (2H, s), 6.12 (1H, d, J = 1.9 Hz), 6.51 (2H, s), 6.54 (1H, d, J = 7.6 Hz), 6.62 (1H, t, J = |
| | 5.9 Hz), 6.96 (1H, d, J = 8.7 Hz), 7.14 (1H, t, J = 8.0 Hz), 7.17 (1H, d, J = 6.1 Hz), 7.31 |
| | (1H, d, J = 8.3 Hz), 7.40 (1H, d, J = 1.9 Hz), 7.62 (1H, dd, J = 8.7, 2.4 Hz), 7.73 (1H, d, |
| | J = 6.0 Hz), 8.21 (1H, d, J = 2.4 Hz) |
| 1157 | 4.06 (2H, dd, J = 6.3, 4.5 Hz), 4.15 (2H, t, J = 5.4 Hz), 4.27 (2H, d, J = 5.6 Hz), 4.75 |
| | (2H, s), 6.13 (1H, d, J = 1.8 Hz), 6.60 (2H, s), 6.72 (1H, d, J = 2.3 Hz), 6.94 (1H, dd, J = |
| | 9.1, 2.3 Hz), 6.98-7.05 (2H, m), 7.41 (1H, d, J = 1.8 Hz), 7.59-7.65 (1H, m), 7.66 |
| | (1H, s), 7.92 (1H, d, J = 9.1 Hz), 8.22 (1H, d, J = 2.4 Hz) |
| 1163 | 1.35 (6H, s), 3.97 (4H, s), 4.33 (2H, d, J = 5.8 Hz), 4.61 (2H, d, J = 2.2 Hz), 6.50 (2H, |
| | s), 6.54 (1H, d, J = 7.7 Hz), 6.63 (1H, t, J = 6.0 Hz), 6.82 (1H, s), 6.95 (1H, d, J = 8.7 |
| | Hz), 7.08-7.21 (2H, m), 7.30 (1H, d, J = 8.3 Hz), 7.61 (1H, dd, J = 8.7, 2.4 Hz), 7.72 |
| | (1H, d, J = 6.1 Hz), 8.20 (1H, d, J = 2.4 Hz). OH shift hidden under solvent |
| 1167 | 1.45 (6H, s), 3.94 (2H, t, J = 5.5 Hz), 4.24 (2H, t, J = 5.3 Hz), 4.34 (2H, d, J = 5.9 Hz), |
| | 4.60 (2H, s), 5.06 (1H, s), 6.48 (2H, s), 6.55 (1H, d, J = 7.7 Hz), 6.61 (1H, t, J = 6.0 Hz), |
| | 6.65 (1H, s), 6.94 (1H, d, J = 8.7 Hz), 7.04-7.20 (2H, m), 7.30 (1H, d, J = 8.3 Hz), |
| | 7.61 (1H, dd, J = 8.7, 2.5 Hz), 7.73 (1H, d, J = 6.1 Hz), 8.21 (1H, d, J = 2.4 Hz) |
| 1175 | 1.38-1.43 (2H, m), 1.49-1.56 (1H, m), 2.17-2.22 (5H, m), 2.89 (2H, d, J = 8.7 Hz), |
| | 4.03 (2H, d, J = 7.4 Hz), 4.28 (2H, d, J = 5.8 Hz), 6.29 (2H, s), 6.53 (1H, d, J = 2.4 Hz), |
| | 6.57 (1H, d, J = 5.9 Hz), 6.66 (1H, t, J = 5.9 Hz), 6.78 (1H, d, J = 8.5 Hz), 6.86 (1H, dd, |
| | J = 9.0, 2.4 Hz), 7.55 (1H, d, J = 5.8 Hz), 7.70 (1H, dd, J = 8.5, 2.5 Hz), 7.85 (1H, d, J = |
| | 9.1 Hz), 8.16 (1H, d, J = 2.4 Hz) |
| 1176 | 1.12 (3H, s), 1.13 (3H, s), 1.81-1.94 (2H, m), 2.24 (1H, q, J = 8.8 Hz), 3.13-3.25 |
| | (2H, m), 3.52 (2H, t, J = 9.2 Hz), 4.21 (2H, d, J = 5.6 Hz), 4.32 (1H, s), 6.40 (1H, d, J = |
| | 8.6 Hz), 6.53 (2H, s), 6.72 (1H, d, J = 2.3 Hz), 6.89-6.98 (2H, m), 7.50 (1H, dd, J = |
| | 8.7, 2.4 Hz), 7.65 (1H, s), 7.90 (1H, d, J = 9.1 Hz), 8.09 (1H, d, J = 2.4 Hz) |
| 1177 | 1.14 (qd, J = 12.0, 3.8 Hz, 2H), 1.38-1.51 (m, 1H), 1.61-1.67 (m, 2H), 1.72-1.81 |
| | (m, 2H), 2.11 (s, 3H), 2.69-2.73 (m, 2H), 3.08 (t, J = 6.3 Hz, 2H), 3.82 (s, 3H), 4.13 |
| | (d, J = 5.6 Hz, 2H), 6.42-6.50 (m, 2H), 6.61 (t, J = 5.6 Hz, 1H), 6.93 (dd, J = 9.0, 2.7 |
| | Hz, 1H), 6.97 (d, J = 2.7 Hz, 1H), 7.02 (d, J = 2.8 Hz, 1H), 7.38 (dd, J = 8.6, 2.4 Hz, |
| | 1H), 7.64 (d, J = 9.0 Hz, 1H), 8.01 (d, J = 2.4 Hz, 1H), 8.31 (d, J = 2.7 Hz, 1H) |
| 1178 | 1.14 (qd, J = 12.0, 3.9 Hz, 2H), 1.40-1.50 (m, 1H), 1.62-1.68 (m, 2H), 1.76 (td, J = |
| | 11.6, 2.4 Hz, 2H), 2.11 (s, 3H), 2.68-2.75 (m, 2H), 3.08 (t, J = 6.3 Hz, 2H), 3.89 (s, |
| | 3H), 4.11 (d, J = 5.5 Hz, 2H), 6.24 (t, J = 5.6 Hz, 1H), 6.41-6.46 (m, 2H), 6.78 (d, J = |
| | 7.6 Hz, 1H), 6.97-7.06 (m, 3H), 7.22 (d, J = 8.1 Hz, 1H), 7.37 (dd, J = 8.6, 2.4 Hz, |
| | 1H), 7.55 (d, J = 8.6 Hz, 1H), 7.97 (d, J = 2.3 Hz, 1H) |
| 1180 | 1.07-1.21 (m, 2H), 1.39-1.52 (m, 1H), 1.65 (d, J = 12.7 Hz, 2H), 1.77 (t, J = 11.5 |
| | Hz, 2H), 2.12 (s, 3H), 2.72 (d, J = 11.3 Hz, 2H), 3.08 (t, J = 6.3 Hz, 2H), 3.78 (s, 3H), |
| | 4.08 (d, J = 5.6 Hz, 2H), 6.00 (t, J = 5.7 Hz, 1H), 6.42 (d, J = 2.9 Hz, 1H), 6.44 (s, 1H), |
| | 6.73 (d, J = 2.3 Hz, 1H), 6.96 (t, J = 3.0 Hz, 1H), 6.98 (t, J = 2.9 Hz, 1H), 7.08 (d, J = |
| | 2.6 Hz, 1H), 7.38 (dd, J = 8.5, 2.4 Hz, 1H), 7.48 (d, J = 8.9 Hz, 1H), 7.51 (d, J = 8.8 Hz, |
| | 1H), 7.99 (d, J = 2.3 Hz, 1H) |
| 1181 | 3.00 (2H, t, J = 7.3 Hz), 3.56 (2H, q, J = 6.8 Hz), 4.25 (2H, d, J = 5.6 Hz), 6.43 (1H, d, |
| | J = 8.6 Hz), 6.48 (2H, s), 6.51-6.57 (3H, m), 6.96 (1H, d, J = 1.5 Hz), 7.13-7.20 (2H, |
| | m), 7.30 (1H, d, J = 8.3 Hz), 7.40 (1H, dd, J = 8.6, 2.3 Hz), 7.45 (1H, d, J = 1.5 Hz), |
| | 7.72 (1H, d, J = 6.1 Hz), 7.82 (1H, t, J = 59.3 Hz), 8.03 (1H, d, J = 2.1 Hz) |
| 1182 | 3.01 (2H, t, J = 7.2 Hz), 3.55-3.60 (2H, m), 4.19 (2H, d, J = 5.5 Hz), 6.47 (1H, d, J = |
| | 8.5 Hz), 6.58 (2H, s), 6.62 (1H, t, J = 5.9 Hz), 6.72 (1H, d, J = 2.2 Hz), 6.90-6.97 (3H, |
| | m), 7.42 (1H, dd, J = 8.5, 2.4 Hz), 7.45 (1H, d, J = 1.6 Hz), 7.65 (1H, s), 7.91 (1H, t, J = |
| | 59.3 Hz), 7.91 (1H, d, J = 9.1 Hz), 8.05 (1H, d, J = 2.1 Hz) |
| 1183 | 1.08-1.20 (m, 2H), 1.45 (ddp, J = 10.9, 7.0, 3.7, 3.2 Hz, 1H), 1.61-1.68 (m, 2H), |
| | 1.76 (td, J = 11.6, 2.5 Hz, 2H), 2.11 (s, 3H), 2.71 (dt, J = 11.6, 3.3 Hz, 2H), 3.08 (t, J = |
| | 6.3 Hz, 2H), 4.14 (d, J = 5.6 Hz, 2H), 6.42-6.50 (m, 2H), 6.63 (t, J = 5.6 Hz, 1H), 7.06 |
| | (d, J = 2.7 Hz, 1H), 7.31 (ddd, J = 8.3, 6.8, 1.5 Hz, 1H), 7.38 (ddd, J = 13.5, 8.3, 1.9 |
| | Hz, 2H), 7.63 (dd, J = 8.1, 1.4 Hz, 1H), 7.76 (d, J = 8.2 Hz, 1H), 8.02 (d, J = 2.3 Hz, |
| | 1H), 8.50 (d, J = 2.7 Hz, 1H) |
| 1184 | 1.08-1.20 (m, 2H), 1.39-1.51 (m, 1H), 1.61-1.69 (m, 2H), 1.76 (td, J = 11.6, 2.5 |
| | Hz, 2H), 2.11 (s, 3H), 2.71 (dt, J = 11.7, 3.4 Hz, 2H), 3.08 (t, J = 6.3 Hz, 2H), 4.16 (d, |
| | J = 5.5 Hz, 2H), 6.42-6.49 (m, 2H), 6.68 (t, J = 5.5 Hz, 1H), 6.81 (d, J = 2.2 Hz, 1H), |
| | 7.04-7.11 (m, 2H), 7.38 (dd, J = 8.6, 2.4 Hz, 1H), 7.60 (d, J = 8.9 Hz, 1H), 7.98- |
| | 8.03 (m, 2H), 8.58 (dd, J = 4.3, 1.8 Hz, 1H) |
| 1185 | 4.19 (2H, d, J = 5.5 Hz), 4.59 (2H, d, J = 5.9 Hz), 6.54 (2H, s), 6.57 (1H, d, J = 8.5, 0.7 |
| | Hz), 6.71 (1H, d, J = 2.3 Hz), 6.89-6.97 (2H, m), 6.99 (1H, d, J = 1.6 Hz), 7.08 (1H, t, |
| | J = 5.9 Hz), 7.45 (1H, dd, J = 8.5, 2.4 Hz), 7.49 (1H, d, J = 1.6 Hz), 7.66 (1H, s), 7.91 |
| | (1H, d, J = 8.8 Hz), 8.04 (1H, t, J = 59.3 Hz), 8.05 (1H, s) |
| 1186 | 1H NMR (MeOD, 500 MHz) δ 1.24-1.36 (2H, m), 1.54-1.68 (1H, m), 1.81 (2H, app |
| | d, J = 13.3 Hz), 2.01 (2H, app t, J = 11.8 Hz), 2.27 (3H, s), 2.89 (2H, app d, J = 11.7 Hz), |
| | 3.00 (6H, s), 3.15 (2H, d, J = 6.9 Hz), 4.32 (2H, s), 6.49 (1H, d, J = 8.6 Hz), 6.83 (1H, d, |
| | J = 9.7 Hz), 7.06 (1H, d, J = 9.7 Hz), 7.47 (1H, dd, J = 8.6, 2.4 Hz), 7.91 (1H, d, J = |
| | 1.9 Hz). Exchangeable protons not evident |
| 1187 | 1H NMR (MeOD, 500 MHz) δ 1.29 (2H, qd, J = 12.6, 3.8 Hz), 1.53-1.66 (1H, m), 1.80 |
| | (2H, app d, J = 13.0 Hz), 1.94-2.02 (2H, m), 2.25 (3H, s), 2.87 (2H, app d, J = 11.6 Hz), |
| | 3.15 (2H, d, J = 6.8 Hz), 4.31 (2H, s), 6.50 (1H, d, J = 8.7 Hz), 6.96 (1H, dd, J = 9.2, |
| | 4.4 Hz), 7.00 (1H, s), 7.50 (1H, dd, J = 8.7, 2.4 Hz), 7.79 (1H, dd, J = 9.2, 1.6 Hz), 7.94 |
| | (1H, d, J = 1.9 Hz), 8.34 (1H, dd, J = 4.4, 1.5 Hz) |
| 1188 | 1.09-1.20 (m, 2H), 1.45-1.49 (m, 1H), 1.62-1.68 (m, 2H), 1.74-1.83 (m, 2H), |
| | 2.12 (s, 3H), 2.70-2.76 (m, 2H), 3.09 (t, J = 6.3 Hz, 2H), 4.20 (d, J = 5.4 Hz, 2H), |
| | 6.45 (d, J = 8.6 Hz, 1H), 6.47-6.53 (m, 1H), 6.78 (d, J = 2.2 Hz, 1H), 7.19 (dd, J = 9.0, |
| | 2.2 Hz, 1H), 7.25-7.31 (m, 1H), 7.38 (dd, J = 8.6, 2.4 Hz, 1H), 7.83 (d, J = 8.9 Hz, |
| | 1H), 8.01 (d, J = 2.4 Hz, 1H), 8.30 (s, 1H), 8.82 (s, 1H). |
| 1189 | 1.14 (2H, qd, J = 12.0, 3.8 Hz), 1.39-1.52 (1H, m), 1.64 (2H, d, J = 12.8 Hz), 1.76 |
| | (2H, td, J = 11.7, 2.5 Hz), 2.11 (3H, s), 2.68-2.75 (2H, m), 3.09 (2H, t, J = 6.3 Hz), |
| | 4.21 (2H, d, J = 5.4 Hz), 6.46 (1H, d, J = 8.6 Hz), 6.52 (1H, t, J = 5.8 Hz), 7.11 (1H, d, |
| | J = 3.0 Hz), 7.36 (1H, t, J = 5.5 Hz), 7.40 (1H, dd, J = 8.6, 2.4 Hz), 8.03 (1H, d, J = 2.4 |
| | Hz), 8.62 (1H, d, J = 1.9 Hz), 8.74 (2H, dd, J = 10.3, 2.5 Hz). |
| 1190 | 1.13 (2H, qd, J = 12.0, 3.9 Hz), 1.40-1.48 (1H, m), 1.60-1.67 (2H, m), 1.76 (2H, td, |
| | J = 11.6, 2.5 Hz), 2.11 (3H, s), 2.71 (2H, d, J = 11.3 Hz), 3.06 (2H, t, J = 6.3 Hz), 4.23 |
| | (2H, d, J = 6.2 Hz), 6.05 (1H, d, J = 7.4 Hz), 6.35 (1H, t, J = 6.2 Hz), 6.38-6.45 (2H, |
| | m), 6.61 (1H, t, J = 7.0 Hz), 7.35-7.42 (2H, m), 7.75 (1H, dd, J = 6.6, 1.0 Hz), 7.79 |
| | (1H, d, J = 1.2 Hz), 7.97 (1H, d, J = 2.4 Hz). |
| 1191 | 1.13 (2H, qd, J = 12.1, 3.9 Hz), 1.36-1.49 (1H, m), 1.63 (2H, d, J = 12.4 Hz), 1.76 (2H, |
| | td, J = 11.7, 2.5 Hz), 2.11 (3H, s), 2.71 (2H, d, J = 11.3 Hz), 3.05 (2H, t, J = 6.4 Hz), 4.14 |
| | (2H, d, J = 6.4 Hz), 5.34-5.45 (1H, m), 6.31-6.42 (2H, m), 6.72 (1H, t, J = 8.2 Hz), 7.13 |
| | (1H, d, J = 8.5 Hz), 7.36 (1H, dd, J = 8.6, 2.4 Hz), 7.89-7.96 (1H, m), 8.00 (1H, s), 11.70 |
| | (1H, s) |
| 1192 | 1.10-1.19 (2H, m), 1.38-1.53 (1H, m), 1.64 (2H, d, J = 12.5 Hz), 1.76 (2H, td, J = 11.7, |
| | 2.5 Hz), 2.11 (3H, s), 2.71 (2H, d, J = 11.4 Hz), 3.08 (2H, t, J = 6.3 Hz), 3.48 (3H, s), |
| | 4.36 (2H, d, J = 5.7 Hz), 6.38-6.48 (2H, m), 6.85-7.00 (3H, m), 7.09-7.15 (1H, m), |
| | 7.16-7.22 (1H, m), 7.41 (1H, dd, J = 8.6, 2.4 Hz), 7.98 (1H, d, J = 2.4 Hz) |
| 1193 | 1.14 (qd, J = 12.0, 3.8 Hz, 2H), 1.39-1.50 (m, 1H), 1.61-1.67 (m, 2H), 1.76 (td, J = |
| | 11.6, 2.5 Hz, 2H), 2.11 (s, 3H), 2.68-2.76 (m, 2H), 3.08 (t, J = 6.3 Hz, 2H), 4.15 (d, |
| | J = 5.5 Hz, 2H), 6.41-6.49 (m, 2H), 6.52 (s, 2H), 6.71 (d, J = 2.3 Hz, 1H), 6.88 (t, J = |
| | 5.5 Hz, 1H), 6.93 (dd, J = 9.1, 2.3 Hz, 1H), 7.37 (dd, J = 8.6, 2.4 Hz, 1H), 7.65 (s, 1H), |
| | 7.90 (d, J = 9.1 Hz, 1H), 7.98 (d, J = 2.4 Hz, 1H) |
| 1194 | 1.14 (qd, J = 12.0, 3.8 Hz, 2H), 1.36-1.50 (m, 1H), 1.59-1.69 (m, 2H), 1.77 (td, J = |
| | 11.6, 2.5 Hz, 2H), 2.11 (s, 3H), 2.72 (d, J = 11.6 Hz, 2H), 3.07 (t, J = 6.3 Hz, 2H), 4.22 |
| | (d, J = 5.3 Hz, 2H), 6.23 (dt, J = 14.8, 5.5 Hz, 1H), 6.38-6.50 (m, 4H), 6.69 (dd, J = |
| | 7.7, 1.2 Hz, 1H), 7.23-7.35 (m, 2H), 7.39 (dd, J = 8.6, 2.4 Hz, 1H), 7.59 (d, J = 5.8 Hz, |
| | 1H), 7.99 (d, J = 2.3 Hz, 1H) |
| 1195 | 1.03-1.21 (m, 2H), 1.39-1.50 (m, 1H), 1.61-1.67 (m, 2H), 1.76 (td, J = 11.7, 2.5 |
| | Hz, 2H), 2.11 (s, 3H), 2.68-2.76 (m, 2H), 3.05-3.11 (m, 2H), 4.15 (d, J = 5.5 Hz, 2H), |
| | 6.24 (s, 2H), 6.41-6.50 (m, 2H), 6.58 (d, J = 2.3 Hz, 1H), 6.80 (t, J = 5.5 Hz, 1H), 6.94 |
| | (dd, J = 9.2, 2.3 Hz, 1H), 7.36 (dd, J = 8.6, 2.4 Hz, 1H), 7.52 (d, J = 2.9 Hz, 1H), 7.87 |
| | (dd, J = 9.1, 2.4 Hz, 1H), 7.97 (d, J = 2.4 Hz, 1H) |
| 1196 | 1.13 (qd, J = 12.0, 3.9 Hz, 2H), 1.38-1.49 (m, 1H), 1.64 (d, J = 12.8 Hz, 2H), 1.76 (td, |
| | J = 11.6, 2.5 Hz, 2H), 2.11 (s, 3H), 2.71 (d, J = 11.3 Hz, 2H), 3.07 (t, J = 6.3 Hz, 2H), |
| | 4.26 (d, J = 5.9 Hz, 2H), 6.30 (s, 1H), 6.35-6.44 (m, 2H), 6.46 (d, J = 6.0 Hz, 1H), |
| | 6.79 (s, 2H), 7.06 (t, J = 6.1 Hz, 1H), 7.34 (dd, J = 8.6, 2.4 Hz, 1H), 7.61 (d, J = 5.9 Hz, |
| | 1H), 7.94 (d, J = 2.4 Hz, 1H), 9.05 (s, 1H) |
| 1197 | 1.13 (qd, J = 12.1, 3.8 Hz, 2H), 1.41-1.49 (m, 1H), 1.61-1.67 (m, 2H), 1.76 (td, J = |
| | 11.6, 2.5 Hz, 2H), 2.11 (s, 3H), 2.68-2.74 (m, 2H), 3.07 (t, J = 6.3 Hz, 2H), 4.23 (d, |
| | J = 5.8 Hz, 2H), 6.12 (d, J = 5.5 Hz, 1H), 6.39-6.46 (m, 2H), 6.55 (d, J = 3.4 Hz, 1H), |
| | 6.89 (t, J = 5.9 Hz, 1H), 7.03 (d, J = 3.5 Hz, 1H), 7.34 (dd, J = 8.6, 2.4 Hz, 1H), 7.76 (d, |
| | J = 5.4 Hz, 1H), 7.96 (d, J = 2.4 Hz, 1H), 11.08 (s, 1H) |
| 1198 | 1.12 (qd, J = 12.0, 3.8 Hz, 2H), 1.42-1.45 (m, 1H), 1.55-1.58 (m, 2H), 1.59-1.78 |
| | (m, 8H), 2.10 (s, 3H), 2.68-2.74 (m, 2H), 2.86-2.90 (m, 4H), 3.07 (t, J = 6.3 Hz, 2H), |
| | 4.20 (d, J = 5.3 Hz, 2H), 6.39-6.48 (m, 2H), 6.64 (d, J = 2.2 Hz, 1H), 6.98 (t, J = 5.6 |
| | Hz, 1H), 7.07 (dd, J = 8.9, 2.2 Hz, 1H), 7.34 (dd, J = 8.6, 2.4 Hz, 1H), 7.70 (d, J = 8.9 |
| | Hz, 1H), 7.85 (s, 1H), 7.98 (d, J = 2.4 Hz, 1H), 8.56 (s, 1H) |
| 1199 | 1.14 (2H, qd, J = 12.1, 3.8 Hz), 1.39-1.51 (1H, m), 1.65 (2H, d, J = 12.1 Hz), 1.72- |
| | 1.81 (2H, m), 2.11 (3H, s), 2.72 (2H, d, J = 11.6 Hz), 3.08 (2H, t, J = 6.3 Hz), 3.90 (2H, |
| | d, J = 6.1 Hz), 5.27 (1H, t, J = 6.1 Hz), 6.26 (1H, d, J = 1.8 Hz), 6.36-6.44 (3H, m), |
| | 6.77 (1H, d, J = 1.8 Hz), 7.11 (1H, d, J = 5.6 Hz), 7.34 (1H, dd, J = 8.5, 2.4 Hz), 7.92 |
| | (1H, d, J = 2.4 Hz), 9.82 (1H, s) |
| 1200 | 1.08-1.20 (2H, m), 1.39-1.52 (1H, m), 1.64 (2H, d, J = 12.9 Hz), 1.76 (2H, t, J = 11.5 |
| | Hz), 2.11 (3H, s), 2.66-2.76 (2H, m), 3.09 (2H, t, J = 6.5 Hz), 3.85 (3H, s), 4.35 (2H, |
| | s), 6.44 (1H, d, J = 8.4 Hz), 6.51 (1H, t, J = 5.9 Hz), 6.84 (1H, d, J = 8.2 Hz), 6.97 (1H, |
| | t, J = 8.1 Hz), 7.25 (1H, d, J = 7.9 Hz), 7.37 (1H, d, J = 8.6 Hz), 7.96 (1H, s), 8.18 (1H, s) |
| 1201 | 1.08-1.20 (2H, m), 1.41-1.52 (1H, m), 1.61-1.70 (2H, m), 1.79 (2H, t, J = 11.4 Hz), |
| | 2.13 (3H, s), 2.73 (2H, d, J = 10.2 Hz), 3.09 (2H, t, J = 6.2 Hz), 4.10 (2H, d, J = 5.1 Hz), |
| | 6.20-6.30 (2H, m), 6.44 (1H, d, J = 8.5 Hz), 6.48 (1H, t, J = 5.7 Hz), 6.67 (1H, dd, J = |
| | 9.0, 2.1 Hz), 6.74 (1H, t, J = 5.5 Hz), 7.36 (1H, dd, J = 8.6, 2.4 Hz), 7.45 (1H, d, J = 9.0 |
| | Hz), 7.97 (1H, d, J = 2.4 Hz), 8.26 (1H, s), 10.76 (1H, s) |
| 1202 | 1.13 (2H, qd, J = 12.0, 3.9 Hz), 1.38-1.50 (1H, m), 1.64 (2H, d, J = 10.8 Hz), 1.76 |
| | (2H, td, J = 11.6, 2.5 Hz), 2.11 (3H, s), 2.29 (3H, s), 2.67-2.75 (2H, m), 3.07 (2H, t, |
| | J = 6.3 Hz), 4.21 (2H, d, J = 5.8 Hz), 6.07 (1H, d, J = 5.5 Hz), 6.20 (1H, d, J = 1.2 Hz), |
| | 6.38-6.45 (2H, m), 6.68 (1H, t, J = 6.0 Hz), 7.33 (1H, dd, J = 8.6, 2.4 Hz), 7.65 (1H, d, |
| | J = 5.4 Hz), 7.94 (1H, d, J = 2.4 Hz), 10.91 (1H, s) |
| 1203 | (500 MHz, MeOD) 1.40-1.51 (2H, m), 1.78-1.90 (1H, m), 1.93-2.01 (2H, m), 2.70- |
| | 2.80 (5H, m), 3.22 (2H, d, J = 6.8 Hz), 3.34-3.38 (2H, m), 4.33 (2H, s), 6.53 (1H, d, |
| | J = 8.7 Hz), 6.77 (1H, d, J = 2.3 Hz), 7.46 (1H, d, J = 2.3 Hz), 7.47-7.53 (2H, m), 8.00 |
| | (1H, d, J = 2.0 Hz), 8.09 (1H, d, J = 6.1 Hz), 9.59 (1H, s) |
| 1204 | 1.16-1.24 (2H, m), 1.46-1.58 (1H, m), 1.69 (2H, d, J = 13.0 Hz), 2.01 (2H, t, J = 11.6 |
| | Hz), 2.24 (3H, s), 2.86 (2H, d, J = 11.4 Hz), 3.09 (2H, t, J = 6.3 Hz), 4.08 (2H, s), 6.37- |
| | 6.46 (2H, m), 6.75 (1H, d, J = 2.3 Hz), 7.24 (1H, d, J = 2.6 Hz), 7.29 (1H, d, J = 2.3 Hz), |
| | 7.37 (1H, dd, J = 8.6, 2.4 Hz), 7.96 (1H, d, J = 2.3 Hz), 10.75 (1H, s) |
| 1205 | 1.12 (2H, qd, J = 12.0, 3.8 Hz), 1.37-1.48 (1H, m), 1.59-1.67 (2H, m), 1.75 (2H, td, |
| | J = 11.6, 2.5 Hz), 2.11 (6H, d, J = 2.8 Hz), 2.71 (2H, d, J = 11.6 Hz), 3.04 (2H, t, J = 6.2 |
| | Hz), 4.60 (2H, d, J = 6.6 Hz), 5.87 (1H, t, J = 6.5 Hz), 6.34-6.43 (3H, m), 6.99 (1H, d, |
| | J = 3.6 Hz), 7.32 (1H, dd, J = 8.6, 2.4 Hz), 7.64 (1H, s), 7.90 (1H, d, J = 2.4 Hz), 10.96 |
| | (1H, s) |
| 1206 | 1.12 (2H, qd, J = 12.0, 3.9 Hz), 1.37-1.48 (1H, m), 1.63 (2H, d, J = 12.7 Hz), 1.75 |
| | (2H, td, J = 11.6, 2.5 Hz), 2.10 (3H, s), 2.66-2.75 (2H, m), 3.04 (2H, t, J = 6.3 Hz), |
| | 4.50 (2H, d, J = 6.7 Hz), 6.35-6.44 (2H, m), 6.56 (1H, dd, J = 3.6, 1.9 Hz), 6.62 (1H, |
| | td, J = 6.7, 2.6 Hz), 7.10-7.15 (1H, m), 7.33 (1H, dd, J = 8.6, 2.4 Hz), 7.82 (1H, d, J = |
| | 5.0 Hz), 7.90 (1H, d, J = 2.4 Hz), 11.23 (1H, s) |
| 1207 | 1.13 (2H, qd, J = 12.0, 3.8 Hz), 1.44 (1H, ddt, J = 11.1, 7.4, 3.6 Hz), 1.64 (2H, d, J = |
| | 12.7 Hz), 1.76 (2H, td, J = 11.6, 2.5 Hz), 2.11 (3H, s), 2.71 (2H, d, J = 10.5 Hz), 3.07 |
| | (2H, t, J = 6.3 Hz), 4.22 (2H, d, J = 5.9 Hz), 6.09 (1H, t, J = 6.0 Hz), 6.38-6.44 (2H, |
| | m), 6.65 (1H, t, J = 2.5 Hz), 7.30 (1H, t, J = 2.7 Hz), 7.33 (1H, s), 7.37 (1H, dd, J = 8.6, |
| | 2.4 Hz), 7.97 (1H, d, J = 2.4 Hz), 8.06 (1H, s), 11.23 (1H, s) |
| 1208 | 1.14 (2H, qd, J = 11.9, 3.7 Hz), 1.40-1.49 (1H, m), 1.61-1.67 (2H, m), 1.76 (2H, td, |
| | J = 11.6, 2.5 Hz), 2.11 (3H, s), 2.68-2.75 (2H, m), 3.09 (2H, t, J = 6.3 Hz), 4.16 (2H, |
| | d, J = 5.4 Hz), 6.45 (1H, d, J = 8.5 Hz), 6.51 (1H, t, J = 5.8 Hz), 6.71 (1H, d, J = 2.0 Hz), |
| | 7.05 (1H, t, J = 5.5 Hz), 7.25 (1H, d, J = 2.1 Hz), 7.37 (1H, dd, J = 8.6, 2.4 Hz), 7.47 |
| | (1H, d, J = 5.8 Hz), 8.00 (1H, d, J = 2.4 Hz), 8.27 (1H, d, J = 5.8 Hz), 9.09 (1H, s) |
| 1209 | 1.12 (2H, qd, J = 12.0, 3.8 Hz), 1.37-1.47 (1H, m), 1.59-1.66 (2H, m), 1.71-1.79 |
| | (2H, m), 2.10 (3H, s), 2.67-2.74 (2H, m), 3.05 (2H, t, J = 6.3 Hz), 4.40 (2H, d, J = 6.3 |
| | Hz), 6.41 (1H, d, J = 8.6 Hz), 6.45 (1H, t, J = 5.8 Hz), 6.83 (1H, t, J = 6.3 Hz), 7.28 (1H, |
| | d, J = 9.0 Hz), 7.36 (1H, dd, J = 8.6, 2.4 Hz), 7.63 (1H, d, J = 6.0 Hz), 7.86 (1H, d, J = |
| | 8.9 Hz), 7.98 (1H, d, J = 2.3 Hz), 8.36 (1H, d, J = 6.0 Hz), 8.97 (1H, s) |
| 1210 | 1.14 (2H, qd, J = 12.1, 3.8 Hz), 1.45 (1H, ddt, J = 11.2, 7.7, 3.8 Hz), 1.64 (2H, d, J = |
| | 12.7 Hz), 1.76 (2H, td, J = 11.6, 2.5 Hz), 2.11 (3H, s), 2.72 (2H, d, J = 11.3 Hz), 2.82 |
| | (3H, d, J = 4.6 Hz), 3.08 (2H, t, J = 6.3 Hz), 4.18 (2H, d, J = 5.5 Hz), 6.45 (1H, d, J = 8.8 |
| | Hz), 6.48 (1H, t, J = 5.8 Hz), 6.74 (1H, d, J = 2.2 Hz), 6.97 (1H, t, J = 5.6 Hz), 7.15 (1H, |
| | d, J = 2.2 Hz), 7.37 (1H, dd, J = 8.6, 2.4 Hz), 7.43 (1H, d, J = 5.4 Hz), 8.00 (1H, d, J = |
| | 2.4 Hz), 8.19 (1H, d, J = 5.8 Hz), 8.51 (1H, q, J = 4.5 Hz), 9.04 (1H, s) |
| 1211 | 1.07-1.19 (2H, m), 1.38-1.51 (1H, m), 1.64 (2H, d, J = 12.7 Hz), 1.71-1.81 (2H, |
| | m), 2.11 (3H, s), 2.71 (2H, d, J = 10.6 Hz), 3.08 (2H, t, J = 6.3 Hz), 4.16 (2H, d, J = 5.5 |
| | Hz), 4.86-4.94 (2H, m), 5.29-5.39 (1H, m), 6.39-6.50 (2H, m), 6.55 (1H, s), 6.89 |
| | (1H, t, J = 5.7 Hz), 7.15 (1H, s), 7.33-7.40 (2H, m), 7.99 (1H, d, J = 2.4 Hz), 8.17 (1H, |
| | d, J = 5.7 Hz), 9.02 (1H, s) |
| 1212 | 1.10-1.19 (2H, m), 1.40-1.50 (1H, m), 1.65 (2H, d, J = 12.8 Hz), 1.73-1.80 (2H, |
| | m), 2.11 (3H, s), 2.72 (2H, d, J = 11.2 Hz), 3.08 (2H, t, J = 6.3 Hz), 4.08-4.12 (2H, m), |
| | 6.10 (1H, t, J = 5.8 Hz), 6.41-6.46 (2H, m), 7.03-7.09 (1H, m), 7.37 (1H, dd, J = 8.6, |
| | 2.4 Hz), 7.94 (1H, d, J = 2.5 Hz), 7.97 (1H, d, J = 2.4 Hz), 8.14 (1H, s), 12.33 (1H, s) |
| 1213 | 1.13 (2H, qd, J = 12.1, 3.8 Hz), 1.39-1.49 (1H, m), 1.60-1.68 (2H, m), 1.76 (2H, td), |
| | 2.11 (3H, s), 2.71 (2H, dt, J = 11.7, 3.3 Hz), 3.07 (2H, t, J = 6.3 Hz), 4.22 (2H, d, J = |
| | 5.7 Hz), 6.09 (1H, t, J = 6.0 Hz), 6.38-6.44 (2H, m), 6.65 (1H, d, J = 3.0 Hz), 7.30 (1H, |
| | d, J = 3.1 Hz), 7.33 (1H, s), 7.37 (1H, dd, J = 8.6, 2.4 Hz), 7.97 (1H, d, J = 2.4 Hz), 8.06 |
| | (1H, s), 11.23 (1H, s) |
| 1214 | 1.13 (2H, qd, J = 12.0, 3.8 Hz), 1.38-1.50 (1H, m), 1.60-1.68 (2H, m), 1.76 (2H, td, |
| | J = 11.7, 2.5 Hz), 2.11 (3H, s), 2.71 (2H, dt, J = 11.7, 3.4 Hz), 3.07 (2H, t, J = 6.2 Hz), |
| | 4.24 (2H, d, J = 5.3 Hz), 6.39-6.46 (2H, m), 6.58 (1H, dd, J = 8.5, 4.2 Hz), 6.78 (1H, t, |
| | J = 5.7 Hz), 7.20 (1H, dd, J = 10.5, 8.5 Hz), 7.37 (1H, dd, J = 8.6, 2.4 Hz), 7.99 (1H, d, |
| | J = 2.3 Hz), 8.13 (1H, d, J = 6.1 Hz), 8.53 (1H, d, J = 6.0 Hz), 9.29 (1H, s) |
| 1215 | 1.14 (2H, qd, J = 12.0, 3.9 Hz), 1.40-1.51 (1H, m), 1.61-1.69 (2H, m), 1.77 (2H, td, |
| | J = 11.6, 2.5 Hz), 2.11 (3H, s), 2.23 (3H, s), 2.72 (2H, d, J = 11.2 Hz), 3.07 (2H, t, J = |
| | 6.3 Hz), 4.16 (2H, d, J = 5.8 Hz), 5.81 (1H, t, J = 6.0 Hz), 5.89 (1H, s), 6.38 (1H, t, J = |
| | 5.8 Hz), 6.40-6.44 (2H, m), 6.48-6.53 (1H, m), 6.97 (1H, dd, J = 3.1, 2.3 Hz), 7.36 |
| | (1H, dd, J = 8.6, 2.4 Hz), 7.96 (1H, d, J = 2.3 Hz), 10.59 (1H, s) |
| 1216 | 1.07-1.19 (2H, m), 1.38-1.49 (1H, m), 1.64 (2H, d, J = 12.8 Hz), 1.72-1.81 (2H, |
| | m), 2.11 (3H, s), 2.34 (3H, s), 2.72 (2H, d, J = 10.8 Hz), 3.07 (2H, t, J = 6.2 Hz), 3.59 |
| | (3H, s), 4.22 (2H, d, J = 5.8 Hz), 6.12 (1H, d, J = 5.5 Hz), 6.30 (1H, s), 6.37-6.46 (2H, |
| | m), 6.76 (1H, t, J = 6.1 Hz), 7.32 (1H, dd, J = 8.6, 2.4 Hz), 7.72 (1H, d, J = 5.4 Hz), |
| | 7.94 (1H, d, J = 2.4 Hz) |
| 1217 | 1.08-1.20 (2H, m), 1.40-1.52 (1H, m), 1.65 (2H, d, J = 12.7 Hz), 1.81 (2H, t, J = 11.5 |
| | Hz), 2.14 (3H, s), 2.74 (2H, d, J = 11.4 Hz), 3.07 (2H, t, J = 6.3 Hz), 3.86 (3H, s), 4.29 |
| | (2H, d, J = 5.7 Hz), 6.34 (1H, d, J = 5.4 Hz), 6.42 (1H, d, J = 8.6 Hz), 6.46 (1H, t, J = 5.8 |
| | Hz), 7.05 (1H, dd, J = 9.2, 2.7 Hz), 7.14 (1H, d, J = 2.7 Hz), 7.36 (1H, dd, J = 8.6, 2.4 |
| | Hz), 7.59 (1H, t, J = 5.8 Hz), 7.99 (1H, d, J = 2.4 Hz), 8.14 (1H, d, J = 9.3 Hz), 8.25 (1H, |
| | d, J = 5.4 Hz) |
| 1218 | 1.07-1.20 (2H, m), 1.41-1.50 (1H, m), 1.64 (2H, d, J = 11.4 Hz), 1.72-1.81 (2H, |
| | m), 2.12 (3H, s), 2.72 (2H, d, J = 11.4 Hz), 3.08 (2H, t, J = 6.3 Hz), 4.27 (2H, d, J = 5.6 |
| | Hz), 6.16 (1H, d, J = 5.5 Hz), 6.44 (1H, d, J = 8.6 Hz), 6.49 (1H, t, J = 5.8 Hz), 7.36 (1H, |
| | dd, J = 8.6, 2.4 Hz), 7.58 (1H, t, J = 5.7 Hz), 7.95 (1H, d, J = 5.5 Hz), 7.98 (1H, d, J = |
| | 2.4 Hz), 8.12 (1H, s), 12.99 (1H, s) |
| 1219 | 1.09-1.19 (2H, m), 1.39-1.50 (1H, m), 1.64 (2H, d, J = 10.9 Hz), 1.76 (2H, td, J = |
| | 11.6, 2.5 Hz), 2.11 (3H, s), 2.65 (3H, d, J = 4.9 Hz), 2.72 (2H, d, J = 11.4 Hz), 3.08 (2H, |
| | t, J = 6.3 Hz), 3.99 (2H, d, J = 5.7 Hz), 5.50 (1H, d, J = 2.0 Hz), 5.78 (1H, q, J = 4.9 Hz), |
| | 5.85 (1H, dd, J = 5.8, 2.0 Hz), 6.36 (1H, t, J = 5.7 Hz), 6.40-6.47 (2H, m), 7.29 (1H, |
| | dd, J = 8.6, 2.4 Hz), 7.49 (1H, d, J = 5.8 Hz), 7.90 (1H, d, J = 2.4 Hz) |
| 1220 | 1.08-1.20 (2H, m), 1.40-1.51 (1H, m), 1.65 (2H, d, J = 12.2 Hz), 1.80 (2H, t, J = 11.6 |
| | Hz), 2.13 (3H, s), 2.74 (2H, d, J = 11.1 Hz), 2.81 (3H, d, J = 4.5 Hz), 3.07 (2H, t, J = 6.3 |
| | Hz), 4.11 (2H, d, J = 5.9 Hz), 5.63 (1H, t, J = 6.1 Hz), 6.37-6.45 (2H, m), 6.75 (1H, d, |
| | J = 2.0 Hz), 7.10 (1H, d, J = 2.1 Hz), 7.23 (1H, d, J = 2.8 Hz), 7.37 (1H, dd, J = 8.6, 2.4 |
| | Hz), 7.97 (1H, d, J = 2.3 Hz), 8.38-8.45 (1H, m), 10.80 (1H, s) |
| 1221 | 1.14 (2H, qd, J = 12.0, 3.8 Hz), 1.38-1.50 (1H, m), 1.60-1.68 (2H, m), 1.77 (2H, td, |
| | J = 11.6, 2.5 Hz), 2.11 (3H, s), 2.68-2.75 (2H, m), 3.04-3.11 (2H, m), 4.30 (2H, d, |
| | J = 5.6 Hz), 5.88 (1H, t, J = 5.6 Hz), 6.39 (1H, d, J = 5.6 Hz), 6.45 (1H, d, J = 8.6 Hz), |
| | 6.50 (1H, t, J = 5.8 Hz), 7.40 (1H, dd, J = 8.6, 2.4 Hz), 7.95 (1H, d, J = 5.5 Hz), 8.00 |
| | (1H, d, J = 2.4 Hz), 8.10 (1H, s), 12.21 (1H, s) |
| 1222 | 1.14 (2H, qd, J = 12.0, 3.9 Hz), 1.40-1.51 (1H, m), 1.60-1.68 (2H, m), 1.76 (2H, td, |
| | J = 11.6, 2.5 Hz), 2.11 (3H, s), 2.66-2.76 (2H, m), 3.09 (2H, t, J = 6.3 Hz), 4.06 (2H, |
| | d, J = 5.4 Hz), 6.22 (1H, d, J = 2.2 Hz), 6.38-6.51 (4H, m), 7.16 (1H, d, J = 1.3 Hz), |
| | 7.35 (1H, dd, J = 8.6, 2.4 Hz), 7.48 (1H, s), 7.97 (1H, d, J = 2.3 Hz), 8.11 (1H, d, J = 7.3 Hz) |
| 1223 | 1.13 (2H, qd, J = 12.1, 3.8 Hz), 1.38-1.51 (1H, m), 1.59-1.67 (2H, m), 1.71-1.79 |
| | (2H, m), 2.11 (3H, s), 2.71 (2H, d, J = 10.7 Hz), 3.07 (2H, t, J = 6.3 Hz), 4.44 (2H, d, J = |
| | 5.9 Hz), 6.35 (1H, t, J = 5.8 Hz), 6.39 (1H, d, J = 8.5 Hz), 6.56-6.63 (2H, m), 6.84 (1H, |
| | t, J = 5.9 Hz), 7.08 (1H, t, J = 2.6 Hz), 7.36 (1H, dd, J = 8.5, 2.4 Hz), 7.57 (1H, d, J = |
| | 5.9 Hz), 7.93 (1H, d, J = 2.4 Hz), 11.08 (1H, s) |
| 1224 | (500 MHz, Methanol-d4) δ 1.22-1.34 (2H, m), 1.53-1.66 (1H, m), 1.75-1.83 (2H, |
| | m), 1.95-2.04 (2H, m), 2.25 (3H, s), 2.81-2.91 (2H, m), 3.14 (2H, d, J = 6.9 Hz), |
| | 4.42 (2H, s), 6.50 (1H, dd, J = 8.7, 0.8 Hz), 7.29 (1H, dd, J = 8.9, 7.9 Hz), 7.47 (1H, dd, |
| | J = 8.7, 2.4 Hz), 7.66 (1H, dt, J = 6.1, 1.0 Hz), 7.71 (1H, dd, J = 8.9, 1.2 Hz), 7.95 (1H, |
| | dd, J = 2.5, 0.9 Hz), 8.22 (1H, d, J = 6.1 Hz), 8.91 (1H , dd, J = 1.9, 1.0 Hz) |
| 1225 | (500 MHz, Methanol-d4) 1.29 (2H, qd, J = 12.4, 3.8 Hz), 1.55-1.65 (1H, m), 1.76- |
| | 1.84 (2H, m), 1.95-2.03 (2H, m), 2.25 (3H, s), 2.82-2.92 (2H, m), 3.15 (2H, d, J = |
| | 6.9 Hz), 3.75 (3H, s), 4.37 (2H, s), 6.23 (1H, d, J = 5.7 Hz), 6.50 (1H, dd, J = 8.7, 0.8 |
| | Hz), 6.53 (1H, d, J = 3.5 Hz), 7.00 (1H, d, J = 3.5 Hz), 7.46 (1H, dd, J = 8.7, 2.4 Hz), |
| | 7.83 (1H, d, J = 5.7 Hz, 1H), 7.93 (1H, dd, J = 2.4, 0.9 Hz) |
| 1226 | (500 MHz, Methanol-d4) 1.29 (2H, qd, J = 12.2, 3.8 Hz), 1.50-1.68 (1H, m), 1.76- |
| | 1.84 (2H, m), 1.99 (2H, td, J = 12.1, 2.6 Hz), 2.25 (3H, s), 2.48 (3H, d, J = 1.1 Hz), |
| | 2.87 (2H, d, J = 11.3 Hz), 3.15 (2H, d, J = 6.9 Hz), 4.36 (2H, s), 6.16 (1H, d, J = 5.7 Hz), |
| | 6.52 (1H, d, J = 8.7 Hz), 6.78 (1H, d, J = 1.3 Hz), 7.48 (1H, dd, J = 8.7, 2.4 Hz), 7.72 |
| | (1H, d, J = 5.7 Hz), 7.95 (1H, d, J = 2.3 Hz) |
| 1227 | (500 MHz, Methanol-d4) 1.20-1.36 (2H, m), 1.52-1.68 (1H, m), 1.74-1.86 (2H, |
| | m), 1.98 (2H, td, J = 12.0, 2.6 Hz), 2.24 (3H, s), 2.80-2.93 (2H, m), 3.15 (2H, d, J = |
| | 6.9 Hz), 4.59 (2H, s), 6.50 (1H, d, J = 8.7 Hz), 6.55 (1H, d, J = 3.5 Hz), 7.05 (1H, d, J = |
| | 3.5 Hz), 7.48 (1H, dd, J = 8.7, 2.4 Hz), 7.94 (1H, d, J = 2.3 Hz), 8.12 (1H, s) |
| 1228 | 1.33 (2H, brs), 1.76 (1H, brs), 1.85 (2H, d, J = 14.1 Hz), 2.32 (3H, s), 2.69 (3H, s), |
| | 2.84 (2H, brs), 3.05-3.17 (2H, m), 3.36 (2H, brs, obscured by H20), 4.17 (2H, d, J = |
| | 5.9 Hz), 5.77 (1H, t, J = 6.2 Hz), 6.00 (1H, d, J = 7.6 Hz), 6.23 (1H, s), 6.41 (1H, d, J = |
| | 8.5 Hz), 6.47-6.56 (2H, m), 6.69 (1H, t, J = 7.8 Hz), 7.38 (1H, dd, J = 8.5, 2.4 Hz), |
| | 7.95 (1H, d, J = 2.3 Hz), 9.10 (1H, brs), 10.58 (1H, d, J = 2.2 Hz) |
| 1229 | 1.13 (2H, qd, J = 12.0, 3.9 Hz), 1.38-1.50 (1H, m), 1.58-1.69 (2H, m), 1.70-1.81 |
| | (2H, m), 2.11 (3H, s), 2.65-2.76 (2H, m), 3.06 (2H, t, J = 6.3 Hz), 4.36 (2H, d, J = 6.1 |
| | Hz), 6.26 (1H, d, J = 5.5 Hz), 6.35-6.47 (2H, m), 7.02 (1H, s), 7.37 (1H, dd, J = 8.6, |
| | 2.4 Hz), 7.81 (1H, d, J = 5.5 Hz), 7.92-8.03 (2H, m). One exchangeable proton not |
| | observed. |
| 1230 | 1.05-1.20 (2H, m), 1.37-1.51 (1H, m), 1.65 (2H, d, J = 12.7 Hz), 1.76 (2H, td, J = |
| | 11.6, 2.6 Hz), 2.11 (3H, s), 2.72 (2H, d, J = 9.8 Hz), 3.07 (2H, t, J = 6.3 Hz), 4.18 (2H, |
| | d, J = 5.8 Hz), 5.93 (1H, t, J = 5.9 Hz), 6.03 (1H, d, J = 7.6 Hz), 6.33-6.44 (2H, m), |
| | 6.55-6.61 (1H, m), 6.62 (1H, d, J = 8.1 Hz), 6.79 (1H, t, J = 7.8 Hz), 7.02-7.10 (1H, |
| | m), 7.37 (1H, d, J = 8.6 Hz), 7.92-7.99 (1H, m), 10.76 (1H, s) |
| 1231 | 1.08-1.18 (2H, m), 1.26 (6H, d, J = 6.9 Hz), 1.39-1.51 (1H, m), 1.64 (2H, d, J = 11.9 |
| | Hz), 1.76 (2H, td, J = 2.5, 11.5 Hz), 2.11 (3H, s), 2.71 (2H, d, J = 11.6 Hz), 2.94 (1H, |
| | hept, J = 6.5 Hz), 3.07 (2H, t, J = 6.2 Hz), 4.20 (2H, d, J = 5.5 Hz), 6.08 (1H, d, J = 5.5 |
| | Hz), 6.23 (1H, s), 6.36-6.46 (2H, m), 6.71 (1H, t, J = 6.0 Hz), 7.33 (1H, dd, J = 2.4, |
| | 8.6 Hz), 7.68 (1H, d, J = 5.5 Hz), 7.95 (1H, d, J = 2.3 Hz), 10.97 (1H, s) |
| 1232 | 1.13 (2H, qd, J = 3.8, 12.0 Hz), 1.38-1.50 (1H, m), 1.64 (2H, d, J = 11.1 Hz), 1.76 |
| | (2H, td, J = 2.5, 11.7 Hz), 2.11 (3H, s), 2.36 (3H, s), 2.71 (2H, d, J = 11.6 Hz), 3.06 |
| | (2H, t, J = 6.2 Hz), 4.20 (2H, d, J = 6.0 Hz), 5.90 (1H, t, J = 6.1 Hz), 6.32 (1H, s), 6.36- |
| | 6.43 (2H, m), 7.30 (1H, s), 7.36 (1H, dd, J = 2.4, 8.6 Hz), 7.94 (1H, s), 7.96 (1H, d, J = |
| | 2.3 Hz), 10.95 (1H, s) |
| 1233 | 1.14 (2H, qd, J = 12.0, 3.8 Hz), 1.39-1.50 (1H, m), 1.61-1.68 (2H, m), 1.76 (2H, td, |
| | J = 11.5, 2.5 Hz), 2.11 (3H, s), 2.71 (2H, d, J = 11.6 Hz), 3.08 (2H, t, J = 6.3 Hz), 3.73 |
| | (3H, s), 4.18 (2H, d, J = 5.8 Hz), 5.51 (1H, s), 6.39-6.45 (2H, m), 6.47 (1H, d, J = 3.4 |
| | Hz), 6.83 (1H, d, J = 3.4 Hz), 6.91 (1H, t, J = 5.9 Hz), 7.33 (1H, dd, J = 8.5, 2.4 Hz), |
| | 7.94 (1H, d, J = 2.4 Hz), 10.79 (1H, s) |
| 1234 | 1.14 (2H, qd, J = 12.0, 3.8 Hz), 1.40-1.50 (1H, m), 1.61-1.68 (2H, m), 1.72-1.80 |
| | (2H, m), 2.11 (3H, s), 2.14-2.16 (3H, m), 2.68-2.74 (2H, m), 3.07 (2H, t, J = 6.3 Hz), |
| | 4.06 (2H, d, J = 6.0 Hz), 5.51 (1H, t, J = 6.1 Hz), 6.37-6.44 (2H, m), 6.98-7.02 (2H, |
| | m), 7.38 (1H, dd, J = 8.5, 2.4 Hz), 7.75 (1H, d, J = 2.5 Hz), 7.96 (1H, d, J = 2.3 Hz), |
| | 10.71 (1H, s) |
| 1235 | 1.08-1.19 (2H, m), 1.39-1.50 (1H, m), 1.61-1.67 (2H, m), 1.76 (2H, td, J = 11.6, |
| | 2.5 Hz), 2.11 (3H, s), 2.30 (3H, d, J = 0.9 Hz), 2.67-2.78 (2H, m), 3.07 (2H, t, J = 6.3 |
| | Hz), 4.02 (2H, d, J = 6.0 Hz), 5.46 (1H, t, J = 6.1 Hz), 5.86 (1H, d, J = 1.1 Hz), 6.34- |
| | 6.46 (2H, m), 6.93 (1H, d, J = 2.6 Hz), 7.36 (1H, dd, J = 8.6, 2.4 Hz), 7.62 (1H, d, J = |
| | 2.5 Hz), 7.94 (1H, d, J = 2.3 Hz), 10.88 (1H, s) |
| 1236 | 1.14 (2H, qd, J = 12.0, 3.9 Hz), 1.40-1.49 (1H, m), 1.60-1.67 (2H, m), 1.72-1.79 |
| | (2H, m), 2.11 (3H, s), 2.69-2.75 (2H, m), 3.07 (2H, t, J = 6.3 Hz), 3.28 (2H, s), 4.15 |
| | (2H, d, J = 5.9 Hz), 6.25 (1H, d, J = 6.0 Hz), 6.42 (1H, d, J = 8.5 Hz), 6.46 (1H, t, J = 5.8 |
| | Hz), 6.56 (1H, t, J = 5.9 Hz), 7.31 (1H, dd, J = 8.6, 2.4 Hz), 7.64 (1H, d, J = 6.0 Hz), |
| | 7.92 (1H, d, J = 2.4 Hz) |
| 1237 | 1.14 (2H, qd, J = 11.9, 3.9 Hz), 1.40-1.51 (1H, m), 1.60-1.69 (2H, m), 1.81 (2H, t, |
| | J = 11.3 Hz), 2.13 (3H, s), 2.74 (2H, d, J = 11.0 Hz), 3.07 (2H, t, J = 6.3 Hz), 4.22 (2H, d, |
| | J = 5.8 Hz), 6.22 (1H, d, J = 1.4 Hz), 6.38-6.46 (3H, m), 6.73 (1H, t, J = 2.6 Hz), 6.96 |
| | (1H, s), 7.30 (1H, t, J = 2.8 Hz), 7.37 (1H, dd, J = 8.6, 2.4 Hz), 7.97 (1H, d, J = 2.4 Hz), |
| | 11.21 (1H, s) |
| 1238 | 1.13 (2H, qd, J = 12.1, 3.9 Hz), 1.39-1.49 (1H, m), 1.60-1.68 (2H, m), 1.76 (2H, td, |
| | J = 11.6, 2.5 Hz), 2.11 (3H, s), 2.71 (2H, d, J = 11.5 Hz), 3.07 (2H, t, J = 6.3 Hz), 3.76 |
| | (3H, s), 4.22 (2H, d, J = 5.8 Hz), 6.26 (1H, s), 6.38-6.44 (2H, m), 6.47 (1H, t, J = 5.8 |
| | Hz), 6.72 (1H, d, J = 3.1 Hz), 7.02 (1H, s), 7.27 (1H, d, J = 3.1 Hz), 7.36 (1H, dd, J = |
| | 8.5, 2.4 Hz), 7.96 (1H, d, J = 2.3 Hz) |
| 1239 | (500 MHz, Methanol-d4) 1.23-1.37 (5H, m), 1.54-1.67 (1H, m), 1.76-1.85 (2H, |
| | m), 1.99 (2H, t, J = 11.8 Hz), 2.25 (3H, s), 2.74 (2H, q, J = 7.6 Hz), 2.83-2.93 (2H, m), |
| | 3.14 (2H, d, J = 6.9 Hz), 4.36 (2H, s), 6.17 (1H, d, J = 5.6 Hz), 6.23 (1H, s), 6.50 (1H, d, |
| | J = 8.7 Hz), 7.46 (1H, dd, J = 8.7, 2.3 Hz), 7.69 (1H, dd, J = 5.6, 1.6 Hz), 7.92 (1H, d, |
| | J = 2.4 Hz) |
| 1240 | (500 MHz, Methanol-d4) 1.22-1.35 (2H, m), 1.52-1.67 (1H, m), 1.74-1.84 (2H, |
| | m), 1.91-2.01 (2H, m), 2.24 (3H, s), 2.36 (3H, s), 2.81-2.92 (2H, m), 3.13 (3H, d, J = |
| | 6.9 Hz), 4.26 (2H, s), 6.16 (1H, d, J = 7.6 Hz), 6.47 (1H, d, J = 8.6 Hz), 6.53 (1H, d, J = |
| | 3.2 Hz), 6.68 (1H, d, J = 7.6 Hz), 7.09 (1H, d, J = 3.2 Hz), 7.48 (1H, dd, J = 8.7, 2.3 Hz), |
| | 7.92 (1H, d, J = 2.3 Hz) |
| 1241 | 1.14 (2H, qd, J = 11.9, 3.8 Hz), 1.35-1.53 (1H, m), 1.65 (2H, d, J = 12.8 Hz), 1.73- |
| | 1.81 (2H, m), 2.11 (3H, s), 2.72 (2H, d, J = 11.4 Hz), 3.07 (2H, t, J = 6.3 Hz), 4.17 (2H, |
| | d, J = 5.9 Hz), 5.99 (1H, d, J = 1.6 Hz), 6.35 (1H, t, J = 5.9 Hz), 6.42 (2H, d, J = 8.1 Hz), |
| | 6.62 (2H, t, J = 2.2 Hz), 7.09 (1H, d, J = 3.1 Hz), 7.35 (1H, dd, J = 8.6, 2.4 Hz), 7.95 |
| | (1H, d, J = 2.3 Hz), 10.90 (1H, s) |
| 1242 | (500 MHz, Methanol-d4) 1.28 (2H, qd, J = 12.3, 3.9 Hz), 1.51-1.67 (1H, m), 1.74- |
| | 1.83 (2H, m), 1.98 (2H, app td, J = 11.9, 2.6 Hz), 2.24 (3H, s), 2.82-2.91 (2H, m), |
| | 3.13 (2H, d, J = 6.9 Hz), 3.86 (3H, s), 4.22 (2H, s), 6.16 (1H, d, J = 8.0 Hz), 6.44 (1H, d, |
| | J = 8.1 Hz), 6.47 (1H, d, J = 8.6 Hz), 6.51 (1H, d, J = 3.1 Hz), 7.08 (1H, d, J = 3.2 Hz), |
| | 7.47 (1H, dd, J = 8.7, 2.4 Hz), 7.91 (1H, d, J = 2.3 Hz) |
| 1243 | (500 MHz, Methanol-d4) 1.29 (2H, qd, J = 12.5, 3.8 Hz), 1.51-1.67 (1H, m), 1.73- |
| | 1.84 (2H, m), 1.99 (2H, app td, J = 12.1, 2.6 Hz), 2.25 (3H, s), 2.81-2.94 (2H, m), |
| | 3.15 (2H, d, J = 6.9 Hz), 4.35 (2H, s), 6.23 (1H, d, J = 5.9 Hz), 6.45 (1H, s), 6.51 (1H, d, |
| | J = 8.7 Hz), 7.45 (1H, dd, J = 8.7, 2.4 Hz), 7.74 (1H, d, J = 5.9 Hz), 7.92 (1H, d, J = 2.3 Hz) |
| 1244 | 1.13 (2H, qd, J = 12.1, 3.9 Hz), 1.39-1.50 (1H, m), 1.59-1.68 (2H, m), 1.76 (2H, td, |
| | J = 11.6, 2.5 Hz), 2.11 (3H, s), 2.46 (3H, s), 2.71 (2H, d, J = 11.5 Hz), 3.07 (2H, t, J = |
| | 6.3 Hz), 4.18 (2H, d, J = 5.9 Hz), 5.84 (1H, t, J = 6.1 Hz), 6.34-6.45 (2H, m), 6.64 (1H, |
| | d, J = 3.1 Hz), 7.20 (1H, s), 7.29 (1H, d, J = 3.1 Hz), 7.36 (1H, dd, J = 8.6, 2.4 Hz), 7.96 |
| | (1H, d, J = 2.3 Hz), 11.17 (1H, s) |
| 1245 | 1.14 (2H, qd, J = 12.0, 3.8 Hz), 1.39-1.52 (1H, m), 1.64 (2H, d, J = 11.5 Hz), 1.76 |
| | (2H, td, J = 11.6, 2.5 Hz), 2.11 (3H, s), 2.72 (2H, d, J = 11.5 Hz), 3.08 (2H, t, J = 6.3 |
| | Hz), 4.10 (2H, d, J = 5.4 Hz), 6.16 (2H, s), 6.40 (1H, d, J = 2.1 Hz), 6.44 (1H, d, J = 8.6 |
| | Hz), 6.48 (1H, t, J = 5.8 Hz), 6.56-6.65 (2H, m), 6.72 (1H, t, J = 5.5 Hz), 7.35 (1H, dd, |
| | J = 8.6, 2.4 Hz), 7.56 (1H, d, J = 5.9 Hz), 7.97 (1H, d, J = 2.4 Hz) |
| | 1.14 (2H, qd, J = 11.9, 3.8 Hz), 1.39-1.50 (1H, m), 1.61-1.68 (2H, m), 1.76 (2H, td, |
| | J = 11.6, 2.5 Hz), 2.11 (3H, s), 2.30 (3H, s), 2.68-2.75 (2H, m), 3.07 (2H, t, J = 6.3 |
| 1246 | Hz), 4.21 (2H, d, J = 5.9 Hz), 6.02 (1H, s), 6.39-6.46 (2H, m), 6.48 (1H, dd, J = 3.4, |
| | 1.8 Hz), 6.75 (1H, t, J = 6.0 Hz), 6.93 (1H, dd, J = 3.5, 2.1 Hz), 7.34 (1H, dd, J = 8.6, |
| | 2.4 Hz), 7.96 (1H, d, J = 2.4 Hz), 10.89 (1H, s) |
| 1247 | 1.14 (2H, qd, J = 12.0, 3.8 Hz), 1.41-1.49 (1H, m), 1.60-1.67 (2H, m), 1.72-1.80 |
| | (2H, m), 2.07-2.14 (4H, m), 2.71 (3H, d, J = 11.3 Hz), 3.08 (2H, t, J = 6.2 Hz), 3.82 |
| | (3H, s), 4.24 (2H, d, J = 5.4 Hz), 6.17 (1H, d, J = 5.6 Hz), 6.43 (1H, d, J = 8.6 Hz), 6.47 |
| | (1H, t, J = 5.8 Hz), 7.31-7.40 (2H, m), 7.43 (1H, s), 7.92 (1H, d, J = 5.5 Hz), 7.97 (1H, |
| | d, J = 2.4 Hz) |
| 1248 | 1.15 (2H, qd, J = 12.0, 3.9 Hz), 1.41-1.50 (1H, m), 1.61-1.69 (2H, m), 1.73-1.82 |
| | (2H, m), 2.12 (3H, s), 2.69-2.75 (2H, m), 3.09 (2H, t, J = 6.2 Hz), 3.76 (3H, s), 4.24 |
| | (2H, d, J = 5.2 Hz), 6.31 (1H, d, J = 5.7 Hz), 6.46 (1H, d, J = 8.6 Hz), 6.52 (1H, t, J = 5.7 |
| | Hz), 7.37 (1H, dd, J = 8.6, 2.4 Hz), 7.88 (1H, d, J = 5.6 Hz), 7.91 (1H, s), 7.99 (1H, d, |
| | J = 2.4 Hz), 8.35 (1H, t, J = 5.3 Hz) |
| 1249 | 1.24 (2H, qd, J = 13.3, 12.9, 3.9 Hz), 1.59-1.70 (3H, m), 1.81 (2H, td, J = 11.7, 2.3 |
| | Hz), 2.13 (3H, s), 2.30 (3H, s), 2.70-2.77 (2H, m), 4.05 (2H, d, J = 6.1 Hz), 4.36 (2H, |
| | d, J = 6.1 Hz), 6.06 (1H, d, J = 5.5 Hz), 6.21 (1H, s), 6.74 (1H, d, J = 8.5 Hz), 6.84 (1H, |
| | t, J = 6.2 Hz), 7.59-7.71 (2H, m), 8.13 (1H, d, J = 2.4 Hz), 10.95 (1H, s) |
| 1250 | 3.76 (2H, t, J = 5.5 Hz), 3.90-4.03 (5H, m), 4.33 (2H, d, J = 5.8 Hz), 4.71 (2H, s), 6.48 |
| | (2H, s), 6.54 (1H, d, J = 7.7 Hz), 6.60 (1H, t, J = 6.0 Hz), 6.99 (1H, d, J = 8.7 Hz), 7.10- |
| | 7.19 (2H, m), 7.30 (1H, d, J = 8.3 Hz), 7.61 (1H, dd, J = 8.7, 2.4 Hz), 7.73 (1H, d, J = |
| | 6.1 Hz), 8.20 (1H, d, J = 2.4 Hz) |
| 1251 | (Methanol-d4, 500 MHz) 1.25-1.39 (m, 2H), 1.56-1.68 (m, 1H), 1.79-1.86 (m, |
| | 2H), 1.98-2.06 (m, 2H), 2.28 (s, 3H), 2.85-2.96 (m, 2H), 3.18 (d, J = 6.9 Hz, 2H), |
| | 4.35 (s, 2H), 6.54 (d, J = 8.6 Hz, 1H), 6.61 (s, 1H), 7.29(s, 1H), 7.51 (dd, J = 8.7, 2.4 |
| | Hz, 1H), 7.95-8.00 (m, 2H) |
| 1252 | 1.13 (4H, qd, J = 12.0, 3.8 Hz), 1.41-1.49 (2H, m), 1.64 (4H, d, J = 12.6 Hz), 1.72- |
| | 1.80 (4H, m), 2.11 (5H, s), 2.71 (5H, d, J = 11.2 Hz), 3.07 (4H, t, J = 6.2 Hz), 4.25 (4H, |
| | d, J = 5.7 Hz), 5.75 (1H, s), 6.15 (2H, d, J = 5.6 Hz), 6.42 (2H, d, J = 8.6 Hz), 6.45 (2H, |
| | t, J = 5.7 Hz), 7.14-7.21 (4H, m), 7.27 (2H, s), 7.35 (2H, dd, J = 8.6, 2.4 Hz), 7.55 |
| | (2H, s), 7.85 (2H, d, J = 5.5 Hz), 7.97 (2H, d, J = 2.4 Hz) |
| 1253 | 1.09-1.21 (2H, qd, J = 12.0, 3.9 Hz), 1.42-1.50 (1H, m), 1.62-1.68 (2H, m), 1.77- |
| | 1.85 (2H, m), 2.12-2.16 (3H, s), 2.71-2.78 (2H, m), 3.04-3.10 (2H, t, J = 6.3 Hz), |
| | 4.21-4.26 (2H, d, J = 5.7 Hz), 6.09-6.14 (1H, d, J = 5.5 Hz), 6.38-6.43 (1H, d, J = |
| | 8.6 Hz), 6.43-6.48 (1H, t, J = 5.8 Hz), 7.05-7.09 (1H, s), 7.10-7.16 (1H, t, J = 5.8 |
| | Hz), 7.32-7.37 (1H, dd, J = 8.6, 2.4 Hz), 7.79-7.84 (1H, d, J = 5.5 Hz), 7.94-7.98 |
| | (1H, d, J = 2.4 Hz) |
| 1254 | 1.08-1.19 (2H, m), 1.43-1.46 (1H, m), 1.60-1.66 (2H, m), 1.76 (2H, t, J = 11.4 Hz), |
| | 2.11 (3H, s), 2.28 (3H, s), 2.71 (2H, d, J = 11.1 Hz), 3.07 (2H, t, J = 6.3 Hz), 4.45 (2H, |
| | d, J = 5.8 Hz), 6.18 (1H, s), 6.40 (2H, d, J = 7.9 Hz), 7.33 (1H, dd, J = 8.6, 2.4 Hz), 7.48 |
| | (1H, t, J = 5.8 Hz), 7.92 (1H, d, J = 2.4 Hz), 8.02 (1H, s), 11.29 (1H, s) |
| 1255 | 1.13 (2H, qd, J = 12.1, 3.8 Hz), 1.38-1.47 (1H, m), 1.61-1.67 (2H, m), 1.76 (2H, td, |
| | J = 11.6, 2.5 Hz), 2.08-2.12 (6H, m), 2.71 (2H, dd, J = 11.4, 3.5 Hz), 3.05 (2H, t, J = |
| | 6.3 Hz), 4.55 (2H, d, J = 6.5 Hz), 6.06 (1H, t, J = 6.6 Hz), 6.35-6.43 (3H, m), 7.31 (1H, |
| | dd, J = 8.6, 2.4 Hz), 7.61 (1H, s), 7.89 (1H, d, J = 2.4 Hz), 11.84 (1H, s) |
| 1256 | 1.14 (2H, qd, J = 11.9, 3.8 Hz), 1.41-1.50 (1H, m), 1.65 (2H, d, J = 12.8 Hz), 1.74- |
| | 1.85 (2H, m), 2.13 (3H, s), 2.73 (2H, d, J = 10.9 Hz), 3.08 (2H, t, J = 6.3 Hz), 4.21 (2H, |
| | d, J = 5.9 Hz), 6.23 (1H, d, J = 1.3 Hz), 6.39-6.46 (2H, m), 6.53 (1H, t, J = 5.9 Hz), |
| | 6.76 (1H, t, J = 2.6 Hz), 7.10 (1H, s), 7.33-7.40 (2H, m), 7.98 (1H, d, J = 2.4 Hz), |
| | 11.33 (1H, s). |
| 1257 | 1.13 (2H, app qd, J = 12.0, 3.8 Hz), 1.19 (6H, s), 1.39-1.50 (1H, m), 1.60-1.68 (2H, |
| | m), 1.76 (2H, app td, J = 11.6, 2.5 Hz), 2.11 (3H, s), 2.55 (2H, s), 2.68-2.75 (2H, m), |
| | 3.07 (2H, app t, J = 6.3 Hz), 4.09 (2H, d, J = 6.0 Hz), 5.68 (1H, s), 5.84 (1H, d, J = 5.9 |
| | Hz), 5.96 (1H, t, J = 6.1 Hz), 6.38-6.44 (2H, m), 7.29 (1H, dd, J = 8.6, 2.4 Hz), 7.36 |
| | (1H, d, J = 5.9 Hz), 7.88 (1H, d, J = 2.3 Hz). |
| 1258 | 1.14 (2H, qd, J = 12.0, 3.8 Hz), 1.40-1.49 (1H, m), 1.64 (2H, d, J = 12.7 Hz), 1.76 |
| | (2H, td, J = 11.7, 2.5 Hz), 2.11 (3H, s), 2.69-2.75 (2H, m), 3.08 (2H, t, J = 6.3 Hz), |
| | 4.06 (2H, d, J = 5.7 Hz), 6.43 (1H, d, J = 8.6 Hz), 6.45-6.52 (3H, m), 6.82 (1H, t, J = |
| | 5.7 Hz), 7.31 (1H, dd, J = 8.6, 2.4 Hz), 7.92 (1H, d, J = 2.4 Hz), 7.96-8.01 (2H, m). |
| 1259 | 1.13 (2H, qd, J = 12.0, 3.9 Hz), 1.40-1.51 (1H, m), 1.61-1.68 (2H, m), 1.78 (2H, t, |
| | J = 11.5 Hz), 2.12 (3H, s), 2.68-2.76 (2H, m), 3.07 (2H, t, J = 6.3 Hz), 3.73 (3H, s), 4.23 |
| | (2H, d, J = 5.9 Hz), 6.06 (1H, t, J = 6.0 Hz), 6.36-6.43 (2H, m), 6.63 (1H, d, J = 3.1 |
| | Hz), 6.68 (1H, s), 7.01 (1H, s), 7.20 (1H, d, J = 3.1 Hz), 7.30 (1H, s), 7.37 (1H, dd, J = |
| | 8.6, 2.4 Hz), 7.71 (1H, s), 7.97 (1H, d, J = 2.4 Hz). |
| 1260 | 1.08-1.20 (2H, m), 1.39-1.51 (1H, m), 1.64 (2H, dd, J = 13.5, 3.7 Hz), 1.79 (2H, t, |
| | J = 11.1 Hz), 2.13 (3H, s), 2.40-2.48 (2H, m), 2.70-2.77 (2H, m), 3.08 (2H, t, J = 6.3 |
| | Hz), 3.27 (2H, t, J = 6.9 Hz), 3.55 (2H, t, J = 12.8 Hz), 4.19 (2H, d, J = 5.4 Hz), 6.41- |
| | 6.50 (2H, m), 6.65 (1H, d, J = 2.2 Hz), 7.01 (1H, t, J = 5.5 Hz), 7.10 (1H, dd, J = 8.9, |
| | 2.1 Hz), 7.36 (1H, dd, J = 8.6, 2.4 Hz), 7.73 (1H, d, J = 8.8 Hz), 7.90 (1H, s), 7.99 (1H, |
| | d, J = 2.3 Hz), 8.60 (1H, s) |
| 1261 | 1.14 (2H, qd, J = 12.1, 3.8 Hz), 1.41-1.49 (1H, m), 1.61-1.67 (2H, m), 1.72-1.80 |
| | (2H, m), 2.11 (3H, s), 2.69-2.74 (2H, m), 3.08 (2H, t, J = 6.3 Hz), 4.30 (2H, d, J = 5.8 |
| | Hz), 6.44 (1H, d, J = 8.6 Hz), 6.48 (1H, t, J = 5.8 Hz), 6.67 (1H, s), 6.71 (1H, d, J = 3.4 |
| | Hz), 7.33 (1H, d, J = 3.4 Hz), 7.35 (1H, dd, J = 8.6, 2.4 Hz), 7.46 (1H, t, J = 5.9 Hz), |
| | 7.99 (1H, d, J = 2.4 Hz). |
| 1262 | 1.05-1.19 (2H, m), 1.39-1.49 (1H, m), 1.58-1.66 (2H, m), 1.71-1.79 (2H, m), |
| | 2.11 (3H, s), 2.67-2.78 (2H, m), 3.07 (2H, t, J = 6.3 Hz), 4.20 (2H, d, J = 5.7 Hz), 6.27 |
| | (1H, s), 6.42 (2H, d, J = 8.1 Hz), 6.45-6.50 (1H, m), 6.76 (1H, s), 6.83 (1H, s), 7.35 |
| | (1H, dd, J = 8.6, 2.4 Hz), 7.96 (1H, d, J = 2.3 Hz) |
| 1263 | 1.08-1.21 (2H, m), 1.39-1.56 (1H, m), 1.61-1.72 (2H, m), 1.76-1.86 (2H, m), |
| | 2.14 (3H, s), 2.70-2.80 (2H, m), 3.08 (2H, t, J = 6.3 Hz), 4.33 (2H, d, J = 5.7 Hz), 6.44 |
| | (1H, d, J = 8.6 Hz), 6.48 (1H, t, J = 5.8 Hz), 6.69 (1H, d, J = 3.4 Hz), 6.95 (1H, s), 7.28 |
| | (1H, d, J = 3.4 Hz), 7.31-7.48 (2H, m), 7.97 (1H, d, J = 2.4 Hz). |
| 1264 | 1.08-1.19 (2H, m), 1.36-1.52 (1H, m), 1.61-1.67 (2H, m), 1.72-1.81 (2H, m), |
| | 2.11 (3H, s), 2.68-2.74 (2H, m), 3.07 (2H, app t, J = 6.3 Hz), 4.29 (2H, d, J = 5.8 Hz), |
| | 6.40-6.48 (2H, m), 6.65 (1H, dd, J = 3.5, 1.9 Hz), 6.93 (1H, s), 7.15 (1H, t, J = 5.9 |
| | Hz), 7.21 (1H, dd, J = 3.4, 2.4 Hz), 7.30 (1H, d, J = 3.7 Hz), 7.34 (1H, dd, J = 8.6, 2.4 |
| | Hz), 7.63 (1H, d, J = 3.7 Hz), 7.95 (1H, d, J = 2.4 Hz), 11.27 (1H, s) |
| 1265 | 1.59-1.72 (1H, m), 1.99 (2H, tdd, J = 18.4, 7.1, 4.5 Hz), 2.07-2.17 (1H, m), 2.30- |
| | 2.42 (1H, m), 2.97 (1H, dd, J = 10.3, 8.6 Hz), 3.25 (1H, td, J = 9.8, 6.9 Hz), 3.46 (1H, |
| | ddd, J = 10.7, 8.2, 2.7 Hz), 3.62 (1H, dd, J = 10.2, 7.4 Hz), 4.28 (2H, d, J = 5.7 Hz), |
| | 6.17 (1H, tt, J = 56.4, 4.5 Hz), 6.36 (1H, dd, J = 8.7, 0.8 Hz), 6.50 (2H, s), 6.52-6.55 |
| | (1H, m), 6.57 (1H, t, J = 5.9 Hz), 7.12 (1H, t, J = 8.1 Hz), 7.16 (1H, d, J = 5.3 Hz), 7.30 |
| | (1H, d, J = 8.3 Hz), 7.50 (1H, dd, J = 8.7, 2.4 Hz), 7.72 (1H, d, J = 6.1 Hz), 8.09 (1H, d, |
| | J = 2.3 Hz) |
| 1266 | 1.59-1.74 (1H, m), 2.00 (2H, tdd, J = 18.5, 7.2, 4.5 Hz), 2.14 (1H, qd, J = 12.4, 6.7 |
| | Hz), 2.30-2.42 (1H, m), 2.95-3.03 (1H, m), 3.23-3.31 (1H, m), 3.42-3.52 (1H, m), |
| | 3.64 (1H, dd, J = 10.3, 7.4 Hz), 4.22 (2H, d, J = 5.6 Hz), 6.18 (1H, tt, J = 56.4, 4.5 Hz), |
| | 6.40 (1H, d, J = 8.6 Hz), 6.56 (2H, s), 6.71 (1H, d, J = 2.3 Hz), 6.93 (1H, dd, J = 9.1, 2.3 |
| | Hz), 6.97 (1H, t, J = 5.6 Hz), 7.51 (1H, dd, J = 8.6, 2.4 Hz), 7.65 (1H, s), 7.90 (1H, d, |
| | J = 9.0 Hz), 8.10 (1H, d, J = 2.3 Hz) |
| 1267 | 3.11 (2H, t, J = 5.5 Hz), 3.70 (2H, s), 3.96 (2H, t, J = 5.5 Hz), 4.36 (2H, d, J = 5.7 Hz), |
| | 6.60 (2H, s), 6.71 (1H, d, J = 2.4 Hz), 6.92-7.01 (2H, m), 7.10 (1H, t, J = 5.8 Hz), 7.36 |
| | (1H, s), 7.66 (1H, s), 7.82 (1H, dd, J = 8.5, 2.5 Hz), 7.93 (1H, d, J = 9.0 Hz), 8.19 (1H, d) |
| 1268 | 2.85 (1H, br s), 3.01-3.06 (1H, m), 3.25-3.27 (1H, m), 3.92-4.04 (2H, m), 4.30- |
| | 4.34 (2H, m), 4.39 (2H, d, J = 5.8 Hz), 4.42-4.47 (1H, m), 6.14 (1H, d, J = 1.8 Hz), |
| | 6.49 (2H, s), 6.54 (1H, d, J = 7.7 Hz), 6.67 (1H, t, J = 5.8 Hz), 6.81 (1H, d, J = 8.5 Hz), |
| | 7.13 (1H, d, J = 8.1 Hz), 7.17 (1H, d, J = 6.7 Hz), 7.32 (1H, d, J = 8.4 Hz), 7.36 (1H, d, |
| | J = 1.7 Hz), 7.71-7.74 (2H, m), 8.18 (1H, d, J = 2.2 Hz). |
| 1269 | 4.00-4.02 (2H, m), 4.11-4.14 (2H, m), 4.33-4.35 (4H, m), 4.69 (2H, s), 4.76 (1H, t, |
| | J = 5.3 Hz), 6.48 (2H, s), 6.55 (1H, d, J = 7.7 Hz), 6.59-6.62 (1H, m), 6.96 (1H, d, J = |
| | 8.8 Hz), 7.12-7.18 (2H, m), 7.29-7.33 (2H, m), 7.63 (1H, dd, J = 8.7, 2.3 Hz), 7.73 |
| | (1H, d, J = 6.1 Hz), 8.21 (1H, d, J = 2.1 Hz). |
| 1270 | 2.65 (1H, dd, J = 14.0, 3.2 Hz), 3.14 (2H, dd, J = 14.0, 7.1 Hz), 4.16 (2H, s), 4.19 (2H, |
| | d, J = 2.6 Hz), 4.28 (2H, d, J = 5.7 Hz), 4.92 (1H, dd, J = 7.0, 3.1 Hz), 6.51 (1H, d, J = |
| | 8.5 Hz), 6.56 (2H, s), 6.73 (1H, d, J = 2.3 Hz), 6.95 (1H, dd, J = 9.1, 2.3 Hz), 7.01 (1H, |
| | d, J = 1.3 Hz), 7.04 (1H, t, J = 5.8 Hz), 7.36 (1H, d, J = 1.3 Hz), 7.61 (1H, dd, J = 8.5, |
| | 2.3 Hz), 7.66 (1H, s), 7.92 (1H, d, J = 9.1 Hz), 8.18 (1H, d, J = 2.3 Hz) |
| 1271 | 2.90-2.98 (2H, m), 3.73-3.84 (2H, m), 3.88-3.99 (2H, m), 4.02-4.11 (2H, m), |
| | 4.32 (2H, d, J = 5.8 Hz), 6.64 (2H, d, J = 7.0 Hz), 6.69 (1H, d, J = 1.2 Hz), 6.70 (2H, s), |
| | 6.85 (1H, d, J = 8.8 Hz), 7.05 (1H, d, J = 1.3 Hz), 7.15-7.23 (2H, m), 7.34 (1H, d, J = |
| | 8.3 Hz), 7.59 (1H, dd, J = 8.7, 2.4 Hz), 7.72 (1H, d, J = 6.2 Hz), 8.19 (1H, d, J = 2.4 Hz) |
| 1272 | 2.90-2.98 (2H, m), 3.76-3.85 (2H, m), 3.95 (2H, dd, J = 5.8, 2.7 Hz), 4.04-4.10 |
| | (2H, m), 4.25 (2H, d, J = 5.6 Hz), 6.55 (2H, s), 6.65 (1H, d, J = 1.2 Hz), 6.74 (1H, d, J = |
| | 2.3 Hz), 6.90 (1H, d, J = 8.8 Hz), 6.93-7.00 (2H, m), 7.03 (1H, d, J = 1.3 Hz), 7.61 |
| | (1H, dd, J = 8.8, 2.5 Hz), 7.66 (1H, s), 7.92 (1H, d, J = 9.1 Hz), 8.20 (1H, d, J = 2.5 Hz) |
| 1273 | 2.86 (1H, br s), 3.00-3.08 (1H, m), 3.24-3.28 (1H, m), 3.93-4.04 (2H, m), 4.30- |
| | 4.35 (4H, m), 4.44-4.49 (1H, m), 6.15 (1H, s), 6.57 (2H, s), 6.71 (1H, s), 6.86 (1H, d, |
| | J = 8.5 Hz), 6.95 (1H, d, J = 9.0 Hz), 7.04-7.09 (1H, m), 7.36 (1H, d, J = 1.3 Hz), 7.65 |
| | (1H, s), 7.74 (1H, dd, J = 8.5, 2.3 Hz), 7.92 (1H, d, J = 9.1 Hz), 8.20 (1H, d, J = 2.0 Hz) |
| 1274 | 4.05 (2H, t, J = 5.4 Hz), 4.19 (2H, t, J = 5.4 Hz), 4.34 (2H, d, J = 5.8 Hz), 4.90 (2H, s), |
| | 6.48 (2H, s), 6.54 (1H, d, J = 7.7 Hz), 6.61 (1H, t, J = 6.0 Hz), 7.04 (1H, d, J = 8.7 Hz), |
| | 7.13 (1H, t, J = 8.0 Hz), 7.16 (1H, d, J = 6.2 Hz), 7.30 (1H, d, J = 8.3 Hz), 7.35 (1H, t, |
| | J = 51.9 Hz), 7.64 (1H, dd, J = 8.7, 2.4 Hz), 7.73 (1H, d, J = 6.0 Hz), 8.22 (1H, d, J = 2.3 Hz) |
| 1275 | 4.07 (2H, t, J = 5.4 Hz), 4.23 (2H, t, J = 5.4 Hz), 4.35 (2H, d, J = 5.8 Hz), 4.94 (2H, s), |
| | 6.48 (2H, s), 6.54 (1H, d, J = 7.7 Hz), 6.62 (1H, t, J = 6.0 Hz), 7.04 (1H, d, J = 8.7 Hz), |
| | 7.09-7.20 (2H, m), 7.30 (1H, d, J = 8.3 Hz), 7.65 (1H, dd, J = 8.7, 2.4 Hz), 7.73 (1H, |
| | d, J = 6.0 Hz), 8.23 (1H, d, J = 2.3 Hz) |
| 1276 | 3.99 (2H, t, J = 5.3 Hz), 4.11 (2H, t, J = 5.5 Hz), 4.34 (2H, d, J = 5.7 Hz), 4.82 (2H, s), |
| | 6.40-6.58 (3H, m), 6.61 (1H, t, J = 5.9 Hz), 7.01 (1H, d, J = 8.7 Hz), 7.11-7.22 (2H, |
| | m), 7.31 (1H, d, J = 8.2 Hz), 7.63 (1H, dd, J = 8.7, 2.5 Hz), 7.73 (1H, d, J = 6.1 Hz), |
| | 8.21 (1H, d, J = 2.4 Hz), 8.45 (1H, s) |
| 1277 | 4.06 (2H, t, J = 5.5 Hz), 4.19 (2H, t, J = 5.4 Hz), 4.35 (2H, d, J = 6.0 Hz), 4.91 (2H, s), |
| | 6.16 (1H, d, J = 5.8 Hz), 6.54 (1H, s), 7.06 (1H, d, J = 8.7 Hz), 7.10 (1H, t, J = 6.0 Hz), |
| | 7.35 (1H, t, J = 51.9 Hz), 7.62 (1H, dd, J = 8.7, 2.4 Hz), 7.73 (1H, d, J = 5.6 Hz), 8.21 |
| | (1H, d, J = 2.4 Hz), 11.97 (1H, s) |
| 1278 | 2.30 (3H, s), 4.05 (2H, t, J = 5.5 Hz), 4.19 (2H, t, J = 5.5 Hz), 4.34 (2H, d, J = 6.0 Hz), |
| | 4.90 (2H, s), 6.07 (1H, d, J = 5.6 Hz), 6.22 (1H, s), 6.87 (1H, t, J = 6.2 Hz), 7.05 (1H, d, |
| | J = 8.7 Hz), 7.35 (1H, t, J = 51.9 Hz), 7.62 (1H, dd, J = 8.7, 2.4 Hz), 7.65 (1H, d, J = 5.5 |
| | Hz), 8.20 (1H, d, J = 2.3 Hz), 10.97 (1H, s) |
| 1279 | 2.28 (3H, s), 3.94 (2H, t, J = 5.1 Hz), 3.99 (2H, t, J = 5.5 Hz), 4.33 (2H, d, J = 5.8 Hz), |
| | 4.78 (2H, s), 6.49 (2H, s), 6.55 (1H, d, J = 7.7 Hz), 6.61 (1H, t, J = 6.0 Hz), 7.02 (1H, d, |
| | J = 8.7 Hz), 7.10-7.20 (2H, m), 7.30 (1H, d, J = 8.3 Hz), 7.62 (1H, dd, J = 8.7, 2.4 Hz), |
| | 7.73 (1H, d, J = 6.1 Hz), 8.21 (1H, d, J = 2.3 Hz) |
| 1280 | 4.08 (2H, t, J = 5.4 Hz), 4.23 (2H, t, J = 5.4 Hz), 4.35 (2H, d, J = 6.0 Hz), 4.95 (2H, s), |
| | 6.15 (1H, d, J = 5.7 Hz), 6.54 (1H, s), 7.07 (1H, d, J = 8.7 Hz), 7.11 (1H, t, J = 6.0 Hz), |
| | 7.63 (1H, dd, J = 8.7, 2.4 Hz), 7.73 (1H, d, J = 5.6 Hz), 8.21 (1H, d, J = 2.4 Hz), 11.98 |
| | (1H, s) |
| 1281 | 2.64 (3H, d, J = 5.0 Hz), 4.07 (2H, t, J = 5.5 Hz), 4.12 (2H, d, J = 5.9 Hz), 4.20 (2H, t, |
| | J = 5.5 Hz), 4.92 (2H, s), 5.50 (1H, d, J = 2.0 Hz), 5.79-5.90 (2H, m), 6.55 (1H, t, J = |
| | 6.0 Hz), 7.07 (1H, d, J = 8.7 Hz), 7.35 (1H, t, J = 51.9 Hz), 7.50 (1H, d, J = 5.8 Hz), |
| | 7.58 (1H, dd, J = 8.7, 2.4 Hz), 8.15 (1H, d, J = 2.4 Hz) |
| 1282 | 2.34 (3H, s), 4.07 (2H, t, J = 5.4 Hz), 4.22 (2H, t, J = 5.4 Hz), 4.32 (2H, d, J = 5.3 Hz), |
| | 4.94 (2H, s), 6.33 (1H, t, J = 5.4 Hz), 6.49 (2H, s), 6.56 (1H, d, J = 7.7 Hz), 6.98 (1H, s), |
| | 7.14-7.20 (2H, m), 7.33 (1H, d, J = 8.3 Hz), 7.72 (1H, d, J = 6.1 Hz), 8.01 (1H, s) |
| 1283 | 4.09 (2H, t, J = 5.4 Hz), 4.23 (2H, t, J = 5.5 Hz), 4.29 (2H, d, J = 5.8 Hz), 4.96 (2H, s), |
| | 6.67 (1H, d, J = 2.2 Hz), 6.99 (1H, t, J = 5.7 Hz), 7.06-7.12 (2H, m), 7.37 (1H, d, J = |
| | 5.8 Hz), 7.68 (1H, dd, J = 8.7, 2.5 Hz), 7.74 (1H, d, J = 8.9 Hz), 8.16 (1H, d, J = 5.8 Hz), |
| | 8.26 (1H, d, J = 2.1 Hz), 8.85 (1H, s). |
| 1284 | 2.46 (3H, s), 4.06 (2H, t, J = 5.4 Hz), 4.23 (2H, t, J = 5.4 Hz), 4.32 (2H, d, J = 5.4 Hz), |
| | 4.94 (2H, s), 6.41 (1H, d, J = 7.7 Hz), 6.46-6.56 (3H, m), 6.83 (1H, d, J = 8.5 Hz), |
| | 7.13 (1H, t, J = 8.0 Hz), 7.20 (1H, d, J = 6.1 Hz), 7.32 (1H, d, J = 8.3 Hz), 7.42 (1H, d, |
| | J = 8.5 Hz), 7.74 (1H, d, J = 6.1 Hz) |
| 1285 | 1.51 (3H, d, J = 6.8 Hz), 3.52 (1H, ddd, J = 14.9, 11.6, 3.8 Hz), 4.08 (1H, td, J = 11.9, |
| | 4.4 Hz), 4.20-4.27 (1H, m), 4.33 (2H, d, J = 5.8 Hz), 4.68 (1H, dd, J = 14.6, 4.3 Hz), |
| | 5.88 (1H, q, J = 6.8 Hz), 6.48 (2H, s), 6.55 (1H, d, J = 7.7 Hz), 6.61 (1H, t, J = 6.0 Hz), |
| | 7.00 (1H, d, J = 8.8 Hz), 7.10-7.20 (2H, m), 7.30 (1H, d, J = 8.3 Hz), 7.63 (1H, dd, J = |
| | 8.8, 2.4 Hz), 7.73 (1H, d, J = 6.1 Hz), 8.21 (1H, d, J = 2.4 Hz) |
| 1286 | 2.70 (2H, t, J = 5.8 Hz), 3.64 (3H, s), 3.85 (2H, t, J = 5.8 Hz), 4.30 (2H, d, J = 5.8 Hz), |
| | 4.46 (2H, s), 6.47 (2H, s), 6.52-6.59 (2H, m), 6.86 (1H, d, J = 8.8 Hz), 7.10-7.18 |
| | (2H, m), 7.23 (1H, s), 7.30 (1H, d, J = 8.3 Hz), 7.54 (1H, dd, J = 8.8, 2.5 Hz), 7.72 (1H, |
| | d, J = 6.1 Hz), 8.15 (1H, d, J = 2.4 Hz) |
| 1287 | 1.40 (3H, d, J = 6.5 Hz), 3.48 (1H, dd, J = 13.8, 3.4 Hz), 4.36 (2H, d, J = 5.8 Hz), 4.48 |
| | (1H, d, J = 16.9 Hz), 4.56 (1H, d, J = 13.8 Hz), 4.71-4.80 (1H, m), 5.25 (1H, d, J = |
| | 17.0 Hz), 6.48 (2H, s), 6.56 (1H, d, J = 7.7 Hz), 6.63 (1H, t, J = 6.0 Hz), 7.00 (1H, d, J = |
| | 8.7 Hz), 7.11-7.21 (2H, m), 7.31 (1H, d, J = 8.3 Hz), 7.66 (1H, dd, J = 8.7, 2.4 Hz), |
| | 7.73 (1H, d, J = 6.1 Hz), 8.23 (1H, d, J = 2.3 Hz) |
| 1288 | 4.09 (2H, t, J = 5.4 Hz), 4.20 (2H, d, J = 5.8 Hz), 4.24 (2H, t, J = 5.4 Hz), 4.95 (2H, s), |
| | 6.20 (1H, d, J = 2.2 Hz), 6.42 (1H, dd, J = 7.3, 2.3 Hz), 6.61 (1H, t, J = 5.8 Hz), 7.08 |
| | (1H, d, J = 8.6 Hz), 7.15 (1H, d, J = 1.3 Hz), 7.47-7.49 (1H, m), 7.65 (1H, dd, J = 8.8, |
| | 2.4 Hz), 8.12 (1H, dd, J = 7.4, 0.8 Hz), 8.23 (1H, d, J = 2.3 Hz). |
| 1289 | 4.08 (2H, t, J = 5.4 Hz), 4.20-4.26 (4H, m), 4.95 (2H, s), 6.03 (1H, t, J = 6.1 Hz), 6.92 |
| | (1H, d, J = 2.5 Hz), 7.07 (1H, d, J = 8.7 Hz), 7.41 (1H, d, J = 2.8 Hz), 7.68 (1H, dd, J = |
| | 8.7, 2.4 Hz), 7.87 (1H, d, J = 2.6 Hz), 8.23 (1H, d, J = 2.3 Hz), 11.45 (1H, s). |
| 1290 | 1.19 (6H, d, J = 6.2 Hz), 4.09 (2H, t, J = 5.4 Hz), 4.17 (2H, d, J = 5.9 Hz), 4.24 (2H, t, |
| | J = 5.4 Hz), 4.96 (2H, s), 5.12 (1H, hept, J = 6.2 Hz), 5.71 (1H, d, J = 2.0 Hz), 6.20 (1H, |
| | dd, J = 5.8, 2.1 Hz), 6.88 (1H, t, J = 5.9 Hz), 7.08 (1H, d, J = 8.7 Hz), 7.55-7.64 (2H, |
| | m), 8.16 (1H, d, J = 2.3 Hz). |
| 1291 | 1.52 (6H, s), 4.58 (2H, d, J = 6.1 Hz), 5.26 (1H, s), 6.52 (2H, s), 6.55 (1H, d, J = 7.6 |
| | Hz), 6.83 (1H, t, J = 6.0 Hz), 7.14 (1H, t, J = 8.0 Hz), 7.20 (1H, d, J = 6.1 Hz), 7.35 (1H, |
| | d, J = 8.3 Hz), 7.77 (1H, d, J = 6.0 Hz), 8.00-8.09 (2H, m), 8.50 (1H, s), 8.60 (1H, d, |
| | J = 2.0 Hz) |
| 1292 | 4.09 (2H, t, J = 5.4 Hz), 4.23 (2H, t, J = 5.4 Hz), 4.41 (2H, d, J = 5.9 Hz), 4.96 (2H, s), |
| | 6.72 (2H, d, J = 19.7 Hz), 7.08 (1H, d, J = 8.7 Hz), 7.58 (1H, s), 7.65 (1H, dd, J = 8.7, |
| | 2.4 Hz), 8.24 (1H, d, J = 2.4 Hz) |
| 1293 | (Methanol-d4, 500 MHz) 2.38 (3H, s), 4.15 (2H, t, J = 5.4 Hz), 4.34 (2H, t, J = 5.3 Hz), |
| | 4.47 (2H, s), 5.01 (2H, s), 6.12 (1H, s), 6.42 (1H, s), 7.04 (1H, d, J = 8.7 Hz), 7.71 (1H, |
| | dd, J = 8.8, 2.4 Hz), 8.25 (1H, d, J = 2.4 Hz) |
| 1294 | 1.51 (3H, d, J = 6.8 Hz), 3.52 (1H, ddd, J = 14.9, 11.6, 3.8 Hz), 4.08 (1H, td, J = 11.9, |
| | 4.4 Hz), 4.20-4.27 (1H, m), 4.33 (2H, d, J = 5.8 Hz), 4.68 (1H, dd, J = 14.6, 4.3 Hz), |
| | 5.88 (1H, q, J = 6.8 Hz), 6.48 (2H, s), 6.55 (1H, d, J = 7.7 Hz), 6.61 (1H, t, J = 6.0 Hz), |
| | 7.00 (1H, d, J = 8.8 Hz), 7.10-7.20 (2H, m), 7.30 (1H, d, J = 8.3 Hz), 7.63 (1H, dd, J = |
| | 8.8, 2.4 Hz), 7.73 (1H, d, J = 6.1 Hz), 8.21 (1H, d, J = 2.4 Hz) |
| 1295 | 1.51 (3H, d, J = 6.8 Hz), 3.52 (1H, ddd, J = 14.9, 11.6, 3.8 Hz), 4.08 (1H, td, J = 11.9, |
| | 4.4 Hz), 4.20-4.27 (1H, m), 4.33 (2H, d, J = 5.8 Hz), 4.68 (1H, dd, J = 14.6, 4.3 Hz), |
| | 5.88 (1H, q, J = 6.8 Hz), 6.48 (2H, s), 6.55 (1H, d, J = 7.7 Hz), 6.61 (1H, t, J = 6.0 Hz), |
| | 7.00 (1H, d, J = 8.8 Hz), 7.10-7.20 (2H, m), 7.30 (1H, d, J = 8.3 Hz), 7.63 (1H, dd, J = |
| | 8.8, 2.4 Hz), 7.73 (1H, d, J = 6.1 Hz), 8.21 (1H, d, J = 2.4 Hz) |
| 1296 | 1.40 (3H, d, J = 6.5 Hz), 3.48 (1H, dd, J = 13.8, 3.4 Hz), 4.36 (2H, d, J = 5.8 Hz), 4.48 |
| | (1H, d, J = 16.9 Hz), 4.56 (1H, d, J = 13.8 Hz), 4.71-4.80 (1H, m), 5.25 (1H, d, J = |
| | 17.0 Hz), 6.48 (2H, s), 6.56 (1H, d, J = 7.7 Hz), 6.63 (1H, t, J = 6.0 Hz), 7.00 (1H, d, J = |
| | 8.7 Hz), 7.11-7.21 (2H, m), 7.31 (1H, d, J = 8.3 Hz), 7.66 (1H, dd, J = 8.7, 2.4 Hz), |
| | 7.73 (1H, d, J = 6.1 Hz), 8.23 (1H, d, J = 2.3 Hz) |
| 1297 | 1.40 (3H, d, J = 6.5 Hz), 3.48 (1H, dd, J = 13.8, 3.4 Hz), 4.36 (2H, d, J = 5.8 Hz), 4.48 |
| | (1H, d, J = 16.9 Hz), 4.56 (1H, d, J = 13.8 Hz), 4.71-4.80 (1H, m), 5.25 (1H, d, J = |
| | 17.0 Hz), 6.48 (2H, s), 6.56 (1H, d, J = 7.7 Hz), 6.63 (1H, t, J = 6.0 Hz), 7.00 (1H, d, J = |
| | 8.7 Hz), 7.11-7.21 (2H, m), 7.31 (1H, d, J = 8.3 Hz), 7.66 (1H, dd, J = 8.7, 2.4 Hz), |
| | 7.73 (1H, d, J = 6.1 Hz), 8.23 (1H, d, J = 2.3 Hz) |
| 1298 | 4.12 (t, J = 5.4 Hz, 2H), 4.27 (t, J = 5.4 Hz, 2H), 4.51 (d, J = 5.6 Hz, 2H), 5.01 (s, 2H), |
| | 6.31 (d, J = 7.7 Hz, 1H), 6.55 (s, 2H), 6.76 (t, J = 5.8 Hz, 1H), 7.13 (t, J = 8.0 Hz, 1H), |
| | 7.20 (d, J = 6.3 Hz, 1H), 7.29 (d, J = 8.9 Hz, 1H), 7.35 (d, J = 8.3 Hz, 1H), 7.71 (d, J = |
| | 8.9 Hz, 1H), 7.77 (d, J = 6.1 Hz, 1H) |
| 1299 | 4.10 (2H, t, J = 5.5 Hz), 4.23 (2H, t, J = 5.4 Hz), 4.42 (2H, d, J = 5.6 Hz), 4.99 (2H, s), |
| | 6.54 (1H, d, J = 7.8 Hz), 6.59 (1H, t, J = 5.6 Hz), 6.76 (2H, br s), 7.16-7.24 (2H, m), |
| | 7.28 (1H, s), 7.39 (1H, d, J = 8.3 Hz), 7.73 (1H, d, J = 6.2 Hz), 8.10 (1H, s) |
| 1300 | 3.73 (2H, t, J = 5.5 Hz), 4.24 (2H, t, J = 5.4 Hz), 4.37 (2H, d, J = 5.9 Hz), 4.61 (2H, s), |
| | 6.44-6.49 (3H, m), 6.68 (1H, t, J = 6.0 Hz), 7.03-7.08 (2H, m), 7.10 (1H, t, J = 8.0 |
| | Hz), 7.19 (1H, d, J = 6.1 Hz), 7.25-7.32 (3H, m), 7.73 (1H, d, J = 6.0 Hz) |
| 1301 | 4.16 (t, J = 5.5 Hz, 2H), 4.25 (t, J = 5.4 Hz, 2H), 4.49 (d, J = 5.4 Hz, 2H), 5.06 (s, 2H), |
| | 6.39 (d, J = 7.7 Hz, 1H), 6.54 (s, 2H), 6.65 (t, J = 5.7 Hz, 1H), 7.12-7.21 (m, 2H), 7.34- |
| | 7.39 (m, 2H), 7.76 (d, J = 6.1 Hz, 1H), 8.26 (s, 1H) |
| 1302 | 2.28 (3H, s), 3.57 (2H, t, J = 5.4 Hz), 4.20 (2H, t, J = 5.4 Hz), 4.38 (2H, d, J = 5.8 Hz), |
| | 4.55 (2H, s), 6.51 (2H, s), 6.55 (1H, d, J = 7.7 Hz), 6.66 (1H, t, J = 6.0 Hz), 7.14 (1H, t, |
| | J = 8.0 Hz), 7.17 (1H, d, J = 6.2 Hz), 7.32 (1H, d, J = 8.3 Hz), 7.61 (1H, d, J = 2.3 Hz), |
| | 7.74 (1H, d, J = 6.1 Hz), 8.16 (1H, d, J = 2.3 Hz). |
| 1303 | 1.51 (3H, d, J = 6.8 Hz), 2.34 (3H, s), 3.46-3.56 (1H, m), 4.06-4.12 (1H, m), 4.20- |
| | 4.26 (1H, m), 4.31 (2H, d, J = 5.1 Hz), 4.68 (1H, dd, J = 14.5, 4.3 Hz), 5.89 (1H, q, J = |
| | 6.8 Hz), 6.31 (1H, t, J = 5.4 Hz), 6.48 (2H, s), 6.57 (1H, d, J = 7.7 Hz), 6.93 (1H, s), |
| | 7.14-7.20 (2H, m), 7.33 (1H, d, J = 8.3 Hz), 7.71 (1H, d, J = 6.1 Hz), 7.99 (1H, s) |
| 1304 | 1.51 (3H, d, J = 6.8 Hz), 2.34 (3H, s), 3.51 (1H, ddd, J = 15.0, 11.7, 3.9 Hz), 4.05- |
| | 4.12 (1H, m), 4.23 (1H, dd, J = 12.2, 3.6 Hz), 4.31 (2H, d, J = 5.3 Hz), 4.68 (1H, dd, J = |
| | 14.5, 4.3 Hz), 5.89 (1H, q, J = 6.8 Hz), 6.31 (1H, t, J = 5.4 Hz), 6.49 (2H, s), 6.57 (1H, |
| | d, J = 7.7 Hz), 6.93 (1H, s), 7.13-7.21 (2H, m), 7.33 (1H, d, J = 8.3 Hz), 7.71 (1H, d, J = |
| | 6.1 Hz), 7.99 (1H, s) |
| 1305 | 1.51 (3H, d, J = 6.8 Hz), 2.34 (3H, s), 3.51 (1H, ddd, J = 15.0, 11.6, 3.8 Hz), 4.08 (1H, |
| | td, J = 11.9, 4.4 Hz), 4.23 (1H, dd, J = 12.4, 3.6 Hz), 4.31 (2H, d, J = 5.3 Hz), 4.68 (1H, |
| | dd, J = 14.6, 4.3 Hz), 5.89 (1H, q, J = 6.8 Hz), 6.31 (1H, t, J = 5.5 Hz), 6.49 (2H, s), |
| | 6.57 (1H, d, J = 7.7 Hz), 6.93 (1H, s), 7.13-7.21 (2H, m), 7.33 (1H, d, J = 8.3 Hz), |
| | 7.71 (1H, d, J = 6.1 Hz), 7.99 (1H, s) |
| 1306 | 3.66 (2H, t, J = 5.4 Hz), 4.20 (2H, t, J = 5.4 Hz), 4.51 (2H, d, J = 5.7 Hz), 4.62 (2H, s), |
| | 6.52 (2H, d, J = 5.3 Hz), 6.59 (1H, d, J = 7.6 Hz), 6.74 (1H, t, J = 6.1 Hz), 7.13-7.20 |
| | (2H, m), 7.35 (1H, d, J = 8.3 Hz), 7.76 (1H, d, J = 6.0 Hz), 8.18 (1H, d, J = 2.2 Hz), 8.61 |
| | (1H, d, J = 2.2 Hz) |
| 1307 | 0.84-0.92 (2H, m), 0.94-1.02 (2H, m), 2.09-2.18 (1H, m), 4.06 (2H, t, J = 5.4 Hz), |
| | 4.20 (2H, t, J = 5.4 Hz), 4.47 (2H, d, J = 5.2 Hz), 4.94 (2H, s), 6.39 (1H, t, J = 5.5 Hz), |
| | 6.50-6.62 (4H, m), 7.15-7.21 (2H, m), 7.33 (1H, d, J = 8.3 Hz), 7.72 (1H, d, J = 6.1 |
| | Hz), 7.99 (1H, s) |
| 1308 | 1.23 (6H, d, J = 6.8 Hz), 3.21-3.28 (1H, m), 4.09 (2H, t, J = 5.4 Hz), 4.24 (2H, t, J = |
| | 5.4 Hz), 4.34 (2H, d, J = 5.1 Hz), 4.97 (2H, s), 6.28 (1H, t, J = 5.4 Hz), 6.49 (2H, s), |
| | 6.59 (1H, d, J = 7.7 Hz), 7.00 (1H, s), 7.15 (1H, d, J = 6.1 Hz), 7.18 (1H, t, J = 8.0 Hz), |
| | 7.33 (1H, d, J = 8.3 Hz), 7.70 (1H, d, J = 6.1 Hz), 8.07 (1H, s) |
| 1309 | 1.21 (3H, t, J = 7.5 Hz), 2.71 (2H, q, J = 7.5 Hz), 4.08 (2H, t, J = 5.4 Hz), 4.23 (2H, t, J = |
| | 5.4 Hz), 4.34 (2H, d, J = 5.4 Hz), 4.95 (2H, s), 6.34 (1H, t, J = 5.4 Hz), 6.49 (2H, s), |
| | 6.55 (1H, d, J = 7.6 Hz), 6.95 (1H, s), 7.14-7.20 (2H, m), 7.33 (1H, d, J = 8.3 Hz), |
| | 7.71 (1H, d, J = 6.1 Hz), 8.04 (1H, s) |
| 1310 | 2.28 (3H, s), 2.95-3.04 (2H, m), 3.73-3.85 (2H, m), 3.91-4.02 (4H, m), 4.32 (2H, |
| | d, J = 5.7 Hz), 6.51 (2H, s), 6.56-6.63 (2H, m), 6.84 (1H, d, J = 8.7 Hz), 7.12-7.20 |
| | (2H, m), 7.31 (1H, d, J = 8.3 Hz), 7.60 (1H, dd, J = 8.8, 2.4 Hz), 7.72 (1H, d, J = 6.1 |
| | Hz), 8.19 (1H, d, J = 2.4 Hz) |
| 1311 | 1.05 (3H, d, J = 6.8 Hz), 4.21 (1H, d, J = 12.6 Hz), 4.29-4.34 (1H, m), 4.36 (2H, d, J = |
| | 5.9 Hz), 4.43 (1H, d, J = 17.3 Hz), 5.20-5.27 (2H, m), 6.48 (2H, s), 6.56 (1H, d, J = |
| | 7.7 Hz), 6.63 (1H, t, J = 6.0 Hz), 6.97 (1H, d, J = 8.7 Hz), 7.14 (1H, t, J = 8.0 Hz), 7.17 |
| | (1H, d, J = 6.1 Hz), 7.31 (1H, d, J = 8.3 Hz), 7.66 (1H, dd, J = 8.7, 2.4 Hz), 7.73 (1H, d, |
| | J = 6.1 Hz), 8.26 (1H, d, J = 2.4 Hz). |
| 1312 | 3.07 (2H, t, J = 5.5 Hz), 3.67 (2H, s), 3.93 (2H, t, J = 5.5 Hz), 4.40 (2H, d, J = 5.8 Hz), |
| | 6.50-6.57 (3H, m), 6.69 (1H, t, J = 5.9 Hz), 6.93 (1H, d, J = 8.5, 0.7 Hz), 7.10-7.20 |
| | (2H, m), 7.32 (1H, d, J = 8.3 Hz), 7.35 (1H, s), 7.73 (1H, d, J = 6.1 Hz), 7.79 (1H, dd, |
| | J = 8.5, 2.5 Hz), 8.16 (1H, d) |
| 1313 | 2.31 (3H, s), 4.08 (2H, t, J = 5.4 Hz), 4.23 (2H, t, J = 5.4 Hz), 4.33 (2H, d, J = 5.3 Hz), |
| | 4.96 (2H, s), 6.18 (1H, d, J = 5.7 Hz), 6.58 (1H, s), 6.84 (1H, t, J = 5.4 Hz), 6.99 (1H, s), |
| | 7.76 (1H, d, J = 5.6 Hz), 8.04 (1H, s), 11.98 (1H, s) |
| 1314 | 1.52 (3H, d, J = 6.8 Hz), 2.30 (3H, s), 3.52 (1H, ddd, J = 15.0, 11.6, 3.8 Hz), 4.08 (1H, |
| | td, J = 12.0, 4.4 Hz), 4.24 (1H, dd, J = 12.3, 3.6 Hz), 4.32 (2H, d, J = 5.3 Hz), 4.70 (1H, |
| | dd, J = 14.5, 4.3 Hz), 5.90 (1H, q, J = 6.8 Hz), 6.19 (1H, d, J = 5.7 Hz), 6.59 (1H, s), |
| | 6.82 (1H, t, J = 5.4 Hz), 6.95 (1H, s), 7.76 (1H, d, J = 5.6 Hz), 8.02 (1H, s), 11.97 (1H, s) |
| 1315 | 1.52 (3H, d, J = 6.8 Hz), 2.30 (3H, s), 3.52 (1H, ddd, J = 15.0, 11.7, 3.8 Hz), 4.08 (1H, |
| | td, J = 12.0, 4.4 Hz), 4.24 (1H, dd, J = 11.8, 3.5 Hz), 4.32 (2H, d, J = 5.4 Hz), 4.70 (1H, |
| | dd, J = 14.5, 4.3 Hz), 5.90 (1H, q, J = 6.8 Hz), 6.19 (1H, d, J = 5.7 Hz), 6.58 (1H, s), |
| | 6.81 (1H, s), 6.95 (1H, s), 7.76 (1H, d, J = 5.6 Hz), 8.02 (1H, s), 11.96 (1H, s) |
| 1316 | 1.52 (3H, d, J = 6.8 Hz), 2.30 (3H, s), 3.52 (1H, ddd, J = 15.0, 11.6, 3.8 Hz), 4.08 (1H, |
| | td, J = 12.0, 4.4 Hz), 4.24 (1H, d, J = 12.2 Hz), 4.32 (2H, d, J = 5.3 Hz), 4.70 (1H, dd, |
| | J = 14.5, 4.3 Hz), 5.90 (1H, q, J = 6.7 Hz), 6.19 (1H, d, J = 5.7 Hz), 6.58 (1H, s), 6.77- |
| | 6.85 (1H, m), 6.95 (1H, s), 7.76 (1H, d, J = 5.6 Hz), 8.02 (1H, s), 11.92 (1H, s) |
| 1317 | 4.08 (2H, t, J = 5.4 Hz), 4.23 (2H, t, J = 5.4 Hz), 4.39 (2H, d, J = 6.0 Hz), 4.95 (2H, s), |
| | 6.68 (1H, t, J = 6.1 Hz), 7.00 (2H, s), 7.05-7.10 (1H, m), 7.14 (1H, dd, J = 6.2, 0.9 |
| | Hz), 7.68 (1H, dd, J = 8.7, 2.4 Hz), 7.75 (1H, s), 7.95 (1H, d, J = 6.0 Hz), 8.26 (1H, d, |
| | J = 2.3 Hz), 8.74 (1H, d, J = 0.8 Hz). |
| 1318 | 4.06 (2H, t, J = 5.4 Hz), 4.22 (2H, t, J = 5.4 Hz), 4.56 (2H, d, J = 5.6 Hz), 4.94 (2H, s), |
| | 6.71 (2H, s), 7.04 (1H, dd, J = 8.8, 0.8 Hz), 7.09 (1H, dd, J = 6.0, 0.9 Hz), 7.15 (1H, dd, |
| | J = 6.1, 0.9 Hz), 7.65 (1H, dd, J = 8.7, 2.4 Hz), 7.72 (1H, t, J = 5.9 Hz), 7.84 (1H, d, J = |
| | 6.0 Hz), 7.86 (1H, d, J = 5.9 Hz), 8.20 (1H, dd, J = 2.4, 0.7 Hz) |
| 1319 | 2.33 (3H, s), 3.24 (3H, s), 3.70 (1H, ddd, J = 15.0, 11.6, 3.8 Hz), 3.85 (1H, dd, J = |
| | 10.3, 3.5 Hz), 3.96 (1H, dd, J = 10.3, 5.6 Hz), 4.03-4.12 (1H, m), 4.21-4.28 (1H, m), |
| | 4.31 (2H, d, J = 5.3 Hz), 4.66 (1H, dd, J = 14.5, 4.2 Hz), 5.99-6.05 (1H, m), 6.32 (1H, |
| | t, J = 5.5 Hz), 6.51 (2H, s), 6.57 (1H, d, J = 7.7 Hz), 6.94 (1H, s), 7.14-7.21 (2H, m), |
| | 7.33 (1H, d, J = 8.3 Hz), 7.71 (1H, d, J = 6.1 Hz), 7.97 (1H, s) |
| 1320 | 1.03 (3H, t, J = 7.4 Hz), 1.90-2.02 (2H, m), 2.33 (3H, s), 3.57 (1H, ddd, J = 15.2, |
| | 11.4, 4.2 Hz), 4.09 (1H, td, J = 12.0, 4.4 Hz), 4.18 (1H, dd, J = 12.2, 3.9 Hz), 4.29 (2H, |
| | d, J = 5.3 Hz), 4.60 (1H, dd, J = 14.7, 4.3 Hz), 5.78 (1H, t, J = 7.4 Hz), 6.29 (1H, t, J = |
| | 5.4 Hz), 6.48 (2H, s), 6.57 (1H, d, J = 7.7 Hz), 6.95 (1H, s), 7.13-7.21 (2H, m), 7.32 |
| | (1H, d, J = 8.3 Hz), 7.71 (1H, d, J = 6.1 Hz), 7.97 (1H, s) |
| 1321 | 1.03 (3H, t, J = 7.4 Hz), 1.90-2.02 (2H, m), 2.33 (3H, s), 3.57 (1H, ddd, J = 15.2, |
| | 11.4, 4.2 Hz), 4.09 (1H, td, J = 12.0, 4.4 Hz), 4.18 (1H, dd, J = 12.2, 3.9 Hz), 4.29 (2H, |
| | d, J = 5.3 Hz), 4.60 (1H, dd, J = 14.7, 4.3 Hz), 5.78 (1H, t, J = 7.4 Hz), 6.29 (1H, t, J = |
| | 5.4 Hz), 6.48 (2H, s), 6.57 (1H, d, J = 7.7 Hz), 6.95 (1H, s), 7.13-7.21 (2H, m), 7.32 |
| | (1H, d, J = 8.3 Hz), 7.71 (1H, d, J = 6.1 Hz), 7.97 (1H, s) |
| 1322 | 2.33 (3H, s), 4.04 (2H, d, J = 5.4 Hz), 4.09 (2H, t, J = 5.5 Hz), 4.23 (2H, t, J = 5.4 Hz), |
| | 4.97 (2H, s), 5.69 (1H, t, J = 5.4 Hz), 6.89 (1H, dd, J = 9.6, 2.2 Hz), 7.01 (1H, s), 7.32 |
| | (1H, d, J = 9.6 Hz), 7.34 (1H, d, J = 1.0 Hz), 7.64 (1H, d, J = 2.1 Hz), 7.69 (1H, s), 8.10 |
| | (1H, s) |
| 1323 | 2.28 (3H, s), 4.04-4.13 (4H, m), 4.23 (2H, t, J = 5.4 Hz), 4.96 (2H, s), 5.48 (2H, s), |
| | 5.60 (1H, d, J = 2.1 Hz), 5.91 (1H, dd, J = 6.0, 2.0 Hz), 6.48 (1H, s), 6.98 (1H, s), 7.44 |
| | (1H, d, J = 5.9 Hz), 8.00 (1H, s) |
| 1324 | 1.20-1.31 (2H, m), 1.63-1.72 (3H, m), 1.79-1.87 (2H, m), 2.14 (3H, s), 2.72-2.79 |
| | (2H, m), 4.08 (2H, d, J = 6.1 Hz), 4.36 (2H, d, J = 5.6 Hz), 6.80 (1H, d, J = 8.5 Hz), 6.86 |
| | (1H, d, J = 2.3 Hz), 7.36 (1H, dd, J = 9.1, 2.3 Hz), 7.50 (1H, t, J = 5.8 Hz), 7.70-7.76 |
| | (2H, m), 7.92 (1H, d, J = 9.1 Hz), 8.21 (1H, d, J = 2.4 Hz), 8.33 (1H, d, J = 5.7 Hz) |
| 1325 | 1.20-1.31 (2H, m), 1.64-1.72 (3H, m), 1.78-1.87 (2H, m), 2.13 (3H, s), 2.71-2.78 |
| | (2H, m), 4.07 (2H, d, J = 6.1 Hz), 4.31 (2H, d, J = 5.9 Hz), 6.72 (1H, t, J = 5.8 Hz), 6.76- |
| | 6.80 (1H, m), 6.84 (1H, d, J = 2.3 Hz), 7.17 (1H, dd, J = 9.1, 2.4 Hz), 7.22-7.29 (2H, |
| | m), 7.54 (1H, d, J = 7.8 Hz), 7.72 (1H, dd, J = 8.5, 2.5 Hz), 7.88 (1H, d, J = 9.1 Hz), |
| | 8.20 (1H, d, J = 2.4 Hz) |
| 1326 | 1.21-1.32 (2H, m), 1.60-1.74 (3H, m), 1.80-1.90 (2H, m), 2.15 (3H, s), 2.70 (3H, |
| | s), 2.73-2.81 (2H, m), 4.07 (2H, d, J = 6.1 Hz), 4.32 (2H, d, J = 5.7 Hz), 6.68 (1H, d, |
| | J = 2.3 Hz), 6.78 (1H, d, J = 8.5 Hz), 6.94 (1H, t, J = 5.9 Hz), 7.09 (1H, dd, J = 9.1, 2.4 |
| | Hz), 7.24 (1H, d, J = 5.8 Hz), 7.71 (1H, dd, J = 8.5, 2.5 Hz), 7.87 (1H, d, J = 9.1 Hz), |
| | 8.03 (1H, d, J = 5.8 Hz), 8.19 (1H, d, J = 2.4 Hz) |
| 1327 | 1.20-1.30 (2H, m), 1.63-1.72 (3H, m), 1.79-1.85 (2H, m), 2.13 (3H, s), 2.71-2.77 |
| | (2H, m), 4.07 (2H, d, J = 6.1 Hz), 4.57 (2H, d, J = 5.6 Hz), 6.77 (1H, d, J = 8.5 Hz), 6.88 |
| | (1H, d, J = 9.0 Hz), 7.36 (1H, d, J = 5.9 Hz), 7.73 (1H, dd, J = 8.5, 2.5 Hz), 7.98 (1H, d, |
| | J = 9.0 Hz), 8.03 (1H, t, J = 5.7 Hz), 8.19 (1H, d, J = 2.4 Hz), 8.39 (1H, d, J = 5.8 Hz), |
| | 8.84 (1H, s) |
| 1328 | 1.43-1.57 (2H, m), 1.89-1.97 (2H, m), 1.97-2.05 (1H, m), 2.71-2.81 (3H, m), |
| | 2.91-3.02 (2H, m), 3.42-3.48 (2H, m), 3.92 (3H, s), 4.13 (2H, d, J = 6.3 Hz), 4.43 |
| | (2H, d, J = 5.6 Hz), 6.83 (1H, d, J = 8.5 Hz), 7.32 (1H, dd, J = 9.0, 2.2 Hz), 7.71-7.75 |
| | (1H, m), 7.77 (1H, dd, J = 8.5, 2.5 Hz), 8.01 (1H, d, J = 9.0 Hz), 8.08-8.13 (1H, m), |
| | 8.24 (1H, d, J = 2.5 Hz), 8.80 (1H, s), 9.13 (1H, s), 9.29 (1H, s). |
| 1329 | 1.55-1.68 (2H, m), 1.84-2.06 (3H, m), 2.66-2.71 (3H, m), 2.87-2.99 (2H, m), |
| | 3.33-3.43 (2H, m), 4.12 (2H, d, J = 6.4 Hz), 4.42-4.53 (2H, m), 6.83 (1H, d, J = 8.5 |
| | Hz), 7.48 (1H, d, J = 9.3 Hz), 7.78 (1H, dd, J = 8.6, 2.5 Hz), 7.92-8.03 (1H, m), 8.12- |
| | 8.22 (1H, m), 8.24 (1H, d, J = 2.5 Hz), 8.71 (1H, s), 8.96 (1H, s), 9.27 (1H, s). 1 × |
| | exchangeable proton (COOH). |
| 1330 | 1.23-1.35 (2H, m), 1.72 (3H, m), 1.96-2.06 (2H, m), 2.24 (3H, s), 2.81-2.89 (2H, |
| | m), 4.09 (2H, d, J = 6.1 Hz), 4.30 (2H, d, J = 5.6 Hz), 6.78 (1H, d, J = 8.5 Hz), 7.14 (1H, |
| | dd, J = 8.9, 2.2 Hz), 7.19 (1H, t, J = 5.8 Hz), 7.30 (1H, d, J = 2.2 Hz), 7.46 (1H, s), 7.71 |
| | (1H, dd, J = 8.5, 2.5 Hz), 7.81 (1H, d, J = 8.9 Hz), 7.94 (1H, s), 8.17 (1H, d, J = 2.4 Hz), |
| | 8.39 (1H, s), 8.91 (1H, s). |
| 1331 | 1.03 (3H, t, J = 7.4 Hz), 1.90-2.02 (2H, m), 2.33 (3H, s), 3.57 (1H, ddd, J = 15.2, 11.4, 4.2 |
| | Hz), 4.09 (1H, td, J = 12.0, 4.4 Hz), 4.18 (1H, dd, J = 12.2, 3.9 Hz), 4.29 (2H, d, J = 5.3 Hz), |
| | 4.60 (1H, dd, J = 14.7, 4.3 Hz), 5.78 (1H, t, J = 7.4 Hz), 6.29 (1H, t, J = 5.4 Hz), 6.48 (2H, s), |
| | 6.57 (1H, d, J = 7.7 Hz), 6.95 (1H, s), 7.13-7.21 (2H, m), 7.32 (1H, d, J = 8.3 Hz), 7.71 (1H, d, |
| | J = 6.1 Hz), 7.97 (1H, s) |
| 1332 | 2.67 (3H, s), 4.08 (2H, t, J = 5.4 Hz), 4.14 (2H, s), 4.23 (2H, t, J = 5.4 Hz), 4.94 (2H, s), 7.03 |
| | (1H, d, J = 8.8 Hz), 7.24 (1H, d, J = 6.3 Hz), 7.30 (1H, d, J = 7.5 Hz), 7.42 (1H, t, J = 7.9 Hz), |
| | 7.53 (1H, dd, J = 8.7, 2.4 Hz), 7.77 (1H, d, J = 6.3 Hz), 7.90-7.94 (1H, m), 8.09 (1H, d, J = 2.3 Hz) |
| 1333 | 2.33 (3H, s), 4.08 (2H, t, J = 5.4 Hz), 4.19-4.25 (4H, m), 4.96 (2H, s), 6.28 (2H, s), 6.40 (1H, t, |
| | J = 5.3 Hz), 6.55 (1H, d, J = 2.3 Hz), 6.59 (1H, d, J = 5.8 Hz), 6.87 (1H, dd, J = 9.0, 2.3 Hz), 7.00 |
| | (1H, s), 7.55 (1H, d, J = 5.8 Hz), 7.84 (1H, d, J = 9.0 Hz), 8.08 (1H, s) |
| 1334 | 2.34 (3H, s), 4.07-4.11 (2H, m), 4.23 (2H, t, J = 5.4 Hz), 4.26 (2H, d, J = 5.2 Hz), 4.96 (2H, s), |
| | 6.54 (2H, s), 6.73 (1H, d, J = 2.3 Hz), 6.79 (1H, t, J = 5.2 Hz), 6.95 (1H, dd, J = 9.1, 2.4 Hz), |
| | 7.01 (1H, s), 7.66 (1H, s), 7.91 (1H, d, J = 9.1 Hz), 8.10 (1H, s). |
| 1335 | (Methanol-d4) 3.74 (2H, t, J = 5.5 Hz), 4.30 (2H, t, J = 5.5 Hz), 4.56 (2H, s), 4.68-4.71 (4H, |
| | m), 6.69 (1H, d, J = 7.7 Hz), 7.21-7.29 (2H, m), 7.34 (1H, d, J = 8.4 Hz), 7.73 (1H, d, J = 6.3 |
| | Hz), 7.97 (1H, d, J = 2.3 Hz), 8.29 (1H, d, J = 2.3 Hz). |
| 1336 | (Methanol-d4) 1.34 (3H, t, J = 7.5 Hz), 2.87 (2H, q, J = 7.5 Hz), 4.16 (2H, t, J = 5.5 Hz), 4.33 |
| | (2H, t, J = 5.4 Hz), 4.45 (2H, s), 5.04 (2H, s), 6.62 (1H, dd, J = 7.6, 1.1 Hz), 6.79 (1H, d, J = 8.5 |
| | Hz), 7.20 (1H, dd, J = 6.4, 0.9 Hz), 7.24-7.38 (2H, m), 7.59 (1H, d, J = 8.5 Hz), 7.72 (1H, d, J = |
| | 6.3 Hz). |
| 1337 | 4.09 (2H, t, J = 5.4 Hz), 4.21 (2H, t, J = 5.4 Hz), 4.95 (2H, s), 6.15 (2H, s), 6.93-7.00 |
| | (2H, m), 7.09 (1H, d, J = 7.7 Hz), 7.16 (1H, d, J = 8.2 Hz), 7.43 (1H, d, J = 5.9 Hz), 7.57 |
| | (1H, d, J = 5.9 Hz), 7.81 (1H, dd, J = 8.9, 2.4 Hz), 8.43 (1H, d, J = 2.4 Hz) |
| 1338 | 4.08 (2H, t, J = 5.5 Hz), 4.23 (2H, t, J = 5.4 Hz), 4.35 (2H, d, J = 6.0 Hz), 4.94 (2H, s), |
| | 6.23 (1H, d, J = 1.2 Hz), 6.68 (1H, t, J = 6.1 Hz), 6.76 (1H, app t, J = 2.5 Hz), 7.06 (1H, |
| | d, J = 8.7 Hz), 7.10 (1H, d, J = 1.4 Hz), 7.38 (1H, app t, J = 2.8 Hz), 7.66 (1H, dd, J = |
| | 8.7, 2.4 Hz), 8.24 (1H, d, J = 2.3 Hz), 11.35 (1H, s). |
| 1339 | 2.29 (3H, s), 2.77 (2H, t, J = 8.6 Hz), 3.35-3.42 (2H, m), 4.07 (2H, t, J = 5.4 Hz), 4.20- |
| | 4.25 (4H, m), 4.95 (2H, s), 5.72 (1H, s), 5.88 (1H, d, J = 6.0 Hz), 6.00 (1H, t, J = 5.8 |
| | Hz), 6.96 (1H, s), 7.39 (1H, d, J = 5.9 Hz), 7.97 (1H, s) |
| 1341 | 2.33 (3H, s), 2.36 (3H, s), 4.07 (2H, t, J = 5.5 Hz), 4.23 (2H, t, J = 5.4 Hz), 4.31 (2H, d, |
| | J = 5.6 Hz), 4.94 (2H, s), 5.78 (1H, t, J = 5.6 Hz), 6.35 (1H, t, J = 0.9 Hz), 6.98 (1H, s), |
| | 7.30 (1H, s), 7.96 (1H, s), 8.04 (1H, s), 8.49 (1H, s) |
| 1342 | 1.06 (3H, d, J = 6.8 Hz), 2.36 (3H, s), 4.21 (1H, d, J = 12.6 Hz), 4.27-4.37 (3H, m), |
| | 4.42 (1H, d, J = 17.3 Hz), 5.19-5.31 (2H, m), 6.35 (1H, t, J = 5.4 Hz), 6.50 (2H, |
| | s), 6.57 (1H, d, J = 7.7 Hz), 6.90 (1H, s), 7.14-7.23 (2H, m), 7.33 (1H, d, J = 8.3 Hz), |
| | 7.72 (1H, d, J = 6.1 Hz), 8.03 (1H, s) |
| 1343 | 2.33 (3H, s), 3.24 (3H, s), 3.70 (1H, ddd, J = 15.0, 11.6, 3.9 Hz), 3.85 (1H, dd, J = |
| | 10.3, 3.5 Hz), 3.96 (1H, dd, J = 10.3, 5.6 Hz), 4.08 (1H, td, J = 12.0, 4.3 Hz), 4.24 (1H, |
| | d, J = 12.3 Hz), 4.31 (2H, d, J = 5.3 Hz), 4.66 (1H, dd, J = 14.6, 4.1 Hz), 5.98-6.05 |
| | (1H, m), 6.32 (1H, t, J = 5.4 Hz), 6.49 (2H, s), 6.57 (1H, d, J = 7.7 Hz), 6.94 (1H, s), |
| | 7.13-7.22 (2H, m), 7.33 (1H, d, J = 8.3 Hz), 7.71 (1H, d, J = 6.1 Hz), 7.97 (1H, s) |
| 1344 | 2.33 (3H, s), 3.24 (3H, s), 3.70 (1H, ddd, J = 14.9, 11.6, 3.8 Hz), 3.85 (1H, dd, J = |
| | 10.3, 3.5 Hz), 3.96 (1H, dd, J = 10.3, 5.6 Hz), 4.07 (1H, td, J = 12.0, 4.2 Hz), 4.21- |
| | 4.28 (1H, m), 4.31 (2H, d, J = 5.3 Hz), 4.66 (1H, dd, J = 14.3, 4.1 Hz), 5.99-6.05 (1H, |
| | m), 6.32 (1H, t, J = 5.5 Hz), 6.49 (2H, s), 6.57 (1H, d, J = 7.7 Hz), 6.94 (1H, s), 7.13- |
| | 7.21 (2H, m), 7.33 (1H, d, J = 8.3 Hz), 7.71 (1H, d, J = 6.1 Hz), 7.97 (1H, s) |
| 1345 | 1.47 (3H, d, J = 6.7 Hz), 2.37 (3H, s), 3.60 (1H, ddd, J = 14.4, 9.8, 4.8 Hz), 4.03-4.12 |
| | (1H, m), 4.16-4.28 (2H, m), 4.39 (2H, d, J = 4.7 Hz), 5.48 (1H, q, J = 6.7 Hz), 6.47- |
| | 6.57 (3H, m), 6.77 (1H, d, J = 7.6 Hz), 7.01-7.07 (1H, m), 7.21 (1H, t, J = 8.0 Hz), |
| | 7.34 (1H, d, J = 8.3 Hz), 7.41 (1H, d, J = 2.8 Hz), 7.76 (1H, d, J = 6.0 Hz), 8.28 (1H, d, |
| | J = 2.8 Hz) |
| 1346 | 1.47 (3H, d, J = 6.7 Hz), 2.37 (3H, s), 3.60 (1H, ddd, J = 14.4, 9.9, 4.8 Hz), 4.02-4.12 |
| | (1H, m), 4.16-4.29 (2H, m), 4.39 (2H, d, J = 4.7 Hz), 5.48 (1H, q, J = 6.7 Hz), 6.46- |
| | 6.59 (3H, m), 6.77 (1H, d, J = 7.7 Hz), 7.05 (1H, d, J = 6.1 Hz), 7.21 (1H, t, J = 8.0 Hz), |
| | 7.34 (1H, d, J = 8.3 Hz), 7.41 (1H, d, J = 2.8 Hz), 7.76 (1H, d, J = 6.0 Hz), 8.28 (1H, d, |
| | J = 2.8 Hz) |
| 2177 | 1.11-1.19 (3H, m), 1.44-1.50 (2H, m), 1.64 (2H, d, J = 8.4 Hz), 1.76 (2H, s), 2.11 |
| | (3H, s), 2.53-2.58 (2H, m), 2.68-2.74 (2H, m), 4.31 (2H, d, J = 5.8 Hz), 6.26-6.28 |
| | (2H, m), 6.47 (1H, d, J = 2.4 Hz), 6.53 (1H, d, J = 5.8 Hz), 6.70 (1H, t, J = 5.8 Hz), 6.87 |
| | (1H, dd, J = 9.1, 2.4 Hz), 7.15 (2H, d, J = 8.0 Hz), 7.29 (2H, d, J = 8.0 Hz), 7.53 (1H, d, |
| | J = 5.8 Hz), 7.84 (1H, d, J = 9.1 Hz) |
| 2178 | 1.11-1.20 (3H, m), 1.40-1.49 (2H, m), 1.62-1.68 (2H, m), 1.79-1.89 (2H, m), |
| | 2.16 (3H, s), 2.52-2.56 (2H, m), 2.71-2.79 (2H, m), 4.41 (2H, d, J = 6.0 Hz), 6.45 |
| | (1H, d, J = 7.7 Hz), 6.48 (2H, s), 6.72 (1H, t, J = 6.0 Hz), 7.08-7.13 (3H, m), 7.20 (1H, |
| | d, J = 6.2 Hz), 7.25-7.31 (3H, m), 7.74 (1H, d, J = 6.2 Hz) |
| 2179 | 2.12 (3H, s), 2.14-2.19 (2H, m), 2.19-2.25 (2H, m), 3.41-3.48 (4H, m), 3.67 (2H, |
| | s), 4.35 (2H, d, J = 5.8 Hz), 6.50 (1H, d, J = 2.3 Hz), 6.55-6.66 (3H, m), 6.83-6.95 |
| | (2H, m), 7.15-7.22 (2H, m), 7.29-7.35 (2H, m), 7.51 (1H, d, J = 6.0 Hz), 7.89 (1H, d, |
| | J = 9.0 Hz) |
| 2180 | 2.14 (3H, s), 2.19-2.49 (9H, m), 2.65-2.74 (2H, m), 4.31 (2H, d, J = 5.8 Hz), 6.29 |
| | (2H, s), 6.47 (1H, d, J = 2.3 Hz), 6.53 (1H, d, J = 5.9 Hz), 6.71 (1H, t, J = 5.9 Hz), 6.87 |
| | (1H, dd, J = 9.0, 2.3 Hz), 7.15-7.20 (2H, m), 7.26-7.32 (2H, m), 7.53 (1H, d, J = 5.8 |
| | Hz), 7.80-7.87 (1H, m) |
| 2181 | 2.14 (3H, s), 2.19-2.47 (8H, m), 2.47-2.50 (2H, m), 2.67-2.73 (2H, m), 4.35 (2H, |
| | d, J = 5.9 Hz), 6.29 (2H, s), 6.49 (1H, d, J = 2.4 Hz), 6.56 (1H, d, J = 5.9 Hz), 6.66 (1H, |
| | t, J = 6.0 Hz), 6.88 (1H, dd, J = 9.0, 2.4 Hz), 7.01 (1H, dd, J = 7.8, 1.6 Hz), 7.09 (1H, |
| | dd, J = 11.4, 1.6 Hz), 7.27-7.33 (1H, m), 7.54 (1H, d, J = 5.8 Hz), 7.85 (1H, d, J = 9.1 Hz) |
| 2182 | CDCl₃ 2.30 (3H, s), 2.36-2.84 (12H, m), 4.51 (2H, s), 4.65 (1H, s), 5.17 (2H, s), 6.75 |
| | (1H, d, J = 7.8 Hz), 6.96 (3H, t, J = 6.5 Hz), 7.14 (1H, d, J = 8.3 Hz), 7.31 (2H, dt, J = |
| | 18.6, 8.0 Hz), 7.91 (1H, d, J = 6.2 Hz) |
| 2183 | 2.36-2.41 (4H, m), 2.44-2.49 (2H, m), 2.70 (2H, dd, J = 8.8, 6.6 Hz), 3.55 (4H, t, J = |
| | 4.6 Hz), 4.44 (2H, d, J = 5.8 Hz), 6.45 (1H, d, J = 7.6 Hz), 6.50 (2H, s), 6.65 (1H, t, J = |
| | 6.0 Hz), 6.96 (1H, dd, J = 7.8, 1.6 Hz), 7.08 (1H, dd, J = 11.5, 1.6 Hz), 7.14 (1H, t, J = |
| | 8.0 Hz), 7.17-7.26 (2H, m), 7.32 (1H, d, J = 8.3 Hz), 7.74 (1H, d, J = 6.1 Hz) |
| 2184 | CDCl₃ 2.30 (3H, s), 2.35-2.85 (12H, m), 4.50 (2H, d, J = 5.7 Hz), 4.56-4.64 (1H, m), |
| | 4.93 (2H, s), 6.87 (1H, dd, J = 9.0, 2.4 Hz), 6.92-7.00 (2H, m), 7.04 (1H, d, J = 2.3 |
| | Hz), 7.30 (1H, t, J = 7.7 Hz), 7.57 (1H, d, J = 9.0 Hz), 7.85 (1H, s) |
| 2185 | 1.45-1.53 (2H, m), 1.77-1.87 (2H, m), 1.93-2.01 (2H, m), 2.12 (3H, s), 2.54-2.63 |
| | (2H, m), 3.30-3.39 (1H, m), 4.35 (2H, d, J = 5.9 Hz), 4.45 (2H, s), 6.28 (2H, s), 6.46 |
| | (1H, d, J = 2.3 Hz), 6.52 (1H, d, J = 5.8 Hz), 6.75 (1H, t, J = 6.0 Hz), 6.87 (1H, dd, J = |
| | 9.0, 2.4 Hz), 7.25-7.30 (2H, m), 7.32-7.37 (2H, m), 7.53 (1H, d, J = 5.8 Hz), 7.84 |
| | (1H, d, J = 9.0 Hz) |
| 2186 | 1.43-1.57 (2H, m), 1.79-1.89 (2H, m), 2.02-2.14 (2H, m), 2.17 (3H, s), 2.59-2.66 |
| | (2H, m), 3.37-3.39 (1H, m), 4.44 (2H, s), 4.45 (2H, s), 6.43 (1H, d, J = 7.7 Hz), 6.50 |
| | (2H, s), 6.76 (1H, t, J = 6.0 Hz), 7.07-7.13 (1H, m), 7.21 (1H, d, J = 6.1 Hz), 7.25 (2H, |
| | d, J = 8.0 Hz), 7.30 (1H, d, J = 8.3 Hz), 7.34 (2H, d, J = 8.0 Hz), 7.75 (1H, d, J = 6.1 Hz) |
| 2187 | 1.44-1.52 (2H, m), 1.80-1.87 (2H, m), 1.95-2.02 (2H, m), 2.12 (3H, s), 2.55-2.62 |
| | (2H, m), 3.33-3.37 (1H, m), 4.30 (2H, d, J = 6.0 Hz), 4.45 (2H, s), 5.35 (2H, s), 6.24 |
| | (1H, t, J = 6.0 Hz), 6.50 (1H, s), 6.59 (1H, d, J = 2.3 Hz), 7.09 (1H, dd, J = 8.9, 2.3 Hz), |
| | 7.28 (2H, d, J = 8.0 Hz), 7.32 (1H, d, J = 8.9 Hz), 7.37 (2H, d, J = 8.0 Hz), 8.44 (1H, s) |
| 2188 | 1.42-1.54 (2H, m), 1.77-1.88 (2H, m), 1.94-2.02 (2H, m), 2.12 (3H, s), 2.54-2.61 |
| | (2H, m), 3.33-3.36 (1H, m), 4.28 (2H, d, J = 6.0 Hz), 4.45 (2H, s), 5.84 (2H, s), 6.09 |
| | (1H, t, J = 6.0 Hz), 6.54 (1H, d, J = 2.6 Hz), 6.60 (1H, d, J = 8.8 Hz), 7.01 (1H, dd, J = |
| | 9.0, 2.6 Hz), 7.24 (1H, d, J = 9.0 Hz), 7.27 (2H, d, J = 8.0 Hz), 7.36 (2H, d, J = 8.0 Hz), |
| | 7.58 (1H, d, J = 8.8 Hz) |
| 2189 | 1.43-1.52 (2H, m), 1.80-1.86 (2H, m), 1.96-2.02 (2H, m), 2.12 (3H, s), 2.54-2.61 |
| | (2H, m), 3.34-3.37 (1H, m), 4.33 (2H, d, J = 5.9 Hz), 4.45 (2H, s), 5.48 (2H, s), 6.17- |
| | 6.23 (2H, m), 6.67 (1H, dd, J = 8.9, 2.2 Hz), 6.75 (1H, t, J = 5.9 Hz), 7.28 (2H, d, J = |
| | 8.1 Hz), 7.35 (2H, d, J = 8.1 Hz), 7.44 (1H, d, J = 8.9 Hz), 8.37 (1H, s) |
| 2190 | 1.42-1.53 (2H, m), 1.78-1.86 (2H, m), 1.95-2.05 (2H, m), 2.12 (3H, s), 2.53-2.62 |
| | (2H, m), 3.10 (3H, s), 3.29-3.33 (1H, m), 4.44 (2H, s), 4.70 (2H, s), 6.40 (2H, s), 6.63 |
| | (1H, d, J = 5.9 Hz), 6.69 (1H, d, J = 2.6 Hz), 7.04 (1H, dd, J = 9.3, 2.7 Hz), 7.16-7.21 |
| | (2H, m), 7.23-7.28 (2H, m), 7.57 (1H, d, J = 5.9 Hz), 7.94 (1H, d, J = 9.2 Hz) |
| 2191 | 1.43-1.55 (2H, m), 1.79-1.87 (2H, m), 1.95-2.03 (2H, m), 2.12 (3H, s), 2.54-2.61 |
| | (2H, m), 3.33-3.38 (1H, m), 4.38 (2H, d, J = 5.8 Hz), 4.47 (2H, s), 6.30 (2H, s), 6.49 |
| | (1H, d, J = 2.3 Hz), 6.55 (1H, d, J = 5.9 Hz), 6.70 (1H, t, J = 6.0 Hz), 6.88 (1H, dd, J = |
| | 9.0, 2.4 Hz), 7.07-7.12 (1H, m), 7.12-7.17 (1H, m), 7.37 (1H, app t, J = 7.8 Hz), |
| | 7.54 (1H, d, J = 5.8 Hz), 7.86 (1H, d, J = 9.0 Hz) |
| 2192 | 1.44-1.53 (2H, m), 1.79-1.86 (2H, m), 1.95-2.02 (2H, m), 2.12 (3H, s), 2.54-2.61 |
| | (2H, m), 3.32-3.38 (1H, m), 4.41 (2H, d, J = 5.9 Hz), 4.47 (2H, s), 6.30 (2H, s), 6.41 |
| | (1H, d, J = 2.3 Hz), 6.54 (1H, d, J = 5.8 Hz), 6.78 (1H, t, J = 6.0 Hz), 6.89 (1H, dd, J = |
| | 9.0, 2.4 Hz), 7.23 (1H, dd, J = 8.0, 1.7 Hz), 7.38 (1H, d, J = 7.9 Hz), 7.42 (1H, d, J = 1.6 |
| | Hz), 7.54 (1H, d, J = 5.9 Hz), 7.87 (1H, d, J = 9.0 Hz) |
| 2193 | 1.40-1.57 (2H, m), 1.77-1.87 (2H, m), 1.91-2.05 (2H, m), 2.12 (3H, s), 2.54-2.62 |
| | (2H, m), 3.34-3.37 (1H, m), 4.39 (2H, d, J = 5.9 Hz), 4.46 (2H, s), 6.61 (1H, d, J = 2.2 |
| | Hz), 7.11 (1H, t, J = 5.9 Hz), 7.14 (1H, dd, J = 8.9, 2.3 Hz), 7.26-7.32 (2H, m), 7.34 |
| | (1H, d, J = 5.8 Hz), 7.35-7.40 (2H, m), 7.75 (1H, d, J = 8.9 Hz), 8.15 (1H, d, J = 5.8 |
| | Hz), 8.85 (1H, s) |
| 2194 | 1.66-1.77 (1H, m), 1.80-1.89 (1H, m), 2.16-2.24 (4H, m), 2.42-2.51 (2H, m), |
| | 2.68-2.80 (1H, m), 3.62-3.71 (1H, m), 4.36 (2H, d, J = 5.6 Hz), 4.61 (2H, s), 6.32 |
| | (2H, d, J = 6.7 Hz), 6.47 (1H, d, J = 2.3 Hz), 6.53 (1H, d, J = 5.9 Hz), 6.78 (1H, t, J = 6.0 |
| | Hz), 6.88 (1H, dd, J = 9.0, 2.3 Hz), 7.27-7.32 (2H, m), 7.35-7.39 (2H, m), 7.53 (1H, |
| | d, J = 5.8 Hz), 7.85 (1H, d, J = 9.0 Hz), 2 × C-H signals obscured by DMSO |
| 2195 | 0.20-0.30 (2H, m), 0.33-0.41 (2H, m), 1.32-1.48 (2H, m), 1.51-1.59 (1H, m), |
| | 1.73-1.85 (2H, m), 2.15-2.31 (2H, m), 2.71-2.84 (2H, m), 3.33-3.40 (1H, m), |
| | 4.29-4.39 (2H, m), 4.42-4.49 (2H, m), 6.22-6.30 (2H, m), 6.42-6.49 (1H, m), |
| | 6.49-6.55 (1H, m), 6.69-6.78 (1H, m), 6.83-6.91 (1H, m), 7.22-7.32 (2H, m), |
| | 7.32-7.39 (2H, m), 7.50-7.55 (1H, m), 7.80-7.87 (1H, m) |
| 2196 | 1.43-1.56 (2H, m), 1.76-1.87 (2H, m), 2.22-2.32 (2H, m), 2.63-2.72 (2H, m), |
| | 2.72-2.81 (2H, m), 3.35-3.41 (1H, m), 4.44 (2H, d, J = 5.7 Hz), 4.46 (2H, s), 6.10 |
| | (1H, tt, J = 55.8, 4.3 Hz), 6.73 (1H, d, J = 2.3 Hz), 6.83 (1H, d, J = 7.0 Hz), 7.07 (1H, |
| | dd, J = 9.1, 2.3 Hz), 7.29-7.32 (2H, m), 7.33-7.37 (2H, m), 7.41 (1H, d, J = 7.0 Hz), |
| | 7.66 (1H, t, J = 6.0 Hz), 8.15 (1H, d, J = 9.2 Hz), 8.23 (2H, s), 12.13 (1H, s) |
| 2197 | 1.43-1.55 (2H, m), 1.78-1.87 (2H, m), 1.94-2.02 (2H, m), 2.12 (3H, s), 2.55-2.60 |
| | (2H, m), 3.32-3.38 (1H, m), 4.46 (2H, s), 4.48 (2H, d, J = 5.8 Hz), 6.44 (1H, d, J = 7.7 |
| | Hz), 6.51 (2H, s), 6.69 (1H, t, J = 5.9 Hz), 7.04-7.07 (1H, m), 7.12-7.16 (2H, m), |
| | 7.20 (1H, d, J = 6.1 Hz), 7.27-7.31 (1H, m), 7.33 (1H, d, J = 8.3 Hz), 7.75 (1H, d, J = |
| | 6.1 Hz) |
| 2198 | 1.21-1.27 (1H, m), 1.45-1.53 (2H, m), 1.78-1.84 (1H, m), 2.01-2.08 (1H, m), |
| | 2.15 (3H, s), 2.42-2.47 (1H, m), 2.62 (1H, s), 3.02-3.07 (1H, m), 3.47-3.53 (1H, |
| | m), 4.38 (2H, d, J = 5.8 Hz), 4.39-4.48 (2H, m), 6.31 (2H, s), 6.48 (1H, d, J = 2.3 Hz), |
| | 6.55 (1H, d, J = 5.8 Hz), 6.71 (1H, t, J = 6.0 Hz), 6.88 (1H, dd, J = 9.0, 2.4 Hz), 7.09 |
| | (1H, dd, J = 7.8, 1.6 Hz), 7.13 (1H, dd, J = 11.1, 1.6 Hz), 7.34-7.39 (1H, m), 7.54 (1H, |
| | d, J = 5.8 Hz), 7.86 (1H, d, J = 9.0 Hz) |
| 2199 | 1.23-1.31 (1H, m), 1.47-1.54 (2H, m), 1.81-1.89 (1H, m), 2.02-2.09 (1H, m), |
| | 2.17 (3H, s), 2.43-2.46 (1H, m), 2.60-2.69 (1H, m), 3.04-3.11 (1H, m), 3.49-3.54 |
| | (1H, m), 4.38 (2H, d, J = 5.7 Hz), 4.39-4.48 (2H, m), 6.32 (2H, s), 6.48 (1H, d, J = 2.4 |
| | Hz), 6.55 (1H, d, J = 5.9 Hz), 6.72 (1H, t, J = 6.0 Hz), 6.88 (1H, dd, J = 9.1, 2.4 Hz), |
| | 7.09 (1H, dd, J = 7.9, 1.6 Hz), 7.14 (1H, dd, J = 11.1, 1.6 Hz), 7.34-7.39 (1H, m), 7.54 |
| | (1H, d, J = 5.8 Hz), 7.86 (1H, d, J = 9.1 Hz) |
| 2200 | 1.34-1.42 (1H, m), 1.44-1.52 (1H, m), 1.75-1.82 (1H, m), 1.82-1.90 (1H, m), |
| | 1.98 (3H, s), 3.08 (1H, ddd, J = 12.8, 9.0, 3.5 Hz), 3.19 (1H, ddd, J = 13.0, 8.9, 3.4 |
| | Hz), 3.57-3.65 (2H, m), 3.77-3.84 (1H, m), 4.39 (2H, d, J = 5.9 Hz), 4.52 (2H, s), |
| | 6.30 (2H, s), 6.49 (1H, d, J = 2.3 Hz), 6.55 (1H, d, J = 5.9 Hz), 6.71 (1H, t, J = 6.0 Hz), |
| | 6.88 (1H, dd, J = 9.0, 2.4 Hz), 7.12 (1H, dd, J = 7.8, 1.6 Hz), 7.17 (1H, dd, J = 11.1, 1.6 |
| | Hz), 7.36-7.40 (1H, m), 7.54 (1H, d, J = 5.8 Hz), 7.86 (1H, d, J = 9.0 Hz) |
| 2201 | 0.97 (3H, t, J = 7.2 Hz), 1.40-1.52 (2H, m), 1.80-1.88 (2H, m), 1.94-2.05 (2H, m), |
| | 2.27 (2H, q, J = 7.2 Hz), 2.60-2.73 (2H, m), 3.34-3.39 (1H, m), 4.46 (2H, s), 4.47 |
| | (2H, d, J = 6.0 Hz), 6.44 (1H, d, J = 7.7 Hz), 6.51 (2H, s), 6.68 (1H, t, J = 6.0 Hz), 7.06 |
| | (1H, dd, J = 7.9, 1.5 Hz), 7.11-7.17 (2H, m), 7.20 (1H, d, J = 6.1 Hz), 7.26-7.31 (1H, |
| | m), 7.33 (1H, d, J = 8.3 Hz), 7.75 (1H, d, J = 6.1 Hz) |
| 2202 | 0.97 (3H, t, J = 7.2 Hz), 1.41-1.54 (2H, m), 1.79-1.88 (2H, m), 1.93-2.06 (2H, m), |
| | 2.27 (2H, q, J = 7.2 Hz), 2.60-2.72 (2H, m), 3.33-3.39 (1H, m), 4.38 (2H, d, J = 5.9 |
| | Hz), 4.48 (2H, s), 6.30 (2H, s), 6.48 (1H, d, J = 2.3 Hz), 6.55 (1H, d, J = 5.8 Hz), 6.70 |
| | (1H, t, J = 6.0 Hz), 6.88 (1H, dd, J = 9.0, 2.3 Hz), 7.10 (1H, dd, J = 7.9, 1.6 Hz), 7.15 |
| | (1H, dd, J = 11.0, 1.5 Hz), 7.33-7.41 (1H, m), 7.54 (1H, d, J = 5.8 Hz), 7.86 (1H, d, J = |
| | 9.1 Hz) |
| 2203 | 1.53-1.65 (2H, m), 1.85-1.93 (2H, m), 2.24-2.39 (5H, m), 2.76 (3H, s), 2.77-2.83 |
| | (2H, m), 3.42-3.50 (1H, m), 4.29 (2H, s), 4.49 (2H, s), 6.73 (2H, s), 7.06 (1H, d, J = |
| | 6.0 Hz), 7.28-7.32 (2H, m), 7.34-7.38 (1H, m), 7.40 (1H, s), 7.57 (1H, d, J = 7.8 Hz), |
| | 7.76 (1H, d, J = 6.0 Hz), 7.85 (1H, d, J = 8.2 Hz) |
| 2204 | 1.41-1.52 (2H, m), 1.77-1.86 (2H, m), 1.93-2.02 (2H, m), 2.11 (3H, s), 2.53-2.61 |
| | (2H, m), 3.16 (3H, s), 3.30-3.36 (1H, m), 4.44 (2H, s), 4.76 (2H, s), 6.88 (1H, d, J = |
| | 2.5 Hz), 7.18-7.22 (2H, m), 7.25-7.28 (2H, m), 7.32 (1H, dd, J = 9.2, 2.6 Hz), 7.45 |
| | (1H, d, J = 5.9 Hz), 7.85 (1H, d, J = 9.2 Hz), 8.21 (1H, d, J = 5.8 Hz), 8.92 (1H, s) |
| 2205 | 1.41-1.51 (1H, m), 1.69-1.77 (2H, m), 2.16-2.22 (1H, m), 2.23-2.30 (1H, m), |
| | 2.38 (3H, s), 2.58-2.65 (1H, m), 3.40-3.47 (1H, m), 4.20 (1H, d, J = 6.4 Hz), 4.24 |
| | (1H, d, J = 6.6 Hz), 4.39 (2H, d, J = 5.5 Hz), 4.51 (2H, s), 4.57 (1H, d, J = 6.4 Hz), 4.62 |
| | (1H, d, J = 6.6 Hz), 6.47 (2H, s), 6.51 (1H, d, J = 2.3 Hz), 6.58 (1H, d, J = 6.0 Hz), 6.78 |
| | (1H, t, J = 6.1 Hz), 6.90 (1H, dd, J = 9.1, 2.4 Hz), 7.11 (1H, d, J = 7.8 Hz), 7.15 (1H, d, |
| | J = 11.1 Hz), 7.38 (1H, t, J = 7.9 Hz), 7.53 (1H, d, J = 6.0 Hz), 7.89 (1H, d, J = 9.0 Hz) |
| 2207 | 3.61 (3H, s), 4.45 (2H, s), 4.48 (2H, d, J = 5.6 Hz), 4.53 (2H, s), 6.43 (1H, d, J = 7.7 |
| | Hz), 6.50 (2H, s), 6.69 (1H, t, J = 5.7 Hz), 6.80 (1H, d, J = 0.7 Hz), 7.06 (1H, d, J = 7.9 |
| | Hz), 7.11-7.20 (4H, m), 7.28-7.33 (2H, d, m), 7.75 (1H, d, J = 6.0 Hz) |
| 2208 | CDCl₃ 2.99 (2H, t, J = 6.8 Hz), 3.59 (3H, s), 3.87 (2H, t, J = 6.9 Hz), 4.50 (2H, s), 4.53 |
| | (2H, d, J = 5.2 Hz), 4.68 (1H, s), 5.14 (2H, s), 6.73 (1H, d, J = 7.7 Hz), 6.79 (1H, d, J = |
| | 1.4 Hz), 6.93 (1H, d, J = 1.4 Hz), 6.96-7.06 (3H, m), 7.15 (1H, d, J = 8.3 Hz), 7.33 |
| | (2H, t, J = 7.9 Hz), 7.93 (1H, d, J = 6.1 Hz) |
| 2209 | CDCl₃ 1.93-2.10 (2H, m), 2.35 (1H, dt, J = 17.5, 6.0 Hz), 2.53-2.64 (1H, m), 2.92 |
| | (3H, s), 3.35 (1H, ddd, J = 12.7, 4.6, 1.3 Hz), 3.46 (1H, dd, J = 12.7, 4.2 Hz), 3.80- |
| | 3.89 (1H, m), 4.48-4.55 (2H, m), 4.56-4.63 (2H, m), 4.70 (1H, s), 5.20 (2H, s), |
| | 6.70-6.76 (1H, m), 6.98 (1H, dd, J = 6.3, 1.0 Hz), 7.03-7.08 (1H, m), 7.10 (1H, dd, |
| | J = 10.7, 1.6 Hz), 7.15 (1H, dt, J = 8.4, 1.0 Hz), 7.32 (1H, d, J = 8.0 Hz), 7.38 (1H, d, J = |
| | 7.8 Hz), 7.92 (1H, d, J = 6.2 Hz) |
| 2210 | 1.40-1.55 (2H, m), 1.76-1.88 (2H, m), 1.96-2.06 (2H, m), 2.14 (3H, s), 2.55-2.63 |
| | (2H, m), 3.25-3.42 (1H, m), 4.51 (2H, s), 6.69 (2H, s), 6.72 (1H, d, J = 5.9 Hz), 7.16 |
| | (1H, dd, J = 8.9, 2.2 Hz), 7.27 (1H, d, J = 2.2 Hz), 7.43-7.50 (2H, m), 7.67 (1H, d, J = |
| | 5.8 Hz), 7.74-7.82 (2H, m), 8.01 (1H, d, J = 9.1 Hz), 10.60 (1H, br.s) |
| 2211 | 3.56 (3H, s), 3.64 (2H, s), 4.25 (2H, d, J = 5.9 Hz), 4.46 (2H, d, J = 5.8 Hz), 6.42 (1H, d, |
| | J = 7.7 Hz), 6.50 (2H, s), 6.68 (1H, t, J = 6.0 Hz), 6.74 (1H, d, J = 1.2 Hz), 6.98-7.01 |
| | (1H, m), 7.03 (1H, d, J = 1.2 Hz), 7.06-7.11 (1H, m), 7.13 (1H, t, J = 8.0 Hz), 7.19 |
| | (1H, d, J = 6.0 Hz), 7.26 (1H, t, J = 7.9 Hz), 7.32 (1H, d, J = 8.3 Hz), 7.75 (1H, d, J = 6.0 |
| | Hz), 8.68 (1H, t, J = 5.9 Hz) |
| 2212 | 0.93 (6H, dd, J = 14.9, 6.1 Hz), 1.50 (2H, q, J = 9.0 Hz), 2.40 (1H, d, J = 9.4 Hz), 2.52- |
| | 2.65 (6H, m), 2.65-2.74 (2H, m), 3.20 (2H, s), 4.44 (2H, d, J = 5.8 Hz), 6.45 (1H, d, |
| | J = 7.7 Hz), 6.49 (2H, s), 6.65 (1H, t, J = 6.0 Hz), 6.95 (1H, dd, J = 7.8, 1.6 Hz), 7.07 |
| | (1H, dd, J = 11.5, 1.6 Hz), 7.13 (1H, t, J = 8.0 Hz), 7.17-7.24 (2H, m), 7.32 (1H, d, J = |
| | 8.3 Hz), 7.74 (1H, d, J = 6.1 Hz) |
| 2213 | 1.06 (6H, dd, J = 21.2, 6.2 Hz), 1.72 (2H, q, J = 10.2 Hz), 2.62-2.98 (9H, m), 3.44 |
| | (1H, s), 3.80 (1H, s), 4.45 (2H, d, J = 5.7 Hz), 6.45 (1H, d, J = 7.7 Hz), 6.61 (2H, s), |
| | 6.68 (1H, t, J = 6.0 Hz), 6.97 (1H, dd, J = 7.9, 1.6 Hz), 7.06-7.27 (4H, m), 7.33 (1H, d, |
| | J = 8.4 Hz), 7.74 (1H, d, J = 6.1 Hz), 8.25 (2H, s) |
| 2214 | 0.93 (6H, dd, J = 16.8, 6.1 Hz), 1.45-1.58 (2H, m), 2.42 (1H, d, J = 9.4 Hz), 2.52- |
| | 2.74 (8H, m), 3.22 (1H, s), 3.42 (1H, s), 4.38 (2H, d, J = 5.8 Hz), 6.55 (2H, s), 6.70 |
| | (1H, d, J = 2.4 Hz), 6.94 (1H, dd, J = 9.0, 2.4 Hz), 7.01 (1H, dd, J = 7.8, 1.6 Hz), 7.04- |
| | 7.14 (2H, m), 7.30 (1H, t, J = 8.0 Hz), 7.64 (1H, s), 7.92 (1H, d, J = 9.1 Hz) |
| 2215 | 2.36-2.43 (4H, m), 2.72 (2H, t, J = 7.7 Hz), 3.55 (6H, t, J = 4.6 Hz, in water), 4.38 |
| | (2H, d, J = 5.7 Hz), 6.55 (2H, s), 6.71 (1H, d, J = 2.3 Hz), 6.95 (1H, dd, J = 9.1, 2.4 Hz), |
| | 7.02 (1H, dd, J = 7.8, 1.6 Hz), 7.05-7.15 (2H, m), 7.31 (1H, t, J = 8.0 Hz), 7.65 (1H, |
| | s), 7.92 (1H, d, J = 9.1 Hz), 8.25 (2H, s) |
| 2216 | 0.93 (6H, d, J = 6.6 Hz), 1.38-1.47 (2H, m), 1.81-1.87 (2H, m), 2.11-2.17 (2H, m), |
| | 2.61-2.67 (3H, m), 3.27-3.30 (1H, m), 4.37 (2H, d, J = 5.8 Hz), 4.47 (2H, s), 6.30 |
| | (2H, s), 6.47 (1H, d, J = 2.2 Hz), 6.54 (1H, d, J = 5.8 Hz), 6.70 (1H, t, J = 5.9 Hz), 6.87 |
| | (1H, dd, J = 9.0, 2.3 Hz), 7.08-7.16 (2H, m), 7.36 (1H, t, J = 7.8 Hz), 7.54 (1H, d, J = |
| | 5.8 Hz), 7.85 (1H, d, J = 9.0 Hz) |
| 2252 | 2.10 (1H, dd, J = 13.1, 4.0 Hz), 2.16 (3H, s), 2.43 (1H, dd, J = 13.1, 4.3 Hz), 2.90 (1H, |
| | dd, J = 11.1, 3.5 Hz), 3.00-3.05 (2H, m), 3.10 (1H, d, J = 7.1 Hz), 3.34 (1H, d, J = 7.3 |
| | Hz), 3.44 (1H, d, J = 7.1 Hz), 4.19-4.25 (1H, m), 4.38 (2H, d, J = 5.5 Hz), 4.49 (2H, d, |
| | J = 2.2 Hz), 6.27-6.32 (2H, m), 6.49 (1H, d, J = 2.3 Hz), 6.55 (1H, d, J = 5.9 Hz), 6.70 |
| | (1H, t, J = 5.8 Hz), 6.88 (1H, dd, J = 9.3, 2.3 Hz), 7.10 (1H, d, J = 7.7 Hz), 7.15 (1H, d, |
| | J = 11.0 Hz), 7.38 (1H, t, J = 7.9 Hz), 7.54 (1H, d, J = 5.8 Hz), 7.86 (1H, d, J = 9.0 Hz) |
| 2253 | 2.64 (1H, dd, J = 14.0, 3.2 Hz), 3.13 (1H, dd, J = 14.0, 7.1 Hz), 4.14 (2H, s), 4.17 (2H, |
| | d, J = 2.4 Hz), 4.34 (2H, d, J = 5.8 Hz), 4.85-4.96 (1H, m), 5.67 (1H, d, J = 5.7 Hz), |
| | 6.47 (1H, d, J = 8.4 Hz), 6.49 (2H, s), 6.56 (1H, d, J = 7.7 Hz), 6.63 (1H, t, J = 6.0 Hz), |
| | 7.00 (1H, d, J = 1.3 Hz), 7.11-7.18 (2H, m), 7.31 (1H, d, J = 8.3 Hz), 7.35 (1H, d, J = |
| | 1.3 Hz), 7.59 (1H, dd, J = 8.5, 2.3 Hz), 7.73 (1H, d, J = 6.1 Hz), 8.17 (1H, d, J = 2.3 Hz) |
| 2254 | 1.75 (2H, tdd, J = 13.2, 5.1, 1.9 Hz), 1.80-1.89 (2H, m), 2.37 (3H, s), 2.68 (2H, d, J = |
| | 4.2 Hz), 3.54-3.61 (2H, m), 3.65 (2H, dt, J = 10.9, 2.2 Hz), 4.31 (1H, tt, J = 10.5, 6.4 |
| | Hz), 4.48 (4H, d, J = 4.3 Hz), 6.45 (1H, d, J = 7.7 Hz), 6.60 (2H, s), 6.71 (1H, t, J = 6.0 |
| | Hz), 7.06 (1H, dd, J = 7.8, 1.6 Hz), 7.11-7.18 (2H, m), 7.19-7.24 (1H, m), 7.29 (1H, |
| | t, J = 7.9 Hz), 7.34 (1H, d, J = 8.3 Hz), 7.75 (1H, d, J = 6.1 Hz) |
| 2255 | 1.39-1.58 (2H, m), 1.77-1.89 (2H, m), 1.92-2.05 (2H, m), 2.12 (3H, s), 2.53-2.62 |
| | (2H, m), 3.33-3.37 (1H, m), 4.39 (2H, d, J = 5.9 Hz), 4.46 (2H, s), 6.81 (1H, d, J = 2.3 |
| | Hz), 6.84 (1H, t, J = 5.9 Hz), 7.25-7.33 (3H, m), 7.36-7.43 (2H, m), 7.52 (1H, d, J = |
| | 5.5 Hz), 7.66 (1H, d, J = 8.9 Hz), 8.12 (1H, d, J = 5.5 Hz), 8.88 (1H, s) |
| 2256 | 3.78 (2H, t, J = 5.5 Hz), 4.24 (2H, t, J = 5.5 Hz), 4.39 (2H, d, J = 5.8 Hz), 4.67 (2H, s), |
| | 6.45-6.51 (3H, m), 6.59 (1H, t, J = 6.0 Hz), 6.86 (1H, dd, J = 8.7, 2.5 Hz), 7.00 (1H, |
| | dd, J = 13.5, 2.5 Hz), 7.14 (1H, t, J = 8.0 Hz), 7.18 (1H, d, J = 6.1 Hz), 7.22 (1H, t, J = |
| | 8.8 Hz), 7.31 (1H, d, J = 8.4 Hz), 7.74 (1H, d, J = 6.1 Hz) |
| 2257 | 2.35 (3H, s), 3.73 (2H, t, J = 5.5 Hz), 4.24 (2H, t, J = 5.5 Hz), 4.32 (2H, d, J = 5.4 Hz), |
| | 4.61 (2H, s), 6.40 (1H, d, J = 7.7 Hz), 6.47-6.53 (3H, m), 6.84 (1H, dd, J = 8.4, 2.7 |
| | Hz), 6.98 (1H, d, J = 2.7 Hz), 7.08-7.16 (2H, m), 7.22 (1H, d, J = 6.1 Hz), 7.30 (1H, d, |
| | J = 8.3 Hz), 7.73 (1H, d, J = 6.1 Hz). |
| 3253 | 1.10-1.22 (2H, m), 1.37-1.44 (2H, m), 1.62-1.78 (3H, m), 2.11 (3H, s), 2.66-2.73 |
| | (2H, m), 3.93 (2H, d, J = 7.2 Hz), 4.15 (2H, d, J = 5.4 Hz), 6.28 (2H, s), 6.36 (1H, t, J = |
| | 5.5 Hz), 6.54 (1H, d, J = 2.3 Hz), 6.59 (1H, d, J = 5.8 Hz), 6.86 (1H, dd, J = 9.0, 2.3 Hz), |
| | 7.42 (1H, s), 7.56 (1H, d, J = 5.8 Hz), 7.66 (1H, s), 7.83 (1H, d, J = 9.0 Hz) |
| 3254 | 1.04-1.18 (2H, m), 1.54-1.74 (7H, m), 2.09 (3H, s), 2.62-2.70 (2H, m), 4.07 (2H, t, |
| | J = 7.2 Hz), 4.15 (2H, d, J = 5.3 Hz), 6.28 (2H, s), 6.37 (1H, t, J = 5.4 Hz), 6.54 (1H, d, |
| | J = 2.3 Hz), 6.59 (1H, d, J = 5.9 Hz), 6.86 (1H, dd, J = 9.0, 2.3 Hz), 7.42 (1H, s), 7.55 |
| | (1H, d, J = 5.8 Hz), 7.69 (1H, s), 7.83 (1H, d, J = 9.1 Hz) |
| 3255 | 1.04-1.21 (3H, m), 1.57-1.68 (4H, m), 1.81-1.94 (2H, m), 2.20 (3H, s), 2.74-2.81 |
| | (2H, m), 4.05 (2H, t, J = 7.2 Hz), 4.26 (2H, d, J = 5.6 Hz), 6.32 (1H, t, J = 5.8 Hz), 6.49 |
| | (2H, s), 6.63 (1H, d, J = 7.7 Hz), 7.12 (1H, d, J = 6.1 Hz), 7.18 (1H, t, J = 8.0 Hz), 7.32 |
| | (1H, d, J = 8.3 Hz), 7.40 (1H, d, J = 0.7 Hz), 7.64 (1H, s), 7.71 (1H, d, J = 6.1 Hz) |
| 4259 | 1.57-1.68 (2H, m), 1.89-1.96 (2H, m), 2.05-2.18 (5H, m), 2.56-2.65 (2H, m), |
| | 4.36 (2H, d, J = 6.3 Hz), 4.90-4.96 (1H, m), 6.31 (2H, s), 6.43 (1H, d, J = 2.4 Hz), |
| | 6.53 (1H, d, J = 5.8 Hz), 6.71 (1H, s), 6.82 (1H, t, J = 6.3 Hz), 6.88 (1H, dd, J = 9.0, 2.4 |
| | Hz), 6.95 (1H, dd, J = 5.2, 1.4 Hz), 7.54 (1H, d, J = 5.8 Hz), 7.87 (1H, d, J = 9.0 Hz), |
| | 8.06 (1H, d, J = 5.2 Hz) |
| 4260 | 1.38-1.54 (2H, m), 1.84-2.05 (3H, m), 2.75 (3H, s), 2.87-2.99 (2H, m), 3.38-3.46 |
| | (2H, m), 4.11 (2H, s), 4.50 (2H, d, J = 5.9 Hz), 6.71 (1H, d, J = 8.0 Hz), 6.74 (1H, s), |
| | 6.98-7.01 (1H, m), 7.30 (1H, s), 7.41 (1H, t, J = 8.1 Hz), 7.52 (1H, d, J = 7.0 Hz), 7.64 |
| | (1H, d, J = 8.3 Hz), 7.69 (1H, d, J = 7.2 Hz), 8.07 (1H, d, J = 5.3 Hz), 8.50 (2H, s), 9.20 |
| | (1H, s), 12.84 (1H, s) |
| 4261 | 1.17-1.30 (2H, m), 1.60-1.70 (3H, m), 1.77-1.86 (2H, m), 2.13 (3H, s), 2.70-2.78 |
| | (2H, m), 4.06 (2H, d, J = 6.1 Hz), 4.37 (2H, d, J = 6.2 Hz), 6.31 (2H, s), 6.42 (1H, d, J = |
| | 2.3 Hz), 6.53 (1H, d, J = 5.9 Hz), 6.75 (1H, s), 6.83 (1H, t, J = 6.2 Hz), 6.85-6.90 (1H, |
| | m), 6.94-6.99 (1H, m), 7.54 (1H, d, J = 5.8 Hz), 7.87 (1H, d, J = 9.1 Hz), 8.06 (1H, d, |
| | J = 5.3 Hz) |
| 4262 | 1.19-1.30 (2H, m), 1.60-1.69 (3H, m), 1.76-1.85 (2H, m), 2.13 (3H, s), 2.71-2.76 |
| | (2H, m), 4.06 (2H, d, J = 6.1 Hz), 4.42 (2H, d, J = 6.2 Hz), 6.58 (1H, d, J = 2.2 Hz), 6.76 |
| | (1H, s), 6.98 (1H, dd, J = 5.3, 1.4 Hz), 7.12-7.21 (2H, m), 7.35 (1H, d, J = 5.8 Hz), |
| | 7.78 (1H, d, J = 8.9 Hz), 8.07 (1H, d, J = 5.3 Hz), 8.16 (1H, d, J = 5.8 Hz), 8.87 (1H, s) |
| 4263 | 1.19-1.30 (2H, m), 1.61-1.69 (3H, m), 1.77-1.84 (2H, m), 2.13 (3H, s), 2.71-2.76 |
| | (2H, m), 4.06 (2H, d, J = 6.1 Hz), 4.38 (2H, d, J = 6.1 Hz), 6.38 (1H, d, J = 2.3 Hz), 6.74 |
| | (1H, s), 6.88 (1H, dd, J = 9.0, 2.4 Hz), 6.96 (1H, dd, J = 5.3, 1.4 Hz), 7.08 (1H, t, J = |
| | 6.2 Hz), 7.22 (2H, s), 7.88 (1H, d, J = 9.0 Hz), 8.07 (1H, d, J = 5.3 Hz), 8.11 (1H, s) |
| 4264 | 1.20-1.29 (2H, m), 1.61-1.69 (3H, m), 1.77-1.84 (2H, m), 2.13 (3H, s), 2.71-2.76 |
| | (2H, m), 4.06 (2H, d, J = 6.1 Hz), 4.34 (2H, d, J = 6.2 Hz), 6.27 (1H, t, J = 6.4 Hz), 6.79 |
| | (1H, s), 6.83 (1H, d, J = 2.6 Hz), 6.99 (1H, dd, J = 5.3, 1.4 Hz), 7.43 (1H, d, J = 1.9 Hz), |
| | 7.88 (1H, d, J = 2.6 Hz), 8.06 (1H, d, J = 5.2 Hz), 11.48 (1H, s) |
| 4265 | 1.20-1.32 (2H, m), 1.63-1.71 (3H, m), 1.78-1.85 (2H, m), 2.13 (3H, s), 2.71-2.77 |
| | (2H, m), 4.10 (2H, d, J = 6.0 Hz), 4.49 (2H, d, J = 6.2 Hz), 6.34 (2H, s), 6.46 (1H, s), |
| | 6.54 (1H, d, J = 5.9 Hz), 6.86-6.92 (2H, m), 7.05 (1H, s), 7.48 (1H, s), 7.53-7.56 |
| | (1H, m), 7.89 (1H, d, J = 9.0 Hz) |
| 4266 | 1.67-1.79 (1H, m), 2.10-2.18 (1H, m), 2.35-2.45 (1H, m), 2.52-2.59 (1H, m), |
| | 2.95 (1H, dd, J = 16.6, 5.1 Hz), 3.94 (1H, app td, J = 12.0, 4.7 Hz), 4.09-4.18 (1H, |
| | m), 4.25 (2H, d, J = 6.6 Hz), 4.39 (2H, d, J = 6.2 Hz), 6.42 (2H, s), 6.45 (1H, d, J = 2.3 |
| | Hz), 6.55 (1H, d, J = 5.9 Hz), 6.81 (1H, s), 6.87-6.92 (2H, m), 7.00 (1H, dd, J = 5.3, |
| | 1.3 Hz), 7.53 (1H, d, J = 5.9 Hz), 7.66 (1H, d, J = 1.5 Hz), 7.89 (1H, d, J = 9.1 Hz), 8.09 |
| | (1H, d, J = 5.3 Hz) |
| 4267 | 1.65-1.75 (1H, m), 2.07-2.15 (1H, m), 2.29-2.41 (1H, m), 2.43-2.47 (1H, m), |
| | 2.86-2.94 (1H, m), 3.82-3.91 (1H, m), 4.02-4.10 (1H, m), 4.24 (2H, d, J = 6.6 Hz), |
| | 4.39 (2H, d, J = 6.1 Hz), 6.31 (2H, s), 6.44 (1H, d, J = 2.4 Hz), 6.53 (1H, d, J = 5.8 Hz), |
| | 6.79 (1H, d, J = 1.3 Hz), 6.81 (1H, s), 6.85 (1H, t, J = 6.2 Hz), 6.88 (1H, dd, J = 9.0, 2.4 |
| | Hz), 6.94-7.04 (2H, m), 7.54 (1H, d, J = 5.8 Hz), 7.87 (1H, d, J = 9.1 Hz), 8.08 (1H, d, |
| | J = 5.3 Hz) |
| 4268 | 1.49-1.63 (2H, m), 1.85-1.92 (3H, m), 1.93-2.03 (1H, m), 2.69 (3H, s), 2.80-3.02 |
| | (3H, m), 4.13 (2H, d, J = 6.4 Hz), 4.43 (2H, d, J = 6.1 Hz), 6.66 (1H, d, J = 2.3 Hz), 6.75- |
| | 6.82 (3H, m), 6.98 (1H, dd, J = 9.1, 2.4 Hz), 7.01 (1H, dd, J = 5.3, 1.4 Hz), 7.35 (1H, |
| | t, J = 6.2 Hz), 7.67 (1H, s), 7.99 (1H, d, J = 9.1 Hz), 8.09 (1H, d, J = 5.2 Hz), 10.20 (1H, s) |
| 4269 | 1.36-1.41 (2H, m), 1.48-1.54 (1H, m), 2.15-2.21 (5H, m), 2.88 (2H, d, J = 8.7 Hz), |
| | 4.02 (2H, d, J = 7.4 Hz), 4.37 (2H, d, J = 6.2 Hz), 6.31 (2H, s), 6.42 (1H, d, J = 2.3 Hz), |
| | 6.53 (1H, d, J = 5.9 Hz), 6.75 (1H, s), 6.83 (1H, t, J = 6.2 Hz), 6.88 (1H, dd, J = 9.0, 2.4 |
| | Hz), 6.95 (1H, dd, J = 5.3, 1.4 Hz), 7.54 (1H, d, J = 5.9 Hz), 7.87 (1H, d, J = 9.1 Hz), |
| | 8.05 (1H, d, J = 5.2 Hz) |
| 4270 | 1.55-1.69 (4H, m), 1.92-1.99 (1H, m), 2.08-2.12 (2H, m), 2.14 (3H, s), 2.27-2.33 |
| | (2H, m), 2.38 (2H, dd, J = 11.2, 3.6 Hz), 4.37 (2H, d, J = 6.2 Hz), 4.51 (2H, d, J = 7.5 |
| | Hz), 6.32 (2H, s), 6.43 (1H, d, J = 2.4 Hz), 6.53 (1H, d, J = 5.8 Hz), 6.75 (1H, s), 6.83 |
| | (1H, t, J = 6.3 Hz), 6.88 (1H, dd, J = 9.0, 2.4 Hz), 6.97 (1H, dd, J = 5.3, 1.4 Hz), 7.54 |
| | (1H, d, J = 5.8 Hz), 7.87 (1H, d, J = 9.1 Hz), 8.09 (1H, d, J = 5.3 Hz) |
| 4271 | 1.19-1.29 (2H, m), 1.59-1.69 (3H, m), 1.77-1.84 (2H, m), 2.13 (3H, s), 2.71-2.76 |
| | (2H, m), 4.05 (2H, d, J = 6.2 Hz), 4.49 (2H, d, J = 6.3 Hz), 6.32 (1H, s), 6.48 (1H, dd, |
| | J = 6.0, 0.8 Hz), 6.69 (1H, d, J = 1.4 Hz), 6.83 (2H, s), 6.92 (1H, dd, J = 5.3, 1.4 Hz), |
| | 7.35 (1H, t, J = 6.4 Hz), 7.63 (1H, d, J = 5.9 Hz), 8.02-8.05 (1H, m), 9.05 (1H, s) |
| 4272 | 1.19-1.28 (2H, m), 1.60-1.68 (3H, m), 1.77-1.84 (2H, m), 2.13 (3H, s), 2.71-2.75 |
| | (2H, m), 4.06 (2H, d, J = 6.1 Hz), 4.52 (2H, d, J = 6.1 Hz), 6.39 (1H, d, J = 5.4 Hz), 6.64 |
| | (2H, s), 6.71 (1H, s), 6.94 (1H, dd, J = 5.3, 1.4 Hz), 7.19 (1H, d, J = 6.1 Hz), 7.73 (1H, |
| | t, J = 6.3 Hz), 7.78 (1H, d, J = 6.0 Hz), 8.05-8.08 (1H, m), 8.23 (1H, d, J = 5.3 Hz) |
| 4273 | 1.18-1.28 (2H, m), 1.59-1.68 (3H, m), 1.77-1.83 (2H, m), 2.12 (3H, s), 2.70-2.75 |
| | (2H, m), 4.04 (2H, d, J = 6.2 Hz), 4.65 (2H, d, J = 6.4 Hz), 6.64 (1H, s), 6.66 (1H, d, J = |
| | 5.2 Hz), 6.77 (2H, s), 6.91 (1H, dd, J = 5.3, 1.4 Hz), 7.07 (1H, d, J = 6.0 Hz), 7.68 (1H, |
| | d, J = 6.0 Hz), 7.94 (1H, t, J = 6.4 Hz), 8.01 (1H, d, J = 5.3 Hz), 8.24 (1H, d, J = 5.2 Hz) |
| 4274 | 1.04-1.09 (1H, m, minor), 1.13-1.19 (1H, m, major), 1.49-1.54 (1H, m, minor), |
| | 1.56-1.63 (1H, m), 1.68-1.73 (1H, m, major), 1.81 (3H, s, minor), 1.92 (3H, s, |
| | major), 1.78-1.96 (1H, m), 2.40-2.48 (1H, m), 2.52-2.61 (1H, m), 2.98-3.03 (1H, |
| | m, major), 3.22-3.27 (1H, m), 3.29-3.37 (1H, m, minor), 4.02-4.24 (2H, m), 4.30- |
| | 4.36 (1H, m), 4.45 (2H, d, J = 6.0 Hz), 6.38 (1H, d, J = 7.7 Hz), 6.53 (2H, s), 6.72 (1H, |
| | s), 6.78-6.83 (1H, m), 6.98 (1H, dd, J = 5.2, 1.4 Hz), 7.09-7.14 (1H, m), 7.20 (1H, d, |
| | J = 6.1 Hz), 7.34 (1H, d, J = 8.3 Hz), 7.77 (1H, d, J = 6.0 Hz), 8.06 (1H, t, J = 5.1 Hz) |
| 4275 | 1.05-1.11 (1H, m, minor), 1.15-1.20 (1H, m, major), 1.50-1.55 (1H, m, minor), |
| | 1.58-1.63 (1H, m), 1.69-1.73 (1H, m, major), 1.82 (3H, s, minor), 1.92 (3H, s, |
| | major), 1.78-1.96 (1H, m), 2.40-2.49 (1H, m), 2.54-2.62 (1H, m), 2.99-3.03 (1H, |
| | m, minor), 3.23-3.28 (2 × H, m, major), 3.34-3.38 (1H, m, minor), 4.03-4.17 (1H, |
| | m and 1H, m, minor), 4.20-4.26 (1H, m, major), 4.32-4.36 (1H, m), 4.38 (2H, d, J = |
| | 6.4 Hz), 6.31 (2H, s), 6.42-6.43 (1H, m), 6.53 (1H, dd, J = 5.9, 2.3 Hz), 6.75 (1H, s), |
| | 6.82-6.86 (1H, m), 6.88 (1H, dd, J = 9.0, 2.3 Hz), 6.98 (1H, dd, J = 5.3, 1.4 Hz), 7.54 |
| | (1H, d, J = 5.8 Hz), 7.87 (1H, d, J = 9.0 Hz), 8.06-8.09 (1H, m) |
| 4276 | 1.11-1.17 (1H, m), 1.26-1.31 (1H, m), 1.55-1.66 (2H, m), 2.19 (3H, s), 2.20-2.31 |
| | (2H, m), 2.31-2.35 (1H, m), 2.55-2.60 (1H, m), 2.91-2.95 (1H, m), 4.11-4.17 (1H, |
| | m), 4.28 (1H, dd, J = 10.8, 6.7 Hz), 4.44 (2H, d, J = 6.0 Hz), 6.38 (1H, d, J = 7.7 Hz), |
| | 6.52 (2H, s), 6.68-6.72 (1H, m), 6.80 (1H, t, J = 6.1 Hz), 6.96 (1H, dd, J = 5.3, 1.4 |
| | Hz), 7.09-7.14 (1H, m), 7.20 (1H, d, J = 6.1 Hz), 7.33 (1H, d, J = 8.3 Hz), 7.77 (1H, d, |
| | J = 6.1 Hz), 8.05 (1H, d, J = 5.3 Hz) |
| 4277 | 1.11-1.18 (1H, m), 1.27-1.32 (1H, m), 1.56-1.67 (2H, m), 2.19 (3H, s), 2.22-2.32 |
| | (2H, m), 2.32-2.36 (1H, m), 2.56-2.61 (1H, m), 2.91-2.96 (1H, m), 4.11-4.19 (1H, |
| | m), 4.30 (1H, dd, J = 10.8, 6.7 Hz), 4.37 (2H, d, J = 6.2 Hz), 6.32 (2H, s), 6.43 (1H, d, |
| | J = 2.4 Hz), 6.53 (1H, dd, J = 5.9, 0.7 Hz), 6.72-6.76 (1H, m), 6.83 (1H, t, J = 6.3 Hz), |
| | 6.88 (1H, dd, J = 9.0, 2.4 Hz), 6.97 (1H, dd, J = 5.3, 1.4 Hz), 7.54 (1H, d, J = 5.8 Hz), |
| | 7.87 (1H, d, J = 9.1 Hz), 8.05-8.08 (1H, m) |
| 4278 | 1.56-1.70 (4H, m), 1.91-1.99 (1H, m), 2.06-2.11 (2H, m), 2.12-2.17 (3H, m), |
| | 2.27-2.31 (2H, m), 2.35-2.39 (2H, m), 4.44 (2H, d, J = 6.0 Hz), 4.50 (2H, d, J = 7.5 |
| | Hz), 6.39 (1H, d, J = 7.7 Hz), 6.52 (2H, s), 6.72 (1H, s), 6.80 (1H, t, J = 6.1 Hz), 6.97 |
| | (1H, dd, J = 5.3, 1.4 Hz), 7.09-7.14 (1H, m), 7.20 (1H, d, J = 6.1 Hz), 7.33 (1H, d, J = |
| | 8.3 Hz), 7.77 (1H, d, J = 6.1 Hz), 8.07 (1H, d, J = 5.2 Hz) |
| 4279 | 2.73 (3H, s), 3.33-3.36 (1H, m), 3.64 (1H, t, J = 8.9 Hz), 4.30-4.43 (2H, m), 4.45 |
| | (2H, d, J = 6.0 Hz), 4.75-4.85 (1H, m), 6.38 (1H, d, J = 7.7 Hz), 6.52 (2H, s), 6.78- |
| | 6.85 (2H, m), 7.01 (1H, dd, J = 5.3, 1.4 Hz), 7.11 (1H, t, J = 8.0 Hz), 7.17-7.21 (1H, |
| | m), 7.33 (1H, d, J = 8.3 Hz), 7.76 (1H, d, J = 6.1 Hz), 8.06 (1H, dd, J = 5.3, 0.7 Hz) |
| 4280 | 2.73 (3H, s), 3.33-3.34 (1H, m), 3.64 (1H, t, J = 9.0 Hz), 4.28-4.44 (2H, m), 4.45 |
| | (2H, d, J = 6.0 Hz), 4.75-4.84 (1H, m), 6.38 (1H, d, J = 7.5 Hz), 6.52 (2H, s), 6.77- |
| | 6.86 (2H, m), 7.01 (1H, dd, J = 5.3, 1.4 Hz), 7.12 (1H, t, J = 8.0 Hz), 7.17-7.22 (1H, |
| | m), 7.33 (1H, d, J = 8.3 Hz), 7.76 (1H, d, J = 6.1 Hz), 8.06 (1H, dd, J = 5.2, 0.6 Hz) |
| 4281 | 1.23-1.33 (2H, m), 1.69 (3H, d, J = 11.3 Hz), 1.96 (2H, t, J = 11.0 Hz), 2.20 (3H, s), |
| | 2.82 (2H, d, J = 11.2 Hz), 4.09 (2H, d, J = 6.0 Hz), 4.54 (2H, d, J = 5.8 Hz), 6.41 (1H, d, |
| | J = 7.7 Hz), 6.60 (2H, br.s), 6.86 (1H, t, J = 6.1 Hz), 7.01 (1H, s), 7.14 (1H, t, J = 8.0 |
| | Hz), 7.19 (1H, d, 6.1 Hz), 7.36 (1H, d, J = 8.3 Hz), 7.48 (1H, s), 7.78 (1H, d, J = 6.0 |
| | Hz), 8.21 (2H, br.s) |
| 4282 | 3.03 (2H, t, J = 7.0 Hz), 3.55 (3H, s), 4.44 (2H, d, J = 6.0 Hz), 4.50 (2H, t, J = 7.0 Hz), |
| | 6.37 (1H, dd, J = 7.8, 0.8 Hz), 6.52 (2H, s), 6.71 (1H, t, J = 1.1 Hz), 6.73 (1H, d, J = 1.2 |
| | Hz), 6.81 (1H, t, J = 6.2 Hz), 6.97 (1H, dd, J = 5.3, 1.4 Hz), 7.00 (1H, d, J = 1.2 Hz), |
| | 7.11 (1H, t, J = 8.0 Hz), 7.16-7.22 (1H, m), 7.33 (1H, d, J = 8.3 Hz), 7.76 (1H, d, J = |
| | 6.1 Hz), 8.06 (1H, dd, J = 5.2, 0.7 Hz) |
| 4283 | 3.04 (2H, t, J = 7.0 Hz), 3.55 (3H, s), 4.37 (2H, d, J = 6.2 Hz), 4.52 (2H, t, J = 7.0 Hz), |
| | 6.35 (2H, s), 6.42 (1H, d, J = 2.4 Hz), 6.52 (1H, dd, J = 5.9, 0.8 Hz), 6.73 (2H, d, J = 1.2 |
| | Hz), 6.83-6.89 (2H, m), 6.98 (1H, dd, J = 5.3, 1.4 Hz), 7.00 (1H, d, J = 1.2 Hz), 7.53 |
| | (1H, d, J = 5.8 Hz), 7.87 (1H, d, J = 9.0 Hz), 8.08 (1H, dd, J = 5.3, 0.7 Hz) |
| 4284 | 1.06-1.16 (2H, m), 1.35-1.46 (1H, m), 1.56-1.64 (2H, m), 1.67-1.76 (2H, m), |
| | 2.10 (3H, s), 2.65-2.72 (2H, m), 3.06 (2H, t, J = 6.3 Hz), 4.22 (2H, d, J = 5.9 Hz), 6.30 |
| | (2H, s), 6.40-6.43 (2H, m), 6.44 (1H, dd, J = 5.3, 1.4 Hz), 6.48 (1H, t, J = 5.7 Hz), |
| | 6.53 (1H, d, J = 5.9 Hz), 6.75 (1H, t, J = 6.1 Hz), 6.86 (1H, dd, J = 9.1, 2.3 Hz), 7.54 |
| | (1H, d, J = 5.8 Hz), 7.84-7.88 (2H, m) |
| 4285 | 1.03-1.17 (2H, m), 1.35-1.46 (1H, m), 1.59 (2H, d, J = 10.7 Hz), 1.70 (2H, td, J = |
| | 11.5, 2.6 Hz), 2.09 (3H, s), 2.63-2.72 (2H, m), 3.05 (2H, d, J = 6.3 Hz), 4.25 (2H, d, |
| | J = 6.0 Hz), 6.42 (1H, s), 6.44 (1H, dd, J = 5.2, 1.6 Hz), 6.49 (1H, t, J = 5.8 Hz), 6.54 |
| | (2H, s), 6.64 (1H, d, J = 2.4 Hz), 6.91 (1H, dd, J = 9.0, 2.4 Hz), 7.12 (1H, d, J = 6.1 Hz), |
| | 7.64 (1H, s), 7.86 (1H, d, J = 5.3 Hz), 7.92 (1H, d, J = 9.0 Hz) |
| 4286 | 1.00-1.15 (2H, m), 1.33-1.45 (1H, m), 1.58 (2H, d, J = 12.7 Hz), 1.64-1.77 (2H, |
| | m), 2.09 (3H, s), 2.68 (2H, d, J = 11.4 Hz), 3.03 (2H, t, J = 6.3 Hz), 4.30 (2H, d, J = 5.8 |
| | Hz), 6.32-6.46 (4H, m), 6.49 (2H, s), 6.72 (1H, t, J = 6.0 Hz), 7.12 (1H, t, J = 8.0 Hz), |
| | 7.19 (1H, d, J = 6.1 Hz), 7.31 (1H, d, J = 8.3 Hz), 7.75 (1H, d, J = 6.1 Hz), 7.84 (1H, d, |
| | J = 5.3 Hz) |
| 4287 | 1.72-1.84 (4H, m), 2.64-2.97 (4H, m), 3.92 (2H, s), 4.45 (2H, d, J = 5.9 Hz), 6.66- |
| | 6.72 (1H, m), 6.78 (1H, d, J = 6.9 Hz), 7.06 (1H, dd, J = 9.2, 2.3 Hz), 7.26-7.32 (1H, |
| | m), 7.33-7.39 (2H, m), 7.40-7.46 (2H, m), 7.54-7.61 (1H, m), 8.03 (2H, s), 8.12 |
| | (1H, d, J = 9.2 Hz), 11.40 (2H, br. s) |
| 4288 | 2.13 (3H, s), 2.22-2.32 (4H, m), 2.38-2.40 (4H, m), 2.45-2.48 (2H, m), 2.67-2.73 |
| | (2H, m), 4.32 (2H, d, J = 5.9 Hz), 6.28 (2H, s), 6.48 (1H, d, J = 2.3 Hz), 6.53 (1H, d, J = |
| | 5.9 Hz), 6.70 (1H, t, J = 6.0 Hz), 6.88 (1H, dd, J = 9.0, 2.3 Hz), 7.09 (1H, d, J = 7.2 Hz), |
| | 7.17-7.26 (3H, m), 7.53 (1H, d, J = 5.8 Hz), 7.84 (1H, d, J = 9.0 Hz). |
| 4289 | 1.59-1.72 (4H, m), 1.95-2.01 (2H, m), 2.16 (3H, s), 2.66-2.74 (2H, m), 3.68-3.77 |
| | (1H, m), 4.57 (2H, d, J = 5.8 Hz), 6.34 (1H, d, J = 7.6 Hz), 6.54 (2H, s), 7.09 (1H, t, J = |
| | 8.0 Hz), 7.22 (1H, d, J = 6.2 Hz), 7.33 (1H, d, J = 8.3 Hz), 7.56 (1H, dd, J = 5.0, 1.4 Hz), |
| | 7.78 (1H, d, J = 6.0 Hz), 8.03 (1H, d, J = 0.6 Hz), 8.22 (1H, s), 8.48 (1H, d, J = 8.4 Hz), |
| | 8.54 (1H, d, J = 5.0 Hz) |
| 4293 | 1.19-1.29 (2H, m), 1.60-1.69 (3H, m), 1.77-1.84 (2H, m), 2.13 (3H, s), 2.71-2.77 |
| | (2H, m), 4.07 (2H, d, J = 6.1 Hz), 4.40 (2H, d, J = 6.1 Hz), 6.47 (2H, s), 6.58 (1H, d, J = |
| | 5.8 Hz), 6.73 (1H, d, J = 2.7 Hz), 6.77 (1H, s), 6.97 (1H, dd, J = 5.3, 1.4 Hz), 7.21 (1H, |
| | t, J = 6.2 Hz), 7.62 (1H, d, J = 5.7 Hz), 8.08 (1H, d, J = 5.2 Hz), 8.36 (1H, d, J = 2.7 Hz) |
| 4294 | 0.85 (3H, d, J = 6.1 Hz), 0.89 (3H, d, J = 6.0 Hz), 1.41-1.55 (2H, m), 2.27 (1H, d, J = |
| | 9.4 Hz), 2.32 (1H, dd, J = 9.7, 2.3 Hz), 2.35-2.45 (1H, m), 2.56-2.66 (2H, m), 3.04 |
| | (1H, s), 3.62 (1H, d, J = 14.9 Hz), 3.74 (1H, d, J = 14.9 Hz), 4.50 (2H, d, J = 5.9 Hz), |
| | 6.33 (1H, d, J = 7.7 Hz), 6.50 (2H, s), 6.84 (1H, t, J = 6.1 Hz), 7.08 (1H, t, J = 8.0 Hz), |
| | 7.18-7.24 (2H, m), 7.31 (1H, d, J = 8.3 Hz), 7.43 (1H, s), 7.76 (1H, d, J = 6.1 Hz), |
| | 8.35 (1H, d, J = 5.1 Hz). One proton obscured by water peak |
| 4295 | CDCl₃ 1.03 (3H, d, J = 6.3 Hz), 1.08 (3H, d, J = 6.1 Hz), 1.79 (2H, s), 2.57 (2H, dd, J = |
| | 10.3, 2.4 Hz), 2.61-2.70 (1H, m), 2.86-2.96 (2H, m), 3.22 (1H, s), 3.61 (1H, s), |
| | 3.76-3.82 (1H, m), 3.91 (1H, d, J = 14.6 Hz), 4.56 (2H, d, J = 5.6 Hz), 4.86 (1H, d, J = |
| | 5.8 Hz), 5.11 (2H, s), 6.56 (1H, d, J = 7.6 Hz), 7.03 (1H, dd, J = 6.3, 1.0 Hz), 7.16 (1H, |
| | dt, J = 8.4, 1.0 Hz), 7.19 (1H, dd, J = 5.1, 1.7 Hz), 7.29 (1H, d, J = 8.1 Hz), 7.49 (1H, d, |
| | J = 1.7 Hz), 7.97 (1H, d, J = 6.1 Hz), 8.48 (1H, dd, J = 5.1, 0.8 Hz). |
| 4296 | CDCl₃ 1.13 (3H, s), 1.14 (3H, s), 2.00-2.09 (4H, m), 2.42-2.54 (2H, m), 2.83-2.89 |
| | (2H, m), 2.92-3.00 (1H, m), 3.46-3.54 (1H, m), 4.56 (2H, d, J = 5.9 Hz), 4.64 (2H, |
| | s), 4.86 (1H, s), 5.04 (2H, s), 6.88-6.95 (2H, m), 7.22 (1H, d, J = 5.2 Hz), 7.48 (1H, s), |
| | 7.63 (1H, d, J = 8.9 Hz), 7.84 (1H, s), 8.51 (1H, d, J = 5.2 Hz) |
| 4297 | CDCl₃ 0.82-0.91 (3H, m), 1.24-1.29 (6H, m), 2.06-2.17 (4H, m), 2.94 (2H, d, J = |
| | 11.8 Hz), 3.15 (2H, d, J = 12.7 Hz), 3.26-3.32 (1H, m), 4.51 (2H, s), 4.59 (2H, d, J = |
| | 7.8 Hz), 6.74 (1H, d, J = 7.9 Hz), 6.77 (1H, s), 6.93 (1H, dd, J = 5.3, 1.4 Hz), 6.98 (1H, |
| | dd, J = 6.9, 0.9 Hz), 7.23-7.26 (1H, m), 7.41 (1H, app.t, J = 8.1 Hz), 7.63 (1H, d, J = |
| | 6.8 Hz), 7.93 (2H, br.s), 8.13 (1H, d, J = 0.6 Hz), 8.51 (2H, s) (NH2 weak and broad at |
| | 7.93 ppm, but amide NH not observed, 2 formic acid salt) |
| 4298 | 1.57-1.77 (1H, m), 2.05-2.15 (1H, m), 2.26-2.40 (1H, m), 2.41-2.47 (1H, m), |
| | 2.89 (1H, ddd, J = 16.2, 5.0, 1.4 Hz), 3.85 (1H, td, J = 11.9, 4.7 Hz), 4.00-4.09 (1H, |
| | m), 4.22 (2H, dd, J = 6.6, 1.6 Hz), 4.45 (2H, d, J = 5.9 Hz), 6.38 (1H, dd, J = 7.8, 0.9 |
| | Hz), 6.51 (2H, s), 6.76-6.78 (1H, m), 6.78 (1H, d, J = 1.2 Hz), 6.81 (1H, t, J = 6.1 Hz), |
| | 6.97 (1H, d, J = 1.2 Hz), 6.98 (1H, dd, J = 5.3, 1.4 Hz), 7.12 (1H, t, J = 8.0 Hz), 7.20 |
| | (1H, dd, J = 6.3, 0.9 Hz), 7.33 (1H, d, J = 8.3 Hz), 7.76 (1H, d, J = 6.1 Hz), 8.06 (1H, |
| | dd, J = 5.3, 0.7 Hz) |
| 4299 | 0.93 (6H, d, J = 6.6 Hz), 1.13-1.23 (2H, m), 1.59-1.68 (3H, m), 2.02-2.08 (2H, m), |
| | 2.60-2.67 (1H, m), 2.74 (2H, d, J = 11.7 Hz), 4.02 (2H, d, J = 6.2 Hz), 4.43 (2H, d, J = |
| | 5.9 Hz), 6.37 (1H, d, J = 7.7 Hz), 6.51 (2H, s), 6.71 (1H, s), 6.79 (1H, t, J = 6.1 Hz), |
| | 6.95 (1H, dd, J = 5.4, 0.8 Hz), 7.11 (1H, t, J = 7.9 Hz), 7.19 (1H, d, J = 6.2 Hz), 7.33 |
| | (1H, d, J = 8.3 Hz), 7.76 (1H, d, J = 6.0 Hz), 8.03 (1H, d, J = 5.3 Hz) |
| 4300 | 1.61-1.77 (1H, m), 2.01-2.18 (1H, m), 2.27-2.40 (1H, m), 2.43-2.48 (1H, m), |
| | 2.90 (1H, dd, J = 16.2, 4.9 Hz), 3.86 (1H, td, J = 12.0, 4.8 Hz), 4.06 (1H, ddd, J = 12.5, |
| | 5.5, 2.8 Hz), 4.24 (2H, d, J = 6.5 Hz), 4.42 (2H, d, J = 6.1 Hz), 6.57 (2H, s), 6.65 (1H, d, |
| | J = 2.3 Hz), 6.79 (1H, d, J = 1.3 Hz), 6.82 (1H, s), 6.95 (1H, dd, J = 9.1, 2.4 Hz), 6.97 |
| | (1H, d, J = 1.2 Hz), 7.00 (1H, dd, J = 5.3, 1.4 Hz), 7.21 (1H, t, J = 6.1 Hz), 7.64 (1H, s), |
| | 7.94 (1H, d, J = 9.1 Hz), 8.09 (1H, dd, J = 5.3, 0.7 Hz) |
| 4301 | 3.37 (3H, s), 4.47 (2H, d, J = 6.0 Hz), 5.17 (2H, d, J = 1.2 Hz), 6.18 (1H, dd, J = 6.9, 1.9 |
| | Hz), 6.29 (1H, d, J = 1.7 Hz), 6.40 (1H, d, J = 7.7 Hz), 6.52 (2H, s), 6.82 (1H, t, J = 6.1 |
| | Hz), 6.87 (1H, s), 7.01 (1H, dd, J = 5.3, 1.4 Hz), 7.12 (1H, t, J = 8.0 Hz), 7.20 (1H, d, |
| | J = 6.1 Hz), 7.34 (1H, d, J = 8.3 Hz), 7.62 (1H, d, J = 7.0 Hz), 7.77 (1H, d, J = 6.0 Hz), |
| | 8.05 (1H, d, J = 5.4 Hz) |
| 4302 | 1.67-1.76 (2H, m), 1.76-1.85 (2H, m), 2.42 (2H, t, J = 7.0 Hz), 3.24 (2H, t, J = 6.8 |
| | Hz), 3.29 (2H, t, J = 6.7 Hz), 3.39 (2H, q, J = 6.7 Hz), 4.30 (2H, d, J = 5.8 Hz), 6.35 (1H, |
| | d, J = 7.7 Hz), 6.38-6.44 (2H, m), 6.47 (1H, dd, J = 5.3, 1.4 Hz), 6.50 (2H, s), 6.74 |
| | (1H, t, J = 6.0 Hz), 7.12 (1H, t, J = 8.0 Hz), 7.20 (1H, d, J = 6.1 Hz), 7.31 (1H, d, J = 8.3 |
| | Hz), 7.75 (1H, d, J = 6.1 Hz), 7.87 (1H, d, J = 5.2 Hz) |
| 4303 | 1.52-1.67 (1H, m), 2.02 (1H, d, J = 13.3 Hz), 2.06-2.22 (1H, m), 2.35 (1H, dd, J = |
| | 16.4, 10.5 Hz), 2.85 (1H, dd, J = 16.4, 5.1, 1.5 Hz), 3.18-3.28 (2H, m), 3.78 (1H, td, |
| | J = 11.8, 4.8 Hz), 3.98-4.08 (1H, m), 4.27 (2H, d, J = 6.0 Hz), 6.45-6.51 (2H, m), 6.55 |
| | (2H, s), 6.64-6.72 (2H, m), 6.77 (1H, d, J = 1.2 Hz), 6.92 (1H, dd, J = 9.1, 2.4 Hz), |
| | 6.94 (1H, d, J = 1.2 Hz), 7.15 (1H, t, J = 6.0 Hz), 7.64 (1H, s), 7.91 (2H, d) |
| 4306 | 2.21 (2H, q, J = 5.9 Hz), 2.92 (1H, dd, J = 16.8, 5.2 Hz), 3.14 (1H, dd, J = 16.8, 4.6 Hz), |
| | 4.00 (2H, hept, J = 6.7 Hz), 4.43 (2H, d, J = 5.9 Hz), 5.51 (1H, p, J = 4.8 Hz), 6.37 (1H, |
| | d, J = 7.6 Hz), 6.53 (2H, s), 6.72 (1H, s), 6.76-6.85 (2H, m), 6.96-7.03 (2H, m), 7.11 |
| | (1H, t, J = 8.0 Hz), 7.15-7.21 (1H, m), 7.32 (1H, d, J = 8.3 Hz), 7.75 (1H, d, J = 6.1 |
| | Hz), 8.09 (1H, d, J = 5.2 Hz) |
| 4307 | 1.64-1.77 (1H, m), 2.11 (1H, d, J = 13.3 Hz), 2.34-2.41 (1H, m), 2.86-2.94 (1H, m, |
| | in water peak), 3.86 (1H, td, J = 11.9, 4.7 Hz, in water peak), 4.02-4.10 (1H, m), |
| | 4.24 (2H, d, J = 6.5 Hz), 4.43 (2H, d, J = 6.1 Hz), 6.29 (2H, s), 6.51 (1H, d, J = 2.3 Hz), |
| | 6.79 (1H, d, J = 1.2 Hz), 6.81 (1H, s), 6.94-7.02 (3H, m), 7.15 (1H, t, J = 6.2 Hz), 7.52 |
| | (1H, d, J = 2.9 Hz), 7.92 (1H, dd, J = 9.2, 2.5 Hz), 8.09 (1H, d, J = 5.3 Hz), 8.19 (2H, s). |
| | One aliphatic proton obscured by DMSO peak, 2 acidic protons not seen. |
| 4308 | 1.69 (1H, qd, J = 11.7, 5.6 Hz), 2.10 (1H, d, J = 13.4 Hz), 2.23-2.40 (1H, m), 2.41- |
| | 2.48 (1H, m), 2.89 (1H, dd, J = 16.3, 4.9 Hz), 3.85 (1H, td, J = 11.9, 4.6 Hz), 4.00- |
| | 4.09 (1H, m), 4.23 (2H, d, J = 6.5 Hz), 4.48 (2H, d, J = 5.7 Hz), 6.45 (2H, s), 6.47 (1H, |
| | s), 6.72 (1H, dt, J = 13.1, 5.9 Hz), 6.77-6.82 (2H, m), 6.95-6.98 (1H, m), 6.98-7.03 |
| | (1H, m), 7.23 (1H, t, J = 8.0 Hz), 7.25-7.36 (1H, m), 7.64 (1H, d, J = 5.7 Hz), 8.07 |
| | (1H, d, J = 5.3 Hz) |
| 4309 | 1.66-1.79 (1H, m), 2.09 (4H, s), 2.31-2.42 (1H, m), 2.53-2.60 (1H, m), 2.94 (1H, |
| | dd, J = 16.7, 5.1 Hz), 3.86 (1H, td, J = 12.4, 12.0, 4.7 Hz), 4.01-4.08 (1H, m), 4.25 |
| | (2H, d, J = 6.5 Hz), 4.43 (2H, d, J = 6.1 Hz), 6.61 (2H, s), 6.65 (1H, d, J = 2.3 Hz), 6.80 |
| | (1H, s), 6.88 (1H, s), 6.95 (1H, dd, J = 9.1, 2.4 Hz), 7.01 (1H, dd, J = 5.2, 1.4 Hz), 7.24 |
| | (1H, t, J = 6.2 Hz), 7.65 (1H, s), 7.95 (1H, d, J = 9.1 Hz), 8.09 (1H, d, J = 5.2 Hz) |
| 4319 | 1.65-1.75 (1H, m), 2.09 (3H, s), 2.12 (1H, d, J = 4.0 Hz), 2.31-2.43 (1H, m), 2.53- |
| | 2.59 (1H, m), 2.94 (1H, dd, J = 16.7, 5.1 Hz), 3.86 (1H, td, J = 12.4, 12.0, 4.7 Hz), |
| | 4.00-4.10 (1H, m), 4.25 (2H, d, J = 6.5 Hz), 4.43 (2H, d, J = 6.1 Hz), 6.61 (2H, s), |
| | 6.65 (1H, d, J = 2.3 Hz), 6.80 (1H, s), 6.88 (1H, s), 6.95 (1H, dd, J = 9.1, 2.4 Hz), 7.01 |
| | (1H, dd, J = 5.2, 1.4 Hz), 7.24 (1H, t, J = 6.2 Hz), 7.65 (1H, s), 7.95 (1H, d, J = 9.1 Hz), |
| | 8.09 (1H, d, J = 5.2 Hz) |
| 4320 | 1.60-1.78 (1H, m), 2.08 (3H, s), 2.09-2.19 (1H, m), 2.21-2.34 (1H, m), 2.43 (1H, |
| | dd, J = 16.1, 10.8 Hz), 2.84 (1H, dd, J = 16.1, 4.9, 1.5 Hz), 3.66 (1H, td, J = 11.8, 4.9 |
| | Hz), 3.85-3.95 (1H, m), 4.15-4.27 (2H, m), 4.45 (2H, d, J = 5.9 Hz), 6.38 (1H, d, J = |
| | 7.6 Hz), 6.50 (1H, d, J = 1.2 Hz), 6.54 (2H, s), 6.74-6.80 (1H, m), 6.83 (1H, t, J = 6.1 |
| | Hz), 6.98 (1H, dd, J = 5.3, 1.4 Hz), 7.12 (1H, t, J = 8.0 Hz), 7.20 (1H, d, J = 6.1 Hz), |
| | 7.33 (1H, d, J = 8.3 Hz), 7.77 (1H, d, J = 6.0 Hz), 8.06 (1H, d, J = 5.3 Hz) |
| 4408 | 1.70 (1H, dtd, J = 13.3, 11.3, 5.6 Hz), 2.07-2.14 (1H, m), 2.31-2.40 (1H, m), 2.43- |
| | 2.49 (1H, m), 2.86-2.94 (1H, m), 3.87 (1H, td, J = 12.0, 4.7 Hz), 4.06 (1H, ddd, J = |
| | 12.6, 5.6, 2.8 Hz), 4.24 (2H, d, J = 6.6 Hz), 4.39 (2H, d, J = 6.1 Hz), 6.31 (2H, s), 6.44 |
| | (1H, d, J = 2.3 Hz), 6.53 (1H, d, J = 5.9 Hz), 6.78-6.82 (2H, m), 6.85 (1H, t, J = 6.2 |
| | Hz), 6.88 (1H, dd, J = 9.0, 2.3 Hz), 6.95-7.09 (2H, m), 7.54 (1H, d, J = 5.8 Hz), 7.87 |
| | (1H, d, J = 9.0 Hz), 8.08 (1H, d, J = 5.3 Hz) |
| 4409 | 1.06-1.14 (3H, m), 1.51-1.62 (4H, m), 1.68 (2H, t, J = 10.8 Hz), 2.08 (3H, s), 2.62- |
| | 2.72 (4H, m), 4.38 (2H, d, J = 5.9 Hz), 6.32 (2H, s), 6.41 (1H, d, J = 2.3 Hz), 6.52 (1H, |
| | d, J = 5.9 Hz), 6.84 (1H, t, J = 6.2 Hz), 6.88 (1H, dd, J = 9.1, 2.4 Hz), 7.17 (1H, dd, J = |
| | 5.2, 1.6 Hz), 7.23 (1H, s), 7.53 (1H, d, J = 5.8 Hz), 7.87 (1H, d, J = 9.0 Hz), 8.38 (1H, d, |
| | J = 5.1 Hz) |
| 4410 | 1.07-1.15 (3H, m), 1.43-1.50 (2H, m), 1.58-1.64 (2H, m), 1.67-1.74 (2H, m), |
| | 2.09 (3H, s), 2.53-2.59 (2H, m), 2.65-2.70 (2H, m), 4.32 (2H, d, J = 5.6 Hz), 6.29 |
| | (2H, s), 6.47 (1H, d, J = 2.3 Hz), 6.53 (1H, d, J = 5.8 Hz), 6.72 (1H, t, J = 5.9 Hz), 6.88 |
| | (1H, dd, J = 9.1, 2.2 Hz), 7.06 (1H, d, J = 7.4 Hz), 7.16-7.26 (3H, m), 7.53 (1H, d, J = |
| | 5.8 Hz), 7.84 (1H, d, J = 9.0 Hz) |
| 4411 | 1.57-1.80 (1H, m), 2.12 (1H, d, J = 13.6 Hz), 2.34-2.43 (1H, m), 2.57 (1H, dd, J = |
| | 16.5, 10.8 Hz), 3.05 (1H, dd, J = 16.5, 5.0 Hz), 3.83-3.96 (1H, m), 4.18 (1H, ddd, J = |
| | 12.6, 5.6, 2.8 Hz), 4.21-4.33 (2H, m), 4.39 (2H, d, J = 6.0 Hz), 6.31 (2H, d, J = 6.2 |
| | Hz), 6.43 (1H, d, J = 2.4 Hz), 6.53 (1H, d, J = 5.9 Hz), 6.81 (1H, s), 6.85 (1H, t, J = 6.2 |
| | Hz), 6.88 (1H, dd, J = 9.0, 2.4 Hz), 7.00 (1H, dd, J = 5.2, 1.4 Hz), 7.54 (1H, d, J = 5.8 |
| | Hz), 7.87 (1H, d, J = 9.1 Hz), 8.09 (1H, d, J = 5.3 Hz), 8.36 (1H, s) |
| 4412 | 1.64-1.76 (1H, m), 2.11 (1H, d, J = 12.9 Hz), 2.36 (1H, s), 2.45-2.50 (1H, m), 2.85- |
| | 2.96 (1H, m), 3.87 (1H, td, J = 11.9, 4.7 Hz), 4.06 (1H, ddd, J = 12.6, 5.6, 2.8 Hz), |
| | 4.24 (2H, d, J = 6.5 Hz), 4.39 (2H, d, J = 6.0 Hz), 6.26-6.35 (2H, m), 6.44 (1H, d, J = |
| | 2.3 Hz), 6.53 (1H, d, J = 5.8 Hz), 6.80 (1H, d, J = 1.2 Hz), 6.81 (1H, s), 6.85 (1H, t, J = |
| | 6.3 Hz), 6.88 (1H, dd, J = 9.0, 2.3 Hz), 6.96-7.04 (2H, m), 7.54 (1H, d, J = 5.8 Hz), |
| | 7.87 (1H, d, J = 9.0 Hz), 8.08 (1H, d, J = 5.3 Hz) |
| 4413 | 1.66-1.77 (1H, m), 2.10-2.16 (1H, m), 2.26 (3H, s), 2.29-2.36 (1H, m), 2.53-2.57 |
| | (1H, m), 2.96-3.03 (1H, m), 3.69-3.78 (1H, m), 3.96-4.03 (1H, m), 4.22-4.29 (2H, |
| | m), 4.39 (2H, d, J = 6.2 Hz), 6.31 (2H, s), 6.43 (1H, d, J = 2.3 Hz), 6.53 (1H, d, J = 5.9 |
| | Hz), 6.81 (1H, s), 6.83-6.87 (1H, m), 6.87-6.91 (1H, m), 7.00 (1H, dd, J = 5.3, 1.4 |
| | Hz), 7.54 (1H, d, J = 5.8 Hz), 7.87 (1H, d, J = 9.0 Hz), 8.08 (1H, d, J = 5.3 Hz) |
| 4414 | 1.17 (3H, d, J = 6.3 Hz), 1.20-1.26 (1H, m), 1.26-1.33 (1H, m), 1.41-1.50 (1H, m), |
| | 1.53-1.60 (1H, m), 1.63-1.71 (1H, m), 1.71-1.81 (2H, m), 2.11 (3H, s), 2.69-2.80 |
| | (2H, m), 4.36 (2H, d, J = 6.2 Hz), 4.94-5.03 (1H, m), 6.31 (2H, s), 6.44 (1H, d, J = 2.4 |
| | Hz), 6.53 (1H, d, J = 5.9 Hz), 6.70 (1H, s), 6.81 (1H, t, J = 6.2 Hz), 6.88 (1H, dd, J = |
| | 9.0, 2.4 Hz), 6.93 (1H, dd, J = 5.3, 1.4 Hz), 7.54 (1H, d, J = 5.8 Hz), 7.87 (1H, d, J = 9.1 |
| | Hz), 8.05 (1H, d, J = 5.2 Hz) |
| 4415 | 1.67-1.80 (2H, m), 1.81-1.89 (2H, m), 2.09-2.20 (5H, m), 2.52-2.59 (2H, m), |
| | 4.31 (2H, d, J = 21.4 Hz), 4.39 (2H, d, J = 6.1 Hz), 6.31 (2H, s), 6.43 (1H, d, J = 2.4 Hz), |
| | 6.53 (1H, d, J = 5.9 Hz), 6.82 (1H, s), 6.85 (1H, t, J = 6.2 Hz), 6.88 (1H, dd, J = 9.1, 2.4 |
| | Hz), 7.01 (1H, dd, J = 5.3, 1.4 Hz), 7.54 (1H, d, J = 5.9 Hz), 7.87 (1H, d, J = 9.0 Hz), |
| | 8.07 (1H, d, J = 5.3 Hz) |
| 4416 | 1.49-1.59 (1H, m), 1.89-1.96 (1H, m), 1.99-2.07 (1H, m), 2.21-2.29 (1H, m), |
| | 2.30-2.36 (1H, m), 2.80 (3H, s), 3.24-3.29 (2H, m), 4.07-4.16 (2H, m), 4.38 (2H, |
| | d, J = 6.2 Hz), 6.32 (2H, s), 6.43 (1H, d, J = 2.4 Hz), 6.53 (1H, d, J = 5.8 Hz), 6.78 (1H, |
| | s), 6.84 (1H, t, J = 6.3 Hz), 6.88 (1H, dd, J = 9.0, 2.4 Hz), 6.98 (1H, dd, J = 5.2, 1.4 Hz), |
| | 7.54 (1H, d, J = 5.8 Hz), 7.87 (1H, d, J = 9.0 Hz), 8.07 (1H, d, J = 5.3 Hz) |
| 4417 | 1.48-1.58 (1H, m), 1.88-1.96 (1H, m), 1.98-2.07 (1H, m), 2.19-2.28 (1H, m), |
| | 2.28-2.35 (1H, m), 2.79 (3H, s), 3.23-3.29 (2H, m), 4.05-4.14 (2H, m), 4.45 (2H, |
| | d, J = 6.0 Hz), 6.38 (1H, d, J = 7.7 Hz), 6.52 (2H, s), 6.75 (1H, s), 6.81 (1H, t, J = 6.2 |
| | Hz), 6.98 (1H, dd, J = 5.3, 1.4 Hz), 7.09-7.15 (1H, m), 7.20 (1H, d, J = 6.1 Hz), 7.33 |
| | (1H, d, J = 8.3 Hz), 7.77 (1H, d, J = 6.1 Hz), 8.05 (1H, d, J = 5.2 Hz) |
| 4418 | 1.36-1.39 (2H, m), 1.47-1.53 (1H, m), 2.15-2.20 (5H, m), 2.88 (2H, d, J = 8.7 Hz), |
| | 4.01 (2H, d, J = 7.4 Hz), 4.44 (2H, d, J = 6.0 Hz), 6.38 (1H, d, J = 7.6 Hz), 6.52 (2H, s), |
| | 6.72 (1H, s), 6.80 (1H, t, J = 6.1 Hz), 6.95 (1H, dd, J = 5.2, 1.4 Hz), 7.09-7.16 (1H, |
| | m), 7.20 (1H, d, J = 6.0 Hz), 7.33 (1H, d, J = 8.3 Hz), 7.77 (1H, d, J = 6.1 Hz), 7.98- |
| | 8.08 (1H, m) |
| 4419 | 1.21-1.33 (2H, m), 1.65-1.73 (3H, m), 1.85-1.95 (2H, m), 2.18 (3H, s), 2.72 (3H, |
| | s), 2.75-2.83 (2H, m), 4.08 (2H, d, J = 6.1 Hz), 4.21 (2H, s), 6.71 (2H, s), 6.78 (1H, s), |
| | 6.96 (1H, dd, J = 5.3, 1.4 Hz), 7.11 (1H, dd, J = 6.0, 0.9 Hz), 7.26 (1H, dd, J = 7.6, 1.0 |
| | Hz), 7.32-7.37 (1H, m), 7.81 (1H, d, J = 6.0 Hz), 7.85 (1H, d, J = 8.3 Hz), 8.07 (1H, d, |
| | J = 5.2 Hz). |
| 4420 | 2.03-2.08 (2H, m), 2.71 (2H, t, J = 7.5 Hz), 3.51 (3H, s), 4.27 (2H, t, J = 6.4 Hz), 4.38 |
| | (2H, d, J = 6.1 Hz), 6.31 (2H, s), 6.42 (1H, d, J = 2.3 Hz), 6.53 (1H, d, J = 5.8 Hz), 6.71 |
| | (1H, d, J = 1.3 Hz), 6.77 (1H, s), 6.84 (1H, t, J = 6.3 Hz), 6.88 (1H, dd, J = 9.1, 2.4 Hz), |
| | 6.95-6.98 (2H, m), 7.54 (1H, d, J = 5.8 Hz), 7.87 (1H, d, J = 9.1 Hz), 8.07 (1H, d, J = |
| | 5.3 Hz) |
| 4421 | 2.01-2.09 (2H, m), 2.70 (2H, t, J = 7.5 Hz), 3.50 (3H, s), 4.25 (2H, t, J = 6.4 Hz), 4.45 |
| | (2H, d, J = 6.0 Hz), 6.38 (1H, d, J = 7.7 Hz), 6.52 (2H, s), 6.70 (1H, d, J = 1.2 Hz), 6.73 |
| | (1H, s), 6.81 (1H, t, J = 6.1 Hz), 6.94-6.98 (2H, m), 7.12 (1H, app t, J = 8.0 Hz), 7.20 |
| | (1H, d, J = 6.1 Hz), 7.34 (1H, d, J = 8.4 Hz), 7.77 (1H, d, J = 6.0 Hz), 8.04 (1H, d, J = |
| | 5.3 Hz) |
| 4422 | 3.66 (3H, s), 4.39 (2H, d, J = 6.1 Hz), 5.30 (2H, s), 6.36 (2H, s), 6.44 (1H, d, J = 2.3 |
| | Hz), 6.54 (1H, d, J = 5.9 Hz), 6.82 (1H, s), 6.84 (1H, d, J = 1.2 Hz), 6.88 (2H, dd, J = |
| | 9.1, 2.5 Hz), 7.03 (1H, dd, J = 5.3, 1.4 Hz), 7.16 (1H, d, J = 1.2 Hz), 7.53 (1H, d, J = 5.9 |
| | Hz), 7.88 (1H, d, J = 9.0 Hz), 8.13 (1H, d, J = 5.2 Hz) |
| 4423 | 3.65 (3H, s), 4.45 (2H, d, J = 6.0 Hz), 5.29 (2H, s), 6.39 (1H, d, J = 7.7 Hz), 6.51 (2H, |
| | s), 6.78-6.82 (2H, m), 6.84 (1H, d, J = 1.2 Hz), 7.03 (1H, dd, J = 5.3, 1.4 Hz), 7.12 |
| | (1H, t, J = 8.0 Hz), 7.15 (1H, d, J = 1.2 Hz), 7.19 (1H, d, J = 6.1 Hz), 7.33 (1H, d, J = 8.3 |
| | Hz), 7.76 (1H, d, J = 6.1 Hz), 8.11 (1H, d, J = 5.3 Hz) |
| 4424 | 0.95 (3H, s), 1.27-1.40 (2H, m), 1.49-1.60 (2H, m), 2.12-2.20 (5H, m), 2.37 (2H, |
| | d, J = 11.2 Hz), 3.98 (2H, s), 4.44 (2H, d, J = 6.0 Hz), 6.38 (1H, d, J = 7.7 Hz), 6.52 (2H, |
| | s), 6.74 (1H, s), 6.80 (1H, t, J = 6.1 Hz), 6.96 (1H, dd, J = 5.3, 1.4 Hz), 7.09-7.16 (1H, |
| | m), 7.20 (1H, d, J = 6.1 Hz), 7.34 (1H, d, J = 8.4 Hz), 7.77 (1H, d, J = 6.1 Hz), 8.04 (1H, |
| | d, J = 5.3 Hz) |
| 4425 | 0.96 (3H, s), 1.29-1.38 (2H, m), 1.52-1.61 (2H, m), 2.11-2.21 (5H, m), 2.33-2.43 |
| | (2H, m), 3.99 (2H, s), 4.37 (2H, d, J = 6.2 Hz), 6.31 (2H, s), 6.43 (1H, d, J = 2.4 Hz), |
| | 6.53 (1H, d, J = 5.8 Hz), 6.77 (1H, s), 6.83 (1H, t, J = 6.2 Hz), 6.88 (1H, dd, J = 9.0, 2.4 |
| | Hz), 6.97 (1H, dd, J = 5.3, 1.4 Hz), 7.54 (1H, d, J = 5.8 Hz), 7.87 (1H, d, J = 9.0 Hz), |
| | 8.06 (1H, d, J = 5.3 Hz) |
| 4426 | 2.19 (3H, s), 2.36 (4H, t, J = 5.0 Hz), 3.42 (4H, t, J = 5.0 Hz), 4.37 (2H, d, J = 5.9 Hz), |
| | 6.41 (1H, d, J = 7.6 Hz), 6.52 (2H, s), 6.64 (1H, dd, J = 5.1, 1.2 Hz), 6.74 (1H, t, J = 6.1 |
| | Hz), 6.84 (1H, s), 7.12 (1H, t, J = 8.0 Hz), 7.17-7.23 (1H, m), 7.32 (1H, d, J = 8.3 Hz), |
| | 7.76 (1H, d, J = 6.1 Hz), 7.99 (1H, d, J = 5.1 Hz) |
| 4427 | 2.19 (3H, s), 2.36 (4H, t, J = 5.0 Hz), 3.45 (4H, t, J = 5.1 Hz), 4.32 (2H, d, J = 6.0 Hz), |
| | 6.56 (2H, s), 6.65 (1H, dd, J = 5.1, 1.2 Hz), 6.67 (1H, d, J = 2.4 Hz), 6.86 (1H, s), 6.94 |
| | (1H, dd, J = 9.1, 2.3 Hz), 7.13 (1H, t, J = 6.1 Hz), 7.65 (1H, s), 7.92 (1H, d, J = 9.1 Hz), |
| | 8.03 (1H, d, J = 5.1 Hz). |
| 4428 | 1.68-1.83 (4H, m), 2.44 (2H, t, J = 6.9 Hz), 3.23-3.30 (4H, m), 3.38-3.45 (2H, m), |
| | 4.26 (2H, d, J = 6.0 Hz), 6.43-6.49 (3H, m), 6.55 (2H, s), 6.64 (1H, d, J = 2.1 Hz), |
| | 6.91 (1H, dd, J = 9.1, 2.3 Hz), 7.15 (1H, t, J = 6.2 Hz), 7.64 (1H, s), 7.89-7.93 (2H, m) |
| 4429 | 1.62-1.79 (1H, m), 2.10 (1H, d, J = 13.8 Hz), 2.27-2.41 (1H, m), 2.42-2.48 (1H, |
| | m), 2.90 (1H, dd, J = 16.2, 4.8 Hz), 3.85 (1H, dt, J = 12.0, 5.8 Hz), 4.00-4.11 (1H, m), |
| | 4.23 (2H, d, J = 6.5 Hz), 4.50 (2H, d, J = 6.3 Hz), 6.33 (1H, s), 6.47 (1H, d, J = 5.8 Hz), |
| | 6.75 (1H, s), 6.79 (1H, d, J = 1.3 Hz), 6.82 (2H, s), 6.95 (1H, dd, J = 5.3, 1.4 Hz), 6.97 |
| | (1H, d, J = 1.3 Hz), 7.36 (1H, t, J = 6.3 Hz), 7.62 (1H, d, J = 5.9 Hz), 8.05 (1H, d, J = 5.2 |
| | Hz), 9.05 (1H, s) |
| 4430 | 1.61-1.76 (1H, m), 2.03-2.16 (1H, m), 2.26-2.38 (1H, m), 2.39-2.48 (1H, m), |
| | 2.89 (1H, ddd, J = 16.2, 5.0, 1.5 Hz), 3.85 (1H, td, J = 12.0, 4.7 Hz), 4.05 (1H, ddd, J = |
| | 12.5, 5.5, 2.7 Hz), 4.15-4.27 (2H, m), 4.45 (2H, d, J = 5.9 Hz), 6.38 (1H, d, J = 7.7 |
| | Hz), 6.51 (2H, s), 6.75-6.79 (2H, m), 6.81 (1H, t, J = 6.1 Hz), 6.97 (1H, d, J = 1.3 Hz), |
| | 6.98 (1H, dd, J = 5.3, 1.3 Hz), 7.12 (1H, t, J = 8.0 Hz), 7.20 (1H, d, J = 6.1 Hz), 7.33 |
| | (1H, d, J = 8.3 Hz), 7.76 (1H, d, J = 6.1 Hz), 8.05 (1H, dd, J = 5.2, 0.6 Hz) |
| 4431 | 1.62-1.77 (1H, m), 2.01-2.14 (1H, m), 2.24-2.40 (1H, m), 2.40-2.47 (1H, m), |
| | 2.89 (1H, ddd, J = 16.2, 5.0, 1.5 Hz), 3.85 (1H, td, J = 12.0, 4.7 Hz), 4.05 (1H, ddd, J = |
| | 12.6, 5.5, 2.8 Hz), 4.16-4.26 (2H, m), 4.45 (2H, d, J = 5.9 Hz), 6.38 (1H, d, J = 7.7 |
| | Hz), 6.51 (2H, s), 6.77 (1H, s), 6.78 (1H, d, J = 1.2 Hz), 6.81 (1H, t, J = 6.1 Hz), 6.97 |
| | (1H, d, J = 1.3 Hz), 6.98 (1H, dd, J = 5.3, 1.4 Hz), 7.12 (1H, t, J = 8.0 Hz), 7.20 (1H, d, |
| | J = 5.8 Hz), 7.33 (1H, d, J = 8.3 Hz), 7.76 (1H, d, J = 6.1 Hz), 8.05 (1H, dd, J = 5.3, 0.7 Hz) |
| 4432 | 2.23 (2H, q, J = 6.5, 5.8 Hz), 2.92 (1H, dd, J = 16.8, 5.1 Hz), 3.15 (1H, dd, J = 16.8, 4.6 |
| | Hz), 3.90-4.09 (2H, m), 4.40 (2H, d, J = 6.1 Hz), 5.54 (1H, p, J = 4.9 Hz), 6.57 (2H, s), |
| | 6.64 (1H, d, J = 2.3 Hz), 6.76 (1H, d, J = 1.3 Hz), 6.81 (1H, d, J = 1.2 Hz), 6.93 (1H, dd, |
| | J = 9.1, 2.4 Hz), 6.98-7.04 (2H, m), 7.18 (1H, t, J = 6.0 Hz), 7.64 (1H, s), 7.93 (1H, d, |
| | J = 9.1 Hz), 8.12 (1H, d, J = 5.2 Hz) |
| 4433 | 4.44 (2H, d, J = 6.1 Hz), 5.37 (2H, s), 6.57 (2H, s), 6.66 (1H, d, J = 2.3 Hz), 6.92 (2H, |
| | dd, J = 7.1, 1.6 Hz), 6.95 (1H, dd, J = 9.0, 2.3 Hz), 7.04 (1H, dd, J = 5.3, 1.4 Hz), 7.22 |
| | (1H, t, J = 6.2 Hz), 7.54 (2H, d, J = 1.3 Hz), 7.65 (1H, s), 7.92 (1H, s), 7.94 (1H, d, J = |
| | 9.1 Hz), 8.12 (1H, d, J = 5.2 Hz), 8.51 (1H, dd, J = 7.0, 1.0 Hz) |
| 4434 | 1.52-1.66 (1H, m), 2.02 (1H, d, J = 13.4 Hz), 2.07-2.18 (1H, m), 2.29-2.40 (1H, |
| | m), 2.85 (1H, dd, J = 16.4, 5.0 Hz), 3.26 (2H, t), 3.78 (1H, td, J = 11.8, 4.7 Hz), 3.98- |
| | 4.08 (1H, m), 4.27 (2H, d, J = 6.0 Hz), 6.48 (2H, d, J = 2.7, 1.4 Hz), 6.57 (2H, s), 6.66 |
| | (1H, d, J = 2.3 Hz), 6.70 (1H, t, J = 5.8 Hz), 6.77 (1H, d, J = 1.2 Hz), 6.92 (1H, dd, J = |
| | 9.1, 2.4 Hz), 6.95 (1H, d, J = 1.3 Hz), 7.17 (1H, t, J = 6.0 Hz), 7.64 (1H, s), 7.86-7.96 |
| | (2H, m) |
| 4435 | 1.52-1.66 (1H, m), 2.02 (1H, d, J = 13.4 Hz), 2.06-2.23 (1H, m), 2.29-2.40 (1H, |
| | m), 2.80-2.90 (1H, m), 3.26 (2H, t), 3.78 (1H, td, J = 11.8, 4.7 Hz), 3.98-4.08 (1H, |
| | m), 4.27 (2H, d, J = 5.9 Hz), 6.48 (2H, dq, J = 2.7, 1.4 Hz), 6.57 (2H, s), 6.66 (1H, d, |
| | J = 2.3 Hz), 6.71 (1H, t, J = 5.8 Hz), 6.77 (1H, d, J = 1.2 Hz), 6.92 (1H, dd, J = 9.1, 2.3 |
| | Hz), 6.95 (1H, d, J = 1.3 Hz), 7.17 (1H, t, J = 6.0 Hz), 7.64 (1H, s), 7.86-7.96 (2H, m) |
| 4436 | 1.63-1.76 (1H, m), 2.06-2.17 (1H, m), 2.27-2.41 (1H, m), 2.43-2.49 (1H, m), |
| | 2.82-2.97 (1H, m), 3.86 (1H, td, J = 12.0, 4.7 Hz), 4.06 (1H, ddd, J = 12.6, 5.5, 2.7 |
| | Hz), 4.24 (2H, d, J = 6.5 Hz), 4.43 (2H, d, J = 6.1 Hz), 6.31 (2H, s), 6.51 (1H, d, J = 2.4 |
| | Hz), 6.79 (1H, d, J = 1.2 Hz), 6.81 (1H, dd, J = 1.4, 0.7 Hz), 6.93-7.03 (3H, m), 7.17 |
| | (1H, t, J = 6.2 Hz), 7.52 (1H, d, J = 2.9 Hz), 7.92 (1H, dd, J = 9.1, 2.5 Hz), 8.09 (1H, dd, |
| | J = 5.2, 0.7 Hz) |
| 4437 | 1.66-1.79 (1H, m), 2.09-2.16 (1H, m), 2.37 (1H, d, J = 10.2 Hz), 2.52-2.57 (1H, |
| | m), 2.95 (1H, dd, J = 16.4, 5.0 Hz), 3.90 (1H, td, J = 12.1, 4.7 Hz), 4.10 (1H, ddd, J = |
| | 12.8, 5.5, 2.6 Hz), 4.25 (2H, d, J = 6.5 Hz), 4.44 (2H, d, J = 6.1 Hz), 6.42 (2H, s), 6.52 |
| | (1H, d, J = 2.3 Hz), 6.80 (1H, s), 6.91-6.96 (1H, m), 6.96-7.03 (2H, m), 7.07-7.11 |
| | (1H, m), 7.22 (1H, t, J = 6.2 Hz), 7.53 (1H, d, J = 3.0 Hz), 7.94 (1H, dd, J = 9.1, 2.4 Hz), |
| | 8.09 (1H, d, J = 5.3 Hz) |
| 4438 | 1.65-1.78 (1H, m), 2.09-2.13 (1H, m), 2.32-2.37 (1H, m), 2.44-2.48 (1H, m), |
| | 2.90 (1H, dd, J = 16.1, 4.2 Hz), 3.82-3.89 (1H, m), 4.04-4.09 (1H, m), 4.24 (2H, d, |
| | J = 6.5 Hz), 4.42 (2H, d, J = 6.0 Hz), 6.59 (2H, s), 6.65 (1H, d, J = 2.2 Hz), 6.79 (1H, d, |
| | J = 1.0 Hz), 6.82 (1H, s), 6.95 (1H, dd, J = 9.1, 2.3 Hz), 6.98 (1H, d, J = 1.0 Hz), 7.00 |
| | (1H, dd, J = 5.3, 1.0 Hz), 7.23 (1H, t, J = 6.1 Hz), 7.65 (1H, s), 7.94 (1H, d, J = 9.1 Hz), |
| | 8.09 (1H, d, J = 5.1 Hz) |
| 4439 | 1.65-1.75 (1H, m), 2.09-2.15 (1H, m), 2.33-2.40 (1H, m), 2.44-2.48 (1H, m), |
| | 2.90 (1H, dd, J = 16.1, 4.2 Hz), 3.83-3.90 (1H, m), 4.04-4.09 (1H, m), 4.24 (2H, d, |
| | J = 6.5 Hz), 4.42 (2H, d, J = 6.0 Hz), 6.59 (2H, s), 6.65 (1H, d, J = 2.2 Hz), 6.79 (1H, d, |
| | J = 1.0 Hz), 6.82 (1H, s), 6.95 (1H, dd, J = 9.1, 2.3 Hz), 6.98 (1H, d, J = 1.1 Hz), 7.00 |
| | (1H, dd, J = 5.2, 1.1 Hz), 7.23 (1H, t, J = 6.1 Hz), 7.65 (1H, s), 7.94 (1H, d, J = 9.1 Hz), |
| | 8.09 (1H, d, J = 5.2 Hz) |
| 4440 | 2.12 (3H, s), 2.32-2.44 (1H, m), 2.54-2.60 (1H, m), 2.64-2.74 (1H, m), 3.26-3.29 |
| | (1H, m), 4.26 (2H, d, J = 5.9 Hz), 5.15-5.20 (1H, m), 6.49-6.54 (2H, m), 6.64 (2H, |
| | s), 6.67 (1H, d, J = 2.0 Hz), 6.73-6.82 (1H, m), 6.92 (1H, dd, J = 9.1, 2.1 Hz), 7.11- |
| | 7.18 (1H, m), 7.65 (1H, s), 7.92-7.99 (2H, m), 12.02 (1H, s). |
| 4441 | 1.58-1.74 (1H, m), 2.01 (3H, d, J = 1.0 Hz), 2.07 (1H, d, J = 12.3 Hz), 2.25-2.37 (1H, |
| | m), 2.37-2.47 (1H, m), 2.83 (1H, ddd, J = 16.1, 4.8, 1.4 Hz), 3.78 (1H, td, J = 11.8, |
| | 4.7 Hz), 3.96 (1H, ddd, J = 12.5, 5.6, 2.9 Hz), 4.23 (2H, d, J = 6.5 Hz), 4.42 (2H, d, J = |
| | 6.1 Hz), 6.57 (2H, s), 6.65 (2H, d, J = 2.3 Hz), 6.81 (1H, s), 6.95 (1H, dd, J = 9.0, 2.4 |
| | Hz), 7.00 (1H, dd, J = 5.3, 1.4 Hz), 7.21 (1H, t, J = 6.1 Hz), 7.65 (1H, s), 7.94 (1H, d, |
| | J = 9.1 Hz), 8.09 (1H, d, J = 5.2 Hz) |
| 4442 | 1.60-1.72 (1H, m), 2.01 (3H, s), 2.03-2.13 (1H, m), 2.24-2.36 (1H, m), 2.36-2.47 |
| | (1H, m), 2.83 (1H, dd, J = 16.1, 4.8 Hz), 3.78 (1H, td, J = 11.8, 4.7 Hz), 3.96 (1H, ddd, |
| | J = 12.5, 5.6, 2.9 Hz), 4.23 (2H, d, J = 6.5 Hz), 4.42 (2H, d, J = 6.1 Hz), 6.57 (2H, s), |
| | 6.62-6.68 (2H, m), 6.81 (1H, s), 6.95 (1H, dd, J = 9.1, 2.4 Hz), 7.00 (1H, d, J = 5.2 |
| | Hz), 7.21 (1H, t, J = 6.1 Hz), 7.65 (1H, s), 7.94 (1H, d, J = 9.0 Hz), 8.09 (1H, d, J = 5.2 |
| | Hz) impurity at 1.84 |
| 4443 | 1.66-1.76 (1H, m), 1.76-1.89 (1H, m), 1.98-2.12 (2H, m), 3.20-3.26 (1H, m), |
| | 3.88 (1H, ddd, J = 12.7, 9.2, 4.4 Hz), 3.99 (1H, dt, J = 12.4, 4.7 Hz), 4.28 (1H, dd, J = |
| | 10.5, 8.4 Hz), 4.41 (2H, d, J = 6.1 Hz), 4.77 (1H, dd, J = 10.6, 3.9 Hz), 6.58 (2H, s), |
| | 6.65 (1H, d, J = 2.3 Hz), 6.77 (1H, s), 6.83 (1H, d, J = 1.2 Hz), 6.94 (1H, dd, J = 9.0, 2.4 |
| | Hz), 7.00 (1H, dd, J = 5.2, 1.4 Hz), 7.01 (1H, d, J = 1.2 Hz), 7.21 (1H, t, J = 6.2 Hz), |
| | 7.65 (1H, s), 7.94 (1H, d, J = 9.1 Hz), 8.09 (1H, d, J = 5.2 Hz) |
| 4444 | 1.68 (1H, dtd, J = 13.3, 11.1, 5.7 Hz), 2.08 (3H, d, J = 1.1 Hz), 2.09-2.17 (1H, m), |
| | 2.20-2.35 (1H, m), 2.43 (1H, dd, J = 16.1, 10.7 Hz), 2.84 (1H, dd), 3.66 (1H, td, J = |
| | 11.8, 4.9 Hz), 3.85-3.95 (1H, m), 4.21 (2H, dd, J = 6.6, 2.0 Hz), 4.45 (2H, d, J = 6.0 |
| | Hz), 6.38 (1H, d, J = 7.7 Hz), 6.47-6.54 (3H, m), 6.77 (1H, s), 6.81 (1H, t, J = 6.1 Hz), |
| | 6.98 (1H, dd, J = 5.3, 1.4 Hz), 7.12 (1H, t, J = 8.0 Hz), 7.17-7.24 (1H, m), 7.33 (1H, d, |
| | J = 8.3 Hz), 7.77 (1H, d, J = 6.1 Hz), 8.05 (1H, d, J = 5.3 Hz) |
| 4445 | 1.59-1.76 (1H, m), 2.08 (3H, s), 2.09-2.18 (1H, m), 2.29 (1H, d, J = 11.3 Hz), 2.43 |
| | (1H, dd, J = 16.1, 10.8 Hz), 2.84 (1H, dd, J = 15.9, 4.8 Hz), 3.65 (1H, td, J = 11.7, 4.9 |
| | Hz), 3.90 (1H, ddd, J = 12.6, 5.7, 2.9 Hz), 4.15-4.27 (2H, m), 4.45 (2H, d, J = 5.9 Hz), |
| | 6.38 (1H, d, J = 7.7 Hz), 6.51 (3H, d, J = 9.1 Hz), 6.78 (1H, s), 6.81 (2H, t, J = 6.1 Hz), |
| | 6.98 (1H, dd, J = 5.2, 1.3 Hz), 7.12 (1H, t, J = 8.0 Hz), 7.20 (1H, d, J = 6.2 Hz), 7.33 |
| | (1H, d, J = 8.3 Hz), 7.77 (1H, d, J = 6.0 Hz), 8.05 (1H, d, J = 5.3 Hz) |
| 4446 | 1.61-1.74 (1H, m), 2.04 (3H, d, J = 1.1 Hz), 2.08 (1H, d, J = 13.5 Hz), 2.31 (1H, s), |
| | 2.42-2.47 (1H, m), 2.86 (1H, dd, J = 16.4, 5.0 Hz), 3.80 (1H, td, J = 11.8, 4.6 Hz), |
| | 3.94-4.03 (1H, m), 4.21 (2H, d, J = 6.5 Hz), 4.45 (2H, d, J = 5.9 Hz), 6.39 (1H, d, J = |
| | 7.7 Hz), 6.62 (2H, s), 6.74 (1H, d, J = 1.6 Hz), 6.76 (1H, t, J = 1.1 Hz), 6.84 (1H, t, J = |
| | 6.2 Hz), 6.99 (1H, dd, J = 5.3, 1.3 Hz), 7.13 (1H, t, J = 8.0 Hz), 7.18-7.23 (1H, m), |
| | 7.35 (1H, d, J = 8.3 Hz), 7.76 (1H, d, J = 6.1 Hz), 8.02-8.08 (1H, m) |
| 4447 | 1.59-1.71 (1H, m), 2.01 (3H, d, J = 0.9 Hz), 2.06 (1H, d, J = 12.9 Hz), 2.23-2.35 (1H, |
| | m), 2.41 (1H, dd, J = 16.1, 10.8 Hz), 2.75-2.85 (1H, m), 3.77 (1H, td, J = 11.9, 4.7 |
| | Hz), 3.95 (1H, ddd, J = 12.5, 5.6, 2.9 Hz), 4.13-4.26 (2H, m), 4.45 (2H, d, J = 5.9 Hz), |
| | 6.38 (1H, d, J = 7.7 Hz), 6.53 (2H, s), 6.64 (1H, d, J = 1.2 Hz), 6.77 (1H, s), 6.82 (1H, t, |
| | J = 6.0 Hz), 6.98 (1H, dd, J = 5.2, 1.4 Hz), 7.12 (1H, t, J = 8.0 Hz), 7.20 (1H, d, J = 6.1 |
| | Hz), 7.33 (1H, d, J = 8.3 Hz), 7.77 (1H, d, J = 6.0 Hz), 8.05 (1H, d, J = 5.2 Hz) |
| 4448 | 1.65-1.75 (1H, m), 2.14 (1H, d, J = 11.4 Hz), 2.29-2.42 (2H, m), 2.89 (1H, dd, J = |
| | 16.3, 4.5 Hz), 3.76-3.83 (1H, m), 4.06-4.11 (1H, m), 4.24 (2H, d, J = 6.6 Hz), 4.37 |
| | (2H, s), 4.42 (2H, d, J = 6.0 Hz), 5.00 (1H, br s), 6.57 (2H, s), 6.65 (1H, d, J = 2.2 Hz), |
| | 6.68 (1H, s), 6.83 (1H, s), 6.95 (1H, dd, J = 9.1, 2.3 Hz), 7.00 (1H, dd, J = 5.2, 1.0 Hz), |
| | 7.22 (1H, t, J = 6.1 Hz), 7.65 (1H, s), 7.94 (1H, d, J = 9.1 Hz), 8.09 (1H, d, J = 5.4 Hz) |
| 4449 | 1.61-1.75 (1H, m), 2.09 (1H, d, J = 13.5 Hz), 2.25-2.39 (1H, m), 2.40-2.47 (1H, |
| | m), 2.81-2.89 (1H, m), 3.82 (1H, td, J = 11.9, 4.7 Hz), 4.01 (1H, ddd, J = 12.5, 5.5, |
| | 2.9 Hz), 4.19-4.32 (4H, m), 4.42 (2H, d, J = 6.1 Hz), 4.69 (1H, s), 6.57 (2H, s), 6.65 |
| | (1H, d, J = 2.3 Hz), 6.80 (1H, d, J = 0.9 Hz), 6.82 (1H, d, J = 1.3 Hz), 6.95 (1H, dd, J = |
| | 9.0, 2.4 Hz), 7.00 (1H, dd, J = 5.3, 1.4 Hz), 7.21 (1H, t, J = 6.1 Hz), 7.65 (1H, s), 7.94 |
| | (1H, d, J = 9.1 Hz), 8.09 (1H, dd, J = 5.2, 0.7 Hz) |
| 4450 | 1.65-1.77 (1H, m), 1.77-1.91 (1H, m), 1.95-2.13 (2H, m), 3.22 (1H, td, J = 8.7, 4.7 |
| | Hz), 3.88 (1H, ddd, J = 12.6, 9.3, 4.4 Hz), 3.99 (1H, dt, J = 12.3, 4.6 Hz), 4.28 (1H, dd, |
| | J = 10.5, 8.4 Hz), 4.41 (2H, d, J = 6.1 Hz), 4.78 (1H, dd, J = 10.5, 4.0 Hz), 6.58 (2H, s), |
| | 6.65 (1H, d, J = 2.3 Hz), 6.77 (1H, s), 6.83 (1H, d, J = 1.2 Hz), 6.94 (1H, dd, J = 9.0, 2.4 |
| | Hz), 6.99 (1H, dd, J = 5.3, 1.3 Hz), 7.01 (1H, d, J = 1.3 Hz), 7.20 (1H, t, J = 6.1 Hz), |
| | 7.65 (1H, s), 7.94 (1H, d, J = 9.1 Hz), 8.09 (1H, d, J = 5.2 Hz) |
| 4451 | 1.66-1.77 (1H, m), 1.77-1.90 (1H, m), 1.97-2.12 (2H, m), 3.17-3.27 (1H, m), |
| | 3.88 (1H, ddd, J = 12.5, 9.2, 4.4 Hz), 3.99 (1H, dt, J = 11.9, 4.6 Hz), 4.27 (1H, dd, J = |
| | 10.6, 8.4 Hz), 4.41 (2H, d, J = 6.1 Hz), 4.78 (1H, dd, J = 10.6, 4.0 Hz), 6.57 (2H, s), |
| | 6.65 (1H, d, J = 2.3 Hz), 6.77 (1H, dd, J = 1.4, 0.7 Hz), 6.82 (1H, d, J = 1.2 Hz), 6.94 |
| | (1H, dd, J = 9.0, 2.4 Hz), 6.99 (2H, dd, J = 5.3, 1.3 Hz), 7.20 (1H, t, J = 6.1 Hz), 7.65 |
| | (1H, s), 7.94 (1H, d, J = 9.0 Hz), 8.09 (1H, dd, J = 5.3, 0.6 Hz) |
| 4452 | (CDCl₃, 400 MHz) 1.82 (1H, dtd, J = 13.5, 11.4, 5.6 Hz), 2.17-2.25 (1H, m), 2.38- |
| | 2.48 (1H, m), 2.59 (1H, dd, J = 16.6, 10.8 Hz), 2.96 (2H, dd, J = 9.0, 6.7 Hz), 3.04- |
| | 3.13 (1H, m), 3.28 (2H, dd, J = 8.9, 6.7 Hz), 3.95 (1H, td, J = 11.8, 4.7 Hz), 4.12 (1H, |
| | ddd, J = 12.4, 5.7, 2.8 Hz), 4.20-4.36 (2H, m), 6.38 (1H, s), 6.73 (1H, dd, J = 5.3, 1.4 |
| | Hz), 6.82 (1H, d, J = 1.4 Hz), 7.01 (1H, d, J = 1.4 Hz), 7.16 (1H, dd, J = 6.4, 0.9 Hz), |
| | 7.33-7.42 (2H, m), 7.81-7.85 (1H, m), 7.87 (1H, d, J = 6.4 Hz), 8.03 (1H, dd, J = 5.2, |
| | 0.7 Hz) |
| 4453 | 1.21-1.28 (1H, m), 1.65-1.76 (1H, m), 2.15 (1H, d, J = 11.7 Hz), 2.31-2.40 (1H, |
| | m), 2.52-2.60 (1H, m), 2.97 (1H, dd, J = 16.8, 4.6 Hz), 3.91-3.99 (1H, m), 4.24 (2H, |
| | d, J = 6.5 Hz), 4.42 (2H, d, J = 6.0 Hz), 4.48-4.54 (1H, m), 6.58 (2H, s), 6.65 (1H, d, |
| | J = 2.2 Hz), 6.83 (1H, s), 6.95 (1H, dd, J = 9.1, 2.2 Hz), 7.01 (1H, dd, J = 5.4, 0.8 Hz), |
| | 7.21 (1H, t, J = 6.1 Hz), 7.47 (1H, s), 7.65 (1H, s), 7.94 (1H, d, J = 9.1 Hz), 8.09 (1H, d, |
| | J = 5.3 Hz) |
| 4454 | 1.21-1.28 (m, 3H), 1.62-1.69 (m, 3H), 1.76-1.84 (m, 2H), 2.12 (s, 3H), 2.73 (d, J = |
| | 11.5 Hz, 2H), 4.05 (d, J = 6.1 Hz, 2H), 4.46 (d, J = 6.1 Hz, 2H), 5.89 (d, J = 7.6 Hz, 1H), |
| | 6.37 (t, J = 6.1 Hz, 1H), 6.67 (d, J = 8.3 Hz, 1H), 6.73 (s, 1H), 6.96 (dd, J = 5.3, 1.4 Hz, |
| | 1H), 7.05 (t, J = 8.0 Hz, 1H), 8.05 (d, J = 5.3 Hz, 1H) |
| 4455 | 1.19-1.30 (m, 2H), 1.60-1.69 (m, 3H), 1.76-1.85 (m, 2H), 2.13 (s, 3H), 2.52 (s, |
| | 3H), 2.71-2.77 (m, 2H), 4.06 (d, J = 6.1 Hz, 2H), 4.37 (d, J = 6.1 Hz, 2H), 6.74 (s, 1H), |
| | 6.87 (t, J = 6.2 Hz, 1H), 6.94 (dd, J = 5.3, 1.4 Hz, 1H), 7.24-7.29 (m, 1H), 8.06 (d, J = |
| | 5.2 Hz, 1H), 8.17 (d, J = 2.9 Hz, 1H), 8.37 (d, J = 1.8 Hz, 1H) |
| 4456 | 1.19-1.30 (m, 3H), 1.63-1.70 (m, 3H), 1.76-1.85 (m, 2H), 2.12-2.16 (m, 3H), |
| | 2.71-2.76 (m, 2H), 4.06 (d, J = 6.1 Hz, 2H), 4.31 (d, J = 6.0 Hz, 2H), 6.22 (d, J = 2.5 |
| | Hz, 1H), 6.62 (dd, J = 8.7, 2.5 Hz, 1H), 6.71 (s, 1H), 6.92 (dd, J = 5.3, 1.4 Hz, 1H), 7.20- |
| | 7.24 (m, 1H), 7.43 (d, J = 8.9 Hz, 1H), 7.68 (s, 1H), 8.07 (d, J = 5.3 Hz, 1H) |
| 4457 | (500 MHz, Methanol-d4) 1.38-1.48 (m, 2H), 1.77-1.87 (m, 3H), 2.07 (t, J = 11.8 |
| | Hz, 2H), 2.30 (s, 3H), 2.58 (s, 3H), 2.93 (d, J = 11.6 Hz, 2H), 4.12 (d, J = 6.0 Hz, 2H), |
| | 4.44 (s, 2H), 6.67-6.74 (m, 2H), 6.79 (s, 1H), 6.98 (dd, J = 5.4, 1.4 Hz, 1H), 7.73- |
| | 7.79 (m, 2H), 8.05 (dd, J = 5.3, 0.7 Hz, 1H) |
| 4458 | 1.19-1.30 (2H, m), 1.64-1.70 (3H, m), 1.81 (2H, td, J = 11.7, 2.3 Hz), 2.13 (3H, s), |
| | 2.71-2.77 (2H, m), 2.90 (6H, s), 4.06 (2H, d, J = 6.2 Hz), 4.43 (2H, d, J = 6.1 Hz), |
| | 6.65-6.72 (2H, m), 6.82 (1H, d, J = 9.6 Hz), 6.89 (1H, dd, J = 5.3, 1.4 Hz), 7.04 (1H, |
| | d, J = 9.6 Hz), 8.02 (1H, dd, J = 5.3, 0.7 Hz) |
| 4459 | 1.16-1.29 (2H, m), 1.57-1.70 (3H, m), 1.76-1.83 (2H, m), 2.12 (3H, s), 2.73 (2H, |
| | d, J = 10.7 Hz), 4.04 (2H, d, J = 6.1 Hz), 4.45 (2H, d, J = 6.3 Hz), 5.96 (1H, d, J = 5.5 |
| | Hz), 6.57 (1H, dd, J = 3.5, 1.9 Hz), 6.70 (1H, s), 6.94 (1H, dd, J = 5.3, 1.4 Hz), 7.03- |
| | 7.11 (1H, m), 7.20 (1H, t, J = 6.4 Hz), 7.73 (1H, d, J = 5.5 Hz), 8.04 (1H, d, J = 5.3 Hz), |
| | 11.17 (1H, s) |
| 4460 | 1.18-1.30 (2H, m), 1.59-1.70 (3H, m), 1.81 (2H, td, J = 11.7, 2.3 Hz), 2.13 (3H, s), |
| | 2.73 (2H, d, J = 11.7 Hz), 4.06 (2H, d, J = 6.1 Hz), 4.36 (2H, d, J = 6.3 Hz), 6.47 (1H, t, |
| | J = 6.3 Hz), 6.78 (1H, d, J = 1.4 Hz), 6.95-7.00 (2H, m), 7.95 (1H, d, J = 2.6 Hz), 8.06 |
| | (1H, d, J = 5.3 Hz), 8.15 (1H, s), 12.36 (1H, s) |
| 4461 | 1.16-1.29 (2H, m), 1.58-1.69 (3H, m), 1.76-1.83 (2H, m), 2.12 (3H, s), 2.73 (2H, |
| | d, J = 11.2 Hz), 4.05 (2H, d, J = 6.2 Hz), 4.47 (2H, d, J = 5.7 Hz), 6.42-6.48 (3H, m), |
| | 6.66-6.77 (2H, m), 6.97 (1H, dd, J = 5.3, 1.4 Hz), 7.22 (1H, t, J = 8.1 Hz), 7.32 (1H, |
| | dd, J = 8.1, 2.9 Hz), 7.64 (1H, d, J = 5.7 Hz), 8.05 (1H, d, J = 5.5 Hz) |
| 4462 | 1.16-1.36 (2H, m), 1.60-1.72 (3H, m), 1.79-1.95 (2H, m), 2.16 (3H, s), 2.77 (2H, |
| | d, J = 11.2 Hz), 3.92 (3H, s), 4.06 (2H, d, J = 6.1 Hz), 4.40 (2H, d, J = 6.1 Hz), 6.16 (1H, |
| | d, J = 1.8 Hz), 6.58 (1H, d, J = 1.9 Hz), 6.75 (1H, s), 6.97 (1H, dd, J = 5.3, 1.4 Hz), 7.11 |
| | (1H, t, J = 6.2 Hz), 7.26 (1H, dd, J = 5.9, 0.9 Hz), 8.07 (1H, dd, J = 5.3, 0.7 Hz), 8.14 |
| | (1H, d, J = 5.8 Hz), 9.03 (1H, s) |
| 4463 | 1.17-1.30 (2H, m), 1.56-1.70 (3H, m), 1.74-1.85 (2H, m), 2.12 (3H, s), 2.73 (2H, |
| | d, J = 11.1 Hz), 2.84 (3H, d, J = 4.5 Hz), 4.06 (2H, d, J = 6.0 Hz), 4.44 (2H, d, J = 6.1 |
| | Hz), 6.63 (1H, d, J = 2.2 Hz), 6.75 (1H, s), 6.96 (1H, d, J = 5.4 Hz), 7.20 (1H, d, J = 2.2 |
| | Hz), 7.28 (1H, t, J = 6.2 Hz), 7.39 (1H, d, J = 5.8 Hz), 8.07 (1H, d, J = 5.2 Hz), 8.19 (1H, |
| | d, J = 5.8 Hz), 8.50-8.59 (1H, m), 9.06 (1H, s) |
| 4464 | 1.16-1.29 (2H, m), 1.57-1.69 (3H, m), 1.78 (2H, td, J = 11.6, 2.2 Hz), 2.11 (3H, s), |
| | 2.60 (3H, s), 2.67-2.76 (2H, m), 4.05 (2H, d, J = 6.1 Hz), 4.39 (2H, d, J = 6.2 Hz), |
| | 6.41 (1H, d, J = 2.2 Hz), 6.74 (1H, s), 6.99-6.91 (2H, m), 7.08 (1H, t, J = 6.3 Hz), 7.31 |
| | (1H, dd, J = 5.8, 0.9 Hz), 8.06 (1H, d, J = 5.3 Hz), 8.17 (1H, d, J = 5.7 Hz), 9.01 (1H, s) |
| 4465 | 1.16-1.29 (2H, m), 1.57-1.71 (3H, m), 1.81 (2H, t, J = 11.2 Hz), 2.12 (3H, s), 2.31 |
| | (3H, s), 2.73 (2H, d, J = 11.2 Hz), 4.04 (2H, d, J = 6.1 Hz), 4.42 (2H, d, J = 6.3 Hz), 5.92 |
| | (1H, d, J = 5.5 Hz), 6.23 (1H, s), 6.68 (1H, s), 6.92 (1H, d, J = 5.3 Hz), 6.99 (1H, t, J = |
| | 6.4 Hz), 7.63 (1H, d, J = 5.5 Hz), 8.03 (1H, d, J = 5.3 Hz), 10.99 (1H, s) |
| 4466 | ¹H NMR (500 MHz, Methanol-d4) δ 1.26-1.47 (2H, m), 1.68-1.86 (3H, m), 1.92- |
| | 2.07 (2H, m), 2.26 (3H, s), 2.78-2.95 (2H, m), 4.08 (2H, d, J = 5.9 Hz), 4.59 (2H, s), |
| | 6.78 (1H, s), 6.97 (1H, dd, J = 5.3, 1.4 Hz), 7.11 (1H, t, J = 8.5 Hz), 7.66-7.72 (2H), |
| | 8.03 (1H, d, J = 5.3 Hz), 8.25 (1H, d, J = 6.1 Hz), 8.92 (1H, d, J = 1.7 Hz) |
| 4467 | 1.71 (1H, tq, J = 11.7, 5.5 Hz), 2.00-2.11 (1H, m), 2.85 (1H, ddd, J = 17.1, 11.2, 6.0 |
| | Hz), 2.98-3.09 (1H, m), 3.86 (1H, dd, J = 12.3, 10.4 Hz), 4.24 (1H, dd, J = 10.8, 7.2 |
| | Hz), 4.28-4.39 (2H, m), 4.46 (2H, d, J = 5.8 Hz), 6.43 (1H, d, J = 7.8 Hz), 6.77-6.81 |
| | (1H, m), 6.90 (3H, d, J = 7.7 Hz), 7.00 (1H, dd, J = 5.3, 1.4 Hz), 7.13-7.47 (5H, m), |
| | 7.75 (1H, d, J = 6.2 Hz), 8.07 (1H, dd, J = 5.2, 0.7 Hz) |
| 4468 | 0.83-0.90 (2H, m), 0.94-1.01 (2H, m), 1.96 (1H, tt, J = 8.3, 4.9 Hz), 3.63 (3H, s), |
| | 4.49 (2H, d, J = 5.8 Hz), 5.38 (2H, s), 6.56 (1H, d, J = 7.8 Hz), 6.80 (1H, s), 7.05 (1H, |
| | dd, J = 5.3, 1.4 Hz), 7.07 (1H, d, J = 5.9 Hz), 7.28 (1H, t, J = 8.1 Hz), 7.35 (1H, d, J = |
| | 6.7 Hz), 7.49 (1H, d, J = 8.3 Hz), 7.65 (2H, s), 7.71 (1H, d, J = 6.7 Hz), 8.13 (1H, dd, |
| | J = 5.3, 0.6 Hz). |
| 4469 | 1.17-1.28 (2H, m), 1.56-1.69 (3H, m), 1.75-1.84 (2H, m), 2.12 (3H, s), 2.69-2.76 |
| | (2H, m), 4.04 (2H, d, J = 6.1 Hz), 4.41 (2H, d, J = 5.9 Hz), 6.29 (1H, dd, J = 8.6, 4.0 |
| | Hz), 6.46 (2H, s), 6.65 (1H, t, J = 6.1 Hz), 6.70 (1H, s), 6.90-6.98 (2H, m), 7.21 (1H, |
| | dd, J = 6.2, 3.2 Hz), 7.84 (1H, d, J = 6.0 Hz), 8.03 (1H, d, J = 5.3 Hz) |
| 4470 | 1.13 (2H, qd, J = 12.1, 3.8 Hz), 1.38-1.50 (1H, m), 1.61-1.68 (2H, m), 1.72-1.81 |
| | (2H, m), 2.11 (3H, s), 2.68-2.75 (2H, m), 3.06 (2H, t, J = 6.3 Hz), 3.82 (3H, s), 4.16 |
| | (2H, d, J = 5.7 Hz), 5.88 (1H, t, J = 5.8 Hz), 6.36-6.42 (2H, m), 6.51 (1H, d, J = 8.5 |
| | Hz), 6.73 (1H, d, J = 8.5 Hz), 6.83 (1H, s), 7.04 (1H, d, J = 6.1 Hz), 7.35 (1H, dd, J = |
| | 8.5, 2.4 Hz), 7.71 (1H, d, J = 6.1 Hz), 7.95 (1H, d, J = 2.4 Hz) |
| 4471 | 4.10 (2H, t, J = 5.4 Hz), 4.24 (2H, t, J = 5.4 Hz), 4.34 (2H, d, J = 5.8 Hz), 4.97 (2H, s), |
| | 6.35 (2H, s), 6.47 (1H, d, J = 2.3 Hz), 6.52-6.55 (1H, m), 6.77-6.84 (2H, m), 6.88 |
| | (1H, dd, J = 9.1, 2.4 Hz), 7.13 (1H, s), 7.53 (1H, d, J = 5.8 Hz), 7.87 (1H, d, J = 9.0 Hz), |
| | 8.10 (1H, dd, J = 5.1, 0.7 Hz) |
| 4472 | 4.08 (2H, t, J = 5.4 Hz), 4.23 (2H, t, J = 5.4 Hz), 4.41 (2H, d, J = 5.9 Hz), 4.96 (2H, s), |
| | 6.43 (1H, d, J = 7.7 Hz), 6.50 (2H, s), 6.73-6.78 (2H, m), 7.07-7.15 (2H, m), 7.20 |
| | (1H, d, J = 6.1 Hz), 7.32 (1H, d, J = 8.3 Hz), 7.76 (1H, d, J = 6.1 Hz), 8.07 (1H, d, J = |
| | 5.1 Hz) |
| 5434 | 1.67-1.73 (4H, m), 2.40-2.47 (4H, m), 3.63 (2H, s), 4.50 (2H, d, J = 6.1 Hz), 6.31 |
| | (2H, s), 6.50 (1H, d, J = 2.3 Hz), 6.54 (1H, d, J = 5.9 Hz), 6.90-6.94 (2H, m), 7.32- |
| | 7.37 (2H, m), 7.40-7.44 (1H, m), 7.53 (1H, d, J = 5.9 Hz), 7.86-7.92 (2H, m), 7.96 |
| | (1H, s), 7.98-8.00 (1H, m), 8.58 (1H, d, J = 5.0 Hz) |
| 5435 | 2.17 (3H, s), 2.55-2.65 (1H, m), 2.79-2.89 (3H, m), 3.21-3.26 (2H, m), 3.55-3.62 |
| | (2H, m), 3.92-3.98 (2H, m), 4.28 (2H, d, J = 6.0 Hz), 6.32 (2H, s), 6.34-6.37 (1H, |
| | m), 6.43 (1H, d, J = 2.4 Hz), 6.53 (1H, d, J = 5.8 Hz), 6.62 (1H, d, J = 5.3, 1.4 Hz), 6.77 |
| | (1H, t, J = 5.9 Hz), 6.87 (1H, dd, J = 9.0, 2.4 Hz), 7.54 (1H, d, J = 5.8 Hz), 7.86 (1H, d, |
| | J = 9.1 Hz), 7.97 (1H, d, J = 5.2 Hz) |
| 5436 | 3.98-4.03 (2H, m), 4.03-4.07 (2H, m), 4.41 (2H, d, J = 5.9 Hz), 4.65 (2H, s), 6.44 |
| | (1H, d, J = 7.7 Hz), 6.51 (2H, s), 6.72 (1H, dd, J = 5.1, 1.2 Hz), 6.76 (1H, t, J = 6.1 Hz), |
| | 6.87 (1H, d, J = 1.2 Hz), 7.04 (1H, s), 7.09 (1H, d, J = 1.2 Hz), 7.12 (1H, t, J = 8.0 Hz), |
| | 7.21 (1H, d, J = 6.1 Hz), 7.32 (1H, d, J = 8.3 Hz), 7.76 (1H, d, J = 6.1 Hz), 8.05 (1H, d, |
| | J = 5.1 Hz) |
| 5437 | 3.97-4.04 (2H, m), 4.05-4.11 (2H, m), 4.37 (2H, d, J = 6.0 Hz), 4.68 (2H, s), 6.59 |
| | (2H, s), 6.69 (1H, d, J = 2.4 Hz), 6.74 (1H, dd, J = 5.1, 1.2 Hz), 6.88 (1H, d, J = 1.3 Hz), |
| | 6.96 (1H, dd, J = 9.1, 2.4 Hz), 7.06 (1H, s), 7.09 (1H, d, J = 1.2 Hz), 7.18 (1H, t, J = 6.1 |
| | Hz), 7.65 (1H, s), 7.94 (1H, d, J = 9.1 Hz), 8.09 (1H, d, J = 5.0 Hz) |
| 5441 | 0.64-0.70 (1H, m), 0.88-0.94 (6H, m), 1.07-1.11 (1H, m), 1.34-1.69 (5H, m), |
| | 1.84-1.99 (2H, m), 2.54-2.71 (3H, m), 3.00-3.24 (5H, m), 3.38-3.45 (2H, m), |
| | 4.49 (2H, d, J = 4.9 Hz), 6.43 (1H, d, J = 7.7 Hz), 6.48 (2H, s), 6.81 (1H, s), 7.08-7.13 |
| | (2H, m), 7.20 (1H, d, J = 6.2 Hz), 7.28-7.34 (2H, m), 7.35 (1H, t, J = 7.6 Hz), 7.42 |
| | (1H, d, J = 7.6 Hz), 7.75 (1H, d, J = 6.0 Hz) |
| 5442 | 3.80 (4H, t, J = 7.3 Hz), 3.90 (1H, t, J = 5.3 Hz), 3.99 (1H, t, J = 5.5 Hz), 4.37 (2H, t, J = |
| | 5.9 Hz), 4.68 (2H, d, J = 49.5 Hz), 6.54 (2H, s), 6.65-6.74 (2H, m), 6.94 (1H, dd, J = |
| | 9.0, 2.4 Hz), 7.09-7.18 (2H, m), 7.27 (3H, dd, J = 7.9, 4.6 Hz), 7.53 (1H, d, J = 26.3 |
| | Hz), 7.63 (1H, d, J = 2.0 Hz), 7.90 (1H, d, J = 9.1 Hz), 8.23 (1H, s) |
| 5443 | 1.61-1.75 (4H, m), 2.41-2.47 (4H, m), 3.64 (2H, s), 4.55 (2H, d, J = 6.1 Hz), 6.65 |
| | (1H, d, J = 2.3 Hz), 7.18 (1H, dd, J = 8.9, 2.2 Hz), 7.26 (1H, t, J = 6.0 Hz), 7.33-7.39 |
| | (3H, m), 7.41-7.47 (1H, m), 7.79 (1H, d, J = 8.9 Hz), 7.90-7.93 (1H, m), 7.97 (1H, |
| | s), 7.99-8.01 (1H, m), 8.16 (1H, d, J = 5.8 Hz), 8.60 (1H, d, J = 5.0 Hz), 8.88 (1H, s) |
| 8458 | 2.12-2.23 (1H, m), 2.38-2.47 (1H, m), 3.18 (1H, dd, J = 16.4, 9.7 Hz), 3.38 (1H, dd, |
| | J = 16.8, 5.3 Hz), 3.55-3.63 (1H, m), 4.12-4.22 (2H, m), 4.47 (2H, d, J = 5.6 Hz), |
| | 6.45-6.59 (3H, m), 6.72 (1H, d, J = 7.7 Hz), 7.02 (1H, s), 7.13 (1H, d, J = 5.8 Hz), |
| | 7.21 (1H, t, J = 8.0 Hz), 7.38 (1H, d, J = 8.3 Hz), 7.73 (1H, d, J = 6.1 Hz) |
| 8459 | 3.63 (2H, t, J = 5.5 Hz), 4.24 (2H, t, J = 5.5 Hz), 4.50-4.57 (4H, m), 6.44 (1H, d, J = |
| | 1.8 Hz), 6.50 (2H, s), 6.62 (1H, d, J = 7.7 Hz), 6.73 (1H, t, J = 5.9 Hz), 7.11-7.16 (2H, |
| | m), 7.16 (1H, t, J = 8.0 Hz), 7.35 (1H, d, J =8.3 Hz), 7.74 (1H, d, J = 6.0 Hz) |
| 9001 | 3.80 (t, J = 5.5 Hz, 2H), 4.28 (t, J = 5.4 Hz, 2H), 4.44 (d, J = 5.8 Hz, 2H), 4.71 (s, 2H), |
| | 6.45 (d, J = 7.6 Hz, 1H), 6.50 (s, 2H), 6.76 (t, J = 6.0 Hz, 1H), 7.11 (t, J = 8.0 Hz, 1H), |
| | 7.17 (d, J = 6.1 Hz, 1H), 7.24 (d, J = 8.6 Hz, 1H), 7.31 (d, J = 8.3 Hz, 1H), 7.48 (dd, J = |
| | 8.7, 3.0 Hz, 1H), 7.76 (d, J = 6.1 Hz, 1H), 8.43 (d, J = 2.9 Hz, 1H) |
| 9002 | 4.13 (2H, t, J = 5.4 Hz), 4.26 (2H, t, J = 5.4 Hz), 4.46 (2H, d, J = 5.7 Hz), 5.02 (2H, s), |
| | 6.50 (2H, s), 6.55 (1H, d, J = 7.7 Hz), 6.69 (1H, t, J = 6.0 Hz), 7.10-7.19 (2H, m), 7.32 |
| | (1H, d, J = 8.3 Hz), 7.74 (1H, d, J = 6.1 Hz), 8.17 (1H, d, J = 1.4 Hz), 8.51 (1H, d, J = |
| | 1.5 Hz) |
| 9003 | 4.19-4.27 (4H, m), 4.32 (2H, d, J = 5.6 Hz), 5.11 (2H, s), 6.49 (2H, s), 6.56 (1H, t, J = |
| | 5.9 Hz), 6.61 (1H, d, J = 7.7 Hz), 7.14 (1H, d, J = 5.9 Hz), 7.17 (1H, t, J = 8.0 Hz), 7.34 |
| | (1H, d, J = 8.3 Hz), 7.73 (1H, d, J = 6.1 Hz), 8.52 (2H, s) |
| 9004 | 4.16 (2H, t, J = 5.4 Hz), 4.28 (2H, t, J = 5.4 Hz), 4.58 (2H, d, J = 5.9 Hz), 5.07 (2H, s), |
| | 6.51 (2H, s), 6.55 (1H, d, J = 7.7 Hz), 6.82 (1H, t, J = 6.1 Hz), 7.09-7.18 (2H, m), 7.32 |
| | (1H, d, J = 8.3 Hz), 7.42-7.51 (2H, m), 7.75 (1H, d, J = 6.1 Hz) |
| 9005 | 2.37 (3H, s), 3.84 (2H, t, J = 5.5 Hz), 4.29 (2H, t, J = 5.4 Hz), 4.40 (2H, d, J = 4.6 Hz), |
| | 4.74 (2H, s), 6.45-6.60 (3H, m), 6.77 (1H, d, J = 7.7 Hz), 7.05 (1H, d, J = 6.1 Hz), |
| | 7.21 (1H, t, J = 8.0 Hz), 7.34 (1H, d, J = 8.3 Hz), 7.43 (1H, d, J = 2.7 Hz), 7.76 (1H, d, |
| | J = 6.1 Hz), 8.30 (1H, d, J = 2.8 Hz) |
| 9006 | 2.30 (s, 3H), 2.42 (s, 3H), 4.11 (t, J = 5.4 Hz, 2H), 4.19 (d, J = 4.3 Hz, 2H), 4.25 (t, J = |
| | 5.4 Hz, 2H), 5.00 (s, 2H), 5.61 (t, J = 4.4 Hz, 1H), 6.48 (s, 2H), 6.75 (d, J = 7.7 Hz, 1H), |
| | 6.84 (s, 1H), 7.16 (d, J = 6.1 Hz, 1H), 7.27 (t, J = 8.0 Hz, 1H), 7.38 (d, J = 8.3 Hz, 1H), |
| | 7.65 (d, J = 6.1 Hz, 1H) |
| 9007 | 1.47 (3H, d, J = 6.8 Hz), 2.37 (3H, s), 3.60 (1H, ddd, J = 14.4, 9.8, 4.8 Hz), 4.04-4.12 |
| | (1H, m), 4.17-4.28 (2H, m), 4.39 (2H, d, J = 4.7 Hz), 5.48 (1H, q, J = 6.7 Hz), 6.48- |
| | 6.57 (3H, m), 6.77 (1H, d, J = 7.7 Hz), 7.04 (1H, d, J = 6.1 Hz), 7.21 (1H, t, J = 8.0 Hz), |
| | 7.34 (1H, d, J = 8.3 Hz), 7.41 (1H, d, J = 2.8 Hz), 7.76 (1H, d, J = 6.1 Hz), 8.28 (1H, d, |
| | J = 2.8 Hz) |
| 9008 | 1.50 (3H, d, J = 6.7 Hz), 3.62 (1H, dt, J = 14.6, 7.7 Hz), 4.11-4.24 (3H, m), 4.46 (2H, |
| | d, J = 5.7 Hz), 5.56 (1H, q, J = 6.7 Hz), 6.42-6.57 (3H, m), 6.71 (1H, d, J = 7.7 Hz), |
| | 7.12 (1H, d, J = 6.1 Hz), 7.17 (1H, t, J = 8.0 Hz), 7.32 (1H, d, J = 8.3 Hz), 7.47 (1H, dd, |
| | J = 13.3, 2.5 Hz), 7.73 (1H, d, J = 6.1 Hz), 8.28 (1H, s) |
| 10001 | 1.78-1.97 (2H, m), 2.04-2.13 (1H, m), 2.33-2.41 (1H, m), 2.87-2.95 (1H, m), |
| | 3.92-4.00 (2H, m), 4.01-4.08 (1H, m), 4.14 (2H, t, J = 5.5 Hz), 4.69 (1H, dd, J = 9.2, |
| | 6.5 Hz), 4.77-4.87 (2H, m), 6.58 (2H, s), 6.91 (1H, d, J = 8.5 Hz), 7.05-7.12 (1H, m), |
| | 7.19 (1H, t, J = 7.9 Hz), 7.22 (1H, d, J = 6.2 Hz), 7.37 (1H, t, J = 51.9 Hz), 7.60 (1H, dd, |
| | J = 8.8, 2.4 Hz), 7.66 (1H, d, J = 8.3 Hz), 7.78 (1H, d, J = 6.0 Hz), 8.18 (1H, d, J = 2.3 Hz) |
| 10002 | 1.75-1.97 (2H, m), 2.04-2.14 (1H, m), 2.32-2.41 (1H, m), 2.86-2.95 (1H, m), |
| | 3.91-4.08 (3H, m), 4.12-4.22 (2H, m), 4.70 (1H, t, J = 7.9 Hz), 4.80-4.91 (2H, m), |
| | 6.59 (2H, s), 6.92 (1H, d, J = 8.8 Hz), 7.09 (1H, d, J = 7.6 Hz), 7.15-7.26 (2H, m), |
| | 7.61 (1H, d, J = 8.7 Hz), 7.66 (1H, d, J = 8.2 Hz), 7.78 (1H, dd, J = 6.0, 1.7 Hz), 8.18 |
| | (1H, s) |
| 10003 | 1.75-1.97 (2H, m), 2.04-2.14 (1H, m), 2.32-2.41 (1H, m), 2.86-2.95 (1H, m), |
| | 3.91-4.08 (3H, m), 4.12-4.22 (2H, m), 4.70 (1H, t, J = 7.9 Hz), 4.80-4.91 (2H, m), |
| | 6.59 (2H, s), 6.92 (1H, d, J = 8.8 Hz), 7.09 (1H, d, J = 7.6 Hz), 7.15-7.26 (2H, m), |
| | 7.61 (1H, d, J = 8.7 Hz), 7.66 (1H, d, J = 8.2 Hz), 7.78 (1H, dd, J = 6.0, 1.7 Hz), 8.18 |
| | (1H, s) |
| 10004 | 1.75-1.97 (2H, m), 2.04-2.14 (1H, m), 2.32-2.41 (1H, m), 2.86-2.95 (1H, m), |
| | 3.91-4.08 (3H, m), 4.12-4.22 (2H, m), 4.70 (1H, t, J = 7.9 Hz), 4.80-4.91 (2H, m), |
| | 6.59 (2H, s), 6.92 (1H, d, J = 8.8 Hz), 7.09 (1H, d, J = 7.6 Hz), 7.15-7.26 (2H, m), |
| | 7.61 (1H, d, J = 8.7 Hz), 7.66 (1H, d, J = 8.2 Hz), 7.78 (1H, dd, J = 6.0, 1.7 Hz), 8.18 |
| | (1H, s) |

Biological Methods

Determination of FXIIa Inhibition

In vitro inhibition of Factor XIIa was determined using an IC50 assay based on standard literature methods (see e.g Baeriswyl et al., ACS Chem. Biol., 2015, 10 (8) 1861; Bouckaert et al., European Journal of Medicinal Chemistry 110 (2016) 181). Human Factor XIIa (Enzyme Research Laboratories) was incubated at 25° C. with the fluorogenic substrate H-DPro-Phe-Arg-AFC (Peptide Protein Science) and various concentrations of the test compound. Protease activity was measured by monitoring the accumulation of liberated fluorescence from the substrate over 5 min at 25° C. The linear rate of fluorescence increase per minute was expressed as percentage (%) activity. The Km for the cleavage of the substrate by FXIIa was determined by standard transformation of the Michaelis-Menten equation. The compound inhibitor assays were performed at substrate Km concentration. IC₅₀ values were calculated as the concentration of inhibitor giving 50% inhibition (IC₅₀) of the uninhibited enzyme activity (100%). Data acquired from this assay are shown in Table 13 below using the following scale:

| Category | IC₅₀ (nM) |
|---|---|
| A | 10-100 |
| B | 100-300 |
| C | 300-1,000 |
| D | 1,000-3,000 |
| E | 3,000-10,000 |
| F | 10,000-40,000 |
| G | 40,000-100,000 |

For the test compounds that did not achieve 50% inhibition the following scale is used:

| Category | |
|---|---|
| H1 | >20% inhibition @ 40 μM |
| H2 | >20% inhibition @ 100 μM |
| H3 | >20% inhibition @ 400 μM |

TABLE 13
Human FXIIa data, molecular weight and LCMS data

| | Human FXIIa | | |
|---|---|---|---|
| Example Number | IC50 (nM) | Molecular weight | LCMS Mass Ion |
| 1001 | C | 363.5 | 364.1 |
| 1002 | B | 362.5 | 363.1 |
| 1003 | E | 362.5 | 363.2 |
| 1004 | F | 370.9 | 371.1 |
| 1005 | B | 377.5 | 378.4 |
| 1006 | B | 400.5 | 401.5 |
| 1007 | C | 361.5 | 362.5 |
| 1008 | E | 362.5 | 363.2 |
| 1009 | B | 377.5 | 378.2 |
| 1010 | B | 391.5 | 392.2 |
| 1011 | E | 377.5 | 378.2 |
| 1012 | B | 405.5 | 406.2 |
| 1013 | B | 378.5 | 379.2 |
| 1014 | E | 357.4 | 358.4 |
| 1015 | C | 391.5 | 392.5 |
| 1016 | B | 363.5 | 364.2 |
| 1017 | C | 435.6 | 436.2 |
| 1018 | C | 376.5 | 377.2 |
| 1019 | E | 376.5 | 377.2 |
| 1020 | D | 385.9 | 386.2 |
| 1021 | F | 378.5 | 379.2 |
| 1022 | F | 378.5 | 379.2 |
| 1023 | B | 400.5 | 401.2 |
| 1024 | B | 400.5 | 401.2 |
| 1025 | C | 388.5 | 389.2 |
| 1026 | F | 392.5 | 393.2 |
| 1027 | B | 407.5 | 408.5 |
| 1028 | D | 468.5 | 469.4 |
| 1029 | B | 400.5 | 401.4 |
| 1030 | C | 400.5 | 401.5 |
| 1031 | D | 374.4 | 375.3 |
| 1032 | C | 391.5 | 392.5 |
| 1033 | A | 376.5 | 377.2 |
| 1034 | F | 366.4 | 367.1 |
| 1035 | E | 389.5 | 390.1 |
| 1036 | C | 419.5 | 420.2 |
| 1037 | C | 419.5 | 420.2 |
| 1038 | E | 422.9 | 423.1 |
| 1039 | F | 391.5 | 392.2 |
| 1040 | F | 425.9 | 426.2 |
| 1041 | C | 434.9 | 435.1 |
| 1042 | B | 401.5 | 402.2 |
| 1043 | C | 434.9 | 435.2 |
| 1044 | B | 433.9 | 434.1 |
| 1049 | C | 386.5 | 387.2 |
| 1050 | D | 420.9 | 421.1 |
| 1052 | C | 435.9 | 436.2 |
| 1096 | E | 326.4 | 327.3 |
| 1102 | E | 375.5 | 376.4 |
| 1103 | F | 391.2 | 392.2 |
| 1116 | H2 | 410.9 | 411.4 |
| 1118 | F | 419.9 | 420.2 |
| 1119 | F | 385.5 | 386.2 |
| 1128 | C | 371.4 | 372.2 |
| 1129 | E | 405.9 | 406.2 |
| 1130 | E | 377.5 | 378.2 |
| 1131 | D | 377.5 | 378.3 |
| 1133 | E | 401.5 | 402.4 |
| 1135 | E | 429.5 | 430.1 |
| 1137 | D | 401.5 | 402.4 |
| 1140 | E | 401.5 | 402.2 |
| 1150 | C | 385.5 | 386.2 |
| 1156 | F | 371.4 | 372.2 |
| 1157 | H1 | 405.9 | 406.2 |
| 1163 | E | 429.5 | 430.2 |
| 1167 | E | 429.5 | 430.3 |
| 1175 | C | 375.5 | 376.5 |
| 1176 | E | 411.9 | 412.2 |
| 1177 | H2 | 391.5 | 392.2 |
| 1178 | H2 | 390.5 | 391.3 |
| 1180 | H2 | 390.5 | 391.3 |
| 1181 | F | 409.4 | 410.2 |
| 1182 | F | 443.9 | 444.1 |
| 1183 | H2 | 361.5 | 362.5 |
| 1184 | F | 361.5 | 362.4 |
| 1185 | F | 429.9 | 430.1 |
| 1186 | H2 | 355.5 | 356.5 |
| 1187 | H2 | 351.5 | 352.2 |
| 1188 | G | 395.9 | 396.3 |
| 1189 | G | 363.5 | 364.5 |
| 1190 | H2 | 350.5 | 351.4 |
| 1191 | H2 | 368.5 | 369.3 |
| 1192 | H2 | 364.5 | 365.3 |
| 1193 | B | 410.9 | 411.5 |
| 1194 | E | 394.5 | 395.5 |
| 1195 | B | 394.5 | 395.5 |
| 1196 | D | 377.5 | 378.3 |
| 1197 | D | 350.5 | 351.5 |
| 1198 | G | 444.6 | 445.5 |
| 1199 | H2 | 366.5 | 367.6 |
| 1200 | H2 | 397.5 | 398.3 |
| 1201 | G | 377.5 | 378.3 |
| 1202 | B | 364.5 | 365.3 |
| 1203 | H2 | 405.5 | 406.3 |
| 1204 | H2 | 427.9 | 428.3 |
| 1205 | F | 364.5 | 365.3 |
| 1206 | G | 368.5 | 369.3 |
| 1207 | G | 379.5 | 380.5 |
| 1208 | E | 395.9 | 396.4 |
| 1209 | E | 395.9 | 396.5 |
| 1210 | H2 | 418.5 | 419.3 |
| 1211 | F | 391.5 | 392.3 |
| 1212 | H2 | 375.5 | 376.3 |
| 1213 | E | 350.5 | 351.4 |
| 1214 | H2 | 379.5 | 380.4 |
| 1215 | H2 | 363.5 | 364.4 |
| 1216 | H2 | 378.5 | 379.4 |
| 1217 | G | 391.5 | 392.3 |
| 1218 | H2 | 351.5 | 352.3 |
| 1219 | E | 340.5 | 341.3 |
| 1220 | H2 | 441.0 | 441.3/443.3 |
| 1221 | H2 | 375.5 | 376.5 |
| 1222 | H2 | 350.5 | 351.3 |
| 1223 | G | 350.5 | 351.3 |
| 1224 | E | 379.5 | 380.3 |
| 1225 | H2 | 364.5 | 365.3 |
| 1226 | E | 364.5 | 365.3 |
| 1227 | H2 | 351.5 | 352.3 |
| 1228 | H2 | 363.5 | 364.3 |
| 1229 | H2 | 351.5 | 352.3 |
| 1230 | H2 | 349.5 | 350.3 |
| 1231 | H2 | 392.5 | 393.4 |
| 1232 | C | 364.5 | 365.3 |
| 1233 | H2 | 380.5 | 381.7 |
| 1234 | H2 | 364.5 | 365.3 |
| 1235 | H2 | 364.5 | 365.3 |
| 1236 | H2 | 366.5 | 367.1 |
| 1237 | F | 417.5 | 418.5 |
| 1238 | H2 | 431.5 | 432.7 |
| 1239 | F | 378.5 | 379.3 |
| 1240 | H2 | 363.5 | 364.3 |
| 1241 | F | 383.9 | 384.3 |
| 1242 | H2 | 379.5 | 380.3 |
| 1243 | A | 384.9 | 385.3 |
| 1244 | H2 | 364.5 | 365.3 |
| 1245 | E | 394.5 | 395.4 |
| 1246 | E | 364.5 | 365.3 |
| 1247 | H2 | 408.5 | 409.3 |
| 1248 | F | 408.5 | 409.3 |
| 1249 | C | 365.5 | 366.6 |
| 1250 | D | 402.45 | 403.4 |
| 1251 | A | 384.9 | 385.3 |
| 1252 | F | 393.5 | 394.4 |
| 1253 | H2 | 394.5 | 395.4 |
| 1254 | F | 365.5 | 366.4 |
| 1255 | E | 398.9 | 399.4 |
| 1256 | F | 374.5 | 375.2 |
| 1257 | H2 | 380.5 | 381.5 |
| 1258 | F | 311.4 | 312.4 |
| 1259 | H2 | 406.5 | 407.5 |
| 1260 | H1 | 466.6 | 467.5 |
| 1261 | H1 | 375.5 | 376.4 |
| 1262 | D | 451.9 | 453.5 |
| 1263 | F | 394.5 | 395.2 |
| 1264 | H1 | 393.5 | 394.6 |
| 1265 | E | 383.4 | 384.2 |
| 1266 | F | 417.9 | 418.2 |
| 1267 | E | 421.9 | 422.2 |
| 1268 | E | 401.5 | 402.3 |
| 1269 | F | 401.5 | 402.2 |
| 1270 | F | 447.9 | 448.1 |
| 1271 | C | 385.5 | 386.2 |
| 1272 | D | 419.9 | 420.1 |
| 1273 | E | 435.9 | 436.1 |
| 1274 | C | 422.4 | 423.3 |
| 1275 | C | 440.4 | 441.3 |
| 1276 | D | 372.4 | 373.3 |
| 1277 | C | 430.8 | 431.2/433.2 |
| 1278 | D | 410.4 | 411.3 |
| 1279 | B | 386.5 | 387.3 |
| 1280 | C | 448.8 | 449.3/451.3 |
| 1281 | F | 386.4 | 387.4 |
| 1282 | A | 454.5 | 455.2 |
| 1283 | E | 425.4 | 426.4 |
| 1284 | C | 454.5 | 455.2 |
| 1285 | C | 454.5 | 455.4 |
| 1286 | C | 385.5 | 386.2 |
| 1287 | D | 454.5 | 455.2 |
| 1288 | H3 | 414.4 | 415.4 |
| 1289 | H1 | 448.8 | 449.4 |
| 1290 | H3 | 433.4 | 434.4 |
| 1291 | E | 375.4 | 376.3 |
| 1292 | H1 | 473.8 | 474.3 |
| 1293 | H1 | 462.9 | 463.7 |
| 1294 | F | 454.5 | 455.2 |
| 1295 | A | 454.5 | 455.2 |
| 1296 | D | 454.5 | 455.2 |
| 1297 | D | 454.5 | 455.1 |
| 1298 | D | 508.4 | 509.2 |
| 1299 | A | 474.9 | 475.4/477.4 |
| 1300 | B | 439.4 | 440.4 |
| 1301 | B | 508.4 | 509.7 |
| 1302 | F | 454.5 | 455.4 |
| 1303 | A | 468.5 | 469.5 |
| 1304 | E | 468.5 | 469.2 |
| 1305 | A | 468.5 | 469.2 |
| 1306 | H1 | 508.4 | 509.6 |
| 1307 | C | 480.5 | 481.3 |
| 1308 | D | 482.5 | 483.4 |
| 1309 | A | 468.5 | 469.3 |
| 1310 | D | 400.5 | 401.4 |
| 1311 | A | 454.5 | 455.4 |
| 1312 | E | 387.4 | 388.2 |
| 1313 | B | 462.9 | 463.3 |
| 1314 | A | 476.9 | 477.2 |
| 1315 | F | 476.9 | 477.3 |
| 1316 | A | 476.9 | 477.3 |
| 1317 | H3 | 441.4 | 442.3 |
| 1318 | F | 441.4 | 442.3 |
| 1319 | A | 498.5 | 499.4 |
| 1320 | F | 482.5 | 483.4 |
| 1321 | B | 482.5 | 483.4 |
| 1322 | F | 428.4 | 429.4 |
| 1323 | F | 404.4 | 405.4 |
| 1324 | H1 | 387.5 | 388.4 |
| 1325 | H1 | 395.9 | 396.3 |
| 1326 | H1 | 376.5 | 377.5 |
| 1327 | H1 | 363.5 | 364.2 |
| 1328 | H1 | 420.5 | 421.3 |
| 1329 | H3 | 406.5 | 407.2 |
| 1330 | H3 | 405.5 | 406.2 |
| 1331 | B | 482.5 | 483.4 |
| 1332 | E | 454.5 | 455.9 |
| 1333 | B | 454.5 | 455.4 |
| 1334 | C | 488.9 | 489.4 |
| 1335 | F | 470.5 | 471.4 |
| 1336 | C | 468.5 | 469.4 |
| 1337 | C | 490.5 | 491.3 |
| 1338 | H1 | 438.4 | 439.4 |
| 1339 | F | 430.4 | 431.3 |
| 1341 | D | 442.4 | 443.0 |
| 1342 | A | 468.5 | 469.2 |
| 1343 | E | 498.5 | 499.2 |
| 1344 | A | 498.5 | 499.2 |
| 1345 | A | 468.5 | 469.2 |
| 1346 | D | 468.5 | 469.2 |
| 2177 | B | 374.5 | 375.5 |
| 2178 | A | 374.5 | 375.2 |
| 2179 | D | 389.5 | 390.7 |
| 2180 | C | 375.5 | 376.5 |
| 2181 | C | 393.5 | 394.5 |
| 2182 | C | 393.5 | 394.2 |
| 2183 | C | 380.5 | 381.2 |
| 2184 | E | 428.0 | 428.1 |
| 2185 | B | 376.5 | 377.2 |
| 2186 | B | 376.5 | 377.2 |
| 2187 | E | 376.5 | 377.2 |
| 2188 | F | 376.5 | 377.2 |
| 2189 | F | 376.5 | 377.2 |
| 2190 | D | 390.5 | 391.5 |
| 2191 | B | 394.5 | 395.5 |
| 2192 | B | 410.9 | 411.2 |
| 2193 | E | 361.5 | 362.5 |
| 2194 | D | 412.5 | 413.2 |
| 2195 | C | 402.5 | 403.2 |
| 2196 | D | 426.5 | 427.5 |
| 2197 | A | 394.5 | 395.4 |
| 2198 | B | 406.5 | 407.5 |
| 2199 | A | 406.5 | 407.5 |
| 2200 | C | 422.5 | 423.5 |
| 2201 | A | 408.5 | 409.5 |
| 2202 | B | 408.5 | 409.5 |
| 2203 | D | 425.0 | 425.4 |
| 2204 | C | 375.5 | 376.6 |
| 2205 | D | 436.5 | 437.2 |
| 2206 | E | 406.5 | 407.1 |
| 2207 | D | 391.4 | 392.1 |
| 2208 | D | 405.5 | 406.1 |
| 2209 | D | 408.5 | 409.1 |
| 2210 | C | 426.5 | 427.4 |
| 2211 | F | 418.5 | 419.1 |
| 2212 | B | 433.6 | 434.2 |
| 2213 | B | 433.6 | 434.2 |
| 2214 | C | 468.0 | 468.1 |
| 2215 | F | 414.9 | 415.1 |
| 2216 | B | 422.5 | 423.3 |
| 2252 | C | 438.6 | 439.4 |
| 2253 | E | 412.5 | 414.2 |
| 2254 | B | 436.5 | 437.2 |
| 2255 | H1 | 361.5 | 362.5 |
| 2256 | A | 457.4 | 458.2 |
| 2257 | B | 453.5 | 454.3 |
| 3253 | D | 350.5 | 351.4 |
| 3254 | C | 364.5 | 365.2 |
| 3255 | C | 364.5 | 365.2 |
| 4259 | C | 4363.5 | 4364.1 |
| 4260 | B | 377.5 | 378.4 |
| 4261 | A | 377.5 | 378.5 |
| 4262 | E | 362.5 | 363.2 |
| 4263 | F | 378.5 | 379.2 |
| 4264 | F | 385.9 | 386.2 |
| 4265 | B | 445.5 | 446.4 |
| 4266 | D | 468.5 | 469.4 |
| 4267 | A | 400.5 | 401.5 |
| 4268 | A | 411.9 | 412.1 |
| 4269 | B | 375.5 | 376.4 |
| 4270 | A | 403.5 | 404.2 |
| 4271 | D | 378.5 | 379.2 |
| 4272 | E | 378.5 | 379.5 |
| 4273 | E | 378.5 | 379.2 |
| 4274 | D | 417.5 | 418.2 |
| 4275 | D | 417.5 | 418.2 |
| 4276 | C | 389.5 | 390.2 |
| 4277 | B | 389.5 | 390.2 |
| 4278 | B | 403.5 | 404.1 |
| 4279 | E | 379.4 | 380.1 |
| 4280 | F | 379.4 | 380.1 |
| 4281 | C | 445.5 | 446.1 |
| 4282 | D | 374.4 | 375.2 |
| 4283 | D | 374.4 | 375.2 |
| 4284 | B | 376.5 | 377.5 |
| 4285 | A | 410.9 | 411.1 |
| 4286 | C | 376.5 | 377.2 |
| 4287 | F | 332.4 | 333.4 |
| 4288 | E | 375.5 | 376.5 |
| 4289 | C | 390.5 | 391.2 |
| 4293 | C | 378.5 | 379.2 |
| 4294 | F | 402.5 | 403.2 |
| 4295 | F | 402.5 | 403.2 |
| 4296 | D | 440.0 | 440.1 |
| 4297 | B | 431.6 | 432.2 |
| 4298 | A | 400.5 | 401.2 |
| 4299 | B | 405.5 | 406.3 |
| 4300 | B | 434.9 | 435.1 |
| 4301 | C | 387.4 | 388.2 |
| 4302 | D | 390.5 | 391.2 |
| 4303 | B | 433.9 | 434.2 |
| 4306 | D | 386.5 | 387.2 |
| 4307 | C | 418.5 | 419.2 |
| 4308 | E | 418.5 | 419.2 |
| 4309 | B | 449.0 | 449.2 |
| 4319 | B | 414.5 | 415.2 |
| 4320 | B | 414.5 | 415.2 |
| 4408 | B | 400.5 | 401.2 |
| 4409 | C | 375.5 | 376.4 |
| 4410 | C | 374.5 | 375.4 |
| 4411 | C | 401.5 | 402.5 |
| 4412 | B | 400.5 | 401.5 |
| 4413 | D | 415.5 | 416.3 |
| 4414 | B | 391.5 | 392.6 |
| 4415 | C | 395.5 | 396.3 |
| 4416 | C | 391.5 | 392.5 |
| 4417 | C | 391.5 | 392.5 |
| 4418 | C | 375.5 | 376.5 |
| 4419 | E | 391.5 | 392.2 |
| 4420 | D | 388.5 | 389.2 |
| 4421 | C | 388.5 | 389.1 |
| 4422 | E | 360.4 | 361.2 |
| 4423 | E | 360.4 | 361.3 |
| 4424 | B | 391.5 | 392.5 |
| 4425 | C | 391.5 | 392.5 |
| 4426 | F | 348.4 | 349.2 |
| 4427 | F | 382.9 | 383.1 |
| 4428 | D | 424.9 | 425.2 |
| 4429 | D | 401.5 | 402.2 |
| 4430 | A | 400.5 | 401.2 |
| 4431 | B | 400.5 | 401.2 |
| 4432 | D | 420.9 | 421.1 |
| 4433 | D | 430.9 | 431.1 |
| 4434 | B | 433.9 | 434.2 |
| 4435 | C | 433.9 | 434.2 |
| 4436 | C | 418.5 | 419.2 |
| 4437 | B | 418.5 | 419.2 |
| 4438 | B | 434.9 | 435.2 |
| 4439 | B | 434.9 | 435.2 |
| 4440 | F | 419.9 | 420.2 |
| 4441 | B | 449.0 | 449.2 |
| 4442 | C | 449.0 | 449.2 |
| 4443 | B | 434.9 | 435.1 |
| 4444 | B | 414.5 | 415.2 |
| 4445 | B | 414.5 | 415.2 |
| 4446 | A | 414.5 | 415.2 |
| 4447 | C | 414.5 | 415.2 |
| 4448 | C | 465.0 | 465.2 |
| 4449 | C | 465.0 | 465.1 |
| 4450 | B | 434.9 | 435.2 |
| 4451 | C | 434.9 | 435.2 |
| 4452 | C | 399.5 | 400.2 |
| 4453 | H1 | 478.9 | 479.1 |
| 4454 | H2 | 385.9 | 386.4 |
| 4455 | H2 | 354.5 | 355.4 |
| 4456 | G | 379.5 | 380.5 |
| 4457 | H2 | 393.5 | 394.5 |
| 4458 | H2 | 356.5 | 357.6 |
| 4459 | F | 351.5 | 352.3 |
| 4460 | G | 376.5 | 377.3 |
| 4461 | E | 395.5 | 396.3 |
| 4462 | E | 392.5 | 393.4 |
| 4463 | G | 419.5 | 420.3 |
| 4464 | D | 376.5 | 377.6 |
| 4465 | D | 365.5 | 366.3 |
| 4466 | E | 380.5 | 381.0 |
| 4467 | B | 451.5 | 452.2 |
| 4468 | D | 401.5 | 402.2 |
| 4469 | E | 395.5 | 396.4 |
| 4470 | F | 407.5 | 408.5 |
| 4471 | F | 440.4 | 441.2 |
| 4472 | D | 440.4 | 441.3 |
| 5434 | D | 409.5 | 410.2 |
| 5435 | D | 374.5 | 375.3 |
| 5436 | F | 371.4 | 372.2 |
| 5437 | F | 405.9 | 406.2 |
| 5441 | E | 489.7 | 490.3 |
| 5442 | F | 446.9 | 447.2 |
| 5443 | H1 | 394.5 | 395.2 |
| 8458 | H1 | 429.4 | 430.2 |
| 8459 | B | 445.5 | 445.9 |
| 9001 | B | 440.4 | 441.4 |
| 9002 | C | 441.4 | 442.2 |
| 9003 | D | 441.4 | 442.2 |
| 9004 | C | 441.4 | 442.2 |
| 9005 | A | 454.5 | 455.4 |
| 9006 | B | 468.5 | 469.4 |
| 9007 | A | 468.5 | 469.2 |
| 9008 | A | 472.4 | 473.2 |
| 10001 | D | 462.5 | 463.4 |
| 10002 | D | 480.5 | 481.2 |
| 10003 | E | 480.5 | 481.2 |
| 10004 | C | 480.5 | 481.2 |

Determination of Related Protease Inhibition

In vitro inhibition of related proteases was determined using an (C50 assay based on standard literature methods (see e.g. Shori et al., Biochem. Pharmacol., 1992, 43, 1209; Bouckaert et al., European Journal of Medicinal Chemistry 110 (2016) 181). Human serine protease enzymes Plasma Kallikrein, KLK1, FXa, Plasmin, Thrombin and Trypsin were assayed for enzymatic activity using an appropriate fluorogenic substrate at Km concentration, FXIa at fixed substrate concentration of 100 μM, and various concentrations of the test compound. Protease activity was measured by monitoring the accumulation of liberated fluorescence from the substrate over 5 mi at 25′C. The linear rate of fluorescence increase per minute was expressed as percentage (%) activity. IC50 values were calculated as the concentration of inhibitor giving 50% inhibition of the uninhibited enzyme activity (100%).

Data acquired from this assay are shown in Table 14 using the scale shown in Table 15.

TABLE 14
Enzyme selectivity data

| | IC₅₀ | IC₅₀ | IC₅₀ | IC₅₀ | IC₅₀ | IC₅₀ | IC₅₀ |
|---|---|---|---|---|---|---|---|
| | (human | (human | (human | (human | (human | (human | (human |
| Example | PKal) | KLK1) | FXa) | FXla) | thrombin) | plasmin) | trypsin) |
| number | nM | nM | nM | nM | nM | nM | nM |
| 1001 | D | | D | | D | G3 | F |
| 1002 | | | F | | G3 | G3 | G3 |
| 1003 | F | | | | | | |
| 1004 | G3 | | | | | | |
| 1005 | E | | E | G3 | F | | |
| 1006 | D | | D | G3 | E | | |
| 1008 | F | | G2 | G2 | G2 | | |
| 1009 | E | | G3 | G3 | G3 | | |
| 1010 | E | | F | G3 | F | | |
| 1011 | F | | | | | | |
| 1012 | D | | F | G2 | E | | |
| 1013 | E | | E | G2 | E | | |
| 1029 | F | E | F | | F | | |
| 1030 | F | E | F | | F | | |
| 1033 | D | C | E | | E | | |
| 1034 | G3 | F | G3 | | G3 | | |
| 1035 | G3 | F | G3 | | G3 | | |
| 1036 | F | F | G3 | | G3 | | |
| 1037 | F | E | G3 | G3 | F | | |
| 1039 | G3 | | | | | | |
| 1040 | G3 | | | | | | |
| 1042 | G1 | | | | | | |
| 1043 | E | | | | | | |
| 1044 | E | | | | | | |
| 1049 | G3 | | | | | | |
| 1050 | G3 | | | | | | |
| 1052 | G3 | | | | | | |
| 1096 | G3 | F | F | | F | | |
| 1102 | F | | | | | | |
| 1103 | F | | | | | | |
| 1116 | G4 | | | | | | |
| 1118 | G3 | | | | | | |
| 1119 | F | | | | | | |
| 1128 | G3 | E | G3 | G3 | F | G3 | G3 |
| 1129 | G3 | | | | | | |
| 1130 | G3 | | | | | | |
| 1131 | F | | | | | | |
| 1133 | G3 | | | | | | |
| 1135 | G3 | | | | | | |
| 1137 | G3 | | | | | | |
| 1140 | G3 | | | | | | |
| 1150 | G3 | | | | | | |
| 1156 | G3 | | | | | | |
| 1157 | G3 | | | | | | |
| 1163 | G3 | | | | | | |
| 1167 | G3 | | | | | | |
| 1176 | G3 | | | | | | |
| 1177 | G4 | | | | | | |
| 1178 | G4 | | | | | | |
| 1180 | G4 | | | | | | |
| 1181 | G3 | | | | | | |
| 1182 | G3 | | | | | | |
| 1183 | G4 | | | | | | |
| 1184 | G4 | | | | | | |
| 1185 | G3 | | | | | | |
| 1186 | G4 | | | | | | |
| 1187 | G4 | | | | | | |
| 1188 | G4 | | | | | | |
| 1189 | G3 | | | | | | |
| 1190 | G4 | | | | | | |
| 1191 | G4 | | | | | | |
| 1192 | G4 | | | | | | |
| 1193 | E | | | | | | |
| 1194 | G3 | | | | | | |
| 1195 | E | | | | | | |
| 1196 | E | | | | | | |
| 1197 | G3 | G3 | G3 | G3 | G3 | G3 | G3 |
| 1198 | G3 | | | | | | |
| 1199 | G4 | | | | | | |
| 1200 | G4 | | | | | | |
| 1201 | G3 | | | | | | |
| 1202 | G3 | G3 | F | | F | | |
| 1203 | G4 | | | | | | |
| 1204 | G4 | | | | | | |
| 1205 | G4 | | | | | | |
| 1206 | G4 | | | | | | |
| 1207 | G4 | | | | | | |
| 1208 | G3 | | | | | | |
| 1209 | G3 | G3 | G3 | | G3 | | |
| 1210 | G4 | | | | | | |
| 1211 | F | | | | | | |
| 1212 | G4 | | | | | | |
| 1213 | F | G4 | G3 | | F | | |
| 1214 | G4 | | | | | | |
| 1215 | G4 | | | | | | |
| 1216 | G4 | | | | | | |
| 1217 | G3 | | | | | | |
| 1218 | G4 | | | | | | |
| 1219 | G4 | | | | | | |
| 1220 | G3 | | | | | | |
| 1221 | G4 | | | | | | |
| 1222 | G3 | | | | | | |
| 1223 | G3 | | | | | | |
| 1224 | G3 | G3 | G3 | | G4 | | |
| 1225 | G4 | | | | | | |
| 1226 | G4 | | | | | | |
| 1227 | G4 | | | | | | |
| 1232 | F | | | | | | |
| 1243 | G3 | F | F | | G3 | | |
| 1244 | G4 | | | | | | |
| 1245 | E | | | | | | |
| 1246 | G3 | | | | | | |
| 1247 | G4 | | | | | | |
| 1248 | G2 | | | | | | |
| 1249 | G3 | | | | | | |
| 1250 | G3 | E | G3 | | G3 | | |
| 1251 | F | F | D | | E | | |
| 1252 | G3 | | | | | | |
| 1253 | G3 | | | | | | |
| 1254 | G3 | | | | | | |
| 1255 | G3 | | | | | | |
| 1256 | G3 | | | | | | |
| 1257 | G3 | | | | | | |
| 1258 | G3 | | | | | | |
| 1259 | G3 | | | | | | |
| 1260 | G3 | | | | | | |
| 1261 | G3 | | | | | | |
| 1262 | E | | | | | | |
| 1263 | G3 | | | | | | |
| 1264 | G3 | | | | | | |
| 1265 | G3 | | | | | | |
| 1266 | G3 | | | | | | |
| 1267 | G3 | | | | | | |
| 1268 | G3 | | | | | | |
| 1269 | G3 | | | | | | |
| 1270 | G3 | | | | | | |
| 1271 | F | | | | | | |
| 1272 | F | | | | | | |
| 1273 | G3 | | | | | | |
| 1274 | G3 | D | G3 | | F | | |
| 1275 | G3 | E | G3 | | G3 | | |
| 1276 | G3 | | | | | | |
| 1277 | G3 | F | G3 | | F | | |
| 1278 | G3 | | | | | | |
| 1279 | G3 | E | G3 | | G3 | | |
| 1280 | G3 | | | | | | |
| 1281 | G3 | | | | | | |
| 1282 | G3 | E | G1 | | | | |
| 1283 | G3 | | | | | | |
| 1284 | | E | G3 | | | | |
| 1285 | | E | G3 | | | | |
| 1286 | | D | G3 | | | | |
| 1287 | | D | G3 | | | | |
| 1288 | | G4 | G3 | | | | |
| 1289 | | G3 | G3 | | | | |
| 1290 | | G3 | G3 | | | | |
| 1291 | | F | G3 | | | | |
| 1292 | | G3 | G3 | | | | |
| 1293 | | G3 | G3 | | | | |
| 1294 | G3 | E | G3 | | | | |
| 1295 | F | E | G3 | | | | |
| 1296 | F | E | G3 | | | | |
| 1297 | G3 | D | G3 | | | | |
| 1298 | G3 | F | G3 | | | | |
| 1299 | G3 | E | G3 | | | | |
| 1300 | G3 | E | G3 | | | | |
| 1301 | G3 | E | G3 | | | | |
| 1302 | F | D | G3 | | | | |
| 1303 | | G1 | | | | | |
| 1304 | | E | | | | | |
| 1305 | | G1 | | | | | |
| 1306 | | D | | | | | |
| 1307 | | E | | | | | |
| 1308 | | E | | | | | |
| 1309 | | G1 | | | | | |
| 1310 | | D | | | | | |
| 1311 | | E | | | | | |
| 1312 | G3 | | | | | | |
| 1313 | | G1 | | | | | |
| 2177 | D | | E | G3 | E | | |
| 2178 | F | | G3 | G3 | G3 | | |
| 2181 | F | C | F | | E | | |
| 2182 | G3 | F | G3 | | G3 | | |
| 2183 | G3 | F | G3 | | G3 | | |
| 2184 | G3 | E | G3 | | G3 | | |
| 2185 | E | | E | G3 | E | | |
| 2186 | F | | F | G3 | G3 | | |
| 2187 | F | | | | | | |
| 2188 | F | | | | | | |
| 2189 | F | | | | | | |
| 2190 | E | | | | | | |
| 2191 | F | | F | G3 | G3 | | |
| 2192 | E | | D | G3 | E | | |
| 2193 | G2 | | G2 | G2 | G2 | | |
| 2194 | F | | F | G3 | F | | |
| 2195 | E | | F | G3 | F | | |
| 2196 | F | | F | G3 | F | | |
| 2197 | G2 | | G2 | G2 | G2 | | |
| 2198 | E | | E | G2 | F | | |
| 2199 | E | | E | G2 | F | | |
| 2201 | F | | G2 | G2 | G2 | | |
| 2202 | D | | E | G2 | E | | |
| 2206 | F | F | G3 | | G3 | | |
| 2207 | E | F | F | | F | | |
| 2208 | G3 | F | F | | F | | |
| 2209 | G3 | F | G3 | | G3 | | |
| 2211 | G3 | F | G3 | | G3 | | |
| 2212 | E | F | G3 | G3 | G3 | G3 | G3 |
| 2213 | F | F | G3 | G3 | G3 | | |
| 2214 | F | E | G3 | | G3 | | |
| 2215 | G3 | F | G3 | | G3 | | |
| 2216 | D | | | | | | |
| 2253 | G3 | | | | | | |
| 2254 | G3 | F | F | | G3 | | |
| 3254 | E | | E | G2 | E | | |
| 4259 | F | F | E | G | D | G3 | F |
| 4260 | G2 | | G2 | G2 | G2 | | |
| 4261 | E | | C | G2 | E | | |
| 4262 | F | F | F | G3 | G3 | G3 | G3 |
| 4265 | | C | D | | C | | |
| 4267 | E | C | B | | C | | |
| 4268 | F | D | E | G3 | G3 | G3 | G3 |
| 4269 | E | C | A | | C | | |
| 4270 | E | C | A | | D | | |
| 4271 | F | F | C | G3 | F | G3 | G3 |
| 4272 | G3 | G3 | G3 | | G3 | | |
| 4273 | G3 | F | E | | G3 | | |
| 4274 | G3 | E | D | | F | | |
| 4275 | F | D | C | | C | | |
| 4276 | G3 | E | E | | G3 | | |
| 4277 | E | D | C | | D | | |
| 4278 | G3 | E | D | | G3 | | |
| 4279 | G3 | F | G3 | | G3 | | |
| 4280 | G3 | F | G3 | | G3 | | |
| 4281 | G3 | E | G3 | | G3 | | |
| 4282 | G3 | E | F | | G3 | | |
| 4283 | E | C | E | | D | | |
| 4284 | F | D | C | | F | | |
| 4285 | G3 | E | F | | G3 | | |
| 4286 | F | E | G3 | | G3 | | |
| 4289 | G3 | F | G3 | | G3 | | |
| 4293 | G3 | | | | | | |
| 4294 | G3 | F | G3 | | G3 | | |
| 4295 | G3 | F | G3 | | G3 | | |
| 4297 | G1 | | | | | | |
| 4298 | G3 | E | | F | G3 | | |
| 4299 | G1 | | | | | | |
| 4300 | F | | | | | | |
| 4301 | G1 | | | | | | |
| 4302 | F | | | | | | |
| 4303 | G3 | | | | | | |
| 4306 | F | | | | | | |
| 4307 | G1 | | | | | | |
| 4308 | G3 | F | G3 | G3 | G3 | G3 | G3 |
| 4309 | G1 | | | | | | |
| 4319 | E | E | F | | G3 | | |
| 4320 | E | | | | | | |
| 4426 | G3 | | | | | | |
| 4427 | G3 | | | | | | |
| 4428 | G1 | | | | | | |
| 4429 | E | | | | | | |
| 4430 | G1 | | | | | | |
| 4431 | G1 | | | | | | |
| 4432 | G3 | | | | | | |
| 4433 | G3 | | | | | | |
| 4434 | G3 | E | E | | G3 | | |
| 4435 | G3 | | | | | | |
| 4436 | G3 | | | | | | |
| 4437 | G3 | E | E | G3 | F | G3 | G3 |
| 4438 | F | E | F | G3 | F | G3 | G3 |
| 4439 | F | | | | | | |
| 4440 | G3 | | | | | | |
| 4441 | F | D | G3 | | G3 | | |
| 4442 | F | | | | | | |
| 4443 | G3 | | | | | | |
| 4444 | E | | | | | | |
| 4445 | E | | | | | | |
| 4446 | E | E | E | F | G3 | G3 | F |
| 4447 | E | | | | | | |
| 4448 | G3 | | | | | | |
| 4449 | G3 | | | | | | |
| 4450 | F | | | | | | |
| 4451 | G3 | | | | | | |
| 4452 | G3 | | | | | | |
| 4453 | G3 | | | | | | |
| 4454 | G4 | D | G3 | G3 | G3 | G3 | G3 |
| 4455 | G4 | | | | | | |
| 4456 | F | | | | | | |
| 4457 | G4 | | | | | | |
| 4458 | G4 | | | | | | |
| 4459 | G4 | | | | | | |
| 4460 | G4 | | | | | | |
| 4461 | G3 | | | | | | |
| 4462 | G4 | | | | | | |
| 4463 | G4 | | | | | | |
| 4464 | G2 | E | G4 | | G1 | | |
| 4465 | G4 | | | | | | |
| 4466 | G4 | | | | | | |
| 4467 | G3 | E | F | | F | | |
| 4468 | G3 | | | | | | |
| 4469 | G3 | | | | | | |
| 4470 | G3 | | | | | | |
| 5434 | D | | D | G3 | D | | |
| 5435 | F | D | E | | E | | |
| 5436 | G3 | | | | | | |
| 5437 | G3 | | | | | | |
| 5441 | G3 | | | | | | |
| 5442 | G3 | | | | | | |
| 9001 | G3 | E | G3 | | | | |
| 9002 | | E | | | | | |
| 9003 | | E | | | | | |
| 9004 | | E | | | | | |
| 9005 | | G1 | | | | | |
| 9006 | G3 | G3 | G3 | | | | |
| 10001 | G3 | | | | | | |
| 10002 | | F | G3 | | | | |
| 10003 | | G3 | G3 | | | | |
| 10004 | | F | G3 | | | | |

TABLE 15
Scale used to present enzyme selectivity data

| Category | IC₅₀ (nM) |
|---|---|
| A | 10-100 |
| B | 100-300 |
| C | 300-1,000 |
| D | 1,000-3,000 |
| E | 3,000-10,000 |
| F | 10,000-40,000 |
| G1 | >4,000 |
| G2 | >20,000 |
| G3 | >40,000 |
| G4 | >100,000 |

Pharmacokinetics

Pharmacokinetic studies of the compounds in Table 16 were performed to assess the pharmacokinetics following a single intravenous dose and a single oral dose in male Sprague-Dawley rats. Two rats were given a single intravenous dose of 1 mL/kg of a nominal 1 mg/mL (1 mg/kg) composition of test compound in 10% DMSO/10% Cremophor EL/80% SWFI vehicle. Example 2191 was dosed at 1 mL/kg of a nominal 2 mg/mL (2 mg/kg) using the same vehicle.

Two rats were given a single oral dose of 5 mL/kg of a nominal 1 mg/mL (5 mg/kg) composition of test compound in 10% DMSO/10% Cremophor EL/80% SWFI vehicle. Example 2191 was dosed at 5 mL/kg of a nominal 2 mg/mL (10 mg/kg) using the same vehicle.

Following intravenous dosing, blood samples were collected over a period of 12 h. Sample times were 2, 5, 15 and 30 min then 1, 2, 4, 6, and 12 h. Following oral dosing, blood samples were collected over a period of 24 h. Sample times were 5, 15 and 30 min then 1, 2, 4, 6, 8, 12 and 24 h.

Following collection, blood samples were centrifuged and the plasma fraction analysed for concentration of test compound by LCMS. Oral bioavailability and half-life calculations from these studies were obtained using Phoenix WinNonlin (v8.0) and are shown below:

TABLE 16
Oral exposure data

| Example | Dose iv | Dose po | | |
|---|---|---|---|---|
| Number | (mg/kg) | (mg/kg) | F % | T_{1/2} (min) |
| 1029 | 1.1 | 5.3 | 9.7 | 370.7 |
| 1243 | 1.5 | 7.7 | 4.9 | 525.9 |
| 1274 | 1.0 | 4.9 | 32.1 | 151.4 |
| 1277 | 0.9 | 4.3 | 25.0 | 81.0 |
| 1282 | 1.1 | 5.6 | 53.2 | 140.7 |
| 1305 | 0.8 | 4.1 | 79.4 | 242.5 |
| 2186 | 1.1 | 5.6 | 15.8 | 263.4 |
| 2191 | 2.6 | 12.4 | 22.8 | 541.1 |
| 2197 | 1.0 | 5.0 | 26.6 | 412.4 |
| 2212 | 1.6 | 7.9 | 6.8 | 260.4 |
| 4260 | 0.78 | 4.0 | 13.6 | 598.8 |
| 4268 | 1.1 | 5.7 | 3.0 | 745.3 |
| 4299 | 0.7 | 3.6 | 8.3 | 604.2 |
| 4301 | 1.0 | 5.0 | 8.5 | 131.3 |

1. A compound of formula (I), 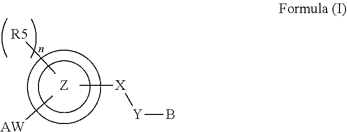
wherein:
Z is a 6- or 5-membered heteroaromatic ring containing 1, 2 or 3 ring members that are, independently, N, S or O; or phenyl; or,
Z is 2-pyridone or 4-pyridone,
X is SO₂ or CR1R2;
R1 is H, alkyl, alkoxy, OH, halo or NR13R14; and
R2 is H or small alkyl; or
R1 and R2, together with the carbon atom to which they are attached, are linked by alkylene to form a 3-, 4-, or 5-membered saturated ring;
Y is NR12, O, o CR3R4;
R3 and R4 are independently H or alkyl; or
X is CR1R2 and Y is CR3R4, and R1 and R3, together with the carbon atom to which R1 is attached and the carbon atom to which R3 is attached, are linked by alkylene to form a 3-, 4-, or 5-membered saturated ring; or
X is CR1R2 and Y is NR12, and R1 and R12, together with the carbon atom to which R1 is attached and the nitrogen atom to which R12 is attached, are linked by alkylene to form a 3-, 4-, or 5-membered saturated heterocycle;
B is:
(i) heteroaryl^{a};
(ii) aryl;
(iii) a 5- to 6-membered non-aromatic heterocyclic ring containing one N ring member, which, where possible, is saturated or unsaturated with 1 or 2 double bonds, wherein the non-aromatic heterocyclic ring is optionally substituted by 1, 2 or 3 substituents that are independently alkyl, alkoxy, aryl^{b}, OH, OCF₃, halo, oxo, CN, or CF₃; or
(iv) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring is optionally substituted with 1, 2, or 3 substituted by up to three substituents that are independently alkyl, alkoxy, OH, OCF₃, halo, oxo, CN, or CF₃, wherein the 6,5-bicyclic ring is attached via the 6- or 5-membered ring;
n is 0, 1 or 2;
when present, each R5 is independently alkyl, cyclopropyl, alkoxy, halo, OH, CN, (CH₂)₀₋₆COOH, or CF₃;
AW- is: —(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—NR12-(CH₂)₁₋₆—C(═O)-A, —(CH₂)₀₋₆—NH—C(═O)—(CH₂)₀₋₆-A, —C(═O)NR12-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A or —SO₂—NH-A;
A is a 4- to 15-membered mono-, bi-, or tri-cyclic ring system, containing one N ring member and optionally one, two or three further ring members that are independently N, O or S, optionally wherein the ring system is substituted, where possible, with 1, 2, 3 or 4 substituents that are independently halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, or CN;
wherein when A is a bicyclic ring system, the bicyclic ring system is fused, bridged or spiro;
wherein when A is a tricyclic ring system, each of the three rings in the tricyclic ring system is either fused, bridged or spiro to at least one of the other rings in the tricyclic ring system;
alkyl is a linear saturated hydrocarbon having up to 10 carbon atoms (C₁-C₁₀) or a branched saturated hydrocarbon of between 3 and 10 carbon atoms (C₃-C₁₀); alkyl is optionally substituted with 1, 2 or 3 substituents that are independently (C₁-C₆)alkoxy, OH, —NR13R14, —C(═O)OR13, —C(═O)NR13R14, CN, CF₃, or halo;
alkyl^{b} is a linear saturated hydrocarbon having up to 10 carbon atoms (C₁-C₁₀) or a branched saturated hydrocarbon of between 3 and 10 carbon atoms (C₃-C₁₀);
alkyl^{b} is optionally substituted with 1, 2 or 3 substituents that are independently (C₁-C₆)alkoxy, OH, CN, CF₃, or halo;
small alkyl is a linear saturated hydrocarbon having up to 4 carbon atoms (C₁-C₄) or a branched saturated hydrocarbon of between 3 and 4 carbon atoms (C₃-C₄); small alkyl is optionally substituted with 1 or 2 substituents that are independently (C₁-C₆)alkoxy, OH, NR13R14, C(═O)OR13, C(═O)NR13R14, CN, CF₃, or halo;
small alkyl^{b} is linear saturated hydrocarbon having up to 4 carbon atoms (C₁-C₄) or a branched saturated hydrocarbon of between 3 and 4 carbon atoms (C₃-C₄); small alkyl^{b} is optionally substituted with 1 or 2 substituents that are independently (C₁-C₆)alkoxy, OH, CN, CF₃, or halo;
alkylene is a bivalent linear saturated hydrocarbon having 1 to 5 carbon atoms (C₁-C₅); alkylene is optionally substituted with 1 or 2 substituents that are independently alkyl^{b}, (C₁-C₆)alkoxy, OH, CN, CF₃, or halo;
aryl is phenyl, biphenyl or naphthyl; aryl is optionally substituted with 1, 2 or 3 substituents that are independently alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ or CF₃;
aryl^{b} is phenyl, biphenyl or naphthyl; aryl^{b} is optionally substituted with 1, 2 or 3 substituents that are independently alkyl^{b}, alkoxy, OH, halo, CN, or CF₃;
cycloalkyl is a monocyclic saturated hydrocarbon ring of between 3 and 6 carbon atoms (C₃-C₆); cycloalkyl is optionally substituted with 1 or 2 substituents that are independently alkyl, (C₁-C₆)alkoxy, OH, CN, CF₃, or halo;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy is optionally substituted with 1 or 2 substituents that are independently OH, CN, CF₃, or fluoro;
halo is F, Cl, Br, or I;
heteroaryl is a 5- or 6-membered carbon-containing aromatic ring containing one, two or three ring members that are N, NR8, S, or O;
heteroaryl is optionally substituted with 1, 2 or 3 substituents that are independently alkyl, alkoxy, OH, OCF₃, halo, CN, 9 g CF₃;
heteroaryl^{a} is a 5, 6, 9 or 10 membered mono- or bi-cyclic aromatic ring, containing, where possible, 1, 2, 3 or 4 ring members that are independently N, NR12, S or O; heteroaryl^{a} is optionally substituted with 1, 2 or 3 substituents that are independently alkyl, alkoxy, OH, OCF₃, halo, CN, aryl^{b}, —(CH₂)₀₋₃—NR13R14, heteroaryl^{b}, —C(═O)OR12, —C(═O)NR13R14 or CF₃;
heteroaryl^{b} is a 5, 6, 9 or 10 membered mono- or bi-cyclic aromatic ring, containing, where possible, 1, 2 or 3 ring members that are independently or N, NR12, S or O; wherein heteroaryl^{b} is optionally substituted with 1, 2 or 3 substituents that are independently alkyl^{b}, alkoxy, OH, halo, CN, aryl^{b}, —(CH₂)₁₋₃-aryl^{b}, or CF₃;
heterocycloalkyl is a non-aromatic carbon-containing monocyclic ring containing 5, 6, or 7 ring members, wherein one or two ring members are independently N, NR8, S, SO, SO₂, or O; wherein heterocycloalkyl is optionally substituted with 1, 2, or 3 substituents that are independently alkyl, alkoxy, OH, OCF₃, halo, oxo or CN;
R8 is independently H, alkyl, cycloalkyl, or heterocycloalkyl^{a};
heterocycloalkyl^{a} is a non-aromatic carbon-containing monocyclic ring containing 3, 4, 5, or 6, ring members, wherein at least one ring member is independently N, NR12, S, or O; heterocycloalkyl^{a} is optionally be substituted with 1 or 2 substituents that are independently alkyl, (C₁-C₆)alkoxy, OH, CN, CF₃, or halo;
R12 is independently H, alkyl, or cycloalkyl;
R13 and R14 are independently H, alkyl^{b}, aryl^{b} or heteroaryl^{b} or R13 and R14 together with the nitrogen atom to which they are attached form a carbon-containing 4-, 5-, 6- or 7-membered heterocyclic ring, optionally containing an additional heteroatom that is N, NR12, S, SO, SO₂, or O, which is saturated or unsaturated with 1 or 2 double bonds and which is optionally mono- or di-substituted with substituents that are oxo, alkyl^{b}, alkoxy, OH, halo or CF₃;
or a tautomer, isomer, stereoisomer, deuterated isotope, or pharmaceutically acceptable salt and/or solvate thereof.

2. The compound of formula (I) according to claim 1 or a tautomer, isomer, stereoisomer, a deuterated isotope, and a pharmaceutically acceptable salt and/or solvate thereof,
wherein Z is a 6- or 5-membered heteroaromatic ring containing 1 or 2 ring members that are independently N or S; or phenyl.

3. The compound of formula (I) according to claim 2 or a tautomer, isomer, stereoisomer, a deuterated isotope, and a pharmaceutically acceptable salt and/or solvate thereof,
wherein Z is pyrazole, phenyl, pyrimidine, pyridine, pyrazine, pyridazine, oxazole, thiophene, or thiazole.

4. The compound of formula (I) according to claim 1 or a tautomer, isomer, stereoisomer, a deuterated isotope, and a pharmaceutically acceptable salt and/or solvate thereof,
wherein the compound is:

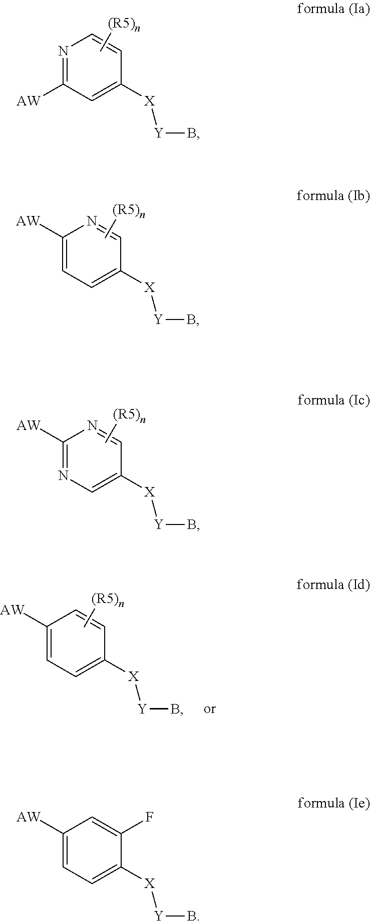

5. The compound of formula (I) according to claim 1, or a tautomer, isomer, stereoisomer, a deuterated isotope, and a pharmaceutically acceptable salt and/or solvate thereof,
wherein X is CR1R2.

6. The compound of formula (I) according to claim 1, or a tautomer, isomer, stereoisomer, a deuterated isotope, and a pharmaceutically acceptable salt and/or solvate thereof,
wherein Y is NR12.

7. The compound of formula (I) according to claim 6 or a tautomer, isomer, stereoisomer, a deuterated isotope, and a pharmaceutically acceptable salt and/or solvate thereof,
wherein Y is NH.

8. The compound of formula (I) according to claim 1 or a tautomer, isomer, stereoisomer, a deuterated isotope, and a pharmaceutically acceptable salt and/or solvate thereof,
wherein B is heteroaryl^{a}.

9. The compound of formula (I) according to claim 8 or a tautomer, isomer, stereoisomer, a deuterated isotope, and a pharmaceutically acceptable salt and/or solvate thereof,
wherein Y is attached to B at a carbon atom on the heteroaryl^{a} ring, and the two ring atoms adjacent to the carbon atom on the heteroaryl^{a} ring to which Y attaches are both carbon.

10. The compound of formula (I) according to claim 8 or a tautomer, isomer, stereoisomer, a deuterated isotope, and a pharmaceutically acceptable salt and/or solvate thereof,
wherein B is:
isoquinolinyl, substituted with NH₂ at the 1-position 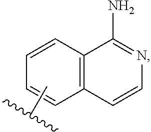
optionally further substituted with 1 or 2 substituents as for heteroaryl^{a};
6-azaindolyl 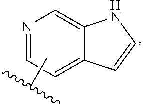
optionally substituted as for heteroaryl^{a};
7-azaindolyl 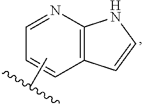
optionally substituted as for heteroaryl^{a}; or
pyridyl 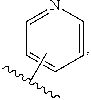
optionally substituted as for heteroaryl^{a}.

11. The compound of formula (I) according to claim 8 or a tautomer, isomer, stereoisomer, a deuterated isotope, and a pharmaceutically acceptable salt and/or solvate thereof,
wherein B is:
isoquinolinyl, substituted with NH₂ at the 1-position, that is 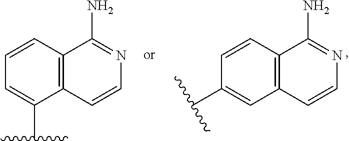 [SMILES: *c1cccc2c(N)nccc12 ; *c1ccc2c(N)nccc2c1]
optionally further substituted with 1 or 2 substituents as for heteroaryl^{a};
6-azaindolyl 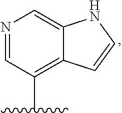 [SMILES: *c1cncc2[nH]ccc12]
optionally substituted as for heteroaryl^{a};
7-azaindolyl 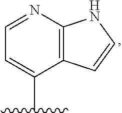 [SMILES: *c1ccnc2[nH]ccc12]
optionally substituted as for heteroaryl^{a}; or
pyridyl 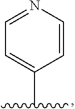 [SMILES: *c1ccncc1]
optionally substituted as for heteroaryl^{a}.

12. The compound of formula (I) according to claim 1 or a tautomer, isomer, stereoisomer, a deuterated isotope, and a pharmaceutically acceptable salt and/or solvate thereof,
wherein n is 0 or 1.

13. The compound of formula (I) according to claim 1 or a tautomer, isomer, stereoisomer, a deuterated isotope, and a pharmaceutically acceptable salt and/or solvate thereof,
wherein AW is -A, —OCH₂-A, —CH₂O-A, —O-A, —(CH₂)₂-A, —NH—CH₂-A or —NH—(CH₂)₂—C(═O)-A.

14. The compound of formula (I) according to claim 1 or a tautomer, isomer, stereoisomer, a deuterated isotope, and a pharmaceutically acceptable salt and/or solvate thereof,
wherein A is a 4- to 12-membered mono- or bi-cyclic ring system, containing one N ring member and optionally one, two or three further ring members that are independently N, O or S, optionally wherein the ring system is substituted, where possible, with 1, 2, 3 or 4 substituents that are independently halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, or CN;
wherein when A is a bicyclic ring system, the bicyclic ring system is fused, bridged or spiro.

15. The compound of formula (I) according to claim 14 or a tautomer, isomer, stereoisomer, a deuterated isotope, and a pharmaceutically acceptable salt and/or solvate thereof,
wherein A is a 4- to 12-membered mono- or bi-cyclic ring system, containing one N ring member and optionally one or two further ring members that are independently N or O, optionally wherein the ring system is substituted, where possible, with 1, 2, 3 or 4 substituents that are independently halo, alkyl, OH, oxo, cycloalkyl, alkoxy, —(CH₂)₀₋₂-heteroaryl, heterocycloalkyl^{a}, C(═O)R12, C(═O)OR13, C(═O)NR13R14, NR13R14, CF₃, or CN;
wherein when A is a bicyclic ring system, the bicyclic ring system is fused, bridged or spiro.

16. The compound of formula (I) according to claim 15 or a tautomer, isomer, stereoisomer, a deuterated isotope, and a pharmaceutically acceptable salt and/or solvate thereof,
wherein A is:

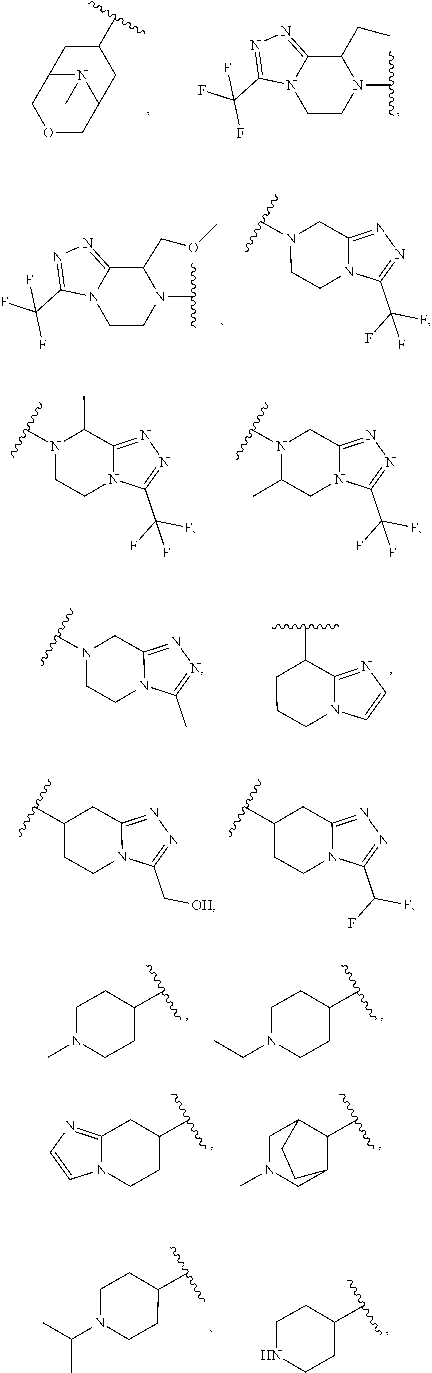

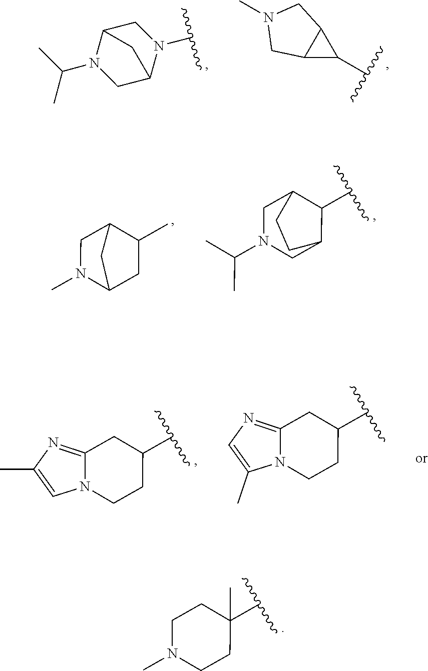

17. A compound selected from Table 1a, 1b, 2a, 2b, 3, 4a, 4b, 5a, 5b, 6, 7, 8a, 8b, 8c, 9, and 10, or a pharmaceutically acceptable salt, solvate, or solvate of a salt thereof.

18. A pharmaceutical composition comprising: a compound, or a pharmaceutically acceptable salt and/or solvate thereof, according to claim 1, and at least one pharmaceutically acceptable excipient.

19. (canceled)

20. A method for treating a disease or condition in which Factor XIIa activity is implicated, comprising administering the compound, or a pharmaceutically acceptable salt and/or solvate thereof, of claim 1 to a patient in need thereof.

21. The method of claim 20, wherein the disease or condition in which Factor XIIa activity is implicated is a bradykinin-mediated angioedema, wherein the bradykinin-mediated angioedema is hereditary angioedema.

22. The method of claim 20, wherein the disease or condition in which Factor XIIa activity is implicated is a bradykinin-mediated angioedema, wherein the bradykinin-mediated angioedema is non hereditary.

23. The method of claim 20, wherein, the disease or condition in which Factor XIIa activity is implicated is a thrombotic disorder.

24. A compound of formula (II), 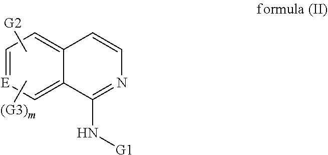
wherein:
E is CH or N;
G1 is either: 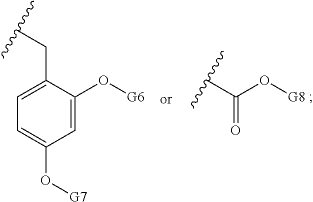
G2 is F, Cl, or Br;
m is 0, 1 or 2;
G3, when present, is independently alkyl, OH, OCF₃, aryl, heteroaryl^{b}, alkoxy, CF₃, CN, —(CH₂)₀₋₃—N(G4)(G5), —C(═O)OR12, —C(═O)NR13R14 or halo; provided that when m is 1, G3 is not methyl;
G4 and G5 are independently alkyl^{b}, aryl^{b} or heteroaryl^{b} or G4 and G5 together with the nitrogen atom to which they are attached form a carbon-containing 4-, 5-, 6- or 7-membered heterocyclic ring, optionally containing an additional heteroatom that is N, NR12, S, SO, SO₂, or O, which is saturated or unsaturated with 1 or 2 double bonds and which is optionally mono- or di-substituted with substituents that are oxo, alkyl^{b}, alkoxy, OH, halo or CF₃;
G6 and G7 are independently methyl, ethyl, n-propyl o i-propyl;
G8 is methyl, ethyl, n-propyl, i-propyl, n-butyl or i-butyl;
alkyl is a linear saturated hydrocarbon having up to 10 carbon atoms (C₁-C₁₀) or a branched saturated hydrocarbon of between 3 and 10 carbon atoms (C₃-C₁₀); alkyl is optionally substituted with 1, 2 or 3 substituents that are independently (C₁-C₆)alkoxy, OH, —NR13R14, —C(═O)OR13, —C(═O)NR13R14, CN, CF₃, or halo;
alkyl^{b} is a linear saturated hydrocarbon having up to 10 carbon atoms (C₁-C₁₀) or a branched saturated hydrocarbon of between 3 and 10 carbon atoms (C₃-C₁₀);
alkyl^{b} is optionally substituted with 1, 2 or 3 substituents that are independently (C₁-C₆)alkoxy, OH, CN, CF₃, or halo;
aryl^{b} is phenyl, biphenyl or naphthyl; aryl^{b} is optionally substituted with 1, 2 or 3 substituents that are independently alkyl^{b}, alkoxy, OH, halo, CN, or CF₃;
cycloalkyl is a monocyclic saturated hydrocarbon ring of between 3 and 6 carbon atoms (C₃-C₆); cycloalkyl is optionally substituted with 1 or 2 substituents that are independently alkyl, (C₁-C₆)alkoxy, OH, CN, CF₃, or halo;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy is optionally substituted with 1 or 2 substituents that are independently OH, CN, CF₃, or fluoro;
halo is F, Cl, Br, or I;
heteroaryl is a 5- or 6-membered carbon-containing aromatic ring containing one, two or three ring members that are N, NR8, S, or O; heteroaryl is optionally substituted with 1, 2 or 3 substituents that are independently alkyl, alkoxy, OH, OCF₃, halo, CN, or CF₃;
heteroaryl^{a} is a 5, 6, 9 or 10 membered mono- or bi-cyclic aromatic ring, containing, where possible, 1, 2, 3 or 4 ring members that are independently N, NR12, S or O; heteroaryl^{a} is optionally substituted with 1, 2 or 3 substituents that are independently alkyl, alkoxy, OH, OCF₃, halo, CN, aryl^{b}, —(CH₂)₀₋₃—NR13R14, heteroaryl^{b}, —C(═O)OR12, —C(═O)NR13R14 or CF₃;
heteroaryl^{b} is a 5, 6, 9 or 10 membered mono- or bi-cyclic aromatic ring, containing, where possible, 1, 2 or 3 ring members that are independently N, NR12, S or O; wherein heteroaryl^{b} is optionally substituted with 1, 2 or 3 substituents that are independently alkyl^{b}, alkoxy, OH, halo, CN, aryl^{b}, —(CH₂)₁₋₃-aryl^{b}, or CF₃;
heterocycloalkyl is a non-aromatic carbon-containing monocyclic ring containing 5, 6, or 7 ring members, wherein one or two ring members are independently N, NR8, S, SO, SO₂, or O; wherein heterocycloalkyl is optionally substituted with 1, 2, or 3 substituents that are independently alkyl, alkoxy, OH, OCF₃, halo, oxo or CN;
R8 is independently H, alkyl, cycloalkyl, or heterocycloalkyl^{a};
heterocycloalkyl^{a} is a non-aromatic carbon-containing monocyclic ring containing 3, 4, 5, or 6, ring members, wherein at least one ring member is independently N, NR12, S, or O; heterocycloalkyl^{a} is optionally be substituted with 1 or 2 substituents that are independently alkyl, (C₁-C₆)alkoxy, OH, CN, CF₃, or halo;
R12 is independently H, alkyl, or cycloalkyl;
R13 and R14 are independently H, alkyl^{b}, aryl^{b} or heteroaryl^{b} or R13 and R14 together with the nitrogen atom to which they are attached form a carbon-containing 4-, 5-, 6- or 7-membered heterocyclic ring, optionally containing an additional heteroatom that is N, NR12, S, SO, SO₂, or O, which is saturated or unsaturated with 1 or 2 double bonds and which is optionally mono- or di-substituted with substituents that are oxo, alkyl^{b}, alkoxy, OH, halo or CF₃;
or a tautomer, isomer, stereoisomer, deuterated isotope, or salt and/or solvate thereof.

25. A compound that is selected: 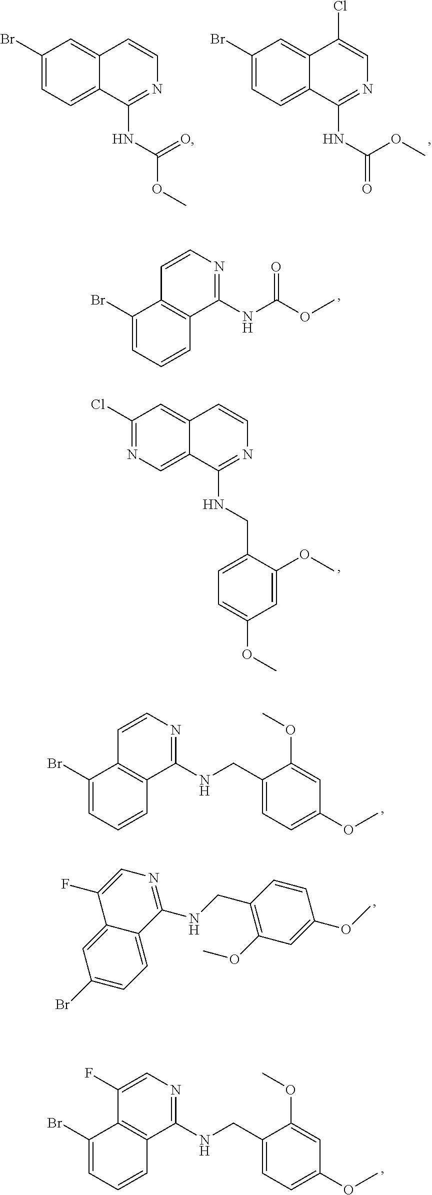
or a salt, solvate, or solvate of a salt thereof.

26. The compound of claim 1, that is a an enantiomer, diastereoisomer, or racemic or scalemic mixture thereof.
